(19) United States
(12) Patent Application Publication
(10) Pub. No.: US 2026/0347805 A1
(43) Pub. Date: Nov. 5, 2026

(54) KOD POLYMERASE VARIANTS WITH URACIL TEMPLATE TOLERANCE

(71) Applicant: Watchmaker Genomics, Inc., Boulder, CO (US)

(72) Inventors: Leonidas Karamanof, Cape Town (ZA); Campbell McDuling, Cape Town (ZA); Bjarne Faurholm, Cape Town (ZA); Eric van der Walt, Cape Town (ZA)

(73) Assignee: Watchmaker Genomics, Inc., Boulder, CO (US)

(21) Appl. No.: 19/555,579

(22) Filed: Mar. 3, 2026

Related U.S. Application Data
(60) Provisional application No. 63/766,640, filed on Mar. 4, 2025.

(51) Int. Cl. C12N 9/12; C12P 19/34

(52) U.S. Cl. CPC C12N 9/1252; C12P 19/34; C12Y 207/07007

(57) ABSTRACT

Provided herein are Thermococcus kodakarensis (KOD) polymerase variants and methods of use thereof. Disclosed KOD polymerase variants include KOD polymerase variants having improved uracil template tolerance, inosine template tolerance, and/or uracil incorporation tolerance.

### RELATED APPLICATIONS

This application claims the benefit under 35 U.S.C. § 119(e) to U.S. Provisional Application No. 63/766,640, filed Mar. 4, 2025, entitled “KOD POLYMERASE VARIANTS WITH URACIL TEMPLATE TOLERANCE”, the contents of which are incorporated herein by reference in their entirety.

### REFERENCE TO AN ELECTRONIC SEQUENCE LISTING

The contents of the electronic sequence listing (W109470031US01-SEQ-ARM.xml; Size: 5,592,198 bytes; and Date of Creation: Mar. 3, 2026) are herein incorporated by reference in their entirety.

### BACKGROUND

DNA polymerases play a central role in modern molecular biology. DNA polymerases synthesize new copies of DNA strands using an already existing DNA strand as a template. Additionally, some DNA polymerases have proofreading/repair domains that can repair errors which occur when new copies of the DNA strand are made. DNA polymerases are important in many applications including the polymerase chain reaction (PCR), DNA sequencing, DNA cloning, synthetic biology, and molecular diagnostics. In a natural system, DNA polymerases operate in cellular physiological conditions and amplify naturally occurring DNA (typically as part of cell replication and DNA repair). However, in molecular biology applications, DNA polymerases may need to operate in conditions that are not physiological and/or amplify a polynucleotide having nucleotides that do not naturally occur in DNA.

Thermococcus kodakarensis (KOD) polymerase is an archaeal B-family thermostable DNA polymerases used in some molecular biology applications, like PCR, which requires drastically changing temperatures (e.g., frequent changes between 60 °C to 98 °C).

### SUMMARY

This disclosure provides KOD polymerase variants that have enhanced uracil (dU) template tolerance (e.g., amplification of a template DNA strand comprising uracil), inosine (dI) template tolerance (e.g., amplification of a template DNA strand comprising inosine), and/or uracil (dUTP) incorporation tolerance (e.g., amplification of a template DNA strand involving incorporation of dUTP into the synthesized strand and/or subsequent amplification the synthesize strand) relative to wildtype KOD polymerase. Wildtype KOD polymerase has little to no dU template tolerance, dI template tolerance, or dUTP incorporation tolerance. Improved KOD polymerase variants are useful in molecular biology applications, such as amplification of polynucleotides via polymerase chain reaction (PCR) for sequencing. Current DNA polymerases lack the ability to efficiently and accurately amplify DNA templates containing uracil or inosine. KOD polymerase variants with enhanced uracil template tolerance, inosine template tolerance, and/or uracil incorporation tolerance can be used to amplify polynucleotides having uracil and/or inosine with greater efficiency compared to wildtype KOD polymerase.

These KOD polymerase variants were identified by screening the amplification efficiency of thousands of different KOD polymerase variants under conditions that incorporated dU and/or dI into template strands via oligonucleotide primers containing dU and/or dI bases or via incorporation of dUTP. The products of extension reactions with these primers or with dUTPs serve as dU and/or dI containing template strands during subsequent cycles of PCR. The screen identified both single amino acid substitution variants and combinatorial amino acid substitution variants of KOD polymerase with improved dU/dI template tolerance and/or dUTP incorporation tolerance.

In some aspects, this disclosure provides a Thermococcus kodakarensis (KOD) polymerase variant comprising one or more of the following substitutions: an aspartate (D), glutamate (E), phenylalanine (F), histidine (H), lysine (K), leucine (L), asparagine (N), threonine (T), valine (V), or tryptophan (W) at a position corresponding to isoleucine 2 (I2) of SEQ ID NO: 1; a D, H, I, serine (S), T, or tyrosine (Y) at a position corresponding to L3 of SEQ ID NO: 1; an I, L, methionine (M), proline (P), or Y at a position corresponding to D4 of SEQ ID NO: 1; a D, E, F, glycine (G), H, L, W, or Y at a position corresponding to T5 of SEQ ID NO: 1; a L at a position corresponding to D6 of SEQ ID NO: 1; a D, E, H, K, N, glutamine (Q), arginine (R), S, or T at a position corresponding to Y7 of SEQ ID NO: 1; a D, E, H, or T at a position corresponding to I8 of SEQ ID NO: 1; an alanine (A), cysteine (C), D, E, F, H, I, L, N, R, or Y at a position corresponding to T9 of SEQ ID NO: 1; a P or Q at a position corresponding to E10 of SEQ ID NO: 1; a Q or T at a position corresponding to D11 of SEQ ID NO: 1; a C, Q, T, or V at a position corresponding to G12 of SEQ ID NO: 1; a G, H, T, V, or W at a position corresponding to K13 of SEQ ID NO: 1; a C, D, E, F, H, M, Q, S, T or W at a position corresponding to P14 of SEQ ID NO: 1; a Q or W at a position corresponding to V15 of SEQ ID NO: 1; a P at a position corresponding to R17 of SEQ ID NO: 1; a Y at a position corresponding to I18 of SEQ ID NO: 1; a W at a position corresponding to E22 of SEQ ID NO: 1; a D at a position corresponding to F34 of SEQ ID NO: 1; a C or T at a position corresponding to P36 of SEQ ID NO: 1; an A, D, E, F, G, S, or T at a position corresponding to Y37 of SEQ ID NO: 1; a L at a position corresponding to V71 of SEQ ID NO: 1; a R at a position corresponding to F87 of SEQ ID NO: 1; a D, P, or W at a position corresponding to H89 of SEQ ID NO: 1; a G at a position corresponding to P90 of SEQ ID NO: 1; a D, E, N, or Y at a position corresponding to 91 of SEQ ID NO: 1; a P at a position corresponding to D92 of SEQ ID NO: 1; a C, D, E, F, G, H, I, M, N, Q, W, or Y at a position corresponding to P94 of SEQ ID NO: 1; a P and R at a position corresponding to I96 of SEQ ID NO: 1; an A, C, D, E, F, G, H, I, K, L, M, N, P, Q, S, T, V, W, or Y at a position corresponding to R97 of SEQ ID NO: 1; an E or Q at a position corresponding to R101 of SEQ ID NO: 1; a D or F at a position corresponding to I109 of SEQ ID NO: 1; a P at a position corresponding to Y110 of SEQ ID NO: 1; a G at a position corresponding to E111 of SEQ ID NO: 1; a D or E at a position corresponding to Y112 of SEQ ID NO: 1; a P at a position corresponding to D113 of SEQ ID NO: 1; an E, G, H, K, N, P, Q, S, or Y at a position corresponding to I114 of SEQ ID NO: 1; a C, D, F, H, N, S, T, or Y at a position corresponding to P115 of SEQ ID NO: 1; an A, D, E, G, H, I, K, N, P, Q, R, or V at a position corresponding to F116 of SEQ ID NO: 1; a D, E, L, M, Q, or W at a position corresponding to A117 of SEQ ID NO: 1; an A, C, D, E, F, G, H, I, K, L, M, N, Q, S, T, V, W, or Y at a position corresponding to R119 of SEQ ID NO: 1; a P at a position corresponding to Y120 of SEQ ID NO: 1; a K, R, or W at a position corresponding to I122 of SEQ ID NO: 1; or an A, C, F, H, I, K, L, M, Q, R, S, T, V, W, or a Y at a position corresponding to D123 of SEQ ID NO: 1.

In some embodiments, the KOD polymerase variant comprises one or more of the following substitutions: a D, E, F, H, K, L, N, T, V, or W at a position corresponding to I2 of SEQ ID NO: 1; a D, H, I, S, T, or Y at a position corresponding to L3 of SEQ ID NO: 1; an I, L, M, P, or Y at a position corresponding to D4 of SEQ ID NO: 1; a D, E, F, G, H, L, W, or Y at a position corresponding to T5 of SEQ ID NO: 1; a L at a position corresponding to D6 of SEQ ID NO: 1; a D, E, H, K, N, Q, R, S, or T at a position corresponding to Y7 of SEQ ID NO: 1; a D, E, H, or T at a position corresponding to I8 of SEQ ID NO: 1; an A, C, D, E, F, H, I, L, N, R, or Y at a position corresponding to T9 of SEQ ID NO: 1; a P or Q at a position corresponding to E10 of SEQ ID NO: 1; a Q or T at a position corresponding to D11 of SEQ ID NO: 1; a C, Q, T, or V at a position corresponding to G12 of SEQ ID NO: 1; a G, H, T, V, or W at a position corresponding to K13 of SEQ ID NO: 1; a C, D, E, F, H, M, Q, S, T or W at a position corresponding to P14 of SEQ ID NO: 1; a Q or W at a position corresponding to V15 of SEQ ID NO: 1; a P at a position corresponding to R17 of SEQ ID NO: 1; a Y at a position corresponding to I18 of SEQ ID NO: 1; a W at a position corresponding to E22 of SEQ ID NO: 1; a D at a position corresponding to F34 of SEQ ID NO: 1; a C or T at a position corresponding to P36 of SEQ ID NO: 1; a D, E, F, G, S, or T at a position corresponding to Y37 of SEQ ID NO: 1; a L at a position corresponding to V71 of SEQ ID NO: 1; a R at a position corresponding to F87 of SEQ ID NO: 1; a D, P, or W at a position corresponding to H89 of SEQ ID NO: 1; a G at a position corresponding to P90 of SEQ ID NO: 1; a D, E, N, or Y at a position corresponding to Q91 of SEQ ID NO: 1; a P at a position corresponding to D92 of SEQ ID NO: 1; a C, D, E, F, G, H, I, M, N, Q, W, or Y at a position corresponding to P94 of SEQ ID NO: 1; a P and R at a position corresponding to I96 of SEQ ID NO: 1; a C, D, E, F, G, H, I, K, L, N, P, Q, S, T, V, or Y at a position corresponding to R97 of SEQ ID NO: 1; an E or Q at a position corresponding to R101 of SEQ ID NO: 1; a D or F at a position corresponding to I109 of SEQ ID NO: 1; a P at a position corresponding to Y110 of SEQ ID NO: 1; a G at a position corresponding to E111 of SEQ ID NO: 1; a D or E at a position corresponding to Y112 of SEQ ID NO: 1; a P at a position corresponding to D113 of SEQ ID NO: 1; an E, G, H, K, N, P, S, or Y at a position corresponding to I114 of SEQ ID NO: 1; a C, D, H, N, S, T, or Y at a position corresponding to P115 of SEQ ID NO: 1; a D, E, G, H, I, K, N, P, Q, R, or V at a position corresponding to F116 of SEQ ID NO: 1; a D, E, L, M, Q, or W at a position corresponding to A117 of SEQ ID NO: 1; a C, D, E, F, G, I, K, L, M, N, Q, S, T, V, W, or Y at a position corresponding to R119 of SEQ ID NO: 1; a P at a position corresponding to Y120 of SEQ ID NO: 1; a K, R, or W at a position corresponding to I122 of SEQ ID NO: 1; or a C, F, H, I, K, L, M, Q, R, S, T, V, W, or a Y at a position corresponding to D123 of SEQ ID NO: 1.

In some embodiments, the KOD polymerase variant further comprises one or more of the following substitutions: an A, C, F, G, I, L, M, P, V, or W at a position corresponding to Y7 of SEQ ID NO: 1; an A, C, F, H, K, S, W, or Y at a position corresponding to I16 of SEQ ID NO: 1; a D, E, F, H, I, K, L, M, N, Q, R, V, W, or Y at a position corresponding to P36 of SEQ ID NO: 1; a C, D, E, F, H, I, K, L, M, N, Q, R, W, or Y at a position corresponding to P90 of SEQ ID NO: 1; or an A, C, D, E, F, G, H, K, M, N, P, Q, R, S, T, W, or Y at a position corresponding to V93 of SEQ ID NO: 1.

In some embodiments, the KOD polymerase variant comprises one or more of the following substitutions: a D, E, H, L, N, V or W at a position corresponding to I2 of SEQ ID NO: 1; a D, H, I, S, or Y at a position corresponding to L3 of SEQ ID NO: 1; an I, L, M, or Y at a position corresponding to D4 of SEQ ID NO: 1; a D, E, F, G, H, L, W, or Y at a position corresponding to T5 of SEQ ID NO: 1; a L at a position corresponding to D6 of SEQ ID NO: 1; an A, C, D, E, F, G, H, I, K, L, M, N, P, Q, S, or W at a position corresponding to Y7 of SEQ ID NO: 1; a D, E, H, or T at a position corresponding to I8 of SEQ ID NO: 1; an A, C, D, E, F, H, I, L, N, R, or Y at a position corresponding to T9 of SEQ ID NO: 1; a P or Q at a position corresponding to E10 of SEQ ID NO: 1; a Q or T at a position corresponding to D11 of SEQ ID NO: 1; a C, Q, T, or V at a position corresponding to G12 of SEQ ID NO: 1; a G, H, T, V, or W at a position corresponding to K13 of SEQ ID NO: 1; a C, D, E, F, H, M, Q, S, T or W at a position corresponding to P14 of SEQ ID NO: 1; a Q or W at a position corresponding to V15 of SEQ ID NO: 1; an A, C, F, K, S, W, or Y at a position corresponding to I16 of SEQ ID NO: 1; a P at a position corresponding to R17 of SEQ ID NO: 1; a Y at a position corresponding to I18 of SEQ ID NO: 1; a D at a position corresponding to F34 of SEQ ID NO: 1; a D, E, F, H, I, K, L, M, N, Q, R, T, W, or Y at a position corresponding to P36 of SEQ ID NO: 1; a D, F, G, or T at a position corresponding to Y37 of SEQ ID NO: 1; a L at a position corresponding to V71 of SEQ ID NO: 1; a D or P at a position corresponding to H89 of SEQ ID NO: 1; a C, D, E, F, H, I, K, L, M, N, Q, R, W, or Y at a position corresponding to P90 of SEQ ID NO: 1; a Y at a position corresponding to Q91 of SEQ ID NO: 1; a P at a position corresponding to D92 of SEQ ID NO: 1; an A, C, D, E, F, G, H, K, M, N, P, Q, R, S, T, W, or Y at a position corresponding to V93 of SEQ ID NO: 1; a C, D, E, F, G, H, M, N, Q, W, or Y at a position corresponding to P94 of SEQ ID NO: 1; an A, C, D, E, F, G, H, I, K, L, M, N, P, Q, S, T, V, W, or Y at a position corresponding to R97 of SEQ ID NO: 1; an E or Q at a position corresponding to R101 of SEQ ID NO: 1; a D at a position corresponding to I109 of SEQ ID NO: 1; a G at a position corresponding to E111 of SEQ ID NO: 1; a G, H, P, Q, or Y at a position corresponding to I114 of SEQ ID NO: 1; a D, H, or T at a position corresponding to P115 of SEQ ID NO: 1; an A, D, E, G, H, K, P, Q, R, or V at a position corresponding to F116 of SEQ ID NO: 1; a D, L, M, Q, or W at a position corresponding to A117 of SEQ ID NO: 1; an A, C, E, F, H, I, K, L, M, Q, S, T, V, W, or Y at a position corresponding to R119 of SEQ ID NO: 1; a R at a position corresponding to I122 of SEQ ID NO: 1; or an A, F, H, K, L, M, Q, R, V, W, or a Y at a position corresponding to D123 of SEQ ID NO: 1.

In some embodiments, the KOD polymerase variant comprises one or more of the following substitutions: a D, E, F, H, K, L, N, T, V or W at a position corresponding to I2 of SEQ ID NO: 1; a D, H, I, S, T, or Y at a position corresponding to L3 of SEQ ID NO: 1; an I, L, M, P, or Y at a position corresponding to D4 of SEQ ID NO: 1; a D, E, F, G, H, L, W, or Y at a position corresponding to T5 of SEQ ID NO: 1; a L at a position corresponding to D6 of SEQ ID NO: 1; an A, C, D, E, F, G, H, I, K, L, M, N, P, Q, R, S, T, V, or W at a position corresponding to Y7 of SEQ ID NO: 1; a D, E, H, or T at a position corresponding to I8 of SEQ ID NO: 1; an A, C, D, E, F, H, N, R, or Y at a position corresponding to T9 of SEQ ID NO: 1; a Q at a position corresponding to E10 of SEQ ID NO: 1; a Q or T at a position corresponding to D11 of SEQ ID NO: 1; a C, Q, T, or V at a position corresponding to G12 of SEQ ID NO: 1; a G, H, T, V, or W at a position corresponding to K13 of SEQ ID NO: 1; a C, D, E, F, H, M, Q, S, T or W at a position corresponding to P14 of SEQ ID NO: 1; a Q or W at a position corresponding to V15 of SEQ ID NO: 1; an A, C, F, K, W, or Y at a position corresponding to I16 of SEQ ID NO: 1; a P at a position corresponding to R17 of SEQ ID NO: 1; a Y at a position corresponding to I18 of SEQ ID NO: 1; a D at a position corresponding to F34 of SEQ ID NO: 1; a D, E, F, H, I, K, L, M, N, Q, R, T, V, W, or Y at a position corresponding to P36 of SEQ ID NO: 1; an A, D, E, F, G, S, or T at a position corresponding to Y37 of SEQ ID NO: 1; a L at a position corresponding to V71 of SEQ ID NO: 1; a R at a position corresponding to F87 of SEQ ID NO: 1; a D, P, or W at a position corresponding to H89 of SEQ ID NO: 1; a C, D, E, F, G, H, I, K, L, M, N, Q, R, W, or Y at a position corresponding to P90 of SEQ ID NO: 1; a D, E, N, or Y at a position corresponding to Q91 of SEQ ID NO: 1; a P at a position corresponding to D92 of SEQ ID NO: 1; an A, C, D, E, F, G, H, K, M, N, P, Q, R, S, T, W, or Y at a position corresponding to V93 of SEQ ID NO: 1; a C, D, E, F, H, I, M, N, Q, W, or Y at a position corresponding to P94 of SEQ ID NO: 1; a P or R at a position corresponding to I96 of SEQ ID NO: 1; an A, C, D, E, F, G, H, I, K, L, M, N, P, Q, S, T, V, W, or Y at a position corresponding to R97 of SEQ ID NO: 1; a D or F at a position corresponding to I109 of SEQ ID NO: 1; a P at a position corresponding to Y110 of SEQ ID NO: 1; a G at a position corresponding to E111 of SEQ ID NO: 1; a D or E at a position corresponding to Y112 of SEQ ID NO: 1; a P at a position corresponding to D113 of SEQ ID NO: 1; an E, G, H, K, N, P, Q, S, or Y at a position corresponding to I114 of SEQ ID NO: 1; a C, D, F, H, N, S, T, or Y at a position corresponding to P115 of SEQ ID NO: 1; an A, D, E, H, I, K, N, P, Q, R, or V at a position corresponding to F116 of SEQ ID NO: 1; a D, E, L, M, Q, or W at a position corresponding to A117 of SEQ ID NO: 1; an A, C, D, E, F, G, H, I, K, L, M, N, Q, S, T, V, W, or Y at a position corresponding to R119 of SEQ ID NO: 1; a P at a position corresponding to Y120 of SEQ ID NO: 1; a K, R, or W at a position corresponding to I122 of SEQ ID NO: 1; or an A, C, F, H, I, K, L, M, Q, R, S, T, V, W, or a Y at a position corresponding to D123 of SEQ ID NO: 1.

In some embodiments, the KOD polymerase variant comprises one or more of the following substitutions: a D, H, L, or W at a position corresponding to I2 of SEQ ID NO: 1; a D, H, I, S, T, or Y at a position corresponding to L3 of SEQ ID NO: 1; an I, L, or Y at a position corresponding to D4 of SEQ ID NO: 1; a D, E, F, H, L, W, or Y at a position corresponding to T5 of SEQ ID NO: 1; a L at a position corresponding to D6 of SEQ ID NO: 1; an A, C, D, E, F, G, H, I, K, L, M, N, P, Q, R, S, T, V, or W at a position corresponding to Y7 of SEQ ID NO: 1; a D, E, H, or T at a position corresponding to I8 of SEQ ID NO: 1; an A, D, E, F, H, N, or R at a position corresponding to T9 of SEQ ID NO: 1; a P or Q at a position corresponding to E10 of SEQ ID NO: 1; a Q or T at a position corresponding to D11 of SEQ ID NO: 1; a C, Q, T, or V at a position corresponding to G12 of SEQ ID NO: 1; a G, T, V, or W at a position corresponding to K13 of SEQ ID NO: 1; a C, E, F, M, Q, S, or W at a position corresponding to P14 of SEQ ID NO: 1; a Q or W at a position corresponding to V15 of SEQ ID NO: 1; a C, F, H, or W at a position corresponding to I16 of SEQ ID NO: 1; a P at a position corresponding to R17 of SEQ ID NO: 1; a W at a position corresponding to E22 of SEQ ID NO: 1; a C, D, E, F, H, I, K, L, M, N, Q, R, T, V, W, or Y at a position corresponding to P36 of SEQ ID NO: 1; a D, E, F, G, S, or T at a position corresponding to Y37 of SEQ ID NO: 1; a L at a position corresponding to V71 of SEQ ID NO: 1; a R at a position corresponding to F87 of SEQ ID NO: 1; a D or P at a position corresponding to H89 of SEQ ID NO: 1; a C, D, E, F, G, H, I, K, L, M, N, Q, R, W, or Y at a position corresponding to P90 of SEQ ID NO: 1; a D or Y at a position corresponding to Q91 of SEQ ID NO: 1; a P at a position corresponding to D92 of SEQ ID NO: 1; an A, C, D, E, F, G, H, K, M, N, P, Q, R, S, T, W, or Y at a position corresponding to V93 of SEQ ID NO: 1; a D, F, H, I, N, W, or Y at a position corresponding to P94 of SEQ ID NO: 1; a P at a position corresponding to I96 of SEQ ID NO: 1; an A, C, D, E, F, G, H, I, K, L, M, N, P, Q, S, T, V, W, or Y at a position corresponding to R97 of SEQ ID NO: 1; a D at a position corresponding to I109 of SEQ ID NO: 1; a G at a position corresponding to E111 of SEQ ID NO: 1; a P at a position corresponding to D113 of SEQ ID NO: 1; an E, G, H, P, Q, or S at a position corresponding to I114 of SEQ ID NO: 1; a D, H, N, S, T, or Y at a position corresponding to P115 of SEQ ID NO: 1; a D, E, H, N, P, Q, R, or V at a position corresponding to F116 of SEQ ID NO: 1; a D, M, or W at a position corresponding to A117 of SEQ ID NO: 1; an A, C, D, E, F, G, I, K, L, M, N, Q, S, V, W, or Y at a position corresponding to R119 of SEQ ID NO: 1; a P at a position corresponding to Y120 of SEQ ID NO: 1; a W at a position corresponding to I122 of SEQ ID NO: 1; or an A, C, F, I, K, L, M, Q, R, S, V, W, or a Y at a position corresponding to D123 of SEQ ID NO: 1.

In some embodiments, the KOD polymerase variant comprises an amino acid sequence having at least 95% identity to SEQ ID NO: 1. In some embodiments, the KOD polymerase variant comprises an amino acid sequence of any one of SEQ ID NOs: 2-31, 34-35, 38, 40, 43, 45-48, 51-90, 99-103, 115, 119-130, 135, 138, 146-150, or 168-281.

In some embodiments, the KOD polymerase variant comprises one or more of the following substitutions: a D at a position corresponding to Y7 of SEQ ID NO: 1; an A, R, or S at a position corresponding to P36 of SEQ ID NO: 1; a S at a position corresponding to P90 of SEQ ID NO: 1; an I at a position corresponding to V93 of SEQ ID NO: 1; an A, F, H, P, or W at a position corresponding to R97 of SEQ ID NO: 1; and an E, M, P, Q, or Y at a position corresponding to R119 of SEQ ID NO: 1.

In some embodiments, the KOD polymerase variant comprises one or more of the following substitutions: a D at position Y7 of SEQ ID NO: 1; an A, R, or S at position P36 of SEQ ID NO: 1; a S at position P90 of SEQ ID NO: 1; an I at position V93 of SEQ ID NO: 1; an A, F, H, P, or W at position R97 of SEQ ID NO: 1; an E, M, P, Q, or Y at position R119 of SEQ ID NO: 1; and having at least 95% identity to SEQ ID NO: 1.

In some embodiments, the KOD polymerase variant comprises one or more of the following substitutions: a D at position Y7 of SEQ ID NO: 1; an A, R, or S at position P36 of SEQ ID NO: 1; a S at position P90 of SEQ ID NO: 1; an I at position V93 of SEQ ID NO: 1; an A, F, H, P, or W at position R97 of SEQ ID NO: 1; and an E, M, P, Q, or Y at position R119 of SEQ ID NO: 1.

In some embodiments, the KOD polymerase variant comprises a D at position Y7 of SEQ ID NO: 1. In some embodiments, the KOD polymerase variant comprises an A, R, or S at position P36 of SEQ ID NO: 1. In some embodiments, the KOD polymerase variant comprises a S at position P90 of SEQ ID NO: 1. In some embodiments, the KOD polymerase variant comprises an I at position V93 of SEQ ID NO: 1. In some embodiments, the KOD polymerase variant comprises an A, F, H, P, or W at position R97 of SEQ ID NO: 1. In some embodiments, the KOD polymerase variant comprises an E, M, P, Q, or Y at position R119 of SEQ ID NO: 1. In some embodiments, the KOD polymerase variant further comprises one or more of: a C, L, M or P at a position corresponding to Y7 of SEQ ID NO: 1; a F, H, L, M or W at a position corresponding to I16 of SEQ ID NO: 1; a H or W at a position corresponding to P36 of SEQ ID NO: 1; a F, I, W, or Y at a position corresponding to P90 of SEQ ID NO: 1; and an E, K, Q, or R at a position corresponding to V93 of SEQ ID NO: 1.

In some embodiments, the KOD polymerase variant comprises an amino acid sequence having at least 95% identity to any one of SEQ ID NOs: 741-1509 or 1511-3192. In some embodiments, the KOD polymerase variant comprises an amino acid sequence of any one of SEQ ID NOs: 741-1509 or 1511-3192.

In some embodiments, the KOD polymerase variant comprises one or more of the following substitutions: a D at a position corresponding to Y7 of SEQ ID NO: 1; an E at a position corresponding to P14 of SEQ ID NO: 1; a D at a position corresponding to Y37 of SEQ ID NO: 1; a D at a position corresponding to H89 of SEQ ID NO: 1; a D at a position corresponding to P94 of SEQ ID NO: 1; a F at a position corresponding to R97 of SEQ ID NO: 1; a P at a position corresponding to I114 of SEQ ID NO: 1; a P at a position corresponding to F116 of SEQ ID NO: 1; a M at a position corresponding to A117 of SEQ ID NO: 1; a Q at a position corresponding to R119 of SEQ ID NO: 1; and an I at a position corresponding to D123 of SEQ ID NO: 1.

In some embodiments, the KOD polymerase variant comprises one or more of the following substitutions: a D at position Y7 of SEQ ID NO: 1; an E at position P14 of SEQ ID NO: 1; a D at position Y37 of SEQ ID NO: 1; a D at position H89 of SEQ ID NO: 1; a D at position P94 of SEQ ID NO: 1; a F at position R97 of SEQ ID NO: 1; a P at position I114 of SEQ ID NO: 1; a P at position F116 of SEQ ID NO: 1; a M at position A117 of SEQ ID NO: 1; a Q at position R119 of SEQ ID NO: 1; an I at position D123 of SEQ ID NO: 1; and having at least 95% identity to SEQ ID NO: 1.

In some embodiments, the KOD polymerase variant comprises one or more of the following substitutions: a D at position Y7 of SEQ ID NO: 1; an E at position P14 of SEQ ID NO: 1; a D at position Y37 of SEQ ID NO: 1; a D at position H89 of SEQ ID NO: 1; a D at position P94 of SEQ ID NO: 1; a F at position R97 of SEQ ID NO: 1; a P at position I114 of SEQ ID NO: 1; a P at position F116 of SEQ ID NO: 1; a M at position A117 of SEQ ID NO: 1; a Q at position R119 of SEQ ID NO: 1; and an I at position D123 of SEQ ID NO: 1.

In some embodiments, the KOD polymerase variant comprises a D at position Y7 of SEQ ID NO: 1. In some embodiments, the KOD polymerase variant comprises an E at position P14 of SEQ ID NO: 1. In some embodiments, the KOD polymerase variant comprises a D at position Y37 of SEQ ID NO: 1. In some embodiments, the KOD polymerase variant comprises a D at position H89 of SEQ ID NO: 1. In some embodiments, the KOD polymerase variant comprises a D at position P94 of SEQ ID NO: 1. In some embodiments, the KOD polymerase variant comprises a F at position R97 of SEQ ID NO: 1. In some embodiments, the KOD polymerase variant comprises a P at position I114 of SEQ ID NO: 1. In some embodiments, the KOD polymerase variant comprises a P at position F116 of SEQ ID NO: 1. In some embodiments, the KOD polymerase variant comprises a M at position A117 of SEQ ID NO: 1. In some embodiments, the KOD polymerase variant comprises a Q at position R119 of SEQ ID NO: 1. In some embodiments, the KOD polymerase variant comprises an I at position D123 of SEQ ID NO: 1.

In some embodiments, the KOD polymerase variant further comprises: a W at a position corresponding to I16 of SEQ ID NO: 1; a H at a position corresponding to P36 of SEQ ID NO: 1; a F at a position corresponding to P90 of SEQ ID NO: 1; and/or a K at a position corresponding to V93 of SEQ ID NO: 1.

In some embodiments, the KOD polymerase variant comprises an amino acid sequence having at least 95% identity to any one of SEQ ID NOs: 3193-3418. In some embodiments, the KOD polymerase variant comprises an amino acid sequence of any one of SEQ ID NOs: 3193-3418.

In some embodiments, the KOD polymerase variant comprising one or more of the following substitutions: a G or K at a position corresponding to P90 of SEQ ID NO: 1; an A, C, D, E, F, G, M, N, P, S, T, W, or Y at a position corresponding to V93 of SEQ ID NO: 1; an A, D, E, G, H, I, K, N, P, Q, R, or V at a position corresponding to F116 of SEQ ID NO: 1; a D, E, L, M, Q, or W at a position corresponding to A117 of SEQ ID NO: 1; and an A, C, D, E, G, H, I, K, L, M, N, P, Q, R, S, T, V, or W at a position corresponding to F214 of SEQ ID NO: 1.

In some embodiments, the KOD polymerase variant comprises one or more of the following substitutions: a G or K at position P90 of SEQ ID NO: 1; an A, C, D, E, F, G, M, N, P, S, T, W, or Y at position V93 of SEQ ID NO: 1; an A, D, E, G, H, I, K, N, P, Q, R, or V at position F116 of SEQ ID NO: 1; and a D, E, L, M, Q, or W at position A117 of SEQ ID NO: 1.

In some embodiments, the KOD polymerase variant further comprises one or more of the following: an A, C, F, H, K, S, W, or Y at a position corresponding to I16 of SEQ ID NO: 1; a H, K, Q, or R at a position corresponding to V93 of SEQ ID NO: 1; and a C, D, E, F, H, I, L, M, N, Q, R, W, or Y at position corresponding to P90 of SEQ ID NO: 1.

In some aspects, this disclosure provides a Thermococcus kodakarensis (KOD) polymerase variant, comprising one or more of the following substitutions: a D at position Y7 of SEQ ID NO: 1; an E at position P14 of SEQ ID NO: 1; a L or M at position I16 of SEQ ID NO: 1; an A or S at position P36 of SEQ ID NO: 1; a D at position Y37 of SEQ ID NO: 1; a D at position H89 of SEQ ID NO: 1; a S at position P90 of SEQ ID NO: 1; an I at position V93 of SEQ ID NO: 1; a D at position P94 of SEQ ID NO: 1; an A, F, H, P, or W at position R97 of SEQ ID NO: 1; a P at position I114 of SEQ ID NO: 1; a P at position F116 of SEQ ID NO: 1; a M at position A117 of SEQ ID NO: 1; an E, M, P, Q, or Y at position R119 of SEQ ID NO: 1; or an I at position D123 of SEQ ID NO: 1.

In some embodiments, the KOD polymerase variant further comprises one or more of the following substitutions: a C, L, M, or P at position Y7 of SEQ ID NO: 1; a F, H, or W at position I16 of SEQ ID NO: 1; a H, R, or W at position P36 of SEQ ID NO: 1; a F, I, W, or Y at position P90 of SEQ ID NO:1; or an E, K, Q, or R at position V93 of SEQ ID NO: 1.

In some embodiments, the KOD polymerase variant comprises an I at position V93 of SEQ ID NO: 1. In some embodiments, the KOD polymerase variant comprises a P at position F116 of SEQ ID NO: 1. In some embodiments, the KOD polymerase variant comprises a M at position A117 of SEQ ID NO: 1. In some embodiments, the KOD polymerase variant comprises a F at position P90 of SEQ ID NO: 1.

In some embodiments, the KOD polymerase variant comprises one or more of the following substitutions: a L or M at position Y7 of SEQ ID NO: 1; a F, L, or W at position I16 of SEQ ID NO: 1; a H at position P36 of SEQ ID NO: 1; a F at position P90 of SEQ ID NO: 1; an I or K at position V93 of SEQ ID NO: 1; a D at position P94 of SEQ ID NO: 1; an A or F at position R97 of SEQ ID NO: 1; a P at position F116 of SEQ ID NO: 1; a M at position A117 of SEQ ID NO: 1; or an I at position D123 of SEQ ID NO: 1.

In some embodiments, the KOD polymerase variant comprises two or more of the substitutions. In some embodiments, the KOD polymerase variant comprises three or more of the substitutions. In some embodiments, the KOD polymerase variant comprises four or more of the substitutions. In some embodiments, the KOD polymerase variant comprises five or more of the substitutions.

In some embodiments, the KOD polymerase variant comprises: a M at position Y7 of SEQ ID NO: 1; and a L at position I16 of SEQ ID NO: 1.

In some embodiments, the KOD polymerase variant comprises: a W at position I16 of SEQ ID NO: 1; and an I at position V93 of SEQ ID NO: 1.

In some embodiments, the KOD polymerase variant comprises: a W at position I16 of SEQ ID NO: 1; and a P at position F116 of SEQ ID NO: 1.

In some embodiments, the KOD polymerase variant comprises: a L at position Y7 of SEQ ID NO: 1; a F at position I16 of SEQ ID NO: 1; and an I at position V93 of SEQ ID NO: 1.

In some embodiments, the KOD polymerase variant comprises: a F at position I16 of SEQ ID NO: 1; a F at position P90 of SEQ ID NO: 1; and an I at position V93 of SEQ ID NO: 1.

In some embodiments, the KOD polymerase variant comprises: a F at position I16 of SEQ ID NO: 1; an I at position P90 of SEQ ID NO: 1; and an I at position V93 of SEQ ID NO: 1.

In some embodiments, the KOD polymerase variant comprises: a W at position I16 of SEQ ID NO: 1; a P at position F116 of SEQ ID NO: 1; and an M at position A117 of SEQ ID NO: 1.

In some embodiments, the KOD polymerase variant comprises: a F at position P90 of SEQ ID NO: 1; a P at position F116 of SEQ ID NO:1; and a M at position A117 of SEQ ID NO: 1.

In some embodiments, the KOD polymerase variant comprises: a L and position Y7 of SEQ ID NO: 1; a F at position I16 of SEQ ID NO: 1; a F at position P90 of SEQ ID NO: 1; and an I at position V93 of SEQ ID NO: 1.

In some embodiments, the KOD polymerase variant comprises: a M and position Y7 of SEQ ID NO: 1; a L at position I16 of SEQ ID NO: 1; a W at position P36 of SEQ ID NO: 1; and an I at position V93 of SEQ ID NO: 1.

In some embodiments, the KOD polymerase variant comprises: a W and position I16 of SEQ ID NO: 1; a H at position P36 of SEQ ID NO: 1; a P at position F116 of SEQ ID NO: 1; and an M at position A117 of SEQ ID NO: 1.

In some embodiments, the KOD polymerase variant an amino acid sequence having at least 95% identity to SEQ ID NO: 1.

In some aspects, this disclosure provides a Thermococcus kodakarensis (KOD) polymerase variant, comprising one or more of the following substitutions: an asparagine (N) at a position corresponding to leucine 138 (L138) of SEQ ID NO: 1; a glutamate (E) or glutamine (Q) at a position corresponding to alanine 139 (A139) of SEQ ID NO:1; an arginine (R) at a position corresponding to phenylalanine 140 (F140) of SEQ ID NO: 1; a cysteine (C), glycine (G), histidine (H), lysine (K), N, proline (P), Q, serine (S), threonine (T), or valine (V) at a position corresponding to aspartate 141 (D141) of SEQ ID NO: 1; a F, G, H, K, P, tryptophan (W), or tyrosine (Y) at a position corresponding to isoleucine 142 (I142) of SEQ ID NO: 1; a C, F, G, H, I, K, L, methionine (M), N, P, Q, R, S, T, V, W, or Y at a position corresponding to E143 of SEQ ID NO: 1; an A, D, E, F, G, H, K, L, M, P, Q, R, W, or Y at a position corresponding to T144 of SEQ ID NO: 1; a G at a position corresponding to L145 of SEQ ID NO: 1; a D or E at a position corresponding to Y146 of SEQ ID NO: 1; a L at a position corresponding to G149 of SEQ ID NO: 1; an A, D, E, N, or S at a position corresponding to F152 of SEQ ID NO: 1; a P or Y at a position corresponding to E154 of SEQ ID NO: 1; an I or V at a position corresponding to G155 of SEQ ID NO: 1; an E, F, R, W, or Y at a position corresponding to I157 of SEQ ID NO: 1; a D, H, K, Q, R, W, or Y at a position corresponding to L158 of SEQ ID NO: 1; an E, G, P, or R at a position corresponding to M159 of SEQ ID NO: 1; a F at a position corresponding to I160 of SEQ ID NO: 1; a D, E, F, H, K, L, M, N, P, R, T, W, or Y at a position corresponding to S161 of SEQ ID NO: 1; a P at a position corresponding to Y162 of SEQ ID NO: 1; a C, E, F, G, N, or T at a position corresponding to A168 of SEQ ID NO: 1; an A, G, H, R, S, W, or Y at a position corresponding to V170 of SEQ ID NO: 1; a R at a position corresponding to T172 of SEQ ID NO: 1; a W at a position corresponding to E187 of SEQ ID NO: 1; a F or Y at a position corresponding to I191 of SEQ ID NO: 1; a G at a position corresponding to F194 of SEQ ID NO: 1; a F at a position corresponding to K199 of SEQ ID NO: 1; an E, F, H, I, K, N, Q, R, V, W, or Y at a position corresponding to T208 of SEQ ID NO: 1; an A, C, E, G, H, I, L, M, N, P, S, T, V, or W at a position corresponding to Y209 of SEQ ID NO: 1; a C, E, G, M, P, Q, S, or T at a position corresponding to N210 of SEQ ID NO: 1; a C, D, E, F, H, I, K, L, M, N, P, Q, R, S, T, V, W or Y at a position corresponding to G211 of SEQ ID NO: 1; a F, I, K, L, M, P, Q, R, V, W, or Y at a position corresponding to D212 of SEQ ID NO: 1; a D or E at a position corresponding to N213 of SEQ ID NO: 1; an A, C, D, E, G, H, I, K, L, M, N, P, Q, R, S, T, V, or W at a position corresponding to F214 of SEQ ID NO: 1; an A, C, F, G, H, I, K, L, M, N, P, Q, R, S, T, V, W, or Y at a position corresponding to D215 of SEQ ID NO: 1; an A or D at a position corresponding to F216 of SEQ ID NO: 1; an A, D, E, G, L, P, S, T, or W at a position corresponding to Y218 of SEQ ID NO: 1; a P, R, or V at a position corresponding to L219 of SEQ ID NO: 1; an I or V at a position corresponding to K220 of SEQ ID NO: 1; an A, E, or P at a position corresponding to C223 of SEQ ID NO: 1; a W or Y at a position corresponding to K240 of SEQ ID NO: 1; a D at a position corresponding to A249 of SEQ ID NO: 1; a L at a position corresponding to V250 of SEQ ID NO: 1; a F, G, N, or R at a position corresponding to E251 of SEQ ID NO: 1; a C, E, M, T, or W at a position corresponding to V252 of SEQ ID NO: 1; a H, N, or R at a position corresponding to G254 of SEQ ID NO: 1; a F, W, or Y at a position corresponding to H257 of SEQ ID NO: 1; an A, C, F, G, H, I, K, L, M, N, P, Q, R, S, T, V, W, or Y at a position corresponding to D259 of SEQ ID NO: 1; a W at a position corresponding to L260 of SEQ ID NO: 1; a D, E, or F at a position corresponding to Y261 of SEQ ID NO: 1; a D or P at a position corresponding to Y273 of SEQ ID NO: 1; a D at a position corresponding to T274 of SEQ ID NO: 1; a H, P, R, or S at a position corresponding to L275 of SEQ ID NO: 1; an E or P at a position corresponding to A277 of SEQ ID NO: 1; a F at a position corresponding to V278 of SEQ ID NO: 1; a M at a position corresponding to E280 of SEQ ID NO: 1; a W at a position corresponding to A281 of SEQ ID NO: 1; a D, E, F, I, M, P, T, or V at a position corresponding to K287 of SEQ ID NO: 1; an A, D, E, F, G, H, I, L, M, P, Q, R, S, T, V, W, or Y at a position corresponding to K289 of SEQ ID NO: 1; an A, D, E, G, H, K, N, P, Q, R, S, or T at a position corresponding to V290 of SEQ ID NO: 1; a D, E, N, Q, or T at a position corresponding to A292 of SEQ ID NO: 1; a K, P, or R at a position corresponding to E294 of SEQ ID NO: 1; an A, C, D, E, G, H, K, N, P, Q, R, S, T, W, or Y at a position corresponding to I295 of SEQ ID NO: 1; a D or P at a position corresponding to A298 of SEQ ID NO: 1; a D, E, G, H, I, K, L, M, N, P, Q, R, T, or V at a position corresponding to W299 of SEQ ID NO: 1; a P at a position corresponding to E300 of SEQ ID NO: 1; a P at a position corresponding to T301 of SEQ ID NO: 1; a P at a position corresponding to G302 of SEQ ID NO: 1; a H at a position corresponding to L305 of SEQ ID NO: 1; a D, F, H, K, L, N, Q, R, W, or Y at a position corresponding to V308 of SEQ ID NO: 1; a D, P, or R at a position corresponding to A309 of SEQ ID NO: 1; an A, C, D, E, G, H, I, K, L, M, N, P, Q, R, S, T, V, or W at a position corresponding to Y311 of SEQ ID NO: 1; a D, E, F, H, I, K, L, M, N, P, Q, R, V, W, or Y at a position corresponding to S312 of SEQ ID NO: 1; a P or Q at a position corresponding to M313 of SEQ ID NO: 1; a P at a position corresponding to E314 of SEQ ID NO: 1; an A, C, F, G, I, K, L, M, N, P, Q, R, S, T, V, W, or Y at a position corresponding to D315 of SEQ ID NO: 1; a D, E, F, H, M, N, Q, R, W, or Y at a position corresponding to A316 of SEQ ID NO: 1; a P at a position corresponding to K317 of SEQ ID NO: 1; a P or R at a position corresponding to V318 of SEQ ID NO: 1; or a D, I, K, L, M, Q, R, or V at a position corresponding to T319 of SEQ ID NO: 1.

In some embodiments, the KOD polymerase variant further comprises one or more of the following substitutions: an A at a position corresponding to D141 of SEQ ID NO: 1; a R at a position corresponding to I142 of SEQ ID NO: 1; an A at a position corresponding to E143 of SEQ ID NO: 1; a D at a position corresponding to N210 of SEQ ID NO: 1; or a F at a position corresponding to Y311 of SEQ ID NO: 1.

In some embodiments, the KOD polymerase variant comprises one or more of the following substitutions: a N at a position corresponding to L138 of SEQ ID NO: 1; an E or Q at a position corresponding to A139 of SEQ ID NO:1; a R at a position corresponding to F140 of SEQ ID NO: 1; an A, C, G, H, K, N, P, Q, S, T, or V at a position corresponding to D141 of SEQ ID NO: 1; a H, K, P, R, W, or Y at a position corresponding to I142 of SEQ ID NO: 1; an A, C, F, G, H, I, K, L, M, N, P, Q, R, S, T, V, W, or Y at a position corresponding to E143 of SEQ ID NO: 1; a D, E, F, H, K, L, M, P, Q, R, W, or Y at a position corresponding to T144 of SEQ ID NO: 1; an E at a position corresponding to Y146 of SEQ ID NO: 1; a D at a position corresponding to F152 of SEQ ID NO: 1; a Y at a position corresponding to E154 of SEQ ID NO: 1; a F, R, W, or Y at a position corresponding to I157 of SEQ ID NO: 1; a D, H, K, Q, R, or W at a position corresponding to L158 of SEQ ID NO: 1; a P or R at a position corresponding to M159 of SEQ ID NO: 1; a F at a position corresponding to I160 of SEQ ID NO: 1; a D, F, H, K, L, M, N, R, T, or W at a position corresponding to S161 of SEQ ID NO: 1; a P at a position corresponding to Y162 of SEQ ID NO: 1; an E, F, or N at a position corresponding to A168 of SEQ ID NO: 1; a G, H, R, W, or Y at a position corresponding to V170 of SEQ ID NO: 1; a G at a position corresponding to F194 of SEQ ID NO: 1; an E, F, H, K, N, Q, R, V, W, or Y at a position corresponding to T208 of SEQ ID NO: 1; an E, G, I, L, N, P, T, V, or W at a position corresponding to Y209 of SEQ ID NO: 1; a D, E, M, P, Q, or T at a position corresponding to N210 of SEQ ID NO: 1; a C, D, E, F, H, I, K, L, M, N, P, Q, R, S, T, V, W or Y at a position corresponding to G211 of SEQ ID NO: 1; a F, K, L, Q, R, V, W, or Y at a position corresponding to D212 of SEQ ID NO: 1; a D or E at a position corresponding to N213 of SEQ ID NO: 1; an A, C, D, E, G, H, I, K, L, M, N, P, Q, R, S, T, V, or W at a position corresponding to F214 of SEQ ID NO: 1; an A, C, F, G, H, I, K, L, M, N, P, Q, R, S, T, V, W, or Y at a position corresponding to D215 of SEQ ID NO: 1; an A, D, G, L, P, or T at a position corresponding to Y218 of SEQ ID NO: 1; a R at a position corresponding to L219 of SEQ ID NO: 1; an I at a position corresponding to K220 of SEQ ID NO: 1; an A or E at a position corresponding to C223 of SEQ ID NO: 1; a W or Y at a position corresponding to K240 of SEQ ID NO: 1; a L at a position corresponding to V250 of SEQ ID NO: 1; a R at a position corresponding to E251 of SEQ ID NO: 1; a C, E, M, or T at a position corresponding to V252 of SEQ ID NO: 1; a H, N, or R at a position corresponding to G254 of SEQ ID NO: 1; a F or Y at a position corresponding to H257 of SEQ ID NO: 1; an A, C, F, G, H, I, L, M, N, P, Q, R, S, T, V, W, or Y at a position corresponding to D259 of SEQ ID NO: 1; a D or E at a position corresponding to Y261 of SEQ ID NO: 1; a D at a position corresponding to T274 of SEQ ID NO: 1; a H, P, R, or S at a position corresponding to L275 of SEQ ID NO: 1; a P at a position corresponding to A277 of SEQ ID NO: 1; a D, E, F, I, P, T, or V at a position corresponding to K287 of SEQ ID NO: 1; an A, D, E, F, G, H, I, M, P, Q, T, V, W, or Y at a position corresponding to K289 of SEQ ID NO: 1; a D, E, G, H, K, N, P, R, or S at a position corresponding to V290 of SEQ ID NO: 1; a D, E, or N at a position corresponding to A292 of SEQ ID NO: 1; a P at a position corresponding to E294 of SEQ ID NO: 1; an A, D, E, G, H, K, N, P, Q, R, S, T, W, or Y at a position corresponding to I295 of SEQ ID NO: 1; a P at a position corresponding to A298 of SEQ ID NO: 1; a D, E, G, I, K, L, N, P, Q, R, T, or V at a position corresponding to W299 of SEQ ID NO: 1; a P at a position corresponding to E300 of SEQ ID NO: 1; a P at a position corresponding to T301 of SEQ ID NO: 1; a P at a position corresponding to G302 of SEQ ID NO: 1; a H at a position corresponding to L305 of SEQ ID NO: 1; a D, F, K, N, Q, R, W, or Y at a position corresponding to V308 of SEQ ID NO: 1; a P or R at a position corresponding to A309 of SEQ ID NO: 1; an A, C, D, E, F, G, H, I, K, L, M, N, P, Q, R, S, T, V, or W at a position corresponding to Y311 of SEQ ID NO: 1; a D, E, F, H, I, K, L, M, N, P, Q, R, V, W, or Y at a position corresponding to S312 of SEQ ID NO: 1; a P at a position corresponding to M313 of SEQ ID NO: 1; a P at a position corresponding to E314 of SEQ ID NO: 1; an A, C, F, G, I, K, L, M, N, P, Q, R, S, T, V, W, or Y at a position corresponding to D315 of SEQ ID NO: 1; a D, E, F, H, N, Q, W, or Y at a position corresponding to A316 of SEQ ID NO: 1; a P at a position corresponding to K317 of SEQ ID NO: 1; a P at a position corresponding to V318 of SEQ ID NO: 1; or a D, K, M, or R at a position corresponding to T319 of SEQ ID NO: 1.

In some embodiments, the KOD polymerase variant comprises one or more of the following substitutions: a N at a position corresponding to L138 of SEQ ID NO: 1; an E or Q at a position corresponding to A139 of SEQ ID NO:1; a R at a position corresponding to F140 of SEQ ID NO: 1; an A, C, G, H, K, N, P, Q, S, T, or V at a position corresponding to D141 of SEQ ID NO: 1; a F, G, H, K, P, R, W, or Y at a position corresponding to I142 of SEQ ID NO: 1; an A, C, F, G, H, I, K, L, M, N, P, Q, R, S, T, V, W, or Y at a position corresponding to E143 of SEQ ID NO: 1; an A, D, E, F, G, H, K, L, M, P, Q, R, W, or Y at a position corresponding to T144 of SEQ ID NO: 1; a G at a position corresponding to L145 of SEQ ID NO: 1; a D or E at a position corresponding to Y146 of SEQ ID NO: 1; an A, D, E, N, or S at a position corresponding to F152 of SEQ ID NO: 1; a P at a position corresponding to E154 of SEQ ID NO: 1; an I or V at a position corresponding to G155 of SEQ ID NO: 1; an E, F, R, W, or Y at a position corresponding to I157 of SEQ ID NO: 1; a D, H, K, Q, R, W, or Y at a position corresponding to L158 of SEQ ID NO: 1; an E, G, P, or R at a position corresponding to M159 of SEQ ID NO: 1; a D, E, F, H, K, L, M, N, P, R, T, W, or Y at a position corresponding to S161 of SEQ ID NO: 1; a P at a position corresponding to Y162 of SEQ ID NO: 1; a C, E, F, G, N, or T at a position corresponding to A168 of SEQ ID NO: 1; an A, G, H, R, S, W, or Y at a position corresponding to V170 of SEQ ID NO: 1; a R at a position corresponding to T172 of SEQ ID NO: 1; a W at a position corresponding to E187 of SEQ ID NO: 1; a F or Y at a position corresponding to I191 of SEQ ID NO: 1; a G at a position corresponding to F194 of SEQ ID NO: 1; a F at a position corresponding to K199 of SEQ ID NO: 1; an E, F, H, I, K, N, Q, R, V, W, or Y at a position corresponding to T208 of SEQ ID NO: 1; an A, C, E, G, H, I, L, M, N, P, S, T, V, or W at a position corresponding to Y209 of SEQ ID NO: 1; a C, D, E, G, M, P, Q, S, or T at a position corresponding to N210 of SEQ ID NO: 1; a C, D, E, F, H, I, K, L, M, N, P, Q, R, S, T, V, W or Y at a position corresponding to G211 of SEQ ID NO: 1; a F, I, K, L, M, P, Q, R, V, W, or Y at a position corresponding to D212 of SEQ ID NO: 1; a D or E at a position corresponding to N213 of SEQ ID NO: 1; an A, C, D, E, G, H, I, K, L, M, N, P, Q, R, S, T, V, or W at a position corresponding to F214 of SEQ ID NO: 1; an A, C, F, G, H, I, K, L, M, N, P, Q, R, S, T, V, W, or Y at a position corresponding to D215 of SEQ ID NO: 1; an A or D at a position corresponding to F216 of SEQ ID NO: 1; a D, E, G, L, P, S, T, or W at a position corresponding to Y218 of SEQ ID NO: 1; a P, R, or V at a position corresponding to L219 of SEQ ID NO: 1; an I or V at a position corresponding to K220 of SEQ ID NO: 1; an A, E, or P at a position corresponding to C223 of SEQ ID NO: 1; a W or Y at a position corresponding to K240 of SEQ ID NO: 1; a D at a position corresponding to A249 of SEQ ID NO: 1; a L at a position corresponding to V250 of SEQ ID NO: 1; a F, G, N, or R at a position corresponding to E251 of SEQ ID NO: 1; a C, E, M, or W at a position corresponding to V252 of SEQ ID NO: 1; a H, N, or R at a position corresponding to G254 of SEQ ID NO: 1; a F, W, or Y at a position corresponding to H257 of SEQ ID NO: 1; an A, C, F, G, H, K, L, M, N, P, Q, R, S, T, V, W, or Y at a position corresponding to D259 of SEQ ID NO: 1; a W at a position corresponding to L260 of SEQ ID NO: 1; a D or E at a position corresponding to Y261 of SEQ ID NO: 1; a D or P at a position corresponding to Y273 of SEQ ID NO: 1; a D at a position corresponding to T274 of SEQ ID NO: 1; a H or R at a position corresponding to L275 of SEQ ID NO: 1; an E or P at a position corresponding to A277 of SEQ ID NO: 1; a F at a position corresponding to V278 of SEQ ID NO: 1; a W at a position corresponding to A281 of SEQ ID NO: 1; a D, E, F, I, M, P, T, or V at a position corresponding to K287 of SEQ ID NO: 1; an A, D, F, G, H, I, M, P, Q, R, S, T, V, W, or Y at a position corresponding to K289 of SEQ ID NO: 1; an A, D, E, G, H, K, N, P, Q, R, S, or T at a position corresponding to V290 of SEQ ID NO: 1; a D, E, N, Q, or T at a position corresponding to A292 of SEQ ID NO: 1; a K, P, or R at a position corresponding to E294 of SEQ ID NO: 1; an A, C, D, E, G, H, K, N, P, Q, R, S, T, W, or Y at a position corresponding to I295 of SEQ ID NO: 1; a D or P at a position corresponding to A298 of SEQ ID NO: 1; a D, E, G, H, I, K, L, M, N, P, Q, R, T, or V at a position corresponding to W299 of SEQ ID NO: 1; a P at a position corresponding to E300 of SEQ ID NO: 1; a P at a position corresponding to T301 of SEQ ID NO: 1; a P at a position corresponding to G302 of SEQ ID NO: 1; a H at a position corresponding to L305 of SEQ ID NO: 1; a D, F, H, K, L, N, Q, R, W, or Y at a position corresponding to V308 of SEQ ID NO: 1; a D, P, or R at a position corresponding to A309 of SEQ ID NO: 1; an A, C, D, E, F, G, H, I, K, L, M, N, P, Q, R, S, T, V, or W at a position corresponding to Y311 of SEQ ID NO: 1; a D, E, F, H, I, K, L, M, N, P, Q, R, V, W, or Y at a position corresponding to S312 of SEQ ID NO: 1; a P or Q at a position corresponding to M313 of SEQ ID NO: 1; a P at a position corresponding to E314 of SEQ ID NO: 1; an A, C, F, G, I, L, M, N, P, Q, R, S, T, V, W, or Y at a position corresponding to D315 of SEQ ID NO: 1; a D, E, F, H, M, N, Q, R, W, or Y at a position corresponding to A316 of SEQ ID NO: 1; a P at a position corresponding to K317 of SEQ ID NO: 1; a P or R at a position corresponding to V318 of SEQ ID NO: 1; or a D, I, K, L, M, Q, R, or V at a position corresponding to T319 of SEQ ID NO: 1.

In some embodiments, the KOD polymerase variant comprises one or more of the following substitutions: a H at a position corresponding to I142 of SEQ ID NO: 1; a V at a position corresponding to E143 of SEQ ID NO: 1; a L at a position corresponding to G149 of SEQ ID NO: 1; a Y at a position corresponding to E154 of SEQ ID NO: 1; an E or F at a position corresponding to A168 of SEQ ID NO: 1; a F at a position corresponding to K199 of SEQ ID NO: 1; a V at a position corresponding to T208 of SEQ ID NO: 1; a H, P, or Q at a position corresponding to G211 of SEQ ID NO: 1; an A, F, M, or V at a position corresponding to D215 of SEQ ID NO: 1; a W at a position corresponding to Y218 of SEQ ID NO: 1; a W at a position corresponding to K240 of SEQ ID NO: 1; a F at a position corresponding to E251 of SEQ ID NO: 1; an E at a position corresponding to V252 of SEQ ID NO: 1; a R at a position corresponding to G254 of SEQ ID NO: 1; a F at a position corresponding to Y261 of SEQ ID NO: 1; a M at a position corresponding to E280 of SEQ ID NO: 1; a T at a position corresponding to K287 of SEQ ID NO: 1; a H at a position corresponding to K289 of SEQ ID NO: 1; a K at a position corresponding to serine 312 (S312) of SEQ ID NO: 1; or a G or S at a position corresponding to D315 of SEQ ID NO: 1.

In some embodiments, the KOD polymerase variant comprises an amino acid sequence having at least 95% identity to SEQ ID NO: 1. In some embodiments, the KOD polymerase variant comprises an amino acid sequence of any one of SEQ ID NO: 282-285, 287-301, 303-425, 427-669, or 671-740. In some embodiments, the KOD polymerase variant comprises any of the above substitutions.

In some embodiments, the KOD polymerase variant further comprises an Sso7d domain.

In some embodiments, the KOD polymerase variant comprises an Sso7d domain operably linked to the C-terminal end of the KOD polymerase variant.

In some aspects, the disclosure provides a method for amplifying a DNA template wherein the DNA template comprises one or more uracil and/or inosine nucleotides, the method comprising amplifying the DNA template using a KOD polymerase variant of the disclosure.

In some aspects, the disclosure provides a method for amplifying a DNA template to create a synthesized DNA strand in which synthesized DNA strand comprises one or more uracil residues, the method comprising inserting one or more uracil nucleotides into the synthesized DNA strand using a KOD polymerase variant of the disclosure.

In some aspects, the disclosure provides a method for amplifying a DNA template to create a synthesized DNA strand in which the DNA template comprises one or more uracil and/or inosine nucleotides and/or the synthesized DNA strand comprises one or more uracil residues, the method comprising amplifying the DNA template and/or inserting one or more uracil nucleotides into the synthesized DNA strand using a KOD polymerase variant of the disclosure.

In some aspects, the disclosure provides a method for amplifying a DNA template to create a synthesized DNA strand in which the DNA template comprises one or more uracil and/or inosine nucleotides and/or the synthesized DNA strand comprises one or more uracil residues, the method comprising amplifying the DNA template and/or inserting one or more uracil nucleotides into the synthesized DNA strand using a KOD polymerase variant of the disclosure.

In some aspects, the disclosure provides a Thermococcus kodakarensis (KOD) polymerase variant, comprising: a W at a position corresponding to I16 of SEQ ID NO: 1; an F at a position corresponding to P90 of SEQ ID NO: 1; a W at a position corresponding to P90 of SEQ ID NO: 1; a P at a position corresponding to F116 of SEQ ID NO: 1; an M at a position corresponding to A117 of SEQ ID NO: 1; a Q at a position corresponding to R119 of SEQ ID NO: 1; an I at a position corresponding to D123 of SEQ ID NO: 1; an M at a position corresponding to D215 of SEQ ID NO: 1; a W at a position corresponding to I16 of SEQ ID NO: 1 and a P at a position corresponding to F116 of SEQ ID NO: 1; an F at a position corresponding to P90 of SEQ ID NO: 1, a P at a position corresponding to F116 of SEQ ID NO: 1, and an M at a position corresponding to A117 of SEQ ID NO: 1; a W at a position corresponding to I16 of SEQ ID NO: 1, an H at a position corresponding to P36 of SEQ ID NO: 1, and a P at a position corresponding to F116 of SEQ ID NO: 1; an L at a position corresponding to Y7 of SEQ ID NO: 1, an F at a position corresponding to I16 of SEQ ID NO: 1, and an I at a position corresponding to V93 of SEQ ID NO: 1; or an M at a position corresponding to Y7 of SEQ ID NO: 1 and an L at a position corresponding to I16 of SEQ ID NO: 1.

### BRIEF DESCRIPTION OF DRAWINGS

FIGS. 1A-IC show heatmaps indicating the fitness_combined score of Thermococcus kodakarensis (KOD) polymerase variants with a single amino acid substitution in the N-terminal domain of a KOD polymerase relative to the wildtype sequence (SEQ ID NO: 1). FIG. 1A shows fitness_combined scores indicative of the ability of a KOD variant to tolerate dI and dU in the template strand (by showing improved dI/dU primer tolerance). FIG. 1B shows fitness_combined scores indicative of the ability of a KOD variant to tolerate dU in the template strand (by showing improved dU primer tolerance). FIG. 1C shows fitness_combined scores indicative of the ability of a KOD variant to incorporate dUTP into an extension product and to use extension products containing uracil as templates. White squares are positions excluded from analysis. Diagonal striped squares are positions with low counts, excluded due to low confidence in performance predictability.

FIGS. 2A-2C show heatmaps indicating the fitness_combined score of Thermococcus kodakarensis (KOD) polymerase variants with a single amino acid substitution in the exonuclease domain of a KOD polymerase relative to the wildtype sequence (SEQ ID NO: 1). FIG. 2A shows fitness_combined scores indicative of the ability of a KOD variant to tolerate dI and dU in the template strand (by showing improved dI/dU primer tolerance). FIG. 2B shows fitness_combined scores indicative of the ability of a KOD variant to tolerate dU in the template strand (by showing improved dU primer tolerance). FIG. 2C shows fitness_combined scores indicative of the ability of a KOD variant to incorporate dUTP into an extension product and to use extension products containing uracil as templates. White squares are positions excluded from analysis. Diagonal striped squares are positions with low counts, excluded due to low confidence in performance predictability.

FIG. 3 shows the correlation between KOD variants with enrichment in different conditions (i.e., dU and dI primers, dU primers, dUTP incorporation). Variants with mutations in the N-terminal domain are shown as circles; variants with mutations in the exonuclease domain are shown as diamonds

FIG. 4 shows the distribution of fitness_combined scores for combinatorial substitution KOD polymerase variants from the 7p6s library identified to have improved tolerance of templates comprising dU (by showing improved dU primer tolerance), or dU and dI (by showing improved dI/dU primer tolerance), or improved dUTP incorporation tolerance. Distributions are grouped by the number of substitutions on the x-axis; aa_edit_distance. Top plots show fitness_combined score distributions for variants with dUTP incorporation tolerance (U425T0). Middle plots show fitness_combined score distribution for variants with dU and dI primer tolerance (4xdU_3xdI). Bottom plots show fitness_combined score distributions for variants with dU primer tolerance (26xdU).

FIG. 5 shows the fitness_combined score of combinatorial substitution KOD polymerase variants from the 15p2s library identified to have improved tolerance of templates comprising dU (by showing improved dU primer tolerance), or dU and dI (by showing improved dI/dU primer tolerance), or improved dUTP incorporation tolerance. Bottom bars indicate fitness_combined scores for dU primer tolerance (26xdU) in each variant. Midddle bars indicate fitness_combined scores for dU and dI primer tolerance (4xdU_3xdI) in each variant. Top bars indicate fitness_combined scores for dUTP incorporation tolerance (U425T0) in each variant.

### DETAILED DESCRIPTION

In some aspects, this disclosure provides Thermococcus kodakarensis (KOD) polymerase variants. KOD polymerase belongs to the archaeal B-family of polymerases, which are of the domain archaea and are involved in DNA replication and repair. Such polymerases are frequently used in molecular biology, such as polymerase chain reaction (PCR), in which polynucleotides (i.e., polymers of nucleotides or nucleic acids) are amplified to generate an amount of DNA material suitable for use in a variety of downstream applications for which accurate amplification of DNA is important, such as DNA cloning, sequencing (e.g., next-generation sequencing, NGS), gene analysis, diagnostics, etc. Many archaeal B-family polymerases are derived from extremophiles and, as such, exhibit high thermostability, making them useful for applications that require high temperatures or large temperature changes (such as PCR, which can include steps ranging from around 50 °C to 98 °C). However, like many DNA polymerases, archaeal B-family polymerases, including KOD polymerase, are impaired in their ability to process polynucleotides that contain uracil or inosine (or derivatives thereof). Uracil and inosine are not typical DNA residues, instead usually found in RNA, but some molecular biology applications involve amplification of DNA containing uracil and inosine or amplification of DNA involving incorporation of uracil. Impaired processing of uracil or inosine (or derivatives thereof) by DNA polymerases results in inefficient amplification.

Provided herein are KOD polymerase variants comprising increased uracil template tolerance, inosine template tolerance, and/or uracil incorporation tolerance. In some embodiments, the KOD polymerase variants have increased uracil template tolerance. In some embodiments, the KOD polymerase variants have increased inosine template tolerance. For example, some KOD polymerase variants described herein amplify DNA comprising uracil and/or inosine with greater efficiency than wildtype KOD polymerase. In some embodiments, the KOD polymerase variants have increased uracil incorporation tolerance. In another example, some KOD polymerase variants described herein incorporate uracil into a synthesized strand and are able to use the synthesized strand as a template strand with greater efficiency than wildtype KOD polymerase.

### DEFINITIONS

A “Thermococcus kodakarensis (KOD) polymerase” refers to a B-family DNA polymerase of Thermococcus kodakarensis (e.g., a wildtype B-family KOD polymerase). In some embodiments, a KOD polymerase comprises an amino acid sequence having at least 90% (e.g., at least 95%, at least 98%, or at least 99% identity) to SEQ ID NO: 1. In some embodiments, a KOD polymerase comprises an amino acid sequence of SEQ ID NO: 1. In some embodiments, a KOD polymerase comprises an N-terminal domain. In some embodiments, a KOD polymerase comprises an exonuclease domain.

A “Thermococcus kodakarensis (KOD) polymerase variant” refers to a KOD polymerase that comprises at least one amino acid mutation relative to a KOD polymerase (e.g., relative to a wildtype KOD polymerase (e.g., SEQ ID NO: 1). In some embodiments, a KOD polymerase variant comprises a substitution mutation relative to a KOD polymerase. In some embodiments, a KOD polymerase variant comprises one or more substitution mutations relative to a KOD polymerase. In some embodiments, a KOD polymerase variant comprises an insertion and/or deletion mutation relative to a KOD polymerase. In some embodiments, a KOD polymerase variant comprises a substitution mutation that increases salt tolerance of the KOD polymerase variant relative to a KOD polymerase (e.g., increased activity of the KOD polymerase variant in the presence of salt and/or salt concentration relative to a KOD polymerase). In some embodiments, a KOD polymerase variant comprises a substitution mutation that increases dU template tolerance, dI template tolerance, and/or dUTP incorporation tolerance of the KOD polymerase variant. In some embodiments, a KOD polymerase variant comprises an amino acid sequence having at least 90% identity to any one of SEQ ID NOs: 2-3419. In some embodiments, a KOD polymerase variant comprises an amino acid sequence having at least 95% identity to any one of SEQ ID NOs: 2-3419. In some embodiments, a KOD polymerase variant comprises an amino acid sequence having at least 98% identity to any one of SEQ ID NOs: 2-3419. In some embodiments, a KOD polymerase variant comprises an amino acid sequence having at least 99% identity to any one of SEQ ID NOs: 2-3419. In some embodiments, a KOD polymerase variant comprises an amino acid sequence having at least 99.5% identity to any one of SEQ ID NOs: 2-3419. In some embodiments, a KOD polymerase variant comprises an amino acid sequence of any one of SEQ ID NOs: 2-3419.

In some embodiments, a KOD polymerase variant further comprising a protein tag. In some embodiments, the protein tag is a purification tag (e.g., a His tag (HHHHHH), a Myc tag, or a Flag tag). In some embodiments, a KOD polymerase variant comprising a linker (e.g., KL) between the KOD polymerase variant and the protein tag. In some embodiments, the protein tag is a secretion tag. In some embodiments, the disclosure provides a fusion protein comprising a KOD polymerase variant and a Sso7d protein. In some embodiments, the fusion protein comprising from N-terminal to C-terminal the KOD polymerase variant and the Sso7d protein. Sso7d is a nonspecific DNA-binding protein from the crenarchaeon Sulfolobus solfataricus or a variant thereof (e.g. a protein comprising at least 95% identity to wildtype Sso7d from Sulfolobus solfataricus or a homolog of Sso7d). In some embodiments, the KOD polymerase variants described herein comprise a Sso7d domain. In some embodiments, Sso7d comprises an amino acid sequence of SEQ ID NO: 3419

((M)ATVKFKYKGEEKEVDISKIKKVWRVGKMISFTYDEGGGKTGR
GAVSEKDAPKELLQMLEKQKK, SEQ ID NO: 3419)

“Uracil template tolerance” or “dU template tolerance” refers to an ability of a DNA polymerase, a KOD polymerase, or a KOD polymerase variant to amplify a DNA template containing uracil or a uracil derivative. In some embodiments (e.g., in the Examples section), uracil template tolerance refers to an ability to amplify a DNA template containing uracil, where the DNA template is generated by extending a primer bound to a first DNA strand, wherein the primer comprises uracil or a uracil derivative. This type of uracil template tolerance is referred to herein as “dU primer tolerance”. In another embodiment, a DNA template containing uracil is generated by incorporation of dUTP throughout a synthesized strand during amplification. This type of uracil template tolerance is referred to herein as “dUTP incorporation tolerance”. Wildtype KOD polymerase is known to have relatively low uracil template tolerance compared to KOD polymerase variants described herein. A polymerase with increased uracil template tolerance indicates an increased activity of the polymerase to amplify DNA that comprises uracil or a uracil derivative relative to a control polymerase (e.g., a wildtype KOD polymerase) (e.g., an increase of at least 0.5 fold, at least 1 fold, at least 1.5 fold, at least 2 fold, at least 5 fold, or at least 10 fold). In some embodiments, a polymerase (e.g., a KOD polymerase variant) with increased uracil template tolerance has 0.5-10 fold, 0.5-5 fold, or 0.5-3 fold increase in activity for a template DNA comprising uracil compared to a wildtype polymerase (e.g., wildtype KOD polymerase).

“Inosine template tolerance” or “dI template tolerance” refers to an ability of a DNA polymerase, a KOD polymerase, or a KOD polymerase variant to amplify a DNA template containing inosine or an inosine derivative. In some embodiments, inosine template tolerance refers to an ability to amplify a DNA template containing inosine, where the DNA template is generated by extending a primer bound to a first DNA strand, wherein the primer comprises inosine or an inosine derivative. This type of inosine template tolerance is referred to herein as “dI primer tolerance”. In another embodiment, a DNA template containing inosine is generated by incorporation of dITP throughout a synthesized strand during amplification. This type of inosine template tolerance is referred to herein as “dITP incorporation tolerance”. Wildtype KOD polymerase is known to have relatively low inosine template tolerance compared to KOD polymerase variants described herein. A polymerase with increased Inosine template tolerance indicates an increased activity of the polymerase to amplify DNA that comprises inosine or a inosine derivative relative to a control polymerase (e.g., a wildtype KOD polymerase) (e.g., an increase of at least 0.5 fold, at least 1 fold, at least 1.5 fold, at least 2 fold, at least 5 fold, or at least 10 fold). In some embodiments, a polymerase (e.g., a KOD polymerase variant) with increased inosine template tolerance has 0.5-10 fold, 0.5-5 fold, or 0.5-3 fold increase in activity for a template DNA comprising inosine compared to a wildtype polymerase (e.g., wildtype KOD polymerase).

In some aspects, the present disclosure provides polymerase variants with “dU/dI template tolerance”, which refers to an ability of a polymerase or polymerase variant to amplify a DNA template containing uracil and inosine (or derivatives thereof). In some embodiments, dU/dI template tolerance refers to an ability to amplify a DNA template containing both uracil and inosine, wherein the DNA template containing uracil and inosine is generated by extending a primer bound to a first DNA strand, wherein the primer comprises both uracil and inosine, or derivatives thereof. This type of dU/dI template tolerance is referred to herein as “dU/dI primer tolerance”.

“Uracil incorporation tolerance” or “dUTP incorporation tolerance” refers to an ability of a DNA polymerase, a KOD polymerase, or a KOD polymerase variant to first incorporate uracil (dUTP) into a first DNA strand complementary to a template, and second, to synthesize a second strand that is complementary to the uracil (dUTP)-containing first DNA strand. Wildtype KOD polymerase is known to have relatively low uracil incorporation tolerance in amplification processes such as PCR compared to KOD polymerase variants described herein. A polymerase with increased dUTP incorporation tolerance indicates an increased ability of the polymerase to amplify DNA in the presence of dUTP, relative to a control polymerase (e.g., a wildtype KOD polymerase). Without being bound to theory, dUTP incorporation tolerance is thought to be primarily a function of the polymerase's ability to amplify to synthesize a second strand that is complementary to the uracil (dUTP)-containing first DNA strand made by incorporating the dUTP into the first DNA strand, and less so a function of the polymerases ability to incorporate dUTP into the first synthesized strand (e.g., an increase of at least 0.5 fold, at least 1 fold, at least 1.5 fold, at least 2 fold, at least 5 fold, or at least 10 fold). In some embodiments, a polymerase (e.g., a KOD polymerase variant) with increased dUTP tolerance has 0.5-10 fold, 0.5-5 fold, or 0.5-3 fold increase in activity for a template DNA comprising dUTP compared to a wildtype polymerase (e.g., wildtype KOD polymerase).

In some embodiments, a KOD polymerase variant comprises 1, 2, 3, 4, 5, 6, 7, 8, 9, 10, 11, 12, 13, 14, or 15 amino acid substitutions at positions corresponding to amino acid positions of SEQ ID NO: 1.

In some embodiments, this disclosure provides a KOD polymerase variant comprising a mutation (e.g., a substitution, an insertion, or a deletion) at a position corresponding to a reference (e.g., wild-type) KOD polymerase. A KOD polymerase variant having a “position corresponding to” a reference amino acid sequence (e.g., a reference KOD polymerase amino acid sequence like SEQ ID NO: 1) refers to a particular amino acid in the KOD polymerase variant relative to the reference KOD polymerase.

A KOD polymerase variant may be identified relative to a reference sequence. In other words, the reference sequence can be used as a standard for mapping mutations between different KOD polymerase variants. This mapping may be used to compare KOD DNA polymerase variants comprising insertions or deletions that shift the location of a mutation relative to the reference sequence. In some embodiments, determining a position of a KOD polymerase variant corresponding to a reference sequence comprises aligning the KOD polymerase sequence and the KOD polymerase variant sequence (e.g., using Clustal Omega as described in Madeira et al., Nucleic Acids Res. 2024 July; 52(W1) W521-W525. PMID: 38597606). In some embodiments, this is determined using a structural alignment (e.g., as described in Bittrich et al., Bioinformatics, Volume 40, Issue 6, June 2024, doi.org/10.1093/bioinformatics/btae370). Directly below are four examples all relating to a KOD polymerase variant comprising a methionine at a position corresponding to Y38 of a reference sequence. All the below KOD polymerase variants in these four examples would fall within the scope of, for example, a KOD polymerase variant comprising a methionine at a position corresponding to Y38 of a reference sequence (Ref).

In a first example, the reference sequence (Ref) and the target sequence (Tar) align at every amino acid between positions 4 and 37 of the reference sequence. Position 38 of the target sequence corresponds to position 38 of the reference sequence. Position 38 of the target sequence is methionine. Position 38 of the reference sequence is tyrosine.

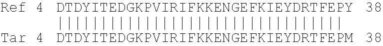

In a second example, the target sequence has a three amino acid deletion and a tyrosine to methionine substitution relative to the reference sequence. Because of the deletion, position 35 of the target sequence corresponds to position 38 of the reference sequence. Position 35 of the target sequence is methionine. Position 38 of the reference sequence is tyrosine.

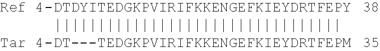

In a third example, the target sequence has a four amino acid insertion and a tyrosine to methionine substitution relative to the reference sequence. Because of the insertion, position 42 of the target sequence corresponds to position 38 of the reference sequence. Position 42 of the target sequence is methionine. Position 38 of the reference sequence is tyrosine.

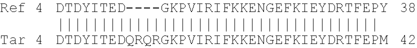

In a fourth example, the target sequence differs in sequence at amino position 38, 42, 43, and 44. Position 38 of the target sequence corresponds to position 38 of the reference sequence. Position 38 of the target sequence is methionine. Position 38 of the reference sequence is tyrosine.

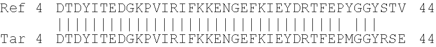

In some instances, two different protein sequences may not correspond with one another. For example, this may occur when the sequence identity or structural identity between two protein sequences is so low that an alignment cannot be made.

Some embodiments relate to amino acid sequences or nucleotide sequences having some percentage identity to a reference amino acid sequence or reference nucleotide sequence, respectively. As used herein, the term “identity” refers to the degree to which two or more sequences (e.g., amino acid sequences, nucleotide sequences) are related, as determined by the number of matches between each sequence. “Percent (%) identity” refers to the percentage of residues or nucleotides in a first amino acid or first nucleic acid sequence that are identical to the residues in a second amino acid sequence or second nucleic acid sequence. The skilled artisan will understand that determining percent identity may require a step of aligning a first and second sequence and/or introducing gaps. Several algorithms for aligning sequences are known in the art, for example, on the worldwide web at blast.ncbi.nlm.nih.gov/Blast.cgi and genome.jp/tools-bin/clustalw.

### KOD Polymerase Variants

### Single Assay Variants

### I2

In some embodiments, a KOD polymerase variant comprises: a D, E, F, H, K, L, N, T, V, or W at a position corresponding to I2 of SEQ ID NO: 1. In some embodiments, a KOD polymerase variant comprises: a D, E, F, H, K, L, N, T, V, or W at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, H, K, L, N, T, V, or W at position I2 of SEQ ID NO: 1.

In some embodiments, this disclosure provides a KOD polymerase variant comprising: a D at a position corresponding to I2 of SEQ ID NO: 1. In some embodiments, this disclosure provides a KOD polymerase variant comprising: a D at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position I2 of SEQ ID NO: 1.

In some embodiments, this disclosure provides a KOD polymerase variant comprising: an E at a position corresponding to I2 of SEQ ID NO: 1. In some embodiments, this disclosure provides a KOD polymerase variant comprising: an E at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position I2 of SEQ ID NO: 1.

In some embodiments, this disclosure provides a KOD polymerase variant comprising: a F at a position corresponding to I2 of SEQ ID NO: 1. In some embodiments, this disclosure provides a KOD polymerase variant comprising: a F at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I2 of SEQ ID NO: 1.

In some embodiments, this disclosure provides a KOD polymerase variant comprising: a H at a position corresponding to I2 of SEQ ID NO: 1. In some embodiments, this disclosure provides a KOD polymerase variant comprising: a H at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I2 of SEQ ID NO: 1.

In some embodiments, this disclosure provides a KOD polymerase variant comprising: a K at a position corresponding to I2 of SEQ ID NO: 1. In some embodiments, this disclosure provides a KOD polymerase variant comprising: a K at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I2 of SEQ ID NO: 1.

In some embodiments, this disclosure provides a KOD polymerase variant comprising: a L at a position corresponding to I2 of SEQ ID NO: 1. In some embodiments, this disclosure provides a KOD polymerase variant comprising: a L at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position I2 of SEQ ID NO: 1.

In some embodiments, this disclosure provides a KOD polymerase variant comprising: a N at a position corresponding to I2 of SEQ ID NO: 1. In some embodiments, this disclosure provides a KOD polymerase variant comprising: a N at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position I2 of SEQ ID NO: 1.

In some embodiments, this disclosure provides a KOD polymerase variant comprising: a T at a position corresponding to I2 of SEQ ID NO: 1. In some embodiments, this disclosure provides a KOD polymerase variant comprising: a T at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position I2 of SEQ ID NO: 1.

In some embodiments, this disclosure provides a KOD polymerase variant comprising: a V at a position corresponding to I2 of SEQ ID NO: 1. In some embodiments, this disclosure provides a KOD polymerase variant comprising: a V at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position I2 of SEQ ID NO: 1.

In some embodiments, this disclosure provides a KOD polymerase variant comprising: a W at a position corresponding to I2 of SEQ ID NO: 1. In some embodiments, this disclosure provides a KOD polymerase variant comprising: a W at position I2 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I2 of SEQ ID NO: 1.

### L3

In some embodiments, a KOD polymerase variant comprises: a D, H, I, S, T, or Y at a position corresponding to L3 of SEQ ID NO: 1. In some embodiments, a KOD polymerase variant comprises: a D, H, I, S, T, or Y at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, H, I, S, T, or Y at position L3 of SEQ ID NO: 1.

In some embodiments, this disclosure provides a KOD polymerase variant comprising: a D at a position corresponding to L3 of SEQ ID NO: 1. In some embodiments, this disclosure provides a KOD polymerase variant comprising: a D at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position L3 of SEQ ID NO: 1.

In some embodiments, this disclosure provides a KOD polymerase variant comprising: a H at a position corresponding to L3 of SEQ ID NO: 1. In some embodiments, this disclosure provides a KOD polymerase variant comprising: a H at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position L3 of SEQ ID NO: 1.

In some embodiments, this disclosure provides a KOD polymerase variant comprising: an I at a position corresponding to L3 of SEQ ID NO: 1. In some embodiments, this disclosure provides a KOD polymerase variant comprising: an I at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position L3 of SEQ ID NO: 1.

In some embodiments, this disclosure provides a KOD polymerase variant comprising: a S at a position corresponding to L3 of SEQ ID NO: 1. In some embodiments, this disclosure provides a KOD polymerase variant comprising: a S at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position L3 of SEQ ID NO: 1.

In some embodiments, this disclosure provides a KOD polymerase variant comprising: a T at a position corresponding to L3 of SEQ ID NO: 1. In some embodiments, this disclosure provides a KOD polymerase variant comprising: a T at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position L3 of SEQ ID NO: 1.

In some embodiments, this disclosure provides a KOD polymerase variant comprising: a Y at a position corresponding to L3 of SEQ ID NO: 1. In some embodiments, this disclosure provides a KOD polymerase variant comprising: a Y at position L3 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position L3 of SEQ ID NO: 1.

### D4

In some embodiments, a KOD polymerase variant comprises: an I, L, M, P, or Y at a position corresponding to D4 of SEQ ID NO: 1. In some embodiments, a KOD polymerase variant comprises: an I, L, M, P, or Y at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I, L, M, P, or Y at position D4 of SEQ ID NO: 1.

In some embodiments, this disclosure provides a KOD polymerase variant comprising: an I at a position corresponding to D4 of SEQ ID NO: 1. In some embodiments, this disclosure provides a KOD polymerase variant comprising: an I at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D4 of SEQ ID NO: 1.

In some embodiments, this disclosure provides a KOD polymerase variant comprising: a L at a position corresponding to D4 of SEQ ID NO: 1. In some embodiments, this disclosure provides a KOD polymerase variant comprising: a L at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D4 of SEQ ID NO: 1.

In some embodiments, this disclosure provides a KOD polymerase variant comprising: a M at a position corresponding to D4 of SEQ ID NO: 1. In some embodiments, this disclosure provides a KOD polymerase variant comprising: a M at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D4 of SEQ ID NO: 1.

In some embodiments, this disclosure provides a KOD polymerase variant comprising: a P at a position corresponding to D4 of SEQ ID NO: 1. In some embodiments, this disclosure provides a KOD polymerase variant comprising: a P at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D4 of SEQ ID NO: 1.

In some embodiments, this disclosure provides a KOD polymerase variant comprising: a Y at a position corresponding to D4 of SEQ ID NO: 1. In some embodiments, this disclosure provides a KOD polymerase variant comprising: a Y at position D4 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D4 of SEQ ID NO: 1.

### T5

In some embodiments, a KOD polymerase variant comprises: a D, E, F, G, H, L, W, or Y at a position corresponding to T5 of SEQ ID NO: 1. In some embodiments, a KOD polymerase variant comprises: a D, E, F, G, H, L, W, or Y at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, G, H, L, W, or Y at position T5 of SEQ ID NO: 1.

In some embodiments, this disclosure provides a KOD polymerase variant comprising: a D at a position corresponding to T5 of SEQ ID NO: 1. In some embodiments, this disclosure provides a KOD polymerase variant comprising: a D at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position T5 of SEQ ID NO: 1.

In some embodiments, this disclosure provides a KOD polymerase variant comprising: an E at a position corresponding to T5 of SEQ ID NO: 1. In some embodiments, this disclosure provides a KOD polymerase variant comprising: an E at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position T5 of SEQ ID NO: 1.

In some embodiments, this disclosure provides a KOD polymerase variant comprising: a F at a position corresponding to T5 of SEQ ID NO: 1. In some embodiments, this disclosure provides a KOD polymerase variant comprising: a F at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position T5 of SEQ ID NO: 1.

In some embodiments, this disclosure provides a KOD polymerase variant comprising: a G at a position corresponding to T5 of SEQ ID NO: 1. In some embodiments, this disclosure provides a KOD polymerase variant comprising: a G at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position T5 of SEQ ID NO: 1.

In some embodiments, this disclosure provides a KOD polymerase variant comprising: a H at a position corresponding to T5 of SEQ ID NO: 1. In some embodiments, this disclosure provides a KOD polymerase variant comprising: a H at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position T5 of SEQ ID NO: 1.

In some embodiments, this disclosure provides a KOD polymerase variant comprising: a L at a position corresponding to T5 of SEQ ID NO: 1. In some embodiments, this disclosure provides a KOD polymerase variant comprising: a L at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position T5 of SEQ ID NO: 1.

In some embodiments, this disclosure provides a KOD polymerase variant comprising: a W at a position corresponding to T5 of SEQ ID NO: 1. In some embodiments, this disclosure provides a KOD polymerase variant comprising: a W at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position T5 of SEQ ID NO: 1.

In some embodiments, this disclosure provides a KOD polymerase variant comprising: a Y at a position corresponding to T5 of SEQ ID NO: 1. In some embodiments, this disclosure provides a KOD polymerase variant comprising: a Y at position T5 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position T5 of SEQ ID NO: 1.

### D6

In some embodiments, this disclosure provides a KOD polymerase variant comprising: a L at a position corresponding to D6 of SEQ ID NO: 1. In some embodiments, this disclosure provides a KOD polymerase variant comprising: a L at position D6 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D6 of SEQ ID NO: 1.

### Y7

In some embodiments, a KOD polymerase variant comprises: an A, C, D, E, F, G, H, I, K, L, M, N, P, Q, R, S, T, V, or W at a position corresponding to Y7 of SEQ ID NO: 1. In some embodiments, a KOD polymerase variant comprises: an A, C, D, E, F, G, H, I, K, L, M, N, P, Q, R, S, T, V, or W at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, G, H, I, K, L, M, N, P, Q, R, S, T, V, or W at position Y7 of SEQ ID NO: 1.

In some embodiments, this disclosure provides a KOD polymerase variant comprising: an A at a position corresponding to Y7 of SEQ ID NO: 1. In some embodiments, this disclosure provides a KOD polymerase variant comprising: an A at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position Y7 of SEQ ID NO: 1.

In some embodiments, this disclosure provides a KOD polymerase variant comprising: a C at a position corresponding to Y7 of SEQ ID NO: 1. In some embodiments, this disclosure provides a KOD polymerase variant comprising: a C at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position Y7 of SEQ ID NO: 1.

In some embodiments, this disclosure provides a KOD polymerase variant comprising: a D at a position corresponding to Y7 of SEQ ID NO: 1. In some embodiments, this disclosure provides a KOD polymerase variant comprising: a D at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y7 of SEQ ID NO: 1.

In some embodiments, this disclosure provides a KOD polymerase variant comprising: an E at a position corresponding to Y7 of SEQ ID NO: 1. In some embodiments, this disclosure provides a KOD polymerase variant comprising: an E at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y7 of SEQ ID NO: 1.

In some embodiments, this disclosure provides a KOD polymerase variant comprising: a F at a position corresponding to Y7 of SEQ ID NO: 1. In some embodiments, this disclosure provides a KOD polymerase variant comprising: a F at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position Y7 of SEQ ID NO: 1.

In some embodiments, this disclosure provides a KOD polymerase variant comprising: a G at a position corresponding to Y7 of SEQ ID NO: 1. In some embodiments, this disclosure provides a KOD polymerase variant comprising: a G at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position Y7 of SEQ ID NO: 1.

In some embodiments, this disclosure provides a KOD polymerase variant comprising: a H at a position corresponding to Y7 of SEQ ID NO: 1. In some embodiments, this disclosure provides a KOD polymerase variant comprising: a H at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position Y7 of SEQ ID NO: 1.

In some embodiments, this disclosure provides a KOD polymerase variant comprising: an I at a position corresponding to Y7 of SEQ ID NO: 1. In some embodiments, this disclosure provides a KOD polymerase variant comprising: an I at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position Y7 of SEQ ID NO: 1.

In some embodiments, this disclosure provides a KOD polymerase variant comprising: a K at a position corresponding to Y7 of SEQ ID NO: 1. In some embodiments, this disclosure provides a KOD polymerase variant comprising: a K at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position Y7 of SEQ ID NO: 1.

In some embodiments, this disclosure provides a KOD polymerase variant comprising: a L at a position corresponding to Y7 of SEQ ID NO: 1. In some embodiments, this disclosure provides a KOD polymerase variant comprising: a L at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position Y7 of SEQ ID NO: 1.

In some embodiments, this disclosure provides a KOD polymerase variant comprising: a M at a position corresponding to Y7 of SEQ ID NO: 1. In some embodiments, this disclosure provides a KOD polymerase variant comprising: a M at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position Y7 of SEQ ID NO: 1.

In some embodiments, this disclosure provides a KOD polymerase variant comprising: a N at a position corresponding to Y7 of SEQ ID NO: 1. In some embodiments, this disclosure provides a KOD polymerase variant comprising: a N at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position Y7 of SEQ ID NO: 1.

In some embodiments, this disclosure provides a KOD polymerase variant comprising: a P at a position corresponding to Y7 of SEQ ID NO: 1. In some embodiments, this disclosure provides a KOD polymerase variant comprising: a P at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y7 of SEQ ID NO: 1.

In some embodiments, this disclosure provides a KOD polymerase variant comprising: a Q at a position corresponding to Y7 of SEQ ID NO: 1. In some embodiments, this disclosure provides a KOD polymerase variant comprising: a Q at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position Y7 of SEQ ID NO: 1.

In some embodiments, this disclosure provides a KOD polymerase variant comprising: a R at a position corresponding to Y7 of SEQ ID NO: 1. In some embodiments, this disclosure provides a KOD polymerase variant comprising: a R at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position Y7 of SEQ ID NO: 1.

In some embodiments, this disclosure provides a KOD polymerase variant comprising: a S at a position corresponding to Y7 of SEQ ID NO: 1. In some embodiments, this disclosure provides a KOD polymerase variant comprising: a S at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position Y7 of SEQ ID NO: 1.

In some embodiments, this disclosure provides a KOD polymerase variant comprising: a T at a position corresponding to Y7 of SEQ ID NO: 1. In some embodiments, this disclosure provides a KOD polymerase variant comprising: a T at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position Y7 of SEQ ID NO: 1.

In some embodiments, this disclosure provides a KOD polymerase variant comprising: a V at a position corresponding to Y7 of SEQ ID NO: 1. In some embodiments, this disclosure provides a KOD polymerase variant comprising: a V at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position Y7 of SEQ ID NO: 1.

In some embodiments, this disclosure provides a KOD polymerase variant comprising: a W at a position corresponding to Y7 of SEQ ID NO: 1. In some embodiments, this disclosure provides a KOD polymerase variant comprising: a W at position Y7 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position Y7 of SEQ ID NO: 1.

### I8

In some embodiments, a KOD polymerase variant comprises: a D, E, H, or T at a position corresponding to I8 of SEQ ID NO: 1. In some embodiments, a KOD polymerase variant comprises: a D, E, H, or T at position I8 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, H, or T at position I8 of SEQ ID NO: 1.

In some embodiments, this disclosure provides a KOD polymerase variant comprising: a D at a position corresponding to I8 of SEQ ID NO: 1. In some embodiments, this disclosure provides a KOD polymerase variant comprising: a D at position I8 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position I8 of SEQ ID NO: 1.

In some embodiments, this disclosure provides a KOD polymerase variant comprising: an E at a position corresponding to I8 of SEQ ID NO: 1. In some embodiments, this disclosure provides a KOD polymerase variant comprising: an E at position I8 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position I8 of SEQ ID NO: 1.

In some embodiments, this disclosure provides a KOD polymerase variant comprising: a H at a position corresponding to I8 of SEQ ID NO: 1. In some embodiments, this disclosure provides a KOD polymerase variant comprising: a H at position I8 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I8 of SEQ ID NO: 1.

In some embodiments, this disclosure provides a KOD polymerase variant comprising: a T at a position corresponding to I8 of SEQ ID NO: 1. In some embodiments, this disclosure provides a KOD polymerase variant comprising: a T at position I8 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position I8 of SEQ ID NO: 1.

### T9

In some embodiments, a KOD polymerase variant comprises: an A, C, D, E, F, H, I, L, N, R, or Y at a position corresponding to T9 of SEQ ID NO: 1. In some embodiments, a KOD polymerase variant comprises: an A, C, D, E, F, H, I, L, N, R, or Y at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, H, I, L, N, R, or Y at position T9 of SEQ ID NO: 1.

In some embodiments, this disclosure provides a KOD polymerase variant comprising: an A at a position corresponding to T9 of SEQ ID NO: 1. In some embodiments, this disclosure provides a KOD polymerase variant comprising: an A at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position T9 of SEQ ID NO: 1.

In some embodiments, this disclosure provides a KOD polymerase variant comprising: a C at a position corresponding to T9 of SEQ ID NO: 1. In some embodiments, this disclosure provides a KOD polymerase variant comprising: a C at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position T9 of SEQ ID NO: 1.

In some embodiments, this disclosure provides a KOD polymerase variant comprising: a D at a position corresponding to T9 of SEQ ID NO: 1. In some embodiments, this disclosure provides a KOD polymerase variant comprising: a D at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position T9 of SEQ ID NO: 1.

In some embodiments, this disclosure provides a KOD polymerase variant comprising: an E at a position corresponding to T9 of SEQ ID NO: 1. In some embodiments, this disclosure provides a KOD polymerase variant comprising: an E at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position T9 of SEQ ID NO: 1.

In some embodiments, this disclosure provides a KOD polymerase variant comprising: a F at a position corresponding to T9 of SEQ ID NO: 1. In some embodiments, this disclosure provides a KOD polymerase variant comprising: a F at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position T9 of SEQ ID NO: 1.

In some embodiments, this disclosure provides a KOD polymerase variant comprising: a H at a position corresponding to T9 of SEQ ID NO: 1. In some embodiments, this disclosure provides a KOD polymerase variant comprising: a H at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position T9 of SEQ ID NO: 1.

In some embodiments, this disclosure provides a KOD polymerase variant comprising: an I at a position corresponding to T9 of SEQ ID NO: 1. In some embodiments, this disclosure provides a KOD polymerase variant comprising: an I at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position T9 of SEQ ID NO: 1.

In some embodiments, this disclosure provides a KOD polymerase variant comprising: a L at a position corresponding to T9 of SEQ ID NO: 1. In some embodiments, this disclosure provides a KOD polymerase variant comprising: a L at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position T9 of SEQ ID NO: 1.

In some embodiments, this disclosure provides a KOD polymerase variant comprising: a N at a position corresponding to T9 of SEQ ID NO: 1. In some embodiments, this disclosure provides a KOD polymerase variant comprising: a N at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position T9 of SEQ ID NO: 1.

In some embodiments, this disclosure provides a KOD polymerase variant comprising: a R at a position corresponding to T9 of SEQ ID NO: 1. In some embodiments, this disclosure provides a KOD polymerase variant comprising: a R at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position T9 of SEQ ID NO: 1.

In some embodiments, this disclosure provides a KOD polymerase variant comprising: a Y at a position corresponding to T9 of SEQ ID NO: 1. In some embodiments, this disclosure provides a KOD polymerase variant comprising: a Y at position T9 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position T9 of SEQ ID NO: 1.

### E10

In some embodiments, a KOD polymerase variant comprises: a P or Q at a position corresponding to E10 of SEQ ID NO: 1. In some embodiments, a KOD polymerase variant comprises: a P or Q at position E10 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P or Q at position E10 of SEQ ID NO: 1.

In some embodiments, this disclosure provides a KOD polymerase variant comprising: a P at a position corresponding to E10 of SEQ ID NO: 1. In some embodiments, this disclosure provides a KOD polymerase variant comprising: a P at position E10 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E10 of SEQ ID NO: 1.

In some embodiments, this disclosure provides a KOD polymerase variant comprising: a Q at a position corresponding to E10 of SEQ ID NO: 1. In some embodiments, this disclosure provides a KOD polymerase variant comprising: a Q at position E10 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position E10 of SEQ ID NO: 1.

### D11

In some embodiments, a KOD polymerase variant comprises: a Q or T at a position corresponding to D11 of SEQ ID NO: 1. In some embodiments, a KOD polymerase variant comprises: a Q or T at position D11 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q or T at position D11 of SEQ ID NO: 1.

In some embodiments, this disclosure provides a KOD polymerase variant comprising: a Q at a position corresponding to D11 of SEQ ID NO: 1. In some embodiments, this disclosure provides a KOD polymerase variant comprising: a Q at position D11 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D11 of SEQ ID NO: 1.

In some embodiments, this disclosure provides a KOD polymerase variant comprising: a T at a position corresponding to D11 of SEQ ID NO: 1. In some embodiments, this disclosure provides a KOD polymerase variant comprising: a T at position D11 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D11 of SEQ ID NO: 1.

### G12

In some embodiments, a KOD polymerase variant comprises: a C, Q, T, or V at a position corresponding to G12 of SEQ ID NO: 1. In some embodiments, a KOD polymerase variant comprises: a C, Q, T, or V at position G12 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, Q, T, or V at position G12 of SEQ ID NO: 1.

In some embodiments, this disclosure provides a KOD polymerase variant comprising: a C at a position corresponding to G12 of SEQ ID NO: 1. In some embodiments, this disclosure provides a KOD polymerase variant comprising: a C at position G12 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position G12 of SEQ ID NO: 1.

In some embodiments, this disclosure provides a KOD polymerase variant comprising: a Q at a position corresponding to G12 of SEQ ID NO: 1. In some embodiments, this disclosure provides a KOD polymerase variant comprising: a Q at position G12 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position G12 of SEQ ID NO: 1.

In some embodiments, this disclosure provides a KOD polymerase variant comprising: a T at a position corresponding to G12 of SEQ ID NO: 1. In some embodiments, this disclosure provides a KOD polymerase variant comprising: a T at position G12 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position G12 of SEQ ID NO: 1.

In some embodiments, this disclosure provides a KOD polymerase variant comprising: a V at a position corresponding to G12 of SEQ ID NO: 1. In some embodiments, this disclosure provides a KOD polymerase variant comprising: a V at position G12 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position G12 of SEQ ID NO: 1.

### K13

In some embodiments, a KOD polymerase variant comprises: a G, H, T, V, or W at a position corresponding to K13 of SEQ ID NO: 1. In some embodiments, a KOD polymerase variant comprises: a G, H, T, V, or W at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G, H, T, V, or W at position K13 of SEQ ID NO: 1.

In some embodiments, this disclosure provides a KOD polymerase variant comprising: a G at a position corresponding to K13 of SEQ ID NO: 1. In some embodiments, this disclosure provides a KOD polymerase variant comprising: a G at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position K13 of SEQ ID NO: 1.

In some embodiments, this disclosure provides a KOD polymerase variant comprising: a H at a position corresponding to K13 of SEQ ID NO: 1. In some embodiments, this disclosure provides a KOD polymerase variant comprising: a H at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position K13 of SEQ ID NO: 1.

In some embodiments, this disclosure provides a KOD polymerase variant comprising: a T at a position corresponding to K13 of SEQ ID NO: 1. In some embodiments, this disclosure provides a KOD polymerase variant comprising: a T at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position K13 of SEQ ID NO: 1.

In some embodiments, this disclosure provides a KOD polymerase variant comprising: a V at a position corresponding to K13 of SEQ ID NO: 1. In some embodiments, this disclosure provides a KOD polymerase variant comprising: a V at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position K13 of SEQ ID NO: 1.

In some embodiments, this disclosure provides a KOD polymerase variant comprising: a W at a position corresponding to K13 of SEQ ID NO: 1. In some embodiments, this disclosure provides a KOD polymerase variant comprising: a W at position K13 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position K13 of SEQ ID NO: 1.

### P14

In some embodiments, a KOD polymerase variant comprises: a C, D, E, F, H, M, Q, S, T, or W at a position corresponding to P14 of SEQ ID NO: 1. In some embodiments, a KOD polymerase variant comprises: a C, D, E, F, H, M, Q, S, T, or W at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, F, H, M, Q, S, T, or W at position P14 of SEQ ID NO: 1.

In some embodiments, this disclosure provides a KOD polymerase variant comprising: a C at a position corresponding to P14 of SEQ ID NO: 1. In some embodiments, this disclosure provides a KOD polymerase variant comprising: a C at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position P14 of SEQ ID NO: 1.

In some embodiments, this disclosure provides a KOD polymerase variant comprising: a D at a position corresponding to P14 of SEQ ID NO: 1. In some embodiments, this disclosure provides a KOD polymerase variant comprising: a D at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position P14 of SEQ ID NO: 1.

In some embodiments, this disclosure provides a KOD polymerase variant comprising: an E at a position corresponding to P14 of SEQ ID NO: 1. In some embodiments, this disclosure provides a KOD polymerase variant comprising: an E at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position P14 of SEQ ID NO: 1.

In some embodiments, this disclosure provides a KOD polymerase variant comprising: a F at a position corresponding to P14 of SEQ ID NO: 1. In some embodiments, this disclosure provides a KOD polymerase variant comprising: a F at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position P14 of SEQ ID NO: 1.

In some embodiments, this disclosure provides a KOD polymerase variant comprising: a H at a position corresponding to P14 of SEQ ID NO: 1. In some embodiments, this disclosure provides a KOD polymerase variant comprising: a H at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position P14 of SEQ ID NO: 1.

In some embodiments, this disclosure provides a KOD polymerase variant comprising: a M at a position corresponding to P14 of SEQ ID NO: 1. In some embodiments, this disclosure provides a KOD polymerase variant comprising: a M at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position P14 of SEQ ID NO: 1.

In some embodiments, this disclosure provides a KOD polymerase variant comprising: a Q at a position corresponding to P14 of SEQ ID NO: 1. In some embodiments, this disclosure provides a KOD polymerase variant comprising: a Q at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position P14 of SEQ ID NO: 1.

In some embodiments, this disclosure provides a KOD polymerase variant comprising: a S at a position corresponding to P14 of SEQ ID NO: 1. In some embodiments, this disclosure provides a KOD polymerase variant comprising: a S at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position P14 of SEQ ID NO: 1.

In some embodiments, this disclosure provides a KOD polymerase variant comprising: a T at a position corresponding to P14 of SEQ ID NO: 1. In some embodiments, this disclosure provides a KOD polymerase variant comprising: a T at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position P14 of SEQ ID NO: 1.

In some embodiments, this disclosure provides a KOD polymerase variant comprising: a W at a position corresponding to P14 of SEQ ID NO: 1. In some embodiments, this disclosure provides a KOD polymerase variant comprising: a W at position P14 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position P14 of SEQ ID NO: 1.

### V15

In some embodiments, a KOD polymerase variant comprises: a Q or W at a position corresponding to V15 of SEQ ID NO: 1. In some embodiments, a KOD polymerase variant comprises: a Q or W at position V15 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q or W at position V15 of SEQ ID NO: 1.

In some embodiments, this disclosure provides a KOD polymerase variant comprising: a Q at a position corresponding to V15 of SEQ ID NO: 1. In some embodiments, this disclosure provides a KOD polymerase variant comprising: a Q at position V15 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position V15 of SEQ ID NO: 1.

In some embodiments, this disclosure provides a KOD polymerase variant comprising: a W at a position corresponding to V15 of SEQ ID NO: 1. In some embodiments, this disclosure provides a KOD polymerase variant comprising: a W at position V15 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position V15 of SEQ ID NO: 1.

### I16

In some embodiments, a KOD polymerase variant comprises: an A, C, F, H, K, S, W, or Y at a position corresponding to I16 of SEQ ID NO: 1. In some embodiments, a KOD polymerase variant comprises: an A, C, F, H, K, S, W, or Y at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, H, K, S, W, or Y at position I16 of SEQ ID NO: 1.

In some embodiments, this disclosure provides a KOD polymerase variant comprising: an A at a position corresponding to I16 of SEQ ID NO: 1. In some embodiments, this disclosure provides a KOD polymerase variant comprising: an A at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position I16 of SEQ ID NO: 1.

In some embodiments, this disclosure provides a KOD polymerase variant comprising: a C at a position corresponding to I16 of SEQ ID NO: 1. In some embodiments, this disclosure provides a KOD polymerase variant comprising: a C at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position I16 of SEQ ID NO: 1.

In some embodiments, this disclosure provides a KOD polymerase variant comprising: a F at a position corresponding to I16 of SEQ ID NO: 1. In some embodiments, this disclosure provides a KOD polymerase variant comprising: a F at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I16 of SEQ ID NO: 1.

In some embodiments, this disclosure provides a KOD polymerase variant comprising: a H at a position corresponding to I16 of SEQ ID NO: 1. In some embodiments, this disclosure provides a KOD polymerase variant comprising: a H at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I16 of SEQ ID NO: 1.

In some embodiments, this disclosure provides a KOD polymerase variant comprising: a K at a position corresponding to I16 of SEQ ID NO: 1. In some embodiments, this disclosure provides a KOD polymerase variant comprising: a K at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I16 of SEQ ID NO: 1.

In some embodiments, this disclosure provides a KOD polymerase variant comprising: a S at a position corresponding to I16 of SEQ ID NO: 1. In some embodiments, this disclosure provides a KOD polymerase variant comprising: a S at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position I16 of SEQ ID NO: 1.

In some embodiments, this disclosure provides a KOD polymerase variant comprising: a W at a position corresponding to I16 of SEQ ID NO: 1. In some embodiments, this disclosure provides a KOD polymerase variant comprising: a W at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I16 of SEQ ID NO: 1.

In some embodiments, this disclosure provides a KOD polymerase variant comprising: a Y at a position corresponding to I16 of SEQ ID NO: 1. In some embodiments, this disclosure provides a KOD polymerase variant comprising: a Y at position I16 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I16 of SEQ ID NO: 1.

### R17

In some embodiments, this disclosure provides a KOD polymerase variant comprising: a P at a position corresponding to R17 of SEQ ID NO: 1. In some embodiments, this disclosure provides a KOD polymerase variant comprising: a P at position R17 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position R17 of SEQ ID NO: 1.

### I18

In some embodiments, this disclosure provides a KOD polymerase variant comprising: a Y at a position corresponding to I18 of SEQ ID NO: 1. In some embodiments, this disclosure provides a KOD polymerase variant comprising: a Y at position I18 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I18 of SEQ ID NO: 1.

### E22

In some embodiments, this disclosure provides a KOD polymerase variant comprising: a W at a position corresponding to E22 of SEQ ID NO: 1. In some embodiments, this disclosure provides a KOD polymerase variant comprising: a W at position E22 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position E22 of SEQ ID NO: 1.

### F34

In some embodiments, this disclosure provides a KOD polymerase variant comprising: a D at a position corresponding to F34 of SEQ ID NO: 1. In some embodiments, this disclosure provides a KOD polymerase variant comprising: a D at position F34 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position F34 of SEQ ID NO: 1.

### P36

In some embodiments, a KOD polymerase variant comprises: a C, D, E, F, H, I, K, L, M, N, Q, R, T, V, W, or Y at a position corresponding to P36 of SEQ ID NO: 1. In some embodiments, a KOD polymerase variant comprises: a C, D, E, F, H, I, K, L, M, N, Q, R, T, V, W, or Y at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, F, H, I, K, L, M, N, Q, R, T, V, W, or Y at position P36 of SEQ ID NO: 1.

In some embodiments, this disclosure provides a KOD polymerase variant comprising: a C at a position corresponding to P36 of SEQ ID NO: 1. In some embodiments, this disclosure provides a KOD polymerase variant comprising: a C at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position P36 of SEQ ID NO: 1.

In some embodiments, this disclosure provides a KOD polymerase variant comprising: a D at a position corresponding to P36 of SEQ ID NO: 1. In some embodiments, this disclosure provides a KOD polymerase variant comprising: a D at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position P36 of SEQ ID NO: 1.

In some embodiments, this disclosure provides a KOD polymerase variant comprising: an E at a position corresponding to P36 of SEQ ID NO: 1. In some embodiments, this disclosure provides a KOD polymerase variant comprising: an E at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position P36 of SEQ ID NO: 1.

In some embodiments, this disclosure provides a KOD polymerase variant comprising: a F at a position corresponding to P36 of SEQ ID NO: 1. In some embodiments, this disclosure provides a KOD polymerase variant comprising: a F at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position P36 of SEQ ID NO: 1.

In some embodiments, this disclosure provides a KOD polymerase variant comprising: a H at a position corresponding to P36 of SEQ ID NO: 1. In some embodiments, this disclosure provides a KOD polymerase variant comprising: a H at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position P36 of SEQ ID NO: 1.

In some embodiments, this disclosure provides a KOD polymerase variant comprising: an I at a position corresponding to P36 of SEQ ID NO: 1. In some embodiments, this disclosure provides a KOD polymerase variant comprising: an I at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position P36 of SEQ ID NO: 1.

In some embodiments, this disclosure provides a KOD polymerase variant comprising: a K at a position corresponding to P36 of SEQ ID NO: 1. In some embodiments, this disclosure provides a KOD polymerase variant comprising: a K at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position P36 of SEQ ID NO: 1.

In some embodiments, this disclosure provides a KOD polymerase variant comprising: a L at a position corresponding to P36 of SEQ ID NO: 1. In some embodiments, this disclosure provides a KOD polymerase variant comprising: a L at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position P36 of SEQ ID NO: 1.

In some embodiments, this disclosure provides a KOD polymerase variant comprising: a M at a position corresponding to P36 of SEQ ID NO: 1. In some embodiments, this disclosure provides a KOD polymerase variant comprising: a M at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position P36 of SEQ ID NO: 1.

In some embodiments, this disclosure provides a KOD polymerase variant comprising: a N at a position corresponding to P36 of SEQ ID NO: 1. In some embodiments, this disclosure provides a KOD polymerase variant comprising: a N at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position P36 of SEQ ID NO: 1.

In some embodiments, this disclosure provides a KOD polymerase variant comprising: a Q at a position corresponding to P36 of SEQ ID NO: 1. In some embodiments, this disclosure provides a KOD polymerase variant comprising: a Q at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position P36 of SEQ ID NO: 1.

In some embodiments, this disclosure provides a KOD polymerase variant comprising: a R at a position corresponding to P36 of SEQ ID NO: 1. In some embodiments, this disclosure provides a KOD polymerase variant comprising: a R at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position P36 of SEQ ID NO: 1.

In some embodiments, this disclosure provides a KOD polymerase variant comprising: a T at a position corresponding to P36 of SEQ ID NO: 1. In some embodiments, this disclosure provides a KOD polymerase variant comprising: a T at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position P36 of SEQ ID NO: 1.

In some embodiments, this disclosure provides a KOD polymerase variant comprising: a V at a position corresponding to P36 of SEQ ID NO: 1. In some embodiments, this disclosure provides a KOD polymerase variant comprising: a V at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position P36 of SEQ ID NO: 1.

In some embodiments, this disclosure provides a KOD polymerase variant comprising: a W at a position corresponding to P36 of SEQ ID NO: 1. In some embodiments, this disclosure provides a KOD polymerase variant comprising: a W at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position P36 of SEQ ID NO: 1.

In some embodiments, this disclosure provides a KOD polymerase variant comprising: a Y at a position corresponding to P36 of SEQ ID NO: 1. In some embodiments, this disclosure provides a KOD polymerase variant comprising: a Y at position P36 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position P36 of SEQ ID NO: 1.

### Y37

In some embodiments, a KOD polymerase variant comprises: an A, D, E, F, G, S, or T at a position corresponding to Y37 of SEQ ID NO: 1. In some embodiments, a KOD polymerase variant comprises: an A, D, E, F, G, S, or T at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, F, G, S, or T at position Y37 of SEQ ID NO: 1.

In some embodiments, this disclosure provides a KOD polymerase variant comprising: an A at a position corresponding to Y37 of SEQ ID NO: 1. In some embodiments, this disclosure provides a KOD polymerase variant comprising: an A at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position Y37 of SEQ ID NO: 1.

In some embodiments, this disclosure provides a KOD polymerase variant comprising: a D at a position corresponding to Y37 of SEQ ID NO: 1. In some embodiments, this disclosure provides a KOD polymerase variant comprising: a D at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y37 of SEQ ID NO: 1.

In some embodiments, this disclosure provides a KOD polymerase variant comprising: an E at a position corresponding to Y37 of SEQ ID NO: 1. In some embodiments, this disclosure provides a KOD polymerase variant comprising: an E at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y37 of SEQ ID NO: 1.

In some embodiments, this disclosure provides a KOD polymerase variant comprising: a F at a position corresponding to Y37 of SEQ ID NO: 1. In some embodiments, this disclosure provides a KOD polymerase variant comprising: a F at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position Y37 of SEQ ID NO: 1.

In some embodiments, this disclosure provides a KOD polymerase variant comprising: a G at a position corresponding to Y37 of SEQ ID NO: 1. In some embodiments, this disclosure provides a KOD polymerase variant comprising: a G at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position Y37 of SEQ ID NO: 1.

In some embodiments, this disclosure provides a KOD polymerase variant comprising: a S at a position corresponding to Y37 of SEQ ID NO: 1. In some embodiments, this disclosure provides a KOD polymerase variant comprising: a S at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position Y37 of SEQ ID NO: 1.

In some embodiments, this disclosure provides a KOD polymerase variant comprising: a T at a position corresponding to Y37 of SEQ ID NO: 1. In some embodiments, this disclosure provides a KOD polymerase variant comprising: a T at position Y37 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position Y37 of SEQ ID NO: 1.

### V71

In some embodiments, this disclosure provides a KOD polymerase variant comprising: a L at a position corresponding to V71 of SEQ ID NO: 1. In some embodiments, this disclosure provides a KOD polymerase variant comprising: a L at position V71 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position V71 of SEQ ID NO: 1.

### F87

In some embodiments, this disclosure provides a KOD polymerase variant comprising: a R at a position corresponding to F87 of SEQ ID NO: 1. In some embodiments, this disclosure provides a KOD polymerase variant comprising: a R at position F87 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position F87 of SEQ ID NO: 1.

### H89

In some embodiments, a KOD polymerase variant comprises: a D, P, or W at a position corresponding to H89 of SEQ ID NO: 1. In some embodiments, a KOD polymerase variant comprises: a D, P, or W at position H89 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, P, or W at position H89 of SEQ ID NO: 1.

In some embodiments, this disclosure provides a KOD polymerase variant comprising: a D at a position corresponding to H89 of SEQ ID NO: 1. In some embodiments, this disclosure provides a KOD polymerase variant comprising: a D at position H89 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position H89 of SEQ ID NO: 1.

In some embodiments, this disclosure provides a KOD polymerase variant comprising: a P at a position corresponding to H89 of SEQ ID NO: 1. In some embodiments, this disclosure provides a KOD polymerase variant comprising: a P at position H89 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position H89 of SEQ ID NO: 1.

In some embodiments, this disclosure provides a KOD polymerase variant comprising: a W at a position corresponding to H89 of SEQ ID NO: 1. In some embodiments, this disclosure provides a KOD polymerase variant comprising: a W at position H89 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position H89 of SEQ ID NO: 1.

### P90

In some embodiments, a KOD polymerase variant comprises: a C, D, E, F, G, H, I, K, L, M, N, Q, R, W, or Y at a position corresponding to P90 of SEQ ID NO: 1. In some embodiments, a KOD polymerase variant comprises: a C, D, E, F, G, H, I, K, L, M, N, Q, R, W, or Y at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, F, G, H, I, K, L, M, N, Q, R, W, or Y at position P90 of SEQ ID NO: 1.

In some embodiments, this disclosure provides a KOD polymerase variant comprising: a C at a position corresponding to P90 of SEQ ID NO: 1. In some embodiments, this disclosure provides a KOD polymerase variant comprising: a C at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position P90 of SEQ ID NO: 1.

In some embodiments, this disclosure provides a KOD polymerase variant comprising: a D at a position corresponding to P90 of SEQ ID NO: 1. In some embodiments, this disclosure provides a KOD polymerase variant comprising: a D at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position P90 of SEQ ID NO: 1.

In some embodiments, this disclosure provides a KOD polymerase variant comprising: an E at a position corresponding to P90 of SEQ ID NO: 1. In some embodiments, this disclosure provides a KOD polymerase variant comprising: an E at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position P90 of SEQ ID NO: 1.

In some embodiments, this disclosure provides a KOD polymerase variant comprising: a F at a position corresponding to P90 of SEQ ID NO: 1. In some embodiments, this disclosure provides a KOD polymerase variant comprising: a F at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position P90 of SEQ ID NO: 1.

In some embodiments, this disclosure provides a KOD polymerase variant comprising: a G at a position corresponding to P90 of SEQ ID NO: 1. In some embodiments, this disclosure provides a KOD polymerase variant comprising: a G at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position P90 of SEQ ID NO: 1.

In some embodiments, this disclosure provides a KOD polymerase variant comprising: a H at a position corresponding to P90 of SEQ ID NO: 1. In some embodiments, this disclosure provides a KOD polymerase variant comprising: a H at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position P90 of SEQ ID NO: 1.

In some embodiments, this disclosure provides a KOD polymerase variant comprising: an I at a position corresponding to P90 of SEQ ID NO: 1. In some embodiments, this disclosure provides a KOD polymerase variant comprising: an I at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position P90 of SEQ ID NO: 1.

In some embodiments, this disclosure provides a KOD polymerase variant comprising: a K at a position corresponding to P90 of SEQ ID NO: 1. In some embodiments, this disclosure provides a KOD polymerase variant comprising: a K at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position P90 of SEQ ID NO: 1.

In some embodiments, this disclosure provides a KOD polymerase variant comprising: a L at a position corresponding to P90 of SEQ ID NO: 1. In some embodiments, this disclosure provides a KOD polymerase variant comprising: a L at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position P90 of SEQ ID NO: 1.

In some embodiments, this disclosure provides a KOD polymerase variant comprising: a M at a position corresponding to P90 of SEQ ID NO: 1. In some embodiments, this disclosure provides a KOD polymerase variant comprising: a M at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position P90 of SEQ ID NO: 1.

In some embodiments, this disclosure provides a KOD polymerase variant comprising: a N at a position corresponding to P90 of SEQ ID NO: 1. In some embodiments, this disclosure provides a KOD polymerase variant comprising: a N at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position P90 of SEQ ID NO: 1.

In some embodiments, this disclosure provides a KOD polymerase variant comprising: a Q at a position corresponding to P90 of SEQ ID NO: 1. In some embodiments, this disclosure provides a KOD polymerase variant comprising: a Q at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position P90 of SEQ ID NO: 1.

In some embodiments, this disclosure provides a KOD polymerase variant comprising: a R at a position corresponding to P90 of SEQ ID NO: 1. In some embodiments, this disclosure provides a KOD polymerase variant comprising: a R at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position P90 of SEQ ID NO: 1.

In some embodiments, this disclosure provides a KOD polymerase variant comprising: a W at a position corresponding to P90 of SEQ ID NO: 1. In some embodiments, this disclosure provides a KOD polymerase variant comprising: a W at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position P90 of SEQ ID NO: 1.

In some embodiments, this disclosure provides a KOD polymerase variant comprising: a Y at a position corresponding to P90 of SEQ ID NO: 1. In some embodiments, this disclosure provides a KOD polymerase variant comprising: a Y at position P90 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position P90 of SEQ ID NO: 1.

### Q91

In some embodiments, a KOD polymerase variant comprises: a D, E, N, or Y at a position corresponding to Q91 of SEQ ID NO: 1. In some embodiments, a KOD polymerase variant comprises: a D, E, N, or Y at position Q91 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, N, or Y at position Q91 of SEQ ID NO: 1.

In some embodiments, this disclosure provides a KOD polymerase variant comprising: a D at a position corresponding to Q91 of SEQ ID NO: 1. In some embodiments, this disclosure provides a KOD polymerase variant comprising: a D at position Q91 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Q91 of SEQ ID NO: 1.

In some embodiments, this disclosure provides a KOD polymerase variant comprising: an E at a position corresponding to Q91 of SEQ ID NO: 1. In some embodiments, this disclosure provides a KOD polymerase variant comprising: an E at position Q91 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Q91 of SEQ ID NO: 1.

In some embodiments, this disclosure provides a KOD polymerase variant comprising: a N at a position corresponding to Q91 of SEQ ID NO: 1. In some embodiments, this disclosure provides a KOD polymerase variant comprising: a N at position Q91 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position Q91 of SEQ ID NO: 1.

In some embodiments, this disclosure provides a KOD polymerase variant comprising: a Y at a position corresponding to Q91 of SEQ ID NO: 1. In some embodiments, this disclosure provides a KOD polymerase variant comprising: a Y at position Q91 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position Q91 of SEQ ID NO: 1.

### D92

In some embodiments, this disclosure provides a KOD polymerase variant comprising: a P at a position corresponding to D92 of SEQ ID NO: 1. In some embodiments, this disclosure provides a KOD polymerase variant comprising: a P at position D92 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D92 of SEQ ID NO: 1.

### V93

In some embodiments, a KOD polymerase variant comprises: an A, C, D, E, F, G, H, K, M, N, P, Q, R, S, T, W, or Y at a position corresponding to V93 of SEQ ID NO: 1. In some embodiments, a KOD polymerase variant comprises: an A, C, D, E, F, G, H, K, M, N, P, Q, R, S, T, W, or Y at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, G, H, K, M, N, P, Q, R, S, T, W, or Y at position V93 of SEQ ID NO: 1.

In some embodiments, this disclosure provides a KOD polymerase variant comprising: an A at a position corresponding to V93 of SEQ ID NO: 1. In some embodiments, this disclosure provides a KOD polymerase variant comprising: an A at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position V93 of SEQ ID NO: 1.

In some embodiments, this disclosure provides a KOD polymerase variant comprising: a C at a position corresponding to V93 of SEQ ID NO: 1. In some embodiments, this disclosure provides a KOD polymerase variant comprising: a C at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position V93 of SEQ ID NO: 1.

In some embodiments, this disclosure provides a KOD polymerase variant comprising: a D at a position corresponding to V93 of SEQ ID NO: 1. In some embodiments, this disclosure provides a KOD polymerase variant comprising: a D at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position V93 of SEQ ID NO: 1.

In some embodiments, this disclosure provides a KOD polymerase variant comprising: an E at a position corresponding to V93 of SEQ ID NO: 1. In some embodiments, this disclosure provides a KOD polymerase variant comprising: an E at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position V93 of SEQ ID NO: 1.

In some embodiments, this disclosure provides a KOD polymerase variant comprising: a F at a position corresponding to V93 of SEQ ID NO: 1. In some embodiments, this disclosure provides a KOD polymerase variant comprising: a F at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position V93 of SEQ ID NO: 1.

In some embodiments, this disclosure provides a KOD polymerase variant comprising: a G at a position corresponding to V93 of SEQ ID NO: 1. In some embodiments, this disclosure provides a KOD polymerase variant comprising: a G at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position V93 of SEQ ID NO: 1.

In some embodiments, this disclosure provides a KOD polymerase variant comprising: a H at a position corresponding to V93 of SEQ ID NO: 1. In some embodiments, this disclosure provides a KOD polymerase variant comprising: a H at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position V93 of SEQ ID NO: 1.

In some embodiments, this disclosure provides a KOD polymerase variant comprising: a K at a position corresponding to V93 of SEQ ID NO: 1. In some embodiments, this disclosure provides a KOD polymerase variant comprising: a K at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position V93 of SEQ ID NO: 1.

In some embodiments, this disclosure provides a KOD polymerase variant comprising: a M at a position corresponding to V93 of SEQ ID NO: 1. In some embodiments, this disclosure provides a KOD polymerase variant comprising: a M at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position V93 of SEQ ID NO: 1.

In some embodiments, this disclosure provides a KOD polymerase variant comprising: a N at a position corresponding to V93 of SEQ ID NO: 1. In some embodiments, this disclosure provides a KOD polymerase variant comprising: a N at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position V93 of SEQ ID NO: 1.

In some embodiments, this disclosure provides a KOD polymerase variant comprising: a P at a position corresponding to V93 of SEQ ID NO: 1. In some embodiments, this disclosure provides a KOD polymerase variant comprising: a P at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position V93 of SEQ ID NO: 1.

In some embodiments, this disclosure provides a KOD polymerase variant comprising: a Q at a position corresponding to V93 of SEQ ID NO: 1. In some embodiments, this disclosure provides a KOD polymerase variant comprising: a Q at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position V93 of SEQ ID NO: 1.

In some embodiments, this disclosure provides a KOD polymerase variant comprising: a R at a position corresponding to V93 of SEQ ID NO: 1. In some embodiments, this disclosure provides a KOD polymerase variant comprising: a R at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position V93 of SEQ ID NO: 1.

In some embodiments, this disclosure provides a KOD polymerase variant comprising: a S at a position corresponding to V93 of SEQ ID NO: 1. In some embodiments, this disclosure provides a KOD polymerase variant comprising: a S at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position V93 of SEQ ID NO: 1.

In some embodiments, this disclosure provides a KOD polymerase variant comprising: a T at a position corresponding to V93 of SEQ ID NO: 1. In some embodiments, this disclosure provides a KOD polymerase variant comprising: a T at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position V93 of SEQ ID NO: 1.

In some embodiments, this disclosure provides a KOD polymerase variant comprising: a W at a position corresponding to V93 of SEQ ID NO: 1. In some embodiments, this disclosure provides a KOD polymerase variant comprising: a W at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position V93 of SEQ ID NO: 1.

In some embodiments, this disclosure provides a KOD polymerase variant comprising: a Y at a position corresponding to V93 of SEQ ID NO: 1. In some embodiments, this disclosure provides a KOD polymerase variant comprising: a Y at position V93 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position V93 of SEQ ID NO: 1.

### P94

In some embodiments, a KOD polymerase variant comprises: a C, D, E, F, G, H, I, M, N, Q, W, or Y at a position corresponding to P94 of SEQ ID NO: 1. In some embodiments, a KOD polymerase variant comprises: a C, D, E, F, G, H, I, M, N, Q, W, or Y at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, F, G, H, I, M, N, Q, W, or Y at position P94 of SEQ ID NO: 1.

In some embodiments, this disclosure provides a KOD polymerase variant comprising: a C at a position corresponding to P94 of SEQ ID NO: 1. In some embodiments, this disclosure provides a KOD polymerase variant comprising: a C at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position P94 of SEQ ID NO: 1.

In some embodiments, this disclosure provides a KOD polymerase variant comprising: a D at a position corresponding to P94 of SEQ ID NO: 1. In some embodiments, this disclosure provides a KOD polymerase variant comprising: a D at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position P94 of SEQ ID NO: 1.

In some embodiments, this disclosure provides a KOD polymerase variant comprising: an E at a position corresponding to P94 of SEQ ID NO: 1. In some embodiments, this disclosure provides a KOD polymerase variant comprising: an E at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position P94 of SEQ ID NO: 1.

In some embodiments, this disclosure provides a KOD polymerase variant comprising: a F at a position corresponding to P94 of SEQ ID NO: 1. In some embodiments, this disclosure provides a KOD polymerase variant comprising: a F at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position P94 of SEQ ID NO: 1.

In some embodiments, this disclosure provides a KOD polymerase variant comprising: a G at a position corresponding to P94 of SEQ ID NO: 1. In some embodiments, this disclosure provides a KOD polymerase variant comprising: a G at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position P94 of SEQ ID NO: 1.

In some embodiments, this disclosure provides a KOD polymerase variant comprising: a H at a position corresponding to P94 of SEQ ID NO: 1. In some embodiments, this disclosure provides a KOD polymerase variant comprising: a H at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position P94 of SEQ ID NO: 1.

In some embodiments, this disclosure provides a KOD polymerase variant comprising: an I at a position corresponding to P94 of SEQ ID NO: 1. In some embodiments, this disclosure provides a KOD polymerase variant comprising: an I at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position P94 of SEQ ID NO: 1.

In some embodiments, this disclosure provides a KOD polymerase variant comprising: a M at a position corresponding to P94 of SEQ ID NO: 1. In some embodiments, this disclosure provides a KOD polymerase variant comprising: a M at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position P94 of SEQ ID NO: 1.

In some embodiments, this disclosure provides a KOD polymerase variant comprising: a N at a position corresponding to P94 of SEQ ID NO: 1. In some embodiments, this disclosure provides a KOD polymerase variant comprising: a N at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position P94 of SEQ ID NO: 1.

In some embodiments, this disclosure provides a KOD polymerase variant comprising: a Q at a position corresponding to P94 of SEQ ID NO: 1. In some embodiments, this disclosure provides a KOD polymerase variant comprising: a Q at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position P94 of SEQ ID NO: 1.

In some embodiments, this disclosure provides a KOD polymerase variant comprising: a W at a position corresponding to P94 of SEQ ID NO: 1. In some embodiments, this disclosure provides a KOD polymerase variant comprising: a W at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position P94 of SEQ ID NO: 1.

In some embodiments, this disclosure provides a KOD polymerase variant comprising: a Y at a position corresponding to P94 of SEQ ID NO: 1. In some embodiments, this disclosure provides a KOD polymerase variant comprising: a Y at position P94 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position P94 of SEQ ID NO: 1.

### I96

In some embodiments, a KOD polymerase variant comprises: a P or R at a position corresponding to I96 of SEQ ID NO: 1. In some embodiments, a KOD polymerase variant comprises: a P or R at position I96 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P or R at position I96 of SEQ ID NO: 1.

In some embodiments, this disclosure provides a KOD polymerase variant comprising: a P at a position corresponding to I96 of SEQ ID NO: 1. In some embodiments, this disclosure provides a KOD polymerase variant comprising: a P at position I96 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position I96 of SEQ ID NO: 1.

In some embodiments, this disclosure provides a KOD polymerase variant comprising: a R at a position corresponding to I96 of SEQ ID NO: 1. In some embodiments, this disclosure provides a KOD polymerase variant comprising: a R at position I96 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position I96 of SEQ ID NO: 1.

### R97

In some embodiments, a KOD polymerase variant comprises: an A, C, D, E, F, G, H, I, K, L, M, N, P, Q, S, T, V, W, or Y at a position corresponding to R97 of SEQ ID NO: 1. In some embodiments, a KOD polymerase variant comprises: an A, C, D, E, F, G, H, I, K, L, M, N, P, Q, S, T, V, W, or Y at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, G, H, I, K, L, M, N, P, Q, S, T, V, W, or Y at position R97 of SEQ ID NO: 1.

In some embodiments, this disclosure provides a KOD polymerase variant comprising: an A at a position corresponding to R97 of SEQ ID NO: 1. In some embodiments, this disclosure provides a KOD polymerase variant comprising: an A at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position R97 of SEQ ID NO: 1.

In some embodiments, this disclosure provides a KOD polymerase variant comprising: a C at a position corresponding to R97 of SEQ ID NO: 1. In some embodiments, this disclosure provides a KOD polymerase variant comprising: a C at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position R97 of SEQ ID NO: 1.

In some embodiments, this disclosure provides a KOD polymerase variant comprising: a D at a position corresponding to R97 of SEQ ID NO: 1. In some embodiments, this disclosure provides a KOD polymerase variant comprising: a D at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position R97 of SEQ ID NO: 1.

In some embodiments, this disclosure provides a KOD polymerase variant comprising: an E at a position corresponding to R97 of SEQ ID NO: 1. In some embodiments, this disclosure provides a KOD polymerase variant comprising: an E at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position R97 of SEQ ID NO: 1.

In some embodiments, this disclosure provides a KOD polymerase variant comprising: a F at a position corresponding to R97 of SEQ ID NO: 1. In some embodiments, this disclosure provides a KOD polymerase variant comprising: a F at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position R97 of SEQ ID NO: 1.

In some embodiments, this disclosure provides a KOD polymerase variant comprising: a G at a position corresponding to R97 of SEQ ID NO: 1. In some embodiments, this disclosure provides a KOD polymerase variant comprising: a G at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position R97 of SEQ ID NO: 1.

In some embodiments, this disclosure provides a KOD polymerase variant comprising: a H at a position corresponding to R97 of SEQ ID NO: 1. In some embodiments, this disclosure provides a KOD polymerase variant comprising: a H at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position R97 of SEQ ID NO: 1.

In some embodiments, this disclosure provides a KOD polymerase variant comprising: an I at a position corresponding to R97 of SEQ ID NO: 1. In some embodiments, this disclosure provides a KOD polymerase variant comprising: an I at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position R97 of SEQ ID NO: 1.

In some embodiments, this disclosure provides a KOD polymerase variant comprising: a K at a position corresponding to R97 of SEQ ID NO: 1. In some embodiments, this disclosure provides a KOD polymerase variant comprising: a K at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position R97 of SEQ ID NO: 1.

In some embodiments, this disclosure provides a KOD polymerase variant comprising: a L at a position corresponding to R97 of SEQ ID NO: 1. In some embodiments, this disclosure provides a KOD polymerase variant comprising: a L at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position R97 of SEQ ID NO: 1.

In some embodiments, this disclosure provides a KOD polymerase variant comprising: a M at a position corresponding to R97 of SEQ ID NO: 1. In some embodiments, this disclosure provides a KOD polymerase variant comprising: a M at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position R97 of SEQ ID NO: 1.

In some embodiments, this disclosure provides a KOD polymerase variant comprising: a N at a position corresponding to R97 of SEQ ID NO: 1. In some embodiments, this disclosure provides a KOD polymerase variant comprising: a N at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position R97 of SEQ ID NO: 1.

In some embodiments, this disclosure provides a KOD polymerase variant comprising: a P at a position corresponding to R97 of SEQ ID NO: 1. In some embodiments, this disclosure provides a KOD polymerase variant comprising: a P at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position R97 of SEQ ID NO: 1.

In some embodiments, this disclosure provides a KOD polymerase variant comprising: a Q at a position corresponding to R97 of SEQ ID NO: 1. In some embodiments, this disclosure provides a KOD polymerase variant comprising: a Q at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position R97 of SEQ ID NO: 1.

In some embodiments, this disclosure provides a KOD polymerase variant comprising: a S at a position corresponding to R97 of SEQ ID NO: 1. In some embodiments, this disclosure provides a KOD polymerase variant comprising: a S at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position R97 of SEQ ID NO: 1.

In some embodiments, this disclosure provides a KOD polymerase variant comprising: a T at a position corresponding to R97 of SEQ ID NO: 1. In some embodiments, this disclosure provides a KOD polymerase variant comprising: a T at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position R97 of SEQ ID NO: 1.

In some embodiments, this disclosure provides a KOD polymerase variant comprising: a V at a position corresponding to R97 of SEQ ID NO: 1. In some embodiments, this disclosure provides a KOD polymerase variant comprising: a V at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position R97 of SEQ ID NO: 1.

In some embodiments, this disclosure provides a KOD polymerase variant comprising: a W at a position corresponding to R97 of SEQ ID NO: 1. In some embodiments, this disclosure provides a KOD polymerase variant comprising: a W at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position R97 of SEQ ID NO: 1.

In some embodiments, this disclosure provides a KOD polymerase variant comprising: a Y at a position corresponding to R97 of SEQ ID NO: 1. In some embodiments, this disclosure provides a KOD polymerase variant comprising: a Y at position R97 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position R97 of SEQ ID NO: 1.

### R101

In some embodiments, a KOD polymerase variant comprises: an E or Q at a position corresponding to R101 of SEQ ID NO: 1. In some embodiments, a KOD polymerase variant comprises: an E or Q at position R101 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E or Q at position R101 of SEQ ID NO: 1.

In some embodiments, this disclosure provides a KOD polymerase variant comprising: an E at a position corresponding to R101 of SEQ ID NO: 1. In some embodiments, this disclosure provides a KOD polymerase variant comprising: an E at position R101 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position R101 of SEQ ID NO: 1.

In some embodiments, this disclosure provides a KOD polymerase variant comprising: a Q at a position corresponding to R101 of SEQ ID NO: 1. In some embodiments, this disclosure provides a KOD polymerase variant comprising: a Q at position R101 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position R101 of SEQ ID NO: 1.

### I109

In some embodiments, a KOD polymerase variant comprises: a D or F at a position corresponding to I109 of SEQ ID NO: 1. In some embodiments, a KOD polymerase variant comprises: a D or F at position I109 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or F at position I109 of SEQ ID NO: 1.

In some embodiments, this disclosure provides a KOD polymerase variant comprising: a D at a position corresponding to I109 of SEQ ID NO: 1. In some embodiments, this disclosure provides a KOD polymerase variant comprising: a D at position I109 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position I109 of SEQ ID NO: 1.

In some embodiments, this disclosure provides a KOD polymerase variant comprising: a F at a position corresponding to I109 of SEQ ID NO: 1. In some embodiments, this disclosure provides a KOD polymerase variant comprising: a F at position I109 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I109 of SEQ ID NO: 1.

### Y110

In some embodiments, this disclosure provides a KOD polymerase variant comprising: a P at a position corresponding to Y110 of SEQ ID NO: 1. In some embodiments, this disclosure provides a KOD polymerase variant comprising: a P at position Y110 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y110 of SEQ ID NO: 1.

### E111

In some embodiments, this disclosure provides a KOD polymerase variant comprising: a G at a position corresponding to E111 of SEQ ID NO: 1. In some embodiments, this disclosure provides a KOD polymerase variant comprising: a G at position E111 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position E111 of SEQ ID NO: 1.

### Y112

In some embodiments, a KOD polymerase variant comprises: a D or E at a position corresponding to Y112 of SEQ ID NO: 1. In some embodiments, a KOD polymerase variant comprises: a D or E at position Y112 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or E at position Y112 of SEQ ID NO: 1.

In some embodiments, this disclosure provides a KOD polymerase variant comprising: a D at a position corresponding to Y112 of SEQ ID NO: 1. In some embodiments, this disclosure provides a KOD polymerase variant comprising: a D at position Y112 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y112 of SEQ ID NO: 1.

In some embodiments, this disclosure provides a KOD polymerase variant comprising: an E at a position corresponding to Y112 of SEQ ID NO: 1. In some embodiments, this disclosure provides a KOD polymerase variant comprising: an E at position Y112 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y112 of SEQ ID NO: 1.

### D113

In some embodiments, this disclosure provides a KOD polymerase variant comprising: a P at a position corresponding to D113 of SEQ ID NO: 1. In some embodiments, this disclosure provides a KOD polymerase variant comprising: a P at position D113 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D113 of SEQ ID NO: 1.

### I114

In some embodiments, a KOD polymerase variant comprises: an E, G, H, K, N, P, Q, S, or Y at a position corresponding to I114 of SEQ ID NO: 1. In some embodiments, a KOD polymerase variant comprises: an E, G, H, K, N, P, Q, S, or Y at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E, G, H, K, N, P, Q, S, or Y at position I114 of SEQ ID NO: 1.

In some embodiments, this disclosure provides a KOD polymerase variant comprising: an E at a position corresponding to I114 of SEQ ID NO: 1. In some embodiments, this disclosure provides a KOD polymerase variant comprising: an E at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position I114 of SEQ ID NO: 1.

In some embodiments, this disclosure provides a KOD polymerase variant comprising: a G at a position corresponding to I114 of SEQ ID NO: 1. In some embodiments, this disclosure provides a KOD polymerase variant comprising: a G at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position I114 of SEQ ID NO: 1.

In some embodiments, this disclosure provides a KOD polymerase variant comprising: a H at a position corresponding to I114 of SEQ ID NO: 1. In some embodiments, this disclosure provides a KOD polymerase variant comprising: a H at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I114 of SEQ ID NO: 1.

In some embodiments, this disclosure provides a KOD polymerase variant comprising: a K at a position corresponding to I114 of SEQ ID NO: 1. In some embodiments, this disclosure provides a KOD polymerase variant comprising: a K at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I114 of SEQ ID NO: 1.

In some embodiments, this disclosure provides a KOD polymerase variant comprising: a N at a position corresponding to I114 of SEQ ID NO: 1. In some embodiments, this disclosure provides a KOD polymerase variant comprising: a N at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position I114 of SEQ ID NO: 1.

In some embodiments, this disclosure provides a KOD polymerase variant comprising: a P at a position corresponding to I114 of SEQ ID NO: 1. In some embodiments, this disclosure provides a KOD polymerase variant comprising: a P at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position I114 of SEQ ID NO: 1.

In some embodiments, this disclosure provides a KOD polymerase variant comprising: a Q at a position corresponding to I114 of SEQ ID NO: 1. In some embodiments, this disclosure provides a KOD polymerase variant comprising: a Q at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position I114 of SEQ ID NO: 1.

In some embodiments, this disclosure provides a KOD polymerase variant comprising: a S at a position corresponding to I114 of SEQ ID NO: 1. In some embodiments, this disclosure provides a KOD polymerase variant comprising: a S at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position I114 of SEQ ID NO: 1.

In some embodiments, this disclosure provides a KOD polymerase variant comprising: a Y at a position corresponding to I114 of SEQ ID NO: 1. In some embodiments, this disclosure provides a KOD polymerase variant comprising: a Y at position I114 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I114 of SEQ ID NO: 1.

### P115

In some embodiments, a KOD polymerase variant comprises: a C, D, F, H, N, S, T, or Y at a position corresponding to P115 of SEQ ID NO: 1. In some embodiments, a KOD polymerase variant comprises: a C, D, F, H, N, S, T, or Y at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, F, H, N, S, T, or Y at position P115 of SEQ ID NO: 1.

In some embodiments, this disclosure provides a KOD polymerase variant comprising: a C at a position corresponding to P115 of SEQ ID NO: 1. In some embodiments, this disclosure provides a KOD polymerase variant comprising: a C at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position P115 of SEQ ID NO: 1.

In some embodiments, this disclosure provides a KOD polymerase variant comprising: a D at a position corresponding to P115 of SEQ ID NO: 1. In some embodiments, this disclosure provides a KOD polymerase variant comprising: a D at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position P115 of SEQ ID NO: 1.

In some embodiments, this disclosure provides a KOD polymerase variant comprising: a F at a position corresponding to P115 of SEQ ID NO: 1. In some embodiments, this disclosure provides a KOD polymerase variant comprising: a F at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position P115 of SEQ ID NO: 1.

In some embodiments, this disclosure provides a KOD polymerase variant comprising: a H at a position corresponding to P115 of SEQ ID NO: 1. In some embodiments, this disclosure provides a KOD polymerase variant comprising: a H at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position P115 of SEQ ID NO: 1.

In some embodiments, this disclosure provides a KOD polymerase variant comprising: a N at a position corresponding to P115 of SEQ ID NO: 1. In some embodiments, this disclosure provides a KOD polymerase variant comprising: a N at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position P115 of SEQ ID NO: 1.

In some embodiments, this disclosure provides a KOD polymerase variant comprising: a S at a position corresponding to P115 of SEQ ID NO: 1. In some embodiments, this disclosure provides a KOD polymerase variant comprising: a S at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position P115 of SEQ ID NO: 1.

In some embodiments, this disclosure provides a KOD polymerase variant comprising: a T at a position corresponding to P115 of SEQ ID NO: 1. In some embodiments, this disclosure provides a KOD polymerase variant comprising: a T at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position P115 of SEQ ID NO: 1.

In some embodiments, this disclosure provides a KOD polymerase variant comprising: a Y at a position corresponding to P115 of SEQ ID NO: 1. In some embodiments, this disclosure provides a KOD polymerase variant comprising: a Y at position P115 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position P115 of SEQ ID NO: 1.

### F116

In some embodiments, a KOD polymerase variant comprises: an A, D, E, G, H, I, K, N, P, Q, R, or V at a position corresponding to F116 of SEQ ID NO: 1. In some embodiments, a KOD polymerase variant comprises: an A, D, E, G, H, I, K, N, P, Q, R, or V at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, G, H, I, K, N, P, Q, R, or V at position F116 of SEQ ID NO: 1.

In some embodiments, this disclosure provides a KOD polymerase variant comprising: an A at a position corresponding to F116 of SEQ ID NO: 1. In some embodiments, this disclosure provides a KOD polymerase variant comprising: an A at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position F116 of SEQ ID NO: 1.

In some embodiments, this disclosure provides a KOD polymerase variant comprising: a D at a position corresponding to F116 of SEQ ID NO: 1. In some embodiments, this disclosure provides a KOD polymerase variant comprising: a D at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position F116 of SEQ ID NO: 1.

In some embodiments, this disclosure provides a KOD polymerase variant comprising: an E at a position corresponding to F116 of SEQ ID NO: 1. In some embodiments, this disclosure provides a KOD polymerase variant comprising: an E at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position F116 of SEQ ID NO: 1.

In some embodiments, this disclosure provides a KOD polymerase variant comprising: a G at a position corresponding to F116 of SEQ ID NO: 1. In some embodiments, this disclosure provides a KOD polymerase variant comprising: a G at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position F116 of SEQ ID NO: 1.

In some embodiments, this disclosure provides a KOD polymerase variant comprising: a H at a position corresponding to F116 of SEQ ID NO: 1. In some embodiments, this disclosure provides a KOD polymerase variant comprising: a H at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position F116 of SEQ ID NO: 1.

In some embodiments, this disclosure provides a KOD polymerase variant comprising: an I at a position corresponding to F116 of SEQ ID NO: 1. In some embodiments, this disclosure provides a KOD polymerase variant comprising: an I at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position F116 of SEQ ID NO: 1.

In some embodiments, this disclosure provides a KOD polymerase variant comprising: a K at a position corresponding to F116 of SEQ ID NO: 1. In some embodiments, this disclosure provides a KOD polymerase variant comprising: a K at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position F116 of SEQ ID NO: 1.

In some embodiments, this disclosure provides a KOD polymerase variant comprising: a N at a position corresponding to F116 of SEQ ID NO: 1. In some embodiments, this disclosure provides a KOD polymerase variant comprising: a N at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position F116 of SEQ ID NO: 1.

In some embodiments, this disclosure provides a KOD polymerase variant comprising: a P at a position corresponding to F116 of SEQ ID NO: 1. In some embodiments, this disclosure provides a KOD polymerase variant comprising: a P at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position F116 of SEQ ID NO: 1.

In some embodiments, this disclosure provides a KOD polymerase variant comprising: a Q at a position corresponding to F116 of SEQ ID NO: 1. In some embodiments, this disclosure provides a KOD polymerase variant comprising: a Q at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position F116 of SEQ ID NO: 1.

In some embodiments, this disclosure provides a KOD polymerase variant comprising: a R at a position corresponding to F116 of SEQ ID NO: 1. In some embodiments, this disclosure provides a KOD polymerase variant comprising: a R at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position F116 of SEQ ID NO: 1.

In some embodiments, this disclosure provides a KOD polymerase variant comprising: a V at a position corresponding to F116 of SEQ ID NO: 1. In some embodiments, this disclosure provides a KOD polymerase variant comprising: a V at position F116 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position F116 of SEQ ID NO: 1.

### A117

In some embodiments, a KOD polymerase variant comprises: a D, E, L, M, Q, or W at a position corresponding to A117 of SEQ ID NO: 1. In some embodiments, a KOD polymerase variant comprises: a D, E, L, M, Q, or W at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, L, M, Q, or W at position A117 of SEQ ID NO: 1.

In some embodiments, this disclosure provides a KOD polymerase variant comprising: a D at a position corresponding to A117 of SEQ ID NO: 1. In some embodiments, this disclosure provides a KOD polymerase variant comprising: a D at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A117 of SEQ ID NO: 1.

In some embodiments, this disclosure provides a KOD polymerase variant comprising: an E at a position corresponding to A117 of SEQ ID NO: 1. In some embodiments, this disclosure provides a KOD polymerase variant comprising: an E at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A117 of SEQ ID NO: 1.

In some embodiments, this disclosure provides a KOD polymerase variant comprising: a L at a position corresponding to A117 of SEQ ID NO: 1. In some embodiments, this disclosure provides a KOD polymerase variant comprising: a L at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position A117 of SEQ ID NO: 1.

In some embodiments, this disclosure provides a KOD polymerase variant comprising: a M at a position corresponding to A117 of SEQ ID NO: 1. In some embodiments, this disclosure provides a KOD polymerase variant comprising: a M at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position A117 of SEQ ID NO: 1.

In some embodiments, this disclosure provides a KOD polymerase variant comprising: a Q at a position corresponding to A117 of SEQ ID NO: 1. In some embodiments, this disclosure provides a KOD polymerase variant comprising: a Q at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position A117 of SEQ ID NO: 1.

In some embodiments, this disclosure provides a KOD polymerase variant comprising: a W at a position corresponding to A117 of SEQ ID NO: 1. In some embodiments, this disclosure provides a KOD polymerase variant comprising: a W at position A117 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position A117 of SEQ ID NO: 1.

### R119

In some embodiments, a KOD polymerase variant comprises: an A, C, D, E, F, G, H, I, K, L, M, N, Q, S, T, V, W, or Y at a position corresponding to R119 of SEQ ID NO: 1. In some embodiments, a KOD polymerase variant comprises: an A, C, D, E, F, G, H, I, K, L, M, N, Q, S, T, V, W, or Y at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, G, H, I, K, L, M, N, Q, S, T, V, W, or Y at position R119 of SEQ ID NO: 1.

In some embodiments, this disclosure provides a KOD polymerase variant comprising: an A at a position corresponding to R119 of SEQ ID NO: 1. In some embodiments, this disclosure provides a KOD polymerase variant comprising: an A at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position R119 of SEQ ID NO: 1.

In some embodiments, this disclosure provides a KOD polymerase variant comprising: a C at a position corresponding to R119 of SEQ ID NO: 1. In some embodiments, this disclosure provides a KOD polymerase variant comprising: a C at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position R119 of SEQ ID NO: 1.

In some embodiments, this disclosure provides a KOD polymerase variant comprising: a D at a position corresponding to R119 of SEQ ID NO: 1. In some embodiments, this disclosure provides a KOD polymerase variant comprising: a D at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position R119 of SEQ ID NO: 1.

In some embodiments, this disclosure provides a KOD polymerase variant comprising: an E at a position corresponding to R119 of SEQ ID NO: 1. In some embodiments, this disclosure provides a KOD polymerase variant comprising: an E at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position R119 of SEQ ID NO: 1.

In some embodiments, this disclosure provides a KOD polymerase variant comprising: a F at a position corresponding to R119 of SEQ ID NO: 1. In some embodiments, this disclosure provides a KOD polymerase variant comprising: a F at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position R119 of SEQ ID NO: 1.

In some embodiments, this disclosure provides a KOD polymerase variant comprising: a G at a position corresponding to R119 of SEQ ID NO: 1. In some embodiments, this disclosure provides a KOD polymerase variant comprising: a G at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position R119 of SEQ ID NO: 1.

In some embodiments, this disclosure provides a KOD polymerase variant comprising: a H at a position corresponding to R119 of SEQ ID NO: 1. In some embodiments, this disclosure provides a KOD polymerase variant comprising: a H at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position R119 of SEQ ID NO: 1.

In some embodiments, this disclosure provides a KOD polymerase variant comprising: an I at a position corresponding to R119 of SEQ ID NO: 1. In some embodiments, this disclosure provides a KOD polymerase variant comprising: an I at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position R119 of SEQ ID NO: 1.

In some embodiments, this disclosure provides a KOD polymerase variant comprising: a K at a position corresponding to R119 of SEQ ID NO: 1. In some embodiments, this disclosure provides a KOD polymerase variant comprising: a K at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position R119 of SEQ ID NO: 1.

In some embodiments, this disclosure provides a KOD polymerase variant comprising: a L at a position corresponding to R119 of SEQ ID NO: 1. In some embodiments, this disclosure provides a KOD polymerase variant comprising: a L at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position R119 of SEQ ID NO: 1.

In some embodiments, this disclosure provides a KOD polymerase variant comprising: a M at a position corresponding to R119 of SEQ ID NO: 1. In some embodiments, this disclosure provides a KOD polymerase variant comprising: a M at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position R119 of SEQ ID NO: 1.

In some embodiments, this disclosure provides a KOD polymerase variant comprising: a N at a position corresponding to R119 of SEQ ID NO: 1. In some embodiments, this disclosure provides a KOD polymerase variant comprising: a N at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position R119 of SEQ ID NO: 1.

In some embodiments, this disclosure provides a KOD polymerase variant comprising: a Q at a position corresponding to R119 of SEQ ID NO: 1. In some embodiments, this disclosure provides a KOD polymerase variant comprising: a Q at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position R119 of SEQ ID NO: 1.

In some embodiments, this disclosure provides a KOD polymerase variant comprising: a S at a position corresponding to R119 of SEQ ID NO: 1. In some embodiments, this disclosure provides a KOD polymerase variant comprising: a S at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position R119 of SEQ ID NO: 1.

In some embodiments, this disclosure provides a KOD polymerase variant comprising: a T at a position corresponding to R119 of SEQ ID NO: 1. In some embodiments, this disclosure provides a KOD polymerase variant comprising: a T at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position R119 of SEQ ID NO: 1.

In some embodiments, this disclosure provides a KOD polymerase variant comprising: a V at a position corresponding to R119 of SEQ ID NO: 1. In some embodiments, this disclosure provides a KOD polymerase variant comprising: a V at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position R119 of SEQ ID NO: 1.

In some embodiments, this disclosure provides a KOD polymerase variant comprising: a W at a position corresponding to R119 of SEQ ID NO: 1. In some embodiments, this disclosure provides a KOD polymerase variant comprising: a W at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position R119 of SEQ ID NO: 1.

In some embodiments, this disclosure provides a KOD polymerase variant comprising: a Y at a position corresponding to R119 of SEQ ID NO: 1. In some embodiments, this disclosure provides a KOD polymerase variant comprising: a Y at position R119 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position R119 of SEQ ID NO: 1.

### Y120

In some embodiments, this disclosure provides a KOD polymerase variant comprising: a P at a position corresponding to Y120 of SEQ ID NO: 1. In some embodiments, this disclosure provides a KOD polymerase variant comprising: a P at position Y120 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y120 of SEQ ID NO: 1.

### I122

In some embodiments, a KOD polymerase variant comprises: a K, R, or W at a position corresponding to I122 of SEQ ID NO: 1. In some embodiments, a KOD polymerase variant comprises: a K, R, or W at position I122 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K, R, or W at position I122 of SEQ ID NO: 1.

In some embodiments, this disclosure provides a KOD polymerase variant comprising: a K at a position corresponding to I122 of SEQ ID NO: 1. In some embodiments, this disclosure provides a KOD polymerase variant comprising: a K at position I122 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I122 of SEQ ID NO: 1.

In some embodiments, this disclosure provides a KOD polymerase variant comprising: a R at a position corresponding to I122 of SEQ ID NO: 1. In some embodiments, this disclosure provides a KOD polymerase variant comprising: a R at position I122 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position I122 of SEQ ID NO: 1.

In some embodiments, this disclosure provides a KOD polymerase variant comprising: a W at a position corresponding to I122 of SEQ ID NO: 1. In some embodiments, this disclosure provides a KOD polymerase variant comprising: a W at position I122 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I122 of SEQ ID NO: 1.

### D123

In some embodiments, a KOD polymerase variant comprises: an A, C, F, H, I, K, L, M, Q, R, S, T, V, W, or Y at a position corresponding to D123 of SEQ ID NO: 1. In some embodiments, a KOD polymerase variant comprises: an A, C, F, H, I, K, L, M, Q, R, S, T, V, W, or Y at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, H, I, K, L, M, Q, R, S, T, V, W, or Y at position D123 of SEQ ID NO: 1.

In some embodiments, this disclosure provides a KOD polymerase variant comprising: an A at a position corresponding to D123 of SEQ ID NO: 1. In some embodiments, this disclosure provides a KOD polymerase variant comprising: an A at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position D123 of SEQ ID NO: 1.

In some embodiments, this disclosure provides a KOD polymerase variant comprising: a C at a position corresponding to D123 of SEQ ID NO: 1. In some embodiments, this disclosure provides a KOD polymerase variant comprising: a C at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position D123 of SEQ ID NO: 1.

In some embodiments, this disclosure provides a KOD polymerase variant comprising: a F at a position corresponding to D123 of SEQ ID NO: 1. In some embodiments, this disclosure provides a KOD polymerase variant comprising: a F at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position D123 of SEQ ID NO: 1.

In some embodiments, this disclosure provides a KOD polymerase variant comprising: a H at a position corresponding to D123 of SEQ ID NO: 1. In some embodiments, this disclosure provides a KOD polymerase variant comprising: a H at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position D123 of SEQ ID NO: 1.

In some embodiments, this disclosure provides a KOD polymerase variant comprising: an I at a position corresponding to D123 of SEQ ID NO: 1. In some embodiments, this disclosure provides a KOD polymerase variant comprising: an I at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D123 of SEQ ID NO: 1.

In some embodiments, this disclosure provides a KOD polymerase variant comprising: a K at a position corresponding to D123 of SEQ ID NO: 1. In some embodiments, this disclosure provides a KOD polymerase variant comprising: a K at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D123 of SEQ ID NO: 1.

In some embodiments, this disclosure provides a KOD polymerase variant comprising: a L at a position corresponding to D123 of SEQ ID NO: 1. In some embodiments, this disclosure provides a KOD polymerase variant comprising: a L at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D123 of SEQ ID NO: 1.

In some embodiments, this disclosure provides a KOD polymerase variant comprising: a M at a position corresponding to D123 of SEQ ID NO: 1. In some embodiments, this disclosure provides a KOD polymerase variant comprising: a M at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D123 of SEQ ID NO: 1.

In some embodiments, this disclosure provides a KOD polymerase variant comprising: a Q at a position corresponding to D123 of SEQ ID NO: 1. In some embodiments, this disclosure provides a KOD polymerase variant comprising: a Q at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D123 of SEQ ID NO: 1.

In some embodiments, this disclosure provides a KOD polymerase variant comprising: a R at a position corresponding to D123 of SEQ ID NO: 1. In some embodiments, this disclosure provides a KOD polymerase variant comprising: a R at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position D123 of SEQ ID NO: 1.

In some embodiments, this disclosure provides a KOD polymerase variant comprising: a S at a position corresponding to D123 of SEQ ID NO: 1. In some embodiments, this disclosure provides a KOD polymerase variant comprising: a S at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position D123 of SEQ ID NO: 1.

In some embodiments, this disclosure provides a KOD polymerase variant comprising: a T at a position corresponding to D123 of SEQ ID NO: 1. In some embodiments, this disclosure provides a KOD polymerase variant comprising: a T at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D123 of SEQ ID NO: 1.

In some embodiments, this disclosure provides a KOD polymerase variant comprising: a V at a position corresponding to D123 of SEQ ID NO: 1. In some embodiments, this disclosure provides a KOD polymerase variant comprising: a V at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D123 of SEQ ID NO: 1.

In some embodiments, this disclosure provides a KOD polymerase variant comprising: a W at a position corresponding to D123 of SEQ ID NO: 1. In some embodiments, this disclosure provides a KOD polymerase variant comprising: a W at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position D123 of SEQ ID NO: 1.

In some embodiments, this disclosure provides a KOD polymerase variant comprising: a Y at a position corresponding to D123 of SEQ ID NO: 1. In some embodiments, this disclosure provides a KOD polymerase variant comprising: a Y at position D123 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D123 of SEQ ID NO: 1.

### L138

In some embodiments, this disclosure provides a KOD polymerase variant comprising: a N at a position corresponding to L138 of SEQ ID NO: 1. In some embodiments, this disclosure provides a KOD polymerase variant comprising: a N at position L138 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position L138 of SEQ ID NO: 1.

### A139

In some embodiments, a KOD polymerase variant comprises: an E or Q at a position corresponding to A139 of SEQ ID NO: 1. In some embodiments, a KOD polymerase variant comprises: an E or Q at position A139 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E or Q at position A139 of SEQ ID NO: 1.

In some embodiments, this disclosure provides a KOD polymerase variant comprising: an E at a position corresponding to A139 of SEQ ID NO: 1. In some embodiments, this disclosure provides a KOD polymerase variant comprising: an E at position A139 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A139 of SEQ ID NO: 1.

In some embodiments, this disclosure provides a KOD polymerase variant comprising: a Q at a position corresponding to A139 of SEQ ID NO: 1. In some embodiments, this disclosure provides a KOD polymerase variant comprising: a Q at position A139 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position A139 of SEQ ID NO: 1.

### F140

In some embodiments, this disclosure provides a KOD polymerase variant comprising: a R at a position corresponding to F140 of SEQ ID NO: 1. In some embodiments, this disclosure provides a KOD polymerase variant comprising: a R at position F140 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position F140 of SEQ ID NO: 1.

### D141

In some embodiments, a KOD polymerase variant comprises: an A, C, G, H, K, N, P, Q, S, T, or V at a position corresponding to D141 of SEQ ID NO: 1. In some embodiments, a KOD polymerase variant comprises: an A, C, G, H, K, N, P, Q, S, T, or V at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, G, H, K, N, P, Q, S, T, or V at position D141 of SEQ ID NO: 1.

In some embodiments, this disclosure provides a KOD polymerase variant comprising: an A at a position corresponding to D141 of SEQ ID NO: 1. In some embodiments, this disclosure provides a KOD polymerase variant comprising: an A at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position D141 of SEQ ID NO: 1.

In some embodiments, this disclosure provides a KOD polymerase variant comprising: a C at a position corresponding to D141 of SEQ ID NO: 1. In some embodiments, this disclosure provides a KOD polymerase variant comprising: a C at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position D141 of SEQ ID NO: 1.

In some embodiments, this disclosure provides a KOD polymerase variant comprising: a G at a position corresponding to D141 of SEQ ID NO: 1. In some embodiments, this disclosure provides a KOD polymerase variant comprising: a G at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position D141 of SEQ ID NO: 1.

In some embodiments, this disclosure provides a KOD polymerase variant comprising: a H at a position corresponding to D141 of SEQ ID NO: 1. In some embodiments, this disclosure provides a KOD polymerase variant comprising: a H at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position D141 of SEQ ID NO: 1.

In some embodiments, this disclosure provides a KOD polymerase variant comprising: a K at a position corresponding to D141 of SEQ ID NO: 1. In some embodiments, this disclosure provides a KOD polymerase variant comprising: a K at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D141 of SEQ ID NO: 1.

In some embodiments, this disclosure provides a KOD polymerase variant comprising: a N at a position corresponding to D141 of SEQ ID NO: 1. In some embodiments, this disclosure provides a KOD polymerase variant comprising: a N at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position D141 of SEQ ID NO: 1.

In some embodiments, this disclosure provides a KOD polymerase variant comprising: a P at a position corresponding to D141 of SEQ ID NO: 1. In some embodiments, this disclosure provides a KOD polymerase variant comprising: a P at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D141 of SEQ ID NO: 1.

In some embodiments, this disclosure provides a KOD polymerase variant comprising: a Q at a position corresponding to D141 of SEQ ID NO: 1. In some embodiments, this disclosure provides a KOD polymerase variant comprising: a Q at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D141 of SEQ ID NO: 1.

In some embodiments, this disclosure provides a KOD polymerase variant comprising: a S at a position corresponding to D141 of SEQ ID NO: 1. In some embodiments, this disclosure provides a KOD polymerase variant comprising: a S at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position D141 of SEQ ID NO: 1.

In some embodiments, this disclosure provides a KOD polymerase variant comprising: a T at a position corresponding to D141 of SEQ ID NO: 1. In some embodiments, this disclosure provides a KOD polymerase variant comprising: a T at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D141 of SEQ ID NO: 1.

In some embodiments, this disclosure provides a KOD polymerase variant comprising: a V at a position corresponding to D141 of SEQ ID NO: 1. In some embodiments, this disclosure provides a KOD polymerase variant comprising: a V at position D141 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D141 of SEQ ID NO: 1.

### I142

In some embodiments, a KOD polymerase variant comprises: a F, G, H, K, P, R, W, or Y at a position corresponding to I142 of SEQ ID NO: 1. In some embodiments, a KOD polymerase variant comprises: a F, G, H, K, P, R, W, or Y at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F, G, H, K, P, R, W, or Y at position I142 of SEQ ID NO: 1.

In some embodiments, this disclosure provides a KOD polymerase variant comprising: a F at a position corresponding to I142 of SEQ ID NO: 1. In some embodiments, this disclosure provides a KOD polymerase variant comprising: a F at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I142 of SEQ ID NO: 1.

In some embodiments, this disclosure provides a KOD polymerase variant comprising: a G at a position corresponding to I142 of SEQ ID NO: 1. In some embodiments, this disclosure provides a KOD polymerase variant comprising: a G at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position I142 of SEQ ID NO: 1.

In some embodiments, this disclosure provides a KOD polymerase variant comprising: a H at a position corresponding to I142 of SEQ ID NO: 1. In some embodiments, this disclosure provides a KOD polymerase variant comprising: a H at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I142 of SEQ ID NO: 1.

In some embodiments, this disclosure provides a KOD polymerase variant comprising: a K at a position corresponding to I142 of SEQ ID NO: 1. In some embodiments, this disclosure provides a KOD polymerase variant comprising: a K at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I142 of SEQ ID NO: 1.

In some embodiments, this disclosure provides a KOD polymerase variant comprising: a P at a position corresponding to I142 of SEQ ID NO: 1. In some embodiments, this disclosure provides a KOD polymerase variant comprising: a P at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position I142 of SEQ ID NO: 1.

In some embodiments, this disclosure provides a KOD polymerase variant comprising: a R at a position corresponding to I142 of SEQ ID NO: 1. In some embodiments, this disclosure provides a KOD polymerase variant comprising: a R at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position I142 of SEQ ID NO: 1.

In some embodiments, this disclosure provides a KOD polymerase variant comprising: a W at a position corresponding to I142 of SEQ ID NO: 1. In some embodiments, this disclosure provides a KOD polymerase variant comprising: a W at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I142 of SEQ ID NO: 1.

In some embodiments, this disclosure provides a KOD polymerase variant comprising: a Y at a position corresponding to I142 of SEQ ID NO: 1. In some embodiments, this disclosure provides a KOD polymerase variant comprising: a Y at position I142 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I142 of SEQ ID NO: 1.

### E143

In some embodiments, a KOD polymerase variant comprises: an A, C, F, G, H, I, K, L, M, N, P, Q, R, S, T, V, W, or Y at a position corresponding to E143 of SEQ ID NO: 1. In some embodiments, a KOD polymerase variant comprises: an A, C, F, G, H, I, K, L, M, N, P, Q, R, S, T, V, W, or Y at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, G, H, I, K, L, M, N, P, Q, R, S, T, V, W, or Y at position E143 of SEQ ID NO: 1.

In some embodiments, this disclosure provides a KOD polymerase variant comprising: an A at a position corresponding to E143 of SEQ ID NO: 1. In some embodiments, this disclosure provides a KOD polymerase variant comprising: an A at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position E143 of SEQ ID NO: 1.

In some embodiments, this disclosure provides a KOD polymerase variant comprising: a C at a position corresponding to E143 of SEQ ID NO: 1. In some embodiments, this disclosure provides a KOD polymerase variant comprising: a C at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position E143 of SEQ ID NO: 1.

In some embodiments, this disclosure provides a KOD polymerase variant comprising: a F at a position corresponding to E143 of SEQ ID NO: 1. In some embodiments, this disclosure provides a KOD polymerase variant comprising: a F at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position E143 of SEQ ID NO: 1.

In some embodiments, this disclosure provides a KOD polymerase variant comprising: a G at a position corresponding to E143 of SEQ ID NO: 1. In some embodiments, this disclosure provides a KOD polymerase variant comprising: a G at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position E143 of SEQ ID NO: 1.

In some embodiments, this disclosure provides a KOD polymerase variant comprising: a H at a position corresponding to E143 of SEQ ID NO: 1. In some embodiments, this disclosure provides a KOD polymerase variant comprising: a H at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position E143 of SEQ ID NO: 1.

In some embodiments, this disclosure provides a KOD polymerase variant comprising: an I at a position corresponding to E143 of SEQ ID NO: 1. In some embodiments, this disclosure provides a KOD polymerase variant comprising: an I at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position E143 of SEQ ID NO: 1.

In some embodiments, this disclosure provides a KOD polymerase variant comprising: a K at a position corresponding to E143 of SEQ ID NO: 1. In some embodiments, this disclosure provides a KOD polymerase variant comprising: a K at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position E143 of SEQ ID NO: 1.

In some embodiments, this disclosure provides a KOD polymerase variant comprising: a L at a position corresponding to E143 of SEQ ID NO: 1. In some embodiments, this disclosure provides a KOD polymerase variant comprising: a L at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position E143 of SEQ ID NO: 1.

In some embodiments, this disclosure provides a KOD polymerase variant comprising: a M at a position corresponding to E143 of SEQ ID NO: 1. In some embodiments, this disclosure provides a KOD polymerase variant comprising: a M at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position E143 of SEQ ID NO: 1.

In some embodiments, this disclosure provides a KOD polymerase variant comprising: a N at a position corresponding to E143 of SEQ ID NO: 1. In some embodiments, this disclosure provides a KOD polymerase variant comprising: a N at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position E143 of SEQ ID NO: 1.

In some embodiments, this disclosure provides a KOD polymerase variant comprising: a P at a position corresponding to E143 of SEQ ID NO: 1. In some embodiments, this disclosure provides a KOD polymerase variant comprising: a P at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E143 of SEQ ID NO: 1.

In some embodiments, this disclosure provides a KOD polymerase variant comprising: a Q at a position corresponding to E143 of SEQ ID NO: 1. In some embodiments, this disclosure provides a KOD polymerase variant comprising: a Q at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position E143 of SEQ ID NO: 1.

In some embodiments, this disclosure provides a KOD polymerase variant comprising: a R at a position corresponding to E143 of SEQ ID NO: 1. In some embodiments, this disclosure provides a KOD polymerase variant comprising: a R at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position E143 of SEQ ID NO: 1.

In some embodiments, this disclosure provides a KOD polymerase variant comprising: a S at a position corresponding to E143 of SEQ ID NO: 1. In some embodiments, this disclosure provides a KOD polymerase variant comprising: a S at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position E143 of SEQ ID NO: 1.

In some embodiments, this disclosure provides a KOD polymerase variant comprising: a T at a position corresponding to E143 of SEQ ID NO: 1. In some embodiments, this disclosure provides a KOD polymerase variant comprising: a T at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position E143 of SEQ ID NO: 1.

In some embodiments, this disclosure provides a KOD polymerase variant comprising: a V at a position corresponding to E143 of SEQ ID NO: 1. In some embodiments, this disclosure provides a KOD polymerase variant comprising: a V at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position E143 of SEQ ID NO: 1.

In some embodiments, this disclosure provides a KOD polymerase variant comprising: a W at a position corresponding to E143 of SEQ ID NO: 1. In some embodiments, this disclosure provides a KOD polymerase variant comprising: a W at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position E143 of SEQ ID NO: 1.

In some embodiments, this disclosure provides a KOD polymerase variant comprising: a Y at a position corresponding to E143 of SEQ ID NO: 1. In some embodiments, this disclosure provides a KOD polymerase variant comprising: a Y at position E143 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position E143 of SEQ ID NO: 1.

### T144

In some embodiments, a KOD polymerase variant comprises: an A, D, E, F, G, H, K, L, M, P, Q, R, W, or Y at a position corresponding to T144 of SEQ ID NO: 1. In some embodiments, a KOD polymerase variant comprises: an A, D, E, F, G, H, K, L, M, P, Q, R, W, or Y at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, F, G, H, K, L, M, P, Q, R, W, or Y at position T144 of SEQ ID NO: 1.

In some embodiments, this disclosure provides a KOD polymerase variant comprising: an A at a position corresponding to T144 of SEQ ID NO: 1. In some embodiments, this disclosure provides a KOD polymerase variant comprising: an A at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position T144 of SEQ ID NO: 1.

In some embodiments, this disclosure provides a KOD polymerase variant comprising: a D at a position corresponding to T144 of SEQ ID NO: 1. In some embodiments, this disclosure provides a KOD polymerase variant comprising: a D at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position T144 of SEQ ID NO: 1.

In some embodiments, this disclosure provides a KOD polymerase variant comprising: an E at a position corresponding to T144 of SEQ ID NO: 1. In some embodiments, this disclosure provides a KOD polymerase variant comprising: an E at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position T144 of SEQ ID NO: 1.

In some embodiments, this disclosure provides a KOD polymerase variant comprising: a F at a position corresponding to T144 of SEQ ID NO: 1. In some embodiments, this disclosure provides a KOD polymerase variant comprising: a F at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position T144 of SEQ ID NO: 1.

In some embodiments, this disclosure provides a KOD polymerase variant comprising: a G at a position corresponding to T144 of SEQ ID NO: 1. In some embodiments, this disclosure provides a KOD polymerase variant comprising: a G at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position T144 of SEQ ID NO: 1.

In some embodiments, this disclosure provides a KOD polymerase variant comprising: a H at a position corresponding to T144 of SEQ ID NO: 1. In some embodiments, this disclosure provides a KOD polymerase variant comprising: a H at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position T144 of SEQ ID NO: 1.

In some embodiments, this disclosure provides a KOD polymerase variant comprising: a K at a position corresponding to T144 of SEQ ID NO: 1. In some embodiments, this disclosure provides a KOD polymerase variant comprising: a K at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position T144 of SEQ ID NO: 1.

In some embodiments, this disclosure provides a KOD polymerase variant comprising: a L at a position corresponding to T144 of SEQ ID NO: 1. In some embodiments, this disclosure provides a KOD polymerase variant comprising: a L at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position T144 of SEQ ID NO: 1.

In some embodiments, this disclosure provides a KOD polymerase variant comprising: a M at a position corresponding to T144 of SEQ ID NO: 1. In some embodiments, this disclosure provides a KOD polymerase variant comprising: a M at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position T144 of SEQ ID NO: 1.

In some embodiments, this disclosure provides a KOD polymerase variant comprising: a P at a position corresponding to T144 of SEQ ID NO: 1. In some embodiments, this disclosure provides a KOD polymerase variant comprising: a P at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position T144 of SEQ ID NO: 1.

In some embodiments, this disclosure provides a KOD polymerase variant comprising: a Q at a position corresponding to T144 of SEQ ID NO: 1. In some embodiments, this disclosure provides a KOD polymerase variant comprising: a Q at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position T144 of SEQ ID NO: 1.

In some embodiments, this disclosure provides a KOD polymerase variant comprising: a R at a position corresponding to T144 of SEQ ID NO: 1. In some embodiments, this disclosure provides a KOD polymerase variant comprising: a R at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position T144 of SEQ ID NO: 1.

In some embodiments, this disclosure provides a KOD polymerase variant comprising: a W at a position corresponding to T144 of SEQ ID NO: 1. In some embodiments, this disclosure provides a KOD polymerase variant comprising: a W at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position T144 of SEQ ID NO: 1.

In some embodiments, this disclosure provides a KOD polymerase variant comprising: a Y at a position corresponding to T144 of SEQ ID NO: 1. In some embodiments, this disclosure provides a KOD polymerase variant comprising: a Y at position T144 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position T144 of SEQ ID NO: 1.

### L145

In some embodiments, this disclosure provides a KOD polymerase variant comprising: a G at a position corresponding to L145 of SEQ ID NO: 1. In some embodiments, this disclosure provides a KOD polymerase variant comprising: a G at position L145 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position L145 of SEQ ID NO: 1.

### Y146

In some embodiments, a KOD polymerase variant comprises: a D or E at a position corresponding to Y146 of SEQ ID NO: 1. In some embodiments, a KOD polymerase variant comprises: a D or E at position Y146 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or E at position Y146 of SEQ ID NO: 1.

In some embodiments, this disclosure provides a KOD polymerase variant comprising: a D at a position corresponding to Y146 of SEQ ID NO: 1. In some embodiments, this disclosure provides a KOD polymerase variant comprising: a D at position Y146 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y146 of SEQ ID NO: 1.

In some embodiments, this disclosure provides a KOD polymerase variant comprising: an E at a position corresponding to Y146 of SEQ ID NO: 1. In some embodiments, this disclosure provides a KOD polymerase variant comprising: an E at position Y146 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y146 of SEQ ID NO: 1.

### G149

In some embodiments, this disclosure provides a KOD polymerase variant comprising: a L at a position corresponding to G149 of SEQ ID NO: 1. In some embodiments, this disclosure provides a KOD polymerase variant comprising: a L at position G149 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position G149 of SEQ ID NO: 1.

### F152

In some embodiments, a KOD polymerase variant comprises: an A, D, E, N, or S at a position corresponding to F152 of SEQ ID NO: 1. In some embodiments, a KOD polymerase variant comprises: an A, D, E, N, or S at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, N, or S at position F152 of SEQ ID NO: 1.

In some embodiments, this disclosure provides a KOD polymerase variant comprising: an A at a position corresponding to F152 of SEQ ID NO: 1. In some embodiments, this disclosure provides a KOD polymerase variant comprising: an A at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position F152 of SEQ ID NO: 1.

In some embodiments, this disclosure provides a KOD polymerase variant comprising: a D at a position corresponding to F152 of SEQ ID NO: 1. In some embodiments, this disclosure provides a KOD polymerase variant comprising: a D at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position F152 of SEQ ID NO: 1.

In some embodiments, this disclosure provides a KOD polymerase variant comprising: an E at a position corresponding to F152 of SEQ ID NO: 1. In some embodiments, this disclosure provides a KOD polymerase variant comprising: an E at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position F152 of SEQ ID NO: 1.

In some embodiments, this disclosure provides a KOD polymerase variant comprising: a N at a position corresponding to F152 of SEQ ID NO: 1. In some embodiments, this disclosure provides a KOD polymerase variant comprising: a N at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position F152 of SEQ ID NO: 1.

In some embodiments, this disclosure provides a KOD polymerase variant comprising: a S at a position corresponding to F152 of SEQ ID NO: 1. In some embodiments, this disclosure provides a KOD polymerase variant comprising: a S at position F152 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position F152 of SEQ ID NO: 1.

### E154

In some embodiments, a KOD polymerase variant comprises: a P or Y at a position corresponding to E154 of SEQ ID NO: 1. In some embodiments, a KOD polymerase variant comprises: a P or Y at position E154 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P or Y at position E154 of SEQ ID NO: 1.

In some embodiments, this disclosure provides a KOD polymerase variant comprising: a P at a position corresponding to E154 of SEQ ID NO: 1. In some embodiments, this disclosure provides a KOD polymerase variant comprising: a P at position E154 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E154 of SEQ ID NO: 1.

In some embodiments, this disclosure provides a KOD polymerase variant comprising: a Y at a position corresponding to E154 of SEQ ID NO: 1. In some embodiments, this disclosure provides a KOD polymerase variant comprising: a Y at position E154 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position E154 of SEQ ID NO: 1.

### G155

In some embodiments, a KOD polymerase variant comprises: an I or V at a position corresponding to G155 of SEQ ID NO: 1. In some embodiments, a KOD polymerase variant comprises: an I or V at position G155 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I or V at position G155 of SEQ ID NO: 1.

In some embodiments, this disclosure provides a KOD polymerase variant comprising: an I at a position corresponding to G155 of SEQ ID NO: 1. In some embodiments, this disclosure provides a KOD polymerase variant comprising: an I at position G155 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position G155 of SEQ ID NO: 1.

In some embodiments, this disclosure provides a KOD polymerase variant comprising: a V at a position corresponding to G155 of SEQ ID NO: 1. In some embodiments, this disclosure provides a KOD polymerase variant comprising: a V at position G155 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position G155 of SEQ ID NO: 1.

### I157

In some embodiments, a KOD polymerase variant comprises: an E, F, R, W, or Y at a position corresponding to I157 of SEQ ID NO: 1. In some embodiments, a KOD polymerase variant comprises: an E, F, R, W, or Y at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E, F, R, W, or Y at position I157 of SEQ ID NO: 1.

In some embodiments, this disclosure provides a KOD polymerase variant comprising: an E at a position corresponding to I157 of SEQ ID NO: 1. In some embodiments, this disclosure provides a KOD polymerase variant comprising: an E at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position I157 of SEQ ID NO: 1.

In some embodiments, this disclosure provides a KOD polymerase variant comprising: a F at a position corresponding to I157 of SEQ ID NO: 1. In some embodiments, this disclosure provides a KOD polymerase variant comprising: a F at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I157 of SEQ ID NO: 1.

In some embodiments, this disclosure provides a KOD polymerase variant comprising: a R at a position corresponding to I157 of SEQ ID NO: 1. In some embodiments, this disclosure provides a KOD polymerase variant comprising: a R at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position I157 of SEQ ID NO: 1.

In some embodiments, this disclosure provides a KOD polymerase variant comprising: a W at a position corresponding to I157 of SEQ ID NO: 1. In some embodiments, this disclosure provides a KOD polymerase variant comprising: a W at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I157 of SEQ ID NO: 1.

In some embodiments, this disclosure provides a KOD polymerase variant comprising: a Y at a position corresponding to I157 of SEQ ID NO: 1. In some embodiments, this disclosure provides a KOD polymerase variant comprising: a Y at position I157 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I157 of SEQ ID NO: 1.

### L158

In some embodiments, a KOD polymerase variant comprises: a D, H, K, Q, R, W, or Y at a position corresponding to L158 of SEQ ID NO: 1. In some embodiments, a KOD polymerase variant comprises: a D, H, K, Q, R, W, or Y at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, H, K, Q, R, W, or Y at position L158 of SEQ ID NO: 1.

In some embodiments, this disclosure provides a KOD polymerase variant comprising: a D at a position corresponding to L158 of SEQ ID NO: 1. In some embodiments, this disclosure provides a KOD polymerase variant comprising: a D at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position L158 of SEQ ID NO: 1.

In some embodiments, this disclosure provides a KOD polymerase variant comprising: a H at a position corresponding to L158 of SEQ ID NO: 1. In some embodiments, this disclosure provides a KOD polymerase variant comprising: a H at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position L158 of SEQ ID NO: 1.

In some embodiments, this disclosure provides a KOD polymerase variant comprising: a K at a position corresponding to L158 of SEQ ID NO: 1. In some embodiments, this disclosure provides a KOD polymerase variant comprising: a K at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position L158 of SEQ ID NO: 1.

In some embodiments, this disclosure provides a KOD polymerase variant comprising: a Q at a position corresponding to L158 of SEQ ID NO: 1. In some embodiments, this disclosure provides a KOD polymerase variant comprising: a Q at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position L158 of SEQ ID NO: 1.

In some embodiments, this disclosure provides a KOD polymerase variant comprising: a R at a position corresponding to L158 of SEQ ID NO: 1. In some embodiments, this disclosure provides a KOD polymerase variant comprising: a R at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position L158 of SEQ ID NO: 1.

In some embodiments, this disclosure provides a KOD polymerase variant comprising: a W at a position corresponding to L158 of SEQ ID NO: 1. In some embodiments, this disclosure provides a KOD polymerase variant comprising: a W at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position L158 of SEQ ID NO: 1.

In some embodiments, this disclosure provides a KOD polymerase variant comprising: a Y at a position corresponding to L158 of SEQ ID NO: 1. In some embodiments, this disclosure provides a KOD polymerase variant comprising: a Y at position L158 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position L158 of SEQ ID NO: 1.

### M159

In some embodiments, a KOD polymerase variant comprises: an E, G, P, or R at a position corresponding to M159 of SEQ ID NO: 1. In some embodiments, a KOD polymerase variant comprises: an E, G, P, or R at position M159 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E, G, P, or R at position M159 of SEQ ID NO: 1.

In some embodiments, this disclosure provides a KOD polymerase variant comprising: an E at a position corresponding to M159 of SEQ ID NO: 1. In some embodiments, this disclosure provides a KOD polymerase variant comprising: an E at position M159 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position M159 of SEQ ID NO: 1.

In some embodiments, this disclosure provides a KOD polymerase variant comprising: a G at a position corresponding to M159 of SEQ ID NO: 1. In some embodiments, this disclosure provides a KOD polymerase variant comprising: a G at position M159 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position M159 of SEQ ID NO: 1.

In some embodiments, this disclosure provides a KOD polymerase variant comprising: a P at a position corresponding to M159 of SEQ ID NO: 1. In some embodiments, this disclosure provides a KOD polymerase variant comprising: a P at position M159 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position M159 of SEQ ID NO: 1.

In some embodiments, this disclosure provides a KOD polymerase variant comprising: a R at a position corresponding to M159 of SEQ ID NO: 1. In some embodiments, this disclosure provides a KOD polymerase variant comprising: a R at position M159 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position M159 of SEQ ID NO: 1.

### I160

In some embodiments, this disclosure provides a KOD polymerase variant comprising: a F at a position corresponding to I160 of SEQ ID NO: 1. In some embodiments, this disclosure provides a KOD polymerase variant comprising: a F at position I160 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I160 of SEQ ID NO: 1.

### S161

In some embodiments, a KOD polymerase variant comprises: a D, E, F, H, K, L, M, N, P, R, T, W, or Y at a position corresponding to S161 of SEQ ID NO: 1. In some embodiments, a KOD polymerase variant comprises: a D, E, F, H, K, L, M, N, P, R, T, W, or Y at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, H, K, L, M, N, P, R, T, W, or Y at position S161 of SEQ ID NO: 1.

In some embodiments, this disclosure provides a KOD polymerase variant comprising: a D at a position corresponding to S161 of SEQ ID NO: 1. In some embodiments, this disclosure provides a KOD polymerase variant comprising: a D at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position S161 of SEQ ID NO: 1.

In some embodiments, this disclosure provides a KOD polymerase variant comprising: an E at a position corresponding to S161 of SEQ ID NO: 1. In some embodiments, this disclosure provides a KOD polymerase variant comprising: an E at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position S161 of SEQ ID NO: 1.

In some embodiments, this disclosure provides a KOD polymerase variant comprising: a F at a position corresponding to S161 of SEQ ID NO: 1. In some embodiments, this disclosure provides a KOD polymerase variant comprising: a F at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position S161 of SEQ ID NO: 1.

In some embodiments, this disclosure provides a KOD polymerase variant comprising: a H at a position corresponding to S161 of SEQ ID NO: 1. In some embodiments, this disclosure provides a KOD polymerase variant comprising: a H at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position S161 of SEQ ID NO: 1.

In some embodiments, this disclosure provides a KOD polymerase variant comprising: a K at a position corresponding to S161 of SEQ ID NO: 1. In some embodiments, this disclosure provides a KOD polymerase variant comprising: a K at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position S161 of SEQ ID NO: 1.

In some embodiments, this disclosure provides a KOD polymerase variant comprising: a L at a position corresponding to S161 of SEQ ID NO: 1. In some embodiments, this disclosure provides a KOD polymerase variant comprising: a L at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position S161 of SEQ ID NO: 1.

In some embodiments, this disclosure provides a KOD polymerase variant comprising: a M at a position corresponding to S161 of SEQ ID NO: 1. In some embodiments, this disclosure provides a KOD polymerase variant comprising: a M at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position S161 of SEQ ID NO: 1.

In some embodiments, this disclosure provides a KOD polymerase variant comprising: a N at a position corresponding to S161 of SEQ ID NO: 1. In some embodiments, this disclosure provides a KOD polymerase variant comprising: a N at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position S161 of SEQ ID NO: 1.

In some embodiments, this disclosure provides a KOD polymerase variant comprising: a P at a position corresponding to S161 of SEQ ID NO: 1. In some embodiments, this disclosure provides a KOD polymerase variant comprising: a P at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position S161 of SEQ ID NO: 1.

In some embodiments, this disclosure provides a KOD polymerase variant comprising: a R at a position corresponding to S161 of SEQ ID NO: 1. In some embodiments, this disclosure provides a KOD polymerase variant comprising: a R at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position S161 of SEQ ID NO: 1.

In some embodiments, this disclosure provides a KOD polymerase variant comprising: a T at a position corresponding to S161 of SEQ ID NO: 1. In some embodiments, this disclosure provides a KOD polymerase variant comprising: a T at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position S161 of SEQ ID NO: 1.

In some embodiments, this disclosure provides a KOD polymerase variant comprising: a W at a position corresponding to S161 of SEQ ID NO: 1. In some embodiments, this disclosure provides a KOD polymerase variant comprising: a W at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position S161 of SEQ ID NO: 1.

In some embodiments, this disclosure provides a KOD polymerase variant comprising: a Y at a position corresponding to S161 of SEQ ID NO: 1. In some embodiments, this disclosure provides a KOD polymerase variant comprising: a Y at position S161 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position S161 of SEQ ID NO: 1.

### Y162

In some embodiments, this disclosure provides a KOD polymerase variant comprising: a P at a position corresponding to Y162 of SEQ ID NO: 1. In some embodiments, this disclosure provides a KOD polymerase variant comprising: a P at position Y162 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y162 of SEQ ID NO: 1.

### A168

In some embodiments, a KOD polymerase variant comprises: a C, E, F, G, N, or T at a position corresponding to A168 of SEQ ID NO: 1. In some embodiments, a KOD polymerase variant comprises: a C, E, F, G, N, or T at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, E, F, G, N, or T at position A168 of SEQ ID NO: 1.

In some embodiments, this disclosure provides a KOD polymerase variant comprising: a C at a position corresponding to A168 of SEQ ID NO: 1. In some embodiments, this disclosure provides a KOD polymerase variant comprising: a C at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position A168 of SEQ ID NO: 1.

In some embodiments, this disclosure provides a KOD polymerase variant comprising: an E at a position corresponding to A168 of SEQ ID NO: 1. In some embodiments, this disclosure provides a KOD polymerase variant comprising: an E at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A168 of SEQ ID NO: 1.

In some embodiments, this disclosure provides a KOD polymerase variant comprising: a F at a position corresponding to A168 of SEQ ID NO: 1. In some embodiments, this disclosure provides a KOD polymerase variant comprising: a F at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position A168 of SEQ ID NO: 1.

In some embodiments, this disclosure provides a KOD polymerase variant comprising: a G at a position corresponding to A168 of SEQ ID NO: 1. In some embodiments, this disclosure provides a KOD polymerase variant comprising: a G at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position A168 of SEQ ID NO: 1.

In some embodiments, this disclosure provides a KOD polymerase variant comprising: a N at a position corresponding to A168 of SEQ ID NO: 1. In some embodiments, this disclosure provides a KOD polymerase variant comprising: a N at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position A168 of SEQ ID NO: 1.

In some embodiments, this disclosure provides a KOD polymerase variant comprising: a T at a position corresponding to A168 of SEQ ID NO: 1. In some embodiments, this disclosure provides a KOD polymerase variant comprising: a T at position A168 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position A168 of SEQ ID NO: 1.

### V170

In some embodiments, a KOD polymerase variant comprises: an A, G, H, R, S, W, or Y at a position corresponding to V170 of SEQ ID NO: 1. In some embodiments, a KOD polymerase variant comprises: an A, G, H, R, S, W, or Y at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, G, H, R, S, W, or Y at position V170 of SEQ ID NO: 1.

In some embodiments, this disclosure provides a KOD polymerase variant comprising: an A at a position corresponding to V170 of SEQ ID NO: 1. In some embodiments, this disclosure provides a KOD polymerase variant comprising: an A at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position V170 of SEQ ID NO: 1.

In some embodiments, this disclosure provides a KOD polymerase variant comprising: a G at a position corresponding to V170 of SEQ ID NO: 1. In some embodiments, this disclosure provides a KOD polymerase variant comprising: a G at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position V170 of SEQ ID NO: 1.

In some embodiments, this disclosure provides a KOD polymerase variant comprising: a H at a position corresponding to V170 of SEQ ID NO: 1. In some embodiments, this disclosure provides a KOD polymerase variant comprising: a H at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position V170 of SEQ ID NO: 1.

In some embodiments, this disclosure provides a KOD polymerase variant comprising: a R at a position corresponding to V170 of SEQ ID NO: 1. In some embodiments, this disclosure provides a KOD polymerase variant comprising: a R at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position V170 of SEQ ID NO: 1.

In some embodiments, this disclosure provides a KOD polymerase variant comprising: a S at a position corresponding to V170 of SEQ ID NO: 1. In some embodiments, this disclosure provides a KOD polymerase variant comprising: a S at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position V170 of SEQ ID NO: 1.

In some embodiments, this disclosure provides a KOD polymerase variant comprising: a W at a position corresponding to V170 of SEQ ID NO: 1. In some embodiments, this disclosure provides a KOD polymerase variant comprising: a W at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position V170 of SEQ ID NO: 1.

In some embodiments, this disclosure provides a KOD polymerase variant comprising: a Y at a position corresponding to V170 of SEQ ID NO: 1. In some embodiments, this disclosure provides a KOD polymerase variant comprising: a Y at position V170 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position V170 of SEQ ID NO: 1.

### T172

In some embodiments, this disclosure provides a KOD polymerase variant comprising: a R at a position corresponding to T172 of SEQ ID NO: 1. In some embodiments, this disclosure provides a KOD polymerase variant comprising: a R at position T172 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position T172 of SEQ ID NO: 1.

### E187

In some embodiments, this disclosure provides a KOD polymerase variant comprising: a W at a position corresponding to E187 of SEQ ID NO: 1. In some embodiments, this disclosure provides a KOD polymerase variant comprising: a W at position E187 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position E187 of SEQ ID NO: 1.

In some embodiments, a KOD polymerase variant comprises: a F or Y at a position corresponding to I191 of SEQ ID NO: 1. In some embodiments, a KOD polymerase variant comprises: a F or Y at position I191 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F or Y at position I191 of SEQ ID NO: 1.

In some embodiments, this disclosure provides a KOD polymerase variant comprising: a F at a position corresponding to I191 of SEQ ID NO: 1. In some embodiments, this disclosure provides a KOD polymerase variant comprising: a F at position I191 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position I191 of SEQ ID NO: 1.

In some embodiments, this disclosure provides a KOD polymerase variant comprising: a Y at a position corresponding to I191 of SEQ ID NO: 1. In some embodiments, this disclosure provides a KOD polymerase variant comprising: a Y at position I191 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I191 of SEQ ID NO: 1.

### F194

In some embodiments, this disclosure provides a KOD polymerase variant comprising: a G at a position corresponding to F194 of SEQ ID NO: 1. In some embodiments, this disclosure provides a KOD polymerase variant comprising: a G at position F194 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position F194 of SEQ ID NO: 1.

### K199

In some embodiments, this disclosure provides a KOD polymerase variant comprising: a F at a position corresponding to K199 of SEQ ID NO: 1. In some embodiments, this disclosure provides a KOD polymerase variant comprising: a F at position K199 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position K199 of SEQ ID NO: 1.

### T208

In some embodiments, a KOD polymerase variant comprises: an E, F, H, I, K, N, Q, R, V, W, or Y at a position corresponding to T208 of SEQ ID NO: 1. In some embodiments, a KOD polymerase variant comprises: an E, F, H, I, K, N, Q, R, V, W, or Y at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E, F, H, I, K, N, Q, R, V, W, or Y at position T208 of SEQ ID NO: 1.

In some embodiments, this disclosure provides a KOD polymerase variant comprising: an E at a position corresponding to T208 of SEQ ID NO: 1. In some embodiments, this disclosure provides a KOD polymerase variant comprising: an E at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position T208 of SEQ ID NO: 1.

In some embodiments, this disclosure provides a KOD polymerase variant comprising: a F at a position corresponding to T208 of SEQ ID NO: 1. In some embodiments, this disclosure provides a KOD polymerase variant comprising: a F at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position T208 of SEQ ID NO: 1.

In some embodiments, this disclosure provides a KOD polymerase variant comprising: a H at a position corresponding to T208 of SEQ ID NO: 1. In some embodiments, this disclosure provides a KOD polymerase variant comprising: a H at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position T208 of SEQ ID NO: 1.

In some embodiments, this disclosure provides a KOD polymerase variant comprising: an I at a position corresponding to T208 of SEQ ID NO: 1. In some embodiments, this disclosure provides a KOD polymerase variant comprising: an I at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position T208 of SEQ ID NO: 1.

In some embodiments, this disclosure provides a KOD polymerase variant comprising: a K at a position corresponding to T208 of SEQ ID NO: 1. In some embodiments, this disclosure provides a KOD polymerase variant comprising: a K at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position T208 of SEQ ID NO: 1.

In some embodiments, this disclosure provides a KOD polymerase variant comprising: a N at a position corresponding to T208 of SEQ ID NO: 1. In some embodiments, this disclosure provides a KOD polymerase variant comprising: a N at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position T208 of SEQ ID NO: 1.

In some embodiments, this disclosure provides a KOD polymerase variant comprising: a Q at a position corresponding to T208 of SEQ ID NO: 1. In some embodiments, this disclosure provides a KOD polymerase variant comprising: a Q at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position T208 of SEQ ID NO: 1.

In some embodiments, this disclosure provides a KOD polymerase variant comprising: a R at a position corresponding to T208 of SEQ ID NO: 1. In some embodiments, this disclosure provides a KOD polymerase variant comprising: a R at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position T208 of SEQ ID NO: 1.

In some embodiments, this disclosure provides a KOD polymerase variant comprising: a V at a position corresponding to T208 of SEQ ID NO: 1. In some embodiments, this disclosure provides a KOD polymerase variant comprising: a V at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position T208 of SEQ ID NO: 1.

In some embodiments, this disclosure provides a KOD polymerase variant comprising: a W at a position corresponding to T208 of SEQ ID NO: 1. In some embodiments, this disclosure provides a KOD polymerase variant comprising: a W at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position T208 of SEQ ID NO: 1.

In some embodiments, this disclosure provides a KOD polymerase variant comprising: a Y at a position corresponding to T208 of SEQ ID NO: 1. In some embodiments, this disclosure provides a KOD polymerase variant comprising: a Y at position T208 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position T208 of SEQ ID NO: 1.

### Y209

In some embodiments, a KOD polymerase variant comprises: an A, C, E, G, H, I, L, M, N, P, S, T, V, or W at a position corresponding to Y209 of SEQ ID NO: 1. In some embodiments, a KOD polymerase variant comprises: an A, C, E, G, H, I, L, M, N, P, S, T, V, or W at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, E, G, H, I, L, M, N, P, S, T, V, or W at position Y209 of SEQ ID NO: 1.

In some embodiments, this disclosure provides a KOD polymerase variant comprising: an A at a position corresponding to Y209 of SEQ ID NO: 1. In some embodiments, this disclosure provides a KOD polymerase variant comprising: an A at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position Y209 of SEQ ID NO: 1.

In some embodiments, this disclosure provides a KOD polymerase variant comprising: a C at a position corresponding to Y209 of SEQ ID NO: 1. In some embodiments, this disclosure provides a KOD polymerase variant comprising: a C at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position Y209 of SEQ ID NO: 1.

In some embodiments, this disclosure provides a KOD polymerase variant comprising: an E at a position corresponding to Y209 of SEQ ID NO: 1. In some embodiments, this disclosure provides a KOD polymerase variant comprising: an E at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y209 of SEQ ID NO: 1.

In some embodiments, this disclosure provides a KOD polymerase variant comprising: a G at a position corresponding to Y209 of SEQ ID NO: 1. In some embodiments, this disclosure provides a KOD polymerase variant comprising: a G at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position Y209 of SEQ ID NO: 1.

In some embodiments, this disclosure provides a KOD polymerase variant comprising: a H at a position corresponding to Y209 of SEQ ID NO: 1. In some embodiments, this disclosure provides a KOD polymerase variant comprising: a H at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position Y209 of SEQ ID NO: 1.

In some embodiments, this disclosure provides a KOD polymerase variant comprising: an I at a position corresponding to Y209 of SEQ ID NO: 1. In some embodiments, this disclosure provides a KOD polymerase variant comprising: an I at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position Y209 of SEQ ID NO: 1.

In some embodiments, this disclosure provides a KOD polymerase variant comprising: a L at a position corresponding to Y209 of SEQ ID NO: 1. In some embodiments, this disclosure provides a KOD polymerase variant comprising: a L at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position Y209 of SEQ ID NO: 1.

In some embodiments, this disclosure provides a KOD polymerase variant comprising: a M at a position corresponding to Y209 of SEQ ID NO: 1. In some embodiments, this disclosure provides a KOD polymerase variant comprising: a M at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position Y209 of SEQ ID NO: 1.

In some embodiments, this disclosure provides a KOD polymerase variant comprising: a N at a position corresponding to Y209 of SEQ ID NO: 1. In some embodiments, this disclosure provides a KOD polymerase variant comprising: a N at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position Y209 of SEQ ID NO: 1.

In some embodiments, this disclosure provides a KOD polymerase variant comprising: a P at a position corresponding to Y209 of SEQ ID NO: 1. In some embodiments, this disclosure provides a KOD polymerase variant comprising: a P at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y209 of SEQ ID NO: 1.

In some embodiments, this disclosure provides a KOD polymerase variant comprising: a S at a position corresponding to Y209 of SEQ ID NO: 1. In some embodiments, this disclosure provides a KOD polymerase variant comprising: a S at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position Y209 of SEQ ID NO: 1.

In some embodiments, this disclosure provides a KOD polymerase variant comprising: a T at a position corresponding to Y209 of SEQ ID NO: 1. In some embodiments, this disclosure provides a KOD polymerase variant comprising: a T at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position Y209 of SEQ ID NO: 1.

In some embodiments, this disclosure provides a KOD polymerase variant comprising: a V at a position corresponding to Y209 of SEQ ID NO: 1. In some embodiments, this disclosure provides a KOD polymerase variant comprising: a V at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position Y209 of SEQ ID NO: 1.

In some embodiments, this disclosure provides a KOD polymerase variant comprising: a W at a position corresponding to Y209 of SEQ ID NO: 1. In some embodiments, this disclosure provides a KOD polymerase variant comprising: a W at position Y209 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position Y209 of SEQ ID NO: 1.

### N210

In some embodiments, a KOD polymerase variant comprises: a C, D, E, G, M, P, Q, S, or T at a position corresponding to N210 of SEQ ID NO: 1. In some embodiments, a KOD polymerase variant comprises: a C, D, E, G, M, P, Q, S, or T at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, G, M, P, Q, S, or T at position N210 of SEQ ID NO: 1.

In some embodiments, this disclosure provides a KOD polymerase variant comprising: a C at a position corresponding to N210 of SEQ ID NO: 1. In some embodiments, this disclosure provides a KOD polymerase variant comprising: a C at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position N210 of SEQ ID NO: 1.

In some embodiments, this disclosure provides a KOD polymerase variant comprising: a D at a position corresponding to N210 of SEQ ID NO: 1. In some embodiments, this disclosure provides a KOD polymerase variant comprising: a D at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position N210 of SEQ ID NO: 1.

In some embodiments, this disclosure provides a KOD polymerase variant comprising: an E at a position corresponding to N210 of SEQ ID NO: 1. In some embodiments, this disclosure provides a KOD polymerase variant comprising: an E at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position N210 of SEQ ID NO: 1.

In some embodiments, this disclosure provides a KOD polymerase variant comprising: a G at a position corresponding to N210 of SEQ ID NO: 1. In some embodiments, this disclosure provides a KOD polymerase variant comprising: a G at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position N210 of SEQ ID NO: 1.

In some embodiments, this disclosure provides a KOD polymerase variant comprising: a M at a position corresponding to N210 of SEQ ID NO: 1. In some embodiments, this disclosure provides a KOD polymerase variant comprising: a M at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position N210 of SEQ ID NO: 1.

In some embodiments, this disclosure provides a KOD polymerase variant comprising: a P at a position corresponding to N210 of SEQ ID NO: 1. In some embodiments, this disclosure provides a KOD polymerase variant comprising: a P at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position N210 of SEQ ID NO: 1.

In some embodiments, this disclosure provides a KOD polymerase variant comprising: a Q at a position corresponding to N210 of SEQ ID NO: 1. In some embodiments, this disclosure provides a KOD polymerase variant comprising: a Q at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position N210 of SEQ ID NO: 1.

In some embodiments, this disclosure provides a KOD polymerase variant comprising: a S at a position corresponding to N210 of SEQ ID NO: 1. In some embodiments, this disclosure provides a KOD polymerase variant comprising: a S at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position N210 of SEQ ID NO: 1.

In some embodiments, this disclosure provides a KOD polymerase variant comprising: a T at a position corresponding to N210 of SEQ ID NO: 1. In some embodiments, this disclosure provides a KOD polymerase variant comprising: a T at position N210 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position N210 of SEQ ID NO: 1.

### G211

In some embodiments, a KOD polymerase variant comprises: a C, D, E, F, H, I, K, L, M, N, P, Q, R, S, T, V, W, or Y at a position corresponding to G211 of SEQ ID NO: 1. In some embodiments, a KOD polymerase variant comprises: a C, D, E, F, H, I, K, L, M, N, P, Q, R, S, T, V, W, or Y at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, D, E, F, H, I, K, L, M, N, P, Q, R, S, T, V, W, or Y at position G211 of SEQ ID NO: 1.

In some embodiments, this disclosure provides a KOD polymerase variant comprising: a C at a position corresponding to G211 of SEQ ID NO: 1. In some embodiments, this disclosure provides a KOD polymerase variant comprising: a C at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position G211 of SEQ ID NO: 1.

In some embodiments, this disclosure provides a KOD polymerase variant comprising: a D at a position corresponding to G211 of SEQ ID NO: 1. In some embodiments, this disclosure provides a KOD polymerase variant comprising: a D at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position G211 of SEQ ID NO: 1.

In some embodiments, this disclosure provides a KOD polymerase variant comprising: an E at a position corresponding to G211 of SEQ ID NO: 1. In some embodiments, this disclosure provides a KOD polymerase variant comprising: an E at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position G211 of SEQ ID NO: 1.

In some embodiments, this disclosure provides a KOD polymerase variant comprising: a F at a position corresponding to G211 of SEQ ID NO: 1. In some embodiments, this disclosure provides a KOD polymerase variant comprising: a F at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position G211 of SEQ ID NO: 1.

In some embodiments, this disclosure provides a KOD polymerase variant comprising: a H at a position corresponding to G211 of SEQ ID NO: 1. In some embodiments, this disclosure provides a KOD polymerase variant comprising: a H at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position G211 of SEQ ID NO: 1.

In some embodiments, this disclosure provides a KOD polymerase variant comprising: an I at a position corresponding to G211 of SEQ ID NO: 1. In some embodiments, this disclosure provides a KOD polymerase variant comprising: an I at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position G211 of SEQ ID NO: 1.

In some embodiments, this disclosure provides a KOD polymerase variant comprising: a K at a position corresponding to G211 of SEQ ID NO: 1. In some embodiments, this disclosure provides a KOD polymerase variant comprising: a K at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position G211 of SEQ ID NO: 1.

In some embodiments, this disclosure provides a KOD polymerase variant comprising: a L at a position corresponding to G211 of SEQ ID NO: 1. In some embodiments, this disclosure provides a KOD polymerase variant comprising: a L at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position G211 of SEQ ID NO: 1.

In some embodiments, this disclosure provides a KOD polymerase variant comprising: a M at a position corresponding to G211 of SEQ ID NO: 1. In some embodiments, this disclosure provides a KOD polymerase variant comprising: a M at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position G211 of SEQ ID NO: 1.

In some embodiments, this disclosure provides a KOD polymerase variant comprising: a N at a position corresponding to G211 of SEQ ID NO: 1. In some embodiments, this disclosure provides a KOD polymerase variant comprising: a N at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position G211 of SEQ ID NO: 1.

In some embodiments, this disclosure provides a KOD polymerase variant comprising: a P at a position corresponding to G211 of SEQ ID NO: 1. In some embodiments, this disclosure provides a KOD polymerase variant comprising: a P at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position G211 of SEQ ID NO: 1.

In some embodiments, this disclosure provides a KOD polymerase variant comprising: a Q at a position corresponding to G211 of SEQ ID NO: 1. In some embodiments, this disclosure provides a KOD polymerase variant comprising: a Q at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position G211 of SEQ ID NO: 1.

In some embodiments, this disclosure provides a KOD polymerase variant comprising: a R at a position corresponding to G211 of SEQ ID NO: 1. In some embodiments, this disclosure provides a KOD polymerase variant comprising: a R at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position G211 of SEQ ID NO: 1.

In some embodiments, this disclosure provides a KOD polymerase variant comprising: a S at a position corresponding to G211 of SEQ ID NO: 1. In some embodiments, this disclosure provides a KOD polymerase variant comprising: a S at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position G211 of SEQ ID NO: 1.

In some embodiments, this disclosure provides a KOD polymerase variant comprising: a T at a position corresponding to G211 of SEQ ID NO: 1. In some embodiments, this disclosure provides a KOD polymerase variant comprising: a T at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position G211 of SEQ ID NO: 1.

In some embodiments, this disclosure provides a KOD polymerase variant comprising: a V at a position corresponding to G211 of SEQ ID NO: 1. In some embodiments, this disclosure provides a KOD polymerase variant comprising: a V at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position G211 of SEQ ID NO: 1.

In some embodiments, this disclosure provides a KOD polymerase variant comprising: a W at a position corresponding to G211 of SEQ ID NO: 1. In some embodiments, this disclosure provides a KOD polymerase variant comprising: a W at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position G211 of SEQ ID NO: 1.

In some embodiments, this disclosure provides a KOD polymerase variant comprising: a Y at a position corresponding to G211 of SEQ ID NO: 1. In some embodiments, this disclosure provides a KOD polymerase variant comprising: a Y at position G211 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position G211 of SEQ ID NO: 1.

### D212

In some embodiments, a KOD polymerase variant comprises: a F, I, K, L, M, P, Q, R, V, W, or Y at a position corresponding to D212 of SEQ ID NO: 1. In some embodiments, a KOD polymerase variant comprises: a F, I, K, L, M, P, Q, R, V, W, or Y at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F, I, K, L, M, P, Q, R, V, W, or Y at position D212 of SEQ ID NO: 1.

In some embodiments, this disclosure provides a KOD polymerase variant comprising: a F at a position corresponding to D212 of SEQ ID NO: 1. In some embodiments, this disclosure provides a KOD polymerase variant comprising: a F at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position D212 of SEQ ID NO: 1.

In some embodiments, this disclosure provides a KOD polymerase variant comprising: an I at a position corresponding to D212 of SEQ ID NO: 1. In some embodiments, this disclosure provides a KOD polymerase variant comprising: an I at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D212 of SEQ ID NO: 1.

In some embodiments, this disclosure provides a KOD polymerase variant comprising: a K at a position corresponding to D212 of SEQ ID NO: 1. In some embodiments, this disclosure provides a KOD polymerase variant comprising: a K at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D212 of SEQ ID NO: 1.

In some embodiments, this disclosure provides a KOD polymerase variant comprising: a L at a position corresponding to D212 of SEQ ID NO: 1. In some embodiments, this disclosure provides a KOD polymerase variant comprising: a L at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D212 of SEQ ID NO: 1.

In some embodiments, this disclosure provides a KOD polymerase variant comprising: a M at a position corresponding to D212 of SEQ ID NO: 1. In some embodiments, this disclosure provides a KOD polymerase variant comprising: a M at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D212 of SEQ ID NO: 1.

In some embodiments, this disclosure provides a KOD polymerase variant comprising: a P at a position corresponding to D212 of SEQ ID NO: 1. In some embodiments, this disclosure provides a KOD polymerase variant comprising: a P at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D212 of SEQ ID NO: 1.

In some embodiments, this disclosure provides a KOD polymerase variant comprising: a Q at a position corresponding to D212 of SEQ ID NO: 1. In some embodiments, this disclosure provides a KOD polymerase variant comprising: a Q at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D212 of SEQ ID NO: 1.

In some embodiments, this disclosure provides a KOD polymerase variant comprising: a R at a position corresponding to D212 of SEQ ID NO: 1. In some embodiments, this disclosure provides a KOD polymerase variant comprising: a R at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position D212 of SEQ ID NO: 1.

In some embodiments, this disclosure provides a KOD polymerase variant comprising: a V at a position corresponding to D212 of SEQ ID NO: 1. In some embodiments, this disclosure provides a KOD polymerase variant comprising: a V at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D212 of SEQ ID NO: 1.

In some embodiments, this disclosure provides a KOD polymerase variant comprising: a W at a position corresponding to D212 of SEQ ID NO: 1. In some embodiments, this disclosure provides a KOD polymerase variant comprising: a W at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position D212 of SEQ ID NO: 1.

In some embodiments, this disclosure provides a KOD polymerase variant comprising: a Y at a position corresponding to D212 of SEQ ID NO: 1. In some embodiments, this disclosure provides a KOD polymerase variant comprising: a Y at position D212 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D212 of SEQ ID NO: 1.

### N213

In some embodiments, a KOD polymerase variant comprises: a D or E at a position corresponding to N213 of SEQ ID NO: 1. In some embodiments, a KOD polymerase variant comprises: a D or E at position N213 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or E at position N213 of SEQ ID NO: 1.

In some embodiments, this disclosure provides a KOD polymerase variant comprising: a D at a position corresponding to N213 of SEQ ID NO: 1. In some embodiments, this disclosure provides a KOD polymerase variant comprising: a D at position N213 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position N213 of SEQ ID NO: 1.

In some embodiments, this disclosure provides a KOD polymerase variant comprising: an E at a position corresponding to N213 of SEQ ID NO: 1. In some embodiments, this disclosure provides a KOD polymerase variant comprising: an E at position N213 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position N213 of SEQ ID NO: 1.

### F214

In some embodiments, a KOD polymerase variant comprises: an A, C, D, E, G, H, I, K, L, M, N, P, Q, R, S, T, V, or W at a position corresponding to F214 of SEQ ID NO: 1. In some embodiments, a KOD polymerase variant comprises: an A, C, D, E, G, H, I, K, L, M, N, P, Q, R, S, T, V, or W at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, G, H, I, K, L, M, N, P, Q, R, S, T, V, or W at position F214 of SEQ ID NO: 1.

In some embodiments, this disclosure provides a KOD polymerase variant comprising: an A at a position corresponding to F214 of SEQ ID NO: 1. In some embodiments, this disclosure provides a KOD polymerase variant comprising: an A at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position F214 of SEQ ID NO: 1.

In some embodiments, this disclosure provides a KOD polymerase variant comprising: a C at a position corresponding to F214 of SEQ ID NO: 1. In some embodiments, this disclosure provides a KOD polymerase variant comprising: a C at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position F214 of SEQ ID NO: 1.

In some embodiments, this disclosure provides a KOD polymerase variant comprising: a D at a position corresponding to F214 of SEQ ID NO: 1. In some embodiments, this disclosure provides a KOD polymerase variant comprising: a D at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position F214 of SEQ ID NO: 1.

In some embodiments, this disclosure provides a KOD polymerase variant comprising: an E at a position corresponding to F214 of SEQ ID NO: 1. In some embodiments, this disclosure provides a KOD polymerase variant comprising: an E at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position F214 of SEQ ID NO: 1.

In some embodiments, this disclosure provides a KOD polymerase variant comprising: a G at a position corresponding to F214 of SEQ ID NO: 1. In some embodiments, this disclosure provides a KOD polymerase variant comprising: a G at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position F214 of SEQ ID NO: 1.

In some embodiments, this disclosure provides a KOD polymerase variant comprising: a H at a position corresponding to F214 of SEQ ID NO: 1. In some embodiments, this disclosure provides a KOD polymerase variant comprising: a H at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position F214 of SEQ ID NO: 1.

In some embodiments, this disclosure provides a KOD polymerase variant comprising: an I at a position corresponding to F214 of SEQ ID NO: 1. In some embodiments, this disclosure provides a KOD polymerase variant comprising: an I at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position F214 of SEQ ID NO: 1.

In some embodiments, this disclosure provides a KOD polymerase variant comprising: a K at a position corresponding to F214 of SEQ ID NO: 1. In some embodiments, this disclosure provides a KOD polymerase variant comprising: a K at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position F214 of SEQ ID NO: 1.

In some embodiments, this disclosure provides a KOD polymerase variant comprising: a L at a position corresponding to F214 of SEQ ID NO: 1. In some embodiments, this disclosure provides a KOD polymerase variant comprising: a L at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position F214 of SEQ ID NO: 1.

In some embodiments, this disclosure provides a KOD polymerase variant comprising: a M at a position corresponding to F214 of SEQ ID NO: 1. In some embodiments, this disclosure provides a KOD polymerase variant comprising: a M at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position F214 of SEQ ID NO: 1.

In some embodiments, this disclosure provides a KOD polymerase variant comprising: a N at a position corresponding to F214 of SEQ ID NO: 1. In some embodiments, this disclosure provides a KOD polymerase variant comprising: a N at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position F214 of SEQ ID NO: 1.

In some embodiments, this disclosure provides a KOD polymerase variant comprising: a P at a position corresponding to F214 of SEQ ID NO: 1. In some embodiments, this disclosure provides a KOD polymerase variant comprising: a P at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position F214 of SEQ ID NO: 1.

In some embodiments, this disclosure provides a KOD polymerase variant comprising: a Q at a position corresponding to F214 of SEQ ID NO: 1. In some embodiments, this disclosure provides a KOD polymerase variant comprising: a Q at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position F214 of SEQ ID NO: 1.

In some embodiments, this disclosure provides a KOD polymerase variant comprising: a R at a position corresponding to F214 of SEQ ID NO: 1. In some embodiments, this disclosure provides a KOD polymerase variant comprising: a R at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position F214 of SEQ ID NO: 1.

In some embodiments, this disclosure provides a KOD polymerase variant comprising: a S at a position corresponding to F214 of SEQ ID NO: 1. In some embodiments, this disclosure provides a KOD polymerase variant comprising: a S at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position F214 of SEQ ID NO: 1.

In some embodiments, this disclosure provides a KOD polymerase variant comprising: a T at a position corresponding to F214 of SEQ ID NO: 1. In some embodiments, this disclosure provides a KOD polymerase variant comprising: a T at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position F214 of SEQ ID NO: 1.

In some embodiments, this disclosure provides a KOD polymerase variant comprising: a V at a position corresponding to F214 of SEQ ID NO: 1. In some embodiments, this disclosure provides a KOD polymerase variant comprising: a V at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position F214 of SEQ ID NO: 1.

In some embodiments, this disclosure provides a KOD polymerase variant comprising: a W at a position corresponding to F214 of SEQ ID NO: 1. In some embodiments, this disclosure provides a KOD polymerase variant comprising: a W at position F214 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position F214 of SEQ ID NO: 1.

### D215

In some embodiments, a KOD polymerase variant comprises: an A, C, F, G, H, I, K, L, M, N, P, Q, R, S, T, V, W, or Y at a position corresponding to D215 of SEQ ID NO: 1. In some embodiments, a KOD polymerase variant comprises: an A, C, F, G, H, I, K, L, M, N, P, Q, R, S, T, V, W, or Y at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, G, H, I, K, L, M, N, P, Q, R, S, T, V, W, or Y at position D215 of SEQ ID NO: 1.

In some embodiments, this disclosure provides a KOD polymerase variant comprising: an A at a position corresponding to D215 of SEQ ID NO: 1. In some embodiments, this disclosure provides a KOD polymerase variant comprising: an A at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position D215 of SEQ ID NO: 1.

In some embodiments, this disclosure provides a KOD polymerase variant comprising: a C at a position corresponding to D215 of SEQ ID NO: 1. In some embodiments, this disclosure provides a KOD polymerase variant comprising: a C at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position D215 of SEQ ID NO: 1.

In some embodiments, this disclosure provides a KOD polymerase variant comprising: a F at a position corresponding to D215 of SEQ ID NO: 1. In some embodiments, this disclosure provides a KOD polymerase variant comprising: a F at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position D215 of SEQ ID NO: 1.

In some embodiments, this disclosure provides a KOD polymerase variant comprising: a G at a position corresponding to D215 of SEQ ID NO: 1. In some embodiments, this disclosure provides a KOD polymerase variant comprising: a G at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position D215 of SEQ ID NO: 1.

In some embodiments, this disclosure provides a KOD polymerase variant comprising: a H at a position corresponding to D215 of SEQ ID NO: 1. In some embodiments, this disclosure provides a KOD polymerase variant comprising: a H at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position D215 of SEQ ID NO: 1.

In some embodiments, this disclosure provides a KOD polymerase variant comprising: an I at a position corresponding to D215 of SEQ ID NO: 1. In some embodiments, this disclosure provides a KOD polymerase variant comprising: an I at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D215 of SEQ ID NO: 1.

In some embodiments, this disclosure provides a KOD polymerase variant comprising: a K at a position corresponding to D215 of SEQ ID NO: 1. In some embodiments, this disclosure provides a KOD polymerase variant comprising: a K at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D215 of SEQ ID NO: 1.

In some embodiments, this disclosure provides a KOD polymerase variant comprising: a L at a position corresponding to D215 of SEQ ID NO: 1. In some embodiments, this disclosure provides a KOD polymerase variant comprising: a L at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D215 of SEQ ID NO: 1.

In some embodiments, this disclosure provides a KOD polymerase variant comprising: a M at a position corresponding to D215 of SEQ ID NO: 1. In some embodiments, this disclosure provides a KOD polymerase variant comprising: a M at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D215 of SEQ ID NO: 1.

In some embodiments, this disclosure provides a KOD polymerase variant comprising: a N at a position corresponding to D215 of SEQ ID NO: 1. In some embodiments, this disclosure provides a KOD polymerase variant comprising: a N at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position D215 of SEQ ID NO: 1.

In some embodiments, this disclosure provides a KOD polymerase variant comprising: a P at a position corresponding to D215 of SEQ ID NO: 1. In some embodiments, this disclosure provides a KOD polymerase variant comprising: a P at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D215 of SEQ ID NO: 1.

In some embodiments, this disclosure provides a KOD polymerase variant comprising: a Q at a position corresponding to D215 of SEQ ID NO: 1. In some embodiments, this disclosure provides a KOD polymerase variant comprising: a Q at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D215 of SEQ ID NO: 1.

In some embodiments, this disclosure provides a KOD polymerase variant comprising: a R at a position corresponding to D215 of SEQ ID NO: 1. In some embodiments, this disclosure provides a KOD polymerase variant comprising: a R at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position D215 of SEQ ID NO: 1.

In some embodiments, this disclosure provides a KOD polymerase variant comprising: a S at a position corresponding to D215 of SEQ ID NO: 1. In some embodiments, this disclosure provides a KOD polymerase variant comprising: a S at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position D215 of SEQ ID NO: 1.

In some embodiments, this disclosure provides a KOD polymerase variant comprising: a T at a position corresponding to D215 of SEQ ID NO: 1. In some embodiments, this disclosure provides a KOD polymerase variant comprising: a T at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D215 of SEQ ID NO: 1.

In some embodiments, this disclosure provides a KOD polymerase variant comprising: a V at a position corresponding to D215 of SEQ ID NO: 1. In some embodiments, this disclosure provides a KOD polymerase variant comprising: a V at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D215 of SEQ ID NO: 1.

In some embodiments, this disclosure provides a KOD polymerase variant comprising: a W at a position corresponding to D215 of SEQ ID NO: 1. In some embodiments, this disclosure provides a KOD polymerase variant comprising: a W at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position D215 of SEQ ID NO: 1.

In some embodiments, this disclosure provides a KOD polymerase variant comprising: a Y at a position corresponding to D215 of SEQ ID NO: 1. In some embodiments, this disclosure provides a KOD polymerase variant comprising: a Y at position D215 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D215 of SEQ ID NO: 1.

### F216

In some embodiments, a KOD polymerase variant comprises: an A or D at a position corresponding to F216 of SEQ ID NO: 1. In some embodiments, a KOD polymerase variant comprises: an A or D at position F216 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A or D at position F216 of SEQ ID NO: 1.

In some embodiments, this disclosure provides a KOD polymerase variant comprising: an A at a position corresponding to F216 of SEQ ID NO: 1. In some embodiments, this disclosure provides a KOD polymerase variant comprising: an A at position F216 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position F216 of SEQ ID NO: 1.

In some embodiments, this disclosure provides a KOD polymerase variant comprising: a D at a position corresponding to F216 of SEQ ID NO: 1. In some embodiments, this disclosure provides a KOD polymerase variant comprising: a D at position F216 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position F216 of SEQ ID NO: 1.

### Y218

In some embodiments, a KOD polymerase variant comprises: an A, D, E, G, L, P, S, T, or W at a position corresponding to Y218 of SEQ ID NO: 1. In some embodiments, a KOD polymerase variant comprises: an A, D, E, G, L, P, S, T, or W at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, G, L, P, S, T, or W at position Y218 of SEQ ID NO: 1.

In some embodiments, this disclosure provides a KOD polymerase variant comprising: an A at a position corresponding to Y218 of SEQ ID NO: 1. In some embodiments, this disclosure provides a KOD polymerase variant comprising: an A at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position Y218 of SEQ ID NO: 1.

In some embodiments, this disclosure provides a KOD polymerase variant comprising: a D at a position corresponding to Y218 of SEQ ID NO: 1. In some embodiments, this disclosure provides a KOD polymerase variant comprising: a D at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y218 of SEQ ID NO: 1.

In some embodiments, this disclosure provides a KOD polymerase variant comprising: an E at a position corresponding to Y218 of SEQ ID NO: 1. In some embodiments, this disclosure provides a KOD polymerase variant comprising: an E at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y218 of SEQ ID NO: 1.

In some embodiments, this disclosure provides a KOD polymerase variant comprising: a G at a position corresponding to Y218 of SEQ ID NO: 1. In some embodiments, this disclosure provides a KOD polymerase variant comprising: a G at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position Y218 of SEQ ID NO: 1.

In some embodiments, this disclosure provides a KOD polymerase variant comprising: a L at a position corresponding to Y218 of SEQ ID NO: 1. In some embodiments, this disclosure provides a KOD polymerase variant comprising: a L at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position Y218 of SEQ ID NO: 1.

In some embodiments, this disclosure provides a KOD polymerase variant comprising: a P at a position corresponding to Y218 of SEQ ID NO: 1. In some embodiments, this disclosure provides a KOD polymerase variant comprising: a P at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y218 of SEQ ID NO: 1.

In some embodiments, this disclosure provides a KOD polymerase variant comprising: a S at a position corresponding to Y218 of SEQ ID NO: 1. In some embodiments, this disclosure provides a KOD polymerase variant comprising: a S at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position Y218 of SEQ ID NO: 1.

In some embodiments, this disclosure provides a KOD polymerase variant comprising: a T at a position corresponding to Y218 of SEQ ID NO: 1. In some embodiments, this disclosure provides a KOD polymerase variant comprising: a T at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position Y218 of SEQ ID NO: 1.

In some embodiments, this disclosure provides a KOD polymerase variant comprising: a W at a position corresponding to Y218 of SEQ ID NO: 1. In some embodiments, this disclosure provides a KOD polymerase variant comprising: a W at position Y218 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position Y218 of SEQ ID NO: 1.

### L219

In some embodiments, a KOD polymerase variant comprises: a P, R, or V at a position corresponding to L219 of SEQ ID NO: 1. In some embodiments, a KOD polymerase variant comprises: a P, R, or V at position L219 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P, R, or V at position L219 of SEQ ID NO: 1.

In some embodiments, this disclosure provides a KOD polymerase variant comprising: a P at a position corresponding to L219 of SEQ ID NO: 1. In some embodiments, this disclosure provides a KOD polymerase variant comprising: a P at position L219 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position L219 of SEQ ID NO: 1.

In some embodiments, this disclosure provides a KOD polymerase variant comprising: a R at a position corresponding to L219 of SEQ ID NO: 1. In some embodiments, this disclosure provides a KOD polymerase variant comprising: a R at position L219 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position L219 of SEQ ID NO: 1.

In some embodiments, this disclosure provides a KOD polymerase variant comprising: a V at a position corresponding to L219 of SEQ ID NO: 1. In some embodiments, this disclosure provides a KOD polymerase variant comprising: a V at position L219 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position L219 of SEQ ID NO: 1.

### K220

In some embodiments, a KOD polymerase variant comprises: an I or V at a position corresponding to K220 of SEQ ID NO: 1. In some embodiments, a KOD polymerase variant comprises: an I or V at position K220 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I or V at position K220 of SEQ ID NO: 1.

In some embodiments, this disclosure provides a KOD polymerase variant comprising: an I at a position corresponding to K220 of SEQ ID NO: 1. In some embodiments, this disclosure provides a KOD polymerase variant comprising: an I at position K220 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position K220 of SEQ ID NO: 1.

In some embodiments, this disclosure provides a KOD polymerase variant comprising: a V at a position corresponding to K220 of SEQ ID NO: 1. In some embodiments, this disclosure provides a KOD polymerase variant comprising: a V at position K220 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position K220 of SEQ ID NO: 1.

### C223

In some embodiments, a KOD polymerase variant comprises: an A, E, or P at a position corresponding to C223 of SEQ ID NO: 1. In some embodiments, a KOD polymerase variant comprises: an A, E, or P at position C223 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, E, or P at position C223 of SEQ ID NO: 1.

In some embodiments, this disclosure provides a KOD polymerase variant comprising: an A at a position corresponding to C223 of SEQ ID NO: 1. In some embodiments, this disclosure provides a KOD polymerase variant comprising: an A at position C223 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position C223 of SEQ ID NO: 1.

In some embodiments, this disclosure provides a KOD polymerase variant comprising: an E at a position corresponding to C223 of SEQ ID NO: 1. In some embodiments, this disclosure provides a KOD polymerase variant comprising: an E at position C223 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position C223 of SEQ ID NO: 1.

In some embodiments, this disclosure provides a KOD polymerase variant comprising: a P at a position corresponding to C223 of SEQ ID NO: 1. In some embodiments, this disclosure provides a KOD polymerase variant comprising: a P at position C223 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position C223 of SEQ ID NO: 1.

### K240

In some embodiments, a KOD polymerase variant comprises: a W or Y at a position corresponding to K240 of SEQ ID NO: 1. In some embodiments, a KOD polymerase variant comprises: a W or Y at position K240 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W or Y at position K240 of SEQ ID NO: 1.

In some embodiments, this disclosure provides a KOD polymerase variant comprising: a W at a position corresponding to K240 of SEQ ID NO: 1. In some embodiments, this disclosure provides a KOD polymerase variant comprising: a W at position K240 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position K240 of SEQ ID NO: 1.

In some embodiments, this disclosure provides a KOD polymerase variant comprising: a Y at a position corresponding to K240 of SEQ ID NO: 1. In some embodiments, this disclosure provides a KOD polymerase variant comprising: a Y at position K240 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position K240 of SEQ ID NO: 1.

### A249

In some embodiments, this disclosure provides a KOD polymerase variant comprising: a D at a position corresponding to A249 of SEQ ID NO: 1. In some embodiments, this disclosure provides a KOD polymerase variant comprising: a D at position A249 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A249 of SEQ ID NO: 1.

### V250

In some embodiments, this disclosure provides a KOD polymerase variant comprising: a L at a position corresponding to V250 of SEQ ID NO: 1. In some embodiments, this disclosure provides a KOD polymerase variant comprising: a L at position V250 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position V250 of SEQ ID NO: 1.

### E251

In some embodiments, a KOD polymerase variant comprises: a F, G, N, or R at a position corresponding to E251 of SEQ ID NO: 1. In some embodiments, a KOD polymerase variant comprises: a F, G, N, or R at position E251 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F, G, N, or R at position E251 of SEQ ID NO: 1.

In some embodiments, this disclosure provides a KOD polymerase variant comprising: a F at a position corresponding to E251 of SEQ ID NO: 1. In some embodiments, this disclosure provides a KOD polymerase variant comprising: a F at position E251 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position E251 of SEQ ID NO: 1.

In some embodiments, this disclosure provides a KOD polymerase variant comprising: a G at a position corresponding to E251 of SEQ ID NO: 1. In some embodiments, this disclosure provides a KOD polymerase variant comprising: a G at position E251 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position E251 of SEQ ID NO: 1.

In some embodiments, this disclosure provides a KOD polymerase variant comprising: a N at a position corresponding to E251 of SEQ ID NO: 1. In some embodiments, this disclosure provides a KOD polymerase variant comprising: a N at position E251 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position E251 of SEQ ID NO: 1.

In some embodiments, this disclosure provides a KOD polymerase variant comprising: a R at a position corresponding to E251 of SEQ ID NO: 1. In some embodiments, this disclosure provides a KOD polymerase variant comprising: a R at position E251 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position E251 of SEQ ID NO: 1.

### V252

In some embodiments, a KOD polymerase variant comprises: a C, E, M, T, or W at a position corresponding to V252 of SEQ ID NO: 1. In some embodiments, a KOD polymerase variant comprises: a C, E, M, T, or W at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C, E, M, T, or W at position V252 of SEQ ID NO: 1.

In some embodiments, this disclosure provides a KOD polymerase variant comprising: a C at a position corresponding to V252 of SEQ ID NO: 1. In some embodiments, this disclosure provides a KOD polymerase variant comprising: a C at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position V252 of SEQ ID NO: 1.

In some embodiments, this disclosure provides a KOD polymerase variant comprising: an E at a position corresponding to V252 of SEQ ID NO: 1. In some embodiments, this disclosure provides a KOD polymerase variant comprising: an E at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position V252 of SEQ ID NO: 1.

In some embodiments, this disclosure provides a KOD polymerase variant comprising: a M at a position corresponding to V252 of SEQ ID NO: 1. In some embodiments, this disclosure provides a KOD polymerase variant comprising: a M at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position V252 of SEQ ID NO: 1.

In some embodiments, this disclosure provides a KOD polymerase variant comprising: a T at a position corresponding to V252 of SEQ ID NO: 1. In some embodiments, this disclosure provides a KOD polymerase variant comprising: a T at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position V252 of SEQ ID NO: 1.

In some embodiments, this disclosure provides a KOD polymerase variant comprising: a W at a position corresponding to V252 of SEQ ID NO: 1. In some embodiments, this disclosure provides a KOD polymerase variant comprising: a W at position V252 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position V252 of SEQ ID NO: 1.

### G254

In some embodiments, a KOD polymerase variant comprises: a H, N, or R at a position corresponding to G254 of SEQ ID NO: 1. In some embodiments, a KOD polymerase variant comprises: a H, N, or R at position G254 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H, N, or R at position G254 of SEQ ID NO: 1.

In some embodiments, this disclosure provides a KOD polymerase variant comprising: a H at a position corresponding to G254 of SEQ ID NO: 1. In some embodiments, this disclosure provides a KOD polymerase variant comprising: a H at position G254 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position G254 of SEQ ID NO: 1.

In some embodiments, this disclosure provides a KOD polymerase variant comprising: a N at a position corresponding to G254 of SEQ ID NO: 1. In some embodiments, this disclosure provides a KOD polymerase variant comprising: a N at position G254 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position G254 of SEQ ID NO: 1.

In some embodiments, this disclosure provides a KOD polymerase variant comprising: a R at a position corresponding to G254 of SEQ ID NO: 1. In some embodiments, this disclosure provides a KOD polymerase variant comprising: a R at position G254 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position G254 of SEQ ID NO: 1.

### H257

In some embodiments, a KOD polymerase variant comprises: a F, W, or Y at a position corresponding to H257 of SEQ ID NO: 1. In some embodiments, a KOD polymerase variant comprises: a F, W, or Y at position H257 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F, W, or Y at position H257 of SEQ ID NO: 1.

In some embodiments, this disclosure provides a KOD polymerase variant comprising: a F at a position corresponding to H257 of SEQ ID NO: 1. In some embodiments, this disclosure provides a KOD polymerase variant comprising: a F at position H257 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position H257 of SEQ ID NO: 1.

In some embodiments, this disclosure provides a KOD polymerase variant comprising: a W at a position corresponding to H257 of SEQ ID NO: 1. In some embodiments, this disclosure provides a KOD polymerase variant comprising: a W at position H257 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position H257 of SEQ ID NO: 1.

In some embodiments, this disclosure provides a KOD polymerase variant comprising: a Y at a position corresponding to H257 of SEQ ID NO: 1. In some embodiments, this disclosure provides a KOD polymerase variant comprising: a Y at position H257 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position H257 of SEQ ID NO: 1.

### D259

In some embodiments, a KOD polymerase variant comprises: an A, C, F, G, H, I, K, L, M, N, P, Q, R, S, T, V, W, or Y at a position corresponding to D259 of SEQ ID NO: 1. In some embodiments, a KOD polymerase variant comprises: an A, C, F, G, H, I, K, L, M, N, P, Q, R, S, T, V, W, or Y at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, G, H, I, K, L, M, N, P, Q, R, S, T, V, W, or Y at position D259 of SEQ ID NO: 1.

In some embodiments, this disclosure provides a KOD polymerase variant comprising: an A at a position corresponding to D259 of SEQ ID NO: 1. In some embodiments, this disclosure provides a KOD polymerase variant comprising: an A at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position D259 of SEQ ID NO: 1.

In some embodiments, this disclosure provides a KOD polymerase variant comprising: a C at a position corresponding to D259 of SEQ ID NO: 1. In some embodiments, this disclosure provides a KOD polymerase variant comprising: a C at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position D259 of SEQ ID NO: 1.

In some embodiments, this disclosure provides a KOD polymerase variant comprising: a F at a position corresponding to D259 of SEQ ID NO: 1. In some embodiments, this disclosure provides a KOD polymerase variant comprising: a F at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position D259 of SEQ ID NO: 1.

In some embodiments, this disclosure provides a KOD polymerase variant comprising: a G at a position corresponding to D259 of SEQ ID NO: 1. In some embodiments, this disclosure provides a KOD polymerase variant comprising: a G at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position D259 of SEQ ID NO: 1.

In some embodiments, this disclosure provides a KOD polymerase variant comprising: a H at a position corresponding to D259 of SEQ ID NO: 1. In some embodiments, this disclosure provides a KOD polymerase variant comprising: a H at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position D259 of SEQ ID NO: 1.

In some embodiments, this disclosure provides a KOD polymerase variant comprising: an I at a position corresponding to D259 of SEQ ID NO: 1. In some embodiments, this disclosure provides a KOD polymerase variant comprising: an I at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D259 of SEQ ID NO: 1.

In some embodiments, this disclosure provides a KOD polymerase variant comprising: a K at a position corresponding to D259 of SEQ ID NO: 1. In some embodiments, this disclosure provides a KOD polymerase variant comprising: a K at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D259 of SEQ ID NO: 1.

In some embodiments, this disclosure provides a KOD polymerase variant comprising: a L at a position corresponding to D259 of SEQ ID NO: 1. In some embodiments, this disclosure provides a KOD polymerase variant comprising: a L at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D259 of SEQ ID NO: 1.

In some embodiments, this disclosure provides a KOD polymerase variant comprising: a M at a position corresponding to D259 of SEQ ID NO: 1. In some embodiments, this disclosure provides a KOD polymerase variant comprising: a M at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D259 of SEQ ID NO: 1.

In some embodiments, this disclosure provides a KOD polymerase variant comprising: a N at a position corresponding to D259 of SEQ ID NO: 1. In some embodiments, this disclosure provides a KOD polymerase variant comprising: a N at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position D259 of SEQ ID NO: 1.

In some embodiments, this disclosure provides a KOD polymerase variant comprising: a P at a position corresponding to D259 of SEQ ID NO: 1. In some embodiments, this disclosure provides a KOD polymerase variant comprising: a P at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D259 of SEQ ID NO: 1.

In some embodiments, this disclosure provides a KOD polymerase variant comprising: a Q at a position corresponding to D259 of SEQ ID NO: 1. In some embodiments, this disclosure provides a KOD polymerase variant comprising: a Q at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D259 of SEQ ID NO: 1.

In some embodiments, this disclosure provides a KOD polymerase variant comprising: a R at a position corresponding to D259 of SEQ ID NO: 1. In some embodiments, this disclosure provides a KOD polymerase variant comprising: a R at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position D259 of SEQ ID NO: 1.

In some embodiments, this disclosure provides a KOD polymerase variant comprising: a S at a position corresponding to D259 of SEQ ID NO: 1. In some embodiments, this disclosure provides a KOD polymerase variant comprising: a S at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position D259 of SEQ ID NO: 1.

In some embodiments, this disclosure provides a KOD polymerase variant comprising: a T at a position corresponding to D259 of SEQ ID NO: 1. In some embodiments, this disclosure provides a KOD polymerase variant comprising: a T at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D259 of SEQ ID NO: 1.

In some embodiments, this disclosure provides a KOD polymerase variant comprising: a V at a position corresponding to D259 of SEQ ID NO: 1. In some embodiments, this disclosure provides a KOD polymerase variant comprising: a V at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D259 of SEQ ID NO: 1.

In some embodiments, this disclosure provides a KOD polymerase variant comprising: a W at a position corresponding to D259 of SEQ ID NO: 1. In some embodiments, this disclosure provides a KOD polymerase variant comprising: a W at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position D259 of SEQ ID NO: 1.

In some embodiments, this disclosure provides a KOD polymerase variant comprising: a Y at a position corresponding to D259 of SEQ ID NO: 1. In some embodiments, this disclosure provides a KOD polymerase variant comprising: a Y at position D259 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D259 of SEQ ID NO: 1.

### L260

In some embodiments, this disclosure provides a KOD polymerase variant comprising: a W at a position corresponding to L260 of SEQ ID NO: 1. In some embodiments, this disclosure provides a KOD polymerase variant comprising: a W at position L260 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position L260 of SEQ ID NO: 1.

### Y261

In some embodiments, a KOD polymerase variant comprises: a D, E, or F at a position corresponding to Y261 of SEQ ID NO: 1. In some embodiments, a KOD polymerase variant comprises: a D, E, or F at position Y261 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, or F at position Y261 of SEQ ID NO: 1.

In some embodiments, this disclosure provides a KOD polymerase variant comprising: a D at a position corresponding to Y261 of SEQ ID NO: 1. In some embodiments, this disclosure provides a KOD polymerase variant comprising: a D at position Y261 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y261 of SEQ ID NO: 1.

In some embodiments, this disclosure provides a KOD polymerase variant comprising: an E at a position corresponding to Y261 of SEQ ID NO: 1. In some embodiments, this disclosure provides a KOD polymerase variant comprising: an E at position Y261 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y261 of SEQ ID NO: 1.

In some embodiments, this disclosure provides a KOD polymerase variant comprising: a F at a position corresponding to Y261 of SEQ ID NO: 1. In some embodiments, this disclosure provides a KOD polymerase variant comprising: a F at position Y261 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position Y261 of SEQ ID NO: 1.

### Y273

In some embodiments, a KOD polymerase variant comprises: a D or P at a position corresponding to Y273 of SEQ ID NO: 1. In some embodiments, a KOD polymerase variant comprises: a D or P at position Y273 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or P at position Y273 of SEQ ID NO: 1.

In some embodiments, this disclosure provides a KOD polymerase variant comprising: a D at a position corresponding to Y273 of SEQ ID NO: 1. In some embodiments, this disclosure provides a KOD polymerase variant comprising: a D at position Y273 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y273 of SEQ ID NO: 1.

In some embodiments, this disclosure provides a KOD polymerase variant comprising: a P at a position corresponding to Y273 of SEQ ID NO: 1. In some embodiments, this disclosure provides a KOD polymerase variant comprising: a P at position Y273 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y273 of SEQ ID NO: 1.

### T274

In some embodiments, this disclosure provides a KOD polymerase variant comprising: a D at a position corresponding to T274 of SEQ ID NO: 1. In some embodiments, this disclosure provides a KOD polymerase variant comprising: a D at position T274 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position T274 of SEQ ID NO: 1.

### L275

In some embodiments, a KOD polymerase variant comprises: a H, P, R, or S at a position corresponding to L275 of SEQ ID NO: 1. In some embodiments, a KOD polymerase variant comprises: a H, P, R, or S at position L275 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H, P, R, or S at position L275 of SEQ ID NO: 1.

In some embodiments, this disclosure provides a KOD polymerase variant comprising: a H at a position corresponding to L275 of SEQ ID NO: 1. In some embodiments, this disclosure provides a KOD polymerase variant comprising: a H at position L275 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position L275 of SEQ ID NO: 1.

In some embodiments, this disclosure provides a KOD polymerase variant comprising: a P at a position corresponding to L275 of SEQ ID NO: 1. In some embodiments, this disclosure provides a KOD polymerase variant comprising: a P at position L275 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position L275 of SEQ ID NO: 1.

In some embodiments, this disclosure provides a KOD polymerase variant comprising: a R at a position corresponding to L275 of SEQ ID NO: 1. In some embodiments, this disclosure provides a KOD polymerase variant comprising: a R at position L275 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position L275 of SEQ ID NO: 1.

In some embodiments, this disclosure provides a KOD polymerase variant comprising: a S at a position corresponding to L275 of SEQ ID NO: 1. In some embodiments, this disclosure provides a KOD polymerase variant comprising: a S at position L275 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position L275 of SEQ ID NO: 1.

### A277

In some embodiments, a KOD polymerase variant comprises: an E or P at a position corresponding to A277 of SEQ ID NO: 1. In some embodiments, a KOD polymerase variant comprises: an E or P at position A277 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E or P at position A277 of SEQ ID NO: 1.

In some embodiments, this disclosure provides a KOD polymerase variant comprising: an E at a position corresponding to A277 of SEQ ID NO: 1. In some embodiments, this disclosure provides a KOD polymerase variant comprising: an E at position A277 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A277 of SEQ ID NO: 1.

In some embodiments, this disclosure provides a KOD polymerase variant comprising: a P at a position corresponding to A277 of SEQ ID NO: 1. In some embodiments, this disclosure provides a KOD polymerase variant comprising: a P at position A277 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position A277 of SEQ ID NO: 1.

### V278

In some embodiments, this disclosure provides a KOD polymerase variant comprising: a F at a position corresponding to V278 of SEQ ID NO: 1. In some embodiments, this disclosure provides a KOD polymerase variant comprising: a F at position V278 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position V278 of SEQ ID NO: 1.

### E280

In some embodiments, this disclosure provides a KOD polymerase variant comprising: a M at a position corresponding to E280 of SEQ ID NO: 1. In some embodiments, this disclosure provides a KOD polymerase variant comprising: a M at position E280 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position E280 of SEQ ID NO: 1.

### A281

In some embodiments, this disclosure provides a KOD polymerase variant comprising: a W at a position corresponding to A281 of SEQ ID NO: 1. In some embodiments, this disclosure provides a KOD polymerase variant comprising: a W at position A281 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position A281 of SEQ ID NO: 1.

### K287

In some embodiments, a KOD polymerase variant comprises: a D, E, F, I, M, P, T, or V at a position corresponding to K287 of SEQ ID NO: 1. In some embodiments, a KOD polymerase variant comprises: a D, E, F, I, M, P, T, or V at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, I, M, P, T, or V at position K287 of SEQ ID NO: 1.

In some embodiments, this disclosure provides a KOD polymerase variant comprising: a D at a position corresponding to K287 of SEQ ID NO: 1. In some embodiments, this disclosure provides a KOD polymerase variant comprising: a D at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position K287 of SEQ ID NO: 1.

In some embodiments, this disclosure provides a KOD polymerase variant comprising: an E at a position corresponding to K287 of SEQ ID NO: 1. In some embodiments, this disclosure provides a KOD polymerase variant comprising: an E at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position K287 of SEQ ID NO: 1.

In some embodiments, this disclosure provides a KOD polymerase variant comprising: a F at a position corresponding to K287 of SEQ ID NO: 1. In some embodiments, this disclosure provides a KOD polymerase variant comprising: a F at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position K287 of SEQ ID NO: 1.

In some embodiments, this disclosure provides a KOD polymerase variant comprising: an I at a position corresponding to K287 of SEQ ID NO: 1. In some embodiments, this disclosure provides a KOD polymerase variant comprising: an I at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position K287 of SEQ ID NO: 1.

In some embodiments, this disclosure provides a KOD polymerase variant comprising: a M at a position corresponding to K287 of SEQ ID NO: 1. In some embodiments, this disclosure provides a KOD polymerase variant comprising: a M at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position K287 of SEQ ID NO: 1.

In some embodiments, this disclosure provides a KOD polymerase variant comprising: a P at a position corresponding to K287 of SEQ ID NO: 1. In some embodiments, this disclosure provides a KOD polymerase variant comprising: a P at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position K287 of SEQ ID NO: 1.

In some embodiments, this disclosure provides a KOD polymerase variant comprising: a T at a position corresponding to K287 of SEQ ID NO: 1. In some embodiments, this disclosure provides a KOD polymerase variant comprising: a T at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position K287 of SEQ ID NO: 1.

In some embodiments, this disclosure provides a KOD polymerase variant comprising: a V at a position corresponding to K287 of SEQ ID NO: 1. In some embodiments, this disclosure provides a KOD polymerase variant comprising: a V at position K287 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position K287 of SEQ ID NO: 1.

### K289

In some embodiments, a KOD polymerase variant comprises: an A, D, E, F, G, H, I, L, M, P, Q, R, S, T, V, W, or Y at a position corresponding to K289 of SEQ ID NO: 1. In some embodiments, a KOD polymerase variant comprises: an A, D, E, F, G, H, I, L, M, P, Q, R, S, T, V, W, or Y at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, F, G, H, I, L, M, P, Q, R, S, T, V, W, or Y at position K289 of SEQ ID NO: 1.

In some embodiments, this disclosure provides a KOD polymerase variant comprising: an A at a position corresponding to K289 of SEQ ID NO: 1. In some embodiments, this disclosure provides a KOD polymerase variant comprising: an A at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position K289 of SEQ ID NO: 1.

In some embodiments, this disclosure provides a KOD polymerase variant comprising: a D at a position corresponding to K289 of SEQ ID NO: 1. In some embodiments, this disclosure provides a KOD polymerase variant comprising: a D at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position K289 of SEQ ID NO: 1.

In some embodiments, this disclosure provides a KOD polymerase variant comprising: an E at a position corresponding to K289 of SEQ ID NO: 1. In some embodiments, this disclosure provides a KOD polymerase variant comprising: an E at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position K289 of SEQ ID NO: 1.

In some embodiments, this disclosure provides a KOD polymerase variant comprising: a F at a position corresponding to K289 of SEQ ID NO: 1. In some embodiments, this disclosure provides a KOD polymerase variant comprising: a F at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position K289 of SEQ ID NO: 1.

In some embodiments, this disclosure provides a KOD polymerase variant comprising: a G at a position corresponding to K289 of SEQ ID NO: 1. In some embodiments, this disclosure provides a KOD polymerase variant comprising: a G at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position K289 of SEQ ID NO: 1.

In some embodiments, this disclosure provides a KOD polymerase variant comprising: a H at a position corresponding to K289 of SEQ ID NO: 1. In some embodiments, this disclosure provides a KOD polymerase variant comprising: a H at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position K289 of SEQ ID NO: 1.

In some embodiments, this disclosure provides a KOD polymerase variant comprising: an I at a position corresponding to K289 of SEQ ID NO: 1. In some embodiments, this disclosure provides a KOD polymerase variant comprising: an I at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position K289 of SEQ ID NO: 1.

In some embodiments, this disclosure provides a KOD polymerase variant comprising: a L at a position corresponding to K289 of SEQ ID NO: 1. In some embodiments, this disclosure provides a KOD polymerase variant comprising: a L at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position K289 of SEQ ID NO: 1.

In some embodiments, this disclosure provides a KOD polymerase variant comprising: a M at a position corresponding to K289 of SEQ ID NO: 1. In some embodiments, this disclosure provides a KOD polymerase variant comprising: a M at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position K289 of SEQ ID NO: 1.

In some embodiments, this disclosure provides a KOD polymerase variant comprising: a P at a position corresponding to K289 of SEQ ID NO: 1. In some embodiments, this disclosure provides a KOD polymerase variant comprising: a P at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position K289 of SEQ ID NO: 1.

In some embodiments, this disclosure provides a KOD polymerase variant comprising: a Q at a position corresponding to K289 of SEQ ID NO: 1. In some embodiments, this disclosure provides a KOD polymerase variant comprising: a Q at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position K289 of SEQ ID NO: 1.

In some embodiments, this disclosure provides a KOD polymerase variant comprising: a R at a position corresponding to K289 of SEQ ID NO: 1. In some embodiments, this disclosure provides a KOD polymerase variant comprising: a R at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position K289 of SEQ ID NO: 1.

In some embodiments, this disclosure provides a KOD polymerase variant comprising: a S at a position corresponding to K289 of SEQ ID NO: 1. In some embodiments, this disclosure provides a KOD polymerase variant comprising: a S at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position K289 of SEQ ID NO: 1.

In some embodiments, this disclosure provides a KOD polymerase variant comprising: a T at a position corresponding to K289 of SEQ ID NO: 1. In some embodiments, this disclosure provides a KOD polymerase variant comprising: a T at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position K289 of SEQ ID NO: 1.

In some embodiments, this disclosure provides a KOD polymerase variant comprising: a V at a position corresponding to K289 of SEQ ID NO: 1. In some embodiments, this disclosure provides a KOD polymerase variant comprising: a V at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position K289 of SEQ ID NO: 1.

In some embodiments, this disclosure provides a KOD polymerase variant comprising: a W at a position corresponding to K289 of SEQ ID NO: 1. In some embodiments, this disclosure provides a KOD polymerase variant comprising: a W at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position K289 of SEQ ID NO: 1.

In some embodiments, this disclosure provides a KOD polymerase variant comprising: a Y at a position corresponding to K289 of SEQ ID NO: 1. In some embodiments, this disclosure provides a KOD polymerase variant comprising: a Y at position K289 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position K289 of SEQ ID NO: 1.

### V290

In some embodiments, a KOD polymerase variant comprises: an A, D, E, G, H, K, N, P, Q, R, S, or T at a position corresponding to V290 of SEQ ID NO: 1. In some embodiments, a KOD polymerase variant comprises: an A, D, E, G, H, K, N, P, Q, R, S, or T at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, D, E, G, H, K, N, P, Q, R, S, or T at position V290 of SEQ ID NO: 1.

In some embodiments, this disclosure provides a KOD polymerase variant comprising: an A at a position corresponding to V290 of SEQ ID NO: 1. In some embodiments, this disclosure provides a KOD polymerase variant comprising: an A at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position V290 of SEQ ID NO: 1.

In some embodiments, this disclosure provides a KOD polymerase variant comprising: a D at a position corresponding to V290 of SEQ ID NO: 1. In some embodiments, this disclosure provides a KOD polymerase variant comprising: a D at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position V290 of SEQ ID NO: 1.

In some embodiments, this disclosure provides a KOD polymerase variant comprising: an E at a position corresponding to V290 of SEQ ID NO: 1. In some embodiments, this disclosure provides a KOD polymerase variant comprising: an E at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position V290 of SEQ ID NO: 1.

In some embodiments, this disclosure provides a KOD polymerase variant comprising: a G at a position corresponding to V290 of SEQ ID NO: 1. In some embodiments, this disclosure provides a KOD polymerase variant comprising: a G at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position V290 of SEQ ID NO: 1.

In some embodiments, this disclosure provides a KOD polymerase variant comprising: a H at a position corresponding to V290 of SEQ ID NO: 1. In some embodiments, this disclosure provides a KOD polymerase variant comprising: a H at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position V290 of SEQ ID NO: 1.

In some embodiments, this disclosure provides a KOD polymerase variant comprising: a K at a position corresponding to V290 of SEQ ID NO: 1. In some embodiments, this disclosure provides a KOD polymerase variant comprising: a K at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position V290 of SEQ ID NO: 1.

In some embodiments, this disclosure provides a KOD polymerase variant comprising: a N at a position corresponding to V290 of SEQ ID NO: 1. In some embodiments, this disclosure provides a KOD polymerase variant comprising: a N at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position V290 of SEQ ID NO: 1.

In some embodiments, this disclosure provides a KOD polymerase variant comprising: a P at a position corresponding to V290 of SEQ ID NO: 1. In some embodiments, this disclosure provides a KOD polymerase variant comprising: a P at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position V290 of SEQ ID NO: 1.

In some embodiments, this disclosure provides a KOD polymerase variant comprising: a Q at a position corresponding to V290 of SEQ ID NO: 1. In some embodiments, this disclosure provides a KOD polymerase variant comprising: a Q at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position V290 of SEQ ID NO: 1.

In some embodiments, this disclosure provides a KOD polymerase variant comprising: a R at a position corresponding to V290 of SEQ ID NO: 1. In some embodiments, this disclosure provides a KOD polymerase variant comprising: a R at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position V290 of SEQ ID NO: 1.

In some embodiments, this disclosure provides a KOD polymerase variant comprising: a S at a position corresponding to V290 of SEQ ID NO: 1. In some embodiments, this disclosure provides a KOD polymerase variant comprising: a S at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position V290 of SEQ ID NO: 1.

In some embodiments, this disclosure provides a KOD polymerase variant comprising: a T at a position corresponding to V290 of SEQ ID NO: 1. In some embodiments, this disclosure provides a KOD polymerase variant comprising: a T at position V290 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position V290 of SEQ ID NO: 1.

### A292

In some embodiments, a KOD polymerase variant comprises: a D, E, N, Q, or T at a position corresponding to A292 of SEQ ID NO: 1. In some embodiments, a KOD polymerase variant comprises: a D, E, N, Q, or T at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, N, Q, or T at position A292 of SEQ ID NO: 1.

In some embodiments, this disclosure provides a KOD polymerase variant comprising: a D at a position corresponding to A292 of SEQ ID NO: 1. In some embodiments, this disclosure provides a KOD polymerase variant comprising: a D at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A292 of SEQ ID NO: 1.

In some embodiments, this disclosure provides a KOD polymerase variant comprising: an E at a position corresponding to A292 of SEQ ID NO: 1. In some embodiments, this disclosure provides a KOD polymerase variant comprising: an E at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A292 of SEQ ID NO: 1.

In some embodiments, this disclosure provides a KOD polymerase variant comprising: a N at a position corresponding to A292 of SEQ ID NO: 1. In some embodiments, this disclosure provides a KOD polymerase variant comprising: a N at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position A292 of SEQ ID NO: 1.

In some embodiments, this disclosure provides a KOD polymerase variant comprising: a Q at a position corresponding to A292 of SEQ ID NO: 1. In some embodiments, this disclosure provides a KOD polymerase variant comprising: a Q at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position A292 of SEQ ID NO: 1.

In some embodiments, this disclosure provides a KOD polymerase variant comprising: a T at a position corresponding to A292 of SEQ ID NO: 1. In some embodiments, this disclosure provides a KOD polymerase variant comprising: a T at position A292 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position A292 of SEQ ID NO: 1.

### E294

In some embodiments, a KOD polymerase variant comprises: a K, P, or R at a position corresponding to E294 of SEQ ID NO: 1. In some embodiments, a KOD polymerase variant comprises: a K, P, or R at position E294 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K, P, or R at position E294 of SEQ ID NO: 1.

In some embodiments, this disclosure provides a KOD polymerase variant comprising: a K at a position corresponding to E294 of SEQ ID NO: 1. In some embodiments, this disclosure provides a KOD polymerase variant comprising: a K at position E294 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position E294 of SEQ ID NO: 1.

In some embodiments, this disclosure provides a KOD polymerase variant comprising: a P at a position corresponding to E294 of SEQ ID NO: 1. In some embodiments, this disclosure provides a KOD polymerase variant comprising: a P at position E294 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E294 of SEQ ID NO: 1.

In some embodiments, this disclosure provides a KOD polymerase variant comprising: a R at a position corresponding to E294 of SEQ ID NO: 1. In some embodiments, this disclosure provides a KOD polymerase variant comprising: a R at position E294 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position E294 of SEQ ID NO: 1.

### I295

In some embodiments, a KOD polymerase variant comprises: an A, C, D, E, G, H, K, N, P, Q, R, S, T, W, or Y at a position corresponding to I295 of SEQ ID NO: 1. In some embodiments, a KOD polymerase variant comprises: an A, C, D, E, G, H, K, N, P, Q, R, S, T, W, or Y at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, G, H, K, N, P, Q, R, S, T, W, or Y at position I295 of SEQ ID NO: 1.

In some embodiments, this disclosure provides a KOD polymerase variant comprising: an A at a position corresponding to I295 of SEQ ID NO: 1. In some embodiments, this disclosure provides a KOD polymerase variant comprising: an A at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position I295 of SEQ ID NO: 1.

In some embodiments, this disclosure provides a KOD polymerase variant comprising: a C at a position corresponding to I295 of SEQ ID NO: 1. In some embodiments, this disclosure provides a KOD polymerase variant comprising: a C at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position I295 of SEQ ID NO: 1.

In some embodiments, this disclosure provides a KOD polymerase variant comprising: a D at a position corresponding to I295 of SEQ ID NO: 1. In some embodiments, this disclosure provides a KOD polymerase variant comprising: a D at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position I295 of SEQ ID NO: 1.

In some embodiments, this disclosure provides a KOD polymerase variant comprising: an E at a position corresponding to I295 of SEQ ID NO: 1. In some embodiments, this disclosure provides a KOD polymerase variant comprising: an E at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position I295 of SEQ ID NO: 1.

In some embodiments, this disclosure provides a KOD polymerase variant comprising: a G at a position corresponding to I295 of SEQ ID NO: 1. In some embodiments, this disclosure provides a KOD polymerase variant comprising: a G at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position I295 of SEQ ID NO: 1.

In some embodiments, this disclosure provides a KOD polymerase variant comprising: a H at a position corresponding to I295 of SEQ ID NO: 1. In some embodiments, this disclosure provides a KOD polymerase variant comprising: a H at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position I295 of SEQ ID NO: 1.

In some embodiments, this disclosure provides a KOD polymerase variant comprising: a K at a position corresponding to I295 of SEQ ID NO: 1. In some embodiments, this disclosure provides a KOD polymerase variant comprising: a K at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position I295 of SEQ ID NO: 1.

In some embodiments, this disclosure provides a KOD polymerase variant comprising: a N at a position corresponding to I295 of SEQ ID NO: 1. In some embodiments, this disclosure provides a KOD polymerase variant comprising: a N at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position I295 of SEQ ID NO: 1.

In some embodiments, this disclosure provides a KOD polymerase variant comprising: a P at a position corresponding to I295 of SEQ ID NO: 1. In some embodiments, this disclosure provides a KOD polymerase variant comprising: a P at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position I295 of SEQ ID NO: 1.

In some embodiments, this disclosure provides a KOD polymerase variant comprising: a Q at a position corresponding to I295 of SEQ ID NO: 1. In some embodiments, this disclosure provides a KOD polymerase variant comprising: a Q at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position I295 of SEQ ID NO: 1.

In some embodiments, this disclosure provides a KOD polymerase variant comprising: a R at a position corresponding to I295 of SEQ ID NO: 1. In some embodiments, this disclosure provides a KOD polymerase variant comprising: a R at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position I295 of SEQ ID NO: 1.

In some embodiments, this disclosure provides a KOD polymerase variant comprising: a S at a position corresponding to I295 of SEQ ID NO: 1. In some embodiments, this disclosure provides a KOD polymerase variant comprising: a S at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position I295 of SEQ ID NO: 1.

In some embodiments, this disclosure provides a KOD polymerase variant comprising: a T at a position corresponding to I295 of SEQ ID NO: 1. In some embodiments, this disclosure provides a KOD polymerase variant comprising: a T at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position I295 of SEQ ID NO: 1.

In some embodiments, this disclosure provides a KOD polymerase variant comprising: a W at a position corresponding to I295 of SEQ ID NO: 1. In some embodiments, this disclosure provides a KOD polymerase variant comprising: a W at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position I295 of SEQ ID NO: 1.

In some embodiments, this disclosure provides a KOD polymerase variant comprising: a Y at a position corresponding to I295 of SEQ ID NO: 1. In some embodiments, this disclosure provides a KOD polymerase variant comprising: a Y at position I295 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position I295 of SEQ ID NO: 1.

### A298

In some embodiments, a KOD polymerase variant comprises: a D or P at a position corresponding to A298 of SEQ ID NO: 1. In some embodiments, a KOD polymerase variant comprises: a D or P at position A298 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D or P at position A298 of SEQ ID NO: 1.

In some embodiments, this disclosure provides a KOD polymerase variant comprising: a D at a position corresponding to A298 of SEQ ID NO: 1. In some embodiments, this disclosure provides a KOD polymerase variant comprising: a D at position A298 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A298 of SEQ ID NO: 1.

In some embodiments, this disclosure provides a KOD polymerase variant comprising: a P at a position corresponding to A298 of SEQ ID NO: 1. In some embodiments, this disclosure provides a KOD polymerase variant comprising: a P at position A298 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position A298 of SEQ ID NO: 1.

### W299

In some embodiments, a KOD polymerase variant comprises: a D, E, G, H, I, K, L, M, N, P, Q, R, T, or V at a position corresponding to W299 of SEQ ID NO: 1. In some embodiments, a KOD polymerase variant comprises: a D, E, G, H, I, K, L, M, N, P, Q, R, T, or V at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, G, H, I, K, L, M, N, P, Q, R, T, or V at position W299 of SEQ ID NO: 1.

In some embodiments, this disclosure provides a KOD polymerase variant comprising: a D at a position corresponding to W299 of SEQ ID NO: 1. In some embodiments, this disclosure provides a KOD polymerase variant comprising: a D at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position W299 of SEQ ID NO: 1.

In some embodiments, this disclosure provides a KOD polymerase variant comprising: an E at a position corresponding to W299 of SEQ ID NO: 1. In some embodiments, this disclosure provides a KOD polymerase variant comprising: an E at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position W299 of SEQ ID NO: 1.

In some embodiments, this disclosure provides a KOD polymerase variant comprising: a G at a position corresponding to W299 of SEQ ID NO: 1. In some embodiments, this disclosure provides a KOD polymerase variant comprising: a G at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position W299 of SEQ ID NO: 1.

In some embodiments, this disclosure provides a KOD polymerase variant comprising: a H at a position corresponding to W299 of SEQ ID NO: 1. In some embodiments, this disclosure provides a KOD polymerase variant comprising: a H at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position W299 of SEQ ID NO: 1.

In some embodiments, this disclosure provides a KOD polymerase variant comprising: an I at a position corresponding to W299 of SEQ ID NO: 1. In some embodiments, this disclosure provides a KOD polymerase variant comprising: an I at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position W299 of SEQ ID NO: 1.

In some embodiments, this disclosure provides a KOD polymerase variant comprising: a K at a position corresponding to W299 of SEQ ID NO: 1. In some embodiments, this disclosure provides a KOD polymerase variant comprising: a K at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position W299 of SEQ ID NO: 1.

In some embodiments, this disclosure provides a KOD polymerase variant comprising: a L at a position corresponding to W299 of SEQ ID NO: 1. In some embodiments, this disclosure provides a KOD polymerase variant comprising: a L at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position W299 of SEQ ID NO: 1.

In some embodiments, this disclosure provides a KOD polymerase variant comprising: a M at a position corresponding to W299 of SEQ ID NO: 1. In some embodiments, this disclosure provides a KOD polymerase variant comprising: a M at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position W299 of SEQ ID NO: 1.

In some embodiments, this disclosure provides a KOD polymerase variant comprising: a N at a position corresponding to W299 of SEQ ID NO: 1. In some embodiments, this disclosure provides a KOD polymerase variant comprising: a N at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position W299 of SEQ ID NO: 1.

In some embodiments, this disclosure provides a KOD polymerase variant comprising: a P at a position corresponding to W299 of SEQ ID NO: 1. In some embodiments, this disclosure provides a KOD polymerase variant comprising: a P at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position W299 of SEQ ID NO: 1.

In some embodiments, this disclosure provides a KOD polymerase variant comprising: a Q at a position corresponding to W299 of SEQ ID NO: 1. In some embodiments, this disclosure provides a KOD polymerase variant comprising: a Q at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position W299 of SEQ ID NO: 1.

In some embodiments, this disclosure provides a KOD polymerase variant comprising: a R at a position corresponding to W299 of SEQ ID NO: 1. In some embodiments, this disclosure provides a KOD polymerase variant comprising: a R at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position W299 of SEQ ID NO: 1.

In some embodiments, this disclosure provides a KOD polymerase variant comprising: a T at a position corresponding to W299 of SEQ ID NO: 1. In some embodiments, this disclosure provides a KOD polymerase variant comprising: a T at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position W299 of SEQ ID NO: 1.

In some embodiments, this disclosure provides a KOD polymerase variant comprising: a V at a position corresponding to W299 of SEQ ID NO: 1. In some embodiments, this disclosure provides a KOD polymerase variant comprising: a V at position W299 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position W299 of SEQ ID NO: 1.

### E300

In some embodiments, this disclosure provides a KOD polymerase variant comprising: a P at a position corresponding to E300 of SEQ ID NO: 1. In some embodiments, this disclosure provides a KOD polymerase variant comprising: a P at position E300 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E300 of SEQ ID NO: 1.

### T301

In some embodiments, this disclosure provides a KOD polymerase variant comprising: a P at a position corresponding to T301 of SEQ ID NO: 1. In some embodiments, this disclosure provides a KOD polymerase variant comprising: a P at position T301 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position T301 of SEQ ID NO: 1.

### G302

In some embodiments, this disclosure provides a KOD polymerase variant comprising: a P at a position corresponding to G302 of SEQ ID NO: 1. In some embodiments, this disclosure provides a KOD polymerase variant comprising: a P at position G302 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position G302 of SEQ ID NO: 1.

### L305

In some embodiments, this disclosure provides a KOD polymerase variant comprising: a H at a position corresponding to L305 of SEQ ID NO: 1. In some embodiments, this disclosure provides a KOD polymerase variant comprising: a H at position L305 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position L305 of SEQ ID NO: 1.

### V308

In some embodiments, a KOD polymerase variant comprises: a D, F, H, K, L, N, Q, R, W, or Y at a position corresponding to V308 of SEQ ID NO: 1. In some embodiments, a KOD polymerase variant comprises: a D, F, H, K, L, N, Q, R, W, or Y at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, F, H, K, L, N, Q, R, W, or Y at position V308 of SEQ ID NO: 1.

In some embodiments, this disclosure provides a KOD polymerase variant comprising: a D at a position corresponding to V308 of SEQ ID NO: 1. In some embodiments, this disclosure provides a KOD polymerase variant comprising: a D at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position V308 of SEQ ID NO: 1.

In some embodiments, this disclosure provides a KOD polymerase variant comprising: a F at a position corresponding to V308 of SEQ ID NO: 1. In some embodiments, this disclosure provides a KOD polymerase variant comprising: a F at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position V308 of SEQ ID NO: 1.

In some embodiments, this disclosure provides a KOD polymerase variant comprising: a H at a position corresponding to V308 of SEQ ID NO: 1. In some embodiments, this disclosure provides a KOD polymerase variant comprising: a H at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position V308 of SEQ ID NO: 1.

In some embodiments, this disclosure provides a KOD polymerase variant comprising: a K at a position corresponding to V308 of SEQ ID NO: 1. In some embodiments, this disclosure provides a KOD polymerase variant comprising: a K at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position V308 of SEQ ID NO: 1.

In some embodiments, this disclosure provides a KOD polymerase variant comprising: a L at a position corresponding to V308 of SEQ ID NO: 1. In some embodiments, this disclosure provides a KOD polymerase variant comprising: a L at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position V308 of SEQ ID NO: 1.

In some embodiments, this disclosure provides a KOD polymerase variant comprising: a N at a position corresponding to V308 of SEQ ID NO: 1. In some embodiments, this disclosure provides a KOD polymerase variant comprising: a N at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position V308 of SEQ ID NO: 1.

In some embodiments, this disclosure provides a KOD polymerase variant comprising: a Q at a position corresponding to V308 of SEQ ID NO: 1. In some embodiments, this disclosure provides a KOD polymerase variant comprising: a Q at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position V308 of SEQ ID NO: 1.

In some embodiments, this disclosure provides a KOD polymerase variant comprising: a R at a position corresponding to V308 of SEQ ID NO: 1. In some embodiments, this disclosure provides a KOD polymerase variant comprising: a R at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position V308 of SEQ ID NO: 1.

In some embodiments, this disclosure provides a KOD polymerase variant comprising: a W at a position corresponding to V308 of SEQ ID NO: 1. In some embodiments, this disclosure provides a KOD polymerase variant comprising: a W at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position V308 of SEQ ID NO: 1.

In some embodiments, this disclosure provides a KOD polymerase variant comprising: a Y at a position corresponding to V308 of SEQ ID NO: 1. In some embodiments, this disclosure provides a KOD polymerase variant comprising: a Y at position V308 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position V308 of SEQ ID NO: 1.

### A309

In some embodiments, a KOD polymerase variant comprises: a D, P, or R at a position corresponding to A309 of SEQ ID NO: 1. In some embodiments, a KOD polymerase variant comprises: a D, P, or R at position A309 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, P, or R at position A309 of SEQ ID NO: 1.

In some embodiments, this disclosure provides a KOD polymerase variant comprising: a D at a position corresponding to A309 of SEQ ID NO: 1. In some embodiments, this disclosure provides a KOD polymerase variant comprising: a D at position A309 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A309 of SEQ ID NO: 1.

In some embodiments, this disclosure provides a KOD polymerase variant comprising: a P at a position corresponding to A309 of SEQ ID NO: 1. In some embodiments, this disclosure provides a KOD polymerase variant comprising: a P at position A309 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position A309 of SEQ ID NO: 1.

In some embodiments, this disclosure provides a KOD polymerase variant comprising: a R at a position corresponding to A309 of SEQ ID NO: 1. In some embodiments, this disclosure provides a KOD polymerase variant comprising: a R at position A309 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position A309 of SEQ ID NO: 1.

### Y311

In some embodiments, a KOD polymerase variant comprises: an A, C, D, E, F, G, H, I, K, L, M, N, P, Q, R, S, T, V, or W at a position corresponding to Y311 of SEQ ID NO: 1. In some embodiments, a KOD polymerase variant comprises: an A, C, D, E, F, G, H, I, K, L, M, N, P, Q, R, S, T, V, or W at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, D, E, F, G, H, I, K, L, M, N, P, Q, R, S, T, V, or W at position Y311 of SEQ ID NO: 1.

In some embodiments, this disclosure provides a KOD polymerase variant comprising: an A at a position corresponding to Y311 of SEQ ID NO: 1. In some embodiments, this disclosure provides a KOD polymerase variant comprising: an A at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position Y311 of SEQ ID NO: 1.

In some embodiments, this disclosure provides a KOD polymerase variant comprising: a C at a position corresponding to Y311 of SEQ ID NO: 1. In some embodiments, this disclosure provides a KOD polymerase variant comprising: a C at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position Y311 of SEQ ID NO: 1.

In some embodiments, this disclosure provides a KOD polymerase variant comprising: a D at a position corresponding to Y311 of SEQ ID NO: 1. In some embodiments, this disclosure provides a KOD polymerase variant comprising: a D at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position Y311 of SEQ ID NO: 1.

In some embodiments, this disclosure provides a KOD polymerase variant comprising: an E at a position corresponding to Y311 of SEQ ID NO: 1. In some embodiments, this disclosure provides a KOD polymerase variant comprising: an E at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position Y311 of SEQ ID NO: 1.

In some embodiments, this disclosure provides a KOD polymerase variant comprising: a F at a position corresponding to Y311 of SEQ ID NO: 1. In some embodiments, this disclosure provides a KOD polymerase variant comprising: a F at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position Y311 of SEQ ID NO: 1.

In some embodiments, this disclosure provides a KOD polymerase variant comprising: a G at a position corresponding to Y311 of SEQ ID NO: 1. In some embodiments, this disclosure provides a KOD polymerase variant comprising: a G at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position Y311 of SEQ ID NO: 1.

In some embodiments, this disclosure provides a KOD polymerase variant comprising: a H at a position corresponding to Y311 of SEQ ID NO: 1. In some embodiments, this disclosure provides a KOD polymerase variant comprising: a H at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position Y311 of SEQ ID NO: 1.

In some embodiments, this disclosure provides a KOD polymerase variant comprising: an I at a position corresponding to Y311 of SEQ ID NO: 1. In some embodiments, this disclosure provides a KOD polymerase variant comprising: an I at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position Y311 of SEQ ID NO: 1.

In some embodiments, this disclosure provides a KOD polymerase variant comprising: a K at a position corresponding to Y311 of SEQ ID NO: 1. In some embodiments, this disclosure provides a KOD polymerase variant comprising: a K at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position Y311 of SEQ ID NO: 1.

In some embodiments, this disclosure provides a KOD polymerase variant comprising: a L at a position corresponding to Y311 of SEQ ID NO: 1. In some embodiments, this disclosure provides a KOD polymerase variant comprising: a L at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position Y311 of SEQ ID NO: 1.

In some embodiments, this disclosure provides a KOD polymerase variant comprising: a M at a position corresponding to Y311 of SEQ ID NO: 1. In some embodiments, this disclosure provides a KOD polymerase variant comprising: a M at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position Y311 of SEQ ID NO: 1.

In some embodiments, this disclosure provides a KOD polymerase variant comprising: a N at a position corresponding to Y311 of SEQ ID NO: 1. In some embodiments, this disclosure provides a KOD polymerase variant comprising: a N at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position Y311 of SEQ ID NO: 1.

In some embodiments, this disclosure provides a KOD polymerase variant comprising: a P at a position corresponding to Y311 of SEQ ID NO: 1. In some embodiments, this disclosure provides a KOD polymerase variant comprising: a P at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position Y311 of SEQ ID NO: 1.

In some embodiments, this disclosure provides a KOD polymerase variant comprising: a Q at a position corresponding to Y311 of SEQ ID NO: 1. In some embodiments, this disclosure provides a KOD polymerase variant comprising: a Q at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position Y311 of SEQ ID NO: 1.

In some embodiments, this disclosure provides a KOD polymerase variant comprising: a R at a position corresponding to Y311 of SEQ ID NO: 1. In some embodiments, this disclosure provides a KOD polymerase variant comprising: a R at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position Y311 of SEQ ID NO: 1.

In some embodiments, this disclosure provides a KOD polymerase variant comprising: a S at a position corresponding to Y311 of SEQ ID NO: 1. In some embodiments, this disclosure provides a KOD polymerase variant comprising: a S at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position Y311 of SEQ ID NO: 1.

In some embodiments, this disclosure provides a KOD polymerase variant comprising: a T at a position corresponding to Y311 of SEQ ID NO: 1. In some embodiments, this disclosure provides a KOD polymerase variant comprising: a T at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position Y311 of SEQ ID NO: 1.

In some embodiments, this disclosure provides a KOD polymerase variant comprising: a V at a position corresponding to Y311 of SEQ ID NO: 1. In some embodiments, this disclosure provides a KOD polymerase variant comprising: a V at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position Y311 of SEQ ID NO: 1.

In some embodiments, this disclosure provides a KOD polymerase variant comprising: a W at a position corresponding to Y311 of SEQ ID NO: 1. In some embodiments, this disclosure provides a KOD polymerase variant comprising: a W at position Y311 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position Y311 of SEQ ID NO: 1.

### S312

In some embodiments, a KOD polymerase variant comprises: a D, E, F, H, I, K, L, M, N, P, Q, R, V, W, or Y at a position corresponding to S312 of SEQ ID NO: 1. In some embodiments, a KOD polymerase variant comprises: a D, E, F, H, I, K, L, M, N, P, Q, R, V, W, or Y at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, H, I, K, L, M, N, P, Q, R, V, W, or Y at position S312 of SEQ ID NO: 1.

In some embodiments, this disclosure provides a KOD polymerase variant comprising: a D at a position corresponding to S312 of SEQ ID NO: 1. In some embodiments, this disclosure provides a KOD polymerase variant comprising: a D at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position S312 of SEQ ID NO: 1.

In some embodiments, this disclosure provides a KOD polymerase variant comprising: an E at a position corresponding to S312 of SEQ ID NO: 1. In some embodiments, this disclosure provides a KOD polymerase variant comprising: an E at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position S312 of SEQ ID NO: 1.

In some embodiments, this disclosure provides a KOD polymerase variant comprising: a F at a position corresponding to S312 of SEQ ID NO: 1. In some embodiments, this disclosure provides a KOD polymerase variant comprising: a F at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position S312 of SEQ ID NO: 1.

In some embodiments, this disclosure provides a KOD polymerase variant comprising: a H at a position corresponding to S312 of SEQ ID NO: 1. In some embodiments, this disclosure provides a KOD polymerase variant comprising: a H at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position S312 of SEQ ID NO: 1.

In some embodiments, this disclosure provides a KOD polymerase variant comprising: an I at a position corresponding to S312 of SEQ ID NO: 1. In some embodiments, this disclosure provides a KOD polymerase variant comprising: an I at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position S312 of SEQ ID NO: 1.

In some embodiments, this disclosure provides a KOD polymerase variant comprising: a K at a position corresponding to S312 of SEQ ID NO: 1. In some embodiments, this disclosure provides a KOD polymerase variant comprising: a K at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position S312 of SEQ ID NO: 1.

In some embodiments, this disclosure provides a KOD polymerase variant comprising: a L at a position corresponding to S312 of SEQ ID NO: 1. In some embodiments, this disclosure provides a KOD polymerase variant comprising: a L at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position S312 of SEQ ID NO: 1.

In some embodiments, this disclosure provides a KOD polymerase variant comprising: a M at a position corresponding to S312 of SEQ ID NO: 1. In some embodiments, this disclosure provides a KOD polymerase variant comprising: a M at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position S312 of SEQ ID NO: 1.

In some embodiments, this disclosure provides a KOD polymerase variant comprising: a N at a position corresponding to S312 of SEQ ID NO: 1. In some embodiments, this disclosure provides a KOD polymerase variant comprising: a N at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position S312 of SEQ ID NO: 1.

In some embodiments, this disclosure provides a KOD polymerase variant comprising: a P at a position corresponding to S312 of SEQ ID NO: 1. In some embodiments, this disclosure provides a KOD polymerase variant comprising: a P at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position S312 of SEQ ID NO: 1.

In some embodiments, this disclosure provides a KOD polymerase variant comprising: a Q at a position corresponding to S312 of SEQ ID NO: 1. In some embodiments, this disclosure provides a KOD polymerase variant comprising: a Q at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position S312 of SEQ ID NO: 1.

In some embodiments, this disclosure provides a KOD polymerase variant comprising: a R at a position corresponding to S312 of SEQ ID NO: 1. In some embodiments, this disclosure provides a KOD polymerase variant comprising: a R at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position S312 of SEQ ID NO: 1.

In some embodiments, this disclosure provides a KOD polymerase variant comprising: a V at a position corresponding to S312 of SEQ ID NO: 1. In some embodiments, this disclosure provides a KOD polymerase variant comprising: a V at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position S312 of SEQ ID NO: 1.

In some embodiments, this disclosure provides a KOD polymerase variant comprising: a W at a position corresponding to S312 of SEQ ID NO: 1. In some embodiments, this disclosure provides a KOD polymerase variant comprising: a W at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position S312 of SEQ ID NO: 1.

In some embodiments, this disclosure provides a KOD polymerase variant comprising: a Y at a position corresponding to S312 of SEQ ID NO: 1. In some embodiments, this disclosure provides a KOD polymerase variant comprising: a Y at position S312 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position S312 of SEQ ID NO: 1.

### M313

In some embodiments, a KOD polymerase variant comprises: a P or Q at a position corresponding to M313 of SEQ ID NO: 1. In some embodiments, a KOD polymerase variant comprises: a P or Q at position M313 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P or Q at position M313 of SEQ ID NO: 1.

In some embodiments, this disclosure provides a KOD polymerase variant comprising: a P at a position corresponding to M313 of SEQ ID NO: 1. In some embodiments, this disclosure provides a KOD polymerase variant comprising: a P at position M313 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position M313 of SEQ ID NO: 1.

In some embodiments, this disclosure provides a KOD polymerase variant comprising: a Q at a position corresponding to M313 of SEQ ID NO: 1. In some embodiments, this disclosure provides a KOD polymerase variant comprising: a Q at position M313 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position M313 of SEQ ID NO: 1.

### E314

In some embodiments, this disclosure provides a KOD polymerase variant comprising: a P at a position corresponding to E314 of SEQ ID NO: 1. In some embodiments, this disclosure provides a KOD polymerase variant comprising: a P at position E314 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position E314 of SEQ ID NO: 1.

### D315

In some embodiments, a KOD polymerase variant comprises: an A, C, F, G, I, K, L, M, N, P, Q, R, S, T, V, W, or Y at a position corresponding to D315 of SEQ ID NO: 1. In some embodiments, a KOD polymerase variant comprises: an A, C, F, G, I, K, L, M, N, P, Q, R, S, T, V, W, or Y at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A, C, F, G, I, K, L, M, N, P, Q, R, S, T, V, W, or Y at position D315 of SEQ ID NO: 1.

In some embodiments, this disclosure provides a KOD polymerase variant comprising: an A at a position corresponding to D315 of SEQ ID NO: 1. In some embodiments, this disclosure provides a KOD polymerase variant comprising: an A at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an A at position D315 of SEQ ID NO: 1.

In some embodiments, this disclosure provides a KOD polymerase variant comprising: a C at a position corresponding to D315 of SEQ ID NO: 1. In some embodiments, this disclosure provides a KOD polymerase variant comprising: a C at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a C at position D315 of SEQ ID NO: 1.

In some embodiments, this disclosure provides a KOD polymerase variant comprising: a F at a position corresponding to D315 of SEQ ID NO: 1. In some embodiments, this disclosure provides a KOD polymerase variant comprising: a F at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position D315 of SEQ ID NO: 1.

In some embodiments, this disclosure provides a KOD polymerase variant comprising: a G at a position corresponding to D315 of SEQ ID NO: 1. In some embodiments, this disclosure provides a KOD polymerase variant comprising: a G at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a G at position D315 of SEQ ID NO: 1.

In some embodiments, this disclosure provides a KOD polymerase variant comprising: an I at a position corresponding to D315 of SEQ ID NO: 1. In some embodiments, this disclosure provides a KOD polymerase variant comprising: an I at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position D315 of SEQ ID NO: 1.

In some embodiments, this disclosure provides a KOD polymerase variant comprising: a K at a position corresponding to D315 of SEQ ID NO: 1. In some embodiments, this disclosure provides a KOD polymerase variant comprising: a K at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position D315 of SEQ ID NO: 1.

In some embodiments, this disclosure provides a KOD polymerase variant comprising: a L at a position corresponding to D315 of SEQ ID NO: 1. In some embodiments, this disclosure provides a KOD polymerase variant comprising: a L at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position D315 of SEQ ID NO: 1.

In some embodiments, this disclosure provides a KOD polymerase variant comprising: a M at a position corresponding to D315 of SEQ ID NO: 1. In some embodiments, this disclosure provides a KOD polymerase variant comprising: a M at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position D315 of SEQ ID NO: 1.

In some embodiments, this disclosure provides a KOD polymerase variant comprising: a N at a position corresponding to D315 of SEQ ID NO: 1. In some embodiments, this disclosure provides a KOD polymerase variant comprising: a N at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position D315 of SEQ ID NO: 1.

In some embodiments, this disclosure provides a KOD polymerase variant comprising: a P at a position corresponding to D315 of SEQ ID NO: 1. In some embodiments, this disclosure provides a KOD polymerase variant comprising: a P at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position D315 of SEQ ID NO: 1.

In some embodiments, this disclosure provides a KOD polymerase variant comprising: a Q at a position corresponding to D315 of SEQ ID NO: 1. In some embodiments, this disclosure provides a KOD polymerase variant comprising: a Q at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position D315 of SEQ ID NO: 1.

In some embodiments, this disclosure provides a KOD polymerase variant comprising: a R at a position corresponding to D315 of SEQ ID NO: 1. In some embodiments, this disclosure provides a KOD polymerase variant comprising: a R at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position D315 of SEQ ID NO: 1.

In some embodiments, this disclosure provides a KOD polymerase variant comprising: a S at a position corresponding to D315 of SEQ ID NO: 1. In some embodiments, this disclosure provides a KOD polymerase variant comprising: a S at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a S at position D315 of SEQ ID NO: 1.

In some embodiments, this disclosure provides a KOD polymerase variant comprising: a T at a position corresponding to D315 of SEQ ID NO: 1. In some embodiments, this disclosure provides a KOD polymerase variant comprising: a T at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a T at position D315 of SEQ ID NO: 1.

In some embodiments, this disclosure provides a KOD polymerase variant comprising: a V at a position corresponding to D315 of SEQ ID NO: 1. In some embodiments, this disclosure provides a KOD polymerase variant comprising: a V at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position D315 of SEQ ID NO: 1.

In some embodiments, this disclosure provides a KOD polymerase variant comprising: a W at a position corresponding to D315 of SEQ ID NO: 1. In some embodiments, this disclosure provides a KOD polymerase variant comprising: a W at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position D315 of SEQ ID NO: 1.

In some embodiments, this disclosure provides a KOD polymerase variant comprising: a Y at a position corresponding to D315 of SEQ ID NO: 1. In some embodiments, this disclosure provides a KOD polymerase variant comprising: a Y at position D315 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position D315 of SEQ ID NO: 1.

### A316

In some embodiments, a KOD polymerase variant comprises: a D, E, F, H, M, N, Q, R, W, or Y at a position corresponding to A316 of SEQ ID NO: 1. In some embodiments, a KOD polymerase variant comprises: a D, E, F, H, M, N, Q, R, W, or Y at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, E, F, H, M, N, Q, R, W, or Y at position A316 of SEQ ID NO: 1.

In some embodiments, this disclosure provides a KOD polymerase variant comprising: a D at a position corresponding to A316 of SEQ ID NO: 1. In some embodiments, this disclosure provides a KOD polymerase variant comprising: a D at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position A316 of SEQ ID NO: 1.

In some embodiments, this disclosure provides a KOD polymerase variant comprising: an E at a position corresponding to A316 of SEQ ID NO: 1. In some embodiments, this disclosure provides a KOD polymerase variant comprising: an E at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an E at position A316 of SEQ ID NO: 1.

In some embodiments, this disclosure provides a KOD polymerase variant comprising: a F at a position corresponding to A316 of SEQ ID NO: 1. In some embodiments, this disclosure provides a KOD polymerase variant comprising: a F at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a F at position A316 of SEQ ID NO: 1.

In some embodiments, this disclosure provides a KOD polymerase variant comprising: a H at a position corresponding to A316 of SEQ ID NO: 1. In some embodiments, this disclosure provides a KOD polymerase variant comprising: a H at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a H at position A316 of SEQ ID NO: 1.

In some embodiments, this disclosure provides a KOD polymerase variant comprising: a M at a position corresponding to A316 of SEQ ID NO: 1. In some embodiments, this disclosure provides a KOD polymerase variant comprising: a M at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position A316 of SEQ ID NO: 1.

In some embodiments, this disclosure provides a KOD polymerase variant comprising: a N at a position corresponding to A316 of SEQ ID NO: 1. In some embodiments, this disclosure provides a KOD polymerase variant comprising: a N at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a N at position A316 of SEQ ID NO: 1.

In some embodiments, this disclosure provides a KOD polymerase variant comprising: a Q at a position corresponding to A316 of SEQ ID NO: 1. In some embodiments, this disclosure provides a KOD polymerase variant comprising: a Q at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position A316 of SEQ ID NO: 1.

In some embodiments, this disclosure provides a KOD polymerase variant comprising: a R at a position corresponding to A316 of SEQ ID NO: 1. In some embodiments, this disclosure provides a KOD polymerase variant comprising: a R at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position A316 of SEQ ID NO: 1.

In some embodiments, this disclosure provides a KOD polymerase variant comprising: a W at a position corresponding to A316 of SEQ ID NO: 1. In some embodiments, this disclosure provides a KOD polymerase variant comprising: a W at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a W at position A316 of SEQ ID NO: 1.

In some embodiments, this disclosure provides a KOD polymerase variant comprising: a Y at a position corresponding to A316 of SEQ ID NO: 1. In some embodiments, this disclosure provides a KOD polymerase variant comprising: a Y at position A316 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Y at position A316 of SEQ ID NO: 1.

### K317

In some embodiments, this disclosure provides a KOD polymerase variant comprising: a P at a position corresponding to K317 of SEQ ID NO: 1. In some embodiments, this disclosure provides a KOD polymerase variant comprising: a P at position K317 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position K317 of SEQ ID NO: 1.

### V318

In some embodiments, a KOD polymerase variant comprises: a P or R at a position corresponding to V318 of SEQ ID NO: 1. In some embodiments, a KOD polymerase variant comprises: a P or R at position V318 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P or R at position V318 of SEQ ID NO: 1.

In some embodiments, this disclosure provides a KOD polymerase variant comprising: a P at a position corresponding to V318 of SEQ ID NO: 1. In some embodiments, this disclosure provides a KOD polymerase variant comprising: a P at position V318 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a P at position V318 of SEQ ID NO: 1.

In some embodiments, this disclosure provides a KOD polymerase variant comprising: a R at a position corresponding to V318 of SEQ ID NO: 1. In some embodiments, this disclosure provides a KOD polymerase variant comprising: a R at position V318 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position V318 of SEQ ID NO: 1.

### T319

In some embodiments, a KOD polymerase variant comprises: a D, I, K, L, M, Q, R, or V at a position corresponding to T319 of SEQ ID NO: 1. In some embodiments, a KOD polymerase variant comprises: a D, I, K, L, M, Q, R, or V at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D, I, K, L, M, Q, R, or V at position T319 of SEQ ID NO: 1.

In some embodiments, this disclosure provides a KOD polymerase variant comprising: a D at a position corresponding to T319 of SEQ ID NO: 1. In some embodiments, this disclosure provides a KOD polymerase variant comprising: a D at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a D at position T319 of SEQ ID NO: 1.

In some embodiments, this disclosure provides a KOD polymerase variant comprising: an I at a position corresponding to T319 of SEQ ID NO: 1. In some embodiments, this disclosure provides a KOD polymerase variant comprising: an I at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and an I at position T319 of SEQ ID NO: 1.

In some embodiments, this disclosure provides a KOD polymerase variant comprising: a K at a position corresponding to T319 of SEQ ID NO: 1. In some embodiments, this disclosure provides a KOD polymerase variant comprising: a K at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a K at position T319 of SEQ ID NO: 1.

In some embodiments, this disclosure provides a KOD polymerase variant comprising: a L at a position corresponding to T319 of SEQ ID NO: 1. In some embodiments, this disclosure provides a KOD polymerase variant comprising: a L at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a L at position T319 of SEQ ID NO: 1.

In some embodiments, this disclosure provides a KOD polymerase variant comprising: a M at a position corresponding to T319 of SEQ ID NO: 1. In some embodiments, this disclosure provides a KOD polymerase variant comprising: a M at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a M at position T319 of SEQ ID NO: 1.

In some embodiments, this disclosure provides a KOD polymerase variant comprising: a Q at a position corresponding to T319 of SEQ ID NO: 1. In some embodiments, this disclosure provides a KOD polymerase variant comprising: a Q at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a Q at position T319 of SEQ ID NO: 1.

In some embodiments, this disclosure provides a KOD polymerase variant comprising: a R at a position corresponding to T319 of SEQ ID NO: 1. In some embodiments, this disclosure provides a KOD polymerase variant comprising: a R at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a R at position T319 of SEQ ID NO: 1.

In some embodiments, this disclosure provides a KOD polymerase variant comprising: a V at a position corresponding to T319 of SEQ ID NO: 1. In some embodiments, this disclosure provides a KOD polymerase variant comprising: a V at position T319 of SEQ ID NO: 1 and one, two, three or four additional mutations (e.g., amino acid substitutions) in SEQ ID NO: 1. In some embodiments, a KOD polymerase variant comprises: an amino acid sequence having at least 90% (e.g., at least 95%, at least 98%, or at least 99%) identity with SEQ ID NO: 1 and a V at position T319 of SEQ ID NO: 1.

### 7p6s Assay Variants

### I16F

In some embodiments, a KOD polymerase variant comprises: a F at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1. In some embodiments, a KOD polymerase variant comprises: a F at position I16 of SEQ ID NO: 1.

### I16F_P36A

In some embodiments, a KOD polymerase variant comprises: a F at a position corresponding to I16 of SEQ ID NO: 1 and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and an A at position P36 of SEQ ID NO: 1. In some embodiments, a KOD polymerase variant comprises: a F at position I16 of SEQ ID NO: 1, and an A at position P36 of SEQ ID NO: 1.

### I16F_P36A_P90F

In some embodiments, a KOD polymerase variant comprises: a F at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a F at position P90 of SEQ ID NO: 1. In some embodiments, a KOD polymerase variant comprises: a F at position I16 of SEQ ID NO: 1, an A at position P36 of SEQ ID NO: 1, and a F at position P90 of SEQ ID NO: 1.

### I16F_P36A_P90F_R97A

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, and an A at position R97 of SEQ ID NO: 1.

### I16F_P36A_P90F_R97F

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, and a F at position R97 of SEQ ID NO: 1.

### I16F_P36A_P90F_V93I

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, and an I at position V93 of SEQ ID NO: 1.

### I16F_P36A_P90F_V93I_R97A

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1.

### I16F_P36A_P90F_V93I_R97F

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1.

### I16F_P36A_P90F_V93I_R97H

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### I16F_P36A_P90F_V93R_R97A

In some embodiments, a KOD polymerase variant comprises: a F at a position corresponding to I16 of SEQ ID NO: 1, an A at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a F at position P90 of SEQ ID NO: 1, a R at position V93 of SEQ ID NO: 1, and an A at position R97 of SEQ ID NO: 1.

### I16F_P36A_P90I

In some embodiments, a KOD polymerase variant comprises: a F at a position corresponding to I16 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and an I at position P90 of SEQ ID NO: 1. In some embodiments, a KOD polymerase variant comprises: a F at position I16 of SEQ ID NO: 1, an A at position P36 of SEQ ID NO: 1, and an I at position P90 of SEQ ID NO: 1.

### I16F_P36A_P90I_R97A

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, and an A at position R97 of SEQ ID NO: 1.

### I16F_P36A_P90I_R97F

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, and a F at position R97 of SEQ ID NO: 1.

### I16F_P36A_P90I_R97H

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, and a H at position R97 of SEQ ID NO: 1.

### I16F_P36A_P90I_V93I

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, and an I at position V93 of SEQ ID NO: 1.

### I16F_P36A_P90I_V93I_R97A

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1.

### I16F_P36A_P90I_V93I_R97F

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, an I at position V93 of SEQ ID NO: 1, and a F at position R97 of SEQ ID NO: 1.

### I16F_P36A_P90I_V93I_R97H

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, an I at position V93 of SEQ ID NO: 1, and a H at position R97 of SEQ ID NO: 1.

### I16F_P36A_P90I_V93I_R97W

In some embodiments, a KOD polymerase variant comprises: a F at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, an I at position P90 of SEQ ID NO: 1, an I at position V93 of SEQ ID NO: 1, and a W at position R97 of SEQ ID NO: 1.

### I16F_P36A_P90S_V93I

In some embodiments, a KOD polymerase variant comprises: a F at a position corresponding to I16 of SEQ ID NO: 1, an A at a position corresponding to P36 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a S at position P90 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, a S at position P90 of SEQ ID NO: 1, and an I at position V93 of SEQ ID NO: 1.

### I16F_P36A_P90S_V93R_R97A

In some embodiments, a KOD polymerase variant comprises: a F at a position corresponding to I16 of SEQ ID NO: 1, an A at a position corresponding to P36 of SEQ ID NO: 1, a S at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a S at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a S at position P90 of SEQ ID NO: 1, a R at position V93 of SEQ ID NO: 1, and an A at position R97 of SEQ ID NO: 1.

### I16F_P36A_P90W

In some embodiments, a KOD polymerase variant comprises: a F at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a W at position P90 of SEQ ID NO: 1. In some embodiments, a KOD polymerase variant comprises: a F at position I16 of SEQ ID NO: 1, an A at position P36 of SEQ ID NO: 1, and a W at position P90 of SEQ ID NO: 1.

### I16F_P36A_P90W_R97W

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, and a W at position R97 of SEQ ID NO: 1.

### I16F_P36A_P90W_V93I

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, and an I at position V93 of SEQ ID NO: 1.

### I16F_P36A_P90W_V93I_R97A

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### I16F_P36A_P90W_V93I_R97F

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, an I at position V93 of SEQ ID NO: 1, and a F at position R97 of SEQ ID NO: 1.

### I16F_P36A_P90W_V93I_R97H

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1.

### I16F_P36A_P90W_V93I_R97W

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, an I at position V93 of SEQ ID NO: 1, and a W at position R97 of SEQ ID NO: 1.

### I16F_P36A_P90W_V93Q

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, and a Q at position V93 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, and a Q at position V93 of SEQ ID NO: 1.

### I16F_P36A_P90W_V93R_R97A

In some embodiments, a KOD polymerase variant comprises: a F at a position corresponding to I16 of SEQ ID NO: 1, an A at a position corresponding to P36 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a W at position P90 of SEQ ID NO: 1, a R at position V93 of SEQ ID NO: 1, and an A at position R97 of SEQ ID NO: 1.

### I16F_P36A_P90Y

In some embodiments, a KOD polymerase variant comprises: a F at a position corresponding to I16 of SEQ ID NO: 1, an A at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a Y at position P90 of SEQ ID NO: 1. In some embodiments, a KOD polymerase variant comprises: a F at position I16 of SEQ ID NO: 1, an A at position P36 of SEQ ID NO: 1, and a Y at position P90 of SEQ ID NO: 1.

### I16F_P36A_P90Y_R97A

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, and an A at position R97 of SEQ ID NO: 1.

### I16F_P36A_P90Y_R97H

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, and a H at position R97 of SEQ ID NO: 1.

### I16F_P36A_P90Y_V93I

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, and an I at position V93 of SEQ ID NO: 1.

### I16F_P36A_P90Y_V93I_R97A

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### I16F_P36A_P90Y_V93I_R97F

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1.

### I16F_P36A_P90Y_V93I_R97H

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, an I at position V93 of SEQ ID NO: 1, and a H at position R97 of SEQ ID NO: 1.

### I16F_P36A_P90Y_V93I_R97W

In some embodiments, a KOD polymerase variant comprises: a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1.

### I16F_P36A_R97A

In some embodiments, a KOD polymerase variant comprises: a F at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, and an A at position R97 of SEQ ID NO: 1.

### I16F_P36A_R97F

In some embodiments, a KOD polymerase variant comprises: a F at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, and a F at position R97 of SEQ ID NO: 1.

### I16F_P36A_R97H

In some embodiments, a KOD polymerase variant comprises: a F at a position corresponding to I16 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, and a H at position R97 of SEQ ID NO: 1.

### I16F_P36A_R97W

In some embodiments, a KOD polymerase variant comprises: a F at a position corresponding to I16 of SEQ ID NO: 1, an A at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, and a W at position R97 of SEQ ID NO: 1.

### I16F_P36A_V93E

In some embodiments, a KOD polymerase variant comprises: a F at a position corresponding to I16 of SEQ ID NO: 1, an A at a position corresponding to P36 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and an E at position V93 of SEQ ID NO: 1. In some embodiments, a KOD polymerase variant comprises: a F at position I16 of SEQ ID NO: 1, an A at position P36 of SEQ ID NO: 1, and an E at position V93 of SEQ ID NO: 1.

### I16F_P36A_V93I

In some embodiments, a KOD polymerase variant comprises: a F at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and an I at position V93 of SEQ ID NO: 1. In some embodiments, a KOD polymerase variant comprises: a F at position I16 of SEQ ID NO: 1, an A at position P36 of SEQ ID NO: 1, and an I at position V93 of SEQ ID NO: 1.

### I16F_P36A_V93I_R97A

In some embodiments, a KOD polymerase variant comprises: a F at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, an I at position V93 of SEQ ID NO: 1, and an A at position R97 of SEQ ID NO: 1.

### I16F_P36A_V93I_R97F

In some embodiments, a KOD polymerase variant comprises: a F at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, an I at position V93 of SEQ ID NO: 1, and a F at position R97 of SEQ ID NO: 1.

### I16F_P36A_V93I_R97H

In some embodiments, a KOD polymerase variant comprises: a F at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, an I at position V93 of SEQ ID NO: 1, and a H at position R97 of SEQ ID NO: 1.

### I16F_P36A_V93I_R97W

In some embodiments, a KOD polymerase variant comprises: a F at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, an I at position V93 of SEQ ID NO: 1, and a W at position R97 of SEQ ID NO: 1.

### I16F_P36A_V93K

In some embodiments, a KOD polymerase variant comprises: a F at a position corresponding to I16 of SEQ ID NO: 1, an A at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a K at position V93 of SEQ ID NO: 1. In some embodiments, a KOD polymerase variant comprises: a F at position I16 of SEQ ID NO: 1, an A at position P36 of SEQ ID NO: 1, and a K at position V93 of SEQ ID NO: 1.

### I16F_P36A_V93K_R97F

In some embodiments, a KOD polymerase variant comprises: a F at a position corresponding to I16 of SEQ ID NO: 1, an A at a position corresponding to P36 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a K at position V93 of SEQ ID NO: 1, and a F at position R97 of SEQ ID NO: 1.

### I16F_P36A_V93Q

In some embodiments, a KOD polymerase variant comprises: a F at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a Q at position V93 of SEQ ID NO: 1. In some embodiments, a KOD polymerase variant comprises: a F at position I16 of SEQ ID NO: 1, an A at position P36 of SEQ ID NO: 1, and a Q at position V93 of SEQ ID NO: 1.

### I16F_P36A_V93Q_R97A

In some embodiments, a KOD polymerase variant comprises: a F at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a Q at position V93 of SEQ ID NO: 1, and an A at position R97 of SEQ ID NO: 1.

### I16F_P36A_V93Q_R97F

In some embodiments, a KOD polymerase variant comprises: a F at a position corresponding to I16 of SEQ ID NO: 1, an A at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a Q at position V93 of SEQ ID NO: 1, and a F at position R97 of SEQ ID NO: 1.

### I16F_P36A_V93Q_R97W

In some embodiments, a KOD polymerase variant comprises: a F at a position corresponding to I16 of SEQ ID NO: 1, an A at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a Q at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, a Q at position V93 of SEQ ID NO: 1, and a W at position R97 of SEQ ID NO: 1.

### I16F_P36A_V93R

In some embodiments, a KOD polymerase variant comprises: a F at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, and a R at position V93 of SEQ ID NO: 1. In some embodiments, a KOD polymerase variant comprises: a F at position I16 of SEQ ID NO: 1, an A at position P36 of SEQ ID NO: 1, and a R at position V93 of SEQ ID NO: 1.

### I16F_P36A_V93R_R97A

In some embodiments, a KOD polymerase variant comprises: a F at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, an A at position P36 of SEQ ID NO: 1, a R at position V93 of SEQ ID NO: 1, and an A at position R97 of SEQ ID NO: 1.

### I16F_P36A_V93R_R97A_R119Q

In some embodiments, a KOD polymerase variant comprises: a F at a position corresponding to I16 of SEQ ID NO: 1, an A at a position corresponding to P36 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a F at position I16 of SEQ ID NO: 1, an A at position P36 of SEQ ID NO: 1, a R at position V93 of SEQ ID NO: 1, an A at position R97 of SEQ ID NO: 1, and a Q at position R119 of SEQ ID NO: 1.

### I16F_P36A_V93R_R97F

In some embodiments, a KOD polymerase variant comprises: a F at a position corresponding to I16 of SEQ ID NO: 1, an A at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, an A at position P36 of SEQ ID NO: 1, a R at position V93 of SEQ ID NO: 1, and a F at position R97 of SEQ ID NO: 1.

### I16F_P36A_V93R_R97H

In some embodiments, a KOD polymerase variant comprises: a F at a position corresponding to I16 of SEQ ID NO: 1, an A at a position corresponding to P36 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, an A at position P36 of SEQ ID NO: 1, a R at position V93 of SEQ ID NO: 1, and a H at position R97 of SEQ ID NO: 1.

### I16F_P36A_V93R_R97W

In some embodiments, a KOD polymerase variant comprises: a F at a position corresponding to I16 of SEQ ID NO: 1, an A at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position P36 of SEQ ID NO: 1, a R at position V93 of SEQ ID NO: 1, and a W at position R97 of SEQ ID NO: 1. In some embodiments, a KOD polymerase variant comprises: a F at position I16 of SEQ ID NO: 1, an A at position P36 of SEQ ID NO: 1, a R at position V93 of SEQ ID NO: 1, and a W at position R97 of SEQ ID NO: 1.

### I16F_P36H

In some embodiments, a KOD polymerase variant comprises: a F at a position corresponding to I16 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a H at position P36 of SEQ ID NO: 1. In some embodiments, a KOD polymerase variant comprises: a F at position I16 of SEQ ID NO: 1, and a H at position P36 of SEQ ID NO: 1.

### I16F_P36H_P90F

In some embodiments, a KOD polymerase variant comprises: a F at a position corresponding to I16 of SEQ ID NO: 1, a H at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a F at position P90 of SEQ ID NO: 1. In some embodiments, a KOD polymerase variant comprises: a F at position I16 of SEQ ID NO: 1, a H at position P36 of SEQ ID NO: 1, and a F at position P90 of SEQ ID NO: 1.

### I16F_P36H_P90F_V93I

In some embodiments, a KOD polymerase variant comprises: a F at a position corresponding to I16 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a F at position I16 of SEQ ID NO: 1, a H at position P36 of SEQ ID NO: 1, a F at position P90 of SEQ ID NO: 1, and an I at position V93 of SEQ ID NO: 1.

### I16F_P36H_P90W

In some embodiments, a KOD polymerase variant comprises: a F at a position corresponding to I16 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a W at position P90 of SEQ ID NO: 1. In some embodiments, a KOD polymerase variant comprises: a F at position I16 of SEQ ID NO: 1, a H at position P36 of SEQ ID NO: 1, and a W at position P90 of SEQ ID NO: 1.

### I16F_P36H_P90W_R97H

In some embodiments, a KOD polymerase variant comprises: a F at a position corresponding to I16 of SEQ ID NO: 1, a H at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, a W at position P90 of SEQ ID NO: 1, and a H at position R97 of SEQ ID NO: 1. In some embodiments, a KOD polymerase variant comprises: a F at position I16 of SEQ ID NO: 1, a H at position P36 of SEQ ID NO: 1, a W at position P90 of SEQ ID NO: 1, and a H at position R97 of SEQ ID NO: 1.

### I16F_P36H_P90Y_R97A

In some embodiments, a KOD polymerase variant comprises: a F at a position corresponding to I16 of SEQ ID NO: 1, a H at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, a Y at position P90 of SEQ ID NO: 1, and an A at position R97 of SEQ ID NO: 1. In some embodiments, a KOD polymerase variant comprises: a F at position I16 of SEQ ID NO: 1, a H at position P36 of SEQ ID NO: 1, a Y at position P90 of SEQ ID NO: 1, and an A at position R97 of SEQ ID NO: 1.

### I16F_P36H_R97A

In some embodiments, a KOD polymerase variant comprises: a F at a position corresponding to I16 of SEQ ID NO: 1, a H at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and an A at position R97 of SEQ ID NO: 1. In some embodiments, a KOD polymerase variant comprises: a F at position I16 of SEQ ID NO: 1, a H at position P36 of SEQ ID NO: 1, and an A at position R97 of SEQ ID NO: 1.

### I16F_P36H_R97F

In some embodiments, a KOD polymerase variant comprises: a F at a position corresponding to I16 of SEQ ID NO: 1, a H at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a F at position R97 of SEQ ID NO: 1. In some embodiments, a KOD polymerase variant comprises: a F at position I16 of SEQ ID NO: 1, a H at position P36 of SEQ ID NO: 1, and a F at position R97 of SEQ ID NO: 1.

### I16F_P36H_R97H

In some embodiments, a KOD polymerase variant comprises: a F at a position corresponding to I16 of SEQ ID NO: 1, a H at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a H at position R97 of SEQ ID NO: 1. In some embodiments, a KOD polymerase variant comprises: a F at position I16 of SEQ ID NO: 1, a H at position P36 of SEQ ID NO: 1, and a H at position R97 of SEQ ID NO: 1.

### I16F_P36H_R119Q

In some embodiments, a KOD polymerase variant comprises: a F at a position corresponding to I16 of SEQ ID NO: 1, a H at a position corresponding to P36 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a Q at position R119 of SEQ ID NO: 1. In some embodiments, a KOD polymerase variant comprises: a F at position I16 of SEQ ID NO: 1, a H at position P36 of SEQ ID NO: 1, and a Q at position R119 of SEQ ID NO: 1.

### I16F_P36H_V93I

In some embodiments, a KOD polymerase variant comprises: a F at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and an I at position V93 of SEQ ID NO: 1. In some embodiments, a KOD polymerase variant comprises: a F at position I16 of SEQ ID NO: 1, a H at position P36 of SEQ ID NO: 1, and an I at position V93 of SEQ ID NO: 1.

### I16F_P36H_V93I_R97A

In some embodiments, a KOD polymerase variant comprises: a F at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a H at position P36 of SEQ ID NO: 1, an I at position V93 of SEQ ID NO: 1, and an A at position R97 of SEQ ID NO: 1.

### I16F_P36H_V93I_R97F

In some embodiments, a KOD polymerase variant comprises: a F at a position corresponding to I16 of SEQ ID NO: 1, a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a H at position P36 of SEQ ID NO: 1, an I at position V93 of SEQ ID NO: 1, and a F at position R97 of SEQ ID NO: 1.

### I16F_P36H_V93I_R97H

In some embodiments, a KOD polymerase variant comprises: a F at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, a H at position P36 of SEQ ID NO: 1, an I at position V93 of SEQ ID NO: 1, and a H at position R97 of SEQ ID NO: 1.

### I16F_P36H_V93Q

In some embodiments, a KOD polymerase variant comprises: a F at a position corresponding to I16 of SEQ ID NO: 1, a H at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a H at position P36 of SEQ ID NO: 1, and a Q at position V93 of SEQ ID NO: 1. In some embodiments, a KOD polymerase variant comprises: a F at position I16 of SEQ ID NO: 1, a H at position P36 of SEQ ID NO: 1, and a Q at position V93 of SEQ ID NO: 1.

### I16F_P36R

In some embodiments, a KOD polymerase variant comprises: a F at a position corresponding to I16 of SEQ ID NO: 1 and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a R at position P36 of SEQ ID NO: 1. In some embodiments, a KOD polymerase variant comprises: a F at position I16 of SEQ ID NO: 1, and a R at position P36 of SEQ ID NO: 1.

### I16F_P36R_R97A

In some embodiments, a KOD polymerase variant comprises: a F at a position corresponding to I16 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position P36 of SEQ ID NO: 1, and an A at position R97 of SEQ ID NO: 1. In some embodiments, a KOD polymerase variant comprises: a F at position I16 of SEQ ID NO: 1, a R at position P36 of SEQ ID NO: 1, and an A at position R97 of SEQ ID NO: 1.

### I16F_P36R_R97F

In some embodiments, a KOD polymerase variant comprises: a F at a position corresponding to I16 of SEQ ID NO: 1, a R at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position P36 of SEQ ID NO: 1, and a F at position R97 of SEQ ID NO: 1. In some embodiments, a KOD polymerase variant comprises: a F at position I16 of SEQ ID NO: 1, a R at position P36 of SEQ ID NO: 1, and a F at position R97 of SEQ ID NO: 1.

### I16F_P36R_R97H

In some embodiments, a KOD polymerase variant comprises: a F at a position corresponding to I16 of SEQ ID NO: 1, a R at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position P36 of SEQ ID NO: 1, and a H at position R97 of SEQ ID NO: 1. In some embodiments, a KOD polymerase variant comprises: a F at position I16 of SEQ ID NO: 1, a R at position P36 of SEQ ID NO: 1, and a H at position R97 of SEQ ID NO: 1.

### I16F_P36S

In some embodiments, a KOD polymerase variant comprises: a F at a position corresponding to I16 of SEQ ID NO: 1 and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a S at position P36 of SEQ ID NO: 1. In some embodiments, a KOD polymerase variant comprises: a F at position I16 of SEQ ID NO: 1, and a S at position P36 of SEQ ID NO: 1.

### I16F_P36S_P90F_R97A

In some embodiments, a KOD polymerase variant comprises: a F at a position corresponding to I16 of SEQ ID NO: 1, a S at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a F at position P90 of SEQ ID NO: 1, and an A at position R97 of SEQ ID NO: 1. In some embodiments, a KOD polymerase variant comprises: a F at position I16 of SEQ ID NO: 1, a S at position P36 of SEQ ID NO: 1, a F at position P90 of SEQ ID NO: 1, and an A at position R97 of SEQ ID NO: 1.

### I16F_P36S_P90F_V93I

In some embodiments, a KOD polymerase variant comprises: a F at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a F at position I16 of SEQ ID NO: 1, a S at position P36 of SEQ ID NO: 1, a F at position P90 of SEQ ID NO: 1, and an I at position V93 of SEQ ID NO: 1.

### I16F_P36S_P90F_V93I_R97A

In some embodiments, a KOD polymerase variant comprises: a F at a position corresponding to I16 of SEQ ID NO: 1, a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1.

### I16F_P36S_P90I_V93I

In some embodiments, a KOD polymerase variant comprises: a F at a position corresponding to I16 of SEQ ID NO: 1, a S at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, an I at position P90 of SEQ ID NO: 1, and an I at position V93 of SEQ ID NO: 1. In some embodiments, a KOD polymerase variant comprises: a F at position I16 of SEQ ID NO: 1, a S at position P36 of SEQ ID NO: 1, an I at position P90 of SEQ ID NO: 1, and an I at position V93 of SEQ ID NO: 1.

### I16F_P36S_P90I_V93I_R97F

In some embodiments, a KOD polymerase variant comprises: a F at a position corresponding to I16 of SEQ ID NO: 1, a S at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a S at position P36 of SEQ ID NO: 1, an I at position P90 of SEQ ID NO: 1, an I at position V93 of SEQ ID NO: 1, and a F at position R97 of SEQ ID NO: 1.

### I16F_P36S_P90W_R97A

In some embodiments, a KOD polymerase variant comprises: a F at a position corresponding to I16 of SEQ ID NO: 1, a S at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a W at position P90 of SEQ ID NO: 1, and an A at position R97 of SEQ ID NO: 1. In some embodiments, a KOD polymerase variant comprises: a F at position I16 of SEQ ID NO: 1, a S at position P36 of SEQ ID NO: 1, a W at position P90 of SEQ ID NO: 1, and an A at position R97 of SEQ ID NO: 1.

### I16F_P36S_P90Y

In some embodiments, a KOD polymerase variant comprises: a F at a position corresponding to I16 of SEQ ID NO: 1, a S at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and a Y at position P90 of SEQ ID NO: 1. In some embodiments, a KOD polymerase variant comprises: a F at position I16 of SEQ ID NO: 1, a S at position P36 of SEQ ID NO: 1, and a Y at position P90 of SEQ ID NO: 1.

### I16F_P36S_R97A

In some embodiments, a KOD polymerase variant comprises: a F at a position corresponding to I16 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and an A at position R97 of SEQ ID NO: 1. In some embodiments, a KOD polymerase variant comprises: a F at position I16 of SEQ ID NO: 1, a S at position P36 of SEQ ID NO: 1, and an A at position R97 of SEQ ID NO: 1.

### I16F_P36S_R97F

In some embodiments, a KOD polymerase variant comprises: a F at a position corresponding to I16 of SEQ ID NO: 1, a S at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and a F at position R97 of SEQ ID NO: 1. In some embodiments, a KOD polymerase variant comprises: a F at position I16 of SEQ ID NO: 1, a S at position P36 of SEQ ID NO: 1, and a F at position R97 of SEQ ID NO: 1.

### I16F_P36S_R97H

In some embodiments, a KOD polymerase variant comprises: a F at a position corresponding to I16 of SEQ ID NO: 1, a S at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and a H at position R97 of SEQ ID NO: 1. In some embodiments, a KOD polymerase variant comprises: a F at position I16 of SEQ ID NO: 1, a S at position P36 of SEQ ID NO: 1, and a H at position R97 of SEQ ID NO: 1.

### I16F_P36S_V93I

In some embodiments, a KOD polymerase variant comprises: a F at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and an I at position V93 of SEQ ID NO: 1. In some embodiments, a KOD polymerase variant comprises: a F at position I16 of SEQ ID NO: 1, a S at position P36 of SEQ ID NO: 1, and an I at position V93 of SEQ ID NO: 1.

### I16F_P36S_V93I_R97F

In some embodiments, a KOD polymerase variant comprises: a F at a position corresponding to I16 of SEQ ID NO: 1, a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a S at position P36 of SEQ ID NO: 1, an I at position V93 of SEQ ID NO: 1, and a F at position R97 of SEQ ID NO: 1.

### I16F_P36S_V93I_R97W

In some embodiments, a KOD polymerase variant comprises: a F at a position corresponding to I16 of SEQ ID NO: 1, a S at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, a S at position P36 of SEQ ID NO: 1, an I at position V93 of SEQ ID NO: 1, and a W at position R97 of SEQ ID NO: 1.

### I16F_P36S_V93Q

In some embodiments, a KOD polymerase variant comprises: a F at a position corresponding to I16 of SEQ ID NO: 1, a S at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, and a Q at position V93 of SEQ ID NO: 1. In some embodiments, a KOD polymerase variant comprises: a F at position I16 of SEQ ID NO: 1, a S at position P36 of SEQ ID NO: 1, and a Q at position V93 of SEQ ID NO: 1.

### I16F_P36S_V93R_R97F

In some embodiments, a KOD polymerase variant comprises: a F at a position corresponding to I16 of SEQ ID NO: 1, a S at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a S at position P36 of SEQ ID NO: 1, a R at position V93 of SEQ ID NO: 1, and a F at position R97 of SEQ ID NO: 1.

### I16F_P36S_V93R_R97H

In some embodiments, a KOD polymerase variant comprises: a F at a position corresponding to I16 of SEQ ID NO: 1, a S at a position corresponding to P36 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P36 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, a S at position P36 of SEQ ID NO: 1, a R at position V93 of SEQ ID NO: 1, and a H at position R97 of SEQ ID NO: 1.

### I16F_P36W

In some embodiments, a KOD polymerase variant comprises: a F at a position corresponding to I16 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a W at position P36 of SEQ ID NO: 1. In some embodiments, a KOD polymerase variant comprises: a F at position I16 of SEQ ID NO: 1, and a W at position P36 of SEQ ID NO: 1.

### I16F_P36W_P90F_V93I

In some embodiments, a KOD polymerase variant comprises: a F at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a F at position I16 of SEQ ID NO: 1, a W at position P36 of SEQ ID NO: 1, a F at position P90 of SEQ ID NO: 1, and an I at position V93 of SEQ ID NO: 1.

### I16F_P36W_P90F_V93I_R97A

In some embodiments, a KOD polymerase variant comprises: a F at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### I16F_P36W_P90F_V93I_R97F

In some embodiments, a KOD polymerase variant comprises: a F at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### I16F_P36W_P90F_V93K_R97A

In some embodiments, a KOD polymerase variant comprises: a F at a position corresponding to I16 of SEQ ID NO: 1, a W at a position corresponding to P36 of SEQ ID NO: 1, a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a F at position P90 of SEQ ID NO: 1, a K at position V93 of SEQ ID NO: 1, and an A at position R97 of SEQ ID NO: 1. In some embodiments, a KOD polymerase variant comprises: a F at position I16 of SEQ ID NO: 1, a W at position P36 of SEQ ID NO: 1, a F at position P90 of SEQ ID NO: 1, a K at position V93 of SEQ ID NO: 1, and an A at position R97 of SEQ ID NO: 1.

### I16F_P36W_P90F_V93R_R97A

In some embodiments, a KOD polymerase variant comprises: a F at a position corresponding to I16 of SEQ ID NO: 1, a W at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1.

### I16F_P36W_P90I_V93I

In some embodiments, a KOD polymerase variant comprises: a F at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a F at position I16 of SEQ ID NO: 1, a W at position P36 of SEQ ID NO: 1, an I at position P90 of SEQ ID NO: 1, and an I at position V93 of SEQ ID NO: 1.

### I16F_P36W_P90I_V93R_R97F

In some embodiments, a KOD polymerase variant comprises: a F at a position corresponding to I16 of SEQ ID NO: 1, a W at a position corresponding to P36 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a W at position P36 of SEQ ID NO: 1, an I at position P90 of SEQ ID NO: 1, a R at position V93 of SEQ ID NO: 1, and a F at position R97 of SEQ ID NO: 1.

### I16F_P36W_P90W_V93I

In some embodiments, a KOD polymerase variant comprises: a F at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a F at position I16 of SEQ ID NO: 1, a W at position P36 of SEQ ID NO: 1, a W at position P90 of SEQ ID NO: 1, and an I at position V93 of SEQ ID NO: 1.

### I16F_P36W_P90Y_V93K_R97F

In some embodiments, a KOD polymerase variant comprises: a F at a position corresponding to I16 of SEQ ID NO: 1, a W at a position corresponding to P36 of SEQ ID NO: 1, a Y at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, a Y at position P90 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, a W at position P36 of SEQ ID NO: 1, a Y at position P90 of SEQ ID NO: 1, a K at position V93 of SEQ ID NO: 1, and a F at position R97 of SEQ ID NO: 1.

### I16F_P36W_R97A

In some embodiments, a KOD polymerase variant comprises: a F at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, and an A at position R97 of SEQ ID NO: 1. In some embodiments, a KOD polymerase variant comprises: a F at position I16 of SEQ ID NO: 1, a W at position P36 of SEQ ID NO: 1, and an A at position R97 of SEQ ID NO: 1.

### I16F_P36W_R97F

In some embodiments, a KOD polymerase variant comprises: a F at a position corresponding to I16 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, and a F at position R97 of SEQ ID NO: 1. In some embodiments, a KOD polymerase variant comprises: a F at position I16 of SEQ ID NO: 1, a W at position P36 of SEQ ID NO: 1, and a F at position R97 of SEQ ID NO: 1.

### I16F_P36W_V93I

In some embodiments, a KOD polymerase variant comprises: a F at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, and an I at position V93 of SEQ ID NO: 1. In some embodiments, a KOD polymerase variant comprises: a F at position I16 of SEQ ID NO: 1, a W at position P36 of SEQ ID NO: 1, and an I at position V93 of SEQ ID NO: 1.

### I16F_P36W_V93I_R97A

In some embodiments, a KOD polymerase variant comprises: a F at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a W at position P36 of SEQ ID NO: 1, an I at position V93 of SEQ ID NO: 1, and an A at position R97 of SEQ ID NO: 1.

### I16F_P90F

In some embodiments, a KOD polymerase variant comprises: a F at a position corresponding to I16 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a F at position P90 of SEQ ID NO: 1. In some embodiments, a KOD polymerase variant comprises: a F at position I16 of SEQ ID NO: 1, and a F at position P90 of SEQ ID NO: 1.

### I16F_P90F_R97A

In some embodiments, a KOD polymerase variant comprises: a F at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and an A at position R97 of SEQ ID NO: 1. In some embodiments, a KOD polymerase variant comprises: a F at position I16 of SEQ ID NO: 1, a F at position P90 of SEQ ID NO: 1, and an A at position R97 of SEQ ID NO: 1.

### I16F_P90F_R97F

In some embodiments, a KOD polymerase variant comprises: a F at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a F at position R97 of SEQ ID NO: 1. In some embodiments, a KOD polymerase variant comprises: a F at position I16 of SEQ ID NO: 1, a F at position P90 of SEQ ID NO: 1, and a F at position R97 of SEQ ID NO: 1.

### I16F_P90F_R97H

In some embodiments, a KOD polymerase variant comprises: a F at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a H at position R97 of SEQ ID NO: 1. In some embodiments, a KOD polymerase variant comprises: a F at position I16 of SEQ ID NO: 1, a F at position P90 of SEQ ID NO: 1, and a H at position R97 of SEQ ID NO: 1.

### I16F_P90F_R97W

In some embodiments, a KOD polymerase variant comprises: a F at a position corresponding to I16 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a W at position R97 of SEQ ID NO: 1. In some embodiments, a KOD polymerase variant comprises: a F at position I16 of SEQ ID NO: 1, a F at position P90 of SEQ ID NO: 1, and a W at position R97 of SEQ ID NO: 1.

### I16F_P90F_V93I

In some embodiments, a KOD polymerase variant comprises: a F at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and an I at position V93 of SEQ ID NO: 1. In some embodiments, a KOD polymerase variant comprises: a F at position I16 of SEQ ID NO: 1, a F at position P90 of SEQ ID NO: 1, and an I at position V93 of SEQ ID NO: 1.

### I16F_P90F_V93I_R97A

In some embodiments, a KOD polymerase variant comprises: a F at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a F at position P90 of SEQ ID NO: 1, an I at position V93 of SEQ ID NO: 1, and an A at position R97 of SEQ ID NO: 1.

### I16F_P90F_V93I_R97F

In some embodiments, a KOD polymerase variant comprises: a F at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a F at position P90 of SEQ ID NO: 1, an I at position V93 of SEQ ID NO: 1, and a F at position R97 of SEQ ID NO: 1.

### I16F_P90F_V93I_R97H

In some embodiments, a KOD polymerase variant comprises: a F at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, a F at position P90 of SEQ ID NO: 1, an I at position V93 of SEQ ID NO: 1, and a H at position R97 of SEQ ID NO: 1.

### I16F_P90F_V93I_R97W

In some embodiments, a KOD polymerase variant comprises: a F at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, a F at position P90 of SEQ ID NO: 1, an I at position V93 of SEQ ID NO: 1, and a W at position R97 of SEQ ID NO: 1.

### I16F_P90F_V93K

In some embodiments, a KOD polymerase variant comprises: a F at a position corresponding to I16 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a K at position V93 of SEQ ID NO: 1. In some embodiments, a KOD polymerase variant comprises: a F at position I16 of SEQ ID NO: 1, a F at position P90 of SEQ ID NO: 1, and a K at position V93 of SEQ ID NO: 1.

### I16F_P90F_V93K_R97A

In some embodiments, a KOD polymerase variant comprises: a F at a position corresponding to I16 of SEQ ID NO: 1, a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a K at position V93 of SEQ ID NO: 1, and an A at position R97 of SEQ ID NO: 1. In some embodiments, a KOD polymerase variant comprises: a F at position I16 of SEQ ID NO: 1, a F at position P90 of SEQ ID NO: 1, a K at position V93 of SEQ ID NO: 1, and an A at position R97 of SEQ ID NO: 1.

### I16F_P90F_V93K_R97F

In some embodiments, a KOD polymerase variant comprises: a F at a position corresponding to I16 of SEQ ID NO: 1, a F at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, a F at position P90 of SEQ ID NO: 1, a K at position V93 of SEQ ID NO: 1, and a F at position R97 of SEQ ID NO: 1.

### I16F_P90F_V93K_R97H

In some embodiments, a KOD polymerase variant comprises: a F at a position corresponding to I16 of SEQ ID NO: 1, a F at a position corresponding to P90 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a K at position V93 of SEQ ID NO: 1, and a H at position R97 of SEQ ID NO: 1. In some embodiments, a KOD polymerase variant comprises: a F at position I16 of SEQ ID NO: 1, a F at position P90 of SEQ ID NO: 1, a K at position V93 of SEQ ID NO: 1, and a H at position R97 of SEQ ID NO: 1.

### I16F_P90F_V93Q

In some embodiments, a KOD polymerase variant comprises: a F at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a Q at position V93 of SEQ ID NO: 1. In some embodiments, a KOD polymerase variant comprises: a F at position I16 of SEQ ID NO: 1, a F at position P90 of SEQ ID NO: 1, and a Q at position V93 of SEQ ID NO: 1.

### I16F_P90F_V93Q_R97A

In some embodiments, a KOD polymerase variant comprises: a F at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, a F at position P90 of SEQ ID NO: 1, a Q at position V93 of SEQ ID NO: 1, and an A at position R97 of SEQ ID NO: 1.

### I16F_P90F_V93Q_R97F

In some embodiments, a KOD polymerase variant comprises: a F at a position corresponding to I16 of SEQ ID NO: 1, a F at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, a F at position P90 of SEQ ID NO: 1, a Q at position V93 of SEQ ID NO: 1, and a F at position R97 of SEQ ID NO: 1.

### I16F_P90F_V93Q_R97H

In some embodiments, a KOD polymerase variant comprises: a F at a position corresponding to I16 of SEQ ID NO: 1, a F at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a Q at position V93 of SEQ ID NO: 1, and a H at position R97 of SEQ ID NO: 1. In some embodiments, a KOD polymerase variant comprises: a F at position I16 of SEQ ID NO: 1, a F at position P90 of SEQ ID NO: 1, a Q at position V93 of SEQ ID NO: 1, and a H at position R97 of SEQ ID NO: 1.

### I16F_P90F_V93Q_R97W

In some embodiments, a KOD polymerase variant comprises: a F at a position corresponding to I16 of SEQ ID NO: 1, a F at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a Q at position V93 of SEQ ID NO: 1, and a W at position R97 of SEQ ID NO: 1. In some embodiments, a KOD polymerase variant comprises: a F at position I16 of SEQ ID NO: 1, a F at position P90 of SEQ ID NO: 1, a Q at position V93 of SEQ ID NO: 1, and a W at position R97 of SEQ ID NO: 1.

### I16F_P90F_V93R

In some embodiments, a KOD polymerase variant comprises: a F at a position corresponding to I16 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, and a R at position V93 of SEQ ID NO: 1. In some embodiments, a KOD polymerase variant comprises: a F at position I16 of SEQ ID NO: 1, a F at position P90 of SEQ ID NO: 1, and a R at position V93 of SEQ ID NO: 1.

### I16F_P90F_V93R_R97A

In some embodiments, a KOD polymerase variant comprises: a F at a position corresponding to I16 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, a F at position P90 of SEQ ID NO: 1, a R at position V93 of SEQ ID NO: 1, and an A at position R97 of SEQ ID NO: 1.

### I16F_P90F_V93R_R97F

In some embodiments, a KOD polymerase variant comprises: a F at a position corresponding to I16 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a F at position P90 of SEQ ID NO: 1, a R at position V93 of SEQ ID NO: 1, and a F at position R97 of SEQ ID NO: 1.

### I16F_P90F_V93R_R97H

In some embodiments, a KOD polymerase variant comprises: a F at a position corresponding to I16 of SEQ ID NO: 1, a F at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, a F at position P90 of SEQ ID NO: 1, a R at position V93 of SEQ ID NO: 1, and a H at position R97 of SEQ ID NO: 1.

### I16F_P90F_V93R_R97P

In some embodiments, a KOD polymerase variant comprises: a F at a position corresponding to I16 of SEQ ID NO: 1, a F at a position corresponding to P90 of SEQ ID NO: 1, a R at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position P90 of SEQ ID NO: 1, a R at position V93 of SEQ ID NO: 1, and a P at position R97 of SEQ ID NO: 1. In some embodiments, a KOD polymerase variant comprises: a F at position I16 of SEQ ID NO: 1, a F at position P90 of SEQ ID NO: 1, a R at position V93 of SEQ ID NO: 1, and a P at position R97 of SEQ ID NO: 1.

### I16F_P90I

In some embodiments, a KOD polymerase variant comprises: a F at a position corresponding to I16 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and an I at position P90 of SEQ ID NO: 1. In some embodiments, a KOD polymerase variant comprises: a F at position I16 of SEQ ID NO: 1, and an I at position P90 of SEQ ID NO: 1.

### I16F_P90I_R97A

In some embodiments, a KOD polymerase variant comprises: a F at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and an A at position R97 of SEQ ID NO: 1. In some embodiments, a KOD polymerase variant comprises: a F at position I16 of SEQ ID NO: 1, an I at position P90 of SEQ ID NO: 1, and an A at position R97 of SEQ ID NO: 1.

### I16F_P90I_R97F

In some embodiments, a KOD polymerase variant comprises: a F at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a F at position R97 of SEQ ID NO: 1. In some embodiments, a KOD polymerase variant comprises: a F at position I16 of SEQ ID NO: 1, an I at position P90 of SEQ ID NO: 1, and a F at position R97 of SEQ ID NO: 1.

### I16F_P90I_R97H

In some embodiments, a KOD polymerase variant comprises: a F at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a H at position R97 of SEQ ID NO: 1. In some embodiments, a KOD polymerase variant comprises: a F at position I16 of SEQ ID NO: 1, an I at position P90 of SEQ ID NO: 1, and a H at position R97 of SEQ ID NO: 1.

### I16F_P90I_R97P

In some embodiments, a KOD polymerase variant comprises: a F at a position corresponding to I16 of SEQ ID NO: 1, an I at a position corresponding to P90 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a P at position R97 of SEQ ID NO: 1. In some embodiments, a KOD polymerase variant comprises: a F at position I16 of SEQ ID NO: 1, an I at position P90 of SEQ ID NO: 1, and a P at position R97 of SEQ ID NO: 1.

### I16F_P90I_R97W

In some embodiments, a KOD polymerase variant comprises: a F at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a W at position R97 of SEQ ID NO: 1. In some embodiments, a KOD polymerase variant comprises: a F at position I16 of SEQ ID NO: 1, an I at position P90 of SEQ ID NO: 1, and a W at position R97 of SEQ ID NO: 1.

### I16F_P90I_V93E

In some embodiments, a KOD polymerase variant comprises: a F at a position corresponding to I16 of SEQ ID NO: 1, an I at a position corresponding to P90 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and an E at position V93 of SEQ ID NO: 1. In some embodiments, a KOD polymerase variant comprises: a F at position I16 of SEQ ID NO: 1, an I at position P90 of SEQ ID NO: 1, and an E at position V93 of SEQ ID NO: 1.

### I16F_P90I_V93I

In some embodiments, a KOD polymerase variant comprises: a F at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and an I at position V93 of SEQ ID NO: 1. In some embodiments, a KOD polymerase variant comprises: a F at position I16 of SEQ ID NO: 1, an I at position P90 of SEQ ID NO: 1, and an I at position V93 of SEQ ID NO: 1.

### I16F_P90I_V93I_R97A

In some embodiments, a KOD polymerase variant comprises: a F at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an I at position P90 of SEQ ID NO: 1, an I at position V93 of SEQ ID NO: 1, and an A at position R97 of SEQ ID NO: 1.

### I16F_P90I_V93I_R97F

In some embodiments, a KOD polymerase variant comprises: a F at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an I at position P90 of SEQ ID NO: 1, an I at position V93 of SEQ ID NO: 1, and a F at position R97 of SEQ ID NO: 1.

### I16F_P90I_V93I_R97H

In some embodiments, a KOD polymerase variant comprises: a F at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an I at position P90 of SEQ ID NO: 1, an I at position V93 of SEQ ID NO: 1, and a H at position R97 of SEQ ID NO: 1.

### I16F_P90I_V93I_R97P

In some embodiments, a KOD polymerase variant comprises: a F at a position corresponding to I16 of SEQ ID NO: 1, an I at a position corresponding to P90 of SEQ ID NO: 1, an I at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, an I at position V93 of SEQ ID NO: 1, and a P at position R97 of SEQ ID NO: 1. In some embodiments, a KOD polymerase variant comprises: a F at position I16 of SEQ ID NO: 1, an I at position P90 of SEQ ID NO: 1, an I at position V93 of SEQ ID NO: 1, and a P at position R97 of SEQ ID NO: 1.

### I16F_P90I_V93I_R97W

In some embodiments, a KOD polymerase variant comprises: a F at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an I at position P90 of SEQ ID NO: 1, an I at position V93 of SEQ ID NO: 1, and a W at position R97 of SEQ ID NO: 1.

### I16F_P90I_V93K

In some embodiments, a KOD polymerase variant comprises: a F at a position corresponding to I16 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a K at position V93 of SEQ ID NO: 1. In some embodiments, a KOD polymerase variant comprises: a F at position I16 of SEQ ID NO: 1, an I at position P90 of SEQ ID NO: 1, and a K at position V93 of SEQ ID NO: 1.

### I16F_P90I_V93K_R97A

In some embodiments, a KOD polymerase variant comprises: a F at a position corresponding to I16 of SEQ ID NO: 1, an I at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a K at position V93 of SEQ ID NO: 1, and an A at position R97 of SEQ ID NO: 1. In some embodiments, a KOD polymerase variant comprises: a F at position I16 of SEQ ID NO: 1, an I at position P90 of SEQ ID NO: 1, a K at position V93 of SEQ ID NO: 1, and an A at position R97 of SEQ ID NO: 1.

### I16F_P90I_V93K_R97F

In some embodiments, a KOD polymerase variant comprises: a F at a position corresponding to I16 of SEQ ID NO: 1, an I at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, an I at position P90 of SEQ ID NO: 1, a K at position V93 of SEQ ID NO: 1, and a F at position R97 of SEQ ID NO: 1.

### I16F_P90I_V93K_R97W

In some embodiments, a KOD polymerase variant comprises: a F at a position corresponding to I16 of SEQ ID NO: 1, an I at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a K at position V93 of SEQ ID NO: 1, and a W at position R97 of SEQ ID NO: 1. In some embodiments, a KOD polymerase variant comprises: a F at position I16 of SEQ ID NO: 1, an I at position P90 of SEQ ID NO: 1, a K at position V93 of SEQ ID NO: 1, and a W at position R97 of SEQ ID NO:1.

### I16F_P90I_V93Q

In some embodiments, a KOD polymerase variant comprises: a F at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a Q at position V93 of SEQ ID NO: 1. In some embodiments, a KOD polymerase variant comprises: a F at position I16 of SEQ ID NO: 1, an I at position P90 of SEQ ID NO: 1, and a Q at position V93 of SEQ ID NO: 1.

### I16F_P90I_V93Q_R97A

In some embodiments, a KOD polymerase variant comprises: a F at a position corresponding to I16 of SEQ ID NO: 1, an I at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, an I at position P90 of SEQ ID NO: 1, a Q at position V93 of SEQ ID NO: 1, and an A at position R97 of SEQ ID NO: 1.

### I16F_P90I_V93Q_R97F

In some embodiments, a KOD polymerase variant comprises: a F at a position corresponding to I16 of SEQ ID NO: 1, an I at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, an I at position P90 of SEQ ID NO: 1, a Q at position V93 of SEQ ID NO: 1, and a F at position R97 of SEQ ID NO: 1.

### I16F_P90I_V93Q_R97H

In some embodiments, a KOD polymerase variant comprises: a F at a position corresponding to I16 of SEQ ID NO: 1, an I at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a Q at position V93 of SEQ ID NO: 1, and a H at position R97 of SEQ ID NO: 1. In some embodiments, a KOD polymerase variant comprises: a F at position I16 of SEQ ID NO: 1, an I at position P90 of SEQ ID NO: 1, a Q at position V93 of SEQ ID NO: 1, and a H at position R97 of SEQ ID NO: 1.

### I16F_P90I_V93R

In some embodiments, a KOD polymerase variant comprises: a F at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, and a R at position V93 of SEQ ID NO: 1. In some embodiments, a KOD polymerase variant comprises: a F at position I16 of SEQ ID NO: 1, an I at position P90 of SEQ ID NO: 1, and a R at position V93 of SEQ ID NO: 1.

### I16F_P90I_V93R_R97A

In some embodiments, a KOD polymerase variant comprises: a F at a position corresponding to I16 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, an I at position P90 of SEQ ID NO: 1, a R at position V93 of SEQ ID NO: 1, and an A at position R97 of SEQ ID NO: 1.

### I16F_P90I_V93R_R97A_R119Q

In some embodiments, a KOD polymerase variant comprises: a F at a position corresponding to I16 of SEQ ID NO: 1, an I at a position corresponding to P90 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a F at position I16 of SEQ ID NO: 1, an I at position P90 of SEQ ID NO: 1, a R at position V93 of SEQ ID NO: 1, an A at position R97 of SEQ ID NO: 1, and a Q at position R119 of SEQ ID NO: 1.

### I16F_P90I_V93R_R97F

In some embodiments, a KOD polymerase variant comprises: a F at a position corresponding to I16 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, an I at position P90 of SEQ ID NO: 1, a R at position V93 of SEQ ID NO: 1, and a F at position R97 of SEQ ID NO: 1.

### I16F_P90I_V93R_R97H

In some embodiments, a KOD polymerase variant comprises: a F at a position corresponding to I16 of SEQ ID NO: 1, an I at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, an I at position P90 of SEQ ID NO: 1, a R at position V93 of SEQ ID NO: 1, and a H at position R97 of SEQ ID NO: 1.

### I16F_P90I_V93R_R97W

In some embodiments, a KOD polymerase variant comprises: a F at a position corresponding to I16 of SEQ ID NO: 1, an I at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position P90 of SEQ ID NO: 1, a R at position V93 of SEQ ID NO: 1, and a W at position R97 of SEQ ID NO: 1. In some embodiments, a KOD polymerase variant comprises: a F at position I16 of SEQ ID NO: 1, an I at position P90 of SEQ ID NO: 1, a R at position V93 of SEQ ID NO: 1, and a W at position R97 of SEQ ID NO: 1.

### I16F_P90S

In some embodiments, a KOD polymerase variant comprises: a F at a position corresponding to I16 of SEQ ID NO: 1 and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a S at position P90 of SEQ ID NO: 1. In some embodiments, a KOD polymerase variant comprises: a F at position I16 of SEQ ID NO: 1, and a S at position P90 of SEQ ID NO: 1.

### I16F_P90S_R97A

In some embodiments, a KOD polymerase variant comprises: a F at a position corresponding to I16 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and an A at position R97 of SEQ ID NO: 1. In some embodiments, a KOD polymerase variant comprises: a F at position I16 of SEQ ID NO: 1, a S at position P90 of SEQ ID NO: 1, and an A at position R97 of SEQ ID NO: 1.

### I16F_P90S_R97F

In some embodiments, a KOD polymerase variant comprises: a F at a position corresponding to I16 of SEQ ID NO: 1, a S at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and a F at position R97 of SEQ ID NO: 1. In some embodiments, a KOD polymerase variant comprises: a F at position I16 of SEQ ID NO: 1, a S at position P90 of SEQ ID NO: 1, and a F at position R97 of SEQ ID NO: 1.

### I16F_P90S_R97W

In some embodiments, a KOD polymerase variant comprises: a F at a position corresponding to I16 of SEQ ID NO: 1, a S at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and a W at position R97 of SEQ ID NO: 1. In some embodiments, a KOD polymerase variant comprises: a F at position I16 of SEQ ID NO: 1, a S at position P90 of SEQ ID NO: 1, and a W at position R97 of SEQ ID NO: 1.

### I16F_P90S_V93I

In some embodiments, a KOD polymerase variant comprises: a F at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and an I at position V93 of SEQ ID NO: 1. In some embodiments, a KOD polymerase variant comprises: a F at position I16 of SEQ ID NO: 1, a S at position P90 of SEQ ID NO: 1, and an I at position V93 of SEQ ID NO: 1.

### I16F_P90S_V93I_R97A

In some embodiments, a KOD polymerase variant comprises: a F at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a S at position P90 of SEQ ID NO: 1, an I at position V93 of SEQ ID NO: 1, and an A at position R97 of SEQ ID NO: 1.

### I16F_P90S_V93I_R97F

In some embodiments, a KOD polymerase variant comprises: a F at a position corresponding to I16 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a S at position P90 of SEQ ID NO: 1, an I at position V93 of SEQ ID NO: 1, and a F at position R97 of SEQ ID NO: 1.

### I16F_P90S_V93I_R97H

In some embodiments, a KOD polymerase variant comprises: a F at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, a S at position P90 of SEQ ID NO: 1, an I at position V93 of SEQ ID NO: 1, and a H at position R97 of SEQ ID NO: 1.

### I16F_P90S_V93I_R97W

In some embodiments, a KOD polymerase variant comprises: a F at a position corresponding to I16 of SEQ ID NO: 1, a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, a S at position P90 of SEQ ID NO: 1, an I at position V93 of SEQ ID NO: 1, and a W at position R97 of SEQ ID NO: 1.

### I16F_P90S_V93Q

In some embodiments, a KOD polymerase variant comprises: a F at a position corresponding to I16 of SEQ ID NO: 1, a S at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and a Q at position V93 of SEQ ID NO: 1. In some embodiments, a KOD polymerase variant comprises: a F at position I16 of SEQ ID NO: 1, a S at position P90 of SEQ ID NO: 1, and a Q at position V93 of SEQ ID NO: 1.

### I16F_P90S_V93R

In some embodiments, a KOD polymerase variant comprises: a F at a position corresponding to I16 of SEQ ID NO: 1, a S at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, and a R at position V93 of SEQ ID NO: 1. In some embodiments, a KOD polymerase variant comprises: a F at position I16 of SEQ ID NO: 1, a S at position P90 of SEQ ID NO: 1, and a R at position V93 of SEQ ID NO: 1.

### I16F_P90S_V93R_R97F

In some embodiments, a KOD polymerase variant comprises: a F at a position corresponding to I16 of SEQ ID NO: 1, a S at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a S at position P90 of SEQ ID NO: 1, a R at position V93 of SEQ ID NO: 1, and a F at position R97 of SEQ ID NO: 1.

### I16F_P90S_V93R_R97H

In some embodiments, a KOD polymerase variant comprises: a F at a position corresponding to I16 of SEQ ID NO: 1, a S at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, a S at position P90 of SEQ ID NO: 1, a R at position V93 of SEQ ID NO: 1, and a H at position R97 of SEQ ID NO: 1.

### I16F_P90S_V93R_R97W

In some embodiments, a KOD polymerase variant comprises: a F at a position corresponding to I16 of SEQ ID NO: 1, a S at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a S at position P90 of SEQ ID NO: 1, a R at position V93 of SEQ ID NO: 1, and a W at position R97 of SEQ ID NO: 1. In some embodiments, a KOD polymerase variant comprises: a F at position I16 of SEQ ID NO: 1, a S at position P90 of SEQ ID NO: 1, a R at position V93 of SEQ ID NO: 1, and a W at position R97 of SEQ ID NO: 1.

### I16F_P90W

In some embodiments, a KOD polymerase variant comprises: a F at a position corresponding to I16 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a W at position P90 of SEQ ID NO: 1. In some embodiments, a KOD polymerase variant comprises: a F at position I16 of SEQ ID NO: 1, and a W at position P90 of SEQ ID NO: 1.

### I16F_P90W_R97A

In some embodiments, a KOD polymerase variant comprises: a F at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and an A at position R97 of SEQ ID NO: 1. In some embodiments, a KOD polymerase variant comprises: a F at position I16 of SEQ ID NO: 1, a W at position P90 of SEQ ID NO: 1, and an A at position R97 of SEQ ID NO: 1.

### I16F_P90W_R97F

In some embodiments, a KOD polymerase variant comprises: a F at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a F at position R97 of SEQ ID NO: 1. In some embodiments, a KOD polymerase variant comprises: a F at position I16 of SEQ ID NO: 1, a W at position P90 of SEQ ID NO: 1, and a F at position R97 of SEQ ID NO: 1.

### I16F_P90W_R97H

In some embodiments, a KOD polymerase variant comprises: a F at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a H at position R97 of SEQ ID NO: 1. In some embodiments, a KOD polymerase variant comprises: a F at position I16 of SEQ ID NO: 1, a W at position P90 of SEQ ID NO: 1, and a H at position R97 of SEQ ID NO: 1.

### I16F_P90W_R97W

In some embodiments, a KOD polymerase variant comprises: a F at a position corresponding to I16 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a W at position R97 of SEQ ID NO: 1. In some embodiments, a KOD polymerase variant comprises: a F at position I16 of SEQ ID NO: 1, a W at position P90 of SEQ ID NO: 1, and a W at position R97 of SEQ ID NO: 1.

### I16F_P90W_V93E_R97F

In some embodiments, a KOD polymerase variant comprises: a F at a position corresponding to I16 of SEQ ID NO: 1, a W at a position corresponding to P90 of SEQ ID NO: 1, an E at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, an E at position V93 of SEQ ID NO: 1, and a F at position R97 of SEQ ID NO: 1. In some embodiments, a KOD polymerase variant comprises: a F at position I16 of SEQ ID NO: 1, a W at position P90 of SEQ ID NO: 1, an E at position V93 of SEQ ID NO: 1, and a F at position R97 of SEQ ID NO: 1.

### I16F_P90W_V93I

In some embodiments, a KOD polymerase variant comprises: a F at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and an I at position V93 of SEQ ID NO: 1. In some embodiments, a KOD polymerase variant comprises: a F at position I16 of SEQ ID NO: 1, a W at position P90 of SEQ ID NO: 1, and an I at position V93 of SEQ ID NO: 1.

### I16F_P90W_V93I_R97A

In some embodiments, a KOD polymerase variant comprises: a F at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a W at position P90 of SEQ ID NO: 1, an I at position V93 of SEQ ID NO: 1, and an A at position R97 of SEQ ID NO: 1.

### I16F_P90W_V93I_R97F

In some embodiments, a KOD polymerase variant comprises: a F at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a W at position P90 of SEQ ID NO: 1, an I at position V93 of SEQ ID NO: 1, and a F at position R97 of SEQ ID NO: 1.

### I16F_P90W_V93I_R97H

In some embodiments, a KOD polymerase variant comprises: a F at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, a W at position P90 of SEQ ID NO: 1, an I at position V93 of SEQ ID NO: 1, and a H at position R97 of SEQ ID NO: 1.

### I16F_P90W_V93I_R97W

In some embodiments, a KOD polymerase variant comprises: a F at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, a W at position P90 of SEQ ID NO: 1, an I at position V93 of SEQ ID NO: 1, and a W at position R97 of SEQ ID NO: 1.

### I16F_P90W_V93K

In some embodiments, a KOD polymerase variant comprises: a F at a position corresponding to I16 of SEQ ID NO: 1, a W at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a K at position V93 of SEQ ID NO: 1. In some embodiments, a KOD polymerase variant comprises: a F at position I16 of SEQ ID NO: 1, a W at position P90 of SEQ ID NO: 1, and a K at position V93 of SEQ ID NO: 1.

### I16F_P90W_V93K_R97F

In some embodiments, a KOD polymerase variant comprises: a F at a position corresponding to I16 of SEQ ID NO: 1, a W at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, a W at position P90 of SEQ ID NO: 1, a K at position V93 of SEQ ID NO: 1, and a F at position R97 of SEQ ID NO: 1.

### I16F_P90W_V93K_R97W

In some embodiments, a KOD polymerase variant comprises: a F at a position corresponding to I16 of SEQ ID NO: 1, a W at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a K at position V93 of SEQ ID NO: 1, and a W at position R97 of SEQ ID NO: 1. In some embodiments, a KOD polymerase variant comprises: a F at position I16 of SEQ ID NO: 1, a W at position P90 of SEQ ID NO: 1, a K at position V93 of SEQ ID NO: 1, and a W at position R97 of SEQ ID NO: 1.

### I16F_P90W_V93Q

In some embodiments, a KOD polymerase variant comprises: a F at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a Q at position V93 of SEQ ID NO: 1. In some embodiments, a KOD polymerase variant comprises: a F at position I16 of SEQ ID NO: 1, a W at position P90 of SEQ ID NO: 1, and a Q at position V93 of SEQ ID NO: 1.

### I16F_P90W_V93Q_R97A

In some embodiments, a KOD polymerase variant comprises: a F at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, a W at position P90 of SEQ ID NO: 1, a Q at position V93 of SEQ ID NO: 1, and an A at position R97 of SEQ ID NO: 1.

### I16F_P90W_V93Q_R97F

In some embodiments, a KOD polymerase variant comprises: a F at a position corresponding to I16 of SEQ ID NO: 1, a W at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, a W at position P90 of SEQ ID NO: 1, a Q at position V93 of SEQ ID NO: 1, and a F at position R97 of SEQ ID NO: 1.

### I16F_P90W_V93Q_R97W

In some embodiments, a KOD polymerase variant comprises: a F at a position corresponding to I16 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: a F at position I16 of SEQ ID NO: 1, a W at position P90 of SEQ ID NO: 1, a Q at position V93 of SEQ ID NO: 1, and a W at position R97 of SEQ ID NO: 1.

### I16F_P90W_V93R

In some embodiments, a KOD polymerase variant comprises: a F at a position corresponding to I16 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, and a R at position V93 of SEQ ID NO: 1. In some embodiments, a KOD polymerase variant comprises: a F at position I16 of SEQ ID NO: 1, a W at position P90 of SEQ ID NO: 1, and a R at position V93 of SEQ ID NO: 1.

### I16F_P90W_V93R_R97A

In some embodiments, a KOD polymerase variant comprises: a F at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, a W at position P90 of SEQ ID NO: 1, a R at position V93 of SEQ ID NO: 1, and an A at position R97 of SEQ ID NO: 1.

### I16F_P90W_V93R_R97H

In some embodiments, a KOD polymerase variant comprises: a F at a position corresponding to I16 of SEQ ID NO: 1, a W at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, a W at position P90 of SEQ ID NO: 1, a R at position V93 of SEQ ID NO: 1, and a H at position R97 of SEQ ID NO: 1.

### I16F_P90W_V93R_R97W

In some embodiments, a KOD polymerase variant comprises: a F at a position corresponding to I16 of SEQ ID NO: 1, a W at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a W at position P90 of SEQ ID NO: 1, a R at position V93 of SEQ ID NO: 1, and a W at position R97 of SEQ ID NO: 1. In some embodiments, a KOD polymerase variant comprises: a F at position I16 of SEQ ID NO: 1, a W at position P90 of SEQ ID NO: 1, a R at position V93 of SEQ ID NO: 1, and a W at position R97 of SEQ ID NO: 1.

### I16F_P90Y

In some embodiments, a KOD polymerase variant comprises: a F at a position corresponding to I16 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a Y at position P90 of SEQ ID NO: 1. In some embodiments, a KOD polymerase variant comprises: a F at position I16 of SEQ ID NO: 1, and a Y at position P90 of SEQ ID NO: 1.

### I16F_P90Y_R97A

In some embodiments, a KOD polymerase variant comprises: a F at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and an A at position R97 of SEQ ID NO: 1. In some embodiments, a KOD polymerase variant comprises: a F at position I16 of SEQ ID NO: 1, a Y at position P90 of SEQ ID NO: 1, and an A at position R97 of SEQ ID NO: 1.

### I16F_P90Y_R97F

In some embodiments, a KOD polymerase variant comprises: a F at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and a F at position R97 of SEQ ID NO: 1. In some embodiments, a KOD polymerase variant comprises: a F at position I16 of SEQ ID NO: 1, a Y at position P90 of SEQ ID NO: 1, and a F at position R97 of SEQ ID NO: 1.

### I16F_P90Y_R97H

In some embodiments, a KOD polymerase variant comprises: a F at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and a H at position R97 of SEQ ID NO: 1. In some embodiments, a KOD polymerase variant comprises: a F at position I16 of SEQ ID NO: 1, a Y at position P90 of SEQ ID NO: 1, and a H at position R97 of SEQ ID NO: 1.

### I16F_P90Y_V93I

In some embodiments, a KOD polymerase variant comprises: a F at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and an I at position V93 of SEQ ID NO: 1. In some embodiments, a KOD polymerase variant comprises: a F at position I16 of SEQ ID NO: 1, a Y at position P90 of SEQ ID NO: 1, and an I at position V93 of SEQ ID NO: 1.

### I16F_P90Y_V93I_R97A

In some embodiments, a KOD polymerase variant comprises: a F at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, a Y at position P90 of SEQ ID NO: 1, an I at position V93 of SEQ ID NO: 1, and an A at position R97 of SEQ ID NO: 1.

### I16F_P90Y_V93I_R97F

In some embodiments, a KOD polymerase variant comprises: a F at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, a Y at position P90 of SEQ ID NO: 1, an I at position V93 of SEQ ID NO: 1, and a F at position R97 of SEQ ID NO: 1.

### I16F_P90Y_V93I_R97H

In some embodiments, a KOD polymerase variant comprises: a F at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, a Y at position P90 of SEQ ID NO: 1, an I at position V93 of SEQ ID NO: 1, and a H at position R97 of SEQ ID NO: 1.

### I16F_P90Y_V93I_R97W

In some embodiments, a KOD polymerase variant comprises: a F at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, a Y at position P90 of SEQ ID NO: 1, an I at position V93 of SEQ ID NO: 1, and a W at position R97 of SEQ ID NO:1.

### I16F_P90Y_V93K_R97A

In some embodiments, a KOD polymerase variant comprises: a F at a position corresponding to I16 of SEQ ID NO: 1, a Y at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a K at position V93 of SEQ ID NO: 1, and an A at position R97 of SEQ ID NO: 1. In some embodiments, a KOD polymerase variant comprises: a F at position I16 of SEQ ID NO: 1, a Y at position P90 of SEQ ID NO: 1, a K at position V93 of SEQ ID NO: 1, and an A at position R97 of SEQ ID NO:1.

### I16F_P90Y_V93K_R97F

In some embodiments, a KOD polymerase variant comprises: a F at a position corresponding to I16 of SEQ ID NO: 1, a Y at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, a Y at position P90 of SEQ ID NO: 1, a K at position V93 of SEQ ID NO: 1, and a F at position R97 of SEQ ID NO: 1.

### I16F_P90Y_V93Q

In some embodiments, a KOD polymerase variant comprises: a F at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and a Q at position V93 of SEQ ID NO: 1. In some embodiments, a KOD polymerase variant comprises: a F at position I16 of SEQ ID NO: 1, a Y at position P90 of SEQ ID NO: 1, and a Q at position V93 of SEQ ID NO: 1.

### I16F_P90Y_V93Q_R97A

In some embodiments, a KOD polymerase variant comprises: a F at a position corresponding to I16 of SEQ ID NO: 1, a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, a Y at position P90 of SEQ ID NO: 1, a Q at position V93 of SEQ ID NO: 1, and an A at position R97 of SEQ ID NO:1.

### I16F_P90Y_V93Q_R97F

In some embodiments, a KOD polymerase variant comprises: a F at a position corresponding to I16 of SEQ ID NO: 1, a Y at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, a Y at position P90 of SEQ ID NO: 1, a Q at position V93 of SEQ ID NO: 1, and a F at position R97 of SEQ ID NO: 1.

### I16F_P90Y_V93Q_R97H

In some embodiments, a KOD polymerase variant comprises: a F at a position corresponding to I16 of SEQ ID NO: 1, a Y at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a Q at position V93 of SEQ ID NO: 1, and a H at position R97 of SEQ ID NO: 1. In some embodiments, a KOD polymerase variant comprises: a F at position I16 of SEQ ID NO: 1, a Y at position P90 of SEQ ID NO: 1, a Q at position V93 of SEQ ID NO: 1, and a H at position R97 of SEQ ID NO: 1.

### I16F_P90Y_V93Q_R97W

In some embodiments, a KOD polymerase variant comprises: a F at a position corresponding to I16 of SEQ ID NO: 1, a Y at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a Q at position V93 of SEQ ID NO: 1, and a W at position R97 of SEQ ID NO: 1. In some embodiments, a KOD polymerase variant comprises: a F at position I16 of SEQ ID NO: 1, a Y at position P90 of SEQ ID NO: 1, a Q at position V93 of SEQ ID NO: 1, and a W at position R97 of SEQ ID NO: 1.

### I16F_P90Y_V93R

In some embodiments, a KOD polymerase variant comprises: a F at a position corresponding to I16 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, and a R at position V93 of SEQ ID NO: 1. In some embodiments, a KOD polymerase variant comprises: a F at position I16 of SEQ ID NO: 1, a Y at position P90 of SEQ ID NO: 1, and a R at position V93 of SEQ ID NO: 1.

### I16F_P90Y_V93R_R97A

In some embodiments, a KOD polymerase variant comprises: a F at a position corresponding to I16 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, a Y at position P90 of SEQ ID NO: 1, a R at position V93 of SEQ ID NO: 1, and an A at position R97 of SEQ ID NO: 1.

### I16F_P90Y_V93R_R97F

In some embodiments, a KOD polymerase variant comprises: a F at a position corresponding to I16 of SEQ ID NO: 1, a Y at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Y at position P90 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a Y at position P90 of SEQ ID NO: 1, a R at position V93 of SEQ ID NO: 1, and a F at position R97 of SEQ ID NO: 1.

### I16F_R97A

In some embodiments, a KOD polymerase variant comprises: a F at a position corresponding to I16 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and an A at position R97 of SEQ ID NO: 1. In some embodiments, a KOD polymerase variant comprises: a F at position I16 of SEQ ID NO: 1, and an A at position R97 of SEQ ID NO: 1.

### I16F_R97A_R119M

In some embodiments, a KOD polymerase variant comprises: a F at a position corresponding to I16 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an A at position R97 of SEQ ID NO: 1, and a M at position R119 of SEQ ID NO: 1. In some embodiments, a KOD polymerase variant comprises: a F at position I16 of SEQ ID NO: 1, an A at position R97 of SEQ ID NO: 1, and a M at position R119 of SEQ ID NO: 1.

### I16F_R97F

In some embodiments, a KOD polymerase variant comprises: a F at a position corresponding to I16 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a F at position R97 of SEQ ID NO: 1. In some embodiments, a KOD polymerase variant comprises: a F at position I16 of SEQ ID NO: 1, and a F at position R97 of SEQ ID NO: 1.

### I16F_R97F_R119Q

In some embodiments, a KOD polymerase variant comprises: a F at a position corresponding to I16 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a F at position R97 of SEQ ID NO: 1, and a Q at position R119 of SEQ ID NO: 1. In some embodiments, a KOD polymerase variant comprises: a F at position I16 of SEQ ID NO: 1, a F at position R97 of SEQ ID NO: 1, and a Q at position R119 of SEQ ID NO: 1.

### I16F_R97H

In some embodiments, a KOD polymerase variant comprises: a F at a position corresponding to I16 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a H at position R97 of SEQ ID NO: 1. In some embodiments, a KOD polymerase variant comprises: a F at position I16 of SEQ ID NO: 1, and a H at position R97 of SEQ ID NO: 1.

### I16F_R97W

In some embodiments, a KOD polymerase variant comprises: a F at a position corresponding to I16 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a W at position R97 of SEQ ID NO: 1. In some embodiments, a KOD polymerase variant comprises: a F at position I16 of SEQ ID NO: 1, and a W at position R97 of SEQ ID NO: 1.

### I16F_R119Q

In some embodiments, a KOD polymerase variant comprises: a F at a position corresponding to I16 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a Q at position R119 of SEQ ID NO: 1. In some embodiments, a KOD polymerase variant comprises: a F at position I16 of SEQ ID NO: 1, and a Q at position R119 of SEQ ID NO: 1.

### I16F_V93E

In some embodiments, a KOD polymerase variant comprises: a F at a position corresponding to I16 of SEQ ID NO: 1 and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and an E at position V93 of SEQ ID NO: 1. In some embodiments, a KOD polymerase variant comprises: a F at position I16 of SEQ ID NO: 1, and an E at position V93 of SEQ ID NO: 1.

### I16F_V93E_R97A

In some embodiments, a KOD polymerase variant comprises: a F at a position corresponding to I16 of SEQ ID NO: 1, an E at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an E at position V93 of SEQ ID NO: 1, and an A at position R97 of SEQ ID NO: 1. In some embodiments, a KOD polymerase variant comprises: a F at position I16 of SEQ ID NO: 1, an E at position V93 of SEQ ID NO: 1, and an A at position R97 of SEQ ID NO: 1.

### I16F_V93E_R97F

In some embodiments, a KOD polymerase variant comprises: a F at a position corresponding to I16 of SEQ ID NO: 1, an E at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an E at position V93 of SEQ ID NO: 1, and a F at position R97 of SEQ ID NO: 1. In some embodiments, a KOD polymerase variant comprises: a F at position I16 of SEQ ID NO: 1, an E at position V93 of SEQ ID NO: 1, and a F at position R97 of SEQ ID NO: 1.

### I16F_V93E_R97H

In some embodiments, a KOD polymerase variant comprises: a F at a position corresponding to I16 of SEQ ID NO: 1, an E at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an E at position V93 of SEQ ID NO: 1, and a H at position R97 of SEQ ID NO: 1. In some embodiments, a KOD polymerase variant comprises: a F at position I16 of SEQ ID NO: 1, an E at position V93 of SEQ ID NO: 1, and a H at position R97 of SEQ ID NO: 1.

### I16F_V93I

In some embodiments, a KOD polymerase variant comprises: a F at a position corresponding to I16 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and an I at position V93 of SEQ ID NO: 1. In some embodiments, a KOD polymerase variant comprises: a F at position I16 of SEQ ID NO: 1, and an I at position V93 of SEQ ID NO: 1.

### I16F_V93I_R97A

In some embodiments, a KOD polymerase variant comprises: a F at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and an A at position R97 of SEQ ID NO: 1. In some embodiments, a KOD polymerase variant comprises: a F at position I16 of SEQ ID NO: 1, an I at position V93 of SEQ ID NO: 1, and an A at position R97 of SEQ ID NO: 1.

### I16F_V93I_R97F

In some embodiments, a KOD polymerase variant comprises: a F at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and a F at position R97 of SEQ ID NO: 1. In some embodiments, a KOD polymerase variant comprises: a F at position I16 of SEQ ID NO: 1, an I at position V93 of SEQ ID NO: 1, and a F at position R97 of SEQ ID NO: 1.

### I16F_V93I_R97F_R119Q

In some embodiments, a KOD polymerase variant comprises: a F at a position corresponding to I16 of SEQ ID NO: 1, an I at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, a F at position R97 of SEQ ID NO: 1, and a Q at position R119 of SEQ ID NO: 1. In some embodiments, a KOD polymerase variant comprises: a F at position I16 of SEQ ID NO: 1, an I at position V93 of SEQ ID NO: 1, a F at position R97 of SEQ ID NO: 1, and a Q at position R119 of SEQ ID NO: 1.

### I16F_V93I_R97H

In some embodiments, a KOD polymerase variant comprises: a F at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and a H at position R97 of SEQ ID NO: 1. In some embodiments, a KOD polymerase variant comprises: a F at position I16 of SEQ ID NO: 1, an I at position V93 of SEQ ID NO: 1, and a H at position R97 of SEQ ID NO: 1.

### I16F_V93I_R97P

In some embodiments, a KOD polymerase variant comprises: a F at a position corresponding to I16 of SEQ ID NO: 1, an I at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and a P at position R97 of SEQ ID NO: 1. In some embodiments, a KOD polymerase variant comprises: a F at position I16 of SEQ ID NO: 1, an I at position V93 of SEQ ID NO: 1, and a P at position R97 of SEQ ID NO: 1.

### I16F_V93I_R97W

In some embodiments, a KOD polymerase variant comprises: a F at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and a W at position R97 of SEQ ID NO: 1. In some embodiments, a KOD polymerase variant comprises: a F at position I16 of SEQ ID NO: 1, an I at position V93 of SEQ ID NO: 1, and a W at position R97 of SEQ ID NO: 1.

### I16F_V93I_R119Q

In some embodiments, a KOD polymerase variant comprises: a F at a position corresponding to I16 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, an I at position V93 of SEQ ID NO: 1, and a Q at position R119 of SEQ ID NO: 1. In some embodiments, a KOD polymerase variant comprises: a F at position I16 of SEQ ID NO: 1, an I at position V93 of SEQ ID NO: 1, and a Q at position R119 of SEQ ID NO: 1.

### I16F_V93K

In some embodiments, a KOD polymerase variant comprises: a F at a position corresponding to I16 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a K at position V93 of SEQ ID NO: 1. In some embodiments, a KOD polymerase variant comprises: a F at position I16 of SEQ ID NO: 1, and a K at position V93 of SEQ ID NO: 1.

### I16F_V93K_R97A

In some embodiments, a KOD polymerase variant comprises: a F at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a K at position V93 of SEQ ID NO: 1, and an A at position R97 of SEQ ID NO: 1. In some embodiments, a KOD polymerase variant comprises: a F at position I16 of SEQ ID NO: 1, a K at position V93 of SEQ ID NO: 1, and an A at position R97 of SEQ ID NO: 1.

### I16F_V93K_R97F

In some embodiments, a KOD polymerase variant comprises: a F at a position corresponding to I16 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a K at position V93 of SEQ ID NO: 1, and a F at position R97 of SEQ ID NO: 1. In some embodiments, a KOD polymerase variant comprises: a F at position I16 of SEQ ID NO: 1, a K at position V93 of SEQ ID NO: 1, and a F at position R97 of SEQ ID NO: 1.

### I16F_V93K_R97H

In some embodiments, a KOD polymerase variant comprises: a F at a position corresponding to I16 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a K at position V93 of SEQ ID NO: 1, and a H at position R97 of SEQ ID NO: 1. In some embodiments, a KOD polymerase variant comprises: a F at position I16 of SEQ ID NO: 1, a K at position V93 of SEQ ID NO: 1, and a H at position R97 of SEQ ID NO: 1.

### I16F_V93K_R97W

In some embodiments, a KOD polymerase variant comprises: a F at a position corresponding to I16 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a K at position V93 of SEQ ID NO: 1, and a W at position R97 of SEQ ID NO: 1. In some embodiments, a KOD polymerase variant comprises: a F at position I16 of SEQ ID NO: 1, a K at position V93 of SEQ ID NO: 1, and a W at position R97 of SEQ ID NO: 1.

### I16F_V93Q

In some embodiments, a KOD polymerase variant comprises: a F at a position corresponding to I16 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a Q at position V93 of SEQ ID NO: 1. In some embodiments, a KOD polymerase variant comprises: a F at position I16 of SEQ ID NO: 1, and a Q at position V93 of SEQ ID NO: 1.

### I16F_V93Q_R97A

In some embodiments, a KOD polymerase variant comprises: a F at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Q at position V93 of SEQ ID NO: 1, and an A at position R97 of SEQ ID NO: 1. In some embodiments, a KOD polymerase variant comprises: a F at position I16 of SEQ ID NO: 1, a Q at position V93 of SEQ ID NO: 1, and an A at position R97 of SEQ ID NO: 1.

### I16F_V93Q_R97F

In some embodiments, a KOD polymerase variant comprises: a F at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Q at position V93 of SEQ ID NO: 1, and a F at position R97 of SEQ ID NO: 1. In some embodiments, a KOD polymerase variant comprises: a F at position I16 of SEQ ID NO: 1, a Q at position V93 of SEQ ID NO: 1, and a F at position R97 of SEQ ID NO: 1.

### I16F_V93Q_R97H

In some embodiments, a KOD polymerase variant comprises: a F at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Q at position V93 of SEQ ID NO: 1, and a H at position R97 of SEQ ID NO: 1. In some embodiments, a KOD polymerase variant comprises: a F at position I16 of SEQ ID NO: 1, a Q at position V93 of SEQ ID NO: 1, and a H at position R97 of SEQ ID NO: 1.

### I16F_V93Q_R97W

In some embodiments, a KOD polymerase variant comprises: a F at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a Q at position V93 of SEQ ID NO: 1, and a W at position R97 of SEQ ID NO: 1. In some embodiments, a KOD polymerase variant comprises: a F at position I16 of SEQ ID NO: 1, a Q at position V93 of SEQ ID NO: 1, and a W at position R97 of SEQ ID NO: 1.

### I16F_V93R

In some embodiments, a KOD polymerase variant comprises: a F at a position corresponding to I16 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, and a R at position V93 of SEQ ID NO: 1. In some embodiments, a KOD polymerase variant comprises: a F at position I16 of SEQ ID NO: 1, and a R at position V93 of SEQ ID NO: 1.

### I16F_V93R_R97A

In some embodiments, a KOD polymerase variant comprises: a F at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, and an A at position R97 of SEQ ID NO: 1. In some embodiments, a KOD polymerase variant comprises: a F at position I16 of SEQ ID NO: 1, a R at position V93 of SEQ ID NO: 1, and an A at position R97 of SEQ ID NO: 1.

### I16F_V93R_R97A_R119M

In some embodiments, a KOD polymerase variant comprises: a F at a position corresponding to I16 of SEQ ID NO: 1, a R at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, an A at position R97 of SEQ ID NO: 1, and a M at position R119 of SEQ ID NO: 1. In some embodiments, a KOD polymerase variant comprises: a F at position I16 of SEQ ID NO: 1, a R at position V93 of SEQ ID NO: 1, an A at position R97 of SEQ ID NO: 1, and a M at position R119 of SEQ ID NO: 1.

### I16F_V93R_R97A_R119Q

In some embodiments, a KOD polymerase variant comprises: a F at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a F at position I16 of SEQ ID NO: 1, a R at position V93 of SEQ ID NO: 1, an A at position R97 of SEQ ID NO: 1, and a Q at position R119 of SEQ ID NO: 1.

### I16F_V93R_R97A_R119Y

In some embodiments, a KOD polymerase variant comprises: a F at a position corresponding to I16 of SEQ ID NO: 1, a R at a position corresponding to V93 of SEQ ID NO: 1, an A at a position corresponding to R97 of SEQ ID NO: 1, and a Y at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, an A at position R97 of SEQ ID NO: 1, and a Y at position R119 of SEQ ID NO: 1. In some embodiments, a KOD polymerase variant comprises: a F at position I16 of SEQ ID NO: 1, a R at position V93 of SEQ ID NO: 1, an A at position R97 of SEQ ID NO: 1, and a Y at position R119 of SEQ ID NO: 1.

### I16F_V93R_R97F

In some embodiments, a KOD polymerase variant comprises: a F at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, and a F at position R97 of SEQ ID NO: 1. In some embodiments, a KOD polymerase variant comprises: a F at position I16 of SEQ ID NO: 1, a R at position V93 of SEQ ID NO: 1, and a F at position R97 of SEQ ID NO: 1.

### I16F_V93R_R97F_R119Q

In some embodiments, a KOD polymerase variant comprises: a F at a position corresponding to I16 of SEQ ID NO: 1, a R at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, a F at position R97 of SEQ ID NO: 1, and a Q at position R119 of SEQ ID NO: 1. In some embodiments, a KOD polymerase variant comprises: a F at position I16 of SEQ ID NO: 1, a R at position V93 of SEQ ID NO: 1, a F at position R97 of SEQ ID NO: 1, and a Q at position R119 of SEQ ID NO: 1.

### I16F_V93R_R97H

In some embodiments, a KOD polymerase variant comprises: a F at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, and a H at position R97 of SEQ ID NO: 1. In some embodiments, a KOD polymerase variant comprises: a F at position I16 of SEQ ID NO: 1, a R at position V93 of SEQ ID NO: 1, and a H at position R97 of SEQ ID NO: 1.

### I16F_V93R_R119M

In some embodiments, a KOD polymerase variant comprises: a F at a position corresponding to I16 of SEQ ID NO: 1, a R at a position corresponding to V93 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, and a M at position R119 of SEQ ID NO: 1. In some embodiments, a KOD polymerase variant comprises: a F at position I16 of SEQ ID NO: 1, a R at position V93 of SEQ ID NO: 1, and a M at position R119 of SEQ ID NO: 1.

### I16F_V93R_R119Q

In some embodiments, a KOD polymerase variant comprises: a F at a position corresponding to I16 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position I16 of SEQ ID NO: 1, a R at position V93 of SEQ ID NO: 1, and a Q at position R119 of SEQ ID NO: 1. In some embodiments, a KOD polymerase variant comprises: a F at position I16 of SEQ ID NO: 1, a R at position V93 of SEQ ID NO: 1, and a Q at position R119 of SEQ ID NO: 1. I16H_P90F_V93I

In some embodiments, a KOD polymerase variant comprises: a H at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, a F at position P90 of SEQ ID NO: 1, and an I at position V93 of SEQ ID NO: 1. In some embodiments, a KOD polymerase variant comprises: a H at position I16 of SEQ ID NO: 1, a F at position P90 of SEQ ID NO: 1, and an I at position V93 of SEQ ID NO: 1. I16H_P90W_V93I

In some embodiments, a KOD polymerase variant comprises: a H at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, a W at position P90 of SEQ ID NO: 1, and an I at position V93 of SEQ ID NO: 1. In some embodiments, a KOD polymerase variant comprises: a H at position I16 of SEQ ID NO: 1, a W at position P90 of SEQ ID NO: 1, and an I at position V93 of SEQ ID NO: 1. I16H_R97A

In some embodiments, a KOD polymerase variant comprises: a H at a position corresponding to I16 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, and an A at position R97 of SEQ ID NO: 1. In some embodiments, a KOD polymerase variant comprises: a H at position I16 of SEQ ID NO: 1, and an A at position R97 of SEQ ID NO: 1. I16H_R97H

In some embodiments, a KOD polymerase variant comprises: a H at a position corresponding to I16 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, and a H at position R97 of SEQ ID NO: 1. In some embodiments, a KOD polymerase variant comprises: a H at position I16 of SEQ ID NO: 1, and a H at position R97 of SEQ ID NO: 1.

### I16H_V93I_R97A

In some embodiments, a KOD polymerase variant comprises: a H at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, an I at position V93 of SEQ ID NO: 1, and an A at position R97 of SEQ ID NO: 1. In some embodiments, a KOD polymerase variant comprises: a H at position I16 of SEQ ID NO: 1, an I at position V93 of SEQ ID NO: 1, and an A at position R97 of SEQ ID NO: 1.

### I16H_V93I_R97H

In some embodiments, a KOD polymerase variant comprises: a H at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, an I at position V93 of SEQ ID NO: 1, and a H at position R97 of SEQ ID NO: 1. In some embodiments, a KOD polymerase variant comprises: a H at position I16 of SEQ ID NO: 1, an I at position V93 of SEQ ID NO: 1, and a H at position R97 of SEQ ID NO: 1.

### I16H_V93Q

In some embodiments, a KOD polymerase variant comprises: a H at a position corresponding to I16 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position I16 of SEQ ID NO: 1, and a Q at position V93 of SEQ ID NO: 1. In some embodiments, a KOD polymerase variant comprises: a H at position I16 of SEQ ID NO: 1, and a Q at position V93 of SEQ ID NO: 1.

### I16L

In some embodiments, a KOD polymerase variant comprises: a L at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1. In some embodiments, a KOD polymerase variant comprises: a L at position I16 of SEQ ID NO: 1.

### I16L_P36A_P90F

In some embodiments, a KOD polymerase variant comprises: a L at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a F at position P90 of SEQ ID NO: 1. In some embodiments, a KOD polymerase variant comprises: a L at position I16 of SEQ ID NO: 1, an A at position P36 of SEQ ID NO: 1, and a F at position P90 of SEQ ID NO: 1.

### I16L_P36A_P90F_V93I

In some embodiments, a KOD polymerase variant comprises: a L at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a L at position I16 of SEQ ID NO: 1, an A at position P36 of SEQ ID NO: 1, a F at position P90 of SEQ ID NO: 1, and an I at position V93 of SEQ ID NO: 1.

### I16L_P36A_P90F_V93I_R97A

In some embodiments, a KOD polymerase variant comprises: a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1.

### I16L_P36A_P90F_V93I_R97F

In some embodiments, a KOD polymerase variant comprises: a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1.

### I16L_P36A_P90F_V93I_R97H

In some embodiments, a KOD polymerase variant comprises: a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### I16L_P36A_P90I_R97A

In some embodiments, a KOD polymerase variant comprises: a L at a position corresponding to I16 of SEQ ID NO: 1, an A at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position P90 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, an I at position P90 of SEQ ID NO: 1, and an A at position R97 of SEQ ID NO: 1.

### I16L_P36A_P90I_V93I

In some embodiments, a KOD polymerase variant comprises: a L at a position corresponding to I16 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a L at position I16 of SEQ ID NO: 1, an A at position P36 of SEQ ID NO: 1, an I at position P90 of SEQ ID NO: 1, and an I at position V93 of SEQ ID NO: 1.

### I16L_P36A_P90I_V93I_R97A

In some embodiments, a KOD polymerase variant comprises: a L at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1.

### I16L_P36A_P90W

In some embodiments, a KOD polymerase variant comprises: a L at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a W at position P90 of SEQ ID NO: 1. In some embodiments, a KOD polymerase variant comprises: a L at position I16 of SEQ ID NO: 1, an A at position P36 of SEQ ID NO: 1, and a W at position P90 of SEQ ID NO: 1.

### I16L_P36A_P90W_V93I

In some embodiments, a KOD polymerase variant comprises: a L at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a L at position I16 of SEQ ID NO: 1, an A at position P36 of SEQ ID NO: 1, a W at position P90 of SEQ ID NO: 1, and an I at position V93 of SEQ ID NO: 1.

### I16L_P36A_P90Y_V93I

In some embodiments, a KOD polymerase variant comprises: a L at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a L at position I16 of SEQ ID NO: 1, an A at position P36 of SEQ ID NO: 1, a Y at position P90 of SEQ ID NO: 1, and an I at position V93 of SEQ ID NO: 1.

### I16L_P36A_R97A

In some embodiments, a KOD polymerase variant comprises: a L at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, and an A at position R97 of SEQ ID NO: 1.

### I16L_P36A_R97F

In some embodiments, a KOD polymerase variant comprises: a L at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, and a F at position R97 of SEQ ID NO: 1.

### I16L_P36A_R97W

In some embodiments, a KOD polymerase variant comprises: a L at a position corresponding to I16 of SEQ ID NO: 1, an A at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a W at position R97 of SEQ ID NO: 1. In some embodiments, a KOD polymerase variant comprises: a L at position I16 of SEQ ID NO: 1, an A at position P36 of SEQ ID NO: 1, and a W at position R97 of SEQ ID NO: 1.

### I16L_P36A_V93E

In some embodiments, a KOD polymerase variant comprises: a L at a position corresponding to I16 of SEQ ID NO: 1, an A at a position corresponding to P36 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and an E at position V93 of SEQ ID NO: 1. In some embodiments, a KOD polymerase variant comprises: a L at position I16 of SEQ ID NO: 1, an A at position P36 of SEQ ID NO: 1, and an E at position V93 of SEQ ID NO: 1.

### I16L_P36A_V93I_R97A

In some embodiments, a KOD polymerase variant comprises: a L at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, an I at position V93 of SEQ ID NO: 1, and an A at position R97 of SEQ ID NO: 1.

### I16L_P36A_V93I_R97F

In some embodiments, a KOD polymerase variant comprises: a L at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, an I at position V93 of SEQ ID NO: 1, and a F at position R97 of SEQ ID NO: 1.

### I16L_P36A_V93I_R97W

In some embodiments, a KOD polymerase variant comprises: a L at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, an A at position P36 of SEQ ID NO: 1, an I at position V93 of SEQ ID NO: 1, and a W at position R97 of SEQ ID NO: 1.

### I16L_P36A_V93K

In some embodiments, a KOD polymerase variant comprises: a L at a position corresponding to I16 of SEQ ID NO: 1, an A at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a K at position V93 of SEQ ID NO: 1. In some embodiments, a KOD polymerase variant comprises: a L at position I16 of SEQ ID NO: 1, an A at position P36 of SEQ ID NO: 1, and a K at position V93 of SEQ ID NO: 1.

### I16L_P36A_V93K_R97F

In some embodiments, a KOD polymerase variant comprises: a L at a position corresponding to I16 of SEQ ID NO: 1, an A at a position corresponding to P36 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a K at position V93 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, a K at position V93 of SEQ ID NO: 1, and a F at position R97 of SEQ ID NO: 1.

### I16L_P36A_V93Q

In some embodiments, a KOD polymerase variant comprises: a L at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a Q at position V93 of SEQ ID NO: 1. In some embodiments, a KOD polymerase variant comprises: a L at position I16 of SEQ ID NO: 1, an A at position P36 of SEQ ID NO: 1, and a Q at position V93 of SEQ ID NO: 1.

### I16L_P36A_V93Q_R97A

In some embodiments, a KOD polymerase variant comprises: a L at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, a Q at position V93 of SEQ ID NO: 1, and an A at position R97 of SEQ ID NO: 1.

### I16L_P36A_V93Q_R97F

In some embodiments, a KOD polymerase variant comprises: a L at a position corresponding to I16 of SEQ ID NO: 1, an A at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, a Q at position V93 of SEQ ID NO: 1, and a F at position R97 of SEQ ID NO: 1.

### I16L_P36A_V93Q_R97H

In some embodiments, a KOD polymerase variant comprises: a L at a position corresponding to I16 of SEQ ID NO: 1, an A at a position corresponding to P36 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a Q at position V93 of SEQ ID NO: 1, and a H at position R97 of SEQ ID NO: 1. In some embodiments, a KOD polymerase variant comprises: a L at position I16 of SEQ ID NO: 1, an A at position P36 of SEQ ID NO: 1, a Q at position V93 of SEQ ID NO: 1, and a H at position R97 of SEQ ID NO: 1.

### I16L_P36A_V93R

In some embodiments, a KOD polymerase variant comprises: a L at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, and a R at position V93 of SEQ ID NO: 1. In some embodiments, a KOD polymerase variant comprises: a L at position I16 of SEQ ID NO: 1, an A at position P36 of SEQ ID NO: 1, and a R at position V93 of SEQ ID NO: 1.

### I16L_P36A_V93R_R97A

In some embodiments, a KOD polymerase variant comprises: a L at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, an A at position P36 of SEQ ID NO: 1, a R at position V93 of SEQ ID NO: 1, and an A at position R97 of SEQ ID NO: 1.

### I16L_P36A_V93R_R97A_R119Q

In some embodiments, a KOD polymerase variant comprises: a L at a position corresponding to I16 of SEQ ID NO: 1, an A at a position corresponding to P36 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a L at position I16 of SEQ ID NO: 1, an A at position P36 of SEQ ID NO: 1, a R at position V93 of SEQ ID NO: 1, an A at position R97 of SEQ ID NO: 1, and a Q at position R119 of SEQ ID NO: 1.

### I16L_P36A_V93R_R97F

In some embodiments, a KOD polymerase variant comprises: a L at a position corresponding to I16 of SEQ ID NO: 1, an A at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a R at position V93 of SEQ ID NO: 1, and a F at position R97 of SEQ ID NO: 1. In some embodiments, a KOD polymerase variant comprises: a L at position I16 of SEQ ID NO: 1, an A at position P36 of SEQ ID NO: 1, a R at position V93 of SEQ ID NO: 1, and a F at position R97 of SEQ ID NO: 1.

### I16L_P36A_V93R_R97H

In some embodiments, a KOD polymerase variant comprises: a L at a position corresponding to I16 of SEQ ID NO: 1, an A at a position corresponding to P36 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an A at position P36 of SEQ ID NO: 1, a R at position V93 of SEQ ID NO: 1, and a H at position R97 of SEQ ID NO: 1. In some embodiments, a KOD polymerase variant comprises: a L at position I16 of SEQ ID NO: 1, an A at position P36 of SEQ ID NO: 1, a R at position V93 of SEQ ID NO: 1, and a H at position R97 of SEQ ID NO: 1.

### I16L_P36H

In some embodiments, a KOD polymerase variant comprises: a L at a position corresponding to I16 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a H at position P36 of SEQ ID NO: 1. In some embodiments, a KOD polymerase variant comprises: a L at position I16 of SEQ ID NO: 1, and a H at position P36 of SEQ ID NO: 1.

### I16L_P36H_P90F

In some embodiments, a KOD polymerase variant comprises: a L at a position corresponding to I16 of SEQ ID NO: 1, a H at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a F at position P90 of SEQ ID NO: 1. In some embodiments, a KOD polymerase variant comprises: a L at position I16 of SEQ ID NO: 1, a H at position P36 of SEQ ID NO: 1, and a F at position P90 of SEQ ID NO: 1.

### I16L_P36H_P90I_V93I_R97F

In some embodiments, a KOD polymerase variant comprises: a L at a position corresponding to I16 of SEQ ID NO: 1, a H at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a H at position P36 of SEQ ID NO: 1, an I at position P90 of SEQ ID NO: 1, an I at position V93 of SEQ ID NO: 1, and a F at position R97 of SEQ ID NO: 1.

### I16L_P36H_P90W

In some embodiments, a KOD polymerase variant comprises: a L at a position corresponding to I16 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a W at position P90 of SEQ ID NO: 1. In some embodiments, a KOD polymerase variant comprises: a L at position I16 of SEQ ID NO: 1, a H at position P36 of SEQ ID NO: 1, and a W at position P90 of SEQ ID NO: 1.

### I16L_P36H_P90W_V93I

In some embodiments, a KOD polymerase variant comprises: a L at a position corresponding to I16 of SEQ ID NO: 1, a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, a W at position P90 of SEQ ID NO: 1, and an I at position V93 of SEQ ID NO: 1. In some embodiments, a KOD polymerase variant comprises: a L at position I16 of SEQ ID NO: 1, a H at position P36 of SEQ ID NO: 1, a W at position P90 of SEQ ID NO: 1, and an I at position V93 of SEQ ID NO: 1.

### I16L_P36H_P90Y_V93I_R97A

In some embodiments, a KOD polymerase variant comprises: a L at a position corresponding to I16 of SEQ ID NO: 1, a H at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a H at position P36 of SEQ ID NO: 1, a Y at position P90 of SEQ ID NO: 1, an I at position V93 of SEQ ID NO: 1, and an A at position R97 of SEQ ID NO: 1.

### I16L_P36H_R97F

In some embodiments, a KOD polymerase variant comprises: a L at a position corresponding to I16 of SEQ ID NO: 1, a H at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a F at position R97 of SEQ ID NO: 1. In some embodiments, a KOD polymerase variant comprises: a L at position I16 of SEQ ID NO: 1, a H at position P36 of SEQ ID NO: 1, and a F at position R97 of SEQ ID NO: 1.

### I16L_P36H_R97H

In some embodiments, a KOD polymerase variant comprises: a L at a position corresponding to I16 of SEQ ID NO: 1, a H at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a H at position R97 of SEQ ID NO: 1. In some embodiments, a KOD polymerase variant comprises: a L at position I16 of SEQ ID NO: 1, a H at position P36 of SEQ ID NO: 1, and a H at position R97 of SEQ ID NO: 1.

### I16L_P36H_V93I

In some embodiments, a KOD polymerase variant comprises: a L at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and an I at position V93 of SEQ ID NO: 1. In some embodiments, a KOD polymerase variant comprises: a L at position I16 of SEQ ID NO: 1, a H at position P36 of SEQ ID NO: 1, and an I at position V93 of SEQ ID NO: 1.

### I16L_P36H_V93I_R97A

In some embodiments, a KOD polymerase variant comprises: a L at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a H at position P36 of SEQ ID NO: 1, an I at position V93 of SEQ ID NO: 1, and an A at position R97 of SEQ ID NO: 1.

### I16L_P36H_V93I_R97F

In some embodiments, a KOD polymerase variant comprises: a L at a position corresponding to I16 of SEQ ID NO: 1, a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a H at position P36 of SEQ ID NO: 1, an I at position V93 of SEQ ID NO: 1, and a F at position R97 of SEQ ID NO: 1.

### I16L_P36H_V93I_R97H

In some embodiments, a KOD polymerase variant comprises: a L at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a H at position P36 of SEQ ID NO: 1, an I at position V93 of SEQ ID NO: 1, and a H at position R97 of SEQ ID NO: 1.

### I16L_P36H_V93I_R97W

In some embodiments, a KOD polymerase variant comprises: a L at a position corresponding to I16 of SEQ ID NO: 1, a H at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a H at position P36 of SEQ ID NO: 1, an I at position V93 of SEQ ID NO: 1, and a W at position R97 of SEQ ID NO: 1.

### I16L_P36H_V93Q

In some embodiments, a KOD polymerase variant comprises: a L at a position corresponding to I16 of SEQ ID NO: 1, a H at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a Q at position V93 of SEQ ID NO: 1. In some embodiments, a KOD polymerase variant comprises: a L at position I16 of SEQ ID NO: 1, a H at position P36 of SEQ ID NO: 1, and a Q at position V93 of SEQ ID NO: 1.

### I16L_P36H_V93Q_R97F

In some embodiments, a KOD polymerase variant comprises: a L at a position corresponding to I16 of SEQ ID NO: 1, a H at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, a H at position P36 of SEQ ID NO: 1, a Q at position V93 of SEQ ID NO: 1, and a F at position R97 of SEQ ID NO: 1.

### I16L_P36H_V93Q_R97W

In some embodiments, a KOD polymerase variant comprises: a L at a position corresponding to I16 of SEQ ID NO: 1, a H at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, a Q at position V93 of SEQ ID NO: 1, and a W at position R97 of SEQ ID NO: 1. In some embodiments, a KOD polymerase variant comprises: a L at position I16 of SEQ ID NO: 1, a H at position P36 of SEQ ID NO: 1, a Q at position V93 of SEQ ID NO: 1, and a W at position R97 of SEQ ID NO: 1.

### I16L_P36H_V93R

In some embodiments, a KOD polymerase variant comprises: a L at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, and a R at position V93 of SEQ ID NO: 1. In some embodiments, a KOD polymerase variant comprises: a L at position I16 of SEQ ID NO: 1, a H at position P36 of SEQ ID NO: 1, and a R at position V93 of SEQ ID NO: 1.

### I16L_P36H_V93R_R97A

In some embodiments, a KOD polymerase variant comprises: a L at a position corresponding to I16 of SEQ ID NO: 1, a H at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a H at position P36 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a H at position P36 of SEQ ID NO: 1, a R at position V93 of SEQ ID NO: 1, and an A at position R97 of SEQ ID NO: 1.

### I16L_P36R

In some embodiments, a KOD polymerase variant comprises: a L at a position corresponding to I16 of SEQ ID NO: 1 and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a R at position P36 of SEQ ID NO: 1. In some embodiments, a KOD polymerase variant comprises: a L at position I16 of SEQ ID NO: 1, and a R at position P36 of SEQ ID NO: 1.

### I16L_P36R_R97A

In some embodiments, a KOD polymerase variant comprises: a L at a position corresponding to I16 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position P36 of SEQ ID NO: 1, and an A at position R97 of SEQ ID NO: 1. In some embodiments, a KOD polymerase variant comprises: a L at position I16 of SEQ ID NO: 1, a R at position P36 of SEQ ID NO: 1, and an A at position R97 of SEQ ID NO: 1.

### I16L_P36R_R97W

In some embodiments, a KOD polymerase variant comprises: a L at a position corresponding to I16 of SEQ ID NO: 1, a R at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position P36 of SEQ ID NO: 1, and a W at position R97 of SEQ ID NO: 1. In some embodiments, a KOD polymerase variant comprises: a L at position I16 of SEQ ID NO: 1, a R at position P36 of SEQ ID NO: 1, and a W at position R97 of SEQ ID NO: 1.

### I16L_P36R_V93I

In some embodiments, a KOD polymerase variant comprises: a L at a position corresponding to I16 of SEQ ID NO: 1, a R at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position P36 of SEQ ID NO: 1, and an I at position V93 of SEQ ID NO: 1. In some embodiments, a KOD polymerase variant comprises: a L at position I16 of SEQ ID NO: 1, a R at position P36 of SEQ ID NO: 1, and an I at position V93 of SEQ ID NO: 1.

### I16L_P36R_V93I_R97H

In some embodiments, a KOD polymerase variant comprises: a L at a position corresponding to I16 of SEQ ID NO: 1, a R at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position P36 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a R at position P36 of SEQ ID NO: 1, an I at position V93 of SEQ ID NO: 1, and a H at position R97 of SEQ ID NO: 1.

### I16L_P36R_V93K

In some embodiments, a KOD polymerase variant comprises: a L at a position corresponding to I16 of SEQ ID NO: 1, a R at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position P36 of SEQ ID NO: 1, and a K at position V93 of SEQ ID NO: 1. In some embodiments, a KOD polymerase variant comprises: a L at position I16 of SEQ ID NO: 1, a R at position P36 of SEQ ID NO: 1, and a K at position V93 of SEQ ID NO: 1.

### I16L_P36S_P90F_V93I

In some embodiments, a KOD polymerase variant comprises: a L at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a L at position I16 of SEQ ID NO: 1, a S at position P36 of SEQ ID NO: 1, a F at position P90 of SEQ ID NO: 1, and an I at position V93 of SEQ ID NO: 1.

### I16L_P36S_P90F_V93I_R97A

In some embodiments, a KOD polymerase variant comprises: a L at a position corresponding to I16 of SEQ ID NO: 1, a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1.

### I16L_P36S_V93E

In some embodiments, a KOD polymerase variant comprises: a L at a position corresponding to I16 of SEQ ID NO: 1, a S at a position corresponding to P36 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, and an E at position V93 of SEQ ID NO: 1. In some embodiments, a KOD polymerase variant comprises: a L at position I16 of SEQ ID NO: 1, a S at position P36 of SEQ ID NO: 1, and an E at position V93 of SEQ ID NO: 1.

### I16L_P36S_V93I_R97F

In some embodiments, a KOD polymerase variant comprises: a L at a position corresponding to I16 of SEQ ID NO: 1, a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a S at position P36 of SEQ ID NO: 1, an I at position V93 of SEQ ID NO: 1, and a F at position R97 of SEQ ID NO: 1.

### I16L_P36S_V93I_R97H

In some embodiments, a KOD polymerase variant comprises: a L at a position corresponding to I16 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a S at position P36 of SEQ ID NO: 1, an I at position V93 of SEQ ID NO: 1, and a H at position R97 of SEQ ID NO: 1.

### I16L_P36S_V93K_R97A

In some embodiments, a KOD polymerase variant comprises: a L at a position corresponding to I16 of SEQ ID NO: 1, a S at a position corresponding to P36 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, a K at position V93 of SEQ ID NO: 1, and an A at position R97 of SEQ ID NO: 1. In some embodiments, a KOD polymerase variant comprises: a L at position I16 of SEQ ID NO: 1, a S at position P36 of SEQ ID NO: 1, a K at position V93 of SEQ ID NO: 1, and an A at position R97 of SEQ ID NO:1.

### I16L_P36S_V93Q_R97A

In some embodiments, a KOD polymerase variant comprises: a L at a position corresponding to I16 of SEQ ID NO: 1, a S at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, a S at position P36 of SEQ ID NO: 1, a Q at position V93 of SEQ ID NO: 1, and an A at position R97 of SEQ ID NO: 1.

### I16L_P36S_V93R

In some embodiments, a KOD polymerase variant comprises: a L at a position corresponding to I16 of SEQ ID NO: 1, a S at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, and a R at position V93 of SEQ ID NO: 1. In some embodiments, a KOD polymerase variant comprises: a L at position I16 of SEQ ID NO: 1, a S at position P36 of SEQ ID NO: 1, and a R at position V93 of SEQ ID NO: 1.

### I16L_P36S_V93R_R97W

In some embodiments, a KOD polymerase variant comprises: a L at a position corresponding to I16 of SEQ ID NO: 1, a S at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P36 of SEQ ID NO: 1, a R at position V93 of SEQ ID NO: 1, and a W at position R97 of SEQ ID NO: 1. In some embodiments, a KOD polymerase variant comprises: a L at position I16 of SEQ ID NO: 1, a S at position P36 of SEQ ID NO: 1, a R at position V93 of SEQ ID NO: 1, and a W at position R97 of SEQ ID NO:1.

### I16L_P36W

In some embodiments, a KOD polymerase variant comprises: a L at a position corresponding to I16 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a W at position P36 of SEQ ID NO: 1. In some embodiments, a KOD polymerase variant comprises: a L at position I16 of SEQ ID NO: 1, and a W at position P36 of SEQ ID NO: 1.

### I16L_P36W_P90F

In some embodiments, a KOD polymerase variant comprises: a L at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and a F at position P90 of SEQ ID NO: 1. In some embodiments, a KOD polymerase variant comprises: a L at position I16 of SEQ ID NO: 1, a W at position P36 of SEQ ID NO: 1, and a F at position P90 of SEQ ID NO: 1.

### I16L_P36W_P90F_R97A

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, and an A at position R97 of SEQ ID NO: 1.

### I16L_P36W_P90F_V93I

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, and an I at position V93 of SEQ ID NO: 1.

### I16L_P36W_P90F_V93I_R97A

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### I16L_P36W_P90F_V93I_R97F

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### I16L_P36W_P90F_V93I_R97H

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### I16L_P36W_P90F_V93I_R97W

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1.

### I16L_P36W_P90F_V93R_R97A

In some embodiments, a KOD polymerase variant comprises: a L at a position corresponding to I16 of SEQ ID NO: 1, a W at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1.

### I16L_P36W_P90I

In some embodiments, a KOD polymerase variant comprises: a L at a position corresponding to I16 of SEQ ID NO: 1, a W at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and an I at position P90 of SEQ ID NO: 1. In some embodiments, a KOD polymerase variant comprises: a L at position I16 of SEQ ID NO: 1, a W at position P36 of SEQ ID NO: 1, and an I at position P90 of SEQ ID NO: 1.

### I16L_P36W_P90I_V93I

In some embodiments, a KOD polymerase variant comprises: a L at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a L at position I16 of SEQ ID NO: 1, a W at position P36 of SEQ ID NO: 1, an I at position P90 of SEQ ID NO: 1, and an I at position V93 of SEQ ID NO: 1.

### I16L_P36W_P90I_V93I_R97A

In some embodiments, a KOD polymerase variant comprises: a L at a position corresponding to I16 of SEQ ID NO: 1, a W at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1.

### I16L_P36W_P90W

In some embodiments, a KOD polymerase variant comprises: a L at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and a W at position P90 of SEQ ID NO: 1. In some embodiments, a KOD polymerase variant comprises: a L at position I16 of SEQ ID NO: 1, a W at position P36 of SEQ ID NO: 1, and a W at position P90 of SEQ ID NO: 1.

### I16L_P36W_P90W_R97A

In some embodiments, a KOD polymerase variant comprises: a L at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a W at position P90 of SEQ ID NO: 1, and an A at position R97 of SEQ ID NO: 1.

### I16L_P36W_P90W_V93I

In some embodiments, a KOD polymerase variant comprises: a L at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a L at position I16 of SEQ ID NO: 1, a W at position P36 of SEQ ID NO: 1, a W at position P90 of SEQ ID NO: 1, and an I at position V93 of SEQ ID NO: 1.

### I16L_P36W_P90W_V93I_R97A

In some embodiments, a KOD polymerase variant comprises: a L at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### I16L_P36W_P90Y_V93R_R97A

In some embodiments, a KOD polymerase variant comprises: a L at a position corresponding to I16 of SEQ ID NO: 1, a W at a position corresponding to P36 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a Y at position P90 of SEQ ID NO: 1, a R at position V93 of SEQ ID NO: 1, and an A at position R97 of SEQ ID NO: 1.

### I16L_P36W_R97A

In some embodiments, a KOD polymerase variant comprises: a L at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, and an A at position R97 of SEQ ID NO: 1.

### I16L_P36W_R97W

In some embodiments, a KOD polymerase variant comprises: a L at a position corresponding to I16 of SEQ ID NO: 1, a W at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and a W at position R97 of SEQ ID NO: 1. In some embodiments, a KOD polymerase variant comprises: a L at position I16 of SEQ ID NO: 1, a W at position P36 of SEQ ID NO: 1, and a W at position R97 of SEQ ID NO: 1.

### I16L_P36W_V93I

In some embodiments, a KOD polymerase variant comprises: a L at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and an I at position V93 of SEQ ID NO: 1. In some embodiments, a KOD polymerase variant comprises: a L at position I16 of SEQ ID NO: 1, a W at position P36 of SEQ ID NO: 1, and an I at position V93 of SEQ ID NO: 1.

### I16L_P36W_V93I_R97A

In some embodiments, a KOD polymerase variant comprises: a L at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a L at position 116 of SEQ ID NO: 1, a W at position P36 of SEQ ID NO: 1, an I at position V93 of SEQ ID NO: 1, and an A at position R97 of SEQ ID NO: 1.

### I16L_P36W_V93I_R97F

In some embodiments, a KOD polymerase variant comprises: a L at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a W at position P36 of SEQ ID NO: 1, an I at position V93 of SEQ ID NO: 1, and a F at position R97 of SEQ ID NO: 1.

### I16L_P36W_V93I_R97H

In some embodiments, a KOD polymerase variant comprises: a L at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a W at position P36 of SEQ ID NO: 1, an I at position V93 of SEQ ID NO: 1, and a H at position R97 of SEQ ID NO: 1.

### I16L_P36W_V93I_R97W

In some embodiments, a KOD polymerase variant comprises: a L at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a W at position P36 of SEQ ID NO: 1, an I at position V93 of SEQ ID NO: 1, and a W at position R97 of SEQ ID NO: 1.

### I16L_P36W_V93Q

In some embodiments, a KOD polymerase variant comprises: a L at a position corresponding to I16 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, and a Q at position V93 of SEQ ID NO: 1. In some embodiments, a KOD polymerase variant comprises: a L at position I16 of SEQ ID NO: 1, a W at position P36 of SEQ ID NO: 1, and a Q at position V93 of SEQ ID NO: 1.

### I16L_P36W_V93R_R97A

In some embodiments, a KOD polymerase variant comprises: a L at a position corresponding to I16 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a W at position P36 of SEQ ID NO: 1, a R at position V93 of SEQ ID NO: 1, and an A at position R97 of SEQ ID NO: 1.

### I16L_P90F

In some embodiments, a KOD polymerase variant comprises: a L at a position corresponding to I16 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a F at position P90 of SEQ ID NO: 1. In some embodiments, a KOD polymerase variant comprises: a L at position I16 of SEQ ID NO: 1, and a F at position P90 of SEQ ID NO: 1.

### I16L_P90F_R97A

In some embodiments, a KOD polymerase variant comprises: a L at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and an A at position R97 of SEQ ID NO: 1. In some embodiments, a KOD polymerase variant comprises: a L at position I16 of SEQ ID NO: 1, a F at position P90 of SEQ ID NO: 1, and an A at position R97 of SEQ ID NO: 1.

### I16L_P90F_R97F

In some embodiments, a KOD polymerase variant comprises: a L at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a F at position R97 of SEQ ID NO: 1. In some embodiments, a KOD polymerase variant comprises: a L at position I16 of SEQ ID NO: 1, a F at position P90 of SEQ ID NO: 1, and a F at position R97 of SEQ ID NO: 1.

### I16L_P90F_R97H

In some embodiments, a KOD polymerase variant comprises: a L at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a H at position R97 of SEQ ID NO: 1. In some embodiments, a KOD polymerase variant comprises: a L at position I16 of SEQ ID NO: 1, a F at position P90 of SEQ ID NO: 1, and a H at position R97 of SEQ ID NO: 1.

### I16L_P90F_R97W

In some embodiments, a KOD polymerase variant comprises: a L at a position corresponding to I16 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a W at position R97 of SEQ ID NO: 1. In some embodiments, a KOD polymerase variant comprises: a L at position I16 of SEQ ID NO: 1, a F at position P90 of SEQ ID NO: 1, and a W at position R97 of SEQ ID NO: 1.

### I16L_P90F_R119Q

In some embodiments, a KOD polymerase variant comprises: a L at a position corresponding to I16 of SEQ ID NO: 1, a F at a position corresponding to P90 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a Q at position R119 of SEQ ID NO: 1. In some embodiments, a KOD polymerase variant comprises: a L at position I16 of SEQ ID NO: 1, a F at position P90 of SEQ ID NO: 1, and a Q at position R119 of SEQ ID NO: 1.

### I16L_P90F_V93I

In some embodiments, a KOD polymerase variant comprises: a L at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and an I at position V93 of SEQ ID NO: 1. In some embodiments, a KOD polymerase variant comprises: a L at position I16 of SEQ ID NO: 1, a F at position P90 of SEQ ID NO: 1, and an I at position V93 of SEQ ID NO: 1.

### I16L_P90F_V93I_R97A

In some embodiments, a KOD polymerase variant comprises: a L at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a F at position P90 of SEQ ID NO: 1, an I at position V93 of SEQ ID NO: 1, and an A at position R97 of SEQ ID NO: 1.

### I16L_P90F_V93I_R97F

In some embodiments, a KOD polymerase variant comprises: a L at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a F at position P90 of SEQ ID NO: 1, an I at position V93 of SEQ ID NO: 1, and a F at position R97 of SEQ ID NO: 1.

### I16L_P90F_V93I_R97H

In some embodiments, a KOD polymerase variant comprises: a L at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a F at position P90 of SEQ ID NO: 1, an I at position V93 of SEQ ID NO: 1, and a H at position R97 of SEQ ID NO: 1.

### I16L_P90F_V93I_R97W

In some embodiments, a KOD polymerase variant comprises: a L at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a F at position P90 of SEQ ID NO: 1, an I at position V93 of SEQ ID NO: 1, and a W at position R97 of SEQ ID NO: 1.

### I16L_P90F_V93Q

In some embodiments, a KOD polymerase variant comprises: a L at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a Q at position V93 of SEQ ID NO: 1. In some embodiments, a KOD polymerase variant comprises: a L at position I16 of SEQ ID NO: 1, a F at position P90 of SEQ ID NO: 1, and a Q at position V93 of SEQ ID NO: 1.

### I16L_P90F_V93Q_R97A

In some embodiments, a KOD polymerase variant comprises: a L at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, a F at position P90 of SEQ ID NO: 1, a Q at position V93 of SEQ ID NO: 1, and an A at position R97 of SEQ ID NO: 1.

### I16L_P90F_V93Q_R97F

In some embodiments, a KOD polymerase variant comprises: a L at a position corresponding to I16 of SEQ ID NO: 1, a F at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, a F at position P90 of SEQ ID NO: 1, a Q at position V93 of SEQ ID NO: 1, and a F at position R97 of SEQ ID NO: 1.

### I16L_P90F_V93R

In some embodiments, a KOD polymerase variant comprises: a L at a position corresponding to I16 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, and a R at position V93 of SEQ ID NO: 1. In some embodiments, a KOD polymerase variant comprises: a L at position I16 of SEQ ID NO: 1, a F at position P90 of SEQ ID NO: 1, and a R at position V93 of SEQ ID NO: 1.

### I16L_P90F_V93R_R97F

In some embodiments, a KOD polymerase variant comprises: a L at a position corresponding to I16 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: a L at position I16 of SEQ ID NO: 1, a F at position P90 of SEQ ID NO: 1, a R at position V93 of SEQ ID NO: 1, and a F at position R97 of SEQ ID NO: 1.

### I16L_P90I

In some embodiments, a KOD polymerase variant comprises: a L at a position corresponding to I16 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and an I at position P90 of SEQ ID NO: 1. In some embodiments, a KOD polymerase variant comprises: a L at position I16 of SEQ ID NO: 1, and an I at position P90 of SEQ ID NO: 1.

### I16L_P90I_R97A

In some embodiments, a KOD polymerase variant comprises: a L at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and an A at position R97 of SEQ ID NO: 1. In some embodiments, a KOD polymerase variant comprises: a L at position I16 of SEQ ID NO: 1, an I at position P90 of SEQ ID NO: 1, and an A at position R97 of SEQ ID NO: 1.

### I16L_P90I_R97F

In some embodiments, a KOD polymerase variant comprises: a L at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a F at position R97 of SEQ ID NO: 1. In some embodiments, a KOD polymerase variant comprises: a L at position I16 of SEQ ID NO: 1, an I at position P90 of SEQ ID NO: 1, and a F at position R97 of SEQ ID NO: 1.

### I16L_P90I_R97H

In some embodiments, a KOD polymerase variant comprises: a L at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a H at position R97 of SEQ ID NO: 1. In some embodiments, a KOD polymerase variant comprises: a L at position I16 of SEQ ID NO: 1, an I at position P90 of SEQ ID NO: 1, and a H at position R97 of SEQ ID NO: 1.

### I16L_P90I_R97W

In some embodiments, a KOD polymerase variant comprises: a L at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a W at position R97 of SEQ ID NO: 1. In some embodiments, a KOD polymerase variant comprises: a L at position I16 of SEQ ID NO: 1, an I at position P90 of SEQ ID NO: 1, and a W at position R97 of SEQ ID NO: 1.

### I16L_P90I_V93I

In some embodiments, a KOD polymerase variant comprises: a L at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and an I at position V93 of SEQ ID NO: 1. In some embodiments, a KOD polymerase variant comprises: a L at position I16 of SEQ ID NO: 1, an I at position P90 of SEQ ID NO: 1, and an I at position V93 of SEQ ID NO: 1.

### I16L_P90I_V93I_R97A

In some embodiments, a KOD polymerase variant comprises: a L at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, an I at position P90 of SEQ ID NO: 1, an I at position V93 of SEQ ID NO: 1, and an A at position R97 of SEQ ID NO: 1.

### I16L_P90I_V93I_R97F

In some embodiments, a KOD polymerase variant comprises: a L at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, an I at position P90 of SEQ ID NO: 1, an I at position V93 of SEQ ID NO: 1, and a F at position R97 of SEQ ID NO: 1.

### I16L_P90I_V93I_R97H

In some embodiments, a KOD polymerase variant comprises: a L at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, an I at position P90 of SEQ ID NO: 1, an I at position V93 of SEQ ID NO: 1, and a H at position R97 of SEQ ID NO: 1.

### I16L_P90I_V93I_R97W

In some embodiments, a KOD polymerase variant comprises: a L at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, an I at position P90 of SEQ ID NO: 1, an I at position V93 of SEQ ID NO: 1, and a W at position R97 of SEQ ID NO: 1.

### I16L_P90I_V93K

In some embodiments, a KOD polymerase variant comprises: a L at a position corresponding to I16 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a K at position V93 of SEQ ID NO: 1. In some embodiments, a KOD polymerase variant comprises: a L at position I16 of SEQ ID NO: 1, an I at position P90 of SEQ ID NO: 1, and a K at position V93 of SEQ ID NO: 1.

### I16L_P90I_V93K_R97F

In some embodiments, a KOD polymerase variant comprises: a L at a position corresponding to I16 of SEQ ID NO: 1, an I at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a K at position V93 of SEQ ID NO: 1, and a F at position R97 of SEQ ID NO: 1. In some embodiments, a KOD polymerase variant comprises: a L at position I16 of SEQ ID NO: 1, an I at position P90 of SEQ ID NO: 1, a K at position V93 of SEQ ID NO: 1, and a F at position R97 of SEQ ID NO: 1.

### I16L_P90I_V93K_R97W

In some embodiments, a KOD polymerase variant comprises: a L at a position corresponding to I16 of SEQ ID NO: 1, an I at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a K at position V93 of SEQ ID NO: 1, and a W at position R97 of SEQ ID NO: 1. In some embodiments, a KOD polymerase variant comprises: a L at position I16 of SEQ ID NO: 1, an I at position P90 of SEQ ID NO: 1, a K at position V93 of SEQ ID NO: 1, and a W at position R97 of SEQ ID NO: 1.

### I16L_P90I_V93Q

In some embodiments, a KOD polymerase variant comprises: a L at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a Q at position V93 of SEQ ID NO: 1. In some embodiments, a KOD polymerase variant comprises: a L at position I16 of SEQ ID NO: 1, an I at position P90 of SEQ ID NO: 1, and a Q at position V93 of SEQ ID NO: 1.

### I16L_P90I_V93Q_R97A

In some embodiments, a KOD polymerase variant comprises: a L at a position corresponding to I16 of SEQ ID NO: 1, an I at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, an I at position P90 of SEQ ID NO: 1, a Q at position V93 of SEQ ID NO: 1, and an A at position R97 of SEQ ID NO: 1.

### I16L_P90I_V93Q_R97F

In some embodiments, a KOD polymerase variant comprises: a L at a position corresponding to I16 of SEQ ID NO: 1, an I at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, an I at position P90 of SEQ ID NO: 1, a Q at position V93 of SEQ ID NO: 1, and a F at position R97 of SEQ ID NO: 1.

### I16L_P90I_V93R

In some embodiments, a KOD polymerase variant comprises: a L at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, and a R at position V93 of SEQ ID NO: 1. In some embodiments, a KOD polymerase variant comprises: a L at position I16 of SEQ ID NO: 1, an I at position P90 of SEQ ID NO: 1, and a R at position V93 of SEQ ID NO: 1.

### I16L_P90I_V93R_R97A

In some embodiments, a KOD polymerase variant comprises: a L at a position corresponding to I16 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, an I at position P90 of SEQ ID NO: 1, a R at position V93 of SEQ ID NO: 1, and an A at position R97 of SEQ ID NO: 1.

### I16L_P90I_V93R_R97F

In some embodiments, a KOD polymerase variant comprises: a L at a position corresponding to I16 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a L at position I16 of SEQ ID NO: 1, an I at position P90 of SEQ ID NO: 1, a R at position V93 of SEQ ID NO: 1, and a F at position R97 of SEQ ID NO: 1.

### I16L_P90S

In some embodiments, a KOD polymerase variant comprises: a L at a position corresponding to I16 of SEQ ID NO: 1 and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a S at position P90 of SEQ ID NO: 1. In some embodiments, a KOD polymerase variant comprises: a L at position I16 of SEQ ID NO: 1, and a S at position P90 of SEQ ID NO: 1.

### I16L_P90S_V93I_R97H

In some embodiments, a KOD polymerase variant comprises: a L at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a S at position P90 of SEQ ID NO: 1, an I at position V93 of SEQ ID NO: 1, and a H at position R97 of SEQ ID NO: 1.

### I16L_P90S_V93I_R97W

In some embodiments, a KOD polymerase variant comprises: a L at a position corresponding to I16 of SEQ ID NO: 1, a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a S at position P90 of SEQ ID NO: 1, an I at position V93 of SEQ ID NO: 1, and a W at position R97 of SEQ ID NO: 1.

### I16L_P90S_V93Q

In some embodiments, a KOD polymerase variant comprises: a L at a position corresponding to I16 of SEQ ID NO: 1, a S at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P90 of SEQ ID NO: 1, and a Q at position V93 of SEQ ID NO: 1. In some embodiments, a KOD polymerase variant comprises: a L at position I16 of SEQ ID NO: 1, a S at position P90 of SEQ ID NO: 1, and a Q at position V93 of SEQ ID NO: 1.

### I16L_P90S_V93R

In some embodiments, a KOD polymerase variant comprises: a L at a position corresponding to I16 of SEQ ID NO: 1, a S at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a S at position P90 of SEQ ID NO: 1, and a R at position V93 of SEQ ID NO: 1. In some embodiments, a KOD polymerase variant comprises: a L at position I16 of SEQ ID NO: 1, a S at position P90 of SEQ ID NO: 1, and a R at position V93 of SEQ ID NO: 1.

### I16L_P90W

In some embodiments, a KOD polymerase variant comprises: a L at a position corresponding to I16 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a W at position P90 of SEQ ID NO: 1. In some embodiments, a KOD polymerase variant comprises: a L at position I16 of SEQ ID NO: 1, and a W at position P90 of SEQ ID NO: 1.

### I16L_P90W_R97A

In some embodiments, a KOD polymerase variant comprises: a L at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, and an A at position R97 of SEQ ID NO: 1. In some embodiments, a KOD polymerase variant comprises: a L at position I16 of SEQ ID NO: 1, a W at position P90 of SEQ ID NO: 1, and an A at position R97 of SEQ ID NO: 1.

### I16L_P90W_R97F

In some embodiments, a KOD polymerase variant comprises: a L at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, and a F at position R97 of SEQ ID NO: 1. In some embodiments, a KOD polymerase variant comprises: a L at position I16 of SEQ ID NO: 1, a W at position P90 of SEQ ID NO: 1, and a F at position R97 of SEQ ID NO: 1.

### I16L_P90W_R97H

In some embodiments, a KOD polymerase variant comprises: a L at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, and a H at position R97 of SEQ ID NO: 1. In some embodiments, a KOD polymerase variant comprises: a L at position I16 of SEQ ID NO: 1, a W at position P90 of SEQ ID NO: 1, and a H at position R97 of SEQ ID NO: 1.

### I16L_P90W_V93E_R97H

In some embodiments, a KOD polymerase variant comprises: a L at a position corresponding to I16 of SEQ ID NO: 1, a W at a position corresponding to P90 of SEQ ID NO: 1, an E at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, an E at position V93 of SEQ ID NO: 1, and a H at position R97 of SEQ ID NO: 1. In some embodiments, a KOD polymerase variant comprises: a L at position I16 of SEQ ID NO: 1, a W at position P90 of SEQ ID NO: 1, an E at position V93 of SEQ ID NO: 1, and a H at position R97 of SEQ ID NO: 1.

### I16L_P90W_V93I

In some embodiments, a KOD polymerase variant comprises: a L at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, and an I at position V93 of SEQ ID NO: 1. In some embodiments, a KOD polymerase variant comprises: a L at position I16 of SEQ ID NO: 1, a W at position P90 of SEQ ID NO: 1, and an I at position V93 of SEQ ID NO: 1.

### I16L_P90W_V93I_R97A

In some embodiments, a KOD polymerase variant comprises: a L at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a W at position P90 of SEQ ID NO: 1, an I at position V93 of SEQ ID NO: 1, and an A at position R97 of SEQ ID NO: 1.

### I16L_P90W_V93I_R97F

In some embodiments, a KOD polymerase variant comprises: a L at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a W at position P90 of SEQ ID NO: 1, an I at position V93 of SEQ ID NO: 1, and a F at position R97 of SEQ ID NO: 1.

### I16L_P90W_V93I_R97H

In some embodiments, a KOD polymerase variant comprises: a L at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a W at position P90 of SEQ ID NO: 1, an I at position V93 of SEQ ID NO: 1, and a H at position R97 of SEQ ID NO: 1.

### I16L_P90W_V93I_R97W

In some embodiments, a KOD polymerase variant comprises: a L at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a W at position P90 of SEQ ID NO: 1, an I at position V93 of SEQ ID NO: 1, and a W at position R97 of SEQ ID NO: 1.

### I16L_P90W_V93Q

In some embodiments, a KOD polymerase variant comprises: a L at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, and a Q at position V93 of SEQ ID NO: 1. In some embodiments, a KOD polymerase variant comprises: a L at position I16 of SEQ ID NO: 1, a W at position P90 of SEQ ID NO: 1, and a Q at position V93 of SEQ ID NO: 1.

### I16L_P90W_V93Q_R97A

In some embodiments, a KOD polymerase variant comprises: a L at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, a W at position P90 of SEQ ID NO: 1, a Q at position V93 of SEQ ID NO: 1, and an A at position R97 of SEQ ID NO: 1.

### I16L_P90W_V93Q_R97F

In some embodiments, a KOD polymerase variant comprises: a L at a position corresponding to I16 of SEQ ID NO: 1, a W at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, a W at position P90 of SEQ ID NO: 1, a Q at position V93 of SEQ ID NO: 1, and a F at position R97 of SEQ ID NO: 1.

### I16L_P90W_V93Q_R97H

In some embodiments, a KOD polymerase variant comprises: a L at a position corresponding to I16 of SEQ ID NO: 1, a W at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, a Q at position V93 of SEQ ID NO: 1, and a H at position R97 of SEQ ID NO: 1. In some embodiments, a KOD polymerase variant comprises: a L at position I16 of SEQ ID NO: 1, a W at position P90 of SEQ ID NO: 1, a Q at position V93 of SEQ ID NO: 1, and a H at position R97 of SEQ ID NO: 1.

### I16L_P90W_V93R_R97A

In some embodiments, a KOD polymerase variant comprises: a L at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a W at position P90 of SEQ ID NO: 1, a R at position V93 of SEQ ID NO: 1, and an A at position R97 of SEQ ID NO: 1.

### I16L_P90Y

In some embodiments, a KOD polymerase variant comprises: a L at a position corresponding to I16 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a Y at position P90 of SEQ ID NO: 1. In some embodiments, a KOD polymerase variant comprises: a L at position I16 of SEQ ID NO: 1, and a Y at position P90 of SEQ ID NO: 1.

### I16L_P90Y_R97A

In some embodiments, a KOD polymerase variant comprises: a L at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and an A at position R97 of SEQ ID NO: 1. In some embodiments, a KOD polymerase variant comprises: a L at position I16 of SEQ ID NO: 1, a Y at position P90 of SEQ ID NO: 1, and an A at position R97 of SEQ ID NO: 1.

### I16L_P90Y_R97F

In some embodiments, a KOD polymerase variant comprises: a L at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and a F at position R97 of SEQ ID NO: 1. In some embodiments, a KOD polymerase variant comprises: a L at position I16 of SEQ ID NO: 1, a Y at position P90 of SEQ ID NO: 1, and a F at position R97 of SEQ ID NO: 1.

### I16L_P90Y_R97H

In some embodiments, a KOD polymerase variant comprises: a L at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and a H at position R97 of SEQ ID NO: 1. In some embodiments, a KOD polymerase variant comprises: a L at position I16 of SEQ ID NO: 1, a Y at position P90 of SEQ ID NO: 1, and a H at position R97 of SEQ ID NO: 1.

### I16L_P90Y_R97W

In some embodiments, a KOD polymerase variant comprises: a L at a position corresponding to I16 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and a W at position R97 of SEQ ID NO: 1. In some embodiments, a KOD polymerase variant comprises: a L at position I16 of SEQ ID NO: 1, a Y at position P90 of SEQ ID NO: 1, and a W at position R97 of SEQ ID NO: 1.

### I16L_P90Y_V93I

In some embodiments, a KOD polymerase variant comprises: a L at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and an I at position V93 of SEQ ID NO: 1. In some embodiments, a KOD polymerase variant comprises: a L at position I16 of SEQ ID NO: 1, a Y at position P90 of SEQ ID NO: 1, and an I at position V93 of SEQ ID NO: 1.

### I16L_P90Y_V93I_R97A

In some embodiments, a KOD polymerase variant comprises: a L at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, a Y at position P90 of SEQ ID NO: 1, an I at position V93 of SEQ ID NO: 1, and an A at position R97 of SEQ ID NO: 1.

### I16L_P90Y_V93I_R97F

In some embodiments, a KOD polymerase variant comprises: a L at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, a Y at position P90 of SEQ ID NO: 1, an I at position V93 of SEQ ID NO: 1, and a F at position R97 of SEQ ID NO: 1.

### I16L_P90Y_V93I_R97H

In some embodiments, a KOD polymerase variant comprises: a L at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position I16 of SEQ ID NO: 1, a Y at position P90 of SEQ ID NO: 1, an I at position V93 of SEQ ID NO: 1, and a H at position R97 of SEQ ID NO:1.

### I16L_P90Y_V93I_R97W

In some embodiments, a KOD polymerase variant comprises: a L at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, a Y at position P90 of SEQ ID NO: 1, an I at position V93 of SEQ ID NO: 1, and a W at position R97 of SEQ ID NO: 1.

### I16L_P90Y_V93K_R97A

In some embodiments, a KOD polymerase variant comprises: a L at a position corresponding to I16 of SEQ ID NO: 1, a Y at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, a K at position V93 of SEQ ID NO: 1, and an A at position R97 of SEQ ID NO: 1. In some embodiments, a KOD polymerase variant comprises: a L at position I16 of SEQ ID NO: 1, a Y at position P90 of SEQ ID NO: 1, a K at position V93 of SEQ ID NO: 1, and an A at position R97 of SEQ ID NO: 1.

### I16L_P90Y_V93K_R97F

In some embodiments, a KOD polymerase variant comprises: a L at a position corresponding to I16 of SEQ ID NO: 1, a Y at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, a K at position V93 of SEQ ID NO: 1, and a F at position R97 of SEQ ID NO: 1. In some embodiments, a KOD polymerase variant comprises: a L at position I16 of SEQ ID NO: 1, a Y at position P90 of SEQ ID NO: 1, a K at position V93 of SEQ ID NO: 1, and a F at position R97 of SEQ ID NO: 1.

### I16L_P90Y_V93Q

In some embodiments, a KOD polymerase variant comprises: a L at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, and a Q at position V93 of SEQ ID NO: 1. In some embodiments, a KOD polymerase variant comprises: a L at position I16 of SEQ ID NO: 1, a Y at position P90 of SEQ ID NO: 1, and a Q at position V93 of SEQ ID NO: 1.

### I16L_P90Y_V93R_R97A

In some embodiments, a KOD polymerase variant comprises: a L at a position corresponding to I16 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a Y at position P90 of SEQ ID NO: 1, a R at position V93 of SEQ ID NO: 1, and an A at position R97 of SEQ ID NO: 1.

### I16L_R97A

In some embodiments, a KOD polymerase variant comprises: a L at a position corresponding to I16 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and an A at position R97 of SEQ ID NO: 1. In some embodiments, a KOD polymerase variant comprises: a L at position I16 of SEQ ID NO: 1, and an A at position R97 of SEQ ID NO: 1.

### I16L_R97F

In some embodiments, a KOD polymerase variant comprises: a L at a position corresponding to I16 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a F at position R97 of SEQ ID NO: 1. In some embodiments, a KOD polymerase variant comprises: a L at position I16 of SEQ ID NO: 1, and a F at position R97 of SEQ ID NO: 1.

### I16L_R97F_R119Q

In some embodiments, a KOD polymerase variant comprises: a L at a position corresponding to I16 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a F at position R97 of SEQ ID NO: 1, and a Q at position R119 of SEQ ID NO: 1. In some embodiments, a KOD polymerase variant comprises: a L at position I16 of SEQ ID NO: 1, a F at position R97 of SEQ ID NO: 1, and a Q at position R119 of SEQ ID NO: 1.

### I16L_R97H

In some embodiments, a KOD polymerase variant comprises: a L at a position corresponding to I16 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a H at position R97 of SEQ ID NO: 1. In some embodiments, a KOD polymerase variant comprises: a L at position I16 of SEQ ID NO: 1, and a H at position R97 of SEQ ID NO: 1.

### I16L_R97W

In some embodiments, a KOD polymerase variant comprises: a L at a position corresponding to I16 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a W at position R97 of SEQ ID NO: 1. In some embodiments, a KOD polymerase variant comprises: a L at position I16 of SEQ ID NO: 1, and a W at position R97 of SEQ ID NO: 1.

### I16L_V93E

In some embodiments, a KOD polymerase variant comprises: a L at a position corresponding to I16 of SEQ ID NO: 1 and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and an E at position V93 of SEQ ID NO: 1. In some embodiments, a KOD polymerase variant comprises: a L at position I16 of SEQ ID NO: 1, and an E at position V93 of SEQ ID NO: 1.

### I16L_V93E_R97F

In some embodiments, a KOD polymerase variant comprises: a L at a position corresponding to I16 of SEQ ID NO: 1, an E at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an E at position V93 of SEQ ID NO: 1, and a F at position R97 of SEQ ID NO: 1. In some embodiments, a KOD polymerase variant comprises: a L at position I16 of SEQ ID NO: 1, an E at position V93 of SEQ ID NO: 1, and a F at position R97 of SEQ ID NO: 1.

### I16L_V93E_R97H

In some embodiments, a KOD polymerase variant comprises: a L at a position corresponding to I16 of SEQ ID NO: 1, an E at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an E at position V93 of SEQ ID NO: 1, and a H at position R97 of SEQ ID NO: 1. In some embodiments, a KOD polymerase variant comprises: a L at position I16 of SEQ ID NO: 1, an E at position V93 of SEQ ID NO: 1, and a H at position R97 of SEQ ID NO: 1.

### I16L_V93I

In some embodiments, a KOD polymerase variant comprises: a L at a position corresponding to I16 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and an I at position V93 of SEQ ID NO: 1. In some embodiments, a KOD polymerase variant comprises: a L at position I16 of SEQ ID NO: 1, and an I at position V93 of SEQ ID NO: 1.

### I16L_V93I_R97A

In some embodiments, a KOD polymerase variant comprises: a L at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, and an A at position R97 of SEQ ID NO: 1. In some embodiments, a KOD polymerase variant comprises: a L at position I16 of SEQ ID NO: 1, an I at position V93 of SEQ ID NO: 1, and an A at position R97 of SEQ ID NO: 1.

### I16L_V93I_R97A_R119Q

In some embodiments, a KOD polymerase variant comprises: a L at a position corresponding to I16 of SEQ ID NO: 1, an I at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, an A at position R97 of SEQ ID NO: 1, and a Q at position R119 of SEQ ID NO: 1. In some embodiments, a KOD polymerase variant comprises: a L at position I16 of SEQ ID NO: 1, an I at position V93 of SEQ ID NO: 1, an A at position R97 of SEQ ID NO: 1, and a Q at position R119 of SEQ ID NO: 1.

### I16L_V93I_R97F

In some embodiments, a KOD polymerase variant comprises: a L at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, and a F at position R97 of SEQ ID NO: 1. In some embodiments, a KOD polymerase variant comprises: a L at position I16 of SEQ ID NO: 1, an I at position V93 of SEQ ID NO: 1, and a F at position R97 of SEQ ID NO: 1.

### I16L_V93I_R97F_R119Q

In some embodiments, a KOD polymerase variant comprises: a L at a position corresponding to I16 of SEQ ID NO: 1, an I at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, a F at position R97 of SEQ ID NO: 1, and a Q at position R119 of SEQ ID NO: 1. In some embodiments, a KOD polymerase variant comprises: a L at position I16 of SEQ ID NO: 1, an I at position V93 of SEQ ID NO: 1, a F at position R97 of SEQ ID NO: 1, and a Q at position R119 of SEQ ID NO: 1.

### I16L_V93I_R97W

In some embodiments, a KOD polymerase variant comprises: a L at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, and a W at position R97 of SEQ ID NO: 1. In some embodiments, a KOD polymerase variant comprises: a L at position I16 of SEQ ID NO: 1, an I at position V93 of SEQ ID NO: 1, and a W at position R97 of SEQ ID NO: 1.

### I16L_V93I_R119Q

In some embodiments, a KOD polymerase variant comprises: a L at a position corresponding to I16 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, an I at position V93 of SEQ ID NO: 1, and a Q at position R119 of SEQ ID NO: 1. In some embodiments, a KOD polymerase variant comprises: a L at position I16 of SEQ ID NO: 1, an I at position V93 of SEQ ID NO: 1, and a Q at position R119 of SEQ ID NO: 1.

### I16L_V93K

In some embodiments, a KOD polymerase variant comprises: a L at a position corresponding to I16 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a K at position V93 of SEQ ID NO: 1. In some embodiments, a KOD polymerase variant comprises: a L at position I16 of SEQ ID NO: 1, and a K at position V93 of SEQ ID NO: 1.

### I16L_V93K_R97A

In some embodiments, a KOD polymerase variant comprises: a L at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and an A at position R97 of SEQ ID NO: 1. In some embodiments, a KOD polymerase variant comprises: a L at position I16 of SEQ ID NO: 1, a K at position V93 of SEQ ID NO: 1, and an A at position R97 of SEQ ID NO: 1.

### I16L_V93K_R97F

In some embodiments, a KOD polymerase variant comprises: a L at a position corresponding to I16 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and a F at position R97 of SEQ ID NO: 1. In some embodiments, a KOD polymerase variant comprises: a L at position I16 of SEQ ID NO: 1, a K at position V93 of SEQ ID NO: 1, and a F at position R97 of SEQ ID NO: 1.

### I16L_V93K_R97H

In some embodiments, a KOD polymerase variant comprises: a L at a position corresponding to I16 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and a H at position R97 of SEQ ID NO: 1. In some embodiments, a KOD polymerase variant comprises: a L at position I16 of SEQ ID NO: 1, a K at position V93 of SEQ ID NO: 1, and a H at position R97 of SEQ ID NO: 1.

### I16L_V93K_R97P

In some embodiments, a KOD polymerase variant comprises: a L at a position corresponding to I16 of SEQ ID NO: 1, a K at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and a P at position R97 of SEQ ID NO: 1. In some embodiments, a KOD polymerase variant comprises: a L at position I16 of SEQ ID NO: 1, a K at position V93 of SEQ ID NO: 1, and a P at position R97 of SEQ ID NO: 1.

### I16L_V93K_R97W

In some embodiments, a KOD polymerase variant comprises: a L at a position corresponding to I16 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and a W at position R97 of SEQ ID NO: 1. In some embodiments, a KOD polymerase variant comprises: a L at position I16 of SEQ ID NO: 1, a K at position V93 of SEQ ID NO: 1, and a W at position R97 of SEQ ID NO: 1.

### I16L_V93K_R119Q

In some embodiments, a KOD polymerase variant comprises: a L at a position corresponding to I16 of SEQ ID NO: 1, a K at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a K at position V93 of SEQ ID NO: 1, and a Q at position R119 of SEQ ID NO: 1. In some embodiments, a KOD polymerase variant comprises: a L at position I16 of SEQ ID NO: 1, a K at position V93 of SEQ ID NO: 1, and a Q at position R119 of SEQ ID NO: 1.

### I16L_V93Q

In some embodiments, a KOD polymerase variant comprises: a L at a position corresponding to I16 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a Q at position V93 of SEQ ID NO: 1. In some embodiments, a KOD polymerase variant comprises: a L at position I16 of SEQ ID NO: 1, and a Q at position V93 of SEQ ID NO: 1.

### I16L_V93Q_R97A

In some embodiments, a KOD polymerase variant comprises: a L at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Q at position V93 of SEQ ID NO: 1, and an A at position R97 of SEQ ID NO: 1. In some embodiments, a KOD polymerase variant comprises: a L at position I16 of SEQ ID NO: 1, a Q at position V93 of SEQ ID NO: 1, and an A at position R97 of SEQ ID NO: 1.

### I16L_V93Q_R97F

In some embodiments, a KOD polymerase variant comprises: a L at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Q at position V93 of SEQ ID NO: 1, and a F at position R97 of SEQ ID NO: 1. In some embodiments, a KOD polymerase variant comprises: a L at position I16 of SEQ ID NO: 1, a Q at position V93 of SEQ ID NO: 1, and a F at position R97 of SEQ ID NO: 1.

### I16L_V93Q_R97H

In some embodiments, a KOD polymerase variant comprises: a L at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Q at position V93 of SEQ ID NO: 1, and a H at position R97 of SEQ ID NO: 1. In some embodiments, a KOD polymerase variant comprises: a L at position I16 of SEQ ID NO: 1, a Q at position V93 of SEQ ID NO: 1, and a H at position R97 of SEQ ID NO: 1.

### I16L_V93Q_R97W

In some embodiments, a KOD polymerase variant comprises: a L at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a Q at position V93 of SEQ ID NO: 1, and a W at position R97 of SEQ ID NO: 1. In some embodiments, a KOD polymerase variant comprises: a L at position I16 of SEQ ID NO: 1, a Q at position V93 of SEQ ID NO: 1, and a W at position R97 of SEQ ID NO: 1.

### I16L_V93R

In some embodiments, a KOD polymerase variant comprises: a L at a position corresponding to I16 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, and a R at position V93 of SEQ ID NO: 1. In some embodiments, a KOD polymerase variant comprises: a L at position I16 of SEQ ID NO: 1, and a R at position V93 of SEQ ID NO: 1.

### I16L_V93R_R97A

In some embodiments, a KOD polymerase variant comprises: a L at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, and an A at position R97 of SEQ ID NO: 1. In some embodiments, a KOD polymerase variant comprises: a L at position I16 of SEQ ID NO: 1, a R at position V93 of SEQ ID NO: 1, and an A at position R97 of SEQ ID NO: 1.

### I16L_V93R_R97A_R119M

In some embodiments, a KOD polymerase variant comprises: a L at a position corresponding to I16 of SEQ ID NO: 1, a R at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, an A at position R97 of SEQ ID NO: 1, and a M at position R119 of SEQ ID NO: 1. In some embodiments, a KOD polymerase variant comprises: a L at position I16 of SEQ ID NO: 1, a R at position V93 of SEQ ID NO: 1, an A at position R97 of SEQ ID NO: 1, and a M at position R119 of SEQ ID NO: 1.

### I16L_V93R_R97A_R119Q

In some embodiments, a KOD polymerase variant comprises: a L at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a L at position I16 of SEQ ID NO: 1, a R at position V93 of SEQ ID NO: 1, an A at position R97 of SEQ ID NO: 1, and a Q at position R119 of SEQ ID NO: 1.

### I16L_V93R_R97F

In some embodiments, a KOD polymerase variant comprises: a L at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, and a F at position R97 of SEQ ID NO: 1. In some embodiments, a KOD polymerase variant comprises: a L at position I16 of SEQ ID NO: 1, a R at position V93 of SEQ ID NO: 1, and a F at position R97 of SEQ ID NO: 1.

### I16L_V93R_R97H

In some embodiments, a KOD polymerase variant comprises: a L at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, and a H at position R97 of SEQ ID NO: 1. In some embodiments, a KOD polymerase variant comprises: a L at position I16 of SEQ ID NO: 1, a R at position V93 of SEQ ID NO: 1, and a H at position R97 of SEQ ID NO: 1.

### I16L_V93R_R97W

In some embodiments, a KOD polymerase variant comprises: a L at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, and a W at position R97 of SEQ ID NO: 1. In some embodiments, a KOD polymerase variant comprises: a L at position I16 of SEQ ID NO: 1, a R at position V93 of SEQ ID NO: 1, and a W at position R97 of SEQ ID NO: 1.

### I16L_V93R_R97W_R119Q

In some embodiments, a KOD polymerase variant comprises: a L at a position corresponding to I16 of SEQ ID NO: 1, a R at a position corresponding to V93 of SEQ ID NO: 1, a W at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, a W at position R97 of SEQ ID NO: 1, and a Q at position R119 of SEQ ID NO: 1. In some embodiments, a KOD polymerase variant comprises: a L at position I16 of SEQ ID NO: 1, a R at position V93 of SEQ ID NO: 1, a W at position R97 of SEQ ID NO: 1, and a Q at position R119 of SEQ ID NO: 1.

### I16L_V93R_R119Q

In some embodiments, a KOD polymerase variant comprises: a L at a position corresponding to I16 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position I16 of SEQ ID NO: 1, a R at position V93 of SEQ ID NO: 1, and a Q at position R119 of SEQ ID NO: 1. In some embodiments, a KOD polymerase variant comprises: a L at position I16 of SEQ ID NO: 1, a R at position V93 of SEQ ID NO: 1, and a Q at position R119 of SEQ ID NO: 1.

### I16M_P36A_P90F

In some embodiments, a KOD polymerase variant comprises: a M at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a F at position P90 of SEQ ID NO: 1. In some embodiments, a KOD polymerase variant comprises: a M at position I16 of SEQ ID NO: 1, an A at position P36 of SEQ ID NO: 1, and a F at position P90 of SEQ ID NO: 1.

### I16M_P36A_P90F_R97A

In some embodiments, a KOD polymerase variant comprises: a M at a position corresponding to I16 of SEQ ID NO: 1, an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a F at position P90 of SEQ ID NO: 1, and an A at position R97 of SEQ ID NO: 1. In some embodiments, a KOD polymerase variant comprises: a M at position I16 of SEQ ID NO: 1, an A at position P36 of SEQ ID NO: 1, a F at position P90 of SEQ ID NO: 1, and an A at position R97 of SEQ ID NO: 1.

### I16M_P36A_P90F_V93I

In some embodiments, a KOD polymerase variant comprises: a M at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a M at position I16 of SEQ ID NO: 1, an A at position P36 of SEQ ID NO: 1, a F at position P90 of SEQ ID NO: 1, and an I at position V93 of SEQ ID NO: 1.

### I16M_P36A_P90F_V93I_R97A

In some embodiments, a KOD polymerase variant comprises: a M at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1.

### I16M_P36A_P90F_V93I_R97F

In some embodiments, a KOD polymerase variant comprises: a M at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1.

### I16M_P36A_P90F_V93I_R97H

In some embodiments, a KOD polymerase variant comprises: a M at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### I16M_P36A_P90I

In some embodiments, a KOD polymerase variant comprises: a M at a position corresponding to I16 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and an I at position P90 of SEQ ID NO: 1. In some embodiments, a KOD polymerase variant comprises: a M at position I16 of SEQ ID NO: 1, an A at position P36 of SEQ ID NO: 1, and an I at position P90 of SEQ ID NO: 1.

### I16M_P36A_P90I_V93I

In some embodiments, a KOD polymerase variant comprises: a M at a position corresponding to I16 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a M at position I16 of SEQ ID NO: 1, an A at position P36 of SEQ ID NO: 1, an I at position P90 of SEQ ID NO: 1, and an I at position V93 of SEQ ID NO: 1.

### I16M_P36A_P90I_V93Q

In some embodiments, a KOD polymerase variant comprises: a M at a position corresponding to I16 of SEQ ID NO: 1, an A at a position corresponding to P36 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, an I at position P90 of SEQ ID NO: 1, and a Q at position V93 of SEQ ID NO: 1. In some embodiments, a KOD polymerase variant comprises: a M at position I16 of SEQ ID NO: 1, an A at position P36 of SEQ ID NO: 1, an I at position P90 of SEQ ID NO: 1, and a Q at position V93 of SEQ ID NO: 1.

### I16M_P36A_P90W

In some embodiments, a KOD polymerase variant comprises: a M at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a W at position P90 of SEQ ID NO: 1. In some embodiments, a KOD polymerase variant comprises: a M at position I16 of SEQ ID NO: 1, an A at position P36 of SEQ ID NO: 1, and a W at position P90 of SEQ ID NO: 1.

### I16M_P36A_P90W_V93I_R97A

In some embodiments, a KOD polymerase variant comprises: a M at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### I16M_P36A_P90Y

In some embodiments, a KOD polymerase variant comprises: a M at a position corresponding to I16 of SEQ ID NO: 1, an A at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a Y at position P90 of SEQ ID NO: 1. In some embodiments, a KOD polymerase variant comprises: a M at position I16 of SEQ ID NO: 1, an A at position P36 of SEQ ID NO: 1, and a Y at position P90 of SEQ ID NO: 1.

### I16M_P36A_P90Y_V93I

In some embodiments, a KOD polymerase variant comprises: a M at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a M at position I16 of SEQ ID NO: 1, an A at position P36 of SEQ ID NO: 1, a Y at position P90 of SEQ ID NO: 1, and an I at position V93 of SEQ ID NO: 1.

### I16M_P36A_P90Y_V93I_R97W

In some embodiments, a KOD polymerase variant comprises: a M at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1.

### I16M_P36A_R97F

In some embodiments, a KOD polymerase variant comprises: a M at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a F at position R97 of SEQ ID NO: 1. In some embodiments, a KOD polymerase variant comprises: a M at position I16 of SEQ ID NO: 1, an A at position P36 of SEQ ID NO: 1, and a F at position R97 of SEQ ID NO: 1.

### I16M_P36A_R97W

In some embodiments, a KOD polymerase variant comprises: a M at a position corresponding to I16 of SEQ ID NO: 1, an A at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a W at position R97 of SEQ ID NO: 1. In some embodiments, a KOD polymerase variant comprises: a M at position I16 of SEQ ID NO: 1, an A at position P36 of SEQ ID NO: 1, and a W at position R97 of SEQ ID NO: 1.

### I16M_P36A_V93I_R97F

In some embodiments, a KOD polymerase variant comprises: a M at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, an A at position P36 of SEQ ID NO: 1, an I at position V93 of SEQ ID NO: 1, and a F at position R97 of SEQ ID NO: 1.

### I16M_P36A_V93K

In some embodiments, a KOD polymerase variant comprises: a M at a position corresponding to I16 of SEQ ID NO: 1, an A at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a K at position V93 of SEQ ID NO: 1. In some embodiments, a KOD polymerase variant comprises: a M at position I16 of SEQ ID NO: 1, an A at position P36 of SEQ ID NO: 1, and a K at position V93 of SEQ ID NO: 1.

### I16M_P36A_V93K_R97W

In some embodiments, a KOD polymerase variant comprises: a M at a position corresponding to I16 of SEQ ID NO: 1, an A at a position corresponding to P36 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a K at position V93 of SEQ ID NO: 1, and a W at position R97 of SEQ ID NO: 1. In some embodiments, a KOD polymerase variant comprises: a M at position I16 of SEQ ID NO: 1, an A at position P36 of SEQ ID NO: 1, a K at position V93 of SEQ ID NO: 1, and a W at position R97 of SEQ ID NO: 1.

### I16M_P36A_V93Q

In some embodiments, a KOD polymerase variant comprises: a M at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a Q at position V93 of SEQ ID NO: 1. In some embodiments, a KOD polymerase variant comprises: a M at position I16 of SEQ ID NO: 1, an A at position P36 of SEQ ID NO: 1, and a Q at position V93 of SEQ ID NO: 1.

### I16M_P36A_V93Q_R97A

In some embodiments, a KOD polymerase variant comprises: a M at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a M at position I16 of SEQ ID NO: 1, an A at position P36 of SEQ ID NO: 1, a Q at position V93 of SEQ ID NO: 1, and an A at position R97 of SEQ ID NO: 1.

### I16M_P36A_V93Q_R97W

In some embodiments, a KOD polymerase variant comprises: a M at a position corresponding to I16 of SEQ ID NO: 1, an A at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a Q at position V93 of SEQ ID NO: 1, and a W at position R97 of SEQ ID NO: 1. In some embodiments, a KOD polymerase variant comprises: a M at position I16 of SEQ ID NO: 1, an A at position P36 of SEQ ID NO: 1, a Q at position V93 of SEQ ID NO: 1, and a W at position R97 of SEQ ID NO: 1.

### I16M_P36A_V93R

In some embodiments, a KOD polymerase variant comprises: a M at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, and a R at position V93 of SEQ ID NO: 1. In some embodiments, a KOD polymerase variant comprises: a M at position I16 of SEQ ID NO: 1, an A at position P36 of SEQ ID NO: 1, and a R at position V93 of SEQ ID NO: 1.

### I16M_P36A_V93R_R97A

In some embodiments, a KOD polymerase variant comprises: a M at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a M at position I16 of SEQ ID NO: 1, an A at position P36 of SEQ ID NO: 1, a R at position V93 of SEQ ID NO: 1, and an A at position R97 of SEQ ID NO: 1.

### I16M_P36A_V93R_R97W

In some embodiments, a KOD polymerase variant comprises: a M at a position corresponding to I16 of SEQ ID NO: 1, an A at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an A at position P36 of SEQ ID NO: 1, a R at position V93 of SEQ ID NO: 1, and a W at position R97 of SEQ ID NO: 1. In some embodiments, a KOD polymerase variant comprises: a M at position I16 of SEQ ID NO: 1, an A at position P36 of SEQ ID NO: 1, a R at position V93 of SEQ ID NO: 1, and a W at position R97 of SEQ ID NO: 1.

### I16M_P36H

In some embodiments, a KOD polymerase variant comprises: a M at a position corresponding to I16 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a H at position P36 of SEQ ID NO: 1. In some embodiments, a KOD polymerase variant comprises: a M at position I16 of SEQ ID NO: 1, and a H at position P36 of SEQ ID NO: 1.

### I16M_P36H_P90F_V93I

In some embodiments, a KOD polymerase variant comprises: a M at a position corresponding to I16 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a M at position I16 of SEQ ID NO: 1, a H at position P36 of SEQ ID NO: 1, a F at position P90 of SEQ ID NO: 1, and an I at position V93 of SEQ ID NO: 1.

### I16M_P36H_R97A

In some embodiments, a KOD polymerase variant comprises: a M at a position corresponding to I16 of SEQ ID NO: 1, a H at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, and an A at position R97 of SEQ ID NO: 1. In some embodiments, a KOD polymerase variant comprises: a M at position I16 of SEQ ID NO: 1, a H at position P36 of SEQ ID NO: 1, and an A at position R97 of SEQ ID NO: 1.

### I16M_P36H_R97F

In some embodiments, a KOD polymerase variant comprises: a M at a position corresponding to I16 of SEQ ID NO: 1, a H at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, and a F at position R97 of SEQ ID NO: 1. In some embodiments, a KOD polymerase variant comprises: a M at position I16 of SEQ ID NO: 1, a H at position P36 of SEQ ID NO: 1, and a F at position R97 of SEQ ID NO: 1.

### I16M_P36H_R97W

In some embodiments, a KOD polymerase variant comprises: a M at a position corresponding to I16 of SEQ ID NO: 1, a H at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, and a W at position R97 of SEQ ID NO: 1. In some embodiments, a KOD polymerase variant comprises: a M at position I16 of SEQ ID NO: 1, a H at position P36 of SEQ ID NO: 1, and a W at position R97 of SEQ ID NO: 1.

### I16M_P36H_V93I

In some embodiments, a KOD polymerase variant comprises: a M at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, and an I at position V93 of SEQ ID NO: 1. In some embodiments, a KOD polymerase variant comprises: a M at position I16 of SEQ ID NO: 1, a H at position P36 of SEQ ID NO: 1, and an I at position V93 of SEQ ID NO: 1.

### I16M_P36H_V93I_R97A

In some embodiments, a KOD polymerase variant comprises: a M at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a H at position P36 of SEQ ID NO: 1, an I at position V93 of SEQ ID NO: 1, and an A at position R97 of SEQ ID NO: 1.

### I16M_P36H_V93I_R97H

In some embodiments, a KOD polymerase variant comprises: a M at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a H at position P36 of SEQ ID NO: 1, an I at position V93 of SEQ ID NO: 1, and a H at position R97 of SEQ ID NO: 1.

### I16M_P36H_V93R

In some embodiments, a KOD polymerase variant comprises: a M at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a H at position P36 of SEQ ID NO: 1, and a R at position V93 of SEQ ID NO: 1. In some embodiments, a KOD polymerase variant comprises: a M at position I16 of SEQ ID NO: 1, a H at position P36 of SEQ ID NO: 1, and a R at position V93 of SEQ ID NO: 1.

### I16M_P36R

In some embodiments, a KOD polymerase variant comprises: a M at a position corresponding to I16 of SEQ ID NO: 1 and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a R at position P36 of SEQ ID NO: 1. In some embodiments, a KOD polymerase variant comprises: a M at position I16 of SEQ ID NO: 1, and a R at position P36 of SEQ ID NO: 1.

### I16M_P36R_R97F

In some embodiments, a KOD polymerase variant comprises: a M at a position corresponding to I16 of SEQ ID NO: 1, a R at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position P36 of SEQ ID NO: 1, and a F at position R97 of SEQ ID NO: 1. In some embodiments, a KOD polymerase variant comprises: a M at position I16 of SEQ ID NO: 1, a R at position P36 of SEQ ID NO: 1, and a F at position R97 of SEQ ID NO: 1.

### I16M_P36R_V93I

In some embodiments, a KOD polymerase variant comprises: a M at a position corresponding to I16 of SEQ ID NO: 1, a R at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position P36 of SEQ ID NO: 1, and an I at position V93 of SEQ ID NO: 1. In some embodiments, a KOD polymerase variant comprises: a M at position I16 of SEQ ID NO: 1, a R at position P36 of SEQ ID NO: 1, and an I at position V93 of SEQ ID NO: 1.

### I16M_P36R_V93Q_R97A

In some embodiments, a KOD polymerase variant comprises: a M at a position corresponding to I16 of SEQ ID NO: 1, a R at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position P36 of SEQ ID NO: 1, a Q at position V93 of SEQ ID NO: 1, and an A at position R97 of SEQ ID NO: 1. In some embodiments, a KOD polymerase variant comprises: a M at position I16 of SEQ ID NO: 1, a R at position P36 of SEQ ID NO: 1, a Q at position V93 of SEQ ID NO: 1, and an A at position R97 of SEQ ID NO: 1.

### I16M_P36S_P90F

In some embodiments, a KOD polymerase variant comprises: a M at a position corresponding to I16 of SEQ ID NO: 1, a S at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P36 of SEQ ID NO: 1, and a F at position P90 of SEQ ID NO: 1. In some embodiments, a KOD polymerase variant comprises: a M at position I16 of SEQ ID NO: 1, a S at position P36 of SEQ ID NO: 1, and a F at position P90 of SEQ ID NO: 1.

### I16M_P36S_P90F_V93I

In some embodiments, a KOD polymerase variant comprises: a M at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a M at position I16 of SEQ ID NO: 1, a S at position P36 of SEQ ID NO: 1, a F at position P90 of SEQ ID NO: 1, and an I at position V93 of SEQ ID NO: 1.

### I16M_P36S_P90Y_V93I

In some embodiments, a KOD polymerase variant comprises: a M at a position corresponding to I16 of SEQ ID NO: 1, a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P36 of SEQ ID NO: 1, a Y at position P90 of SEQ ID NO: 1, and an I at position V93 of SEQ ID NO: 1. In some embodiments, a KOD polymerase variant comprises: a M at position I16 of SEQ ID NO: 1, a S at position P36 of SEQ ID NO: 1, a Y at position P90 of SEQ ID NO: 1, and an I at position V93 of SEQ ID NO: 1.

### I16M_P36S_V93I_R97A

In some embodiments, a KOD polymerase variant comprises: a M at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a S at position P36 of SEQ ID NO: 1, an I at position V93 of SEQ ID NO: 1, and an A at position R97 of SEQ ID NO: 1.

### I16M_P36S_V93Q_R97F

In some embodiments, a KOD polymerase variant comprises: a M at a position corresponding to I16 of SEQ ID NO: 1, a S at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P36 of SEQ ID NO: 1, a Q at position V93 of SEQ ID NO: 1, and a F at position R97 of SEQ ID NO: 1. In some embodiments, a KOD polymerase variant comprises: a M at position I16 of SEQ ID NO: 1, a S at position P36 of SEQ ID NO: 1, a Q at position V93 of SEQ ID NO: 1, and a F at position R97 of SEQ ID NO: 1.

### I16M_P36W

In some embodiments, a KOD polymerase variant comprises: a M at a position corresponding to I16 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a W at position P36 of SEQ ID NO: 1. In some embodiments, a KOD polymerase variant comprises: a M at position I16 of SEQ ID NO: 1, and a W at position P36 of SEQ ID NO: 1.

### I16M_P36W_P90F

In some embodiments, a KOD polymerase variant comprises: a M at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and a F at position P90 of SEQ ID NO: 1. In some embodiments, a KOD polymerase variant comprises: a M at position I16 of SEQ ID NO: 1, a W at position P36 of SEQ ID NO: 1, and a F at position P90 of SEQ ID NO: 1.

### I16M_P36W_P90F_R97H

In some embodiments, a KOD polymerase variant comprises: a M at a position corresponding to I16 of SEQ ID NO: 1, a W at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a F at position P90 of SEQ ID NO: 1, and a H at position R97 of SEQ ID NO: 1. In some embodiments, a KOD polymerase variant comprises: a M at position I16 of SEQ ID NO: 1, a W at position P36 of SEQ ID NO: 1, a F at position P90 of SEQ ID NO: 1, and a H at position R97 of SEQ ID NO: 1.

### I16M_P36W_P90F_V93I

In some embodiments, a KOD polymerase variant comprises: a M at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a M at position I16 of SEQ ID NO: 1, a W at position P36 of SEQ ID NO: 1, a F at position P90 of SEQ ID NO: 1, and an I at position V93 of SEQ ID NO: 1.

### I16M_P36W_P90F_V93I_R97A

In some embodiments, a KOD polymerase variant comprises: a M at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### I16M_P36W_P90F_V93I_R97F

In some embodiments, a KOD polymerase variant comprises: a M at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### I16M_P36W_P90F_V93R_R97A

In some embodiments, a KOD polymerase variant comprises: a M at a position corresponding to I16 of SEQ ID NO: 1, a W at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1.

### I16M_P36W_P90I

In some embodiments, a KOD polymerase variant comprises: a M at a position corresponding to I16 of SEQ ID NO: 1, a W at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and an I at position P90 of SEQ ID NO: 1. In some embodiments, a KOD polymerase variant comprises: a M at position I16 of SEQ ID NO: 1, a W at position P36 of SEQ ID NO: 1, and an I at position P90 of SEQ ID NO: 1.

### I16M_P36W_P90I_V93I

In some embodiments, a KOD polymerase variant comprises: a M at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a M at position I16 of SEQ ID NO: 1, a W at position P36 of SEQ ID NO: 1, an I at position P90 of SEQ ID NO: 1, and an I at position V93 of SEQ ID NO: 1.

### I16M_P36W_P90W

In some embodiments, a KOD polymerase variant comprises: a M at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and a W at position P90 of SEQ ID NO: 1. In some embodiments, a KOD polymerase variant comprises: a M at position I16 of SEQ ID NO: 1, a W at position P36 of SEQ ID NO: 1, and a W at position P90 of SEQ ID NO: 1.

### I16M_P36W_P90W_R97A

In some embodiments, a KOD polymerase variant comprises: a M at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, a W at position P90 of SEQ ID NO: 1, and an A at position R97 of SEQ ID NO: 1.

### I16M_P36W_P90W_V93I

In some embodiments, a KOD polymerase variant comprises: a M at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a M at position I16 of SEQ ID NO: 1, a W at position P36 of SEQ ID NO: 1, a W at position P90 of SEQ ID NO: 1, and an I at position V93 of SEQ ID NO: 1.

### I16M_P36W_P90W_V93I_R97A

In some embodiments, a KOD polymerase variant comprises: a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### I16M_P36W_P90W_V93I_R97F

In some embodiments, a KOD polymerase variant comprises: a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### I16M_P36W_P90W_V93I_R97H

In some embodiments, a KOD polymerase variant comprises: a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1.

### I16M_P36W_P90Y

In some embodiments, a KOD polymerase variant comprises: a M at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and a Y at position P90 of SEQ ID NO: 1. In some embodiments, a KOD polymerase variant comprises: a M at position I16 of SEQ ID NO: 1, a W at position P36 of SEQ ID NO: 1, and a Y at position P90 of SEQ ID NO: 1.

### I16M_P36W_P90Y_V93I

In some embodiments, a KOD polymerase variant comprises: a M at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, a Y at position P90 of SEQ ID NO: 1, and an I at position V93 of SEQ ID NO: 1.

In some embodiments, a KOD polymerase variant comprises: a M at position I16 of SEQ ID NO: 1, a W at position P36 of SEQ ID NO: 1, a Y at position P90 of SEQ ID NO: 1, and an I at position V93 of SEQ ID NO: 1.

### I16M_P36W_R97A

In some embodiments, a KOD polymerase variant comprises: a M at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, and an A at position R97 of SEQ ID NO: 1.

### I16M_P36W_V93I

In some embodiments, a KOD polymerase variant comprises: a M at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and an I at position V93 of SEQ ID NO: 1. In some embodiments, a KOD polymerase variant comprises: a M at position I16 of SEQ ID NO: 1, a W at position P36 of SEQ ID NO: 1, and an I at position V93 of SEQ ID NO: 1.

### I16M_P36W_V93I_R97A

In some embodiments, a KOD polymerase variant comprises: a M at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a W at position P36 of SEQ ID NO: 1, an I at position V93 of SEQ ID NO: 1, and an A at position R97 of SEQ ID NO: 1.

### I16M_P36W_V93I_R97F

In some embodiments, a KOD polymerase variant comprises: a M at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a W at position P36 of SEQ ID NO: 1, an I at position V93 of SEQ ID NO: 1, and a F at position R97 of SEQ ID NO: 1.

### I16M_P36W_V93Q

In some embodiments, a KOD polymerase variant comprises: a M at a position corresponding to I16 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P36 of SEQ ID NO: 1, and a Q at position V93 of SEQ ID NO: 1. In some embodiments, a KOD polymerase variant comprises: a M at position I16 of SEQ ID NO: 1, a W at position P36 of SEQ ID NO: 1, and a Q at position V93 of SEQ ID NO: 1.

### I16M_P90F

In some embodiments, a KOD polymerase variant comprises: a M at a position corresponding to I16 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a F at position P90 of SEQ ID NO: 1. In some embodiments, a KOD polymerase variant comprises: a M at position I16 of SEQ ID NO: 1, and a F at position P90 of SEQ ID NO: 1.

### I16M_P90F_R97A

In some embodiments, a KOD polymerase variant comprises: a M at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and an A at position R97 of SEQ ID NO: 1. In some embodiments, a KOD polymerase variant comprises: a M at position I16 of SEQ ID NO: 1, a F at position P90 of SEQ ID NO: 1, and an A at position R97 of SEQ ID NO: 1.

### I16M_P90F_R97F

In some embodiments, a KOD polymerase variant comprises: a M at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and a F at position R97 of SEQ ID NO: 1. In some embodiments, a KOD polymerase variant comprises: a M at position I16 of SEQ ID NO: 1, a F at position P90 of SEQ ID NO: 1, and a F at position R97 of SEQ ID NO: 1.

### I16M_P90F_R97H

In some embodiments, a KOD polymerase variant comprises: a M at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and a H at position R97 of SEQ ID NO: 1. In some embodiments, a KOD polymerase variant comprises: a M at position I16 of SEQ ID NO: 1, a F at position P90 of SEQ ID NO: 1, and a H at position R97 of SEQ ID NO: 1.

### I16M_P90F_V93I

In some embodiments, a KOD polymerase variant comprises: a M at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and an I at position V93 of SEQ ID NO: 1. In some embodiments, a KOD polymerase variant comprises: a M at position I16 of SEQ ID NO: 1, a F at position P90 of SEQ ID NO: 1, and an I at position V93 of SEQ ID NO: 1.

### I16M_P90F_V93I_R97A

In some embodiments, a KOD polymerase variant comprises: a M at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a F at position P90 of SEQ ID NO: 1, an I at position V93 of SEQ ID NO: 1, and an A at position R97 of SEQ ID NO: 1.

### I16M_P90F_V93I_R97F

In some embodiments, a KOD polymerase variant comprises: a M at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a F at position P90 of SEQ ID NO: 1, an I at position V93 of SEQ ID NO: 1, and a F at position R97 of SEQ ID NO: 1.

### I16M_P90F_V93I_R97H

In some embodiments, a KOD polymerase variant comprises: a M at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a F at position P90 of SEQ ID NO: 1, an I at position V93 of SEQ ID NO: 1, and a H at position R97 of SEQ ID NO: 1.

### I16M_P90F_V93I_R97W

In some embodiments, a KOD polymerase variant comprises: a M at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, a F at position P90 of SEQ ID NO: 1, an I at position V93 of SEQ ID NO: 1, and a W at position R97 of SEQ ID NO: 1.

### I16M_P90F_V93K

In some embodiments, a KOD polymerase variant comprises: a M at a position corresponding to I16 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and a K at position V93 of SEQ ID NO: 1. In some embodiments, a KOD polymerase variant comprises: a M at position I16 of SEQ ID NO: 1, a F at position P90 of SEQ ID NO: 1, and a K at position V93 of SEQ ID NO: 1.

### I16M_P90F_V93Q

In some embodiments, a KOD polymerase variant comprises: a M at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and a Q at position V93 of SEQ ID NO: 1. In some embodiments, a KOD polymerase variant comprises: a M at position I16 of SEQ ID NO: 1, a F at position P90 of SEQ ID NO: 1, and a Q at position V93 of SEQ ID NO: 1.

### I16M_P90F_V93Q_R97A

In some embodiments, a KOD polymerase variant comprises: a M at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a M at position I16 of SEQ ID NO: 1, a F at position P90 of SEQ ID NO: 1, a Q at position V93 of SEQ ID NO: 1, and an A at position R97 of SEQ ID NO: 1.

### I16M_P90F_V93Q_R97H

In some embodiments, a KOD polymerase variant comprises: a M at a position corresponding to I16 of SEQ ID NO: 1, a F at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, a Q at position V93 of SEQ ID NO: 1, and a H at position R97 of SEQ ID NO: 1. In some embodiments, a KOD polymerase variant comprises: a M at position I16 of SEQ ID NO: 1, a F at position P90 of SEQ ID NO: 1, a Q at position V93 of SEQ ID NO: 1, and a H at position R97 of SEQ ID NO: 1.

### I16M_P90F_V93R

In some embodiments, a KOD polymerase variant comprises: a M at a position corresponding to I16 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a F at position P90 of SEQ ID NO: 1, and a R at position V93 of SEQ ID NO: 1. In some embodiments, a KOD polymerase variant comprises: a M at position I16 of SEQ ID NO: 1, a F at position P90 of SEQ ID NO: 1, and a R at position V93 of SEQ ID NO: 1.

### I16M_P90I

In some embodiments, a KOD polymerase variant comprises: a M at a position corresponding to I16 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and an I at position P90 of SEQ ID NO: 1. In some embodiments, a KOD polymerase variant comprises: a M at position I16 of SEQ ID NO: 1, and an I at position P90 of SEQ ID NO: 1.

### I16M_P90I_R97A

In some embodiments, a KOD polymerase variant comprises: a M at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and an A at position R97 of SEQ ID NO: 1. In some embodiments, a KOD polymerase variant comprises: a M at position I16 of SEQ ID NO: 1, an I at position P90 of SEQ ID NO: 1, and an A at position R97 of SEQ ID NO: 1.

### I16M_P90I_R97F

In some embodiments, a KOD polymerase variant comprises: a M at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a F at position R97 of SEQ ID NO: 1. In some embodiments, a KOD polymerase variant comprises: a M at position I16 of SEQ ID NO: 1, an I at position P90 of SEQ ID NO: 1, and a F at position R97 of SEQ ID NO: 1.

### I16M_P90I_R97H

In some embodiments, a KOD polymerase variant comprises: a M at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a H at position R97 of SEQ ID NO: 1. In some embodiments, a KOD polymerase variant comprises: a M at position I16 of SEQ ID NO: 1, an I at position P90 of SEQ ID NO: 1, and a H at position R97 of SEQ ID NO: 1.

### I16M_P90I_R97W

In some embodiments, a KOD polymerase variant comprises: a M at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a W at position R97 of SEQ ID NO: 1. In some embodiments, a KOD polymerase variant comprises: a M at position I16 of SEQ ID NO: 1, an I at position P90 of SEQ ID NO: 1, and a W at position R97 of SEQ ID NO: 1.

### I16M_P90I_V93I

In some embodiments, a KOD polymerase variant comprises: a M at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and an I at position V93 of SEQ ID NO: 1. In some embodiments, a KOD polymerase variant comprises: a M at position I16 of SEQ ID NO: 1, an I at position P90 of SEQ ID NO: 1, and an I at position V93 of SEQ ID NO: 1.

### I16M_P90I_V93I_R97F

In some embodiments, a KOD polymerase variant comprises: a M at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, an I at position P90 of SEQ ID NO: 1, an I at position V93 of SEQ ID NO: 1, and a F at position R97 of SEQ ID NO: 1.

### I16M_P90I_V93I_R97H

In some embodiments, a KOD polymerase variant comprises: a M at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, an I at position P90 of SEQ ID NO: 1, an I at position V93 of SEQ ID NO: 1, and a H at position R97 of SEQ ID NO: 1.

### I16M_P90I_V93I_R97W

In some embodiments, a KOD polymerase variant comprises: a M at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, an I at position P90 of SEQ ID NO: 1, an I at position V93 of SEQ ID NO: 1, and a W at position R97 of SEQ ID NO: 1.

### I16M_P90I_V93K

In some embodiments, a KOD polymerase variant comprises: a M at a position corresponding to I16 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a K at position V93 of SEQ ID NO: 1. In some embodiments, a KOD polymerase variant comprises: a M at position I16 of SEQ ID NO: 1, an I at position P90 of SEQ ID NO: 1, and a K at position V93 of SEQ ID NO: 1.

### I16M_P90I_V93K_R97A

In some embodiments, a KOD polymerase variant comprises: a M at a position corresponding to I16 of SEQ ID NO: 1, an I at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, a K at position V93 of SEQ ID NO: 1, and an A at position R97 of SEQ ID NO: 1. In some embodiments, a KOD polymerase variant comprises: a M at position I16 of SEQ ID NO: 1, an I at position P90 of SEQ ID NO: 1, a K at position V93 of SEQ ID NO: 1, and an A at position R97 of SEQ ID NO: 1.

### I16M_P90I_V93K_R97F

In some embodiments, a KOD polymerase variant comprises: a M at a position corresponding to I16 of SEQ ID NO: 1, an I at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, a K at position V93 of SEQ ID NO: 1, and a F at position R97 of SEQ ID NO: 1. In some embodiments, a KOD polymerase variant comprises: a M at position I16 of SEQ ID NO: 1, an I at position P90 of SEQ ID NO: 1, a K at position V93 of SEQ ID NO: 1, and a F at position R97 of SEQ ID NO: 1.

### I16M_P90I_V93Q

In some embodiments, a KOD polymerase variant comprises: a M at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a Q at position V93 of SEQ ID NO: 1. In some embodiments, a KOD polymerase variant comprises: a M at position I16 of SEQ ID NO: 1, an I at position P90 of SEQ ID NO: 1, and a Q at position V93 of SEQ ID NO: 1.

### I16M_P90I_V93R

In some embodiments, a KOD polymerase variant comprises: a M at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position P90 of SEQ ID NO: 1, and a R at position V93 of SEQ ID NO: 1. In some embodiments, a KOD polymerase variant comprises: a M at position I16 of SEQ ID NO: 1, an I at position P90 of SEQ ID NO: 1, and a R at position V93 of SEQ ID NO: 1.

### I16M_P90S_V93I_R97H

In some embodiments, a KOD polymerase variant comprises: a M at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a S at position P90 of SEQ ID NO: 1, an I at position V93 of SEQ ID NO: 1, and a H at position R97 of SEQ ID NO: 1.

### I16M_P90W

In some embodiments, a KOD polymerase variant comprises: a M at a position corresponding to I16 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a W at position P90 of SEQ ID NO: 1. In some embodiments, a KOD polymerase variant comprises: a M at position I16 of SEQ ID NO: 1, and a W at position P90 of SEQ ID NO: 1.

### I16M_P90W_R97A

In some embodiments, a KOD polymerase variant comprises: a M at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and an A at position R97 of SEQ ID NO: 1. In some embodiments, a KOD polymerase variant comprises: a M at position I16 of SEQ ID NO: 1, a W at position P90 of SEQ ID NO: 1, and an A at position R97 of SEQ ID NO: 1.

### I16M_P90W_R97F

In some embodiments, a KOD polymerase variant comprises: a M at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and a F at position R97 of SEQ ID NO: 1. In some embodiments, a KOD polymerase variant comprises: a M at position I16 of SEQ ID NO: 1, a W at position P90 of SEQ ID NO: 1, and a F at position R97 of SEQ ID NO: 1.

### I16M_P90W_R97H

In some embodiments, a KOD polymerase variant comprises: a M at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and a H at position R97 of SEQ ID NO: 1. In some embodiments, a KOD polymerase variant comprises: a M at position I16 of SEQ ID NO: 1, a W at position P90 of SEQ ID NO: 1, and a H at position R97 of SEQ ID NO: 1.

### I16M_P90W_R97W

In some embodiments, a KOD polymerase variant comprises: a M at a position corresponding to I16 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and a W at position R97 of SEQ ID NO: 1. In some embodiments, a KOD polymerase variant comprises: a M at position I16 of SEQ ID NO: 1, a W at position P90 of SEQ ID NO: 1, and a W at position R97 of SEQ ID NO: 1.

### I16M_P90W_V93I

In some embodiments, a KOD polymerase variant comprises: a M at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and an I at position V93 of SEQ ID NO: 1. In some embodiments, a KOD polymerase variant comprises: a M at position I16 of SEQ ID NO: 1, a W at position P90 of SEQ ID NO: 1, and an I at position V93 of SEQ ID NO: 1.

### I16M_P90W_V93I_R97A

In some embodiments, a KOD polymerase variant comprises: a M at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a W at position P90 of SEQ ID NO: 1, an I at position V93 of SEQ ID NO: 1, and an A at position R97 of SEQ ID NO: 1.

### I16M_P90W_V93I_R97F

In some embodiments, a KOD polymerase variant comprises: a M at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a W at position P90 of SEQ ID NO: 1, an I at position V93 of SEQ ID NO: 1, and a F at position R97 of SEQ ID NO: 1.

### I16M_P90W_V93I_R97H

In some embodiments, a KOD polymerase variant comprises: a M at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a W at position P90 of SEQ ID NO: 1, an I at position V93 of SEQ ID NO: 1, and a H at position R97 of SEQ ID NO: 1.

### I16M_P90W_V93I_R97W

In some embodiments, a KOD polymerase variant comprises: a M at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, a W at position P90 of SEQ ID NO: 1, an I at position V93 of SEQ ID NO: 1, and a W at position R97 of SEQ ID NO: 1.

### I16M_P90W_V93Q

In some embodiments, a KOD polymerase variant comprises: a M at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and a Q at position V93 of SEQ ID NO: 1. In some embodiments, a KOD polymerase variant comprises: a M at position I16 of SEQ ID NO: 1, a W at position P90 of SEQ ID NO: 1, and a Q at position V93 of SEQ ID NO: 1.

### I16M_P90W_V93Q_R97W

In some embodiments, a KOD polymerase variant comprises: a M at a position corresponding to I16 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: a M at position I16 of SEQ ID NO: 1, a W at position P90 of SEQ ID NO: 1, a Q at position V93 of SEQ ID NO: 1, and a W at position R97 of SEQ ID NO: 1.

### I16M_P90W_V93R

In some embodiments, a KOD polymerase variant comprises: a M at a position corresponding to I16 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, and a R at position V93 of SEQ ID NO: 1. In some embodiments, a KOD polymerase variant comprises: a M at position I16 of SEQ ID NO: 1, a W at position P90 of SEQ ID NO: 1, and a R at position V93 of SEQ ID NO: 1.

### I16M_P90W_V93R_R97A

In some embodiments, a KOD polymerase variant comprises: a M at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a M at position I16 of SEQ ID NO: 1, a W at position P90 of SEQ ID NO: 1, a R at position V93 of SEQ ID NO: 1, and an A at position R97 of SEQ ID NO: 1.

### I16M_P90Y

In some embodiments, a KOD polymerase variant comprises: a M at a position corresponding to I16 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a Y at position P90 of SEQ ID NO: 1. In some embodiments, a KOD polymerase variant comprises: a M at position I16 of SEQ ID NO: 1, and a Y at position P90 of SEQ ID NO: 1.

### I16M_P90Y_R97F

In some embodiments, a KOD polymerase variant comprises: a M at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, and a F at position R97 of SEQ ID NO: 1. In some embodiments, a KOD polymerase variant comprises: a M at position I16 of SEQ ID NO: 1, a Y at position P90 of SEQ ID NO: 1, and a F at position R97 of SEQ ID NO: 1.

### I16M_P90Y_R97H

In some embodiments, a KOD polymerase variant comprises: a M at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, and a H at position R97 of SEQ ID NO: 1. In some embodiments, a KOD polymerase variant comprises: a M at position I16 of SEQ ID NO: 1, a Y at position P90 of SEQ ID NO: 1, and a H at position R97 of SEQ ID NO: 1.

### I16M_P90Y_V93I

In some embodiments, a KOD polymerase variant comprises: a M at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, and an I at position V93 of SEQ ID NO: 1. In some embodiments, a KOD polymerase variant comprises: a M at position I16 of SEQ ID NO: 1, a Y at position P90 of SEQ ID NO: 1, and an I at position V93 of SEQ ID NO: 1.

### I16M_P90Y_V93I_R97A

In some embodiments, a KOD polymerase variant comprises: a M at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, a Y at position P90 of SEQ ID NO: 1, an I at position V93 of SEQ ID NO: 1, and an A at position R97 of SEQ ID NO: 1.

### I16M_P90Y_V93I_R97F

In some embodiments, a KOD polymerase variant comprises: a M at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, a Y at position P90 of SEQ ID NO: 1, an I at position V93 of SEQ ID NO: 1, and a F at position R97 of SEQ ID NO: 1.

### I16M_P90Y_V93I_R97H

In some embodiments, a KOD polymerase variant comprises: a M at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, a Y at position P90 of SEQ ID NO: 1, an I at position V93 of SEQ ID NO: 1, and a H at position R97 of SEQ ID NO: 1.

### I16M_P90Y_V93I_R97W

In some embodiments, a KOD polymerase variant comprises: a M at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, a Y at position P90 of SEQ ID NO: 1, an I at position V93 of SEQ ID NO: 1, and a W at position R97 of SEQ ID NO: 1.

### I16M_R97A

In some embodiments, a KOD polymerase variant comprises: a M at a position corresponding to I16 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and an A at position R97 of SEQ ID NO: 1. In some embodiments, a KOD polymerase variant comprises: a M at position I16 of SEQ ID NO: 1, and an A at position R97 of SEQ ID NO: 1.

### I16M_R97F

In some embodiments, a KOD polymerase variant comprises: a M at a position corresponding to I16 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a F at position R97 of SEQ ID NO: 1. In some embodiments, a KOD polymerase variant comprises: a M at position I16 of SEQ ID NO: 1, and a F at position R97 of SEQ ID NO: 1.

### I16M_R97H

In some embodiments, a KOD polymerase variant comprises: a M at a position corresponding to I16 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a H at position R97 of SEQ ID NO: 1. In some embodiments, a KOD polymerase variant comprises: a M at position I16 of SEQ ID NO: 1, and a H at position R97 of SEQ ID NO: 1.

### I16M_V93I

In some embodiments, a KOD polymerase variant comprises: a M at a position corresponding to I16 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and an I at position V93 of SEQ ID NO: 1. In some embodiments, a KOD polymerase variant comprises: a M at position I16 of SEQ ID NO: 1, and an I at position V93 of SEQ ID NO: 1.

### I16M_V93I_R97A

In some embodiments, a KOD polymerase variant comprises: a M at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position V93 of SEQ ID NO: 1, and an A at position R97 of SEQ ID NO: 1. In some embodiments, a KOD polymerase variant comprises: a M at position I16 of SEQ ID NO: 1, an I at position V93 of SEQ ID NO: 1, and an A at position R97 of SEQ ID NO: 1.

### I16M_V93I_R97F

In some embodiments, a KOD polymerase variant comprises: a M at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position V93 of SEQ ID NO: 1, and a F at position R97 of SEQ ID NO: 1. In some embodiments, a KOD polymerase variant comprises: a M at position I16 of SEQ ID NO: 1, an I at position V93 of SEQ ID NO: 1, and a F at position R97 of SEQ ID NO: 1.

### I16M_V93I_R97H

In some embodiments, a KOD polymerase variant comprises: a M at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position V93 of SEQ ID NO: 1, and a H at position R97 of SEQ ID NO: 1. In some embodiments, a KOD polymerase variant comprises: a M at position I16 of SEQ ID NO: 1, an I at position V93 of SEQ ID NO: 1, and a H at position R97 of SEQ ID NO: 1.

### I16M_V93I_R97W

In some embodiments, a KOD polymerase variant comprises: a M at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position V93 of SEQ ID NO: 1, and a W at position R97 of SEQ ID NO: 1. In some embodiments, a KOD polymerase variant comprises: a M at position I16 of SEQ ID NO: 1, an I at position V93 of SEQ ID NO: 1, and a W at position R97 of SEQ ID NO: 1.

### I16M_V93I_R119Q

In some embodiments, a KOD polymerase variant comprises: a M at a position corresponding to I16 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, an I at position V93 of SEQ ID NO: 1, and a Q at position R119 of SEQ ID NO: 1. In some embodiments, a KOD polymerase variant comprises: a M at position I16 of SEQ ID NO: 1, an I at position V93 of SEQ ID NO: 1, and a Q at position R119 of SEQ ID NO: 1.

### I16M_V93K

In some embodiments, a KOD polymerase variant comprises: a M at a position corresponding to I16 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a K at position V93 of SEQ ID NO: 1. In some embodiments, a KOD polymerase variant comprises: a M at position I16 of SEQ ID NO: 1, and a K at position V93 of SEQ ID NO: 1.

### I16M_V93K_R97A

In some embodiments, a KOD polymerase variant comprises: a M at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a K at position V93 of SEQ ID NO: 1, and an A at position R97 of SEQ ID NO: 1. In some embodiments, a KOD polymerase variant comprises: a M at position I16 of SEQ ID NO: 1, a K at position V93 of SEQ ID NO: 1, and an A at position R97 of SEQ ID NO: 1.

### I16M_V93K_R97F

In some embodiments, a KOD polymerase variant comprises: a M at a position corresponding to I16 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a K at position V93 of SEQ ID NO: 1, and a F at position R97 of SEQ ID NO: 1. In some embodiments, a KOD polymerase variant comprises: a M at position I16 of SEQ ID NO: 1, a K at position V93 of SEQ ID NO: 1, and a F at position R97 of SEQ ID NO: 1.

### I16M_V93K_R97W

In some embodiments, a KOD polymerase variant comprises: a M at a position corresponding to I16 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a K at position V93 of SEQ ID NO: 1, and a W at position R97 of SEQ ID NO: 1. In some embodiments, a KOD polymerase variant comprises: a M at position I16 of SEQ ID NO: 1, a K at position V93 of SEQ ID NO: 1, and a W at position R97 of SEQ ID NO: 1.

### I16M_V93Q

In some embodiments, a KOD polymerase variant comprises: a M at a position corresponding to I16 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a Q at position V93 of SEQ ID NO: 1. In some embodiments, a KOD polymerase variant comprises: a M at position I16 of SEQ ID NO: 1, and a Q at position V93 of SEQ ID NO: 1.

### I16M_V93Q_R97A

In some embodiments, a KOD polymerase variant comprises: a M at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Q at position V93 of SEQ ID NO: 1, and an A at position R97 of SEQ ID NO: 1. In some embodiments, a KOD polymerase variant comprises: a M at position I16 of SEQ ID NO: 1, a Q at position V93 of SEQ ID NO: 1, and an A at position R97 of SEQ ID NO: 1.

### I16M_V93Q_R97F

In some embodiments, a KOD polymerase variant comprises: a M at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Q at position V93 of SEQ ID NO: 1, and a F at position R97 of SEQ ID NO: 1. In some embodiments, a KOD polymerase variant comprises: a M at position I16 of SEQ ID NO: 1, a Q at position V93 of SEQ ID NO: 1, and a F at position R97 of SEQ ID NO: 1.

### I16M_V93Q_R97H

In some embodiments, a KOD polymerase variant comprises: a M at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Q at position V93 of SEQ ID NO: 1, and a H at position R97 of SEQ ID NO: 1. In some embodiments, a KOD polymerase variant comprises: a M at position I16 of SEQ ID NO: 1, a Q at position V93 of SEQ ID NO: 1, and a H at position R97 of SEQ ID NO: 1.

### I16M_V93Q_R97W

In some embodiments, a KOD polymerase variant comprises: a M at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a Q at position V93 of SEQ ID NO: 1, and a W at position R97 of SEQ ID NO: 1. In some embodiments, a KOD polymerase variant comprises: a M at position I16 of SEQ ID NO: 1, a Q at position V93 of SEQ ID NO: 1, and a W at position R97 of SEQ ID NO: 1.

### I16M_V93R

In some embodiments, a KOD polymerase variant comprises: a M at a position corresponding to I16 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, and a R at position V93 of SEQ ID NO: 1. In some embodiments, a KOD polymerase variant comprises: a M at position I16 of SEQ ID NO: 1, and a R at position V93 of SEQ ID NO: 1.

### I16M_V93R_R97A

In some embodiments, a KOD polymerase variant comprises: a M at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, and an A at position R97 of SEQ ID NO: 1. In some embodiments, a KOD polymerase variant comprises: a M at position I16 of SEQ ID NO: 1, a R at position V93 of SEQ ID NO: 1, and an A at position R97 of SEQ ID NO: 1.

### I16M_V93R_R97H

In some embodiments, a KOD polymerase variant comprises: a M at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, and a H at position R97 of SEQ ID NO: 1. In some embodiments, a KOD polymerase variant comprises: a M at position I16 of SEQ ID NO: 1, a R at position V93 of SEQ ID NO: 1, and a H at position R97 of SEQ ID NO: 1.

### I16M_V93R_R97H_R119Q

In some embodiments, a KOD polymerase variant comprises: a M at a position corresponding to I16 of SEQ ID NO: 1, a R at a position corresponding to V93 of SEQ ID NO: 1, a H at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, a H at position R97 of SEQ ID NO: 1, and a Q at position R119 of SEQ ID NO: 1. In some embodiments, a KOD polymerase variant comprises: a M at position I16 of SEQ ID NO: 1, a R at position V93 of SEQ ID NO: 1, a H at position R97 of SEQ ID NO: 1, and a Q at position R119 of SEQ ID NO: 1.

### I16M_V93R_R97W

In some embodiments, a KOD polymerase variant comprises: a M at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, and a W at position R97 of SEQ ID NO: 1. In some embodiments, a KOD polymerase variant comprises: a M at position I16 of SEQ ID NO: 1, a R at position V93 of SEQ ID NO: 1, and a W at position R97 of SEQ ID NO: 1.

### I16M_V93R_R119M

In some embodiments, a KOD polymerase variant comprises: a M at a position corresponding to I16 of SEQ ID NO: 1, a R at a position corresponding to V93 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, and a M at position R119 of SEQ ID NO: 1. In some embodiments, a KOD polymerase variant comprises: a M at position I16 of SEQ ID NO: 1, a R at position V93 of SEQ ID NO: 1, and a M at position R119 of SEQ ID NO: 1.

### I16M_V93R_R119Q

In some embodiments, a KOD polymerase variant comprises: a M at a position corresponding to I16 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position I16 of SEQ ID NO: 1, a R at position V93 of SEQ ID NO: 1, and a Q at position R119 of SEQ ID NO: 1. In some embodiments, a KOD polymerase variant comprises: a M at position I16 of SEQ ID NO: 1, a R at position V93 of SEQ ID NO: 1, and a Q at position R119 of SEQ ID NO: 1.

### I16W

In some embodiments, a KOD polymerase variant comprises: a W at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1. In some embodiments, a KOD polymerase variant comprises: a W at position I16 of SEQ ID NO: 1.

### I16W_P36A

In some embodiments, a KOD polymerase variant comprises: a W at a position corresponding to I16 of SEQ ID NO: 1 and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and an A at position P36 of SEQ ID NO: 1. In some embodiments, a KOD polymerase variant comprises: a W at position I16 of SEQ ID NO: 1, and an A at position P36 of SEQ ID NO: 1.

### I16W_P36A_P90F_R97A

In some embodiments, a KOD polymerase variant comprises: a W at a position corresponding to I16 of SEQ ID NO: 1, an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, a F at position P90 of SEQ ID NO: 1, and an A at position R97 of SEQ ID NO: 1. In some embodiments, a KOD polymerase variant comprises: a W at position I16 of SEQ ID NO: 1, an A at position P36 of SEQ ID NO: 1, a F at position P90 of SEQ ID NO: 1, and an A at position R97 of SEQ ID NO: 1.

### I16W_P36A_R97A

In some embodiments, a KOD polymerase variant comprises: a W at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, and an A at position R97 of SEQ ID NO: 1. In some embodiments, a KOD polymerase variant comprises: a W at position I16 of SEQ ID NO: 1, an A at position P36 of SEQ ID NO: 1, and an A at position R97 of SEQ ID NO: 1.

### I16W_P36A_R97F

In some embodiments, a KOD polymerase variant comprises: a W at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, and a F at position R97 of SEQ ID NO: 1. In some embodiments, a KOD polymerase variant comprises: a W at position I16 of SEQ ID NO: 1, an A at position P36 of SEQ ID NO: 1, and a F at position R97 of SEQ ID NO: 1.

### I16W_P36A_R97H

In some embodiments, a KOD polymerase variant comprises: a W at a position corresponding to I16 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, and a H at position R97 of SEQ ID NO: 1. In some embodiments, a KOD polymerase variant comprises: a W at position I16 of SEQ ID NO: 1, an A at position P36 of SEQ ID NO: 1, and a H at position R97 of SEQ ID NO: 1.

### I16W_P36A_V93I

In some embodiments, a KOD polymerase variant comprises: a W at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, and an I at position V93 of SEQ ID NO: 1. In some embodiments, a KOD polymerase variant comprises: a W at position I16 of SEQ ID NO: 1, an A at position P36 of SEQ ID NO: 1, and an I at position V93 of SEQ ID NO: 1.

### I16W_P36A_V93I_R97A

In some embodiments, a KOD polymerase variant comprises: a W at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, an A at position P36 of SEQ ID NO: 1, an I at position V93 of SEQ ID NO: 1, and an A at position R97 of SEQ ID NO: 1.

### I16W_P36A_V93I_R97F

In some embodiments, a KOD polymerase variant comprises: a W at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a W at position I16 of SEQ ID NO: 1, an A at position P36 of SEQ ID NO: 1, an I at position V93 of SEQ ID NO: 1, and a F at position R97 of SEQ ID NO: 1.

### I16W_P36A_V93I_R97H

In some embodiments, a KOD polymerase variant comprises: a W at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, an A at position P36 of SEQ ID NO: 1, an I at position V93 of SEQ ID NO: 1, and a H at position R97 of SEQ ID NO: 1.

### I16W_P36A_V93I_R97W

In some embodiments, a KOD polymerase variant comprises: a W at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a W at position I16 of SEQ ID NO: 1, an A at position P36 of SEQ ID NO: 1, an I at position V93 of SEQ ID NO: 1, and a W at position R97 of SEQ ID NO: 1.

### I16W_P36A_V93K_R97H

In some embodiments, a KOD polymerase variant comprises: a W at a position corresponding to I16 of SEQ ID NO: 1, an A at a position corresponding to P36 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an A at position P36 of SEQ ID NO: 1, a K at position V93 of SEQ ID NO: 1, and a H at position R97 of SEQ ID NO: 1. In some embodiments, a KOD polymerase variant comprises: a W at position I16 of SEQ ID NO: 1, an A at position P36 of SEQ ID NO: 1, a K at position V93 of SEQ ID NO: 1, and a H at position R97 of SEQ ID NO: 1.

### I16W_P36H

In some embodiments, a KOD polymerase variant comprises: a W at a position corresponding to I16 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a H at position P36 of SEQ ID NO: 1. In some embodiments, a KOD polymerase variant comprises: a W at position I16 of SEQ ID NO: 1, and a H at position P36 of SEQ ID NO: 1.

### I16W_P36H_R97A

In some embodiments, a KOD polymerase variant comprises: a W at a position corresponding to I16 of SEQ ID NO: 1, a H at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, and an A at position R97 of SEQ ID NO: 1. In some embodiments, a KOD polymerase variant comprises: a W at position I16 of SEQ ID NO: 1, a H at position P36 of SEQ ID NO: 1, and an A at position R97 of SEQ ID NO: 1.

### I16W_P36H_V93I

In some embodiments, a KOD polymerase variant comprises: a W at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, and an I at position V93 of SEQ ID NO: 1. In some embodiments, a KOD polymerase variant comprises: a W at position I16 of SEQ ID NO: 1, a H at position P36 of SEQ ID NO: 1, and an I at position V93 of SEQ ID NO: 1.

### I16W_P36H_V93I_R97A

In some embodiments, a KOD polymerase variant comprises: a W at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, a H at position P36 of SEQ ID NO: 1, an I at position V93 of SEQ ID NO: 1, and an A at position R97 of SEQ ID NO: 1.

### I16W_P36H_V93I_R97F

In some embodiments, a KOD polymerase variant comprises: a W at a position corresponding to I16 of SEQ ID NO: 1, a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, an I at position V93 of SEQ ID NO: 1, and a F at position R97 of SEQ ID NO: 1. In some embodiments, a KOD polymerase variant comprises: a W at position I16 of SEQ ID NO: 1, a H at position P36 of SEQ ID NO: 1, an I at position V93 of SEQ ID NO: 1, and a F at position R97 of SEQ ID NO: 1.

### I16W_P36H_V93I_R97H

In some embodiments, a KOD polymerase variant comprises: a W at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, a H at position P36 of SEQ ID NO: 1, an I at position V93 of SEQ ID NO: 1, and a H at position R97 of SEQ ID NO:1.

### I16W_P36S

In some embodiments, a KOD polymerase variant comprises: a W at a position corresponding to I16 of SEQ ID NO: 1 and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a S at position P36 of SEQ ID NO: 1. In some embodiments, a KOD polymerase variant comprises: a W at position I16 of SEQ ID NO: 1, and a S at position P36 of SEQ ID NO: 1.

### I16W_P36S_P90W_V93I_R97A

In some embodiments, a KOD polymerase variant comprises: a W at a position corresponding to I16 of SEQ ID NO: 1, a S at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1.

### I16W_P36S_R97A

In some embodiments, a KOD polymerase variant comprises: a W at a position corresponding to I16 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, and an A at position R97 of SEQ ID NO: 1. In some embodiments, a KOD polymerase variant comprises: a W at position I16 of SEQ ID NO: 1, a S at position P36 of SEQ ID NO: 1, and an A at position R97 of SEQ ID NO: 1.

### I16W_P36S_V93I

In some embodiments, a KOD polymerase variant comprises: a W at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, and an I at position V93 of SEQ ID NO: 1. In some embodiments, a KOD polymerase variant comprises: a W at position I16 of SEQ ID NO: 1, a S at position P36 of SEQ ID NO: 1, and an I at position V93 of SEQ ID NO: 1.

### I16W_P36S_V93I_R97A

In some embodiments, a KOD polymerase variant comprises: a W at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, a S at position P36 of SEQ ID NO: 1, an I at position V93 of SEQ ID NO: 1, and an A at position R97 of SEQ ID NO: 1.

### I16W_P36S_V93I_R97H

In some embodiments, a KOD polymerase variant comprises: a W at a position corresponding to I16 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, a S at position P36 of SEQ ID NO: 1, an I at position V93 of SEQ ID NO: 1, and a H at position R97 of SEQ ID NO: 1.

### I16W_P36S_V93R_R97A

In some embodiments, a KOD polymerase variant comprises: a W at a position corresponding to I16 of SEQ ID NO: 1, a S at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a S at position P36 of SEQ ID NO: 1, a R at position V93 of SEQ ID NO: 1, and an A at position R97 of SEQ ID NO: 1. In some embodiments, a KOD polymerase variant comprises: a W at position I16 of SEQ ID NO: 1, a S at position P36 of SEQ ID NO: 1, a R at position V93 of SEQ ID NO: 1, and an A at position R97 of SEQ ID NO: 1.

### I16W_P90F

In some embodiments, a KOD polymerase variant comprises: a W at a position corresponding to I16 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a F at position P90 of SEQ ID NO: 1. In some embodiments, a KOD polymerase variant comprises: a W at position I16 of SEQ ID NO: 1, and a F at position P90 of SEQ ID NO: 1.

### I16W_P90F_R97A

In some embodiments, a KOD polymerase variant comprises: a W at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, and an A at position R97 of SEQ ID NO: 1. In some embodiments, a KOD polymerase variant comprises: a W at position I16 of SEQ ID NO: 1, a F at position P90 of SEQ ID NO: 1, and an A at position R97 of SEQ ID NO: 1.

### I16W_P90F_V93I

In some embodiments, a KOD polymerase variant comprises: a W at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, and an I at position V93 of SEQ ID NO: 1. In some embodiments, a KOD polymerase variant comprises: a W at position I16 of SEQ ID NO: 1, a F at position P90 of SEQ ID NO: 1, and an I at position V93 of SEQ ID NO: 1.

### I16W_P90F_V93I_R97A

In some embodiments, a KOD polymerase variant comprises: a W at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, a F at position P90 of SEQ ID NO: 1, an I at position V93 of SEQ ID NO: 1, and an A at position R97 of SEQ ID NO: 1.

### I16W_P90F_V93I_R97F

In some embodiments, a KOD polymerase variant comprises: a W at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a W at position I16 of SEQ ID NO: 1, a F at position P90 of SEQ ID NO: 1, an I at position V93 of SEQ ID NO: 1, and a F at position R97 of SEQ ID NO: 1.

### I16W_P90F_V93I_R97H

In some embodiments, a KOD polymerase variant comprises: a W at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, a F at position P90 of SEQ ID NO: 1, an I at position V93 of SEQ ID NO: 1, and a H at position R97 of SEQ ID NO: 1.

### I16W_P90F_V93I_R97W

In some embodiments, a KOD polymerase variant comprises: a W at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a W at position I16 of SEQ ID NO: 1, a F at position P90 of SEQ ID NO: 1, an I at position V93 of SEQ ID NO: 1, and a W at position R97 of SEQ ID NO: 1.

### I16W_P90F_V93R_R97F

In some embodiments, a KOD polymerase variant comprises: a W at a position corresponding to I16 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: a W at position I16 of SEQ ID NO: 1, a F at position P90 of SEQ ID NO: 1, a R at position V93 of SEQ ID NO: 1, and a F at position R97 of SEQ ID NO: 1.

### I16W_P90I

In some embodiments, a KOD polymerase variant comprises: a W at a position corresponding to I16 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and an I at position P90 of SEQ ID NO: 1. In some embodiments, a KOD polymerase variant comprises: a W at position I16 of SEQ ID NO: 1, and an I at position P90 of SEQ ID NO: 1.

### I16W_P90I_R97A

In some embodiments, a KOD polymerase variant comprises: a W at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position P90 of SEQ ID NO: 1, and an A at position R97 of SEQ ID NO: 1. In some embodiments, a KOD polymerase variant comprises: a W at position I16 of SEQ ID NO: 1, an I at position P90 of SEQ ID NO: 1, and an A at position R97 of SEQ ID NO: 1.

### I16W_P90I_V93I

In some embodiments, a KOD polymerase variant comprises: a W at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position P90 of SEQ ID NO: 1, and an I at position V93 of SEQ ID NO: 1. In some embodiments, a KOD polymerase variant comprises: a W at position I16 of SEQ ID NO: 1, an I at position P90 of SEQ ID NO: 1, and an I at position V93 of SEQ ID NO: 1.

### I16W_P90I_V93I_R97A

In some embodiments, a KOD polymerase variant comprises: a W at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, an I at position P90 of SEQ ID NO: 1, an I at position V93 of SEQ ID NO: 1, and an A at position R97 of SEQ ID NO: 1.

### I16W_P90I_V93I_R97F

In some embodiments, a KOD polymerase variant comprises: a W at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a W at position I16 of SEQ ID NO: 1, an I at position P90 of SEQ ID NO: 1, an I at position V93 of SEQ ID NO: 1, and a F at position R97 of SEQ ID NO: 1.

### I16W_P90I_V93I_R97H

In some embodiments, a KOD polymerase variant comprises: a W at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, an I at position P90 of SEQ ID NO: 1, an I at position V93 of SEQ ID NO: 1, and a H at position R97 of SEQ ID NO: 1.

### I16W_P90W

In some embodiments, a KOD polymerase variant comprises: a W at a position corresponding to I16 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a W at position P90 of SEQ ID NO: 1. In some embodiments, a KOD polymerase variant comprises: a W at position I16 of SEQ ID NO: 1, and a W at position P90 of SEQ ID NO: 1.

### I16W_P90W_R97F

In some embodiments, a KOD polymerase variant comprises: a W at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a W at position P90 of SEQ ID NO: 1, and a F at position R97 of SEQ ID NO: 1. In some embodiments, a KOD polymerase variant comprises: a W at position I16 of SEQ ID NO: 1, a W at position P90 of SEQ ID NO: 1, and a F at position R97 of SEQ ID NO: 1.

### I16W_P90W_V93I

In some embodiments, a KOD polymerase variant comprises: a W at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a W at position P90 of SEQ ID NO: 1, and an I at position V93 of SEQ ID NO: 1. In some embodiments, a KOD polymerase variant comprises: a W at position I16 of SEQ ID NO: 1, a W at position P90 of SEQ ID NO: 1, and an I at position V93 of SEQ ID NO: 1.

### I16W_P90Y

In some embodiments, a KOD polymerase variant comprises: a W at a position corresponding to I16 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a Y at position P90 of SEQ ID NO: 1. In some embodiments, a KOD polymerase variant comprises: a W at position I16 of SEQ ID NO: 1, and a Y at position P90 of SEQ ID NO: 1.

### I16W_P90Y_V93I

In some embodiments, a KOD polymerase variant comprises: a W at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Y at position P90 of SEQ ID NO: 1, and an I at position V93 of SEQ ID NO: 1. In some embodiments, a KOD polymerase variant comprises: a W at position I16 of SEQ ID NO: 1, a Y at position P90 of SEQ ID NO: 1, and an I at position V93 of SEQ ID NO: 1.

### I16W_P90Y_V93I_R97A

In some embodiments, a KOD polymerase variant comprises: a W at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, a Y at position P90 of SEQ ID NO: 1, an I at position V93 of SEQ ID NO: 1, and an A at position R97 of SEQ ID NO: 1.

### I16W_P90Y_V93I_R97H

In some embodiments, a KOD polymerase variant comprises: a W at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, a Y at position P90 of SEQ ID NO: 1, an I at position V93 of SEQ ID NO: 1, and a H at position R97 of SEQ ID NO: 1.

### I16W_P90Y_V93K_R97A

In some embodiments, a KOD polymerase variant comprises: a W at a position corresponding to I16 of SEQ ID NO: 1, a Y at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Y at position P90 of SEQ ID NO: 1, a K at position V93 of SEQ ID NO: 1, and an A at position R97 of SEQ ID NO: 1. In some embodiments, a KOD polymerase variant comprises: a W at position I16 of SEQ ID NO: 1, a Y at position P90 of SEQ ID NO: 1, a K at position V93 of SEQ ID NO: 1, and an A at position R97 of SEQ ID NO: 1.

### I16W_P90Y_V93Q_R97H

In some embodiments, a KOD polymerase variant comprises: a W at a position corresponding to I16 of SEQ ID NO: 1, a Y at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Y at position P90 of SEQ ID NO: 1, a Q at position V93 of SEQ ID NO: 1, and a H at position R97 of SEQ ID NO: 1. In some embodiments, a KOD polymerase variant comprises: a W at position I16 of SEQ ID NO: 1, a Y at position P90 of SEQ ID NO: 1, a Q at position V93 of SEQ ID NO: 1, and a H at position R97 of SEQ ID NO: 1.

### I16W_R97A

In some embodiments, a KOD polymerase variant comprises: a W at a position corresponding to I16 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and an A at position R97 of SEQ ID NO: 1. In some embodiments, a KOD polymerase variant comprises: a W at position I16 of SEQ ID NO: 1, and an A at position R97 of SEQ ID NO: 1.

### I16W_R97F

In some embodiments, a KOD polymerase variant comprises: a W at a position corresponding to I16 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a F at position R97 of SEQ ID NO: 1. In some embodiments, a KOD polymerase variant comprises: a W at position I16 of SEQ ID NO: 1, and a F at position R97 of SEQ ID NO: 1.

### I16W_R97H

In some embodiments, a KOD polymerase variant comprises: a W at a position corresponding to I16 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a H at position R97 of SEQ ID NO: 1. In some embodiments, a KOD polymerase variant comprises: a W at position I16 of SEQ ID NO: 1, and a H at position R97 of SEQ ID NO: 1.

### I16W_R97W

In some embodiments, a KOD polymerase variant comprises: a W at a position corresponding to I16 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a W at position R97 of SEQ ID NO: 1. In some embodiments, a KOD polymerase variant comprises: a W at position I16 of SEQ ID NO: 1, and a W at position R97 of SEQ ID NO: 1.

### I16W_V93I

In some embodiments, a KOD polymerase variant comprises: a W at a position corresponding to I16 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and an I at position V93 of SEQ ID NO: 1. In some embodiments, a KOD polymerase variant comprises: a W at position I16 of SEQ ID NO: 1, and an I at position V93 of SEQ ID NO: 1.

### I16W_V93I_R97A

In some embodiments, a KOD polymerase variant comprises: a W at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position V93 of SEQ ID NO: 1, and an A at position R97 of SEQ ID NO: 1. In some embodiments, a KOD polymerase variant comprises: a W at position I16 of SEQ ID NO: 1, an I at position V93 of SEQ ID NO: 1, and an A at position R97 of SEQ ID NO: 1.

### I16W_V93I_R97F

In some embodiments, a KOD polymerase variant comprises: a W at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position V93 of SEQ ID NO: 1, and a F at position R97 of SEQ ID NO: 1. In some embodiments, a KOD polymerase variant comprises: a W at position I16 of SEQ ID NO: 1, an I at position V93 of SEQ ID NO: 1, and a F at position R97 of SEQ ID NO: 1.

### I16W_V93I_R97H

In some embodiments, a KOD polymerase variant comprises: a W at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position V93 of SEQ ID NO: 1, and a H at position R97 of SEQ ID NO: 1. In some embodiments, a KOD polymerase variant comprises: a W at position I16 of SEQ ID NO: 1, an I at position V93 of SEQ ID NO: 1, and a H at position R97 of SEQ ID NO: 1.

### I16W_V93I_R97W

In some embodiments, a KOD polymerase variant comprises: a W at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, an I at position V93 of SEQ ID NO: 1, and a W at position R97 of SEQ ID NO: 1. In some embodiments, a KOD polymerase variant comprises: a W at position I16 of SEQ ID NO: 1, an I at position V93 of SEQ ID NO: 1, and a W at position R97 of SEQ ID NO: 1.

### I16W_V93Q

In some embodiments, a KOD polymerase variant comprises: a W at a position corresponding to I16 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a Q at position V93 of SEQ ID NO: 1. In some embodiments, a KOD polymerase variant comprises: a W at position I16 of SEQ ID NO: 1, and a Q at position V93 of SEQ ID NO: 1.

### I16W_V93Q_R97A

In some embodiments, a KOD polymerase variant comprises: a W at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Q at position V93 of SEQ ID NO: 1, and an A at position R97 of SEQ ID NO: 1. In some embodiments, a KOD polymerase variant comprises: a W at position I16 of SEQ ID NO: 1, a Q at position V93 of SEQ ID NO: 1, and an A at position R97 of SEQ ID NO: 1.

### I16W_V93Q_R97W

In some embodiments, a KOD polymerase variant comprises: a W at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a Q at position V93 of SEQ ID NO: 1, and a W at position R97 of SEQ ID NO: 1. In some embodiments, a KOD polymerase variant comprises: a W at position I16 of SEQ ID NO: 1, a Q at position V93 of SEQ ID NO: 1, and a W at position R97 of SEQ ID NO: 1.

### I16W_V93R

In some embodiments, a KOD polymerase variant comprises: a W at a position corresponding to I16 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a R at position V93 of SEQ ID NO: 1. In some embodiments, a KOD polymerase variant comprises: a W at position I16 of SEQ ID NO: 1, and a R at position V93 of SEQ ID NO: 1.

### I16W_V93R_R97A

In some embodiments, a KOD polymerase variant comprises: a W at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a R at position V93 of SEQ ID NO: 1, and an A at position R97 of SEQ ID NO: 1. In some embodiments, a KOD polymerase variant comprises: a W at position I16 of SEQ ID NO: 1, a R at position V93 of SEQ ID NO: 1, and an A at position R97 of SEQ ID NO: 1.

### I16W_V93R_R97H

In some embodiments, a KOD polymerase variant comprises: a W at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a R at position V93 of SEQ ID NO: 1, and a H at position R97 of SEQ ID NO: 1. In some embodiments, a KOD polymerase variant comprises: a W at position I16 of SEQ ID NO: 1, a R at position V93 of SEQ ID NO: 1, and a H at position R97 of SEQ ID NO: 1.

### I16W_V93R_R97W

In some embodiments, a KOD polymerase variant comprises: a W at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a R at position V93 of SEQ ID NO: 1, and a W at position R97 of SEQ ID NO: 1. In some embodiments, a KOD polymerase variant comprises: a W at position I16 of SEQ ID NO: 1, a R at position V93 of SEQ ID NO: 1, and a W at position R97 of SEQ ID NO: 1.

### P36A

In some embodiments, a KOD polymerase variant comprises: an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1. In some embodiments, a KOD polymerase variant comprises: an A at position P36 of SEQ ID NO: 1.

### P36A_P90F

In some embodiments, a KOD polymerase variant comprises: an A at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a F at position P90 of SEQ ID NO: 1. In some embodiments, a KOD polymerase variant comprises: an A at position P36 of SEQ ID NO: 1, and a F at position P90 of SEQ ID NO: 1.

### P36A_P90F_R97A

In some embodiments, a KOD polymerase variant comprises: an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and an A at position R97 of SEQ ID NO: 1. In some embodiments, a KOD polymerase variant comprises: an A at position P36 of SEQ ID NO: 1, a F at position P90 of SEQ ID NO: 1, and an A at position R97 of SEQ ID NO: 1.

### P36A_P90F_R97F

In some embodiments, a KOD polymerase variant comprises: an A at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and a F at position R97 of SEQ ID NO: 1. In some embodiments, a KOD polymerase variant comprises: an A at position P36 of SEQ ID NO: 1, a F at position P90 of SEQ ID NO: 1, and a F at position R97 of SEQ ID NO: 1.

### P36A_P90F_V93I

In some embodiments, a KOD polymerase variant comprises: an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and an I at position V93 of SEQ ID NO: 1. In some embodiments, a KOD polymerase variant comprises: an A at position P36 of SEQ ID NO: 1, a F at position P90 of SEQ ID NO: 1, and an I at position V93 of SEQ ID NO: 1.

### P36A_P90F_V93I_R97A

In some embodiments, a KOD polymerase variant comprises: an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, a F at position P90 of SEQ ID NO: 1, an I at position V93 of SEQ ID NO: 1, and an A at position R97 of SEQ ID NO: 1.

### P36A_P90F_V93I_R97F

In some embodiments, a KOD polymerase variant comprises: an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, a F at position P90 of SEQ ID NO: 1, an I at position V93 of SEQ ID NO: 1, and a F at position R97 of SEQ ID NO: 1.

### P36A_P90F_V93I_R97H

In some embodiments, a KOD polymerase variant comprises: an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: an A at position P36 of SEQ ID NO: 1, a F at position P90 of SEQ ID NO: 1, an I at position V93 of SEQ ID NO: 1, and a H at position R97 of SEQ ID NO: 1.

### P36A_P90F_V93I_R97W

In some embodiments, a KOD polymerase variant comprises: an A at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: an A at position P36 of SEQ ID NO: 1, a F at position P90 of SEQ ID NO: 1, an I at position V93 of SEQ ID NO: 1, and a W at position R97 of SEQ ID NO: 1.

### P36A_P90F_V93K

In some embodiments, a KOD polymerase variant comprises: an A at a position corresponding to P36 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and a K at position V93 of SEQ ID NO: 1. In some embodiments, a KOD polymerase variant comprises: an A at position P36 of SEQ ID NO: 1, a F at position P90 of SEQ ID NO: 1, and a K at position V93 of SEQ ID NO: 1.

### P36A_P90F_V93Q

In some embodiments, a KOD polymerase variant comprises: an A at a position corresponding to P36 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and a Q at position V93 of SEQ ID NO: 1. In some embodiments, a KOD polymerase variant comprises: an A at position P36 of SEQ ID NO: 1, a F at position P90 of SEQ ID NO: 1, and a Q at position V93 of SEQ ID NO: 1.

### P36A_P90F_V93R

In some embodiments, a KOD polymerase variant comprises: an A at a position corresponding to P36 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, and a R at position V93 of SEQ ID NO: 1. In some embodiments, a KOD polymerase variant comprises: an A at position P36 of SEQ ID NO: 1, a F at position P90 of SEQ ID NO: 1, and a R at position V93 of SEQ ID NO: 1.

### P36A_P90F_V93R_R97A

In some embodiments, a KOD polymerase variant comprises: an A at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: an A at position P36 of SEQ ID NO: 1, a F at position P90 of SEQ ID NO: 1, a R at position V93 of SEQ ID NO: 1, and an A at position R97 of SEQ ID NO: 1.

### P36A_P90F_V93R_R97F

In some embodiments, a KOD polymerase variant comprises: an A at a position corresponding to P36 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: an A at position P36 of SEQ ID NO: 1, a F at position P90 of SEQ ID NO: 1, a R at position V93 of SEQ ID NO: 1, and a F at position R97 of SEQ ID NO: 1.

### P36A_P90I

In some embodiments, a KOD polymerase variant comprises: an A at a position corresponding to P36 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and an I at position P90 of SEQ ID NO: 1. In some embodiments, a KOD polymerase variant comprises: an A at position P36 of SEQ ID NO: 1, and an I at position P90 of SEQ ID NO: 1.

### P36A_P90I_R97A

In some embodiments, a KOD polymerase variant comprises: an A at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and an A at position R97 of SEQ ID NO: 1. In some embodiments, a KOD polymerase variant comprises: an A at position P36 of SEQ ID NO: 1, an I at position P90 of SEQ ID NO: 1, and an A at position R97 of SEQ ID NO: 1.

### P36A_P90I_R97F

In some embodiments, a KOD polymerase variant comprises: an A at a position corresponding to P36 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and a F at position R97 of SEQ ID NO: 1. In some embodiments, a KOD polymerase variant comprises: an A at position P36 of SEQ ID NO: 1, an I at position P90 of SEQ ID NO: 1, and a F at position R97 of SEQ ID NO: 1.

### P36A_P90I_V93I

In some embodiments, a KOD polymerase variant comprises: an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and an I at position V93 of SEQ ID NO: 1. In some embodiments, a KOD polymerase variant comprises: an A at position P36 of SEQ ID NO: 1, an I at position P90 of SEQ ID NO: 1, and an I at position V93 of SEQ ID NO: 1.

### P36A_P90I_V93I_R97A

In some embodiments, a KOD polymerase variant comprises: an A at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, an I at position P90 of SEQ ID NO: 1, an I at position V93 of SEQ ID NO: 1, and an A at position R97 of SEQ ID NO: 1.

### P36A_P90I_V93I_R97F

In some embodiments, a KOD polymerase variant comprises: an A at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, an I at position P90 of SEQ ID NO: 1, an I at position V93 of SEQ ID NO: 1, and a F at position R97 of SEQ ID NO: 1.

### P36A_P90I_V93I_R97H

In some embodiments, a KOD polymerase variant comprises: an A at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: an A at position P36 of SEQ ID NO: 1, an I at position P90 of SEQ ID NO: 1, an I at position V93 of SEQ ID NO: 1, and a H at position R97 of SEQ ID NO: 1.

### P36A_P90I_V93I_R97W

In some embodiments, a KOD polymerase variant comprises: an A at a position corresponding to P36 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: an A at position P36 of SEQ ID NO: 1, an I at position P90 of SEQ ID NO: 1, an I at position V93 of SEQ ID NO: 1, and a W at position R97 of SEQ ID NO: 1.

### P36A_P90I_V93K

In some embodiments, a KOD polymerase variant comprises: an A at a position corresponding to P36 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and a K at position V93 of SEQ ID NO: 1. In some embodiments, a KOD polymerase variant comprises: an A at position P36 of SEQ ID NO: 1, an I at position P90 of SEQ ID NO: 1, and a K at position V93 of SEQ ID NO: 1.

### P36A_P90I_V93K_R97F

In some embodiments, a KOD polymerase variant comprises: an A at a position corresponding to P36 of SEQ ID NO: 1, an I at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, a K at position V93 of SEQ ID NO: 1, and a F at position R97 of SEQ ID NO: 1. In some embodiments, a KOD polymerase variant comprises: an A at position P36 of SEQ ID NO: 1, an I at position P90 of SEQ ID NO: 1, a K at position V93 of SEQ ID NO: 1, and a F at position R97 of SEQ ID NO: 1.

### P36A_P90I_V93Q

In some embodiments, a KOD polymerase variant comprises: an A at a position corresponding to P36 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and a Q at position V93 of SEQ ID NO: 1. In some embodiments, a KOD polymerase variant comprises: an A at position P36 of SEQ ID NO: 1, an I at position P90 of SEQ ID NO: 1, and a Q at position V93 of SEQ ID NO: 1.

### P36A_P90I_V93R

In some embodiments, a KOD polymerase variant comprises: an A at a position corresponding to P36 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, and a R at position V93 of SEQ ID NO: 1. In some embodiments, a KOD polymerase variant comprises: an A at position P36 of SEQ ID NO: 1, an I at position P90 of SEQ ID NO: 1, and a R at position V93 of SEQ ID NO: 1.

### P36A_P90I_V93R_R97A

In some embodiments, a KOD polymerase variant comprises: an A at a position corresponding to P36 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: an A at position P36 of SEQ ID NO: 1, an I at position P90 of SEQ ID NO: 1, a R at position V93 of SEQ ID NO: 1, and an A at position R97 of SEQ ID NO: 1.

### P36A_P90I_V93R_R97H

In some embodiments, a KOD polymerase variant comprises: an A at a position corresponding to P36 of SEQ ID NO: 1, an I at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position P90 of SEQ ID NO: 1, a R at position V93 of SEQ ID NO: 1, and a H at position R97 of SEQ ID NO: 1. In some embodiments, a KOD polymerase variant comprises: an A at position P36 of SEQ ID NO: 1, an I at position P90 of SEQ ID NO: 1, a R at position V93 of SEQ ID NO: 1, and a H at position R97 of SEQ ID NO: 1.

### P36A_P90S_R97A

In some embodiments, a KOD polymerase variant comprises: an A at a position corresponding to P36 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, and an A at position R97 of SEQ ID NO: 1. In some embodiments, a KOD polymerase variant comprises: an A at position P36 of SEQ ID NO: 1, a S at position P90 of SEQ ID NO: 1, and an A at position R97 of SEQ ID NO: 1.

### P36A_P90S_R97F

In some embodiments, a KOD polymerase variant comprises: an A at a position corresponding to P36 of SEQ ID NO: 1, a S at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, and a F at position R97 of SEQ ID NO: 1. In some embodiments, a KOD polymerase variant comprises: an A at position P36 of SEQ ID NO: 1, a S at position P90 of SEQ ID NO: 1, and a F at position R97 of SEQ ID NO: 1.

### P36A_P90S_V93I_R97A

In some embodiments, a KOD polymerase variant comprises: an A at a position corresponding to P36 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, a S at position P90 of SEQ ID NO: 1, an I at position V93 of SEQ ID NO: 1, and an A at position R97 of SEQ ID NO: 1.

### P36A_P90S_V93I_R97F

In some embodiments, a KOD polymerase variant comprises: an A at a position corresponding to P36 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, a S at position P90 of SEQ ID NO: 1, an I at position V93 of SEQ ID NO: 1, and a F at position R97 of SEQ ID NO: 1.

### P36A_P90S_V93I_R97H

In some embodiments, a KOD polymerase variant comprises: an A at a position corresponding to P36 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: an A at position P36 of SEQ ID NO: 1, a S at position P90 of SEQ ID NO: 1, an I at position V93 of SEQ ID NO: 1, and a H at position R97 of SEQ ID NO: 1.

### P36A_P90S_V93R_R97F

In some embodiments, a KOD polymerase variant comprises: an A at a position corresponding to P36 of SEQ ID NO: 1, a S at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a S at position P90 of SEQ ID NO: 1, a R at position V93 of SEQ ID NO: 1, and a F at position R97 of SEQ ID NO: 1. In some embodiments, a KOD polymerase variant comprises: an A at position P36 of SEQ ID NO: 1, a S at position P90 of SEQ ID NO: 1, a R at position V93 of SEQ ID NO: 1, and a F at position R97 of SEQ ID NO: 1.

### P36A_P90W

In some embodiments, a KOD polymerase variant comprises: an A at a position corresponding to P36 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a W at position P90 of SEQ ID NO: 1. In some embodiments, a KOD polymerase variant comprises: an A at position P36 of SEQ ID NO: 1, and a W at position P90 of SEQ ID NO: 1.

### P36A_P90W_R97F

In some embodiments, a KOD polymerase variant comprises: an A at a position corresponding to P36 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, and a F at position R97 of SEQ ID NO: 1. In some embodiments, a KOD polymerase variant comprises: an A at position P36 of SEQ ID NO: 1, a W at position P90 of SEQ ID NO: 1, and a F at position R97 of SEQ ID NO: 1.

### P36A_P90W_V93I

In some embodiments, a KOD polymerase variant comprises: an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, and an I at position V93 of SEQ ID NO: 1. In some embodiments, a KOD polymerase variant comprises: an A at position P36 of SEQ ID NO: 1, a W at position P90 of SEQ ID NO: 1, and an I at position V93 of SEQ ID NO: 1.

### P36A_P90W_V93I_R97A

In some embodiments, a KOD polymerase variant comprises: an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, a W at position P90 of SEQ ID NO: 1, an I at position V93 of SEQ ID NO: 1, and an A at position R97 of SEQ ID NO: 1.

### P36A_P90W_V93I_R97F

In some embodiments, a KOD polymerase variant comprises: an A at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, a W at position P90 of SEQ ID NO: 1, an I at position V93 of SEQ ID NO: 1, and a F at position R97 of SEQ ID NO: 1.

### P36A_P90W_V93I_R97H

In some embodiments, a KOD polymerase variant comprises: an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: an A at position P36 of SEQ ID NO: 1, a W at position P90 of SEQ ID NO: 1, an I at position V93 of SEQ ID NO: 1, and a H at position R97 of SEQ ID NO: 1.

### P36A_P90W_V93K_R97F

In some embodiments, a KOD polymerase variant comprises: an A at a position corresponding to P36 of SEQ ID NO: 1, a W at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, a K at position V93 of SEQ ID NO: 1, and a F at position R97 of SEQ ID NO: 1. In some embodiments, a KOD polymerase variant comprises: an A at position P36 of SEQ ID NO: 1, a W at position P90 of SEQ ID NO: 1, a K at position V93 of SEQ ID NO: 1, and a F at position R97 of SEQ ID NO: 1.

### P36A_P90W_V93K_R97W

In some embodiments, a KOD polymerase variant comprises: an A at a position corresponding to P36 of SEQ ID NO: 1, a W at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, a K at position V93 of SEQ ID NO: 1, and a W at position R97 of SEQ ID NO: 1. In some embodiments, a KOD polymerase variant comprises: an A at position P36 of SEQ ID NO: 1, a W at position P90 of SEQ ID NO: 1, a K at position V93 of SEQ ID NO: 1, and a W at position R97 of SEQ ID NO: 1.

### P36A_P90W_V93Q

In some embodiments, a KOD polymerase variant comprises: an A at a position corresponding to P36 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, and a Q at position V93 of SEQ ID NO: 1. In some embodiments, a KOD polymerase variant comprises: an A at position P36 of SEQ ID NO: 1, a W at position P90 of SEQ ID NO: 1, and a Q at position V93 of SEQ ID NO: 1.

### P36A_P90W_V93Q_R97W

In some embodiments, a KOD polymerase variant comprises: an A at a position corresponding to P36 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: an A at position P36 of SEQ ID NO: 1, a W at position P90 of SEQ ID NO: 1, a Q at position V93 of SEQ ID NO: 1, and a W at position R97 of SEQ ID NO: 1.

### P36A_P90W_V93R_R97A

In some embodiments, a KOD polymerase variant comprises: an A at a position corresponding to P36 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: an A at position P36 of SEQ ID NO: 1, a W at position P90 of SEQ ID NO: 1, a R at position V93 of SEQ ID NO: 1, and an A at position R97 of SEQ ID NO: 1.

### P36A_P90W_V93R_R97W

In some embodiments, a KOD polymerase variant comprises: an A at a position corresponding to P36 of SEQ ID NO: 1, a W at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a W at position P90 of SEQ ID NO: 1, a R at position V93 of SEQ ID NO: 1, and a W at position R97 of SEQ ID NO: 1. In some embodiments, a KOD polymerase variant comprises: an A at position P36 of SEQ ID NO: 1, a W at position P90 of SEQ ID NO: 1, a R at position V93 of SEQ ID NO: 1, and a W at position R97 of SEQ ID NO: 1.

### P36A_P90Y

In some embodiments, a KOD polymerase variant comprises: an A at a position corresponding to P36 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a Y at position P90 of SEQ ID NO: 1. In some embodiments, a KOD polymerase variant comprises: an A at position P36 of SEQ ID NO: 1, and a Y at position P90 of SEQ ID NO: 1.

### P36A_P90Y_R97F

In some embodiments, a KOD polymerase variant comprises: an A at a position corresponding to P36 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, and a F at position R97 of SEQ ID NO: 1. In some embodiments, a KOD polymerase variant comprises: an A at position P36 of SEQ ID NO: 1, a Y at position P90 of SEQ ID NO: 1, and a F at position R97 of SEQ ID NO: 1.

### P36A_P90Y_R97H

In some embodiments, a KOD polymerase variant comprises: an A at a position corresponding to P36 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, and a H at position R97 of SEQ ID NO: 1. In some embodiments, a KOD polymerase variant comprises: an A at position P36 of SEQ ID NO: 1, a Y at position P90 of SEQ ID NO: 1, and a H at position R97 of SEQ ID NO: 1.

### P36A_P90Y_V93I

In some embodiments, a KOD polymerase variant comprises: an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, and an I at position V93 of SEQ ID NO: 1. In some embodiments, a KOD polymerase variant comprises: an A at position P36 of SEQ ID NO: 1, a Y at position P90 of SEQ ID NO: 1, and an I at position V93 of SEQ ID NO: 1.

### P36A_P90Y_V93I_R97A

In some embodiments, a KOD polymerase variant comprises: an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, a Y at position P90 of SEQ ID NO: 1, an I at position V93 of SEQ ID NO: 1, and an A at position R97 of SEQ ID NO: 1.

### P36A_P90Y_V93I_R97F

In some embodiments, a KOD polymerase variant comprises: an A at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, a Y at position P90 of SEQ ID NO: 1, an I at position V93 of SEQ ID NO: 1, and a F at position R97 of SEQ ID NO: 1.

### P36A_P90Y_V93I_R97H

In some embodiments, a KOD polymerase variant comprises: an A at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: an A at position P36 of SEQ ID NO: 1, a Y at position P90 of SEQ ID NO: 1, an I at position V93 of SEQ ID NO: 1, and a H at position R97 of SEQ ID NO: 1.

### P36A_P90Y_V93R

In some embodiments, a KOD polymerase variant comprises: an A at a position corresponding to P36 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Y at position P90 of SEQ ID NO: 1, and a R at position V93 of SEQ ID NO: 1. In some embodiments, a KOD polymerase variant comprises: an A at position P36 of SEQ ID NO: 1, a Y at position P90 of SEQ ID NO: 1, and a R at position V93 of SEQ ID NO: 1.

### P36A_R97A

In some embodiments, a KOD polymerase variant comprises: an A at a position corresponding to P36 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and an A at position R97 of SEQ ID NO: 1. In some embodiments, a KOD polymerase variant comprises: an A at position P36 of SEQ ID NO: 1, and an A at position R97 of SEQ ID NO: 1.

### P36A_R97F

In some embodiments, a KOD polymerase variant comprises: an A at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a F at position R97 of SEQ ID NO: 1. In some embodiments, a KOD polymerase variant comprises: an A at position P36 of SEQ ID NO: 1, and a F at position R97 of SEQ ID NO: 1.

### P36A_V93E

In some embodiments, a KOD polymerase variant comprises: an A at a position corresponding to P36 of SEQ ID NO: 1 and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and an E at position V93 of SEQ ID NO: 1. In some embodiments, a KOD polymerase variant comprises: an A at position P36 of SEQ ID NO: 1, and an E at position V93 of SEQ ID NO: 1.

### P36A_V93I

In some embodiments, a KOD polymerase variant comprises: an A at a position corresponding to P36 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and an I at position V93 of SEQ ID NO: 1. In some embodiments, a KOD polymerase variant comprises: an A at position P36 of SEQ ID NO: 1, and an I at position V93 of SEQ ID NO: 1.

### P36A_V93I_R97A

In some embodiments, a KOD polymerase variant comprises: an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position V93 of SEQ ID NO: 1, and an A at position R97 of SEQ ID NO: 1. In some embodiments, a KOD polymerase variant comprises: an A at position P36 of SEQ ID NO: 1, an I at position V93 of SEQ ID NO: 1, and an A at position R97 of SEQ ID NO: 1.

### P36A_V93I_R97F

In some embodiments, a KOD polymerase variant comprises: an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position V93 of SEQ ID NO: 1, and a F at position R97 of SEQ ID NO: 1. In some embodiments, a KOD polymerase variant comprises: an A at position P36 of SEQ ID NO: 1, an I at position V93 of SEQ ID NO: 1, and a F at position R97 of SEQ ID NO: 1.

### P36A_V93I_R97W

In some embodiments, a KOD polymerase variant comprises: an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position V93 of SEQ ID NO: 1, and a W at position R97 of SEQ ID NO: 1. In some embodiments, a KOD polymerase variant comprises: an A at position P36 of SEQ ID NO: 1, an I at position V93 of SEQ ID NO: 1, and a W at position R97 of SEQ ID NO: 1.

### P36A_V93I_R119Q

In some embodiments, a KOD polymerase variant comprises: an A at a position corresponding to P36 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, an I at position V93 of SEQ ID NO: 1, and a Q at position R119 of SEQ ID NO: 1. In some embodiments, a KOD polymerase variant comprises: an A at position P36 of SEQ ID NO: 1, an I at position V93 of SEQ ID NO: 1, and a Q at position R119 of SEQ ID NO: 1.

### P36A_V93K

In some embodiments, a KOD polymerase variant comprises: an A at a position corresponding to P36 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a K at position V93 of SEQ ID NO: 1. In some embodiments, a KOD polymerase variant comprises: an A at position P36 of SEQ ID NO: 1, and a K at position V93 of SEQ ID NO: 1.

### P36A_V93K_R97A

In some embodiments, a KOD polymerase variant comprises: an A at a position corresponding to P36 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a K at position V93 of SEQ ID NO: 1, and an A at position R97 of SEQ ID NO: 1. In some embodiments, a KOD polymerase variant comprises: an A at position P36 of SEQ ID NO: 1, a K at position V93 of SEQ ID NO: 1, and an A at position R97 of SEQ ID NO: 1.

### P36A_V93K_R97A_R119Q

In some embodiments, a KOD polymerase variant comprises: an A at a position corresponding to P36 of SEQ ID NO: 1, a K at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a K at position V93 of SEQ ID NO: 1, an A at position R97 of SEQ ID NO: 1, and a Q at position R119 of SEQ ID NO: 1. In some embodiments, a KOD polymerase variant comprises: an A at position P36 of SEQ ID NO: 1, a K at position V93 of SEQ ID NO: 1, an A at position R97 of SEQ ID NO: 1, and a Q at position R119 of SEQ ID NO: 1.

### P36A_V93K_R97H

In some embodiments, a KOD polymerase variant comprises: an A at a position corresponding to P36 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a K at position V93 of SEQ ID NO: 1, and a H at position R97 of SEQ ID NO: 1. In some embodiments, a KOD polymerase variant comprises: an A at position P36 of SEQ ID NO: 1, a K at position V93 of SEQ ID NO: 1, and a H at position R97 of SEQ ID NO: 1.

### P36A_V93K_R119Q

In some embodiments, a KOD polymerase variant comprises: an A at a position corresponding to P36 of SEQ ID NO: 1, a K at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a K at position V93 of SEQ ID NO: 1, and a Q at position R119 of SEQ ID NO: 1. In some embodiments, a KOD polymerase variant comprises: an A at position P36 of SEQ ID NO: 1, a K at position V93 of SEQ ID NO: 1, and a Q at position R119 of SEQ ID NO: 1.

### P36A_V93Q

In some embodiments, a KOD polymerase variant comprises: an A at a position corresponding to P36 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a Q at position V93 of SEQ ID NO: 1. In some embodiments, a KOD polymerase variant comprises: an A at position P36 of SEQ ID NO: 1, and a Q at position V93 of SEQ ID NO: 1.

### P36A_V93Q_R97W

In some embodiments, a KOD polymerase variant comprises: an A at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a Q at position V93 of SEQ ID NO: 1, and a W at position R97 of SEQ ID NO: 1. In some embodiments, a KOD polymerase variant comprises: an A at position P36 of SEQ ID NO: 1, a Q at position V93 of SEQ ID NO: 1, and a W at position R97 of SEQ ID NO: 1.

### P36A_V93R

In some embodiments, a KOD polymerase variant comprises: an A at a position corresponding to P36 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, and a R at position V93 of SEQ ID NO: 1. In some embodiments, a KOD polymerase variant comprises: an A at position P36 of SEQ ID NO: 1, and a R at position V93 of SEQ ID NO: 1.

### P36A_V93R_R97A

In some embodiments, a KOD polymerase variant comprises: an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, and an A at position R97 of SEQ ID NO: 1. In some embodiments, a KOD polymerase variant comprises: an A at position P36 of SEQ ID NO: 1, a R at position V93 of SEQ ID NO: 1, and an A at position R97 of SEQ ID NO: 1.

### P36A_V93R_R97A_R119M

In some embodiments, a KOD polymerase variant comprises: an A at a position corresponding to P36 of SEQ ID NO: 1, a R at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, an A at position R97 of SEQ ID NO: 1, and a M at position R119 of SEQ ID NO: 1. In some embodiments, a KOD polymerase variant comprises: an A at position P36 of SEQ ID NO: 1, a R at position V93 of SEQ ID NO: 1, an A at position R97 of SEQ ID NO: 1, and a M at position R119 of SEQ ID NO: 1.

### P36A_V93R_R97F

In some embodiments, a KOD polymerase variant comprises: an A at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, and a F at position R97 of SEQ ID NO: 1. In some embodiments, a KOD polymerase variant comprises: an A at position P36 of SEQ ID NO: 1, a R at position V93 of SEQ ID NO: 1, and a F at position R97 of SEQ ID NO: 1.

### P36A_V93R_R97H

In some embodiments, a KOD polymerase variant comprises: an A at a position corresponding to P36 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, and a H at position R97 of SEQ ID NO: 1. In some embodiments, a KOD polymerase variant comprises: an A at position P36 of SEQ ID NO: 1, a R at position V93 of SEQ ID NO: 1, and a H at position R97 of SEQ ID NO: 1.

### P36A_V93R_R97W

In some embodiments, a KOD polymerase variant comprises: an A at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, and a W at position R97 of SEQ ID NO: 1. In some embodiments, a KOD polymerase variant comprises: an A at position P36 of SEQ ID NO: 1, a R at position V93 of SEQ ID NO: 1, and a W at position R97 of SEQ ID NO: 1.

### P36A_V93R_R119Q

In some embodiments, a KOD polymerase variant comprises: an A at a position corresponding to P36 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position P36 of SEQ ID NO: 1, a R at position V93 of SEQ ID NO: 1, and a Q at position R119 of SEQ ID NO: 1. In some embodiments, a KOD polymerase variant comprises: an A at position P36 of SEQ ID NO: 1, a R at position V93 of SEQ ID NO: 1, and a Q at position R119 of SEQ ID NO: 1.

### P36H

In some embodiments, a KOD polymerase variant comprises: a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1. In some embodiments, a KOD polymerase variant comprises: a H at position P36 of SEQ ID NO: 1.

### P36H_P90F

In some embodiments, a KOD polymerase variant comprises: a H at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a F at position P90 of SEQ ID NO: 1. In some embodiments, a KOD polymerase variant comprises: a H at position P36 of SEQ ID NO: 1, and a F at position P90 of SEQ ID NO: 1.

### P36H_P90F_R97F

In some embodiments, a KOD polymerase variant comprises: a H at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, and a F at position R97 of SEQ ID NO: 1. In some embodiments, a KOD polymerase variant comprises: a H at position P36 of SEQ ID NO: 1, a F at position P90 of SEQ ID NO: 1, and a F at position R97 of SEQ ID NO: 1.

### P36H_P90F_V93I

In some embodiments, a KOD polymerase variant comprises: a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, and an I at position V93 of SEQ ID NO: 1. In some embodiments, a KOD polymerase variant comprises: a H at position P36 of SEQ ID NO: 1, a F at position P90 of SEQ ID NO: 1, and an I at position V93 of SEQ ID NO: 1.

### P36H_P90F_V93I_R97A

In some embodiments, a KOD polymerase variant comprises: a H at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a H at position P36 of SEQ ID NO: 1, a F at position P90 of SEQ ID NO: 1, an I at position V93 of SEQ ID NO: 1, and an A at position R97 of SEQ ID NO: 1.

### P36H_P90F_V93I_R97W

In some embodiments, a KOD polymerase variant comprises: a H at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a H at position P36 of SEQ ID NO: 1, a F at position P90 of SEQ ID NO: 1, an I at position V93 of SEQ ID NO: 1, and a W at position R97 of SEQ ID NO: 1.

### P36H_P90I

In some embodiments, a KOD polymerase variant comprises: a H at a position corresponding to P36 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and an I at position P90 of SEQ ID NO: 1. In some embodiments, a KOD polymerase variant comprises: a H at position P36 of SEQ ID NO: 1, and an I at position P90 of SEQ ID NO: 1.

### P36H_P90I_R97A

In some embodiments, a KOD polymerase variant comprises: a H at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, and an A at position R97 of SEQ ID NO: 1. In some embodiments, a KOD polymerase variant comprises: a H at position P36 of SEQ ID NO: 1, an I at position P90 of SEQ ID NO: 1, and an A at position R97 of SEQ ID NO: 1.

### P36H_P90I_V93I

In some embodiments, a KOD polymerase variant comprises: a H at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, and an I at position V93 of SEQ ID NO: 1. In some embodiments, a KOD polymerase variant comprises: a H at position P36 of SEQ ID NO: 1, an I at position P90 of SEQ ID NO: 1, and an I at position V93 of SEQ ID NO: 1.

### P36H_P90I_V93I_R97H

In some embodiments, a KOD polymerase variant comprises: a H at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a H at position P36 of SEQ ID NO: 1, an I at position P90 of SEQ ID NO: 1, an I at position V93 of SEQ ID NO: 1, and a H at position R97 of SEQ ID NO: 1.

### P36H_P90I_V93I_R97W

In some embodiments, a KOD polymerase variant comprises: a H at a position corresponding to P36 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a H at position P36 of SEQ ID NO: 1, an I at position P90 of SEQ ID NO: 1, an I at position V93 of SEQ ID NO: 1, and a W at position R97 of SEQ ID NO: 1.

### P36H_P90I_V93K_R97A

In some embodiments, a KOD polymerase variant comprises: a H at a position corresponding to P36 of SEQ ID NO: 1, an I at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, a K at position V93 of SEQ ID NO: 1, and an A at position R97 of SEQ ID NO: 1. In some embodiments, a KOD polymerase variant comprises: a H at position P36 of SEQ ID NO: 1, an I at position P90 of SEQ ID NO: 1, a K at position V93 of SEQ ID NO: 1, and an A at position R97 of SEQ ID NO: 1.

### P36H_P90I_V93R_R97F

In some embodiments, a KOD polymerase variant comprises: a H at a position corresponding to P36 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a H at position P36 of SEQ ID NO: 1, an I at position P90 of SEQ ID NO: 1, a R at position V93 of SEQ ID NO: 1, and a F at position R97 of SEQ ID NO: 1.

### P36H_P90S_R97A

In some embodiments, a KOD polymerase variant comprises: a H at a position corresponding to P36 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a S at position P90 of SEQ ID NO: 1, and an A at position R97 of SEQ ID NO: 1. In some embodiments, a KOD polymerase variant comprises: a H at position P36 of SEQ ID NO: 1, a S at position P90 of SEQ ID NO: 1, and an A at position R97 of SEQ ID NO: 1.

### P36H_P90S_V93I

In some embodiments, a KOD polymerase variant comprises: a H at a position corresponding to P36 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a S at position P90 of SEQ ID NO: 1, and an I at position V93 of SEQ ID NO: 1. In some embodiments, a KOD polymerase variant comprises: a H at position P36 of SEQ ID NO: 1, a S at position P90 of SEQ ID NO: 1, and an I at position V93 of SEQ ID NO: 1.

### P36H_P90S_V93I_R97A

In some embodiments, a KOD polymerase variant comprises: a H at a position corresponding to P36 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a H at position P36 of SEQ ID NO: 1, a S at position P90 of SEQ ID NO: 1, an I at position V93 of SEQ ID NO: 1, and an A at position R97 of SEQ ID NO: 1.

### P36H_P90S_V93I_R97W

In some embodiments, a KOD polymerase variant comprises: a H at a position corresponding to P36 of SEQ ID NO: 1, a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a S at position P90 of SEQ ID NO: 1, an I at position V93 of SEQ ID NO: 1, and a W at position R97 of SEQ ID NO: 1. In some embodiments, a KOD polymerase variant comprises: a H at position P36 of SEQ ID NO: 1, a S at position P90 of SEQ ID NO: 1, an I at position V93 of SEQ ID NO: 1, and a W at position R97 of SEQ ID NO: 1.

### P36H_P90W

In some embodiments, a KOD polymerase variant comprises: a H at a position corresponding to P36 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a W at position P90 of SEQ ID NO: 1. In some embodiments, a KOD polymerase variant comprises: a H at position P36 of SEQ ID NO: 1, and a W at position P90 of SEQ ID NO: 1.

### P36H_P90W_R97F

In some embodiments, a KOD polymerase variant comprises: a H at a position corresponding to P36 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a W at position P90 of SEQ ID NO: 1, and a F at position R97 of SEQ ID NO: 1. In some embodiments, a KOD polymerase variant comprises: a H at position P36 of SEQ ID NO: 1, a W at position P90 of SEQ ID NO: 1, and a F at position R97 of SEQ ID NO: 1.

### P36H_P90W_R97H

In some embodiments, a KOD polymerase variant comprises: a H at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a W at position P90 of SEQ ID NO: 1, and a H at position R97 of SEQ ID NO: 1. In some embodiments, a KOD polymerase variant comprises: a H at position P36 of SEQ ID NO: 1, a W at position P90 of SEQ ID NO: 1, and a H at position R97 of SEQ ID NO: 1.

### P36H_P90W_R97W

In some embodiments, a KOD polymerase variant comprises: a H at a position corresponding to P36 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a W at position P90 of SEQ ID NO: 1, and a W at position R97 of SEQ ID NO: 1. In some embodiments, a KOD polymerase variant comprises: a H at position P36 of SEQ ID NO: 1, a W at position P90 of SEQ ID NO: 1, and a W at position R97 of SEQ ID NO: 1.

### P36H_P90W_V93I

In some embodiments, a KOD polymerase variant comprises: a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a W at position P90 of SEQ ID NO: 1, and an I at position V93 of SEQ ID NO: 1. In some embodiments, a KOD polymerase variant comprises: a H at position P36 of SEQ ID NO: 1, a W at position P90 of SEQ ID NO: 1, and an I at position V93 of SEQ ID NO: 1.

### P36H_P90W_V93I_R97H

In some embodiments, a KOD polymerase variant comprises: a H at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a H at position P36 of SEQ ID NO: 1, a W at position P90 of SEQ ID NO: 1, an I at position V93 of SEQ ID NO: 1, and a H at position R97 of SEQ ID NO: 1.

### P36H_P90Y

In some embodiments, a KOD polymerase variant comprises: a H at a position corresponding to P36 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a Y at position P90 of SEQ ID NO: 1. In some embodiments, a KOD polymerase variant comprises: a H at position P36 of SEQ ID NO: 1, and a Y at position P90 of SEQ ID NO: 1.

### P36H_P90Y_V93I

In some embodiments, a KOD polymerase variant comprises: a H at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a Y at position P90 of SEQ ID NO: 1, and an I at position V93 of SEQ ID NO: 1. In some embodiments, a KOD polymerase variant comprises: a H at position P36 of SEQ ID NO: 1, a Y at position P90 of SEQ ID NO: 1, and an I at position V93 of SEQ ID NO: 1.

### P36H_P90Y_V93I_R97A

In some embodiments, a KOD polymerase variant comprises: a H at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a H at position P36 of SEQ ID NO: 1, a Y at position P90 of SEQ ID NO: 1, an I at position V93 of SEQ ID NO: 1, and an A at position R97 of SEQ ID NO: 1.

### P36H_R97A

In some embodiments, a KOD polymerase variant comprises: a H at a position corresponding to P36 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and an A at position R97 of SEQ ID NO: 1. In some embodiments, a KOD polymerase variant comprises: a H at position P36 of SEQ ID NO: 1, and an A at position R97 of SEQ ID NO: 1.

### P36H_R97F

In some embodiments, a KOD polymerase variant comprises: a H at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a F at position R97 of SEQ ID NO: 1. In some embodiments, a KOD polymerase variant comprises: a H at position P36 of SEQ ID NO: 1, and a F at position R97 of SEQ ID NO: 1.

### P36H_R97H

In some embodiments, a KOD polymerase variant comprises: a H at a position corresponding to P36 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a H at position R97 of SEQ ID NO: 1. In some embodiments, a KOD polymerase variant comprises: a H at position P36 of SEQ ID NO: 1, and a H at position R97 of SEQ ID NO: 1.

### P36H_R97W

In some embodiments, a KOD polymerase variant comprises: a H at a position corresponding to P36 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a W at position R97 of SEQ ID NO: 1. In some embodiments, a KOD polymerase variant comprises: a H at position P36 of SEQ ID NO: 1, and a W at position R97 of SEQ ID NO: 1.

### P36H_V93I

In some embodiments, a KOD polymerase variant comprises: a H at a position corresponding to P36 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and an I at position V93 of SEQ ID NO: 1. In some embodiments, a KOD polymerase variant comprises: a H at position P36 of SEQ ID NO: 1, and an I at position V93 of SEQ ID NO: 1.

### P36H_V93I_R97A

In some embodiments, a KOD polymerase variant comprises: a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position V93 of SEQ ID NO: 1, and an A at position R97 of SEQ ID NO: 1. In some embodiments, a KOD polymerase variant comprises: a H at position P36 of SEQ ID NO: 1, an I at position V93 of SEQ ID NO: 1, and an A at position R97 of SEQ ID NO: 1.

### P36H_V93I_R97F

In some embodiments, a KOD polymerase variant comprises: a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position V93 of SEQ ID NO: 1, and a F at position R97 of SEQ ID NO: 1. In some embodiments, a KOD polymerase variant comprises: a H at position P36 of SEQ ID NO: 1, an I at position V93 of SEQ ID NO: 1, and a F at position R97 of SEQ ID NO: 1.

### P36H_V93I_R97H

In some embodiments, a KOD polymerase variant comprises: a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position V93 of SEQ ID NO: 1, and a H at position R97 of SEQ ID NO: 1. In some embodiments, a KOD polymerase variant comprises: a H at position P36 of SEQ ID NO: 1, an I at position V93 of SEQ ID NO: 1, and a H at position R97 of SEQ ID NO: 1.

### P36H_V93I_R97W

In some embodiments, a KOD polymerase variant comprises: a H at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, an I at position V93 of SEQ ID NO: 1, and a W at position R97 of SEQ ID NO: 1. In some embodiments, a KOD polymerase variant comprises: a H at position P36 of SEQ ID NO: 1, an I at position V93 of SEQ ID NO: 1, and a W at position R97 of SEQ ID NO: 1.

### P36H_V93K

In some embodiments, a KOD polymerase variant comprises: a H at a position corresponding to P36 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a K at position V93 of SEQ ID NO: 1. In some embodiments, a KOD polymerase variant comprises: a H at position P36 of SEQ ID NO: 1, and a K at position V93 of SEQ ID NO: 1.

### P36H_V93K_R97H

In some embodiments, a KOD polymerase variant comprises: a H at a position corresponding to P36 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, and a H at position R97 of SEQ ID NO: 1. In some embodiments, a KOD polymerase variant comprises: a H at position P36 of SEQ ID NO: 1, a K at position V93 of SEQ ID NO: 1, and a H at position R97 of SEQ ID NO: 1.

### P36H_V93Q

In some embodiments, a KOD polymerase variant comprises: a H at a position corresponding to P36 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a Q at position V93 of SEQ ID NO: 1. In some embodiments, a KOD polymerase variant comprises: a H at position P36 of SEQ ID NO: 1, and a Q at position V93 of SEQ ID NO: 1.

### P36H_V93Q_R97W

In some embodiments, a KOD polymerase variant comprises: a H at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a Q at position V93 of SEQ ID NO: 1, and a W at position R97 of SEQ ID NO: 1. In some embodiments, a KOD polymerase variant comprises: a H at position P36 of SEQ ID NO: 1, a Q at position V93 of SEQ ID NO: 1, and a W at position R97 of SEQ ID NO: 1.

### P36H_V93R

In some embodiments, a KOD polymerase variant comprises: a H at a position corresponding to P36 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a R at position V93 of SEQ ID NO: 1. In some embodiments, a KOD polymerase variant comprises: a H at position P36 of SEQ ID NO: 1, and a R at position V93 of SEQ ID NO: 1.

### P36H_V93R_R97A

In some embodiments, a KOD polymerase variant comprises: a H at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a R at position V93 of SEQ ID NO: 1, and an A at position R97 of SEQ ID NO: 1. In some embodiments, a KOD polymerase variant comprises: a H at position P36 of SEQ ID NO: 1, a R at position V93 of SEQ ID NO: 1, and an A at position R97 of SEQ ID NO: 1.

### P36H_V93R_R97F

In some embodiments, a KOD polymerase variant comprises: a H at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a R at position V93 of SEQ ID NO: 1, and a F at position R97 of SEQ ID NO: 1. In some embodiments, a KOD polymerase variant comprises: a H at position P36 of SEQ ID NO: 1, a R at position V93 of SEQ ID NO: 1, and a F at position R97 of SEQ ID NO: 1.

### P36H_V93R_R97W

In some embodiments, a KOD polymerase variant comprises: a H at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a R at position V93 of SEQ ID NO: 1, and a W at position R97 of SEQ ID NO: 1. In some embodiments, a KOD polymerase variant comprises: a H at position P36 of SEQ ID NO: 1, a R at position V93 of SEQ ID NO: 1, and a W at position R97 of SEQ ID NO: 1.

### P36R

In some embodiments, a KOD polymerase variant comprises: a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1. In some embodiments, a KOD polymerase variant comprises: a R at position P36 of SEQ ID NO: 1.

### P36R_P90F

In some embodiments, a KOD polymerase variant comprises: a R at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and a F at position P90 of SEQ ID NO: 1. In some embodiments, a KOD polymerase variant comprises: a R at position P36 of SEQ ID NO: 1, and a F at position P90 of SEQ ID NO: 1.

### P36R_P90I

In some embodiments, a KOD polymerase variant comprises: a R at a position corresponding to P36 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and an I at position P90 of SEQ ID NO: 1. In some embodiments, a KOD polymerase variant comprises: a R at position P36 of SEQ ID NO: 1, and an I at position P90 of SEQ ID NO: 1.

### P36R_P90I_V93I

In some embodiments, a KOD polymerase variant comprises: a R at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position P90 of SEQ ID NO: 1, and an I at position V93 of SEQ ID NO: 1. In some embodiments, a KOD polymerase variant comprises: a R at position P36 of SEQ ID NO: 1, an I at position P90 of SEQ ID NO: 1, and an I at position V93 of SEQ ID NO: 1.

### P36R_P90I_V93Q

In some embodiments, a KOD polymerase variant comprises: a R at a position corresponding to P36 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position P90 of SEQ ID NO: 1, and a Q at position V93 of SEQ ID NO: 1. In some embodiments, a KOD polymerase variant comprises: a R at position P36 of SEQ ID NO: 1, an I at position P90 of SEQ ID NO: 1, and a Q at position V93 of SEQ ID NO: 1.

### P36R_P90S_V93I_R97F

In some embodiments, a KOD polymerase variant comprises: a R at a position corresponding to P36 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: a R at position P36 of SEQ ID NO: 1, a S at position P90 of SEQ ID NO: 1, an I at position V93 of SEQ ID NO: 1, and a F at position R97 of SEQ ID NO: 1.

### P36R_P90Y_V93I

In some embodiments, a KOD polymerase variant comprises: a R at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, a Y at position P90 of SEQ ID NO: 1, and an I at position V93 of SEQ ID NO: 1. In some embodiments, a KOD polymerase variant comprises: a R at position P36 of SEQ ID NO: 1, a Y at position P90 of SEQ ID NO: 1, and an I at position V93 of SEQ ID NO: 1.

### P36R_R97A

In some embodiments, a KOD polymerase variant comprises: a R at a position corresponding to P36 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and an A at position R97 of SEQ ID NO: 1. In some embodiments, a KOD polymerase variant comprises: a R at position P36 of SEQ ID NO: 1, and an A at position R97 of SEQ ID NO: 1.

### P36R_R97F

In some embodiments, a KOD polymerase variant comprises: a R at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and a F at position R97 of SEQ ID NO: 1. In some embodiments, a KOD polymerase variant comprises: a R at position P36 of SEQ ID NO: 1, and a F at position R97 of SEQ ID NO: 1.

### P36R_R97W

In some embodiments, a KOD polymerase variant comprises: a R at a position corresponding to P36 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and a W at position R97 of SEQ ID NO: 1. In some embodiments, a KOD polymerase variant comprises: a R at position P36 of SEQ ID NO: 1, and a W at position R97 of SEQ ID NO: 1.

### P36R_R119Q

In some embodiments, a KOD polymerase variant comprises: a R at a position corresponding to P36 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and a Q at position R119 of SEQ ID NO: 1. In some embodiments, a KOD polymerase variant comprises: a R at position P36 of SEQ ID NO: 1, and a Q at position R119 of SEQ ID NO: 1.

### P36R_V93E

In some embodiments, a KOD polymerase variant comprises: a R at a position corresponding to P36 of SEQ ID NO: 1 and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and an E at position V93 of SEQ ID NO: 1. In some embodiments, a KOD polymerase variant comprises: a R at position P36 of SEQ ID NO: 1, and an E at position V93 of SEQ ID NO: 1.

### P36R_V93I

In some embodiments, a KOD polymerase variant comprises: a R at a position corresponding to P36 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, and an I at position V93 of SEQ ID NO: 1. In some embodiments, a KOD polymerase variant comprises: a R at position P36 of SEQ ID NO: 1, and an I at position V93 of SEQ ID NO: 1.

### P36R_V93I_R97A

In some embodiments, a KOD polymerase variant comprises: a R at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position V93 of SEQ ID NO: 1, and an A at position R97 of SEQ ID NO: 1. In some embodiments, a KOD polymerase variant comprises: a R at position P36 of SEQ ID NO: 1, an I at position V93 of SEQ ID NO: 1, and an A at position R97 of SEQ ID NO: 1.

### P36R_V93I_R97F

In some embodiments, a KOD polymerase variant comprises: a R at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position V93 of SEQ ID NO: 1, and a F at position R97 of SEQ ID NO: 1. In some embodiments, a KOD polymerase variant comprises: a R at position P36 of SEQ ID NO: 1, an I at position V93 of SEQ ID NO: 1, and a F at position R97 of SEQ ID NO: 1.

### P36R_V93I_R97H

In some embodiments, a KOD polymerase variant comprises: a R at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position V93 of SEQ ID NO: 1, and a H at position R97 of SEQ ID NO: 1. In some embodiments, a KOD polymerase variant comprises: a R at position P36 of SEQ ID NO: 1, an I at position V93 of SEQ ID NO: 1, and a H at position R97 of SEQ ID NO: 1.

### P36R_V93I_R97W

In some embodiments, a KOD polymerase variant comprises: a R at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position P36 of SEQ ID NO: 1, an I at position V93 of SEQ ID NO: 1, and a W at position R97 of SEQ ID NO: 1. In some embodiments, a KOD polymerase variant comprises: a R at position P36 of SEQ ID NO: 1, an I at position V93 of SEQ ID NO: 1, and a W at position R97 of SEQ ID NO: 1.

### P36S

In some embodiments, a KOD polymerase variant comprises: a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1. In some embodiments, a KOD polymerase variant comprises: a S at position P36 of SEQ ID NO: 1.

### P36S_P90F

In some embodiments, a KOD polymerase variant comprises: a S at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a F at position P90 of SEQ ID NO: 1. In some embodiments, a KOD polymerase variant comprises: a S at position P36 of SEQ ID NO: 1, and a F at position P90 of SEQ ID NO: 1.

### P36S_P90F_V93I

In some embodiments, a KOD polymerase variant comprises: a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a F at position P90 of SEQ ID NO: 1, and an I at position V93 of SEQ ID NO: 1. In some embodiments, a KOD polymerase variant comprises: a S at position P36 of SEQ ID NO: 1, a F at position P90 of SEQ ID NO: 1, and an I at position V93 of SEQ ID NO: 1.

### P36S_P90F_V93I_R97A

In some embodiments, a KOD polymerase variant comprises: a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a S at position P36 of SEQ ID NO: 1, a F at position P90 of SEQ ID NO: 1, an I at position V93 of SEQ ID NO: 1, and an A at position R97 of SEQ ID NO: 1.

### P36S_P90F_V93I_R97F

In some embodiments, a KOD polymerase variant comprises: a S at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a S at position P36 of SEQ ID NO: 1, a F at position P90 of SEQ ID NO: 1, an I at position V93 of SEQ ID NO: 1, and a F at position R97 of SEQ ID NO: 1.

### P36S_P90F_V93I_R97H

In some embodiments, a KOD polymerase variant comprises: a S at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a S at position P36 of SEQ ID NO: 1, a F at position P90 of SEQ ID NO: 1, an I at position V93 of SEQ ID NO: 1, and a H at position R97 of SEQ ID NO: 1.

### P36S_P90F_V93I_R97W

In some embodiments, a KOD polymerase variant comprises: a S at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a S at position P36 of SEQ ID NO: 1, a F at position P90 of SEQ ID NO: 1, an I at position V93 of SEQ ID NO: 1, and a W at position R97 of SEQ ID NO: 1.

### P36S_P90I

In some embodiments, a KOD polymerase variant comprises: a S at a position corresponding to P36 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and an I at position P90 of SEQ ID NO: 1. In some embodiments, a KOD polymerase variant comprises: a S at position P36 of SEQ ID NO: 1, and an I at position P90 of SEQ ID NO: 1.

### P36S_P90I_R97H

In some embodiments, a KOD polymerase variant comprises: a S at a position corresponding to P36 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position P90 of SEQ ID NO: 1, and a H at position R97 of SEQ ID NO: 1. In some embodiments, a KOD polymerase variant comprises: a S at position P36 of SEQ ID NO: 1, an I at position P90 of SEQ ID NO: 1, and a H at position R97 of SEQ ID NO: 1.

### P36S_P90I_V93I

In some embodiments, a KOD polymerase variant comprises: a S at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position P90 of SEQ ID NO: 1, and an I at position V93 of SEQ ID NO: 1. In some embodiments, a KOD polymerase variant comprises: a S at position P36 of SEQ ID NO: 1, an I at position P90 of SEQ ID NO: 1, and an I at position V93 of SEQ ID NO: 1.

### P36S_P90I_V93I_R97F

In some embodiments, a KOD polymerase variant comprises: a S at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a S at position P36 of SEQ ID NO: 1, an I at position P90 of SEQ ID NO: 1, an I at position V93 of SEQ ID NO: 1, and a F at position R97 of SEQ ID NO: 1.

### P36S_P90I_V93Q

In some embodiments, a KOD polymerase variant comprises: a S at a position corresponding to P36 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position P90 of SEQ ID NO: 1, and a Q at position V93 of SEQ ID NO: 1. In some embodiments, a KOD polymerase variant comprises: a S at position P36 of SEQ ID NO: 1, an I at position P90 of SEQ ID NO: 1, and a Q at position V93 of SEQ ID NO: 1.

### P36S_P90I_V93R

In some embodiments, a KOD polymerase variant comprises: a S at a position corresponding to P36 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position P90 of SEQ ID NO: 1, and a R at position V93 of SEQ ID NO: 1. In some embodiments, a KOD polymerase variant comprises: a S at position P36 of SEQ ID NO: 1, an I at position P90 of SEQ ID NO: 1, and a R at position V93 of SEQ ID NO: 1.

### P36S_P90S_V93R_R97F

In some embodiments, a KOD polymerase variant comprises: a S at a position corresponding to P36 of SEQ ID NO: 1, a S at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a S at position P90 of SEQ ID NO: 1, a R at position V93 of SEQ ID NO: 1, and a F at position R97 of SEQ ID NO: 1. In some embodiments, a KOD polymerase variant comprises: a S at position P36 of SEQ ID NO: 1, a S at position P90 of SEQ ID NO: 1, a R at position V93 of SEQ ID NO: 1, and a F at position R97 of SEQ ID NO: 1.

### P36S_P90W

In some embodiments, a KOD polymerase variant comprises: a S at a position corresponding to P36 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a W at position P90 of SEQ ID NO: 1. In some embodiments, a KOD polymerase variant comprises: a S at position P36 of SEQ ID NO: 1, and a W at position P90 of SEQ ID NO: 1.

### P36S_P90W_R97A

In some embodiments, a KOD polymerase variant comprises: a S at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a W at position P90 of SEQ ID NO: 1, and an A at position R97 of SEQ ID NO: 1. In some embodiments, a KOD polymerase variant comprises: a S at position P36 of SEQ ID NO: 1, a W at position P90 of SEQ ID NO: 1, and an A at position R97 of SEQ ID NO: 1.

### P36S_P90W_V93I

In some embodiments, a KOD polymerase variant comprises: a S at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a W at position P90 of SEQ ID NO: 1, and an I at position V93 of SEQ ID NO: 1. In some embodiments, a KOD polymerase variant comprises: a S at position P36 of SEQ ID NO: 1, a W at position P90 of SEQ ID NO: 1, and an I at position V93 of SEQ ID NO: 1.

### P36S_P90W_V93I_R97A

In some embodiments, a KOD polymerase variant comprises: a S at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a S at position P36 of SEQ ID NO: 1, a W at position P90 of SEQ ID NO: 1, an I at position V93 of SEQ ID NO: 1, and an A at position R97 of SEQ ID NO: 1.

### P36S_P90W_V93I_R97H

In some embodiments, a KOD polymerase variant comprises: a S at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a S at position P36 of SEQ ID NO: 1, a W at position P90 of SEQ ID NO: 1, an I at position V93 of SEQ ID NO: 1, and a H at position R97 of SEQ ID NO: 1.

### P36S_P90W_V93I_R97W

In some embodiments, a KOD polymerase variant comprises: a S at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a S at position P36 of SEQ ID NO: 1, a W at position P90 of SEQ ID NO: 1, an I at position V93 of SEQ ID NO: 1, and a W at position R97 of SEQ ID NO: 1.

### P36S_P90Y

In some embodiments, a KOD polymerase variant comprises: a S at a position corresponding to P36 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a Y at position P90 of SEQ ID NO: 1. In some embodiments, a KOD polymerase variant comprises: a S at position P36 of SEQ ID NO: 1, and a Y at position P90 of SEQ ID NO: 1.

### P36S_P90Y_V93I

In some embodiments, a KOD polymerase variant comprises: a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Y at position P90 of SEQ ID NO: 1, and an I at position V93 of SEQ ID NO: 1. In some embodiments, a KOD polymerase variant comprises: a S at position P36 of SEQ ID NO: 1, a Y at position P90 of SEQ ID NO: 1, and an I at position V93 of SEQ ID NO: 1.

### P36S_P90Y_V93I_R97A

In some embodiments, a KOD polymerase variant comprises: a S at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a S at position P36 of SEQ ID NO: 1, a Y at position P90 of SEQ ID NO: 1, an I at position V93 of SEQ ID NO: 1, and an A at position R97 of SEQ ID NO: 1.

### P36S_P90Y_V93I_R97H

In some embodiments, a KOD polymerase variant comprises: a S at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a S at position P36 of SEQ ID NO: 1, a Y at position P90 of SEQ ID NO: 1, an I at position V93 of SEQ ID NO: 1, and a H at position R97 of SEQ ID NO: 1.

### P36S_P90Y_V93Q

In some embodiments, a KOD polymerase variant comprises: a S at a position corresponding to P36 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Y at position P90 of SEQ ID NO: 1, and a Q at position V93 of SEQ ID NO: 1. In some embodiments, a KOD polymerase variant comprises: a S at position P36 of SEQ ID NO: 1, a Y at position P90 of SEQ ID NO: 1, and a Q at position V93 of SEQ ID NO: 1.

### P36S_P90Y_V93R_R97A

In some embodiments, a KOD polymerase variant comprises: a S at a position corresponding to P36 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a S at position P36 of SEQ ID NO: 1, a Y at position P90 of SEQ ID NO: 1, a R at position V93 of SEQ ID NO: 1, and an A at position R97 of SEQ ID NO: 1.

### P36S_R97A

In some embodiments, a KOD polymerase variant comprises: a S at a position corresponding to P36 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and an A at position R97 of SEQ ID NO: 1. In some embodiments, a KOD polymerase variant comprises: a S at position P36 of SEQ ID NO: 1, and an A at position R97 of SEQ ID NO: 1.

### P36S_R97F

In some embodiments, a KOD polymerase variant comprises: a S at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a F at position R97 of SEQ ID NO: 1. In some embodiments, a KOD polymerase variant comprises: a S at position P36 of SEQ ID NO: 1, and a F at position R97 of SEQ ID NO: 1.

### P36S_V93I

In some embodiments, a KOD polymerase variant comprises: a S at a position corresponding to P36 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and an I at position V93 of SEQ ID NO: 1. In some embodiments, a KOD polymerase variant comprises: a S at position P36 of SEQ ID NO: 1, and an I at position V93 of SEQ ID NO: 1.

### P36S_V93I_R97F

In some embodiments, a KOD polymerase variant comprises: a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position V93 of SEQ ID NO: 1, and a F at position R97 of SEQ ID NO: 1. In some embodiments, a KOD polymerase variant comprises: a S at position P36 of SEQ ID NO: 1, an I at position V93 of SEQ ID NO: 1, and a F at position R97 of SEQ ID NO: 1.

### P36S_V93I_R119Q

In some embodiments, a KOD polymerase variant comprises: a S at a position corresponding to P36 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, an I at position V93 of SEQ ID NO: 1, and a Q at position R119 of SEQ ID NO: 1. In some embodiments, a KOD polymerase variant comprises: a S at position P36 of SEQ ID NO: 1, an I at position V93 of SEQ ID NO: 1, and a Q at position R119 of SEQ ID NO: 1.

### P36S_V93K

In some embodiments, a KOD polymerase variant comprises: a S at a position corresponding to P36 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a K at position V93 of SEQ ID NO: 1. In some embodiments, a KOD polymerase variant comprises: a S at position P36 of SEQ ID NO: 1, and a K at position V93 of SEQ ID NO: 1.

### P36S_V93K_R97A

In some embodiments, a KOD polymerase variant comprises: a S at a position corresponding to P36 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a K at position V93 of SEQ ID NO: 1, and an A at position R97 of SEQ ID NO: 1. In some embodiments, a KOD polymerase variant comprises: a S at position P36 of SEQ ID NO: 1, a K at position V93 of SEQ ID NO: 1, and an A at position R97 of SEQ ID NO: 1.

### P36S_V93Q

In some embodiments, a KOD polymerase variant comprises: a S at a position corresponding to P36 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a Q at position V93 of SEQ ID NO: 1. In some embodiments, a KOD polymerase variant comprises: a S at position P36 of SEQ ID NO: 1, and a Q at position V93 of SEQ ID NO: 1.

### P36S_V93Q_R97A

In some embodiments, a KOD polymerase variant comprises: a S at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Q at position V93 of SEQ ID NO: 1, and an A at position R97 of SEQ ID NO: 1. In some embodiments, a KOD polymerase variant comprises: a S at position P36 of SEQ ID NO: 1, a Q at position V93 of SEQ ID NO: 1, and an A at position R97 of SEQ ID NO: 1.

### P36S_V93Q_R97W

In some embodiments, a KOD polymerase variant comprises: a S at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a Q at position V93 of SEQ ID NO: 1, and a W at position R97 of SEQ ID NO: 1. In some embodiments, a KOD polymerase variant comprises: a S at position P36 of SEQ ID NO: 1, a Q at position V93 of SEQ ID NO: 1, and a W at position R97 of SEQ ID NO: 1.

### P36S_V93R

In some embodiments, a KOD polymerase variant comprises: a S at a position corresponding to P36 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, and a R at position V93 of SEQ ID NO: 1. In some embodiments, a KOD polymerase variant comprises: a S at position P36 of SEQ ID NO: 1, and a R at position V93 of SEQ ID NO: 1.

### P36S_V93R_R97A

In some embodiments, a KOD polymerase variant comprises: a S at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a R at position V93 of SEQ ID NO: 1, and an A at position R97 of SEQ ID NO: 1. In some embodiments, a KOD polymerase variant comprises: a S at position P36 of SEQ ID NO: 1, a R at position V93 of SEQ ID NO: 1, and an A at position R97 of SEQ ID NO: 1.

### P36S_V93R_R97W

In some embodiments, a KOD polymerase variant comprises: a S at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P36 of SEQ ID NO: 1, a R at position V93 of SEQ ID NO: 1, and a W at position R97 of SEQ ID NO: 1. In some embodiments, a KOD polymerase variant comprises: a S at position P36 of SEQ ID NO: 1, a R at position V93 of SEQ ID NO: 1, and a W at position R97 of SEQ ID NO: 1.

### P36W

In some embodiments, a KOD polymerase variant comprises: a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1. In some embodiments, a KOD polymerase variant comprises: a W at position P36 of SEQ ID NO: 1.

### P36W_P90F

In some embodiments, a KOD polymerase variant comprises: a W at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a F at position P90 of SEQ ID NO: 1. In some embodiments, a KOD polymerase variant comprises: a W at position P36 of SEQ ID NO: 1, and a F at position P90 of SEQ ID NO: 1.

### P36W_P90F_R97A

In some embodiments, a KOD polymerase variant comprises: a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and an A at position R97 of SEQ ID NO: 1. In some embodiments, a KOD polymerase variant comprises: a W at position P36 of SEQ ID NO: 1, a F at position P90 of SEQ ID NO: 1, and an A at position R97 of SEQ ID NO: 1.

### P36W_P90F_R97F

In some embodiments, a KOD polymerase variant comprises: a W at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and a F at position R97 of SEQ ID NO: 1. In some embodiments, a KOD polymerase variant comprises: a W at position P36 of SEQ ID NO: 1, a F at position P90 of SEQ ID NO: 1, and a F at position R97 of SEQ ID NO: 1.

### P36W_P90F_R97H

In some embodiments, a KOD polymerase variant comprises: a W at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and a H at position R97 of SEQ ID NO: 1. In some embodiments, a KOD polymerase variant comprises: a W at position P36 of SEQ ID NO: 1, a F at position P90 of SEQ ID NO: 1, and a H at position R97 of SEQ ID NO: 1.

### P36W_P90F_V93I

In some embodiments, a KOD polymerase variant comprises: a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and an I at position V93 of SEQ ID NO: 1. In some embodiments, a KOD polymerase variant comprises: a W at position P36 of SEQ ID NO: 1, a F at position P90 of SEQ ID NO: 1, and an I at position V93 of SEQ ID NO: 1.

### P36W_P90F_V93I_R97A

In some embodiments, a KOD polymerase variant comprises: a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a W at position P36 of SEQ ID NO: 1, a F at position P90 of SEQ ID NO: 1, an I at position V93 of SEQ ID NO: 1, and an A at position R97 of SEQ ID NO: 1.

### P36W_P90F_V93I_R97F

In some embodiments, a KOD polymerase variant comprises: a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a W at position P36 of SEQ ID NO: 1, a F at position P90 of SEQ ID NO: 1, an I at position V93 of SEQ ID NO: 1, and a F at position R97 of SEQ ID NO: 1.

### P36W_P90F_V93I_R97W

In some embodiments, a KOD polymerase variant comprises: a W at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a W at position P36 of SEQ ID NO: 1, a F at position P90 of SEQ ID NO: 1, an I at position V93 of SEQ ID NO: 1, and a W at position R97 of SEQ ID NO: 1.

### P36W_P90F_V93K

In some embodiments, a KOD polymerase variant comprises: a W at a position corresponding to P36 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and a K at position V93 of SEQ ID NO: 1. In some embodiments, a KOD polymerase variant comprises: a W at position P36 of SEQ ID NO: 1, a F at position P90 of SEQ ID NO: 1, and a K at position V93 of SEQ ID NO: 1.

### P36W_P90F_V93R

In some embodiments, a KOD polymerase variant comprises: a W at a position corresponding to P36 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a F at position P90 of SEQ ID NO: 1, and a R at position V93 of SEQ ID NO: 1. In some embodiments, a KOD polymerase variant comprises: a W at position P36 of SEQ ID NO: 1, a F at position P90 of SEQ ID NO: 1, and a R at position V93 of SEQ ID NO: 1.

### P36W_P90I

In some embodiments, a KOD polymerase variant comprises: a W at a position corresponding to P36 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and an I at position P90 of SEQ ID NO: 1. In some embodiments, a KOD polymerase variant comprises: a W at position P36 of SEQ ID NO: 1, and an I at position P90 of SEQ ID NO: 1.

### P36W_P90I_R97F

In some embodiments, a KOD polymerase variant comprises: a W at a position corresponding to P36 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position P90 of SEQ ID NO: 1, and a F at position R97 of SEQ ID NO: 1. In some embodiments, a KOD polymerase variant comprises: a W at position P36 of SEQ ID NO: 1, an I at position P90 of SEQ ID NO: 1, and a F at position R97 of SEQ ID NO: 1.

### P36W_P90I_V93I

In some embodiments, a KOD polymerase variant comprises: a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position P90 of SEQ ID NO: 1, and an I at position V93 of SEQ ID NO: 1. In some embodiments, a KOD polymerase variant comprises: a W at position P36 of SEQ ID NO: 1, an I at position P90 of SEQ ID NO: 1, and an I at position V93 of SEQ ID NO: 1.

### P36W_P90I_V93I_R97A

In some embodiments, a KOD polymerase variant comprises: a W at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a W at position P36 of SEQ ID NO: 1, an I at position P90 of SEQ ID NO: 1, an I at position V93 of SEQ ID NO: 1, and an A at position R97 of SEQ ID NO: 1.

### P36W_P90I_V93R_R97A

In some embodiments, a KOD polymerase variant comprises: a W at a position corresponding to P36 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a W at position P36 of SEQ ID NO: 1, an I at position P90 of SEQ ID NO: 1, a R at position V93 of SEQ ID NO: 1, and an A at position R97 of SEQ ID NO: 1.

### P36W_P90I_V93R_R97W

In some embodiments, a KOD polymerase variant comprises: a W at a position corresponding to P36 of SEQ ID NO: 1, an I at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position P90 of SEQ ID NO: 1, a R at position V93 of SEQ ID NO: 1, and a W at position R97 of SEQ ID NO: 1. In some embodiments, a KOD polymerase variant comprises: a W at position P36 of SEQ ID NO: 1, an I at position P90 of SEQ ID NO: 1, a R at position V93 of SEQ ID NO: 1, and a W at position R97 of SEQ ID NO:1.

### P36W_P90S

In some embodiments, a KOD polymerase variant comprises: a W at a position corresponding to P36 of SEQ ID NO: 1 and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a S at position P90 of SEQ ID NO: 1. In some embodiments, a KOD polymerase variant comprises: a W at position P36 of SEQ ID NO: 1, and a S at position P90 of SEQ ID NO: 1.

### P36W_P90W

In some embodiments, a KOD polymerase variant comprises: a W at a position corresponding to P36 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a W at position P90 of SEQ ID NO: 1. In some embodiments, a KOD polymerase variant comprises: a W at position P36 of SEQ ID NO: 1, and a W at position P90 of SEQ ID NO: 1.

### P36W_P90W_R97A

In some embodiments, a KOD polymerase variant comprises: a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, and an A at position R97 of SEQ ID NO: 1. In some embodiments, a KOD polymerase variant comprises: a W at position P36 of SEQ ID NO: 1, a W at position P90 of SEQ ID NO: 1, and an A at position R97 of SEQ ID NO: 1.

### P36W_P90W_R97H

In some embodiments, a KOD polymerase variant comprises: a W at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, and a H at position R97 of SEQ ID NO: 1. In some embodiments, a KOD polymerase variant comprises: a W at position P36 of SEQ ID NO: 1, a W at position P90 of SEQ ID NO: 1, and a H at position R97 of SEQ ID NO: 1.

### P36W_P90W_V93I

In some embodiments, a KOD polymerase variant comprises: a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, and an I at position V93 of SEQ ID NO: 1. In some embodiments, a KOD polymerase variant comprises: a W at position P36 of SEQ ID NO: 1, a W at position P90 of SEQ ID NO: 1, and an I at position V93 of SEQ ID NO: 1.

### P36W_P90W_V93I_R97A

In some embodiments, a KOD polymerase variant comprises: a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a W at position P36 of SEQ ID NO: 1, a W at position P90 of SEQ ID NO: 1, an I at position V93 of SEQ ID NO: 1, and an A at position R97 of SEQ ID NO: 1.

### P36W_P90W_V93I_R97F

In some embodiments, a KOD polymerase variant comprises: a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a W at position P36 of SEQ ID NO: 1, a W at position P90 of SEQ ID NO: 1, an I at position V93 of SEQ ID NO: 1, and a F at position R97 of SEQ ID NO: 1.

### P36W_P90W_V93I_R97H

In some embodiments, a KOD polymerase variant comprises: a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a W at position P36 of SEQ ID NO: 1, a W at position P90 of SEQ ID NO: 1, an I at position V93 of SEQ ID NO: 1, and a H at position R97 of SEQ ID NO: 1.

### P36W_P90W_V93I_R97W

In some embodiments, a KOD polymerase variant comprises: a W at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a W at position P36 of SEQ ID NO: 1, a W at position P90 of SEQ ID NO: 1, an I at position V93 of SEQ ID NO: 1, and a W at position R97 of SEQ ID NO: 1.

### P36W_P90W_V93Q_R97A

In some embodiments, a KOD polymerase variant comprises: a W at a position corresponding to P36 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a W at position P36 of SEQ ID NO: 1, a W at position P90 of SEQ ID NO: 1, a Q at position V93 of SEQ ID NO: 1, and an A at position R97 of SEQ ID NO: 1.

### P36W_P90W_V93R_R97A

In some embodiments, a KOD polymerase variant comprises: a W at a position corresponding to P36 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a W at position P36 of SEQ ID NO: 1, a W at position P90 of SEQ ID NO: 1, a R at position V93 of SEQ ID NO: 1, and an A at position R97 of SEQ ID NO: 1.

### P36W_P90Y

In some embodiments, a KOD polymerase variant comprises: a W at a position corresponding to P36 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a Y at position P90 of SEQ ID NO: 1. In some embodiments, a KOD polymerase variant comprises: a W at position P36 of SEQ ID NO: 1, and a Y at position P90 of SEQ ID NO: 1.

### P36W_P90Y_V93I

In some embodiments, a KOD polymerase variant comprises: a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, and an I at position V93 of SEQ ID NO: 1. In some embodiments, a KOD polymerase variant comprises: a W at position P36 of SEQ ID NO: 1, a Y at position P90 of SEQ ID NO: 1, and an I at position V93 of SEQ ID NO: 1.

### P36W_P90Y_V93I_R97A

In some embodiments, a KOD polymerase variant comprises: a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a W at position P36 of SEQ ID NO: 1, a Y at position P90 of SEQ ID NO: 1, an I at position V93 of SEQ ID NO: 1, and an A at position R97 of SEQ ID NO: 1.

### P36W_P90Y_V93I_R97F

In some embodiments, a KOD polymerase variant comprises: a W at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a W at position P36 of SEQ ID NO: 1, a Y at position P90 of SEQ ID NO: 1, an I at position V93 of SEQ ID NO: 1, and a F at position R97 of SEQ ID NO: 1.

### P36W_P90Y_V93I_R97H

In some embodiments, a KOD polymerase variant comprises: a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a W at position P36 of SEQ ID NO: 1, a Y at position P90 of SEQ ID NO: 1, an I at position V93 of SEQ ID NO: 1, and a H at position R97 of SEQ ID NO: 1.

### P36W_P90Y_V93K

In some embodiments, a KOD polymerase variant comprises: a W at a position corresponding to P36 of SEQ ID NO: 1, a Y at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, and a K at position V93 of SEQ ID NO: 1. In some embodiments, a KOD polymerase variant comprises: a W at position P36 of SEQ ID NO: 1, a Y at position P90 of SEQ ID NO: 1, and a K at position V93 of SEQ ID NO: 1.

### P36W_P90Y_V93R

In some embodiments, a KOD polymerase variant comprises: a W at a position corresponding to P36 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, and a R at position V93 of SEQ ID NO: 1. In some embodiments, a KOD polymerase variant comprises: a W at position P36 of SEQ ID NO: 1, a Y at position P90 of SEQ ID NO: 1, and a R at position V93 of SEQ ID NO: 1.

### P36W_P90Y_V93R_R97A

In some embodiments, a KOD polymerase variant comprises: a W at a position corresponding to P36 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a W at position P36 of SEQ ID NO: 1, a Y at position P90 of SEQ ID NO: 1, a R at position V93 of SEQ ID NO: 1, and an A at position R97 of SEQ ID NO: 1.

### P36W_R97A

In some embodiments, a KOD polymerase variant comprises: a W at a position corresponding to P36 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and an A at position R97 of SEQ ID NO: 1. In some embodiments, a KOD polymerase variant comprises: a W at position P36 of SEQ ID NO: 1, and an A at position R97 of SEQ ID NO: 1.

### P36W_R97F

In some embodiments, a KOD polymerase variant comprises: a W at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a F at position R97 of SEQ ID NO: 1. In some embodiments, a KOD polymerase variant comprises: a W at position P36 of SEQ ID NO: 1, and a F at position R97 of SEQ ID NO: 1.

### P36W_R97H

In some embodiments, a KOD polymerase variant comprises: a W at a position corresponding to P36 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a H at position R97 of SEQ ID NO: 1. In some embodiments, a KOD polymerase variant comprises: a W at position P36 of SEQ ID NO: 1, and a H at position R97 of SEQ ID NO: 1.

### P36W_R97W

In some embodiments, a KOD polymerase variant comprises: a W at a position corresponding to P36 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a W at position R97 of SEQ ID NO: 1. In some embodiments, a KOD polymerase variant comprises: a W at position P36 of SEQ ID NO: 1, and a W at position R97 of SEQ ID NO: 1.

### P36W_V93I

In some embodiments, a KOD polymerase variant comprises: a W at a position corresponding to P36 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and an I at position V93 of SEQ ID NO: 1. In some embodiments, a KOD polymerase variant comprises: a W at position P36 of SEQ ID NO: 1, and an I at position V93 of SEQ ID NO: 1.

### P36W_V93I_R97A

In some embodiments, a KOD polymerase variant comprises: a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position V93 of SEQ ID NO: 1, and an A at position R97 of SEQ ID NO: 1. In some embodiments, a KOD polymerase variant comprises: a W at position P36 of SEQ ID NO: 1, an I at position V93 of SEQ ID NO: 1, and an A at position R97 of SEQ ID NO: 1.

### P36W_V93I_R97F

In some embodiments, a KOD polymerase variant comprises: a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position V93 of SEQ ID NO: 1, and a F at position R97 of SEQ ID NO: 1. In some embodiments, a KOD polymerase variant comprises: a W at position P36 of SEQ ID NO: 1, an I at position V93 of SEQ ID NO: 1, and a F at position R97 of SEQ ID NO: 1.

### P36W_V93I_R97H

In some embodiments, a KOD polymerase variant comprises: a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position V93 of SEQ ID NO: 1, and a H at position R97 of SEQ ID NO: 1. In some embodiments, a KOD polymerase variant comprises: a W at position P36 of SEQ ID NO: 1, an I at position V93 of SEQ ID NO: 1, and a H at position R97 of SEQ ID NO: 1.

### P36W_V93I_R97W

In some embodiments, a KOD polymerase variant comprises: a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, an I at position V93 of SEQ ID NO: 1, and a W at position R97 of SEQ ID NO: 1. In some embodiments, a KOD polymerase variant comprises: a W at position P36 of SEQ ID NO: 1, an I at position V93 of SEQ ID NO: 1, and a W at position R97 of SEQ ID NO: 1.

### P36W_V93Q

In some embodiments, a KOD polymerase variant comprises: a W at a position corresponding to P36 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a Q at position V93 of SEQ ID NO: 1. In some embodiments, a KOD polymerase variant comprises: a W at position P36 of SEQ ID NO: 1, and a Q at position V93 of SEQ ID NO: 1.

### P36W_V93R

In some embodiments, a KOD polymerase variant comprises: a W at a position corresponding to P36 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, and a R at position V93 of SEQ ID NO: 1. In some embodiments, a KOD polymerase variant comprises: a W at position P36 of SEQ ID NO: 1, and a R at position V93 of SEQ ID NO: 1.

### P36W_V93R_R97A

In some embodiments, a KOD polymerase variant comprises: a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a R at position V93 of SEQ ID NO: 1, and an A at position R97 of SEQ ID NO: 1. In some embodiments, a KOD polymerase variant comprises: a W at position P36 of SEQ ID NO: 1, a R at position V93 of SEQ ID NO: 1, and an A at position R97 of SEQ ID NO: 1.

### P36W_V93R_R97F

In some embodiments, a KOD polymerase variant comprises: a W at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a R at position V93 of SEQ ID NO: 1, and a F at position R97 of SEQ ID NO: 1. In some embodiments, a KOD polymerase variant comprises: a W at position P36 of SEQ ID NO: 1, a R at position V93 of SEQ ID NO: 1, and a F at position R97 of SEQ ID NO: 1.

### P36W_V93R_R97H

In some embodiments, a KOD polymerase variant comprises: a W at a position corresponding to P36 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P36 of SEQ ID NO: 1, a R at position V93 of SEQ ID NO: 1, and a H at position R97 of SEQ ID NO: 1. In some embodiments, a KOD polymerase variant comprises: a W at position P36 of SEQ ID NO: 1, a R at position V93 of SEQ ID NO: 1, and a H at position R97 of SEQ ID NO: 1.

### P90F

In some embodiments, a KOD polymerase variant comprises: a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1. In some embodiments, a KOD polymerase variant comprises: a F at position P90 of SEQ ID NO: 1.

### P90F_R97A

In some embodiments, a KOD polymerase variant comprises: a F at a position corresponding to P90 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and an A at position R97 of SEQ ID NO: 1. In some embodiments, a KOD polymerase variant comprises: a F at position P90 of SEQ ID NO: 1, and an A at position R97 of SEQ ID NO: 1.

### P90F_R97F

In some embodiments, a KOD polymerase variant comprises: a F at a position corresponding to P90 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a F at position R97 of SEQ ID NO: 1. In some embodiments, a KOD polymerase variant comprises: a F at position P90 of SEQ ID NO: 1, and a F at position R97 of SEQ ID NO: 1.

### P90F_R97H

In some embodiments, a KOD polymerase variant comprises: a F at a position corresponding to P90 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a H at position R97 of SEQ ID NO: 1. In some embodiments, a KOD polymerase variant comprises: a F at position P90 of SEQ ID NO: 1, and a H at position R97 of SEQ ID NO: 1.

### P90F_R97W

In some embodiments, a KOD polymerase variant comprises: a F at a position corresponding to P90 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a W at position R97 of SEQ ID NO: 1. In some embodiments, a KOD polymerase variant comprises: a F at position P90 of SEQ ID NO: 1, and a W at position R97 of SEQ ID NO: 1.

### P90F_V93I

In some embodiments, a KOD polymerase variant comprises: a F at a position corresponding to P90 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and an I at position V93 of SEQ ID NO: 1. In some embodiments, a KOD polymerase variant comprises: a F at position P90 of SEQ ID NO: 1, and an I at position V93 of SEQ ID NO: 1.

### P90F_V93I_R97F

In some embodiments, a KOD polymerase variant comprises: a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, an I at position V93 of SEQ ID NO: 1, and a F at position R97 of SEQ ID NO: 1. In some embodiments, a KOD polymerase variant comprises: a F at position P90 of SEQ ID NO: 1, an I at position V93 of SEQ ID NO: 1, and a F at position R97 of SEQ ID NO: 1.

### P90F_V93I_R97H

In some embodiments, a KOD polymerase variant comprises: a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, an I at position V93 of SEQ ID NO: 1, and a H at position R97 of SEQ ID NO: 1. In some embodiments, a KOD polymerase variant comprises: a F at position P90 of SEQ ID NO: 1, an I at position V93 of SEQ ID NO: 1, and a H at position R97 of SEQ ID NO: 1.

### P90F_V93I_R97W

In some embodiments, a KOD polymerase variant comprises: a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, an I at position V93 of SEQ ID NO: 1, and a W at position R97 of SEQ ID NO: 1. In some embodiments, a KOD polymerase variant comprises: a F at position P90 of SEQ ID NO: 1, an I at position V93 of SEQ ID NO: 1, and a W at position R97 of SEQ ID NO: 1.

### P90F_V93K

In some embodiments, a KOD polymerase variant comprises: a F at a position corresponding to P90 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a K at position V93 of SEQ ID NO: 1. In some embodiments, a KOD polymerase variant comprises: a F at position P90 of SEQ ID NO: 1, and a K at position V93 of SEQ ID NO: 1.

### P90F_V93K_R97A

In some embodiments, a KOD polymerase variant comprises: a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and an A at position R97 of SEQ ID NO: 1. In some embodiments, a KOD polymerase variant comprises: a F at position P90 of SEQ ID NO: 1, a K at position V93 of SEQ ID NO: 1, and an A at position R97 of SEQ ID NO: 1.

### P90F_V93K_R97F

In some embodiments, a KOD polymerase variant comprises: a F at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a F at position R97 of SEQ ID NO: 1. In some embodiments, a KOD polymerase variant comprises: a F at position P90 of SEQ ID NO: 1, a K at position V93 of SEQ ID NO: 1, and a F at position R97 of SEQ ID NO: 1.

### P90F_V93K_R97H

In some embodiments, a KOD polymerase variant comprises: a F at a position corresponding to P90 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a H at position R97 of SEQ ID NO: 1. In some embodiments, a KOD polymerase variant comprises: a F at position P90 of SEQ ID NO: 1, a K at position V93 of SEQ ID NO: 1, and a H at position R97 of SEQ ID NO: 1.

### P90F_V93Q

In some embodiments, a KOD polymerase variant comprises: a F at a position corresponding to P90 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a Q at position V93 of SEQ ID NO: 1. In some embodiments, a KOD polymerase variant comprises: a F at position P90 of SEQ ID NO: 1, and a Q at position V93 of SEQ ID NO: 1.

### P90F_V93R

In some embodiments, a KOD polymerase variant comprises: a F at a position corresponding to P90 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a R at position V93 of SEQ ID NO: 1. In some embodiments, a KOD polymerase variant comprises: a F at position P90 of SEQ ID NO: 1, and a R at position V93 of SEQ ID NO: 1.

### P90F_V93R_R97A

In some embodiments, a KOD polymerase variant comprises: a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a R at position V93 of SEQ ID NO: 1, and an A at position R97 of SEQ ID NO: 1. In some embodiments, a KOD polymerase variant comprises: a F at position P90 of SEQ ID NO: 1, a R at position V93 of SEQ ID NO: 1, and an A at position R97 of SEQ ID NO: 1.

### P90F_V93R_R97A_R119Q

In some embodiments, a KOD polymerase variant comprises: a F at a position corresponding to P90 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a F at position P90 of SEQ ID NO: 1, a R at position V93 of SEQ ID NO: 1, an A at position R97 of SEQ ID NO: 1, and a Q at position R119 of SEQ ID NO: 1.

### P90F_V93R_R97F

In some embodiments, a KOD polymerase variant comprises: a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a R at position V93 of SEQ ID NO: 1, and a F at position R97 of SEQ ID NO: 1. In some embodiments, a KOD polymerase variant comprises: a F at position P90 of SEQ ID NO: 1, a R at position V93 of SEQ ID NO: 1, and a F at position R97 of SEQ ID NO: 1.

### P90F_V93R_R97H

In some embodiments, a KOD polymerase variant comprises: a F at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a R at position V93 of SEQ ID NO: 1, and a H at position R97 of SEQ ID NO: 1. In some embodiments, a KOD polymerase variant comprises: a F at position P90 of SEQ ID NO: 1, a R at position V93 of SEQ ID NO: 1, and a H at position R97 of SEQ ID NO: 1.

### P90I

In some embodiments, a KOD polymerase variant comprises: an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1. In some embodiments, a KOD polymerase variant comprises: an I at position P90 of SEQ ID NO: 1.

### P90I_R97A

In some embodiments, a KOD polymerase variant comprises: an I at a position corresponding to P90 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and an A at position R97 of SEQ ID NO: 1. In some embodiments, a KOD polymerase variant comprises: an I at position P90 of SEQ ID NO: 1, and an A at position R97 of SEQ ID NO: 1.

### P90I_R97F

In some embodiments, a KOD polymerase variant comprises: an I at a position corresponding to P90 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a F at position R97 of SEQ ID NO: 1. In some embodiments, a KOD polymerase variant comprises: an I at position P90 of SEQ ID NO: 1, and a F at position R97 of SEQ ID NO: 1.

### P90I_R97H

In some embodiments, a KOD polymerase variant comprises: an I at a position corresponding to P90 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a H at position R97 of SEQ ID NO: 1. In some embodiments, a KOD polymerase variant comprises: an I at position P90 of SEQ ID NO: 1, and a H at position R97 of SEQ ID NO: 1.

### P90I_R97W

In some embodiments, a KOD polymerase variant comprises: an I at a position corresponding to P90 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a W at position R97 of SEQ ID NO: 1. In some embodiments, a KOD polymerase variant comprises: an I at position P90 of SEQ ID NO: 1, and a W at position R97 of SEQ ID NO: 1.

### P90I_V93I

In some embodiments, a KOD polymerase variant comprises: an I at a position corresponding to P90 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and an I at position V93 of SEQ ID NO: 1. In some embodiments, a KOD polymerase variant comprises: an I at position P90 of SEQ ID NO: 1, and an I at position V93 of SEQ ID NO: 1.

### P90I_V93I_R97A

In some embodiments, a KOD polymerase variant comprises: an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, an I at position V93 of SEQ ID NO: 1, and an A at position R97 of SEQ ID NO: 1. In some embodiments, a KOD polymerase variant comprises: an I at position P90 of SEQ ID NO: 1, an I at position V93 of SEQ ID NO: 1, and an A at position R97 of SEQ ID NO: 1.

### P90I_V93I_R97F

In some embodiments, a KOD polymerase variant comprises: an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, an I at position V93 of SEQ ID NO: 1, and a F at position R97 of SEQ ID NO: 1. In some embodiments, a KOD polymerase variant comprises: an I at position P90 of SEQ ID NO: 1, an I at position V93 of SEQ ID NO: 1, and a F at position R97 of SEQ ID NO: 1.

### P90I_V93I_R97H

In some embodiments, a KOD polymerase variant comprises: an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, an I at position V93 of SEQ ID NO: 1, and a H at position R97 of SEQ ID NO: 1. In some embodiments, a KOD polymerase variant comprises: an I at position P90 of SEQ ID NO: 1, an I at position V93 of SEQ ID NO: 1, and a H at position R97 of SEQ ID NO: 1.

### P90I_V93I_R97W

In some embodiments, a KOD polymerase variant comprises: an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, an I at position V93 of SEQ ID NO: 1, and a W at position R97 of SEQ ID NO: 1. In some embodiments, a KOD polymerase variant comprises: an I at position P90 of SEQ ID NO: 1, an I at position V93 of SEQ ID NO: 1, and a W at position R97 of SEQ ID NO: 1.

### P90I_V93K

In some embodiments, a KOD polymerase variant comprises: an I at a position corresponding to P90 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a K at position V93 of SEQ ID NO: 1. In some embodiments, a KOD polymerase variant comprises: an I at position P90 of SEQ ID NO: 1, and a K at position V93 of SEQ ID NO: 1.

### P90I_V93K_R97A

In some embodiments, a KOD polymerase variant comprises: an I at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a K at position V93 of SEQ ID NO: 1, and an A at position R97 of SEQ ID NO: 1. In some embodiments, a KOD polymerase variant comprises: an I at position P90 of SEQ ID NO: 1, a K at position V93 of SEQ ID NO: 1, and an A at position R97 of SEQ ID NO: 1.

### P90I_V93K_R97H

In some embodiments, a KOD polymerase variant comprises: an I at a position corresponding to P90 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a K at position V93 of SEQ ID NO: 1, and a H at position R97 of SEQ ID NO: 1. In some embodiments, a KOD polymerase variant comprises: an I at position P90 of SEQ ID NO: 1, a K at position V93 of SEQ ID NO: 1, and a H at position R97 of SEQ ID NO: 1.

### P90I_V93K_R97P

In some embodiments, a KOD polymerase variant comprises: an I at a position corresponding to P90 of SEQ ID NO: 1, a K at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a K at position V93 of SEQ ID NO: 1, and a P at position R97 of SEQ ID NO: 1. In some embodiments, a KOD polymerase variant comprises: an I at position P90 of SEQ ID NO: 1, a K at position V93 of SEQ ID NO: 1, and a P at position R97 of SEQ ID NO: 1.

### P90I_V93Q

In some embodiments, a KOD polymerase variant comprises: an I at a position corresponding to P90 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a Q at position V93 of SEQ ID NO: 1. In some embodiments, a KOD polymerase variant comprises: an I at position P90 of SEQ ID NO: 1, and a Q at position V93 of SEQ ID NO: 1.

### P90I_V93Q_R97A

In some embodiments, a KOD polymerase variant comprises: an I at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a Q at position V93 of SEQ ID NO: 1, and an A at position R97 of SEQ ID NO: 1. In some embodiments, a KOD polymerase variant comprises: an I at position P90 of SEQ ID NO: 1, a Q at position V93 of SEQ ID NO: 1, and an A at position R97 of SEQ ID NO: 1.

### P90I_V93R

In some embodiments, a KOD polymerase variant comprises: an I at a position corresponding to P90 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, and a R at position V93 of SEQ ID NO: 1. In some embodiments, a KOD polymerase variant comprises: an I at position P90 of SEQ ID NO: 1, and a R at position V93 of SEQ ID NO: 1.

### P90I_V93R_R97A

In some embodiments, a KOD polymerase variant comprises: an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a R at position V93 of SEQ ID NO: 1, and an A at position R97 of SEQ ID NO: 1. In some embodiments, a KOD polymerase variant comprises: an I at position P90 of SEQ ID NO: 1, a R at position V93 of SEQ ID NO: 1, and an A at position R97 of SEQ ID NO: 1.

### P90I_V93R_R97F

In some embodiments, a KOD polymerase variant comprises: an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a R at position V93 of SEQ ID NO: 1, and a F at position R97 of SEQ ID NO: 1. In some embodiments, a KOD polymerase variant comprises: an I at position P90 of SEQ ID NO: 1, a R at position V93 of SEQ ID NO: 1, and a F at position R97 of SEQ ID NO: 1.

### P90I_V93R_R97H

In some embodiments, a KOD polymerase variant comprises: an I at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position P90 of SEQ ID NO: 1, a R at position V93 of SEQ ID NO: 1, and a H at position R97 of SEQ ID NO: 1. In some embodiments, a KOD polymerase variant comprises: an I at position P90 of SEQ ID NO: 1, a R at position V93 of SEQ ID NO: 1, and a H at position R97 of SEQ ID NO: 1.

### P90S_R97H

In some embodiments, a KOD polymerase variant comprises: a S at a position corresponding to P90 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, and a H at position R97 of SEQ ID NO: 1. In some embodiments, a KOD polymerase variant comprises: a S at position P90 of SEQ ID NO: 1, and a H at position R97 of SEQ ID NO: 1.

### P90S_R97W

In some embodiments, a KOD polymerase variant comprises: a S at a position corresponding to P90 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, and a W at position R97 of SEQ ID NO: 1. In some embodiments, a KOD polymerase variant comprises: a S at position P90 of SEQ ID NO: 1, and a W at position R97 of SEQ ID NO: 1.

### P90S_V93I_R97A

In some embodiments, a KOD polymerase variant comprises: a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, an I at position V93 of SEQ ID NO: 1, and an A at position R97 of SEQ ID NO: 1. In some embodiments, a KOD polymerase variant comprises: a S at position P90 of SEQ ID NO: 1, an I at position V93 of SEQ ID NO: 1, and an A at position R97 of SEQ ID NO: 1.

### P90S_V93I_R97F

In some embodiments, a KOD polymerase variant comprises: a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, an I at position V93 of SEQ ID NO: 1, and a F at position R97 of SEQ ID NO: 1. In some embodiments, a KOD polymerase variant comprises: a S at position P90 of SEQ ID NO: 1, an I at position V93 of SEQ ID NO: 1, and a F at position R97 of SEQ ID NO: 1.

### P90S_V93I_R97H

In some embodiments, a KOD polymerase variant comprises: a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, an I at position V93 of SEQ ID NO: 1, and a H at position R97 of SEQ ID NO: 1. In some embodiments, a KOD polymerase variant comprises: a S at position P90 of SEQ ID NO: 1, an I at position V93 of SEQ ID NO: 1, and a H at position R97 of SEQ ID NO: 1.

### P90S_V93I_R97W

In some embodiments, a KOD polymerase variant comprises: a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, an I at position V93 of SEQ ID NO: 1, and a W at position R97 of SEQ ID NO: 1. In some embodiments, a KOD polymerase variant comprises: a S at position P90 of SEQ ID NO: 1, an I at position V93 of SEQ ID NO: 1, and a W at position R97 of SEQ ID NO: 1.

### P90S_V93K_R97F

In some embodiments, a KOD polymerase variant comprises: a S at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a K at position V93 of SEQ ID NO: 1, and a F at position R97 of SEQ ID NO: 1. In some embodiments, a KOD polymerase variant comprises: a S at position P90 of SEQ ID NO: 1, a K at position V93 of SEQ ID NO: 1, and a F at position R97 of SEQ ID NO: 1.

### P90S_V93K_R97W

In some embodiments, a KOD polymerase variant comprises: a S at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a K at position V93 of SEQ ID NO: 1, and a W at position R97 of SEQ ID NO: 1. In some embodiments, a KOD polymerase variant comprises: a S at position P90 of SEQ ID NO: 1, a K at position V93 of SEQ ID NO: 1, and a W at position R97 of SEQ ID NO: 1.

### P90S_V93Q

In some embodiments, a KOD polymerase variant comprises: a S at a position corresponding to P90 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, and a Q at position V93 of SEQ ID NO: 1. In some embodiments, a KOD polymerase variant comprises: a S at position P90 of SEQ ID NO: 1, and a Q at position V93 of SEQ ID NO: 1.

### P90S_V93Q_R97A

In some embodiments, a KOD polymerase variant comprises: a S at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a Q at position V93 of SEQ ID NO: 1, and an A at position R97 of SEQ ID NO: 1. In some embodiments, a KOD polymerase variant comprises: a S at position P90 of SEQ ID NO: 1, a Q at position V93 of SEQ ID NO: 1, and an A at position R97 of SEQ ID NO: 1.

### P90S_V93Q_R97F

In some embodiments, a KOD polymerase variant comprises: a S at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a Q at position V93 of SEQ ID NO: 1, and a F at position R97 of SEQ ID NO: 1. In some embodiments, a KOD polymerase variant comprises: a S at position P90 of SEQ ID NO: 1, a Q at position V93 of SEQ ID NO: 1, and a F at position R97 of SEQ ID NO: 1.

### P90S_V93Q_R97W

In some embodiments, a KOD polymerase variant comprises: a S at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a Q at position V93 of SEQ ID NO: 1, and a W at position R97 of SEQ ID NO: 1. In some embodiments, a KOD polymerase variant comprises: a S at position P90 of SEQ ID NO: 1, a Q at position V93 of SEQ ID NO: 1, and a W at position R97 of SEQ ID NO: 1.

### P90S_V93R

In some embodiments, a KOD polymerase variant comprises: a S at a position corresponding to P90 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, and a R at position V93 of SEQ ID NO: 1. In some embodiments, a KOD polymerase variant comprises: a S at position P90 of SEQ ID NO: 1, and a R at position V93 of SEQ ID NO: 1.

### P90S_V93R_R97A

In some embodiments, a KOD polymerase variant comprises: a S at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a R at position V93 of SEQ ID NO: 1, and an A at position R97 of SEQ ID NO: 1. In some embodiments, a KOD polymerase variant comprises: a S at position P90 of SEQ ID NO: 1, a R at position V93 of SEQ ID NO: 1, and an A at position R97 of SEQ ID NO: 1.

### P90S_V93R_R97H

In some embodiments, a KOD polymerase variant comprises: a S at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a R at position V93 of SEQ ID NO: 1, and a H at position R97 of SEQ ID NO: 1. In some embodiments, a KOD polymerase variant comprises: a S at position P90 of SEQ ID NO: 1, a R at position V93 of SEQ ID NO: 1, and a H at position R97 of SEQ ID NO: 1.

### P90S_V93R_R97W

In some embodiments, a KOD polymerase variant comprises: a S at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a S at position P90 of SEQ ID NO: 1, a R at position V93 of SEQ ID NO: 1, and a W at position R97 of SEQ ID NO: 1. In some embodiments, a KOD polymerase variant comprises: a S at position P90 of SEQ ID NO: 1, a R at position V93 of SEQ ID NO: 1, and a W at position R97 of SEQ ID NO: 1.

### P90W

In some embodiments, a KOD polymerase variant comprises: a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1. In some embodiments, a KOD polymerase variant comprises: a W at position P90 of SEQ ID NO: 1.

### P90W_R97A

In some embodiments, a KOD polymerase variant comprises: a W at a position corresponding to P90 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and an A at position R97 of SEQ ID NO: 1. In some embodiments, a KOD polymerase variant comprises: a W at position P90 of SEQ ID NO: 1, and an A at position R97 of SEQ ID NO: 1.

### P90W_R97F

In some embodiments, a KOD polymerase variant comprises: a W at a position corresponding to P90 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a F at position R97 of SEQ ID NO: 1. In some embodiments, a KOD polymerase variant comprises: a W at position P90 of SEQ ID NO: 1, and a F at position R97 of SEQ ID NO: 1.

### P90W_R97H

In some embodiments, a KOD polymerase variant comprises: a W at a position corresponding to P90 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a H at position R97 of SEQ ID NO: 1. In some embodiments, a KOD polymerase variant comprises: a W at position P90 of SEQ ID NO: 1, and a H at position R97 of SEQ ID NO: 1.

### P90W_R97W

In some embodiments, a KOD polymerase variant comprises: a W at a position corresponding to P90 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a W at position R97 of SEQ ID NO: 1. In some embodiments, a KOD polymerase variant comprises: a W at position P90 of SEQ ID NO: 1, and a W at position R97 of SEQ ID NO: 1.

### P90W_V93I

In some embodiments, a KOD polymerase variant comprises: a W at a position corresponding to P90 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and an I at position V93 of SEQ ID NO: 1. In some embodiments, a KOD polymerase variant comprises: a W at position P90 of SEQ ID NO: 1, and an I at position V93 of SEQ ID NO: 1.

### P90W_V93I_R97A

In some embodiments, a KOD polymerase variant comprises: a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, an I at position V93 of SEQ ID NO: 1, and an A at position R97 of SEQ ID NO: 1. In some embodiments, a KOD polymerase variant comprises: a W at position P90 of SEQ ID NO: 1, an I at position V93 of SEQ ID NO: 1, and an A at position R97 of SEQ ID NO: 1.

### P90W_V93I_R97F

In some embodiments, a KOD polymerase variant comprises: a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, an I at position V93 of SEQ ID NO: 1, and a F at position R97 of SEQ ID NO: 1. In some embodiments, a KOD polymerase variant comprises: a W at position P90 of SEQ ID NO: 1, an I at position V93 of SEQ ID NO: 1, and a F at position R97 of SEQ ID NO: 1.

### P90W_V93I_R97H

In some embodiments, a KOD polymerase variant comprises: a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, an I at position V93 of SEQ ID NO: 1, and a H at position R97 of SEQ ID NO: 1. In some embodiments, a KOD polymerase variant comprises: a W at position P90 of SEQ ID NO: 1, an I at position V93 of SEQ ID NO: 1, and a H at position R97 of SEQ ID NO: 1.

### P90W_V93I_R97W

In some embodiments, a KOD polymerase variant comprises: a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, an I at position V93 of SEQ ID NO: 1, and a W at position R97 of SEQ ID NO: 1. In some embodiments, a KOD polymerase variant comprises: a W at position P90 of SEQ ID NO: 1, an I at position V93 of SEQ ID NO: 1, and a W at position R97 of SEQ ID NO: 1.

### P90W_V93K

In some embodiments, a KOD polymerase variant comprises: a W at a position corresponding to P90 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a K at position V93 of SEQ ID NO: 1. In some embodiments, a KOD polymerase variant comprises: a W at position P90 of SEQ ID NO: 1, and a K at position V93 of SEQ ID NO: 1.

### P90W_V93K_R97F

In some embodiments, a KOD polymerase variant comprises: a W at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a K at position V93 of SEQ ID NO: 1, and a F at position R97 of SEQ ID NO: 1. In some embodiments, a KOD polymerase variant comprises: a W at position P90 of SEQ ID NO: 1, a K at position V93 of SEQ ID NO: 1, and a F at position R97 of SEQ ID NO: 1.

### P90W_V93K_R97H

In some embodiments, a KOD polymerase variant comprises: a W at a position corresponding to P90 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a K at position V93 of SEQ ID NO: 1, and a H at position R97 of SEQ ID NO: 1. In some embodiments, a KOD polymerase variant comprises: a W at position P90 of SEQ ID NO: 1, a K at position V93 of SEQ ID NO: 1, and a H at position R97 of SEQ ID NO: 1.

### P90W_V93Q

In some embodiments, a KOD polymerase variant comprises: a W at a position corresponding to P90 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a Q at position V93 of SEQ ID NO: 1. In some embodiments, a KOD polymerase variant comprises: a W at position P90 of SEQ ID NO: 1, and a Q at position V93 of SEQ ID NO: 1.

### P90W_V93Q_R97A

In some embodiments, a KOD polymerase variant comprises: a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a Q at position V93 of SEQ ID NO: 1, and an A at position R97 of SEQ ID NO: 1. In some embodiments, a KOD polymerase variant comprises: a W at position P90 of SEQ ID NO: 1, a Q at position V93 of SEQ ID NO: 1, and an A at position R97 of SEQ ID NO: 1.

### P90W_V93Q_R97H

In some embodiments, a KOD polymerase variant comprises: a W at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a Q at position V93 of SEQ ID NO: 1, and a H at position R97 of SEQ ID NO: 1. In some embodiments, a KOD polymerase variant comprises: a W at position P90 of SEQ ID NO: 1, a Q at position V93 of SEQ ID NO: 1, and a H at position R97 of SEQ ID NO: 1.

### P90W_V93R

In some embodiments, a KOD polymerase variant comprises: a W at a position corresponding to P90 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, and a R at position V93 of SEQ ID NO: 1. In some embodiments, a KOD polymerase variant comprises: a W at position P90 of SEQ ID NO: 1, and a R at position V93 of SEQ ID NO: 1.

### P90W_V93R_R97A

In some embodiments, a KOD polymerase variant comprises: a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a R at position V93 of SEQ ID NO: 1, and an A at position R97 of SEQ ID NO: 1. In some embodiments, a KOD polymerase variant comprises: a W at position P90 of SEQ ID NO: 1, a R at position V93 of SEQ ID NO: 1, and an A at position R97 of SEQ ID NO: 1.

### P90W_V93R_R97F

In some embodiments, a KOD polymerase variant comprises: a W at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a R at position V93 of SEQ ID NO: 1, and a F at position R97 of SEQ ID NO: 1. In some embodiments, a KOD polymerase variant comprises: a W at position P90 of SEQ ID NO: 1, a R at position V93 of SEQ ID NO: 1, and a F at position R97 of SEQ ID NO: 1.

### P90W_V93R_R97H

In some embodiments, a KOD polymerase variant comprises: a W at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a R at position V93 of SEQ ID NO: 1, and a H at position R97 of SEQ ID NO: 1. In some embodiments, a KOD polymerase variant comprises: a W at position P90 of SEQ ID NO: 1, a R at position V93 of SEQ ID NO: 1, and a H at position R97 of SEQ ID NO: 1.

### P90W_V93R_R97W

In some embodiments, a KOD polymerase variant comprises: a W at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position P90 of SEQ ID NO: 1, a R at position V93 of SEQ ID NO: 1, and a W at position R97 of SEQ ID NO: 1. In some embodiments, a KOD polymerase variant comprises: a W at position P90 of SEQ ID NO: 1, a R at position V93 of SEQ ID NO: 1, and a W at position R97 of SEQ ID NO: 1.

### P90Y

In some embodiments, a KOD polymerase variant comprises: a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1. In some embodiments, a KOD polymerase variant comprises: a Y at position P90 of SEQ ID NO: 1.

### P90Y_R97A

In some embodiments, a KOD polymerase variant comprises: a Y at a position corresponding to P90 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and an A at position R97 of SEQ ID NO: 1. In some embodiments, a KOD polymerase variant comprises: a Y at position P90 of SEQ ID NO: 1, and an A at position R97 of SEQ ID NO: 1.

### P90Y_R97F

In some embodiments, a KOD polymerase variant comprises: a Y at a position corresponding to P90 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a F at position R97 of SEQ ID NO: 1. In some embodiments, a KOD polymerase variant comprises: a Y at position P90 of SEQ ID NO: 1, and a F at position R97 of SEQ ID NO: 1.

### P90Y_R97H

In some embodiments, a KOD polymerase variant comprises: a Y at a position corresponding to P90 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a H at position R97 of SEQ ID NO: 1. In some embodiments, a KOD polymerase variant comprises: a Y at position P90 of SEQ ID NO: 1, and a H at position R97 of SEQ ID NO: 1.

### P90Y_R97W

In some embodiments, a KOD polymerase variant comprises: a Y at a position corresponding to P90 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a W at position R97 of SEQ ID NO: 1. In some embodiments, a KOD polymerase variant comprises: a Y at position P90 of SEQ ID NO: 1, and a W at position R97 of SEQ ID NO: 1.

### P90Y_V93I

In some embodiments, a KOD polymerase variant comprises: a Y at a position corresponding to P90 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and an I at position V93 of SEQ ID NO: 1. In some embodiments, a KOD polymerase variant comprises: a Y at position P90 of SEQ ID NO: 1, and an I at position V93 of SEQ ID NO: 1.

### P90Y_V93I_R97A

In some embodiments, a KOD polymerase variant comprises: a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, an I at position V93 of SEQ ID NO: 1, and an A at position R97 of SEQ ID NO: 1. In some embodiments, a KOD polymerase variant comprises: a Y at position P90 of SEQ ID NO: 1, an I at position V93 of SEQ ID NO: 1, and an A at position R97 of SEQ ID NO: 1.

### P90Y_V93I_R97F

In some embodiments, a KOD polymerase variant comprises: a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, an I at position V93 of SEQ ID NO: 1, and a F at position R97 of SEQ ID NO: 1. In some embodiments, a KOD polymerase variant comprises: a Y at position P90 of SEQ ID NO: 1, an I at position V93 of SEQ ID NO: 1, and a F at position R97 of SEQ ID NO: 1.

### P90Y_V93I_R97H

In some embodiments, a KOD polymerase variant comprises: a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, an I at position V93 of SEQ ID NO: 1, and a H at position R97 of SEQ ID NO: 1. In some embodiments, a KOD polymerase variant comprises: a Y at position P90 of SEQ ID NO: 1, an I at position V93 of SEQ ID NO: 1, and a H at position R97 of SEQ ID NO: 1.

### P90Y_V93I_R97W

In some embodiments, a KOD polymerase variant comprises: a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, an I at position V93 of SEQ ID NO: 1, and a W at position R97 of SEQ ID NO: 1. In some embodiments, a KOD polymerase variant comprises: a Y at position P90 of SEQ ID NO: 1, an I at position V93 of SEQ ID NO: 1, and a W at position R97 of SEQ ID NO: 1.

### P90Y_V93K

In some embodiments, a KOD polymerase variant comprises: a Y at a position corresponding to P90 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a K at position V93 of SEQ ID NO: 1. In some embodiments, a KOD polymerase variant comprises: a Y at position P90 of SEQ ID NO: 1, and a K at position V93 of SEQ ID NO: 1.

### P90Y_V93Q

In some embodiments, a KOD polymerase variant comprises: a Y at a position corresponding to P90 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a Q at position V93 of SEQ ID NO: 1. In some embodiments, a KOD polymerase variant comprises: a Y at position P90 of SEQ ID NO: 1, and a Q at position V93 of SEQ ID NO: 1.

### P90Y_V93Q_R97A

In some embodiments, a KOD polymerase variant comprises: a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, a Q at position V93 of SEQ ID NO: 1, and an A at position R97 of SEQ ID NO: 1. In some embodiments, a KOD polymerase variant comprises: a Y at position P90 of SEQ ID NO: 1, a Q at position V93 of SEQ ID NO: 1, and an A at position R97 of SEQ ID NO: 1.

### P90Y_V93Q_R97F

In some embodiments, a KOD polymerase variant comprises: a Y at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, a Q at position V93 of SEQ ID NO: 1, and a F at position R97 of SEQ ID NO: 1. In some embodiments, a KOD polymerase variant comprises: a Y at position P90 of SEQ ID NO: 1, a Q at position V93 of SEQ ID NO: 1, and a F at position R97 of SEQ ID NO: 1.

### P90Y_V93Q_R97W

In some embodiments, a KOD polymerase variant comprises: a Y at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, a Q at position V93 of SEQ ID NO: 1, and a W at position R97 of SEQ ID NO: 1. In some embodiments, a KOD polymerase variant comprises: a Y at position P90 of SEQ ID NO: 1, a Q at position V93 of SEQ ID NO: 1, and a W at position R97 of SEQ ID NO: 1.

### P90Y_V93R

In some embodiments, a KOD polymerase variant comprises: a Y at a position corresponding to P90 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, and a R at position V93 of SEQ ID NO: 1. In some embodiments, a KOD polymerase variant comprises: a Y at position P90 of SEQ ID NO: 1, and a R at position V93 of SEQ ID NO: 1.

### P90Y_V93R_R97F

In some embodiments, a KOD polymerase variant comprises: a Y at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Y at position P90 of SEQ ID NO: 1, a R at position V93 of SEQ ID NO: 1, and a F at position R97 of SEQ ID NO: 1. In some embodiments, a KOD polymerase variant comprises: a Y at position P90 of SEQ ID NO: 1, a R at position V93 of SEQ ID NO: 1, and a F at position R97 of SEQ ID NO: 1.

### R97A

In some embodiments, a KOD polymerase variant comprises: an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position R97 of SEQ ID NO: 1. In some embodiments, a KOD polymerase variant comprises: an A at position R97 of SEQ ID NO: 1.

### R97A_R119M

In some embodiments, a KOD polymerase variant comprises: an A at a position corresponding to R97 of SEQ ID NO: 1 and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an A at position R97 of SEQ ID NO: 1, and a M at position R119 of SEQ ID NO: 1. In some embodiments, a KOD polymerase variant comprises: an A at position R97 of SEQ ID NO: 1, and a M at position R119 of SEQ ID NO: 1.

### R97F

In some embodiments, a KOD polymerase variant comprises: a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1. In some embodiments, a KOD polymerase variant comprises: a F at position R97 of SEQ ID NO: 1.

### R97W

In some embodiments, a KOD polymerase variant comprises: a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position R97 of SEQ ID NO: 1. In some embodiments, a KOD polymerase variant comprises: a W at position R97 of SEQ ID NO: 1.

### R119Q

In some embodiments, a KOD polymerase variant comprises: a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position R119 of SEQ ID NO: 1. In some embodiments, a KOD polymerase variant comprises: a Q at position R119 of SEQ ID NO: 1.

### V93I

In some embodiments, a KOD polymerase variant comprises: an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1. In some embodiments, a KOD polymerase variant comprises: an I at position V93 of SEQ ID NO: 1.

### V93I_R97A

In some embodiments, a KOD polymerase variant comprises: an I at a position corresponding to V93 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and an A at position R97 of SEQ ID NO: 1. In some embodiments, a KOD polymerase variant comprises: an I at position V93 of SEQ ID NO: 1, and an A at position R97 of SEQ ID NO: 1.

### V93I_R97A_R119M

In some embodiments, a KOD polymerase variant comprises: an I at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, an A at position R97 of SEQ ID NO: 1, and a M at position R119 of SEQ ID NO: 1. In some embodiments, a KOD polymerase variant comprises: an I at position V93 of SEQ ID NO: 1, an A at position R97 of SEQ ID NO: 1, and a M at position R119 of SEQ ID NO: 1.

### V93I_R97A_R119Q

In some embodiments, a KOD polymerase variant comprises: an I at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, an A at position R97 of SEQ ID NO: 1, and a Q at position R119 of SEQ ID NO: 1. In some embodiments, a KOD polymerase variant comprises: an I at position V93 of SEQ ID NO: 1, an A at position R97 of SEQ ID NO: 1, and a Q at position R119 of SEQ ID NO: 1.

### V93I_R97F

In some embodiments, a KOD polymerase variant comprises: an I at a position corresponding to V93 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and a F at position R97 of SEQ ID NO: 1. In some embodiments, a KOD polymerase variant comprises: an I at position V93 of SEQ ID NO: 1, and a F at position R97 of SEQ ID NO: 1.

### V93I_R97F_R119Q

In some embodiments, a KOD polymerase variant comprises: an I at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, a F at position R97 of SEQ ID NO: 1, and a Q at position R119 of SEQ ID NO: 1. In some embodiments, a KOD polymerase variant comprises: an I at position V93 of SEQ ID NO: 1, a F at position R97 of SEQ ID NO: 1, and a Q at position R119 of SEQ ID NO: 1.

### V93I_R97H

In some embodiments, a KOD polymerase variant comprises: an I at a position corresponding to V93 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and a H at position R97 of SEQ ID NO: 1. In some embodiments, a KOD polymerase variant comprises: an I at position V93 of SEQ ID NO: 1, and a H at position R97 of SEQ ID NO: 1.

### V93I_R97W

In some embodiments, a KOD polymerase variant comprises: an I at a position corresponding to V93 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and a W at position R97 of SEQ ID NO: 1. In some embodiments, a KOD polymerase variant comprises: an I at position V93 of SEQ ID NO: 1, and a W at position R97 of SEQ ID NO: 1.

### V93I_R119Q

In some embodiments, a KOD polymerase variant comprises: an I at a position corresponding to V93 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and a Q at position R119 of SEQ ID NO: 1. In some embodiments, a KOD polymerase variant comprises: an I at position V93 of SEQ ID NO: 1, and a Q at position R119 of SEQ ID NO: 1.

### V93I_R119Y

In some embodiments, a KOD polymerase variant comprises: an I at a position corresponding to V93 of SEQ ID NO: 1 and a Y at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and an I at position V93 of SEQ ID NO: 1, and a Y at position R119 of SEQ ID NO: 1. In some embodiments, a KOD polymerase variant comprises: an I at position V93 of SEQ ID NO: 1, and a Y at position R119 of SEQ ID NO: 1.

### V93K

In some embodiments, a KOD polymerase variant comprises: a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1. In some embodiments, a KOD polymerase variant comprises: a K at position V93 of SEQ ID NO: 1.

### V93K_R97A

In some embodiments, a KOD polymerase variant comprises: a K at a position corresponding to V93 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and an A at position R97 of SEQ ID NO: 1. In some embodiments, a KOD polymerase variant comprises: a K at position V93 of SEQ ID NO: 1, and an A at position R97 of SEQ ID NO: 1.

### V93K_R97A_R119M

In some embodiments, a KOD polymerase variant comprises: a K at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, an A at position R97 of SEQ ID NO: 1, and a M at position R119 of SEQ ID NO: 1. In some embodiments, a KOD polymerase variant comprises: a K at position V93 of SEQ ID NO: 1, an A at position R97 of SEQ ID NO: 1, and a M at position R119 of SEQ ID NO: 1.

### V93K_R97F

In some embodiments, a KOD polymerase variant comprises: a K at a position corresponding to V93 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a F at position R97 of SEQ ID NO: 1. In some embodiments, a KOD polymerase variant comprises: a K at position V93 of SEQ ID NO: 1, and a F at position R97 of SEQ ID NO: 1.

### V93K_R97F_R119Q

In some embodiments, a KOD polymerase variant comprises: a K at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, and a Q at position R119 of SEQ ID NO: 1. In some embodiments, a KOD polymerase variant comprises: a K at position V93 of SEQ ID NO: 1, a F at position R97 of SEQ ID NO: 1, and a Q at position R119 of SEQ ID NO: 1.

### V93K_R97H

In some embodiments, a KOD polymerase variant comprises: a K at a position corresponding to V93 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a H at position R97 of SEQ ID NO: 1. In some embodiments, a KOD polymerase variant comprises: a K at position V93 of SEQ ID NO: 1, and a H at position R97 of SEQ ID NO: 1.

### V93K_R97W

In some embodiments, a KOD polymerase variant comprises: a K at a position corresponding to V93 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a W at position R97 of SEQ ID NO: 1. In some embodiments, a KOD polymerase variant comprises: a K at position V93 of SEQ ID NO: 1, and a W at position R97 of SEQ ID NO: 1.

### V93K_R119M

In some embodiments, a KOD polymerase variant comprises: a K at a position corresponding to V93 of SEQ ID NO: 1 and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a M at position R119 of SEQ ID NO: 1. In some embodiments, a KOD polymerase variant comprises: a K at position V93 of SEQ ID NO: 1, and a M at position R119 of SEQ ID NO: 1.

### V93K_R119Q

In some embodiments, a KOD polymerase variant comprises: a K at a position corresponding to V93 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a Q at position R119 of SEQ ID NO: 1. In some embodiments, a KOD polymerase variant comprises: a K at position V93 of SEQ ID NO: 1, and a Q at position R119 of SEQ ID NO: 1.

### V93Q

In some embodiments, a KOD polymerase variant comprises: a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position V93 of SEQ ID NO: 1. In some embodiments, a KOD polymerase variant comprises: a Q at position V93 of SEQ ID NO: 1.

### V93Q_R97A

In some embodiments, a KOD polymerase variant comprises: a Q at a position corresponding to V93 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position V93 of SEQ ID NO: 1, and an A at position R97 of SEQ ID NO: 1. In some embodiments, a KOD polymerase variant comprises: a Q at position V93 of SEQ ID NO: 1, and an A at position R97 of SEQ ID NO: 1.

### V93Q_R97H

In some embodiments, a KOD polymerase variant comprises: a Q at a position corresponding to V93 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position V93 of SEQ ID NO: 1, and a H at position R97 of SEQ ID NO: 1. In some embodiments, a KOD polymerase variant comprises: a Q at position V93 of SEQ ID NO: 1, and a H at position R97 of SEQ ID NO: 1.

### V93Q_R97P

In some embodiments, a KOD polymerase variant comprises: a Q at a position corresponding to V93 of SEQ ID NO: 1 and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position V93 of SEQ ID NO: 1, and a P at position R97 of SEQ ID NO: 1. In some embodiments, a KOD polymerase variant comprises: a Q at position V93 of SEQ ID NO: 1, and a P at position R97 of SEQ ID NO: 1.

### V93Q_R97W

In some embodiments, a KOD polymerase variant comprises: a Q at a position corresponding to V93 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position V93 of SEQ ID NO: 1, and a W at position R97 of SEQ ID NO: 1. In some embodiments, a KOD polymerase variant comprises: a Q at position V93 of SEQ ID NO: 1, and a W at position R97 of SEQ ID NO: 1.

### V93R

In some embodiments, a KOD polymerase variant comprises: a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1. In some embodiments, a KOD polymerase variant comprises: a R at position V93 of SEQ ID NO: 1.

### V93R_R97A

In some embodiments, a KOD polymerase variant comprises: a R at a position corresponding to V93 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, and an A at position R97 of SEQ ID NO: 1. In some embodiments, a KOD polymerase variant comprises: a R at position V93 of SEQ ID NO: 1, and an A at position R97 of SEQ ID NO: 1.

### V93R_R97A_R119E

In some embodiments, a KOD polymerase variant comprises: a R at a position corresponding to V93 of SEQ ID NO: 1, an A at a position corresponding to R97 of SEQ ID NO: 1, and an E at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, an A at position R97 of SEQ ID NO: 1, and an E at position R119 of SEQ ID NO: 1. In some embodiments, a KOD polymerase variant comprises: a R at position V93 of SEQ ID NO: 1, an A at position R97 of SEQ ID NO: 1, and an E at position R119 of SEQ ID NO: 1.

### V93R_R97A_R119Y

In some embodiments, a KOD polymerase variant comprises: a R at a position corresponding to V93 of SEQ ID NO: 1, an A at a position corresponding to R97 of SEQ ID NO: 1, and a Y at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, an A at position R97 of SEQ ID NO: 1, and a Y at position R119 of SEQ ID NO: 1. In some embodiments, a KOD polymerase variant comprises: a R at position V93 of SEQ ID NO: 1, an A at position R97 of SEQ ID NO: 1, and a Y at position R119 of SEQ ID NO: 1.

### V93R_R97F

In some embodiments, a KOD polymerase variant comprises: a R at a position corresponding to V93 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, and a F at position R97 of SEQ ID NO: 1. In some embodiments, a KOD polymerase variant comprises: a R at position V93 of SEQ ID NO: 1, and a F at position R97 of SEQ ID NO: 1.

### V93R_R97H

In some embodiments, a KOD polymerase variant comprises: a R at a position corresponding to V93 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, and a H at position R97 of SEQ ID NO: 1. In some embodiments, a KOD polymerase variant comprises: a R at position V93 of SEQ ID NO: 1, and a H at position R97 of SEQ ID NO: 1.

### V93R_R97H_R119M

In some embodiments, a KOD polymerase variant comprises: a R at a position corresponding to V93 of SEQ ID NO: 1, a H at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, a H at position R97 of SEQ ID NO: 1, and a M at position R119 of SEQ ID NO: 1. In some embodiments, a KOD polymerase variant comprises: a R at position V93 of SEQ ID NO: 1, a H at position R97 of SEQ ID NO: 1, and a M at position R119 of SEQ ID NO: 1.

### V93R_R97W

In some embodiments, a KOD polymerase variant comprises: a R at a position corresponding to V93 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, and a W at position R97 of SEQ ID NO: 1. In some embodiments, a KOD polymerase variant comprises: a R at position V93 of SEQ ID NO: 1, and a W at position R97 of SEQ ID NO: 1.

### V93R_R119Q

In some embodiments, a KOD polymerase variant comprises: a R at a position corresponding to V93 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a R at position V93 of SEQ ID NO: 1, and a Q at position R119 of SEQ ID NO: 1. In some embodiments, a KOD polymerase variant comprises: a R at position V93 of SEQ ID NO: 1, and a Q at position R119 of SEQ ID NO: 1.

### Y7C_I16F

In some embodiments, a KOD polymerase variant comprises: a C at a position corresponding to Y7 of SEQ ID NO: 1 and a F at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a F at position I16 of SEQ ID NO: 1. In some embodiments, a KOD polymerase variant comprises: a C at position Y7 of SEQ ID NO: 1, and a F at position I16 of SEQ ID NO: 1.

### Y7C_I16F_P36A_P90W_V93I

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1.

### Y7C_I16F_P36A_P90Y_V93I

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, an A at position P36 of SEQ ID NO: 1, a Y at position P90 of SEQ ID NO: 1, and an I at position V93 of SEQ ID NO: 1.

### Y7C_I16F_P36A_P90Y_V93I_R97A

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7C_I16F_P36A_V93I

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an A at position P36 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, an A at position P36 of SEQ ID NO: 1, and an I at position V93 of SEQ ID NO: 1.

### Y7C_I16F_P36A_V93I_R97F

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1.

### Y7C_I16F_P36H_P90Y_V93I

In some embodiments, a KOD polymerase variant comprises: a C at a position corresponding to Y7 of SEQ ID NO: 1, a F at a position corresponding to I16 of SEQ ID NO: 1, a H at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a H at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a H at position P36 of SEQ ID NO: 1, a Y at position P90 of SEQ ID NO: 1, and an I at position V93 of SEQ ID NO: 1.

### Y7C_I16F_P36S_P90F_V93I

In some embodiments, a KOD polymerase variant comprises: a C at a position corresponding to Y7 of SEQ ID NO: 1, a F at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a S at position P36 of SEQ ID NO: 1, a F at position P90 of SEQ ID NO: 1, and an I at position V93 of SEQ ID NO: 1.

### Y7C_I16F_P36S_P90F_V93I_R97A

In some embodiments, a KOD polymerase variant comprises: a C at a position corresponding to Y7 of SEQ ID NO: 1, a F at a position corresponding to I16 of SEQ ID NO: 1, a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1.

### Y7C_I16F_P36W_P90F

In some embodiments, a KOD polymerase variant comprises: a C at a position corresponding to Y7 of SEQ ID NO: 1, a F at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P36 of SEQ ID NO: 1, and a F at position P90 of SEQ ID NO: 1. In some embodiments, a KOD polymerase variant comprises: a C at position Y7 of SEQ ID NO: 1, a F at position I16 of SEQ ID NO: 1, a W at position P36 of SEQ ID NO: 1, and a F at position P90 of SEQ ID NO: 1.

### Y7C_I16F_P36W_P90F_V93I

In some embodiments, a KOD polymerase variant comprises: a C at a position corresponding to Y7 of SEQ ID NO: 1, a F at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a W at position P36 of SEQ ID NO: 1, a F at position P90 of SEQ ID NO: 1, and an I at position V93 of SEQ ID NO: 1.

### Y7C_I16F_P36W_P90F_V93I_R97A

In some embodiments, a KOD polymerase variant comprises: a C at a position corresponding to Y7 of SEQ ID NO: 1, a F at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7C_I16F_P90F

In some embodiments, a KOD polymerase variant comprises: a C at a position corresponding to Y7 of SEQ ID NO: 1, a F at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a F at position P90 of SEQ ID NO: 1. In some embodiments, a KOD polymerase variant comprises: a C at position Y7 of SEQ ID NO: 1, a F at position I16 of SEQ ID NO: 1, and a F at position P90 of SEQ ID NO: 1.

### Y7C_I16F_P90F_R97A

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, and an A at position R97 of SEQ ID NO: 1.

### Y7C_I16F_P90F_R97F

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, and a F at position R97 of SEQ ID NO: 1.

### Y7C_I16F_P90F_V93I

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, and an I at position V93 of SEQ ID NO: 1.

### Y7C_I16F_P90F_V93I_R97A

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1.

### Y7C_I16F_P90F_V93I_R97F

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1.

### Y7C_I16F_P90F_V93I_R97H

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1.

### Y7C_I16F_P90F_V93I_R97W

In some embodiments, a KOD polymerase variant comprises: a C at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, a F at position P90 of SEQ ID NO: 1, an I at position V93 of SEQ ID NO: 1, and a W at position R97 of SEQ ID NO: 1.

### Y7C_I16F_P90I

In some embodiments, a KOD polymerase variant comprises: a C at a position corresponding to Y7 of SEQ ID NO: 1, a F at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and an I at position P90 of SEQ ID NO: 1. In some embodiments, a KOD polymerase variant comprises: a C at position Y7 of SEQ ID NO: 1, a F at position I16 of SEQ ID NO: 1, and an I at position P90 of SEQ ID NO: 1.

### Y7C_I16F_P90I_V93I

In some embodiments, a KOD polymerase variant comprises: a C at a position corresponding to Y7 of SEQ ID NO: 1, a F at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, an I at position P90 of SEQ ID NO: 1, and an I at position V93 of SEQ ID NO: 1.

### Y7C_I16F_P90I_V93I_R97A

In some embodiments, a KOD polymerase variant comprises: a C at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1.

### Y7C_I16F_P90I_V93I_R97F

In some embodiments, a KOD polymerase variant comprises: a C at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1.

### Y7C_I16F_P90I_V93I_R97H

In some embodiments, a KOD polymerase variant comprises: a C at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1.

### Y7C_I16F_P90I_V93R

In some embodiments, a KOD polymerase variant comprises: a C at a position corresponding to Y7 of SEQ ID NO: 1, a F at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position P90 of SEQ ID NO: 1, and a R at position V93 of SEQ ID NO: 1. In some embodiments, a KOD polymerase variant comprises: a C at position Y7 of SEQ ID NO: 1, a F at position I16 of SEQ ID NO: 1, an I at position P90 of SEQ ID NO: 1, and a R at position V93 of SEQ ID NO: 1.

### Y7C_I16F_P90S_R97A

In some embodiments, a KOD polymerase variant comprises: a C at a position corresponding to Y7 of SEQ ID NO: 1, a F at a position corresponding to I16 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a S at position P90 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a S at position P90 of SEQ ID NO: 1, and an A at position R97 of SEQ ID NO: 1.

### Y7C_I16F_P90W

In some embodiments, a KOD polymerase variant comprises: a C at a position corresponding to Y7 of SEQ ID NO: 1, a F at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a W at position P90 of SEQ ID NO: 1. In some embodiments, a KOD polymerase variant comprises: a C at position Y7 of SEQ ID NO: 1, a F at position I16 of SEQ ID NO: 1, and a W at position P90 of SEQ ID NO: 1.

### Y7C_I16F_P90W_R97A

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, and an A at position R97 of SEQ ID NO:1.

### Y7C_I16F_P90W_R97F

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, and a F at position R97 of SEQ ID NO: 1.

### Y7C_I16F_P90W_R97H

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, and a H at position R97 of SEQ ID NO: 1.

### Y7C_I16F_P90W_R97W

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, and a W at position R97 of SEQ ID NO: 1.

### Y7C_I16F_P90W_V93I

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, and an I at position V93 of SEQ ID NO: 1.

### Y7C_I16F_P90W_V93I_R97A

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1.

### Y7C_I16F_P90W_V93I_R97F

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1.

### Y7C_I16F_P90W_V93I_R97H

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1.

### Y7C_I16F_P90W_V93I_R97W

In some embodiments, a KOD polymerase variant comprises: a C at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, a W at position P90 of SEQ ID NO: 1, an I at position V93 of SEQ ID NO: 1, and a W at position R97 of SEQ ID NO: 1.

### Y7C_I16F_P90Y

In some embodiments, a KOD polymerase variant comprises: a C at a position corresponding to Y7 of SEQ ID NO: 1, a F at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a Y at position P90 of SEQ ID NO: 1. In some embodiments, a KOD polymerase variant comprises: a C at position Y7 of SEQ ID NO: 1, a F at position I16 of SEQ ID NO: 1, and a Y at position P90 of SEQ ID NO: 1.

### Y7C_I16F_P90Y_R97A

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, and an A at position R97 of SEQ ID NO: 1.

### Y7C_I16F_P90Y_V93I

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, and an I at position V93 of SEQ ID NO: 1.

### Y7C_I16F_P90Y_V93I_R97A

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1.

### Y7C_I16F_P90Y_V93I_R97F

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1.

### Y7C_I16F_P90Y_V93I_R97H

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1.

### Y7C_I16F_P90Y_V93I_R97W

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1.

### Y7C_I16F_P90Y_V93K

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, and a K at position V93 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, and a K at position V93 of SEQ ID NO: 1.

### Y7C_I16F_P90Y_V93K_R97A

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Y at position P90 of SEQ ID NO: 1, a K at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a Y at position P90 of SEQ ID NO: 1, a K at position V93 of SEQ ID NO: 1, and an A at position R97 of SEQ ID NO: 1.

### Y7C_I16F_R97A

In some embodiments, a KOD polymerase variant comprises: a C at a position corresponding to Y7 of SEQ ID NO: 1, a F at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, and an A at position R97 of SEQ ID NO: 1.

### Y7C_I16F_R97F

In some embodiments, a KOD polymerase variant comprises: a C at a position corresponding to Y7 of SEQ ID NO: 1, a F at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, and a F at position R97 of SEQ ID NO: 1.

### Y7C_I16F_R97W

In some embodiments, a KOD polymerase variant comprises: a C at a position corresponding to Y7 of SEQ ID NO: 1, a F at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, and a W at position R97 of SEQ ID NO: 1.

### Y7C_I16F_V93I

In some embodiments, a KOD polymerase variant comprises: a C at a position corresponding to Y7 of SEQ ID NO: 1, a F at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and an I at position V93 of SEQ ID NO: 1. In some embodiments, a KOD polymerase variant comprises: a C at position Y7 of SEQ ID NO: 1, a F at position I16 of SEQ ID NO: 1, and an I at position V93 of SEQ ID NO: 1.

### Y7C_I16F_V93I_R97A

In some embodiments, a KOD polymerase variant comprises: a C at a position corresponding to Y7 of SEQ ID NO: 1, a F at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, an I at position V93 of SEQ ID NO: 1, and an A at position R97 of SEQ ID NO: 1.

### Y7C_I16F_V93I_R97F

In some embodiments, a KOD polymerase variant comprises: a C at a position corresponding to Y7 of SEQ ID NO: 1, a F at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, an I at position V93 of SEQ ID NO: 1, and a F at position R97 of SEQ ID NO: 1.

### Y7C_I16F_V93I_R97H

In some embodiments, a KOD polymerase variant comprises: a C at a position corresponding to Y7 of SEQ ID NO: 1, a F at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a F at position I16 of SEQ ID NO: 1, an I at position V93 of SEQ ID NO: 1, and a H at position R97 of SEQ ID NO: 1.

### Y7C_I16F_V93I_R97W

In some embodiments, a KOD polymerase variant comprises: a C at a position corresponding to Y7 of SEQ ID NO: 1, a F at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, an I at position V93 of SEQ ID NO: 1, and a W at position R97 of SEQ ID NO: 1.

### Y7C_I16F_V93Q

In some embodiments, a KOD polymerase variant comprises: a C at a position corresponding to Y7 of SEQ ID NO: 1, a F at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a Q at position V93 of SEQ ID NO: 1. In some embodiments, a KOD polymerase variant comprises: a C at position Y7 of SEQ ID NO: 1, a F at position I16 of SEQ ID NO: 1, and a Q at position V93 of SEQ ID NO: 1.

### Y7C_I16F_V93Q_R97A

In some embodiments, a KOD polymerase variant comprises: a C at a position corresponding to Y7 of SEQ ID NO: 1, a F at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a Q at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a Q at position V93 of SEQ ID NO: 1, and an A at position R97 of SEQ ID NO: 1.

### Y7C_I16F_V93R

In some embodiments, a KOD polymerase variant comprises: a C at a position corresponding to Y7 of SEQ ID NO: 1, a F at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, and a R at position V93 of SEQ ID NO: 1. In some embodiments, a KOD polymerase variant comprises: a C at position Y7 of SEQ ID NO: 1, a F at position I16 of SEQ ID NO: 1, and a R at position V93 of SEQ ID NO: 1.

### Y7C_I16F_V93R_R97A

In some embodiments, a KOD polymerase variant comprises: a C at a position corresponding to Y7 of SEQ ID NO: 1, a F at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, a F at position I16 of SEQ ID NO: 1, a R at position V93 of SEQ ID NO: 1, and an A at position R97 of SEQ ID NO: 1.

### Y7C_I16F_V93R_R97A_R119Q

In some embodiments, a KOD polymerase variant comprises: a C at a position corresponding to Y7 of SEQ ID NO: 1, a F at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a C at position Y7 of SEQ ID NO: 1, a F at position I16 of SEQ ID NO: 1, a R at position V93 of SEQ ID NO: 1, an A at position R97 of SEQ ID NO: 1, and a Q at position R119 of SEQ ID NO: 1.

### Y7C_I16F_V93R_R97F

In some embodiments, a KOD polymerase variant comprises: a C at a position corresponding to Y7 of SEQ ID NO: 1, a F at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a R at position V93 of SEQ ID NO: 1, and a F at position R97 of SEQ ID NO: 1. In some embodiments, a KOD polymerase variant comprises: a C at position Y7 of SEQ ID NO: 1, a F at position I16 of SEQ ID NO: 1, a R at position V93 of SEQ ID NO: 1, and a F at position R97 of SEQ ID NO: 1.

### Y7C_I16F_V93R_R97W

In some embodiments, a KOD polymerase variant comprises: a C at a position corresponding to Y7 of SEQ ID NO: 1, a F at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position I16 of SEQ ID NO: 1, a R at position V93 of SEQ ID NO: 1, and a W at position R97 of SEQ ID NO: 1. In some embodiments, a KOD polymerase variant comprises: a C at position Y7 of SEQ ID NO: 1, a F at position I16 of SEQ ID NO: 1, a R at position V93 of SEQ ID NO: 1, and a W at position R97 of SEQ ID NO: 1.

### Y7C_I16L_P36A_P90F_V93I

In some embodiments, a KOD polymerase variant comprises: a C at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1.

### Y7C_I16L_P36A_P90W_V93I

In some embodiments, a KOD polymerase variant comprises: a C at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1.

### Y7C_I16L_P36A_P90W_V93I_R97A

In some embodiments, a KOD polymerase variant comprises: a C at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### Y7C_I16L_P36A_P90Y_R97H

In some embodiments, a KOD polymerase variant comprises: a C at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a Y at position P90 of SEQ ID NO: 1, and a H at position R97 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a Y at position P90 of SEQ ID NO: 1, and a H at position R97 of SEQ ID NO: 1.

### Y7C_I16L_P36A_P90Y_V93I

In some embodiments, a KOD polymerase variant comprises: a C at a position corresponding to Y7 of SEQ ID NO: 1, a L at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1.

### Y7C_I16L_P36A_P90Y_V93Q

In some embodiments, a KOD polymerase variant comprises: a C at a position corresponding to Y7 of SEQ ID NO: 1, a L at a position corresponding to I16 of SEQ ID NO: 1, an A at a position corresponding to P36 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an A at position P36 of SEQ ID NO: 1, a Y at position P90 of SEQ ID NO: 1, and a Q at position V93 of SEQ ID NO: 1. In some embodiments, a KOD polymerase variant comprises: a C at position Y7 of SEQ ID NO: 1, a L at position I16 of SEQ ID NO: 1, an A at position P36 of SEQ ID NO: 1, a Y at position P90 of SEQ ID NO: 1, and a Q at position V93 of SEQ ID NO: 1.

### Y7C_I16L_P36H_P90W_V93I

In some embodiments, a KOD polymerase variant comprises: a C at a position corresponding to Y7 of SEQ ID NO: 1, a L at a position corresponding to I16 of SEQ ID NO: 1, a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a H at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a H at position P36 of SEQ ID NO: 1, a W at position P90 of SEQ ID NO: 1, and an I at position V93 of SEQ ID NO: 1.

### Y7C_I16L_P36H_V93I

In some embodiments, a KOD polymerase variant comprises: a C at a position corresponding to Y7 of SEQ ID NO: 1, a L at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a H at position P36 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a H at position P36 of SEQ ID NO: 1, and an I at position V93 of SEQ ID NO: 1.

### Y7C_I16L_P36S_P90F

In some embodiments, a KOD polymerase variant comprises: a C at a position corresponding to Y7 of SEQ ID NO: 1, a L at a position corresponding to I16 of SEQ ID NO: 1, a S at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a S at position P36 of SEQ ID NO: 1, and a F at position P90 of SEQ ID NO: 1. In some embodiments, a KOD polymerase variant comprises: a C at position Y7 of SEQ ID NO: 1, a L at position I16 of SEQ ID NO: 1, a S at position P36 of SEQ ID NO: 1, and a F at position P90 of SEQ ID NO: 1.

### Y7C_I16L_P36S_P90W

In some embodiments, a KOD polymerase variant comprises: a C at a position corresponding to Y7 of SEQ ID NO: 1, a L at a position corresponding to I16 of SEQ ID NO: 1, a S at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a S at position P36 of SEQ ID NO: 1, and a W at position P90 of SEQ ID NO: 1. In some embodiments, a KOD polymerase variant comprises: a C at position Y7 of SEQ ID NO: 1, a L at position I16 of SEQ ID NO: 1, a S at position P36 of SEQ ID NO: 1, and a W at position P90 of SEQ ID NO: 1.

### Y7C_I16L_P36W_P90F

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, and a F at position P90 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, and a F at position P90 of SEQ ID NO: 1.

### Y7C_I16L_P36W_P90F_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a F at position P90 of SEQ ID NO: 1, and an A at position R97 of SEQ ID NO: 1.

### Y7C_I16L_P36W_P90F_V93I

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1.

### Y7C_I16L_P36W_P90F_V93I_R97F

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### Y7C_I16L_P36W_P90W_V93I

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1.

### Y7C_I16L_P36W_P90W_V93I_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### Y7C_I16L_P36W_P90Y

In some embodiments, a KOD polymerase variant comprises: a C at a position corresponding to Y7 of SEQ ID NO: 1, a F at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, and a Y at position P90 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, and a Y at position P90 of SEQ ID NO: 1.

### Y7C_I16L_P36W_P90Y_V93I

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1.

### Y7C_I16L_P36W_V93I

In some embodiments, a KOD polymerase variant comprises: a C at a position corresponding to Y7 of SEQ ID NO: 1, a L at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P36 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a W at position P36 of SEQ ID NO: 1, and an I at position V93 of SEQ ID NO: 1.

### Y7C_I16L_P90F

In some embodiments, a KOD polymerase variant comprises: a C at a position corresponding to Y7 of SEQ ID NO: 1, a L at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and a F at position P90 of SEQ ID NO: 1. In some embodiments, a KOD polymerase variant comprises: a C at position Y7 of SEQ ID NO: 1, a L at position I16 of SEQ ID NO: 1, and a F at position P90 of SEQ ID NO: 1.

### Y7C_I16L_P90F_V93I

In some embodiments, a KOD polymerase variant comprises: a C at a position corresponding to Y7 of SEQ ID NO: 1, a L at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a F at position P90 of SEQ ID NO: 1, and an I at position V93 of SEQ ID NO: 1.

### Y7C_I16L_P90F_V93I_R97A

In some embodiments, a KOD polymerase variant comprises: a C at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1.

### Y7C_I16L_P90F_V93I_R97W

In some embodiments, a KOD polymerase variant comprises: a C at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a L at position I16 of SEQ ID NO: 1, a F at position P90 of SEQ ID NO: 1, an I at position V93 of SEQ ID NO: 1, and a W at position R97 of SEQ ID NO: 1.

### Y7C_I16L_P90F_V93Q

In some embodiments, a KOD polymerase variant comprises: a C at a position corresponding to Y7 of SEQ ID NO: 1, a L at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a F at position P90 of SEQ ID NO: 1, and a Q at position V93 of SEQ ID NO: 1. In some embodiments, a KOD polymerase variant comprises: a C at position Y7 of SEQ ID NO: 1, a L at position I16 of SEQ ID NO: 1, a F at position P90 of SEQ ID NO: 1, and a Q at position V93 of SEQ ID NO: 1.

### Y7C_I16L_P90F_V93Q_R97F

In some embodiments, a KOD polymerase variant comprises: a C at a position corresponding to Y7 of SEQ ID NO: 1, a L at a position corresponding to I16 of SEQ ID NO: 1, a F at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a F at position P90 of SEQ ID NO: 1, a Q at position V93 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, a F at position P90 of SEQ ID NO: 1, a Q at position V93 of SEQ ID NO: 1, and a F at position R97 of SEQ ID NO: 1.

### Y7C_I16L_P90I

In some embodiments, a KOD polymerase variant comprises: a C at a position corresponding to Y7 of SEQ ID NO: 1, a L at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and an I at position P90 of SEQ ID NO: 1. In some embodiments, a KOD polymerase variant comprises: a C at position Y7 of SEQ ID NO: 1, a L at position I16 of SEQ ID NO: 1, and an I at position P90 of SEQ ID NO: 1.

### Y7C_I16L_P90I_V93I

In some embodiments, a KOD polymerase variant comprises: a C at a position corresponding to Y7 of SEQ ID NO: 1, a L at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an I at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, an I at position P90 of SEQ ID NO: 1, and an I at position V93 of SEQ ID NO: 1.

### Y7C_I16L_P90I_V93I_R97A

In some embodiments, a KOD polymerase variant comprises: a C at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1.

### Y7C_I16L_P90W

In some embodiments, a KOD polymerase variant comprises: a C at a position corresponding to Y7 of SEQ ID NO: 1, a L at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and a W at position P90 of SEQ ID NO: 1. In some embodiments, a KOD polymerase variant comprises: a C at position Y7 of SEQ ID NO: 1, a L at position I16 of SEQ ID NO: 1, and a W at position P90 of SEQ ID NO: 1.

### Y7C_I16L_P90W_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and an A at position R97 of SEQ ID NO: 1.

### Y7C_I16L_P90W_R97F

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and a F at position R97 of SEQ ID NO: 1.

### Y7C_I16L_P90W_R97H

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and a H at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and a H at position R97 of SEQ ID NO: 1.

### Y7C_I16L_P90W_V93I

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and an I at position V93 of SEQ ID NO: 1.

### Y7C_I16L_P90W_V93I_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1.

### Y7C_I16L_P90W_V93I_R97F

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1.

### Y7C_I16L_P90W_V93I_R97H

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1.

### Y7C_I16L_P90W_V93Q

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and a Q at position V93 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and a Q at position V93 of SEQ ID NO: 1.

### Y7C_I16L_P90W_V93Q_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1.

### Y7C_I16L_P90W_V93R

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, and a R at position V93 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, and a R at position V93 of SEQ ID NO: 1.

### Y7C_I16L_P90W_V93R_R97A

In some embodiments, a KOD polymerase variant comprises: a C at a position corresponding to Y7 of SEQ ID NO: 1, a L at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1.

### Y7C_I16L_P90Y

In some embodiments, a KOD polymerase variant comprises: a C at a position corresponding to Y7 of SEQ ID NO: 1, a L at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and a Y at position P90 of SEQ ID NO: 1. In some embodiments, a KOD polymerase variant comprises: a C at position Y7 of SEQ ID NO: 1, a L at position I16 of SEQ ID NO: 1, and a Y at position P90 of SEQ ID NO: 1.

### Y7C_I16L_P90Y_V93I

In some embodiments, a KOD polymerase variant comprises: a C at a position corresponding to Y7 of SEQ ID NO: 1, a F at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a L at position I16 of SEQ ID NO: 1, a Y at position P90 of SEQ ID NO: 1, and an I at position V93 of SEQ ID NO: 1.

### Y7C_I16L_P90Y_V93I_R97A

In some embodiments, a KOD polymerase variant comprises: a C at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1.

### Y7C_I16L_P90Y_V93I_R97F

In some embodiments, a KOD polymerase variant comprises: a C at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1.

### Y7C_I16L_P90Y_V93Q

In some embodiments, a KOD polymerase variant comprises: a C at a position corresponding to Y7 of SEQ ID NO: 1, a L at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a Y at position P90 of SEQ ID NO: 1, and a Q at position V93 of SEQ ID NO: 1. In some embodiments, a KOD polymerase variant comprises: a C at position Y7 of SEQ ID NO: 1, a L at position I16 of SEQ ID NO: 1, a Y at position P90 of SEQ ID NO: 1, and a Q at position V93 of SEQ ID NO: 1.

### Y7C_I16L_V93I_R97F

In some embodiments, a KOD polymerase variant comprises: a C at a position corresponding to Y7 of SEQ ID NO: 1, a L at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a L at position I16 of SEQ ID NO: 1, an I at position V93 of SEQ ID NO: 1, and a F at position R97 of SEQ ID NO: 1.

### Y7C_I16L_V93Q

In some embodiments, a KOD polymerase variant comprises: a C at a position corresponding to Y7 of SEQ ID NO: 1, a L at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and a Q at position V93 of SEQ ID NO: 1. In some embodiments, a KOD polymerase variant comprises: a C at position Y7 of SEQ ID NO: 1, a L at position I16 of SEQ ID NO: 1, and a Q at position V93 of SEQ ID NO: 1.

### Y7C_I16L_V93R

In some embodiments, a KOD polymerase variant comprises: a C at a position corresponding to Y7 of SEQ ID NO: 1, a L at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, and a R at position V93 of SEQ ID NO: 1. In some embodiments, a KOD polymerase variant comprises: a C at position Y7 of SEQ ID NO: 1, a L at position I16 of SEQ ID NO: 1, and a R at position V93 of SEQ ID NO: 1.

### Y7C_I16L_V93R_R97W

In some embodiments, a KOD polymerase variant comprises: a C at a position corresponding to Y7 of SEQ ID NO: 1, a L at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a R at position V93 of SEQ ID NO: 1, and a W at position R97 of SEQ ID NO: 1. In some embodiments, a KOD polymerase variant comprises: a C at position Y7 of SEQ ID NO: 1, a L at position I16 of SEQ ID NO: 1, a R at position V93 of SEQ ID NO: 1, and a W at position R97 of SEQ ID NO: 1.

### Y7C_I16L_V93R_R119Q

In some embodiments, a KOD polymerase variant comprises: a C at a position corresponding to Y7 of SEQ ID NO: 1, a L at a position corresponding to I16 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a L at position I16 of SEQ ID NO: 1, a R at position V93 of SEQ ID NO: 1, and a Q at position R119 of SEQ ID NO: 1. In some embodiments, a KOD polymerase variant comprises: a C at position Y7 of SEQ ID NO: 1, a L at position I16 of SEQ ID NO: 1, a R at position V93 of SEQ ID NO: 1, and a Q at position R119 of SEQ ID NO: 1.

### Y7C_I16M

In some embodiments, a KOD polymerase variant comprises: a C at a position corresponding to Y7 of SEQ ID NO: 1 and a M at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a M at position I16 of SEQ ID NO: 1. In some embodiments, a KOD polymerase variant comprises: a C at position Y7 of SEQ ID NO: 1, and a M at position I16 of SEQ ID NO: 1.

### Y7C_I16M_P36A_P90F

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, and a F at position P90 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, and a F at position P90 of SEQ ID NO: 1.

### Y7C_I16M_P36A_P90F_V93I

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, a F at position P90 of SEQ ID NO: 1, and an I at position V93 of SEQ ID NO: 1.

### Y7C_I16M_P36A_P90W

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, and a W at position P90 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, and a W at position P90 of SEQ ID NO: 1.

### Y7C_I16M_P36A_P90W_V93I

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1.

### Y7C_I16M_P36A_P90W_V93I_R97H

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1.

### Y7C_I16M_P36A_V93R_R97A

In some embodiments, a KOD polymerase variant comprises: a C at a position corresponding to Y7 of SEQ ID NO: 1, a M at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1.

### Y7C_I16M_P36H_V93I_R97H

In some embodiments, a KOD polymerase variant comprises: a C at a position corresponding to Y7 of SEQ ID NO: 1, a M at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a M at position I16 of SEQ ID NO: 1, a H at position P36 of SEQ ID NO: 1, an I at position V93 of SEQ ID NO: 1, and a H at position R97 of SEQ ID NO: 1.

### Y7C_I16M_P36W

In some embodiments, a KOD polymerase variant comprises: a C at a position corresponding to Y7 of SEQ ID NO: 1, a M at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and a W at position P36 of SEQ ID NO: 1. In some embodiments, a KOD polymerase variant comprises: a C at position Y7 of SEQ ID NO: 1, a M at position I16 of SEQ ID NO: 1, and a W at position P36 of SEQ ID NO: 1.

### Y7C_I16M_P36W_P90F

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, and a F at position P90 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, and a F at position P90 of SEQ ID NO: 1.

### Y7C_I16M_P36W_P90F_V93I

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1.

### Y7C_I16M_P36W_P90I_V93K

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, an I at position P90 of SEQ ID NO: 1, and a K at position V93 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, an I at position P90 of SEQ ID NO: 1, and a K at position V93 of SEQ ID NO: 1.

### Y7C_I16M_P36W_P90W

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, and a W at position P90 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, and a W at position P90 of SEQ ID NO: 1.

### Y7C_I16M_P36W_P90W_V93I

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1.

### Y7C_I16M_P36W_P90W_V93I_R97W

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1.

### Y7C_I16M_P36W_P90Y_V93I

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1.

### Y7C_I16M_P36W_V93I

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, and an I at position V93 of SEQ ID NO: 1.

### Y7C_I16M_P36W_V93I_R97A

In some embodiments, a KOD polymerase variant comprises: a C at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1.

### Y7C_I16M_P90F

In some embodiments, a KOD polymerase variant comprises: a C at a position corresponding to Y7 of SEQ ID NO: 1, a M at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and a F at position P90 of SEQ ID NO: 1. In some embodiments, a KOD polymerase variant comprises: a C at position Y7 of SEQ ID NO: 1, a M at position I16 of SEQ ID NO: 1, and a F at position P90 of SEQ ID NO: 1.

### Y7C_I16M_P90F_V93I

In some embodiments, a KOD polymerase variant comprises: a C at a position corresponding to Y7 of SEQ ID NO: 1, a M at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a F at position P90 of SEQ ID NO: 1, and an I at position V93 of SEQ ID NO: 1.

### Y7C_I16M_P90F_V93K_R97A

In some embodiments, a KOD polymerase variant comprises: a C at a position corresponding to Y7 of SEQ ID NO: 1, a M at a position corresponding to I16 of SEQ ID NO: 1, a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a F at position P90 of SEQ ID NO: 1, a K at position V93 of SEQ ID NO: 1, and an A at position R97 of SEQ ID NO: 1. In some embodiments, a KOD polymerase variant comprises: a C at position Y7 of SEQ ID NO: 1, a M at position I16 of SEQ ID NO: 1, a F at position P90 of SEQ ID NO: 1, a K at position V93 of SEQ ID NO: 1, and an A at position R97 of SEQ ID NO: 1.

### Y7C_I16M_P90F_V93Q

In some embodiments, a KOD polymerase variant comprises: a C at a position corresponding to Y7 of SEQ ID NO: 1, a M at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a F at position P90 of SEQ ID NO: 1, and a Q at position V93 of SEQ ID NO: 1. In some embodiments, a KOD polymerase variant comprises: a C at position Y7 of SEQ ID NO: 1, a M at position I16 of SEQ ID NO: 1, a F at position P90 of SEQ ID NO: 1, and a Q at position V93 of SEQ ID NO: 1.

### Y7C_I16M_P90I_V93I

In some embodiments, a KOD polymerase variant comprises: a C at a position corresponding to Y7 of SEQ ID NO: 1, a M at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an I at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, an I at position P90 of SEQ ID NO: 1, and an I at position V93 of SEQ ID NO: 1.

### Y7C_I16M_P90I_V93R

In some embodiments, a KOD polymerase variant comprises: a C at a position corresponding to Y7 of SEQ ID NO: 1, a M at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an I at position P90 of SEQ ID NO: 1, and a R at position V93 of SEQ ID NO: 1. In some embodiments, a KOD polymerase variant comprises: a C at position Y7 of SEQ ID NO: 1, a M at position I16 of SEQ ID NO: 1, an I at position P90 of SEQ ID NO: 1, and a R at position V93 of SEQ ID NO: 1.

### Y7C_I16M_P90W

In some embodiments, a KOD polymerase variant comprises: a C at a position corresponding to Y7 of SEQ ID NO: 1, a M at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and a W at position P90 of SEQ ID NO: 1. In some embodiments, a KOD polymerase variant comprises: a C at position Y7 of SEQ ID NO: 1, a M at position I16 of SEQ ID NO: 1, and a W at position P90 of SEQ ID NO: 1.

### Y7C_I16M_P90W_R97H

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, and a H at position R97 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, and a H at position R97 of SEQ ID NO: 1.

### Y7C_I16M_P90W_V93I

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, and an I at position V93 of SEQ ID NO: 1.

### Y7C_I16M_P90W_V93I_R97A

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1.

### Y7C_I16M_P90W_V93I_R97F

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, an I at position V93 of SEQ ID NO: 1, and a F at position R97 of SEQ ID NO: 1.

### Y7C_I16M_P90W_V93I_R97H

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1.

### Y7C_I16M_P90W_V93Q

In some embodiments, a KOD polymerase variant comprises: a C at a position corresponding to Y7 of SEQ ID NO: 1, a M at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a W at position P90 of SEQ ID NO: 1, and a Q at position V93 of SEQ ID NO: 1. In some embodiments, a KOD polymerase variant comprises: a C at position Y7 of SEQ ID NO: 1, a M at position I16 of SEQ ID NO: 1, a W at position P90 of SEQ ID NO: 1, and a Q at position V93 of SEQ ID NO: 1.

### Y7C_I16M_P90Y

In some embodiments, a KOD polymerase variant comprises: a C at a position corresponding to Y7 of SEQ ID NO: 1, a M at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and a Y at position P90 of SEQ ID NO: 1. In some embodiments, a KOD polymerase variant comprises: a C at position Y7 of SEQ ID NO: 1, a M at position I16 of SEQ ID NO: 1, and a Y at position P90 of SEQ ID NO: 1.

### Y7C_I16M_P90Y_V93I

In some embodiments, a KOD polymerase variant comprises: a C at a position corresponding to Y7 of SEQ ID NO: 1, a M at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, a Y at position P90 of SEQ ID NO: 1, and an I at position V93 of SEQ ID NO: 1.

### Y7C_I16M_P90Y_V93I_R97F

In some embodiments, a KOD polymerase variant comprises: a C at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a M at position I16 of SEQ ID NO: 1, a Y at position P90 of SEQ ID NO: 1, an I at position V93 of SEQ ID NO: 1, and a F at position R97 of SEQ ID NO: 1.

### Y7C_I16M_P90Y_V93R_R97A

In some embodiments, a KOD polymerase variant comprises: a C at a position corresponding to Y7 of SEQ ID NO: 1, a M at a position corresponding to I16 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, a M at position I16 of SEQ ID NO: 1, a Y at position P90 of SEQ ID NO: 1, a R at position V93 of SEQ ID NO: 1, and an A at position R97 of SEQ ID NO: 1.

### Y7C_I16M_V93I

In some embodiments, a KOD polymerase variant comprises: a C at a position corresponding to Y7 of SEQ ID NO: 1, a M at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and an I at position V93 of SEQ ID NO: 1. In some embodiments, a KOD polymerase variant comprises: a C at position Y7 of SEQ ID NO: 1, a M at position I16 of SEQ ID NO: 1, and an I at position V93 of SEQ ID NO: 1.

### Y7C_I16M_V93I_R97F

In some embodiments, a KOD polymerase variant comprises: a C at a position corresponding to Y7 of SEQ ID NO: 1, a M at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a M at position I16 of SEQ ID NO: 1, an I at position V93 of SEQ ID NO: 1, and a F at position R97 of SEQ ID NO: 1.

### Y7C_I16M_V93R

In some embodiments, a KOD polymerase variant comprises: a C at a position corresponding to Y7 of SEQ ID NO: 1, a M at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a M at position I16 of SEQ ID NO: 1, and a R at position V93 of SEQ ID NO: 1. In some embodiments, a KOD polymerase variant comprises: a C at position Y7 of SEQ ID NO: 1, a M at position I16 of SEQ ID NO: 1, and a R at position V93 of SEQ ID NO: 1.

### Y7C_I16W

In some embodiments, a KOD polymerase variant comprises: a C at a position corresponding to Y7 of SEQ ID NO: 1 and a W at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a W at position I16 of SEQ ID NO: 1. In some embodiments, a KOD polymerase variant comprises: a C at position Y7 of SEQ ID NO: 1, and a W at position I16 of SEQ ID NO: 1.

### Y7C_I16W_P90F

In some embodiments, a KOD polymerase variant comprises: a C at a position corresponding to Y7 of SEQ ID NO: 1, a W at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position I16 of SEQ ID NO: 1, and a F at position P90 of SEQ ID NO: 1. In some embodiments, a KOD polymerase variant comprises: a C at position Y7 of SEQ ID NO: 1, a W at position I16 of SEQ ID NO: 1, and a F at position P90 of SEQ ID NO: 1.

### Y7C_I16W_P90F_R97A

In some embodiments, a KOD polymerase variant comprises: a C at a position corresponding to Y7 of SEQ ID NO: 1, a W at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position I16 of SEQ ID NO: 1, a F at position P90 of SEQ ID NO: 1, and an A at position R97 of SEQ ID NO: 1. In some embodiments, a KOD polymerase variant comprises: a C at position Y7 of SEQ ID NO: 1, a W at position I16 of SEQ ID NO: 1, a F at position P90 of SEQ ID NO: 1, and an A at position R97 of SEQ ID NO: 1.

### Y7C_I16W_P90I

In some embodiments, a KOD polymerase variant comprises: a C at a position corresponding to Y7 of SEQ ID NO: 1, a W at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position I16 of SEQ ID NO: 1, and an I at position P90 of SEQ ID NO: 1. In some embodiments, a KOD polymerase variant comprises: a C at position Y7 of SEQ ID NO: 1, a W at position I16 of SEQ ID NO: 1, and an I at position P90 of SEQ ID NO: 1.

### Y7C_I16W_V93I

In some embodiments, a KOD polymerase variant comprises: a C at a position corresponding to Y7 of SEQ ID NO: 1, a W at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position I16 of SEQ ID NO: 1, and an I at position V93 of SEQ ID NO: 1. In some embodiments, a KOD polymerase variant comprises: a C at position Y7 of SEQ ID NO: 1, a W at position I16 of SEQ ID NO: 1, and an I at position V93 of SEQ ID NO: 1.

### Y7C_P36A

In some embodiments, a KOD polymerase variant comprises: a C at a position corresponding to Y7 of SEQ ID NO: 1 and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and an A at position P36 of SEQ ID NO: 1. In some embodiments, a KOD polymerase variant comprises: a C at position Y7 of SEQ ID NO: 1, and an A at position P36 of SEQ ID NO: 1.

### Y7C_P36A_P90F

In some embodiments, a KOD polymerase variant comprises: a C at a position corresponding to Y7 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, and a F at position P90 of SEQ ID NO: 1. In some embodiments, a KOD polymerase variant comprises: a C at position Y7 of SEQ ID NO: 1, an A at position P36 of SEQ ID NO: 1, and a F at position P90 of SEQ ID NO: 1.

### Y7C_P36A_P90F_V93I

In some embodiments, a KOD polymerase variant comprises: a C at a position corresponding to Y7 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, an A at position P36 of SEQ ID NO: 1, a F at position P90 of SEQ ID NO: 1, and an I at position V93 of SEQ ID NO: 1.

### Y7C_P36A_P90F_V93I_R97H

In some embodiments, a KOD polymerase variant comprises: a C at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7C_P36A_P90F_V93Q

In some embodiments, a KOD polymerase variant comprises: a C at a position corresponding to Y7 of SEQ ID NO: 1, an A at a position corresponding to P36 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a F at position P90 of SEQ ID NO: 1, and a Q at position V93 of SEQ ID NO: 1. In some embodiments, a KOD polymerase variant comprises: a C at position Y7 of SEQ ID NO: 1, an A at position P36 of SEQ ID NO: 1, a F at position P90 of SEQ ID NO: 1, and a Q at position V93 of SEQ ID NO: 1.

### Y7C_P36A_P90I

In some embodiments, a KOD polymerase variant comprises: a C at a position corresponding to Y7 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, and an I at position P90 of SEQ ID NO: 1. In some embodiments, a KOD polymerase variant comprises: a C at position Y7 of SEQ ID NO: 1, an A at position P36 of SEQ ID NO: 1, and an I at position P90 of SEQ ID NO: 1.

### Y7C_P36A_P90I_V93I

In some embodiments, a KOD polymerase variant comprises: a C at a position corresponding to Y7 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a C at position Y7 of SEQ ID NO: 1, an A at position P36 of SEQ ID NO: 1, an I at position P90 of SEQ ID NO: 1, and an I at position V93 of SEQ ID NO: 1.

### Y7C_P36A_P90W

In some embodiments, a KOD polymerase variant comprises: a C at a position corresponding to Y7 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, and a W at position P90 of SEQ ID NO: 1. In some embodiments, a KOD polymerase variant comprises: a C at position Y7 of SEQ ID NO: 1, an A at position P36 of SEQ ID NO: 1, and a W at position P90 of SEQ ID NO: 1.

### Y7C_P36A_P90W_V93I

In some embodiments, a KOD polymerase variant comprises: a C at a position corresponding to Y7 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, an A at position P36 of SEQ ID NO: 1, a W at position P90 of SEQ ID NO: 1, and an I at position V93 of SEQ ID NO: 1.

### Y7C_P36A_P90W_V93I_R97A

In some embodiments, a KOD polymerase variant comprises: a C at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### Y7C_P36A_P90W_V93I_R97H

In some embodiments, a KOD polymerase variant comprises: a C at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1.

### Y7C_P36A_P90W_V93R_R97A

In some embodiments, a KOD polymerase variant comprises: a C at a position corresponding to Y7 of SEQ ID NO: 1, an A at a position corresponding to P36 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, an A at position P36 of SEQ ID NO: 1, a W at position P90 of SEQ ID NO: 1, a R at position V93 of SEQ ID NO: 1, and an A at position R97 of SEQ ID NO: 1.

### Y7C_P36A_P90Y

In some embodiments, a KOD polymerase variant comprises: a C at a position corresponding to Y7 of SEQ ID NO: 1, an A at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, and a Y at position P90 of SEQ ID NO: 1. In some embodiments, a KOD polymerase variant comprises: a C at position Y7 of SEQ ID NO: 1, an A at position P36 of SEQ ID NO: 1, and a Y at position P90 of SEQ ID NO: 1.

### Y7C_P36A_P90Y_V93I

In some embodiments, a KOD polymerase variant comprises: a C at a position corresponding to Y7 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, an A at position P36 of SEQ ID NO: 1, a Y at position P90 of SEQ ID NO: 1, and an I at position V93 of SEQ ID NO: 1.

### Y7C_P36A_P90Y_V93I_R97A

In some embodiments, a KOD polymerase variant comprises: a C at a position corresponding to Y7 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7C_P36A_P90Y_V93I_R97W

In some embodiments, a KOD polymerase variant comprises: a C at a position corresponding to Y7 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1.

### Y7C_P36A_V93I

In some embodiments, a KOD polymerase variant comprises: a C at a position corresponding to Y7 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an A at position P36 of SEQ ID NO: 1, and an I at position V93 of SEQ ID NO: 1. In some embodiments, a KOD polymerase variant comprises: a C at position Y7 of SEQ ID NO: 1, an A at position P36 of SEQ ID NO: 1, and an I at position V93 of SEQ ID NO: 1.

### Y7C_P36H

In some embodiments, a KOD polymerase variant comprises: a C at a position corresponding to Y7 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a H at position P36 of SEQ ID NO: 1. In some embodiments, a KOD polymerase variant comprises: a C at position Y7 of SEQ ID NO: 1, and a H at position P36 of SEQ ID NO: 1.

### Y7C_P36H_P90F

In some embodiments, a KOD polymerase variant comprises: a C at a position corresponding to Y7 of SEQ ID NO: 1, a H at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, and a F at position P90 of SEQ ID NO: 1. In some embodiments, a KOD polymerase variant comprises: a C at position Y7 of SEQ ID NO: 1, a H at position P36 of SEQ ID NO: 1, and a F at position P90 of SEQ ID NO: 1.

### Y7C_P36H_P90F_R97H

In some embodiments, a KOD polymerase variant comprises: a C at a position corresponding to Y7 of SEQ ID NO: 1, a H at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, a F at position P90 of SEQ ID NO: 1, and a H at position R97 of SEQ ID NO: 1. In some embodiments, a KOD polymerase variant comprises: a C at position Y7 of SEQ ID NO: 1, a H at position P36 of SEQ ID NO: 1, a F at position P90 of SEQ ID NO: 1, and a H at position R97 of SEQ ID NO: 1.

### Y7C_P36H_P90F_V93I

In some embodiments, a KOD polymerase variant comprises: a C at a position corresponding to Y7 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a H at position P36 of SEQ ID NO: 1, a F at position P90 of SEQ ID NO: 1, and an I at position V93 of SEQ ID NO: 1.

### Y7C_P36H_P90F_V93I_R97H

In some embodiments, a KOD polymerase variant comprises: a C at a position corresponding to Y7 of SEQ ID NO: 1, a H at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a H at position P36 of SEQ ID NO: 1, a F at position P90 of SEQ ID NO: 1, an I at position V93 of SEQ ID NO: 1, and a H at position R97 of SEQ ID NO: 1.

### Y7C_P36H_P90W

In some embodiments, a KOD polymerase variant comprises: a C at a position corresponding to Y7 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, and a W at position P90 of SEQ ID NO: 1. In some embodiments, a KOD polymerase variant comprises: a C at position Y7 of SEQ ID NO: 1, a H at position P36 of SEQ ID NO: 1, and a W at position P90 of SEQ ID NO: 1.

### Y7C_P36H_P90W_R97H

In some embodiments, a KOD polymerase variant comprises: a C at a position corresponding to Y7 of SEQ ID NO: 1, a H at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, a W at position P90 of SEQ ID NO: 1, and a H at position R97 of SEQ ID NO: 1. In some embodiments, a KOD polymerase variant comprises: a C at position Y7 of SEQ ID NO: 1, a H at position P36 of SEQ ID NO: 1, a W at position P90 of SEQ ID NO: 1, and a H at position R97 of SEQ ID NO: 1.

### Y7C_P36H_P90W_V93I

In some embodiments, a KOD polymerase variant comprises: a C at a position corresponding to Y7 of SEQ ID NO: 1, a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a H at position P36 of SEQ ID NO: 1, a W at position P90 of SEQ ID NO: 1, and an I at position V93 of SEQ ID NO:1.

### Y7C_P36H_V93I

In some embodiments, a KOD polymerase variant comprises: a C at a position corresponding to Y7 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a H at position P36 of SEQ ID NO: 1, and an I at position V93 of SEQ ID NO: 1. In some embodiments, a KOD polymerase variant comprises: a C at position Y7 of SEQ ID NO: 1, a H at position P36 of SEQ ID NO: 1, and an I at position V93 of SEQ ID NO: 1.

### Y7C_P36R_R97A

In some embodiments, a KOD polymerase variant comprises: a C at a position corresponding to Y7 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a R at position P36 of SEQ ID NO: 1, and an A at position R97 of SEQ ID NO: 1. In some embodiments, a KOD polymerase variant comprises: a C at position Y7 of SEQ ID NO: 1, a R at position P36 of SEQ ID NO: 1, and an A at position R97 of SEQ ID NO: 1.

### Y7C_P36R_V93I_R97A

In some embodiments, a KOD polymerase variant comprises: a C at a position corresponding to Y7 of SEQ ID NO: 1, a R at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a R at position P36 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a R at position P36 of SEQ ID NO: 1, an I at position V93 of SEQ ID NO: 1, and an A at position R97 of SEQ ID NO: 1.

### Y7C_P36S_P90F_R97F

In some embodiments, a KOD polymerase variant comprises: a C at a position corresponding to Y7 of SEQ ID NO: 1, a S at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a F at position P90 of SEQ ID NO: 1, and a F at position R97 of SEQ ID NO: 1. In some embodiments, a KOD polymerase variant comprises: a C at position Y7 of SEQ ID NO: 1, a S at position P36 of SEQ ID NO: 1, a F at position P90 of SEQ ID NO: 1, and a F at position R97 of SEQ ID NO: 1.

### Y7C_P36S_P90F_V93I

In some embodiments, a KOD polymerase variant comprises: a C at a position corresponding to Y7 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a S at position P36 of SEQ ID NO: 1, a F at position P90 of SEQ ID NO: 1, and an I at position V93 of SEQ ID NO: 1.

### Y7C_P36S_P90F_V93I_R97A

In some embodiments, a KOD polymerase variant comprises: a C at a position corresponding to Y7 of SEQ ID NO: 1, a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1.

### Y7C_P36S_P90I

In some embodiments, a KOD polymerase variant comprises: a C at a position corresponding to Y7 of SEQ ID NO: 1, a S at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, and an I at position P90 of SEQ ID NO: 1. In some embodiments, a KOD polymerase variant comprises: a C at position Y7 of SEQ ID NO: 1, a S at position P36 of SEQ ID NO: 1, and an I at position P90 of SEQ ID NO: 1.

### Y7C_P36S_P90W

In some embodiments, a KOD polymerase variant comprises: a C at a position corresponding to Y7 of SEQ ID NO: 1, a S at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, and a W at position P90 of SEQ ID NO: 1. In some embodiments, a KOD polymerase variant comprises: a C at position Y7 of SEQ ID NO: 1, a S at position P36 of SEQ ID NO: 1, and a W at position P90 of SEQ ID NO: 1.

### Y7C_P36S_P90W_V93I

In some embodiments, a KOD polymerase variant comprises: a C at a position corresponding to Y7 of SEQ ID NO: 1, a S at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a S at position P36 of SEQ ID NO: 1, a W at position P90 of SEQ ID NO: 1, and an I at position V93 of SEQ ID NO: 1.

### Y7C_P36S_P90W_V93Q

In some embodiments, a KOD polymerase variant comprises: a C at a position corresponding to Y7 of SEQ ID NO: 1, a S at a position corresponding to P36 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a W at position P90 of SEQ ID NO: 1, and a Q at position V93 of SEQ ID NO: 1. In some embodiments, a KOD polymerase variant comprises: a C at position Y7 of SEQ ID NO: 1, a S at position P36 of SEQ ID NO: 1, a W at position P90 of SEQ ID NO: 1, and a Q at position V93 of SEQ ID NO: 1.

### Y7C_P36S_P90Y_R97A

In some embodiments, a KOD polymerase variant comprises: a C at a position corresponding to Y7 of SEQ ID NO: 1, a S at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a Y at position P90 of SEQ ID NO: 1, and an A at position R97 of SEQ ID NO: 1. In some embodiments, a KOD polymerase variant comprises: a C at position Y7 of SEQ ID NO: 1, a S at position P36 of SEQ ID NO: 1, a Y at position P90 of SEQ ID NO: 1, and an A at position R97 of SEQ ID NO: 1.

### Y7C_P36S_P90Y_V93I

In some embodiments, a KOD polymerase variant comprises: a C at a position corresponding to Y7 of SEQ ID NO: 1, a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P36 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a S at position P36 of SEQ ID NO: 1, a Y at position P90 of SEQ ID NO: 1, and an I at position V93 of SEQ ID NO: 1.

### Y7C_P36W

In some embodiments, a KOD polymerase variant comprises: a C at a position corresponding to Y7 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a W at position P36 of SEQ ID NO: 1. In some embodiments, a KOD polymerase variant comprises: a C at position Y7 of SEQ ID NO: 1, and a W at position P36 of SEQ ID NO: 1.

### Y7C_P36W_P90F

In some embodiments, a KOD polymerase variant comprises: a C at a position corresponding to Y7 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, and a F at position P90 of SEQ ID NO: 1. In some embodiments, a KOD polymerase variant comprises: a C at position Y7 of SEQ ID NO: 1, a W at position P36 of SEQ ID NO: 1, and a F at position P90 of SEQ ID NO: 1.

### Y7C_P36W_P90F_V93I

In some embodiments, a KOD polymerase variant comprises: a C at a position corresponding to Y7 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a F at position P90 of SEQ ID NO: 1, and an I at position V93 of SEQ ID NO: 1.

In some embodiments, a KOD polymerase variant comprises: a C at position Y7 of SEQ ID NO: 1, a W at position P36 of SEQ ID NO: 1, a F at position P90 of SEQ ID NO: 1, and an I at position V93 of SEQ ID NO: 1.

### Y7C_P36W_P90F_V93I_R97A

In some embodiments, a KOD polymerase variant comprises: a C at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7C_P36W_P90F_V93I_R97H

In some embodiments, a KOD polymerase variant comprises: a C at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### Y7C_P36W_P90W

In some embodiments, a KOD polymerase variant comprises: a C at a position corresponding to Y7 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, and a W at position P90 of SEQ ID NO: 1. In some embodiments, a KOD polymerase variant comprises: a C at position Y7 of SEQ ID NO: 1, a W at position P36 of SEQ ID NO: 1, and a W at position P90 of SEQ ID NO: 1.

### Y7C_P36W_P90W_R97A

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, and an A at position R97 of SEQ ID NO: 1.

### Y7C_P36W_P90W_R97H

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, and a H at position R97 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, and a H at position R97 of SEQ ID NO: 1.

### Y7C_P36W_P90W_V93I

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, and an I at position V93 of SEQ ID NO: 1.

### Y7C_P36W_P90W_V93I_R97A

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### Y7C_P36W_P90W_V93I_R97W

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1.

### Y7C_P36W_P90W_V93R

In some embodiments, a KOD polymerase variant comprises: a C at a position corresponding to Y7 of SEQ ID NO: 1, a W at a position corresponding to P36 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a W at position P90 of SEQ ID NO: 1, and a R at position V93 of SEQ ID NO: 1. In some embodiments, a KOD polymerase variant comprises: a C at position Y7 of SEQ ID NO: 1, a W at position P36 of SEQ ID NO: 1, a W at position P90 of SEQ ID NO: 1, and a R at position V93 of SEQ ID NO: 1.

### Y7C_P36W_P90Y

In some embodiments, a KOD polymerase variant comprises: a C at a position corresponding to Y7 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, and a Y at position P90 of SEQ ID NO: 1. In some embodiments, a KOD polymerase variant comprises: a C at position Y7 of SEQ ID NO: 1, a W at position P36 of SEQ ID NO: 1, and a Y at position P90 of SEQ ID NO: 1.

### Y7C_P36W_P90Y_V93I

In some embodiments, a KOD polymerase variant comprises: a C at a position corresponding to Y7 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a Y at position P90 of SEQ ID NO: 1, and an I at position V93 of SEQ ID NO: 1.

In some embodiments, a KOD polymerase variant comprises: a C at position Y7 of SEQ ID NO: 1, a W at position P36 of SEQ ID NO: 1, a Y at position P90 of SEQ ID NO: 1, and an I at position V93 of SEQ ID NO: 1.

### Y7C_P36W_P90Y_V93I_R97F

In some embodiments, a KOD polymerase variant comprises: a C at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1.

### Y7C_P36W_P90Y_V93I_R97H

In some embodiments, a KOD polymerase variant comprises: a C at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7C_P36W_P90Y_V93R

In some embodiments, a KOD polymerase variant comprises: a C at a position corresponding to Y7 of SEQ ID NO: 1, a W at a position corresponding to P36 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, a Y at position P90 of SEQ ID NO: 1, and a R at position V93 of SEQ ID NO: 1. In some embodiments, a KOD polymerase variant comprises: a C at position Y7 of SEQ ID NO: 1, a W at position P36 of SEQ ID NO: 1, a Y at position P90 of SEQ ID NO: 1, and a R at position V93 of SEQ ID NO: 1.

### Y7C_P36W_R97A

In some embodiments, a KOD polymerase variant comprises: a C at a position corresponding to Y7 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, and an A at position R97 of SEQ ID NO: 1. In some embodiments, a KOD polymerase variant comprises: a C at position Y7 of SEQ ID NO: 1, a W at position P36 of SEQ ID NO: 1, and an A at position R97 of SEQ ID NO: 1.

### Y7C_P36W_V93I

In some embodiments, a KOD polymerase variant comprises: a C at a position corresponding to Y7 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, and an I at position V93 of SEQ ID NO: 1. In some embodiments, a KOD polymerase variant comprises: a C at position Y7 of SEQ ID NO: 1, a W at position P36 of SEQ ID NO: 1, and an I at position V93 of SEQ ID NO: 1.

### Y7C_P36W_V93I_R97F

In some embodiments, a KOD polymerase variant comprises: a C at a position corresponding to Y7 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a W at position P36 of SEQ ID NO: 1, an I at position V93 of SEQ ID NO: 1, and a F at position R97 of SEQ ID NO: 1.

### Y7C_P90F

In some embodiments, a KOD polymerase variant comprises: a C at a position corresponding to Y7 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a F at position P90 of SEQ ID NO: 1. In some embodiments, a KOD polymerase variant comprises: a C at position Y7 of SEQ ID NO: 1, and a F at position P90 of SEQ ID NO: 1.

### Y7C_P90F_R97H

In some embodiments, a KOD polymerase variant comprises: a C at a position corresponding to Y7 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, and a H at position R97 of SEQ ID NO: 1. In some embodiments, a KOD polymerase variant comprises: a C at position Y7 of SEQ ID NO: 1, a F at position P90 of SEQ ID NO: 1, and a H at position R97 of SEQ ID NO: 1.

### Y7C_P90F_V93I

In some embodiments, a KOD polymerase variant comprises: a C at a position corresponding to Y7 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, and an I at position V93 of SEQ ID NO: 1. In some embodiments, a KOD polymerase variant comprises: a C at position Y7 of SEQ ID NO: 1, a F at position P90 of SEQ ID NO: 1, and an I at position V93 of SEQ ID NO: 1.

### Y7C_P90F_V93I_R97A

In some embodiments, a KOD polymerase variant comprises: a C at a position corresponding to Y7 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a F at position P90 of SEQ ID NO: 1, an I at position V93 of SEQ ID NO: 1, and an A at position R97 of SEQ ID NO: 1.

### Y7C_P90F_V93I_R97F

In some embodiments, a KOD polymerase variant comprises: a C at a position corresponding to Y7 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a F at position P90 of SEQ ID NO: 1, an I at position V93 of SEQ ID NO: 1, and a F at position R97 of SEQ ID NO: 1.

### Y7C_P90F_V93I_R97W

In some embodiments, a KOD polymerase variant comprises: a C at a position corresponding to Y7 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a F at position P90 of SEQ ID NO: 1, an I at position V93 of SEQ ID NO: 1, and a W at position R97 of SEQ ID NO: 1.

### Y7C_P90F_V93K

In some embodiments, a KOD polymerase variant comprises: a C at a position corresponding to Y7 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, and a K at position V93 of SEQ ID NO: 1. In some embodiments, a KOD polymerase variant comprises: a C at position Y7 of SEQ ID NO: 1, a F at position P90 of SEQ ID NO: 1, and a K at position V93 of SEQ ID NO: 1.

### Y7C_P90F_V93Q

In some embodiments, a KOD polymerase variant comprises: a C at a position corresponding to Y7 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, and a Q at position V93 of SEQ ID NO: 1. In some embodiments, a KOD polymerase variant comprises: a C at position Y7 of SEQ ID NO: 1, a F at position P90 of SEQ ID NO: 1, and a Q at position V93 of SEQ ID NO: 1.

### Y7C_P90F_V93Q_R97A

In some embodiments, a KOD polymerase variant comprises: a C at a position corresponding to Y7 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a C at position Y7 of SEQ ID NO: 1, a F at position P90 of SEQ ID NO: 1, a Q at position V93 of SEQ ID NO: 1, and an A at position R97 of SEQ ID NO: 1.

### Y7C_P90I

In some embodiments, a KOD polymerase variant comprises: a C at a position corresponding to Y7 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and an I at position P90 of SEQ ID NO: 1. In some embodiments, a KOD polymerase variant comprises: a C at position Y7 of SEQ ID NO: 1, and an I at position P90 of SEQ ID NO: 1.

### Y7C_P90I_V93I

In some embodiments, a KOD polymerase variant comprises: a C at a position corresponding to Y7 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an I at position P90 of SEQ ID NO: 1, and an I at position V93 of SEQ ID NO: 1. In some embodiments, a KOD polymerase variant comprises: a C at position Y7 of SEQ ID NO: 1, an I at position P90 of SEQ ID NO: 1, and an I at position V93 of SEQ ID NO: 1.

### Y7C_P90I_V93I_R97A

In some embodiments, a KOD polymerase variant comprises: a C at a position corresponding to Y7 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, an I at position P90 of SEQ ID NO: 1, an I at position V93 of SEQ ID NO: 1, and an A at position R97 of SEQ ID NO: 1.

### Y7C_P90I_V93I_R97F

In some embodiments, a KOD polymerase variant comprises: a C at a position corresponding to Y7 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, an I at position P90 of SEQ ID NO: 1, an I at position V93 of SEQ ID NO: 1, and a F at position R97 of SEQ ID NO: 1.

### Y7C_P90I_V93I_R97W

In some embodiments, a KOD polymerase variant comprises: a C at a position corresponding to Y7 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, an I at position P90 of SEQ ID NO: 1, an I at position V93 of SEQ ID NO: 1, and a W at position R97 of SEQ ID NO: 1.

### Y7C_P90S_V93R_R97A

In some embodiments, a KOD polymerase variant comprises: a C at a position corresponding to Y7 of SEQ ID NO: 1, a S at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a S at position P90 of SEQ ID NO: 1, a R at position V93 of SEQ ID NO: 1, and an A at position R97 of SEQ ID NO: 1. In some embodiments, a KOD polymerase variant comprises: a C at position Y7 of SEQ ID NO: 1, a S at position P90 of SEQ ID NO: 1, a R at position V93 of SEQ ID NO: 1, and an A at position R97 of SEQ ID NO: 1.

### Y7C_P90W

In some embodiments, a KOD polymerase variant comprises: a C at a position corresponding to Y7 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a W at position P90 of SEQ ID NO: 1. In some embodiments, a KOD polymerase variant comprises: a C at position Y7 of SEQ ID NO: 1, and a W at position P90 of SEQ ID NO: 1.

### Y7C_P90W_R97F

In some embodiments, a KOD polymerase variant comprises: a C at a position corresponding to Y7 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and a F at position R97 of SEQ ID NO: 1. In some embodiments, a KOD polymerase variant comprises: a C at position Y7 of SEQ ID NO: 1, a W at position P90 of SEQ ID NO: 1, and a F at position R97 of SEQ ID NO: 1.

### Y7C_P90W_R97W

In some embodiments, a KOD polymerase variant comprises: a C at a position corresponding to Y7 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and a W at position R97 of SEQ ID NO: 1. In some embodiments, a KOD polymerase variant comprises: a C at position Y7 of SEQ ID NO: 1, a W at position P90 of SEQ ID NO: 1, and a W at position R97 of SEQ ID NO: 1.

### Y7C_P90W_V93E

In some embodiments, a KOD polymerase variant comprises: a C at a position corresponding to Y7 of SEQ ID NO: 1, a W at a position corresponding to P90 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and an E at position V93 of SEQ ID NO: 1. In some embodiments, a KOD polymerase variant comprises: a C at position Y7 of SEQ ID NO: 1, a W at position P90 of SEQ ID NO: 1, and an E at position V93 of SEQ ID NO: 1.

### Y7C_P90W_V93I

In some embodiments, a KOD polymerase variant comprises: a C at a position corresponding to Y7 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and an I at position V93 of SEQ ID NO: 1. In some embodiments, a KOD polymerase variant comprises: a C at position Y7 of SEQ ID NO: 1, a W at position P90 of SEQ ID NO: 1, and an I at position V93 of SEQ ID NO: 1.

### Y7C_P90W_V93I_R97A

In some embodiments, a KOD polymerase variant comprises: a C at a position corresponding to Y7 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a C at position Y7 of SEQ ID NO: 1, a W at position P90 of SEQ ID NO: 1, an I at position V93 of SEQ ID NO: 1, and an A at position R97 of SEQ ID NO: 1.

### Y7C_P90W_V93I_R97W

In some embodiments, a KOD polymerase variant comprises: a C at a position corresponding to Y7 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, a W at position P90 of SEQ ID NO: 1, an I at position V93 of SEQ ID NO: 1, and a W at position R97 of SEQ ID NO: 1.

### Y7C_P90W_V93I_R119Q

In some embodiments, a KOD polymerase variant comprises: a C at a position corresponding to Y7 of SEQ ID NO: 1, a W at a position corresponding to P90 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, an I at position V93 of SEQ ID NO: 1, and a Q at position R119 of SEQ ID NO: 1. In some embodiments, a KOD polymerase variant comprises: a C at position Y7 of SEQ ID NO: 1, a W at position P90 of SEQ ID NO: 1, an I at position V93 of SEQ ID NO: 1, and a Q at position R119 of SEQ ID NO: 1.

### Y7C_P90W_V93K

In some embodiments, a KOD polymerase variant comprises: a C at a position corresponding to Y7 of SEQ ID NO: 1, a W at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and a K at position V93 of SEQ ID NO: 1. In some embodiments, a KOD polymerase variant comprises: a C at position Y7 of SEQ ID NO: 1, a W at position P90 of SEQ ID NO: 1, and a K at position V93 of SEQ ID NO: 1.

### Y7C_P90W_V93Q

In some embodiments, a KOD polymerase variant comprises: a C at a position corresponding to Y7 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and a Q at position V93 of SEQ ID NO: 1. In some embodiments, a KOD polymerase variant comprises: a C at position Y7 of SEQ ID NO: 1, a W at position P90 of SEQ ID NO: 1, and a Q at position V93 of SEQ ID NO: 1.

### Y7C_P90W_V93R

In some embodiments, a KOD polymerase variant comprises: a C at a position corresponding to Y7 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, and a R at position V93 of SEQ ID NO: 1. In some embodiments, a KOD polymerase variant comprises: a C at position Y7 of SEQ ID NO: 1, a W at position P90 of SEQ ID NO: 1, and a R at position V93 of SEQ ID NO: 1.

### Y7C_P90W_V93R_R97F

In some embodiments, a KOD polymerase variant comprises: a C at a position corresponding to Y7 of SEQ ID NO: 1, a W at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, a R at position V93 of SEQ ID NO: 1, and a F at position R97 of SEQ ID NO: 1. In some embodiments, a KOD polymerase variant comprises: a C at position Y7 of SEQ ID NO: 1, a W at position P90 of SEQ ID NO: 1, a R at position V93 of SEQ ID NO: 1, and a F at position R97 of SEQ ID NO:1.

### Y7C_P90W_V93R_R119Q

In some embodiments, a KOD polymerase variant comprises: a C at a position corresponding to Y7 of SEQ ID NO: 1, a W at a position corresponding to P90 of SEQ ID NO: 1, a R at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a W at position P90 of SEQ ID NO: 1, a R at position V93 of SEQ ID NO: 1, and a Q at position R119 of SEQ ID NO: 1. In some embodiments, a KOD polymerase variant comprises: a C at position Y7 of SEQ ID NO: 1, a W at position P90 of SEQ ID NO: 1, a R at position V93 of SEQ ID NO: 1, and a Q at position R119 of SEQ ID NO: 1.

### Y7C_P90Y

In some embodiments, a KOD polymerase variant comprises: a C at a position corresponding to Y7 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a Y at position P90 of SEQ ID NO: 1. In some embodiments, a KOD polymerase variant comprises: a C at position Y7 of SEQ ID NO: 1, and a Y at position P90 of SEQ ID NO: 1.

### Y7C_P90Y_R97H

In some embodiments, a KOD polymerase variant comprises: a C at a position corresponding to Y7 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, and a H at position R97 of SEQ ID NO: 1. In some embodiments, a KOD polymerase variant comprises: a C at position Y7 of SEQ ID NO: 1, a Y at position P90 of SEQ ID NO: 1, and a H at position R97 of SEQ ID NO: 1.

### Y7C_P90Y_V93I

In some embodiments, a KOD polymerase variant comprises: a C at a position corresponding to Y7 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, and an I at position V93 of SEQ ID NO: 1. In some embodiments, a KOD polymerase variant comprises: a C at position Y7 of SEQ ID NO: 1, a Y at position P90 of SEQ ID NO: 1, and an I at position V93 of SEQ ID NO: 1.

### Y7C_P90Y_V93I_R97F

In some embodiments, a KOD polymerase variant comprises: a C at a position corresponding to Y7 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a C at position Y7 of SEQ ID NO: 1, a Y at position P90 of SEQ ID NO: 1, an I at position V93 of SEQ ID NO: 1, and a F at position R97 of SEQ ID NO: 1.

### Y7C_P90Y_V93I_R97H

In some embodiments, a KOD polymerase variant comprises: a C at a position corresponding to Y7 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a C at position Y7 of SEQ ID NO: 1, a Y at position P90 of SEQ ID NO: 1, an I at position V93 of SEQ ID NO: 1, and a H at position R97 of SEQ ID NO: 1.

### Y7C_P90Y_V93K

In some embodiments, a KOD polymerase variant comprises: a C at a position corresponding to Y7 of SEQ ID NO: 1, a Y at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, and a K at position V93 of SEQ ID NO: 1. In some embodiments, a KOD polymerase variant comprises: a C at position Y7 of SEQ ID NO: 1, a Y at position P90 of SEQ ID NO: 1, and a K at position V93 of SEQ ID NO: 1.

### Y7C_P90Y_V93Q

In some embodiments, a KOD polymerase variant comprises: a C at a position corresponding to Y7 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, a Y at position P90 of SEQ ID NO: 1, and a Q at position V93 of SEQ ID NO: 1. In some embodiments, a KOD polymerase variant comprises: a C at position Y7 of SEQ ID NO: 1, a Y at position P90 of SEQ ID NO: 1, and a Q at position V93 of SEQ ID NO: 1.

### Y7C_V93I_R97W

In some embodiments, a KOD polymerase variant comprises: a C at a position corresponding to Y7 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, an I at position V93 of SEQ ID NO: 1, and a W at position R97 of SEQ ID NO: 1. In some embodiments, a KOD polymerase variant comprises: a C at position Y7 of SEQ ID NO: 1, an I at position V93 of SEQ ID NO: 1, and a W at position R97 of SEQ ID NO: 1.

### Y7C_V93K

In some embodiments, a KOD polymerase variant comprises: a C at a position corresponding to Y7 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a K at position V93 of SEQ ID NO: 1. In some embodiments, a KOD polymerase variant comprises: a C at position Y7 of SEQ ID NO: 1, and a K at position V93 of SEQ ID NO: 1.

### Y7C_V93R

In some embodiments, a KOD polymerase variant comprises: a C at a position corresponding to Y7 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a C at position Y7 of SEQ ID NO: 1, and a R at position V93 of SEQ ID NO: 1. In some embodiments, a KOD polymerase variant comprises: a C at position Y7 of SEQ ID NO: 1, and a R at position V93 of SEQ ID NO: 1.

### Y7D_I16L

In some embodiments, a KOD polymerase variant comprises: a D at a position corresponding to Y7 of SEQ ID NO: 1 and a L at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, and a L at position I16 of SEQ ID NO: 1. In some embodiments, a KOD polymerase variant comprises: a D at position Y7 of SEQ ID NO: 1, and a L at position I16 of SEQ ID NO: 1.

### Y7D_I16L_P90Y

In some embodiments, a KOD polymerase variant comprises: a D at a position corresponding to Y7 of SEQ ID NO: 1, a L at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a L at position I16 of SEQ ID NO: 1, and a Y at position P90 of SEQ ID NO: 1. In some embodiments, a KOD polymerase variant comprises: a D at position Y7 of SEQ ID NO: 1, a L at position I16 of SEQ ID NO: 1, and a Y at position P90 of SEQ ID NO: 1.

### Y7D_I16L_P90Y_V93I

In some embodiments, a KOD polymerase variant comprises: a D at a position corresponding to Y7 of SEQ ID NO: 1, a L at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a L at position I16 of SEQ ID NO: 1, a Y at position P90 of SEQ ID NO: 1, and an I at position V93 of SEQ ID NO: 1. In some embodiments, a KOD polymerase variant comprises: a D at position Y7 of SEQ ID NO: 1, a L at position I16 of SEQ ID NO: 1, a Y at position P90 of SEQ ID NO: 1, and an I at position V93 of SEQ ID NO: 1.

### Y7D_P36A_P90F_V93I_R97A_R119Q

In some embodiments, a KOD polymerase variant comprises: a D at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an A at position P36 of SEQ ID NO: 1, a F at position P90 of SEQ ID NO: 1, an I at position V93 of SEQ ID NO: 1, an A at position R97 of SEQ ID NO: 1, and a Q at position R119 of SEQ ID NO: 1. In some embodiments, a KOD polymerase variant comprises: a D at position Y7 of SEQ ID NO: 1, an A at position P36 of SEQ ID NO: 1, a F at position P90 of SEQ ID NO: 1, an I at position V93 of SEQ ID NO: 1, an A at position R97 of SEQ ID NO: 1, and a Q at position R119 of SEQ ID NO: 1.

### Y7D_P36A_P90W_V93I

In some embodiments, a KOD polymerase variant comprises: a D at a position corresponding to Y7 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a D at position Y7 of SEQ ID NO: 1, an A at position P36 of SEQ ID NO: 1, a W at position P90 of SEQ ID NO: 1, and an I at position V93 of SEQ ID NO: 1.

### Y7D_P36A_P90Y_V93I

In some embodiments, a KOD polymerase variant comprises: a D at a position corresponding to Y7 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a D at position Y7 of SEQ ID NO: 1, an A at position P36 of SEQ ID NO: 1, a Y at position P90 of SEQ ID NO: 1, and an I at position V93 of SEQ ID NO: 1.

### Y7D_P36A_V93I_R97A

In some embodiments, a KOD polymerase variant comprises: a D at a position corresponding to Y7 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a D at position Y7 of SEQ ID NO: 1, an A at position P36 of SEQ ID NO: 1, an I at position V93 of SEQ ID NO: 1, and an A at position R97 of SEQ ID NO: 1.

### Y7D_P90F_V93I

In some embodiments, a KOD polymerase variant comprises: a D at a position corresponding to Y7 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a F at position P90 of SEQ ID NO: 1, and an I at position V93 of SEQ ID NO: 1. In some embodiments, a KOD polymerase variant comprises: a D at position Y7 of SEQ ID NO: 1, a F at position P90 of SEQ ID NO: 1, and an I at position V93 of SEQ ID NO: 1.

### Y7D_P90I_V93I_R97F

In some embodiments, a KOD polymerase variant comprises: a D at a position corresponding to Y7 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a D at position Y7 of SEQ ID NO: 1, an I at position P90 of SEQ ID NO: 1, an I at position V93 of SEQ ID NO: 1, and a F at position R97 of SEQ ID NO: 1.

### Y7D_P90S_V93I

In some embodiments, a KOD polymerase variant comprises: a D at a position corresponding to Y7 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a S at position P90 of SEQ ID NO: 1, and an I at position V93 of SEQ ID NO: 1. In some embodiments, a KOD polymerase variant comprises: a D at position Y7 of SEQ ID NO: 1, a S at position P90 of SEQ ID NO: 1, and an I at position V93 of SEQ ID NO: 1.

### Y7D_P90W

In some embodiments, a KOD polymerase variant comprises: a D at a position corresponding to Y7 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, and a W at position P90 of SEQ ID NO: 1. In some embodiments, a KOD polymerase variant comprises: a D at position Y7 of SEQ ID NO: 1, and a W at position P90 of SEQ ID NO: 1.

### Y7D_P90W_V93I

In some embodiments, a KOD polymerase variant comprises: a D at a position corresponding to Y7 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a W at position P90 of SEQ ID NO: 1, and an I at position V93 of SEQ ID NO: 1. In some embodiments, a KOD polymerase variant comprises: a D at position Y7 of SEQ ID NO: 1, a W at position P90 of SEQ ID NO: 1, and an I at position V93 of SEQ ID NO: 1.

### Y7D_P90Y

In some embodiments, a KOD polymerase variant comprises: a D at a position corresponding to Y7 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, and a Y at position P90 of SEQ ID NO: 1. In some embodiments, a KOD polymerase variant comprises: a D at position Y7 of SEQ ID NO: 1, and a Y at position P90 of SEQ ID NO: 1.

### Y7D_P90Y_V93I

In some embodiments, a KOD polymerase variant comprises: a D at a position corresponding to Y7 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a Y at position P90 of SEQ ID NO: 1, and an I at position V93 of SEQ ID NO: 1. In some embodiments, a KOD polymerase variant comprises: a D at position Y7 of SEQ ID NO: 1, a Y at position P90 of SEQ ID NO: 1, and an I at position V93 of SEQ ID NO: 1.

### Y7D_P90Y_V93K

In some embodiments, a KOD polymerase variant comprises: a D at a position corresponding to Y7 of SEQ ID NO: 1, a Y at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a Y at position P90 of SEQ ID NO: 1, and a K at position V93 of SEQ ID NO: 1. In some embodiments, a KOD polymerase variant comprises: a D at position Y7 of SEQ ID NO: 1, a Y at position P90 of SEQ ID NO: 1, and a K at position V93 of SEQ ID NO: 1.

### Y7D_V93I_R97F

In some embodiments, a KOD polymerase variant comprises: a D at a position corresponding to Y7 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, an I at position V93 of SEQ ID NO: 1, and a F at position R97 of SEQ ID NO: 1. In some embodiments, a KOD polymerase variant comprises: a D at position Y7 of SEQ ID NO: 1, an I at position V93 of SEQ ID NO: 1, and a F at position R97 of SEQ ID NO: 1.

### Y7L

In some embodiments, a KOD polymerase variant comprises: a L at a position corresponding to Y7 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1. In some embodiments, a KOD polymerase variant comprises: a L at position Y7 of SEQ ID NO: 1.

### Y7L_I16F

In some embodiments, a KOD polymerase variant comprises: a L at a position corresponding to Y7 of SEQ ID NO: 1 and a F at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a F at position I16 of SEQ ID NO: 1. In some embodiments, a KOD polymerase variant comprises: a L at position Y7 of SEQ ID NO: 1, and a F at position I16 of SEQ ID NO: 1.

### Y7L_I16F_P36A

In some embodiments, a KOD polymerase variant comprises: a L at a position corresponding to Y7 of SEQ ID NO: 1, a F at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and an A at position P36 of SEQ ID NO: 1. In some embodiments, a KOD polymerase variant comprises: a L at position Y7 of SEQ ID NO: 1, a F at position I16 of SEQ ID NO: 1, and an A at position P36 of SEQ ID NO: 1.

### Y7L_I16F_P36A_P90F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and a F at position P90 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and a F at position P90 of SEQ ID NO: 1.

### Y7L_I16F_P36A_P90F_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a F at position P90 of SEQ ID NO: 1, and an A at position R97 of SEQ ID NO: 1.

### Y7L_I16F_P36A_P90F_V93I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a F at position P90 of SEQ ID NO: 1, and an I at position V93 of SEQ ID NO: 1.

### Y7L_I16F_P36A_P90F_V93I_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1.

### Y7L_I16F_P36A_P90F_V93I_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1.

### Y7L_I16F_P36A_P90F_V93I_R97H

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7L_I16F_P36A_P90I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and an I at position P90 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and an I at position P90 of SEQ ID NO: 1.

### Y7L_I16F_P36A_P90I_R97H

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position P90 of SEQ ID NO: 1, and a H at position R97 of SEQ ID NO: 1.

### Y7L_I16F_P36A_P90I_V93I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position P90 of SEQ ID NO: 1, and an I at position V93 of SEQ ID NO: 1.

### Y7L_I16F_P36A_P90W

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and a W at position P90 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and a W at position P90 of SEQ ID NO: 1.

### Y7L_I16F_P36A_P90W_V93I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1.

### Y7L_I16F_P36A_P90W_V93I_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a W at position P90 of SEQ ID NO: 1, an I at position V93 of SEQ ID NO: 1, and a F at position R97 of SEQ ID NO: 1.

### Y7L_I16F_P36A_P90W_V93I_R97H

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO:1.

### Y7L_I16F_P36A_P90Y

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and a Y at position P90 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and a Y at position P90 of SEQ ID NO: 1.

### Y7L_I16F_P36A_P90Y_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Y at position P90 of SEQ ID NO: 1, and an A at position R97 of SEQ ID NO: 1.

### Y7L_I16F_P36A_P90Y_V93I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Y at position P90 of SEQ ID NO: 1, and an I at position V93 of SEQ ID NO: 1.

### Y7L_I16F_P36A_P90Y_V93I_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7L_I16F_P36A_P90Y_V93I_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1.

### Y7L_I16F_P36A_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and an A at position R97 of SEQ ID NO: 1.

### Y7L_I16F_P36A_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and a F at position R97 of SEQ ID NO: 1.

### Y7L_I16F_P36A_V93I

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and an I at position V93 of SEQ ID NO: 1.

### Y7L_I16F_P36A_V93I_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position V93 of SEQ ID NO: 1, and an A at position R97 of SEQ ID NO: 1.

### Y7L_I16F_P36A_V93I_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1.

### Y7L_I16F_P36A_V93I_R97H

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position V93 of SEQ ID NO: 1, and a H at position R97 of SEQ ID NO: 1.

### Y7L_I16F_P36A_V93I_R97W

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, an I at position V93 of SEQ ID NO: 1, and a W at position R97 of SEQ ID NO: 1.

### Y7L_I16F_P36A_V93Q_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Q at position V93 of SEQ ID NO: 1, and an A at position R97 of SEQ ID NO: 1.

### Y7L_I16F_P36A_V93Q_R97F

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a Q at position V93 of SEQ ID NO: 1, and a F at position R97 of SEQ ID NO: 1.

### Y7L_I16F_P36A_V93R

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, and a R at position V93 of SEQ ID NO: 1.

### Y7L_I16F_P36A_V93R_R97A

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an A at position P36 of SEQ ID NO: 1, a R at position V93 of SEQ ID NO: 1, and an A at position R97 of SEQ ID NO: 1.

### Y7L_I16F_P36H

In some embodiments, a KOD polymerase variant comprises: a L at a position corresponding to Y7 of SEQ ID NO: 1, a F at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a H at position P36 of SEQ ID NO: 1. In some embodiments, a KOD polymerase variant comprises: a L at position Y7 of SEQ ID NO: 1, a F at position I16 of SEQ ID NO: 1, and a H at position P36 of SEQ ID NO: 1.

### Y7L_I16F_P36H_R97W

In some embodiments, a KOD polymerase variant comprises: a L at a position corresponding to Y7 of SEQ ID NO: 1, a F at a position corresponding to I16 of SEQ ID NO: 1, a H at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a H at position P36 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a H at position P36 of SEQ ID NO: 1, and a W at position R97 of SEQ ID NO: 1.

### Y7L_I16F_P36H_V93I

In some embodiments, a KOD polymerase variant comprises: a L at a position corresponding to Y7 of SEQ ID NO: 1, a F at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a H at position P36 of SEQ ID NO: 1, and an I at position V93 of SEQ ID NO: 1.

### Y7L_I16F_P36H_V93R

In some embodiments, a KOD polymerase variant comprises: a L at a position corresponding to Y7 of SEQ ID NO: 1, a F at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a H at position P36 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, a H at position P36 of SEQ ID NO: 1, and a R at position V93 of SEQ ID NO: 1.

### Y7L_I16F_P36R

In some embodiments, a KOD polymerase variant comprises: a L at a position corresponding to Y7 of SEQ ID NO: 1, a F at a position corresponding to I16 of SEQ ID NO: 1, and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a R at position P36 of SEQ ID NO: 1. In some embodiments, a KOD polymerase variant comprises: a L at position Y7 of SEQ ID NO: 1, a F at position I16 of SEQ ID NO: 1, and a R at position P36 of SEQ ID NO: 1.

### Y7L_I16F_P36R_R97A

In some embodiments, a KOD polymerase variant comprises: a L at a position corresponding to Y7 of SEQ ID NO: 1, a F at a position corresponding to I16 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position P36 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a R at position P36 of SEQ ID NO: 1, and an A at position R97 of SEQ ID NO: 1.

### Y7L_I16F_P36S

In some embodiments, a KOD polymerase variant comprises: a L at a position corresponding to Y7 of SEQ ID NO: 1, a F at a position corresponding to I16 of SEQ ID NO: 1, and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a S at position P36 of SEQ ID NO: 1. In some embodiments, a KOD polymerase variant comprises: a L at position Y7 of SEQ ID NO: 1, a F at position I16 of SEQ ID NO: 1, and a S at position P36 of SEQ ID NO: 1.

### Y7L_I16F_P36S_P90F_V93I

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, a F at position P90 of SEQ ID NO: 1, and an I at position V93 of SEQ ID NO: 1.

### Y7L_I16F_P36S_P90F_V93I_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, a F at position P90 of SEQ ID NO: 1, an I at position V93 of SEQ ID NO: 1, and an A at position R97 of SEQ ID NO: 1.

### Y7L_I16F_P36S_R97H

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, and a H at position R97 of SEQ ID NO: 1.

### Y7L_I16F_P36S_V93I

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, and an I at position V93 of SEQ ID NO: 1.

### Y7L_I16F_P36S_V93I_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, an I at position V93 of SEQ ID NO: 1, and an A at position R97 of SEQ ID NO: 1.

### Y7L_I16F_P36S_V93I_R97H

In some embodiments, a KOD polymerase variant comprises: a L at a position corresponding to Y7 of SEQ ID NO: 1, a F at a position corresponding to I16 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a S at position P36 of SEQ ID NO: 1, an I at position V93 of SEQ ID NO: 1, and a H at position R97 of SEQ ID NO: 1.

### Y7L_I16F_P36W

In some embodiments, a KOD polymerase variant comprises: a L at a position corresponding to Y7 of SEQ ID NO: 1, a F at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a W at position P36 of SEQ ID NO: 1. In some embodiments, a KOD polymerase variant comprises: a L at position Y7 of SEQ ID NO: 1, a F at position I16 of SEQ ID NO: 1, and a W at position P36 of SEQ ID NO: 1.

### Y7L_I16F_P36W_P90F

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, and a F at position P90 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, and a F at position P90 of SEQ ID NO: 1.

### Y7L_I16F_P36W_P90F_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a F at position P90 of SEQ ID NO: 1, and an I at position V93 of SEQ ID NO: 1.

### Y7L_I16F_P36W_P90F_V93I_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7L_I16F_P36W_P90F_V93I_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### Y7L_I16F_P36W_P90I_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, an I at position P90 of SEQ ID NO: 1, and an I at position V93 of SEQ ID NO: 1.

### Y7L_I16F_P36W_P90W

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, and a W at position P90 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, and a W at position P90 of SEQ ID NO: 1.

### Y7L_I16F_P36W_P90W_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a W at position P90 of SEQ ID NO: 1, and an A at position R97 of SEQ ID NO: 1.

### Y7L_I16F_P36W_P90W_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a W at position P90 of SEQ ID NO: 1, and a H at position R97 of SEQ ID NO: 1.

### Y7L_I16F_P36W_P90W_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a W at position P90 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a W at position P90 of SEQ ID NO: 1, and a W at position R97 of SEQ ID NO: 1.

### Y7L_I16F_P36W_P90W_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a W at position P90 of SEQ ID NO: 1, and an I at position V93 of SEQ ID NO: 1.

### Y7L_I16F_P36W_P90W_V93I_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### Y7L_I16F_P36W_P90Y_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a Y at position P90 of SEQ ID NO: 1, and an A at position R97 of SEQ ID NO: 1.

### Y7L_I16F_P36W_P90Y_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a Y at position P90 of SEQ ID NO: 1, and an I at position V93 of SEQ ID NO: 1.

### Y7L_I16F_P36W_P90Y_V93I_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7L_I16F_P36W_P90Y_V93I_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1.

### Y7L_I16F_P36W_P90Y_V93I_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7L_I16F_P36W_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, and a F at position R97 of SEQ ID NO: 1.

### Y7L_I16F_P36W_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, and a H at position R97 of SEQ ID NO: 1.

### Y7L_I16F_P36W_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, and an I at position V93 of SEQ ID NO: 1.

### Y7L_I16F_P36W_V93I_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, an I at position V93 of SEQ ID NO: 1, and an A at position R97 of SEQ ID NO: 1.

### Y7L_I16F_P36W_V93I_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, an I at position V93 of SEQ ID NO: 1, and a F at position R97 of SEQ ID NO: 1.

### Y7L_I16F_P36W_V93I_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, an I at position V93 of SEQ ID NO: 1, and a H at position R97 of SEQ ID NO: 1.

### Y7L_I16F_P36W_V93I_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P36 of SEQ ID NO: 1, an I at position V93 of SEQ ID NO: 1, and a W at position R97 of SEQ ID NO: 1.

### Y7L_I16F_P90F

In some embodiments, a KOD polymerase variant comprises: a L at a position corresponding to Y7 of SEQ ID NO: 1, a F at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a F at position P90 of SEQ ID NO: 1. In some embodiments, a KOD polymerase variant comprises: a L at position Y7 of SEQ ID NO: 1, a F at position I16 of SEQ ID NO: 1, and a F at position P90 of SEQ ID NO: 1.

### Y7L_I16F_P90F_R97A

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and an A at position R97 of SEQ ID NO: 1.

### Y7L_I16F_P90F_R97F

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a F at position R97 of SEQ ID NO: 1.

### Y7L_I16F_P90F_R97H

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a H at position R97 of SEQ ID NO: 1.

### Y7L_I16F_P90F_R97W

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a W at position R97 of SEQ ID NO: 1.

### Y7L_I16F_P90F_V93I

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and an I at position V93 of SEQ ID NO: 1.

### Y7L_I16F_P90F_V93I_R97A

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1.

### Y7L_I16F_P90F_V93I_R97F

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1.

### Y7L_I16F_P90F_V93I_R97H

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1.

### Y7L_I16F_P90F_V93I_R97W

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, an I at position V93 of SEQ ID NO: 1, and a W at position R97 of SEQ ID NO: 1.

### Y7L_I16F_P90F_V93K

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a K at position V93 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a K at position V93 of SEQ ID NO: 1.

### Y7L_I16F_P90F_V93K_R97A

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a K at position V93 of SEQ ID NO: 1, and an A at position R97 of SEQ ID NO: 1.

### Y7L_I16F_P90F_V93K_R97F

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a K at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a K at position V93 of SEQ ID NO: 1, and a F at position R97 of SEQ ID NO: 1.

### Y7L_I16F_P90F_V93Q

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a Q at position V93 of SEQ ID NO: 1.

### Y7L_I16F_P90F_V93Q_R97A

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a Q at position V93 of SEQ ID NO: 1, and an A at position R97 of SEQ ID NO: 1.

### Y7L_I16F_P90F_V93Q_R97F

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a Q at position V93 of SEQ ID NO: 1, and a F at position R97 of SEQ ID NO: 1.

### Y7L_I16F_P90F_V93Q_R97W

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a Q at position V93 of SEQ ID NO: 1, and a W at position R97 of SEQ ID NO: 1.

### Y7L_I16F_P90F_V93R

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, and a R at position V93 of SEQ ID NO: 1.

### Y7L_I16F_P90F_V93R_R97A

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a R at position V93 of SEQ ID NO: 1, and an A at position R97 of SEQ ID NO: 1.

### Y7L_I16F_P90F_V93R_R97F

In some embodiments, a KOD polymerase variant comprises: a L at a position corresponding to Y7 of SEQ ID NO: 1, a F at a position corresponding to I16 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a F at position P90 of SEQ ID NO: 1, a R at position V93 of SEQ ID NO: 1, and a F at position R97 of SEQ ID NO: 1.

### Y7L_I16F_P90I

In some embodiments, a KOD polymerase variant comprises: a L at a position corresponding to Y7 of SEQ ID NO: 1, a F at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and an I at position P90 of SEQ ID NO: 1. In some embodiments, a KOD polymerase variant comprises: a L at position Y7 of SEQ ID NO: 1, a F at position I16 of SEQ ID NO: 1, and an I at position P90 of SEQ ID NO: 1.

### Y7L_I16F_P90I_R97A

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and an A at position R97 of SEQ ID NO: 1.

### Y7L_I16F_P90I_R97F

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a F at position R97 of SEQ ID NO: 1.

### Y7L_I16F_P90I_R97H

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a H at position R97 of SEQ ID NO: 1.

### Y7L_I16F_P90I_R97W

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a W at position R97 of SEQ ID NO: 1.

### Y7L_I16F_P90I_V93I

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and an I at position V93 of SEQ ID NO: 1.

### Y7L_I16F_P90I_V93I_R97A

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1.

### Y7L_I16F_P90I_V93I_R97F

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1.

### Y7L_I16F_P90I_V93I_R97H

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1.

### Y7L_I16F_P90I_V93I_R97W

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, an I at position V93 of SEQ ID NO: 1, and a W at position R97 of SEQ ID NO: 1.

### Y7L_I16F_P90I_V93I_R119Q

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, an I at position V93 of SEQ ID NO: 1, and a Q at position R119 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, an I at position V93 of SEQ ID NO: 1, and a Q at position R119 of SEQ ID NO: 1.

### Y7L_I16F_P90I_V93K

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a K at position V93 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a K at position V93 of SEQ ID NO: 1.

### Y7L_I16F_P90I_V93K_R97A

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, a K at position V93 of SEQ ID NO: 1, and an A at position R97 of SEQ ID NO: 1.

### Y7L_I16F_P90I_V93K_R97W

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, a K at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, a K at position V93 of SEQ ID NO: 1, and a W at position R97 of SEQ ID NO: 1.

### Y7L_I16F_P90I_V93Q

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a Q at position V93 of SEQ ID NO: 1.

### Y7L_I16F_P90I_V93Q_R97F

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, a Q at position V93 of SEQ ID NO: 1, and a F at position R97 of SEQ ID NO: 1.

### Y7L_I16F_P90I_V93R

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, and a R at position V93 of SEQ ID NO: 1.

### Y7L_I16F_P90I_V93R_R97A

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, a R at position V93 of SEQ ID NO: 1, and an A at position R97 of SEQ ID NO: 1.

### Y7L_I16F_P90I_V93R_R97W

In some embodiments, a KOD polymerase variant comprises: a L at a position corresponding to Y7 of SEQ ID NO: 1, a F at a position corresponding to I16 of SEQ ID NO: 1, an I at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position P90 of SEQ ID NO: 1, a R at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an I at position P90 of SEQ ID NO: 1, a R at position V93 of SEQ ID NO: 1, and a W at position R97 of SEQ ID NO: 1.

### Y7L_I16F_P90S

In some embodiments, a KOD polymerase variant comprises: a L at a position corresponding to Y7 of SEQ ID NO: 1, a F at a position corresponding to I16 of SEQ ID NO: 1, and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a S at position P90 of SEQ ID NO: 1. In some embodiments, a KOD polymerase variant comprises: a L at position Y7 of SEQ ID NO: 1, a F at position I16 of SEQ ID NO: 1, and a S at position P90 of SEQ ID NO: 1.

### Y7L_I16F_P90S_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, and an A at position R97 of SEQ ID NO: 1.

### Y7L_I16F_P90S_R97F

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, and a F at position R97 of SEQ ID NO: 1.

### Y7L_I16F_P90S_V93I

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, and an I at position V93 of SEQ ID NO: 1.

### Y7L_I16F_P90S_V93I_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, an I at position V93 of SEQ ID NO: 1, and an A at position R97 of SEQ ID NO: 1.

### Y7L_I16F_P90S_V93I_R97F

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, an I at position V93 of SEQ ID NO: 1, and a F at position R97 of SEQ ID NO: 1.

### Y7L_I16F_P90S_V93I_R97H

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, an I at position V93 of SEQ ID NO: 1, and a H at position R97 of SEQ ID NO: 1.

### Y7L_I16F_P90S_V93I_R97W

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, an I at position V93 of SEQ ID NO: 1, and a W at position R97 of SEQ ID NO: 1.

### Y7L_I16F_P90S_V93K

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, and a K at position V93 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, and a K at position V93 of SEQ ID NO: 1.

### Y7L_I16F_P90S_V93K_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, a K at position V93 of SEQ ID NO: 1, and an A at position R97 of SEQ ID NO: 1.

### Y7L_I16F_P90S_V93Q

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, and a Q at position V93 of SEQ ID NO: 1.

### Y7L_I16F_P90S_V93Q_R97A

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, a Q at position V93 of SEQ ID NO: 1, and an A at position R97 of SEQ ID NO: 1.

### Y7L_I16F_P90S_V93R_R97F

In some embodiments, a KOD polymerase variant comprises: a L at a position corresponding to Y7 of SEQ ID NO: 1, a F at a position corresponding to I16 of SEQ ID NO: 1, a S at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a S at position P90 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a S at position P90 of SEQ ID NO: 1, a R at position V93 of SEQ ID NO: 1, and a F at position R97 of SEQ ID NO: 1.

### Y7L_I16F_P90W

In some embodiments, a KOD polymerase variant comprises: a L at a position corresponding to Y7 of SEQ ID NO: 1, a F at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a W at position P90 of SEQ ID NO: 1. In some embodiments, a KOD polymerase variant comprises: a L at position Y7 of SEQ ID NO: 1, a F at position I16 of SEQ ID NO: 1, and a W at position P90 of SEQ ID NO: 1.

### Y7L_I16F_P90W_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and an A at position R97 of SEQ ID NO: 1.

### Y7L_I16F_P90W_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and a F at position R97 of SEQ ID NO: 1.

### Y7L_I16F_P90W_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and a H at position R97 of SEQ ID NO: 1.

### Y7L_I16F_P90W_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and a W at position R97 of SEQ ID NO: 1.

### Y7L_I16F_P90W_V93I

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and an I at position V93 of SEQ ID NO: 1.

### Y7L_I16F_P90W_V93I_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1.

### Y7L_I16F_P90W_V93I_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1.

### Y7L_I16F_P90W_V93I_R97H

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1.

### Y7L_I16F_P90W_V93I_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, an I at position V93 of SEQ ID NO: 1, and a W at position R97 of SEQ ID NO: 1.

### Y7L_I16F_P90W_V93K_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a K at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a K at position V93 of SEQ ID NO: 1, and a W at position R97 of SEQ ID NO: 1.

### Y7L_I16F_P90W_V93Q

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and a Q at position V93 of SEQ ID NO: 1.

### Y7L_I16F_P90W_V93Q_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a Q at position V93 of SEQ ID NO: 1, and an A at position R97 of SEQ ID NO: 1.

### Y7L_I16F_P90W_V93Q_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a Q at position V93 of SEQ ID NO: 1, and a W at position R97 of SEQ ID NO: 1.

### Y7L_I16F_P90W_V93R

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, and a R at position V93 of SEQ ID NO: 1.

### Y7L_I16F_P90W_V93R_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a R at position V93 of SEQ ID NO: 1, and an A at position R97 of SEQ ID NO: 1.

### Y7L_I16F_P90W_V93R_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a R at position V93 of SEQ ID NO: 1, and a F at position R97 of SEQ ID NO: 1.

### Y7L_I16F_P90W_V93R_R97W

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a W at position P90 of SEQ ID NO: 1, a R at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a W at position P90 of SEQ ID NO: 1, a R at position V93 of SEQ ID NO: 1, and a W at position R97 of SEQ ID NO: 1.

### Y7L_I16F_P90Y

In some embodiments, a KOD polymerase variant comprises: a L at a position corresponding to Y7 of SEQ ID NO: 1, a F at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a Y at position P90 of SEQ ID NO: 1. In some embodiments, a KOD polymerase variant comprises: a L at position Y7 of SEQ ID NO: 1, a F at position I16 of SEQ ID NO: 1, and a Y at position P90 of SEQ ID NO: 1.

### Y7L_I16F_P90Y_R97A

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and an A at position R97 of SEQ ID NO: 1.

### Y7L_I16F_P90Y_R97F

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and a F at position R97 of SEQ ID NO: 1.

### Y7L_I16F_P90Y_R97H

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and a H at position R97 of SEQ ID NO: 1.

### Y7L_I16F_P90Y_R97W

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and a W at position R97 of SEQ ID NO: 1.

### Y7L_I16F_P90Y_V93I

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and an I at position V93 of SEQ ID NO: 1.

### Y7L_I16F_P90Y_V93I_R97A

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1.

### Y7L_I16F_P90Y_V93I_R97F

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1.

### Y7L_I16F_P90Y_V93I_R97H

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1.

### Y7L_I16F_P90Y_V93I_R97W

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1.

### Y7L_I16F_P90Y_V93K_R97A

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, a K at position V93 of SEQ ID NO: 1, and an A at position R97 of SEQ ID NO: 1.

### Y7L_I16F_P90Y_V93K_R97P

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, a K at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, a K at position V93 of SEQ ID NO: 1, and a P at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, a K at position V93 of SEQ ID NO: 1, and a P at position R97 of SEQ ID NO: 1.

### Y7L_I16F_P90Y_V93Q

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, and a Q at position V93 of SEQ ID NO: 1.

### Y7L_I16F_P90Y_V93Q_R97A

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, a Q at position V93 of SEQ ID NO: 1, and an A at position R97 of SEQ ID NO: 1.

### Y7L_I16F_P90Y_V93R_R97A

In some embodiments, a KOD polymerase variant comprises: a L at a position corresponding to Y7 of SEQ ID NO: 1, a F at a position corresponding to I16 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Y at position P90 of SEQ ID NO: 1, a R at position V93 of SEQ ID NO: 1, and an A at position R97 of SEQ ID NO: 1.

### Y7L_I16F_R97A

In some embodiments, a KOD polymerase variant comprises: a L at a position corresponding to Y7 of SEQ ID NO: 1, a F at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, and an A at position R97 of SEQ ID NO: 1.

### Y7L_I16F_R97F

In some embodiments, a KOD polymerase variant comprises: a L at a position corresponding to Y7 of SEQ ID NO: 1, a F at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, and a F at position R97 of SEQ ID NO: 1.

### Y7L_I16F_R97W

In some embodiments, a KOD polymerase variant comprises: a L at a position corresponding to Y7 of SEQ ID NO: 1, a F at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, and a W at position R97 of SEQ ID NO: 1.

### Y7L_I16F_V93E_R97A

In some embodiments, a KOD polymerase variant comprises: a L at a position corresponding to Y7 of SEQ ID NO: 1, a F at a position corresponding to I16 of SEQ ID NO: 1, an E at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an E at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an E at position V93 of SEQ ID NO: 1, and an A at position R97 of SEQ ID NO: 1.

### Y7L_I16F_V93I

In some embodiments, a KOD polymerase variant comprises: a L at a position corresponding to Y7 of SEQ ID NO: 1, a F at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and an I at position V93 of SEQ ID NO: 1. In some embodiments, a KOD polymerase variant comprises: a L at position Y7 of SEQ ID NO: 1, a F at position I16 of SEQ ID NO: 1, and an I at position V93 of SEQ ID NO: 1.

### Y7L_I16F_V93I_R97A

In some embodiments, a KOD polymerase variant comprises: a L at a position corresponding to Y7 of SEQ ID NO: 1, a F at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, an I at position V93 of SEQ ID NO: 1, and an A at position R97 of SEQ ID NO: 1.

### Y7L_I16F_V93I_R97F

In some embodiments, a KOD polymerase variant comprises: a L at a position corresponding to Y7 of SEQ ID NO: 1, a F at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, an I at position V93 of SEQ ID NO: 1, and a F at position R97 of SEQ ID NO: 1.

### Y7L_I16F_V93I_R97H

In some embodiments, a KOD polymerase variant comprises: a L at a position corresponding to Y7 of SEQ ID NO: 1, a F at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, an I at position V93 of SEQ ID NO: 1, and a H at position R97 of SEQ ID NO: 1.

### Y7L_I16F_V93I_R97W

In some embodiments, a KOD polymerase variant comprises: a L at a position corresponding to Y7 of SEQ ID NO: 1, a F at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, an I at position V93 of SEQ ID NO: 1, and a W at position R97 of SEQ ID NO: 1.

### Y7L_I16F_V93K

In some embodiments, a KOD polymerase variant comprises: a L at a position corresponding to Y7 of SEQ ID NO: 1, a F at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a K at position V93 of SEQ ID NO: 1. In some embodiments, a KOD polymerase variant comprises: a L at position Y7 of SEQ ID NO: 1, a F at position I16 of SEQ ID NO: 1, and a K at position V93 of SEQ ID NO: 1.

### Y7L_I16F_V93K_R97A

In some embodiments, a KOD polymerase variant comprises: a L at a position corresponding to Y7 of SEQ ID NO: 1, a F at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a K at position V93 of SEQ ID NO: 1, and an A at position R97 of SEQ ID NO: 1.

### Y7L_I16F_V93K_R97F

In some embodiments, a KOD polymerase variant comprises: a L at a position corresponding to Y7 of SEQ ID NO: 1, a F at a position corresponding to I16 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a K at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a K at position V93 of SEQ ID NO: 1, and a F at position R97 of SEQ ID NO: 1.

### Y7L_I16F_V93K_R97H

In some embodiments, a KOD polymerase variant comprises: a L at a position corresponding to Y7 of SEQ ID NO: 1, a F at a position corresponding to I16 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a K at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a K at position V93 of SEQ ID NO: 1, and a H at position R97 of SEQ ID NO: 1.

### Y7L_I16F_V93Q

In some embodiments, a KOD polymerase variant comprises: a L at a position corresponding to Y7 of SEQ ID NO: 1, a F at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a Q at position V93 of SEQ ID NO: 1. In some embodiments, a KOD polymerase variant comprises: a L at position Y7 of SEQ ID NO: 1, a F at position I16 of SEQ ID NO: 1, and a Q at position V93 of SEQ ID NO: 1.

### Y7L_I16F_V93Q_R97A

In some embodiments, a KOD polymerase variant comprises: a L at a position corresponding to Y7 of SEQ ID NO: 1, a F at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a Q at position V93 of SEQ ID NO: 1, and an A at position R97 of SEQ ID NO: 1.

### Y7L_I16F_V93Q_R97F

In some embodiments, a KOD polymerase variant comprises: a L at a position corresponding to Y7 of SEQ ID NO: 1, a F at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a Q at position V93 of SEQ ID NO: 1, and a F at position R97 of SEQ ID NO: 1.

### Y7L_I16F_V93Q_R97H

In some embodiments, a KOD polymerase variant comprises: a L at a position corresponding to Y7 of SEQ ID NO: 1, a F at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Q at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a Q at position V93 of SEQ ID NO: 1, and a H at position R97 of SEQ ID NO: 1.

### Y7L_I16F_V93Q_R97W

In some embodiments, a KOD polymerase variant comprises: a L at a position corresponding to Y7 of SEQ ID NO: 1, a F at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a Q at position V93 of SEQ ID NO: 1, and a W at position R97 of SEQ ID NO: 1.

### Y7L_I16F_V93R

In some embodiments, a KOD polymerase variant comprises: a L at a position corresponding to Y7 of SEQ ID NO: 1, a F at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, and a R at position V93 of SEQ ID NO: 1. In some embodiments, a KOD polymerase variant comprises: a L at position Y7 of SEQ ID NO: 1, a F at position I16 of SEQ ID NO: 1, and a R at position V93 of SEQ ID NO: 1.

### Y7L_I16F_V93R_R97A

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, and an A at position R97 of SEQ ID NO:1.

### Y7L_I16F_V93R_R97A_R119Q

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, an A at position R97 of SEQ ID NO: 1, and a Q at position R119 of SEQ ID NO: 1.

### Y7L_I16F_V93R_R97F

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, and a F at position R97 of SEQ ID NO: 1.

### Y7L_I16F_V93R_R97H

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, and a H at position R97 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, and a H at position R97 of SEQ ID NO: 1.

### Y7L_I16F_V93R_R97P

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, and a P at position R97 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, and a P at position R97 of SEQ ID NO: 1.

### Y7L_I16F_V93R_R97W

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position I16 of SEQ ID NO: 1, a R at position V93 of SEQ ID NO: 1, and a W at position R97 of SEQ ID NO: 1. In some embodiments, a KOD polymerase variant comprises: a L at position Y7 of SEQ ID NO: 1, a F at position I16 of SEQ ID NO: 1, a R at position V93 of SEQ ID NO: 1, and a W at position R97 of SEQ ID NO: 1.

### Y7L_I16H_P36H_V93I

In some embodiments, a KOD polymerase variant comprises: a L at a position corresponding to Y7 of SEQ ID NO: 1, a H at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position I16 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a H at position I16 of SEQ ID NO: 1, a H at position P36 of SEQ ID NO: 1, and an I at position V93 of SEQ ID NO: 1.

### Y7L_I16H_P36H_V93I_R97H

In some embodiments, a KOD polymerase variant comprises: a L at a position corresponding to Y7 of SEQ ID NO: 1, a H at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a H at position I16 of SEQ ID NO: 1, a H at position P36 of SEQ ID NO: 1, an I at position V93 of SEQ ID NO: 1, and a H at position R97 of SEQ ID NO: 1.

### Y7L_I16H_P36H_V93R

In some embodiments, a KOD polymerase variant comprises: a L at a position corresponding to Y7 of SEQ ID NO: 1, a H at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position I16 of SEQ ID NO: 1, a H at position P36 of SEQ ID NO: 1, and a R at position V93 of SEQ ID NO: 1. In some embodiments, a KOD polymerase variant comprises: a L at position Y7 of SEQ ID NO: 1, a H at position I16 of SEQ ID NO: 1, a H at position P36 of SEQ ID NO: 1, and a R at position V93 of SEQ ID NO: 1.

### Y7L_I16H_P90W_V93I

In some embodiments, a KOD polymerase variant comprises: a L at a position corresponding to Y7 of SEQ ID NO: 1, a H at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position I16 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a H at position I16 of SEQ ID NO: 1, a W at position P90 of SEQ ID NO: 1, and an I at position V93 of SEQ ID NO: 1.

### Y7L_I16H_P90Y_V93I

In some embodiments, a KOD polymerase variant comprises: a L at a position corresponding to Y7 of SEQ ID NO: 1, a H at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position I16 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a H at position I16 of SEQ ID NO: 1, a Y at position P90 of SEQ ID NO: 1, and an I at position V93 of SEQ ID NO: 1.

### Y7L_I16L

In some embodiments, a KOD polymerase variant comprises: a L at a position corresponding to Y7 of SEQ ID NO: 1 and a L at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a L at position I16 of SEQ ID NO: 1. In some embodiments, a KOD polymerase variant comprises: a L at position Y7 of SEQ ID NO: 1, and a L at position I16 of SEQ ID NO: 1.

### Y7L_I16L_P36A

In some embodiments, a KOD polymerase variant comprises: a L at a position corresponding to Y7 of SEQ ID NO: 1, a L at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and an A at position P36 of SEQ ID NO: 1. In some embodiments, a KOD polymerase variant comprises: a L at position Y7 of SEQ ID NO: 1, a L at position I16 of SEQ ID NO: 1, and an A at position P36 of SEQ ID NO: 1.

### Y7L_I16L_P36A_P90F

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a F at position P90 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a F at position P90 of SEQ ID NO: 1.

### Y7L_I16L_P36A_P90F_V93I

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1.

### Y7L_I16L_P36A_P90F_V93I_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and an A at position R97 of SEQ ID NO:1.

### Y7L_I16L_P36A_P90F_V93I_R97H

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7L_I16L_P36A_P90F_V93Q_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a F at position P90 of SEQ ID NO: 1, a Q at position V93 of SEQ ID NO: 1, and an A at position R97 of SEQ ID NO: 1.

### Y7L_I16L_P36A_P90W

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a W at position P90 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a W at position P90 of SEQ ID NO: 1.

### Y7L_I16L_P36A_P90W_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a W at position P90 of SEQ ID NO: 1, and an A at position R97 of SEQ ID NO: 1.

### Y7L_I16L_P36A_P90W_V93I

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1.

### Y7L_I16L_P36A_P90W_V93I_R97H

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO:1.

### Y7L_I16L_P36A_P90Y_V93I

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1.

### Y7L_I16L_P36A_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and an A at position R97 of SEQ ID NO: 1.

### Y7L_I16L_P36A_R97F

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a F at position R97 of SEQ ID NO: 1.

### Y7L_I16L_P36A_R97H

In some embodiments, a KOD polymerase variant comprises: a L at a position corresponding to Y7 of SEQ ID NO: 1, a F at a position corresponding to I16 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a H at position R97 of SEQ ID NO: 1.

### Y7L_I16L_P36A_R97W

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a W at position R97 of SEQ ID NO: 1.

### Y7L_I16L_P36A_V93I

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and an I at position V93 of SEQ ID NO:1.

### Y7L_I16L_P36A_V93I_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, an I at position V93 of SEQ ID NO: 1, and an A at position R97 of SEQ ID NO: 1.

### Y7L_I16L_P36A_V93I_R97F

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1.

### Y7L_I16L_P36A_V93I_R97H

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, an I at position V93 of SEQ ID NO: 1, and a H at position R97 of SEQ ID NO: 1.

### Y7L_I16L_P36A_V93Q

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, and a Q at position V93 of SEQ ID NO: 1.

### Y7L_I16L_P36A_V93Q_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a Q at position V93 of SEQ ID NO: 1, and an A at position R97 of SEQ ID NO: 1.

### Y7L_I16L_P36A_V93R_R97A

In some embodiments, a KOD polymerase variant comprises: a L at a position corresponding to Y7 of SEQ ID NO: 1, a L at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an A at position P36 of SEQ ID NO: 1, a R at position V93 of SEQ ID NO: 1, and an A at position R97 of SEQ ID NO: 1.

### Y7L_I16L_P36H

In some embodiments, a KOD polymerase variant comprises: a L at a position corresponding to Y7 of SEQ ID NO: 1, a L at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a H at position P36 of SEQ ID NO: 1. In some embodiments, a KOD polymerase variant comprises: a L at position Y7 of SEQ ID NO: 1, a L at position I16 of SEQ ID NO: 1, and a H at position P36 of SEQ ID NO: 1.

### Y7L_I16L_P36H_P90I_V93I

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, an I at position P90 of SEQ ID NO: 1, and an I at position V93 of SEQ ID NO: 1.

### Y7L_I16L_P36H_P90W

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, and a W at position P90 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, and a W at position P90 of SEQ ID NO: 1.

### Y7L_I16L_P36H_P90Y_V93I

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, a Y at position P90 of SEQ ID NO: 1, and an I at position V93 of SEQ ID NO: 1.

### Y7L_I16L_P36H_R97H

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, and a H at position R97 of SEQ ID NO: 1.

### Y7L_I16L_P36H_V93I

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, and an I at position V93 of SEQ ID NO: 1.

### Y7L_I16L_P36H_V93I_R97A

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, an I at position V93 of SEQ ID NO: 1, and an A at position R97 of SEQ ID NO: 1.

### Y7L_I16L_P36H_V93I_R97H

In some embodiments, a KOD polymerase variant comprises: a L at a position corresponding to Y7 of SEQ ID NO: 1, a L at a position corresponding to I16 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a H at position P36 of SEQ ID NO: 1, an I at position V93 of SEQ ID NO: 1, and a H at position R97 of SEQ ID NO: 1.

### Y7L_I16L_P36R

In some embodiments, a KOD polymerase variant comprises: a L at a position corresponding to Y7 of SEQ ID NO: 1, a L at a position corresponding to I16 of SEQ ID NO: 1, and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a R at position P36 of SEQ ID NO: 1. In some embodiments, a KOD polymerase variant comprises: a L at position Y7 of SEQ ID NO: 1, a L at position I16 of SEQ ID NO: 1, and a R at position P36 of SEQ ID NO: 1.

### Y7L_I16L_P36S

In some embodiments, a KOD polymerase variant comprises: a L at a position corresponding to Y7 of SEQ ID NO: 1, a L at a position corresponding to I16 of SEQ ID NO: 1, and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a S at position P36 of SEQ ID NO: 1. In some embodiments, a KOD polymerase variant comprises: a L at position Y7 of SEQ ID NO: 1, a L at position I16 of SEQ ID NO: 1, and a S at position P36 of SEQ ID NO: 1.

### Y7L_I16L_P36S_P90F_R97A

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, a F at position P90 of SEQ ID NO: 1, and an A at position R97 of SEQ ID NO: 1.

### Y7L_I16L_P36S_P90W_V93I_R97A

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1.

### Y7L_I16L_P36S_P90Y_V93I_R97A

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, a Y at position P90 of SEQ ID NO: 1, an I at position V93 of SEQ ID NO: 1, and an A at position R97 of SEQ ID NO: 1.

### Y7L_I16L_P36S_V93I

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, and an I at position V93 of SEQ ID NO: 1.

### Y7L_I16L_P36S_V93I_R97A

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, an I at position V93 of SEQ ID NO: 1, and an A at position R97 of SEQ ID NO: 1.

### Y7L_I16L_P36S_V93I_R97F

In some embodiments, a KOD polymerase variant comprises: a L at a position corresponding to Y7 of SEQ ID NO: 1, a L at a position corresponding to I16 of SEQ ID NO: 1, a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a S at position P36 of SEQ ID NO: 1, an I at position V93 of SEQ ID NO: 1, and a F at position R97 of SEQ ID NO: 1.

### Y7L_I16L_P36W

In some embodiments, a KOD polymerase variant comprises: a L at a position corresponding to Y7 of SEQ ID NO: 1, a L at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a W at position P36 of SEQ ID NO: 1. In some embodiments, a KOD polymerase variant comprises: a L at position Y7 of SEQ ID NO: 1, a L at position I16 of SEQ ID NO: 1, and a W at position P36 of SEQ ID NO: 1.

### Y7L_I16L_P36W_P90F

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a F at position P90 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a F at position P90 of SEQ ID NO: 1.

### Y7L_I16L_P36W_P90F_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a F at position P90 of SEQ ID NO: 1, and an A at position R97 of SEQ ID NO: 1.

### Y7L_I16L_P36W_P90F_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1.

### Y7L_I16L_P36W_P90F_V93I_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7L_I16L_P36W_P90F_V93I_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### Y7L_I16L_P36W_P90I_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position P90 of SEQ ID NO: 1, and an I at position V93 of SEQ ID NO: 1.

### Y7L_I16L_P36W_P90I_V93I_R97A

In some embodiments, a KOD polymerase variant comprises: a L at a position corresponding to Y7 of SEQ ID NO: 1, a F at a position corresponding to I16 of SEQ ID NO: 1, a W at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1.

### Y7L_I16L_P36W_P90W

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a W at position P90 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a W at position P90 of SEQ ID NO: 1.

### Y7L_I16L_P36W_P90W_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a W at position P90 of SEQ ID NO: 1, and a H at position R97 of SEQ ID NO: 1.

### Y7L_I16L_P36W_P90W_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1.

### Y7L_I16L_P36W_P90W_V93I_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1.

### Y7L_I16L_P36W_P90Y

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a Y at position P90 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a Y at position P90 of SEQ ID NO: 1.

### Y7L_I16L_P36W_P90Y_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, and an A at position R97 of SEQ ID NO: 1.

### Y7L_I16L_P36W_P90Y_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1.

### Y7L_I16L_P36W_P90Y_V93I_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7L_I16L_P36W_P90Y_V93I_R97F

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1.

### Y7L_I16L_P36W_P90Y_V93I_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7L_I16L_P36W_P90Y_V93I_R97W

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1.

### Y7L_I16L_P36W_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and an A at position R97 of SEQ ID NO: 1.

### Y7L_I16L_P36W_R97F

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a F at position R97 of SEQ ID NO: 1.

### Y7L_I16L_P36W_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a H at position R97 of SEQ ID NO: 1.

### Y7L_I16L_P36W_R97W

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a W at position R97 of SEQ ID NO: 1.

### Y7L_I16L_P36W_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and an I at position V93 of SEQ ID NO: 1.

### Y7L_I16L_P36W_V93I_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position V93 of SEQ ID NO: 1, and an A at position R97 of SEQ ID NO: 1.

### Y7L_I16L_P36W_V93I_R97F

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position V93 of SEQ ID NO: 1, and a F at position R97 of SEQ ID NO: 1.

### Y7L_I16L_P36W_V93I_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position V93 of SEQ ID NO: 1, and a H at position R97 of SEQ ID NO: 1.

### Y7L_I16L_P36W_V93I_R97W

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, an I at position V93 of SEQ ID NO: 1, and a W at position R97 of SEQ ID NO: 1.

### Y7L_I16L_P36W_V93Q

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a Q at position V93 of SEQ ID NO: 1.

### Y7L_I16L_P36W_V93Q_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, a Q at position V93 of SEQ ID NO: 1, and an A at position R97 of SEQ ID NO: 1.

### Y7L_I16L_P36W_V93R

In some embodiments, a KOD polymerase variant comprises: a L at a position corresponding to Y7 of SEQ ID NO: 1, a L at a position corresponding to I16 of SEQ ID NO: 1, a W at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P36 of SEQ ID NO: 1, and a R at position V93 of SEQ ID NO: 1. In some embodiments, a KOD polymerase variant comprises: a L at position Y7 of SEQ ID NO: 1, a L at position I16 of SEQ ID NO: 1, a W at position P36 of SEQ ID NO: 1, and a R at position V93 of SEQ ID NO: 1.

### Y7L_I16L_P90F

In some embodiments, a KOD polymerase variant comprises: a L at a position corresponding to Y7 of SEQ ID NO: 1, a L at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a F at position P90 of SEQ ID NO: 1. In some embodiments, a KOD polymerase variant comprises: a L at position Y7 of SEQ ID NO: 1, a L at position I16 of SEQ ID NO: 1, and a F at position P90 of SEQ ID NO: 1.

### Y7L_I16L_P90F_R97A

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and an A at position R97 of SEQ ID NO: 1.

### Y7L_I16L_P90F_R97F

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and a F at position R97 of SEQ ID NO: 1.

### Y7L_I16L_P90F_R97H

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and a H at position R97 of SEQ ID NO: 1.

### Y7L_I16L_P90F_R97W

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and a W at position R97 of SEQ ID NO: 1.

### Y7L_I16L_P90F_V93I

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and an I at position V93 of SEQ ID NO: 1.

### Y7L_I16L_P90F_V93I_R97A

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1.

### Y7L_I16L_P90F_V93I_R97F

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, an I at position V93 of SEQ ID NO: 1, and a F at position R97 of SEQ ID NO: 1.

### Y7L_I16L_P90F_V93I_R97H

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, an I at position V93 of SEQ ID NO: 1, and a H at position R97 of SEQ ID NO: 1.

### Y7L_I16L_P90F_V93I_R97W

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, an I at position V93 of SEQ ID NO: 1, and a W at position R97 of SEQ ID NO: 1.

### Y7L_I16L_P90F_V93Q

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, and a Q at position V93 of SEQ ID NO: 1.

### Y7L_I16L_P90F_V93R_R97F

In some embodiments, a KOD polymerase variant comprises: a L at a position corresponding to Y7 of SEQ ID NO: 1, a L at a position corresponding to I16 of SEQ ID NO: 1, a F at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a F at position P90 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a F at position P90 of SEQ ID NO: 1, a R at position V93 of SEQ ID NO: 1, and a F at position R97 of SEQ ID NO: 1.

### Y7L_I16L_P90I

In some embodiments, a KOD polymerase variant comprises: a L at a position corresponding to Y7 of SEQ ID NO: 1, a L at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and an I at position P90 of SEQ ID NO: 1. In some embodiments, a KOD polymerase variant comprises: a L at position Y7 of SEQ ID NO: 1, a L at position I16 of SEQ ID NO: 1, and an I at position P90 of SEQ ID NO: 1.

### Y7L_I16L_P90I_R97A

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, and an A at position R97 of SEQ ID NO: 1.

### Y7L_I16L_P90I_R97F

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, and a F at position R97 of SEQ ID NO: 1.

### Y7L_I16L_P90I_R97H

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, and a H at position R97 of SEQ ID NO: 1.

### Y7L_I16L_P90I_V93I

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, and an I at position V93 of SEQ ID NO: 1.

### Y7L_I16L_P90I_V93I_R97A

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1.

### Y7L_I16L_P90I_V93I_R97F

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, an I at position V93 of SEQ ID NO: 1, and a F at position R97 of SEQ ID NO: 1.

### Y7L_I16L_P90I_V93I_R97H

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, an I at position V93 of SEQ ID NO: 1, and a H at position R97 of SEQ ID NO: 1.

### Y7L_I16L_P90I_V93I_R97W

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, an I at position V93 of SEQ ID NO: 1, and a W at position R97 of SEQ ID NO: 1.

### Y7L_I16L_P90I_V93Q

In some embodiments, a KOD polymerase variant comprises: a L at a position corresponding to Y7 of SEQ ID NO: 1, a L at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position P90 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, an I at position P90 of SEQ ID NO: 1, and a Q at position V93 of SEQ ID NO: 1.

### Y7L_I16L_P90S_V93I

In some embodiments, a KOD polymerase variant comprises: a L at a position corresponding to Y7 of SEQ ID NO: 1, a L at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a S at position P90 of SEQ ID NO: 1, and an I at position V93 of SEQ ID NO: 1.

### Y7L_I16L_P90S_V93I_R97A

In some embodiments, a KOD polymerase variant comprises: a L at a position corresponding to Y7 of SEQ ID NO: 1, a L at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a S at position P90 of SEQ ID NO: 1, an I at position V93 of SEQ ID NO: 1, and an A at position R97 of SEQ ID NO: 1.

### Y7L_I16L_P90S_V93I_R97H

In some embodiments, a KOD polymerase variant comprises: a L at a position corresponding to Y7 of SEQ ID NO: 1, a L at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a S at position P90 of SEQ ID NO: 1, an I at position V93 of SEQ ID NO: 1, and a H at position R97 of SEQ ID NO: 1.

### Y7L_I16L_P90W

In some embodiments, a KOD polymerase variant comprises: a L at a position corresponding to Y7 of SEQ ID NO: 1, a L at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a W at position P90 of SEQ ID NO: 1. In some embodiments, a KOD polymerase variant comprises: a L at position Y7 of SEQ ID NO: 1, a L at position I16 of SEQ ID NO: 1, and a W at position P90 of SEQ ID NO: 1.

### Y7L_I16L_P90W_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and an A at position R97 of SEQ ID NO: 1.

### Y7L_I16L_P90W_R97F

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and a F at position R97 of SEQ ID NO: 1.

### Y7L_I16L_P90W_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and a H at position R97 of SEQ ID NO: 1.

### Y7L_I16L_P90W_R97W

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and a W at position R97 of SEQ ID NO: 1.

### Y7L_I16L_P90W_V93I

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and an I at position V93 of SEQ ID NO: 1.

### Y7L_I16L_P90W_V93I_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1.

### Y7L_I16L_P90W_V93I_R97F

In some embodiments, a KOD polymerase variant comprises: a L at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1.

### Y7L_I16L_P90W_V93I_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1.

### Y7L_I16L_P90W_V93I_R97W

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, an I at position V93 of SEQ ID NO: 1, and a W at position R97 of SEQ ID NO: 1.

### Y7L_I16L_P90W_V93K_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, a K at position V93 of SEQ ID NO: 1, and an A at position R97 of SEQ ID NO: 1.

### Y7L_I16L_P90W_V93Q

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, and a Q at position V93 of SEQ ID NO: 1.

### Y7L_I16L_P90W_V93Q_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1.

### Y7L_I16L_P90W_V93R_R97A

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1.

### Y7L_I16L_P90W_V93R_R97H

In some embodiments, a KOD polymerase variant comprises: a L at a position corresponding to Y7 of SEQ ID NO: 1, a L at a position corresponding to I16 of SEQ ID NO: 1, a W at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a W at position P90 of SEQ ID NO: 1, a R at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a W at position P90 of SEQ ID NO: 1, a R at position V93 of SEQ ID NO: 1, and a H at position R97 of SEQ ID NO: 1.

### Y7L_I16L_P90Y

In some embodiments, a KOD polymerase variant comprises: a L at a position corresponding to Y7 of SEQ ID NO: 1, a L at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a Y at position P90 of SEQ ID NO: 1. In some embodiments, a KOD polymerase variant comprises: a L at position Y7 of SEQ ID NO: 1, a L at position I16 of SEQ ID NO: 1, and a Y at position P90 of SEQ ID NO: 1.

### Y7L_I16L_P90Y_R97A

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and an A at position R97 of SEQ ID NO: 1.

### Y7L_I16L_P90Y_R97F

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and a F at position R97 of SEQ ID NO: 1.

### Y7L_I16L_P90Y_R97H

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and a H at position R97 of SEQ ID NO: 1.

### Y7L_I16L_P90Y_V93I

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and an I at position V93 of SEQ ID NO: 1.

### Y7L_I16L_P90Y_V93I_R97A

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1.

### Y7L_I16L_P90Y_V93I_R97F

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1.

### Y7L_I16L_P90Y_V93I_R97H

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, an I at position V93 of SEQ ID NO: 1, and a H at position R97 of SEQ ID NO: 1.

### Y7L_I16L_P90Y_V93I_R97W

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, an I at position V93 of SEQ ID NO: 1, and a W at position R97 of SEQ ID NO: 1.

### Y7L_I16L_P90Y_V93K

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and a K at position V93 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and a K at position V93 of SEQ ID NO: 1.

### Y7L_I16L_P90Y_V93Q

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, and a Q at position V93 of SEQ ID NO: 1.

### Y7L_I16L_P90Y_V93Q_R97F

In some embodiments, a KOD polymerase variant comprises: a L at a position corresponding to Y7 of SEQ ID NO: 1, a L at a position corresponding to I16 of SEQ ID NO: 1, a Y at a position corresponding to P90 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Y at position P90 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a Y at position P90 of SEQ ID NO: 1, a Q at position V93 of SEQ ID NO: 1, and a F at position R97 of SEQ ID NO: 1.

### Y7L_I16L_R97A

In some embodiments, a KOD polymerase variant comprises: a L at a position corresponding to Y7 of SEQ ID NO: 1, a L at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, and an A at position R97 of SEQ ID NO: 1.

### Y7L_I16L_R97F

In some embodiments, a KOD polymerase variant comprises: a L at a position corresponding to Y7 of SEQ ID NO: 1, a L at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, and a F at position R97 of SEQ ID NO: 1.

### Y7L_I16L_R97H

In some embodiments, a KOD polymerase variant comprises: a L at a position corresponding to Y7 of SEQ ID NO: 1, a L at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, and a H at position R97 of SEQ ID NO: 1.

### Y7L_I16L_R97W

In some embodiments, a KOD polymerase variant comprises: a L at a position corresponding to Y7 of SEQ ID NO: 1, a L at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, and a W at position R97 of SEQ ID NO: 1.

### Y7L_I16L_V93I

In some embodiments, a KOD polymerase variant comprises: a L at a position corresponding to Y7 of SEQ ID NO: 1, a L at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and an I at position V93 of SEQ ID NO: 1. In some embodiments, a KOD polymerase variant comprises: a L at position Y7 of SEQ ID NO: 1, a L at position I16 of SEQ ID NO: 1, and an I at position V93 of SEQ ID NO: 1.

### Y7L_I16L_V93I_R97A

In some embodiments, a KOD polymerase variant comprises: a L at a position corresponding to Y7 of SEQ ID NO: 1, a L at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, an I at position V93 of SEQ ID NO: 1, and an A at position R97 of SEQ ID NO: 1.

### Y7L_I16L_V93I_R97F

In some embodiments, a KOD polymerase variant comprises: a L at a position corresponding to Y7 of SEQ ID NO: 1, a L at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, an I at position V93 of SEQ ID NO: 1, and a F at position R97 of SEQ ID NO: 1.

### Y7L_I16L_V93I_R97H

In some embodiments, a KOD polymerase variant comprises: a L at a position corresponding to Y7 of SEQ ID NO: 1, a L at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, an I at position V93 of SEQ ID NO: 1, and a H at position R97 of SEQ ID NO: 1.

### Y7L_I16L_V93I_R97W

In some embodiments, a KOD polymerase variant comprises: a L at a position corresponding to Y7 of SEQ ID NO: 1, a F at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, an I at position V93 of SEQ ID NO: 1, and a W at position R97 of SEQ ID NO: 1.

### Y7L_I16L_V93K

In some embodiments, a KOD polymerase variant comprises: a L at a position corresponding to Y7 of SEQ ID NO: 1, a L at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a K at position V93 of SEQ ID NO: 1. In some embodiments, a KOD polymerase variant comprises: a L at position Y7 of SEQ ID NO: 1, a L at position I16 of SEQ ID NO: 1, and a K at position V93 of SEQ ID NO: 1.

### Y7L_I16L_V93Q

In some embodiments, a KOD polymerase variant comprises: a L at a position corresponding to Y7 of SEQ ID NO: 1, a L at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a Q at position V93 of SEQ ID NO: 1. In some embodiments, a KOD polymerase variant comprises: a L at position Y7 of SEQ ID NO: 1, a L at position I16 of SEQ ID NO: 1, and a Q at position V93 of SEQ ID NO: 1.

### Y7L_I16L_V93Q_R97A

In some embodiments, a KOD polymerase variant comprises: a L at a position corresponding to Y7 of SEQ ID NO: 1, a L at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a Q at position V93 of SEQ ID NO: 1, and an A at position R97 of SEQ ID NO: 1.

### Y7L_I16L_V93Q_R97F

In some embodiments, a KOD polymerase variant comprises: a L at a position corresponding to Y7 of SEQ ID NO: 1, a L at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a Q at position V93 of SEQ ID NO: 1, and a F at position R97 of SEQ ID NO: 1.

### Y7L_I16L_V93Q_R97H

In some embodiments, a KOD polymerase variant comprises: a L at a position corresponding to Y7 of SEQ ID NO: 1, a L at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Q at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a Q at position V93 of SEQ ID NO: 1, and a H at position R97 of SEQ ID NO: 1.

### Y7L_I16L_V93Q_R97W

In some embodiments, a KOD polymerase variant comprises: a L at a position corresponding to Y7 of SEQ ID NO: 1, a F at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a Q at position V93 of SEQ ID NO: 1, and a W at position R97 of SEQ ID NO: 1.

### Y7L_I16L_V93R

In some embodiments, a KOD polymerase variant comprises: a L at a position corresponding to Y7 of SEQ ID NO: 1, a L at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, and a R at position V93 of SEQ ID NO: 1. In some embodiments, a KOD polymerase variant comprises: a L at position Y7 of SEQ ID NO: 1, a L at position I16 of SEQ ID NO: 1, and a R at position V93 of SEQ ID NO: 1.

### Y7L_I16L_V93R_R97A

In some embodiments, a KOD polymerase variant comprises: a L at a position corresponding to Y7 of SEQ ID NO: 1, a L at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a L at position I16 of SEQ ID NO: 1, a R at position V93 of SEQ ID NO: 1, and an A at position R97 of SEQ ID NO: 1.

### Y7L_I16L_V93R_R97F

In some embodiments, a KOD polymerase variant comprises: a L at a position corresponding to Y7 of SEQ ID NO: 1, a F at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a L at position I16 of SEQ ID NO: 1, a R at position V93 of SEQ ID NO: 1, and a F at position R97 of SEQ ID NO: 1.

### Y7L_I16L_V93R_R97H

In some embodiments, a KOD polymerase variant comprises: a L at a position corresponding to Y7 of SEQ ID NO: 1, a L at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a R at position V93 of SEQ ID NO: 1, and a H at position R97 of SEQ ID NO: 1. In some embodiments, a KOD polymerase variant comprises: a L at position Y7 of SEQ ID NO: 1, a L at position I16 of SEQ ID NO: 1, a R at position V93 of SEQ ID NO: 1, and a H at position R97 of SEQ ID NO: 1.

### Y7L_I16L_V93R_R97W

In some embodiments, a KOD polymerase variant comprises: a L at a position corresponding to Y7 of SEQ ID NO: 1, a L at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a L at position I16 of SEQ ID NO: 1, a R at position V93 of SEQ ID NO: 1, and a W at position R97 of SEQ ID NO: 1. In some embodiments, a KOD polymerase variant comprises: a L at position Y7 of SEQ ID NO: 1, a L at position I16 of SEQ ID NO: 1, a R at position V93 of SEQ ID NO: 1, and a W at position R97 of SEQ ID NO: 1.

### Y7L_I16M

In some embodiments, a KOD polymerase variant comprises: a L at a position corresponding to Y7 of SEQ ID NO: 1 and a M at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a M at position I16 of SEQ ID NO: 1. In some embodiments, a KOD polymerase variant comprises: a L at position Y7 of SEQ ID NO: 1, and a M at position I16 of SEQ ID NO: 1.

### Y7L_I16M_P36A

In some embodiments, a KOD polymerase variant comprises: a L at a position corresponding to Y7 of SEQ ID NO: 1, a M at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and an A at position P36 of SEQ ID NO: 1. In some embodiments, a KOD polymerase variant comprises: a L at position Y7 of SEQ ID NO: 1, a M at position I16 of SEQ ID NO: 1, and an A at position P36 of SEQ ID NO: 1.

### Y7L_I16M_P36A_P90F

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, and a F at position P90 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, and a F at position P90 of SEQ ID NO: 1.

### Y7L_I16M_P36A_P90F_V93I

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, a F at position P90 of SEQ ID NO: 1, and an I at position V93 of SEQ ID NO: 1.

### Y7L_I16M_P36A_P90I_V93I_R97W

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, an I at position P90 of SEQ ID NO: 1, an I at position V93 of SEQ ID NO: 1, and a W at position R97 of SEQ ID NO: 1.

### Y7L_I16M_P36A_P90W_V93I

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1.

### Y7L_I16M_P36A_P90Y

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, and a Y at position P90 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, and a Y at position P90 of SEQ ID NO: 1.

### Y7L_I16M_P36A_P90Y_V93I

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, a Y at position P90 of SEQ ID NO: 1, and an I at position V93 of SEQ ID NO: 1.

### Y7L_I16M_P36A_P90Y_V93I_R97A

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7L_I16M_P36A_R97F

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, and a F at position R97 of SEQ ID NO: 1.

### Y7L_I16M_P36A_V93I

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, and an I at position V93 of SEQ ID NO: 1.

### Y7L_I16M_P36A_V93I_R97A

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, an I at position V93 of SEQ ID NO: 1, and an A at position R97 of SEQ ID NO: 1.

### Y7L_I16M_P36A_V93I_R97H

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, an I at position V93 of SEQ ID NO: 1, and a H at position R97 of SEQ ID NO: 1.

### Y7L_I16M_P36A_V93I_R97W

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, an I at position V93 of SEQ ID NO: 1, and a W at position R97 of SEQ ID NO: 1.

### Y7L_I16M_P36A_V93Q

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, and a Q at position V93 of SEQ ID NO: 1.

### Y7L_I16M_P36A_V93R_R97A

In some embodiments, a KOD polymerase variant comprises: a L at a position corresponding to Y7 of SEQ ID NO: 1, a M at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1.

### Y7L_I16M_P36H

In some embodiments, a KOD polymerase variant comprises: a L at a position corresponding to Y7 of SEQ ID NO: 1, a M at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a H at position P36 of SEQ ID NO: 1. In some embodiments, a KOD polymerase variant comprises: a L at position Y7 of SEQ ID NO: 1, a M at position I16 of SEQ ID NO: 1, and a H at position P36 of SEQ ID NO: 1.

### Y7L_I16M_P36H_R97A

In some embodiments, a KOD polymerase variant comprises: a L at a position corresponding to Y7 of SEQ ID NO: 1, a M at a position corresponding to I16 of SEQ ID NO: 1, a H at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a H at position P36 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a H at position P36 of SEQ ID NO: 1, and an A at position R97 of SEQ ID NO: 1.

### Y7L_I16M_P36H_V93I

In some embodiments, a KOD polymerase variant comprises: a L at a position corresponding to Y7 of SEQ ID NO: 1, a M at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a H at position P36 of SEQ ID NO: 1, and an I at position V93 of SEQ ID NO: 1.

### Y7L_I16M_P36H_V93R

In some embodiments, a KOD polymerase variant comprises: a L at a position corresponding to Y7 of SEQ ID NO: 1, a M at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a H at position P36 of SEQ ID NO: 1, and a R at position V93 of SEQ ID NO: 1. In some embodiments, a KOD polymerase variant comprises: a L at position Y7 of SEQ ID NO: 1, a M at position I16 of SEQ ID NO: 1, a H at position P36 of SEQ ID NO: 1, and a R at position V93 of SEQ ID NO: 1.

### Y7L_I16M_P36S

In some embodiments, a KOD polymerase variant comprises: a L at a position corresponding to Y7 of SEQ ID NO: 1, a M at a position corresponding to I16 of SEQ ID NO: 1, and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a S at position P36 of SEQ ID NO: 1. In some embodiments, a KOD polymerase variant comprises: a L at position Y7 of SEQ ID NO: 1, a M at position I16 of SEQ ID NO: 1, and a S at position P36 of SEQ ID NO: 1.

### Y7L_I16M_P36S_P90W

In some embodiments, a KOD polymerase variant comprises: a L at a position corresponding to Y7 of SEQ ID NO: 1, a M at a position corresponding to I16 of SEQ ID NO: 1, a S at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P36 of SEQ ID NO: 1, and a W at position P90 of SEQ ID NO: 1. In some embodiments, a KOD polymerase variant comprises: a L at position Y7 of SEQ ID NO: 1, a M at position I16 of SEQ ID NO: 1, a S at position P36 of SEQ ID NO: 1, and a W at position P90 of SEQ ID NO: 1.

### Y7L_I16M_P36S_R97A

In some embodiments, a KOD polymerase variant comprises: a L at a position corresponding to Y7 of SEQ ID NO: 1, a M at a position corresponding to I16 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P36 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a S at position P36 of SEQ ID NO: 1, and an A at position R97 of SEQ ID NO: 1.

### Y7L_I16M_P36S_V93I

In some embodiments, a KOD polymerase variant comprises: a L at a position corresponding to Y7 of SEQ ID NO: 1, a M at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P36 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a S at position P36 of SEQ ID NO: 1, and an I at position V93 of SEQ ID NO: 1.

### Y7L_I16M_P36S_V93I_R97H

In some embodiments, a KOD polymerase variant comprises: a L at a position corresponding to Y7 of SEQ ID NO: 1, a M at a position corresponding to I16 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a S at position P36 of SEQ ID NO: 1, an I at position V93 of SEQ ID NO: 1, and a H at position R97 of SEQ ID NO: 1.

### Y7L_I16M_P36W

In some embodiments, a KOD polymerase variant comprises: a L at a position corresponding to Y7 of SEQ ID NO: 1, a M at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a W at position P36 of SEQ ID NO: 1. In some embodiments, a KOD polymerase variant comprises: a L at position Y7 of SEQ ID NO: 1, a M at position I16 of SEQ ID NO: 1, and a W at position P36 of SEQ ID NO: 1.

### Y7L_I16M_P36W_P90F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a F at position P90 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a F at position P90 of SEQ ID NO: 1.

### Y7L_I16M_P36W_P90F_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, and an A at position R97 of SEQ ID NO: 1.

### Y7L_I16M_P36W_P90F_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, and a H at position R97 of SEQ ID NO: 1.

### Y7L_I16M_P36W_P90F_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1.

### Y7L_I16M_P36W_P90F_V93I_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7L_I16M_P36W_P90F_V93I_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### Y7L_I16M_P36W_P90F_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### Y7L_I16M_P36W_P90I_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position P90 of SEQ ID NO: 1, and an I at position V93 of SEQ ID NO: 1.

### Y7L_I16M_P36W_P90I_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position P90 of SEQ ID NO: 1, an I at position V93 of SEQ ID NO: 1, and a H at position R97 of SEQ ID NO: 1.

### Y7L_I16M_P36W_P90W

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a W at position P90 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a W at position P90 of SEQ ID NO: 1.

### Y7L_I16M_P36W_P90W_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a W at position P90 of SEQ ID NO: 1, and an A at position R97 of SEQ ID NO: 1.

### Y7L_I16M_P36W_P90W_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1.

### Y7L_I16M_P36W_P90W_V93I_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### Y7L_I16M_P36W_P90W_V93I_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### Y7L_I16M_P36W_P90W_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1.

### Y7L_I16M_P36W_P90Y

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a Y at position P90 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a Y at position P90 of SEQ ID NO: 1.

### Y7L_I16M_P36W_P90Y_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Y at position P90 of SEQ ID NO: 1, and an A at position R97 of SEQ ID NO: 1.

### Y7L_I16M_P36W_P90Y_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Y at position P90 of SEQ ID NO: 1, and a F at position R97 of SEQ ID NO: 1.

### Y7L_I16M_P36W_P90Y_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1.

### Y7L_I16M_P36W_P90Y_V93I_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7L_I16M_P36W_P90Y_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO:1.

### Y7L_I16M_P36W_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and an A at position R97 of SEQ ID NO:1.

### Y7L_I16M_P36W_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a F at position R97 of SEQ ID NO: 1.

### Y7L_I16M_P36W_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a H at position R97 of SEQ ID NO: 1.

### Y7L_I16M_P36W_R97W

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a W at position R97 of SEQ ID NO: 1.

### Y7L_I16M_P36W_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and an I at position V93 of SEQ ID NO: 1.

### Y7L_I16M_P36W_V93I_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1.

### Y7L_I16M_P36W_V93I_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position V93 of SEQ ID NO: 1, and a F at position R97 of SEQ ID NO: 1.

### Y7L_I16M_P36W_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position V93 of SEQ ID NO: 1, and a H at position R97 of SEQ ID NO: 1.

### Y7L_I16M_P36W_V93I_R97W

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, an I at position V93 of SEQ ID NO: 1, and a W at position R97 of SEQ ID NO: 1.

### Y7L_I16M_P36W_V93K

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a K at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a K at position V93 of SEQ ID NO: 1.

### Y7L_I16M_P36W_V93Q

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a Q at position V93 of SEQ ID NO: 1.

### Y7L_I16M_P36W_V93Q_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Q at position V93 of SEQ ID NO: 1, and an A at position R97 of SEQ ID NO: 1.

### Y7L_I16M_P36W_V93Q_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Q at position V93 of SEQ ID NO: 1, and a F at position R97 of SEQ ID NO: 1.

### Y7L_I16M_P36W_V93Q_R97W

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a Q at position V93 of SEQ ID NO: 1, and a W at position R97 of SEQ ID NO: 1.

### Y7L_I16M_P36W_V93R

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, and a R at position V93 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, and a R at position V93 of SEQ ID NO: 1.

### Y7L_I16M_P36W_V93R_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a R at position V93 of SEQ ID NO: 1, and an A at position R97 of SEQ ID NO: 1.

### Y7L_I16M_P36W_V93R_R97A_R119Q

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a R at position V93 of SEQ ID NO: 1, an A at position R97 of SEQ ID NO: 1, and a Q at position R119 of SEQ ID NO: 1.

### Y7L_I16M_P36W_V93R_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P36 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P36 of SEQ ID NO: 1, a R at position V93 of SEQ ID NO: 1, and a F at position R97 of SEQ ID NO: 1.

### Y7L_I16M_P90F

In some embodiments, a KOD polymerase variant comprises: a L at a position corresponding to Y7 of SEQ ID NO: 1, a M at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a F at position P90 of SEQ ID NO: 1. In some embodiments, a KOD polymerase variant comprises: a L at position Y7 of SEQ ID NO: 1, a M at position I16 of SEQ ID NO: 1, and a F at position P90 of SEQ ID NO: 1.

### Y7L_I16M_P90F_R97A

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and an A at position R97 of SEQ ID NO: 1.

### Y7L_I16M_P90F_R97F

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and a F at position R97 of SEQ ID NO: 1.

### Y7L_I16M_P90F_R97H

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and a H at position R97 of SEQ ID NO: 1.

### Y7L_I16M_P90F_R97W

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and a W at position R97 of SEQ ID NO: 1.

### Y7L_I16M_P90F_V93I

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and an I at position V93 of SEQ ID NO: 1.

### Y7L_I16M_P90F_V93I_R97A

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, an I at position V93 of SEQ ID NO: 1, and an A at position R97 of SEQ ID NO: 1.

### Y7L_I16M_P90F_V93I_R97F

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, an I at position V93 of SEQ ID NO: 1, and a F at position R97 of SEQ ID NO: 1.

### Y7L_I16M_P90F_V93I_R97H

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, an I at position V93 of SEQ ID NO: 1, and a H at position R97 of SEQ ID NO: 1.

### Y7L_I16M_P90F_V93I_R97W

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, an I at position V93 of SEQ ID NO: 1, and a W at position R97 of SEQ ID NO: 1.

### Y7L_I16M_P90F_V93Q

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, and a Q at position V93 of SEQ ID NO: 1.

### Y7L_I16M_P90F_V93Q_R97A

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, a Q at position V93 of SEQ ID NO: 1, and an A at position R97 of SEQ ID NO: 1.

### Y7L_I16M_P90F_V93R_R97A

In some embodiments, a KOD polymerase variant comprises: a L at a position corresponding to Y7 of SEQ ID NO: 1, a M at a position corresponding to I16 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a F at position P90 of SEQ ID NO: 1, a R at position V93 of SEQ ID NO: 1, and an A at position R97 of SEQ ID NO: 1.

### Y7L_I16M_P90I

In some embodiments, a KOD polymerase variant comprises: a L at a position corresponding to Y7 of SEQ ID NO: 1, a M at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and an I at position P90 of SEQ ID NO: 1. In some embodiments, a KOD polymerase variant comprises: a L at position Y7 of SEQ ID NO: 1, a M at position I16 of SEQ ID NO: 1, and an I at position P90 of SEQ ID NO: 1.

### Y7L_I16M_P90I_R97A

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and an A at position R97 of SEQ ID NO: 1.

### Y7L_I16M_P90I_R97F

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and a F at position R97 of SEQ ID NO: 1.

### Y7L_I16M_P90I_R97H

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and a H at position R97 of SEQ ID NO: 1.

### Y7L_I16M_P90I_R97W

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and a W at position R97 of SEQ ID NO: 1.

### Y7L_I16M_P90I_V93I

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and an I at position V93 of SEQ ID NO: 1.

### Y7L_I16M_P90I_V93I_R97A

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, an I at position V93 of SEQ ID NO: 1, and an A at position R97 of SEQ ID NO: 1.

### Y7L_I16M_P90I_V93I_R97F

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, an I at position V93 of SEQ ID NO: 1, and a F at position R97 of SEQ ID NO: 1.

### Y7L_I16M_P90I_V93I_R97H

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, an I at position V93 of SEQ ID NO: 1, and a H at position R97 of SEQ ID NO: 1.

### Y7L_I16M_P90I_V93I_R97W

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, an I at position V93 of SEQ ID NO: 1, and a W at position R97 of SEQ ID NO: 1.

### Y7L_I16M_P90I_V93Q

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and a Q at position V93 of SEQ ID NO: 1.

### Y7L_I16M_P90I_V93R

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, and a R at position V93 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, and a R at position V93 of SEQ ID NO: 1.

### Y7L_I16M_P90I_V93R_R97A

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, a R at position V93 of SEQ ID NO: 1, and an A at position R97 of SEQ ID NO: 1.

### Y7L_I16M_P90I_V93R_R97F

In some embodiments, a KOD polymerase variant comprises: a L at a position corresponding to Y7 of SEQ ID NO: 1, a M at a position corresponding to I16 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, an I at position P90 of SEQ ID NO: 1, a R at position V93 of SEQ ID NO: 1, and a F at position R97 of SEQ ID NO: 1.

### Y7L_I16M_P90S_R97F

In some embodiments, a KOD polymerase variant comprises: a L at a position corresponding to Y7 of SEQ ID NO: 1, a M at a position corresponding to I16 of SEQ ID NO: 1, a S at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a S at position P90 of SEQ ID NO: 1, and a F at position R97 of SEQ ID NO: 1.

### Y7L_I16M_P90S_V93I

In some embodiments, a KOD polymerase variant comprises: a L at a position corresponding to Y7 of SEQ ID NO: 1, a M at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a S at position P90 of SEQ ID NO: 1, and an I at position V93 of SEQ ID NO: 1.

### Y7L_I16M_P90S_V93I_R97A

In some embodiments, a KOD polymerase variant comprises: a L at a position corresponding to Y7 of SEQ ID NO: 1, a M at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a S at position P90 of SEQ ID NO: 1, an I at position V93 of SEQ ID NO: 1, and an A at position R97 of SEQ ID NO: 1.

### Y7L_I16M_P90S_V93I_R97H

In some embodiments, a KOD polymerase variant comprises: a L at a position corresponding to Y7 of SEQ ID NO: 1, a M at a position corresponding to I16 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a S at position P90 of SEQ ID NO: 1, an I at position V93 of SEQ ID NO: 1, and a H at position R97 of SEQ ID NO: 1.

### Y7L_I16M_P90W

In some embodiments, a KOD polymerase variant comprises: a L at a position corresponding to Y7 of SEQ ID NO: 1, a M at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a W at position P90 of SEQ ID NO: 1. In some embodiments, a KOD polymerase variant comprises: a L at position Y7 of SEQ ID NO: 1, a M at position I16 of SEQ ID NO: 1, and a W at position P90 of SEQ ID NO: 1.

### Y7L_I16M_P90W_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, and an A at position R97 of SEQ ID NO: 1.

### Y7L_I16M_P90W_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, and a F at position R97 of SEQ ID NO: 1.

### Y7L_I16M_P90W_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, and a H at position R97 of SEQ ID NO: 1.

### Y7L_I16M_P90W_V93I

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, and an I at position V93 of SEQ ID NO: 1.

### Y7L_I16M_P90W_V93I_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1.

### Y7L_I16M_P90W_V93I_R97F

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, an I at position V93 of SEQ ID NO: 1, and a F at position R97 of SEQ ID NO: 1.

### Y7L_I16M_P90W_V93I_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1.

### Y7L_I16M_P90W_V93I_R97W

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, an I at position V93 of SEQ ID NO: 1, and a W at position R97 of SEQ ID NO: 1.

### Y7L_I16M_P90W_V93K

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, and a K at position V93 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, and a K at position V93 of SEQ ID NO: 1.

### Y7L_I16M_P90W_V93Q_R97A

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, a Q at position V93 of SEQ ID NO: 1, and an A at position R97 of SEQ ID NO: 1.

### Y7L_I16M_P90W_V93R_R97H

In some embodiments, a KOD polymerase variant comprises: a L at a position corresponding to Y7 of SEQ ID NO: 1, a M at a position corresponding to I16 of SEQ ID NO: 1, a W at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a W at position P90 of SEQ ID NO: 1, a R at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a W at position P90 of SEQ ID NO: 1, a R at position V93 of SEQ ID NO: 1, and a H at position R97 of SEQ ID NO: 1.

### Y7L_I16M_P90Y

In some embodiments, a KOD polymerase variant comprises: a L at a position corresponding to Y7 of SEQ ID NO: 1, a M at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a Y at position P90 of SEQ ID NO: 1. In some embodiments, a KOD polymerase variant comprises: a L at position Y7 of SEQ ID NO: 1, a M at position I16 of SEQ ID NO: 1, and a Y at position P90 of SEQ ID NO: 1.

### Y7L_I16M_P90Y_R97A

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, and an A at position R97 of SEQ ID NO:1.

### Y7L_I16M_P90Y_R97F

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, and a F at position R97 of SEQ ID NO: 1.

### Y7L_I16M_P90Y_R97W

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, and a W at position R97 of SEQ ID NO: 1.

### Y7L_I16M_P90Y_V93I

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, and an I at position V93 of SEQ ID NO: 1.

### Y7L_I16M_P90Y_V93I_R97A

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, an I at position V93 of SEQ ID NO: 1, and an A at position R97 of SEQ ID NO: 1.

### Y7L_I16M_P90Y_V93I_R97F

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, an I at position V93 of SEQ ID NO: 1, and a F at position R97 of SEQ ID NO: 1.

### Y7L_I16M_P90Y_V93I_R97H

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, an I at position V93 of SEQ ID NO: 1, and a H at position R97 of SEQ ID NO: 1.

### Y7L_I16M_P90Y_V93I_R97W

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, an I at position V93 of SEQ ID NO: 1, and a W at position R97 of SEQ ID NO: 1.

### Y7L_I16M_P90Y_V93Q

In some embodiments, a KOD polymerase variant comprises: a L at a position corresponding to Y7 of SEQ ID NO: 1, a M at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Y at position P90 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, a Y at position P90 of SEQ ID NO: 1, and a Q at position V93 of SEQ ID NO: 1.

### Y7L_I16M_R97A

In some embodiments, a KOD polymerase variant comprises: a L at a position corresponding to Y7 of SEQ ID NO: 1, a M at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, and an A at position R97 of SEQ ID NO: 1.

### Y7L_I16M_R97F

In some embodiments, a KOD polymerase variant comprises: a L at a position corresponding to Y7 of SEQ ID NO: 1, a M at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, and a F at position R97 of SEQ ID NO: 1.

### Y7L_I16M_R97H

In some embodiments, a KOD polymerase variant comprises: a L at a position corresponding to Y7 of SEQ ID NO: 1, a M at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, and a H at position R97 of SEQ ID NO: 1.

### Y7L_I16M_R97W

In some embodiments, a KOD polymerase variant comprises: a L at a position corresponding to Y7 of SEQ ID NO: 1, a M at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, and a W at position R97 of SEQ ID NO: 1.

### Y7L_I16M_V93I

In some embodiments, a KOD polymerase variant comprises: a L at a position corresponding to Y7 of SEQ ID NO: 1, a M at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and an I at position V93 of SEQ ID NO: 1. In some embodiments, a KOD polymerase variant comprises: a L at position Y7 of SEQ ID NO: 1, a M at position I16 of SEQ ID NO: 1, and an I at position V93 of SEQ ID NO: 1.

### Y7L_I16M_V93I_R97A

In some embodiments, a KOD polymerase variant comprises: a L at a position corresponding to Y7 of SEQ ID NO: 1, a M at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, an I at position V93 of SEQ ID NO: 1, and an A at position R97 of SEQ ID NO: 1.

### Y7L_I16M_V93I_R97F

In some embodiments, a KOD polymerase variant comprises: a L at a position corresponding to Y7 of SEQ ID NO: 1, a M at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, an I at position V93 of SEQ ID NO: 1, and a F at position R97 of SEQ ID NO: 1.

### Y7L_I16M_V93I_R97H

In some embodiments, a KOD polymerase variant comprises: a L at a position corresponding to Y7 of SEQ ID NO: 1, a M at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, an I at position V93 of SEQ ID NO: 1, and a H at position R97 of SEQ ID NO: 1.

### Y7L_I16M_V93I_R97W

In some embodiments, a KOD polymerase variant comprises: a L at a position corresponding to Y7 of SEQ ID NO: 1, a M at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, an I at position V93 of SEQ ID NO: 1, and a W at position R97 of SEQ ID NO: 1.

### Y7L_I16M_V93K

In some embodiments, a KOD polymerase variant comprises: a L at a position corresponding to Y7 of SEQ ID NO: 1, a M at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a K at position V93 of SEQ ID NO: 1. In some embodiments, a KOD polymerase variant comprises: a L at position Y7 of SEQ ID NO: 1, a M at position I16 of SEQ ID NO: 1, and a K at position V93 of SEQ ID NO: 1.

### Y7L_I16M_V93K_R97A

In some embodiments, a KOD polymerase variant comprises: a L at a position corresponding to Y7 of SEQ ID NO: 1, a M at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a K at position V93 of SEQ ID NO: 1, and an A at position R97 of SEQ ID NO: 1.

### Y7L_I16M_V93K_R97W

In some embodiments, a KOD polymerase variant comprises: a L at a position corresponding to Y7 of SEQ ID NO: 1, a M at a position corresponding to I16 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a K at position V93 of SEQ ID NO: 1, and a W at position R97 of SEQ ID NO: 1. In some embodiments, a KOD polymerase variant comprises: a L at position Y7 of SEQ ID NO: 1, a M at position I16 of SEQ ID NO: 1, a K at position V93 of SEQ ID NO: 1, and a W at position R97 of SEQ ID NO: 1.

### Y7L_I16M_V93Q

In some embodiments, a KOD polymerase variant comprises: a L at a position corresponding to Y7 of SEQ ID NO: 1, a M at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a Q at position V93 of SEQ ID NO: 1. In some embodiments, a KOD polymerase variant comprises: a L at position Y7 of SEQ ID NO: 1, a M at position I16 of SEQ ID NO: 1, and a Q at position V93 of SEQ ID NO: 1.

### Y7L_I16M_V93Q_R97A

In some embodiments, a KOD polymerase variant comprises: a L at a position corresponding to Y7 of SEQ ID NO: 1, a M at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a Q at position V93 of SEQ ID NO: 1, and an A at position R97 of SEQ ID NO: 1.

### Y7L_I16M_V93Q_R97F

In some embodiments, a KOD polymerase variant comprises: a L at a position corresponding to Y7 of SEQ ID NO: 1, a M at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Q at position V93 of SEQ ID NO: 1, and a F at position R97 of SEQ ID NO: 1. In some embodiments, a KOD polymerase variant comprises: a L at position Y7 of SEQ ID NO: 1, a M at position I16 of SEQ ID NO: 1, a Q at position V93 of SEQ ID NO: 1, and a F at position R97 of SEQ ID NO: 1.

### Y7L_I16M_V93Q_R97H

In some embodiments, a KOD polymerase variant comprises: a L at a position corresponding to Y7 of SEQ ID NO: 1, a M at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a Q at position V93 of SEQ ID NO: 1, and a H at position R97 of SEQ ID NO: 1. In some embodiments, a KOD polymerase variant comprises: a L at position Y7 of SEQ ID NO: 1, a M at position I16 of SEQ ID NO: 1, a Q at position V93 of SEQ ID NO: 1, and a H at position R97 of SEQ ID NO: 1.

### Y7L_I16M_V93R

In some embodiments, a KOD polymerase variant comprises: a L at a position corresponding to Y7 of SEQ ID NO: 1, a M at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, and a R at position V93 of SEQ ID NO: 1. In some embodiments, a KOD polymerase variant comprises: a L at position Y7 of SEQ ID NO: 1, a M at position I16 of SEQ ID NO: 1, and a R at position V93 of SEQ ID NO: 1.

### Y7L_I16M_V93R_R97A

In some embodiments, a KOD polymerase variant comprises: a L at a position corresponding to Y7 of SEQ ID NO: 1, a M at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a M at position I16 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a M at position I16 of SEQ ID NO: 1, a R at position V93 of SEQ ID NO: 1, and an A at position R97 of SEQ ID NO: 1.

### Y7L_I16W

In some embodiments, a KOD polymerase variant comprises: a L at a position corresponding to Y7 of SEQ ID NO: 1 and a W at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a W at position I16 of SEQ ID NO: 1. In some embodiments, a KOD polymerase variant comprises: a L at position Y7 of SEQ ID NO: 1, and a W at position I16 of SEQ ID NO: 1.

### Y7L_I16W_P36A

In some embodiments, a KOD polymerase variant comprises: a L at a position corresponding to Y7 of SEQ ID NO: 1, a W at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and an A at position P36 of SEQ ID NO: 1. In some embodiments, a KOD polymerase variant comprises: a L at position Y7 of SEQ ID NO: 1, a W at position I16 of SEQ ID NO: 1, and an A at position P36 of SEQ ID NO: 1.

### Y7L_I16W_P36A_R97A

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, and an A at position R97 of SEQ ID NO: 1.

### Y7L_I16W_P36A_V93I

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, and an I at position V93 of SEQ ID NO: 1.

### Y7L_I16W_P36A_V93I_R97A

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, an I at position V93 of SEQ ID NO: 1, and an A at position R97 of SEQ ID NO: 1.

### Y7L_I16W_P36A_V93I_R97F

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, an I at position V93 of SEQ ID NO: 1, and a F at position R97 of SEQ ID NO: 1.

### Y7L_I16W_P36A_V93I_R97H

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, an I at position V93 of SEQ ID NO: 1, and a H at position R97 of SEQ ID NO: 1.

### Y7L_I16W_P36A_V93I_R97W

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, an I at position V93 of SEQ ID NO: 1, and a W at position R97 of SEQ ID NO: 1.

### Y7L_I16W_P36A_V93R

In some embodiments, a KOD polymerase variant comprises: a L at a position corresponding to Y7 of SEQ ID NO: 1, a W at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an A at position P36 of SEQ ID NO: 1, and a R at position V93 of SEQ ID NO: 1. In some embodiments, a KOD polymerase variant comprises: a L at position Y7 of SEQ ID NO: 1, a W at position I16 of SEQ ID NO: 1, an A at position P36 of SEQ ID NO: 1, and a R at position V93 of SEQ ID NO: 1.

### Y7L_I16W_P36H

In some embodiments, a KOD polymerase variant comprises: a L at a position corresponding to Y7 of SEQ ID NO: 1, a W at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a H at position P36 of SEQ ID NO: 1. In some embodiments, a KOD polymerase variant comprises: a L at position Y7 of SEQ ID NO: 1, a W at position I16 of SEQ ID NO: 1, and a H at position P36 of SEQ ID NO: 1.

### Y7L_I16W_P36H_V93I

In some embodiments, a KOD polymerase variant comprises: a L at a position corresponding to Y7 of SEQ ID NO: 1, a W at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a H at position P36 of SEQ ID NO: 1, and an I at position V93 of SEQ ID NO: 1.

### Y7L_I16W_P36S_V93I

In some embodiments, a KOD polymerase variant comprises: a L at a position corresponding to Y7 of SEQ ID NO: 1, a W at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a S at position P36 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a S at position P36 of SEQ ID NO: 1, and an I at position V93 of SEQ ID NO: 1.

### Y7L_I16W_P36W_P90Y_V93I

In some embodiments, a KOD polymerase variant comprises: a L at a position corresponding to Y7 of SEQ ID NO: 1, a W at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a W at position P36 of SEQ ID NO: 1, a Y at position P90 of SEQ ID NO: 1, and an I at position V93 of SEQ ID NO: 1.

### Y7L_I16W_P36W_R97A

In some embodiments, a KOD polymerase variant comprises: a L at a position corresponding to Y7 of SEQ ID NO: 1, a W at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P36 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, a W at position P36 of SEQ ID NO: 1, and an A at position R97 of SEQ ID NO: 1.

### Y7L_I16W_P36W_V93I

In some embodiments, a KOD polymerase variant comprises: a L at a position corresponding to Y7 of SEQ ID NO: 1, a W at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P36 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a W at position P36 of SEQ ID NO: 1, and an I at position V93 of SEQ ID NO: 1.

### Y7L_I16W_P90F

In some embodiments, a KOD polymerase variant comprises: a L at a position corresponding to Y7 of SEQ ID NO: 1, a W at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a F at position P90 of SEQ ID NO: 1. In some embodiments, a KOD polymerase variant comprises: a L at position Y7 of SEQ ID NO: 1, a W at position I16 of SEQ ID NO: 1, and a F at position P90 of SEQ ID NO: 1.

### Y7L_I16W_P90F_R97A

In some embodiments, a KOD polymerase variant comprises: a L at a position corresponding to Y7 of SEQ ID NO: 1, a W at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, a F at position P90 of SEQ ID NO: 1, and an A at position R97 of SEQ ID NO: 1.

### Y7L_I16W_P90F_V93I

In some embodiments, a KOD polymerase variant comprises: a L at a position corresponding to Y7 of SEQ ID NO: 1, a W at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a F at position P90 of SEQ ID NO: 1, and an I at position V93 of SEQ ID NO: 1.

### Y7L_I16W_P90F_V93I_R97A

In some embodiments, a KOD polymerase variant comprises: a L at a position corresponding to Y7 of SEQ ID NO: 1, a W at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, a F at position P90 of SEQ ID NO: 1, an I at position V93 of SEQ ID NO: 1, and an A at position R97 of SEQ ID NO: 1.

### Y7L_I16W_P90F_V93I_R97H

In some embodiments, a KOD polymerase variant comprises: a L at a position corresponding to Y7 of SEQ ID NO: 1, a W at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position I16 of SEQ ID NO: 1, a F at position P90 of SEQ ID NO: 1, an I at position V93 of SEQ ID NO: 1, and a H at position R97 of SEQ ID NO: 1.

### Y7L_I16W_P90I

In some embodiments, a KOD polymerase variant comprises: a L at a position corresponding to Y7 of SEQ ID NO: 1, a W at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and an I at position P90 of SEQ ID NO: 1. In some embodiments, a KOD polymerase variant comprises: a L at position Y7 of SEQ ID NO: 1, a W at position I16 of SEQ ID NO: 1, and an I at position P90 of SEQ ID NO: 1.

### Y7L_I16W_P90I_V93I

In some embodiments, a KOD polymerase variant comprises: a L at a position corresponding to Y7 of SEQ ID NO: 1, a W at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, an I at position P90 of SEQ ID NO: 1, and an I at position V93 of SEQ ID NO: 1.

### Y7L_I16W_P90W

In some embodiments, a KOD polymerase variant comprises: a L at a position corresponding to Y7 of SEQ ID NO: 1, a W at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a W at position P90 of SEQ ID NO: 1. In some embodiments, a KOD polymerase variant comprises: a L at position Y7 of SEQ ID NO: 1, a W at position I16 of SEQ ID NO: 1, and a W at position P90 of SEQ ID NO: 1.

### Y7L_I16W_P90W_V93I

In some embodiments, a KOD polymerase variant comprises: a L at a position corresponding to Y7 of SEQ ID NO: 1, a W at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a W at position P90 of SEQ ID NO: 1, and an I at position V93 of SEQ ID NO: 1.

### Y7L_I16W_P90W_V93I_R97A

In some embodiments, a KOD polymerase variant comprises: a L at a position corresponding to Y7 of SEQ ID NO: 1, a W at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, a W at position P90 of SEQ ID NO: 1, an I at position V93 of SEQ ID NO: 1, and an A at position R97 of SEQ ID NO: 1.

### Y7L_I16W_P90W_V93I_R97W

In some embodiments, a KOD polymerase variant comprises: a L at a position corresponding to Y7 of SEQ ID NO: 1, a W at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position I16 of SEQ ID NO: 1, a W at position P90 of SEQ ID NO: 1, an I at position V93 of SEQ ID NO: 1, and a W at position R97 of SEQ ID NO: 1.

### Y7L_I16W_P90Y_V93I

In some embodiments, a KOD polymerase variant comprises: a L at a position corresponding to Y7 of SEQ ID NO: 1, a W at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, a Y at position P90 of SEQ ID NO: 1, and an I at position V93 of SEQ ID NO: 1.

### Y7L_I16W_R97A

In some embodiments, a KOD polymerase variant comprises: a L at a position corresponding to Y7 of SEQ ID NO: 1, a W at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and an A at position R97 of SEQ ID NO: 1. In some embodiments, a KOD polymerase variant comprises: a L at position Y7 of SEQ ID NO: 1, a W at position I16 of SEQ ID NO: 1, and an A at position R97 of SEQ ID NO: 1.

### Y7L_I16W_R97F

In some embodiments, a KOD polymerase variant comprises: a L at a position corresponding to Y7 of SEQ ID NO: 1, a W at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a F at position R97 of SEQ ID NO: 1. In some embodiments, a KOD polymerase variant comprises: a L at position Y7 of SEQ ID NO: 1, a W at position I16 of SEQ ID NO: 1, and a F at position R97 of SEQ ID NO: 1.

### Y7L_I16W_R97H

In some embodiments, a KOD polymerase variant comprises: a L at a position corresponding to Y7 of SEQ ID NO: 1, a W at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a H at position R97 of SEQ ID NO: 1. In some embodiments, a KOD polymerase variant comprises: a L at position Y7 of SEQ ID NO: 1, a W at position I16 of SEQ ID NO: 1, and a H at position R97 of SEQ ID NO: 1.

### Y7L_I16W_R97W

In some embodiments, a KOD polymerase variant comprises: a L at a position corresponding to Y7 of SEQ ID NO: 1, a W at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a W at position R97 of SEQ ID NO: 1. In some embodiments, a KOD polymerase variant comprises: a L at position Y7 of SEQ ID NO: 1, a W at position I16 of SEQ ID NO: 1, and a W at position R97 of SEQ ID NO: 1.

### Y7L_I16W_V93I

In some embodiments, a KOD polymerase variant comprises: a L at a position corresponding to Y7 of SEQ ID NO: 1, a W at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and an I at position V93 of SEQ ID NO: 1. In some embodiments, a KOD polymerase variant comprises: a L at position Y7 of SEQ ID NO: 1, a W at position I16 of SEQ ID NO: 1, and an I at position V93 of SEQ ID NO: 1.

### Y7L_I16W_V93I_R97F

In some embodiments, a KOD polymerase variant comprises: a L at a position corresponding to Y7 of SEQ ID NO: 1, a W at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position I16 of SEQ ID NO: 1, an I at position V93 of SEQ ID NO: 1, and a F at position R97 of SEQ ID NO: 1.

### Y7L_I16W_V93I_R97H

In some embodiments, a KOD polymerase variant comprises: a L at a position corresponding to Y7 of SEQ ID NO: 1, a W at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position I16 of SEQ ID NO: 1, an I at position V93 of SEQ ID NO: 1, and a H at position R97 of SEQ ID NO: 1.

### Y7L_I16W_V93I_R97W

In some embodiments, a KOD polymerase variant comprises: a L at a position corresponding to Y7 of SEQ ID NO: 1, a W at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position I16 of SEQ ID NO: 1, an I at position V93 of SEQ ID NO: 1, and a W at position R97 of SEQ ID NO: 1.

### Y7L_I16W_V93Q

In some embodiments, a KOD polymerase variant comprises: a L at a position corresponding to Y7 of SEQ ID NO: 1, a W at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a Q at position V93 of SEQ ID NO: 1. In some embodiments, a KOD polymerase variant comprises: a L at position Y7 of SEQ ID NO: 1, a W at position I16 of SEQ ID NO: 1, and a Q at position V93 of SEQ ID NO: 1.

### Y7L_I16W_V93R

In some embodiments, a KOD polymerase variant comprises: a L at a position corresponding to Y7 of SEQ ID NO: 1, a W at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position I16 of SEQ ID NO: 1, and a R at position V93 of SEQ ID NO: 1. In some embodiments, a KOD polymerase variant comprises: a L at position Y7 of SEQ ID NO: 1, a W at position I16 of SEQ ID NO: 1, and a R at position V93 of SEQ ID NO: 1.

### Y7L_P36A

In some embodiments, a KOD polymerase variant comprises: a L at a position corresponding to Y7 of SEQ ID NO: 1 and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and an A at position P36 of SEQ ID NO: 1. In some embodiments, a KOD polymerase variant comprises: a L at position Y7 of SEQ ID NO: 1, and an A at position P36 of SEQ ID NO: 1.

### Y7L_P36A_P90F

In some embodiments, a KOD polymerase variant comprises: a L at a position corresponding to Y7 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and a F at position P90 of SEQ ID NO: 1. In some embodiments, a KOD polymerase variant comprises: a L at position Y7 of SEQ ID NO: 1, an A at position P36 of SEQ ID NO: 1, and a F at position P90 of SEQ ID NO: 1.

### Y7L_P36A_P90F_R97A

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, and an A at position R97 of SEQ ID NO: 1.

### Y7L_P36A_P90F_R97F

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, and a F at position R97 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, and a F at position R97 of SEQ ID NO: 1.

### Y7L_P36A_P90F_V93I

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, and an I at position V93 of SEQ ID NO: 1.

### Y7L_P36A_P90F_V93I_R97F

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, an I at position V93 of SEQ ID NO: 1, and a F at position R97 of SEQ ID NO: 1.

### Y7L_P36A_P90F_V93I_R97H

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7L_P36A_P90F_V93I_R97W

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, an I at position V93 of SEQ ID NO: 1, and a W at position R97 of SEQ ID NO: 1.

### Y7L_P36A_P90F_V93R

In some embodiments, a KOD polymerase variant comprises: a L at a position corresponding to Y7 of SEQ ID NO: 1, an A at a position corresponding to P36 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a F at position P90 of SEQ ID NO: 1, and a R at position V93 of SEQ ID NO: 1. In some embodiments, a KOD polymerase variant comprises: a L at position Y7 of SEQ ID NO: 1, an A at position P36 of SEQ ID NO: 1, a F at position P90 of SEQ ID NO: 1, and a R at position V93 of SEQ ID NO: 1.

### Y7L_P36A_P90I

In some embodiments, a KOD polymerase variant comprises: a L at a position corresponding to Y7 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and an I at position P90 of SEQ ID NO: 1. In some embodiments, a KOD polymerase variant comprises: a L at position Y7 of SEQ ID NO: 1, an A at position P36 of SEQ ID NO: 1, and an I at position P90 of SEQ ID NO: 1.

### Y7L_P36A_P90I_R97F

In some embodiments, a KOD polymerase variant comprises: a L at a position corresponding to Y7 of SEQ ID NO: 1, an A at a position corresponding to P36 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, an I at position P90 of SEQ ID NO: 1, and a F at position R97 of SEQ ID NO: 1. In some embodiments, a KOD polymerase variant comprises: a L at position Y7 of SEQ ID NO: 1, an A at position P36 of SEQ ID NO: 1, an I at position P90 of SEQ ID NO: 1, and a F at position R97 of SEQ ID NO: 1.

### Y7L_P36A_P90I_V93I

In some embodiments, a KOD polymerase variant comprises: a L at a position corresponding to Y7 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, an A at position P36 of SEQ ID NO: 1, an I at position P90 of SEQ ID NO: 1, and an I at position V93 of SEQ ID NO: 1.

### Y7L_P36A_P90W

In some embodiments, a KOD polymerase variant comprises: a L at a position corresponding to Y7 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and a W at position P90 of SEQ ID NO: 1. In some embodiments, a KOD polymerase variant comprises: a L at position Y7 of SEQ ID NO: 1, an A at position P36 of SEQ ID NO: 1, and a W at position P90 of SEQ ID NO: 1.

### Y7L_P36A_P90W_R97A

In some embodiments, a KOD polymerase variant comprises: a L at a position corresponding to Y7 of SEQ ID NO: 1, an A at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, a W at position P90 of SEQ ID NO: 1, and an A at position R97 of SEQ ID NO: 1.

### Y7L_P36A_P90W_V93I

In some embodiments, a KOD polymerase variant comprises: a L at a position corresponding to Y7 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, an A at position P36 of SEQ ID NO: 1, a W at position P90 of SEQ ID NO: 1, and an I at position V93 of SEQ ID NO: 1.

### Y7L_P36A_P90W_V93I_R97A

In some embodiments, a KOD polymerase variant comprises: a L at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### Y7L_P36A_P90W_V93I_R97H

In some embodiments, a KOD polymerase variant comprises: a L at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, an A at position P36 of SEQ ID NO: 1, a W at position P90 of SEQ ID NO: 1, an I at position V93 of SEQ ID NO: 1, and a H at position R97 of SEQ ID NO: 1.

### Y7L_P36A_P90W_V93I_R97W

In some embodiments, a KOD polymerase variant comprises: a L at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, an A at position P36 of SEQ ID NO: 1, a W at position P90 of SEQ ID NO: 1, an I at position V93 of SEQ ID NO: 1, and a W at position R97 of SEQ ID NO: 1.

### Y7L_P36A_P90Y_V93I

In some embodiments, a KOD polymerase variant comprises: a L at a position corresponding to Y7 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, an A at position P36 of SEQ ID NO: 1, a Y at position P90 of SEQ ID NO: 1, and an I at position V93 of SEQ ID NO: 1.

### Y7L_P36A_P90Y_V93I_R97A

In some embodiments, a KOD polymerase variant comprises: a L at a position corresponding to Y7 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7L_P36A_P90Y_V93I_R97F

In some embodiments, a KOD polymerase variant comprises: a L at a position corresponding to Y7 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, an A at position P36 of SEQ ID NO: 1, a Y at position P90 of SEQ ID NO: 1, an I at position V93 of SEQ ID NO: 1, and a F at position R97 of SEQ ID NO: 1.

### Y7L_P36A_R97A

In some embodiments, a KOD polymerase variant comprises: a L at a position corresponding to Y7 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, and an A at position R97 of SEQ ID NO: 1.

### Y7L_P36A_R97H

In some embodiments, a KOD polymerase variant comprises: a L at a position corresponding to Y7 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and a H at position R97 of SEQ ID NO: 1. In some embodiments, a KOD polymerase variant comprises: a L at position Y7 of SEQ ID NO: 1, an A at position P36 of SEQ ID NO: 1, and a H at position R97 of SEQ ID NO: 1.

### Y7L_P36A_V93I

In some embodiments, a KOD polymerase variant comprises: a L at a position corresponding to Y7 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and an I at position V93 of SEQ ID NO: 1. In some embodiments, a KOD polymerase variant comprises: a L at position Y7 of SEQ ID NO: 1, an A at position P36 of SEQ ID NO: 1, and an I at position V93 of SEQ ID NO: 1.

### Y7L_P36A_V93I_R97A

In some embodiments, a KOD polymerase variant comprises: a L at a position corresponding to Y7 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, an I at position V93 of SEQ ID NO: 1, and an A at position R97 of SEQ ID NO: 1.

### Y7L_P36A_V93I_R97F

In some embodiments, a KOD polymerase variant comprises: a L at a position corresponding to Y7 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, an A at position P36 of SEQ ID NO: 1, an I at position V93 of SEQ ID NO: 1, and a F at position R97 of SEQ ID NO: 1.

### Y7L_P36A_V93I_R97H

In some embodiments, a KOD polymerase variant comprises: a L at a position corresponding to Y7 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, an A at position P36 of SEQ ID NO: 1, an I at position V93 of SEQ ID NO: 1, and a H at position R97 of SEQ ID NO: 1.

### Y7L_P36A_V93Q

In some embodiments, a KOD polymerase variant comprises: a L at a position corresponding to Y7 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and a Q at position V93 of SEQ ID NO: 1. In some embodiments, a KOD polymerase variant comprises: a L at position Y7 of SEQ ID NO: 1, an A at position P36 of SEQ ID NO: 1, and a Q at position V93 of SEQ ID NO: 1.

### Y7L_P36A_V93Q_R97A

In some embodiments, a KOD polymerase variant comprises: a L at a position corresponding to Y7 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, an A at position P36 of SEQ ID NO: 1, a Q at position V93 of SEQ ID NO: 1, and an A at position R97 of SEQ ID NO: 1.

### Y7L_P36A_V93R

In some embodiments, a KOD polymerase variant comprises: a L at a position corresponding to Y7 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an A at position P36 of SEQ ID NO: 1, and a R at position V93 of SEQ ID NO: 1. In some embodiments, a KOD polymerase variant comprises: a L at position Y7 of SEQ ID NO: 1, an A at position P36 of SEQ ID NO: 1, and a R at position V93 of SEQ ID NO: 1.

### Y7L_P36H

In some embodiments, a KOD polymerase variant comprises: a L at a position corresponding to Y7 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a H at position P36 of SEQ ID NO: 1. In some embodiments, a KOD polymerase variant comprises: a L at position Y7 of SEQ ID NO: 1, and a H at position P36 of SEQ ID NO: 1.

### Y7L_P36H_P90F

In some embodiments, a KOD polymerase variant comprises: a L at a position corresponding to Y7 of SEQ ID NO: 1, a H at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, and a F at position P90 of SEQ ID NO: 1. In some embodiments, a KOD polymerase variant comprises: a L at position Y7 of SEQ ID NO: 1, a H at position P36 of SEQ ID NO: 1, and a F at position P90 of SEQ ID NO: 1.

### Y7L_P36H_P90F_R97H

In some embodiments, a KOD polymerase variant comprises: a L at a position corresponding to Y7 of SEQ ID NO: 1, a H at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a H at position P36 of SEQ ID NO: 1, a F at position P90 of SEQ ID NO: 1, and a H at position R97 of SEQ ID NO: 1.

### Y7L_P36H_P90F_V93I

In some embodiments, a KOD polymerase variant comprises: a L at a position corresponding to Y7 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a H at position P36 of SEQ ID NO: 1, a F at position P90 of SEQ ID NO: 1, and an I at position V93 of SEQ ID NO: 1.

### Y7L_P36H_P90W

In some embodiments, a KOD polymerase variant comprises: a L at a position corresponding to Y7 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, and a W at position P90 of SEQ ID NO: 1. In some embodiments, a KOD polymerase variant comprises: a L at position Y7 of SEQ ID NO: 1, a H at position P36 of SEQ ID NO: 1, and a W at position P90 of SEQ ID NO: 1.

### Y7L_P36H_P90W_R97A

In some embodiments, a KOD polymerase variant comprises: a L at a position corresponding to Y7 of SEQ ID NO: 1, a H at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a H at position P36 of SEQ ID NO: 1, a W at position P90 of SEQ ID NO: 1, and an A at position R97 of SEQ ID NO: 1.

### Y7L_P36H_P90W_V93I

In some embodiments, a KOD polymerase variant comprises: a L at a position corresponding to Y7 of SEQ ID NO: 1, a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a H at position P36 of SEQ ID NO: 1, a W at position P90 of SEQ ID NO: 1, and an I at position V93 of SEQ ID NO: 1.

### Y7L_P36H_P90Y_V93I

In some embodiments, a KOD polymerase variant comprises: a L at a position corresponding to Y7 of SEQ ID NO: 1, a H at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a H at position P36 of SEQ ID NO: 1, a Y at position P90 of SEQ ID NO: 1, and an I at position V93 of SEQ ID NO: 1.

### Y7L_P36H_V93I

In some embodiments, a KOD polymerase variant comprises: a L at a position corresponding to Y7 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, and an I at position V93 of SEQ ID NO: 1. In some embodiments, a KOD polymerase variant comprises: a L at position Y7 of SEQ ID NO: 1, a H at position P36 of SEQ ID NO: 1, and an I at position V93 of SEQ ID NO: 1.

### Y7L_P36H_V93I_R97F

In some embodiments, a KOD polymerase variant comprises: a L at a position corresponding to Y7 of SEQ ID NO: 1, a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a H at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a H at position P36 of SEQ ID NO: 1, an I at position V93 of SEQ ID NO: 1, and a F at position R97 of SEQ ID NO: 1.

### Y7L_P36R

In some embodiments, a KOD polymerase variant comprises: a L at a position corresponding to Y7 of SEQ ID NO: 1 and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a R at position P36 of SEQ ID NO: 1. In some embodiments, a KOD polymerase variant comprises: a L at position Y7 of SEQ ID NO: 1, and a R at position P36 of SEQ ID NO: 1.

### Y7L_P36R_R97A

In some embodiments, a KOD polymerase variant comprises: a L at a position corresponding to Y7 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a R at position P36 of SEQ ID NO: 1, and an A at position R97 of SEQ ID NO: 1. In some embodiments, a KOD polymerase variant comprises: a L at position Y7 of SEQ ID NO: 1, a R at position P36 of SEQ ID NO: 1, and an A at position R97 of SEQ ID NO: 1.

### Y7L_P36S

In some embodiments, a KOD polymerase variant comprises: a L at a position corresponding to Y7 of SEQ ID NO: 1 and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a S at position P36 of SEQ ID NO: 1. In some embodiments, a KOD polymerase variant comprises: a L at position Y7 of SEQ ID NO: 1, and a S at position P36 of SEQ ID NO: 1.

### Y7L_P36S_P90F_V93I

In some embodiments, a KOD polymerase variant comprises: a L at a position corresponding to Y7 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a S at position P36 of SEQ ID NO: 1, a F at position P90 of SEQ ID NO: 1, and an I at position V93 of SEQ ID NO: 1.

### Y7L_P36S_P90I_V93I_R97F

In some embodiments, a KOD polymerase variant comprises: a L at a position corresponding to Y7 of SEQ ID NO: 1, a S at a position corresponding to P36 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a S at position P36 of SEQ ID NO: 1, an I at position P90 of SEQ ID NO: 1, an I at position V93 of SEQ ID NO: 1, and a F at position R97 of SEQ ID NO: 1.

### Y7L_P36S_P90W_R97A

In some embodiments, a KOD polymerase variant comprises: a L at a position corresponding to Y7 of SEQ ID NO: 1, a S at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a S at position P36 of SEQ ID NO: 1, a W at position P90 of SEQ ID NO: 1, and an A at position R97 of SEQ ID NO:1.

### Y7L_P36S_P90W_R97F

In some embodiments, a KOD polymerase variant comprises: a L at a position corresponding to Y7 of SEQ ID NO: 1, a S at a position corresponding to P36 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a S at position P36 of SEQ ID NO: 1, a W at position P90 of SEQ ID NO: 1, and a F at position R97 of SEQ ID NO: 1.

### Y7L_P36S_P90W_V93I

In some embodiments, a KOD polymerase variant comprises: a L at a position corresponding to Y7 of SEQ ID NO: 1, a S at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a S at position P36 of SEQ ID NO: 1, a W at position P90 of SEQ ID NO: 1, and an I at position V93 of SEQ ID NO: 1.

### Y7L_P36S_P90W_V93I_R97A

In some embodiments, a KOD polymerase variant comprises: a L at a position corresponding to Y7 of SEQ ID NO: 1, a S at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a S at position P36 of SEQ ID NO: 1, a W at position P90 of SEQ ID NO: 1, an I at position V93 of SEQ ID NO: 1, and an A at position R97 of SEQ ID NO: 1.

### Y7L_P36S_P90W_V93R_R97A

In some embodiments, a KOD polymerase variant comprises: a L at a position corresponding to Y7 of SEQ ID NO: 1, a S at a position corresponding to P36 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a S at position P36 of SEQ ID NO: 1, a W at position P90 of SEQ ID NO: 1, a R at position V93 of SEQ ID NO: 1, and an A at position R97 of SEQ ID NO: 1.

### Y7L_P36S_P90Y

In some embodiments, a KOD polymerase variant comprises: a L at a position corresponding to Y7 of SEQ ID NO: 1, a S at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, and a Y at position P90 of SEQ ID NO: 1. In some embodiments, a KOD polymerase variant comprises: a L at position Y7 of SEQ ID NO: 1, a S at position P36 of SEQ ID NO: 1, and a Y at position P90 of SEQ ID NO: 1.

### Y7L_P36S_P90Y_V93I

In some embodiments, a KOD polymerase variant comprises: a L at a position corresponding to Y7 of SEQ ID NO: 1, a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a S at position P36 of SEQ ID NO: 1, a Y at position P90 of SEQ ID NO: 1, and an I at position V93 of SEQ ID NO: 1.

### Y7L_P36S_P90Y_V93I_R97A

In some embodiments, a KOD polymerase variant comprises: a L at a position corresponding to Y7 of SEQ ID NO: 1, a S at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a S at position P36 of SEQ ID NO: 1, a Y at position P90 of SEQ ID NO: 1, an I at position V93 of SEQ ID NO: 1, and an A at position R97 of SEQ ID NO: 1.

### Y7L_P36S_V93I

In some embodiments, a KOD polymerase variant comprises: a L at a position corresponding to Y7 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, and an I at position V93 of SEQ ID NO: 1. In some embodiments, a KOD polymerase variant comprises: a L at position Y7 of SEQ ID NO: 1, a S at position P36 of SEQ ID NO: 1, and an I at position V93 of SEQ ID NO: 1.

### Y7L_P36S_V93I_R97W

In some embodiments, a KOD polymerase variant comprises: a L at a position corresponding to Y7 of SEQ ID NO: 1, a S at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a S at position P36 of SEQ ID NO: 1, an I at position V93 of SEQ ID NO: 1, and a W at position R97 of SEQ ID NO: 1.

### Y7L_P36S_V93Q_R97A

In some embodiments, a KOD polymerase variant comprises: a L at a position corresponding to Y7 of SEQ ID NO: 1, a S at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P36 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a S at position P36 of SEQ ID NO: 1, a Q at position V93 of SEQ ID NO: 1, and an A at position R97 of SEQ ID NO: 1.

### Y7L_P36W

In some embodiments, a KOD polymerase variant comprises: a L at a position corresponding to Y7 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a W at position P36 of SEQ ID NO: 1. In some embodiments, a KOD polymerase variant comprises: a L at position Y7 of SEQ ID NO: 1, and a W at position P36 of SEQ ID NO: 1.

### Y7L_P36W_P90F

In some embodiments, a KOD polymerase variant comprises: a L at a position corresponding to Y7 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a F at position P90 of SEQ ID NO: 1. In some embodiments, a KOD polymerase variant comprises: a L at position Y7 of SEQ ID NO: 1, a W at position P36 of SEQ ID NO: 1, and a F at position P90 of SEQ ID NO: 1.

### Y7L_P36W_P90F_R97A

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, and an A at position R97 of SEQ ID NO: 1.

### Y7L_P36W_P90F_V93I

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, and an I at position V93 of SEQ ID NO: 1.

### Y7L_P36W_P90F_V93I_R97A

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7L_P36W_P90F_V93I_R97F

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### Y7L_P36W_P90F_V93I_R97W

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1.

### Y7L_P36W_P90F_V93R_R97A

In some embodiments, a KOD polymerase variant comprises: a L at a position corresponding to Y7 of SEQ ID NO: 1, a W at a position corresponding to P36 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a F at position P90 of SEQ ID NO: 1, a R at position V93 of SEQ ID NO: 1, and an A at position R97 of SEQ ID NO: 1.

### Y7L_P36W_P90I

In some embodiments, a KOD polymerase variant comprises: a L at a position corresponding to Y7 of SEQ ID NO: 1, a W at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and an I at position P90 of SEQ ID NO: 1. In some embodiments, a KOD polymerase variant comprises: a L at position Y7 of SEQ ID NO: 1, a W at position P36 of SEQ ID NO: 1, and an I at position P90 of SEQ ID NO: 1.

### Y7L_P36W_P90I_R97A

In some embodiments, a KOD polymerase variant comprises: a L at a position corresponding to Y7 of SEQ ID NO: 1, a W at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, an I at position P90 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, an I at position P90 of SEQ ID NO: 1, and an A at position R97 of SEQ ID NO: 1.

### Y7L_P36W_P90I_V93I

In some embodiments, a KOD polymerase variant comprises: a L at a position corresponding to Y7 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, an I at position P90 of SEQ ID NO: 1, and an I at position V93 of SEQ ID NO: 1.

### Y7L_P36W_P90S_V93I

In some embodiments, a KOD polymerase variant comprises: a L at a position corresponding to Y7 of SEQ ID NO: 1, a W at a position corresponding to P36 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a S at position P90 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, a S at position P90 of SEQ ID NO: 1, and an I at position V93 of SEQ ID NO: 1.

### Y7L_P36W_P90W

In some embodiments, a KOD polymerase variant comprises: a L at a position corresponding to Y7 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a W at position P90 of SEQ ID NO: 1. In some embodiments, a KOD polymerase variant comprises: a L at position Y7 of SEQ ID NO: 1, a W at position P36 of SEQ ID NO: 1, and a W at position P90 of SEQ ID NO: 1.

### Y7L_P36W_P90W_R97A

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, and an A at position R97 of SEQ ID NO: 1.

### Y7L_P36W_P90W_R97F

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, and a F at position R97 of SEQ ID NO: 1.

### Y7L_P36W_P90W_V93I

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, and an I at position V93 of SEQ ID NO: 1.

### Y7L_P36W_P90W_V93I_R97A

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### Y7L_P36W_P90W_V93I_R97F

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### Y7L_P36W_P90W_V93I_R97H

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1.

### Y7L_P36W_P90W_V93I_R97W

In some embodiments, a KOD polymerase variant comprises: a L at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position P36 of SEQ ID NO: 1, a W at position P90 of SEQ ID NO: 1, an I at position V93 of SEQ ID NO: 1, and a W at position R97 of SEQ ID NO: 1.

### Y7L_P36W_P90Y

In some embodiments, a KOD polymerase variant comprises: a L at a position corresponding to Y7 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a Y at position P90 of SEQ ID NO: 1. In some embodiments, a KOD polymerase variant comprises: a L at position Y7 of SEQ ID NO: 1, a W at position P36 of SEQ ID NO: 1, and a Y at position P90 of SEQ ID NO: 1.

### Y7L_P36W_P90Y_R97A

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, and an A at position R97 of SEQ ID NO: 1.

### Y7L_P36W_P90Y_R97F

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, and a F at position R97 of SEQ ID NO: 1.

### Y7L_P36W_P90Y_R97H

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, and a H at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, and a H at position R97 of SEQ ID NO:1.

### Y7L_P36W_P90Y_R97W

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, and a W at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, and a W at position R97 of SEQ ID NO: 1.

### Y7L_P36W_P90Y_V93I

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, and an I at position V93 of SEQ ID NO: 1.

### Y7L_P36W_P90Y_V93I_R97F

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, an I at position V93 of SEQ ID NO: 1, and a F at position R97 of SEQ ID NO: 1.

### Y7L_P36W_P90Y_V93I_R97H

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7L_P36W_P90Y_V93I_R97W

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1.

### Y7L_P36W_P90Y_V93R_R97A

In some embodiments, a KOD polymerase variant comprises: a L at a position corresponding to Y7 of SEQ ID NO: 1, a W at a position corresponding to P36 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a Y at position P90 of SEQ ID NO: 1, a R at position V93 of SEQ ID NO: 1, and an A at position R97 of SEQ ID NO: 1.

### Y7L_P36W_R97A

In some embodiments, a KOD polymerase variant comprises: a L at a position corresponding to Y7 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, and an A at position R97 of SEQ ID NO: 1.

### Y7L_P36W_R97H

In some embodiments, a KOD polymerase variant comprises: a L at a position corresponding to Y7 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a H at position R97 of SEQ ID NO: 1. In some embodiments, a KOD polymerase variant comprises: a L at position Y7 of SEQ ID NO: 1, a W at position P36 of SEQ ID NO: 1, and a H at position R97 of SEQ ID NO: 1.

### Y7L_P36W_V93I

In some embodiments, a KOD polymerase variant comprises: a L at a position corresponding to Y7 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and an I at position V93 of SEQ ID NO: 1. In some embodiments, a KOD polymerase variant comprises: a L at position Y7 of SEQ ID NO: 1, a W at position P36 of SEQ ID NO: 1, and an I at position V93 of SEQ ID NO: 1.

### Y7L_P36W_V93I_R97A

In some embodiments, a KOD polymerase variant comprises: a L at a position corresponding to Y7 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, an I at position V93 of SEQ ID NO: 1, and an A at position R97 of SEQ ID NO: 1.

### Y7L_P36W_V93I_R97F

In some embodiments, a KOD polymerase variant comprises: a L at a position corresponding to Y7 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, an I at position V93 of SEQ ID NO: 1, and a F at position R97 of SEQ ID NO: 1.

### Y7L_P36W_V93I_R97H

In some embodiments, a KOD polymerase variant comprises: a L at a position corresponding to Y7 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position P36 of SEQ ID NO: 1, an I at position V93 of SEQ ID NO: 1, and a H at position R97 of SEQ ID NO: 1.

### Y7L_P36W_V93K

In some embodiments, a KOD polymerase variant comprises: a L at a position corresponding to Y7 of SEQ ID NO: 1, a W at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a K at position V93 of SEQ ID NO: 1. In some embodiments, a KOD polymerase variant comprises: a L at position Y7 of SEQ ID NO: 1, a W at position P36 of SEQ ID NO: 1, and a K at position V93 of SEQ ID NO: 1.

### Y7L_P36W_V93Q

In some embodiments, a KOD polymerase variant comprises: a L at a position corresponding to Y7 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a Q at position V93 of SEQ ID NO: 1. In some embodiments, a KOD polymerase variant comprises: a L at position Y7 of SEQ ID NO: 1, a W at position P36 of SEQ ID NO: 1, and a Q at position V93 of SEQ ID NO: 1.

### Y7L_P36W_V93R

In some embodiments, a KOD polymerase variant comprises: a L at a position corresponding to Y7 of SEQ ID NO: 1, a W at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, and a R at position V93 of SEQ ID NO: 1. In some embodiments, a KOD polymerase variant comprises: a L at position Y7 of SEQ ID NO: 1, a W at position P36 of SEQ ID NO: 1, and a R at position V93 of SEQ ID NO: 1.

### Y7L_P36W_V93R_R97A

In some embodiments, a KOD polymerase variant comprises: a L at a position corresponding to Y7 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a W at position P36 of SEQ ID NO: 1, a R at position V93 of SEQ ID NO: 1, and an A at position R97 of SEQ ID NO: 1.

### Y7L_P36W_V93R_R97F

In some embodiments, a KOD polymerase variant comprises: a L at a position corresponding to Y7 of SEQ ID NO: 1, a W at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P36 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a W at position P36 of SEQ ID NO: 1, a R at position V93 of SEQ ID NO: 1, and a F at position R97 of SEQ ID NO: 1.

### Y7L_P90F

In some embodiments, a KOD polymerase variant comprises: a L at a position corresponding to Y7 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a F at position P90 of SEQ ID NO: 1. In some embodiments, a KOD polymerase variant comprises: a L at position Y7 of SEQ ID NO: 1, and a F at position P90 of SEQ ID NO: 1.

### Y7L_P90F_R97A

In some embodiments, a KOD polymerase variant comprises: a L at a position corresponding to Y7 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and an A at position R97 of SEQ ID NO: 1. In some embodiments, a KOD polymerase variant comprises: a L at position Y7 of SEQ ID NO: 1, a F at position P90 of SEQ ID NO: 1, and an A at position R97 of SEQ ID NO: 1.

### Y7L_P90F_R97F

In some embodiments, a KOD polymerase variant comprises: a L at a position corresponding to Y7 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a F at position R97 of SEQ ID NO: 1. In some embodiments, a KOD polymerase variant comprises: a L at position Y7 of SEQ ID NO: 1, a F at position P90 of SEQ ID NO: 1, and a F at position R97 of SEQ ID NO: 1.

### Y7L_P90F_R97H

In some embodiments, a KOD polymerase variant comprises: a L at a position corresponding to Y7 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a H at position R97 of SEQ ID NO: 1. In some embodiments, a KOD polymerase variant comprises: a L at position Y7 of SEQ ID NO: 1, a F at position P90 of SEQ ID NO: 1, and a H at position R97 of SEQ ID NO: 1.

### Y7L_P90F_R97W

In some embodiments, a KOD polymerase variant comprises: a L at a position corresponding to Y7 of SEQ ID NO: 1, a F at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a W at position R97 of SEQ ID NO: 1. In some embodiments, a KOD polymerase variant comprises: a L at position Y7 of SEQ ID NO: 1, a F at position P90 of SEQ ID NO: 1, and a W at position R97 of SEQ ID NO: 1.

### Y7L_P90F_V93E_R97F

In some embodiments, a KOD polymerase variant comprises: a L at a position corresponding to Y7 of SEQ ID NO: 1, a F at a position corresponding to P90 of SEQ ID NO: 1, an E at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, an E at position V93 of SEQ ID NO: 1, and a F at position R97 of SEQ ID NO: 1. In some embodiments, a KOD polymerase variant comprises: a L at position Y7 of SEQ ID NO: 1, a F at position P90 of SEQ ID NO: 1, an E at position V93 of SEQ ID NO: 1, and a F at position R97 of SEQ ID NO: 1.

### Y7L_P90F_V93I

In some embodiments, a KOD polymerase variant comprises: a L at a position corresponding to Y7 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and an I at position V93 of SEQ ID NO: 1. In some embodiments, a KOD polymerase variant comprises: a L at position Y7 of SEQ ID NO: 1, a F at position P90 of SEQ ID NO: 1, and an I at position V93 of SEQ ID NO: 1.

### Y7L_P90F_V93I_R97A

In some embodiments, a KOD polymerase variant comprises: a L at a position corresponding to Y7 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a F at position P90 of SEQ ID NO: 1, an I at position V93 of SEQ ID NO: 1, and an A at position R97 of SEQ ID NO: 1.

### Y7L_P90F_V93I_R97F

In some embodiments, a KOD polymerase variant comprises: a L at a position corresponding to Y7 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a F at position P90 of SEQ ID NO: 1, an I at position V93 of SEQ ID NO: 1, and a F at position R97 of SEQ ID NO: 1.

### Y7L_P90F_V93I_R97H

In some embodiments, a KOD polymerase variant comprises: a L at a position corresponding to Y7 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a F at position P90 of SEQ ID NO: 1, an I at position V93 of SEQ ID NO: 1, and a H at position R97 of SEQ ID NO: 1.

### Y7L_P90F_V93I_R97W

In some embodiments, a KOD polymerase variant comprises: a L at a position corresponding to Y7 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a F at position P90 of SEQ ID NO: 1, an I at position V93 of SEQ ID NO: 1, and a W at position R97 of SEQ ID NO: 1.

### Y7L_P90F_V93K

In some embodiments, a KOD polymerase variant comprises: a L at a position corresponding to Y7 of SEQ ID NO: 1, a F at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a K at position V93 of SEQ ID NO: 1. In some embodiments, a KOD polymerase variant comprises: a L at position Y7 of SEQ ID NO: 1, a F at position P90 of SEQ ID NO: 1, and a K at position V93 of SEQ ID NO: 1.

### Y7L_P90F_V93K_R97A

In some embodiments, a KOD polymerase variant comprises: a L at a position corresponding to Y7 of SEQ ID NO: 1, a F at a position corresponding to P90 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, a K at position V93 of SEQ ID NO: 1, and an A at position R97 of SEQ ID NO: 1. In some embodiments, a KOD polymerase variant comprises: a L at position Y7 of SEQ ID NO: 1, a F at position P90 of SEQ ID NO: 1, a K at position V93 of SEQ ID NO: 1, and an A at position R97 of SEQ ID NO: 1.

### Y7L_P90F_V93K_R97F

In some embodiments, a KOD polymerase variant comprises: a L at a position corresponding to Y7 of SEQ ID NO: 1, a F at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, a K at position V93 of SEQ ID NO: 1, and a F at position R97 of SEQ ID NO: 1. In some embodiments, a KOD polymerase variant comprises: a L at position Y7 of SEQ ID NO: 1, a F at position P90 of SEQ ID NO: 1, a K at position V93 of SEQ ID NO: 1, and a F at position R97 of SEQ ID NO: 1.

### Y7L_P90F_V93K_R97F_R119Q

In some embodiments, a KOD polymerase variant comprises: a L at a position corresponding to Y7 of SEQ ID NO: 1, a F at a position corresponding to P90 of SEQ ID NO: 1, a K at a position corresponding to V93 of SEQ ID NO: 1, a F at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, a K at position V93 of SEQ ID NO: 1, a F at position R97 of SEQ ID NO: 1, and a Q at position R119 of SEQ ID NO: 1. In some embodiments, a KOD polymerase variant comprises: a L at position Y7 of SEQ ID NO: 1, a F at position P90 of SEQ ID NO: 1, a K at position V93 of SEQ ID NO: 1, a F at position R97 of SEQ ID NO: 1, and a Q at position R119 of SEQ ID NO: 1.

### Y7L_P90F_V93Q

In some embodiments, a KOD polymerase variant comprises: a L at a position corresponding to Y7 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a Q at position V93 of SEQ ID NO: 1. In some embodiments, a KOD polymerase variant comprises: a L at position Y7 of SEQ ID NO: 1, a F at position P90 of SEQ ID NO: 1, and a Q at position V93 of SEQ ID NO: 1.

### Y7L_P90F_V93Q_R97H

In some embodiments, a KOD polymerase variant comprises: a L at a position corresponding to Y7 of SEQ ID NO: 1, a F at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, a Q at position V93 of SEQ ID NO: 1, and a H at position R97 of SEQ ID NO: 1. In some embodiments, a KOD polymerase variant comprises: a L at position Y7 of SEQ ID NO: 1, a F at position P90 of SEQ ID NO: 1, a Q at position V93 of SEQ ID NO: 1, and a H at position R97 of SEQ ID NO: 1.

### Y7L_P90F_V93R

In some embodiments, a KOD polymerase variant comprises: a L at a position corresponding to Y7 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, and a R at position V93 of SEQ ID NO: 1. In some embodiments, a KOD polymerase variant comprises: a L at position Y7 of SEQ ID NO: 1, a F at position P90 of SEQ ID NO: 1, and a R at position V93 of SEQ ID NO: 1.

### Y7L_P90F_V93R_R97A

In some embodiments, a KOD polymerase variant comprises: a L at a position corresponding to Y7 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a F at position P90 of SEQ ID NO: 1, a R at position V93 of SEQ ID NO: 1, and an A at position R97 of SEQ ID NO:1.

### Y7L_P90I

In some embodiments, a KOD polymerase variant comprises: a L at a position corresponding to Y7 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and an I at position P90 of SEQ ID NO: 1. In some embodiments, a KOD polymerase variant comprises: a L at position Y7 of SEQ ID NO: 1, and an I at position P90 of SEQ ID NO: 1.

### Y7L_P90I_R97F

In some embodiments, a KOD polymerase variant comprises: a L at a position corresponding to Y7 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and a F at position R97 of SEQ ID NO: 1. In some embodiments, a KOD polymerase variant comprises: a L at position Y7 of SEQ ID NO: 1, an I at position P90 of SEQ ID NO: 1, and a F at position R97 of SEQ ID NO: 1.

### Y7L_P90I_R97H

In some embodiments, a KOD polymerase variant comprises: a L at a position corresponding to Y7 of SEQ ID NO: 1, an I at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and a H at position R97 of SEQ ID NO: 1. In some embodiments, a KOD polymerase variant comprises: a L at position Y7 of SEQ ID NO: 1, an I at position P90 of SEQ ID NO: 1, and a H at position R97 of SEQ ID NO: 1.

### Y7L_P90I_R97W

In some embodiments, a KOD polymerase variant comprises: a L at a position corresponding to Y7 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and a W at position R97 of SEQ ID NO: 1. In some embodiments, a KOD polymerase variant comprises: a L at position Y7 of SEQ ID NO: 1, an I at position P90 of SEQ ID NO: 1, and a W at position R97 of SEQ ID NO: 1.

### Y7L_P90I_V93I

In some embodiments, a KOD polymerase variant comprises: a L at a position corresponding to Y7 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and an I at position V93 of SEQ ID NO: 1. In some embodiments, a KOD polymerase variant comprises: a L at position Y7 of SEQ ID NO: 1, an I at position P90 of SEQ ID NO: 1, and an I at position V93 of SEQ ID NO: 1.

### Y7L_P90I_V93I_R97H

In some embodiments, a KOD polymerase variant comprises: a L at a position corresponding to Y7 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, an I at position P90 of SEQ ID NO: 1, an I at position V93 of SEQ ID NO: 1, and a H at position R97 of SEQ ID NO: 1.

### Y7L_P90I_V93I_R97W

In some embodiments, a KOD polymerase variant comprises: a L at a position corresponding to Y7 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, an I at position P90 of SEQ ID NO: 1, an I at position V93 of SEQ ID NO: 1, and a W at position R97 of SEQ ID NO: 1.

### Y7L_P90I_V93K_R97F

In some embodiments, a KOD polymerase variant comprises: a L at a position corresponding to Y7 of SEQ ID NO: 1, an I at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, a K at position V93 of SEQ ID NO: 1, and a F at position R97 of SEQ ID NO: 1. In some embodiments, a KOD polymerase variant comprises: a L at position Y7 of SEQ ID NO: 1, an I at position P90 of SEQ ID NO: 1, a K at position V93 of SEQ ID NO: 1, and a F at position R97 of SEQ ID NO: 1.

### Y7L_P90I_V93Q

In some embodiments, a KOD polymerase variant comprises: a L at a position corresponding to Y7 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and a Q at position V93 of SEQ ID NO: 1. In some embodiments, a KOD polymerase variant comprises: a L at position Y7 of SEQ ID NO: 1, an I at position P90 of SEQ ID NO: 1, and a Q at position V93 of SEQ ID NO: 1.

### Y7L_P90I_V93R

In some embodiments, a KOD polymerase variant comprises: a L at a position corresponding to Y7 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position P90 of SEQ ID NO: 1, and a R at position V93 of SEQ ID NO: 1. In some embodiments, a KOD polymerase variant comprises: a L at position Y7 of SEQ ID NO: 1, an I at position P90 of SEQ ID NO: 1, and a R at position V93 of SEQ ID NO: 1.

### Y7L_P90S

In some embodiments, a KOD polymerase variant comprises: a L at a position corresponding to Y7 of SEQ ID NO: 1 and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a S at position P90 of SEQ ID NO: 1. In some embodiments, a KOD polymerase variant comprises: a L at position Y7 of SEQ ID NO: 1, and a S at position P90 of SEQ ID NO: 1.

### Y7L_P90S_V93I_R97H

In some embodiments, a KOD polymerase variant comprises: a L at a position corresponding to Y7 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a S at position P90 of SEQ ID NO: 1, an I at position V93 of SEQ ID NO: 1, and a H at position R97 of SEQ ID NO: 1.

### Y7L_P90S_V93R_R97H

In some embodiments, a KOD polymerase variant comprises: a L at a position corresponding to Y7 of SEQ ID NO: 1, a S at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a S at position P90 of SEQ ID NO: 1, a R at position V93 of SEQ ID NO: 1, and a H at position R97 of SEQ ID NO: 1. In some embodiments, a KOD polymerase variant comprises: a L at position Y7 of SEQ ID NO: 1, a S at position P90 of SEQ ID NO: 1, a R at position V93 of SEQ ID NO: 1, and a H at position R97 of SEQ ID NO: 1.

### Y7L_P90W_R97A

In some embodiments, a KOD polymerase variant comprises: a L at a position corresponding to Y7 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and an A at position R97 of SEQ ID NO: 1. In some embodiments, a KOD polymerase variant comprises: a L at position Y7 of SEQ ID NO: 1, a W at position P90 of SEQ ID NO: 1, and an A at position R97 of SEQ ID NO: 1.

### Y7L_P90W_R97F

In some embodiments, a KOD polymerase variant comprises: a L at a position corresponding to Y7 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and a F at position R97 of SEQ ID NO: 1. In some embodiments, a KOD polymerase variant comprises: a L at position Y7 of SEQ ID NO: 1, a W at position P90 of SEQ ID NO: 1, and a F at position R97 of SEQ ID NO: 1.

### Y7L_P90W_R97H

In some embodiments, a KOD polymerase variant comprises: a L at a position corresponding to Y7 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and a H at position R97 of SEQ ID NO: 1. In some embodiments, a KOD polymerase variant comprises: a L at position Y7 of SEQ ID NO: 1, a W at position P90 of SEQ ID NO: 1, and a H at position R97 of SEQ ID NO: 1.

### Y7L_P90W_R97W

In some embodiments, a KOD polymerase variant comprises: a L at a position corresponding to Y7 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and a W at position R97 of SEQ ID NO: 1. In some embodiments, a KOD polymerase variant comprises: a L at position Y7 of SEQ ID NO: 1, a W at position P90 of SEQ ID NO: 1, and a W at position R97 of SEQ ID NO: 1.

### Y7L_P90W_V93I

In some embodiments, a KOD polymerase variant comprises: a L at a position corresponding to Y7 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and an I at position V93 of SEQ ID NO: 1. In some embodiments, a KOD polymerase variant comprises: a L at position Y7 of SEQ ID NO: 1, a W at position P90 of SEQ ID NO: 1, and an I at position V93 of SEQ ID NO: 1.

### Y7L_P90W_V93I_R97A

In some embodiments, a KOD polymerase variant comprises: a L at a position corresponding to Y7 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a W at position P90 of SEQ ID NO: 1, an I at position V93 of SEQ ID NO: 1, and an A at position R97 of SEQ ID NO: 1.

### Y7L_P90W_V93I_R97F

In some embodiments, a KOD polymerase variant comprises: a L at a position corresponding to Y7 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a W at position P90 of SEQ ID NO: 1, an I at position V93 of SEQ ID NO: 1, and a F at position R97 of SEQ ID NO: 1.

### Y7L_P90W_V93I_R97H

In some embodiments, a KOD polymerase variant comprises: a L at a position corresponding to Y7 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a W at position P90 of SEQ ID NO: 1, an I at position V93 of SEQ ID NO: 1, and a H at position R97 of SEQ ID NO: 1.

### Y7L_P90W_V93I_R97W

In some embodiments, a KOD polymerase variant comprises: a L at a position corresponding to Y7 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, a W at position P90 of SEQ ID NO: 1, an I at position V93 of SEQ ID NO: 1, and a W at position R97 of SEQ ID NO: 1.

### Y7L_P90W_V93Q

In some embodiments, a KOD polymerase variant comprises: a L at a position corresponding to Y7 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and a Q at position V93 of SEQ ID NO: 1. In some embodiments, a KOD polymerase variant comprises: a L at position Y7 of SEQ ID NO: 1, a W at position P90 of SEQ ID NO: 1, and a Q at position V93 of SEQ ID NO: 1.

### Y7L_P90W_V93R

In some embodiments, a KOD polymerase variant comprises: a L at a position corresponding to Y7 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a W at position P90 of SEQ ID NO: 1, and a R at position V93 of SEQ ID NO: 1. In some embodiments, a KOD polymerase variant comprises: a L at position Y7 of SEQ ID NO: 1, a W at position P90 of SEQ ID NO: 1, and a R at position V93 of SEQ ID NO: 1.

### Y7L_P90Y

In some embodiments, a KOD polymerase variant comprises: a L at a position corresponding to Y7 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a Y at position P90 of SEQ ID NO: 1. In some embodiments, a KOD polymerase variant comprises: a L at position Y7 of SEQ ID NO: 1, and a Y at position P90 of SEQ ID NO: 1.

### Y7L_P90Y_R97F

In some embodiments, a KOD polymerase variant comprises: a L at a position corresponding to Y7 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, and a F at position R97 of SEQ ID NO: 1. In some embodiments, a KOD polymerase variant comprises: a L at position Y7 of SEQ ID NO: 1, a Y at position P90 of SEQ ID NO: 1, and a F at position R97 of SEQ ID NO: 1.

### Y7L_P90Y_R97H

In some embodiments, a KOD polymerase variant comprises: a L at a position corresponding to Y7 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, and a H at position R97 of SEQ ID NO: 1. In some embodiments, a KOD polymerase variant comprises: a L at position Y7 of SEQ ID NO: 1, a Y at position P90 of SEQ ID NO: 1, and a H at position R97 of SEQ ID NO: 1.

### Y7L_P90Y_R97W

In some embodiments, a KOD polymerase variant comprises: a L at a position corresponding to Y7 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, and a W at position R97 of SEQ ID NO: 1. In some embodiments, a KOD polymerase variant comprises: a L at position Y7 of SEQ ID NO: 1, a Y at position P90 of SEQ ID NO: 1, and a W at position R97 of SEQ ID NO: 1.

### Y7L_P90Y_V93I

In some embodiments, a KOD polymerase variant comprises: a L at a position corresponding to Y7 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, and an I at position V93 of SEQ ID NO: 1. In some embodiments, a KOD polymerase variant comprises: a L at position Y7 of SEQ ID NO: 1, a Y at position P90 of SEQ ID NO: 1, and an I at position V93 of SEQ ID NO: 1.

### Y7L_P90Y_V93I_R97A

In some embodiments, a KOD polymerase variant comprises: a L at a position corresponding to Y7 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a L at position Y7 of SEQ ID NO: 1, a Y at position P90 of SEQ ID NO: 1, an I at position V93 of SEQ ID NO: 1, and an A at position R97 of SEQ ID NO: 1.

### Y7L_P90Y_V93I_R97F

In some embodiments, a KOD polymerase variant comprises: a L at a position corresponding to Y7 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, a Y at position P90 of SEQ ID NO: 1, an I at position V93 of SEQ ID NO: 1, and a F at position R97 of SEQ ID NO: 1.

### Y7L_P90Y_V93I_R97H

In some embodiments, a KOD polymerase variant comprises: a L at a position corresponding to Y7 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, a Y at position P90 of SEQ ID NO: 1, an I at position V93 of SEQ ID NO: 1, and a H at position R97 of SEQ ID NO:1.

### Y7L_P90Y_V93R_R97A

In some embodiments, a KOD polymerase variant comprises: a L at a position corresponding to Y7 of SEQ ID NO: 1, a Y at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Y at position P90 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a Y at position P90 of SEQ ID NO: 1, a R at position V93 of SEQ ID NO: 1, and an A at position R97 of SEQ ID NO: 1.

### Y7L_R97F

In some embodiments, a KOD polymerase variant comprises: a L at a position corresponding to Y7 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a F at position R97 of SEQ ID NO: 1. In some embodiments, a KOD polymerase variant comprises: a L at position Y7 of SEQ ID NO: 1, and a F at position R97 of SEQ ID NO: 1.

### Y7L_R97H

In some embodiments, a KOD polymerase variant comprises: a L at a position corresponding to Y7 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a H at position R97 of SEQ ID NO: 1. In some embodiments, a KOD polymerase variant comprises: a L at position Y7 of SEQ ID NO: 1, and a H at position R97 of SEQ ID NO: 1.

### Y7L_R97W

In some embodiments, a KOD polymerase variant comprises: a L at a position corresponding to Y7 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a W at position R97 of SEQ ID NO: 1. In some embodiments, a KOD polymerase variant comprises: a L at position Y7 of SEQ ID NO: 1, and a W at position R97 of SEQ ID NO: 1.

### Y7L_V93I

In some embodiments, a KOD polymerase variant comprises: a L at a position corresponding to Y7 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and an I at position V93 of SEQ ID NO: 1. In some embodiments, a KOD polymerase variant comprises: a L at position Y7 of SEQ ID NO: 1, and an I at position V93 of SEQ ID NO: 1.

### Y7L_V93I_R97F

In some embodiments, a KOD polymerase variant comprises: a L at a position corresponding to Y7 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position V93 of SEQ ID NO: 1, and a F at position R97 of SEQ ID NO: 1. In some embodiments, a KOD polymerase variant comprises: a L at position Y7 of SEQ ID NO: 1, an I at position V93 of SEQ ID NO: 1, and a F at position R97 of SEQ ID NO: 1.

### Y7L_V93I_R97H

In some embodiments, a KOD polymerase variant comprises: a L at a position corresponding to Y7 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position V93 of SEQ ID NO: 1, and a H at position R97 of SEQ ID NO: 1. In some embodiments, a KOD polymerase variant comprises: a L at position Y7 of SEQ ID NO: 1, an I at position V93 of SEQ ID NO: 1, and a H at position R97 of SEQ ID NO: 1.

### Y7L_V93I_R97W

In some embodiments, a KOD polymerase variant comprises: a L at a position corresponding to Y7 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, an I at position V93 of SEQ ID NO: 1, and a W at position R97 of SEQ ID NO: 1. In some embodiments, a KOD polymerase variant comprises: a L at position Y7 of SEQ ID NO: 1, an I at position V93 of SEQ ID NO: 1, and a W at position R97 of SEQ ID NO: 1.

### Y7L_V93K

In some embodiments, a KOD polymerase variant comprises: a L at a position corresponding to Y7 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a K at position V93 of SEQ ID NO: 1. In some embodiments, a KOD polymerase variant comprises: a L at position Y7 of SEQ ID NO: 1, and a K at position V93 of SEQ ID NO: 1.

### Y7L_V93K_R97H

In some embodiments, a KOD polymerase variant comprises: a L at a position corresponding to Y7 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a K at position V93 of SEQ ID NO: 1, and a H at position R97 of SEQ ID NO: 1. In some embodiments, a KOD polymerase variant comprises: a L at position Y7 of SEQ ID NO: 1, a K at position V93 of SEQ ID NO: 1, and a H at position R97 of SEQ ID NO: 1.

### Y7L_V93Q

In some embodiments, a KOD polymerase variant comprises: a L at a position corresponding to Y7 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a Q at position V93 of SEQ ID NO: 1. In some embodiments, a KOD polymerase variant comprises: a L at position Y7 of SEQ ID NO: 1, and a Q at position V93 of SEQ ID NO: 1.

### Y7L_V93Q_R97A

In some embodiments, a KOD polymerase variant comprises: a L at a position corresponding to Y7 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Q at position V93 of SEQ ID NO: 1, and an A at position R97 of SEQ ID NO: 1. In some embodiments, a KOD polymerase variant comprises: a L at position Y7 of SEQ ID NO: 1, a Q at position V93 of SEQ ID NO: 1, and an A at position R97 of SEQ ID NO: 1.

### Y7L_V93Q_R97H

In some embodiments, a KOD polymerase variant comprises: a L at a position corresponding to Y7 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Q at position V93 of SEQ ID NO: 1, and a H at position R97 of SEQ ID NO: 1. In some embodiments, a KOD polymerase variant comprises: a L at position Y7 of SEQ ID NO: 1, a Q at position V93 of SEQ ID NO: 1, and a H at position R97 of SEQ ID NO: 1.

### Y7L_V93Q_R97W

In some embodiments, a KOD polymerase variant comprises: a L at a position corresponding to Y7 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a Q at position V93 of SEQ ID NO: 1, and a W at position R97 of SEQ ID NO: 1. In some embodiments, a KOD polymerase variant comprises: a L at position Y7 of SEQ ID NO: 1, a Q at position V93 of SEQ ID NO: 1, and a W at position R97 of SEQ ID NO: 1.

### Y7L_V93R

In some embodiments, a KOD polymerase variant comprises: a L at a position corresponding to Y7 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, and a R at position V93 of SEQ ID NO: 1. In some embodiments, a KOD polymerase variant comprises: a L at position Y7 of SEQ ID NO: 1, and a R at position V93 of SEQ ID NO: 1.

### Y7L_V93R_R97A

In some embodiments, a KOD polymerase variant comprises: a L at a position corresponding to Y7 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a R at position V93 of SEQ ID NO: 1, and an A at position R97 of SEQ ID NO: 1. In some embodiments, a KOD polymerase variant comprises: a L at position Y7 of SEQ ID NO: 1, a R at position V93 of SEQ ID NO: 1, and an A at position R97 of SEQ ID NO: 1.

### Y7L_V93R_R97F

In some embodiments, a KOD polymerase variant comprises: a L at a position corresponding to Y7 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a R at position V93 of SEQ ID NO: 1, and a F at position R97 of SEQ ID NO: 1. In some embodiments, a KOD polymerase variant comprises: a L at position Y7 of SEQ ID NO: 1, a R at position V93 of SEQ ID NO: 1, and a F at position R97 of SEQ ID NO: 1.

### Y7L_V93R_R97W

In some embodiments, a KOD polymerase variant comprises: a L at a position corresponding to Y7 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a L at position Y7 of SEQ ID NO: 1, a R at position V93 of SEQ ID NO: 1, and a W at position R97 of SEQ ID NO: 1. In some embodiments, a KOD polymerase variant comprises: a L at position Y7 of SEQ ID NO: 1, a R at position V93 of SEQ ID NO: 1, and a W at position R97 of SEQ ID NO: 1.

### Y7M

In some embodiments, a KOD polymerase variant comprises: a M at a position corresponding to Y7 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1. In some embodiments, a KOD polymerase variant comprises: a M at position Y7 of SEQ ID NO: 1.

### Y7M_I16F

In some embodiments, a KOD polymerase variant comprises: a M at a position corresponding to Y7 of SEQ ID NO: 1 and a F at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a F at position I16 of SEQ ID NO: 1. In some embodiments, a KOD polymerase variant comprises: a M at position Y7 of SEQ ID NO: 1, and a F at position I16 of SEQ ID NO: 1.

### Y7M_I16F_P36A

In some embodiments, a KOD polymerase variant comprises: a M at a position corresponding to Y7 of SEQ ID NO: 1, a F at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and an A at position P36 of SEQ ID NO: 1. In some embodiments, a KOD polymerase variant comprises: a M at position Y7 of SEQ ID NO: 1, a F at position I16 of SEQ ID NO: 1, and an A at position P36 of SEQ ID NO: 1.

### Y7M_I16F_P36A_P90F

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, and a F at position P90 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, and a F at position P90 of SEQ ID NO: 1.

### Y7M_I16F_P36A_P90F_V93I

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, a F at position P90 of SEQ ID NO: 1, and an I at position V93 of SEQ ID NO: 1.

### Y7M_I16F_P36A_P90W_V93I

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, a W at position P90 of SEQ ID NO: 1, and an I at position V93 of SEQ ID NO: 1.

### Y7M_I16F_P36A_P90Y_V93I_R97A

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, a Y at position P90 of SEQ ID NO: 1, an I at position V93 of SEQ ID NO: 1, and an A at position R97 of SEQ ID NO: 1.

### Y7M_I16F_P36A_R97A

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, and an A at position R97 of SEQ ID NO: 1.

### Y7M_I16F_P36A_V93I

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, and an I at position V93 of SEQ ID NO: 1.

### Y7M_I16F_P36A_V93I_R97A

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, an I at position V93 of SEQ ID NO: 1, and an A at position R97 of SEQ ID NO: 1.

### Y7M_I16F_P36A_V93I_R97F

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, an I at position V93 of SEQ ID NO: 1, and a F at position R97 of SEQ ID NO: 1.

### Y7M_I16F_P36A_V93Q

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an A at position P36 of SEQ ID NO: 1, and a Q at position V93 of SEQ ID NO: 1. In some embodiments, a KOD polymerase variant comprises: a M at position Y7 of SEQ ID NO: 1, a F at position I16 of SEQ ID NO: 1, an A at position P36 of SEQ ID NO: 1, and a Q at position V93 of SEQ ID NO: 1.

### Y7M_I16F_P36H

In some embodiments, a KOD polymerase variant comprises: a M at a position corresponding to Y7 of SEQ ID NO: 1, a F at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a H at position P36 of SEQ ID NO: 1. In some embodiments, a KOD polymerase variant comprises: a M at position Y7 of SEQ ID NO: 1, a F at position I16 of SEQ ID NO: 1, and a H at position P36 of SEQ ID NO: 1.

### Y7M_I16F_P36H_V93I

In some embodiments, a KOD polymerase variant comprises: a M at a position corresponding to Y7 of SEQ ID NO: 1, a F at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a H at position P36 of SEQ ID NO: 1, and an I at position V93 of SEQ ID NO: 1.

### Y7M_I16F_P36S_V93I

In some embodiments, a KOD polymerase variant comprises: a M at a position corresponding to Y7 of SEQ ID NO: 1, a F at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P36 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a S at position P36 of SEQ ID NO: 1, and an I at position V93 of SEQ ID NO: 1.

### Y7M_I16F_P36W

In some embodiments, a KOD polymerase variant comprises: a M at a position corresponding to Y7 of SEQ ID NO: 1, a F at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a W at position P36 of SEQ ID NO: 1. In some embodiments, a KOD polymerase variant comprises: a M at position Y7 of SEQ ID NO: 1, a F at position I16 of SEQ ID NO: 1, and a W at position P36 of SEQ ID NO: 1.

### Y7M_I16F_P36W_R97A

In some embodiments, a KOD polymerase variant comprises: a M at a position corresponding to Y7 of SEQ ID NO: 1, a F at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P36 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a W at position P36 of SEQ ID NO: 1, and an A at position R97 of SEQ ID NO: 1.

### Y7M_I16F_P90F

In some embodiments, a KOD polymerase variant comprises: a M at a position corresponding to Y7 of SEQ ID NO: 1, a F at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a F at position P90 of SEQ ID NO: 1. In some embodiments, a KOD polymerase variant comprises: a M at position Y7 of SEQ ID NO: 1, a F at position I16 of SEQ ID NO: 1, and a F at position P90 of SEQ ID NO: 1.

### Y7M_I16F_P90F_R97A

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, and an A at position R97 of SEQ ID NO: 1.

### Y7M_I16F_P90F_R97H

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, and a H at position R97 of SEQ ID NO: 1.

### Y7M_I16F_P90F_V93I

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, and an I at position V93 of SEQ ID NO: 1.

### Y7M_I16F_P90F_V93I_R97A

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, an I at position V93 of SEQ ID NO: 1, and an A at position R97 of SEQ ID NO: 1.

### Y7M_I16F_P90F_V93I_R97F

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, an I at position V93 of SEQ ID NO: 1, and a F at position R97 of SEQ ID NO: 1.

### Y7M_I16F_P90F_V93I_R97H

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, an I at position V93 of SEQ ID NO: 1, and a H at position R97 of SEQ ID NO: 1.

### Y7M_I16F_P90F_V93Q

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, and a Q at position V93 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, and a Q at position V93 of SEQ ID NO: 1.

### Y7M_I16F_P90F_V93Q_R97W

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, a Q at position V93 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, a Q at position V93 of SEQ ID NO: 1, and a W at position R97 of SEQ ID NO: 1.

### Y7M_I16F_P90F_V93R_R97A

In some embodiments, a KOD polymerase variant comprises: a M at a position corresponding to Y7 of SEQ ID NO: 1, a F at a position corresponding to I16 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a F at position P90 of SEQ ID NO: 1, a R at position V93 of SEQ ID NO: 1, and an A at position R97 of SEQ ID NO: 1.

### Y7M_I16F_P90I

In some embodiments, a KOD polymerase variant comprises: a M at a position corresponding to Y7 of SEQ ID NO: 1, a F at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and an I at position P90 of SEQ ID NO: 1. In some embodiments, a KOD polymerase variant comprises: a M at position Y7 of SEQ ID NO: 1, a F at position I16 of SEQ ID NO: 1, and an I at position P90 of SEQ ID NO: 1.

### Y7M_I16F_P90I_R97W

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, and a W at position R97 of SEQ ID NO: 1.

### Y7M_I16F_P90I_V93I

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, and an I at position V93 of SEQ ID NO: 1.

### Y7M_I16F_P90I_V93I_R97A

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, an I at position V93 of SEQ ID NO: 1, and an A at position R97 of SEQ ID NO: 1.

### Y7M_I16F_P90I_V93I_R97F

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, an I at position V93 of SEQ ID NO: 1, and a F at position R97 of SEQ ID NO: 1.

### Y7M_I16F_P90I_V93I_R97H

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, an I at position V93 of SEQ ID NO: 1, and a H at position R97 of SEQ ID NO: 1.

### Y7M_I16F_P90I_V93Q

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, and a Q at position V93 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, and a Q at position V93 of SEQ ID NO: 1.

### Y7M_I16F_P90I_V93Q_R97H

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, a Q at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, a Q at position V93 of SEQ ID NO: 1, and a H at position R97 of SEQ ID NO: 1.

### Y7M_I16F_P90I_V93R

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, and a R at position V93 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, and a R at position V93 of SEQ ID NO: 1.

### Y7M_I16F_P90I_V93R_R97F

In some embodiments, a KOD polymerase variant comprises: a M at a position corresponding to Y7 of SEQ ID NO: 1, a F at a position corresponding to I16 of SEQ ID NO: 1, an I at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position P90 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, an I at position P90 of SEQ ID NO: 1, a R at position V93 of SEQ ID NO: 1, and a F at position R97 of SEQ ID NO: 1.

### Y7M_I16F_P90S_R97A

In some embodiments, a KOD polymerase variant comprises: a M at a position corresponding to Y7 of SEQ ID NO: 1, a F at a position corresponding to I16 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P90 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a S at position P90 of SEQ ID NO: 1, and an A at position R97 of SEQ ID NO: 1.

### Y7M_I16F_P90S_R97H

In some embodiments, a KOD polymerase variant comprises: a M at a position corresponding to Y7 of SEQ ID NO: 1, a F at a position corresponding to I16 of SEQ ID NO: 1, a S at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P90 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a S at position P90 of SEQ ID NO: 1, and a H at position R97 of SEQ ID NO: 1.

### Y7M_I16F_P90S_V93I

In some embodiments, a KOD polymerase variant comprises: a M at a position corresponding to Y7 of SEQ ID NO: 1, a F at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a S at position P90 of SEQ ID NO: 1, and an I at position V93 of SEQ ID NO: 1.

### Y7M_I16F_P90S_V93I_R97A

In some embodiments, a KOD polymerase variant comprises: a M at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a S at position P90 of SEQ ID NO: 1, an I at position V93 of SEQ ID NO: 1, and an A at position R97 of SEQ ID NO: 1.

### Y7M_I16F_P90S_V93I_R97F

In some embodiments, a KOD polymerase variant comprises: a M at a position corresponding to Y7 of SEQ ID NO: 1, a F at a position corresponding to I16 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, a S at position P90 of SEQ ID NO: 1, an I at position V93 of SEQ ID NO: 1, and a F at position R97 of SEQ ID NO: 1.

### Y7M_I16F_P90W

In some embodiments, a KOD polymerase variant comprises: a M at a position corresponding to Y7 of SEQ ID NO: 1, a F at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a W at position P90 of SEQ ID NO: 1. In some embodiments, a KOD polymerase variant comprises: a M at position Y7 of SEQ ID NO: 1, a F at position I16 of SEQ ID NO: 1, and a W at position P90 of SEQ ID NO: 1.

### Y7M_I16F_P90W_R97A

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, and an A at position R97 of SEQ ID NO: 1.

### Y7M_I16F_P90W_V93I

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, and an I at position V93 of SEQ ID NO: 1.

### Y7M_I16F_P90W_V93I_R97A

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, an I at position V93 of SEQ ID NO: 1, and an A at position R97 of SEQ ID NO: 1.

### Y7M_I16F_P90W_V93I_R97F

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, an I at position V93 of SEQ ID NO: 1, and a F at position R97 of SEQ ID NO: 1.

### Y7M_I16F_P90W_V93I_R97H

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, an I at position V93 of SEQ ID NO: 1, and a H at position R97 of SEQ ID NO: 1.

### Y7M_I16F_P90W_V93Q

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, and a Q at position V93 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, and a Q at position V93 of SEQ ID NO: 1.

### Y7M_I16F_P90W_V93R_R97A

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, a R at position V93 of SEQ ID NO: 1, and an A at position R97 of SEQ ID NO: 1.

### Y7M_I16F_P90W_V93R_R97W

In some embodiments, a KOD polymerase variant comprises: a M at a position corresponding to Y7 of SEQ ID NO: 1, a F at a position corresponding to I16 of SEQ ID NO: 1, a W at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a W at position P90 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, a W at position P90 of SEQ ID NO: 1, a R at position V93 of SEQ ID NO: 1, and a W at position R97 of SEQ ID NO: 1.

### Y7M_I16F_P90Y

In some embodiments, a KOD polymerase variant comprises: a M at a position corresponding to Y7 of SEQ ID NO: 1, a F at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a Y at position P90 of SEQ ID NO: 1. In some embodiments, a KOD polymerase variant comprises: a M at position Y7 of SEQ ID NO: 1, a F at position I16 of SEQ ID NO: 1, and a Y at position P90 of SEQ ID NO: 1.

### Y7M_I16F_P90Y_R97A

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, and an A at position R97 of SEQ ID NO: 1.

### Y7M_I16F_P90Y_R97H

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, and a H at position R97 of SEQ ID NO: 1.

### Y7M_I16F_P90Y_V93I

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, and an I at position V93 of SEQ ID NO: 1.

### Y7M_I16F_P90Y_V93I_R97A

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, an I at position V93 of SEQ ID NO: 1, and an A at position R97 of SEQ ID NO: 1.

### Y7M_I16F_P90Y_V93I_R97F

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, an I at position V93 of SEQ ID NO: 1, and a F at position R97 of SEQ ID NO: 1.

### Y7M_I16F_P90Y_V93I_R97H

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, an I at position V93 of SEQ ID NO: 1, and a H at position R97 of SEQ ID NO: 1.

### Y7M_I16F_P90Y_V93I_R97W

In some embodiments, a KOD polymerase variant comprises: a M at a position corresponding to Y7 of SEQ ID NO: 1, a F at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, a Y at position P90 of SEQ ID NO: 1, an I at position V93 of SEQ ID NO: 1, and a W at position R97 of SEQ ID NO: 1.

### Y7M_I16F_R97A

In some embodiments, a KOD polymerase variant comprises: a M at a position corresponding to Y7 of SEQ ID NO: 1, a F at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, and an A at position R97 of SEQ ID NO: 1.

### Y7M_I16F_R97F

In some embodiments, a KOD polymerase variant comprises: a M at a position corresponding to Y7 of SEQ ID NO: 1, a F at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, and a F at position R97 of SEQ ID NO: 1.

### Y7M_I16F_R97H

In some embodiments, a KOD polymerase variant comprises: a M at a position corresponding to Y7 of SEQ ID NO: 1, a F at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, and a H at position R97 of SEQ ID NO: 1.

### Y7M_I16F_R97W

In some embodiments, a KOD polymerase variant comprises: a M at a position corresponding to Y7 of SEQ ID NO: 1, a F at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, and a W at position R97 of SEQ ID NO: 1.

### Y7M_I16F_R119Q

In some embodiments, a KOD polymerase variant comprises: a M at a position corresponding to Y7 of SEQ ID NO: 1, a F at a position corresponding to I16 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a Q at position R119 of SEQ ID NO: 1. In some embodiments, a KOD polymerase variant comprises: a M at position Y7 of SEQ ID NO: 1, a F at position I16 of SEQ ID NO: 1, and a Q at position R119 of SEQ ID NO: 1.

### Y7M_I16F_V93I

In some embodiments, a KOD polymerase variant comprises: a M at a position corresponding to Y7 of SEQ ID NO: 1, a F at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and an I at position V93 of SEQ ID NO: 1. In some embodiments, a KOD polymerase variant comprises: a M at position Y7 of SEQ ID NO: 1, a F at position I16 of SEQ ID NO: 1, and an I at position V93 of SEQ ID NO: 1.

### Y7M_I16F_V93I_R97A

In some embodiments, a KOD polymerase variant comprises: a M at a position corresponding to Y7 of SEQ ID NO: 1, a F at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, an I at position V93 of SEQ ID NO: 1, and an A at position R97 of SEQ ID NO: 1.

### Y7M_I16F_V93I_R97F

In some embodiments, a KOD polymerase variant comprises: a M at a position corresponding to Y7 of SEQ ID NO: 1, a F at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, an I at position V93 of SEQ ID NO: 1, and a F at position R97 of SEQ ID NO: 1.

### Y7M_I16F_V93I_R97H

In some embodiments, a KOD polymerase variant comprises: a M at a position corresponding to Y7 of SEQ ID NO: 1, a F at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, an I at position V93 of SEQ ID NO: 1, and a H at position R97 of SEQ ID NO: 1.

### Y7M_I16F_V93I_R97W

In some embodiments, a KOD polymerase variant comprises: a M at a position corresponding to Y7 of SEQ ID NO: 1, a F at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, an I at position V93 of SEQ ID NO: 1, and a W at position R97 of SEQ ID NO: 1.

### Y7M_I16F_V93K

In some embodiments, a KOD polymerase variant comprises: a M at a position corresponding to Y7 of SEQ ID NO: 1, a F at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a K at position V93 of SEQ ID NO: 1. In some embodiments, a KOD polymerase variant comprises: a M at position Y7 of SEQ ID NO: 1, a F at position I16 of SEQ ID NO: 1, and a K at position V93 of SEQ ID NO: 1.

### Y7M_I16F_V93Q

In some embodiments, a KOD polymerase variant comprises: a M at a position corresponding to Y7 of SEQ ID NO: 1, a F at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a Q at position V93 of SEQ ID NO: 1. In some embodiments, a KOD polymerase variant comprises: a M at position Y7 of SEQ ID NO: 1, a F at position I16 of SEQ ID NO: 1, and a Q at position V93 of SEQ ID NO: 1.

### Y7M_I16F_V93Q_R97H

In some embodiments, a KOD polymerase variant comprises: a M at a position corresponding to Y7 of SEQ ID NO: 1, a F at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a Q at position V93 of SEQ ID NO: 1, and a H at position R97 of SEQ ID NO: 1. In some embodiments, a KOD polymerase variant comprises: a M at position Y7 of SEQ ID NO: 1, a F at position I16 of SEQ ID NO: 1, a Q at position V93 of SEQ ID NO: 1, and a H at position R97 of SEQ ID NO: 1.

### Y7M_I16F_V93R

In some embodiments, a KOD polymerase variant comprises: a M at a position corresponding to Y7 of SEQ ID NO: 1, a F at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, and a R at position V93 of SEQ ID NO: 1. In some embodiments, a KOD polymerase variant comprises: a M at position Y7 of SEQ ID NO: 1, a F at position I16 of SEQ ID NO: 1, and a R at position V93 of SEQ ID NO: 1.

### Y7M_I16F_V93R_R97A

In some embodiments, a KOD polymerase variant comprises: a M at a position corresponding to Y7 of SEQ ID NO: 1, a F at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a F at position I16 of SEQ ID NO: 1, a R at position V93 of SEQ ID NO: 1, and an A at position R97 of SEQ ID NO: 1.

### Y7M_I16F_V93R_R97F

In some embodiments, a KOD polymerase variant comprises: a M at a position corresponding to Y7 of SEQ ID NO: 1, a F at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a F at position I16 of SEQ ID NO: 1, a R at position V93 of SEQ ID NO: 1, and a F at position R97 of SEQ ID NO: 1.

### Y7M_I16F_V93R_R97W

In some embodiments, a KOD polymerase variant comprises: a M at a position corresponding to Y7 of SEQ ID NO: 1, a F at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position I16 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a F at position I16 of SEQ ID NO: 1, a R at position V93 of SEQ ID NO: 1, and a W at position R97 of SEQ ID NO: 1.

### Y7M_I16H_P36H_P90W

In some embodiments, a KOD polymerase variant comprises: a M at a position corresponding to Y7 of SEQ ID NO: 1, a H at a position corresponding to I16 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position I16 of SEQ ID NO: 1, a H at position P36 of SEQ ID NO: 1, and a W at position P90 of SEQ ID NO: 1. In some embodiments, a KOD polymerase variant comprises: a M at position Y7 of SEQ ID NO: 1, a H at position I16 of SEQ ID NO: 1, a H at position P36 of SEQ ID NO: 1, and a W at position P90 of SEQ ID NO: 1.

### Y7M_I16H_P36H_V93I

In some embodiments, a KOD polymerase variant comprises: a M at a position corresponding to Y7 of SEQ ID NO: 1, a H at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position I16 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a H at position I16 of SEQ ID NO: 1, a H at position P36 of SEQ ID NO: 1, and an I at position V93 of SEQ ID NO: 1.

### Y7M_I16H_P36H_V93R

In some embodiments, a KOD polymerase variant comprises: a M at a position corresponding to Y7 of SEQ ID NO: 1, a H at a position corresponding to I16 of SEQ ID NO: 1, a H at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position I16 of SEQ ID NO: 1, a H at position P36 of SEQ ID NO: 1, and a R at position V93 of SEQ ID NO: 1. In some embodiments, a KOD polymerase variant comprises: a M at position Y7 of SEQ ID NO: 1, a H at position I16 of SEQ ID NO: 1, a H at position P36 of SEQ ID NO: 1, and a R at position V93 of SEQ ID NO: 1.

### Y7M_I16H_P90W_V93I

In some embodiments, a KOD polymerase variant comprises: a M at a position corresponding to Y7 of SEQ ID NO: 1, a H at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position I16 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a H at position I16 of SEQ ID NO: 1, a W at position P90 of SEQ ID NO: 1, and an I at position V93 of SEQ ID NO: 1.

### Y7M_I16L

In some embodiments, a KOD polymerase variant comprises: a M at a position corresponding to Y7 of SEQ ID NO: 1 and a L at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a L at position I16 of SEQ ID NO: 1. In some embodiments, a KOD polymerase variant comprises: a M at position Y7 of SEQ ID NO: 1, and a L at position I16 of SEQ ID NO: 1.

### Y7M_I16L_P36A

In some embodiments, a KOD polymerase variant comprises: a M at a position corresponding to Y7 of SEQ ID NO: 1, a L at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and an A at position P36 of SEQ ID NO: 1. In some embodiments, a KOD polymerase variant comprises: a M at position Y7 of SEQ ID NO: 1, a L at position I16 of SEQ ID NO: 1, and an A at position P36 of SEQ ID NO: 1.

### Y7M_I16L_P36A_P90F

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a F at position P90 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a F at position P90 of SEQ ID NO: 1.

### Y7M_I16L_P36A_P90F_V93I

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a F at position P90 of SEQ ID NO: 1, and an I at position V93 of SEQ ID NO: 1.

### Y7M_I16L_P36A_P90F_V93I_R97H

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7M_I16L_P36A_P90I_R97F

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, an I at position P90 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, an I at position P90 of SEQ ID NO: 1, and a F at position R97 of SEQ ID NO: 1.

### Y7M_I16L_P36A_P90I_V93I_R97H

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, an I at position P90 of SEQ ID NO: 1, an I at position V93 of SEQ ID NO: 1, and a H at position R97 of SEQ ID NO: 1.

### Y7M_I16L_P36A_P90S_V93I_R97H

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a S at position P90 of SEQ ID NO: 1, an I at position V93 of SEQ ID NO: 1, and a H at position R97 of SEQ ID NO: 1.

### Y7M_I16L_P36A_P90W

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a W at position P90 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a W at position P90 of SEQ ID NO: 1.

### Y7M_I16L_P36A_P90W_V93I

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a W at position P90 of SEQ ID NO: 1, and an I at position V93 of SEQ ID NO: 1.

### Y7M_I16L_P36A_P90W_V93I_R97A

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO:1.

### Y7M_I16L_P36A_P90Y_V93I

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a Y at position P90 of SEQ ID NO: 1, and an I at position V93 of SEQ ID NO: 1.

### Y7M_I16L_P36A_R97F

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a F at position R97 of SEQ ID NO: 1.

### Y7M_I16L_P36A_R97H

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a H at position R97 of SEQ ID NO: 1.

### Y7M_I16L_P36A_R97W

In some embodiments, a KOD polymerase variant comprises: a M at a position corresponding to Y7 of SEQ ID NO: 1, a F at a position corresponding to I16 of SEQ ID NO: 1, an A at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a W at position R97 of SEQ ID NO: 1.

### Y7M_I16L_P36A_V93I

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and an I at position V93 of SEQ ID NO: 1.

### Y7M_I16L_P36A_V93I_R97A

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, an I at position V93 of SEQ ID NO: 1, and an A at position R97 of SEQ ID NO: 1.

### Y7M_I16L_P36A_V93I_R97F

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1.

### Y7M_I16L_P36A_V93I_R97H

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, an I at position V93 of SEQ ID NO: 1, and a H at position R97 of SEQ ID NO: 1.

### Y7M_I16L_P36A_V93Q

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a Q at position V93 of SEQ ID NO: 1.

### Y7M_I16L_P36A_V93Q_R97A

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a Q at position V93 of SEQ ID NO: 1, and an A at position R97 of SEQ ID NO: 1.

### Y7M_I16L_P36A_V93R

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, and a R at position V93 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, and a R at position V93 of SEQ ID NO: 1.

### Y7M_I16L_P36A_V93R_R97A

In some embodiments, a KOD polymerase variant comprises: a M at a position corresponding to Y7 of SEQ ID NO: 1, a L at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an A at position P36 of SEQ ID NO: 1, a R at position V93 of SEQ ID NO: 1, and an A at position R97 of SEQ ID NO: 1.

### Y7M_I16L_P36H

In some embodiments, a KOD polymerase variant comprises: a M at a position corresponding to Y7 of SEQ ID NO: 1, a L at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a H at position P36 of SEQ ID NO: 1. In some embodiments, a KOD polymerase variant comprises: a M at position Y7 of SEQ ID NO: 1, a L at position I16 of SEQ ID NO: 1, and a H at position P36 of SEQ ID NO: 1.

### Y7M_I16L_P36H_P90F_R97A

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, a F at position P90 of SEQ ID NO: 1, and an A at position R97 of SEQ ID NO: 1.

### Y7M_I16L_P36H_P90F_V93I

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, a F at position P90 of SEQ ID NO: 1, and an I at position V93 of SEQ ID NO: 1.

### Y7M_I16L_P36H_R97W

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, and a W at position R97 of SEQ ID NO: 1.

### Y7M_I16L_P36H_V93I

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, and an I at position V93 of SEQ ID NO: 1.

### Y7M_I16L_P36H_V93Q

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, and a Q at position V93 of SEQ ID NO: 1.

### Y7M_I16L_P36H_V93Q_R97F

In some embodiments, a KOD polymerase variant comprises: a M at a position corresponding to Y7 of SEQ ID NO: 1, a L at a position corresponding to I16 of SEQ ID NO: 1, a H at a position corresponding to P36 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a H at position P36 of SEQ ID NO: 1, a Q at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a H at position P36 of SEQ ID NO: 1, a Q at position V93 of SEQ ID NO: 1, and a F at position R97 of SEQ ID NO: 1.

### Y7M_I16L_P36R

In some embodiments, a KOD polymerase variant comprises: a M at a position corresponding to Y7 of SEQ ID NO: 1, a L at a position corresponding to I16 of SEQ ID NO: 1, and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a R at position P36 of SEQ ID NO: 1. In some embodiments, a KOD polymerase variant comprises: a M at position Y7 of SEQ ID NO: 1, a L at position I16 of SEQ ID NO: 1, and a R at position P36 of SEQ ID NO: 1.

### Y7M_I16L_P36S

In some embodiments, a KOD polymerase variant comprises: a M at a position corresponding to Y7 of SEQ ID NO: 1, a L at a position corresponding to I16 of SEQ ID NO: 1, and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a S at position P36 of SEQ ID NO: 1. In some embodiments, a KOD polymerase variant comprises: a M at position Y7 of SEQ ID NO: 1, a L at position I16 of SEQ ID NO: 1, and a S at position P36 of SEQ ID NO: 1.

### Y7M_I16L_P36S_P90F_V93I

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, a F at position P90 of SEQ ID NO: 1, and an I at position V93 of SEQ ID NO: 1.

### Y7M_I16L_P36S_P90W_V93I

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, a W at position P90 of SEQ ID NO: 1, and an I at position V93 of SEQ ID NO: 1.

### Y7M_I16L_P36S_P90Y_V93I

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, a Y at position P90 of SEQ ID NO: 1, and an I at position V93 of SEQ ID NO: 1.

### Y7M_I16L_P36S_R97A

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, and an A at position R97 of SEQ ID NO: 1.

### Y7M_I16L_P36S_V93I

In some embodiments, a KOD polymerase variant comprises: a M at a position corresponding to Y7 of SEQ ID NO: 1, a F at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, and an I at position V93 of SEQ ID NO: 1.

### Y7M_I16L_P36S_V93I_R97A

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, an I at position V93 of SEQ ID NO: 1, and an A at position R97 of SEQ ID NO: 1.

### Y7M_I16L_P36S_V93I_R97H

In some embodiments, a KOD polymerase variant comprises: a M at a position corresponding to Y7 of SEQ ID NO: 1, a L at a position corresponding to I16 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a S at position P36 of SEQ ID NO: 1, an I at position V93 of SEQ ID NO: 1, and a H at position R97 of SEQ ID NO: 1.

### Y7M_I16L_P36W

In some embodiments, a KOD polymerase variant comprises: a M at a position corresponding to Y7 of SEQ ID NO: 1, a L at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a W at position P36 of SEQ ID NO: 1. In some embodiments, a KOD polymerase variant comprises: a M at position Y7 of SEQ ID NO: 1, a L at position I16 of SEQ ID NO: 1, and a W at position P36 of SEQ ID NO: 1.

### Y7M_I16L_P36W_P90F

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a F at position P90 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a F at position P90 of SEQ ID NO: 1.

### Y7M_I16L_P36W_P90F_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a F at position P90 of SEQ ID NO: 1, and an I at position V93 of SEQ ID NO: 1.

### Y7M_I16L_P36W_P90F_V93I_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7M_I16L_P36W_P90I_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, an I at position P90 of SEQ ID NO: 1, and an I at position V93 of SEQ ID NO: 1.

### Y7M_I16L_P36W_P90W

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a W at position P90 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a W at position P90 of SEQ ID NO: 1.

### Y7M_I16L_P36W_P90W_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a W at position P90 of SEQ ID NO: 1, and an A at position R97 of SEQ ID NO: 1.

### Y7M_I16L_P36W_P90W_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a W at position P90 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a W at position P90 of SEQ ID NO: 1, and a H at position R97 of SEQ ID NO: 1.

### Y7M_I16L_P36W_P90W_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a W at position P90 of SEQ ID NO: 1, and an I at position V93 of SEQ ID NO: 1.

### Y7M_I16L_P36W_P90Y

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a Y at position P90 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a Y at position P90 of SEQ ID NO: 1.

### Y7M_I16L_P36W_P90Y_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a Y at position P90 of SEQ ID NO: 1, and an I at position V93 of SEQ ID NO: 1.

### Y7M_I16L_P36W_P90Y_V93I_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7M_I16L_P36W_P90Y_V93I_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7M_I16L_P36W_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and an A at position R97 of SEQ ID NO: 1.

### Y7M_I16L_P36W_R97F

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a F at position R97 of SEQ ID NO: 1.

### Y7M_I16L_P36W_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a H at position R97 of SEQ ID NO: 1.

### Y7M_I16L_P36W_R97W

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a W at position R97 of SEQ ID NO: 1.

### Y7M_I16L_P36W_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and an I at position V93 of SEQ ID NO: 1.

### Y7M_I16L_P36W_V93I_R97A

In some embodiments, a KOD polymerase variant comprises: a M at a position corresponding to Y7 of SEQ ID NO: 1, a F at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, an I at position V93 of SEQ ID NO: 1, and an A at position R97 of SEQ ID NO: 1.

### Y7M_I16L_P36W_V93I_R97F

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, an I at position V93 of SEQ ID NO: 1, and a F at position R97 of SEQ ID NO: 1.

### Y7M_I16L_P36W_V93I_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, an I at position V93 of SEQ ID NO: 1, and a H at position R97 of SEQ ID NO: 1.

### Y7M_I16L_P36W_V93I_R97W

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, an I at position V93 of SEQ ID NO: 1, and a W at position R97 of SEQ ID NO: 1.

### Y7M_I16L_P36W_V93Q

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, and a Q at position V93 of SEQ ID NO: 1.

### Y7M_I16L_P36W_V93Q_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a Q at position V93 of SEQ ID NO: 1, and an A at position R97 of SEQ ID NO: 1.

### Y7M_I16L_P36W_V93R_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P36 of SEQ ID NO: 1, a R at position V93 of SEQ ID NO: 1, and an A at position R97 of SEQ ID NO: 1.

### Y7M_I16L_P90F

In some embodiments, a KOD polymerase variant comprises: a M at a position corresponding to Y7 of SEQ ID NO: 1, a L at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a F at position P90 of SEQ ID NO: 1. In some embodiments, a KOD polymerase variant comprises: a M at position Y7 of SEQ ID NO: 1, a L at position I16 of SEQ ID NO: 1, and a F at position P90 of SEQ ID NO: 1.

### Y7M_I16L_P90F_R97A

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and an A at position R97 of SEQ ID NO: 1.

### Y7M_I16L_P90F_R97F

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and a F at position R97 of SEQ ID NO: 1.

### Y7M_I16L_P90F_R97H

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and a H at position R97 of SEQ ID NO: 1.

### Y7M_I16L_P90F_R119Q

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and a Q at position R119 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and a Q at position R119 of SEQ ID NO: 1.

### Y7M_I16L_P90F_V93I

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and an I at position V93 of SEQ ID NO: 1.

### Y7M_I16L_P90F_V93I_R97A

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, an I at position V93 of SEQ ID NO: 1, and an A at position R97 of SEQ ID NO: 1.

### Y7M_I16L_P90F_V93I_R97F

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, an I at position V93 of SEQ ID NO: 1, and a F at position R97 of SEQ ID NO: 1.

### Y7M_I16L_P90F_V93I_R97H

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, an I at position V93 of SEQ ID NO: 1, and a H at position R97 of SEQ ID NO: 1.

### Y7M_I16L_P90F_V93K_R119Q

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, a K at a position corresponding to V93 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, a K at position V93 of SEQ ID NO: 1, and a Q at position R119 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, a K at position V93 of SEQ ID NO: 1, and a Q at position R119 of SEQ ID NO: 1.

### Y7M_I16L_P90F_V93Q

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, and a Q at position V93 of SEQ ID NO: 1.

### Y7M_I16L_P90F_V93Q_R97A

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, a Q at position V93 of SEQ ID NO: 1, and an A at position R97 of SEQ ID NO: 1.

### Y7M_I16L_P90F_V93R_R97W

In some embodiments, a KOD polymerase variant comprises: a M at a position corresponding to Y7 of SEQ ID NO: 1, a L at a position corresponding to I16 of SEQ ID NO: 1, a F at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a F at position P90 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a F at position P90 of SEQ ID NO: 1, a R at position V93 of SEQ ID NO: 1, and a W at position R97 of SEQ ID NO: 1.

### Y7M_I16L_P90I

In some embodiments, a KOD polymerase variant comprises: a M at a position corresponding to Y7 of SEQ ID NO: 1, a L at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and an I at position P90 of SEQ ID NO: 1. In some embodiments, a KOD polymerase variant comprises: a M at position Y7 of SEQ ID NO: 1, a L at position I16 of SEQ ID NO: 1, and an I at position P90 of SEQ ID NO: 1.

### Y7M_I16L_P90I_R97F

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, and a F at position R97 of SEQ ID NO: 1.

### Y7M_I16L_P90I_V93I

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, and an I at position V93 of SEQ ID NO: 1.

### Y7M_I16L_P90I_V93I_R97A

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, an I at position V93 of SEQ ID NO: 1, and an A at position R97 of SEQ ID NO: 1.

### Y7M_I16L_P90I_V93I_R97H

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, an I at position V93 of SEQ ID NO: 1, and a H at position R97 of SEQ ID NO: 1.

### Y7M_I16L_P90I_V93Q

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, and a Q at position V93 of SEQ ID NO: 1.

### Y7M_I16L_P90I_V93R

In some embodiments, a KOD polymerase variant comprises: a M at a position corresponding to Y7 of SEQ ID NO: 1, a L at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position P90 of SEQ ID NO: 1, and a R at position V93 of SEQ ID NO: 1. In some embodiments, a KOD polymerase variant comprises: a M at position Y7 of SEQ ID NO: 1, a L at position I16 of SEQ ID NO: 1, an I at position P90 of SEQ ID NO: 1, and a R at position V93 of SEQ ID NO: 1.

### Y7M_I16L_P90S_R97A

In some embodiments, a KOD polymerase variant comprises: a M at a position corresponding to Y7 of SEQ ID NO: 1, a L at a position corresponding to I16 of SEQ ID NO: 1, a S at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a S at position P90 of SEQ ID NO: 1, and an A at position R97 of SEQ ID NO: 1.

### Y7M_I16L_P90S_V93I

In some embodiments, a KOD polymerase variant comprises: a M at a position corresponding to Y7 of SEQ ID NO: 1, a L at a position corresponding to I16 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a S at position P90 of SEQ ID NO: 1, and an I at position V93 of SEQ ID NO: 1.

### Y7M_I16L_P90S_V93I_R97A

In some embodiments, a KOD polymerase variant comprises: a M at a position corresponding to Y7 of SEQ ID NO: 1, a L at a position corresponding to I16 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a S at position P90 of SEQ ID NO: 1, an I at position V93 of SEQ ID NO: 1, and an A at position R97 of SEQ ID NO: 1.

### Y7M_I16L_P90W

In some embodiments, a KOD polymerase variant comprises: a M at a position corresponding to Y7 of SEQ ID NO: 1, a L at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a W at position P90 of SEQ ID NO: 1. In some embodiments, a KOD polymerase variant comprises: a M at position Y7 of SEQ ID NO: 1, a L at position I16 of SEQ ID NO: 1, and a W at position P90 of SEQ ID NO: 1.

### Y7M_I16L_P90W_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, and an A at position R97 of SEQ ID NO: 1.

### Y7M_I16L_P90W_R97F

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, and a F at position R97 of SEQ ID NO: 1.

### Y7M_I16L_P90W_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, and a H at position R97 of SEQ ID NO: 1.

### Y7M_I16L_P90W_V93I

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, and an I at position V93 of SEQ ID NO: 1.

### Y7M_I16L_P90W_V93I_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, an I at position V93 of SEQ ID NO: 1, and an A at position R97 of SEQ ID NO: 1.

### Y7M_I16L_P90W_V93I_R97F

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, an I at position V93 of SEQ ID NO: 1, and a F at position R97 of SEQ ID NO: 1.

### Y7M_I16L_P90W_V93I_R97H

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, an I at position V93 of SEQ ID NO: 1, and a H at position R97 of SEQ ID NO: 1.

### Y7M_I16L_P90W_V93I_R97W

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, an I at position V93 of SEQ ID NO: 1, and a W at position R97 of SEQ ID NO: 1.

### Y7M_I16L_P90W_V93Q

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, and a Q at position V93 of SEQ ID NO: 1.

### Y7M_I16L_P90W_V93Q_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, a Q at position V93 of SEQ ID NO: 1, and an A at position R97 of SEQ ID NO: 1.

### Y7M_I16L_P90W_V93Q_R97W

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, a Q at position V93 of SEQ ID NO: 1, and a W at position R97 of SEQ ID NO: 1.

### Y7M_I16L_P90W_V93R_R97A

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, a R at position V93 of SEQ ID NO: 1, and an A at position R97 of SEQ ID NO: 1.

### Y7M_I16L_P90W_V93R_R97F

In some embodiments, a KOD polymerase variant comprises: a M at a position corresponding to Y7 of SEQ ID NO: 1, a L at a position corresponding to I16 of SEQ ID NO: 1, a W at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a W at position P90 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a W at position P90 of SEQ ID NO: 1, a R at position V93 of SEQ ID NO: 1, and a F at position R97 of SEQ ID NO: 1.

### Y7M_I16L_P90Y

In some embodiments, a KOD polymerase variant comprises: a M at a position corresponding to Y7 of SEQ ID NO: 1, a L at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a Y at position P90 of SEQ ID NO: 1. In some embodiments, a KOD polymerase variant comprises: a M at position Y7 of SEQ ID NO: 1, a L at position I16 of SEQ ID NO: 1, and a Y at position P90 of SEQ ID NO: 1.

### Y7M_I16L_P90Y_R97A

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, and an A at position R97 of SEQ ID NO: 1.

### Y7M_I16L_P90Y_R97F

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, and a F at position R97 of SEQ ID NO: 1.

### Y7M_I16L_P90Y_R97H

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, and a H at position R97 of SEQ ID NO: 1.

### Y7M_I16L_P90Y_V93I

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, and an I at position V93 of SEQ ID NO: 1.

### Y7M_I16L_P90Y_V93I_R97A

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, an I at position V93 of SEQ ID NO: 1, and an A at position R97 of SEQ ID NO: 1.

### Y7M_I16L_P90Y_V93I_R97F

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, an I at position V93 of SEQ ID NO: 1, and a F at position R97 of SEQ ID NO: 1.

### Y7M_I16L_P90Y_V93I_R97H

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, an I at position V93 of SEQ ID NO: 1, and a H at position R97 of SEQ ID NO: 1.

### Y7M_I16L_P90Y_V93I_R97W

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, an I at position V93 of SEQ ID NO: 1, and a W at position R97 of SEQ ID NO: 1.

### Y7M_I16L_P90Y_V93Q

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, and a Q at position V93 of SEQ ID NO: 1.

### Y7M_I16L_P90Y_V93Q_R97A

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, a Q at position V93 of SEQ ID NO: 1, and an A at position R97 of SEQ ID NO: 1.

### Y7M_I16L_P90Y_V93R_R97W

In some embodiments, a KOD polymerase variant comprises: a M at a position corresponding to Y7 of SEQ ID NO: 1, a L at a position corresponding to I16 of SEQ ID NO: 1, a Y at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Y at position P90 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a Y at position P90 of SEQ ID NO: 1, a R at position V93 of SEQ ID NO: 1, and a W at position R97 of SEQ ID NO: 1.

### Y7M_I16L_R97A

In some embodiments, a KOD polymerase variant comprises: a M at a position corresponding to Y7 of SEQ ID NO: 1, a L at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, and an A at position R97 of SEQ ID NO: 1.

### Y7M_I16L_R97A_R119M

In some embodiments, a KOD polymerase variant comprises: a M at a position corresponding to Y7 of SEQ ID NO: 1, a L at a position corresponding to I16 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an A at position R97 of SEQ ID NO: 1, and a M at position R119 of SEQ ID NO: 1. In some embodiments, a KOD polymerase variant comprises: a M at position Y7 of SEQ ID NO: 1, a L at position I16 of SEQ ID NO: 1, an A at position R97 of SEQ ID NO: 1, and a M at position R119 of SEQ ID NO: 1.

### Y7M_I16L_R97F

In some embodiments, a KOD polymerase variant comprises: a M at a position corresponding to Y7 of SEQ ID NO: 1, a L at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, and a F at position R97 of SEQ ID NO: 1.

### Y7M_I16L_R97H

In some embodiments, a KOD polymerase variant comprises: a M at a position corresponding to Y7 of SEQ ID NO: 1, a L at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, and a H at position R97 of SEQ ID NO: 1.

### Y7M_I16L_R97W

In some embodiments, a KOD polymerase variant comprises: a M at a position corresponding to Y7 of SEQ ID NO: 1, a L at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, and a W at position R97 of SEQ ID NO: 1.

### Y7M_I16L_V93E

In some embodiments, a KOD polymerase variant comprises: a M at a position corresponding to Y7 of SEQ ID NO: 1, a L at a position corresponding to I16 of SEQ ID NO: 1, and an E at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and an E at position V93 of SEQ ID NO: 1. In some embodiments, a KOD polymerase variant comprises: a M at position Y7 of SEQ ID NO: 1, a L at position I16 of SEQ ID NO: 1, and an E at position V93 of SEQ ID NO: 1.

### Y7M_I16L_V93I

In some embodiments, a KOD polymerase variant comprises: a M at a position corresponding to Y7 of SEQ ID NO: 1, a L at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and an I at position V93 of SEQ ID NO: 1. In some embodiments, a KOD polymerase variant comprises: a M at position Y7 of SEQ ID NO: 1, a L at position I16 of SEQ ID NO: 1, and an I at position V93 of SEQ ID NO: 1.

### Y7M_I16L_V93I_R97A

In some embodiments, a KOD polymerase variant comprises: a M at a position corresponding to Y7 of SEQ ID NO: 1, a L at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, an I at position V93 of SEQ ID NO: 1, and an A at position R97 of SEQ ID NO: 1.

### Y7M_I16L_V93I_R97F

In some embodiments, a KOD polymerase variant comprises: a M at a position corresponding to Y7 of SEQ ID NO: 1, a L at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, an I at position V93 of SEQ ID NO: 1, and a F at position R97 of SEQ ID NO: 1.

### Y7M_I16L_V93I_R97H

In some embodiments, a KOD polymerase variant comprises: a M at a position corresponding to Y7 of SEQ ID NO: 1, a L at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, an I at position V93 of SEQ ID NO: 1, and a H at position R97 of SEQ ID NO: 1.

### Y7M_I16L_V93I_R97P

In some embodiments, a KOD polymerase variant comprises: a M at a position corresponding to Y7 of SEQ ID NO: 1, a L at a position corresponding to I16 of SEQ ID NO: 1, an I at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position V93 of SEQ ID NO: 1, and a P at position R97 of SEQ ID NO: 1. In some embodiments, a KOD polymerase variant comprises: a M at position Y7 of SEQ ID NO: 1, a L at position I16 of SEQ ID NO: 1, an I at position V93 of SEQ ID NO: 1, and a P at position R97 of SEQ ID NO: 1.

### Y7M_I16L_V93I_R97W

In some embodiments, a KOD polymerase variant comprises: a M at a position corresponding to Y7 of SEQ ID NO: 1, a L at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, an I at position V93 of SEQ ID NO: 1, and a W at position R97 of SEQ ID NO: 1.

### Y7M_I16L_V93K

In some embodiments, a KOD polymerase variant comprises: a M at a position corresponding to Y7 of SEQ ID NO: 1, a L at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a K at position V93 of SEQ ID NO: 1. In some embodiments, a KOD polymerase variant comprises: a M at position Y7 of SEQ ID NO: 1, a L at position I16 of SEQ ID NO: 1, and a K at position V93 of SEQ ID NO: 1.

### Y7M_I16L_V93K_R97A

In some embodiments, a KOD polymerase variant comprises: a M at a position corresponding to Y7 of SEQ ID NO: 1, a L at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a K at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a K at position V93 of SEQ ID NO: 1, and an A at position R97 of SEQ ID NO: 1.

### Y7M_I16L_V93K_R97F

In some embodiments, a KOD polymerase variant comprises: a M at a position corresponding to Y7 of SEQ ID NO: 1, a L at a position corresponding to I16 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a K at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a K at position V93 of SEQ ID NO: 1, and a F at position R97 of SEQ ID NO: 1.

### Y7M_I16L_V93Q

In some embodiments, a KOD polymerase variant comprises: a M at a position corresponding to Y7 of SEQ ID NO: 1, a L at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a Q at position V93 of SEQ ID NO: 1. In some embodiments, a KOD polymerase variant comprises: a M at position Y7 of SEQ ID NO: 1, a L at position I16 of SEQ ID NO: 1, and a Q at position V93 of SEQ ID NO: 1.

### Y7M_I16L_V93Q_R97A

In some embodiments, a KOD polymerase variant comprises: a M at a position corresponding to Y7 of SEQ ID NO: 1, a L at a position corresponding to I16 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a Q at position V93 of SEQ ID NO: 1, and an A at position R97 of SEQ ID NO: 1.

### Y7M_I16L_V93Q_R97H

In some embodiments, a KOD polymerase variant comprises: a M at a position corresponding to Y7 of SEQ ID NO: 1, a L at a position corresponding to I16 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Q at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a Q at position V93 of SEQ ID NO: 1, and a H at position R97 of SEQ ID NO: 1.

### Y7M_I16L_V93Q_R97W

In some embodiments, a KOD polymerase variant comprises: a M at a position corresponding to Y7 of SEQ ID NO: 1, a L at a position corresponding to I16 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a Q at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a Q at position V93 of SEQ ID NO: 1, and a W at position R97 of SEQ ID NO: 1.

### Y7M_I16L_V93R

In some embodiments, a KOD polymerase variant comprises: a M at a position corresponding to Y7 of SEQ ID NO: 1, a L at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, and a R at position V93 of SEQ ID NO: 1. In some embodiments, a KOD polymerase variant comprises: a M at position Y7 of SEQ ID NO: 1, a L at position I16 of SEQ ID NO: 1, and a R at position V93 of SEQ ID NO: 1.

### Y7M_I16L_V93R_R97A

In some embodiments, a KOD polymerase variant comprises: a M at a position corresponding to Y7 of SEQ ID NO: 1, a L at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a L at position I16 of SEQ ID NO: 1, a R at position V93 of SEQ ID NO: 1, and an A at position R97 of SEQ ID NO: 1.

### Y7M_I16L_V93R_R97A_R119Q

In some embodiments, a KOD polymerase variant comprises: a M at a position corresponding to Y7 of SEQ ID NO: 1, a L at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a M at position Y7 of SEQ ID NO: 1, a L at position I16 of SEQ ID NO: 1, a R at position V93 of SEQ ID NO: 1, an A at position R97 of SEQ ID NO: 1, and a Q at position R119 of SEQ ID NO: 1.

### Y7M_I16L_V93R_R97F

In some embodiments, a KOD polymerase variant comprises: a M at a position corresponding to Y7 of SEQ ID NO: 1, a L at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a L at position I16 of SEQ ID NO: 1, a R at position V93 of SEQ ID NO: 1, and a F at position R97 of SEQ ID NO: 1.

### Y7M_I16L_V93R_R97H

In some embodiments, a KOD polymerase variant comprises: a M at a position corresponding to Y7 of SEQ ID NO: 1, a L at a position corresponding to I16 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a R at position V93 of SEQ ID NO: 1, and a H at position R97 of SEQ ID NO: 1. In some embodiments, a KOD polymerase variant comprises: a M at position Y7 of SEQ ID NO: 1, a L at position I16 of SEQ ID NO: 1, a R at position V93 of SEQ ID NO: 1, and a H at position R97 of SEQ ID NO: 1.

### Y7M_I16L_V93R_R97W

In some embodiments, a KOD polymerase variant comprises: a M at a position corresponding to Y7 of SEQ ID NO: 1, a L at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a L at position I16 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a L at position I16 of SEQ ID NO: 1, a R at position V93 of SEQ ID NO: 1, and a W at position R97 of SEQ ID NO: 1.

### Y7M_I16M

In some embodiments, a KOD polymerase variant comprises: a M at a position corresponding to Y7 of SEQ ID NO: 1 and a M at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a M at position I16 of SEQ ID NO: 1. In some embodiments, a KOD polymerase variant comprises: a M at position Y7 of SEQ ID NO: 1, and a M at position I16 of SEQ ID NO: 1.

### Y7M_I16M_P36A

In some embodiments, a KOD polymerase variant comprises: a M at a position corresponding to Y7 of SEQ ID NO: 1, a M at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and an A at position P36 of SEQ ID NO: 1. In some embodiments, a KOD polymerase variant comprises: a M at position Y7 of SEQ ID NO: 1, a M at position I16 of SEQ ID NO: 1, and an A at position P36 of SEQ ID NO: 1.

### Y7M_I16M_P36A_P90W_V93I

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, a W at position P90 of SEQ ID NO: 1, and an I at position V93 of SEQ ID NO: 1.

### Y7M_I16M_P36A_P90Y_V93I

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, a Y at position P90 of SEQ ID NO: 1, and an I at position V93 of SEQ ID NO: 1.

### Y7M_I16M_P36A_R97H

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, and a H at position R97 of SEQ ID NO: 1.

### Y7M_I16M_P36A_V93I

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, and an I at position V93 of SEQ ID NO: 1.

### Y7M_I16M_P36A_V93I_R97A

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, an I at position V93 of SEQ ID NO: 1, and an A at position R97 of SEQ ID NO: 1.

### Y7M_I16M_P36A_V93I_R97F

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, an I at position V93 of SEQ ID NO: 1, and a F at position R97 of SEQ ID NO: 1.

### Y7M_I16M_P36A_V93R

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, and a R at position V93 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, and a R at position V93 of SEQ ID NO: 1.

### Y7M_I16M_P36A_V93R_R97A

In some embodiments, a KOD polymerase variant comprises: a M at a position corresponding to Y7 of SEQ ID NO: 1, a M at a position corresponding to I16 of SEQ ID NO: 1, an A at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, an A at position P36 of SEQ ID NO: 1, a R at position V93 of SEQ ID NO: 1, and an A at position R97 of SEQ ID NO: 1.

### Y7M_I16M_P36H_V93I

In some embodiments, a KOD polymerase variant comprises: a M at a position corresponding to Y7 of SEQ ID NO: 1, a M at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a H at position P36 of SEQ ID NO: 1, and an I at position V93 of SEQ ID NO: 1.

### Y7M_I16M_P36H_V93I_R97A

In some embodiments, a KOD polymerase variant comprises: a M at a position corresponding to Y7 of SEQ ID NO: 1, a M at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a H at position P36 of SEQ ID NO: 1, an I at position V93 of SEQ ID NO: 1, and an A at position R97 of SEQ ID NO: 1.

### Y7M_I16M_P36H_V93I_R97F

In some embodiments, a KOD polymerase variant comprises: a M at a position corresponding to Y7 of SEQ ID NO: 1, a M at a position corresponding to I16 of SEQ ID NO: 1, a H at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a H at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, a H at position P36 of SEQ ID NO: 1, an I at position V93 of SEQ ID NO: 1, and a F at position R97 of SEQ ID NO: 1.

### Y7M_I16M_P36S_R97A

In some embodiments, a KOD polymerase variant comprises: a M at a position corresponding to Y7 of SEQ ID NO: 1, a M at a position corresponding to I16 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a S at position P36 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a S at position P36 of SEQ ID NO: 1, and an A at position R97 of SEQ ID NO: 1.

### Y7M_I16M_P36S_V93I_R97A

In some embodiments, a KOD polymerase variant comprises: a M at a position corresponding to Y7 of SEQ ID NO: 1, a M at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a S at position P36 of SEQ ID NO: 1, an I at position V93 of SEQ ID NO: 1, and an A at position R97 of SEQ ID NO: 1.

### Y7M_I16M_P36S_V93I_R97F

In some embodiments, a KOD polymerase variant comprises: a M at a position corresponding to Y7 of SEQ ID NO: 1, a M at a position corresponding to I16 of SEQ ID NO: 1, a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a S at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, a S at position P36 of SEQ ID NO: 1, an I at position V93 of SEQ ID NO: 1, and a F at position R97 of SEQ ID NO: 1.

### Y7M_I16M_P36S_V93R_R97A

In some embodiments, a KOD polymerase variant comprises: a M at a position corresponding to Y7 of SEQ ID NO: 1, a M at a position corresponding to I16 of SEQ ID NO: 1, a S at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a S at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a S at position P36 of SEQ ID NO: 1, a R at position V93 of SEQ ID NO: 1, and an A at position R97 of SEQ ID NO: 1.

### Y7M_I16M_P36W

In some embodiments, a KOD polymerase variant comprises: a M at a position corresponding to Y7 of SEQ ID NO: 1, a M at a position corresponding to I16 of SEQ ID NO: 1, and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a W at position P36 of SEQ ID NO: 1. In some embodiments, a KOD polymerase variant comprises: a M at position Y7 of SEQ ID NO: 1, a M at position I16 of SEQ ID NO: 1, and a W at position P36 of SEQ ID NO: 1.

### Y7M_I16M_P36W_P90F

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, and a F at position P90 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, and a F at position P90 of SEQ ID NO: 1.

### Y7M_I16M_P36W_P90F_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a F at position P90 of SEQ ID NO: 1, and an I at position V93 of SEQ ID NO: 1.

### Y7M_I16M_P36W_P90I_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, an I at position P90 of SEQ ID NO: 1, and an I at position V93 of SEQ ID NO: 1.

### Y7M_I16M_P36W_P90W_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a W at position P90 of SEQ ID NO: 1, and an A at position R97 of SEQ ID NO: 1.

### Y7M_I16M_P36W_P90W_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a W at position P90 of SEQ ID NO: 1, and an I at position V93 of SEQ ID NO: 1.

### Y7M_I16M_P36W_P90W_V93I_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO:1.

### Y7M_I16M_P36W_P90W_V93I_R97F

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### Y7M_I16M_P36W_P90Y

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, and a Y at position P90 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, and a Y at position P90 of SEQ ID NO: 1.

### Y7M_I16M_P36W_P90Y_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a Y at position P90 of SEQ ID NO: 1, and an A at position R97 of SEQ ID NO: 1.

### Y7M_I16M_P36W_P90Y_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a Y at position P90 of SEQ ID NO: 1, and an I at position V93 of SEQ ID NO: 1.

### Y7M_I16M_P36W_P90Y_V93I_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7M_I16M_P36W_P90Y_V93I_R97H

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7M_I16M_P36W_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, and an I at position V93 of SEQ ID NO:1.

### Y7M_I16M_P36W_V93I_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, an I at position V93 of SEQ ID NO: 1, and an A at position R97 of SEQ ID NO: 1.

### Y7M_I16M_P36W_V93I_R97H

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, an I at position V93 of SEQ ID NO: 1, and a H at position R97 of SEQ ID NO: 1.

### Y7M_I16M_P36W_V93I_R97W

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, an I at position V93 of SEQ ID NO: 1, and a W at position R97 of SEQ ID NO: 1.

### Y7M_I16M_P36W_V93R_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P36 of SEQ ID NO: 1, a R at position V93 of SEQ ID NO: 1, and an A at position R97 of SEQ ID NO: 1.

### Y7M_I16M_P90F

In some embodiments, a KOD polymerase variant comprises: a M at a position corresponding to Y7 of SEQ ID NO: 1, a M at a position corresponding to I16 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a F at position P90 of SEQ ID NO: 1. In some embodiments, a KOD polymerase variant comprises: a M at position Y7 of SEQ ID NO: 1, a M at position I16 of SEQ ID NO: 1, and a F at position P90 of SEQ ID NO: 1.

### Y7M_I16M_P90F_R97A

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, and an A at position R97 of SEQ ID NO: 1.

### Y7M_I16M_P90F_V93I

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, and an I at position V93 of SEQ ID NO: 1.

### Y7M_I16M_P90F_V93I_R97A

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, an I at position V93 of SEQ ID NO: 1, and an A at position R97 of SEQ ID NO: 1.

### Y7M_I16M_P90F_V93I_R97F

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, an I at position V93 of SEQ ID NO: 1, and a F at position R97 of SEQ ID NO: 1.

### Y7M_I16M_P90F_V93I_R97H

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, an I at position V93 of SEQ ID NO: 1, and a H at position R97 of SEQ ID NO: 1.

### Y7M_I16M_P90F_V93Q_R97A

In some embodiments, a KOD polymerase variant comprises: a M at a position corresponding to Y7 of SEQ ID NO: 1, a M at a position corresponding to I16 of SEQ ID NO: 1, a F at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a F at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a F at position P90 of SEQ ID NO: 1, a Q at position V93 of SEQ ID NO: 1, and an A at position R97 of SEQ ID NO: 1.

### Y7M_I16M_P90I

In some embodiments, a KOD polymerase variant comprises: a M at a position corresponding to Y7 of SEQ ID NO: 1, a M at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and an I at position P90 of SEQ ID NO: 1. In some embodiments, a KOD polymerase variant comprises: a M at position Y7 of SEQ ID NO: 1, a M at position I16 of SEQ ID NO: 1, and an I at position P90 of SEQ ID NO: 1.

### Y7M_I16M_P90I_R97F

In some embodiments, a KOD polymerase variant comprises: a M at a position corresponding to Y7 of SEQ ID NO: 1, a M at a position corresponding to I16 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position P90 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, an I at position P90 of SEQ ID NO: 1, and a F at position R97 of SEQ ID NO: 1.

### Y7M_I16M_P90I_V93I

In some embodiments, a KOD polymerase variant comprises: a M at a position corresponding to Y7 of SEQ ID NO: 1, a M at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, an I at position P90 of SEQ ID NO: 1, and an I at position V93 of SEQ ID NO: 1.

### Y7M_I16M_P90I_V93I_R97A

In some embodiments, a KOD polymerase variant comprises: a M at a position corresponding to Y7 of SEQ ID NO: 1, a M at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, an I at position P90 of SEQ ID NO: 1, an I at position V93 of SEQ ID NO: 1, and an A at position R97 of SEQ ID NO: 1.

### Y7M_I16M_P90I_V93K_R97W

In some embodiments, a KOD polymerase variant comprises: a M at a position corresponding to Y7 of SEQ ID NO: 1, a M at a position corresponding to I16 of SEQ ID NO: 1, an I at a position corresponding to P90 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position P90 of SEQ ID NO: 1, a K at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, an I at position P90 of SEQ ID NO: 1, a K at position V93 of SEQ ID NO: 1, and a W at position R97 of SEQ ID NO: 1.

### Y7M_I16M_P90I_V93R

In some embodiments, a KOD polymerase variant comprises: a M at a position corresponding to Y7 of SEQ ID NO: 1, a M at a position corresponding to I16 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position P90 of SEQ ID NO: 1, and a R at position V93 of SEQ ID NO: 1. In some embodiments, a KOD polymerase variant comprises: a M at position Y7 of SEQ ID NO: 1, a M at position I16 of SEQ ID NO: 1, an I at position P90 of SEQ ID NO: 1, and a R at position V93 of SEQ ID NO: 1.

### Y7M_I16M_P90S

In some embodiments, a KOD polymerase variant comprises: a M at a position corresponding to Y7 of SEQ ID NO: 1, a M at a position corresponding to I16 of SEQ ID NO: 1, and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a S at position P90 of SEQ ID NO: 1. In some embodiments, a KOD polymerase variant comprises: a M at position Y7 of SEQ ID NO: 1, a M at position I16 of SEQ ID NO: 1, and a S at position P90 of SEQ ID NO: 1.

### Y7M_I16M_P90W

In some embodiments, a KOD polymerase variant comprises: a M at a position corresponding to Y7 of SEQ ID NO: 1, a M at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a W at position P90 of SEQ ID NO: 1. In some embodiments, a KOD polymerase variant comprises: a M at position Y7 of SEQ ID NO: 1, a M at position I16 of SEQ ID NO: 1, and a W at position P90 of SEQ ID NO: 1.

### Y7M_I16M_P90W_R97H

In some embodiments, a KOD polymerase variant comprises: a M at a position corresponding to Y7 of SEQ ID NO: 1, a M at a position corresponding to I16 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P90 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a W at position P90 of SEQ ID NO: 1, and a H at position R97 of SEQ ID NO: 1.

### Y7M_I16M_P90W_V93I

In some embodiments, a KOD polymerase variant comprises: a M at a position corresponding to Y7 of SEQ ID NO: 1, a M at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a W at position P90 of SEQ ID NO: 1, and an I at position V93 of SEQ ID NO: 1.

### Y7M_I16M_P90W_V93I_R97A

In some embodiments, a KOD polymerase variant comprises: a M at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1.

### Y7M_I16M_P90W_V93I_R97H

In some embodiments, a KOD polymerase variant comprises: a M at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a W at position P90 of SEQ ID NO: 1, an I at position V93 of SEQ ID NO: 1, and a H at position R97 of SEQ ID NO: 1.

### Y7M_I16M_P90W_V93I_R97W

In some embodiments, a KOD polymerase variant comprises: a M at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a W at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, a W at position P90 of SEQ ID NO: 1, an I at position V93 of SEQ ID NO: 1, and a W at position R97 of SEQ ID NO: 1.

### Y7M_I16M_P90Y

In some embodiments, a KOD polymerase variant comprises: a M at a position corresponding to Y7 of SEQ ID NO: 1, a M at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a Y at position P90 of SEQ ID NO: 1. In some embodiments, a KOD polymerase variant comprises: a M at position Y7 of SEQ ID NO: 1, a M at position I16 of SEQ ID NO: 1, and a Y at position P90 of SEQ ID NO: 1.

### Y7M_I16M_P90Y_R97A

In some embodiments, a KOD polymerase variant comprises: a M at a position corresponding to Y7 of SEQ ID NO: 1, a M at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a Y at position P90 of SEQ ID NO: 1, and an A at position R97 of SEQ ID NO: 1.

### Y7M_I16M_P90Y_V93I

In some embodiments, a KOD polymerase variant comprises: a M at a position corresponding to Y7 of SEQ ID NO: 1, a M at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, a Y at position P90 of SEQ ID NO: 1, and an I at position V93 of SEQ ID NO: 1.

### Y7M_I16M_P90Y_V93I_R97A

In some embodiments, a KOD polymerase variant comprises: a M at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a Y at position P90 of SEQ ID NO: 1, an I at position V93 of SEQ ID NO: 1, and an A at position R97 of SEQ ID NO: 1.

### Y7M_I16M_P90Y_V93I_R97H

In some embodiments, a KOD polymerase variant comprises: a M at a position corresponding to Y7 of SEQ ID NO: 1, a M at a position corresponding to I16 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a Y at position P90 of SEQ ID NO: 1, an I at position V93 of SEQ ID NO: 1, and a H at position R97 of SEQ ID NO: 1.

### Y7M_I16M_P90Y_V93Q_R97A

In some embodiments, a KOD polymerase variant comprises: a M at a position corresponding to Y7 of SEQ ID NO: 1, a M at a position corresponding to I16 of SEQ ID NO: 1, a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Y at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a Y at position P90 of SEQ ID NO: 1, a Q at position V93 of SEQ ID NO: 1, and an A at position R97 of SEQ ID NO: 1.

### Y7M_I16M_R97A

In some embodiments, a KOD polymerase variant comprises: a M at a position corresponding to Y7 of SEQ ID NO: 1, a M at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, and an A at position R97 of SEQ ID NO: 1.

### Y7M_I16M_R97F

In some embodiments, a KOD polymerase variant comprises: a M at a position corresponding to Y7 of SEQ ID NO: 1, a M at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, and a F at position R97 of SEQ ID NO: 1.

### Y7M_I16M_R97H

In some embodiments, a KOD polymerase variant comprises: a M at a position corresponding to Y7 of SEQ ID NO: 1, a M at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, and a H at position R97 of SEQ ID NO: 1.

### Y7M_I16M_R97W

In some embodiments, a KOD polymerase variant comprises: a M at a position corresponding to Y7 of SEQ ID NO: 1, a M at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, and a W at position R97 of SEQ ID NO: 1.

### Y7M_I16M_V93I

In some embodiments, a KOD polymerase variant comprises: a M at a position corresponding to Y7 of SEQ ID NO: 1, a M at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and an I at position V93 of SEQ ID NO: 1. In some embodiments, a KOD polymerase variant comprises: a M at position Y7 of SEQ ID NO: 1, a M at position I16 of SEQ ID NO: 1, and an I at position V93 of SEQ ID NO: 1.

### Y7M_I16M_V93I_R97A

In some embodiments, a KOD polymerase variant comprises: a M at a position corresponding to Y7 of SEQ ID NO: 1, a M at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, an I at position V93 of SEQ ID NO: 1, and an A at position R97 of SEQ ID NO: 1.

### Y7M_I16M_V93I_R97F

In some embodiments, a KOD polymerase variant comprises: a M at a position corresponding to Y7 of SEQ ID NO: 1, a M at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, an I at position V93 of SEQ ID NO: 1, and a F at position R97 of SEQ ID NO: 1.

### Y7M_I16M_V93I_R97H

In some embodiments, a KOD polymerase variant comprises: a M at a position corresponding to Y7 of SEQ ID NO: 1, a M at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, an I at position V93 of SEQ ID NO: 1, and a H at position R97 of SEQ ID NO: 1.

### Y7M_I16M_V93I_R97W

In some embodiments, a KOD polymerase variant comprises: a M at a position corresponding to Y7 of SEQ ID NO: 1, a M at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, an I at position V93 of SEQ ID NO: 1, and a W at position R97 of SEQ ID NO: 1.

### Y7M_I16M_V93K

In some embodiments, a KOD polymerase variant comprises: a M at a position corresponding to Y7 of SEQ ID NO: 1, a M at a position corresponding to I16 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a K at position V93 of SEQ ID NO: 1. In some embodiments, a KOD polymerase variant comprises: a M at position Y7 of SEQ ID NO: 1, a M at position I16 of SEQ ID NO: 1, and a K at position V93 of SEQ ID NO: 1.

### Y7M_I16M_V93K_R97A

In some embodiments, a KOD polymerase variant comprises: a M at a position corresponding to Y7 of SEQ ID NO: 1, a M at a position corresponding to I16 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a K at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a K at position V93 of SEQ ID NO: 1, and an A at position R97 of SEQ ID NO: 1.

### Y7M_I16M_V93K_R97H

In some embodiments, a KOD polymerase variant comprises: a M at a position corresponding to Y7 of SEQ ID NO: 1, a M at a position corresponding to I16 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a K at position V93 of SEQ ID NO: 1, and a H at position R97 of SEQ ID NO: 1. In some embodiments, a KOD polymerase variant comprises: a M at position Y7 of SEQ ID NO: 1, a M at position I16 of SEQ ID NO: 1, a K at position V93 of SEQ ID NO: 1, and a H at position R97 of SEQ ID NO: 1.

### Y7M_I16M_V93K_R97W

In some embodiments, a KOD polymerase variant comprises: a M at a position corresponding to Y7 of SEQ ID NO: 1, a M at a position corresponding to I16 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a K at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, a K at position V93 of SEQ ID NO: 1, and a W at position R97 of SEQ ID NO: 1.

### Y7M_I16M_V93Q_R97F

In some embodiments, a KOD polymerase variant comprises: a M at a position corresponding to Y7 of SEQ ID NO: 1, a M at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a Q at position V93 of SEQ ID NO: 1, and a F at position R97 of SEQ ID NO: 1. In some embodiments, a KOD polymerase variant comprises: a M at position Y7 of SEQ ID NO: 1, a M at position I16 of SEQ ID NO: 1, a Q at position V93 of SEQ ID NO: 1, and a F at position R97 of SEQ ID NO: 1.

### Y7M_I16M_V93R

In some embodiments, a KOD polymerase variant comprises: a M at a position corresponding to Y7 of SEQ ID NO: 1, a M at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, and a R at position V93 of SEQ ID NO: 1. In some embodiments, a KOD polymerase variant comprises: a M at position Y7 of SEQ ID NO: 1, a M at position I16 of SEQ ID NO: 1, and a R at position V93 of SEQ ID NO: 1.

### Y7M_I16M_V93R_R97A

In some embodiments, a KOD polymerase variant comprises: a M at a position corresponding to Y7 of SEQ ID NO: 1, a M at a position corresponding to I16 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a M at position I16 of SEQ ID NO: 1, a R at position V93 of SEQ ID NO: 1, and an A at position R97 of SEQ ID NO: 1.

### Y7M_I16M_V93R_R97A_R119M

In some embodiments, a KOD polymerase variant comprises: a M at a position corresponding to Y7 of SEQ ID NO: 1, a M at a position corresponding to I16 of SEQ ID NO: 1, a R at a position corresponding to V93 of SEQ ID NO: 1, an A at a position corresponding to R97 of SEQ ID NO: 1, and a M at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a R at position V93 of SEQ ID NO: 1, an A at position R97 of SEQ ID NO: 1, and a M at position R119 of SEQ ID NO: 1. In some embodiments, a KOD polymerase variant comprises: a M at position Y7 of SEQ ID NO: 1, a M at position I16 of SEQ ID NO: 1, a R at position V93 of SEQ ID NO: 1, an A at position R97 of SEQ ID NO: 1, and a M at position R119 of SEQ ID NO: 1.

### Y7M_I16M_V93R_R97A_R119Q

In some embodiments, a KOD polymerase variant comprises: a M at a position corresponding to Y7 of SEQ ID NO: 1, a M at a position corresponding to I16 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a M at position Y7 of SEQ ID NO: 1, a M at position I16 of SEQ ID NO: 1, a R at position V93 of SEQ ID NO: 1, an A at position R97 of SEQ ID NO: 1, and a Q at position R119 of SEQ ID NO: 1.

### Y7M_I16M_V93R_R97P

In some embodiments, a KOD polymerase variant comprises: a M at a position corresponding to Y7 of SEQ ID NO: 1, a M at a position corresponding to I16 of SEQ ID NO: 1, a R at a position corresponding to V93 of SEQ ID NO: 1, and a P at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a R at position V93 of SEQ ID NO: 1, and a P at position R97 of SEQ ID NO: 1. In some embodiments, a KOD polymerase variant comprises: a M at position Y7 of SEQ ID NO: 1, a M at position I16 of SEQ ID NO: 1, a R at position V93 of SEQ ID NO: 1, and a P at position R97 of SEQ ID NO: 1.

### Y7M_I16M_V93R_R97W

In some embodiments, a KOD polymerase variant comprises: a M at a position corresponding to Y7 of SEQ ID NO: 1, a M at a position corresponding to I16 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a M at position I16 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, a M at position I16 of SEQ ID NO: 1, a R at position V93 of SEQ ID NO: 1, and a W at position R97 of SEQ ID NO: 1.

### Y7M_I16W

In some embodiments, a KOD polymerase variant comprises: a M at a position corresponding to Y7 of SEQ ID NO: 1 and a W at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a W at position I16 of SEQ ID NO: 1. In some embodiments, a KOD polymerase variant comprises: a M at position Y7 of SEQ ID NO: 1, and a W at position I16 of SEQ ID NO: 1.

### Y7M_I16W_P36A

In some embodiments, a KOD polymerase variant comprises: a M at a position corresponding to Y7 of SEQ ID NO: 1, a W at a position corresponding to I16 of SEQ ID NO: 1, and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and an A at position P36 of SEQ ID NO: 1. In some embodiments, a KOD polymerase variant comprises: a M at position Y7 of SEQ ID NO: 1, a W at position I16 of SEQ ID NO: 1, and an A at position P36 of SEQ ID NO: 1.

### Y7M_I16W_P36A_V93I

In some embodiments, a KOD polymerase variant comprises: a M at a position corresponding to Y7 of SEQ ID NO: 1, a W at a position corresponding to I16 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an A at position P36 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, an A at position P36 of SEQ ID NO: 1, and an I at position V93 of SEQ ID NO: 1.

### Y7M_I16W_P36A_V93I_R97A

In some embodiments, a KOD polymerase variant comprises: a M at a position corresponding to Y7 of SEQ ID NO: 1, a W at a position corresponding to I16 of SEQ ID NO: 1, an A at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an A at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, an A at position P36 of SEQ ID NO: 1, an I at position V93 of SEQ ID NO: 1, and an A at position R97 of SEQ ID NO: 1.

### Y7M_I16W_P36H

In some embodiments, a KOD polymerase variant comprises: a M at a position corresponding to Y7 of SEQ ID NO: 1, a W at a position corresponding to I16 of SEQ ID NO: 1, and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a H at position P36 of SEQ ID NO: 1. In some embodiments, a KOD polymerase variant comprises: a M at position Y7 of SEQ ID NO: 1, a W at position I16 of SEQ ID NO: 1, and a H at position P36 of SEQ ID NO: 1.

### Y7M_I16W_P36H_V93I

In some embodiments, a KOD polymerase variant comprises: a M at a position corresponding to Y7 of SEQ ID NO: 1, a W at a position corresponding to I16 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, a H at position P36 of SEQ ID NO: 1, and an I at position V93 of SEQ ID NO: 1.

### Y7M_I16W_P36H_V93I_R97A

In some embodiments, a KOD polymerase variant comprises: a M at a position corresponding to Y7 of SEQ ID NO: 1, a W at a position corresponding to I16 of SEQ ID NO: 1, a H at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a H at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, a H at position P36 of SEQ ID NO: 1, an I at position V93 of SEQ ID NO: 1, and an A at position R97 of SEQ ID NO: 1.

### Y7M_I16W_P36S_V93I

In some embodiments, a KOD polymerase variant comprises: a M at a position corresponding to Y7 of SEQ ID NO: 1, a W at a position corresponding to I16 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a S at position P36 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, a S at position P36 of SEQ ID NO: 1, and an I at position V93 of SEQ ID NO: 1.

### Y7M_I16W_P36S_V93I_R97A

In some embodiments, a KOD polymerase variant comprises: a M at a position corresponding to Y7 of SEQ ID NO: 1, a W at a position corresponding to I16 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, a S at position P36 of SEQ ID NO: 1, an I at position V93 of SEQ ID NO: 1, and an A at position R97 of SEQ ID NO: 1.

### Y7M_I16W_P90F_V93I

In some embodiments, a KOD polymerase variant comprises: a M at a position corresponding to Y7 of SEQ ID NO: 1, a W at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, a F at position P90 of SEQ ID NO: 1, and an I at position V93 of SEQ ID NO: 1.

### Y7M_I16W_P90F_V93I_R97A

In some embodiments, a KOD polymerase variant comprises: a M at a position corresponding to Y7 of SEQ ID NO: 1, a W at a position corresponding to I16 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, a F at position P90 of SEQ ID NO: 1, an I at position V93 of SEQ ID NO: 1, and an A at position R97 of SEQ ID NO: 1.

### Y7M_I16W_P90I

In some embodiments, a KOD polymerase variant comprises: a M at a position corresponding to Y7 of SEQ ID NO: 1, a W at a position corresponding to I16 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and an I at position P90 of SEQ ID NO: 1. In some embodiments, a KOD polymerase variant comprises: a M at position Y7 of SEQ ID NO: 1, a W at position I16 of SEQ ID NO: 1, and an I at position P90 of SEQ ID NO: 1.

### Y7M_I16W_P90I_V93I

In some embodiments, a KOD polymerase variant comprises: a M at a position corresponding to Y7 of SEQ ID NO: 1, a W at a position corresponding to I16 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, an I at position P90 of SEQ ID NO: 1, and an I at position V93 of SEQ ID NO: 1.

### Y7M_I16W_P90I_V93I_R97A

In some embodiments, a KOD polymerase variant comprises: a M at a position corresponding to Y7 of SEQ ID NO: 1, a W at a position corresponding to I16 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, an I at position P90 of SEQ ID NO: 1, an I at position V93 of SEQ ID NO: 1, and an A at position R97 of SEQ ID NO: 1.

### Y7M_I16W_P90W

In some embodiments, a KOD polymerase variant comprises: a M at a position corresponding to Y7 of SEQ ID NO: 1, a W at a position corresponding to I16 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a W at position P90 of SEQ ID NO: 1. In some embodiments, a KOD polymerase variant comprises: a M at position Y7 of SEQ ID NO: 1, a W at position I16 of SEQ ID NO: 1, and a W at position P90 of SEQ ID NO: 1.

### Y7M_I16W_P90W_V93I

In some embodiments, a KOD polymerase variant comprises: a M at a position corresponding to Y7 of SEQ ID NO: 1, a W at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, a W at position P90 of SEQ ID NO: 1, and an I at position V93 of SEQ ID NO: 1.

### Y7M_I16W_P90W_V93I_R97F

In some embodiments, a KOD polymerase variant comprises: a M at a position corresponding to Y7 of SEQ ID NO: 1, a W at a position corresponding to I16 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position I16 of SEQ ID NO: 1, a W at position P90 of SEQ ID NO: 1, an I at position V93 of SEQ ID NO: 1, and a F at position R97 of SEQ ID NO: 1.

### Y7M_I16W_P90Y

In some embodiments, a KOD polymerase variant comprises: a M at a position corresponding to Y7 of SEQ ID NO: 1, a W at a position corresponding to I16 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a Y at position P90 of SEQ ID NO: 1. In some embodiments, a KOD polymerase variant comprises: a M at position Y7 of SEQ ID NO: 1, a W at position I16 of SEQ ID NO: 1, and a Y at position P90 of SEQ ID NO: 1.

### Y7M_I16W_P90Y_R97A

In some embodiments, a KOD polymerase variant comprises: a M at a position corresponding to Y7 of SEQ ID NO: 1, a W at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, a Y at position P90 of SEQ ID NO: 1, and an A at position R97 of SEQ ID NO: 1.

### Y7M_I16W_P90Y_V93I

In some embodiments, a KOD polymerase variant comprises: a M at a position corresponding to Y7 of SEQ ID NO: 1, a W at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, a Y at position P90 of SEQ ID NO: 1, and an I at position V93 of SEQ ID NO: 1.

### Y7M_I16W_R97A

In some embodiments, a KOD polymerase variant comprises: a M at a position corresponding to Y7 of SEQ ID NO: 1, a W at a position corresponding to I16 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, and an A at position R97 of SEQ ID NO: 1.

### Y7M_I16W_R97F

In some embodiments, a KOD polymerase variant comprises: a M at a position corresponding to Y7 of SEQ ID NO: 1, a W at a position corresponding to I16 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a F at position R97 of SEQ ID NO: 1. In some embodiments, a KOD polymerase variant comprises: a M at position Y7 of SEQ ID NO: 1, a W at position I16 of SEQ ID NO: 1, and a F at position R97 of SEQ ID NO: 1.

### Y7M_I16W_R97H

In some embodiments, a KOD polymerase variant comprises: a M at a position corresponding to Y7 of SEQ ID NO: 1, a W at a position corresponding to I16 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a H at position R97 of SEQ ID NO: 1. In some embodiments, a KOD polymerase variant comprises: a M at position Y7 of SEQ ID NO: 1, a W at position I16 of SEQ ID NO: 1, and a H at position R97 of SEQ ID NO: 1.

### Y7M_I16W_R97W

In some embodiments, a KOD polymerase variant comprises: a M at a position corresponding to Y7 of SEQ ID NO: 1, a W at a position corresponding to I16 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a W at position R97 of SEQ ID NO: 1. In some embodiments, a KOD polymerase variant comprises: a M at position Y7 of SEQ ID NO: 1, a W at position I16 of SEQ ID NO: 1, and a W at position R97 of SEQ ID NO: 1.

### Y7M_I16W_V93I

In some embodiments, a KOD polymerase variant comprises: a M at a position corresponding to Y7 of SEQ ID NO: 1, a W at a position corresponding to I16 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and an I at position V93 of SEQ ID NO: 1. In some embodiments, a KOD polymerase variant comprises: a M at position Y7 of SEQ ID NO: 1, a W at position I16 of SEQ ID NO: 1, and an I at position V93 of SEQ ID NO: 1.

### Y7M_I16W_V93I_R97A

In some embodiments, a KOD polymerase variant comprises: a M at a position corresponding to Y7 of SEQ ID NO: 1, a W at a position corresponding to I16 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position I16 of SEQ ID NO: 1, an I at position V93 of SEQ ID NO: 1, and an A at position R97 of SEQ ID NO: 1.

### Y7M_I16W_V93I_R97F

In some embodiments, a KOD polymerase variant comprises: a M at a position corresponding to Y7 of SEQ ID NO: 1, a W at a position corresponding to I16 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position I16 of SEQ ID NO: 1, an I at position V93 of SEQ ID NO: 1, and a F at position R97 of SEQ ID NO: 1.

### Y7M_I16W_V93I_R97H

In some embodiments, a KOD polymerase variant comprises: a M at a position corresponding to Y7 of SEQ ID NO: 1, a W at a position corresponding to I16 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position I16 of SEQ ID NO: 1, an I at position V93 of SEQ ID NO: 1, and a H at position R97 of SEQ ID NO: 1.

### Y7M_I16W_V93I_R97W

In some embodiments, a KOD polymerase variant comprises: a M at a position corresponding to Y7 of SEQ ID NO: 1, a W at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a W at position I16 of SEQ ID NO: 1, an I at position V93 of SEQ ID NO: 1, and a W at position R97 of SEQ ID NO: 1.

### Y7M_I16W_V93Q

In some embodiments, a KOD polymerase variant comprises: a M at a position corresponding to Y7 of SEQ ID NO: 1, a W at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a Q at position V93 of SEQ ID NO: 1. In some embodiments, a KOD polymerase variant comprises: a M at position Y7 of SEQ ID NO: 1, a W at position I16 of SEQ ID NO: 1, and a Q at position V93 of SEQ ID NO: 1.

### Y7M_I16W_V93Q_R97F

In some embodiments, a KOD polymerase variant comprises: a M at a position corresponding to Y7 of SEQ ID NO: 1, a W at a position corresponding to I16 of SEQ ID NO: 1, a Q at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a Q at position V93 of SEQ ID NO: 1, and a F at position R97 of SEQ ID NO: 1. In some embodiments, a KOD polymerase variant comprises: a M at position Y7 of SEQ ID NO: 1, a W at position I16 of SEQ ID NO: 1, a Q at position V93 of SEQ ID NO: 1, and a F at position R97 of SEQ ID NO: 1.

### Y7M_I16W_V93R

In some embodiments, a KOD polymerase variant comprises: a M at a position corresponding to Y7 of SEQ ID NO: 1, a W at a position corresponding to I16 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, and a R at position V93 of SEQ ID NO: 1. In some embodiments, a KOD polymerase variant comprises: a M at position Y7 of SEQ ID NO: 1, a W at position I16 of SEQ ID NO: 1, and a R at position V93 of SEQ ID NO: 1.

### Y7M_I16W_V93R_R97F

In some embodiments, a KOD polymerase variant comprises: a M at a position corresponding to Y7 of SEQ ID NO: 1, a W at a position corresponding to I16 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position I16 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a W at position I16 of SEQ ID NO: 1, a R at position V93 of SEQ ID NO: 1, and a F at position R97 of SEQ ID NO: 1.

### Y7M_P36A

In some embodiments, a KOD polymerase variant comprises: a M at a position corresponding to Y7 of SEQ ID NO: 1 and an A at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and an A at position P36 of SEQ ID NO: 1. In some embodiments, a KOD polymerase variant comprises: a M at position Y7 of SEQ ID NO: 1, and an A at position P36 of SEQ ID NO: 1.

### Y7M_P36A_P90F

In some embodiments, a KOD polymerase variant comprises: a M at a position corresponding to Y7 of SEQ ID NO: 1, an A at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a F at position P90 of SEQ ID NO: 1. In some embodiments, a KOD polymerase variant comprises: a M at position Y7 of SEQ ID NO: 1, an A at position P36 of SEQ ID NO: 1, and a F at position P90 of SEQ ID NO: 1.

### Y7M_P36A_P90F_V93I

In some embodiments, a KOD polymerase variant comprises: a M at a position corresponding to Y7 of SEQ ID NO: 1, an A at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, an A at position P36 of SEQ ID NO: 1, a F at position P90 of SEQ ID NO: 1, and an I at position V93 of SEQ ID NO: 1.

### Y7M_P36A_P90F_V93I_R97H

In some embodiments, a KOD polymerase variant comprises: a M at a position corresponding to Y7 of SEQ ID NO: 1, an A at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, an A at position P36 of SEQ ID NO: 1, a F at position P90 of SEQ ID NO: 1, an I at position V93 of SEQ ID NO: 1, and a H at position R97 of SEQ ID NO: 1.

### Y7M_P36A_P90F_V93Q

In some embodiments, a KOD polymerase variant comprises: a M at a position corresponding to Y7 of SEQ ID NO: 1, an A at a position corresponding to P36 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a F at position P90 of SEQ ID NO: 1, and a Q at position V93 of SEQ ID NO: 1. In some embodiments, a KOD polymerase variant comprises: a M at position Y7 of SEQ ID NO: 1, an A at position P36 of SEQ ID NO: 1, a F at position P90 of SEQ ID NO: 1, and a Q at position V93 of SEQ ID NO: 1.

### Y7M_P36A_P90I

In some embodiments, a KOD polymerase variant comprises: a M at a position corresponding to Y7 of SEQ ID NO: 1, an A at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and an I at position P90 of SEQ ID NO: 1. In some embodiments, a KOD polymerase variant comprises: a M at position Y7 of SEQ ID NO: 1, an A at position P36 of SEQ ID NO: 1, and an I at position P90 of SEQ ID NO: 1.

### Y7M_P36A_P90I_R97A

In some embodiments, a KOD polymerase variant comprises: a M at a position corresponding to Y7 of SEQ ID NO: 1, an A at a position corresponding to P36 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, an I at position P90 of SEQ ID NO: 1, and an A at position R97 of SEQ ID NO: 1. In some embodiments, a KOD polymerase variant comprises: a M at position Y7 of SEQ ID NO: 1, an A at position P36 of SEQ ID NO: 1, an I at position P90 of SEQ ID NO: 1, and an A at position R97 of SEQ ID NO: 1.

### Y7M_P36A_P90I_V93I

In some embodiments, a KOD polymerase variant comprises: a M at a position corresponding to Y7 of SEQ ID NO: 1, an A at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, an A at position P36 of SEQ ID NO: 1, an I at position P90 of SEQ ID NO: 1, and an I at position V93 of SEQ ID NO: 1.

### Y7M_P36A_P90I_V93I_R97A

In some embodiments, a KOD polymerase variant comprises: a M at a position corresponding to Y7 of SEQ ID NO: 1, an A at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, an A at position P36 of SEQ ID NO: 1, an I at position P90 of SEQ ID NO: 1, an I at position V93 of SEQ ID NO: 1, and an A at position R97 of SEQ ID NO: 1.

### Y7M_P36A_P90S

In some embodiments, a KOD polymerase variant comprises: a M at a position corresponding to Y7 of SEQ ID NO: 1, an A at a position corresponding to P36 of SEQ ID NO: 1, and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a S at position P90 of SEQ ID NO: 1. In some embodiments, a KOD polymerase variant comprises: a M at position Y7 of SEQ ID NO: 1, an A at position P36 of SEQ ID NO: 1, and a S at position P90 of SEQ ID NO: 1.

### Y7M_P36A_P90W

In some embodiments, a KOD polymerase variant comprises: a M at a position corresponding to Y7 of SEQ ID NO: 1, an A at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a W at position P90 of SEQ ID NO: 1. In some embodiments, a KOD polymerase variant comprises: a M at position Y7 of SEQ ID NO: 1, an A at position P36 of SEQ ID NO: 1, and a W at position P90 of SEQ ID NO: 1.

### Y7M_P36A_P90W_R97W

In some embodiments, a KOD polymerase variant comprises: a M at a position corresponding to Y7 of SEQ ID NO: 1, an A at a position corresponding to P36 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a W at position P90 of SEQ ID NO: 1, and a W at position R97 of SEQ ID NO: 1. In some embodiments, a KOD polymerase variant comprises: a M at position Y7 of SEQ ID NO: 1, an A at position P36 of SEQ ID NO: 1, a W at position P90 of SEQ ID NO: 1, and a W at position R97 of SEQ ID NO: 1.

### Y7M_P36A_P90W_V93I

In some embodiments, a KOD polymerase variant comprises: a M at a position corresponding to Y7 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, an A at position P36 of SEQ ID NO: 1, a W at position P90 of SEQ ID NO: 1, and an I at position V93 of SEQ ID NO: 1.

### Y7M_P36A_P90W_V93I_R97A

In some embodiments, a KOD polymerase variant comprises: a M at a position corresponding to Y7 of SEQ ID NO: 1, an A at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, an A at position P36 of SEQ ID NO: 1, a W at position P90 of SEQ ID NO: 1, an I at position V93 of SEQ ID NO: 1, and an A at position R97 of SEQ ID NO: 1.

### Y7M_P36A_P90Y_R97A

In some embodiments, a KOD polymerase variant comprises: a M at a position corresponding to Y7 of SEQ ID NO: 1, an A at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, an A at position P36 of SEQ ID NO: 1, a Y at position P90 of SEQ ID NO: 1, and an A at position R97 of SEQ ID NO: 1.

### Y7M_P36A_P90Y_V93I

In some embodiments, a KOD polymerase variant comprises: a M at a position corresponding to Y7 of SEQ ID NO: 1, an A at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, an A at position P36 of SEQ ID NO: 1, a Y at position P90 of SEQ ID NO: 1, and an I at position V93 of SEQ ID NO: 1.

### Y7M_P36A_P90Y_V93I_R97W

In some embodiments, a KOD polymerase variant comprises: a M at a position corresponding to Y7 of SEQ ID NO: 1, an A at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, an A at position P36 of SEQ ID NO: 1, a Y at position P90 of SEQ ID NO: 1, an I at position V93 of SEQ ID NO: 1, and a W at position R97 of SEQ ID NO: 1.

### Y7M_P36A_R97A

In some embodiments, a KOD polymerase variant comprises: a M at a position corresponding to Y7 of SEQ ID NO: 1, an A at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and an A at position R97 of SEQ ID NO: 1. In some embodiments, a KOD polymerase variant comprises: a M at position Y7 of SEQ ID NO: 1, an A at position P36 of SEQ ID NO: 1, and an A at position R97 of SEQ ID NO: 1.

### Y7M_P36A_R97F

In some embodiments, a KOD polymerase variant comprises: a M at a position corresponding to Y7 of SEQ ID NO: 1, an A at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a F at position R97 of SEQ ID NO: 1. In some embodiments, a KOD polymerase variant comprises: a M at position Y7 of SEQ ID NO: 1, an A at position P36 of SEQ ID NO: 1, and a F at position R97 of SEQ ID NO: 1.

### Y7M_P36A_R97H

In some embodiments, a KOD polymerase variant comprises: a M at a position corresponding to Y7 of SEQ ID NO: 1, an A at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a H at position R97 of SEQ ID NO: 1. In some embodiments, a KOD polymerase variant comprises: a M at position Y7 of SEQ ID NO: 1, an A at position P36 of SEQ ID NO: 1, and a H at position R97 of SEQ ID NO: 1.

### Y7M_P36A_V93I

In some embodiments, a KOD polymerase variant comprises: a M at a position corresponding to Y7 of SEQ ID NO: 1, an A at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and an I at position V93 of SEQ ID NO: 1. In some embodiments, a KOD polymerase variant comprises: a M at position Y7 of SEQ ID NO: 1, an A at position P36 of SEQ ID NO: 1, and an I at position V93 of SEQ ID NO: 1.

### Y7M_P36A_V93I_R97H

In some embodiments, a KOD polymerase variant comprises: a M at a position corresponding to Y7 of SEQ ID NO: 1, an A at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, an A at position P36 of SEQ ID NO: 1, an I at position V93 of SEQ ID NO: 1, and a H at position R97 of SEQ ID NO: 1.

### Y7M_P36A_V93K

In some embodiments, a KOD polymerase variant comprises: a M at a position corresponding to Y7 of SEQ ID NO: 1, an A at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a K at position V93 of SEQ ID NO: 1. In some embodiments, a KOD polymerase variant comprises: a M at position Y7 of SEQ ID NO: 1, an A at position P36 of SEQ ID NO: 1, and a K at position V93 of SEQ ID NO: 1.

### Y7M_P36A_V93K_R97W

In some embodiments, a KOD polymerase variant comprises: a M at a position corresponding to Y7 of SEQ ID NO: 1, an A at a position corresponding to P36 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a K at position V93 of SEQ ID NO: 1, and a W at position R97 of SEQ ID NO: 1. In some embodiments, a KOD polymerase variant comprises: a M at position Y7 of SEQ ID NO: 1, an A at position P36 of SEQ ID NO: 1, a K at position V93 of SEQ ID NO: 1, and a W at position R97 of SEQ ID NO: 1.

### Y7M_P36A_V93Q

In some embodiments, a KOD polymerase variant comprises: a M at a position corresponding to Y7 of SEQ ID NO: 1, an A at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a Q at position V93 of SEQ ID NO: 1. In some embodiments, a KOD polymerase variant comprises: a M at position Y7 of SEQ ID NO: 1, an A at position P36 of SEQ ID NO: 1, and a Q at position V93 of SEQ ID NO: 1.

### Y7M_P36A_V93R

In some embodiments, a KOD polymerase variant comprises: a M at a position corresponding to Y7 of SEQ ID NO: 1, an A at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, and a R at position V93 of SEQ ID NO: 1. In some embodiments, a KOD polymerase variant comprises: a M at position Y7 of SEQ ID NO: 1, an A at position P36 of SEQ ID NO: 1, and a R at position V93 of SEQ ID NO: 1.

### Y7M_P36A_V93R_R97W

In some embodiments, a KOD polymerase variant comprises: a M at a position corresponding to Y7 of SEQ ID NO: 1, an A at a position corresponding to P36 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an A at position P36 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, an A at position P36 of SEQ ID NO: 1, a R at position V93 of SEQ ID NO: 1, and a W at position R97 of SEQ ID NO: 1.

### Y7M_P36H

In some embodiments, a KOD polymerase variant comprises: a M at a position corresponding to Y7 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a H at position P36 of SEQ ID NO: 1. In some embodiments, a KOD polymerase variant comprises: a M at position Y7 of SEQ ID NO: 1, and a H at position P36 of SEQ ID NO: 1.

### Y7M_P36H_P90F

In some embodiments, a KOD polymerase variant comprises: a M at a position corresponding to Y7 of SEQ ID NO: 1, a H at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and a F at position P90 of SEQ ID NO: 1. In some embodiments, a KOD polymerase variant comprises: a M at position Y7 of SEQ ID NO: 1, a H at position P36 of SEQ ID NO: 1, and a F at position P90 of SEQ ID NO: 1.

### Y7M_P36H_P90F_V93I

In some embodiments, a KOD polymerase variant comprises: a M at a position corresponding to Y7 of SEQ ID NO: 1, a H at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a H at position P36 of SEQ ID NO: 1, a F at position P90 of SEQ ID NO: 1, and an I at position V93 of SEQ ID NO: 1.

### Y7M_P36H_P90I

In some embodiments, a KOD polymerase variant comprises: a M at a position corresponding to Y7 of SEQ ID NO: 1, a H at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and an I at position P90 of SEQ ID NO: 1. In some embodiments, a KOD polymerase variant comprises: a M at position Y7 of SEQ ID NO: 1, a H at position P36 of SEQ ID NO: 1, and an I at position P90 of SEQ ID NO: 1.

### Y7M_P36H_P90I_V93I

In some embodiments, a KOD polymerase variant comprises: a M at a position corresponding to Y7 of SEQ ID NO: 1, a H at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a H at position P36 of SEQ ID NO: 1, an I at position P90 of SEQ ID NO: 1, and an I at position V93 of SEQ ID NO: 1.

### Y7M_P36H_P90W

In some embodiments, a KOD polymerase variant comprises: a M at a position corresponding to Y7 of SEQ ID NO: 1, a H at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and a W at position P90 of SEQ ID NO: 1. In some embodiments, a KOD polymerase variant comprises: a M at position Y7 of SEQ ID NO: 1, a H at position P36 of SEQ ID NO: 1, and a W at position P90 of SEQ ID NO: 1.

### Y7M_P36H_P90W_V93I

In some embodiments, a KOD polymerase variant comprises: a M at a position corresponding to Y7 of SEQ ID NO: 1, a H at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a H at position P36 of SEQ ID NO: 1, a W at position P90 of SEQ ID NO: 1, and an I at position V93 of SEQ ID NO: 1.

### Y7M_P36H_P90Y

In some embodiments, a KOD polymerase variant comprises: a M at a position corresponding to Y7 of SEQ ID NO: 1, a H at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and a Y at position P90 of SEQ ID NO: 1. In some embodiments, a KOD polymerase variant comprises: a M at position Y7 of SEQ ID NO: 1, a H at position P36 of SEQ ID NO: 1, and a Y at position P90 of SEQ ID NO: 1.

### Y7M_P36H_P90Y_R97W

In some embodiments, a KOD polymerase variant comprises: a M at a position corresponding to Y7 of SEQ ID NO: 1, a H at a position corresponding to P36 of SEQ ID NO: 1, a Y at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, a Y at position P90 of SEQ ID NO: 1, and a W at position R97 of SEQ ID NO: 1. In some embodiments, a KOD polymerase variant comprises: a M at position Y7 of SEQ ID NO: 1, a H at position P36 of SEQ ID NO: 1, a Y at position P90 of SEQ ID NO: 1, and a W at position R97 of SEQ ID NO: 1.

### Y7M_P36H_P90Y_V93I

In some embodiments, a KOD polymerase variant comprises: a M at a position corresponding to Y7 of SEQ ID NO: 1, a H at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a H at position P36 of SEQ ID NO: 1, a Y at position P90 of SEQ ID NO: 1, and an I at position V93 of SEQ ID NO: 1.

### Y7M_P36H_R97F

In some embodiments, a KOD polymerase variant comprises: a M at a position corresponding to Y7 of SEQ ID NO: 1, a H at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and a F at position R97 of SEQ ID NO: 1. In some embodiments, a KOD polymerase variant comprises: a M at position Y7 of SEQ ID NO: 1, a H at position P36 of SEQ ID NO: 1, and a F at position R97 of SEQ ID NO: 1.

### Y7M_P36H_V93I

In some embodiments, a KOD polymerase variant comprises: a M at a position corresponding to Y7 of SEQ ID NO: 1, a H at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, and an I at position V93 of SEQ ID NO: 1. In some embodiments, a KOD polymerase variant comprises: a M at position Y7 of SEQ ID NO: 1, a H at position P36 of SEQ ID NO: 1, and an I at position V93 of SEQ ID NO: 1.

### Y7M_P36H_V93I_R97H

In some embodiments, a KOD polymerase variant comprises: a M at a position corresponding to Y7 of SEQ ID NO: 1, a H at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a H at position P36 of SEQ ID NO: 1, an I at position V93 of SEQ ID NO: 1, and a H at position R97 of SEQ ID NO: 1.

### Y7M_P36H_V93I_R97W

In some embodiments, a KOD polymerase variant comprises: a M at a position corresponding to Y7 of SEQ ID NO: 1, a H at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a H at position P36 of SEQ ID NO: 1, an I at position V93 of SEQ ID NO: 1, and a W at position R97 of SEQ ID NO: 1.

### Y7M_P36H_V93R_R97A

In some embodiments, a KOD polymerase variant comprises: a M at a position corresponding to Y7 of SEQ ID NO: 1, a H at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a H at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a H at position P36 of SEQ ID NO: 1, a R at position V93 of SEQ ID NO: 1, and an A at position R97 of SEQ ID NO: 1.

### Y7M_P36R

In some embodiments, a KOD polymerase variant comprises: a M at a position corresponding to Y7 of SEQ ID NO: 1 and a R at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a R at position P36 of SEQ ID NO: 1. In some embodiments, a KOD polymerase variant comprises: a M at position Y7 of SEQ ID NO: 1, and a R at position P36 of SEQ ID NO: 1.

### Y7M_P36R_R97A

In some embodiments, a KOD polymerase variant comprises: a M at a position corresponding to Y7 of SEQ ID NO: 1, a R at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a R at position P36 of SEQ ID NO: 1, and an A at position R97 of SEQ ID NO: 1. In some embodiments, a KOD polymerase variant comprises: a M at position Y7 of SEQ ID NO: 1, a R at position P36 of SEQ ID NO: 1, and an A at position R97 of SEQ ID NO: 1.

### Y7M_P36R_V93I_R97A

In some embodiments, a KOD polymerase variant comprises: a M at a position corresponding to Y7 of SEQ ID NO: 1, a R at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a R at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a R at position P36 of SEQ ID NO: 1, an I at position V93 of SEQ ID NO: 1, and an A at position R97 of SEQ ID NO: 1.

### Y7M_P36S

In some embodiments, a KOD polymerase variant comprises: a M at a position corresponding to Y7 of SEQ ID NO: 1 and a S at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a S at position P36 of SEQ ID NO: 1. In some embodiments, a KOD polymerase variant comprises: a M at position Y7 of SEQ ID NO: 1, and a S at position P36 of SEQ ID NO: 1.

### Y7M_P36S_P90F

In some embodiments, a KOD polymerase variant comprises: a M at a position corresponding to Y7 of SEQ ID NO: 1, a S at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a F at position P90 of SEQ ID NO: 1. In some embodiments, a KOD polymerase variant comprises: a M at position Y7 of SEQ ID NO: 1, a S at position P36 of SEQ ID NO: 1, and a F at position P90 of SEQ ID NO: 1.

### Y7M_P36S_P90F_V93I

In some embodiments, a KOD polymerase variant comprises: a M at a position corresponding to Y7 of SEQ ID NO: 1, a S at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a S at position P36 of SEQ ID NO: 1, a F at position P90 of SEQ ID NO: 1, and an I at position V93 of SEQ ID NO: 1.

### Y7M_P36S_P90I_V93I

In some embodiments, a KOD polymerase variant comprises: a M at a position corresponding to Y7 of SEQ ID NO: 1, a S at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a S at position P36 of SEQ ID NO: 1, an I at position P90 of SEQ ID NO: 1, and an I at position V93 of SEQ ID NO: 1.

### Y7M_P36S_P90I_V93I_R97H

In some embodiments, a KOD polymerase variant comprises: a M at a position corresponding to Y7 of SEQ ID NO: 1, a S at a position corresponding to P36 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a S at position P36 of SEQ ID NO: 1, an I at position P90 of SEQ ID NO: 1, an I at position V93 of SEQ ID NO: 1, and a H at position R97 of SEQ ID NO: 1.

### Y7M_P36S_P90W

In some embodiments, a KOD polymerase variant comprises: a M at a position corresponding to Y7 of SEQ ID NO: 1, a S at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a W at position P90 of SEQ ID NO: 1. In some embodiments, a KOD polymerase variant comprises: a M at position Y7 of SEQ ID NO: 1, a S at position P36 of SEQ ID NO: 1, and a W at position P90 of SEQ ID NO: 1.

### Y7M_P36S_P90W_V93I

In some embodiments, a KOD polymerase variant comprises: a M at a position corresponding to Y7 of SEQ ID NO: 1, a S at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a S at position P36 of SEQ ID NO: 1, a W at position P90 of SEQ ID NO: 1, and an I at position V93 of SEQ ID NO: 1.

### Y7M_P36S_P90Y_R97A

In some embodiments, a KOD polymerase variant comprises: a M at a position corresponding to Y7 of SEQ ID NO: 1, a S at a position corresponding to P36 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a Y at position P90 of SEQ ID NO: 1, and an A at position R97 of SEQ ID NO: 1. In some embodiments, a KOD polymerase variant comprises: a M at position Y7 of SEQ ID NO: 1, a S at position P36 of SEQ ID NO: 1, a Y at position P90 of SEQ ID NO: 1, and an A at position R97 of SEQ ID NO: 1.

### Y7M_P36S_P90Y_V93I

In some embodiments, a KOD polymerase variant comprises: a M at a position corresponding to Y7 of SEQ ID NO: 1, a S at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a S at position P36 of SEQ ID NO: 1, a Y at position P90 of SEQ ID NO: 1, and an I at position V93 of SEQ ID NO: 1.

### Y7M_P36S_R97A

In some embodiments, a KOD polymerase variant comprises: a M at a position corresponding to Y7 of SEQ ID NO: 1, a S at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and an A at position R97 of SEQ ID NO: 1. In some embodiments, a KOD polymerase variant comprises: a M at position Y7 of SEQ ID NO: 1, a S at position P36 of SEQ ID NO: 1, and an A at position R97 of SEQ ID NO: 1.

### Y7M_P36S_R97F

In some embodiments, a KOD polymerase variant comprises: a M at a position corresponding to Y7 of SEQ ID NO: 1, a S at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a F at position R97 of SEQ ID NO: 1. In some embodiments, a KOD polymerase variant comprises: a M at position Y7 of SEQ ID NO: 1, a S at position P36 of SEQ ID NO: 1, and a F at position R97 of SEQ ID NO: 1.

### Y7M_P36S_R97H

In some embodiments, a KOD polymerase variant comprises: a M at a position corresponding to Y7 of SEQ ID NO: 1, a S at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a H at position R97 of SEQ ID NO: 1. In some embodiments, a KOD polymerase variant comprises: a M at position Y7 of SEQ ID NO: 1, a S at position P36 of SEQ ID NO: 1, and a H at position R97 of SEQ ID NO: 1.

### Y7M_P36S_R97W

In some embodiments, a KOD polymerase variant comprises: a M at a position corresponding to Y7 of SEQ ID NO: 1, a S at a position corresponding to P36 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a W at position R97 of SEQ ID NO: 1. In some embodiments, a KOD polymerase variant comprises: a M at position Y7 of SEQ ID NO: 1, a S at position P36 of SEQ ID NO: 1, and a W at position R97 of SEQ ID NO: 1.

### Y7M_P36S_V93I

In some embodiments, a KOD polymerase variant comprises: a M at a position corresponding to Y7 of SEQ ID NO: 1, a S at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and an I at position V93 of SEQ ID NO: 1. In some embodiments, a KOD polymerase variant comprises: a M at position Y7 of SEQ ID NO: 1, a S at position P36 of SEQ ID NO: 1, and an I at position V93 of SEQ ID NO: 1.

### Y7M_P36S_V93I_R97A

In some embodiments, a KOD polymerase variant comprises: a M at a position corresponding to Y7 of SEQ ID NO: 1, a S at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a S at position P36 of SEQ ID NO: 1, an I at position V93 of SEQ ID NO: 1, and an A at position R97 of SEQ ID NO: 1.

### Y7M_P36S_V93I_R97F

In some embodiments, a KOD polymerase variant comprises: a M at a position corresponding to Y7 of SEQ ID NO: 1, a S at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a S at position P36 of SEQ ID NO: 1, an I at position V93 of SEQ ID NO: 1, and a F at position R97 of SEQ ID NO: 1.

### Y7M_P36S_V93I_R97H

In some embodiments, a KOD polymerase variant comprises: a M at a position corresponding to Y7 of SEQ ID NO: 1, a S at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a S at position P36 of SEQ ID NO: 1, an I at position V93 of SEQ ID NO: 1, and a H at position R97 of SEQ ID NO: 1.

### Y7M_P36S_V93I_R97W

In some embodiments, a KOD polymerase variant comprises: a M at a position corresponding to Y7 of SEQ ID NO: 1, a S at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a S at position P36 of SEQ ID NO: 1, an I at position V93 of SEQ ID NO: 1, and a W at position R97 of SEQ ID NO: 1.

### Y7M_P36S_V93K

In some embodiments, a KOD polymerase variant comprises: a M at a position corresponding to Y7 of SEQ ID NO: 1, a S at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a K at position V93 of SEQ ID NO: 1. In some embodiments, a KOD polymerase variant comprises: a M at position Y7 of SEQ ID NO: 1, a S at position P36 of SEQ ID NO: 1, and a K at position V93 of SEQ ID NO: 1.

### Y7M_P36S_V93Q

In some embodiments, a KOD polymerase variant comprises: a M at a position corresponding to Y7 of SEQ ID NO: 1, a S at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, and a Q at position V93 of SEQ ID NO: 1. In some embodiments, a KOD polymerase variant comprises: a M at position Y7 of SEQ ID NO: 1, a S at position P36 of SEQ ID NO: 1, and a Q at position V93 of SEQ ID NO: 1.

### Y7M_P36S_V93Q_R97A

In some embodiments, a KOD polymerase variant comprises: a M at a position corresponding to Y7 of SEQ ID NO: 1, a S at a position corresponding to P36 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a S at position P36 of SEQ ID NO: 1, a Q at position V93 of SEQ ID NO: 1, and an A at position R97 of SEQ ID NO: 1.

### Y7M_P36S_V93R_R97A

In some embodiments, a KOD polymerase variant comprises: a M at a position corresponding to Y7 of SEQ ID NO: 1, a S at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a S at position P36 of SEQ ID NO: 1, a R at position V93 of SEQ ID NO: 1, and an A at position R97 of SEQ ID NO: 1.

### Y7M_P36S_V93R_R97F

In some embodiments, a KOD polymerase variant comprises: a M at a position corresponding to Y7 of SEQ ID NO: 1, a S at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P36 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a S at position P36 of SEQ ID NO: 1, a R at position V93 of SEQ ID NO: 1, and a F at position R97 of SEQ ID NO: 1.

### Y7M_P36W

In some embodiments, a KOD polymerase variant comprises: a M at a position corresponding to Y7 of SEQ ID NO: 1 and a W at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a W at position P36 of SEQ ID NO: 1. In some embodiments, a KOD polymerase variant comprises: a M at position Y7 of SEQ ID NO: 1, and a W at position P36 of SEQ ID NO: 1.

### Y7M_P36W_P90F

In some embodiments, a KOD polymerase variant comprises: a M at a position corresponding to Y7 of SEQ ID NO: 1, a W at a position corresponding to P36 of SEQ ID NO: 1, and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a F at position P90 of SEQ ID NO: 1. In some embodiments, a KOD polymerase variant comprises: a M at position Y7 of SEQ ID NO: 1, a W at position P36 of SEQ ID NO: 1, and a F at position P90 of SEQ ID NO: 1.

### Y7M_P36W_P90F_R97A

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, and an A at position R97 of SEQ ID NO: 1.

### Y7M_P36W_P90F_R97H

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, and a H at position R97 of SEQ ID NO: 1.

### Y7M_P36W_P90F_V93I

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, and an I at position V93 of SEQ ID NO: 1.

### Y7M_P36W_P90F_V93I_R97A

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, an I at position V93 of SEQ ID NO: 1, and an A at position R97 of SEQ ID NO: 1.

### Y7M_P36W_P90F_V93I_R97F

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, an I at position V93 of SEQ ID NO: 1, and a F at position R97 of SEQ ID NO: 1.

### Y7M_P36W_P90F_V93I_R97H

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, an I at position V93 of SEQ ID NO: 1, and a H at position R97 of SEQ ID NO: 1.

### Y7M_P36W_P90F_V93I_R97W

In some embodiments, a KOD polymerase variant comprises: a M at a position corresponding to Y7 of SEQ ID NO: 1, a W at a position corresponding to P36 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a W at position P36 of SEQ ID NO: 1, a F at position P90 of SEQ ID NO: 1, an I at position V93 of SEQ ID NO: 1, and a W at position R97 of SEQ ID NO: 1.

### Y7M_P36W_P90I

In some embodiments, a KOD polymerase variant comprises: a M at a position corresponding to Y7 of SEQ ID NO: 1, a W at a position corresponding to P36 of SEQ ID NO: 1, and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and an I at position P90 of SEQ ID NO: 1. In some embodiments, a KOD polymerase variant comprises: a M at position Y7 of SEQ ID NO: 1, a W at position P36 of SEQ ID NO: 1, and an I at position P90 of SEQ ID NO: 1.

### Y7M_P36W_P90I_V93I

In some embodiments, a KOD polymerase variant comprises: a M at a position corresponding to Y7 of SEQ ID NO: 1, a W at a position corresponding to P36 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, a W at position P36 of SEQ ID NO: 1, an I at position P90 of SEQ ID NO: 1, and an I at position V93 of SEQ ID NO: 1.

### Y7M_P36W_P90I_V93I_R97A

In some embodiments, a KOD polymerase variant comprises: a M at a position corresponding to Y7 of SEQ ID NO: 1, a W at a position corresponding to P36 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, an I at position P90 of SEQ ID NO: 1, an I at position V93 of SEQ ID NO: 1, and an A at position R97 of SEQ ID NO: 1.

### Y7M_P36W_P90I_V93R

In some embodiments, a KOD polymerase variant comprises: a M at a position corresponding to Y7 of SEQ ID NO: 1, a W at a position corresponding to P36 of SEQ ID NO: 1, an I at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position P90 of SEQ ID NO: 1, and a R at position V93 of SEQ ID NO: 1. In some embodiments, a KOD polymerase variant comprises: a M at position Y7 of SEQ ID NO: 1, a W at position P36 of SEQ ID NO: 1, an I at position P90 of SEQ ID NO: 1, and a R at position V93 of SEQ ID NO: 1.

### Y7M_P36W_P90S

In some embodiments, a KOD polymerase variant comprises: a M at a position corresponding to Y7 of SEQ ID NO: 1, a W at a position corresponding to P36 of SEQ ID NO: 1, and a S at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a S at position P90 of SEQ ID NO: 1. In some embodiments, a KOD polymerase variant comprises: a M at position Y7 of SEQ ID NO: 1, a W at position P36 of SEQ ID NO: 1, and a S at position P90 of SEQ ID NO: 1.

### Y7M_P36W_P90S_V93I_R97A

In some embodiments, a KOD polymerase variant comprises: a M at a position corresponding to Y7 of SEQ ID NO: 1, a W at a position corresponding to P36 of SEQ ID NO: 1, a S at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a S at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a S at position P90 of SEQ ID NO: 1, an I at position V93 of SEQ ID NO: 1, and an A at position R97 of SEQ ID NO: 1.

### Y7M_P36W_P90W

In some embodiments, a KOD polymerase variant comprises: a M at a position corresponding to Y7 of SEQ ID NO: 1, a W at a position corresponding to P36 of SEQ ID NO: 1, and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a W at position P90 of SEQ ID NO: 1. In some embodiments, a KOD polymerase variant comprises: a M at position Y7 of SEQ ID NO: 1, a W at position P36 of SEQ ID NO: 1, and a W at position P90 of SEQ ID NO: 1.

### Y7M_P36W_P90W_R97A

In some embodiments, a KOD polymerase variant comprises: a M at a position corresponding to Y7 of SEQ ID NO: 1, a W at a position corresponding to P36 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a W at position P90 of SEQ ID NO: 1, and an A at position R97 of SEQ ID NO: 1.

### Y7M_P36W_P90W_V93I

In some embodiments, a KOD polymerase variant comprises: a M at a position corresponding to Y7 of SEQ ID NO: 1, a W at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a W at position P36 of SEQ ID NO: 1, a W at position P90 of SEQ ID NO: 1, and an I at position V93 of SEQ ID NO: 1.

### Y7M_P36W_P90W_V93I_R97A

In some embodiments, a KOD polymerase variant comprises: a M at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a W at position P90 of SEQ ID NO: 1, an I at position V93 of SEQ ID NO: 1, and an A at position R97 of SEQ ID NO: 1.

### Y7M_P36W_P90W_V93I_R97F

In some embodiments, a KOD polymerase variant comprises: a M at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position P36 of SEQ ID NO: 1, a W at position P90 of SEQ ID NO: 1, an I at position V93 of SEQ ID NO: 1, and a F at position R97 of SEQ ID NO: 1.

### Y7M_P36W_P90W_V93I_R97H

In some embodiments, a KOD polymerase variant comprises: a M at a position corresponding to Y7 of SEQ ID NO: 1, a W at a position corresponding to P36 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, a W at position P90 of SEQ ID NO: 1, an I at position V93 of SEQ ID NO: 1, and a H at position R97 of SEQ ID NO: 1.

### Y7M_P36W_P90Y

In some embodiments, a KOD polymerase variant comprises: a M at a position corresponding to Y7 of SEQ ID NO: 1, a W at a position corresponding to P36 of SEQ ID NO: 1, and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a Y at position P90 of SEQ ID NO: 1. In some embodiments, a KOD polymerase variant comprises: a M at position Y7 of SEQ ID NO: 1, a W at position P36 of SEQ ID NO: 1, and a Y at position P90 of SEQ ID NO: 1.

### Y7M_P36W_P90Y_V93I

In some embodiments, a KOD polymerase variant comprises: a M at a position corresponding to Y7 of SEQ ID NO: 1, a W at a position corresponding to P36 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a W at position P36 of SEQ ID NO: 1, a Y at position P90 of SEQ ID NO: 1, and an I at position V93 of SEQ ID NO: 1.

### Y7M_P36W_P90Y_V93I_R97A

In some embodiments, a KOD polymerase variant comprises: a M at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a Y at position P90 of SEQ ID NO: 1, an I at position V93 of SEQ ID NO: 1, and an A at position R97 of SEQ ID NO: 1.

### Y7M_P36W_P90Y_V93I_R97H

In some embodiments, a KOD polymerase variant comprises: a M at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, a Y at position P90 of SEQ ID NO: 1, an I at position V93 of SEQ ID NO: 1, and a H at position R97 of SEQ ID NO: 1.

### Y7M_P36W_P90Y_V93I_R97W

In some embodiments, a KOD polymerase variant comprises: a M at a position corresponding to Y7 of SEQ ID NO: 1, a W at a position corresponding to P36 of SEQ ID NO: 1, a Y at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a W at position P36 of SEQ ID NO: 1, a Y at position P90 of SEQ ID NO: 1, an I at position V93 of SEQ ID NO: 1, and a W at position R97 of SEQ ID NO: 1.

### Y7M_P36W_P90Y_V93R

In some embodiments, a KOD polymerase variant comprises: a M at a position corresponding to Y7 of SEQ ID NO: 1, a W at a position corresponding to P36 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a Y at position P90 of SEQ ID NO: 1, and a R at position V93 of SEQ ID NO: 1. In some embodiments, a KOD polymerase variant comprises: a M at position Y7 of SEQ ID NO: 1, a W at position P36 of SEQ ID NO: 1, a Y at position P90 of SEQ ID NO: 1, and a R at position V93 of SEQ ID NO: 1.

### Y7M_P36W_R97A

In some embodiments, a KOD polymerase variant comprises: a M at a position corresponding to Y7 of SEQ ID NO: 1, a W at a position corresponding to P36 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, and an A at position R97 of SEQ ID NO: 1.

### Y7M_P36W_R97F

In some embodiments, a KOD polymerase variant comprises: a M at a position corresponding to Y7 of SEQ ID NO: 1, a W at a position corresponding to P36 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a F at position R97 of SEQ ID NO: 1. In some embodiments, a KOD polymerase variant comprises: a M at position Y7 of SEQ ID NO: 1, a W at position P36 of SEQ ID NO: 1, and a F at position R97 of SEQ ID NO: 1.

### Y7M_P36W_R97H

In some embodiments, a KOD polymerase variant comprises: a M at a position corresponding to Y7 of SEQ ID NO: 1, a W at a position corresponding to P36 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, and a H at position R97 of SEQ ID NO: 1.

### Y7M_P36W_V93I

In some embodiments, a KOD polymerase variant comprises: a M at a position corresponding to Y7 of SEQ ID NO: 1, a W at a position corresponding to P36 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and an I at position V93 of SEQ ID NO: 1. In some embodiments, a KOD polymerase variant comprises: a M at position Y7 of SEQ ID NO: 1, a W at position P36 of SEQ ID NO: 1, and an I at position V93 of SEQ ID NO: 1.

### Y7M_P36W_V93I_R97A

In some embodiments, a KOD polymerase variant comprises: a M at a position corresponding to Y7 of SEQ ID NO: 1, a W at a position corresponding to P36 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, an I at position V93 of SEQ ID NO: 1, and an A at position R97 of SEQ ID NO: 1.

### Y7M_P36W_V93I_R97F

In some embodiments, a KOD polymerase variant comprises: a M at a position corresponding to Y7 of SEQ ID NO: 1, a W at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position P36 of SEQ ID NO: 1, an I at position V93 of SEQ ID NO: 1, and a F at position R97 of SEQ ID NO: 1.

### Y7M_P36W_V93I_R97H

In some embodiments, a KOD polymerase variant comprises: a M at a position corresponding to Y7 of SEQ ID NO: 1, a W at a position corresponding to P36 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position P36 of SEQ ID NO: 1, an I at position V93 of SEQ ID NO: 1, and a H at position R97 of SEQ ID NO: 1.

### Y7M_P36W_V93I_R97W

In some embodiments, a KOD polymerase variant comprises: a M at a position corresponding to Y7 of SEQ ID NO: 1, a W at a position corresponding to P36 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a W at position P36 of SEQ ID NO: 1, an I at position V93 of SEQ ID NO: 1, and a W at position R97 of SEQ ID NO: 1.

### Y7M_P36W_V93K

In some embodiments, a KOD polymerase variant comprises: a M at a position corresponding to Y7 of SEQ ID NO: 1, a W at a position corresponding to P36 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a K at position V93 of SEQ ID NO: 1. In some embodiments, a KOD polymerase variant comprises: a M at position Y7 of SEQ ID NO: 1, a W at position P36 of SEQ ID NO: 1, and a K at position V93 of SEQ ID NO: 1.

### Y7M_P36W_V93Q

In some embodiments, a KOD polymerase variant comprises: a M at a position corresponding to Y7 of SEQ ID NO: 1, a W at a position corresponding to P36 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a Q at position V93 of SEQ ID NO: 1. In some embodiments, a KOD polymerase variant comprises: a M at position Y7 of SEQ ID NO: 1, a W at position P36 of SEQ ID NO: 1, and a Q at position V93 of SEQ ID NO: 1.

### Y7M_P36W_V93R

In some embodiments, a KOD polymerase variant comprises: a M at a position corresponding to Y7 of SEQ ID NO: 1, a W at a position corresponding to P36 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, and a R at position V93 of SEQ ID NO: 1. In some embodiments, a KOD polymerase variant comprises: a M at position Y7 of SEQ ID NO: 1, a W at position P36 of SEQ ID NO: 1, and a R at position V93 of SEQ ID NO: 1.

### Y7M_P36W_V93R_R97A

In some embodiments, a KOD polymerase variant comprises: a M at a position corresponding to Y7 of SEQ ID NO: 1, a W at a position corresponding to P36 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a W at position P36 of SEQ ID NO: 1, a R at position V93 of SEQ ID NO: 1, and an A at position R97 of SEQ ID NO: 1.

### Y7M_P36W_V93R_R97F

In some embodiments, a KOD polymerase variant comprises: a M at a position corresponding to Y7 of SEQ ID NO: 1, a W at a position corresponding to P36 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P36 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a W at position P36 of SEQ ID NO: 1, a R at position V93 of SEQ ID NO: 1, and a F at position R97 of SEQ ID NO: 1.

### Y7M_P90F

In some embodiments, a KOD polymerase variant comprises: a M at a position corresponding to Y7 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a F at position P90 of SEQ ID NO: 1. In some embodiments, a KOD polymerase variant comprises: a M at position Y7 of SEQ ID NO: 1, and a F at position P90 of SEQ ID NO: 1.

### Y7M_P90F_R97A

In some embodiments, a KOD polymerase variant comprises: a M at a position corresponding to Y7 of SEQ ID NO: 1, a F at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and an A at position R97 of SEQ ID NO: 1. In some embodiments, a KOD polymerase variant comprises: a M at position Y7 of SEQ ID NO: 1, a F at position P90 of SEQ ID NO: 1, and an A at position R97 of SEQ ID NO: 1.

### Y7M_P90F_R97F

In some embodiments, a KOD polymerase variant comprises: a M at a position corresponding to Y7 of SEQ ID NO: 1, a F at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and a F at position R97 of SEQ ID NO: 1. In some embodiments, a KOD polymerase variant comprises: a M at position Y7 of SEQ ID NO: 1, a F at position P90 of SEQ ID NO: 1, and a F at position R97 of SEQ ID NO: 1.

### Y7M_P90F_R97H

In some embodiments, a KOD polymerase variant comprises: a M at a position corresponding to Y7 of SEQ ID NO: 1, a F at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and a H at position R97 of SEQ ID NO: 1. In some embodiments, a KOD polymerase variant comprises: a M at position Y7 of SEQ ID NO: 1, a F at position P90 of SEQ ID NO: 1, and a H at position R97 of SEQ ID NO: 1.

### Y7M_P90F_V93I

In some embodiments, a KOD polymerase variant comprises: a M at a position corresponding to Y7 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and an I at position V93 of SEQ ID NO: 1. In some embodiments, a KOD polymerase variant comprises: a M at position Y7 of SEQ ID NO: 1, a F at position P90 of SEQ ID NO: 1, and an I at position V93 of SEQ ID NO: 1.

### Y7M_P90F_V93I_R97H

In some embodiments, a KOD polymerase variant comprises: a M at a position corresponding to Y7 of SEQ ID NO: 1, a F at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a F at position P90 of SEQ ID NO: 1, an I at position V93 of SEQ ID NO: 1, and a H at position R97 of SEQ ID NO: 1.

### Y7M_P90F_V93I_R97W

In some embodiments, a KOD polymerase variant comprises: a M at a position corresponding to Y7 of SEQ ID NO: 1, a F at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a F at position P90 of SEQ ID NO: 1, an I at position V93 of SEQ ID NO: 1, and a W at position R97 of SEQ ID NO: 1.

### Y7M_P90F_V93Q

In some embodiments, a KOD polymerase variant comprises: a M at a position corresponding to Y7 of SEQ ID NO: 1, a F at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and a Q at position V93 of SEQ ID NO: 1. In some embodiments, a KOD polymerase variant comprises: a M at position Y7 of SEQ ID NO: 1, a F at position P90 of SEQ ID NO: 1, and a Q at position V93 of SEQ ID NO: 1.

### Y7M_P90F_V93R

In some embodiments, a KOD polymerase variant comprises: a M at a position corresponding to Y7 of SEQ ID NO: 1, a F at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, and a R at position V93 of SEQ ID NO: 1. In some embodiments, a KOD polymerase variant comprises: a M at position Y7 of SEQ ID NO: 1, a F at position P90 of SEQ ID NO: 1, and a R at position V93 of SEQ ID NO: 1.

### Y7M_P90F_V93R_R97A

In some embodiments, a KOD polymerase variant comprises: a M at a position corresponding to Y7 of SEQ ID NO: 1, a F at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a F at position P90 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a F at position P90 of SEQ ID NO: 1, a R at position V93 of SEQ ID NO: 1, and an A at position R97 of SEQ ID NO: 1.

### Y7M_P90I

In some embodiments, a KOD polymerase variant comprises: a M at a position corresponding to Y7 of SEQ ID NO: 1 and an I at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and an I at position P90 of SEQ ID NO: 1. In some embodiments, a KOD polymerase variant comprises: a M at position Y7 of SEQ ID NO: 1, and an I at position P90 of SEQ ID NO: 1.

### Y7M_P90I_R97A

In some embodiments, a KOD polymerase variant comprises: a M at a position corresponding to Y7 of SEQ ID NO: 1, an I at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, and an A at position R97 of SEQ ID NO: 1. In some embodiments, a KOD polymerase variant comprises: a M at position Y7 of SEQ ID NO: 1, an I at position P90 of SEQ ID NO: 1, and an A at position R97 of SEQ ID NO: 1.

### Y7M_P90I_R97F

In some embodiments, a KOD polymerase variant comprises: a M at a position corresponding to Y7 of SEQ ID NO: 1, an I at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, and a F at position R97 of SEQ ID NO: 1. In some embodiments, a KOD polymerase variant comprises: a M at position Y7 of SEQ ID NO: 1, an I at position P90 of SEQ ID NO: 1, and a F at position R97 of SEQ ID NO: 1.

### Y7M_P90I_V93I

In some embodiments, a KOD polymerase variant comprises: a M at a position corresponding to Y7 of SEQ ID NO: 1, an I at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, and an I at position V93 of SEQ ID NO: 1. In some embodiments, a KOD polymerase variant comprises: a M at position Y7 of SEQ ID NO: 1, an I at position P90 of SEQ ID NO: 1, and an I at position V93 of SEQ ID NO: 1.

### Y7M_P90I_V93I_R97A

In some embodiments, a KOD polymerase variant comprises: a M at a position corresponding to Y7 of SEQ ID NO: 1, an I at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, an I at position P90 of SEQ ID NO: 1, an I at position V93 of SEQ ID NO: 1, and an A at position R97 of SEQ ID NO: 1.

### Y7M_P90I_V93I_R97H

In some embodiments, a KOD polymerase variant comprises: a M at a position corresponding to Y7 of SEQ ID NO: 1, an I at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, an I at position P90 of SEQ ID NO: 1, an I at position V93 of SEQ ID NO: 1, and a H at position R97 of SEQ ID NO: 1.

### Y7M_P90I_V93I_R97W

In some embodiments, a KOD polymerase variant comprises: a M at a position corresponding to Y7 of SEQ ID NO: 1, an I at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, an I at position P90 of SEQ ID NO: 1, an I at position V93 of SEQ ID NO: 1, and a W at position R97 of SEQ ID NO: 1.

### Y7M_P90I_V93K

In some embodiments, a KOD polymerase variant comprises: a M at a position corresponding to Y7 of SEQ ID NO: 1, an I at a position corresponding to P90 of SEQ ID NO: 1, and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, and a K at position V93 of SEQ ID NO: 1. In some embodiments, a KOD polymerase variant comprises: a M at position Y7 of SEQ ID NO: 1, an I at position P90 of SEQ ID NO: 1, and a K at position V93 of SEQ ID NO: 1.

### Y7M_P90I_V93Q

In some embodiments, a KOD polymerase variant comprises: a M at a position corresponding to Y7 of SEQ ID NO: 1, an I at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, and a Q at position V93 of SEQ ID NO: 1. In some embodiments, a KOD polymerase variant comprises: a M at position Y7 of SEQ ID NO: 1, an I at position P90 of SEQ ID NO: 1, and a Q at position V93 of SEQ ID NO: 1.

### Y7M_P90I_V93R_R97A

In some embodiments, a KOD polymerase variant comprises: a M at a position corresponding to Y7 of SEQ ID NO: 1, an I at a position corresponding to P90 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, an I at position P90 of SEQ ID NO: 1, a R at position V93 of SEQ ID NO: 1, and an A at position R97 of SEQ ID NO: 1.

### Y7M_P90I_V93R_R97W

In some embodiments, a KOD polymerase variant comprises: a M at a position corresponding to Y7 of SEQ ID NO: 1, an I at a position corresponding to P90 of SEQ ID NO: 1, a R at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position P90 of SEQ ID NO: 1, a R at position V93 of SEQ ID NO: 1, and a W at position R97 of SEQ ID NO: 1. In some embodiments, a KOD polymerase variant comprises: a M at position Y7 of SEQ ID NO: 1, an I at position P90 of SEQ ID NO: 1, a R at position V93 of SEQ ID NO: 1, and a W at position R97 of SEQ ID NO: 1.

### Y7M_P90S_R97H

In some embodiments, a KOD polymerase variant comprises: a M at a position corresponding to Y7 of SEQ ID NO: 1, a S at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, and a H at position R97 of SEQ ID NO: 1. In some embodiments, a KOD polymerase variant comprises: a M at position Y7 of SEQ ID NO: 1, a S at position P90 of SEQ ID NO: 1, and a H at position R97 of SEQ ID NO: 1.

### Y7M_P90S_V93I

In some embodiments, a KOD polymerase variant comprises: a M at a position corresponding to Y7 of SEQ ID NO: 1, a S at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, and an I at position V93 of SEQ ID NO: 1. In some embodiments, a KOD polymerase variant comprises: a M at position Y7 of SEQ ID NO: 1, a S at position P90 of SEQ ID NO: 1, and an I at position V93 of SEQ ID NO: 1.

### Y7M_P90S_V93I_R97F

In some embodiments, a KOD polymerase variant comprises: a M at a position corresponding to Y7 of SEQ ID NO: 1, a S at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a S at position P90 of SEQ ID NO: 1, an I at position V93 of SEQ ID NO: 1, and a F at position R97 of SEQ ID NO: 1.

### Y7M_P90S_V93I_R97H

In some embodiments, a KOD polymerase variant comprises: a M at a position corresponding to Y7 of SEQ ID NO: 1, a S at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a S at position P90 of SEQ ID NO: 1, an I at position V93 of SEQ ID NO: 1, and a H at position R97 of SEQ ID NO: 1.

### Y7M_P90S_V93I_R97W

In some embodiments, a KOD polymerase variant comprises: a M at a position corresponding to Y7 of SEQ ID NO: 1, a S at a position corresponding to P90 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, a S at position P90 of SEQ ID NO: 1, an I at position V93 of SEQ ID NO: 1, and a W at position R97 of SEQ ID NO: 1.

### Y7M_P90S_V93Q

In some embodiments, a KOD polymerase variant comprises: a M at a position corresponding to Y7 of SEQ ID NO: 1, a S at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, and a Q at position V93 of SEQ ID NO: 1. In some embodiments, a KOD polymerase variant comprises: a M at position Y7 of SEQ ID NO: 1, a S at position P90 of SEQ ID NO: 1, and a Q at position V93 of SEQ ID NO: 1.

### Y7M_P90S_V93Q_R97A

In some embodiments, a KOD polymerase variant comprises: a M at a position corresponding to Y7 of SEQ ID NO: 1, a S at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a S at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a S at position P90 of SEQ ID NO: 1, a Q at position V93 of SEQ ID NO: 1, and an A at position R97 of SEQ ID NO: 1.

### Y7M_P90W

In some embodiments, a KOD polymerase variant comprises: a M at a position corresponding to Y7 of SEQ ID NO: 1 and a W at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a W at position P90 of SEQ ID NO: 1. In some embodiments, a KOD polymerase variant comprises: a M at position Y7 of SEQ ID NO: 1, and a W at position P90 of SEQ ID NO: 1.

### Y7M_P90W_R97A

In some embodiments, a KOD polymerase variant comprises: a M at a position corresponding to Y7 of SEQ ID NO: 1, a W at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and an A at position R97 of SEQ ID NO: 1. In some embodiments, a KOD polymerase variant comprises: a M at position Y7 of SEQ ID NO: 1, a W at position P90 of SEQ ID NO: 1, and an A at position R97 of SEQ ID NO: 1.

### Y7M_P90W_R97F

In some embodiments, a KOD polymerase variant comprises: a M at a position corresponding to Y7 of SEQ ID NO: 1, a W at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and a F at position R97 of SEQ ID NO: 1. In some embodiments, a KOD polymerase variant comprises: a M at position Y7 of SEQ ID NO: 1, a W at position P90 of SEQ ID NO: 1, and a F at position R97 of SEQ ID NO: 1.

### Y7M_P90W_R97H

In some embodiments, a KOD polymerase variant comprises: a M at a position corresponding to Y7 of SEQ ID NO: 1, a W at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and a H at position R97 of SEQ ID NO: 1. In some embodiments, a KOD polymerase variant comprises: a M at position Y7 of SEQ ID NO: 1, a W at position P90 of SEQ ID NO: 1, and a H at position R97 of SEQ ID NO: 1.

### Y7M_P90W_R97W

In some embodiments, a KOD polymerase variant comprises: a M at a position corresponding to Y7 of SEQ ID NO: 1, a W at a position corresponding to P90 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and a W at position R97 of SEQ ID NO: 1. In some embodiments, a KOD polymerase variant comprises: a M at position Y7 of SEQ ID NO: 1, a W at position P90 of SEQ ID NO: 1, and a W at position R97 of SEQ ID NO: 1.

### Y7M_P90W_V93I

In some embodiments, a KOD polymerase variant comprises: a M at a position corresponding to Y7 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and an I at position V93 of SEQ ID NO: 1. In some embodiments, a KOD polymerase variant comprises: a M at position Y7 of SEQ ID NO: 1, a W at position P90 of SEQ ID NO: 1, and an I at position V93 of SEQ ID NO: 1.

### Y7M_P90W_V93I_R97A

In some embodiments, a KOD polymerase variant comprises: a M at a position corresponding to Y7 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, a W at position P90 of SEQ ID NO: 1, an I at position V93 of SEQ ID NO: 1, and an A at position R97 of SEQ ID NO: 1.

### Y7M_P90W_V93I_R97F

In some embodiments, a KOD polymerase variant comprises: a M at a position corresponding to Y7 of SEQ ID NO: 1, a W at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a W at position P90 of SEQ ID NO: 1, an I at position V93 of SEQ ID NO: 1, and a F at position R97 of SEQ ID NO: 1.

### Y7M_P90W_V93I_R97H

In some embodiments, a KOD polymerase variant comprises: a M at a position corresponding to Y7 of SEQ ID NO: 1, a W at a position corresponding to P90 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, a W at position P90 of SEQ ID NO: 1, an I at position V93 of SEQ ID NO: 1, and a H at position R97 of SEQ ID NO: 1.

### Y7M_P90W_V93Q

In some embodiments, a KOD polymerase variant comprises: a M at a position corresponding to Y7 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and a Q at position V93 of SEQ ID NO: 1. In some embodiments, a KOD polymerase variant comprises: a M at position Y7 of SEQ ID NO: 1, a W at position P90 of SEQ ID NO: 1, and a Q at position V93 of SEQ ID NO: 1.

### Y7M_P90W_V93Q_R97W

In some embodiments, a KOD polymerase variant comprises: a M at a position corresponding to Y7 of SEQ ID NO: 1, a W at a position corresponding to P90 of SEQ ID NO: 1, a Q at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, a Q at position V93 of SEQ ID NO: 1, and a W at position R97 of SEQ ID NO: 1. In some embodiments, a KOD polymerase variant comprises: a M at position Y7 of SEQ ID NO: 1, a W at position P90 of SEQ ID NO: 1, a Q at position V93 of SEQ ID NO: 1, and a W at position R97 of SEQ ID NO: 1.

### Y7M_P90W_V93R

In some embodiments, a KOD polymerase variant comprises: a M at a position corresponding to Y7 of SEQ ID NO: 1, a W at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, and a R at position V93 of SEQ ID NO: 1. In some embodiments, a KOD polymerase variant comprises: a M at position Y7 of SEQ ID NO: 1, a W at position P90 of SEQ ID NO: 1, and a R at position V93 of SEQ ID NO: 1.

### Y7M_P90W_V93R_R97A_R119Q

In some embodiments, a KOD polymerase variant comprises: a M at a position corresponding to Y7 of SEQ ID NO: 1, a W at a position corresponding to P90 of SEQ ID NO: 1, a R at a position corresponding to V93 of SEQ ID NO: 1, an A at a position corresponding to R97 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, a R at position V93 of SEQ ID NO: 1, an A at position R97 of SEQ ID NO: 1, and a Q at position R119 of SEQ ID NO: 1. In some embodiments, a KOD polymerase variant comprises: a M at position Y7 of SEQ ID NO: 1, a W at position P90 of SEQ ID NO: 1, a R at position V93 of SEQ ID NO: 1, an A at position R97 of SEQ ID NO: 1, and a Q at position R119 of SEQ ID NO: 1.

### Y7M_P90W_V93R_R97F

In some embodiments, a KOD polymerase variant comprises: a M at a position corresponding to Y7 of SEQ ID NO: 1, a W at a position corresponding to P90 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a W at position P90 of SEQ ID NO: 1, a R at position V93 of SEQ ID NO: 1, and a F at position R97 of SEQ ID NO: 1.

### Y7M_P90W_V93R_R97H

In some embodiments, a KOD polymerase variant comprises: a M at a position corresponding to Y7 of SEQ ID NO: 1, a W at a position corresponding to P90 of SEQ ID NO: 1, a R at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a W at position P90 of SEQ ID NO: 1, a R at position V93 of SEQ ID NO: 1, and a H at position R97 of SEQ ID NO: 1. In some embodiments, a KOD polymerase variant comprises: a M at position Y7 of SEQ ID NO: 1, a W at position P90 of SEQ ID NO: 1, a R at position V93 of SEQ ID NO: 1, and a H at position R97 of SEQ ID NO: 1.

### Y7M_P90Y

In some embodiments, a KOD polymerase variant comprises: a M at a position corresponding to Y7 of SEQ ID NO: 1 and a Y at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a Y at position P90 of SEQ ID NO: 1. In some embodiments, a KOD polymerase variant comprises: a M at position Y7 of SEQ ID NO: 1, and a Y at position P90 of SEQ ID NO: 1.

### Y7M_P90Y_R97F

In some embodiments, a KOD polymerase variant comprises: a M at a position corresponding to Y7 of SEQ ID NO: 1, a Y at a position corresponding to P90 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, and a F at position R97 of SEQ ID NO: 1. In some embodiments, a KOD polymerase variant comprises: a M at position Y7 of SEQ ID NO: 1, a Y at position P90 of SEQ ID NO: 1, and a F at position R97 of SEQ ID NO: 1.

### Y7M_P90Y_R97H

In some embodiments, a KOD polymerase variant comprises: a M at a position corresponding to Y7 of SEQ ID NO: 1, a Y at a position corresponding to P90 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, and a H at position R97 of SEQ ID NO: 1. In some embodiments, a KOD polymerase variant comprises: a M at position Y7 of SEQ ID NO: 1, a Y at position P90 of SEQ ID NO: 1, and a H at position R97 of SEQ ID NO: 1.

### Y7M_P90Y_V93I

In some embodiments, a KOD polymerase variant comprises: a M at a position corresponding to Y7 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, and an I at position V93 of SEQ ID NO: 1. In some embodiments, a KOD polymerase variant comprises: a M at position Y7 of SEQ ID NO: 1, a Y at position P90 of SEQ ID NO: 1, and an I at position V93 of SEQ ID NO: 1.

### Y7M_P90Y_V93I_R97F

In some embodiments, a KOD polymerase variant comprises: a M at a position corresponding to Y7 of SEQ ID NO: 1, a Y at a position corresponding to P90 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, a Y at position P90 of SEQ ID NO: 1, an I at position V93 of SEQ ID NO: 1, and a F at position R97 of SEQ ID NO: 1.

### Y7M_P90Y_V93Q_R97A

In some embodiments, a KOD polymerase variant comprises: a M at a position corresponding to Y7 of SEQ ID NO: 1, a Y at a position corresponding to P90 of SEQ ID NO: 1, a Q at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, a Q at position V93 of SEQ ID NO: 1, and an A at position R97 of SEQ ID NO: 1. In some embodiments, a KOD polymerase variant comprises: a M at position Y7 of SEQ ID NO: 1, a Y at position P90 of SEQ ID NO: 1, a Q at position V93 of SEQ ID NO: 1, and an A at position R97 of SEQ ID NO: 1.

### Y7M_P90Y_V93R

In some embodiments, a KOD polymerase variant comprises: a M at a position corresponding to Y7 of SEQ ID NO: 1, a Y at a position corresponding to P90 of SEQ ID NO: 1, and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Y at position P90 of SEQ ID NO: 1, and a R at position V93 of SEQ ID NO: 1. In some embodiments, a KOD polymerase variant comprises: a M at position Y7 of SEQ ID NO: 1, a Y at position P90 of SEQ ID NO: 1, and a R at position V93 of SEQ ID NO: 1.

### Y7M_R97A

In some embodiments, a KOD polymerase variant comprises: a M at a position corresponding to Y7 of SEQ ID NO: 1 and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and an A at position R97 of SEQ ID NO: 1. In some embodiments, a KOD polymerase variant comprises: a M at position Y7 of SEQ ID NO: 1, and an A at position R97 of SEQ ID NO: 1.

### Y7M_R97F

In some embodiments, a KOD polymerase variant comprises: a M at a position corresponding to Y7 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a F at position R97 of SEQ ID NO: 1. In some embodiments, a KOD polymerase variant comprises: a M at position Y7 of SEQ ID NO: 1, and a F at position R97 of SEQ ID NO: 1.

### Y7M_R97H

In some embodiments, a KOD polymerase variant comprises: a M at a position corresponding to Y7 of SEQ ID NO: 1 and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a H at position R97 of SEQ ID NO: 1. In some embodiments, a KOD polymerase variant comprises: a M at position Y7 of SEQ ID NO: 1, and a H at position R97 of SEQ ID NO: 1.

### Y7M_R97W

In some embodiments, a KOD polymerase variant comprises: a M at a position corresponding to Y7 of SEQ ID NO: 1 and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a W at position R97 of SEQ ID NO: 1. In some embodiments, a KOD polymerase variant comprises: a M at position Y7 of SEQ ID NO: 1, and a W at position R97 of SEQ ID NO: 1.

### Y7M_V93I

In some embodiments, a KOD polymerase variant comprises: a M at a position corresponding to Y7 of SEQ ID NO: 1 and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and an I at position V93 of SEQ ID NO: 1. In some embodiments, a KOD polymerase variant comprises: a M at position Y7 of SEQ ID NO: 1, and an I at position V93 of SEQ ID NO: 1.

### Y7M_V93I_R97A

In some embodiments, a KOD polymerase variant comprises: a M at a position corresponding to Y7 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position V93 of SEQ ID NO: 1, and an A at position R97 of SEQ ID NO: 1. In some embodiments, a KOD polymerase variant comprises: a M at position Y7 of SEQ ID NO: 1, an I at position V93 of SEQ ID NO: 1, and an A at position R97 of SEQ ID NO: 1.

### Y7M_V93I_R97F

In some embodiments, a KOD polymerase variant comprises: a M at a position corresponding to Y7 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position V93 of SEQ ID NO: 1, and a F at position R97 of SEQ ID NO: 1. In some embodiments, a KOD polymerase variant comprises: a M at position Y7 of SEQ ID NO: 1, an I at position V93 of SEQ ID NO: 1, and a F at position R97 of SEQ ID NO: 1.

### Y7M_V93I_R97H

In some embodiments, a KOD polymerase variant comprises: a M at a position corresponding to Y7 of SEQ ID NO: 1, an I at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position V93 of SEQ ID NO: 1, and a H at position R97 of SEQ ID NO: 1. In some embodiments, a KOD polymerase variant comprises: a M at position Y7 of SEQ ID NO: 1, an I at position V93 of SEQ ID NO: 1, and a H at position R97 of SEQ ID NO: 1.

### Y7M_V93I_R97W

In some embodiments, a KOD polymerase variant comprises: a M at a position corresponding to Y7 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, an I at position V93 of SEQ ID NO: 1, and a W at position R97 of SEQ ID NO: 1. In some embodiments, a KOD polymerase variant comprises: a M at position Y7 of SEQ ID NO: 1, an I at position V93 of SEQ ID NO: 1, and a W at position R97 of SEQ ID NO: 1.

### Y7M_V93K

In some embodiments, a KOD polymerase variant comprises: a M at a position corresponding to Y7 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a K at position V93 of SEQ ID NO: 1. In some embodiments, a KOD polymerase variant comprises: a M at position Y7 of SEQ ID NO: 1, and a K at position V93 of SEQ ID NO: 1.

### Y7M_V93K_R97A

In some embodiments, a KOD polymerase variant comprises: a M at a position corresponding to Y7 of SEQ ID NO: 1, a K at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a K at position V93 of SEQ ID NO: 1, and an A at position R97 of SEQ ID NO: 1. In some embodiments, a KOD polymerase variant comprises: a M at position Y7 of SEQ ID NO: 1, a K at position V93 of SEQ ID NO: 1, and an A at position R97 of SEQ ID NO: 1.

### Y7M_V93K_R97F

In some embodiments, a KOD polymerase variant comprises: a M at a position corresponding to Y7 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a K at position V93 of SEQ ID NO: 1, and a F at position R97 of SEQ ID NO: 1. In some embodiments, a KOD polymerase variant comprises: a M at position Y7 of SEQ ID NO: 1, a K at position V93 of SEQ ID NO: 1, and a F at position R97 of SEQ ID NO: 1.

### Y7M_V93K_R97H

In some embodiments, a KOD polymerase variant comprises: a M at a position corresponding to Y7 of SEQ ID NO: 1, a K at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a K at position V93 of SEQ ID NO: 1, and a H at position R97 of SEQ ID NO: 1. In some embodiments, a KOD polymerase variant comprises: a M at position Y7 of SEQ ID NO: 1, a K at position V93 of SEQ ID NO: 1, and a H at position R97 of SEQ ID NO: 1.

### Y7M_V93K_R97W

In some embodiments, a KOD polymerase variant comprises: a M at a position corresponding to Y7 of SEQ ID NO: 1, a K at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a K at position V93 of SEQ ID NO: 1, and a W at position R97 of SEQ ID NO: 1. In some embodiments, a KOD polymerase variant comprises: a M at position Y7 of SEQ ID NO: 1, a K at position V93 of SEQ ID NO: 1, and a W at position R97 of SEQ ID NO: 1.

### Y7M_V93Q

In some embodiments, a KOD polymerase variant comprises: a M at a position corresponding to Y7 of SEQ ID NO: 1 and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a Q at position V93 of SEQ ID NO: 1. In some embodiments, a KOD polymerase variant comprises: a M at position Y7 of SEQ ID NO: 1, and a Q at position V93 of SEQ ID NO: 1.

### Y7M_V93Q_R97H

In some embodiments, a KOD polymerase variant comprises: a M at a position corresponding to Y7 of SEQ ID NO: 1, a Q at a position corresponding to V93 of SEQ ID NO: 1, and a H at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a Q at position V93 of SEQ ID NO: 1, and a H at position R97 of SEQ ID NO: 1. In some embodiments, a KOD polymerase variant comprises: a M at position Y7 of SEQ ID NO: 1, a Q at position V93 of SEQ ID NO: 1, and a H at position R97 of SEQ ID NO: 1.

### Y7M_V93R

In some embodiments, a KOD polymerase variant comprises: a M at a position corresponding to Y7 of SEQ ID NO: 1 and a R at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, and a R at position V93 of SEQ ID NO: 1. In some embodiments, a KOD polymerase variant comprises: a M at position Y7 of SEQ ID NO: 1, and a R at position V93 of SEQ ID NO: 1.

### Y7M_V93R_R97A

In some embodiments, a KOD polymerase variant comprises: a M at a position corresponding to Y7 of SEQ ID NO: 1, a R at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a R at position V93 of SEQ ID NO: 1, and an A at position R97 of SEQ ID NO: 1. In some embodiments, a KOD polymerase variant comprises: a M at position Y7 of SEQ ID NO: 1, a R at position V93 of SEQ ID NO: 1, and an A at position R97 of SEQ ID NO: 1.

### Y7M_V93R_R97F

In some embodiments, a KOD polymerase variant comprises: a M at a position corresponding to Y7 of SEQ ID NO: 1, a R at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position Y7 of SEQ ID NO: 1, a R at position V93 of SEQ ID NO: 1, and a F at position R97 of SEQ ID NO: 1. In some embodiments, a KOD polymerase variant comprises: a M at position Y7 of SEQ ID NO: 1, a R at position V93 of SEQ ID NO: 1, and a F at position R97 of SEQ ID NO: 1.

### Y7P_I16F_P90F_V93I

In some embodiments, a KOD polymerase variant comprises: a P at a position corresponding to Y7 of SEQ ID NO: 1, a F at a position corresponding to I16 of SEQ ID NO: 1, a F at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a F at position I16 of SEQ ID NO: 1, a F at position P90 of SEQ ID NO: 1, and an I at position V93 of SEQ ID NO: 1. In some embodiments, a KOD polymerase variant comprises: a P at position Y7 of SEQ ID NO: 1, a F at position I16 of SEQ ID NO: 1, a F at position P90 of SEQ ID NO: 1, and an I at position V93 of SEQ ID NO: 1.

### Y7P_I16F_P90Y_R97A

In some embodiments, a KOD polymerase variant comprises: a P at a position corresponding to Y7 of SEQ ID NO: 1, a M at a position corresponding to I16 of SEQ ID NO: 1, a Y at a position corresponding to P90 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a F at position I16 of SEQ ID NO: 1, a Y at position P90 of SEQ ID NO: 1, and an A at position R97 of SEQ ID NO: 1. In some embodiments, a KOD polymerase variant comprises: a P at position Y7 of SEQ ID NO: 1, a F at position I16 of SEQ ID NO: 1, a Y at position P90 of SEQ ID NO: 1, and an A at position R97 of SEQ ID NO: 1.

### Y7P_I16L_P36A_V93I_R97F

In some embodiments, a KOD polymerase variant comprises: a P at a position corresponding to Y7 of SEQ ID NO: 1, a L at a position corresponding to I16 of SEQ ID NO: 1, an A at a position corresponding to P36 of SEQ ID NO: 1, an I at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1. In some embodiments, a KOD polymerase variant comprises: a P at position Y7 of SEQ ID NO: 1, a L at position I16 of SEQ ID NO: 1, an A at position P36 of SEQ ID NO: 1, an I at position V93 of SEQ ID NO: 1, and a F at position R97 of SEQ ID NO: 1.

### Y7P_I16L_V93I_R97W

In some embodiments, a KOD polymerase variant comprises: a P at a position corresponding to Y7 of SEQ ID NO: 1, a L at a position corresponding to I16 of SEQ ID NO: 1, an I at a position corresponding to V93 of SEQ ID NO: 1, and a W at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a L at position I16 of SEQ ID NO: 1, an I at position V93 of SEQ ID NO: 1, and a W at position R97 of SEQ ID NO: 1. In some embodiments, a KOD polymerase variant comprises: a P at position Y7 of SEQ ID NO: 1, a L at position I16 of SEQ ID NO: 1, an I at position V93 of SEQ ID NO: 1, and a W at position R97 of SEQ ID NO: 1.

### Y7P_I16M_P90W_V93I

In some embodiments, a KOD polymerase variant comprises: a P at a position corresponding to Y7 of SEQ ID NO: 1, a M at a position corresponding to I16 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a M at position I16 of SEQ ID NO: 1, a W at position P90 of SEQ ID NO: 1, and an I at position V93 of SEQ ID NO: 1. In some embodiments, a KOD polymerase variant comprises: a P at position Y7 of SEQ ID NO: 1, a M at position I16 of SEQ ID NO: 1, a W at position P90 of SEQ ID NO: 1, and an I at position V93 of SEQ ID NO: 1.

### Y7P_I16M_P90W_V93I_R97A

In some embodiments, a KOD polymerase variant comprises: a P at a position corresponding to Y7 of SEQ ID NO: 1, a M at a position corresponding to I16 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a P at position Y7 of SEQ ID NO: 1, a M at position I16 of SEQ ID NO: 1, a W at position P90 of SEQ ID NO: 1, an I at position V93 of SEQ ID NO: 1, and an A at position R97 of SEQ ID NO: 1.

### Y7P_I16M_P90Y_V93I

In some embodiments, a KOD polymerase variant comprises: a P at a position corresponding to Y7 of SEQ ID NO: 1, a M at a position corresponding to I16 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a M at position I16 of SEQ ID NO: 1, a Y at position P90 of SEQ ID NO: 1, and an I at position V93 of SEQ ID NO: 1. In some embodiments, a KOD polymerase variant comprises: a P at position Y7 of SEQ ID NO: 1, a M at position I16 of SEQ ID NO: 1, a Y at position P90 of SEQ ID NO: 1, and an I at position V93 of SEQ ID NO: 1.

### Y7P_I16M_V93Q

In some embodiments, a KOD polymerase variant comprises: a P at a position corresponding to Y7 of SEQ ID NO: 1, a M at a position corresponding to I16 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a M at position I16 of SEQ ID NO: 1, and a Q at position V93 of SEQ ID NO: 1. In some embodiments, a KOD polymerase variant comprises: a P at position Y7 of SEQ ID NO: 1, a M at position I16 of SEQ ID NO: 1, and a Q at position V93 of SEQ ID NO: 1.

### Y7P_P36A_P90W_V93I

In some embodiments, a KOD polymerase variant comprises: a P at a position corresponding to Y7 of SEQ ID NO: 1, an A at a position corresponding to P36 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, an A at position P36 of SEQ ID NO: 1, a W at position P90 of SEQ ID NO: 1, and an I at position V93 of SEQ ID NO: 1. In some embodiments, a KOD polymerase variant comprises: a P at position Y7 of SEQ ID NO: 1, an A at position P36 of SEQ ID NO: 1, a W at position P90 of SEQ ID NO: 1, and an I at position V93 of SEQ ID NO: 1.

### Y7P_P90F

In some embodiments, a KOD polymerase variant comprises: a P at a position corresponding to Y7 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, and a F at position P90 of SEQ ID NO: 1. In some embodiments, a KOD polymerase variant comprises: a P at position Y7 of SEQ ID NO: 1, and a F at position P90 of SEQ ID NO: 1.

### Y7P_P90W_V93I

In some embodiments, a KOD polymerase variant comprises: a P at a position corresponding to Y7 of SEQ ID NO: 1, a W at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a W at position P90 of SEQ ID NO: 1, and an I at position V93 of SEQ ID NO: 1. In some embodiments, a KOD polymerase variant comprises: a P at position Y7 of SEQ ID NO: 1, a W at position P90 of SEQ ID NO: 1, and an I at position V93 of SEQ ID NO: 1.

### Y7P_P90W_V93I_R97A

In some embodiments, a KOD polymerase variant comprises: a P at a position corresponding to Y7 of SEQ ID NO: 1, a W at a position corresponding to P90 of SEQ ID NO: 1, an I at a position corresponding to V93 of SEQ ID NO: 1, and an A at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a W at position P90 of SEQ ID NO: 1, an I at position V93 of SEQ ID NO: 1, and an A at position R97 of SEQ ID NO: 1. In some embodiments, a KOD polymerase variant comprises: a P at position Y7 of SEQ ID NO: 1, a W at position P90 of SEQ ID NO: 1, an I at position V93 of SEQ ID NO: 1, and an A at position R97 of SEQ ID NO: 1.

### Y7P_P90W_V93Q

In some embodiments, a KOD polymerase variant comprises: a P at a position corresponding to Y7 of SEQ ID NO: 1, a W at a position corresponding to P90 of SEQ ID NO: 1, and a Q at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a W at position P90 of SEQ ID NO: 1, and a Q at position V93 of SEQ ID NO: 1. In some embodiments, a KOD polymerase variant comprises: a P at position Y7 of SEQ ID NO: 1, a W at position P90 of SEQ ID NO: 1, and a Q at position V93 of SEQ ID NO: 1.

### Y7P_P90Y_V93I

In some embodiments, a KOD polymerase variant comprises: a P at a position corresponding to Y7 of SEQ ID NO: 1, a Y at a position corresponding to P90 of SEQ ID NO: 1, and an I at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position Y7 of SEQ ID NO: 1, a Y at position P90 of SEQ ID NO: 1, and an I at position V93 of SEQ ID NO: 1. In some embodiments, a KOD polymerase variant comprises: a P at position Y7 of SEQ ID NO: 1, a Y at position P90 of SEQ ID NO: 1, and an I at position V93 of SEQ ID NO: 1.

### 15p2s Assay Variants

### A117M

In some embodiments, a KOD polymerase variant comprises: a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position A117 of SEQ ID NO: 1. In some embodiments, a KOD polymerase variant comprises: a M at position A117 of SEQ ID NO: 1.

### A117M_D123I

In some embodiments, a KOD polymerase variant comprises: a M at a position corresponding to A117 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position A117 of SEQ ID NO: 1, and an I at position D123 of SEQ ID NO: 1. In some embodiments, a KOD polymerase variant comprises: a M at position A117 of SEQ ID NO: 1, and an I at position D123 of SEQ ID NO: 1.

### A117M_R119Q

In some embodiments, a KOD polymerase variant comprises: a M at a position corresponding to A117 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position A117 of SEQ ID NO: 1, and a Q at position R119 of SEQ ID NO: 1. In some embodiments, a KOD polymerase variant comprises: a M at position A117 of SEQ ID NO: 1, and a Q at position R119 of SEQ ID NO: 1.

### A117M_R119Q_D123I

In some embodiments, a KOD polymerase variant comprises: a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a M at position A117 of SEQ ID NO: 1, a Q at position R119 of SEQ ID NO: 1, and an I at position D123 of SEQ ID NO: 1. In some embodiments, a KOD polymerase variant comprises: a M at position A117 of SEQ ID NO: 1, a Q at position R119 of SEQ ID NO: 1, and an I at position D123 of SEQ ID NO: 1.

### F116P

In some embodiments, a KOD polymerase variant comprises: a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1. In some embodiments, a KOD polymerase variant comprises: a P at position F116 of SEQ ID NO: 1.

### F116P_A117M

In some embodiments, a KOD polymerase variant comprises: a P at a position corresponding to F116 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, and a M at position A117 of SEQ ID NO: 1. In some embodiments, a KOD polymerase variant comprises: a P at position F116 of SEQ ID NO: 1, and a M at position A117 of SEQ ID NO: 1.

### F116P_A117M_D123I

In some embodiments, a KOD polymerase variant comprises: a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, a M at position A117 of SEQ ID NO: 1, and an I at position D123 of SEQ ID NO: 1. In some embodiments, a KOD polymerase variant comprises: a P at position F116 of SEQ ID NO: 1, a M at position A117 of SEQ ID NO: 1, and an I at position D123 of SEQ ID NO: 1.

### F116P_A117M_R119Q

In some embodiments, a KOD polymerase variant comprises: a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, a M at position A117 of SEQ ID NO: 1, and a Q at position R119 of SEQ ID NO: 1. In some embodiments, a KOD polymerase variant comprises: a P at position F116 of SEQ ID NO: 1, a M at position A117 of SEQ ID NO: 1, and a Q at position R119 of SEQ ID NO: 1.

### F116P_A117M_R119Q_D123I

In some embodiments, a KOD polymerase variant comprises: a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a P at position F116 of SEQ ID NO: 1, a M at position A117 of SEQ ID NO: 1, a Q at position R119 of SEQ ID NO: 1, and an I at position D123 of SEQ ID NO: 1.

### F116P_D123I

In some embodiments, a KOD polymerase variant comprises: a P at a position corresponding to F116 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, and an I at position D123 of SEQ ID NO: 1. In some embodiments, a KOD polymerase variant comprises: a P at position F116 of SEQ ID NO: 1, and an I at position D123 of SEQ ID NO: 1.

### F116P_R119Q_D123I

In some embodiments, a KOD polymerase variant comprises: a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a P at position F116 of SEQ ID NO: 1, a Q at position R119 of SEQ ID NO: 1, and an I at position D123 of SEQ ID NO: 1. In some embodiments, a KOD polymerase variant comprises: a P at position F116 of SEQ ID NO: 1, a Q at position R119 of SEQ ID NO: 1, and an I at position D123 of SEQ ID NO: 1.

### H89D_A117M

In some embodiments, a KOD polymerase variant comprises: a D at a position corresponding to H89 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, and a M at position A117 of SEQ ID NO: 1. In some embodiments, a KOD polymerase variant comprises: a D at position H89 of SEQ ID NO: 1, and a M at position A117 of SEQ ID NO: 1.

### H89D_A117M_D123I

In some embodiments, a KOD polymerase variant comprises: a D at a position corresponding to H89 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a M at position A117 of SEQ ID NO: 1, and an I at position D123 of SEQ ID NO: 1. In some embodiments, a KOD polymerase variant comprises: a D at position H89 of SEQ ID NO: 1, a M at position A117 of SEQ ID NO: 1, and an I at position D123 of SEQ ID NO: 1.

### H89D_F116P

In some embodiments, a KOD polymerase variant comprises: a D at a position corresponding to H89 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, and a P at position F116 of SEQ ID NO: 1. In some embodiments, a KOD polymerase variant comprises: a D at position H89 of SEQ ID NO: 1, and a P at position F116 of SEQ ID NO: 1.

### H89D_F116P_A117M

In some embodiments, a KOD polymerase variant comprises: a D at a position corresponding to H89 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a P at position F116 of SEQ ID NO: 1, and a M at position A117 of SEQ ID NO: 1. In some embodiments, a KOD polymerase variant comprises: a D at position H89 of SEQ ID NO: 1, a P at position F116 of SEQ ID NO: 1, and a M at position A117 of SEQ ID NO: 1.

### H89D_F116P_D123I

In some embodiments, a KOD polymerase variant comprises: a D at a position corresponding to H89 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a P at position F116 of SEQ ID NO: 1, and an I at position D123 of SEQ ID NO: 1. In some embodiments, a KOD polymerase variant comprises: a D at position H89 of SEQ ID NO: 1, a P at position F116 of SEQ ID NO: 1, and an I at position D123 of SEQ ID NO: 1.

### H89D_P90F

In some embodiments, a KOD polymerase variant comprises: a D at a position corresponding to H89 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, and a F at position P90 of SEQ ID NO: 1. In some embodiments, a KOD polymerase variant comprises: a D at position H89 of SEQ ID NO: 1, and a F at position P90 of SEQ ID NO: 1.

### H89D_P90F_A117M

In some embodiments, a KOD polymerase variant comprises: a D at a position corresponding to H89 of SEQ ID NO: 1, a F at a position corresponding to P90 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, and a M at position A117 of SEQ ID NO: 1.

### H89D_P90F_A117M_D123I

In some embodiments, a KOD polymerase variant comprises: a D at a position corresponding to H89 of SEQ ID NO: 1, a F at a position corresponding to P90 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a M at position A117 of SEQ ID NO: 1, and an I at position D123 of SEQ ID NO: 1. In some embodiments, a KOD polymerase variant comprises: a D at position H89 of SEQ ID NO: 1, a F at position P90 of SEQ ID NO: 1, a M at position A117 of SEQ ID NO: 1, and an I at position D123 of SEQ ID NO: 1.

### H89D_P90F_F116P

In some embodiments, a KOD polymerase variant comprises: a D at a position corresponding to H89 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, and a P at position F116 of SEQ ID NO: 1. In some embodiments, a KOD polymerase variant comprises: a D at position H89 of SEQ ID NO: 1, a F at position P90 of SEQ ID NO: 1, and a P at position F116 of SEQ ID NO: 1.

### H89D_P90F_F116P_A117M

In some embodiments, a KOD polymerase variant comprises: a D at a position corresponding to H89 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, a P at position F116 of SEQ ID NO: 1, and a M at position A117 of SEQ ID NO: 1.

### H89D_P90F_F116P_A117M_D123I

In some embodiments, a KOD polymerase variant comprises: a D at a position corresponding to H89 of SEQ ID NO: 1, a F at a position corresponding to P90 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a D at position H89 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1.

### H89D_P90F_R97F_A117M

In some embodiments, a KOD polymerase variant comprises: a D at a position corresponding to H89 of SEQ ID NO: 1, a F at a position corresponding to P90 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a F at position R97 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, a F at position R97 of SEQ ID NO: 1, and a M at position A117 of SEQ ID NO: 1.

### H89D_P90F_R97F_F116P_A117M

In some embodiments, a KOD polymerase variant comprises: a D at a position corresponding to H89 of SEQ ID NO: 1, a F at a position corresponding to P90 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1.

### H89D_P90F_V93K_F116P

In some embodiments, a KOD polymerase variant comprises: a D at a position corresponding to H89 of SEQ ID NO: 1, a F at a position corresponding to P90 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a K at position V93 of SEQ ID NO: 1, and a P at position F116 of SEQ ID NO: 1. In some embodiments, a KOD polymerase variant comprises: a D at position H89 of SEQ ID NO: 1, a F at position P90 of SEQ ID NO: 1, a K at position V93 of SEQ ID NO: 1, and a P at position F116 of SEQ ID NO: 1.

### H89D_P90F_V93K_F116P_A117M

In some embodiments, a KOD polymerase variant comprises: a D at a position corresponding to H89 of SEQ ID NO: 1, a F at a position corresponding to P90 of SEQ ID NO: 1, a K at a position corresponding to V93 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a K at position V93 of SEQ ID NO: 1, a P at position F116 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, a K at position V93 of SEQ ID NO: 1, a P at position F116 of SEQ ID NO: 1, and a M at position A117 of SEQ ID NO: 1.

### H89D_P90F_V93K_P94D_F116P_A117M

In some embodiments, a KOD polymerase variant comprises: a D at a position corresponding to H89 of SEQ ID NO: 1, a F at a position corresponding to P90 of SEQ ID NO: 1, a K at a position corresponding to V93 of SEQ ID NO: 1, a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position P90 of SEQ ID NO: 1, a K at position V93 of SEQ ID NO: 1, a D at position P94 of SEQ ID NO: 1, a P at position F116 of SEQ ID NO: 1, and a M at position A117 of SEQ ID NO: 1. In some embodiments, a KOD polymerase variant comprises: a D at position H89 of SEQ ID NO: 1, a F at position P90 of SEQ ID NO: 1, a K at position V93 of SEQ ID NO: 1, a D at position P94 of SEQ ID NO: 1, a P at position F116 of SEQ ID NO: 1, and a M at position A117 of SEQ ID NO: 1.

### H89D_R97F_A117M

In some embodiments, a KOD polymerase variant comprises: a D at a position corresponding to H89 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a F at position R97 of SEQ ID NO: 1, and a M at position A117 of SEQ ID NO: 1. In some embodiments, a KOD polymerase variant comprises: a D at position H89 of SEQ ID NO: 1, a F at position R97 of SEQ ID NO: 1, and a M at position A117 of SEQ ID NO: 1.

### H89D_V93K

In some embodiments, a KOD polymerase variant comprises: a D at a position corresponding to H89 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, and a K at position V93 of SEQ ID NO: 1. In some embodiments, a KOD polymerase variant comprises: a D at position H89 of SEQ ID NO: 1, and a K at position V93 of SEQ ID NO: 1.

### H89D_V93K_A117M

In some embodiments, a KOD polymerase variant comprises: a D at a position corresponding to H89 of SEQ ID NO: 1, a K at a position corresponding to V93 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a K at position V93 of SEQ ID NO: 1, and a M at position A117 of SEQ ID NO: 1. In some embodiments, a KOD polymerase variant comprises: a D at position H89 of SEQ ID NO: 1, a K at position V93 of SEQ ID NO: 1, and a M at position A117 of SEQ ID NO: 1.

### H89D_V93K_P94D_A117M

In some embodiments, a KOD polymerase variant comprises: a D at a position corresponding to H89 of SEQ ID NO: 1, a K at a position corresponding to V93 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position H89 of SEQ ID NO: 1, a K at position V93 of SEQ ID NO: 1, a D at position P94 of SEQ ID NO: 1, and a M at position A117 of SEQ ID NO: 1. In some embodiments, a KOD polymerase variant comprises: a D at position H89 of SEQ ID NO: 1, a K at position V93 of SEQ ID NO: 1, a D at position P94 of SEQ ID NO: 1, and a M at position A117 of SEQ ID NO: 1.

### I16W

In some embodiments, a KOD polymerase variant comprises: a W at a position corresponding to I16 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1. In some embodiments, a KOD polymerase variant comprises: a W at position I16 of SEQ ID NO: 1.

### I16W_A117M

In some embodiments, a KOD polymerase variant comprises: a W at a position corresponding to I16 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a M at position A117 of SEQ ID NO: 1. In some embodiments, a KOD polymerase variant comprises: a W at position I16 of SEQ ID NO: 1, and a M at position A117 of SEQ ID NO: 1.

### I16W_A117M_D123I

In some embodiments, a KOD polymerase variant comprises: a W at a position corresponding to I16 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a M at position A117 of SEQ ID NO: 1, and an I at position D123 of SEQ ID NO: 1. In some embodiments, a KOD polymerase variant comprises: a W at position I16 of SEQ ID NO: 1, a M at position A117 of SEQ ID NO: 1, and an I at position D123 of SEQ ID NO: 1.

### I16W_D123I

In some embodiments, a KOD polymerase variant comprises: a W at a position corresponding to I16 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and an I at position D123 of SEQ ID NO: 1. In some embodiments, a KOD polymerase variant comprises: a W at position I16 of SEQ ID NO: 1, and an I at position D123 of SEQ ID NO: 1.

### I16W_F116P

In some embodiments, a KOD polymerase variant comprises: a W at a position corresponding to I16 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a P at position F116 of SEQ ID NO: 1. In some embodiments, a KOD polymerase variant comprises: a W at position I16 of SEQ ID NO: 1, and a P at position F116 of SEQ ID NO: 1.

### I16W_F116P_A117M

In some embodiments, a KOD polymerase variant comprises: a W at a position corresponding to I16 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a P at position F116 of SEQ ID NO: 1, and a M at position A117 of SEQ ID NO: 1.

### I16W_F116P_A117M_D123I

In some embodiments, a KOD polymerase variant comprises: a W at a position corresponding to I16 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a W at position I16 of SEQ ID NO: 1, a P at position F116 of SEQ ID NO: 1, a M at position A117 of SEQ ID NO: 1, and an I at position D123 of SEQ ID NO: 1.

### I16W_F116P_A117M_R119Q

In some embodiments, a KOD polymerase variant comprises: a W at a position corresponding to I16 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a W at position I16 of SEQ ID NO: 1, a P at position F116 of SEQ ID NO: 1, a M at position A117 of SEQ ID NO: 1, and a Q at position R119 of SEQ ID NO: 1.

### I16W_F116P_A117M_R119Q_D123I

In some embodiments, a KOD polymerase variant comprises: a W at a position corresponding to I16 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a W at position I16 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### I16W_F116P_D123I

In some embodiments, a KOD polymerase variant comprises: a W at a position corresponding to I16 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a P at position F116 of SEQ ID NO: 1, and an I at position D123 of SEQ ID NO: 1.

### I16W_F116P_R119Q

In some embodiments, a KOD polymerase variant comprises: a W at a position corresponding to I16 of SEQ ID NO: 1, a P at a position corresponding to F116 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, and a Q at position R119 of SEQ ID NO: 1. In some embodiments, a KOD polymerase variant comprises: a W at position I16 of SEQ ID NO: 1, a P at position F116 of SEQ ID NO: 1, and a Q at position R119 of SEQ ID NO: 1.

### I16W_F116P_R119Q_D123I

In some embodiments, a KOD polymerase variant comprises: a W at a position corresponding to I16 of SEQ ID NO: 1, a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a P at position F116 of SEQ ID NO: 1, a Q at position R119 of SEQ ID NO: 1, and an I at position D123 of SEQ ID NO: 1. In some embodiments, a KOD polymerase variant comprises: a W at position I16 of SEQ ID NO: 1, a P at position F116 of SEQ ID NO: 1, a Q at position R119 of SEQ ID NO: 1, and an I at position D123 of SEQ ID NO: 1.

### I16W_H89D

In some embodiments, a KOD polymerase variant comprises: a W at a position corresponding to I16 of SEQ ID NO: 1 and a D at a position corresponding to H89 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a D at position H89 of SEQ ID NO: 1. In some embodiments, a KOD polymerase variant comprises: a W at position I16 of SEQ ID NO: 1, and a D at position H89 of SEQ ID NO: 1.

### I16W_H89D_F116P

In some embodiments, a KOD polymerase variant comprises: a W at a position corresponding to I16 of SEQ ID NO: 1, a D at a position corresponding to H89 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position H89 of SEQ ID NO: 1, and a P at position F116 of SEQ ID NO: 1. In some embodiments, a KOD polymerase variant comprises: a W at position I16 of SEQ ID NO: 1, a D at position H89 of SEQ ID NO: 1, and a P at position F116 of SEQ ID NO: 1.

### I16W_H89D_F116P_A117M

In some embodiments, a KOD polymerase variant comprises: a W at a position corresponding to I16 of SEQ ID NO: 1, a D at a position corresponding to H89 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position H89 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a D at position H89 of SEQ ID NO: 1, a P at position F116 of SEQ ID NO: 1, and a M at position A117 of SEQ ID NO: 1.

### I16W_H89D_P90F_F116P

In some embodiments, a KOD polymerase variant comprises: a W at a position corresponding to I16 of SEQ ID NO: 1, a D at a position corresponding to H89 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position H89 of SEQ ID NO: 1, a F at position P90 of SEQ ID NO: 1, and a P at position F116 of SEQ ID NO: 1. In some embodiments, a KOD polymerase variant comprises: a W at position I16 of SEQ ID NO: 1, a D at position H89 of SEQ ID NO: 1, a F at position P90 of SEQ ID NO: 1, and a P at position F116 of SEQ ID NO: 1.

### I16W_P36H

In some embodiments, a KOD polymerase variant comprises: a W at a position corresponding to I16 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a H at position P36 of SEQ ID NO: 1. In some embodiments, a KOD polymerase variant comprises: a W at position I16 of SEQ ID NO: 1, and a H at position P36 of SEQ ID NO: 1.

### I16W_P36H_F116P

In some embodiments, a KOD polymerase variant comprises: a W at a position corresponding to I16 of SEQ ID NO: 1, a H at a position corresponding to P36 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, and a P at position F116 of SEQ ID NO: 1.

### I16W_P36H_F116P_A117M

In some embodiments, a KOD polymerase variant comprises: a W at a position corresponding to I16 of SEQ ID NO: 1, a H at a position corresponding to P36 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a H at position P36 of SEQ ID NO: 1, a P at position F116 of SEQ ID NO: 1, and a M at position A117 of SEQ ID NO: 1.

### I16W_P36H_F116P_D123I

In some embodiments, a KOD polymerase variant comprises: a W at a position corresponding to I16 of SEQ ID NO: 1, a H at a position corresponding to P36 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a H at position P36 of SEQ ID NO: 1, a P at position F116 of SEQ ID NO: 1, and an I at position D123 of SEQ ID NO: 1.

### I16W_P36H_H89D_F116P

In some embodiments, a KOD polymerase variant comprises: a W at a position corresponding to I16 of SEQ ID NO: 1, a H at a position corresponding to P36 of SEQ ID NO: 1, a D at a position corresponding to H89 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a D at position H89 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a D at position H89 of SEQ ID NO: 1, and a P at position F116 of SEQ ID NO: 1.

### I16W_P36H_H89D_F116P_A117M

In some embodiments, a KOD polymerase variant comprises: a W at a position corresponding to I16 of SEQ ID NO: 1, a H at a position corresponding to P36 of SEQ ID NO: 1, a D at a position corresponding to H89 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a D at position H89 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a H at position P36 of SEQ ID NO: 1, a D at position H89 of SEQ ID NO: 1, a P at position F116 of SEQ ID NO: 1, and a M at position A117 of SEQ ID NO: 1.

### I16W_P36H_P90F_F116P

In some embodiments, a KOD polymerase variant comprises: a W at a position corresponding to I16 of SEQ ID NO: 1, a H at a position corresponding to P36 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a F at position P90 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a F at position P90 of SEQ ID NO: 1, and a P at position F116 of SEQ ID NO: 1.

### I16W_P36H_P94D_F116P

In some embodiments, a KOD polymerase variant comprises: a W at a position corresponding to I16 of SEQ ID NO: 1, a H at a position corresponding to P36 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a D at position P94 of SEQ ID NO: 1, and a P at position F116 of SEQ ID NO: 1.

### I16W_P36H_R97F_F116P

In some embodiments, a KOD polymerase variant comprises: a W at a position corresponding to I16 of SEQ ID NO: 1, a H at a position corresponding to P36 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a F at position R97 of SEQ ID NO: 1, and a P at position F116 of SEQ ID NO: 1.

### I16W_P36H_R97F_F116P_A117M

In some embodiments, a KOD polymerase variant comprises: a W at a position corresponding to I16 of SEQ ID NO: 1, a H at a position corresponding to P36 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a H at position P36 of SEQ ID NO: 1, a F at position R97 of SEQ ID NO: 1, a P at position F116 of SEQ ID NO: 1, and a M at position A117 of SEQ ID NO: 1.

### I16W_P36H_V93K_F116P

In some embodiments, a KOD polymerase variant comprises: a W at a position corresponding to I16 of SEQ ID NO: 1, a H at a position corresponding to P36 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a K at position V93 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a K at position V93 of SEQ ID NO: 1, and a P at position F116 of SEQ ID NO: 1.

### I16W_P36H_V93K_P94D_F116P

In some embodiments, a KOD polymerase variant comprises: a W at a position corresponding to I16 of SEQ ID NO: 1, a H at a position corresponding to P36 of SEQ ID NO: 1, a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a K at position V93 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a K at position V93 of SEQ ID NO: 1, a D at position P94 of SEQ ID NO: 1, and a P at position F116 of SEQ ID NO: 1.

### I16W_P36H_V93K_R97F_F116P

In some embodiments, a KOD polymerase variant comprises: a W at a position corresponding to I16 of SEQ ID NO: 1, a H at a position corresponding to P36 of SEQ ID NO: 1, a K at a position corresponding to V93 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a H at position P36 of SEQ ID NO: 1, a K at position V93 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a H at position P36 of SEQ ID NO: 1, a K at position V93 of SEQ ID NO: 1, a F at position R97 of SEQ ID NO: 1, and a P at position F116 of SEQ ID NO: 1.

### I16W_P90F

In some embodiments, a KOD polymerase variant comprises: a W at a position corresponding to I16 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a F at position P90 of SEQ ID NO: 1. In some embodiments, a KOD polymerase variant comprises: a W at position I16 of SEQ ID NO: 1, and a F at position P90 of SEQ ID NO: 1.

### I16W_P90F_A117M

In some embodiments, a KOD polymerase variant comprises: a W at a position corresponding to I16 of SEQ ID NO: 1, a F at a position corresponding to P90 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, and a M at position A117 of SEQ ID NO: 1. In some embodiments, a KOD polymerase variant comprises: a W at position I16 of SEQ ID NO: 1, a F at position P90 of SEQ ID NO: 1, and a M at position A117 of SEQ ID NO: 1.

### I16W_P90F_F116P

In some embodiments, a KOD polymerase variant comprises: a W at a position corresponding to I16 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, and a P at position F116 of SEQ ID NO: 1.

### I16W_P90F_F116P_A117M

In some embodiments, a KOD polymerase variant comprises: a W at a position corresponding to I16 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a F at position P90 of SEQ ID NO: 1, a P at position F116 of SEQ ID NO: 1, and a M at position A117 of SEQ ID NO: 1.

### I16W_P90F_F116P_D123I

In some embodiments, a KOD polymerase variant comprises: a W at a position corresponding to I16 of SEQ ID NO: 1, a F at a position corresponding to P90 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a F at position P90 of SEQ ID NO: 1, a P at position F116 of SEQ ID NO: 1, and an I at position D123 of SEQ ID NO: 1.

### I16W_P90F_P94D_F116P

In some embodiments, a KOD polymerase variant comprises: a W at a position corresponding to I16 of SEQ ID NO: 1, a F at a position corresponding to P90 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a D at position P94 of SEQ ID NO: 1, and a P at position F116 of SEQ ID NO: 1.

### I16W_P90F_P94D_R97F_F116P

In some embodiments, a KOD polymerase variant comprises: a W at a position corresponding to I16 of SEQ ID NO: 1, a F at a position corresponding to P90 of SEQ ID NO: 1, a D at a position corresponding to P94 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a D at position P94 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a D at position P94 of SEQ ID NO: 1, a F at position R97 of SEQ ID NO: 1, and a P at position F116 of SEQ ID NO: 1.

### I16W_P90F_R97F_F116P

In some embodiments, a KOD polymerase variant comprises: a W at a position corresponding to I16 of SEQ ID NO: 1, a F at a position corresponding to P90 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a F at position R97 of SEQ ID NO: 1, and a P at position F116 of SEQ ID NO: 1.

### I16W_P90F_R97F_F116P_A117M

In some embodiments, a KOD polymerase variant comprises: a W at a position corresponding to I16 of SEQ ID NO: 1, a F at a position corresponding to P90 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1.

### I16W_P90F_V93K_F116P

In some embodiments, a KOD polymerase variant comprises: a W at a position corresponding to I16 of SEQ ID NO: 1, a F at a position corresponding to P90 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a K at position V93 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a K at position V93 of SEQ ID NO: 1, and a P at position F116 of SEQ ID NO: 1.

### I16W_P90F_V93K_P94D_F116P

In some embodiments, a KOD polymerase variant comprises: a W at a position corresponding to I16 of SEQ ID NO: 1, a F at a position corresponding to P90 of SEQ ID NO: 1, a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a K at position V93 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a K at position V93 of SEQ ID NO: 1, a D at position P94 of SEQ ID NO: 1, and a P at position F116 of SEQ ID NO: 1.

### I16W_P90F_V93K_P94D_R97F_F116P

In some embodiments, a KOD polymerase variant comprises: a W at a position corresponding to I16 of SEQ ID NO: 1, a F at a position corresponding to P90 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1.

### I16W_P90F_V93K_R97F_F116P

In some embodiments, a KOD polymerase variant comprises: a W at a position corresponding to I16 of SEQ ID NO: 1, a F at a position corresponding to P90 of SEQ ID NO: 1, a K at a position corresponding to V93 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position P90 of SEQ ID NO: 1, a K at position V93 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a F at position P90 of SEQ ID NO: 1, a K at position V93 of SEQ ID NO: 1, a F at position R97 of SEQ ID NO: 1, and a P at position F116 of SEQ ID NO: 1.

### I16W_P94D

In some embodiments, a KOD polymerase variant comprises: a W at a position corresponding to I16 of SEQ ID NO: 1 and a D at a position corresponding to P94 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a D at position P94 of SEQ ID NO: 1. In some embodiments, a KOD polymerase variant comprises: a W at position I16 of SEQ ID NO: 1, and a D at position P94 of SEQ ID NO: 1.

### I16W_P94D_F116P

In some embodiments, a KOD polymerase variant comprises: a W at a position corresponding to I16 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a D at position P94 of SEQ ID NO: 1, and a P at position F116 of SEQ ID NO: 1.

### I16W_P94D_F116P_A117M

In some embodiments, a KOD polymerase variant comprises: a W at a position corresponding to I16 of SEQ ID NO: 1, a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position P94 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a D at position P94 of SEQ ID NO: 1, a P at position F116 of SEQ ID NO: 1, and a M at position A117 of SEQ ID NO: 1.

### I16W_P94D_F116P_D123I

In some embodiments, a KOD polymerase variant comprises: a W at a position corresponding to I16 of SEQ ID NO: 1, a D at a position corresponding to P94 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position P94 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a D at position P94 of SEQ ID NO: 1, a P at position F116 of SEQ ID NO: 1, and an I at position D123 of SEQ ID NO: 1.

### I16W_R97F

In some embodiments, a KOD polymerase variant comprises: a W at a position corresponding to I16 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, and a F at position R97 of SEQ ID NO: 1. In some embodiments, a KOD polymerase variant comprises: a W at position I16 of SEQ ID NO: 1, and a F at position R97 of SEQ ID NO: 1.

### I16W_R97F_A117M

In some embodiments, a KOD polymerase variant comprises: a W at a position corresponding to I16 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, and a M at position A117 of SEQ ID NO: 1. In some embodiments, a KOD polymerase variant comprises: a W at position I16 of SEQ ID NO: 1, a F at position R97 of SEQ ID NO: 1, and a M at position A117 of SEQ ID NO: 1.

### I16W_R97F_D123I

In some embodiments, a KOD polymerase variant comprises: a W at a position corresponding to I16 of SEQ ID NO: 1, a F at a position corresponding to R97 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, and an I at position D123 of SEQ ID NO: 1. In some embodiments, a KOD polymerase variant comprises: a W at position I16 of SEQ ID NO: 1, a F at position R97 of SEQ ID NO: 1, and an I at position D123 of SEQ ID NO: 1.

### I16W_R97F_F116P

In some embodiments, a KOD polymerase variant comprises: a W at a position corresponding to I16 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a F at position R97 of SEQ ID NO: 1, and a P at position F116 of SEQ ID NO: 1.

### I16W_R97F_F116P_A117M

In some embodiments, a KOD polymerase variant comprises: a W at a position corresponding to I16 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a F at position R97 of SEQ ID NO: 1, a P at position F116 of SEQ ID NO: 1, and a M at position A117 of SEQ ID NO: 1.

### I16W_R97F_F116P_A117M_D123I

In some embodiments, a KOD polymerase variant comprises: a W at a position corresponding to I16 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a W at position I16 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1.

### I16W_R97F_F116P_D123I

In some embodiments, a KOD polymerase variant comprises: a W at a position corresponding to I16 of SEQ ID NO: 1, a F at a position corresponding to R97 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a F at position R97 of SEQ ID NO: 1, a P at position F116 of SEQ ID NO: 1, and an I at position D123 of SEQ ID NO: 1.

### I16W_R97F_F116P_R119Q_D123I

In some embodiments, a KOD polymerase variant comprises: a W at a position corresponding to I16 of SEQ ID NO: 1, a F at a position corresponding to R97 of SEQ ID NO: 1, a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a F at position R97 of SEQ ID NO: 1, a P at position F116 of SEQ ID NO: 1, a Q at position R119 of SEQ ID NO: 1, and an I at position D123 of SEQ ID NO: 1. In some embodiments, a KOD polymerase variant comprises: a W at position I16 of SEQ ID NO: 1, a F at position R97 of SEQ ID NO: 1, a P at position F116 of SEQ ID NO: 1, a Q at position R119 of SEQ ID NO: 1, and an I at position D123 of SEQ ID NO: 1.

### I16W_V93K_F116P

In some embodiments, a KOD polymerase variant comprises: a W at a position corresponding to I16 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, and a P at position F116 of SEQ ID NO: 1. In some embodiments, a KOD polymerase variant comprises: a W at position I16 of SEQ ID NO: 1, a K at position V93 of SEQ ID NO: 1, and a P at position F116 of SEQ ID NO: 1.

### I16W_V93K_F116P_A117M

In some embodiments, a KOD polymerase variant comprises: a W at a position corresponding to I16 of SEQ ID NO: 1, a K at a position corresponding to V93 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a K at position V93 of SEQ ID NO: 1, a P at position F116 of SEQ ID NO: 1, and a M at position A117 of SEQ ID NO: 1.

### I16W_V93K_F116P_A117M_R119Q

In some embodiments, a KOD polymerase variant comprises: a W at a position corresponding to I16 of SEQ ID NO: 1, a K at a position corresponding to V93 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a W at position I16 of SEQ ID NO: 1, a K at position V93 of SEQ ID NO: 1, a P at position F116 of SEQ ID NO: 1, a M at position A117 of SEQ ID NO: 1, and a Q at position R119 of SEQ ID NO: 1.

### I16W_V93K_F116P_R119Q

In some embodiments, a KOD polymerase variant comprises: a W at a position corresponding to I16 of SEQ ID NO: 1, a K at a position corresponding to V93 of SEQ ID NO: 1, a P at a position corresponding to F116 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a P at position F116 of SEQ ID NO: 1, and a Q at position R119 of SEQ ID NO: 1. In some embodiments, a KOD polymerase variant comprises: a W at position I16 of SEQ ID NO: 1, a K at position V93 of SEQ ID NO: 1, a P at position F116 of SEQ ID NO: 1, and a Q at position R119 of SEQ ID NO: 1.

### I16W_V93K_F116P_R119Q_D123I

In some embodiments, a KOD polymerase variant comprises: a W at a position corresponding to I16 of SEQ ID NO: 1, a K at a position corresponding to V93 of SEQ ID NO: 1, a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a P at position F116 of SEQ ID NO: 1, a Q at position R119 of SEQ ID NO: 1, and an I at position D123 of SEQ ID NO: 1. In some embodiments, a KOD polymerase variant comprises: a W at position I16 of SEQ ID NO: 1, a K at position V93 of SEQ ID NO: 1, a P at position F116 of SEQ ID NO: 1, a Q at position R119 of SEQ ID NO: 1, and an I at position D123 of SEQ ID NO: 1.

### I16W_V93K_P94D_F116P

In some embodiments, a KOD polymerase variant comprises: a W at a position corresponding to I16 of SEQ ID NO: 1, a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a D at position P94 of SEQ ID NO: 1, and a P at position F116 of SEQ ID NO: 1. In some embodiments, a KOD polymerase variant comprises: a W at position I16 of SEQ ID NO: 1, a K at position V93 of SEQ ID NO: 1, a D at position P94 of SEQ ID NO: 1, and a P at position F116 of SEQ ID NO: 1.

### I16W_V93K_P94D_R97F_F116P

In some embodiments, a KOD polymerase variant comprises: a W at a position corresponding to I16 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1.

### I16W_V93K_R97F_F116P

In some embodiments, a KOD polymerase variant comprises: a W at a position corresponding to I16 of SEQ ID NO: 1, a K at a position corresponding to V93 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a F at position R97 of SEQ ID NO: 1, and a P at position F116 of SEQ ID NO: 1. In some embodiments, a KOD polymerase variant comprises: a W at position I16 of SEQ ID NO: 1, a K at position V93 of SEQ ID NO: 1, a F at position R97 of SEQ ID NO: 1, and a P at position F116 of SEQ ID NO: 1.

### I16W_V93K_R97F_F116P_A117M

In some embodiments, a KOD polymerase variant comprises: a W at a position corresponding to I16 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a W at position I16 of SEQ ID NO: 1, a K at position V93 of SEQ ID NO: 1, a F at position R97 of SEQ ID NO: 1, a P at position F116 of SEQ ID NO: 1, and a M at position A117 of SEQ ID NO: 1.

### I16W_V93K_R97F_F116P_R119Q

In some embodiments, a KOD polymerase variant comprises: a W at a position corresponding to I16 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a K at position V93 of SEQ ID NO: 1, a F at position R97 of SEQ ID NO: 1, a P at position F116 of SEQ ID NO: 1, and a Q at position R119 of SEQ ID NO: 1. In some embodiments, a KOD polymerase variant comprises: a W at position I16 of SEQ ID NO: 1, a K at position V93 of SEQ ID NO: 1, a F at position R97 of SEQ ID NO: 1, a P at position F116 of SEQ ID NO: 1, and a Q at position R119 of SEQ ID NO: 1.

### I16W_Y37D_F116P_D123I

In some embodiments, a KOD polymerase variant comprises: a W at a position corresponding to I16 of SEQ ID NO: 1, a D at a position corresponding to Y37 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a W at position I16 of SEQ ID NO: 1, a D at position Y37 of SEQ ID NO: 1, a P at position F116 of SEQ ID NO: 1, and an I at position D123 of SEQ ID NO: 1. In some embodiments, a KOD polymerase variant comprises: a W at position I16 of SEQ ID NO: 1, a D at position Y37 of SEQ ID NO: 1, a P at position F116 of SEQ ID NO: 1, and an I at position D123 of SEQ ID NO: 1.

### P14E_I16W_P90F_F116P

In some embodiments, a KOD polymerase variant comprises: an E at a position corresponding to P14 of SEQ ID NO: 1, a W at a position corresponding to I16 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a W at position I16 of SEQ ID NO: 1, a F at position P90 of SEQ ID NO: 1, and a P at position F116 of SEQ ID NO: 1. In some embodiments, a KOD polymerase variant comprises: an E at position P14 of SEQ ID NO: 1, a W at position I16 of SEQ ID NO: 1, a F at position P90 of SEQ ID NO: 1, and a P at position F116 of SEQ ID NO: 1.

### P14E_P36H

In some embodiments, a KOD polymerase variant comprises: an E at a position corresponding to P14 of SEQ ID NO: 1 and a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, and a H at position P36 of SEQ ID NO: 1. In some embodiments, a KOD polymerase variant comprises: an E at position P14 of SEQ ID NO: 1, and a H at position P36 of SEQ ID NO: 1.

### P14E_P36H_P90F_F116P_A117M

In some embodiments, a KOD polymerase variant comprises: an E at a position corresponding to P14 of SEQ ID NO: 1, a H at a position corresponding to P36 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a H at position P36 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: an E at position P14 of SEQ ID NO: 1, a H at position P36 of SEQ ID NO: 1, a F at position P90 of SEQ ID NO: 1, a P at position F116 of SEQ ID NO: 1, and a M at position A117 of SEQ ID NO: 1.

### P14E_P90F

In some embodiments, a KOD polymerase variant comprises: an E at a position corresponding to P14 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, and a F at position P90 of SEQ ID NO: 1. In some embodiments, a KOD polymerase variant comprises: an E at position P14 of SEQ ID NO: 1, and a F at position P90 of SEQ ID NO: 1.

### P14E_P90F_A117M

In some embodiments, a KOD polymerase variant comprises: an E at a position corresponding to P14 of SEQ ID NO: 1, a F at a position corresponding to P90 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a F at position P90 of SEQ ID NO: 1, and a M at position A117 of SEQ ID NO: 1. In some embodiments, a KOD polymerase variant comprises: an E at position P14 of SEQ ID NO: 1, a F at position P90 of SEQ ID NO: 1, and a M at position A117 of SEQ ID NO: 1.

### P14E_P90F_F116P

In some embodiments, a KOD polymerase variant comprises: an E at a position corresponding to P14 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a F at position P90 of SEQ ID NO: 1, and a P at position F116 of SEQ ID NO: 1. In some embodiments, a KOD polymerase variant comprises: an E at position P14 of SEQ ID NO: 1, a F at position P90 of SEQ ID NO: 1, and a P at position F116 of SEQ ID NO: 1.

### P14E_P90F_F116P_A117M

In some embodiments, a KOD polymerase variant comprises: an E at a position corresponding to P14 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: an E at position P14 of SEQ ID NO: 1, a F at position P90 of SEQ ID NO: 1, a P at position F116 of SEQ ID NO: 1, and a M at position A117 of SEQ ID NO: 1.

### P14E_P90F_F116P_A117M_D123I

In some embodiments, a KOD polymerase variant comprises: an E at a position corresponding to P14 of SEQ ID NO: 1, a F at a position corresponding to P90 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and an E at position P14 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: an E at position P14 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1.

### P36H

In some embodiments, a KOD polymerase variant comprises: a H at a position corresponding to P36 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1. In some embodiments, a KOD polymerase variant comprises: a H at position P36 of SEQ ID NO: 1.

### P36H_A117M

In some embodiments, a KOD polymerase variant comprises: a H at a position corresponding to P36 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a M at position A117 of SEQ ID NO: 1. In some embodiments, a KOD polymerase variant comprises: a H at position P36 of SEQ ID NO: 1, and a M at position A117 of SEQ ID NO: 1.

### P36H_A117M_D123I

In some embodiments, a KOD polymerase variant comprises: a H at a position corresponding to P36 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a M at position A117 of SEQ ID NO: 1, and an I at position D123 of SEQ ID NO: 1.

### P36H_D123I

In some embodiments, a KOD polymerase variant comprises: a H at a position corresponding to P36 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and an I at position D123 of SEQ ID NO: 1. In some embodiments, a KOD polymerase variant comprises: a H at position P36 of SEQ ID NO: 1, and an I at position D123 of SEQ ID NO: 1.

### P36H_F116P

In some embodiments, a KOD polymerase variant comprises: a H at a position corresponding to P36 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a P at position F116 of SEQ ID NO: 1. In some embodiments, a KOD polymerase variant comprises: a H at position P36 of SEQ ID NO: 1, and a P at position F116 of SEQ ID NO: 1.

### P36H_F116P_A117M

In some embodiments, a KOD polymerase variant comprises: a H at a position corresponding to P36 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a P at position F116 of SEQ ID NO: 1, and a M at position A117 of SEQ ID NO: 1.

### P36H_F116P_A117M_D123I

In some embodiments, a KOD polymerase variant comprises: a H at a position corresponding to P36 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a P at position F116 of SEQ ID NO: 1, a M at position A117 of SEQ ID NO: 1, and an I at position D123 of SEQ ID NO: 1.

### P36H_F116P_D123I

In some embodiments, a KOD polymerase variant comprises: a H at a position corresponding to P36 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a P at position F116 of SEQ ID NO: 1, and an I at position D123 of SEQ ID NO: 1. In some embodiments, a KOD polymerase variant comprises: a H at position P36 of SEQ ID NO: 1, a P at position F116 of SEQ ID NO: 1, and an I at position D123 of SEQ ID NO: 1.

### P36H_H89D

In some embodiments, a KOD polymerase variant comprises: a H at a position corresponding to P36 of SEQ ID NO: 1 and a D at a position corresponding to H89 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a D at position H89 of SEQ ID NO: 1. In some embodiments, a KOD polymerase variant comprises: a H at position P36 of SEQ ID NO: 1, and a D at position H89 of SEQ ID NO: 1.

### P36H_H89D_A117M

In some embodiments, a KOD polymerase variant comprises: a H at a position corresponding to P36 of SEQ ID NO: 1, a D at a position corresponding to H89 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, and a M at position A117 of SEQ ID NO: 1. In some embodiments, a KOD polymerase variant comprises: a H at position P36 of SEQ ID NO: 1, a D at position H89 of SEQ ID NO: 1, and a M at position A117 of SEQ ID NO: 1.

### P36H_H89D_F116P

In some embodiments, a KOD polymerase variant comprises: a H at a position corresponding to P36 of SEQ ID NO: 1, a D at a position corresponding to H89 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, and a P at position F116 of SEQ ID NO: 1. In some embodiments, a KOD polymerase variant comprises: a H at position P36 of SEQ ID NO: 1, a D at position H89 of SEQ ID NO: 1, and a P at position F116 of SEQ ID NO: 1.

### P36H_H89D_F116P_A117M

In some embodiments, a KOD polymerase variant comprises: a H at a position corresponding to P36 of SEQ ID NO: 1, a D at a position corresponding to H89 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a D at position H89 of SEQ ID NO: 1, a P at position F116 of SEQ ID NO: 1, and a M at position A117 of SEQ ID NO: 1.

### P36H_H89D_P90F_A117M

In some embodiments, a KOD polymerase variant comprises: a H at a position corresponding to P36 of SEQ ID NO: 1, a D at a position corresponding to H89 of SEQ ID NO: 1, a F at a position corresponding to P90 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, a F at position P90 of SEQ ID NO: 1, and a M at position A117 of SEQ ID NO: 1. In some embodiments, a KOD polymerase variant comprises: a H at position P36 of SEQ ID NO: 1, a D at position H89 of SEQ ID NO: 1, a F at position P90 of SEQ ID NO: 1, and a M at position A117 of SEQ ID NO: 1.

### P36H_H89D_P90F_F116P

In some embodiments, a KOD polymerase variant comprises: a H at a position corresponding to P36 of SEQ ID NO: 1, a D at a position corresponding to H89 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, a F at position P90 of SEQ ID NO: 1, and a P at position F116 of SEQ ID NO: 1. In some embodiments, a KOD polymerase variant comprises: a H at position P36 of SEQ ID NO: 1, a D at position H89 of SEQ ID NO: 1, a F at position P90 of SEQ ID NO: 1, and a P at position F116 of SEQ ID NO: 1.

### P36H_H89D_P90F_F116P_A117M

In some embodiments, a KOD polymerase variant comprises: a H at a position corresponding to P36 of SEQ ID NO: 1, a D at a position corresponding to H89 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position H89 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a D at position H89 of SEQ ID NO: 1, a F at position P90 of SEQ ID NO: 1, a P at position F116 of SEQ ID NO: 1, and a M at position A117 of SEQ ID NO: 1.

### P36H_P90F

In some embodiments, a KOD polymerase variant comprises: a H at a position corresponding to P36 of SEQ ID NO: 1 and a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a F at position P90 of SEQ ID NO: 1. In some embodiments, a KOD polymerase variant comprises: a H at position P36 of SEQ ID NO: 1, and a F at position P90 of SEQ ID NO: 1.

### P36H_P90F_A117M

In some embodiments, a KOD polymerase variant comprises: a H at a position corresponding to P36 of SEQ ID NO: 1, a F at a position corresponding to P90 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, and a M at position A117 of SEQ ID NO: 1. In some embodiments, a KOD polymerase variant comprises: a H at position P36 of SEQ ID NO: 1, a F at position P90 of SEQ ID NO: 1, and a M at position A117 of SEQ ID NO: 1.

### P36H_P90F_A117M_D123I

In some embodiments, a KOD polymerase variant comprises: a H at a position corresponding to P36 of SEQ ID NO: 1, a F at a position corresponding to P90 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a F at position P90 of SEQ ID NO: 1, a M at position A117 of SEQ ID NO: 1, and an I at position D123 of SEQ ID NO: 1.

### P36H_P90F_F116P

In some embodiments, a KOD polymerase variant comprises: a H at a position corresponding to P36 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, and a P at position F116 of SEQ ID NO: 1. In some embodiments, a KOD polymerase variant comprises: a H at position P36 of SEQ ID NO: 1, a F at position P90 of SEQ ID NO: 1, and a P at position F116 of SEQ ID NO: 1.

### P36H_P90F_F116P_A117M

In some embodiments, a KOD polymerase variant comprises: a H at a position corresponding to P36 of SEQ ID NO: 1, a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a F at position P90 of SEQ ID NO: 1, a P at position F116 of SEQ ID NO: 1, and a M at position A117 of SEQ ID NO: 1.

### P36H_P90F_F116P_A117M_D123I

In some embodiments, a KOD polymerase variant comprises: a H at a position corresponding to P36 of SEQ ID NO: 1, a F at a position corresponding to P90 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1.

### P36H_P90F_P94D_A117M

In some embodiments, a KOD polymerase variant comprises: a H at a position corresponding to P36 of SEQ ID NO: 1, a F at a position corresponding to P90 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a D at position P94 of SEQ ID NO: 1, and a M at position A117 of SEQ ID NO: 1. In some embodiments, a KOD polymerase variant comprises: a H at position P36 of SEQ ID NO: 1, a F at position P90 of SEQ ID NO: 1, a D at position P94 of SEQ ID NO: 1, and a M at position A117 of SEQ ID NO: 1.

### P36H_P90F_R97F_A117M

In some embodiments, a KOD polymerase variant comprises: a H at a position corresponding to P36 of SEQ ID NO: 1, a F at a position corresponding to P90 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a F at position R97 of SEQ ID NO: 1, and a M at position A117 of SEQ ID NO: 1. In some embodiments, a KOD polymerase variant comprises: a H at position P36 of SEQ ID NO: 1, a F at position P90 of SEQ ID NO: 1, a F at position R97 of SEQ ID NO: 1, and a M at position A117 of SEQ ID NO: 1.

### P36H_P90F_R97F_A117M_D123I

In some embodiments, a KOD polymerase variant comprises: a H at a position corresponding to P36 of SEQ ID NO: 1, a F at a position corresponding to P90 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a F at position P90 of SEQ ID NO: 1, a F at position R97 of SEQ ID NO: 1, a M at position A117 of SEQ ID NO: 1, and an I at position D123 of SEQ ID NO: 1.

### P36H_P90F_R97F_F116P_A117M

In some embodiments, a KOD polymerase variant comprises: a H at a position corresponding to P36 of SEQ ID NO: 1, a F at a position corresponding to P90 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a F at position P90 of SEQ ID NO: 1, a F at position R97 of SEQ ID NO: 1, a P at position F116 of SEQ ID NO: 1, and a M at position A117 of SEQ ID NO: 1.

### P36H_P90F_R97F_F116P_A117M_D123I

In some embodiments, a KOD polymerase variant comprises: a H at a position corresponding to P36 of SEQ ID NO: 1, a F at a position corresponding to P90 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position P90 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a F at position P90 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1.

### P36H_P94D_A117M

In some embodiments, a KOD polymerase variant comprises: a H at a position corresponding to P36 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position P94 of SEQ ID NO: 1, and a M at position A117 of SEQ ID NO: 1. In some embodiments, a KOD polymerase variant comprises: a H at position P36 of SEQ ID NO: 1, a D at position P94 of SEQ ID NO: 1, and a M at position A117 of SEQ ID NO: 1.

### P36H_P94D_A117M_D123I

In some embodiments, a KOD polymerase variant comprises: a H at a position corresponding to P36 of SEQ ID NO: 1, a D at a position corresponding to P94 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a D at position P94 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a D at position P94 of SEQ ID NO: 1, a M at position A117 of SEQ ID NO: 1, and an I at position D123 of SEQ ID NO: 1.

### P36H_R97F

In some embodiments, a KOD polymerase variant comprises: a H at a position corresponding to P36 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a F at position R97 of SEQ ID NO: 1. In some embodiments, a KOD polymerase variant comprises: a H at position P36 of SEQ ID NO: 1, and a F at position R97 of SEQ ID NO: 1.

### P36H_R97F_A117M

In some embodiments, a KOD polymerase variant comprises: a H at a position corresponding to P36 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position R97 of SEQ ID NO: 1, and a M at position A117 of SEQ ID NO: 1. In some embodiments, a KOD polymerase variant comprises: a H at position P36 of SEQ ID NO: 1, a F at position R97 of SEQ ID NO: 1, and a M at position A117 of SEQ ID NO: 1.

### P36H_R97F_A117M_D123I

In some embodiments, a KOD polymerase variant comprises: a H at a position corresponding to P36 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a H at position P36 of SEQ ID NO: 1, a F at position R97 of SEQ ID NO: 1, a M at position A117 of SEQ ID NO: 1, and an I at position D123 of SEQ ID NO: 1.

### P36H_R97F_F116P

In some embodiments, a KOD polymerase variant comprises: a H at a position corresponding to P36 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position R97 of SEQ ID NO: 1, and a P at position F116 of SEQ ID NO: 1. In some embodiments, a KOD polymerase variant comprises: a H at position P36 of SEQ ID NO: 1, a F at position R97 of SEQ ID NO: 1, and a P at position F116 of SEQ ID NO: 1.

### P36H_R97F_F116P_A117M

In some embodiments, a KOD polymerase variant comprises: a H at a position corresponding to P36 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a H at position P36 of SEQ ID NO: 1, a F at position R97 of SEQ ID NO: 1, a P at position F116 of SEQ ID NO: 1, and a M at position A117 of SEQ ID NO: 1.

### P36H_V93K

In some embodiments, a KOD polymerase variant comprises: a H at a position corresponding to P36 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, and a K at position V93 of SEQ ID NO: 1. In some embodiments, a KOD polymerase variant comprises: a H at position P36 of SEQ ID NO: 1, and a K at position V93 of SEQ ID NO: 1.

### P36H_V93K_A117M

In some embodiments, a KOD polymerase variant comprises: a H at a position corresponding to P36 of SEQ ID NO: 1, a K at a position corresponding to V93 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, and a M at position A117 of SEQ ID NO: 1. In some embodiments, a KOD polymerase variant comprises: a H at position P36 of SEQ ID NO: 1, a K at position V93 of SEQ ID NO: 1, and a M at position A117 of SEQ ID NO: 1.

### P36H_V93K_F116P

In some embodiments, a KOD polymerase variant comprises: a H at a position corresponding to P36 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, and a P at position F116 of SEQ ID NO: 1. In some embodiments, a KOD polymerase variant comprises: a H at position P36 of SEQ ID NO: 1, a K at position V93 of SEQ ID NO: 1, and a P at position F116 of SEQ ID NO: 1.

### P36H_V93K_P94D

In some embodiments, a KOD polymerase variant comprises: a H at a position corresponding to P36 of SEQ ID NO: 1, a K at a position corresponding to V93 of SEQ ID NO: 1, and a D at a position corresponding to P94 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, and a D at position P94 of SEQ ID NO: 1. In some embodiments, a KOD polymerase variant comprises: a H at position P36 of SEQ ID NO: 1, a K at position V93 of SEQ ID NO: 1, and a D at position P94 of SEQ ID NO: 1.

### P36H_V93K_P94D_A117M

In some embodiments, a KOD polymerase variant comprises: a H at a position corresponding to P36 of SEQ ID NO: 1, a K at a position corresponding to V93 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, a D at position P94 of SEQ ID NO: 1, and a M at position A117 of SEQ ID NO: 1. In some embodiments, a KOD polymerase variant comprises: a H at position P36 of SEQ ID NO: 1, a K at position V93 of SEQ ID NO: 1, a D at position P94 of SEQ ID NO: 1, and a M at position A117 of SEQ ID NO: 1.

### P36H_V93K_P94D_F116P

In some embodiments, a KOD polymerase variant comprises: a H at a position corresponding to P36 of SEQ ID NO: 1, a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, a D at position P94 of SEQ ID NO: 1, and a P at position F116 of SEQ ID NO: 1. In some embodiments, a KOD polymerase variant comprises: a H at position P36 of SEQ ID NO: 1, a K at position V93 of SEQ ID NO: 1, a D at position P94 of SEQ ID NO: 1, and a P at position F116 of SEQ ID NO: 1.

### P36H_V93K_R97F_A117M

In some embodiments, a KOD polymerase variant comprises: a H at a position corresponding to P36 of SEQ ID NO: 1, a K at a position corresponding to V93 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a H at position P36 of SEQ ID NO: 1, a K at position V93 of SEQ ID NO: 1, a F at position R97 of SEQ ID NO: 1, and a M at position A117 of SEQ ID NO: 1. In some embodiments, a KOD polymerase variant comprises: a H at position P36 of SEQ ID NO: 1, a K at position V93 of SEQ ID NO: 1, a F at position R97 of SEQ ID NO: 1, and a M at position A117 of SEQ ID NO: 1.

### P90F

In some embodiments, a KOD polymerase variant comprises: a F at a position corresponding to P90 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1. In some embodiments, a KOD polymerase variant comprises: a F at position P90 of SEQ ID NO: 1.

### P90F_A117M

In some embodiments, a KOD polymerase variant comprises: a F at a position corresponding to P90 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a M at position A117 of SEQ ID NO: 1. In some embodiments, a KOD polymerase variant comprises: a F at position P90 of SEQ ID NO: 1, and a M at position A117 of SEQ ID NO: 1.

### P90F_A117M_D123I

In some embodiments, a KOD polymerase variant comprises: a F at a position corresponding to P90 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a M at position A117 of SEQ ID NO: 1, and an I at position D123 of SEQ ID NO: 1.

### P90F_A117M_R119Q

In some embodiments, a KOD polymerase variant comprises: a F at a position corresponding to P90 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a M at position A117 of SEQ ID NO: 1, and a Q at position R119 of SEQ ID NO: 1. In some embodiments, a KOD polymerase variant comprises: a F at position P90 of SEQ ID NO: 1, a M at position A117 of SEQ ID NO: 1, and a Q at position R119 of SEQ ID NO: 1.

### P90F_A117M_R119Q_D123I

In some embodiments, a KOD polymerase variant comprises: a F at a position corresponding to P90 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a M at position A117 of SEQ ID NO: 1, a Q at position R119 of SEQ ID NO: 1, and an I at position D123 of SEQ ID NO: 1.

### P90F_D123I

In some embodiments, a KOD polymerase variant comprises: a F at a position corresponding to P90 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and an I at position D123 of SEQ ID NO: 1. In some embodiments, a KOD polymerase variant comprises: a F at position P90 of SEQ ID NO: 1, and an I at position D123 of SEQ ID NO: 1.

### P90F_F116P

In some embodiments, a KOD polymerase variant comprises: a F at a position corresponding to P90 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a P at position F116 of SEQ ID NO: 1. In some embodiments, a KOD polymerase variant comprises: a F at position P90 of SEQ ID NO: 1, and a P at position F116 of SEQ ID NO: 1.

### P90F_F116P_A117M

In some embodiments, a KOD polymerase variant comprises: a F at a position corresponding to P90 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a P at position F116 of SEQ ID NO: 1, and a M at position A117 of SEQ ID NO: 1.

### P90F_F116P_A117M_D123I

In some embodiments, a KOD polymerase variant comprises: a F at a position corresponding to P90 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a P at position F116 of SEQ ID NO: 1, a M at position A117 of SEQ ID NO: 1, and an I at position D123 of SEQ ID NO: 1.

### P90F_F116P_A117M_R119Q

In some embodiments, a KOD polymerase variant comprises: a F at a position corresponding to P90 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a F at position P90 of SEQ ID NO: 1, a P at position F116 of SEQ ID NO: 1, a M at position A117 of SEQ ID NO: 1, and a Q at position R119 of SEQ ID NO: 1.

### P90F_F116P_A117M_R119Q_D123I

In some embodiments, a KOD polymerase variant comprises: a F at a position corresponding to P90 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P90F_F116P_D123I

In some embodiments, a KOD polymerase variant comprises: a F at a position corresponding to P90 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, and an I at position D123 of SEQ ID NO: 1. In some embodiments, a KOD polymerase variant comprises: a F at position P90 of SEQ ID NO: 1, a P at position F116 of SEQ ID NO: 1, and an I at position D123 of SEQ ID NO: 1.

### P90F_F116P_R119Q_D123I

In some embodiments, a KOD polymerase variant comprises: a F at a position corresponding to P90 of SEQ ID NO: 1, a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a P at position F116 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a P at position F116 of SEQ ID NO: 1, a Q at position R119 of SEQ ID NO: 1, and an I at position D123 of SEQ ID NO: 1.

### P90F_P94D

In some embodiments, a KOD polymerase variant comprises: a F at a position corresponding to P90 of SEQ ID NO: 1 and a D at a position corresponding to P94 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a D at position P94 of SEQ ID NO: 1. In some embodiments, a KOD polymerase variant comprises: a F at position P90 of SEQ ID NO: 1, and a D at position P94 of SEQ ID NO: 1.

### P90F_P94D_A117M

In some embodiments, a KOD polymerase variant comprises: a F at a position corresponding to P90 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, and a M at position A117 of SEQ ID NO: 1. In some embodiments, a KOD polymerase variant comprises: a F at position P90 of SEQ ID NO: 1, a D at position P94 of SEQ ID NO: 1, and a M at position A117 of SEQ ID NO: 1.

### P90F_P94D_A117M_D123I

In some embodiments, a KOD polymerase variant comprises: a F at a position corresponding to P90 of SEQ ID NO: 1, a D at a position corresponding to P94 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a D at position P94 of SEQ ID NO: 1, a M at position A117 of SEQ ID NO: 1, and an I at position D123 of SEQ ID NO: 1.

### P90F_P94D_F116P

In some embodiments, a KOD polymerase variant comprises: a F at a position corresponding to P90 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, and a P at position F116 of SEQ ID NO: 1. In some embodiments, a KOD polymerase variant comprises: a F at position P90 of SEQ ID NO: 1, a D at position P94 of SEQ ID NO: 1, and a P at position F116 of SEQ ID NO: 1.

### P90F_P94D_F116P_A117M

In some embodiments, a KOD polymerase variant comprises: a F at a position corresponding to P90 of SEQ ID NO: 1, a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a D at position P94 of SEQ ID NO: 1, a P at position F116 of SEQ ID NO: 1, and a M at position A117 of SEQ ID NO: 1.

### P90F_P94D_F116P_A117M_D123I

In some embodiments, a KOD polymerase variant comprises: a F at a position corresponding to P90 of SEQ ID NO: 1, a D at a position corresponding to P94 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a D at position P94 of SEQ ID NO: 1, a P at position F116 of SEQ ID NO: 1, a M at position A117 of SEQ ID NO: 1, and an I at position D123 of SEQ ID NO: 1.

### P90F_P94D_R97F_F116P_A117M_D123I

In some embodiments, a KOD polymerase variant comprises: a F at a position corresponding to P90 of SEQ ID NO: 1, a D at a position corresponding to P94 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a D at position P94 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a D at position P94 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1.

### P90F_R97F

In some embodiments, a KOD polymerase variant comprises: a F at a position corresponding to P90 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a F at position R97 of SEQ ID NO: 1. In some embodiments, a KOD polymerase variant comprises: a F at position P90 of SEQ ID NO: 1, and a F at position R97 of SEQ ID NO: 1.

### P90F_R97F_A117M

In some embodiments, a KOD polymerase variant comprises: a F at a position corresponding to P90 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, and a M at position A117 of SEQ ID NO: 1. In some embodiments, a KOD polymerase variant comprises: a F at position P90 of SEQ ID NO: 1, a F at position R97 of SEQ ID NO: 1, and a M at position A117 of SEQ ID NO: 1.

### P90F_R97F_A117M_D123I

In some embodiments, a KOD polymerase variant comprises: a F at a position corresponding to P90 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, a M at position A117 of SEQ ID NO: 1, and an I at position D123 of SEQ ID NO: 1.

### P90F_R97F_A117M_R119Q_D123I

In some embodiments, a KOD polymerase variant comprises: a F at a position corresponding to P90 of SEQ ID NO: 1, a F at a position corresponding to R97 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, a M at position A117 of SEQ ID NO: 1, a Q at position R119 of SEQ ID NO: 1, and an I at position D123 of SEQ ID NO: 1.

### P90F_R97F_D123I

In some embodiments, a KOD polymerase variant comprises: a F at a position corresponding to P90 of SEQ ID NO: 1, a F at a position corresponding to R97 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, and an I at position D123 of SEQ ID NO: 1.

### P90F_R97F_F116P

In some embodiments, a KOD polymerase variant comprises: a F at a position corresponding to P90 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, and a P at position F116 of SEQ ID NO: 1. In some embodiments, a KOD polymerase variant comprises: a F at position P90 of SEQ ID NO: 1, a F at position R97 of SEQ ID NO: 1, and a P at position F116 of SEQ ID NO: 1.

### P90F_R97F_F116P_A117M

In some embodiments, a KOD polymerase variant comprises: a F at a position corresponding to P90 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a F at position R97 of SEQ ID NO: 1, a P at position F116 of SEQ ID NO: 1, and a M at position A117 of SEQ ID NO: 1.

### P90F_R97F_F116P_A117M_D123I

In some embodiments, a KOD polymerase variant comprises: a F at a position corresponding to P90 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1.

### P90F_R97F_F116P_A117M_R119Q_D123I

In some embodiments, a KOD polymerase variant comprises: a F at a position corresponding to P90 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P90F_R97F_F116P_D123I

In some embodiments, a KOD polymerase variant comprises: a F at a position corresponding to P90 of SEQ ID NO: 1, a F at a position corresponding to R97 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a F at position R97 of SEQ ID NO: 1, a P at position F116 of SEQ ID NO: 1, and an I at position D123 of SEQ ID NO: 1. In some embodiments, a KOD polymerase variant comprises: a F at position P90 of SEQ ID NO: 1, a F at position R97 of SEQ ID NO: 1, a P at position F116 of SEQ ID NO: 1, and an I at position D123 of SEQ ID NO: 1.

### P90F_R119Q_D123I

In some embodiments, a KOD polymerase variant comprises: a F at a position corresponding to P90 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a Q at position R119 of SEQ ID NO: 1, and an I at position D123 of SEQ ID NO: 1.

### P90F_V93K

In some embodiments, a KOD polymerase variant comprises: a F at a position corresponding to P90 of SEQ ID NO: 1 and a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, and a K at position V93 of SEQ ID NO: 1. In some embodiments, a KOD polymerase variant comprises: a F at position P90 of SEQ ID NO: 1, and a K at position V93 of SEQ ID NO: 1.

### P90F_V93K_A117M

In some embodiments, a KOD polymerase variant comprises: a F at a position corresponding to P90 of SEQ ID NO: 1, a K at a position corresponding to V93 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, and a M at position A117 of SEQ ID NO: 1.

### P90F_V93K_F116P

In some embodiments, a KOD polymerase variant comprises: a F at a position corresponding to P90 of SEQ ID NO: 1, a K at a position corresponding to V93 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a P at position F116 of SEQ ID NO: 1. In some embodiments, a KOD polymerase variant comprises: a F at position P90 of SEQ ID NO: 1, a K at position V93 of SEQ ID NO: 1, and a P at position F116 of SEQ ID NO: 1.

### P90F_V93K_F116P_A117M

In some embodiments, a KOD polymerase variant comprises: a F at a position corresponding to P90 of SEQ ID NO: 1, a K at a position corresponding to V93 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a P at position F116 of SEQ ID NO: 1, and a M at position A117 of SEQ ID NO: 1.

### P90F_V93K_F116P_A117M_D123I

In some embodiments, a KOD polymerase variant comprises: a F at a position corresponding to P90 of SEQ ID NO: 1, a K at a position corresponding to V93 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a P at position F116 of SEQ ID NO: 1, a M at position A117 of SEQ ID NO: 1, and an I at position D123 of SEQ ID NO: 1.

### P90F_V93K_F116P_A117M_R119Q

In some embodiments, a KOD polymerase variant comprises: a F at a position corresponding to P90 of SEQ ID NO: 1, a K at a position corresponding to V93 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a F at position P90 of SEQ ID NO: 1, a K at position V93 of SEQ ID NO: 1, a P at position F116 of SEQ ID NO: 1, a M at position A117 of SEQ ID NO: 1, and a Q at position R119 of SEQ ID NO: 1.

### P90F_V93K_F116P_A117M_R119Q_D123I

In some embodiments, a KOD polymerase variant comprises: a F at a position corresponding to P90 of SEQ ID NO: 1, a K at a position corresponding to V93 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P90F_V93K_P94D

In some embodiments, a KOD polymerase variant comprises: a F at a position corresponding to P90 of SEQ ID NO: 1, a K at a position corresponding to V93 of SEQ ID NO: 1, and a D at a position corresponding to P94 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a D at position P94 of SEQ ID NO: 1. In some embodiments, a KOD polymerase variant comprises: a F at position P90 of SEQ ID NO: 1, a K at position V93 of SEQ ID NO: 1, and a D at position P94 of SEQ ID NO: 1.

### P90F_V93K_P94D_A117M

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, and a M at position A117 of SEQ ID NO: 1.

### P90F_V93K_P94D_F116P

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, and a P at position F116 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, and a P at position F116 of SEQ ID NO: 1.

### P90F_V93K_P94D_F116P_A117M

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a P at position F116 of SEQ ID NO: 1, and a M at position A117 of SEQ ID NO: 1.

### P90F_V93K_P94D_F116P_A117M_R119Q

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a P at position F116 of SEQ ID NO: 1, a M at position A117 of SEQ ID NO: 1, and a Q at position R119 of SEQ ID NO: 1.

### P90F_V93K_P94D_F116P_A117M_R119Q_D123I

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P90F_V93K_P94D_R97F_A117M

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a F at position R97 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a F at position R97 of SEQ ID NO: 1, and a M at position A117 of SEQ ID NO: 1.

### P90F_V93K_P94D_R97F_F116P

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a F at position R97 of SEQ ID NO: 1, and a P at position F116 of SEQ ID NO: 1.

### P90F_V93K_P94D_R97F_F116P_A117M

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a F at position R97 of SEQ ID NO: 1, a P at position F116 of SEQ ID NO: 1, and a M at position A117 of SEQ ID NO: 1.

### P90F_V93K_P94D_R97F_F116P_A117M_R119Q_D123I

In some embodiments, a KOD polymerase variant comprises: a F at a position corresponding to P90 of SEQ ID NO: 1, a K at a position corresponding to V93 of SEQ ID NO: 1, a D at a position corresponding to P94 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a D at position P94 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a D at position P94 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P90F_V93K_R97F

In some embodiments, a KOD polymerase variant comprises: a F at a position corresponding to P90 of SEQ ID NO: 1, a K at a position corresponding to V93 of SEQ ID NO: 1, and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, and a F at position R97 of SEQ ID NO: 1. In some embodiments, a KOD polymerase variant comprises: a F at position P90 of SEQ ID NO: 1, a K at position V93 of SEQ ID NO: 1, and a F at position R97 of SEQ ID NO: 1.

### P90F_V93K_R97F_A117M

In some embodiments, a KOD polymerase variant comprises: a F at a position corresponding to P90 of SEQ ID NO: 1, a K at a position corresponding to V93 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a F at position R97 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a F at position R97 of SEQ ID NO: 1, and a M at position A117 of SEQ ID NO: 1.

### P90F_V93K_R97F_F116P_A117M

In some embodiments, a KOD polymerase variant comprises: a F at a position corresponding to P90 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a F at position P90 of SEQ ID NO: 1, a K at position V93 of SEQ ID NO: 1, a F at position R97 of SEQ ID NO: 1, a P at position F116 of SEQ ID NO: 1, and a M at position A117 of SEQ ID NO: 1.

### P90F_V93K_R97F_F116P_A117M_D123I

In some embodiments, a KOD polymerase variant comprises: a F at a position corresponding to P90 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, and an I at position D123 of SEQ ID NO: 1.

### P90F_V93K_R97F_F116P_A117M_R119Q

In some embodiments, a KOD polymerase variant comprises: a F at a position corresponding to P90 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, and a Q at position R119 of SEQ ID NO: 1. In some embodiments, a KOD polymerase variant comprises: a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, and a Q at position R119 of SEQ ID NO: 1.

### P90F_V93K_R97F_F116P_A117M_R119Q_D123I

In some embodiments, a KOD polymerase variant comprises: a F at a position corresponding to P90 of SEQ ID NO: 1, a K at a position corresponding to V93 of SEQ ID NO: 1, a F at a position corresponding to R97 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position P90 of SEQ ID NO: 1, a K at position V93 of SEQ ID NO: 1, a F at position R97 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### P94D_A117M

In some embodiments, a KOD polymerase variant comprises: a D at a position corresponding to P94 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position P94 of SEQ ID NO: 1, and a M at position A117 of SEQ ID NO: 1. In some embodiments, a KOD polymerase variant comprises: a D at position P94 of SEQ ID NO: 1, and a M at position A117 of SEQ ID NO: 1.

### P94D_A117M_D123I

In some embodiments, a KOD polymerase variant comprises: a D at a position corresponding to P94 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position P94 of SEQ ID NO: 1, a M at position A117 of SEQ ID NO: 1, and an I at position D123 of SEQ ID NO: 1. In some embodiments, a KOD polymerase variant comprises: a D at position P94 of SEQ ID NO: 1, a M at position A117 of SEQ ID NO: 1, and an I at position D123 of SEQ ID NO: 1.

### P94D_F116P

In some embodiments, a KOD polymerase variant comprises: a D at a position corresponding to P94 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position P94 of SEQ ID NO: 1, and a P at position F116 of SEQ ID NO: 1. In some embodiments, a KOD polymerase variant comprises: a D at position P94 of SEQ ID NO: 1, and a P at position F116 of SEQ ID NO: 1.

### P94D_F116P_A117M

In some embodiments, a KOD polymerase variant comprises: a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position P94 of SEQ ID NO: 1, a P at position F116 of SEQ ID NO: 1, and a M at position A117 of SEQ ID NO: 1. In some embodiments, a KOD polymerase variant comprises: a D at position P94 of SEQ ID NO: 1, a P at position F116 of SEQ ID NO: 1, and a M at position A117 of SEQ ID NO: 1.

### P94D_R97F_A117M_D123I

In some embodiments, a KOD polymerase variant comprises: a D at a position corresponding to P94 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position P94 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a D at position P94 of SEQ ID NO: 1, a F at position R97 of SEQ ID NO: 1, a M at position A117 of SEQ ID NO: 1, and an I at position D123 of SEQ ID NO: 1.

### R97F

In some embodiments, a KOD polymerase variant comprises: a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1. In some embodiments, a KOD polymerase variant comprises: a F at position R97 of SEQ ID NO: 1.

### R97F_A117M

In some embodiments, a KOD polymerase variant comprises: a F at a position corresponding to R97 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, and a M at position A117 of SEQ ID NO: 1. In some embodiments, a KOD polymerase variant comprises: a F at position R97 of SEQ ID NO: 1, and a M at position A117 of SEQ ID NO: 1.

### R97F_A117M_D123I

In some embodiments, a KOD polymerase variant comprises: a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a M at position A117 of SEQ ID NO: 1, and an I at position D123 of SEQ ID NO: 1. In some embodiments, a KOD polymerase variant comprises: a F at position R97 of SEQ ID NO: 1, a M at position A117 of SEQ ID NO: 1, and an I at position D123 of SEQ ID NO: 1.

### R97F_A117M_R119Q

In some embodiments, a KOD polymerase variant comprises: a F at a position corresponding to R97 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a M at position A117 of SEQ ID NO: 1, and a Q at position R119 of SEQ ID NO: 1. In some embodiments, a KOD polymerase variant comprises: a F at position R97 of SEQ ID NO: 1, a M at position A117 of SEQ ID NO: 1, and a Q at position R119 of SEQ ID NO: 1.

### R97F_A117M_R119Q_D123I

In some embodiments, a KOD polymerase variant comprises: a F at a position corresponding to R97 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a F at position R97 of SEQ ID NO: 1, a M at position A117 of SEQ ID NO: 1, a Q at position R119 of SEQ ID NO: 1, and an I at position D123 of SEQ ID NO: 1.

### R97F_D123I

In some embodiments, a KOD polymerase variant comprises: a F at a position corresponding to R97 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, and an I at position D123 of SEQ ID NO: 1. In some embodiments, a KOD polymerase variant comprises: a F at position R97 of SEQ ID NO: 1, and an I at position D123 of SEQ ID NO: 1.

### R97F_F116P

In some embodiments, a KOD polymerase variant comprises: a F at a position corresponding to R97 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, and a P at position F116 of SEQ ID NO: 1. In some embodiments, a KOD polymerase variant comprises: a F at position R97 of SEQ ID NO: 1, and a P at position F116 of SEQ ID NO: 1.

### R97F_F116P_A117M

In some embodiments, a KOD polymerase variant comprises: a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a P at position F116 of SEQ ID NO: 1, and a M at position A117 of SEQ ID NO: 1. In some embodiments, a KOD polymerase variant comprises: a F at position R97 of SEQ ID NO: 1, a P at position F116 of SEQ ID NO: 1, and a M at position A117 of SEQ ID NO: 1.

### R97F_F116P_A117M_D123I

In some embodiments, a KOD polymerase variant comprises: a F at a position corresponding to R97 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a F at position R97 of SEQ ID NO: 1, a P at position F116 of SEQ ID NO: 1, a M at position A117 of SEQ ID NO: 1, and an I at position D123 of SEQ ID NO: 1.

### R97F_F116P_D123I

In some embodiments, a KOD polymerase variant comprises: a F at a position corresponding to R97 of SEQ ID NO: 1, a P at a position corresponding to F116 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a F at position R97 of SEQ ID NO: 1, a P at position F116 of SEQ ID NO: 1, and an I at position D123 of SEQ ID NO: 1. In some embodiments, a KOD polymerase variant comprises: a F at position R97 of SEQ ID NO: 1, a P at position F116 of SEQ ID NO: 1, and an I at position D123 of SEQ ID NO: 1.

### R119Q

In some embodiments, a KOD polymerase variant comprises: a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position R119 of SEQ ID NO: 1. In some embodiments, a KOD polymerase variant comprises: a Q at position R119 of SEQ ID NO: 1.

### R119Q_D123I

In some embodiments, a KOD polymerase variant comprises: a Q at a position corresponding to R119 of SEQ ID NO: 1 and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a Q at position R119 of SEQ ID NO: 1, and an I at position D123 of SEQ ID NO: 1. In some embodiments, a KOD polymerase variant comprises: a Q at position R119 of SEQ ID NO: 1, and an I at position D123 of SEQ ID NO: 1.

### V93K

In some embodiments, a KOD polymerase variant comprises: a K at a position corresponding to V93 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1. In some embodiments, a KOD polymerase variant comprises: a K at position V93 of SEQ ID NO: 1.

### V93K_A117M

In some embodiments, a KOD polymerase variant comprises: a K at a position corresponding to V93 of SEQ ID NO: 1 and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a M at position A117 of SEQ ID NO: 1. In some embodiments, a KOD polymerase variant comprises: a K at position V93 of SEQ ID NO: 1, and a M at position A117 of SEQ ID NO: 1.

### V93K_A117M_D123I

In some embodiments, a KOD polymerase variant comprises: a K at a position corresponding to V93 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a M at position A117 of SEQ ID NO: 1, and an I at position D123 of SEQ ID NO: 1. In some embodiments, a KOD polymerase variant comprises: a K at position V93 of SEQ ID NO: 1, a M at position A117 of SEQ ID NO: 1, and an I at position D123 of SEQ ID NO: 1.

### V93K_A117M_R119Q

In some embodiments, a KOD polymerase variant comprises: a K at a position corresponding to V93 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a M at position A117 of SEQ ID NO: 1, and a Q at position R119 of SEQ ID NO: 1. In some embodiments, a KOD polymerase variant comprises: a K at position V93 of SEQ ID NO: 1, a M at position A117 of SEQ ID NO: 1, and a Q at position R119 of SEQ ID NO: 1.

### V93K_A117M_R119Q_D123I

In some embodiments, a KOD polymerase variant comprises: a K at a position corresponding to V93 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a M at position A117 of SEQ ID NO: 1, a Q at position R119 of SEQ ID NO: 1, and an I at position D123 of SEQ ID NO: 1.

### V93K_F116P

In some embodiments, a KOD polymerase variant comprises: a K at a position corresponding to V93 of SEQ ID NO: 1 and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a P at position F116 of SEQ ID NO: 1. In some embodiments, a KOD polymerase variant comprises: a K at position V93 of SEQ ID NO: 1, and a P at position F116 of SEQ ID NO: 1.

### V93K_F116P_A117M

In some embodiments, a KOD polymerase variant comprises: a K at a position corresponding to V93 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a P at position F116 of SEQ ID NO: 1, and a M at position A117 of SEQ ID NO: 1. In some embodiments, a KOD polymerase variant comprises: a K at position V93 of SEQ ID NO: 1, a P at position F116 of SEQ ID NO: 1, and a M at position A117 of SEQ ID NO: 1.

### V93K_F116P_A117M_R119Q_D123I

In some embodiments, a KOD polymerase variant comprises: a K at a position corresponding to V93 of SEQ ID NO: 1, a P at a position corresponding to F116 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a P at position F116 of SEQ ID NO: 1, a M at position A117 of SEQ ID NO: 1, a Q at position R119 of SEQ ID NO: 1, and an I at position D123 of SEQ ID NO: 1.

### V93K_F116P_R119Q_D123I

In some embodiments, a KOD polymerase variant comprises: a K at a position corresponding to V93 of SEQ ID NO: 1, a P at a position corresponding to F116 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a P at position F116 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a P at position F116 of SEQ ID NO: 1, a Q at position R119 of SEQ ID NO: 1, and an I at position D123 of SEQ ID NO: 1.

### V93K_P94D

In some embodiments, a KOD polymerase variant comprises: a K at a position corresponding to V93 of SEQ ID NO: 1 and a D at a position corresponding to P94 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a D at position P94 of SEQ ID NO: 1. In some embodiments, a KOD polymerase variant comprises: a K at position V93 of SEQ ID NO: 1, and a D at position P94 of SEQ ID NO: 1.

### V93K_P94D_A117M

In some embodiments, a KOD polymerase variant comprises: a K at a position corresponding to V93 of SEQ ID NO: 1, a D at a position corresponding to P94 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, and a M at position A117 of SEQ ID NO: 1. In some embodiments, a KOD polymerase variant comprises: a K at position V93 of SEQ ID NO: 1, a D at position P94 of SEQ ID NO: 1, and a M at position A117 of SEQ ID NO: 1.

### V93K_P94D_A117M_R119Q

In some embodiments, a KOD polymerase variant comprises: a K at a position corresponding to V93 of SEQ ID NO: 1, a D at a position corresponding to P94 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, a M at position A117 of SEQ ID NO: 1, and a Q at position R119 of SEQ ID NO: 1. In some embodiments, a KOD polymerase variant comprises: a K at position V93 of SEQ ID NO: 1, a D at position P94 of SEQ ID NO: 1, a M at position A117 of SEQ ID NO: 1, and a Q at position R119 of SEQ ID NO: 1.

### V93K_P94D_A117M_R119Q_D123I

In some embodiments, a KOD polymerase variant comprises: a K at a position corresponding to V93 of SEQ ID NO: 1, a D at a position corresponding to P94 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a D at position P94 of SEQ ID NO: 1, a M at position A117 of SEQ ID NO: 1, a Q at position R119 of SEQ ID NO: 1, and an I at position D123 of SEQ ID NO: 1.

### V93K_P94D_F116P

In some embodiments, a KOD polymerase variant comprises: a K at a position corresponding to V93 of SEQ ID NO: 1, a D at a position corresponding to P94 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, and a P at position F116 of SEQ ID NO: 1. In some embodiments, a KOD polymerase variant comprises: a K at position V93 of SEQ ID NO: 1, a D at position P94 of SEQ ID NO: 1, and a P at position F116 of SEQ ID NO: 1.

### V93K_P94D_F116P_A117M

In some embodiments, a KOD polymerase variant comprises: a K at a position corresponding to V93 of SEQ ID NO: 1, a D at a position corresponding to P94 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, a P at position F116 of SEQ ID NO: 1, and a M at position A117 of SEQ ID NO: 1. In some embodiments, a KOD polymerase variant comprises: a K at position V93 of SEQ ID NO: 1, a D at position P94 of SEQ ID NO: 1, a P at position F116 of SEQ ID NO: 1, and a M at position A117 of SEQ ID NO: 1.

### V93K_P94D_R97F_A117M

In some embodiments, a KOD polymerase variant comprises: a K at a position corresponding to V93 of SEQ ID NO: 1, a D at a position corresponding to P94 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a D at position P94 of SEQ ID NO: 1, a F at position R97 of SEQ ID NO: 1, and a M at position A117 of SEQ ID NO: 1. In some embodiments, a KOD polymerase variant comprises: a K at position V93 of SEQ ID NO: 1, a D at position P94 of SEQ ID NO: 1, a F at position R97 of SEQ ID NO: 1, and a M at position A117 of SEQ ID NO: 1.

### V93K_R97F

In some embodiments, a KOD polymerase variant comprises: a K at a position corresponding to V93 of SEQ ID NO: 1 and a F at a position corresponding to R97 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a F at position R97 of SEQ ID NO: 1. In some embodiments, a KOD polymerase variant comprises: a K at position V93 of SEQ ID NO: 1, and a F at position R97 of SEQ ID NO: 1.

### V93K_R97F_A117M

In some embodiments, a KOD polymerase variant comprises: a K at a position corresponding to V93 of SEQ ID NO: 1, a F at a position corresponding to R97 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, and a M at position A117 of SEQ ID NO: 1. In some embodiments, a KOD polymerase variant comprises: a K at position V93 of SEQ ID NO: 1, a F at position R97 of SEQ ID NO: 1, and a M at position A117 of SEQ ID NO: 1.

### V93K_R97F_A117M_D123I

In some embodiments, a KOD polymerase variant comprises: a K at a position corresponding to V93 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a K at position V93 of SEQ ID NO: 1, a F at position R97 of SEQ ID NO: 1, a M at position A117 of SEQ ID NO: 1, and an I at position D123 of SEQ ID NO: 1.

### V93K_R97F_A117M_R119Q

In some embodiments, a KOD polymerase variant comprises: a K at a position corresponding to V93 of SEQ ID NO: 1, a F at a position corresponding to R97 of SEQ ID NO: 1, a M at a position corresponding to A117 of SEQ ID NO: 1, and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, a M at position A117 of SEQ ID NO: 1, and a Q at position R119 of SEQ ID NO: 1. In some embodiments, a KOD polymerase variant comprises: a K at position V93 of SEQ ID NO: 1, a F at position R97 of SEQ ID NO: 1, a M at position A117 of SEQ ID NO: 1, and a Q at position R119 of SEQ ID NO: 1.

### V93K_R97F_A117M_R119Q_D123I

In some embodiments, a KOD polymerase variant comprises: a K at a position corresponding to V93 of SEQ ID NO: 1, a F at a position corresponding to R97 of SEQ ID NO: 1, a M at a position corresponding to A117 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, a M at position A117 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a F at position R97 of SEQ ID NO: 1, a M at position A117 of SEQ ID NO: 1, a Q at position R119 of SEQ ID NO: 1, and an I at position D123 of SEQ ID NO: 1.

### V93K_R97F_F116P

In some embodiments, a KOD polymerase variant comprises: a K at a position corresponding to V93 of SEQ ID NO: 1, a F at a position corresponding to R97 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, and a P at position F116 of SEQ ID NO: 1. In some embodiments, a KOD polymerase variant comprises: a K at position V93 of SEQ ID NO: 1, a F at position R97 of SEQ ID NO: 1, and a P at position F116 of SEQ ID NO: 1.

### V93K_R97F_F116P_A117M

In some embodiments, a KOD polymerase variant comprises: a K at a position corresponding to V93 of SEQ ID NO: 1, a F at a position corresponding to R97 of SEQ ID NO: 1, a P at a position corresponding to F116 of SEQ ID NO: 1, and a M at a position corresponding to A117 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a F at position R97 of SEQ ID NO: 1, a P at position F116 of SEQ ID NO: 1, and a M at position A117 of SEQ ID NO: 1. In some embodiments, a KOD polymerase variant comprises: a K at position V93 of SEQ ID NO: 1, a F at position R97 of SEQ ID NO: 1, a P at position F116 of SEQ ID NO: 1, and a M at position A117 of SEQ ID NO: 1.

### V93K_R119Q

In some embodiments, a KOD polymerase variant comprises: a K at a position corresponding to V93 of SEQ ID NO: 1 and a Q at a position corresponding to R119 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, and a Q at position R119 of SEQ ID NO: 1. In some embodiments, a KOD polymerase variant comprises: a K at position V93 of SEQ ID NO: 1, and a Q at position R119 of SEQ ID NO: 1.

### V93K_R119Q_D123I

In some embodiments, a KOD polymerase variant comprises: a K at a position corresponding to V93 of SEQ ID NO: 1, a Q at a position corresponding to R119 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a K at position V93 of SEQ ID NO: 1, a Q at position R119 of SEQ ID NO: 1, and an I at position D123 of SEQ ID NO: 1. In some embodiments, a KOD polymerase variant comprises: a K at position V93 of SEQ ID NO: 1, a Q at position R119 of SEQ ID NO: 1, and an I at position D123 of SEQ ID NO: 1.

### Y7D_P90F_F116P

In some embodiments, a KOD polymerase variant comprises: a D at a position corresponding to Y7 of SEQ ID NO: 1, a F at a position corresponding to P90 of SEQ ID NO: 1, and a P at a position corresponding to F116 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y7 of SEQ ID NO: 1, a F at position P90 of SEQ ID NO: 1, and a P at position F116 of SEQ ID NO: 1. In some embodiments, a KOD polymerase variant comprises: a D at position Y7 of SEQ ID NO: 1, a F at position P90 of SEQ ID NO: 1, and a P at position F116 of SEQ ID NO: 1.

### Y37D_A117M_D123I

In some embodiments, a KOD polymerase variant comprises: a D at a position corresponding to Y37 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y37 of SEQ ID NO: 1, a M at position A117 of SEQ ID NO: 1, and an I at position D123 of SEQ ID NO: 1. In some embodiments, a KOD polymerase variant comprises: a D at position Y37 of SEQ ID NO: 1, a M at position A117 of SEQ ID NO: 1, and an I at position D123 of SEQ ID NO: 1.

### Y37D_F116P_A117M_D123I

In some embodiments, a KOD polymerase variant comprises: a D at a position corresponding to Y37 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y37 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a D at position Y37 of SEQ ID NO: 1, a P at position F116 of SEQ ID NO: 1, a M at position A117 of SEQ ID NO: 1, and an I at position D123 of SEQ ID NO: 1.

### Y37D_P90F_F116P_A117M_D123I

In some embodiments, a KOD polymerase variant comprises: a D at a position corresponding to Y37 of SEQ ID NO: 1, a F at a position corresponding to P90 of SEQ ID NO: 1, a P at a position corresponding to F116 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y37 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1. In some embodiments, a KOD polymerase variant comprises: a D at position Y37 of SEQ ID NO: 1, a F at position P90 of SEQ ID NO: 1, a P at position F116 of SEQ ID NO: 1, a M at position A117 of SEQ ID NO: 1, and an I at position D123 of SEQ ID NO: 1.

### Y37D_R97F_A117M_D123I

In some embodiments, a KOD polymerase variant comprises: a D at a position corresponding to Y37 of SEQ ID NO: 1, a F at a position corresponding to R97 of SEQ ID NO: 1, a M at a position corresponding to A117 of SEQ ID NO: 1, and an I at a position corresponding to D123 in SEQ ID NO: 1. In some embodiments, a KOD polymerase variant comprises an amino acid sequence having at least 90% (e.g., at least 95%, at least 98%, or at least 99%) identity with SEQ ID NO: 1; and a D at position Y37 of SEQ ID NO: 1, a F at position R97 of SEQ ID NO: 1, a M at position A117 of SEQ ID NO: 1, and an I at position D123 of SEQ ID NO: 1. In some embodiments, a KOD polymerase variant comprises: a D at position Y37 of SEQ ID NO: 1, a F at position R97 of SEQ ID NO: 1, a M at position A117 of SEQ ID NO: 1, and an I at position D123 of SEQ ID NO: 1.

### Methods of Use

Aspects of this disclosure are drawn to methods of amplifying one or more polynucleotides using KOD polymerase variants with improved uracil template tolerance, inosine template tolerance, and/or uracil incorporation tolerance. In some embodiments, this disclosure provides a method of amplifying one or more DNA polynucleotides comprising inosine, an inosine derivate, uracil, and/or a uracil derivative, the method comprising contacting the one or more DNA polynucleotides with a KOD polymerase variant provided herein.

In some embodiments, the KOD polymerase variants provided herein are useful for amplifying polynucleotides comprising inosine or inosine derivatives. In some embodiments, the disclosure provides a method comprising amplifying a polynucleotide comprising inosine or inosine derivatives using a KOD polymerase variant with inosine template tolerance. In some embodiments, the disclosure provides a method comprising amplifying a polynucleotide containing inosine or inosine derivatives using a KOD polymerase variant with inosine template tolerance listed in Table 1, Table 4, or Table 5. In some embodiments, the disclosure provides a method comprising amplifying a polynucleotide containing inosine, wherein the polynucleotide containing inosine is generated by extending a primer bound to a first DNA strand, wherein the primer comprises inosine or an inosine derivative using a KOD polymerase variant with inosine template tolerance. In some embodiments, the disclosure provides a method comprising amplifying a polynucleotide containing inosine, wherein the polynucleotide containing inosine is generated by amplifying a template DNA strand via incorporation of dITPs using a KOD polymerase variant with inosine incorporation tolerance.

In some embodiments, the KOD polymerase variants provided herein are useful for amplifying polynucleotides comprising uracil or uracil derivatives. In some embodiments, the disclosure provides a method comprising amplifying a polynucleotide comprising uracil or uracil derivatives using a KOD polymerase variant with uracil template tolerance. In some embodiments, the disclosure provides a method comprising amplifying a polynucleotide containing uracil or uracil derivatives using a KOD polymerase variant with uracil template tolerance listed in Table 1, Table 4, or Table 5. In some embodiments, the disclosure provides a method comprising amplifying a polynucleotide containing uracil using a KOD polymerase variant with uracil template tolerance and/or uracil incorporation tolerance. In some embodiments, the polynucleotide containing uracil is generated by extending a primer bound to a first DNA strand, wherein the primer comprises uracil or a uracil derivative using a KOD polymerase variant with uracil primer tolerance. In some embodiments, the disclosure provides a method comprising amplifying a polynucleotide containing uracil, wherein the polynucleotide containing uracil is generated by amplifying a template DNA strand via incorporation of dUTPs using a KOD polymerase variant with uracil incorporation tolerance.

In some embodiments, the KOD polymerase variants provided herein are useful for amplifying polynucleotides comprising uracil and inosine (or derivatives thereof). In some embodiments, the disclosure provides a method comprising amplifying a polynucleotide comprising uracil and inosine (or derivatives thereof). In some embodiments, the polynucleotide containing uracil and inosine (or derivatives thereof) is generated by extending a primer bound to a first DNA strand, wherein the primer comprises uracil and inosine (or derivatives thereof), using a KOD polymerase variant with dU/dI primer tolerance. In some embodiments, the disclosure provides a method comprising amplifying a polynucleotide containing uracil and inosine (or derivatives thereof) using a KOD polymerase variant with dU/dI primer tolerance listed in Table 1, Table 4, or Table 5. In some embodiments, the KOD polymerase variants provided herein are useful for amplifying a polynucleotide via incorporation of uracil (dUTP) into the synthesized strand. In some embodiments, the disclosure provides a method comprising amplifying a polynucleotide via incorporation of uracil (dUTP) into the synthesized strand using a KOD polymerase variant with uracil incorporation tolerance. In some embodiments, the disclosure provides a method comprising amplifying a polynucleotide via incorporation of uracil (dUTP) into the synthesized strand using a KOD polymerase variant with uracil incorporation tolerance listed in Table 1, Table 4, or Table 5.

Wild-type archaeal B-family DNA polymerases, including KOD polymerase, can exhibit poor amplification of polynucleotides comprising uracil or inosine or poor incorporation of uracil during amplification because these are not natural bases in DNA and are primarily found in RNA. Additionally, archaeal B-family DNA polymerases possess a binding pocket, which strongly interacts with uracil or inosine and can stall DNA synthesis when uracil or inosine is present in the template DNA.

Accordingly, KOD polymerase variants provided herein that exhibit enhanced uracil template tolerance, inosine template tolerance, and/or uracil incorporation tolerance are useful for amplifying polynucleotides having uracil and/or inosine or are useful for amplification that involves the incorporation of uracil into the synthesized strand. In some embodiments, the disclosure provides a method of amplifying one or more polynucleotides comprising inosine, an inosine derivative, uracil, and/or a uracil derivative. In some embodiments, a method of amplifying one or more polynucleotides comprises contacting the one or more polynucleotides inosine, an inosine derivative, uracil, and/or a uracil derivative with a KOD polymerase variant provided herein to produce one or more synthesized polynucleotides. In some embodiments, the method comprises amplifying polynucleotides via incorporation of uracil (dUTP) into the synthesized strand.

In some embodiments, a method of amplifying one or more polynucleotides further comprises sequencing the one or more amplified polynucleotides. In some embodiments, sequencing comprises next-generation sequencing (NGS). In some embodiments, sequencing comprises shotgun sequencing. In some embodiments, sequencing comprises long-read sequencing. In some embodiments, sequencing comprises short-read sequencing. In some embodiments, sequencing comprises nanopore sequencing.

Amplifying polynucleotides using a KOD polymerase variant provided herein can be performed using polymerase chain reaction (PCR). PCR amplifies DNA through repeated cycles of denaturing DNA, annealing primers complementary to the DNA, and extending DNA, which requires a wide range of temperatures (e.g., approximately 50 °C to 95 °C), to create large amounts of DNA from even small amounts of starting material. PCR reactions combine target DNA to be amplified, primers complementary to the target DNA or a portion thereof, nucleotides (dNTPs), buffer solutions, ions, and a DNA polymerase (e.g., a KOD polymerase variant or a Pfu polymerase variant provided herein) to amplify the target DNA. Alternatively, amplifying may be linear amplification using a KOD polymerase variant described herein.

A range of pH values of PCR reaction conditions can be used in the methods described herein. In some embodiments, PCR reaction conditions comprise a pH of 7.0 to 10.0. In some embodiments, PCR reaction conditions comprise a pH of 7.5 to 10.0. In some embodiments, PCR reaction conditions comprise a pH of 8.0 to 10.0. In some embodiments, PCR reaction conditions comprise a pH of 8.5 to 10.0. In some embodiments, PCR reaction conditions comprise a pH of 9.0 to 10.0. In some embodiments, PCR reaction conditions comprise a pH of 9.5 to 10.0. In some embodiments, PCR reaction conditions comprise a pH of 8.2 to 9.2. In some embodiments, PCR reaction conditions comprise a pH of 8.4 to 9.0. In some embodiments, PCR reaction conditions comprise a pH of 8.6 to 8.8. In some embodiments, PCR reaction conditions comprise a pH of 8.8 to 9.8. In some embodiments, PCR reaction conditions comprise a pH of 9.0 to 9.6. In some embodiments, PCR reaction conditions comprise a pH of 9.2 to 9.4.

In some embodiments, PCR reaction conditions comprise an extension temperature of 65 °C to 80 °C. In some embodiments, PCR reaction conditions comprise an extension temperature of 70 °C to 80 °C. In some embodiments, PCR reaction conditions comprise an extension temperature of 75 °C to 80 °C. In some embodiments, PCR reaction conditions comprise an extension temperature of 65 °C to 78 °C. In some embodiments, PCR reaction conditions comprise an extension temperature of 65 °C to 76 °C. In some embodiments, PCR reaction conditions comprise an extension temperature of 65 °C to 74 °C. In some embodiments, PCR reaction conditions comprise an extension temperature of 65 °C to 72 °C. In some embodiments, PCR reaction conditions comprise an extension temperature of 65 °C to 70 °C. In some embodiments, PCR reaction conditions comprise an extension temperature of 68 °C to 74 °C.

PCR protocols are known in the art (see, e.g., Zhu et al. PCR past, present and future. Biotechniques. 2020, 10.2144/btn-2020-0057).

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

### EXAMPLES

### Example 1. Single Amino Acid Substitutions in KOD Polymerase Result in Altered dU/dI Template Tolerance, dU Template Tolerance, and/or dUTP Incorporation Tolerance

Encounter of uracil or inosine bases by polymerases can stall polymerization, as some commonly-used polymerases (e.g., family B DNA polymerases from archaea, such as Thermococcus kodakarensis, or KOD, polymerase) possess a binding pocket with a strong affinity for uracil or inosine. In this Example, variants of KOD polymerase with single amino acid substitutions were identified that exhibited varying dU/dI template tolerance, dU template tolerance, and/or dUTP incorporation tolerance.

Thousands of single substitution KOD polymerase variants (with substitutions at more than one hundred different amino acid positions) were produced and tested for polymerization activity with modified primers containing uracil and inosine bases, primers containing uracil bases, or with dUTP using a compartmentalized self-replication (CSR) assay. A plasmid library containing the mutant sequences was transformed into competent E. coli cells and induced to express the variant polymerases. Cells containing polymerase variants and their encoding plasmid were encapsulated in water-in-oil emulsions. During encapsulation, additional components necessary for PCR were added, including either the primers or dNTPs applying the screening pressure (i.e., dU/dI containing primers, dU containing primers, or dUTP), such that the resulting droplets became miniature reaction vessels for DNA amplification to occur.

Droplets contained an appropriate PCR buffer. The PCR primers targeted up- and downstream of the polymerase variant genes on the plasmid and in some conditions included inosine and/or uracil bases. Under conditions with dI and dU containing primers, exponential amplification is only possible with KOD polymerase variants that are able to amplify a template strand with dI and dU located in the primer region (i.e., dU/dI primer tolerant variants). Under conditions with dU containing primers, exponential amplification is only possible with KOD polymerase variants that are able to amplify a template strand with dU located in the primer region (i.e., dU primer tolerant variants). dU/dI primer tolerant or dU primer tolerant variants can read through inosine or uracil bases in the template strand, incorporated from primers that were extended in the preceding cycle of PCR. In another condition, dUTP was present during the amplification reaction. Under conditions with dUTP present, exponential amplification is only possible with KOD polymerase variants that can successfully incorporate dUTP into the synthesized strand and then subsequently use those newly synthesized, uracil-containing strands as templates during subsequent cycles of the PCR (i.e., dUTP incorporation tolerant variants).

Relative activity was determined by a single round of compartmentalized self-replication (CSR) followed by NGS library preparation and sequencing of the enriched pool. Variant frequencies, a direct product of self-replication efficiencies, were counted by NGS sequencing and compared to the frequency of the wildtype enzyme.

The estimation of variant fitness scores and aggregation across samples follows the methods described in the Enrich2 model (Rubin et al., 2017). The Enrich2 methods are slightly adapted to allow the estimation of wild-type performance in fully saturated single site variant libraries.

dU primer data were combined from 12 samples in which the screening pressure ranged from 7-26 dU bases in the primers used during CSR. Pairwise Spearman's correlation coefficients were measured between independent samples. The correlation between samples in conditions with varying uracil content was similar to that between samples within the same conditions. Thus, the conditions with varying uracil content were collapses into a single group describing dU primer tolerant variants. This allowed for a unified estimate of variant fitness in the presence of dU containing primers, increasing statistical power by increasing sample size.

dU_and_dI primer data were combined from 6 samples in which the screening pressure ranged from combinations of 2-14 dU and dI bases in the primers used during CSR. Pairwise Spearman's correlation coefficients were measured between independent samples. The correlation between samples in conditions with varying uracil/inosine content was similar to that between samples within the same conditions. Thus, the conditions with varying uracil/inosine content were collapses into a single group describing dU/dI primer tolerant variants. This allowed for a unified estimate of variant fitness in the presence of dU containing primers, increasing statistical power by increasing sample size.

dUTP data were combined from 8 samples in which the screening pressures included 425 uM dUTP in addition to 425 uM dTTP, 212 uM of each dUTP and dTTP, 425 uM dUTP and no dTTP, or 425 uM dUTP, no dTTP and primers containing 26 dU bases. Pairwise Spearman's correlation coefficients were measured between independent samples. The correlation between samples in conditions with varying dUTP concentrations was similar to that between samples within the same conditions. Thus, the conditions with varying dUTP concentrations were collapses into a single group describing dUTP incorporation tolerant variants. This allowed for a unified estimate of variant fitness in the presence of dU containing primers, increasing statistical power by increasing sample size.

The p-value represents the probability that the variant is better than wild type due to pure chance. The p-value is adjusted for multiple testing (Benjamini-Hochberg FDR algorithm). Results were filtered to only include substitutions in the N-terminal domain and the exonuclease domain. Variants were considered to have improved dU/dI primer tolerance, dU primer tolerance, or dUTP incorporation tolerance if they satisfied 2 criteria in the respective screen condition: (a) a fitness_combined score ≥2 and (b) an adjusted p-value ≤0.05. KOD polymerase variants that meet these criteria are statistically significantly improved in at least one of dU/dI primer tolerance, dU primer tolerance, or dUTP incorporation tolerance over wildtype KOD polymerase by four-fold or more.

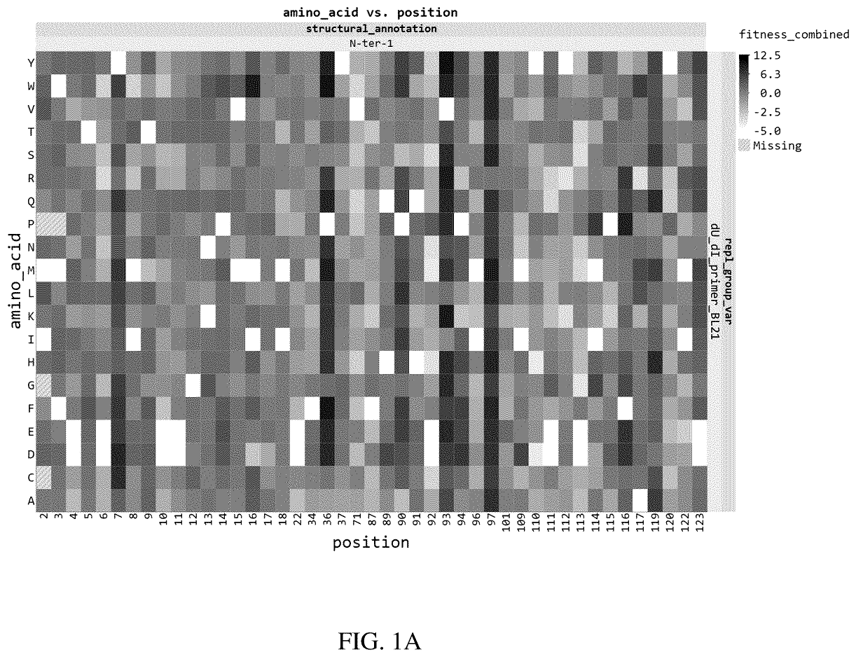

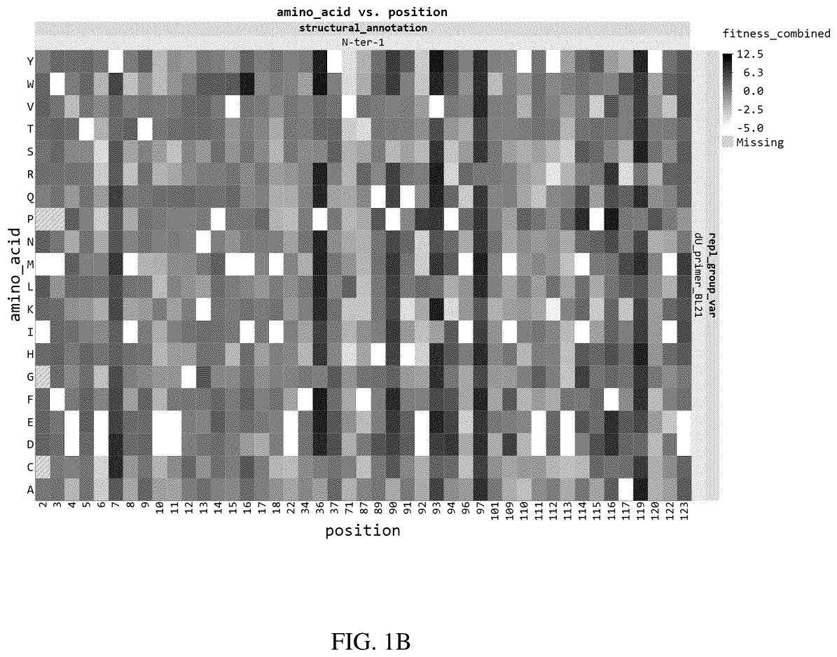

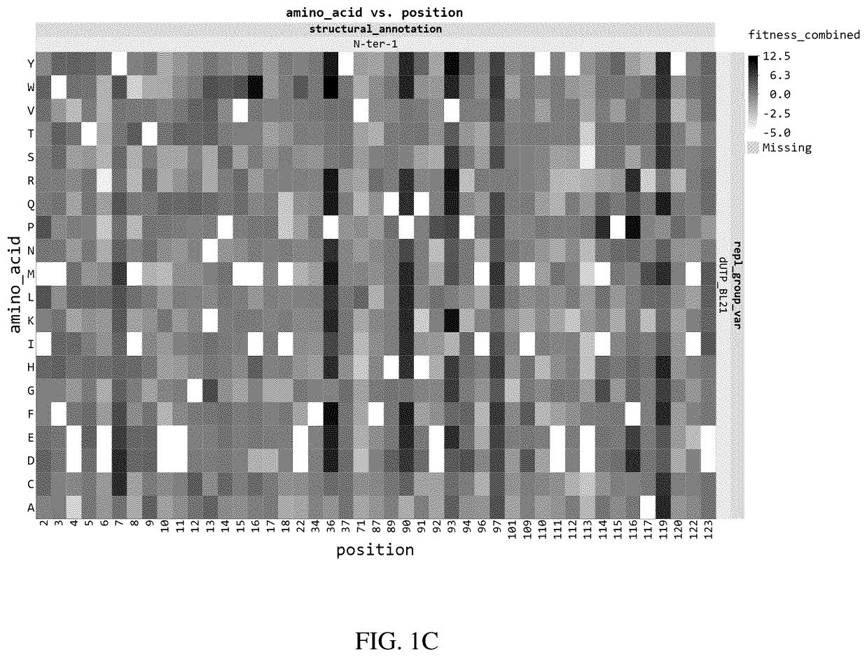

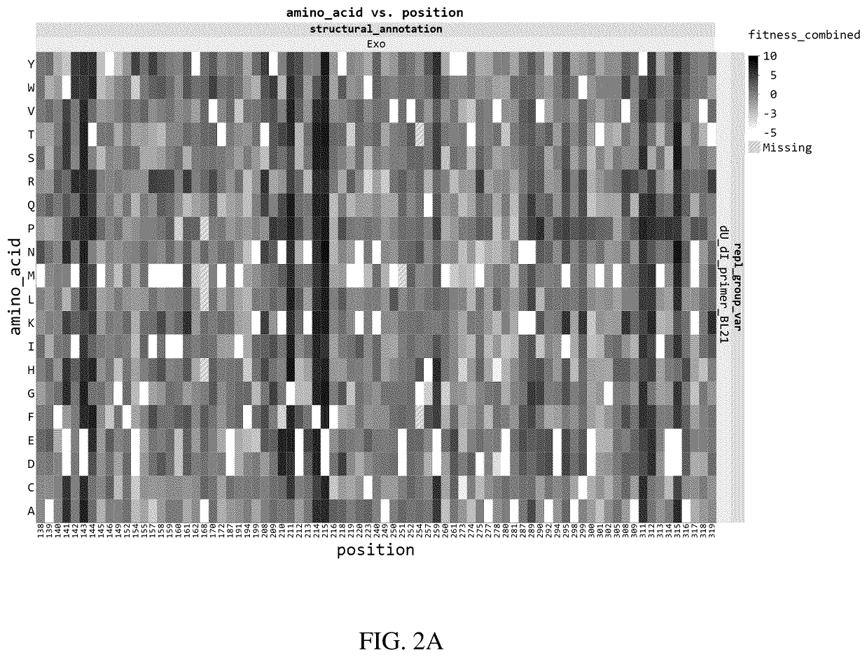

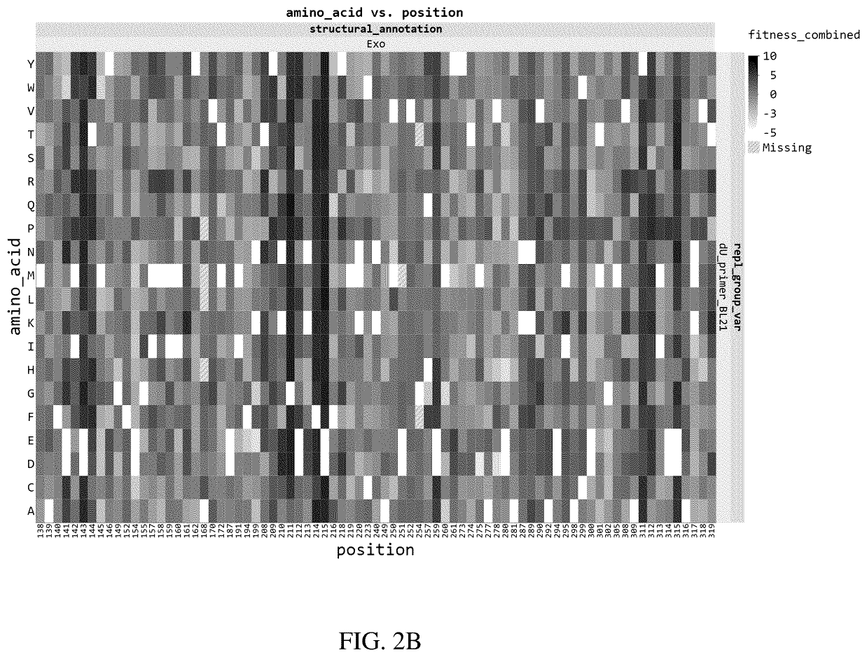

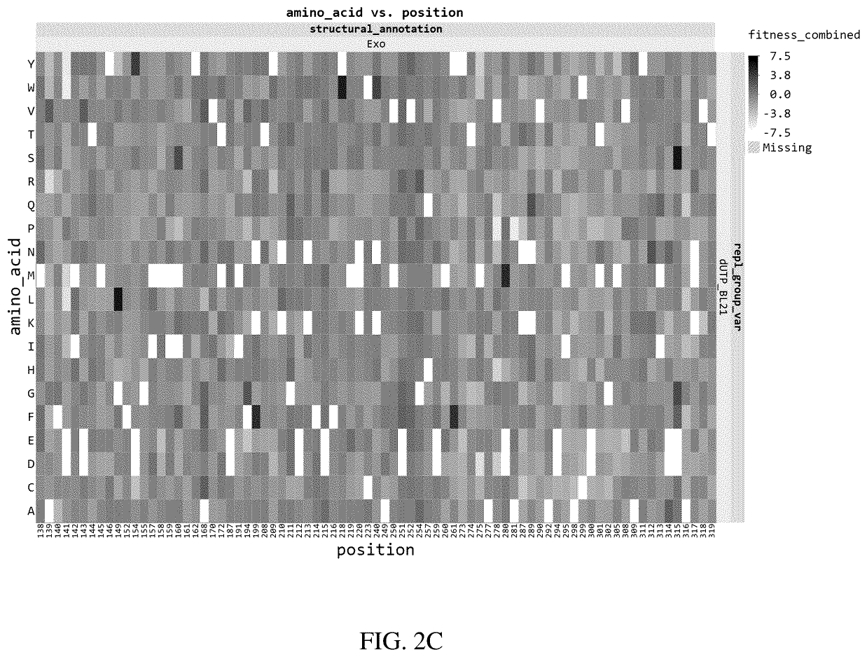

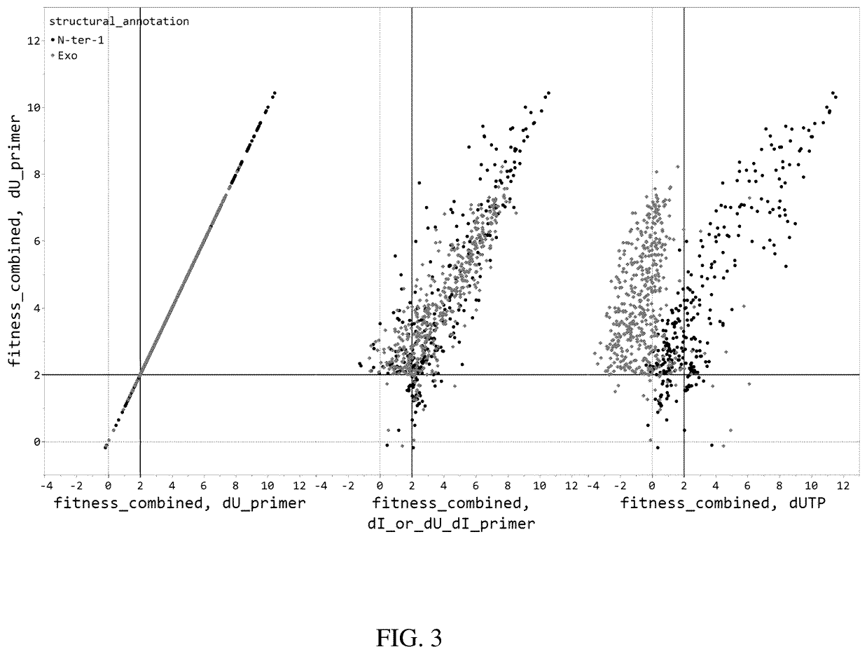

In total, 739 single substitution variants of KOD polymerase (with substitutions across 124 positions) were identified as having significantly improved dU/dI primer tolerance, dU primer tolerance, and/or dUTP incorporation tolerance over the wildtype enzyme in the N-terminal domain and the exonuclease domain. Heatmaps of the fitness_combined score for each amino acid substitution at the identified amino acid positions are shown in FIGS. 1-2. Screen conditions with either dU containing primers or dUTP incorporation identified similar substitutions in the N-terminal domain (FIG. 3). However, in the exonuclease domain, many substitutions that were beneficial for dU primer tolerance (dU containing primers) were not beneficial for dUTP incorporation tolerance (FIG. 3).

A summary of results for each single substitution variant is shown in Table 1.

TABLE 1
Single substitution KOD polymerase variants.

| SEQ | | | | | | | |
|---|---|---|---|---|---|---|---|
| ID | | p-val | | p-val | | p-val | |
| NO | Substitution | dI/dU* | dI/dU** | dU^{†} | dU^{††} | dUTP^{‡} | dUTP^{‡‡} |
| 2 | I2D | 0.000 | 2.683 | 0.000 | 2.707 | 0.000 | 2.789 |
| 3 | I2E | 0.028 | 1.732 | 0.000 | 2.559 | 0.254 | 1.691 |
| 4 | I2F | 1.000 | −0.407 | 0.000 | 2.217 | 1.000 | 0.152 |
| 5 | I2H | 0.022 | 1.916 | 0.000 | 2.140 | 0.000 | 2.422 |
| 6 | I2K | 0.930 | 0.891 | 0.000 | 2.166 | 1.000 | 0.091 |
| 7 | I2L | 0.000 | 3.945 | 0.000 | 3.594 | 0.000 | 4.256 |
| 8 | I2N | 0.042 | 2.235 | 0.000 | 2.943 | 0.169 | 0.928 |
| 9 | I2T | 0.331 | 1.088 | 0.005 | 2.080 | 1.000 | −0.085 |
| 10 | I2V | 0.000 | 3.810 | 0.000 | 2.822 | 0.152 | 1.576 |
| 11 | I2W | 0.000 | 2.780 | 0.000 | 2.532 | 0.000 | 2.777 |
| 12 | L3D | 0.011 | 2.326 | 0.001 | 1.846 | 0.018 | 0.990 |
| 13 | L3H | 0.006 | 2.766 | 0.000 | 2.719 | 0.000 | 3.000 |
| 14 | L3I | 0.000 | 2.565 | 0.000 | 2.230 | 0.000 | 2.492 |
| 15 | L3S | 0.001 | 2.661 | 0.000 | 2.344 | 0.000 | 2.124 |
| 16 | L3T | 0.429 | 1.889 | 0.000 | 2.551 | 0.000 | 3.035 |
| 17 | L3Y | 0.000 | 2.326 | 0.000 | 2.239 | 0.000 | 2.620 |
| 18 | D4I | 0.000 | 2.071 | 0.000 | 1.628 | 0.000 | 2.178 |
| 19 | D4L | 0.000 | 2.496 | 0.000 | 2.192 | 0.000 | 2.352 |
| 20 | D4M | 0.008 | 2.807 | 0.000 | 3.117 | 0.127 | 1.540 |
| 21 | D4P | 0.072 | 1.767 | 0.000 | 3.210 | 1.000 | 0.373 |
| 22 | D4Y | 0.000 | 2.235 | 0.000 | 1.913 | 0.000 | 2.939 |
| 23 | T5D | 0.045 | 1.916 | 0.000 | 2.308 | 0.001 | 1.833 |
| 24 | T5E | 0.002 | 2.297 | 0.000 | 2.988 | 0.000 | 3.011 |
| 25 | T5F | 0.000 | 3.964 | 0.000 | 2.702 | 0.005 | 1.942 |
| 26 | T5G | 0.000 | 2.106 | 0.026 | 1.724 | 1.000 | 0.646 |
| 27 | T5H | 0.000 | 2.111 | 0.000 | 2.502 | 0.000 | 1.930 |
| 28 | T5L | 0.000 | 2.102 | 0.000 | 1.985 | 0.000 | 2.342 |
| 29 | T5W | 0.000 | 1.995 | 0.001 | 2.434 | 0.016 | 1.231 |
| 30 | T5Y | 0.000 | 2.338 | 0.000 | 2.349 | 0.000 | 2.473 |
| 31 | D6L | 0.005 | 1.795 | 0.000 | 1.522 | 0.000 | 2.112 |
| 32 | Y7A | 0.003 | 2.912 | 0.000 | 3.556 | 0.000 | 1.518 |
| 33 | Y7C | 0.000 | 7.844 | 0.000 | 8.377 | 0.000 | 8.195 |
| 34 | Y7D | 0.000 | 8.444 | 0.000 | 8.195 | 0.000 | 8.202 |
| 35 | Y7E | 0.000 | 6.270 | 0.000 | 5.991 | 0.000 | 6.275 |
| 36 | Y7F | 0.000 | 6.901 | 0.000 | 5.421 | 0.000 | 5.188 |
| 37 | Y7G | 0.000 | 6.267 | 0.000 | 4.937 | 0.000 | 3.111 |
| 38 | Y7H | 0.000 | 3.120 | 0.000 | 3.118 | 0.000 | 2.626 |
| 39 | Y7I | 0.000 | 3.408 | 0.000 | 3.068 | 0.000 | 2.713 |
| 40 | Y7K | 0.000 | 4.758 | 0.000 | 5.205 | 0.000 | 3.716 |
| 41 | Y7L | 0.000 | 5.790 | 0.000 | 4.896 | 0.000 | 4.485 |
| 42 | Y7M | 0.000 | 6.884 | 0.000 | 6.890 | 0.000 | 6.606 |
| 43 | Y7N | 0.000 | 3.987 | 0.000 | 4.085 | 0.000 | 2.985 |
| 44 | Y7P | 0.000 | 4.386 | 0.000 | 3.670 | 0.000 | 1.627 |
| 45 | Y7Q | 0.000 | 6.735 | 0.000 | 6.187 | 0.000 | 4.189 |
| 46 | Y7R | 0.083 | 1.411 | 0.000 | 3.570 | 0.000 | 1.434 |
| 47 | Y7S | 0.001 | 4.356 | 0.000 | 4.001 | 0.000 | 2.123 |
| 48 | Y7T | 0.097 | 1.845 | 0.000 | 2.123 | 0.000 | 1.105 |
| 49 | Y7V | 0.071 | 2.021 | 0.000 | 2.454 | 0.001 | 1.016 |
| 50 | Y7W | 0.000 | 6.123 | 0.000 | 5.657 | 0.000 | 4.450 |
| 51 | I8D | 0.000 | 2.630 | 0.000 | 2.053 | 0.000 | 2.435 |
| 52 | I8E | 0.000 | 2.104 | 0.000 | 2.150 | 0.000 | 2.488 |
| 53 | I8H | 0.000 | 1.784 | 0.000 | 1.549 | 0.000 | 2.212 |
| 54 | I8T | 0.000 | 3.202 | 0.000 | 2.948 | 0.000 | 3.479 |
| 55 | T9A | 0.000 | 2.773 | 0.000 | 2.554 | 0.000 | 3.085 |
| 56 | T9C | 0.000 | 2.453 | 0.000 | 1.135 | 1.000 | 0.520 |
| 57 | T9D | 0.000 | 3.222 | 0.000 | 2.544 | 0.000 | 2.783 |
| 58 | T9E | 0.000 | 2.328 | 0.000 | 2.038 | 0.000 | 2.549 |
| 59 | T9F | 0.001 | 3.568 | 0.020 | 2.734 | 0.026 | 1.372 |
| 60 | T9H | 0.000 | 2.522 | 0.000 | 1.371 | 0.000 | 1.019 |
| 61 | T9I | 0.031 | 2.176 | 1.000 | 0.483 | 1.000 | −0.257 |
| 62 | T9L | 0.000 | 2.820 | 0.176 | 1.063 | 0.242 | 0.560 |
| 63 | T9N | 0.000 | 2.651 | 0.000 | 1.203 | 0.000 | 1.526 |
| 64 | T9R | 0.000 | 1.700 | 0.000 | 1.531 | 0.000 | 2.057 |
| 65 | T9Y | 0.017 | 2.859 | 0.000 | 2.970 | 0.578 | 0.750 |
| 66 | E10P | 0.000 | 2.014 | 0.186 | 0.645 | 0.001 | 0.917 |
| 67 | E10Q | 0.000 | 1.892 | 0.000 | 1.681 | 0.000 | 2.313 |
| 68 | D11Q | 0.000 | 2.017 | 0.000 | 1.935 | 0.000 | 2.238 |
| 69 | D11T | 0.000 | 2.115 | 0.000 | 1.702 | 0.000 | 2.767 |
| 70 | G12C | 0.000 | 2.752 | 0.000 | 2.391 | 0.000 | 3.575 |
| 71 | G12Q | 0.000 | 2.257 | 0.000 | 2.073 | 0.000 | 2.725 |
| 72 | G12T | 0.000 | 2.251 | 0.000 | 2.099 | 0.000 | 2.514 |
| 73 | G12V | 0.000 | 2.200 | 0.000 | 2.128 | 0.000 | 2.666 |
| 74 | K13G | 0.000 | 4.058 | 0.000 | 3.950 | 0.000 | 5.009 |
| 75 | K13H | 0.000 | 2.228 | 0.000 | 1.280 | 0.080 | 0.925 |
| 76 | K13T | 0.000 | 2.390 | 0.000 | 2.069 | 0.000 | 2.861 |
| 77 | K13V | 0.000 | 2.596 | 0.000 | 2.383 | 0.000 | 3.358 |
| 78 | K13W | 0.000 | 3.444 | 0.000 | 3.479 | 0.000 | 4.443 |
| 79 | P14C | 0.000 | 1.899 | 0.000 | 1.666 | 0.000 | 2.474 |
| 80 | P14D | 0.000 | 3.841 | 0.025 | 1.804 | 0.386 | 0.686 |
| 81 | P14E | 0.000 | 4.597 | 0.000 | 3.430 | 0.003 | 1.395 |
| 82 | P14F | 0.000 | 3.561 | 0.000 | 2.438 | 0.000 | 1.644 |
| 83 | P14H | 0.000 | 2.096 | 0.000 | 1.220 | 1.000 | 0.409 |
| 84 | P14M | 0.000 | 3.377 | 0.001 | 3.089 | 0.000 | 1.322 |
| 85 | P14Q | 0.000 | 2.467 | 0.000 | 1.461 | 0.000 | 0.952 |
| 86 | P14S | 0.000 | 1.809 | 0.000 | 1.642 | 0.000 | 2.339 |
| 87 | P14T | 0.000 | 2.265 | 0.000 | 1.390 | 0.096 | 0.581 |
| 88 | P14W | 0.000 | 4.835 | 0.000 | 3.529 | 0.000 | 3.675 |
| 89 | V15Q | 0.007 | 2.148 | 0.009 | 2.200 | 0.032 | 1.769 |
| 90 | V15W | 0.000 | 3.387 | 0.000 | 3.535 | 0.000 | 4.445 |
| 91 | I16A | 0.000 | 3.347 | 0.032 | 2.002 | 1.000 | 0.276 |
| 92 | I16C | 0.000 | 3.918 | 0.000 | 3.836 | 0.000 | 2.225 |
| 93 | I16F | 0.000 | 3.508 | 0.000 | 2.850 | 0.001 | 1.046 |
| 94 | I16H | 0.071 | 3.321 | 0.069 | 1.810 | 0.001 | 3.231 |
| 95 | I16K | 0.000 | 2.074 | 0.001 | 1.336 | 0.642 | 0.541 |
| 96 | I16S | 0.002 | 2.421 | 0.407 | 1.132 | 0.480 | 0.449 |
| 97 | I16W | 0.000 | 9.445 | 0.000 | 9.848 | 0.000 | 11.119 |
| 98 | I16Y | 0.034 | 3.911 | 0.000 | 2.707 | 0.288 | 0.991 |
| 99 | R17P | 0.000 | 2.410 | 0.000 | 2.194 | 0.000 | 1.029 |
| 100 | I18Y | 0.000 | 2.011 | 0.000 | 1.363 | 0.138 | 0.541 |
| 101 | E22W | 1.000 | 0.442 | 1.000 | −0.113 | 0.002 | 3.748 |
| 102 | F34D | 0.000 | 2.338 | 0.006 | 1.265 | 1.000 | 0.204 |
| 103 | P36C | 0.587 | 1.169 | 1.000 | 0.337 | 0.001 | 2.035 |
| 104 | P36D | 0.000 | 8.442 | 0.000 | 8.699 | 0.000 | 9.247 |
| 105 | P36E | 0.000 | 8.302 | 0.000 | 9.389 | 0.000 | 9.495 |
| 106 | P36F | 0.000 | 10.109 | 0.000 | 9.897 | 0.000 | 11.142 |
| 107 | P36H | 0.000 | 7.255 | 0.000 | 6.997 | 0.000 | 7.966 |
| 108 | P36I | 0.000 | 6.880 | 0.000 | 6.167 | 0.000 | 5.417 |
| 109 | P36K | 0.000 | 7.184 | 0.000 | 8.286 | 0.000 | 8.622 |
| 110 | P36L | 0.000 | 6.976 | 0.000 | 8.879 | 0.000 | 7.488 |
| 111 | P36M | 0.000 | 9.224 | 0.000 | 9.123 | 0.000 | 9.981 |
| 112 | P36N | 0.000 | 7.272 | 0.000 | 8.754 | 0.000 | 7.660 |
| 113 | P36Q | 0.000 | 8.758 | 0.000 | 8.693 | 0.000 | 9.861 |
| 114 | P36R | 0.000 | 6.536 | 0.000 | 9.123 | 0.000 | 10.046 |
| 115 | P36T | 0.000 | 2.921 | 0.001 | 2.441 | 0.000 | 1.033 |
| 116 | P36V | 0.687 | 1.251 | 0.015 | 2.115 | 0.000 | 1.421 |
| 117 | P36W | 0.000 | 10.347 | 0.000 | 10.309 | 0.000 | 11.514 |
| 118 | P36Y | 0.000 | 8.957 | 0.000 | 9.314 | 0.000 | 9.827 |
| 119 | Y37A | 1.000 | −1.277 | 0.005 | 2.333 | 0.074 | 1.291 |
| 120 | Y37D | 0.033 | 1.350 | 0.000 | 4.564 | 0.000 | 1.946 |
| 121 | Y37E | 0.118 | 0.840 | 0.000 | 3.855 | 0.000 | 1.285 |
| 122 | Y37F | 0.005 | 2.154 | 0.027 | 1.805 | 0.000 | 1.135 |
| 123 | Y37G | 0.037 | 2.397 | 0.000 | 3.605 | 0.000 | 1.715 |
| 124 | Y37S | 1.000 | −0.427 | 0.001 | 2.904 | 0.000 | 1.050 |
| 125 | Y37T | 0.007 | 1.740 | 0.000 | 2.563 | 0.003 | 1.110 |
| 126 | V71L | 0.003 | 2.033 | 0.003 | 2.750 | 0.045 | 2.562 |
| 127 | F87R | 0.747 | 1.033 | 0.000 | 2.444 | 0.000 | 1.689 |
| 128 | H89D | 0.000 | 5.353 | 0.000 | 4.238 | 0.000 | 2.972 |
| 129 | H89P | 0.000 | 2.838 | 0.000 | 3.977 | 0.000 | 1.505 |
| 130 | H89W | 0.141 | 1.881 | 0.000 | 2.609 | 0.088 | 0.926 |
| 131 | P90C | 0.004 | 3.402 | 0.000 | 3.819 | 0.000 | 2.866 |
| 132 | P90D | 0.000 | 4.586 | 0.000 | 5.798 | 0.000 | 7.009 |
| 133 | P90E | 0.000 | 6.116 | 0.000 | 6.935 | 0.000 | 8.317 |
| 134 | P90F | 0.000 | 6.336 | 0.000 | 8.288 | 0.000 | 9.179 |
| 135 | P90G | 0.201 | 1.346 | 0.002 | 2.746 | 0.000 | 1.108 |
| 136 | P90H | 0.001 | 4.433 | 0.000 | 5.879 | 0.000 | 7.647 |
| 137 | P90I | 0.000 | 6.392 | 0.000 | 6.753 | 0.000 | 8.001 |
| 138 | P90K | 0.006 | 4.325 | 0.000 | 5.244 | 0.000 | 8.394 |
| 139 | P90L | 0.000 | 7.279 | 0.000 | 6.109 | 0.000 | 8.615 |
| 140 | P90M | 0.000 | 5.185 | 0.000 | 6.780 | 0.000 | 8.356 |
| 141 | P90N | 0.002 | 4.389 | 0.000 | 5.270 | 0.000 | 5.199 |
| 142 | P90Q | 0.003 | 5.543 | 0.000 | 5.619 | 0.000 | 7.702 |
| 143 | P90R | 0.000 | 4.838 | 0.000 | 6.027 | 0.000 | 7.388 |
| 144 | P90W | 0.000 | 6.755 | 0.000 | 6.521 | 0.000 | 8.981 |
| 145 | P90Y | 0.000 | 5.905 | 0.000 | 6.587 | 0.000 | 8.502 |
| 146 | Q91D | 0.053 | 2.232 | 0.000 | 3.295 | 0.000 | 1.980 |
| 147 | Q91E | 0.121 | 2.632 | 0.007 | 2.864 | 1.000 | 0.847 |
| 148 | Q91N | 0.682 | 0.577 | 0.000 | 2.298 | 1.000 | 0.365 |
| 149 | Q91Y | 0.002 | 3.059 | 0.000 | 3.067 | 0.000 | 2.468 |
| 150 | D92P | 0.000 | 3.461 | 0.000 | 6.419 | 0.000 | 4.519 |
| 151 | V93A | 0.000 | 5.218 | 0.000 | 5.831 | 0.000 | 3.173 |
| 152 | V93C | 0.000 | 3.627 | 0.000 | 2.794 | 0.000 | 1.853 |
| 153 | V93D | 0.000 | 6.528 | 0.000 | 6.199 | 0.000 | 3.581 |
| 154 | V93E | 0.000 | 8.189 | 0.000 | 8.811 | 0.000 | 8.004 |
| 155 | V93F | 0.000 | 6.022 | 0.000 | 5.080 | 0.000 | 2.265 |
| 156 | V93G | 0.000 | 8.190 | 0.000 | 7.786 | 0.000 | 6.513 |
| 157 | V93H | 0.000 | 8.139 | 0.000 | 8.101 | 0.000 | 6.474 |
| 158 | V93K | 0.000 | 9.657 | 0.000 | 9.544 | 0.000 | 10.736 |
| 159 | V93M | 0.000 | 7.216 | 0.000 | 7.071 | 0.000 | 4.338 |
| 160 | V93N | 0.000 | 4.487 | 0.000 | 4.396 | 0.000 | 3.728 |
| 161 | V93P | 0.000 | 7.822 | 0.000 | 7.025 | 0.000 | 5.494 |
| 162 | V93Q | 0.000 | 8.453 | 0.000 | 8.762 | 0.000 | 9.340 |
| 163 | V93R | 0.000 | 9.606 | 0.000 | 9.519 | 0.000 | 10.203 |
| 164 | V93S | 0.000 | 8.129 | 0.000 | 7.263 | 0.000 | 6.857 |
| 165 | V93T | 0.000 | 4.735 | 0.000 | 4.875 | 0.000 | 3.470 |
| 166 | V93W | 0.000 | 8.998 | 0.000 | 8.997 | 0.000 | 8.675 |
| 167 | V93Y | 0.000 | 10.551 | 0.000 | 10.434 | 0.000 | 11.342 |
| 168 | P94C | 0.002 | 2.029 | 0.004 | 2.251 | 1.000 | 0.314 |
| 169 | P94D | 0.000 | 6.871 | 0.000 | 7.025 | 0.000 | 4.595 |
| 170 | P94E | 0.000 | 5.533 | 0.000 | 5.729 | 0.833 | 1.720 |
| 171 | P94F | 0.000 | 6.017 | 0.000 | 4.236 | 0.000 | 2.571 |
| 172 | P94G | 0.028 | 2.330 | 0.159 | 1.073 | 0.999 | 0.588 |
| 173 | P94H | 0.000 | 5.252 | 0.000 | 3.918 | 0.000 | 1.665 |
| 174 | P94I | 0.078 | 2.959 | 0.000 | 3.479 | 0.006 | 2.266 |
| 175 | P94M | 0.000 | 3.396 | 0.000 | 3.324 | 1.000 | 0.698 |
| 176 | P94N | 0.001 | 3.639 | 0.011 | 2.041 | 0.015 | 0.745 |
| 177 | P94Q | 0.033 | 2.159 | 0.002 | 2.285 | 1.000 | −0.372 |
| 178 | P94W | 0.000 | 4.604 | 0.000 | 3.246 | 0.001 | 1.438 |
| 179 | P94Y | 0.000 | 4.924 | 0.000 | 3.936 | 0.001 | 3.647 |
| 180 | I96P | 1.000 | −0.050 | 0.006 | 2.044 | 0.015 | 0.748 |
| 181 | I96R | 0.326 | 1.052 | 0.000 | 2.067 | 0.540 | 0.510 |
| 182 | R97A | 0.000 | 7.614 | 0.000 | 7.738 | 0.000 | 4.431 |
| 183 | R97C | 0.000 | 6.249 | 0.000 | 6.125 | 0.000 | 3.984 |
| 184 | R97D | 0.000 | 7.517 | 0.000 | 7.811 | 0.000 | 5.335 |
| 185 | R97E | 0.000 | 7.685 | 0.000 | 7.078 | 0.000 | 5.777 |
| 186 | R97F | 0.000 | 8.518 | 0.000 | 8.745 | 0.000 | 6.867 |
| 187 | R97G | 0.000 | 6.206 | 0.000 | 5.307 | 0.000 | 3.740 |
| 188 | R97H | 0.000 | 8.754 | 0.000 | 8.323 | 0.000 | 6.380 |
| 189 | R97I | 0.000 | 6.712 | 0.000 | 6.171 | 0.000 | 4.716 |
| 190 | R97K | 0.000 | 5.033 | 0.000 | 4.065 | 0.000 | 1.775 |
| 191 | R97L | 0.000 | 7.950 | 0.000 | 8.088 | 0.000 | 5.480 |
| 192 | R97M | 0.000 | 9.137 | 0.000 | 9.359 | 0.000 | 7.140 |
| 193 | R97N | 0.000 | 6.636 | 0.000 | 6.903 | 0.000 | 4.020 |
| 194 | R97P | 0.000 | 6.332 | 0.000 | 7.092 | 0.000 | 5.600 |
| 195 | R97Q | 0.000 | 7.394 | 0.000 | 6.819 | 0.000 | 5.344 |
| 196 | R97S | 0.000 | 8.375 | 0.000 | 7.006 | 0.000 | 4.931 |
| 197 | R97T | 0.000 | 6.334 | 0.000 | 6.014 | 0.000 | 3.075 |
| 198 | R97V | 0.000 | 8.410 | 0.000 | 8.362 | 0.000 | 5.488 |
| 199 | R97W | 0.000 | 8.350 | 0.000 | 7.963 | 0.000 | 6.916 |
| 200 | R97Y | 0.000 | 8.191 | 0.000 | 8.122 | 0.000 | 5.922 |
| 201 | R101E | 0.000 | 2.222 | 0.359 | 0.875 | 1.000 | 0.357 |
| 202 | R101Q | 0.000 | 2.079 | 1.000 | −0.185 | 1.000 | 0.360 |
| 203 | I109D | 0.000 | 5.613 | 0.000 | 5.985 | 0.000 | 4.021 |
| 204 | I109F | 0.695 | 1.095 | 0.003 | 3.684 | 0.058 | 3.582 |
| 205 | Y110P | 0.176 | 1.588 | 0.000 | 2.746 | 1.000 | 0.691 |
| 206 | E111G | 0.000 | 3.285 | 0.000 | 3.075 | 0.001 | 0.905 |
| 207 | Y112D | 0.569 | 0.985 | 0.001 | 2.692 | 1.000 | 0.059 |
| 208 | Y112E | 0.888 | 0.685 | 0.041 | 2.270 | 0.267 | 0.564 |
| 209 | D113P | 0.494 | 1.261 | 0.000 | 2.221 | 0.000 | 1.247 |
| 210 | I114E | 0.158 | 1.202 | 0.000 | 3.921 | 0.001 | 1.253 |
| 211 | I114G | 0.000 | 5.450 | 0.000 | 6.139 | 0.001 | 4.822 |
| 212 | I114H | 0.000 | 2.568 | 0.000 | 3.633 | 0.000 | 2.205 |
| 213 | I114K | 1.000 | −1.197 | 0.000 | 2.264 | 1.000 | −0.117 |
| 214 | I114N | 1.000 | −0.554 | 0.022 | 2.114 | 0.652 | 0.728 |
| 215 | I114P | 0.000 | 6.719 | 0.000 | 8.123 | 0.000 | 7.769 |
| 216 | I114Q | 0.000 | 2.196 | 0.000 | 4.467 | 0.000 | 2.544 |
| 217 | I114S | 1.000 | 0.004 | 0.000 | 3.527 | 0.001 | 0.967 |
| 218 | I114Y | 0.000 | 3.079 | 0.002 | 2.315 | 0.082 | 0.683 |
| 219 | P115C | 0.488 | 0.750 | 0.000 | 2.339 | 1.000 | 0.275 |
| 220 | P115D | 0.047 | 3.260 | 0.000 | 4.445 | 0.000 | 1.624 |
| 221 | P115F | 1.000 | 0.490 | 0.010 | 2.039 | 0.567 | 0.854 |
| 222 | P115H | 0.000 | 1.512 | 0.000 | 2.368 | 0.029 | 0.804 |
| 223 | P115N | 1.000 | 0.670 | 0.001 | 2.762 | 0.000 | 2.083 |
| 224 | P115S | 0.307 | 1.578 | 0.005 | 2.089 | 0.006 | 0.931 |
| 225 | P115T | 0.000 | 2.049 | 0.000 | 1.680 | 0.011 | 1.196 |
| 226 | P115Y | 0.599 | 1.188 | 0.000 | 3.639 | 0.000 | 2.472 |
| 227 | F116A | 0.000 | 2.793 | 0.000 | 3.313 | 1.000 | 0.858 |
| 228 | F116D | 0.000 | 7.227 | 0.000 | 7.626 | 0.000 | 8.110 |
| 229 | F116E | 0.000 | 7.255 | 0.000 | 7.795 | 0.000 | 6.090 |
| 230 | F116G | 0.003 | 5.147 | 0.181 | 2.305 | 0.191 | 2.422 |
| 231 | F116H | 0.000 | 3.633 | 0.000 | 4.541 | 0.008 | 2.380 |
| 232 | F116I | 0.794 | 1.103 | 0.001 | 2.692 | 0.854 | 1.185 |
| 233 | F116K | 0.000 | 2.678 | 0.013 | 2.458 | 0.875 | 0.707 |
| 234 | F116N | 0.437 | 1.317 | 0.000 | 4.990 | 0.000 | 2.457 |
| 235 | F116P | 0.000 | 9.098 | 0.000 | 10.010 | 0.000 | 10.959 |
| 236 | F116Q | 0.000 | 5.591 | 0.000 | 4.658 | 0.000 | 3.165 |
| 237 | F116R | 0.000 | 6.754 | 0.000 | 7.197 | 0.000 | 8.017 |
| 238 | F116V | 0.000 | 2.815 | 0.000 | 3.930 | 0.000 | 1.224 |
| 239 | A117D | 0.004 | 2.370 | 0.000 | 2.464 | 0.000 | 1.087 |
| 240 | A117E | 0.070 | 1.849 | 0.000 | 3.625 | 0.391 | 0.647 |
| 241 | A117L | 0.003 | 4.387 | 0.000 | 3.154 | 0.066 | 1.987 |
| 242 | A117M | 0.000 | 5.019 | 0.000 | 5.100 | 0.000 | 4.711 |
| 243 | A117Q | 0.022 | 2.977 | 0.003 | 2.761 | 0.242 | 0.717 |
| 244 | A117W | 0.000 | 6.181 | 0.000 | 4.226 | 0.000 | 2.022 |
| 245 | R119A | 0.000 | 6.435 | 0.000 | 9.437 | 0.000 | 8.382 |
| 246 | R119C | 0.000 | 4.541 | 0.000 | 6.856 | 0.000 | 7.385 |
| 247 | R119D | 0.661 | 1.066 | 0.000 | 2.250 | 0.001 | 3.450 |
| 248 | R119E | 0.000 | 3.156 | 0.000 | 5.380 | 0.000 | 3.923 |
| 249 | R119F | 0.000 | 2.459 | 0.000 | 7.741 | 0.000 | 8.344 |
| 250 | R119G | 0.070 | 2.909 | 0.000 | 7.003 | 0.000 | 6.243 |
| 251 | R119H | 0.000 | 8.174 | 0.000 | 9.352 | 0.000 | 8.766 |
| 252 | R119I | 0.047 | 3.000 | 0.000 | 6.389 | 0.000 | 4.107 |
| 253 | R119K | 0.017 | 2.236 | 0.000 | 5.991 | 0.000 | 2.168 |
| 254 | R119L | 0.000 | 1.877 | 0.000 | 4.156 | 0.000 | 2.449 |
| 255 | R119M | 0.000 | 6.272 | 0.000 | 7.860 | 0.000 | 7.979 |
| 256 | R119N | 0.912 | 0.951 | 0.000 | 5.554 | 0.000 | 2.977 |
| 257 | R119Q | 0.000 | 7.993 | 0.000 | 7.917 | 0.000 | 9.497 |
| 258 | R119S | 0.000 | 6.523 | 0.000 | 9.146 | 0.000 | 7.418 |
| 259 | R119T | 0.000 | 4.632 | 0.000 | 7.185 | 0.000 | 7.254 |
| 260 | R119V | 0.000 | 4.791 | 0.000 | 6.443 | 0.000 | 5.486 |
| 261 | R119W | 0.000 | 4.438 | 0.000 | 6.947 | 0.000 | 7.575 |
| 262 | R119Y | 0.000 | 5.566 | 0.000 | 8.816 | 0.000 | 8.114 |
| 263 | Y120P | 0.137 | 2.327 | 0.000 | 3.984 | 0.002 | 2.057 |
| 264 | I122K | 1.000 | 0.174 | 0.004 | 2.237 | 1.000 | 0.064 |
| 265 | I122R | 0.000 | 3.447 | 0.000 | 2.721 | 0.540 | 0.384 |
| 266 | I122W | 1.000 | −0.385 | 0.001 | 2.781 | 0.021 | 1.081 |
| 267 | D123A | 0.000 | 1.994 | 0.000 | 3.502 | 0.000 | 1.397 |
| 268 | D123C | 0.317 | 1.582 | 0.002 | 2.647 | 0.003 | 1.420 |
| 269 | D123F | 0.000 | 3.443 | 0.001 | 1.660 | 0.001 | 0.939 |
| 270 | D123H | 0.013 | 2.722 | 0.000 | 3.624 | 1.000 | 1.002 |
| 271 | D123I | 0.062 | 3.278 | 0.000 | 4.924 | 0.000 | 3.923 |
| 272 | D123K | 0.000 | 4.368 | 0.000 | 4.320 | 0.000 | 2.731 |
| 273 | D123L | 0.000 | 4.348 | 0.000 | 5.271 | 0.000 | 3.156 |
| 274 | D123M | 0.000 | 5.518 | 0.000 | 6.523 | 0.000 | 3.656 |
| 275 | D123Q | 0.000 | 4.963 | 0.000 | 2.974 | 0.000 | 1.944 |
| 276 | D123R | 0.000 | 4.667 | 0.001 | 3.109 | 0.000 | 2.142 |
| 277 | D123S | 0.157 | 2.132 | 0.000 | 3.527 | 0.035 | 1.288 |
| 278 | D123T | 0.674 | 1.010 | 0.013 | 2.554 | 0.744 | 0.680 |
| 279 | D123V | 0.000 | 4.297 | 0.000 | 4.596 | 0.000 | 2.240 |
| 280 | D123W | 0.007 | 4.349 | 0.000 | 4.227 | 0.000 | 2.209 |
| 281 | D123Y | 0.000 | 5.787 | 0.000 | 3.704 | 0.000 | 2.607 |
| 282 | L138N | 0.000 | 3.489 | 0.001 | 2.064 | 0.591 | 1.187 |
| 283 | A139E | 0.000 | 2.656 | 0.000 | 2.789 | 1.000 | −1.922 |
| 284 | A139Q | 0.004 | 2.274 | 0.000 | 2.214 | 1.000 | −1.769 |
| 285 | F140R | 0.000 | 2.786 | 0.001 | 2.811 | 1.000 | −2.748 |
| 286 | D141A | 0.000 | 6.002 | 0.000 | 4.610 | 1.000 | −1.062 |
| 287 | D141C | 0.000 | 5.666 | 0.000 | 5.620 | 1.000 | −0.585 |
| 288 | D141G | 0.000 | 6.267 | 0.000 | 5.603 | 1.000 | −1.754 |
| 289 | D141H | 0.000 | 5.595 | 0.000 | 4.457 | 1.000 | −1.104 |
| 290 | D141K | 0.000 | 4.735 | 0.000 | 4.762 | 1.000 | −2.031 |
| 291 | D141N | 0.000 | 6.419 | 0.000 | 6.285 | 1.000 | −1.364 |
| 292 | D141P | 0.000 | 4.093 | 0.000 | 3.609 | 1.000 | 0.346 |
| 293 | D141Q | 0.000 | 3.051 | 0.000 | 2.584 | 1.000 | −2.189 |
| 294 | D141S | 0.000 | 5.110 | 0.000 | 4.600 | 1.000 | −2.294 |
| 295 | D141T | 0.000 | 5.132 | 0.000 | 4.852 | 1.000 | −2.111 |
| 296 | D141V | 0.000 | 4.533 | 0.000 | 3.210 | 1.000 | −0.573 |
| 297 | T142F | 1.000 | 0.337 | 0.000 | 3.330 | 1.000 | −0.007 |
| 298 | I142G | 1.000 | 0.761 | 0.000 | 2.471 | 1.000 | −0.392 |
| 299 | I142H | 0.040 | 2.447 | 0.000 | 3.150 | 0.009 | 0.757 |
| 300 | I142K | 0.000 | 2.384 | 0.000 | 2.464 | 1.000 | 0.340 |
| 301 | I142P | 0.000 | 5.783 | 0.000 | 5.392 | 1.000 | −0.649 |
| 302 | I142R | 0.000 | 6.255 | 0.000 | 5.948 | 1.000 | −1.596 |
| 303 | I142W | 0.000 | 5.420 | 0.000 | 5.095 | 1.000 | −0.293 |
| 304 | I142Y | 0.000 | 5.073 | 0.000 | 5.247 | 0.481 | 0.553 |
| 305 | E143A | 0.000 | 7.199 | 0.000 | 6.621 | 1.000 | −0.505 |
| 306 | E143C | 0.000 | 6.423 | 0.000 | 6.239 | 1.000 | 0.193 |
| 307 | E143F | 0.000 | 6.480 | 0.000 | 5.851 | 1.000 | −0.559 |
| 308 | E143G | 0.000 | 7.126 | 0.000 | 6.938 | 1.000 | 0.374 |
| 309 | E143H | 0.000 | 6.038 | 0.000 | 5.082 | 1.000 | −1.834 |
| 310 | E143I | 0.000 | 5.467 | 0.000 | 5.542 | 1.000 | −0.739 |
| 311 | E143K | 0.000 | 3.970 | 0.000 | 3.522 | 1.000 | −2.035 |
| 312 | E143L | 0.000 | 5.948 | 0.000 | 5.915 | 1.000 | −1.271 |
| 313 | E143M | 0.000 | 6.031 | 0.000 | 6.345 | 1.000 | −1.300 |
| 314 | E143N | 0.000 | 7.094 | 0.000 | 6.799 | 0.401 | 0.445 |
| 315 | E143P | 0.000 | 5.948 | 0.000 | 5.938 | 1.000 | −1.476 |
| 316 | E143Q | 0.000 | 6.986 | 0.000 | 6.696 | 1.000 | −0.182 |
| 317 | E143R | 0.000 | 6.421 | 0.000 | 6.541 | 1.000 | −0.953 |
| 318 | E143S | 0.000 | 6.920 | 0.000 | 5.790 | 1.000 | −0.500 |
| 319 | E143T | 0.000 | 6.964 | 0.000 | 7.050 | 1.000 | −0.283 |
| 320 | E143V | 0.000 | 6.430 | 0.000 | 6.346 | 0.000 | 1.987 |
| 321 | E143W | 0.000 | 5.240 | 0.000 | 4.420 | 1.000 | −1.604 |
| 322 | E143Y | 0.000 | 5.946 | 0.000 | 6.547 | 0.107 | 0.581 |
| 323 | T144A | 0.618 | 1.414 | 0.000 | 3.215 | 1.000 | −1.755 |
| 324 | T144D | 0.000 | 3.552 | 0.000 | 3.400 | 1.000 | −1.454 |
| 325 | T144E | 0.000 | 6.231 | 0.000 | 4.523 | 1.000 | 0.199 |
| 326 | T144F | 0.000 | 7.216 | 0.000 | 6.288 | 1.000 | −0.701 |
| 327 | T144G | 0.725 | 1.081 | 0.000 | 3.930 | 1.000 | −0.520 |
| 328 | T144H | 0.000 | 6.593 | 0.000 | 6.047 | 0.652 | 0.473 |
| 329 | T144K | 0.000 | 5.114 | 0.000 | 5.310 | 1.000 | 0.171 |
| 330 | T144L | 0.000 | 4.171 | 0.000 | 3.900 | 1.000 | −1.703 |
| 331 | T144M | 0.000 | 4.078 | 0.002 | 3.603 | 1.000 | −1.171 |
| 332 | T144P | 0.000 | 6.471 | 0.000 | 6.240 | 1.000 | −0.684 |
| 333 | T144Q | 0.000 | 4.886 | 0.000 | 5.895 | 1.000 | 0.823 |
| 334 | T144R | 0.000 | 5.392 | 0.000 | 4.947 | 1.000 | 0.414 |
| 335 | T144W | 0.000 | 6.409 | 0.000 | 6.401 | 1.000 | −0.300 |
| 336 | T144Y | 0.000 | 5.658 | 0.000 | 5.410 | 0.161 | 0.527 |
| 337 | L145G | 0.712 | 1.091 | 0.000 | 2.695 | 1.000 | −1.874 |
| 338 | Y146D | 0.705 | 1.197 | 0.000 | 3.120 | 1.000 | −2.005 |
| 339 | Y146E | 0.000 | 2.677 | 0.000 | 3.720 | 1.000 | −0.083 |
| 340 | G149L | 1.000 | 0.439 | 0.557 | 1.725 | 0.001 | 6.100 |
| 341 | F152A | 0.108 | 1.135 | 0.000 | 2.023 | 1.000 | −0.029 |
| 342 | F152D | 0.002 | 1.652 | 0.000 | 2.202 | 1.000 | −0.227 |
| 343 | F152E | 0.936 | 0.726 | 0.000 | 2.395 | 1.000 | −0.284 |
| 344 | F152N | 1.000 | −0.158 | 0.000 | 2.730 | 1.000 | −1.563 |
| 345 | F152S | 0.082 | 1.414 | 0.000 | 3.017 | 1.000 | −0.683 |
| 346 | E154P | 1.000 | −0.563 | 0.000 | 2.869 | 1.000 | −2.208 |
| 347 | E154Y | 0.000 | 4.683 | 0.051 | 1.657 | 0.000 | 3.803 |
| 348 | G155I | 0.275 | 2.001 | 0.000 | 4.029 | 1.000 | −0.770 |
| 349 | G155V | 0.056 | 1.984 | 0.000 | 2.804 | 1.000 | −0.373 |
| 350 | I157E | 0.940 | 0.814 | 0.000 | 2.023 | 1.000 | −0.494 |
| 351 | I157F | 0.000 | 3.804 | 0.000 | 3.624 | 1.000 | −0.394 |
| 352 | I157R | 0.000 | 4.807 | 0.000 | 4.601 | 1.000 | −2.020 |
| 353 | I157W | 0.000 | 2.335 | 0.000 | 3.584 | 1.000 | −1.236 |
| 354 | I157Y | 0.000 | 4.802 | 0.000 | 4.490 | 1.000 | −1.239 |
| 355 | L158D | 0.000 | 2.940 | 0.000 | 2.491 | 1.000 | −2.602 |
| 356 | L158H | 0.000 | 3.514 | 0.000 | 3.522 | 1.000 | −0.950 |
| 357 | L158K | 0.000 | 4.031 | 0.000 | 4.465 | 1.000 | −0.002 |
| 358 | L158Q | 0.001 | 2.469 | 0.000 | 3.153 | 1.000 | −1.985 |
| 359 | L158R | 0.000 | 4.389 | 0.000 | 4.981 | 1.000 | −0.992 |
| 360 | L158W | 0.000 | 2.924 | 0.000 | 3.529 | 1.000 | −1.382 |
| 361 | L158Y | 0.180 | 1.637 | 0.000 | 3.349 | 1.000 | −1.523 |
| 362 | M159E | 0.135 | 1.494 | 0.001 | 2.829 | 1.000 | −0.128 |
| 363 | M159G | 0.687 | 1.310 | 0.000 | 3.400 | 1.000 | −0.823 |
| 364 | M159P | 0.002 | 2.508 | 0.000 | 2.101 | 1.000 | −2.394 |
| 365 | M159R | 0.000 | 4.104 | 0.000 | 4.350 | 1.000 | −0.383 |
| 366 | I160F | 0.000 | 2.415 | 0.365 | 1.685 | 0.083 | 1.489 |
| 367 | S161D | 0.000 | 2.636 | 0.000 | 3.504 | 1.000 | −0.340 |
| 368 | S161E | 0.072 | 2.405 | 0.000 | 3.989 | 1.000 | −0.627 |
| 369 | S161F | 0.000 | 3.368 | 0.000 | 2.620 | 1.000 | −1.288 |
| 370 | S161H | 0.000 | 2.977 | 0.000 | 3.881 | 1.000 | −1.810 |
| 371 | S161K | 0.000 | 5.984 | 0.000 | 6.602 | 1.000 | 0.345 |
| 372 | S161L | 0.000 | 3.064 | 0.000 | 3.155 | 1.000 | −0.949 |
| 373 | S161M | 0.000 | 4.060 | 0.000 | 3.806 | 1.000 | −0.781 |
| 374 | S161N | 0.047 | 2.681 | 0.015 | 2.710 | 1.000 | −0.001 |
| 375 | S161P | 0.098 | 2.332 | 0.006 | 3.275 | 1.000 | −1.365 |
| 376 | S161R | 0.000 | 5.022 | 0.000 | 4.323 | 1.000 | −1.171 |
| 377 | S161T | 0.005 | 1.585 | 0.000 | 2.223 | 1.000 | −0.802 |
| 378 | S161W | 0.000 | 3.169 | 0.000 | 3.261 | 1.000 | −1.332 |
| 379 | S161Y | 0.063 | 2.783 | 0.000 | 3.745 | 1.000 | −1.386 |
| 380 | Y162P | 0.031 | 2.181 | 0.000 | 3.921 | 1.000 | 0.074 |
| 381 | A168C | 0.195 | 1.956 | 0.000 | 3.395 | 0.106 | 1.830 |
| 382 | A168E | 0.002 | 1.896 | 0.000 | 2.272 | 0.019 | 1.040 |
| 383 | A168F | 0.004 | 2.701 | 0.001 | 2.183 | 0.009 | 1.838 |
| 384 | A168G | 1.000 | 0.254 | 0.000 | 2.661 | 0.103 | 1.527 |
| 385 | A168N | 0.032 | 1.689 | 0.001 | 2.294 | 0.531 | 1.218 |
| 386 | A168T | 0.138 | 1.747 | 0.001 | 2.201 | 1.000 | −0.060 |
| 387 | V170A | 0.803 | 1.188 | 0.000 | 2.709 | 1.000 | −1.564 |
| 388 | V170G | 0.000 | 2.918 | 0.000 | 4.218 | 1.000 | −0.704 |
| 389 | V170H | 0.007 | 2.335 | 0.000 | 2.990 | 1.000 | −0.923 |
| 390 | V170R | 0.000 | 3.998 | 0.000 | 3.521 | 1.000 | −1.770 |
| 391 | V170S | 1.000 | 0.944 | 0.000 | 3.298 | 1.000 | −1.341 |
| 392 | V170W | 0.000 | 4.767 | 0.000 | 4.515 | 1.000 | −0.014 |
| 393 | V170Y | 0.000 | 2.812 | 0.000 | 3.731 | 1.000 | −1.059 |
| 394 | T172R | 0.900 | 0.732 | 0.006 | 2.232 | 1.000 | −1.514 |
| 395 | E187W | 0.126 | 1.885 | 0.032 | 2.135 | 1.000 | −0.222 |
| 396 | I191F | 0.063 | 1.862 | 0.016 | 2.008 | 1.000 | −0.279 |
| 397 | I191Y | 0.821 | 1.063 | 0.004 | 2.214 | 1.000 | 0.214 |
| 398 | F194G | 0.007 | 1.670 | 0.000 | 2.020 | 0.147 | 2.466 |
| 399 | K199F | 0.559 | 1.644 | 0.007 | 2.681 | 0.000 | 4.649 |
| 400 | T208E | 0.017 | 2.852 | 0.000 | 3.909 | 1.000 | −2.037 |
| 401 | T208F | 0.000 | 3.836 | 0.000 | 4.187 | 1.000 | −0.746 |
| 402 | T208H | 0.000 | 4.066 | 0.000 | 4.749 | 1.000 | 0.040 |
| 403 | T208I | 0.267 | 2.321 | 0.000 | 4.844 | 1.000 | −0.626 |
| 404 | T208K | 0.000 | 5.016 | 0.000 | 4.401 | 1.000 | −0.754 |
| 405 | T208N | 0.000 | 4.472 | 0.000 | 5.735 | 0.411 | 0.783 |
| 406 | T208Q | 0.000 | 4.497 | 0.000 | 4.493 | 1.000 | 0.218 |
| 407 | T208R | 0.000 | 5.698 | 0.000 | 6.069 | 1.000 | 0.259 |
| 408 | T208V | 0.007 | 2.559 | 0.000 | 4.567 | 0.015 | 0.889 |
| 409 | T208W | 0.000 | 3.301 | 0.000 | 4.120 | 1.000 | −1.889 |
| 410 | T208Y | 0.000 | 5.007 | 0.000 | 4.499 | 0.993 | 0.479 |
| 411 | Y209A | 0.397 | 1.486 | 0.000 | 3.887 | 1.000 | −1.652 |
| 412 | Y209C | 0.255 | 1.327 | 0.000 | 3.057 | 1.000 | −1.408 |
| 413 | Y209E | 0.008 | 2.421 | 0.000 | 3.021 | 1.000 | −2.116 |
| 414 | Y209G | 0.000 | 4.763 | 0.000 | 4.397 | 1.000 | −0.865 |
| 415 | Y209H | 0.537 | 1.037 | 0.000 | 2.424 | 1.000 | −0.564 |
| 416 | Y209I | 0.000 | 3.578 | 0.003 | 2.422 | 1.000 | −1.113 |
| 417 | Y209L | 0.020 | 1.941 | 0.001 | 2.844 | 1.000 | −1.276 |
| 418 | Y209M | 0.565 | 1.202 | 0.027 | 2.047 | 1.000 | −0.462 |
| 419 | Y209N | 0.001 | 2.700 | 0.000 | 3.714 | 1.000 | −2.669 |
| 420 | Y209P | 0.000 | 5.258 | 0.000 | 5.950 | 1.000 | −1.806 |
| 421 | Y209S | 0.064 | 2.802 | 0.000 | 3.906 | 1.000 | −1.684 |
| 422 | Y209T | 0.000 | 2.950 | 0.000 | 3.171 | 1.000 | −0.772 |
| 423 | Y209V | 0.000 | 3.413 | 0.000 | 4.793 | 1.000 | −0.325 |
| 424 | Y209W | 0.000 | 5.170 | 0.000 | 5.488 | 1.000 | 0.874 |
| 425 | N210C | 0.125 | 1.713 | 0.000 | 3.058 | 1.000 | −0.939 |
| 426 | N210D | 0.000 | 6.898 | 0.000 | 6.540 | 1.000 | −0.911 |
| 427 | N210E | 0.000 | 6.916 | 0.000 | 5.857 | 1.000 | −0.056 |
| 428 | N210G | 1.000 | 0.596 | 0.022 | 2.374 | 1.000 | −1.318 |
| 429 | N210M | 0.044 | 2.625 | 0.000 | 3.254 | 1.000 | 0.144 |
| 430 | N210P | 0.000 | 4.721 | 0.000 | 4.501 | 1.000 | −0.662 |
| 431 | N210Q | 0.000 | 3.935 | 0.000 | 5.239 | 1.000 | −0.726 |
| 432 | N210S | 0.353 | 1.237 | 0.005 | 2.191 | 1.000 | 0.267 |
| 433 | N210T | 0.000 | 4.278 | 0.000 | 4.596 | 0.197 | 0.515 |
| 434 | G211C | 0.000 | 5.522 | 0.000 | 5.708 | 1.000 | −2.065 |
| 435 | G211D | 0.000 | 5.707 | 0.000 | 7.086 | 0.579 | 0.823 |
| 436 | G211E | 0.000 | 7.072 | 0.000 | 7.232 | 0.257 | 0.831 |
| 437 | G211F | 0.000 | 6.715 | 0.000 | 6.672 | 1.000 | 0.492 |
| 438 | G211H | 0.000 | 7.949 | 0.000 | 7.573 | 0.021 | 1.107 |
| 439 | G211I | 0.000 | 7.237 | 0.000 | 7.264 | 1.000 | 0.361 |
| 440 | G211K | 0.000 | 5.822 | 0.000 | 6.840 | 1.000 | −1.217 |
| 441 | G211L | 0.000 | 7.174 | 0.000 | 6.849 | 1.000 | 0.300 |
| 442 | G211M | 0.000 | 6.332 | 0.000 | 6.974 | 1.000 | −0.021 |
| 443 | G211N | 0.000 | 6.088 | 0.000 | 6.689 | 1.000 | 0.084 |
| 444 | G211P | 0.000 | 7.223 | 0.000 | 7.223 | 0.003 | 1.082 |
| 445 | G211Q | 0.000 | 7.652 | 0.000 | 8.226 | 0.010 | 1.613 |
| 446 | G211R | 0.000 | 4.545 | 0.000 | 5.836 | 1.000 | 0.208 |
| 447 | G211S | 0.000 | 4.035 | 0.000 | 6.811 | 1.000 | −0.219 |
| 448 | G211T | 0.000 | 6.282 | 0.000 | 6.075 | 0.432 | 1.096 |
| 449 | G211V | 0.000 | 6.890 | 0.000 | 6.874 | 1.000 | 0.178 |
| 450 | G211W | 0.000 | 6.163 | 0.000 | 6.174 | 1.000 | −0.117 |
| 451 | G211Y | 0.000 | 4.833 | 0.000 | 5.782 | 1.000 | −0.430 |
| 452 | D212F | 0.000 | 3.057 | 0.000 | 4.968 | 1.000 | 0.191 |
| 453 | D212I | 1.000 | −0.280 | 0.000 | 2.987 | 1.000 | −2.125 |
| 454 | D212K | 0.014 | 1.404 | 0.038 | 2.387 | 1.000 | −1.971 |
| 455 | D212L | 0.000 | 2.655 | 0.000 | 5.295 | 1.000 | 0.266 |
| 456 | D212M | 0.574 | 1.105 | 0.000 | 2.917 | 1.000 | −0.450 |
| 457 | D212P | 0.052 | 3.483 | 0.000 | 5.949 | 0.347 | 0.476 |
| 458 | D212Q | 0.002 | 1.486 | 0.000 | 2.488 | 1.000 | −0.496 |
| 459 | D212R | 0.040 | 1.790 | 0.000 | 3.497 | 1.000 | −1.081 |
| 460 | D212V | 0.000 | 2.344 | 0.000 | 3.429 | 1.000 | 0.217 |
| 461 | D212W | 0.007 | 3.507 | 0.000 | 6.340 | 1.000 | 0.651 |
| 462 | D212Y | 0.000 | 3.753 | 0.000 | 5.971 | 1.000 | 0.152 |
| 463 | N213D | 0.000 | 2.492 | 0.000 | 4.226 | 1.000 | −0.774 |
| 464 | N213E | 0.000 | 4.425 | 0.000 | 4.572 | 1.000 | −0.262 |
| 465 | F214A | 0.000 | 6.182 | 0.000 | 7.080 | 1.000 | 0.194 |
| 466 | F214C | 0.000 | 4.637 | 0.000 | 4.538 | 0.453 | 0.440 |
| 467 | F214D | 0.000 | 6.964 | 0.000 | 6.544 | 0.427 | 0.614 |
| 468 | F214E | 0.000 | 6.978 | 0.000 | 6.513 | 1.000 | 0.179 |
| 469 | F214G | 0.000 | 6.864 | 0.000 | 5.787 | 1.000 | −1.006 |
| 470 | F214H | 0.000 | 6.362 | 0.000 | 6.774 | 1.000 | −0.299 |
| 471 | F214I | 0.000 | 6.810 | 0.000 | 6.265 | 1.000 | −0.405 |
| 472 | F214K | 0.000 | 7.286 | 0.000 | 6.548 | 1.000 | 0.055 |
| 473 | F214L | 0.000 | 6.010 | 0.000 | 5.868 | 1.000 | −0.537 |
| 474 | F214M | 0.000 | 5.479 | 0.000 | 4.637 | 1.000 | 0.162 |
| 475 | F214N | 0.000 | 5.921 | 0.000 | 4.690 | 1.000 | −0.430 |
| 476 | F214P | 0.000 | 6.353 | 0.000 | 5.966 | 1.000 | −0.354 |
| 477 | F214Q | 0.000 | 7.019 | 0.000 | 6.048 | 1.000 | −0.902 |
| 478 | F214R | 0.000 | 6.119 | 0.000 | 6.370 | 1.000 | −0.905 |
| 479 | F214S | 0.000 | 7.277 | 0.000 | 6.740 | 1.000 | 0.212 |
| 480 | F214T | 0.000 | 7.253 | 0.000 | 6.802 | 1.000 | −0.160 |
| 481 | F214V | 0.000 | 6.111 | 0.000 | 6.549 | 1.000 | −0.318 |
| 482 | F214W | 0.000 | 3.063 | 0.000 | 3.968 | 0.146 | 0.548 |
| 483 | D215A | 0.000 | 6.464 | 0.000 | 6.753 | 0.000 | 1.091 |
| 484 | D215C | 0.000 | 6.825 | 0.000 | 7.307 | 1.000 | 0.290 |
| 485 | D215F | 0.000 | 6.885 | 0.000 | 6.289 | 0.038 | 0.654 |
| 486 | D215G | 0.000 | 7.790 | 0.000 | 7.567 | 1.000 | 0.196 |
| 487 | D215H | 0.000 | 7.049 | 0.000 | 7.023 | 1.000 | −0.170 |
| 488 | D215I | 0.000 | 5.673 | 0.000 | 6.954 | 1.000 | 0.264 |
| 489 | D215K | 0.000 | 7.484 | 0.000 | 6.864 | 1.000 | 0.225 |
| 490 | D215L | 0.000 | 7.574 | 0.000 | 8.072 | 1.000 | 0.288 |
| 491 | D215M | 0.000 | 7.413 | 0.000 | 7.618 | 0.000 | 1.163 |
| 492 | D215N | 0.000 | 8.518 | 0.000 | 6.838 | 1.000 | −1.043 |
| 493 | D215P | 0.000 | 7.738 | 0.000 | 7.326 | 1.000 | −0.344 |
| 494 | D215Q | 0.000 | 7.169 | 0.000 | 7.176 | 0.137 | 0.703 |
| 495 | D215R | 0.000 | 6.378 | 0.000 | 6.047 | 0.058 | 1.354 |
| 496 | D215S | 0.000 | 7.381 | 0.000 | 7.037 | 1.000 | 0.180 |
| 497 | D215T | 0.000 | 7.886 | 0.000 | 7.180 | 1.000 | 0.331 |
| 498 | D215V | 0.000 | 7.075 | 0.000 | 7.041 | 0.047 | 0.749 |
| 499 | D215W | 0.000 | 7.107 | 0.000 | 7.087 | 1.000 | 0.124 |
| 500 | D215Y | 0.000 | 7.759 | 0.000 | 7.376 | 1.000 | 0.266 |
| 501 | F216A | 0.073 | 2.385 | 0.000 | 3.237 | 1.000 | −0.500 |
| 502 | F216D | 0.082 | 0.916 | 0.000 | 2.042 | 1.000 | −0.701 |
| 503 | Y218A | 0.000 | 2.119 | 1.000 | 0.041 | 1.000 | −0.091 |
| 504 | Y218D | 0.000 | 4.937 | 0.000 | 5.573 | 1.000 | −0.052 |
| 505 | Y218E | 0.647 | 1.238 | 0.000 | 2.802 | 1.000 | −0.162 |
| 506 | Y218G | 0.000 | 3.172 | 0.000 | 4.660 | 1.000 | −1.317 |
| 507 | Y218L | 0.000 | 2.820 | 0.000 | 3.555 | 1.000 | 1.194 |
| 508 | Y218P | 0.000 | 3.667 | 0.000 | 5.706 | 1.000 | −1.733 |
| 509 | Y218S | 1.000 | −0.074 | 0.002 | 2.077 | 1.000 | −1.073 |
| 510 | Y218T | 0.004 | 1.500 | 0.000 | 2.770 | 1.000 | −0.834 |
| 511 | Y218W | 0.280 | 2.779 | 0.000 | 4.051 | 0.000 | 5.761 |
| 512 | L219P | 0.754 | 0.575 | 0.024 | 2.169 | 1.000 | −0.974 |
| 513 | L219R | 0.002 | 2.404 | 0.000 | 3.037 | 1.000 | −1.517 |
| 514 | L219V | 0.849 | 0.974 | 0.004 | 2.407 | 1.000 | 0.455 |
| 515 | K220I | 0.000 | 2.861 | 0.004 | 3.003 | 1.000 | −0.506 |
| 516 | K220V | 0.317 | 2.241 | 0.000 | 3.850 | 1.000 | −0.706 |
| 517 | C223A | 0.000 | 2.133 | 0.000 | 1.944 | 1.000 | 0.461 |
| 518 | C223E | 0.000 | 2.324 | 0.029 | 1.541 | 1.000 | 0.199 |
| 519 | C223P | 0.887 | 0.928 | 0.000 | 3.363 | 1.000 | −0.651 |
| 520 | K240W | 0.000 | 2.125 | 0.014 | 2.637 | 0.011 | 3.311 |
| 521 | K240Y | 0.000 | 2.251 | 0.000 | 1.241 | 0.815 | 0.474 |
| 522 | A249D | 0.605 | 0.785 | 0.000 | 2.239 | 1.000 | 0.464 |
| 523 | V250L | 0.001 | 3.118 | 0.000 | 3.821 | 1.000 | −0.620 |
| 524 | E251F | 0.091 | 1.854 | 0.001 | 2.147 | 0.009 | 1.763 |
| 525 | E251G | 0.764 | 0.866 | 0.002 | 2.138 | 0.688 | 1.462 |
| 526 | E251N | 0.188 | 1.376 | 0.000 | 2.387 | 0.464 | 1.108 |
| 527 | E251R | 0.014 | 2.292 | 0.002 | 2.349 | 0.272 | 1.407 |
| 528 | V252C | 0.003 | 2.007 | 0.000 | 2.015 | 0.685 | 0.566 |
| 529 | V252E | 0.011 | 1.870 | 0.000 | 2.035 | 0.002 | 1.046 |
| 530 | V252M | 0.004 | 2.215 | 0.000 | 2.109 | 1.000 | 0.037 |
| 531 | V252T | 0.002 | 2.719 | 0.253 | 0.953 | 1.000 | 0.506 |
| 532 | V252W | 0.281 | 1.147 | 0.001 | 2.560 | 1.000 | 0.229 |
| 533 | G254H | 0.000 | 3.441 | 0.024 | 1.823 | 1.000 | −0.136 |
| 534 | G254N | 0.001 | 2.997 | 0.001 | 2.546 | 1.000 | 0.715 |
| 535 | G254R | 0.049 | 1.865 | 0.000 | 2.182 | 0.001 | 2.021 |
| 536 | H257F | 0.000 | 3.268 | 0.000 | 4.862 | 1.000 | −0.429 |
| 537 | H257W | 0.489 | 1.475 | 0.002 | 2.618 | 1.000 | −0.193 |
| 538 | H257Y | 0.000 | 2.907 | 0.000 | 4.390 | 1.000 | −0.263 |
| 539 | D259A | 0.000 | 5.308 | 0.000 | 5.056 | 1.000 | −0.301 |
| 540 | D259C | 0.000 | 5.927 | 0.000 | 5.887 | 1.000 | 0.258 |
| 541 | D259F | 0.000 | 4.845 | 0.000 | 4.902 | 0.346 | 0.719 |
| 542 | D259G | 0.000 | 5.032 | 0.000 | 5.546 | 1.000 | −0.411 |
| 543 | D259H | 0.000 | 6.313 | 0.000 | 5.177 | 1.000 | −0.172 |
| 544 | D259I | 0.000 | 3.262 | 0.102 | 2.118 | 1.000 | −0.649 |
| 545 | D259K | 1.000 | 0.403 | 0.007 | 2.361 | 1.000 | −2.375 |
| 546 | D259L | 0.034 | 2.065 | 0.010 | 1.986 | 1.000 | −0.189 |
| 547 | D259M | 0.000 | 3.012 | 0.000 | 4.014 | 1.000 | −2.667 |
| 548 | D259N | 0.000 | 1.952 | 0.000 | 2.772 | 0.274 | 0.792 |
| 549 | D259P | 0.000 | 4.716 | 0.000 | 3.591 | 1.000 | −0.631 |
| 550 | D259Q | 0.003 | 2.989 | 0.000 | 3.822 | 1.000 | −0.492 |
| 551 | D259R | 0.009 | 1.793 | 0.000 | 2.373 | 1.000 | −1.324 |
| 552 | D259S | 0.000 | 4.876 | 0.000 | 5.579 | 1.000 | 0.276 |
| 553 | D259T | 0.000 | 4.652 | 0.000 | 5.727 | 1.000 | −0.003 |
| 554 | D259V | 0.010 | 2.745 | 0.000 | 3.569 | 1.000 | 0.207 |
| 555 | D259W | 0.000 | 5.321 | 0.000 | 4.814 | 1.000 | 0.413 |
| 556 | D259Y | 0.000 | 6.116 | 0.000 | 4.757 | 1.000 | 0.001 |
| 557 | L260W | 0.159 | 1.782 | 0.000 | 3.135 | 1.000 | −0.045 |
| 558 | Y261D | 0.044 | 2.909 | 0.000 | 3.875 | 1.000 | −2.287 |
| 559 | Y261E | 0.000 | 4.256 | 0.000 | 4.862 | 1.000 | −1.275 |
| 560 | Y261F | 0.618 | 1.396 | 1.000 | −0.135 | 0.001 | 4.482 |
| 561 | Y273D | 1.000 | 0.238 | 0.025 | 2.213 | 1.000 | −2.728 |
| 562 | Y273P | 1.000 | 0.442 | 0.001 | 2.289 | 1.000 | −1.693 |
| 563 | T274D | 0.006 | 2.538 | 0.005 | 2.477 | 1.000 | −0.448 |
| 564 | L275H | 0.012 | 1.971 | 0.000 | 2.830 | 1.000 | 0.204 |
| 565 | L275P | 0.014 | 2.741 | 0.072 | 2.195 | 1.000 | −1.922 |
| 566 | L275R | 0.000 | 4.908 | 0.000 | 4.126 | 1.000 | −0.028 |
| 567 | L275S | 0.008 | 2.108 | 0.118 | 1.258 | 1.000 | −0.294 |
| 568 | A277E | 1.000 | 0.054 | 0.000 | 2.362 | 1.000 | −0.176 |
| 569 | A277P | 0.000 | 2.310 | 0.000 | 3.163 | 1.000 | 0.313 |
| 570 | V278F | 0.274 | 1.147 | 0.000 | 3.281 | 1.000 | −0.649 |
| 571 | E280M | 1.000 | 0.528 | 1.000 | 0.337 | 0.003 | 4.937 |
| 572 | A281W | 0.225 | 1.569 | 0.000 | 2.050 | 1.000 | −0.588 |
| 573 | K287D | 0.000 | 2.846 | 0.000 | 2.825 | 1.000 | −2.251 |
| 574 | K287E | 0.000 | 3.660 | 0.000 | 2.842 | 1.000 | −2.842 |
| 575 | K287F | 0.003 | 3.030 | 0.000 | 4.088 | 1.000 | −0.279 |
| 576 | K287I | 0.000 | 1.882 | 0.000 | 2.562 | 1.000 | 0.218 |
| 577 | K287M | 0.979 | 0.948 | 0.000 | 2.883 | 1.000 | −0.776 |
| 578 | K287P | 0.000 | 2.040 | 0.000 | 2.639 | 1.000 | −3.586 |
| 579 | K287T | 0.001 | 2.180 | 0.000 | 1.774 | 0.040 | 0.969 |
| 580 | K287V | 0.030 | 2.588 | 0.000 | 3.123 | 1.000 | −1.327 |
| 581 | K289A | 0.000 | 4.107 | 0.000 | 4.306 | 1.000 | −0.476 |
| 582 | K289D | 0.000 | 1.760 | 0.000 | 2.319 | 1.000 | −1.614 |
| 583 | K289E | 0.000 | 2.591 | 0.094 | 2.120 | 1.000 | −1.234 |
| 584 | K289F | 0.000 | 4.137 | 0.000 | 4.863 | 1.000 | 0.227 |
| 585 | K289G | 0.000 | 5.298 | 0.001 | 3.462 | 1.000 | −1.610 |
| 586 | K289H | 0.000 | 3.286 | 0.000 | 4.079 | 0.027 | 0.902 |
| 587 | K289I | 0.000 | 3.094 | 0.000 | 3.199 | 0.149 | 0.767 |
| 588 | K289L | 0.000 | 4.265 | 0.000 | 4.234 | 1.000 | −0.253 |
| 589 | K289M | 0.000 | 3.412 | 0.000 | 3.670 | 1.000 | 0.207 |
| 590 | K289P | 0.000 | 4.561 | 0.012 | 2.835 | 1.000 | −0.249 |
| 591 | K289Q | 0.000 | 4.979 | 0.000 | 4.025 | 0.133 | 2.639 |
| 592 | K289R | 0.063 | 2.852 | 0.000 | 3.165 | 1.000 | 0.157 |
| 593 | K289S | 0.066 | 2.495 | 0.000 | 3.369 | 1.000 | −1.387 |
| 594 | K289T | 0.000 | 2.876 | 0.000 | 3.354 | 1.000 | 0.271 |
| 595 | K289V | 0.006 | 3.536 | 0.000 | 4.056 | 1.000 | −1.132 |
| 596 | K289W | 0.008 | 1.784 | 0.028 | 2.427 | 1.000 | −1.804 |
| 597 | K289Y | 0.000 | 2.750 | 0.000 | 3.145 | 0.118 | 0.657 |
| 598 | V290A | 1.000 | −0.663 | 0.015 | 2.495 | 1.000 | −1.703 |
| 599 | V290D | 0.000 | 3.176 | 0.000 | 3.501 | 1.000 | −2.243 |
| 600 | V290E | 0.000 | 3.143 | 0.000 | 3.265 | 1.000 | −1.567 |
| 601 | V290G | 0.000 | 4.599 | 0.000 | 5.179 | 1.000 | −0.871 |
| 602 | V290H | 0.000 | 3.476 | 0.000 | 4.090 | 1.000 | −1.347 |
| 603 | V290K | 0.000 | 3.153 | 0.000 | 3.370 | 1.000 | −0.665 |
| 604 | V290N | 0.006 | 2.770 | 0.000 | 3.594 | 1.000 | −1.909 |
| 605 | V290P | 0.000 | 6.006 | 0.000 | 5.064 | 1.000 | −1.608 |
| 606 | V290Q | 0.135 | 1.675 | 0.000 | 2.676 | 1.000 | −0.073 |
| 607 | V290R | 0.007 | 2.761 | 0.000 | 3.600 | 1.000 | −2.225 |
| 608 | V290S | 0.046 | 2.323 | 0.000 | 3.829 | 1.000 | −2.827 |
| 609 | V290T | 1.000 | 0.890 | 0.006 | 2.232 | 1.000 | −2.162 |
| 610 | A292D | 0.000 | 3.937 | 0.000 | 3.853 | 1.000 | −0.504 |
| 611 | A292E | 0.000 | 3.514 | 0.000 | 3.926 | 1.000 | 0.078 |
| 612 | A292N | 0.000 | 2.093 | 0.013 | 2.135 | 1.000 | −0.001 |
| 613 | A292Q | 0.290 | 2.251 | 0.000 | 4.653 | 1.000 | −0.448 |
| 614 | A292T | 0.242 | 2.298 | 0.004 | 3.782 | 1.000 | 0.480 |
| 615 | E294K | 1.000 | 0.563 | 0.000 | 2.810 | 1.000 | −2.474 |
| 616 | E294P | 0.000 | 3.711 | 0.000 | 3.756 | 1.000 | −2.858 |
| 617 | E294R | 1.000 | −0.606 | 0.002 | 2.107 | 1.000 | −2.907 |
| 618 | I295A | 0.022 | 1.868 | 0.000 | 2.560 | 1.000 | −1.891 |
| 619 | I295C | 0.570 | 0.777 | 0.000 | 2.731 | 1.000 | −2.539 |
| 620 | I295D | 0.000 | 4.417 | 0.000 | 3.200 | 1.000 | −1.733 |
| 621 | I295E | 0.000 | 4.104 | 0.000 | 3.517 | 1.000 | −2.412 |
| 622 | I295G | 0.000 | 3.456 | 0.000 | 3.315 | 1.000 | −2.581 |
| 623 | I295H | 0.000 | 3.041 | 0.000 | 3.119 | 1.000 | −1.186 |
| 624 | I295K | 0.000 | 5.739 | 0.000 | 5.462 | 1.000 | −1.289 |
| 625 | I295N | 0.000 | 3.610 | 0.001 | 2.266 | 1.000 | −2.578 |
| 626 | I295P | 0.000 | 4.560 | 0.000 | 3.890 | 1.000 | −2.183 |
| 627 | I295Q | 0.000 | 3.352 | 0.000 | 2.912 | 1.000 | −2.302 |
| 628 | I295R | 0.000 | 4.136 | 0.000 | 3.789 | 1.000 | −0.437 |
| 629 | I295S | 0.000 | 3.359 | 0.000 | 3.022 | 1.000 | −2.504 |
| 630 | I295T | 0.030 | 2.477 | 0.000 | 3.366 | 1.000 | −1.295 |
| 631 | I295W | 0.001 | 2.765 | 0.000 | 2.643 | 1.000 | −2.351 |
| 632 | I295Y | 0.000 | 3.435 | 0.002 | 2.525 | 1.000 | −1.666 |
| 633 | A298D | 0.343 | 1.345 | 0.002 | 2.189 | 1.000 | −1.961 |
| 634 | A298P | 0.000 | 2.937 | 0.006 | 2.000 | 1.000 | −2.697 |
| 635 | W299D | 0.000 | 3.507 | 0.000 | 2.768 | 1.000 | −2.797 |
| 636 | W299E | 0.000 | 2.934 | 0.000 | 3.038 | 1.000 | −2.433 |
| 637 | W299G | 0.047 | 1.698 | 0.006 | 2.191 | 1.000 | −1.424 |
| 638 | W299H | 0.459 | 1.426 | 0.026 | 2.112 | 1.000 | −2.800 |
| 639 | W299I | 0.044 | 2.242 | 0.022 | 2.133 | 1.000 | −1.691 |
| 640 | W299K | 0.000 | 5.438 | 0.000 | 4.797 | 1.000 | −1.689 |
| 641 | W299L | 0.023 | 2.285 | 0.000 | 3.125 | 1.000 | −2.216 |
| 642 | W299M | 0.056 | 1.836 | 0.000 | 2.428 | 1.000 | −1.017 |
| 643 | W299N | 0.000 | 2.422 | 0.000 | 3.226 | 1.000 | −2.101 |
| 644 | W299P | 0.000 | 3.895 | 0.000 | 3.670 | 1.000 | −1.588 |
| 645 | W299Q | 0.000 | 3.748 | 0.000 | 4.002 | 1.000 | −2.331 |
| 646 | W299R | 0.000 | 3.935 | 0.000 | 4.122 | 1.000 | −1.747 |
| 647 | W299T | 0.000 | 2.119 | 0.000 | 2.880 | 1.000 | −2.098 |
| 648 | W299V | 0.000 | 1.962 | 0.000 | 2.847 | 1.000 | −0.085 |
| 649 | E300P | 0.000 | 2.812 | 0.000 | 2.947 | 1.000 | −3.236 |
| 650 | T301P | 0.003 | 2.025 | 0.000 | 2.501 | 1.000 | −3.201 |
| 651 | G302P | 0.033 | 1.697 | 0.000 | 3.211 | 1.000 | −2.334 |
| 652 | L305H | 0.009 | 2.366 | 0.019 | 1.371 | 1.000 | −0.534 |
| 653 | V308D | 0.003 | 2.331 | 0.000 | 2.468 | 1.000 | −2.178 |
| 654 | V308F | 0.000 | 3.561 | 0.000 | 4.004 | 1.000 | −1.043 |
| 655 | V308H | 0.143 | 2.345 | 0.000 | 4.043 | 1.000 | −0.759 |
| 656 | V308K | 0.000 | 5.528 | 0.000 | 5.356 | 1.000 | −1.298 |
| 657 | V308L | 1.000 | −0.074 | 0.000 | 3.030 | 1.000 | −1.350 |
| 658 | V308N | 0.000 | 2.284 | 0.000 | 2.911 | 1.000 | −2.635 |
| 659 | V308Q | 0.000 | 3.562 | 0.000 | 3.518 | 1.000 | −1.249 |
| 660 | V308R | 0.000 | 4.693 | 0.000 | 5.265 | 1.000 | −2.058 |
| 661 | V308W | 0.000 | 4.488 | 0.000 | 3.885 | 1.000 | −2.090 |
| 662 | V308Y | 0.000 | 2.333 | 0.000 | 3.726 | 1.000 | −0.552 |
| 663 | A309D | 0.301 | 2.110 | 0.000 | 3.209 | 1.000 | −1.354 |
| 664 | A309P | 0.000 | 2.640 | 0.000 | 2.584 | 1.000 | 0.299 |
| 665 | A309R | 0.000 | 3.758 | 0.000 | 4.380 | 1.000 | −1.484 |
| 666 | Y311A | 0.000 | 5.781 | 0.000 | 5.925 | 1.000 | 0.007 |
| 667 | Y311C | 0.000 | 6.235 | 0.000 | 6.177 | 1.000 | −0.653 |
| 668 | Y311D | 0.007 | 3.780 | 0.000 | 3.840 | 1.000 | −1.225 |
| 669 | Y311E | 0.000 | 4.444 | 0.000 | 3.907 | 1.000 | −1.812 |
| 670 | Y311F | 0.000 | 5.877 | 0.000 | 4.837 | 1.000 | −0.077 |
| 671 | Y311G | 0.000 | 5.913 | 0.000 | 5.986 | 1.000 | −0.362 |
| 672 | Y311H | 0.000 | 3.914 | 0.000 | 5.023 | 1.000 | 0.380 |
| 673 | Y311I | 0.000 | 5.631 | 0.000 | 5.150 | 1.000 | −0.351 |
| 674 | Y311K | 0.000 | 4.308 | 0.000 | 5.586 | 0.096 | 0.601 |
| 675 | Y311L | 0.000 | 5.195 | 0.000 | 5.122 | 1.000 | −0.611 |
| 676 | Y311M | 0.000 | 5.019 | 0.000 | 4.997 | 1.000 | −0.601 |
| 677 | Y311N | 0.000 | 5.216 | 0.000 | 5.173 | 1.000 | −1.785 |
| 678 | Y311P | 0.000 | 6.932 | 0.000 | 5.212 | 1.000 | −0.638 |
| 679 | Y311Q | 0.000 | 5.540 | 0.000 | 5.067 | 1.000 | −0.205 |
| 680 | Y311R | 0.001 | 1.726 | 0.000 | 3.138 | 1.000 | −1.294 |
| 681 | Y311S | 0.000 | 6.057 | 0.000 | 5.799 | 1.000 | −0.278 |
| 682 | Y311T | 0.000 | 4.701 | 0.000 | 4.917 | 1.000 | −0.009 |
| 683 | Y311V | 0.000 | 5.790 | 0.000 | 5.597 | 1.000 | 0.156 |
| 684 | Y311W | 0.000 | 3.907 | 0.000 | 3.753 | 1.000 | 0.086 |
| 685 | S312D | 0.000 | 6.072 | 0.000 | 5.508 | 1.000 | −0.643 |
| 686 | S312E | 0.000 | 5.948 | 0.000 | 5.770 | 1.000 | 0.312 |
| 687 | S312F | 0.000 | 5.146 | 0.000 | 4.907 | 1.000 | −0.249 |
| 688 | S312H | 0.000 | 4.664 | 0.000 | 4.334 | 1.000 | −2.126 |
| 689 | S312I | 0.000 | 3.934 | 0.000 | 4.497 | 1.000 | 0.135 |
| 690 | S312K | 0.000 | 6.567 | 0.000 | 6.134 | 0.001 | 0.897 |
| 691 | S312L | 0.000 | 5.200 | 0.000 | 4.610 | 1.000 | −1.913 |
| 692 | S312M | 0.000 | 5.691 | 0.000 | 5.700 | 1.000 | −0.175 |
| 693 | S312N | 0.011 | 3.053 | 0.001 | 2.703 | 0.434 | 2.489 |
| 694 | S312P | 0.000 | 5.964 | 0.000 | 6.398 | 1.000 | −0.014 |
| 695 | S312Q | 0.000 | 6.409 | 0.000 | 5.958 | 0.464 | 0.421 |
| 696 | S312R | 0.000 | 4.809 | 0.000 | 4.796 | 1.000 | −2.559 |
| 697 | S312V | 0.000 | 4.035 | 0.000 | 4.604 | 1.000 | −0.105 |
| 698 | S312W | 0.000 | 5.692 | 0.000 | 5.553 | 1.000 | −0.196 |
| 699 | S312Y | 0.000 | 5.108 | 0.000 | 4.975 | 1.000 | −1.141 |
| 700 | M313P | 0.000 | 5.098 | 0.000 | 4.927 | 1.000 | −2.231 |
| 701 | M313Q | 0.152 | 1.783 | 0.015 | 2.493 | 0.531 | 1.466 |
| 702 | E314P | 0.000 | 5.589 | 0.000 | 5.483 | 1.000 | −2.002 |
| 703 | D315A | 0.000 | 6.364 | 0.000 | 5.825 | 0.371 | 0.584 |
| 704 | D315C | 0.000 | 4.506 | 0.000 | 5.897 | 0.134 | 0.672 |
| 705 | D315F | 0.000 | 5.173 | 0.000 | 4.809 | 0.088 | 1.726 |
| 706 | D315G | 0.000 | 5.720 | 0.000 | 6.295 | 0.042 | 3.050 |
| 707 | D315I | 0.000 | 5.627 | 0.000 | 5.304 | 1.000 | −1.543 |
| 708 | D315K | 0.000 | 3.257 | 0.121 | 2.056 | 1.000 | −1.073 |
| 709 | D315L | 0.000 | 6.056 | 0.000 | 5.281 | 1.000 | 0.052 |
| 710 | D315M | 0.000 | 6.660 | 0.000 | 5.414 | 1.000 | 0.107 |
| 711 | D315N | 0.000 | 8.102 | 0.000 | 7.171 | 1.000 | 0.042 |
| 712 | D315P | 0.000 | 5.373 | 0.000 | 5.359 | 1.000 | −1.100 |
| 713 | D315Q | 0.000 | 6.407 | 0.000 | 5.594 | 0.579 | 0.403 |
| 714 | D315R | 0.000 | 5.938 | 0.000 | 5.296 | 1.000 | −1.248 |
| 715 | D315S | 0.000 | 7.785 | 0.000 | 7.291 | 0.000 | 6.117 |
| 716 | D315T | 0.000 | 7.300 | 0.000 | 7.046 | 0.555 | 0.435 |
| 717 | D315V | 0.000 | 5.762 | 0.000 | 6.119 | 1.000 | −0.128 |
| 718 | D315W | 0.000 | 4.988 | 0.000 | 5.125 | 1.000 | 0.314 |
| 719 | D315Y | 0.000 | 6.027 | 0.000 | 3.936 | 1.000 | −0.268 |
| 720 | A316D | 0.000 | 2.516 | 0.006 | 1.674 | 1.000 | −2.110 |
| 721 | A316E | 0.007 | 2.922 | 0.000 | 3.509 | 1.000 | −2.088 |
| 722 | A316F | 0.034 | 2.319 | 0.000 | 2.325 | 1.000 | −3.408 |
| 723 | A316H | 0.000 | 3.917 | 0.000 | 3.806 | 1.000 | −1.370 |
| 724 | A316M | 0.333 | 0.907 | 0.005 | 2.238 | 1.000 | −1.992 |
| 725 | A316N | 0.015 | 2.291 | 0.026 | 1.494 | 1.000 | −2.306 |
| 726 | A316Q | 0.002 | 2.210 | 0.004 | 2.030 | 1.000 | −4.144 |
| 727 | A316R | 0.110 | 1.749 | 0.000 | 2.016 | 1.000 | −2.582 |
| 728 | A316W | 0.000 | 2.569 | 0.000 | 2.858 | 1.000 | −2.428 |
| 729 | A316Y | 0.000 | 2.880 | 0.001 | 2.354 | 1.000 | −1.377 |
| 730 | K317P | 0.000 | 3.228 | 0.000 | 3.471 | 1.000 | −2.081 |
| 731 | V318P | 0.000 | 2.965 | 0.000 | 3.982 | 1.000 | −2.109 |
| 732 | V318R | 0.212 | 1.510 | 0.001 | 2.219 | 1.000 | −1.389 |
| 733 | T319D | 0.015 | 1.490 | 0.000 | 4.574 | 1.000 | −1.281 |
| 734 | T319I | 0.061 | 3.026 | 0.000 | 3.729 | 1.000 | −0.075 |
| 735 | T319K | 0.000 | 6.057 | 0.000 | 6.076 | 1.000 | 0.250 |
| 736 | T319L | 0.120 | 1.726 | 0.000 | 4.008 | 1.000 | −0.669 |
| 737 | T319M | 0.000 | 3.132 | 0.000 | 3.511 | 1.000 | −0.100 |
| 738 | T319Q | 1.000 | 0.236 | 0.000 | 2.760 | 1.000 | 0.192 |
| 739 | T319R | 0.000 | 4.890 | 0.000 | 5.368 | 1.000 | −0.934 |
| 740 | T319V | 0.257 | 1.573 | 0.000 | 3.435 | 1.000 | −1.446 |

*adjusted p-val under dU/dI primer tolerance condition
**fitness_combined score under dU/dI primer tolerance condition
^{†}adjusted p-val under dU primer tolerance condition
††fitness_combined score under dU primer tolerance condition
^{‡}adjusted p-val under dUTP incorporation tolerance condition
^{‡‡}fitness_combined score under dUTP incorporation tolerance condition

### Example 2. Combinatorial Substitutions in KOD Polymerase Result in Improved dU/dI Template Tolerance, dU Template Tolerance, and/or dUTP Incorporation Tolerance

Combinatorial substitution KOD polymerase variants, relative to the wildtype sequence, were tested using compartmentalization self-replication (CSR) to identify combinations of substitutions that result in improved dU/dI primer tolerance, dU primer tolerance, and/or dUTP incorporation tolerance in KOD polymerase. Table 2 and Table 3 contain the amino acid (AA) substitutions (all in positions within the N-terminal domain of KOD polymerase) that were used to form the combinatorial substitution libraries. The CSR experiments were performed in the presence of dU and dI containing primers (4xdU_3xdI; measuring dU/dI primer tolerance), in the presence of dU containing primers (26xdU; measuring dU primer tolerance), or the presence of dUTP (U425T0; measuring dUTP incorporation tolerance).

TABLE 2
7p6s Library.

| | | AA | AA | AA | AA | AA |
|---|---|---|---|---|---|---|
| Posi- | WT | Substitu- | Substitu- | Substitu- | Substitu- | Substitu- |
| tion | residue | tion 1 | tion 2 | tion 3 | tion 4 | tion 5 |
| 7 | Y | C | D | L | M | P |
| 16 | I | F | H | L | M | W |
| 36 | P | A | H | R | S | W |
| 90 | P | F | I | S | W | Y |
| 93 | V | E | I | K | Q | R |
| 97 | R | A | F | H | P | W |
| 119 | R | E | M | P | Q | Y |

TABLE 3
15p2s Library.

| | WT | AA |
|---|---|---|
| Position | residue | substitution |
| 7 | Y | D |
| 14 | P | E |
| 16 | I | W |
| 36 | P | H |
| 37 | Y | D |
| 89 | H | D |
| 90 | P | F |
| 93 | V | K |
| 94 | P | D |
| 97 | R | F |
| 114 | I | P |
| 116 | F | P |
| 117 | A | M |
| 119 | R | Q |
| 123 | D | I |

Combined_fitness values were derived as described above. Variants were considered to have improved dU/dI primer tolerance, dU primer tolerance, or dUTP incorporation tolerance if they satisfied 2 criteria in the respective screen condition: (a) a fitness_combined score ≥2 and (b) an adjusted p-value p≤0.05. KOD polymerase variants that meet these criteria are statistically significantly improved in at least one of dU/dI primer tolerance, dU primer tolerance, or dUTP incorporation tolerance over wildtype KOD polymerase by four-fold or more.

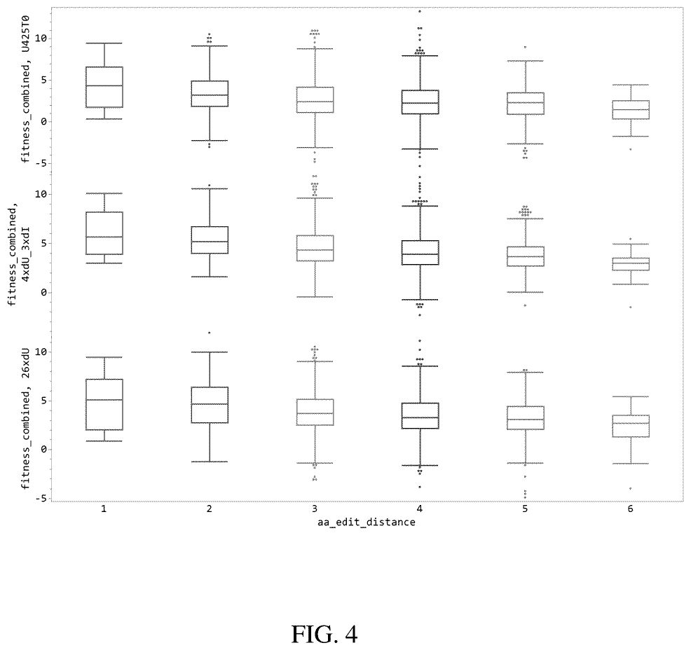

From the 7p6s library, which included more than 200,000 variants, 2470 combinatorial substitution variants were identified as having improved dU/dI primer tolerance, dU primer tolerance, or dUTP incorporation tolerance. The list of variants with improved dU/dI primer tolerance, dU primer tolerance, or dUTP incorporation tolerance is contained in Table 4 below. A summary of this data is shown in FIG. 4.

TABLE 4
7p6s Library variants with improved dU/dI primer tolerance, dU primer tolerance, or
dUTP incorporation tolerance.

| SEQ | | p-val | | p-val | | p-val | |
|---|---|---|---|---|---|---|---|
| ID NO | Substitutions | dI/dU* | dI/dU** | dU^{†} | dU^{††} | dUTP^{‡} | dUTP^{‡‡} |
| 93 | I16F | 0.000 | 6.900 | 0.000 | 6.081 | 0.000 | 6.307 |
| 741 | I16F_P36A | 0.000 | 5.850 | 0.000 | 1.650 | 0.000 | 1.690 |
| 742 | I16F_P36A_P90F | 0.000 | 6.840 | 0.000 | 4.920 | 0.000 | 5.900 |
| 743 | I16F_P36A_P90F_R97A | 0.140 | 3.010 | 0.010 | 2.620 | 1.000 | 0.100 |
| 744 | I16F_P36A_P90F_R97F | 0.000 | 3.700 | 0.700 | 2.160 | 0.080 | 2.100 |
| 745 | I16F_P36A_P90F_V93I | 0.000 | 7.670 | 0.000 | 7.050 | 0.000 | 7.410 |
| 746 | I16F_P36A_P90F_V93I_R97A | 0.000 | 4.930 | 0.000 | 5.870 | 0.090 | 3.330 |
| 747 | I16F_P36A_P90F_V93I_R97F | 0.000 | 3.460 | 0.000 | 3.450 | 1.000 | 1.860 |
| 748 | I16F_P36A_P90F_V93I_R97H | 0.000 | 4.800 | 0.000 | 5.030 | 0.320 | 3.140 |
| 749 | I16F_P36A_P90F_V93R_R97A | 0.000 | 2.360 | 0.000 | 2.430 | 0.000 | 2.590 |
| 750 | I16F_P36A_P90I | 0.000 | 6.600 | 0.000 | 7.090 | 0.000 | 7.560 |
| 751 | I16F_P36A_P90I_R97A | 0.000 | 3.500 | 0.000 | 3.130 | 0.000 | 2.990 |
| 752 | I16F_P36A_P90I_R97F | 0.000 | 3.770 | 0.000 | 3.830 | 0.000 | 3.350 |
| 753 | I16F_P36A_P90I_R97H | 0.000 | 4.670 | 0.040 | 3.250 | 1.000 | 1.920 |
| 754 | I16F_P36A_P90I_V93I | 0.000 | 6.340 | 0.010 | 5.900 | 0.000 | 7.600 |
| 755 | I16F_P36A_P90I_V93I_R97A | 1.000 | 3.200 | 0.010 | 5.130 | 0.010 | 3.210 |
| 756 | I16F_P36A_P90I_V93I_R97F | 0.000 | 5.220 | 0.000 | 5.610 | 0.440 | 3.170 |
| 757 | I16F_P36A_P90I_V93I_R97H | 0.000 | 4.610 | 0.000 | 4.260 | 0.000 | 4.090 |
| 758 | I16F_P36A_P90I_V93I_R97W | 0.000 | 4.040 | 0.000 | 3.020 | 0.130 | 3.190 |
| 759 | I16F_P36A_P90S_V93I | 0.000 | 2.870 | 1.000 | −0.970 | 1.000 | 0.710 |
| 760 | I16F_P36A_P90S_V93R_R97A | 0.000 | 2.550 | 1.000 | 0.680 | 1.000 | 1.900 |
| 761 | I16F_P36A_P90W | 0.000 | 6.950 | 0.000 | 6.390 | 0.000 | 5.830 |
| 762 | I16F_P36A_P90W_R97W | 0.000 | 2.030 | 1.000 | 2.510 | 0.710 | 1.380 |
| 763 | I16F_P36A_P90W_V93I | 0.000 | 7.410 | 0.000 | 7.320 | 0.000 | 6.520 |
| 764 | I16F_P36A_P90W_V93I_R97A | 0.010 | 3.600 | 0.000 | 5.360 | 0.090 | 3.060 |
| 765 | I16F_P36A_P90W_V93I_R97F | 0.000 | 3.020 | 0.000 | 4.430 | 1.000 | 1.760 |
| 766 | I16F_P36A_P90W_V93I_R97H | 0.220 | 4.220 | 0.000 | 3.390 | 0.010 | 3.440 |
| 767 | I16F_P36A_P90W_V93I_R97W | 0.000 | 5.380 | 0.000 | 5.110 | 0.000 | 4.630 |
| 768 | I16F_P36A_P90W_V93Q | 0.000 | 3.730 | 0.000 | 3.860 | 0.010 | 4.090 |
| 769 | I16F_P36A_P90W_V93R_R97A | 0.000 | 4.250 | 0.000 | 4.690 | 0.000 | 4.410 |
| 770 | I16F_P36A_P90Y | 0.000 | 5.520 | 0.000 | 5.950 | 0.000 | 6.550 |
| 771 | I16F_P36A_P90Y_R97A | 0.000 | 5.700 | 0.000 | 4.960 | 0.000 | 3.610 |
| 772 | I16F_P36A_P90Y_R97H | 0.000 | 3.770 | 1.000 | 0.610 | 1.000 | 0.550 |
| 773 | I16F_P36A_P90Y_V93I | 0.000 | 6.320 | 0.000 | 5.600 | 0.000 | 6.450 |
| 774 | I16F_P36A_P90Y_V93I_R97A | 0.000 | 4.800 | 1.000 | 1.710 | 1.000 | 2.180 |
| 775 | I16F_P36A_P90Y_V93I_R97F | 0.080 | 3.080 | 0.000 | 3.940 | 1.000 | 1.750 |
| 776 | I16F_P36A_P90Y_V93I_R97H | 0.000 | 4.540 | 1.000 | 2.000 | 0.040 | 3.490 |
| 777 | I16F_P36A_P90Y_V93I_R97W | 0.000 | 3.100 | 0.000 | 2.630 | 1.000 | 0.670 |
| 778 | I16F_P36A_R97A | 0.000 | 6.500 | 0.000 | 5.360 | 0.000 | 5.060 |
| 779 | I16F_P36A_R97F | 0.000 | 7.650 | 0.000 | 5.890 | 0.000 | 6.510 |
| 780 | I16F_P36A_R97H | 0.000 | 6.390 | 0.000 | 5.400 | 0.000 | 5.960 |
| 781 | I16F_P36A_R97W | 0.000 | 5.650 | 0.000 | 5.630 | 0.000 | 6.320 |
| 782 | I16F_P36A_V93E | 0.000 | 3.640 | 0.000 | 2.950 | 0.000 | 3.030 |
| 783 | I16F_P36A_V93I | 0.000 | 4.120 | 0.200 | 1.040 | 0.050 | 1.400 |
| 784 | I16F_P36A_V93I_R97A | 0.000 | 6.320 | 0.000 | 5.630 | 0.000 | 6.260 |
| 785 | I16F_P36A_V93I_R97F | 0.000 | 6.940 | 0.000 | 5.210 | 0.000 | 5.580 |
| 786 | I16F_P36A_V93I_R97H | 0.000 | 6.240 | 0.000 | 3.470 | 0.030 | 3.460 |
| 787 | I16F_P36A_V93I_R97W | 0.000 | 6.780 | 0.000 | 4.420 | 0.000 | 4.670 |
| 788 | I16F_P36A_V93K | 0.030 | 3.460 | 0.000 | 4.150 | 1.000 | 1.390 |
| 789 | I16F_P36A_V93K_R97F | 0.000 | 2.750 | 0.010 | 2.860 | 1.000 | 0.940 |
| 790 | I16F_P36A_V93Q | 0.000 | 6.930 | 0.000 | 7.240 | 0.000 | 6.920 |
| 791 | I16F_P36A_V93Q_R97A | 0.000 | 6.370 | 0.000 | 3.510 | 0.850 | 2.800 |
| 792 | I16F_P36A_V93Q_R97F | 0.000 | 3.780 | 0.440 | 3.100 | 0.000 | 3.650 |
| 793 | I16F_P36A_V93Q_R97W | 0.000 | 3.110 | 0.000 | 3.230 | 1.000 | 1.490 |
| 794 | I16F_P36A_V93R | 0.000 | 6.540 | 0.000 | 6.880 | 0.010 | 3.620 |
| 795 | I16F_P36A_V93R_R97A | 0.000 | 6.420 | 0.000 | 6.470 | 0.000 | 4.180 |
| 796 | I16F_P36A_V93R_R97A_R119Q | 0.000 | 4.380 | 1.000 | 1.580 | 1.000 | 0.940 |
| 797 | I16F_P36A_V93R_R97F | 0.020 | 6.320 | 0.000 | 5.830 | 0.540 | 4.000 |
| 798 | I16F_P36A_V93R_R97H | 1.000 | 2.190 | 0.010 | 2.640 | 0.000 | 4.230 |
| 799 | I16F_P36A_V93R_R97W | 0.510 | 2.130 | 0.000 | 4.180 | 1.000 | −0.690 |
| 800 | I16F_P36H | 0.000 | 8.670 | 0.000 | 8.150 | 0.000 | 6.520 |
| 801 | I16F_P36H_P90F | 0.000 | 2.310 | 0.020 | 1.300 | 1.000 | 0.420 |
| 802 | I16F_P36H_P90F_V93I | 0.000 | 2.560 | 1.000 | 0.530 | 1.000 | 0.030 |
| 803 | I16F_P36H_P90W | 0.020 | 2.580 | 1.000 | 0.630 | 1.000 | 1.060 |
| 804 | I16F_P36H_P90W_R97H | 0.000 | 3.950 | 0.000 | 2.960 | 0.080 | 1.980 |
| 805 | I16F_P36H_P90Y_R97A | 0.000 | 3.210 | 0.000 | 3.070 | 0.100 | 1.700 |
| 806 | I16F_P36H_R97A | 0.000 | 6.580 | 0.000 | 5.020 | 1.000 | 1.710 |
| 807 | I16F_P36H_R97F | 0.000 | 5.330 | 0.000 | 4.100 | 0.160 | 3.870 |
| 808 | I16F_P36H_R97H | 0.100 | 3.770 | 0.000 | 4.930 | 1.000 | 2.260 |
| 809 | I16F_P36H_R119Q | 0.000 | 3.810 | 0.370 | 2.620 | 0.000 | 2.550 |
| 810 | I16F_P36H_V93I | 0.000 | 7.560 | 0.000 | 7.850 | 0.000 | 6.380 |
| 811 | I16F_P36H_V93I_R97A | 0.000 | 5.310 | 0.000 | 4.620 | 0.000 | 4.640 |
| 812 | I16F_P36H_V93I_R97F | 0.000 | 4.930 | 0.000 | 4.360 | 0.000 | 4.170 |
| 813 | I16F_P36H_V93I_R97H | 0.220 | 3.690 | 0.000 | 4.610 | 1.000 | 0.590 |
| 814 | I16F_P36H_V93Q | 1.000 | 0.390 | 0.040 | 2.610 | 1.000 | 0.640 |
| 815 | I16F_P36R | 0.000 | 7.270 | 0.000 | 5.510 | 1.000 | 3.280 |
| 816 | I16F_P36R_R97A | 0.000 | 6.160 | 0.000 | 6.290 | 0.000 | 5.240 |
| 817 | I16F_P36R_R97F | 0.000 | 3.440 | 0.010 | 4.310 | 1.000 | 2.040 |
| 818 | I16F_P36R_R97H | 0.000 | 5.320 | 0.000 | 5.650 | 1.000 | 2.620 |
| 819 | I16F_P36S | 0.000 | 3.900 | 1.000 | 0.440 | 1.000 | 0.380 |
| 820 | I16F_P36S_P90F_R97A | 0.000 | 2.200 | 1.000 | 0.880 | 1.000 | −1.690 |
| 821 | I16F_P36S_P90F_V93I | 0.000 | 4.080 | 0.000 | 3.640 | 0.000 | 3.900 |
| 822 | I16F_P36S_P90F_V93I_R97A | 0.000 | 3.280 | 0.000 | 1.970 | 0.000 | 2.300 |
| 823 | I16F_P36S_P90I_V93I | 0.200 | 3.480 | 0.000 | 2.040 | 0.010 | 3.390 |
| 824 | I16F_P36S_P90I_V93I_R97F | 0.070 | 1.950 | 0.060 | 1.620 | 0.010 | 2.100 |
| 825 | I16F_P36S_P90W_R97A | 0.000 | 2.000 | 1.000 | −1.620 | 1.000 | −2.460 |
| 826 | I16F_P36S_P90Y | 0.000 | 2.690 | 0.080 | 1.800 | 0.110 | 1.760 |
| 827 | I16F_P36S_R97A | 0.000 | 4.620 | 0.000 | 4.520 | 0.000 | 3.890 |
| 828 | I16F_P36S_R97F | 0.140 | 3.950 | 0.000 | 4.320 | 1.000 | 0.540 |
| 829 | I16F_P36S_R97H | 0.040 | 3.660 | 1.000 | 2.530 | 1.000 | 3.920 |
| 830 | I16F_P36S_V93I | 0.000 | 3.720 | 1.000 | −0.040 | 1.000 | 0.440 |
| 831 | I16F_P36S_V93I_R97F | 0.000 | 5.350 | 1.000 | 1.590 | 1.000 | 2.730 |
| 832 | I16F_P36S_V93I_R97W | 0.000 | 4.800 | 1.000 | 3.330 | 1.000 | −0.480 |
| 833 | I16F_P36S_V93Q | 0.000 | 5.730 | 0.730 | 2.510 | 1.000 | 3.350 |
| 834 | I16F_P36S_V93R_R97F | 0.000 | 2.400 | 1.000 | 0.410 | 1.000 | −3.320 |
| 835 | I16F_P36S_V93R_R97H | 0.470 | 1.860 | 1.000 | 1.770 | 0.010 | 2.270 |
| 836 | I16F_P36W | 0.000 | 4.450 | 0.030 | 3.380 | 0.000 | 5.150 |
| 837 | I16F_P36W_P90F_V93I | 0.000 | 3.810 | 0.010 | 3.480 | 0.010 | 2.810 |
| 838 | I16F_P36W_P90F_V93I_R97A | 0.000 | 3.170 | 1.000 | 2.260 | 1.000 | 1.530 |
| 839 | I16F_P36W_P90F_V93I_R97F | 0.000 | 3.280 | 0.000 | 3.260 | 0.000 | 2.820 |
| 840 | I16F_P36W_P90F_V93K_R97A | 0.000 | 2.080 | 1.000 | −0.120 | 1.000 | −2.350 |
| 841 | I16F_P36W_P90F_V93R_R97A | 0.000 | 2.850 | 0.000 | 2.440 | 1.000 | −0.570 |
| 842 | I16F_P36W_P90I_V93I | 0.000 | 2.840 | 0.000 | 1.780 | 1.000 | 0.300 |
| 843 | I16F_P36W_P90I_V93R_R97F | 0.000 | 3.220 | 1.000 | −0.640 | 1.000 | −3.170 |
| 844 | I16F_P36W_P90W_V93I | 0.010 | 3.180 | 0.010 | 2.060 | 1.000 | 0.510 |
| 845 | I16F_P36W_P90Y_V93K_R97F | 0.000 | 2.330 | 1.000 | −4.290 | 1.000 | −1.790 |
| 846 | I16F_P36W_R97A | 0.000 | 4.420 | 0.000 | 2.610 | 1.000 | 1.910 |
| 847 | I16F_P36W_R97F | 1.000 | 1.260 | 0.010 | 2.310 | 1.000 | 0.090 |
| 848 | I16F_P36W_V93I | 0.000 | 4.860 | 0.000 | 6.010 | 0.000 | 5.150 |
| 849 | I16F_P36W_V93I_R97A | 0.040 | 3.070 | 1.000 | 2.710 | 1.000 | 0.890 |
| 850 | I16F_P90F | 0.000 | 10.290 | 0.000 | 9.960 | 0.000 | 10.070 |
| 851 | I16F_P90F_R97A | 0.000 | 7.580 | 0.000 | 9.080 | 0.000 | 5.810 |
| 852 | I16F_P90F_R97F | 0.000 | 6.360 | 0.000 | 7.260 | 0.000 | 6.530 |
| 853 | I16F_P90F_R97H | 0.000 | 7.110 | 0.000 | 5.980 | 0.000 | 4.760 |
| 854 | I16F_P90F_R97W | 0.000 | 4.610 | 0.000 | 4.870 | 0.000 | 3.700 |
| 855 | I16F_P90F_V93I | 0.000 | 10.880 | 0.000 | 10.230 | 0.000 | 11.030 |
| 856 | I16F_P90F_V93I_R97A | 0.000 | 9.320 | 0.000 | 8.540 | 0.000 | 7.480 |
| 857 | I16F_P90F_V93I_R97F | 0.000 | 8.800 | 0.000 | 9.300 | 0.000 | 7.480 |
| 858 | I16F_P90F_V93I_R97H | 0.000 | 7.930 | 0.000 | 8.040 | 0.000 | 7.510 |
| 859 | I16F_P90F_V93I_R97W | 0.000 | 5.780 | 0.000 | 6.040 | 0.000 | 5.630 |
| 860 | I16F_P90F_V93K | 0.000 | 4.710 | 0.010 | 3.660 | 0.010 | 3.590 |
| 861 | I16F_P90F_V93K_R97A | 0.000 | 5.300 | 1.000 | 2.200 | 1.000 | 2.280 |
| 862 | I16F_P90F_V93K_R97F | 0.000 | 3.550 | 0.400 | 2.530 | 1.000 | 1.580 |
| 863 | I16F_P90F_V93K_R97H | 0.000 | 3.380 | 1.000 | 2.900 | 1.000 | 2.310 |
| 864 | I16F_P90F_V93Q | 0.000 | 7.600 | 0.000 | 5.870 | 0.000 | 4.240 |
| 865 | I16F_P90F_V93Q_R97A | 0.000 | 5.420 | 0.000 | 5.790 | 0.000 | 3.720 |
| 866 | I16F_P90F_V93Q_R97F | 1.000 | 2.920 | 0.000 | 3.880 | 1.000 | 1.840 |
| 867 | I16F_P90F_V93Q_R97H | 0.000 | 3.440 | 0.000 | 5.470 | 0.680 | 2.000 |
| 868 | I16F_P90F_V93Q_R97W | 0.000 | 2.540 | 0.890 | 2.550 | 0.000 | 2.550 |
| 869 | I16F_P90F_V93R | 0.000 | 6.060 | 0.000 | 6.050 | 0.040 | 3.240 |
| 870 | I16F_P90F_V93R_R97A | 0.000 | 5.570 | 0.000 | 4.200 | 0.270 | 2.310 |
| 871 | I16F_P90F_V93R_R97F | 0.000 | 5.200 | 0.000 | 3.540 | 0.010 | 3.210 |
| 872 | I16F_P90F_V93R_R97H | 0.000 | 4.630 | 0.130 | 2.200 | 1.000 | 1.400 |
| 873 | I16F_P90F_V93R_R97P | 1.000 | 1.700 | 1.000 | 1.020 | 0.000 | 2.150 |
| 874 | I16F_P90I | 0.000 | 8.210 | 0.000 | 8.560 | 0.000 | 9.690 |
| 875 | I16F_P90I_R97A | 0.000 | 8.030 | 0.000 | 7.830 | 0.000 | 5.740 |
| 876 | I16F_P90I_R97F | 0.000 | 7.780 | 0.000 | 7.820 | 0.000 | 6.370 |
| 877 | I16F_P90I_R97H | 0.000 | 7.660 | 0.000 | 6.800 | 0.000 | 4.620 |
| 878 | I16F_P90I_R97P | 0.000 | 2.700 | 0.320 | 1.760 | 1.000 | −1.550 |
| 879 | I16F_P90I_R97W | 0.000 | 7.270 | 0.000 | 6.150 | 0.000 | 5.020 |
| 880 | I16F_P90I_V93E | 0.000 | 2.340 | 0.010 | 2.150 | 0.000 | 1.620 |
| 881 | I16F_P90I_V93I | 0.000 | 10.980 | 0.000 | 10.270 | 0.000 | 10.470 |
| 882 | I16F_P90I_V93I_R97A | 0.000 | 7.720 | 0.000 | 7.940 | 0.000 | 6.010 |
| 883 | I16F_P90I_V93I_R97F | 0.000 | 6.780 | 0.000 | 5.960 | 0.000 | 6.620 |
| 884 | I16F_P90I_V93I_R97H | 0.000 | 7.100 | 0.000 | 7.490 | 0.000 | 6.540 |
| 885 | I16F_P90I_V93I_R97P | 1.000 | −0.730 | 0.000 | 2.020 | 1.000 | −2.230 |
| 886 | I16F_P90I_V93I_R97W | 0.000 | 7.500 | 0.000 | 7.530 | 0.000 | 5.410 |
| 887 | I16F_P90I_V93K | 0.000 | 7.110 | 0.000 | 5.900 | 0.000 | 5.300 |
| 888 | I16F_P90I_V93K_R97A | 0.000 | 3.380 | 1.000 | 1.530 | 1.000 | 2.030 |
| 889 | I16F_P90I_V93K_R97F | 0.000 | 5.140 | 0.000 | 5.510 | 1.000 | 2.120 |
| 890 | I16F_P90I_V93K_R97W | 0.000 | 4.360 | 0.000 | 3.620 | 0.490 | 2.810 |
| 891 | I16F_P90I_V93Q | 0.000 | 7.890 | 0.000 | 5.220 | 0.000 | 5.200 |
| 892 | I16F_P90I_V93Q_R97A | 0.000 | 4.570 | 0.000 | 4.490 | 0.000 | 4.660 |
| 893 | I16F_P90I_V93Q_R97F | 0.030 | 4.090 | 1.000 | 2.430 | 1.000 | 1.470 |
| 894 | I16F_P90I_V93Q_R97H | 0.000 | 3.160 | 1.000 | 1.540 | 1.000 | 1.300 |
| 895 | I16F_P90I_V93R | 0.000 | 7.360 | 0.000 | 5.690 | 0.000 | 5.710 |
| 896 | I16F_P90I_V93R_R97A | 0.000 | 6.690 | 0.000 | 5.890 | 0.000 | 6.250 |
| 897 | I16F_P90I_V93R_R97A_R119Q | 0.000 | 2.400 | 1.000 | −1.040 | 1.000 | 0.340 |
| 898 | I16F_P90I_V93R_R97F | 0.000 | 4.420 | 0.000 | 3.980 | 1.000 | 1.330 |
| 899 | I16F_P90I_V93R_R97H | 0.000 | 5.990 | 0.020 | 3.310 | 0.000 | 3.850 |
| 900 | I16F_P90I_V93R_R97W | 0.000 | 6.190 | 0.000 | 4.820 | 0.050 | 3.080 |
| 901 | I16F_P90S | 0.000 | 5.570 | 1.000 | 0.270 | 1.000 | 0.500 |
| 902 | I16F_P90S_R97A | 0.000 | 5.220 | 0.000 | 4.500 | 0.000 | 6.080 |
| 903 | I16F_P90S_R97F | 0.000 | 4.700 | 0.000 | 4.440 | 0.000 | 6.170 |
| 904 | I16F_P90S_R97W | 0.000 | 5.260 | 0.000 | 4.300 | 0.000 | 5.270 |
| 905 | I16F_P90S_V93I | 0.000 | 4.580 | 0.000 | 1.770 | 0.000 | 2.610 |
| 906 | I16F_P90S_V93I_R97A | 0.000 | 6.910 | 0.000 | 5.540 | 0.000 | 6.200 |
| 907 | I16F_P90S_V93I_R97F | 0.000 | 6.900 | 0.000 | 5.830 | 0.000 | 6.540 |
| 908 | I16F_P90S_V93I_R97H | 0.000 | 6.870 | 0.000 | 5.200 | 0.000 | 5.940 |
| 909 | I16F_P90S_V93I_R97W | 0.000 | 5.610 | 0.000 | 4.870 | 0.000 | 6.040 |
| 910 | I16F_P90S_V93Q | 1.000 | 1.490 | 0.000 | 2.460 | 0.030 | 3.530 |
| 911 | I16F_P90S_V93R | 0.000 | 4.910 | 0.000 | 4.740 | 0.000 | 2.900 |
| 912 | I16F_P90S_V93R_R97F | 0.000 | 4.290 | 1.000 | 2.390 | 1.000 | −1.340 |
| 913 | I16F_P90S_V93R_R97H | 0.000 | 2.570 | 0.000 | 1.580 | 0.000 | 2.220 |
| 914 | I16F_P90S_V93R_R97W | 0.000 | 3.370 | 0.000 | 4.000 | 0.040 | 3.260 |
| 915 | I16F_P90W | 0.000 | 10.530 | 0.000 | 8.830 | 0.000 | 10.520 |
| 916 | I16F_P90W_R97A | 0.000 | 6.800 | 0.000 | 6.420 | 0.000 | 5.720 |
| 917 | I16F_P90W_R97F | 0.000 | 6.410 | 0.000 | 6.080 | 0.000 | 4.970 |
| 918 | I16F_P90W_R97H | 0.000 | 6.500 | 0.000 | 6.550 | 0.000 | 5.680 |
| 919 | I16F_P90W_R97W | 0.000 | 5.470 | 0.000 | 5.610 | 0.000 | 5.040 |
| 920 | I16F_P90W_V93E_R97F | 0.000 | 2.710 | 0.010 | 2.310 | 0.000 | 2.710 |
| 921 | I16F_P90W_V93I | 0.000 | 10.510 | 0.000 | 9.910 | 0.000 | 10.500 |
| 922 | I16F_P90W_V93I_R97A | 0.000 | 8.280 | 0.000 | 8.010 | 0.000 | 5.500 |
| 923 | I16F_P90W_V93I_R97F | 0.000 | 6.630 | 0.000 | 6.580 | 0.000 | 4.900 |
| 924 | I16F_P90W_V93I_R97H | 0.000 | 7.310 | 0.000 | 7.520 | 0.000 | 6.300 |
| 925 | I16F_P90W_V93I_R97W | 0.000 | 6.760 | 0.000 | 5.430 | 0.000 | 5.750 |
| 926 | I16F_P90W_V93K | 0.000 | 4.680 | 0.000 | 4.090 | 0.000 | 4.010 |
| 927 | I16F_P90W_V93K_R97F | 0.000 | 3.760 | 0.510 | 2.940 | 0.250 | 3.220 |
| 928 | I16F_P90W_V93K_R97W | 0.000 | 5.310 | 0.000 | 4.130 | 0.000 | 4.650 |
| 929 | I16F_P90W_V93Q | 0.000 | 6.240 | 0.000 | 6.150 | 0.000 | 4.320 |
| 930 | I16F_P90W_V93Q_R97A | 0.000 | 6.900 | 0.000 | 4.480 | 0.000 | 4.820 |
| 931 | I16F_P90W_V93Q_R97F | 0.000 | 3.500 | 1.000 | 1.560 | 1.000 | 2.660 |
| 932 | I16F_P90W_V93Q_R97W | 0.000 | 3.550 | 1.000 | 1.490 | 1.000 | 1.670 |
| 933 | I16F_P90W_V93R | 0.000 | 5.310 | 0.010 | 4.410 | 0.140 | 3.310 |
| 934 | I16F_P90W_V93R_R97A | 0.010 | 2.980 | 0.000 | 3.920 | 0.000 | 3.180 |
| 935 | I16F_P90W_V93R_R97H | 0.280 | 3.380 | 0.010 | 3.160 | 0.180 | 2.790 |
| 936 | I16F_P90W_V93R_R97W | 0.000 | 4.160 | 0.000 | 4.350 | 1.000 | 1.240 |
| 937 | I16F_P90Y | 0.000 | 9.910 | 0.000 | 9.100 | 0.000 | 10.050 |
| 938 | I16F_P90Y_R97A | 0.000 | 7.180 | 0.000 | 7.560 | 0.000 | 4.680 |
| 939 | I16F_P90Y_R97F | 0.000 | 7.000 | 0.000 | 7.010 | 0.000 | 6.010 |
| 940 | I16F_P90Y_R97H | 0.000 | 4.400 | 0.000 | 4.160 | 0.000 | 4.300 |
| 941 | I16F_P90Y_V93I | 0.000 | 10.810 | 0.000 | 9.400 | 0.000 | 10.620 |
| 942 | I16F_P90Y_V93I_R97A | 0.000 | 8.240 | 0.000 | 7.780 | 0.000 | 6.950 |
| 943 | I16F_P90Y_V93I_R97F | 0.000 | 6.060 | 0.000 | 7.090 | 0.000 | 4.640 |
| 944 | I16F_P90Y_V93I_R97H | 0.000 | 7.370 | 0.000 | 7.650 | 0.000 | 6.720 |
| 945 | I16F_P90Y_V93I_R97W | 0.000 | 7.990 | 0.060 | 4.900 | 0.000 | 4.970 |
| 946 | I16F_P90Y_V93K_R97A | 0.000 | 3.290 | 0.000 | 3.620 | 1.000 | 0.130 |
| 947 | I16F_P90Y_V93K_R97F | 0.000 | 3.310 | 0.650 | 2.190 | 1.000 | −0.130 |
| 948 | I16F_P90Y_V93Q | 0.000 | 5.570 | 0.000 | 6.500 | 0.000 | 4.520 |
| 949 | I16F_P90Y_V93Q_R97A | 0.000 | 5.050 | 0.000 | 4.940 | 0.020 | 2.850 |
| 950 | I16F_P90Y_V93Q_R97F | 0.000 | 3.650 | 1.000 | 2.210 | 1.000 | −1.180 |
| 951 | I16F_P90Y_V93Q_R97H | 0.650 | 2.710 | 0.020 | 5.090 | 0.040 | 2.330 |
| 952 | I16F_P90Y_V93Q_R97W | 0.000 | 2.560 | 1.000 | 1.670 | 1.000 | 0.720 |
| 953 | I16F_P90Y_V93R | 0.000 | 4.720 | 0.240 | 3.080 | 0.020 | 2.670 |
| 954 | I16F_P90Y_V93R_R97A | 0.000 | 4.910 | 1.000 | 3.150 | 1.000 | 1.500 |
| 955 | I16F_P90Y_V93R_R97F | 0.000 | 4.150 | 1.000 | 1.280 | 1.000 | 0.270 |
| 956 | I16F_R97A | 0.000 | 7.890 | 0.000 | 4.220 | 0.000 | 5.190 |
| 957 | I16F_R97A_R119M | 0.010 | 3.920 | 1.000 | 1.430 | 1.000 | 0.540 |
| 958 | I16F_R97F | 0.000 | 7.420 | 0.000 | 6.920 | 0.000 | 7.810 |
| 959 | I16F_R97F_R119Q | 0.000 | 5.380 | 0.000 | 3.920 | 0.000 | 5.200 |
| 960 | I16F_R97H | 0.000 | 7.460 | 0.000 | 5.660 | 0.000 | 6.200 |
| 961 | I16F_R97W | 0.000 | 4.340 | 0.000 | 2.900 | 0.000 | 2.620 |
| 962 | I16F_R119Q | 0.000 | 4.770 | 0.870 | 2.040 | 1.000 | 2.300 |
| 963 | I16F_V93E | 0.000 | 7.640 | 0.000 | 6.850 | 0.000 | 6.920 |
| 964 | I16F_V93E_R97A | 0.000 | 3.400 | 0.000 | 4.660 | 0.010 | 3.530 |
| 965 | I16F_V93E_R97F | 1.000 | 1.540 | 0.000 | 3.140 | 1.000 | 1.880 |
| 966 | I16F_V93E_R97H | 1.000 | 1.040 | 0.000 | 3.420 | 1.000 | 2.130 |
| 967 | I16F_V93I | 0.000 | 6.710 | 0.000 | 5.060 | 0.000 | 5.520 |
| 968 | I16F_V93I_R97A | 0.000 | 8.300 | 0.000 | 4.700 | 0.000 | 4.130 |
| 969 | I16F_V93I_R97F | 0.000 | 9.840 | 0.000 | 9.560 | 0.000 | 9.530 |
| 970 | I16F_V93I_R97F_R119Q | 0.000 | 4.000 | 0.000 | 3.060 | 0.010 | 2.800 |
| 971 | I16F_V93I_R97H | 0.000 | 9.000 | 0.000 | 4.900 | 0.000 | 5.060 |
| 972 | I16F_V93I_R97P | 0.000 | 5.240 | 0.000 | 3.500 | 0.000 | 4.120 |
| 973 | I16F_V93I_R97W | 0.000 | 9.090 | 0.000 | 6.220 | 0.000 | 5.200 |
| 974 | I16F_V93I_R119Q | 0.030 | 4.250 | 0.030 | 6.130 | 0.070 | 4.110 |
| 975 | I16F_V93K | 0.000 | 9.370 | 0.000 | 8.270 | 0.000 | 7.450 |
| 976 | I16F_V93K_R97A | 0.000 | 4.320 | 0.000 | 5.360 | 0.010 | 2.940 |
| 977 | I16F_V93K_R97F | 0.000 | 7.840 | 0.000 | 7.190 | 0.000 | 5.030 |
| 978 | I16F_V93K_R97H | 0.000 | 6.010 | 0.000 | 5.190 | 0.270 | 3.260 |
| 979 | I16F_V93K_R97W | 0.000 | 5.530 | 0.000 | 6.820 | 0.920 | 3.380 |
| 980 | I16F_V93Q | 0.000 | 9.220 | 0.000 | 9.060 | 0.000 | 8.500 |
| 981 | I16F_V93Q_R97A | 0.000 | 6.680 | 0.000 | 8.000 | 0.000 | 7.020 |
| 982 | I16F_V93Q_R97F | 0.000 | 6.110 | 0.000 | 5.390 | 0.000 | 5.120 |
| 983 | I16F_V93Q_R97H | 0.000 | 7.020 | 0.000 | 6.670 | 0.000 | 4.890 |
| 984 | I16F_V93Q_R97W | 0.000 | 6.630 | 0.000 | 6.150 | 0.290 | 3.160 |
| 985 | I16F_V93R | 0.000 | 9.550 | 0.000 | 9.270 | 0.000 | 7.750 |
| 986 | I16F_V93R_R97A | 0.000 | 8.650 | 0.000 | 8.350 | 0.000 | 6.370 |
| 987 | I16F_V93R_R97A_R119M | 0.000 | 6.260 | 0.000 | 5.390 | 0.000 | 4.720 |
| 988 | I16F_V93R_R97A_R119Q | 0.000 | 5.840 | 0.000 | 5.870 | 0.000 | 5.660 |
| 989 | I16F_V93R_R97A_R119Y | 0.000 | 3.000 | 1.000 | −0.040 | 1.000 | −2.680 |
| 990 | I16F_V93R_R97F | 0.000 | 7.870 | 0.000 | 7.540 | 0.000 | 5.350 |
| 991 | I16F_V93R_R97F_R119Q | 0.000 | 4.370 | 0.000 | 4.240 | 0.000 | 3.300 |
| 992 | I16F_V93R_R97H | 0.000 | 7.850 | 0.000 | 8.750 | 0.000 | 6.160 |
| 993 | I16F_V93R_R119M | 0.020 | 4.720 | 0.010 | 4.210 | 1.000 | 0.840 |
| 994 | I16F_V93R_R119Q | 0.000 | 6.350 | 0.050 | 5.780 | 0.000 | 5.330 |
| 995 | I16H_P90F_V93I | 0.000 | 2.180 | 1.000 | 0.890 | 0.180 | 1.100 |
| 996 | I16H_P90W_V93I | 0.000 | 4.860 | 0.010 | 4.510 | 0.120 | 3.180 |
| 997 | I16H_R97A | 0.000 | 5.580 | 1.000 | 0.830 | 1.000 | −2.190 |
| 998 | I16H_R97H | 0.000 | 3.320 | 1.000 | −0.080 | 1.000 | −2.680 |
| 999 | I16H_V93I_R97A | 0.000 | 4.870 | 1.000 | −0.880 | 1.000 | −2.220 |
| 1000 | I16H_V93I_R97H | 0.000 | 3.420 | 1.000 | −1.730 | 1.000 | −2.870 |
| 1001 | I16H_V93Q | 0.000 | 4.210 | 0.300 | 3.070 | 1.000 | −0.140 |
| 1002 | I16L | 0.000 | 5.435 | 0.000 | 4.617 | 0.000 | 4.445 |
| 1003 | I16L_P36A_P90F | 0.000 | 4.650 | 0.000 | 5.460 | 0.000 | 4.240 |
| 1004 | I16L_P36A_P90F_V93I | 0.000 | 6.420 | 0.000 | 5.800 | 0.000 | 6.630 |
| 1005 | I16L_P36A_P90F_V93I_R97A | 0.000 | 5.480 | 0.000 | 3.170 | 0.840 | 2.930 |
| 1006 | I16L_P36A_P90F_V93I_R97F | 0.000 | 3.690 | 0.130 | 3.010 | 1.000 | 0.620 |
| 1007 | I16L_P36A_P90F_V93I_R97H | 0.560 | 3.080 | 0.000 | 3.530 | 1.000 | 1.780 |
| 1008 | I16L_P36A_P90I_R97A | 0.000 | 2.730 | 1.000 | −1.400 | 1.000 | −0.600 |
| 1009 | I16L_P36A_P90I_V93I | 0.000 | 2.910 | 1.000 | 0.830 | 0.210 | 2.630 |
| 1010 | I16L_P36A_P90I_V93I_R97A | 1.000 | 1.600 | 1.000 | 1.250 | 0.000 | 2.740 |
| 1011 | I16L_P36A_P90W | 0.010 | 2.810 | 0.440 | 2.300 | 0.000 | 3.490 |
| 1012 | I16L_P36A_P90W_V93I | 0.000 | 6.530 | 0.000 | 6.840 | 0.000 | 5.510 |
| 1013 | I16L_P36A_P90Y_V93I | 0.000 | 7.240 | 0.000 | 5.970 | 0.000 | 6.370 |
| 1014 | I16L_P36A_R97A | 0.000 | 3.680 | 1.000 | −1.880 | 1.000 | −1.470 |
| 1015 | I16L_P36A_R97F | 0.000 | 5.420 | 1.000 | 0.410 | 1.000 | −1.100 |
| 1016 | I16L_P36A_R97W | 0.000 | 5.130 | 0.060 | 0.800 | 1.000 | −0.360 |
| 1017 | I16L_P36A_V93E | 0.000 | 3.560 | 0.010 | 1.770 | 1.000 | −0.140 |
| 1018 | I16L_P36A_V93I_R97A | 0.000 | 3.890 | 1.000 | −0.150 | 1.000 | 0.380 |
| 1019 | I16L_P36A_V93I_R97F | 0.000 | 4.630 | 1.000 | −0.660 | 1.000 | −0.230 |
| 1020 | I16L_P36A_V93I_R97W | 0.000 | 5.130 | 1.000 | −0.510 | 1.000 | −1.110 |
| 1021 | I16L_P36A_V93K | 0.000 | 6.750 | 0.000 | 5.840 | 0.040 | 3.680 |
| 1022 | I16L_P36A_V93K_R97F | 1.000 | 2.500 | 0.000 | 3.820 | 1.000 | 1.510 |
| 1023 | I16L_P36A_V93Q | 0.000 | 6.790 | 0.000 | 3.480 | 1.000 | 2.310 |
| 1024 | I16L_P36A_V93Q_R97A | 0.000 | 5.830 | 0.000 | 4.740 | 0.230 | 2.580 |
| 1025 | I16L_P36A_V93Q_R97F | 1.000 | 1.590 | 0.000 | 3.700 | 1.000 | 0.760 |
| 1026 | I16L_P36A_V93Q_R97H | 1.000 | 2.310 | 0.030 | 3.080 | 1.000 | 0.990 |
| 1027 | I16L_P36A_V93R | 0.000 | 4.750 | 0.020 | 2.530 | 0.000 | 3.240 |
| 1028 | I16L_P36A_V93R_R97A | 0.000 | 5.460 | 0.000 | 4.740 | 1.000 | 2.390 |
| 1029 | I16L_P36A_V93R_R97A_R119Q | 0.000 | 2.110 | 1.000 | 0.950 | 1.000 | 1.870 |
| 1030 | I16L_P36A_V93R_R97F | 0.000 | 3.940 | 0.000 | 3.480 | 1.000 | 1.760 |
| 1031 | I16L_P36A_V93R_R97H | 0.000 | 6.000 | 0.000 | 6.270 | 1.000 | 1.730 |
| 1032 | I16L_P36H | 0.000 | 8.690 | 0.000 | 7.360 | 0.000 | 5.350 |
| 1033 | I16L_P36H_P90F | 0.000 | 6.170 | 0.000 | 5.730 | 0.000 | 4.950 |
| 1034 | I16L_P36H_P90I_V93I_R97F | 0.000 | 2.690 | 0.000 | 2.320 | 1.000 | 1.480 |
| 1035 | I16L_P36H_P90W | 0.000 | 4.100 | 0.000 | 3.480 | 0.000 | 3.150 |
| 1036 | I16L_P36H_P90W_V93I | 0.000 | 3.840 | 0.070 | 2.300 | 0.090 | 1.020 |
| 1037 | I16L_P36H_P90Y_V93I_R97A | 0.000 | 4.660 | 0.000 | 4.420 | 0.000 | 3.180 |
| 1038 | I16L_P36H_R97F | 0.000 | 5.550 | 0.000 | 5.190 | 1.000 | 1.450 |
| 1039 | I16L_P36H_R97H | 0.010 | 4.420 | 0.000 | 5.610 | 1.000 | 0.450 |
| 1040 | I16L_P36H_V93I | 0.000 | 7.880 | 0.000 | 7.940 | 0.000 | 5.480 |
| 1041 | I16L_P36H_V93I_R97A | 0.000 | 4.670 | 0.000 | 4.430 | 1.000 | 1.520 |
| 1042 | I16L_P36H_V93I_R97F | 0.000 | 4.080 | 0.000 | 4.290 | 0.340 | 3.090 |
| 1043 | I16L_P36H_V93I_R97H | 0.000 | 4.870 | 0.000 | 3.950 | 0.010 | 3.200 |
| 1044 | I16L_P36H_V93I_R97W | 0.000 | 4.910 | 0.000 | 5.090 | 0.000 | 4.180 |
| 1045 | I16L_P36H_V93Q | 0.000 | 7.450 | 0.000 | 5.250 | 1.000 | 2.870 |
| 1046 | I16L_P36H_V93Q_R97F | 0.000 | 2.670 | 0.000 | 2.250 | 1.000 | 0.110 |
| 1047 | I16L_P36H_V93Q_R97W | 1.000 | −0.430 | 0.000 | 2.490 | 1.000 | −3.220 |
| 1048 | I16L_P36H_V93R | 0.000 | 4.940 | 0.000 | 4.300 | 0.000 | 3.290 |
| 1049 | I16L_P36H_V93R_R97A | 0.000 | 4.450 | 0.080 | 2.200 | 1.000 | −0.290 |
| 1050 | I16L_P36R | 0.000 | 8.320 | 0.000 | 7.810 | 0.000 | 5.380 |
| 1051 | I16L_P36R_R97A | 0.000 | 4.800 | 0.000 | 5.540 | 0.050 | 3.780 |
| 1052 | I16L_P36R_R97W | 1.000 | 2.270 | 0.000 | 3.100 | 0.000 | 2.640 |
| 1053 | I16L_P36R_V93I | 0.000 | 5.520 | 1.000 | 0.660 | 1.000 | 0.740 |
| 1054 | I16L_P36R_V93I_R97H | 0.000 | 2.720 | 1.000 | −0.260 | 1.000 | −0.570 |
| 1055 | I16L_P36R_V93K | 0.000 | 3.170 | 0.190 | 1.560 | 0.630 | 1.200 |
| 1056 | I16L_P36S_P90F_V93I | 0.000 | 7.560 | 0.000 | 4.870 | 0.000 | 4.410 |
| 1057 | I16L_P36S_P90F_V93I_R97A | 0.000 | 2.370 | 0.000 | 1.950 | 0.060 | 1.220 |
| 1058 | I16L_P36S_V93E | 0.000 | 3.260 | 1.000 | 1.660 | 1.000 | 0.100 |
| 1059 | I16L_P36S_V93I_R97F | 0.000 | 3.930 | 1.000 | −1.820 | 1.000 | −3.140 |
| 1060 | I16L_P36S_V93I_R97H | 0.000 | 2.000 | 0.750 | 1.290 | 0.570 | 1.110 |
| 1061 | I16L_P36S_V93K_R97A | 0.000 | 2.810 | 0.140 | 1.840 | 1.000 | −0.550 |
| 1062 | I16L_P36S_V93Q_R97A | 1.000 | 2.000 | 0.000 | 4.530 | 1.000 | −0.920 |
| 1063 | I16L_P36S_V93R | 0.000 | 4.120 | 0.030 | 3.870 | 1.000 | 0.640 |
| 1064 | I16L_P36S_V93R_R97W | 0.010 | 2.560 | 0.040 | 2.090 | 1.000 | −0.450 |
| 1065 | I16L_P36W | 0.000 | 6.790 | 0.000 | 5.330 | 0.000 | 6.800 |
| 1066 | I16L_P36W_P90F | 0.000 | 5.010 | 0.000 | 5.020 | 0.000 | 5.300 |
| 1067 | I16L_P36W_P90F_R97A | 0.000 | 4.020 | 0.000 | 3.060 | 1.000 | 2.560 |
| 1068 | I16L_P36W_P90F_V93I | 0.000 | 7.980 | 0.000 | 7.630 | 0.000 | 7.280 |
| 1069 | I16L_P36W_P90F_V93I_R97A | 0.000 | 5.360 | 0.000 | 4.960 | 0.010 | 3.430 |
| 1070 | I16L_P36W_P90F_V93I_R97F | 0.010 | 2.910 | 0.230 | 2.620 | 1.000 | 0.020 |
| 1071 | I16L_P36W_P90F_V93I_R97H | 0.000 | 4.120 | 0.000 | 4.000 | 0.500 | 2.340 |
| 1072 | I16L_P36W_P90F_V93I_R97W | 0.050 | 3.560 | 1.000 | 1.180 | 1.000 | −0.780 |
| 1073 | I16L_P36W_P90F_V93R_R97A | 1.000 | 1.800 | 0.000 | 2.350 | 1.000 | −0.580 |
| 1074 | I16L_P36W_P90I | 0.000 | 3.540 | 1.000 | 0.770 | 1.000 | 1.850 |
| 1075 | I16L_P36W_P90I_V93I | 0.340 | 2.350 | 0.260 | 2.510 | 0.040 | 3.080 |
| 1076 | I16L_P36W_P90I_V93I_R97A | 0.000 | 2.440 | 0.760 | 2.300 | 1.000 | 0.000 |
| 1077 | I16L_P36W_P90W | 0.000 | 3.570 | 0.000 | 3.020 | 0.230 | 2.370 |
| 1078 | I16L_P36W_P90W_R97A | 0.000 | 3.230 | 0.000 | 3.980 | 1.000 | 1.230 |
| 1079 | I16L_P36W_P90W_V93I | 0.000 | 7.390 | 0.000 | 4.340 | 0.000 | 3.540 |
| 1080 | I16L_P36W_P90W_V93I_R97A | 0.000 | 2.950 | 0.000 | 3.750 | 1.000 | 2.260 |
| 1081 | I16L_P36W_P90Y_V93R_R97A | 0.000 | 2.780 | 1.000 | 1.030 | 0.000 | 1.950 |
| 1082 | I16L_P36W_R97A | 0.000 | 5.200 | 0.000 | 4.650 | 0.530 | 3.340 |
| 1083 | I16L_P36W_R97W | 0.030 | 2.530 | 1.000 | 1.540 | 1.000 | 0.340 |
| 1084 | I16L_P36W_V93I | 0.000 | 8.440 | 0.000 | 5.890 | 0.000 | 7.750 |
| 1085 | I16L_P36W_V93I_R97A | 1.000 | 1.720 | 0.000 | 3.850 | 1.000 | 1.060 |
| 1086 | I16L_P36W_V93I_R97F | 0.000 | 3.370 | 0.020 | 3.110 | 1.000 | 1.750 |
| 1087 | I16L_P36W_V93I_R97H | 0.010 | 4.760 | 0.000 | 3.940 | 1.000 | 1.730 |
| 1088 | I16L_P36W_V93I_R97W | 0.030 | 2.450 | 1.000 | 2.040 | 1.000 | −0.220 |
| 1089 | I16L_P36W_V93Q | 0.000 | 3.780 | 0.000 | 4.040 | 1.000 | 0.350 |
| 1090 | I16L_P36W_V93R_R97A | 0.000 | 4.040 | 1.000 | 1.370 | 1.000 | 1.400 |
| 1091 | I16L_P90F | 0.000 | 9.580 | 0.000 | 8.420 | 0.000 | 9.110 |
| 1092 | I16L_P90F_R97A | 0.000 | 6.490 | 0.000 | 5.990 | 0.010 | 3.520 |
| 1093 | I16L_P90F_R97F | 0.000 | 6.520 | 0.000 | 6.310 | 0.000 | 5.710 |
| 1094 | I16L_P90F_R97H | 0.000 | 4.670 | 0.170 | 2.820 | 1.000 | 1.070 |
| 1095 | I16L_P90F_R97W | 0.030 | 3.810 | 0.000 | 3.760 | 0.000 | 2.020 |
| 1096 | I16L_P90F_R119Q | 0.000 | 2.070 | 0.440 | 1.520 | 0.010 | 1.430 |
| 1097 | I16L_P90F_V93I | 0.000 | 9.580 | 0.000 | 8.940 | 0.000 | 8.780 |
| 1098 | I16L_P90F_V93I_R97A | 0.000 | 6.580 | 0.000 | 6.850 | 0.000 | 5.930 |
| 1099 | I16L_P90F_V93I_R97F | 0.000 | 5.490 | 0.000 | 6.340 | 0.000 | 4.240 |
| 1100 | I16L_P90F_V93I_R97H | 0.000 | 6.110 | 0.000 | 6.370 | 0.000 | 4.150 |
| 1101 | I16L_P90F_V93I_R97W | 0.000 | 5.870 | 0.000 | 5.670 | 0.010 | 2.770 |
| 1102 | I16L_P90F_V93Q | 0.000 | 5.780 | 0.000 | 6.320 | 0.000 | 4.980 |
| 1103 | I16L_P90F_V93Q_R97A | 0.000 | 6.550 | 0.000 | 4.560 | 0.000 | 3.190 |
| 1104 | I16L_P90F_V93Q_R97F | 0.000 | 3.490 | 1.000 | 1.540 | 1.000 | −1.890 |
| 1105 | I16L_P90F_V93R | 0.000 | 2.800 | 1.000 | 2.180 | 1.000 | 1.880 |
| 1106 | I16L_P90F_V93R_R97F | 0.000 | 2.860 | 1.000 | −0.050 | 1.000 | 0.740 |
| 1107 | I16L_P90I | 0.000 | 5.690 | 0.140 | 1.790 | 1.000 | 1.680 |
| 1108 | I16L_P90I_R97A | 0.000 | 5.280 | 0.000 | 4.040 | 0.000 | 3.750 |
| 1109 | I16L_P90I_R97F | 0.760 | 3.050 | 0.030 | 3.990 | 1.000 | 1.770 |
| 1110 | I16L_P90I_R97H | 0.200 | 4.250 | 0.000 | 5.130 | 1.000 | 2.340 |
| 1111 | I16L_P90I_R97W | 0.000 | 5.030 | 1.000 | 1.880 | 1.000 | 1.670 |
| 1112 | I16L_P90I_V93I | 0.000 | 7.330 | 0.000 | 2.790 | 0.000 | 3.160 |
| 1113 | I16L_P90I_V93I_R97A | 0.000 | 5.290 | 0.010 | 4.220 | 0.650 | 2.360 |
| 1114 | I16L_P90I_V93I_R97F | 0.000 | 4.390 | 0.000 | 5.430 | 0.000 | 4.490 |
| 1115 | I16L_P90I_V93I_R97H | 0.000 | 4.720 | 0.000 | 5.690 | 0.310 | 2.540 |
| 1116 | I16L_P90I_V93I_R97W | 0.000 | 4.200 | 0.000 | 5.050 | 1.000 | 2.110 |
| 1117 | I16L_P90I_V93K | 0.080 | 4.800 | 0.000 | 4.330 | 1.000 | 0.380 |
| 1118 | I16L_P90I_V93K_R97F | 0.000 | 2.780 | 0.000 | 2.210 | 1.000 | 0.460 |
| 1119 | I16L_P90I_V93K_R97W | 0.000 | 3.660 | 1.000 | 1.860 | 1.000 | −1.660 |
| 1120 | I16L_P90I_V93Q | 0.000 | 6.000 | 0.010 | 3.330 | 1.000 | 2.040 |
| 1121 | I16L_P90I_V93Q_R97A | 1.000 | 2.170 | 0.000 | 5.060 | 1.000 | 0.290 |
| 1122 | I16L_P90I_V93Q_R97F | 0.000 | 3.030 | 0.000 | 2.960 | 1.000 | 0.960 |
| 1123 | I16L_P90I_V93R | 0.000 | 6.720 | 0.000 | 7.760 | 0.000 | 5.160 |
| 1124 | I16L_P90I_V93R_R97A | 0.000 | 5.090 | 0.000 | 3.840 | 1.000 | 1.350 |
| 1125 | I16L_P90I_V93R_R97F | 0.000 | 2.470 | 0.000 | 2.350 | 0.000 | 2.150 |
| 1126 | I16L_P90S | 0.000 | 3.020 | 1.000 | 1.010 | 0.160 | 1.650 |
| 1127 | I16L_P90S_V93I_R97H | 0.010 | 3.320 | 1.000 | −1.370 | 1.000 | −0.970 |
| 1128 | I16L_P90S_V93I_R97W | 0.030 | 4.610 | 1.000 | −0.230 | 1.000 | 1.030 |
| 1129 | I16L_P90S_V93Q | 0.000 | 4.080 | 0.000 | 3.720 | 0.000 | 3.570 |
| 1130 | I16L_P90S_V93R | 0.000 | 2.520 | 0.050 | 2.020 | 0.980 | 1.320 |
| 1131 | I16L_P90W | 0.000 | 8.600 | 0.000 | 8.140 | 0.000 | 7.970 |
| 1132 | I16L_P90W_R97A | 0.000 | 7.720 | 0.000 | 6.950 | 0.000 | 6.470 |
| 1133 | I16L_P90W_R97F | 0.000 | 5.280 | 0.000 | 4.690 | 0.000 | 4.120 |
| 1134 | I16L_P90W_R97H | 0.000 | 6.020 | 0.000 | 5.080 | 0.000 | 4.130 |
| 1135 | I16L_P90W_V93E_R97H | 0.000 | 2.720 | 1.000 | 1.120 | 0.060 | 2.000 |
| 1136 | I16L_P90W_V93I | 0.000 | 9.940 | 0.000 | 8.160 | 0.000 | 8.980 |
| 1137 | I16L_P90W_V93I_R97A | 0.000 | 4.950 | 0.000 | 5.480 | 0.000 | 4.110 |
| 1138 | I16L_P90W_V93I_R97F | 0.000 | 4.670 | 0.200 | 4.360 | 1.000 | 2.400 |
| 1139 | I16L_P90W_V93I_R97H | 0.000 | 6.480 | 0.000 | 5.970 | 0.000 | 4.720 |
| 1140 | I16L_P90W_V93I_R97W | 0.000 | 4.280 | 0.020 | 4.190 | 0.080 | 2.590 |
| 1141 | I16L_P90W_V93Q | 0.000 | 6.220 | 0.000 | 6.640 | 0.000 | 3.480 |
| 1142 | I16L_P90W_V93Q_R97A | 0.000 | 4.330 | 0.670 | 3.150 | 1.000 | 1.580 |
| 1143 | I16L_P90W_V93Q_R97F | 0.000 | 3.230 | 0.000 | 2.920 | 1.000 | 2.000 |
| 1144 | I16L_P90W_V93Q_R97H | 0.000 | 3.140 | 1.000 | 2.980 | 1.000 | 0.610 |
| 1145 | I16L_P90W_V93R_R97A | 0.000 | 3.820 | 0.070 | 4.670 | 0.000 | 2.610 |
| 1146 | I16L_P90Y | 0.000 | 8.180 | 0.000 | 5.930 | 0.000 | 6.480 |
| 1147 | I16L_P90Y_R97A | 0.000 | 4.320 | 0.000 | 5.860 | 1.000 | 1.880 |
| 1148 | I16L_P90Y_R97F | 0.010 | 2.400 | 0.000 | 3.710 | 1.000 | 1.160 |
| 1149 | I16L_P90Y_R97H | 0.000 | 4.570 | 0.090 | 2.970 | 1.000 | 2.020 |
| 1150 | I16L_P90Y_R97W | 0.000 | 2.900 | 1.000 | 2.040 | 0.960 | 1.840 |
| 1151 | I16L_P90Y_V93I | 0.000 | 8.670 | 0.000 | 9.200 | 0.000 | 8.420 |
| 1152 | I16L_P90Y_V93I_R97A | 0.000 | 4.930 | 0.000 | 4.000 | 0.000 | 3.710 |
| 1153 | I16L_P90Y_V93I_R97F | 0.000 | 5.620 | 0.000 | 5.690 | 0.040 | 2.750 |
| 1154 | I16L_P90Y_V93I_R97H | 0.000 | 5.540 | 0.000 | 4.790 | 0.000 | 3.910 |
| 1155 | I16L_P90Y_V93I_R97W | 1.000 | 2.730 | 0.000 | 3.550 | 1.000 | 1.750 |
| 1156 | I16L_P90Y_V93K_R97A | 0.000 | 2.910 | 1.000 | 1.220 | 1.000 | 0.280 |
| 1157 | I16L_P90Y_V93K_R97F | 1.000 | −0.010 | 0.000 | 2.050 | 1.000 | 0.680 |
| 1158 | I16L_P90Y_V93Q | 0.000 | 6.220 | 0.000 | 5.610 | 0.010 | 3.760 |
| 1159 | I16L_P90Y_V93R_R97A | 0.000 | 5.550 | 0.000 | 4.200 | 0.000 | 4.440 |
| 1160 | I16L_R97A | 0.000 | 5.070 | 0.000 | 4.310 | 0.000 | 4.130 |
| 1161 | I16L_R97F | 0.000 | 5.180 | 0.000 | 3.840 | 0.000 | 3.430 |
| 1162 | I16L_R97F_R119Q | 0.000 | 2.180 | 1.000 | −1.010 | 1.000 | −0.980 |
| 1163 | I16L_R97H | 0.000 | 3.740 | 1.000 | −0.310 | 1.000 | −0.020 |
| 1164 | I16L_R97W | 0.000 | 4.450 | 1.000 | 0.730 | 1.000 | 0.030 |
| 1165 | I16L_V93E | 0.000 | 4.300 | 1.000 | 1.440 | 1.000 | 1.660 |
| 1166 | I16L_V93E_R97F | 0.000 | 4.070 | 0.000 | 3.370 | 1.000 | −0.440 |
| 1167 | I16L_V93E_R97H | 0.000 | 2.840 | 1.000 | 1.210 | 1.000 | −3.060 |
| 1168 | I16L_V93I | 0.000 | 2.960 | 0.040 | 1.430 | 0.000 | 1.530 |
| 1169 | I16L_V93I_R97A | 0.000 | 4.270 | 1.000 | 1.040 | 0.230 | 1.190 |
| 1170 | I16L_V93I_R97A_R119Q | 0.030 | 2.990 | 0.070 | 2.420 | 1.000 | 2.020 |
| 1171 | I16L_V93I_R97F | 0.000 | 3.980 | 0.000 | 1.430 | 1.000 | 0.650 |
| 1172 | I16L_V93I_R97F_R119Q | 0.000 | 3.190 | 1.000 | 0.930 | 1.000 | 0.540 |
| 1173 | I16L_V93I_R97W | 0.000 | 5.560 | 0.000 | 4.450 | 0.000 | 4.170 |
| 1174 | I16L_V93I_R119Q | 0.000 | 5.460 | 0.150 | 3.900 | 0.340 | 3.130 |
| 1175 | I16L_V93K | 0.000 | 7.940 | 0.000 | 6.510 | 0.000 | 6.530 |
| 1176 | I16L_V93K_R97A | 0.000 | 5.090 | 0.000 | 4.750 | 0.000 | 4.660 |
| 1177 | I16L_V93K_R97F | 0.000 | 4.280 | 0.010 | 2.480 | 1.000 | 2.690 |
| 1178 | I16L_V93K_R97H | 0.000 | 4.170 | 0.000 | 4.990 | 1.000 | 2.480 |
| 1179 | I16L_V93K_R97P | 0.000 | 2.370 | 1.000 | −0.870 | 0.290 | 1.280 |
| 1180 | I16L_V93K_R97W | 0.000 | 5.310 | 0.200 | 3.860 | 0.140 | 3.430 |
| 1181 | I16L_V93K_R119Q | 0.000 | 3.550 | 1.000 | 1.320 | 1.000 | 2.310 |
| 1182 | I16L_V93Q | 0.000 | 7.940 | 0.000 | 3.890 | 0.200 | 2.360 |
| 1183 | I16L_V93Q_R97A | 0.000 | 5.820 | 0.000 | 4.510 | 1.000 | 2.200 |
| 1184 | I16L_V93Q_R97F | 0.000 | 4.380 | 0.000 | 3.970 | 0.000 | 4.080 |
| 1185 | I16L_V93Q_R97H | 0.420 | 3.380 | 0.000 | 4.570 | 1.000 | 2.780 |
| 1186 | I16L_V93Q_R97W | 0.000 | 6.280 | 0.000 | 5.890 | 0.000 | 5.420 |
| 1187 | I16L_V93R | 0.000 | 9.220 | 0.000 | 8.690 | 0.000 | 7.790 |
| 1188 | I16L_V93R_R97A | 0.000 | 5.090 | 0.000 | 5.460 | 0.050 | 3.290 |
| 1189 | I16L_V93R_R97A_R119M | 1.000 | 1.290 | 0.000 | 3.950 | 1.000 | 1.860 |
| 1190 | I16L_V93R_R97A_R119Q | 0.010 | 2.610 | 0.000 | 3.110 | 1.000 | 0.440 |
| 1191 | I16L_V93R_R97F | 0.000 | 6.120 | 0.000 | 5.640 | 0.280 | 3.600 |
| 1192 | I16L_V93R_R97H | 0.000 | 4.830 | 0.000 | 4.030 | 1.000 | 2.180 |
| 1193 | I16L_V93R_R97W | 0.000 | 4.610 | 0.000 | 4.200 | 0.010 | 4.150 |
| 1194 | I16L_V93R_R97W_R119Q | 0.050 | 3.490 | 0.080 | 3.910 | 1.000 | −0.400 |
| 1195 | I16L_V93R_R119Q | 0.000 | 4.950 | 0.000 | 3.940 | 0.010 | 3.580 |
| 1196 | I16M_P36A_P90F | 0.000 | 5.310 | 0.000 | 4.700 | 0.000 | 4.990 |
| 1197 | I16M_P36A_P90F_R97A | 0.000 | 3.310 | 0.000 | 2.620 | 0.000 | 3.030 |
| 1198 | I16M_P36A_P90F_V93I | 0.000 | 6.310 | 0.000 | 4.780 | 0.000 | 5.040 |
| 1199 | I16M_P36A_P90F_V93I_R97A | 0.000 | 3.310 | 0.000 | 3.590 | 1.000 | 2.160 |
| 1200 | I16M_P36A_P90F_V93I_R97F | 0.330 | 2.050 | 0.000 | 3.170 | 1.000 | −0.200 |
| 1201 | I16M_P36A_P90F_V93I_R97H | 0.000 | 3.690 | 1.000 | 1.620 | 1.000 | 0.520 |
| 1202 | I16M_P36A_P90I | 0.000 | 3.070 | 1.000 | 0.650 | 0.000 | 2.850 |
| 1203 | I16M_P36A_P90I_V93I | 0.000 | 4.130 | 1.000 | 0.470 | 1.000 | 0.560 |
| 1204 | I16M_P36A_P90I_V93Q | 1.000 | −2.320 | 0.000 | 2.170 | 1.000 | −2.720 |
| 1205 | I16M_P36A_P90W | 0.000 | 2.360 | 0.080 | 2.710 | 1.000 | 2.180 |
| 1206 | I16M_P36A_P90W_V93I_R97A | 1.000 | 0.850 | 0.020 | 2.460 | 1.000 | −3.690 |
| 1207 | I16M_P36A_P90Y | 0.000 | 3.870 | 0.000 | 2.890 | 0.000 | 3.590 |
| 1208 | I16M_P36A_P90Y_V93I | 0.000 | 4.170 | 0.000 | 4.670 | 0.000 | 3.050 |
| 1209 | I16M_P36A_P90Y_V93I_R97W | 0.000 | 2.970 | 0.020 | 2.120 | 0.000 | 2.750 |
| 1210 | I16M_P36A_R97F | 0.000 | 4.510 | 0.000 | 3.100 | 0.000 | 3.740 |
| 1211 | I16M_P36A_R97W | 0.000 | 3.920 | 1.000 | 0.320 | 1.000 | −0.350 |
| 1212 | I16M_P36A_V93I_R97F | 0.000 | 3.420 | 1.000 | −0.200 | 1.000 | −0.020 |
| 1213 | I16M_P36A_V93K | 0.000 | 4.680 | 0.080 | 3.770 | 0.060 | 3.050 |
| 1214 | I16M_P36A_V93K_R97W | 0.000 | 2.240 | 1.000 | 1.110 | 1.000 | 0.380 |
| 1215 | I16M_P36A_V93Q | 0.000 | 3.990 | 0.170 | 3.550 | 1.000 | 1.450 |
| 1216 | I16M_P36A_V93Q_R97A | 0.000 | 3.330 | 0.000 | 2.630 | 0.000 | 2.540 |
| 1217 | I16M_P36A_V93Q_R97W | 0.000 | 2.920 | 0.000 | 2.930 | 0.000 | 2.340 |
| 1218 | I16M_P36A_V93R | 0.000 | 5.470 | 0.000 | 5.020 | 0.000 | 3.360 |
| 1219 | I16M_P36A_V93R_R97A | 0.000 | 4.030 | 1.000 | 3.190 | 1.000 | 1.570 |
| 1220 | I16M_P36A_V93R_R97W | 1.000 | 1.740 | 0.000 | 3.220 | 1.000 | −0.880 |
| 1221 | I16M_P36H | 0.000 | 4.950 | 0.000 | 6.420 | 0.050 | 3.040 |
| 1222 | I16M_P36H_P90F_V93I | 0.030 | 2.660 | 1.000 | 1.400 | 1.000 | 2.030 |
| 1223 | I16M_P36H_R97A | 1.000 | 1.230 | 0.000 | 2.720 | 1.000 | 0.170 |
| 1224 | I16M_P36H_R97F | 0.000 | 3.910 | 0.000 | 3.360 | 1.000 | 1.260 |
| 1225 | I16M_P36H_R97W | 0.030 | 2.900 | 0.000 | 3.590 | 1.000 | 0.970 |
| 1226 | I16M_P36H_V93I | 0.000 | 7.280 | 0.000 | 6.380 | 0.000 | 3.620 |
| 1227 | I16M_P36H_V93I_R97A | 0.000 | 3.980 | 0.000 | 3.340 | 1.000 | −1.090 |
| 1228 | I16M_P36H_V93I_R97H | 0.000 | 3.840 | 0.000 | 4.280 | 1.000 | 0.160 |
| 1229 | I16M_P36H_V93R | 1.000 | 1.260 | 0.020 | 2.790 | 1.000 | −0.900 |
| 1230 | I16M_P36R | 0.000 | 6.670 | 0.000 | 6.220 | 0.000 | 4.350 |
| 1231 | I16M_P36R_R97F | 0.000 | 4.030 | 0.000 | 2.680 | 1.000 | 1.890 |
| 1232 | I16M_P36R_V93I | 0.000 | 4.010 | 1.000 | 0.990 | 1.000 | 2.210 |
| 1233 | I16M_P36R_V93Q_R97A | 0.010 | 2.430 | 0.000 | 2.320 | 1.000 | 1.090 |
| 1234 | I16M_P36S_P90F | 0.000 | 2.350 | 0.010 | 1.940 | 0.000 | 2.370 |
| 1235 | I16M_P36S_P90F_V93I | 1.000 | 1.500 | 0.020 | 3.490 | 1.000 | 2.610 |
| 1236 | I16M_P36S_P90Y_V93I | 0.000 | 4.530 | 1.000 | −0.070 | 1.000 | 0.320 |
| 1237 | I16M_P36S_V93I_R97A | 0.000 | 2.600 | 0.260 | 1.340 | 0.910 | 1.430 |
| 1238 | I16M_P36S_V93Q_R97F | 0.000 | 2.640 | 1.000 | −0.040 | 0.840 | 2.580 |
| 1239 | I16M_P36W | 0.000 | 4.990 | 0.000 | 4.670 | 0.000 | 4.640 |
| 1240 | I16M_P36W_P90F | 0.000 | 5.560 | 0.000 | 5.150 | 0.000 | 4.650 |
| 1241 | I16M_P36W_P90F_R97H | 0.000 | 2.120 | 1.000 | 0.720 | 1.000 | 1.980 |
| 1242 | I16M_P36W_P90F_V93I | 0.000 | 5.920 | 0.000 | 6.150 | 0.000 | 5.400 |
| 1243 | I16M_P36W_P90F_V93I_R97A | 0.000 | 4.120 | 0.000 | 3.740 | 0.000 | 3.670 |
| 1244 | I16M_P36W_P90F_V93I_R97F | 0.000 | 2.300 | 1.000 | 1.900 | 1.000 | 1.800 |
| 1245 | I16M_P36W_P90F_V93R_R97A | 0.000 | 2.650 | 1.000 | −0.280 | 1.000 | 0.630 |
| 1246 | I16M_P36W_P90I | 0.000 | 3.240 | 1.000 | 0.180 | 1.000 | 1.380 |
| 1247 | I16M_P36W_P90I_V93I | 0.000 | 2.350 | 1.000 | −0.260 | 0.140 | 2.010 |
| 1248 | I16M_P36W_P90W | 0.000 | 3.780 | 0.000 | 3.570 | 0.000 | 4.600 |
| 1249 | I16M_P36W_P90W_R97A | 0.920 | 2.620 | 0.010 | 3.230 | 1.000 | 0.100 |
| 1250 | I16M_P36W_P90W_V93I | 0.000 | 7.050 | 0.000 | 5.720 | 0.000 | 5.060 |
| 1251 | I16M_P36W_P90W_V93I_R97A | 0.000 | 4.070 | 0.000 | 2.880 | 1.000 | 1.860 |
| 1252 | I16M_P36W_P90W_V93I_R97F | 0.000 | 2.870 | 1.000 | 0.630 | 1.000 | −0.460 |
| 1253 | I16M_P36W_P90W_V93I_R97H | 0.000 | 4.130 | 0.540 | 1.820 | 1.000 | 1.820 |
| 1254 | I16M_P36W_P90Y | 0.000 | 4.250 | 1.000 | 1.140 | 1.000 | 1.860 |
| 1255 | I16M_P36W_P90Y_V93I | 0.000 | 3.680 | 1.000 | 1.080 | 1.000 | 1.720 |
| 1256 | I16M_P36W_R97A | 0.030 | 2.850 | 1.000 | 1.470 | 1.000 | 0.790 |
| 1257 | I16M_P36W_V93I | 0.000 | 6.440 | 0.000 | 6.170 | 0.000 | 6.320 |
| 1258 | I16M_P36W_V93I_R97A | 0.000 | 3.840 | 0.000 | 4.030 | 0.000 | 3.020 |
| 1259 | I16M_P36W_V93I_R97F | 0.000 | 3.980 | 0.000 | 2.620 | 1.000 | 0.790 |
| 1260 | I16M_P36W_V93Q | 0.320 | 2.540 | 0.000 | 2.980 | 1.000 | 2.010 |
| 1261 | I16M_P90F | 0.000 | 7.350 | 0.000 | 7.140 | 0.000 | 6.400 |
| 1262 | I16M_P90F_R97A | 0.000 | 4.850 | 0.000 | 3.810 | 0.130 | 3.000 |
| 1263 | I16M_P90F_R97F | 0.000 | 3.720 | 0.000 | 4.180 | 1.000 | 1.970 |
| 1264 | I16M_P90F_R97H | 0.000 | 3.690 | 0.000 | 3.410 | 1.000 | 1.390 |
| 1265 | I16M_P90F_V93I | 0.000 | 8.420 | 0.000 | 7.240 | 0.000 | 7.580 |
| 1266 | I16M_P90F_V93I_R97A | 0.000 | 5.220 | 0.000 | 5.080 | 0.000 | 4.440 |
| 1267 | I16M_P90F_V93I_R97F | 0.000 | 4.740 | 0.000 | 4.450 | 0.330 | 3.350 |
| 1268 | I16M_P90F_V93I_R97H | 0.000 | 5.460 | 0.000 | 5.960 | 0.000 | 5.360 |
| 1269 | I16M_P90F_V93I_R97W | 0.530 | 5.390 | 0.000 | 2.850 | 1.000 | 2.220 |
| 1270 | I16M_P90F_V93K | 0.010 | 2.280 | 1.000 | 1.420 | 1.000 | 1.820 |
| 1271 | I16M_P90F_V93Q | 0.020 | 3.600 | 0.000 | 4.810 | 1.000 | 1.540 |
| 1272 | I16M_P90F_V93Q_R97A | 0.000 | 4.660 | 0.000 | 4.440 | 0.000 | 3.790 |
| 1273 | I16M_P90F_V93Q_R97H | 1.000 | 1.580 | 0.000 | 2.940 | 1.000 | 0.490 |
| 1274 | I16M_P90F_V93R | 0.000 | 2.950 | 0.310 | 3.300 | 1.000 | 0.220 |
| 1275 | I16M_P90I | 0.000 | 7.460 | 0.000 | 3.810 | 0.000 | 3.720 |
| 1276 | I16M_P90I_R97A | 1.000 | 1.790 | 0.030 | 3.540 | 1.000 | 0.320 |
| 1277 | I16M_P90I_R97F | 0.000 | 3.550 | 0.030 | 3.500 | 1.000 | 0.820 |
| 1278 | I16M_P90I_R97H | 0.920 | 2.620 | 0.000 | 4.020 | 1.000 | 2.240 |
| 1279 | I16M_P90I_R97W | 0.000 | 4.340 | 1.000 | 1.490 | 0.020 | 3.500 |
| 1280 | I16M_P90I_V93I | 0.000 | 6.420 | 0.030 | 1.980 | 0.000 | 2.630 |
| 1281 | I16M_P90I_V93I_R97F | 0.000 | 2.790 | 0.000 | 3.250 | 1.000 | 0.930 |
| 1282 | I16M_P90I_V93I_R97H | 0.020 | 3.710 | 0.010 | 4.210 | 1.000 | 2.070 |
| 1283 | I16M_P90I_V93I_R97W | 0.160 | 3.210 | 0.000 | 4.170 | 0.530 | 3.100 |
| 1284 | I16M_P90I_V93K | 0.100 | 3.310 | 0.000 | 3.950 | 1.000 | 1.040 |
| 1285 | I16M_P90I_V93K_R97A | 0.010 | 2.020 | 1.000 | 1.290 | 1.000 | 0.400 |
| 1286 | I16M_P90I_V93K_R97F | 1.000 | 1.580 | 0.020 | 2.320 | 1.000 | −1.030 |
| 1287 | I16M_P90I_V93Q | 0.000 | 5.660 | 0.000 | 5.130 | 1.000 | 1.020 |
| 1288 | I16M_P90I_V93R | 0.000 | 5.110 | 0.000 | 5.220 | 1.000 | 1.800 |
| 1289 | I16M_P90S_V93I_R97H | 0.000 | 3.950 | 1.000 | −0.280 | 1.000 | −0.010 |
| 1290 | I16M_P90W | 0.000 | 6.680 | 0.000 | 6.820 | 0.000 | 7.380 |
| 1291 | I16M_P90W_R97A | 0.000 | 3.500 | 0.480 | 2.130 | 1.000 | 1.450 |
| 1292 | I16M_P90W_R97F | 0.000 | 4.400 | 1.000 | 2.020 | 1.000 | 1.480 |
| 1293 | I16M_P90W_R97H | 0.000 | 4.080 | 0.000 | 4.300 | 1.000 | 2.260 |
| 1294 | I16M_P90W_R97W | 0.000 | 2.840 | 0.000 | 2.330 | 1.000 | 1.690 |
| 1295 | I16M_P90W_V93I | 0.000 | 7.750 | 0.000 | 7.360 | 0.000 | 6.570 |
| 1296 | I16M_P90W_V93I_R97A | 0.000 | 4.870 | 0.000 | 5.090 | 0.000 | 4.120 |
| 1297 | I16M_P90W_V93I_R97F | 0.000 | 5.140 | 0.000 | 4.610 | 0.000 | 5.820 |
| 1298 | I16M_P90W_V93I_R97H | 0.000 | 4.770 | 0.000 | 4.300 | 0.210 | 2.490 |
| 1299 | I16M_P90W_V93I_R97W | 0.000 | 4.920 | 0.000 | 3.490 | 0.010 | 3.030 |
| 1300 | I16M_P90W_V93Q | 0.000 | 3.320 | 0.000 | 4.770 | 1.000 | 1.710 |
| 1301 | I16M_P90W_V93Q_R97W | 0.000 | 2.530 | 1.000 | −0.660 | 1.000 | 0.150 |
| 1302 | I16M_P90W_V93R | 0.370 | 2.410 | 0.040 | 3.280 | 1.000 | 0.780 |
| 1303 | I16M_P90W_V93R_R97A | 0.000 | 3.560 | 0.050 | 2.240 | 1.000 | 2.250 |
| 1304 | I16M_P90Y | 0.000 | 7.390 | 0.000 | 6.220 | 0.000 | 6.240 |
| 1305 | I16M_P90Y_R97F | 0.000 | 3.630 | 0.000 | 3.200 | 1.000 | 1.400 |
| 1306 | I16M_P90Y_R97H | 0.000 | 4.850 | 0.000 | 2.470 | 1.000 | 0.550 |
| 1307 | I16M_P90Y_V93I | 0.000 | 7.220 | 0.000 | 7.290 | 0.000 | 7.860 |
| 1308 | I16M_P90Y_V93I_R97A | 0.000 | 5.250 | 0.000 | 4.970 | 1.000 | 2.190 |
| 1309 | I16M_P90Y_V93I_R97F | 0.000 | 4.780 | 0.000 | 4.800 | 0.000 | 3.680 |
| 1310 | I16M_P90Y_V93I_R97H | 0.000 | 5.160 | 0.000 | 5.140 | 0.000 | 4.880 |
| 1311 | I16M_P90Y_V93I_R97W | 1.000 | 3.510 | 0.010 | 2.560 | 1.000 | 0.680 |
| 1312 | I16M_R97A | 0.000 | 2.900 | 0.000 | 2.310 | 0.030 | 2.690 |
| 1313 | I16M_R97F | 0.040 | 2.840 | 1.000 | 0.410 | 1.000 | 1.040 |
| 1314 | I16M_R97H | 0.010 | 2.250 | 0.760 | 1.620 | 1.000 | 1.270 |
| 1315 | I16M_V93I | 0.000 | 4.050 | 0.000 | 2.470 | 0.000 | 2.750 |
| 1316 | I16M_V93I_R97A | 0.010 | 2.520 | 1.000 | 0.270 | 1.000 | 0.220 |
| 1317 | I16M_V93I_R97F | 0.000 | 3.070 | 1.000 | 0.300 | 1.000 | −0.540 |
| 1318 | I16M_V93I_R97H | 0.000 | 3.180 | 1.000 | 0.060 | 1.000 | 0.210 |
| 1319 | I16M_V93I_R97W | 0.000 | 3.980 | 0.000 | 3.430 | 0.000 | 3.380 |
| 1320 | I16M_V93I_R119Q | 0.000 | 3.130 | 0.000 | 3.750 | 1.000 | 2.580 |
| 1321 | I16M_V93K | 0.000 | 5.420 | 0.000 | 6.010 | 0.000 | 4.500 |
| 1322 | I16M_V93K_R97A | 1.000 | 1.960 | 0.010 | 3.550 | 1.000 | 0.530 |
| 1323 | I16M_V93K_R97F | 0.010 | 3.510 | 0.000 | 3.200 | 1.000 | 2.510 |
| 1324 | I16M_V93K_R97W | 0.000 | 3.270 | 0.000 | 2.990 | 1.000 | 1.970 |
| 1325 | I16M_V93Q | 0.000 | 5.750 | 0.000 | 3.750 | 1.000 | 1.520 |
| 1326 | I16M_V93Q_R97A | 0.010 | 3.400 | 0.000 | 4.100 | 0.000 | 3.600 |
| 1327 | I16M_V93Q_R97F | 1.000 | 2.790 | 0.000 | 4.180 | 1.000 | 1.790 |
| 1328 | I16M_V93Q_R97H | 0.000 | 4.300 | 1.000 | 1.800 | 1.000 | 1.610 |
| 1329 | I16M_V93Q_R97W | 0.000 | 3.650 | 0.000 | 4.210 | 1.000 | 1.640 |
| 1330 | I16M_V93R | 0.000 | 5.110 | 0.000 | 4.920 | 0.000 | 4.320 |
| 1331 | I16M_V93R_R97A | 0.000 | 4.210 | 0.000 | 4.180 | 0.000 | 3.880 |
| 1332 | I16M_V93R_R97H | 0.000 | 4.550 | 0.000 | 3.920 | 0.010 | 3.160 |
| 1333 | I16M_V93R_R97H_R119Q | 1.000 | 1.040 | 0.050 | 2.810 | 1.000 | −0.400 |
| 1334 | I16M_V93R_R97W | 0.000 | 3.050 | 0.000 | 2.530 | 0.000 | 2.270 |
| 1335 | I16M_V93R_R119M | 0.000 | 3.320 | 0.050 | 2.460 | 1.000 | 0.770 |
| 1336 | I16M_V93R_R119Q | 0.000 | 5.800 | 0.140 | 3.380 | 1.000 | −0.310 |
| 97 | I16W | 0.000 | 10.067 | 0.000 | 9.448 | 0.000 | 9.479 |
| 1337 | I16W_P36A | 0.000 | 9.060 | 0.000 | 8.830 | 0.000 | 8.050 |
| 1338 | I16W_P36A_P90F_R97A | 0.000 | 3.380 | 0.020 | 2.050 | 0.000 | 1.910 |
| 1339 | I16W_P36A_R97A | 0.010 | 4.560 | 1.000 | 2.130 | 1.000 | 0.380 |
| 1340 | I16W_P36A_R97F | 0.000 | 3.130 | 0.720 | 2.480 | 1.000 | 0.500 |
| 1341 | I16W_P36A_R97H | 0.000 | 4.720 | 0.000 | 4.400 | 1.000 | 1.530 |
| 1342 | I16W_P36A_V93I | 0.000 | 8.030 | 0.000 | 5.420 | 0.000 | 5.550 |
| 1343 | I16W_P36A_V93I_R97A | 0.440 | 2.910 | 0.000 | 3.760 | 1.000 | 2.240 |
| 1344 | I16W_P36A_V93I_R97F | 0.020 | 4.950 | 1.000 | 2.270 | 1.000 | 2.200 |
| 1345 | I16W_P36A_V93I_R97H | 0.020 | 3.300 | 1.000 | 1.120 | 1.000 | 2.040 |
| 1346 | I16W_P36A_V93I_R97W | 1.000 | 2.340 | 0.000 | 3.690 | 0.010 | 3.290 |
| 1347 | I16W_P36A_V93K_R97H | 0.000 | 2.240 | 1.000 | 1.970 | 1.000 | −1.590 |
| 1348 | I16W_P36H | 0.000 | 6.340 | 0.000 | 5.820 | 0.000 | 5.180 |
| 1349 | I16W_P36H_R97A | 0.000 | 4.510 | 0.000 | 4.300 | 1.000 | 2.060 |
| 1350 | I16W_P36H_V93I | 0.000 | 7.130 | 0.000 | 7.200 | 0.010 | 4.240 |
| 1351 | I16W_P36H_V93I_R97A | 0.000 | 5.280 | 0.000 | 3.600 | 1.000 | 2.170 |
| 1352 | I16W_P36H_V93I_R97F | 0.000 | 3.540 | 1.000 | −0.140 | 1.000 | 0.640 |
| 1353 | I16W_P36H_V93I_R97H | 0.000 | 4.160 | 0.000 | 4.080 | 1.000 | 1.870 |
| 1354 | I16W_P36S | 0.440 | 4.710 | 0.000 | 6.500 | 0.000 | 3.170 |
| 1355 | I16W_P36S_P90W_V93I_R97A | 0.000 | 3.150 | 0.060 | 2.420 | 0.000 | 2.120 |
| 1356 | I16W_P36S_R97A | 0.000 | 4.550 | 1.000 | 3.350 | 1.000 | 1.570 |
| 1357 | I16W_P36S_V93I | 0.000 | 7.980 | 0.000 | 6.880 | 0.000 | 7.570 |
| 1358 | I16W_P36S_V93I_R97A | 0.260 | 4.800 | 0.000 | 4.700 | 1.000 | 2.420 |
| 1359 | I16W_P36S_V93I_R97H | 0.000 | 3.590 | 0.000 | 2.890 | 1.000 | 0.120 |
| 1360 | I16W_P36S_V93R_R97A | 0.000 | 2.160 | 0.000 | 2.240 | 1.000 | 0.840 |
| 1361 | I16W_P90F | 0.000 | 7.180 | 0.000 | 6.530 | 0.000 | 6.430 |
| 1362 | I16W_P90F_R97A | 0.000 | 4.440 | 0.060 | 2.640 | 0.140 | 3.340 |
| 1363 | I16W_P90F_V93I | 0.000 | 5.890 | 0.000 | 4.720 | 0.000 | 5.310 |
| 1364 | I16W_P90F_V93I_R97A | 0.000 | 5.090 | 0.000 | 4.570 | 0.000 | 3.670 |
| 1365 | I16W_P90F_V93I_R97F | 0.000 | 1.960 | 0.010 | 2.370 | 1.000 | 1.240 |
| 1366 | I16W_P90F_V93I_R97H | 0.020 | 3.100 | 0.260 | 2.750 | 0.120 | 1.190 |
| 1367 | I16W_P90F_V93I_R97W | 0.000 | 2.680 | 1.000 | 1.240 | 1.000 | 0.860 |
| 1368 | I16W_P90F_V93R_R97F | 0.010 | 3.490 | 1.000 | 0.340 | 0.160 | 1.080 |
| 1369 | I16W_P90I | 0.000 | 5.520 | 0.000 | 5.090 | 0.050 | 3.470 |
| 1370 | I16W_P90I_R97A | 0.000 | 3.960 | 0.000 | 4.070 | 0.000 | 3.500 |
| 1371 | I16W_P90I_V93I | 0.000 | 5.560 | 0.000 | 6.570 | 0.000 | 6.880 |
| 1372 | I16W_P90I_V93I_R97A | 0.000 | 2.890 | 0.720 | 3.000 | 1.000 | 0.150 |
| 1373 | I16W_P90I_V93I_R97F | 0.000 | 3.870 | 0.020 | 2.410 | 1.000 | 1.620 |
| 1374 | I16W_P90I_V93I_R97H | 0.000 | 5.560 | 1.000 | 1.990 | 1.000 | 1.400 |
| 1375 | I16W_P90W | 0.000 | 3.980 | 0.000 | 3.480 | 0.000 | 3.410 |
| 1376 | I16W_P90W_R97F | 0.020 | 2.190 | 1.000 | 0.840 | 1.000 | 0.680 |
| 1377 | I16W_P90W_V93I | 0.000 | 3.160 | 0.000 | 2.620 | 0.000 | 2.300 |
| 1378 | I16W_P90Y | 0.000 | 2.960 | 0.050 | 1.830 | 0.010 | 1.590 |
| 1379 | I16W_P90Y_V93I | 0.000 | 5.910 | 0.000 | 3.860 | 0.000 | 4.400 |
| 1380 | I16W_P90Y_V93I_R97A | 0.000 | 5.060 | 0.000 | 4.940 | 0.000 | 4.220 |
| 1381 | I16W_P90Y_V93I_R97H | 0.000 | 2.410 | 0.000 | 2.610 | 0.000 | 1.840 |
| 1382 | I16W_P90Y_V93K_R97A | 0.000 | 3.800 | 0.000 | 3.520 | 0.000 | 3.070 |
| 1383 | I16W_P90Y_V93Q_R97H | 0.000 | 2.320 | 0.500 | 1.800 | 0.000 | 3.190 |
| 1384 | I16W_R97A | 0.000 | 5.840 | 0.000 | 7.130 | 0.000 | 4.870 |
| 1385 | I16W_R97F | 0.000 | 6.660 | 0.000 | 6.590 | 0.000 | 4.790 |
| 1386 | I16W_R97H | 0.000 | 9.570 | 0.000 | 9.810 | 0.000 | 7.910 |
| 1387 | I16W_R97W | 0.000 | 7.820 | 0.000 | 7.190 | 0.000 | 6.160 |
| 1388 | I16W_V93I | 0.000 | 10.890 | 0.000 | 8.000 | 0.000 | 7.920 |
| 1389 | I16W_V93I_R97A | 0.000 | 7.240 | 0.000 | 7.750 | 0.000 | 5.530 |
| 1390 | I16W_V93I_R97F | 0.000 | 6.930 | 0.000 | 7.590 | 0.000 | 5.010 |
| 1391 | I16W_V93I_R97H | 0.000 | 7.370 | 0.000 | 7.600 | 0.000 | 6.540 |
| 1392 | I16W_V93I_R97W | 0.000 | 7.470 | 0.000 | 7.740 | 0.000 | 6.480 |
| 1393 | I16W_V93Q | 0.000 | 6.450 | 0.000 | 7.130 | 0.000 | 6.140 |
| 1394 | I16W_V93Q_R97A | 0.000 | 5.300 | 0.000 | 5.560 | 0.000 | 5.180 |
| 1395 | I16W_V93Q_R97W | 1.000 | 1.790 | 0.000 | 3.370 | 1.000 | 1.030 |
| 1396 | I16W_V93R | 0.000 | 8.220 | 0.000 | 7.760 | 0.000 | 6.240 |
| 1397 | I16W_V93R_R97A | 0.000 | 5.140 | 0.000 | 4.390 | 0.020 | 4.150 |
| 1398 | I16W_V93R_R97H | 1.000 | 1.050 | 0.000 | 4.480 | 1.000 | 1.050 |
| 1399 | I16W_V93R_R97W | 1.000 | 0.910 | 0.000 | 2.560 | 1.000 | −0.560 |
| 1400 | P36A | 0.000 | 3.524 | 0.000 | 2.480 | 0.000 | 2.036 |
| 1401 | P36A_P90F | 0.000 | 8.630 | 0.000 | 8.370 | 0.000 | 7.600 |
| 1402 | P36A_P90F_R97A | 0.000 | 3.590 | 0.000 | 3.950 | 0.000 | 3.170 |
| 1403 | P36A_P90F_R97F | 0.000 | 3.550 | 0.000 | 4.250 | 1.000 | 1.700 |
| 1404 | P36A_P90F_V93I | 0.000 | 10.000 | 0.000 | 10.370 | 0.000 | 7.910 |
| 1405 | P36A_P90F_V93I_R97A | 0.000 | 5.530 | 0.000 | 5.620 | 0.000 | 3.560 |
| 1406 | P36A_P90F_V93I_R97F | 0.000 | 5.190 | 0.000 | 4.770 | 1.000 | 1.880 |
| 1407 | P36A_P90F_V93I_R97H | 0.000 | 5.530 | 0.000 | 4.750 | 0.000 | 3.340 |
| 1408 | P36A_P90F_V93I_R97W | 0.010 | 2.820 | 0.000 | 1.840 | 1.000 | 0.390 |
| 1409 | P36A_P90F_V93K | 0.000 | 4.400 | 0.010 | 2.310 | 1.000 | 1.360 |
| 1410 | P36A_P90F_V93Q | 0.000 | 4.490 | 0.000 | 4.410 | 0.000 | 3.160 |
| 1411 | P36A_P90F_V93R | 0.010 | 3.750 | 0.420 | 2.910 | 1.000 | 2.330 |
| 1412 | P36A_P90F_V93R_R97A | 0.000 | 3.220 | 1.000 | −0.620 | 1.000 | 1.310 |
| 1413 | P36A_P90F_V93R_R97F | 0.000 | 3.080 | 0.000 | 2.780 | 1.000 | −0.680 |
| 1414 | P36A_P90I | 0.000 | 5.660 | 0.000 | 3.910 | 1.000 | 1.960 |
| 1415 | P36A_P90I_R97A | 0.000 | 6.400 | 0.040 | 2.730 | 1.000 | 0.580 |
| 1416 | P36A_P90I_R97F | 0.000 | 4.610 | 0.020 | 3.100 | 0.790 | 2.850 |
| 1417 | P36A_P90I_V93I | 0.000 | 4.990 | 0.330 | 2.250 | 0.010 | 1.300 |
| 1418 | P36A_P90I_V93I_R97A | 0.000 | 4.510 | 0.000 | 4.560 | 1.000 | 1.450 |
| 1419 | P36A_P90I_V93I_R97F | 0.000 | 3.860 | 0.000 | 4.310 | 1.000 | 1.300 |
| 1420 | P36A_P90I_V93I_R97H | 0.000 | 2.700 | 1.000 | 3.060 | 0.040 | 2.780 |
| 1421 | P36A_P90I_V93I_R97W | 0.000 | 3.960 | 0.000 | 3.270 | 1.000 | 1.130 |
| 1422 | P36A_P90I_V93K | 0.030 | 3.290 | 1.000 | 2.710 | 1.000 | −0.180 |
| 1423 | P36A_P90I_V93K_R97F | 1.000 | 0.930 | 0.000 | 2.430 | 1.000 | −1.170 |
| 1424 | P36A_P90I_V93Q | 0.120 | 3.220 | 0.000 | 3.780 | 1.000 | 0.660 |
| 1425 | P36A_P90I_V93R | 1.000 | 1.790 | 0.000 | 4.050 | 1.000 | 0.890 |
| 1426 | P36A_P90I_V93R_R97A | 1.000 | 1.490 | 0.020 | 2.590 | 1.000 | −0.690 |
| 1427 | P36A_P90I_V93R_R97H | 0.270 | 2.010 | 0.000 | 2.080 | 1.000 | 0.950 |
| 1428 | P36A_P90S_R97A | 0.000 | 3.350 | 1.000 | −2.840 | 1.000 | −0.680 |
| 1429 | P36A_P90S_R97F | 0.000 | 2.490 | 1.000 | 2.020 | 0.090 | 2.250 |
| 1430 | P36A_P90S_V93I_R97A | 0.000 | 3.910 | 1.000 | 0.060 | 1.000 | −2.250 |
| 1431 | P36A_P90S_V93I_R97F | 0.000 | 2.890 | 1.000 | 1.130 | 1.000 | −0.940 |
| 1432 | P36A_P90S_V93I_R97H | 0.000 | 3.090 | 1.000 | −1.410 | 1.000 | −1.470 |
| 1433 | P36A_P90S_V93R_R97F | 0.020 | 2.110 | 1.000 | −0.350 | 1.000 | −1.670 |
| 1434 | P36A_P90W | 0.000 | 7.660 | 0.000 | 7.890 | 0.000 | 6.500 |
| 1435 | P36A_P90W_R97F | 0.000 | 4.060 | 0.000 | 2.780 | 1.000 | 1.830 |
| 1436 | P36A_P90W_V93I | 0.000 | 8.510 | 0.000 | 7.240 | 0.000 | 6.160 |
| 1437 | P36A_P90W_V93I_R97A | 0.160 | 3.010 | 0.000 | 5.490 | 1.000 | 2.320 |
| 1438 | P36A_P90W_V93I_R97F | 0.000 | 4.840 | 0.000 | 5.590 | 0.000 | 3.360 |
| 1439 | P36A_P90W_V93I_R97H | 0.000 | 4.600 | 0.000 | 4.320 | 0.020 | 3.150 |
| 1440 | P36A_P90W_V93K_R97F | 1.000 | 0.120 | 0.000 | 2.670 | 1.000 | −2.780 |
| 1441 | P36A_P90W_V93K_R97W | 0.000 | 2.260 | 0.040 | 1.930 | 1.000 | −1.860 |
| 1442 | P36A_P90W_V93Q | 0.000 | 3.960 | 0.000 | 4.150 | 1.000 | 0.250 |
| 1443 | P36A_P90W_V93Q_R97W | 0.010 | 2.640 | 1.000 | 0.360 | 1.000 | −1.190 |
| 1444 | P36A_P90W_V93R_R97A | 0.000 | 3.290 | 0.000 | 4.430 | 1.000 | 1.780 |
| 1445 | P36A_P90W_V93R_R97W | 0.000 | 3.250 | 1.000 | 0.570 | 1.000 | −1.620 |
| 1446 | P36A_P90Y | 0.020 | 5.070 | 0.000 | 7.510 | 0.000 | 4.280 |
| 1447 | P36A_P90Y_R97F | 1.000 | 2.960 | 0.000 | 3.370 | 1.000 | 2.100 |
| 1448 | P36A_P90Y_R97H | 0.000 | 3.800 | 1.000 | 2.100 | 1.000 | 1.620 |
| 1449 | P36A_P90Y_V93I | 0.000 | 7.510 | 0.000 | 7.020 | 0.000 | 6.130 |
| 1450 | P36A_P90Y_V93I_R97A | 0.110 | 3.060 | 0.000 | 4.530 | 1.000 | 2.720 |
| 1451 | P36A_P90Y_V93I_R97F | 0.000 | 4.350 | 1.000 | 1.080 | 1.000 | 2.110 |
| 1452 | P36A_P90Y_V93I_R97H | 0.000 | 3.960 | 0.000 | 3.090 | 1.000 | 0.250 |
| 1453 | P36A_P90Y_V93R | 0.000 | 3.250 | 0.440 | 2.500 | 1.000 | −0.340 |
| 1454 | P36A_R97A | 0.000 | 3.970 | 0.100 | 2.050 | 1.000 | 0.960 |
| 1455 | P36A_R97F | 0.000 | 4.030 | 1.000 | 0.960 | 1.000 | −0.230 |
| 1456 | P36A_V93E | 0.000 | 3.930 | 0.900 | 2.570 | 1.000 | −3.020 |
| 1457 | P36A_V93I | 0.000 | 4.330 | 0.000 | 2.710 | 0.000 | 2.610 |
| 1458 | P36A_V93I_R97A | 0.000 | 3.290 | 0.000 | 1.820 | 0.000 | 1.700 |
| 1459 | P36A_V93I_R97F | 0.010 | 3.660 | 1.000 | −0.190 | 1.000 | −0.970 |
| 1460 | P36A_V93I_R97W | 0.000 | 3.260 | 0.000 | 1.980 | 0.020 | 1.310 |
| 1461 | P36A_V93I_R119Q | 1.000 | −0.230 | 0.000 | 3.150 | 1.000 | −0.340 |
| 1462 | P36A_V93K | 0.000 | 5.400 | 0.000 | 6.380 | 0.000 | 4.120 |
| 1463 | P36A_V93K_R97A | 0.000 | 3.490 | 0.000 | 4.080 | 1.000 | −0.850 |
| 1464 | P36A_V93K_R97A_R119Q | 0.000 | 2.220 | 1.000 | 1.420 | 1.000 | −1.640 |
| 1465 | P36A_V93K_R97H | 0.000 | 2.720 | 0.000 | 2.450 | 1.000 | −2.520 |
| 1466 | P36A_V93K_R119Q | 0.000 | 3.430 | 1.000 | 1.990 | 1.000 | −3.690 |
| 1467 | P36A_V93Q | 0.000 | 5.290 | 0.000 | 3.870 | 0.000 | 1.960 |
| 1468 | P36A_V93Q_R97W | 0.000 | 3.730 | 0.000 | 4.280 | 1.000 | 0.490 |
| 1469 | P36A_V93R | 0.000 | 5.250 | 0.000 | 5.530 | 1.000 | 1.440 |
| 1470 | P36A_V93R_R97A | 0.000 | 4.250 | 0.000 | 5.370 | 1.000 | 2.440 |
| 1471 | P36A_V93R_R97A_R119M | 1.000 | 1.190 | 0.020 | 2.700 | 1.000 | −1.360 |
| 1472 | P36A_V93R_R97F | 0.000 | 3.550 | 0.000 | 3.800 | 1.000 | −0.830 |
| 1473 | P36A_V93R_R97H | 0.090 | 1.540 | 0.000 | 4.910 | 1.000 | −0.330 |
| 1474 | P36A_V93R_R97W | 0.000 | 4.810 | 0.000 | 4.610 | 1.000 | 0.590 |
| 1475 | P36A_V93R_R119Q | 0.000 | 3.640 | 0.000 | 3.800 | 1.000 | 2.410 |
| 107 | P36H | 0.000 | 8.999 | 0.000 | 8.238 | 0.000 | 6.392 |
| 1476 | P36H_P90F | 0.000 | 5.880 | 0.010 | 5.360 | 0.000 | 4.110 |
| 1477 | P36H_P90F_R97F | 0.060 | 2.810 | 0.010 | 4.370 | 1.000 | 0.960 |
| 1478 | P36H_P90F_V93I | 0.000 | 7.080 | 0.000 | 7.050 | 0.000 | 5.840 |
| 1479 | P36H_P90F_V93I_R97A | 0.000 | 5.560 | 0.000 | 4.980 | 1.000 | 1.770 |
| 1480 | P36H_P90F_V93I_R97W | 0.000 | 3.770 | 0.000 | 3.950 | 1.000 | 1.320 |
| 1481 | P36H_P90I | 0.000 | 6.420 | 0.000 | 5.410 | 0.050 | 3.700 |
| 1482 | P36H_P90I_R97A | 1.000 | 2.460 | 0.000 | 2.980 | 1.000 | 2.430 |
| 1483 | P36H_P90I_V93I | 0.000 | 5.760 | 0.000 | 5.590 | 0.000 | 4.490 |
| 1484 | P36H_P90I_V93I_R97H | 0.000 | 2.940 | 0.000 | 2.590 | 1.000 | 1.200 |
| 1485 | P36H_P90I_V93I_R97W | 0.000 | 2.970 | 1.000 | 1.090 | 1.000 | 2.180 |
| 1486 | P36H_P90I_V93K_R97A | 1.000 | 0.730 | 0.010 | 2.700 | 1.000 | −1.140 |
| 1487 | P36H_P90I_V93R_R97F | 0.000 | 2.570 | 0.010 | 2.000 | 0.130 | 2.270 |
| 1488 | P36H_P90S_R97A | 0.000 | 3.350 | 0.000 | 2.720 | 1.000 | 0.020 |
| 1489 | P36H_P90S_V93I | 0.010 | 4.530 | 0.320 | 3.880 | 1.000 | 2.110 |
| 1490 | P36H_P90S_V93I_R97A | 0.000 | 3.060 | 1.000 | 1.190 | 1.000 | 1.320 |
| 1491 | P36H_P90S_V93I_R97W | 1.000 | 1.060 | 0.000 | 2.380 | 1.000 | −3.720 |
| 1492 | P36H_P90W | 0.000 | 7.410 | 0.000 | 6.470 | 0.010 | 3.710 |
| 1493 | P36H_P90W_R97F | 0.000 | 3.810 | 0.150 | 2.670 | 1.000 | −0.040 |
| 1494 | P36H_P90W_R97H | 0.000 | 3.530 | 0.000 | 3.410 | 1.000 | 0.170 |
| 1495 | P36H_P90W_R97W | 0.000 | 2.470 | 1.000 | 1.180 | 1.000 | 0.430 |
| 1496 | P36H_P90W_V93I | 0.000 | 6.550 | 0.000 | 6.780 | 0.000 | 3.550 |
| 1497 | P36H_P90W_V93I_R97H | 0.000 | 5.850 | 0.000 | 5.420 | 0.000 | 4.860 |
| 1498 | P36H_P90Y | 0.000 | 4.870 | 0.000 | 4.710 | 0.230 | 3.150 |
| 1499 | P36H_P90Y_V93I | 0.000 | 6.640 | 0.000 | 5.080 | 0.000 | 3.510 |
| 1500 | P36H_P90Y_V93I_R97A | 0.000 | 2.770 | 0.000 | 2.430 | 0.050 | 3.220 |
| 1501 | P36H_R97A | 0.000 | 3.980 | 0.000 | 3.590 | 0.000 | 2.900 |
| 1502 | P36H_R97F | 0.000 | 4.070 | 0.000 | 3.440 | 0.470 | 2.280 |
| 1503 | P36H_R97H | 0.290 | 3.420 | 0.000 | 5.160 | 0.010 | 3.710 |
| 1504 | P36H_R97W | 0.070 | 3.890 | 0.000 | 5.160 | 1.000 | 2.930 |
| 1505 | P36H_V93I | 0.000 | 7.280 | 0.000 | 7.100 | 0.000 | 4.830 |
| 1506 | P36H_V93I_R97A | 0.000 | 3.260 | 0.000 | 3.210 | 1.000 | 1.910 |
| 1507 | P36H_V93I_R97F | 0.000 | 5.190 | 0.140 | 4.100 | 1.000 | 2.090 |
| 1508 | P36H_V93I_R97H | 0.000 | 4.570 | 0.000 | 4.770 | 1.000 | 0.520 |
| 1509 | P36H_V93I_R97W | 0.000 | 4.440 | 0.000 | 5.280 | 1.000 | 1.940 |
| 1510 | P36H_V93K | 0.510 | 2.860 | 0.000 | 3.700 | 1.000 | 0.880 |
| 1511 | P36H_V93K_R97H | 1.000 | 0.380 | 1.000 | 1.140 | 0.000 | 3.490 |
| 1512 | P36H_V93Q | 0.000 | 6.710 | 0.000 | 6.370 | 0.000 | 4.260 |
| 1513 | P36H_V93Q_R97W | 0.000 | 2.500 | 0.010 | 2.230 | 1.000 | 0.530 |
| 1514 | P36H_V93R | 0.000 | 5.720 | 0.000 | 5.720 | 0.000 | 5.050 |
| 1515 | P36H_V93R_R97A | 0.000 | 3.060 | 0.000 | 1.700 | 1.000 | 0.930 |
| 1516 | P36H_V93R_R97F | 0.000 | 2.760 | 0.010 | 2.850 | 1.000 | 1.180 |
| 1517 | P36H_V93R_R97W | 0.000 | 4.250 | 0.000 | 3.880 | 1.000 | 1.720 |
| 114 | P36R | 0.000 | 7.043 | 0.000 | 7.090 | 0.000 | 4.869 |
| 1518 | P36R_P90F | 0.000 | 3.950 | 0.000 | 3.560 | 0.010 | 3.080 |
| 1519 | P36R_P90I | 0.000 | 4.400 | 0.000 | 5.000 | 0.030 | 3.610 |
| 1520 | P36R_P90I_V93I | 0.000 | 4.060 | 0.000 | 5.140 | 0.010 | 3.940 |
| 1521 | P36R_P90I_V93Q | 0.000 | 2.770 | 0.000 | 2.930 | 0.020 | 1.950 |
| 1522 | P36R_P90S_V93I_R97F | 0.000 | 3.150 | 1.000 | 0.690 | 1.000 | 1.220 |
| 1523 | P36R_P90Y_V93I | 0.000 | 3.780 | 0.000 | 3.200 | 0.000 | 3.280 |
| 1524 | P36R_R97A | 0.000 | 5.590 | 0.000 | 5.990 | 0.080 | 3.480 |
| 1525 | P36R_R97F | 0.000 | 4.430 | 0.000 | 3.130 | 0.000 | 3.090 |
| 1526 | P36R_R97W | 0.000 | 5.100 | 0.000 | 5.720 | 0.160 | 2.850 |
| 1527 | P36R_R119Q | 0.000 | 5.000 | 0.000 | 5.260 | 1.000 | 2.090 |
| 1528 | P36R_V93E | 0.000 | 2.970 | 0.050 | 1.630 | 1.000 | −0.110 |
| 1529 | P36R_V93I | 0.000 | 6.960 | 0.000 | 3.190 | 1.000 | 1.570 |
| 1530 | P36R_V93I_R97A | 0.000 | 5.880 | 0.000 | 5.710 | 0.000 | 3.980 |
| 1531 | P36R_V93I_R97F | 0.000 | 5.450 | 0.000 | 5.060 | 1.000 | 0.840 |
| 1532 | P36R_V93I_R97H | 0.000 | 4.620 | 0.000 | 5.380 | 1.000 | 1.500 |
| 1533 | P36R_V93I_R97W | 0.000 | 4.050 | 0.000 | 3.500 | 1.000 | 1.780 |
| 1534 | P36S | 0.000 | 5.731 | 0.000 | 5.147 | 0.000 | 4.993 |
| 1535 | P36S_P90F | 0.000 | 7.250 | 0.000 | 6.660 | 0.000 | 5.440 |
| 1536 | P36S_P90F_V93I | 0.000 | 7.760 | 0.000 | 8.200 | 0.000 | 6.320 |
| 1537 | P36S_P90F_V93I_R97A | 0.000 | 4.960 | 0.000 | 3.470 | 1.000 | 2.340 |
| 1538 | P36S_P90F_V93I_R97F | 0.000 | 3.790 | 0.000 | 3.380 | 0.000 | 3.930 |
| 1539 | P36S_P90F_V93I_R97H | 0.000 | 4.090 | 1.000 | 2.300 | 0.450 | 2.630 |
| 1540 | P36S_P90F_V93I_R97W | 0.000 | 3.070 | 0.000 | 3.410 | 1.000 | 1.770 |
| 1541 | P36S_P90I | 0.000 | 5.000 | 0.100 | 1.520 | 0.310 | 3.480 |
| 1542 | P36S_P90I_R97H | 0.000 | 2.940 | 1.000 | −0.240 | 1.000 | −0.410 |
| 1543 | P36S_P90I_V93I | 0.000 | 5.440 | 0.910 | 1.720 | 0.280 | 1.020 |
| 1544 | P36S_P90I_V93I_R97F | 0.000 | 3.410 | 1.000 | 1.620 | 0.000 | 2.830 |
| 1545 | P36S_P90I_V93Q | 1.000 | 0.750 | 0.000 | 4.070 | 1.000 | −0.410 |
| 1546 | P36S_P90I_V93R | 0.430 | 3.120 | 0.030 | 3.240 | 1.000 | −0.070 |
| 1547 | P36S_P90S_V93R_R97F | 1.000 | −1.490 | 0.000 | 2.110 | 1.000 | −5.310 |
| 1548 | P36S_P90W | 0.000 | 5.390 | 0.060 | 4.050 | 0.000 | 4.650 |
| 1549 | P36S_P90W_R97A | 0.000 | 3.730 | 0.000 | 3.060 | 0.000 | 3.100 |
| 1550 | P36S_P90W_V93I | 0.000 | 6.110 | 0.000 | 7.110 | 0.000 | 6.590 |
| 1551 | P36S_P90W_V93I_R97A | 0.000 | 5.200 | 0.000 | 4.380 | 0.030 | 4.070 |
| 1552 | P36S_P90W_V93I_R97H | 0.000 | 3.810 | 0.030 | 3.360 | 1.000 | 2.060 |
| 1553 | P36S_P90W_V93I_R97W | 0.000 | 3.100 | 0.840 | 2.850 | 1.000 | 1.210 |
| 1554 | P36S_P90Y | 0.000 | 6.100 | 0.000 | 6.130 | 0.040 | 3.530 |
| 1555 | P36S_P90Y_V93I | 0.000 | 7.030 | 0.000 | 6.530 | 0.000 | 6.480 |
| 1556 | P36S_P90Y_V93I_R97A | 0.000 | 3.560 | 0.000 | 3.780 | 0.520 | 3.070 |
| 1557 | P36S_P90Y_V93I_R97H | 1.000 | 2.530 | 0.010 | 3.070 | 1.000 | 1.530 |
| 1558 | P36S_P90Y_V93Q | 1.000 | 2.600 | 0.000 | 2.210 | 1.000 | −0.660 |
| 1559 | P36S_P90Y_V93R_R97A | 0.000 | 2.740 | 1.000 | −1.320 | 0.110 | 1.890 |
| 1560 | P36S_R97A | 0.000 | 4.000 | 1.000 | −0.190 | 1.000 | 0.130 |
| 1561 | P36S_R97F | 0.040 | 2.710 | 1.000 | 1.220 | 1.000 | 1.000 |
| 1562 | P36S_V93I | 0.000 | 8.820 | 0.000 | 7.240 | 0.000 | 7.600 |
| 1563 | P36S_V93I_R97F | 0.000 | 3.850 | 1.000 | 0.110 | 1.000 | −0.880 |
| 1564 | P36S_V93I_R119Q | 0.000 | 3.150 | 0.130 | 1.770 | 0.000 | 3.520 |
| 1565 | P36S_V93K | 0.000 | 5.580 | 0.000 | 5.070 | 0.000 | 4.890 |
| 1566 | P36S_V93K_R97A | 1.000 | 1.130 | 0.000 | 3.700 | 1.000 | 1.820 |
| 1567 | P36S_V93Q | 0.000 | 4.170 | 1.000 | 1.240 | 1.000 | −0.980 |
| 1568 | P36S_V93Q_R97A | 0.000 | 3.920 | 1.000 | 1.870 | 1.000 | 0.200 |
| 1569 | P36S_V93Q_R97W | 1.000 | 3.070 | 0.000 | 4.840 | 1.000 | 0.780 |
| 1570 | P36S_V93R | 0.000 | 6.260 | 0.000 | 6.080 | 0.000 | 4.230 |
| 1571 | P36S_V93R_R97A | 0.010 | 3.140 | 0.680 | 3.510 | 1.000 | 1.740 |
| 1572 | P36S_V93R_R97W | 0.090 | 3.160 | 0.010 | 3.640 | 1.000 | 1.800 |
| 117 | P36W | 0.000 | 7.115 | 0.000 | 6.861 | 0.000 | 7.268 |
| 1573 | P36W_P90F | 0.000 | 7.280 | 0.000 | 7.680 | 0.000 | 6.060 |
| 1574 | P36W_P90F_R97A | 0.000 | 3.130 | 0.000 | 3.930 | 0.030 | 3.060 |
| 1575 | P36W_P90F_R97F | 1.000 | 1.560 | 0.020 | 1.950 | 0.000 | 3.270 |
| 1576 | P36W_P90F_R97H | 0.000 | 4.250 | 0.000 | 3.600 | 1.000 | 2.670 |
| 1577 | P36W_P90F_V93I | 0.000 | 8.700 | 0.000 | 8.000 | 0.000 | 7.890 |
| 1578 | P36W_P90F_V93I_R97A | 0.000 | 5.650 | 0.000 | 5.750 | 0.000 | 4.100 |
| 1579 | P36W_P90F_V93I_R97F | 0.000 | 4.900 | 0.000 | 5.330 | 0.000 | 3.990 |
| 1580 | P36W_P90F_V93I_R97W | 0.420 | 2.890 | 0.000 | 4.340 | 0.010 | 3.520 |
| 1581 | P36W_P90F_V93K | 0.000 | 3.000 | 1.000 | 1.640 | 1.000 | −0.460 |
| 1582 | P36W_P90F_V93R | 0.000 | 3.710 | 0.000 | 4.080 | 1.000 | 0.700 |
| 1583 | P36W_P90I | 0.000 | 4.750 | 0.000 | 4.280 | 0.000 | 4.340 |
| 1584 | P36W_P90I_R97F | 1.000 | 0.510 | 1.000 | 0.960 | 0.000 | 2.750 |
| 1585 | P36W_P90I_V93I | 0.000 | 6.960 | 0.000 | 5.520 | 0.000 | 6.260 |
| 1586 | P36W_P90I_V93I_R97A | 0.600 | 1.490 | 0.000 | 2.190 | 1.000 | −0.220 |
| 1587 | P36W_P90I_V93R_R97A | 1.000 | 1.410 | 0.030 | 2.540 | 1.000 | −0.970 |
| 1588 | P36W_P90I_V93R_R97W | 1.000 | 1.950 | 0.000 | 2.140 | 1.000 | −0.190 |
| 1589 | P36W_P90S | 0.000 | 2.890 | 0.090 | 2.200 | 0.000 | 4.170 |
| 1590 | P36W_P90W | 0.000 | 6.550 | 0.000 | 6.680 | 0.000 | 4.810 |
| 1591 | P36W_P90W_R97A | 0.000 | 4.790 | 0.000 | 4.880 | 0.000 | 3.640 |
| 1592 | P36W_P90W_R97H | 0.000 | 4.350 | 0.000 | 3.960 | 1.000 | 2.460 |
| 1593 | P36W_P90W_V93I | 0.000 | 9.040 | 0.000 | 8.410 | 0.000 | 7.010 |
| 1594 | P36W_P90W_V93I_R97A | 0.000 | 5.240 | 0.000 | 6.020 | 0.000 | 4.000 |
| 1595 | P36W_P90W_V93I_R97F | 0.000 | 5.150 | 0.070 | 3.240 | 0.530 | 2.890 |
| 1596 | P36W_P90W_V93I_R97H | 0.000 | 5.640 | 0.000 | 4.990 | 0.050 | 3.140 |
| 1597 | P36W_P90W_V93I_R97W | 0.000 | 4.830 | 0.540 | 3.360 | 0.520 | 2.350 |
| 1598 | P36W_P90W_V93Q_R97A | 0.000 | 2.070 | 1.000 | 1.270 | 0.000 | 1.720 |
| 1599 | P36W_P90W_V93R_R97A | 0.000 | 2.490 | 1.000 | 1.160 | 1.000 | −1.380 |
| 1600 | P36W_P90Y | 0.000 | 4.660 | 0.000 | 4.770 | 0.000 | 3.770 |
| 1601 | P36W_P90Y_V93I | 0.000 | 7.220 | 0.000 | 6.470 | 0.000 | 4.910 |
| 1602 | P36W_P90Y_V93I_R97A | 0.000 | 3.270 | 0.180 | 2.410 | 0.030 | 2.780 |
| 1603 | P36W_P90Y_V93I_R97F | 0.000 | 3.020 | 1.000 | 2.140 | 1.000 | 0.320 |
| 1604 | P36W_P90Y_V93I_R97H | 0.000 | 3.130 | 1.000 | 1.470 | 1.000 | 0.620 |
| 1605 | P36W_P90Y_V93K | 0.020 | 2.570 | 1.000 | 0.670 | 1.000 | −0.320 |
| 1606 | P36W_P90Y_V93R | 0.160 | 2.600 | 0.000 | 2.930 | 1.000 | 1.380 |
| 1607 | P36W_P90Y_V93R_R97A | 0.000 | 2.320 | 0.210 | 1.350 | 1.000 | 0.220 |
| 1608 | P36W_R97A | 0.000 | 4.760 | 0.000 | 5.130 | 0.110 | 2.550 |
| 1609 | P36W_R97F | 0.000 | 2.880 | 0.000 | 3.550 | 1.000 | 2.280 |
| 1610 | P36W_R97H | 0.000 | 3.820 | 0.490 | 2.450 | 1.000 | 2.220 |
| 1611 | P36W_R97W | 0.000 | 4.560 | 0.040 | 2.560 | 0.160 | 3.030 |
| 1612 | P36W_V93I | 0.000 | 8.970 | 0.000 | 8.620 | 0.000 | 7.950 |
| 1613 | P36W_V93I_R97A | 0.000 | 4.840 | 0.000 | 3.960 | 0.000 | 3.990 |
| 1614 | P36W_V93I_R97F | 0.000 | 4.560 | 0.000 | 4.050 | 0.000 | 3.460 |
| 1615 | P36W_V93I_R97H | 0.150 | 3.410 | 0.000 | 4.570 | 0.240 | 2.180 |
| 1616 | P36W_V93I_R97W | 0.000 | 6.000 | 0.000 | 5.340 | 0.000 | 4.680 |
| 1617 | P36W_V93Q | 0.000 | 5.040 | 0.000 | 4.590 | 0.000 | 3.230 |
| 1618 | P36W_V93R | 0.000 | 4.110 | 0.000 | 5.800 | 0.120 | 2.920 |
| 1619 | P36W_V93R_R97A | 0.000 | 4.700 | 0.000 | 4.290 | 1.000 | 1.120 |
| 1620 | P36W_V93R_R97F | 0.000 | 3.030 | 0.000 | 2.390 | 0.070 | 2.650 |
| 1621 | P36W_V93R_R97H | 0.000 | 2.680 | 0.370 | 2.810 | 1.000 | 1.680 |
| 134 | P90F | 0.000 | 8.818 | 0.000 | 7.800 | 0.000 | 7.532 |
| 1622 | P90F_R97A | 0.000 | 5.420 | 0.000 | 6.260 | 0.000 | 4.130 |
| 1623 | P90F_R97F | 0.000 | 5.330 | 0.000 | 6.500 | 0.000 | 4.390 |
| 1624 | P90F_R97H | 0.000 | 2.900 | 0.000 | 3.510 | 0.010 | 1.830 |
| 1625 | P90F_R97W | 0.000 | 4.870 | 0.000 | 4.940 | 1.000 | 1.980 |
| 1626 | P90F_V93I | 0.000 | 9.770 | 0.000 | 9.380 | 0.000 | 8.800 |
| 1627 | P90F_V93I_R97F | 0.000 | 6.510 | 0.000 | 6.250 | 0.000 | 4.480 |
| 1628 | P90F_V93I_R97H | 0.000 | 6.840 | 0.000 | 6.130 | 0.000 | 4.540 |
| 1629 | P90F_V93I_R97W | 0.000 | 6.520 | 0.000 | 6.180 | 0.000 | 3.980 |
| 1630 | P90F_V93K | 0.000 | 6.680 | 0.000 | 6.400 | 0.000 | 3.610 |
| 1631 | P90F_V93K_R97A | 0.110 | 2.800 | 0.000 | 3.980 | 1.000 | 2.030 |
| 1632 | P90F_V93K_R97F | 0.000 | 3.490 | 0.000 | 4.500 | 1.000 | 1.290 |
| 1633 | P90F_V93K_R97H | 0.000 | 2.960 | 1.000 | 0.130 | 1.000 | 0.490 |
| 1634 | P90F_V93Q | 0.000 | 6.770 | 0.000 | 6.070 | 0.000 | 4.800 |
| 1635 | P90F_V93R | 0.000 | 6.140 | 0.000 | 6.500 | 0.070 | 3.270 |
| 1636 | P90F_V93R_R97A | 0.000 | 3.920 | 0.000 | 4.510 | 0.540 | 2.970 |
| 1637 | P90F_V93R_R97A_R119Q | 1.000 | 2.460 | 0.000 | 2.860 | 1.000 | 0.410 |
| 1638 | P90F_V93R_R97F | 0.790 | 3.230 | 0.000 | 3.510 | 1.000 | −0.750 |
| 1639 | P90F_V93R_R97H | 1.000 | 2.550 | 0.000 | 3.160 | 1.000 | 0.880 |
| 137 | P90I | 0.000 | 8.341 | 0.000 | 5.020 | 0.000 | 5.274 |
| 1640 | P90I_R97A | 0.000 | 3.900 | 0.000 | 5.270 | 0.000 | 2.160 |
| 1641 | P90I_R97F | 0.000 | 5.740 | 0.000 | 4.920 | 0.060 | 3.920 |
| 1642 | P90I_R97H | 0.110 | 3.990 | 0.000 | 4.480 | 1.000 | 2.480 |
| 1643 | P90I_R97W | 0.030 | 3.470 | 0.000 | 3.920 | 0.650 | 2.740 |
| 1644 | P90I_V93I | 0.000 | 7.410 | 0.000 | 5.670 | 0.000 | 5.500 |
| 1645 | P90I_V93I_R97A | 0.000 | 5.820 | 0.000 | 5.460 | 0.000 | 4.580 |
| 1646 | P90I_V93I_R97F | 0.000 | 6.880 | 0.000 | 6.190 | 0.000 | 5.210 |
| 1647 | P90I_V93I_R97H | 0.000 | 6.120 | 0.000 | 4.820 | 0.010 | 3.850 |
| 1648 | P90I_V93I_R97W | 0.000 | 5.880 | 0.000 | 4.130 | 0.110 | 2.950 |
| 1649 | P90I_V93K | 0.000 | 7.360 | 0.000 | 7.370 | 0.180 | 3.970 |
| 1650 | P90I_V93K_R97A | 1.000 | 2.440 | 0.000 | 3.960 | 1.000 | 1.160 |
| 1651 | P90I_V93K_R97H | 1.000 | 0.140 | 0.000 | 3.430 | 1.000 | −0.440 |
| 1652 | P90I_V93K_R97P | 0.000 | 2.030 | 1.000 | 1.170 | 1.000 | 0.500 |
| 1653 | P90I_V93Q | 0.000 | 6.450 | 0.000 | 6.710 | 0.000 | 4.310 |
| 1654 | P90I_V93Q_R97A | 1.000 | 2.160 | 0.000 | 3.600 | 1.000 | −0.280 |
| 1655 | P90I_V93R | 0.000 | 7.960 | 0.000 | 8.450 | 0.000 | 5.730 |
| 1656 | P90I_V93R_R97A | 0.000 | 5.890 | 0.000 | 5.110 | 0.000 | 4.490 |
| 1657 | P90I_V93R_R97F | 1.000 | 2.130 | 0.020 | 3.820 | 1.000 | 1.420 |
| 1658 | P90I_V93R_R97H | 0.000 | 3.150 | 0.000 | 3.220 | 1.000 | −0.530 |
| 1659 | P90S_R97H | 0.020 | 3.190 | 1.000 | −1.240 | 1.000 | −1.930 |
| 1660 | P90S_R97W | 0.000 | 4.570 | 1.000 | 1.110 | 1.000 | −1.840 |
| 1661 | P90S_V93I_R97A | 0.020 | 3.580 | 1.000 | 0.080 | 1.000 | 0.620 |
| 1662 | P90S_V93I_R97F | 0.000 | 3.800 | 1.000 | 0.010 | 1.000 | −0.380 |
| 1663 | P90S_V93I_R97H | 0.000 | 4.310 | 1.000 | −1.270 | 1.000 | −1.720 |
| 1664 | P90S_V93I_R97W | 0.000 | 3.810 | 1.000 | −0.300 | 1.000 | −1.430 |
| 1665 | P90S_V93K_R97F | 0.000 | 3.670 | 0.000 | 3.280 | 1.000 | −0.280 |
| 1666 | P90S_V93K_R97W | 1.000 | 0.000 | 0.000 | 2.830 | 1.000 | −1.050 |
| 1667 | P90S_V93Q | 0.000 | 5.180 | 0.000 | 4.660 | 0.020 | 2.900 |
| 1668 | P90S_V93Q_R97A | 1.000 | 2.380 | 0.000 | 2.830 | 1.000 | −0.680 |
| 1669 | P90S_V93Q_R97F | 0.000 | 2.590 | 0.010 | 2.460 | 1.000 | 2.620 |
| 1670 | P90S_V93Q_R97W | 1.000 | −0.270 | 0.000 | 2.820 | 1.000 | −0.010 |
| 1671 | P90S_V93R | 0.000 | 5.240 | 0.000 | 5.460 | 0.000 | 3.730 |
| 1672 | P90S_V93R_R97A | 0.000 | 2.930 | 0.000 | 2.940 | 0.000 | 2.330 |
| 1673 | P90S_V93R_R97H | 0.000 | 3.180 | 0.010 | 2.330 | 1.000 | 1.860 |
| 1674 | P90S_V93R_R97W | 0.000 | 3.070 | 0.550 | 2.670 | 0.000 | 2.860 |
| 144 | P90W | 0.000 | 5.557 | 0.000 | 5.737 | 0.000 | 4.223 |
| 1675 | P90W_R97A | 0.000 | 6.370 | 0.000 | 6.510 | 0.150 | 3.020 |
| 1676 | P90W_R97F | 0.000 | 5.700 | 0.000 | 5.390 | 0.000 | 3.730 |
| 1677 | P90W_R97H | 0.000 | 6.080 | 0.000 | 5.500 | 0.000 | 4.320 |
| 1678 | P90W_R97W | 0.000 | 4.890 | 0.000 | 4.920 | 0.000 | 3.770 |
| 1679 | P90W_V93I | 0.000 | 8.770 | 0.000 | 8.170 | 0.000 | 7.990 |
| 1680 | P90W_V93I_R97A | 0.000 | 6.270 | 0.000 | 5.640 | 0.150 | 3.390 |
| 1681 | P90W_V93I_R97F | 0.000 | 6.990 | 0.000 | 5.570 | 0.000 | 3.550 |
| 1682 | P90W_V93I_R97H | 0.000 | 6.100 | 0.000 | 5.200 | 0.000 | 4.630 |
| 1683 | P90W_V93I_R97W | 0.000 | 6.240 | 0.000 | 6.550 | 0.000 | 4.600 |
| 1684 | P90W_V93K | 0.000 | 7.000 | 0.000 | 6.370 | 0.100 | 2.980 |
| 1685 | P90W_V93K_R97F | 0.000 | 2.550 | 0.000 | 3.700 | 1.000 | 2.560 |
| 1686 | P90W_V93K_R97H | 1.000 | 2.450 | 0.000 | 3.310 | 1.000 | −2.290 |
| 1687 | P90W_V93Q | 0.000 | 4.440 | 0.000 | 5.410 | 0.070 | 3.820 |
| 1688 | P90W_V93Q_R97A | 0.000 | 3.200 | 0.030 | 3.720 | 1.000 | 0.050 |
| 1689 | P90W_V93Q_R97H | 0.000 | 2.100 | 1.000 | 1.620 | 1.000 | 1.180 |
| 1690 | P90W_V93R | 0.000 | 6.780 | 0.000 | 6.640 | 0.000 | 5.030 |
| 1691 | P90W_V93R_R97A | 0.000 | 3.680 | 0.000 | 3.510 | 1.000 | 2.410 |
| 1692 | P90W_V93R_R97F | 0.100 | 3.290 | 0.020 | 3.350 | 1.000 | 2.270 |
| 1693 | P90W_V93R_R97H | 0.000 | 2.800 | 1.000 | 2.150 | 1.000 | 0.500 |
| 1694 | P90W_V93R_R97W | 0.690 | 2.400 | 0.000 | 4.110 | 0.010 | 2.920 |
| 145 | P90Y | 0.000 | 8.109 | 0.000 | 7.437 | 0.000 | 7.526 |
| 1695 | P90Y_R97A | 0.000 | 4.180 | 0.000 | 4.690 | 1.000 | 1.920 |
| 1696 | P90Y_R97F | 0.000 | 4.410 | 0.000 | 5.450 | 1.000 | 2.260 |
| 1697 | P90Y_R97H | 0.000 | 5.790 | 0.000 | 4.120 | 1.000 | 1.160 |
| 1698 | P90Y_R97W | 0.000 | 3.970 | 0.000 | 3.700 | 0.000 | 3.220 |
| 1699 | P90Y_V93I | 0.000 | 8.450 | 0.000 | 8.340 | 0.000 | 8.110 |
| 1700 | P90Y_V93I_R97A | 0.000 | 5.370 | 0.000 | 4.700 | 0.660 | 2.840 |
| 1701 | P90Y_V93I_R97F | 0.000 | 5.300 | 0.000 | 4.670 | 0.020 | 3.650 |
| 1702 | P90Y_V93I_R97H | 0.000 | 4.000 | 0.000 | 3.970 | 1.000 | 1.790 |
| 1703 | P90Y_V93I_R97W | 0.000 | 5.360 | 0.000 | 5.570 | 1.000 | 1.610 |
| 1704 | P90Y_V93K | 0.020 | 5.180 | 0.000 | 5.200 | 0.010 | 4.260 |
| 1705 | P90Y_V93Q | 0.000 | 5.830 | 0.000 | 7.520 | 0.000 | 3.740 |
| 1706 | P90Y_V93Q_R97A | 0.000 | 3.880 | 0.000 | 4.020 | 1.000 | 0.420 |
| 1707 | P90Y_V93Q_R97F | 0.000 | 2.340 | 0.070 | 3.270 | 1.000 | 0.680 |
| 1708 | P90Y_V93Q_R97W | 0.000 | 4.570 | 0.000 | 4.010 | 0.000 | 3.980 |
| 1709 | P90Y_V93R | 0.000 | 5.490 | 0.000 | 4.670 | 0.380 | 3.600 |
| 1710 | P90Y_V93R_R97F | 0.000 | 3.230 | 1.000 | 1.010 | 1.000 | 1.940 |
| 182 | R97A | 0.000 | 3.210 | 0.092 | 1.551 | 1.000 | 0.827 |
| 1711 | R97A_R119M | 0.090 | 1.900 | 0.030 | 2.740 | 1.000 | −1.090 |
| 186 | R97F | 0.000 | 4.672 | 1.000 | 0.858 | 1.000 | 0.326 |
| 199 | R97W | 0.000 | 4.414 | 0.251 | 1.312 | 1.000 | 1.233 |
| 257 | R119Q | 0.003 | 3.224 | 0.002 | 3.792 | 1.000 | 2.172 |
| 1712 | V93I | 0.000 | 3.625 | 0.000 | 2.086 | 0.003 | 1.787 |
| 1713 | V93I_R97A | 0.000 | 2.230 | 1.000 | 0.680 | 1.000 | 0.400 |
| 1714 | V93I_R97A_R119M | 0.000 | 2.680 | 0.030 | 2.050 | 1.000 | 1.410 |
| 1715 | V93I_R97A_R119Q | 0.000 | 4.190 | 1.000 | 1.560 | 1.000 | −2.130 |
| 1716 | V93I_R97F | 0.000 | 3.260 | 0.000 | 2.170 | 0.060 | 1.350 |
| 1717 | V93I_R97F_R119Q | 0.000 | 2.960 | 0.000 | 2.600 | 1.000 | −0.130 |
| 1718 | V93I_R97H | 0.000 | 4.130 | 0.090 | 1.400 | 1.000 | 0.850 |
| 1719 | V93I_R97W | 0.000 | 4.960 | 0.000 | 2.350 | 0.000 | 1.700 |
| 1720 | V93I_R119Q | 0.540 | 3.460 | 0.010 | 3.980 | 1.000 | 0.400 |
| 1721 | V93I_R119Y | 1.000 | −4.120 | 0.000 | 2.430 | 1.000 | −0.500 |
| 158 | V93K | 0.000 | 5.716 | 0.000 | 6.820 | 0.000 | 3.864 |
| 1722 | V93K_R97A | 0.000 | 3.910 | 0.000 | 4.060 | 0.010 | 2.940 |
| 1723 | V93K_R97A_R119M | 0.000 | 3.020 | 1.000 | 1.090 | 1.000 | −4.570 |
| 1724 | V93K_R97F | 0.010 | 3.320 | 0.000 | 4.320 | 0.330 | 2.720 |
| 1725 | V93K_R97F_R119Q | 1.000 | −0.040 | 0.000 | 2.520 | 1.000 | −0.440 |
| 1726 | V93K_R97H | 0.000 | 3.940 | 0.000 | 3.660 | 1.000 | 1.080 |
| 1727 | V93K_R97W | 0.000 | 4.530 | 0.500 | 2.720 | 1.000 | 0.310 |
| 1728 | V93K_R119M | 0.000 | 3.020 | 1.000 | 2.030 | 1.000 | −2.250 |
| 1729 | V93K_R119Q | 0.000 | 2.590 | 1.000 | 1.730 | 0.660 | 2.780 |
| 162 | V93Q | 0.000 | 2.985 | 0.000 | 1.845 | 0.399 | 1.529 |
| 1730 | V93Q_R97A | 0.000 | 3.450 | 0.000 | 4.270 | 0.080 | 2.810 |
| 1731 | V93Q_R97H | 0.190 | 2.500 | 0.010 | 4.250 | 1.000 | 1.880 |
| 1732 | V93Q_R97P | 1.000 | 1.580 | 0.000 | 2.510 | 1.000 | 0.080 |
| 1733 | V93Q_R97W | 0.000 | 3.980 | 0.000 | 3.400 | 1.000 | 1.340 |
| 163 | V93R | 0.000 | 9.695 | 0.000 | 9.313 | 0.000 | 8.157 |
| 1734 | V93R_R97A | 0.000 | 5.770 | 0.000 | 5.630 | 0.450 | 2.700 |
| 1735 | V93R_R97A_R119E | 0.000 | 2.150 | 1.000 | −2.970 | 1.000 | −4.820 |
| 1736 | V93R_R97A_R119Y | 0.000 | 3.090 | 0.000 | 3.590 | 1.000 | 0.550 |
| 1737 | V93R_R97F | 0.000 | 4.380 | 0.000 | 4.200 | 0.230 | 1.130 |
| 1738 | V93R_R97H | 0.000 | 4.980 | 0.000 | 5.390 | 0.000 | 4.020 |
| 1739 | V93R_R97H_R119M | 0.000 | 3.360 | 0.000 | 2.750 | 1.000 | −2.650 |
| 1740 | V93R_R97W | 0.000 | 4.590 | 0.000 | 4.210 | 1.000 | 1.650 |
| 1741 | V93R_R119Q | 0.000 | 4.260 | 0.000 | 4.330 | 1.000 | 2.370 |
| 1742 | Y7C_I16F | 0.000 | 3.300 | 0.700 | 1.090 | 0.020 | 2.520 |
| 1743 | Y7C_I16F_P36A_P90W_V93I | 0.010 | 3.110 | 0.000 | 4.400 | 0.200 | 2.830 |
| 1744 | Y7C_I16F_P36A_P90Y_V93I | 0.040 | 3.430 | 0.210 | 2.620 | 0.000 | 3.520 |
| 1745 | Y7C_I16F_P36A_P90Y_V93I_R97A | 1.000 | 0.830 | 1.000 | 1.910 | 0.000 | 2.670 |
| 1746 | Y7C_I16F_P36A_V93I | 0.000 | 2.020 | 1.000 | −0.150 | 0.190 | 1.110 |
| 1747 | Y7C_I16F_P36A_V93I_R97F | 0.000 | 3.760 | 1.000 | 0.210 | 1.000 | 0.330 |
| 1748 | Y7C_I16F_P36H_P90Y_V93I | 0.000 | 4.110 | 0.010 | 2.920 | 0.000 | 3.090 |
| 1749 | Y7C_I16F_P36S_P90F_V93I | 0.020 | 2.510 | 1.000 | −0.480 | 1.000 | −1.520 |
| 1750 | Y7C_I16F_P36S_P90F_V93I_R97A | 0.000 | 2.890 | 1.000 | −3.980 | 1.000 | −1.730 |
| 1751 | Y7C_I16F_P36W_P90F | 0.000 | 2.240 | 1.000 | 1.600 | 1.000 | 1.130 |
| 1752 | Y7C_I16F_P36W_P90F_V93I | 0.000 | 2.690 | 1.000 | 1.190 | 1.000 | −0.100 |
| 1753 | Y7C_I16F_P36W_P90F_V93I_R97A | 0.000 | 3.020 | 1.000 | 0.360 | 1.000 | −0.190 |
| 1754 | Y7C_I16F_P90F | 0.000 | 6.860 | 0.000 | 4.850 | 0.000 | 5.360 |
| 1755 | Y7C_I16F_P90F_R97A | 0.160 | 2.890 | 0.000 | 2.890 | 1.000 | 2.130 |
| 1756 | Y7C_I16F_P90F_R97F | 0.020 | 3.320 | 0.450 | 1.840 | 1.000 | 2.600 |
| 1757 | Y7C_I16F_P90F_V93I | 0.000 | 7.480 | 0.000 | 6.680 | 0.000 | 7.480 |
| 1758 | Y7C_I16F_P90F_V93I_R97A | 0.000 | 4.180 | 0.080 | 3.410 | 1.000 | 1.890 |
| 1759 | Y7C_I16F_P90F_V93I_R97F | 0.000 | 4.140 | 0.000 | 3.880 | 0.150 | 3.170 |
| 1760 | Y7C_I16F_P90F_V93I_R97H | 0.000 | 4.980 | 0.000 | 3.510 | 1.000 | 1.320 |
| 1761 | Y7C_I16F_P90F_V93I_R97W | 0.000 | 5.780 | 0.000 | 5.100 | 0.000 | 4.940 |
| 1762 | Y7C_I16F_P90I | 0.000 | 7.060 | 0.000 | 5.250 | 0.000 | 5.910 |
| 1763 | Y7C_I16F_P90I_V93I | 0.000 | 6.020 | 0.000 | 5.950 | 0.000 | 5.670 |
| 1764 | Y7C_I16F_P90I_V93I_R97A | 1.000 | 2.960 | 0.000 | 3.130 | 1.000 | 0.520 |
| 1765 | Y7C_I16F_P90I_V93I_R97F | 0.000 | 3.310 | 0.000 | 2.760 | 0.070 | 2.480 |
| 1766 | Y7C_I16F_P90I_V93I_R97H | 1.000 | 1.290 | 0.040 | 3.180 | 1.000 | 1.260 |
| 1767 | Y7C_I16F_P90I_V93R | 0.000 | 5.170 | 0.000 | 3.650 | 1.000 | 2.510 |
| 1768 | Y7C_I16F_P90S_R97A | 0.000 | 3.830 | 0.040 | 3.020 | 0.000 | 3.890 |
| 1769 | Y7C_I16F_P90W | 0.000 | 7.340 | 0.000 | 5.870 | 0.000 | 5.240 |
| 1770 | Y7C_I16F_P90W_R97A | 0.000 | 5.470 | 0.000 | 4.790 | 1.000 | 2.000 |
| 1771 | Y7C_I16F_P90W_R97F | 1.000 | 1.680 | 0.000 | 3.360 | 1.000 | 1.680 |
| 1772 | Y7C_I16F_P90W_R97H | 0.370 | 2.750 | 0.000 | 3.600 | 1.000 | 0.870 |
| 1773 | Y7C_I16F_P90W_R97W | 0.000 | 4.630 | 0.080 | 3.150 | 1.000 | 1.700 |
| 1774 | Y7C_I16F_P90W_V93I | 0.000 | 7.030 | 0.000 | 6.310 | 0.000 | 7.030 |
| 1775 | Y7C_I16F_P90W_V93I_R97A | 0.000 | 5.250 | 0.000 | 4.020 | 0.290 | 2.070 |
| 1776 | Y7C_I16F_P90W_V93I_R97F | 0.000 | 4.500 | 0.000 | 5.100 | 1.000 | 2.450 |
| 1777 | Y7C_I16F_P90W_V93I_R97H | 0.000 | 3.860 | 0.000 | 4.320 | 0.580 | 2.030 |
| 1778 | Y7C_I16F_P90W_V93I_R97W | 0.540 | 2.220 | 0.000 | 3.070 | 1.000 | 0.770 |
| 1779 | Y7C_I16F_P90Y | 0.000 | 5.550 | 0.000 | 4.420 | 0.000 | 5.810 |
| 1780 | Y7C_I16F_P90Y_R97A | 0.000 | 4.440 | 0.000 | 3.260 | 0.030 | 3.140 |
| 1781 | Y7C_I16F_P90Y_V93I | 0.000 | 7.370 | 0.000 | 5.080 | 0.000 | 6.850 |
| 1782 | Y7C_I16F_P90Y_V93I_R97A | 0.000 | 4.500 | 1.000 | 2.700 | 0.410 | 3.190 |
| 1783 | Y7C_I16F_P90Y_V93I_R97F | 0.000 | 3.160 | 0.000 | 4.050 | 1.000 | 2.040 |
| 1784 | Y7C_I16F_P90Y_V93I_R97H | 0.020 | 2.740 | 0.000 | 3.450 | 1.000 | 1.960 |
| 1785 | Y7C_I16F_P90Y_V93I_R97W | 1.000 | 2.390 | 0.000 | 3.170 | 1.000 | 0.630 |
| 1786 | Y7C_I16F_P90Y_V93K | 0.000 | 2.010 | 1.000 | 0.080 | 1.000 | 1.070 |
| 1787 | Y7C_I16F_P90Y_V93K_R97A | 1.000 | 0.520 | 0.830 | 1.420 | 0.000 | 2.570 |
| 1788 | Y7C_I16F_R97A | 0.000 | 2.870 | 0.000 | 2.160 | 0.000 | 2.420 |
| 1789 | Y7C_I16F_R97F | 0.000 | 4.600 | 0.000 | 2.630 | 0.000 | 3.610 |
| 1790 | Y7C_I16F_R97W | 0.000 | 3.070 | 1.000 | 0.830 | 1.000 | 0.060 |
| 1791 | Y7C_I16F_V93I | 0.000 | 4.030 | 0.180 | 1.610 | 0.000 | 1.800 |
| 1792 | Y7C_I16F_V93I_R97A | 1.000 | 2.090 | 0.090 | 3.270 | 0.000 | 4.750 |
| 1793 | Y7C_I16F_V93I_R97F | 0.000 | 4.330 | 0.070 | 3.630 | 0.530 | 2.800 |
| 1794 | Y7C_I16F_V93I_R97H | 0.000 | 3.530 | 1.000 | 2.930 | 0.000 | 4.280 |
| 1795 | Y7C_I16F_V93I_R97W | 0.000 | 4.170 | 0.000 | 3.990 | 0.020 | 3.800 |
| 1796 | Y7C_I16F_V93Q | 0.000 | 3.540 | 0.100 | 2.370 | 0.090 | 2.900 |
| 1797 | Y7C_I16F_V93Q_R97A | 0.000 | 3.220 | 0.070 | 2.380 | 1.000 | 0.610 |
| 1798 | Y7C_I16F_V93R | 0.000 | 4.540 | 0.010 | 3.220 | 0.000 | 3.000 |
| 1799 | Y7C_I16F_V93R_R97A | 0.000 | 4.970 | 0.000 | 4.590 | 0.000 | 4.420 |
| 1800 | Y7C_I16F_V93R_R97A_R119Q | 0.010 | 2.180 | 1.000 | 0.270 | 1.000 | 0.680 |
| 1801 | Y7C_I16F_V93R_R97F | 0.000 | 3.410 | 0.150 | 2.570 | 0.070 | 2.750 |
| 1802 | Y7C_I16F_V93R_R97W | 0.940 | 2.250 | 0.000 | 2.360 | 1.000 | −1.300 |
| 1803 | Y7C_I16L_P36A_P90F_V93I | 0.000 | 2.710 | 1.000 | 2.220 | 0.180 | 2.590 |
| 1804 | Y7C_I16L_P36A_P90W_V93I | 0.370 | 2.980 | 0.040 | 2.730 | 1.000 | 0.270 |
| 1805 | Y7C_I16L_P36A_P90W_V93I_R97A | 0.000 | 2.170 | 1.000 | 0.770 | 1.000 | 0.470 |
| 1806 | Y7C_I16L_P36A_P90Y_R97H | 0.000 | 2.470 | 0.250 | 1.770 | 1.000 | −1.640 |
| 1807 | Y7C_I16L_P36A_P90Y_V93I | 0.010 | 2.950 | 1.000 | 2.710 | 0.010 | 4.100 |
| 1808 | Y7C_I16L_P36A_P90Y_V93Q | 0.000 | 2.420 | 1.000 | −4.540 | 1.000 | −3.530 |
| 1809 | Y7C_I16L_P36H_P90W_V93I | 0.010 | 5.200 | 1.000 | 0.230 | 1.000 | 2.020 |
| 1810 | Y7C_I16L_P36H_V93I | 0.000 | 3.100 | 1.000 | 0.110 | 1.000 | −1.270 |
| 1811 | Y7C_I16L_P36S_P90F | 0.010 | 2.740 | 1.000 | −0.410 | 1.000 | 0.760 |
| 1812 | Y7C_I16L_P36S_P90W | 1.000 | 0.130 | 0.000 | 2.100 | 1.000 | 1.420 |
| 1813 | Y7C_I16L_P36W_P90F | 0.000 | 1.740 | 1.000 | 0.770 | 0.000 | 3.320 |
| 1814 | Y7C_I16L_P36W_P90F_R97A | 0.000 | 2.070 | 1.000 | 1.070 | 1.000 | 0.230 |
| 1815 | Y7C_I16L_P36W_P90F_V93I | 0.080 | 3.180 | 0.500 | 2.140 | 0.030 | 3.260 |
| 1816 | Y7C_I16L_P36W_P90F_V93I_R97F | 0.000 | 2.330 | 0.260 | 1.400 | 1.000 | −1.470 |
| 1817 | Y7C_I16L_P36W_P90W_V93I | 0.000 | 5.130 | 0.000 | 4.200 | 0.000 | 4.550 |
| 1818 | Y7C_I16L_P36W_P90W_V93I_R97A | 1.000 | 1.390 | 0.010 | 2.110 | 1.000 | 0.130 |
| 1819 | Y7C_I16L_P36W_P90Y | 0.000 | 2.390 | 1.000 | 0.710 | 1.000 | 2.040 |
| 1820 | Y7C_I16L_P36W_P90Y_V93I | 0.000 | 4.510 | 0.000 | 3.300 | 0.200 | 2.880 |
| 1821 | Y7C_I16L_P36W_V93I | 0.000 | 3.620 | 0.000 | 3.040 | 1.000 | 0.960 |
| 1822 | Y7C_I16L_P90F | 0.000 | 4.000 | 1.000 | 1.770 | 0.000 | 3.050 |
| 1823 | Y7C_I16L_P90F_V93I | 0.000 | 5.270 | 0.000 | 4.160 | 0.000 | 3.820 |
| 1824 | Y7C_I16L_P90F_V93I_R97A | 0.000 | 3.870 | 1.000 | 1.230 | 1.000 | 2.120 |
| 1825 | Y7C_I16L_P90F_V93I_R97W | 1.000 | 2.620 | 0.000 | 3.960 | 1.000 | 0.850 |
| 1826 | Y7C_I16L_P90F_V93Q | 0.000 | 3.580 | 0.000 | 3.900 | 1.000 | 0.810 |
| 1827 | Y7C_I16L_P90F_V93Q_R97F | 1.000 | 0.930 | 0.000 | 2.350 | 1.000 | −4.260 |
| 1828 | Y7C_I16L_P90I | 0.000 | 2.590 | 1.000 | −0.740 | 1.000 | −0.050 |
| 1829 | Y7C_I16L_P90I_V93I | 0.000 | 3.870 | 1.000 | 0.680 | 1.000 | 1.000 |
| 1830 | Y7C_I16L_P90I_V93I_R97A | 0.030 | 2.070 | 0.000 | 2.240 | 1.000 | 1.910 |
| 1831 | Y7C_I16L_P90W | 0.000 | 5.340 | 0.000 | 4.190 | 0.000 | 4.020 |
| 1832 | Y7C_I16L_P90W_R97A | 0.000 | 4.660 | 1.000 | 0.940 | 1.000 | −1.240 |
| 1833 | Y7C_I16L_P90W_R97F | 1.000 | 2.330 | 0.010 | 2.700 | 1.000 | 2.870 |
| 1834 | Y7C_I16L_P90W_R97H | 1.000 | 2.540 | 0.000 | 4.390 | 1.000 | −0.610 |
| 1835 | Y7C_I16L_P90W_V93I | 0.000 | 5.720 | 0.000 | 5.840 | 0.010 | 3.530 |
| 1836 | Y7C_I16L_P90W_V93I_R97A | 0.000 | 3.140 | 1.000 | 2.270 | 1.000 | −0.490 |
| 1837 | Y7C_I16L_P90W_V93I_R97F | 0.000 | 2.730 | 1.000 | 1.460 | 1.000 | 0.050 |
| 1838 | Y7C_I16L_P90W_V93I_R97H | 1.000 | 2.180 | 0.000 | 3.200 | 1.000 | 0.730 |
| 1839 | Y7C_I16L_P90W_V93Q | 0.040 | 3.210 | 1.000 | 2.060 | 1.000 | −1.420 |
| 1840 | Y7C_I16L_P90W_V93Q_R97A | 1.000 | 0.000 | 0.000 | 2.690 | 1.000 | −2.480 |
| 1841 | Y7C_I16L_P90W_V93R | 1.000 | 2.650 | 0.020 | 3.180 | 1.000 | −0.470 |
| 1842 | Y7C_I16L_P90W_V93R_R97A | 1.000 | 1.860 | 0.000 | 2.400 | 1.000 | −0.390 |
| 1843 | Y7C_I16L_P90Y | 0.000 | 5.520 | 0.010 | 3.150 | 0.130 | 3.440 |
| 1844 | Y7C_I16L_P90Y_V93I | 0.000 | 5.840 | 0.000 | 2.430 | 0.000 | 4.060 |
| 1845 | Y7C_I16L_P90Y_V93I_R97A | 1.000 | 0.240 | 0.000 | 3.040 | 1.000 | −0.410 |
| 1846 | Y7C_I16L_P90Y_V93I_R97F | 0.000 | 2.910 | 0.250 | 2.180 | 1.000 | −0.260 |
| 1847 | Y7C_I16L_P90Y_V93Q | 0.000 | 3.690 | 0.000 | 3.040 | 0.000 | 2.880 |
| 1848 | Y7C_I16L_V93I_R97F | 0.000 | 3.180 | 1.000 | 1.740 | 1.000 | 1.320 |
| 1849 | Y7C_I16L_V93Q | 0.000 | 3.540 | 0.000 | 3.290 | 0.000 | 3.580 |
| 1850 | Y7C_I16L_V93R | 0.000 | 5.160 | 0.000 | 3.780 | 0.000 | 3.320 |
| 1851 | Y7C_I16L_V93R_R97W | 0.000 | 2.760 | 0.000 | 2.470 | 1.000 | −0.190 |
| 1852 | Y7C_I16L_V93R_R119Q | 0.300 | 2.360 | 0.010 | 2.530 | 0.000 | 2.060 |
| 1853 | Y7C_I16M | 0.000 | 2.340 | 1.000 | 0.870 | 0.030 | 1.480 |
| 1854 | Y7C_I16M_P36A_P90F | 0.000 | 3.210 | 1.000 | 0.560 | 1.000 | 1.990 |
| 1855 | Y7C_I16M_P36A_P90F_V93I | 0.000 | 3.100 | 0.020 | 2.220 | 0.000 | 3.440 |
| 1856 | Y7C_I16M_P36A_P90W | 0.000 | 3.290 | 0.000 | 2.440 | 0.550 | 2.250 |
| 1857 | Y7C_I16M_P36A_P90W_V93I | 0.000 | 3.690 | 1.000 | 1.940 | 1.000 | 1.220 |
| 1858 | Y7C_I16M_P36A_P90W_V93I_R97H | 1.000 | −1.520 | 0.000 | 2.730 | 1.000 | −0.130 |
| 1859 | Y7C_I16M_P36A_V93R_R97A | 0.000 | 2.160 | 1.000 | −0.860 | 1.000 | −0.590 |
| 1860 | Y7C_I16M_P36H_V93I_R97H | 0.000 | 3.330 | 0.000 | 2.790 | 0.000 | 2.510 |
| 1861 | Y7C_I16M_P36W | 0.000 | 2.920 | 0.000 | 2.830 | 1.000 | 2.060 |
| 1862 | Y7C_I16M_P36W_P90F | 0.210 | 3.210 | 0.730 | 2.440 | 0.000 | 3.910 |
| 1863 | Y7C_I16M_P36W_P90F_V93I | 0.000 | 3.210 | 0.000 | 3.050 | 0.000 | 3.010 |
| 1864 | Y7C_I16M_P36W_P90I_V93K | 0.000 | 2.640 | 1.000 | 1.130 | 1.000 | 0.870 |
| 1865 | Y7C_I16M_P36W_P90W | 0.000 | 3.710 | 0.000 | 3.840 | 0.620 | 2.530 |
| 1866 | Y7C_I16M_P36W_P90W_V93I | 0.060 | 3.860 | 0.000 | 3.270 | 0.000 | 3.600 |
| 1867 | Y7C_I16M_P36W_P90W_V93I_R97W | 0.000 | 2.700 | 1.000 | 1.070 | 1.000 | 1.160 |
| 1868 | Y7C_I16M_P36W_P90Y_V93I | 0.000 | 2.580 | 1.000 | 1.490 | 1.000 | 1.810 |
| 1869 | Y7C_I16M_P36W_V93I | 0.000 | 2.950 | 1.000 | 0.780 | 0.650 | 2.580 |
| 1870 | Y7C_I16M_P36W_V93I_R97A | 0.000 | 2.660 | 1.000 | −0.060 | 1.000 | 0.020 |
| 1871 | Y7C_I16M_P90F | 0.000 | 3.970 | 0.000 | 3.160 | 0.000 | 2.830 |
| 1872 | Y7C_I16M_P90F_V93I | 0.000 | 2.360 | 1.000 | 1.480 | 0.000 | 2.290 |
| 1873 | Y7C_I16M_P90F_V93K_R97A | 0.000 | 2.660 | 1.000 | −4.910 | 1.000 | −1.820 |
| 1874 | Y7C_I16M_P90F_V93Q | 1.000 | 2.490 | 0.000 | 3.090 | 1.000 | 0.800 |
| 1875 | Y7C_I16M_P90I_V93I | 0.000 | 3.850 | 1.000 | 0.290 | 1.000 | 1.990 |
| 1876 | Y7C_I16M_P90I_V93R | 0.000 | 4.540 | 0.000 | 4.310 | 0.000 | 3.420 |
| 1877 | Y7C_I16M_P90W | 0.000 | 3.720 | 0.030 | 2.580 | 0.020 | 2.940 |
| 1878 | Y7C_I16M_P90W_R97H | 1.000 | 0.220 | 0.020 | 2.090 | 1.000 | 1.490 |
| 1879 | Y7C_I16M_P90W_V93I | 0.570 | 3.090 | 0.340 | 2.770 | 0.010 | 2.580 |
| 1880 | Y7C_I16M_P90W_V93I_R97A | 1.000 | 1.260 | 0.060 | 1.710 | 0.000 | 3.000 |
| 1881 | Y7C_I16M_P90W_V93I_R97F | 0.000 | 2.730 | 1.000 | 1.390 | 1.000 | −0.820 |
| 1882 | Y7C_I16M_P90W_V93I_R97H | 0.000 | 2.390 | 0.000 | 2.450 | 1.000 | −0.680 |
| 1883 | Y7C_I16M_P90W_V93Q | 0.000 | 3.070 | 0.110 | 2.330 | 1.000 | 2.060 |
| 1884 | Y7C_I16M_P90Y | 0.000 | 3.010 | 1.000 | 1.820 | 0.070 | 2.360 |
| 1885 | Y7C_I16M_P90Y_V93I | 0.000 | 3.940 | 1.000 | 2.080 | 0.250 | 3.240 |
| 1886 | Y7C_I16M_P90Y_V93I_R97F | 0.000 | 2.590 | 0.050 | 1.160 | 1.000 | 0.420 |
| 1887 | Y7C_I16M_P90Y_V93R_R97A | 0.000 | 2.980 | 0.040 | 1.780 | 1.000 | 1.510 |
| 1888 | Y7C_I16M_V93I | 0.000 | 2.070 | 1.000 | 0.790 | 1.000 | 1.180 |
| 1889 | Y7C_I16M_V93I_R97F | 0.000 | 2.660 | 1.000 | −0.880 | 1.000 | 0.640 |
| 1890 | Y7C_I16M_V93R | 0.000 | 4.110 | 1.000 | 2.290 | 1.000 | 0.260 |
| 1891 | Y7C_I16W | 0.000 | 3.410 | 0.000 | 3.260 | 0.020 | 2.470 |
| 1892 | Y7C_I16W_P90F | 1.000 | 1.600 | 0.000 | 1.870 | 0.000 | 2.980 |
| 1893 | Y7C_I16W_P90F_R97A | 0.000 | 2.720 | 0.510 | 1.350 | 1.000 | 1.030 |
| 1894 | Y7C_I16W_P90I | 0.020 | 3.860 | 1.000 | −0.200 | 1.000 | −0.380 |
| 1895 | Y7C_I16W_V93I | 0.000 | 4.860 | 1.000 | 1.580 | 1.000 | 0.230 |
| 1896 | Y7C_P36A | 0.000 | 2.730 | 0.010 | 1.810 | 0.210 | 2.200 |
| 1897 | Y7C_P36A_P90F | 0.000 | 4.490 | 0.000 | 3.250 | 1.000 | 1.650 |
| 1898 | Y7C_P36A_P90F_V93I | 0.000 | 4.170 | 0.000 | 2.920 | 0.040 | 2.890 |
| 1899 | Y7C_P36A_P90F_V93I_R97H | 1.000 | 0.610 | 0.030 | 2.260 | 1.000 | 1.390 |
| 1900 | Y7C_P36A_P90F_V93Q | 0.000 | 3.160 | 1.000 | 2.090 | 1.000 | −1.600 |
| 1901 | Y7C_P36A_P90I | 0.010 | 3.450 | 1.000 | 0.110 | 1.000 | −0.990 |
| 1902 | Y7C_P36A_P90I_V93I | 0.000 | 2.940 | 1.000 | 0.480 | 1.000 | 0.120 |
| 1903 | Y7C_P36A_P90W | 0.000 | 5.290 | 0.000 | 4.090 | 0.000 | 3.990 |
| 1904 | Y7C_P36A_P90W_V93I | 0.000 | 4.920 | 0.000 | 5.130 | 0.010 | 3.270 |
| 1905 | Y7C_P36A_P90W_V93I_R97A | 0.000 | 2.800 | 0.000 | 2.580 | 1.000 | 1.690 |
| 1906 | Y7C_P36A_P90W_V93I_R97H | 0.000 | 3.180 | 1.000 | 0.190 | 1.000 | −0.200 |
| 1907 | Y7C_P36A_P90W_V93R_R97A | 0.000 | 2.300 | 1.000 | 1.180 | 1.000 | −1.380 |
| 1908 | Y7C_P36A_P90Y | 0.000 | 2.000 | 0.910 | 2.850 | 0.970 | 1.800 |
| 1909 | Y7C_P36A_P90Y_V93I | 0.000 | 4.770 | 0.000 | 3.640 | 0.000 | 4.060 |
| 1910 | Y7C_P36A_P90Y_V93I_R97A | 0.020 | 3.660 | 1.000 | −0.530 | 1.000 | 0.880 |
| 1911 | Y7C_P36A_P90Y_V93I_R97W | 0.000 | 2.220 | 1.000 | −0.480 | 1.000 | −2.500 |
| 1912 | Y7C_P36A_V93I | 0.000 | 2.580 | 1.000 | 0.100 | 1.000 | 0.930 |
| 1913 | Y7C_P36H | 0.070 | 2.510 | 0.000 | 1.750 | 0.000 | 2.990 |
| 1914 | Y7C_P36H_P90F | 0.390 | 2.630 | 0.000 | 2.510 | 1.000 | 0.790 |
| 1915 | Y7C_P36H_P90F_R97H | 0.010 | 2.160 | 1.000 | 0.790 | 1.000 | −1.230 |
| 1916 | Y7C_P36H_P90F_V93I | 0.150 | 3.010 | 0.000 | 3.100 | 1.000 | 2.310 |
| 1917 | Y7C_P36H_P90F_V93I_R97H | 0.000 | 2.560 | 0.010 | 2.090 | 1.000 | −1.180 |
| 1918 | Y7C_P36H_P90W | 0.160 | 3.460 | 0.000 | 3.010 | 1.000 | 1.290 |
| 1919 | Y7C_P36H_P90W_R97H | 0.010 | 2.180 | 1.000 | 0.390 | 1.000 | −2.330 |
| 1920 | Y7C_P36H_P90W_V93I | 0.380 | 2.730 | 0.000 | 3.270 | 1.000 | 1.310 |
| 1921 | Y7C_P36H_V93I | 0.000 | 3.760 | 1.000 | 1.240 | 0.000 | 1.500 |
| 1922 | Y7C_P36R_R97A | 0.000 | 2.240 | 1.000 | 0.870 | 1.000 | −0.250 |
| 1923 | Y7C_P36R_V93I_R97A | 0.000 | 2.080 | 1.000 | 1.170 | 1.000 | 1.120 |
| 1924 | Y7C_P36S_P90F_R97F | 0.000 | 2.250 | 1.000 | −0.740 | 1.000 | 0.440 |
| 1925 | Y7C_P36S_P90F_V93I | 0.000 | 3.670 | 0.050 | 2.830 | 0.310 | 3.210 |
| 1926 | Y7C_P36S_P90F_V93I_R97A | 0.000 | 2.800 | 0.000 | 1.960 | 0.020 | 2.030 |
| 1927 | Y7C_P36S_P90I | 0.000 | 2.740 | 1.000 | 1.110 | 0.000 | 2.450 |
| 1928 | Y7C_P36S_P90W | 0.000 | 4.560 | 0.570 | 3.040 | 1.000 | 1.100 |
| 1929 | Y7C_P36S_P90W_V93I | 0.000 | 3.270 | 1.000 | 0.950 | 1.000 | 0.740 |
| 1930 | Y7C_P36S_P90W_V93Q | 0.040 | 2.400 | 1.000 | 0.930 | 1.000 | −0.620 |
| 1931 | Y7C_P36S_P90Y_R97A | 0.840 | 2.730 | 1.000 | 1.690 | 0.000 | 2.930 |
| 1932 | Y7C_P36S_P90Y_V93I | 0.430 | 2.730 | 0.000 | 3.280 | 1.000 | 1.750 |
| 1933 | Y7C_P36W | 0.000 | 4.900 | 0.000 | 3.610 | 0.000 | 4.060 |
| 1934 | Y7C_P36W_P90F | 0.000 | 3.360 | 0.000 | 2.940 | 1.000 | 1.720 |
| 1935 | Y7C_P36W_P90F_V93I | 0.000 | 5.280 | 0.000 | 4.800 | 0.000 | 4.740 |
| 1936 | Y7C_P36W_P90F_V93I_R97A | 1.000 | 2.120 | 0.000 | 3.010 | 1.000 | 2.620 |
| 1937 | Y7C_P36W_P90F_V93I_R97H | 0.000 | 3.770 | 0.020 | 1.950 | 1.000 | 1.450 |
| 1938 | Y7C_P36W_P90W | 0.000 | 5.140 | 0.000 | 5.270 | 0.050 | 3.240 |
| 1939 | Y7C_P36W_P90W_R97A | 0.000 | 2.480 | 0.000 | 2.880 | 1.000 | −1.730 |
| 1940 | Y7C_P36W_P90W_R97H | 0.000 | 3.640 | 1.000 | 1.950 | 0.120 | 1.790 |
| 1941 | Y7C_P36W_P90W_V93I | 0.000 | 5.840 | 0.000 | 5.810 | 0.000 | 3.760 |
| 1942 | Y7C_P36W_P90W_V93I_R97A | 0.000 | 3.360 | 0.000 | 2.870 | 1.000 | 0.390 |
| 1943 | Y7C_P36W_P90W_V93I_R97W | 0.000 | 2.030 | 0.000 | 2.590 | 0.640 | 2.220 |
| 1944 | Y7C_P36W_P90W_V93R | 0.000 | 2.390 | 1.000 | −0.770 | 1.000 | 1.040 |
| 1945 | Y7C_P36W_P90Y | 0.000 | 3.970 | 0.000 | 3.810 | 1.000 | 1.900 |
| 1946 | Y7C_P36W_P90Y_V93I | 0.000 | 5.340 | 0.000 | 5.380 | 0.000 | 4.320 |
| 1947 | Y7C_P36W_P90Y_V93I_R97F | 0.010 | 2.610 | 1.000 | −0.120 | 1.000 | 0.180 |
| 1948 | Y7C_P36W_P90Y_V93I_R97H | 0.000 | 2.210 | 0.190 | 1.490 | 1.000 | 1.150 |
| 1949 | Y7C_P36W_P90Y_V93R | 0.000 | 2.280 | 1.000 | 0.440 | 1.000 | 0.620 |
| 1950 | Y7C_P36W_R97A | 1.000 | 0.560 | 0.000 | 2.760 | 1.000 | 1.340 |
| 1951 | Y7C_P36W_V93I | 0.000 | 5.300 | 0.000 | 3.850 | 0.000 | 4.210 |
| 1952 | Y7C_P36W_V93I_R97F | 0.000 | 2.440 | 1.000 | 1.930 | 1.000 | 0.210 |
| 1953 | Y7C_P90F | 0.000 | 4.080 | 0.000 | 2.890 | 0.090 | 2.080 |
| 1954 | Y7C_P90F_R97H | 0.000 | 2.000 | 1.000 | 1.180 | 1.000 | 1.270 |
| 1955 | Y7C_P90F_V93I | 0.000 | 5.010 | 0.000 | 3.120 | 0.000 | 4.310 |
| 1956 | Y7C_P90F_V93I_R97A | 0.000 | 2.460 | 0.000 | 2.570 | 1.000 | 2.130 |
| 1957 | Y7C_P90F_V93I_R97F | 1.000 | 1.460 | 0.000 | 2.970 | 1.000 | −0.030 |
| 1958 | Y7C_P90F_V93I_R97W | 1.000 | 1.990 | 0.010 | 2.770 | 1.000 | −1.160 |
| 1959 | Y7C_P90F_V93K | 1.000 | 1.480 | 0.000 | 2.660 | 1.000 | 1.690 |
| 1960 | Y7C_P90F_V93Q | 0.000 | 3.450 | 1.000 | 1.600 | 1.000 | −0.470 |
| 1961 | Y7C_P90F_V93Q_R97A | 0.000 | 2.220 | 0.020 | 2.480 | 1.000 | −0.090 |
| 1962 | Y7C_P90I | 0.000 | 4.700 | 1.000 | 1.210 | 1.000 | 0.580 |
| 1963 | Y7C_P90I_V93I | 0.000 | 4.380 | 0.000 | 1.060 | 1.000 | 1.770 |
| 1964 | Y7C_P90I_V93I_R97A | 0.000 | 3.270 | 0.000 | 3.090 | 0.000 | 3.270 |
| 1965 | Y7C_P90I_V93I_R97F | 0.000 | 2.630 | 1.000 | −0.050 | 1.000 | 0.540 |
| 1966 | Y7C_P90I_V93I_R97W | 0.000 | 2.150 | 0.680 | 3.050 | 0.660 | 1.500 |
| 1967 | Y7C_P90S_V93R_R97A | 0.000 | 3.640 | 1.000 | 0.170 | 1.000 | −2.290 |
| 1968 | Y7C_P90W | 0.000 | 4.240 | 0.000 | 4.320 | 0.000 | 4.500 |
| 1969 | Y7C_P90W_R97F | 1.000 | 1.730 | 0.000 | 3.010 | 1.000 | −1.320 |
| 1970 | Y7C_P90W_R97W | 1.000 | 1.180 | 0.030 | 2.490 | 1.000 | 0.230 |
| 1971 | Y7C_P90W_V93E | 1.000 | 0.220 | 0.000 | 2.320 | 1.000 | −0.500 |
| 1972 | Y7C_P90W_V93I | 0.000 | 4.350 | 0.000 | 4.060 | 0.000 | 3.980 |
| 1973 | Y7C_P90W_V93I_R97A | 1.000 | 1.570 | 0.000 | 3.160 | 1.000 | 1.930 |
| 1974 | Y7C_P90W_V93I_R97W | 0.000 | 3.040 | 1.000 | 2.050 | 1.000 | 0.220 |
| 1975 | Y7C_P90W_V93I_R119Q | 0.000 | 2.420 | 1.000 | −1.160 | 1.000 | −0.760 |
| 1976 | Y7C_P90W_V93K | 0.000 | 2.780 | 0.110 | 1.970 | 1.000 | 0.310 |
| 1977 | Y7C_P90W_V93Q | 0.000 | 4.580 | 0.000 | 4.190 | 1.000 | 1.290 |
| 1978 | Y7C_P90W_V93R | 0.000 | 3.690 | 0.000 | 3.600 | 1.000 | 2.290 |
| 1979 | Y7C_P90W_V93R_R97F | 1.000 | 0.000 | 0.000 | 2.120 | 1.000 | −2.570 |
| 1980 | Y7C_P90W_V93R_R119Q | 0.020 | 2.090 | 0.080 | 1.950 | 1.000 | −4.230 |
| 1981 | Y7C_P90Y | 0.000 | 4.060 | 0.090 | 1.820 | 0.040 | 2.750 |
| 1982 | Y7C_P90Y_R97H | 0.000 | 3.570 | 1.000 | 1.580 | 1.000 | 0.300 |
| 1983 | Y7C_P90Y_V93I | 0.000 | 3.690 | 0.000 | 3.080 | 0.000 | 3.470 |
| 1984 | Y7C_P90Y_V93I_R97F | 0.000 | 3.180 | 0.520 | 2.020 | 1.000 | −0.680 |
| 1985 | Y7C_P90Y_V93I_R97H | 0.000 | 3.060 | 1.000 | 1.920 | 1.000 | 2.020 |
| 1986 | Y7C_P90Y_V93K | 0.000 | 3.770 | 1.000 | 0.120 | 1.000 | −1.750 |
| 1987 | Y7C_P90Y_V93Q | 1.000 | 1.530 | 0.000 | 3.270 | 1.000 | −0.590 |
| 1988 | Y7C_V93I_R97W | 0.000 | 4.610 | 0.000 | 4.090 | 0.000 | 4.200 |
| 1989 | Y7C_V93K | 0.000 | 3.070 | 1.000 | 2.530 | 1.000 | −0.380 |
| 1990 | Y7C_V93R | 0.000 | 3.890 | 0.010 | 2.740 | 1.000 | 1.200 |
| 1991 | Y7D_I16L | 0.000 | 3.670 | 1.000 | −0.110 | 1.000 | 0.590 |
| 1992 | Y7D_I16L_P90Y | 1.000 | 1.430 | 1.000 | 1.670 | 0.010 | 3.950 |
| 1993 | Y7D_I16L_P90Y_V93I | 0.000 | 4.500 | 0.000 | 4.100 | 0.000 | 4.970 |
| 1994 | Y7D_P36A_P90F_V93I_R97A_R119Q | 1.000 | 0.820 | 1.000 | −0.460 | 0.030 | 2.970 |
| 1995 | Y7D_P36A_P90W_V93I | 0.000 | 3.370 | 0.060 | 3.290 | 1.000 | 2.000 |
| 1996 | Y7D_P36A_P90Y_V93I | 0.000 | 2.700 | 0.000 | 3.160 | 1.000 | 1.220 |
| 1997 | Y7D_P36A_V93I_R97A | 0.000 | 2.040 | 1.000 | 0.900 | 0.000 | 1.800 |
| 1998 | Y7D_P90F_V93I | 0.310 | 2.430 | 0.000 | 3.110 | 0.480 | 2.540 |
| 1999 | Y7D_P90I_V93I_R97F | 0.000 | 4.080 | 0.000 | 2.900 | 0.000 | 3.330 |
| 2000 | Y7D_P90S_V93I | 0.000 | 2.580 | 1.000 | −0.200 | 1.000 | −1.880 |
| 2001 | Y7D_P90W | 0.000 | 3.470 | 0.000 | 4.510 | 1.000 | 0.690 |
| 2002 | Y7D_P90W_V93I | 0.000 | 4.790 | 0.000 | 4.500 | 0.490 | 2.500 |
| 2003 | Y7D_P90Y | 0.030 | 3.160 | 0.000 | 3.030 | 0.000 | 2.420 |
| 2004 | Y7D_P90Y_V93I | 0.070 | 3.410 | 0.000 | 4.550 | 0.000 | 4.390 |
| 2005 | Y7D_P90Y_V93K | 0.000 | 3.010 | 1.000 | −1.030 | 1.000 | −0.370 |
| 2006 | Y7D_V93I_R97F | 0.000 | 5.910 | 0.000 | 7.600 | 0.000 | 6.870 |
| 41 | Y7L | 0.000 | 4.365 | 0.000 | 3.529 | 0.000 | 3.782 |
| 2007 | Y7L_I16F | 0.000 | 7.730 | 0.000 | 4.840 | 0.000 | 5.370 |
| 2008 | Y7L_I16F_P36A | 0.000 | 5.680 | 0.490 | 1.730 | 0.070 | 1.450 |
| 2009 | Y7L_I16F_P36A_P90F | 0.000 | 5.790 | 0.000 | 4.400 | 0.000 | 5.660 |
| 2010 | Y7L_I16F_P36A_P90F_R97A | 1.000 | 0.430 | 0.000 | 4.160 | 1.000 | 1.860 |
| 2011 | Y7L_I16F_P36A_P90F_V93I | 0.000 | 8.240 | 0.000 | 6.330 | 0.000 | 7.320 |
| 2012 | Y7L_I16F_P36A_P90F_V93I_R97A | 0.000 | 4.730 | 0.020 | 4.240 | 0.000 | 4.430 |
| 2013 | Y7L_I16F_P36A_P90F_V93I_R97F | 0.000 | 3.200 | 0.000 | 3.840 | 1.000 | 2.120 |
| 2014 | Y7L_I16F_P36A_P90F_V93I_R97H | 0.000 | 3.940 | 0.060 | 3.800 | 1.000 | 2.870 |
| 2015 | Y7L_I16F_P36A_P90I | 0.000 | 4.390 | 0.100 | 3.390 | 0.670 | 1.900 |
| 2016 | Y7L_I16F_P36A_P90I_R97H | 0.000 | 2.860 | 0.130 | 1.750 | 0.000 | 2.100 |
| 2017 | Y7L_I16F_P36A_P90I_V93I | 0.000 | 4.270 | 1.000 | 3.010 | 0.000 | 3.310 |
| 2018 | Y7L_I16F_P36A_P90W | 0.000 | 5.440 | 0.000 | 3.500 | 0.000 | 4.190 |
| 2019 | Y7L_I16F_P36A_P90W_V93I | 0.000 | 5.090 | 0.000 | 5.000 | 0.000 | 5.210 |
| 2020 | Y7L_I16F_P36A_P90W_V93I_R97F | 0.000 | 3.180 | 1.000 | 1.300 | 1.000 | 0.740 |
| 2021 | Y7L_I16F_P36A_P90W_V93I_R97H | 0.010 | 3.180 | 1.000 | 1.260 | 1.000 | 1.180 |
| 2022 | Y7L_I16F_P36A_P90Y | 0.010 | 3.890 | 0.010 | 4.320 | 0.020 | 4.350 |
| 2023 | Y7L_I16F_P36A_P90Y_R97A | 0.020 | 3.710 | 1.000 | 1.670 | 1.000 | 1.130 |
| 2024 | Y7L_I16F_P36A_P90Y_V93I | 0.000 | 6.490 | 0.000 | 5.620 | 0.000 | 6.210 |
| 2025 | Y7L_I16F_P36A_P90Y_V93I_R97A | 0.000 | 5.440 | 0.000 | 5.440 | 0.000 | 3.840 |
| 2026 | Y7L_I16F_P36A_P90Y_V93I_R97F | 0.390 | 2.090 | 0.000 | 2.610 | 1.000 | 1.220 |
| 2027 | Y7L_I16F_P36A_R97A | 0.000 | 5.800 | 0.000 | 5.410 | 0.000 | 5.290 |
| 2028 | Y7L_I16F_P36A_R97F | 0.400 | 3.130 | 0.000 | 3.050 | 1.000 | 1.370 |
| 2029 | Y7L_I16F_P36A_V93I | 0.000 | 8.910 | 0.760 | 2.270 | 0.120 | 1.460 |
| 2030 | Y7L_I16F_P36A_V93I_R97A | 0.000 | 5.210 | 0.190 | 2.540 | 0.020 | 2.690 |
| 2031 | Y7L_I16F_P36A_V93I_R97F | 0.000 | 5.080 | 0.000 | 5.230 | 0.000 | 3.670 |
| 2032 | Y7L_I16F_P36A_V93I_R97H | 0.000 | 4.110 | 0.000 | 3.530 | 0.000 | 5.030 |
| 2033 | Y7L_I16F_P36A_V93I_R97W | 0.000 | 5.380 | 0.010 | 4.800 | 0.000 | 5.490 |
| 2034 | Y7L_I16F_P36A_V93Q_R97A | 1.000 | 1.630 | 0.000 | 4.070 | 1.000 | 2.890 |
| 2035 | Y7L_I16F_P36A_V93Q_R97F | 0.000 | 3.220 | 0.000 | 3.370 | 0.000 | 4.270 |
| 2036 | Y7L_I16F_P36A_V93R | 0.000 | 2.450 | 1.000 | 2.450 | 0.000 | 3.020 |
| 2037 | Y7L_I16F_P36A_V93R_R97A | 0.040 | 4.130 | 0.130 | 4.700 | 1.000 | 1.580 |
| 2038 | Y7L_I16F_P36H | 0.000 | 4.730 | 0.000 | 5.300 | 0.000 | 3.050 |
| 2039 | Y7L_I16F_P36H_R97W | 0.000 | 3.020 | 0.000 | 3.160 | 0.000 | 3.640 |
| 2040 | Y7L_I16F_P36H_V93I | 0.000 | 5.400 | 0.000 | 4.090 | 0.770 | 3.330 |
| 2041 | Y7L_I16F_P36H_V93R | 0.000 | 5.280 | 0.000 | 3.900 | 0.000 | 4.380 |
| 2042 | Y7L_I16F_P36R | 0.040 | 3.090 | 1.000 | 2.380 | 1.000 | −0.970 |
| 2043 | Y7L_I16F_P36R_R97A | 0.000 | 4.630 | 1.000 | 1.040 | 1.000 | 1.670 |
| 2044 | Y7L_I16F_P36S | 0.000 | 5.140 | 1.000 | 0.310 | 0.800 | 1.580 |
| 2045 | Y7L_I16F_P36S_P90F_V93I | 0.000 | 3.490 | 1.000 | 2.070 | 1.000 | 2.860 |
| 2046 | Y7L_I16F_P36S_P90F_V93I_R97A | 1.000 | 1.720 | 0.000 | 2.510 | 1.000 | −0.790 |
| 2047 | Y7L_I16F_P36S_R97H | 0.000 | 4.280 | 0.000 | 3.210 | 1.000 | 1.410 |
| 2048 | Y7L_I16F_P36S_V93I | 0.000 | 6.810 | 1.000 | 1.040 | 1.000 | −0.130 |
| 2049 | Y7L_I16F_P36S_V93I_R97A | 0.000 | 4.470 | 0.000 | 4.470 | 0.000 | 4.690 |
| 2050 | Y7L_I16F_P36S_V93I_R97H | 0.000 | 3.420 | 1.000 | 2.450 | 1.000 | 1.220 |
| 2051 | Y7L_I16F_P36W | 0.000 | 7.180 | 0.000 | 7.170 | 0.000 | 7.520 |
| 2052 | Y7L_I16F_P36W_P90F | 0.000 | 5.420 | 0.000 | 4.900 | 0.000 | 4.930 |
| 2053 | Y7L_I16F_P36W_P90F_V93I | 0.000 | 5.770 | 0.000 | 6.050 | 0.000 | 4.770 |
| 2054 | Y7L I16F P36W P90F V93I R97A | 0.000 | 4.400 | 0.000 | 4.930 | 0.350 | 2.540 |
| 2055 | Y7L_I16F_P36W_P90F_V93I_R97H | 0.000 | 3.030 | 0.000 | 3.790 | 0.040 | 2.520 |
| 2056 | Y7L_I16F_P36W_P90I_V93I | 0.000 | 3.240 | 1.000 | 0.180 | 0.490 | 1.890 |
| 2057 | Y7L_I16F_P36W_P90W | 0.320 | 3.170 | 0.000 | 3.940 | 1.000 | 1.760 |
| 2058 | Y7L_I16F_P36W_P90W_R97A | 1.000 | 1.400 | 0.000 | 2.870 | 1.000 | 1.160 |
| 2059 | Y7L_I16F_P36W_P90W_R97H | 0.000 | 2.590 | 1.000 | −0.310 | 1.000 | −1.170 |
| 2060 | Y7L_I16F_P36W_P90W_R97W | 0.000 | 3.470 | 0.000 | 2.170 | 0.000 | 3.040 |
| 2061 | Y7L_I16F_P36W_P90W_V93I | 0.000 | 6.060 | 0.000 | 5.330 | 0.000 | 3.500 |
| 2062 | Y7L_I16F_P36W_P90W_V93I_R97F | 0.000 | 4.530 | 0.450 | 4.200 | 1.000 | 1.530 |
| 2063 | Y7L_I16F_P36W_P90Y_R97A | 0.000 | 3.980 | 1.000 | 1.200 | 1.000 | 2.320 |
| 2064 | Y7L_I16F_P36W_P90Y_V93I | 0.000 | 5.740 | 0.000 | 3.530 | 0.010 | 4.240 |
| 2065 | Y7L_I16F_P36W_P90Y_V93I_R97A | 0.160 | 3.130 | 0.000 | 4.830 | 1.000 | 2.140 |
| 2066 | Y7L_I16F_P36W_P90Y_V93I_R97F | 0.000 | 3.400 | 0.000 | 2.790 | 1.000 | 0.890 |
| 2067 | Y7L_I16F_P36W_P90Y_V93I_R97H | 0.390 | 2.760 | 0.010 | 3.770 | 1.000 | 1.390 |
| 2068 | Y7L_I16F_P36W_R97F | 0.000 | 2.330 | 0.000 | 3.260 | 1.000 | 1.310 |
| 2069 | Y7L_I16F_P36W_R97H | 0.000 | 5.260 | 1.000 | 2.770 | 0.030 | 3.110 |
| 2070 | Y7L_I16F_P36W_V93I | 0.000 | 8.020 | 0.000 | 5.310 | 0.000 | 7.690 |
| 2071 | Y7L_I16F_P36W_V93I_R97A | 0.000 | 4.950 | 0.000 | 5.710 | 0.040 | 3.650 |
| 2072 | Y7L_I16F_P36W_V93I_R97F | 0.010 | 3.260 | 0.000 | 4.100 | 0.000 | 3.250 |
| 2073 | Y7L_I16F_P36W_V93I_R97H | 0.010 | 4.670 | 0.000 | 4.560 | 1.000 | 1.830 |
| 2074 | Y7L_I16F_P36W_V93I_R97W | 0.000 | 4.470 | 0.320 | 2.820 | 0.000 | 3.660 |
| 2075 | Y7L_I16F_P90F | 0.000 | 10.380 | 0.000 | 9.000 | 0.000 | 10.780 |
| 2076 | Y7L_I16F_P90F_R97A | 0.000 | 8.070 | 0.000 | 6.440 | 0.000 | 5.930 |
| 2077 | Y7L_I16F_P90F_R97F | 0.000 | 7.460 | 0.000 | 7.410 | 0.000 | 5.900 |
| 2078 | Y7L_I16F_P90F_R97H | 0.000 | 7.410 | 0.000 | 6.850 | 0.000 | 5.280 |
| 2079 | Y7L_I16F_P90F_R97W | 0.000 | 6.740 | 0.000 | 5.580 | 0.000 | 4.170 |
| 2080 | Y7L_I16F_P90F_V93I | 0.000 | 11.040 | 0.000 | 10.200 | 0.000 | 11.220 |
| 2081 | Y7L_I16F_P90F_V93I_R97A | 0.000 | 7.840 | 0.000 | 7.710 | 0.000 | 6.410 |
| 2082 | Y7L_I16F_P90F_V93I_R97F | 0.000 | 8.110 | 0.000 | 8.140 | 0.000 | 7.280 |
| 2083 | Y7L_I16F_P90F_V93I_R97H | 0.000 | 8.150 | 0.000 | 7.210 | 0.000 | 6.870 |
| 2084 | Y7L_I16F_P90F_V93I_R97W | 0.000 | 6.660 | 0.000 | 7.270 | 0.000 | 5.070 |
| 2085 | Y7L_I16F_P90F_V93K | 0.000 | 5.680 | 0.000 | 3.740 | 0.000 | 4.100 |
| 2086 | Y7L_I16F_P90F_V93K_R97A | 0.000 | 3.920 | 1.000 | 1.230 | 1.000 | 0.900 |
| 2087 | Y7L_I16F_P90F_V93K_R97F | 0.020 | 2.360 | 0.150 | 2.240 | 1.000 | 0.880 |
| 2088 | Y7L_I16F_P90F_V93Q | 0.000 | 6.340 | 0.000 | 5.820 | 0.000 | 4.740 |
| 2089 | Y7L_I16F_P90F_V93Q_R97A | 0.000 | 5.960 | 0.000 | 5.050 | 0.000 | 4.640 |
| 2090 | Y7L_I16F_P90F_V93Q_R97F | 1.000 | 1.410 | 0.000 | 2.570 | 0.960 | 2.180 |
| 2091 | Y7L_I16F_P90F_V93Q_R97W | 0.030 | 2.720 | 0.040 | 2.690 | 0.030 | 2.900 |
| 2092 | Y7L_I16F_P90F_V93R | 0.050 | 3.260 | 0.280 | 3.600 | 0.000 | 4.100 |
| 2093 | Y7L_I16F_P90F_V93R_R97A | 0.000 | 8.120 | 0.000 | 6.870 | 0.000 | 4.980 |
| 2094 | Y7L_I16F_P90F_V93R_R97F | 0.000 | 4.260 | 0.000 | 2.760 | 0.000 | 3.530 |
| 2095 | Y7L_I16F_P90I | 0.000 | 11.830 | 0.000 | 8.850 | 0.000 | 10.100 |
| 2096 | Y7L_I16F_P90I_R97A | 0.000 | 6.760 | 0.000 | 6.360 | 0.000 | 6.300 |
| 2097 | Y7L_I16F_P90I_R97F | 0.000 | 7.170 | 0.000 | 6.540 | 0.000 | 6.780 |
| 2098 | Y7L_I16F_P90I_R97H | 0.000 | 4.170 | 0.000 | 4.820 | 0.470 | 3.150 |
| 2099 | Y7L_I16F_P90I_R97W | 0.000 | 5.490 | 0.000 | 6.040 | 0.000 | 5.200 |
| 2100 | Y7L_I16F_P90I_V93I | 0.000 | 11.720 | 0.000 | 11.120 | 0.000 | 13.270 |
| 2101 | Y7L_I16F_P90I_V93I_R97A | 0.000 | 6.750 | 0.000 | 7.910 | 0.000 | 6.200 |
| 2102 | Y7L_I16F_P90I_V93I_R97F | 0.000 | 8.410 | 0.000 | 6.670 | 0.000 | 5.810 |
| 2103 | Y7L_I16F_P90I_V93I_R97H | 0.000 | 8.400 | 0.000 | 7.370 | 0.000 | 5.730 |
| 2104 | Y7L_I16F_P90I_V93I_R97W | 0.000 | 4.230 | 0.000 | 5.880 | 0.000 | 3.400 |
| 2105 | Y7L_I16F_P90I_V93I_R119Q | 1.000 | 0.490 | 0.260 | 1.090 | 0.000 | 3.020 |
| 2106 | Y7L_I16F_P90I_V93K | 0.000 | 2.570 | 1.000 | 1.960 | 1.000 | 0.530 |
| 2107 | Y7L_I16F_P90I_V93K_R97A | 0.220 | 3.120 | 0.000 | 2.500 | 1.000 | 0.850 |
| 2108 | Y7L_I16F_P90I_V93K_R97W | 0.000 | 3.970 | 0.000 | 2.920 | 0.000 | 4.190 |
| 2109 | Y7L_I16F_P90I_V93Q | 0.000 | 6.580 | 0.000 | 6.040 | 0.000 | 4.900 |
| 2110 | Y7L_I16F_P90I_V93Q_R97F | 1.000 | 0.860 | 0.000 | 2.720 | 1.000 | 1.880 |
| 2111 | Y7L_I16F_P90I_V93R | 0.000 | 7.640 | 0.000 | 7.240 | 0.000 | 6.840 |
| 2112 | Y7L_I16F_P90I_V93R_R97A | 0.000 | 6.360 | 0.000 | 5.220 | 0.000 | 4.120 |
| 2113 | Y7L_I16F_P90I_V93R_R97W | 0.000 | 3.670 | 0.000 | 2.970 | 0.000 | 3.530 |
| 2114 | Y7L_I16F_P90S | 0.000 | 6.330 | 1.000 | −0.250 | 1.000 | 1.520 |
| 2115 | Y7L_I16F_P90S_R97A | 0.000 | 6.030 | 0.000 | 6.440 | 0.000 | 6.060 |
| 2116 | Y7L_I16F_P90S_R97F | 0.690 | 2.890 | 0.340 | 3.520 | 0.010 | 3.170 |
| 2117 | Y7L_I16F_P90S_V93I | 0.000 | 7.000 | 1.000 | 0.690 | 0.000 | 2.480 |
| 2118 | Y7L_I16F_P90S_V93I_R97A | 0.000 | 6.690 | 0.000 | 5.510 | 0.000 | 6.550 |
| 2119 | Y7L_I16F_P90S_V93I_R97F | 0.010 | 2.620 | 0.030 | 3.640 | 0.000 | 3.240 |
| 2120 | Y7L_I16F_P90S_V93I_R97H | 0.000 | 5.150 | 0.000 | 5.360 | 0.000 | 5.410 |
| 2121 | Y7L_I16F_P90S_V93I_R97W | 0.210 | 1.970 | 1.000 | 1.020 | 0.020 | 2.750 |
| 2122 | Y7L_I16F_P90S_V93K | 0.000 | 3.280 | 0.040 | 2.340 | 1.000 | 0.650 |
| 2123 | Y7L_I16F_P90S_V93K_R97A | 0.000 | 3.450 | 0.680 | 2.010 | 0.000 | 3.180 |
| 2124 | Y7L_I16F_P90S_V93Q | 0.000 | 3.600 | 0.030 | 2.460 | 0.000 | 3.680 |
| 2125 | Y7L_I16F_P90S_V93Q_R97A | 0.000 | 4.520 | 0.000 | 3.730 | 0.000 | 4.770 |
| 2126 | Y7L_I16F_P90S_V93R_R97F | 0.000 | 2.940 | 1.000 | 0.950 | 1.000 | 1.090 |
| 2127 | Y7L_I16F_P90W | 0.000 | 10.710 | 0.000 | 8.450 | 0.000 | 10.600 |
| 2128 | Y7L_I16F_P90W_R97A | 0.000 | 9.130 | 0.000 | 7.100 | 0.000 | 5.510 |
| 2129 | Y7L_I16F_P90W_R97F | 0.000 | 8.030 | 0.000 | 6.570 | 0.000 | 4.950 |
| 2130 | Y7L_I16F_P90W_R97H | 0.000 | 7.320 | 0.000 | 7.140 | 0.000 | 5.680 |
| 2131 | Y7L_I16F_P90W_R97W | 0.000 | 7.190 | 0.000 | 6.530 | 0.000 | 5.090 |
| 2132 | Y7L_I16F_P90W_V93I | 0.000 | 10.520 | 0.000 | 9.210 | 0.000 | 11.230 |
| 2133 | Y7L_I16F_P90W_V93I_R97A | 0.000 | 8.670 | 0.000 | 8.140 | 0.000 | 7.030 |
| 2134 | Y7L_I16F_P90W_V93I_R97F | 0.000 | 7.960 | 0.000 | 7.800 | 0.000 | 6.500 |
| 2135 | Y7L_I16F_P90W_V93I_R97H | 0.000 | 8.420 | 0.000 | 7.310 | 0.000 | 6.280 |
| 2136 | Y7L_I16F_P90W_V93I_R97W | 0.000 | 6.760 | 0.000 | 6.170 | 0.000 | 5.930 |
| 2137 | Y7L_I16F_P90W_V93K_R97W | 0.280 | 3.080 | 0.000 | 1.860 | 0.000 | 2.450 |
| 2138 | Y7L_I16F_P90W_V93Q | 0.000 | 6.100 | 0.000 | 5.880 | 0.000 | 3.660 |
| 2139 | Y7L_I16F_P90W_V93Q_R97A | 0.000 | 4.530 | 0.020 | 3.980 | 0.010 | 3.940 |
| 2140 | Y7L_I16F_P90W_V93Q_R97W | 0.000 | 5.340 | 0.000 | 4.300 | 0.000 | 5.340 |
| 2141 | Y7L_I16F_P90W_V93R | 0.000 | 3.720 | 0.000 | 5.430 | 0.000 | 3.150 |
| 2142 | Y7L_I16F_P90W_V93R_R97A | 0.000 | 6.250 | 0.000 | 6.880 | 0.000 | 5.130 |
| 2143 | Y7L_I16F_P90W_V93R_R97F | 0.000 | 5.720 | 0.000 | 4.680 | 0.020 | 3.320 |
| 2144 | Y7L_I16F_P90W_V93R_R97W | 0.000 | 6.360 | 0.270 | 3.980 | 0.430 | 1.630 |
| 2145 | Y7L_I16F_P90Y | 0.000 | 6.710 | 0.000 | 5.910 | 0.000 | 6.740 |
| 2146 | Y7L_I16F_P90Y_R97A | 0.000 | 7.250 | 0.000 | 7.280 | 0.000 | 5.680 |
| 2147 | Y7L_I16F_P90Y_R97F | 0.000 | 6.510 | 0.000 | 6.970 | 0.000 | 5.990 |
| 2148 | Y7L_I16F_P90Y_R97H | 0.000 | 6.350 | 0.000 | 6.620 | 0.000 | 5.010 |
| 2149 | Y7L_I16F_P90Y_R97W | 0.000 | 5.770 | 0.000 | 5.600 | 0.000 | 4.320 |
| 2150 | Y7L_I16F_P90Y_V93I | 0.000 | 9.600 | 0.000 | 8.130 | 0.000 | 10.420 |
| 2151 | Y7L_I16F_P90Y_V93I_R97A | 0.000 | 7.500 | 0.000 | 7.660 | 0.000 | 6.580 |
| 2152 | Y7L_I16F_P90Y_V93I_R97F | 0.000 | 5.210 | 0.000 | 4.570 | 0.000 | 5.040 |
| 2153 | Y7L_I16F_P90Y_V93I_R97H | 0.000 | 7.220 | 0.000 | 7.010 | 0.000 | 6.260 |
| 2154 | Y7L_I16F_P90Y_V93I_R97W | 0.000 | 6.680 | 0.000 | 6.160 | 0.000 | 5.390 |
| 2155 | Y7L_I16F_P90Y_V93K_R97A | 0.000 | 5.410 | 0.000 | 4.670 | 0.000 | 5.730 |
| 2156 | Y7L_I16F_P90Y_V93K_R97P | 0.000 | 3.350 | 0.000 | 2.610 | 0.000 | 3.270 |
| 2157 | Y7L_I16F_P90Y_V93Q | 0.000 | 5.240 | 0.000 | 6.370 | 0.000 | 4.260 |
| 2158 | Y7L_I16F_P90Y_V93Q_R97A | 0.000 | 5.840 | 0.000 | 4.470 | 1.000 | 2.690 |
| 2159 | Y7L_I16F_P90Y_V93R_R97A | 0.000 | 7.120 | 0.000 | 4.500 | 0.000 | 3.670 |
| 2160 | Y7L_I16F_R97A | 0.000 | 7.960 | 0.000 | 7.320 | 0.000 | 7.450 |
| 2161 | Y7L_I16F_R97F | 0.000 | 7.710 | 0.000 | 7.820 | 0.000 | 6.830 |
| 2162 | Y7L_I16F_R97W | 0.000 | 6.690 | 0.000 | 6.220 | 0.000 | 6.620 |
| 2163 | Y7L_I16F_V93E_R97A | 0.010 | 3.590 | 0.580 | 2.670 | 1.000 | 2.160 |
| 2164 | Y7L_I16F_V93I | 0.000 | 11.800 | 0.000 | 10.110 | 0.000 | 11.010 |
| 2165 | Y7L_I16F_V93I_R97A | 0.000 | 8.050 | 0.000 | 7.400 | 0.000 | 8.440 |
| 2166 | Y7L_I16F_V93I_R97F | 0.000 | 8.020 | 0.000 | 6.480 | 0.000 | 6.860 |
| 2167 | Y7L_I16F_V93I_R97H | 0.000 | 6.780 | 0.000 | 7.020 | 0.000 | 8.170 |
| 2168 | Y7L_I16F_V93I_R97W | 0.000 | 7.510 | 0.000 | 6.930 | 0.000 | 8.180 |
| 2169 | Y7L_I16F_V93K | 0.000 | 4.750 | 0.000 | 4.960 | 0.000 | 3.800 |
| 2170 | Y7L_I16F_V93K_R97A | 0.000 | 5.590 | 0.000 | 5.700 | 0.000 | 5.790 |
| 2171 | Y7L_I16F_V93K_R97F | 0.000 | 4.980 | 0.010 | 3.320 | 0.800 | 2.080 |
| 2172 | Y7L_I16F_V93K_R97H | 0.200 | 2.760 | 0.000 | 3.410 | 1.000 | 2.350 |
| 2173 | Y7L_I16F_V93Q | 0.010 | 5.030 | 0.000 | 2.980 | 0.000 | 3.700 |
| 2174 | Y7L_I16F_V93Q_R97A | 0.000 | 5.920 | 0.000 | 5.740 | 0.000 | 4.040 |
| 2175 | Y7L_I16F_V93Q_R97F | 0.000 | 4.400 | 0.000 | 4.390 | 0.000 | 3.480 |
| 2176 | Y7L_I16F_V93Q_R97H | 0.000 | 5.130 | 0.000 | 4.610 | 0.000 | 4.060 |
| 2177 | Y7L_I16F_V93Q_R97W | 0.000 | 4.900 | 0.000 | 4.830 | 0.000 | 3.960 |
| 2178 | Y7L_I16F_V93R | 0.000 | 7.500 | 0.000 | 7.150 | 0.000 | 6.340 |
| 2179 | Y7L_I16F_V93R_R97A | 0.000 | 7.740 | 0.000 | 7.340 | 0.000 | 6.070 |
| 2180 | Y7L_I16F_V93R_R97A_R119Q | 0.060 | 3.680 | 0.000 | 5.380 | 0.370 | 2.890 |
| 2181 | Y7L_I16F_V93R_R97F | 0.000 | 5.880 | 0.010 | 5.450 | 0.000 | 4.760 |
| 2182 | Y7L_I16F_V93R_R97H | 0.000 | 5.140 | 0.000 | 5.170 | 0.000 | 4.380 |
| 2183 | Y7L_I16F_V93R_R97P | 0.000 | 3.820 | 0.180 | 3.260 | 0.000 | 4.070 |
| 2184 | Y7L_I16F_V93R_R97W | 0.050 | 3.460 | 0.010 | 4.290 | 1.000 | 2.260 |
| 2185 | Y7L_I16H_P36H_V93I | 0.000 | 7.290 | 0.000 | 5.420 | 0.050 | 3.140 |
| 2186 | Y7L_I16H_P36H_V93I_R97H | 1.000 | −1.330 | 0.000 | 2.570 | 1.000 | −1.740 |
| 2187 | Y7L_I16H_P36H_V93R | 0.000 | 4.520 | 0.010 | 3.790 | 1.000 | 0.790 |
| 2188 | Y7L_I16H_P90W_V93I | 0.000 | 3.100 | 0.000 | 2.880 | 1.000 | 2.750 |
| 2189 | Y7L_I16H_P90Y_V93I | 0.160 | 2.000 | 0.030 | 1.650 | 0.000 | 2.940 |
| 2190 | Y7L_I16L | 0.000 | 5.770 | 0.000 | 2.820 | 0.000 | 2.780 |
| 2191 | Y7L_I16L_P36A | 0.000 | 6.260 | 0.180 | 1.740 | 1.000 | 1.030 |
| 2192 | Y7L_I16L_P36A_P90F | 0.870 | 3.680 | 1.000 | 2.740 | 0.000 | 4.280 |
| 2193 | Y7L_I16L_P36A_P90F_V93I | 0.000 | 4.370 | 0.000 | 3.760 | 0.000 | 3.300 |
| 2194 | Y7L_I16L_P36A_P90F_V93I_R97A | 0.440 | 2.220 | 0.010 | 3.240 | 1.000 | 0.290 |
| 2195 | Y7L_I16L_P36A_P90F_V93I_R97H | 0.030 | 2.460 | 1.000 | 1.460 | 0.010 | 3.140 |
| 2196 | Y7L_I16L_P36A_P90F_V93Q_R97A | 0.000 | 3.090 | 1.000 | 1.270 | 1.000 | 0.890 |
| 2197 | Y7L_I16L_P36A_P90W | 0.000 | 3.910 | 1.000 | 1.030 | 1.000 | 1.170 |
| 2198 | Y7L_I16L_P36A_P90W_R97A | 0.000 | 2.410 | 1.000 | 1.700 | 1.000 | −0.250 |
| 2199 | Y7L_I16L_P36A_P90W_V93I | 0.020 | 3.990 | 0.000 | 4.500 | 0.250 | 2.430 |
| 2200 | Y7L_I16L_P36A_P90W_V93I_R97H | 0.000 | 2.820 | 1.000 | 0.690 | 1.000 | 0.220 |
| 2201 | Y7L_I16L_P36A_P90Y_V93I | 0.000 | 5.010 | 0.000 | 4.650 | 0.000 | 4.310 |
| 2202 | Y7L_I16L_P36A_R97A | 0.000 | 4.950 | 0.000 | 4.580 | 1.000 | 2.220 |
| 2203 | Y7L_I16L_P36A_R97F | 0.000 | 3.660 | 0.000 | 1.710 | 0.030 | 3.010 |
| 2204 | Y7L_I16L_P36A_R97H | 0.000 | 2.970 | 1.000 | 3.830 | 0.000 | 2.710 |
| 2205 | Y7L_I16L_P36A_R97W | 0.000 | 3.980 | 0.000 | 2.780 | 0.000 | 2.990 |
| 2206 | Y7L_I16L_P36A_V93I | 0.000 | 7.080 | 0.000 | 2.560 | 1.000 | 1.360 |
| 2207 | Y7L_I16L_P36A_V93I_R97A | 0.000 | 8.680 | 0.000 | 7.880 | 0.000 | 8.980 |
| 2208 | Y7L_I16L_P36A_V93I_R97F | 0.000 | 5.260 | 0.720 | 3.410 | 0.000 | 2.420 |
| 2209 | Y7L_I16L_P36A_V93I_R97H | 1.000 | 2.800 | 0.000 | 3.880 | 0.000 | 4.290 |
| 2210 | Y7L_I16L_P36A_V93Q | 0.000 | 4.750 | 0.000 | 3.320 | 1.000 | 1.980 |
| 2211 | Y7L_I16L_P36A_V93Q_R97A | 0.000 | 3.940 | 0.000 | 3.750 | 0.000 | 3.520 |
| 2212 | Y7L_I16L_P36A_V93R_R97A | 0.130 | 2.990 | 0.000 | 2.650 | 1.000 | 0.430 |
| 2213 | Y7L_I16L_P36H | 0.000 | 5.520 | 0.000 | 7.460 | 0.000 | 4.940 |
| 2214 | Y7L_I16L_P36H_P90I_V93I | 0.000 | 5.300 | 0.000 | 2.650 | 0.000 | 2.600 |
| 2215 | Y7L_I16L_P36H_P90W | 0.000 | 2.710 | 0.650 | 1.390 | 1.000 | 0.630 |
| 2216 | Y7L_I16L_P36H_P90Y_V93I | 0.000 | 2.290 | 1.000 | 0.770 | 1.000 | −0.300 |
| 2217 | Y7L_I16L_P36H_R97H | 0.000 | 3.790 | 0.000 | 4.520 | 0.000 | 3.930 |
| 2218 | Y7L_I16L_P36H_V93I | 0.000 | 7.110 | 0.000 | 6.820 | 0.000 | 5.390 |
| 2219 | Y7L_I16L_P36H_V93I_R97A | 1.000 | 1.260 | 0.000 | 2.730 | 1.000 | 0.190 |
| 2220 | Y7L_I16L_P36H_V93I_R97H | 0.000 | 4.510 | 0.000 | 4.250 | 0.000 | 3.920 |
| 2221 | Y7L_I16L_P36R | 0.000 | 3.640 | 0.020 | 3.120 | 1.000 | 2.000 |
| 2222 | Y7L_I16L_P36S | 0.000 | 3.900 | 0.000 | 2.950 | 0.000 | 3.080 |
| 2223 | Y7L_I16L_P36S_P90F_R97A | 0.000 | 2.100 | 1.000 | 0.340 | 1.000 | 0.420 |
| 2224 | Y7L_I16L_P36S_P90W_V93I_R97A | 0.010 | 2.060 | 1.000 | −1.410 | 1.000 | −3.300 |
| 2225 | Y7L_I16L_P36S_P90Y_V93I_R97A | 0.000 | 2.970 | 1.000 | 0.790 | 1.000 | 0.810 |
| 2226 | Y7L_I16L_P36S_V93I | 0.000 | 5.510 | 1.000 | −0.060 | 1.000 | −0.950 |
| 2227 | Y7L_I16L_P36S_V93I_R97A | 1.000 | 1.900 | 0.000 | 2.550 | 1.000 | 1.480 |
| 2228 | Y7L_I16L_P36S_V93I_R97F | 1.000 | 0.860 | 0.000 | 3.290 | 1.000 | 2.220 |
| 2229 | Y7L_I16L_P36W | 0.000 | 8.710 | 0.000 | 7.480 | 0.000 | 8.230 |
| 2230 | Y7L_I16L_P36W_P90F | 0.000 | 4.620 | 0.000 | 4.150 | 0.000 | 2.940 |
| 2231 | Y7L_I16L_P36W_P90F_R97A | 0.000 | 3.420 | 0.500 | 2.800 | 1.000 | 0.660 |
| 2232 | Y7L_I16L_P36W_P90F_V93I | 0.000 | 6.570 | 0.000 | 6.510 | 0.000 | 5.380 |
| 2233 | Y7L_I16L_P36W_P90F_V93I_R97A | 0.040 | 2.510 | 1.000 | 2.000 | 1.000 | 2.100 |
| 2234 | Y7L_I16L_P36W_P90F_V93I_R97H | 0.000 | 2.970 | 0.000 | 4.240 | 1.000 | 1.360 |
| 2235 | Y7L_I16L_P36W_P90I_V93I | 0.000 | 4.800 | 0.090 | 1.590 | 0.000 | 4.330 |
| 2236 | Y7L_I16L_P36W_P90I_V93I_R97A | 0.000 | 2.490 | 0.530 | 1.890 | 1.000 | −0.530 |
| 2237 | Y7L_I16L_P36W_P90W | 0.000 | 5.310 | 0.000 | 6.010 | 0.010 | 2.370 |
| 2238 | Y7L_I16L_P36W_P90W_R97H | 0.000 | 2.830 | 0.990 | 2.420 | 1.000 | −0.140 |
| 2239 | Y7L_I16L_P36W_P90W_V93I | 0.000 | 8.110 | 0.000 | 7.130 | 0.000 | 5.010 |
| 2240 | Y7L_I16L_P36W_P90W_V93I_R97H | 0.000 | 3.290 | 0.190 | 3.010 | 0.020 | 2.680 |
| 2241 | Y7L_I16L_P36W_P90Y | 0.000 | 4.620 | 0.000 | 4.880 | 0.000 | 2.940 |
| 2242 | Y7L_I16L_P36W_P90Y_R97A | 0.000 | 2.890 | 0.000 | 3.340 | 1.000 | 2.560 |
| 2243 | Y7L_I16L_P36W_P90Y_V93I | 0.000 | 7.750 | 0.000 | 6.640 | 0.000 | 6.610 |
| 2244 | Y7L_I16L_P36W_P90Y_V93I_R97A | 0.010 | 4.070 | 0.000 | 5.000 | 1.000 | 1.350 |
| 2245 | Y7L_I16L_P36W_P90Y_V93I_R97F | 0.010 | 2.580 | 0.000 | 2.850 | 1.000 | 2.290 |
| 2246 | Y7L_I16L_P36W_P90Y_V93I_R97H | 0.000 | 3.160 | 0.000 | 3.970 | 1.000 | 0.670 |
| 2247 | Y7L_I16L_P36W_P90Y_V93I_R97W | 1.000 | 1.230 | 0.000 | 2.910 | 1.000 | −1.240 |
| 2248 | Y7L_I16L_P36W_R97A | 0.000 | 4.560 | 0.000 | 4.070 | 0.000 | 3.690 |
| 2249 | Y7L_I16L_P36W_R97F | 0.000 | 4.220 | 0.000 | 3.040 | 0.600 | 2.420 |
| 2250 | Y7L_I16L_P36W_R97H | 0.000 | 5.870 | 0.000 | 5.150 | 0.000 | 4.910 |
| 2251 | Y7L_I16L_P36W_R97W | 1.000 | 2.010 | 0.170 | 2.670 | 0.010 | 3.480 |
| 2252 | Y7L_I16L_P36W_V93I | 0.000 | 10.560 | 0.000 | 8.040 | 0.000 | 8.180 |
| 2253 | Y7L_I16L_P36W_V93I_R97A | 0.000 | 3.710 | 0.460 | 3.480 | 0.800 | 2.260 |
| 2254 | Y7L_I16L_P36W_V93I_R97F | 0.000 | 5.580 | 0.000 | 5.400 | 0.000 | 4.020 |
| 2255 | Y7L_I16L_P36W_V93I_R97H | 0.000 | 5.350 | 0.000 | 5.490 | 0.020 | 3.760 |
| 2256 | Y7L_I16L_P36W_V93I_R97W | 0.000 | 3.400 | 0.000 | 2.640 | 0.000 | 2.450 |
| 2257 | Y7L_I16L_P36W_V93Q | 0.000 | 4.190 | 0.000 | 4.840 | 1.000 | 2.580 |
| 2258 | Y7L_I16L_P36W_V93Q_R97A | 1.000 | 0.230 | 0.000 | 3.000 | 0.370 | 2.750 |
| 2259 | Y7L_I16L_P36W_V93R | 0.000 | 3.480 | 0.010 | 2.530 | 1.000 | 1.680 |
| 2260 | Y7L_I16L_P90F | 0.000 | 8.840 | 0.000 | 7.090 | 0.000 | 7.990 |
| 2261 | Y7L_I16L_P90F_R97A | 0.000 | 4.810 | 0.010 | 4.440 | 0.310 | 2.140 |
| 2262 | Y7L_I16L_P90F_R97F | 0.000 | 5.030 | 0.430 | 3.120 | 0.000 | 2.760 |
| 2263 | Y7L_I16L_P90F_R97H | 0.000 | 4.220 | 0.000 | 4.550 | 0.000 | 3.390 |
| 2264 | Y7L_I16L_P90F_R97W | 1.000 | 2.160 | 0.000 | 2.890 | 1.000 | 0.720 |
| 2265 | Y7L_I16L_P90F_V93I | 0.000 | 9.260 | 0.000 | 8.800 | 0.000 | 9.840 |
| 2266 | Y7L_I16L_P90F_V93I_R97A | 0.000 | 5.480 | 0.000 | 5.420 | 0.000 | 4.610 |
| 2267 | Y7L_I16L_P90F_V93I_R97F | 0.000 | 4.620 | 0.000 | 4.870 | 0.010 | 3.010 |
| 2268 | Y7L_I16L_P90F_V93I_R97H | 0.000 | 6.360 | 0.000 | 5.670 | 0.000 | 4.930 |
| 2269 | Y7L_I16L_P90F_V93I_R97W | 0.000 | 4.340 | 0.640 | 2.670 | 1.000 | 2.500 |
| 2270 | Y7L_I16L_P90F_V93Q | 0.010 | 4.600 | 0.160 | 3.080 | 1.000 | 1.150 |
| 2271 | Y7L_I16L_P90F_V93R_R97F | 0.000 | 4.050 | 0.000 | 3.000 | 0.000 | 3.320 |
| 2272 | Y7L_I16L_P90I | 0.000 | 6.640 | 1.000 | 2.620 | 0.000 | 3.040 |
| 2273 | Y7L_I16L_P90I_R97A | 0.000 | 4.990 | 0.030 | 3.960 | 0.180 | 2.720 |
| 2274 | Y7L_I16L_P90I_R97F | 1.000 | 2.170 | 0.000 | 2.620 | 1.000 | 1.120 |
| 2275 | Y7L_I16L_P90I_R97H | 0.000 | 4.190 | 0.000 | 3.830 | 0.000 | 4.810 |
| 2276 | Y7L_I16L_P90I_V93I | 0.000 | 8.670 | 0.000 | 4.100 | 0.000 | 4.000 |
| 2277 | Y7L_I16L_P90I_V93I_R97A | 0.000 | 4.240 | 0.000 | 4.520 | 1.000 | 3.330 |
| 2278 | Y7L_I16L_P90I_V93I_R97F | 0.010 | 5.080 | 0.000 | 4.380 | 0.200 | 3.330 |
| 2279 | Y7L_I16L_P90I_V93I_R97H | 0.000 | 5.020 | 0.000 | 4.500 | 0.000 | 4.900 |
| 2280 | Y7L_I16L_P90I_V93I_R97W | 0.140 | 3.450 | 0.000 | 2.640 | 1.000 | 1.910 |
| 2281 | Y7L_I16L_P90I_V93Q | 0.030 | 4.620 | 0.000 | 3.200 | 1.000 | 0.550 |
| 2282 | Y7L_I16L_P90S_V93I | 0.000 | 3.430 | 0.010 | 1.340 | 0.000 | 2.580 |
| 2283 | Y7L_I16L_P90S_V93I_R97A | 0.000 | 4.240 | 0.000 | 3.220 | 0.280 | 3.380 |
| 2284 | Y7L_I16L_P90S_V93I_R97H | 0.000 | 3.980 | 0.000 | 3.350 | 1.000 | 2.430 |
| 2285 | Y7L_I16L_P90W | 0.000 | 7.740 | 0.000 | 7.360 | 0.000 | 7.420 |
| 2286 | Y7L_I16L_P90W_R97A | 0.010 | 4.800 | 0.000 | 6.190 | 0.000 | 3.300 |
| 2287 | Y7L_I16L_P90W_R97F | 0.000 | 4.810 | 0.000 | 4.430 | 0.000 | 3.760 |
| 2288 | Y7L_I16L_P90W_R97H | 0.000 | 5.430 | 0.000 | 5.830 | 0.030 | 3.380 |
| 2289 | Y7L_I16L_P90W_R97W | 0.020 | 3.100 | 0.300 | 2.360 | 0.390 | 1.750 |
| 2290 | Y7L_I16L_P90W_V93I | 0.000 | 9.170 | 0.000 | 8.670 | 0.000 | 8.860 |
| 2291 | Y7L_I16L_P90W_V93I_R97A | 0.000 | 6.070 | 0.000 | 6.810 | 0.000 | 4.880 |
| 2292 | Y7L_I16L_P90W_V93I_R97F | 0.000 | 6.310 | 0.000 | 6.330 | 0.000 | 3.310 |
| 2293 | Y7L_I16L_P90W_V93I_R97H | 0.000 | 5.800 | 0.000 | 5.160 | 0.610 | 3.140 |
| 2294 | Y7L_I16L_P90W_V93I_R97W | 0.000 | 4.950 | 0.000 | 4.630 | 0.030 | 3.230 |
| 2295 | Y7L_I16L_P90W_V93K_R97A | 0.000 | 2.830 | 0.000 | 3.000 | 0.000 | 3.330 |
| 2296 | Y7L_I16L_P90W_V93Q | 0.000 | 4.590 | 0.010 | 2.580 | 1.000 | 2.760 |
| 2297 | Y7L_I16L_P90W_V93Q_R97A | 0.000 | 4.000 | 0.480 | 2.780 | 1.000 | 1.960 |
| 2298 | Y7L_I16L_P90W_V93R_R97A | 0.000 | 3.740 | 0.140 | 4.450 | 0.030 | 4.000 |
| 2299 | Y7L_I16L_P90W_V93R_R97H | 0.000 | 3.180 | 0.000 | 2.630 | 0.000 | 2.520 |
| 2300 | Y7L_I16L_P90Y | 0.000 | 8.480 | 0.000 | 7.960 | 0.000 | 6.800 |
| 2301 | Y7L_I16L_P90Y_R97A | 0.000 | 6.670 | 0.000 | 5.650 | 0.000 | 4.550 |
| 2302 | Y7L_I16L_P90Y_R97F | 0.000 | 4.150 | 0.000 | 3.870 | 0.100 | 2.700 |
| 2303 | Y7L_I16L_P90Y_R97H | 0.000 | 5.110 | 0.000 | 4.610 | 1.000 | 1.730 |
| 2304 | Y7L_I16L_P90Y_V93I | 0.000 | 8.330 | 0.000 | 7.930 | 0.000 | 7.640 |
| 2305 | Y7L_I16L_P90Y_V93I_R97A | 0.000 | 6.220 | 0.000 | 5.750 | 0.010 | 2.150 |
| 2306 | Y7L_I16L_P90Y_V93I_R97F | 0.000 | 5.120 | 0.140 | 4.100 | 1.000 | 1.330 |
| 2307 | Y7L_I16L_P90Y_V93I_R97H | 0.000 | 6.080 | 0.000 | 6.090 | 0.000 | 4.830 |
| 2308 | Y7L_I16L_P90Y_V93I_R97W | 0.000 | 3.950 | 0.010 | 4.480 | 0.610 | 2.690 |
| 2309 | Y7L_I16L_P90Y_V93K | 0.000 | 2.780 | 0.040 | 1.500 | 0.010 | 2.230 |
| 2310 | Y7L_I16L_P90Y_V93Q | 0.000 | 6.260 | 0.000 | 5.710 | 0.000 | 5.120 |
| 2311 | Y7L_I16L_P90Y_V93Q_R97F | 0.000 | 3.250 | 0.000 | 2.710 | 0.030 | 2.890 |
| 2312 | Y7L_I16L_R97A | 0.000 | 6.430 | 0.000 | 4.860 | 0.000 | 5.370 |
| 2313 | Y7L_I16L_R97F | 0.000 | 4.790 | 0.000 | 5.300 | 0.000 | 4.780 |
| 2314 | Y7L_I16L_R97H | 0.000 | 4.990 | 0.000 | 5.870 | 0.000 | 4.880 |
| 2315 | Y7L_I16L_R97W | 0.000 | 6.300 | 0.000 | 6.230 | 0.080 | 3.530 |
| 2316 | Y7L_I16L_V93I | 0.000 | 7.310 | 0.000 | 2.660 | 0.000 | 2.910 |
| 2317 | Y7L_I16L_V93I_R97A | 0.000 | 6.440 | 0.000 | 4.520 | 0.000 | 5.360 |
| 2318 | Y7L_I16L_V93I_R97F | 0.000 | 5.820 | 0.000 | 5.550 | 0.000 | 5.380 |
| 2319 | Y7L_I16L_V93I_R97H | 0.000 | 5.990 | 0.000 | 5.480 | 0.000 | 5.910 |
| 2320 | Y7L_I16L_V93I_R97W | 0.000 | 4.720 | 0.000 | 5.670 | 0.000 | 5.300 |
| 2321 | Y7L_I16L_V93K | 0.000 | 5.220 | 0.000 | 4.220 | 0.010 | 1.910 |
| 2322 | Y7L_I16L_V93Q | 0.000 | 7.440 | 0.000 | 6.700 | 0.000 | 5.060 |
| 2323 | Y7L_I16L_V93Q_R97A | 0.020 | 4.450 | 0.000 | 4.850 | 1.000 | 1.270 |
| 2324 | Y7L_I16L_V93Q_R97F | 0.000 | 4.050 | 0.000 | 3.830 | 1.000 | 2.560 |
| 2325 | Y7L_I16L_V93Q_R97H | 0.020 | 2.470 | 0.100 | 1.050 | 1.000 | 0.840 |
| 2326 | Y7L_I16L_V93Q_R97W | 0.000 | 4.660 | 0.000 | 4.170 | 0.010 | 3.060 |
| 2327 | Y7L_I16L_V93R | 0.000 | 4.180 | 0.000 | 4.940 | 0.000 | 3.820 |
| 2328 | Y7L_I16L_V93R_R97A | 0.000 | 5.230 | 0.000 | 4.460 | 1.000 | 1.550 |
| 2329 | Y7L_I16L_V93R_R97F | 0.000 | 4.610 | 0.000 | 3.440 | 1.000 | 1.720 |
| 2330 | Y7L_I16L_V93R_R97H | 0.000 | 4.220 | 0.000 | 4.680 | 0.000 | 4.120 |
| 2331 | Y7L_I16L_V93R_R97W | 0.040 | 3.190 | 1.000 | 1.560 | 1.000 | 1.100 |
| 2332 | Y7L_I16M | 0.000 | 4.960 | 0.000 | 2.900 | 0.000 | 2.670 |
| 2333 | Y7L_I16M_P36A | 0.000 | 5.470 | 1.000 | 0.520 | 0.560 | 2.170 |
| 2334 | Y7L_I16M_P36A_P90F | 0.070 | 3.440 | 0.080 | 2.820 | 0.020 | 4.030 |
| 2335 | Y7L_I16M_P36A_P90F_V93I | 0.310 | 2.700 | 0.240 | 2.440 | 0.000 | 3.580 |
| 2336 | Y7L_I16M_P36A_P90I_V93I_R97W | 0.000 | 3.010 | 1.000 | 1.540 | 0.010 | 2.350 |
| 2337 | Y7L_I16M_P36A_P90W_V93I | 0.000 | 4.450 | 0.000 | 2.920 | 0.000 | 3.430 |
| 2338 | Y7L_I16M_P36A_P90Y | 0.000 | 3.640 | 1.000 | 1.670 | 1.000 | −0.300 |
| 2339 | Y7L_I16M_P36A_P90Y_V93I | 0.030 | 3.110 | 0.000 | 3.270 | 0.000 | 3.860 |
| 2340 | Y7L_I16M_P36A_P90Y_V93I_R97A | 1.000 | 1.780 | 0.000 | 3.420 | 1.000 | −1.010 |
| 2341 | Y7L_I16M_P36A_R97F | 0.000 | 4.490 | 1.000 | 1.660 | 1.000 | 0.770 |
| 2342 | Y7L_I16M_P36A_V93I | 0.000 | 5.850 | 1.000 | 0.660 | 1.000 | 0.300 |
| 2343 | Y7L_I16M_P36A_V93I_R97A | 0.000 | 4.630 | 0.000 | 3.220 | 0.000 | 3.810 |
| 2344 | Y7L_I16M_P36A_V93I_R97H | 0.000 | 3.690 | 0.000 | 3.420 | 0.000 | 3.380 |
| 2345 | Y7L_I16M_P36A_V93I_R97W | 1.000 | 2.200 | 0.000 | 2.590 | 1.000 | 1.860 |
| 2346 | Y7L_I16M_P36A_V93Q | 0.390 | 3.570 | 0.010 | 2.970 | 1.000 | 2.060 |
| 2347 | Y7L_I16M_P36A_V93R_R97A | 0.000 | 4.890 | 0.000 | 4.390 | 1.000 | −0.370 |
| 2348 | Y7L_I16M_P36H | 0.060 | 4.380 | 0.000 | 2.890 | 0.000 | 3.260 |
| 2349 | Y7L_I16M_P36H_R97A | 0.000 | 3.850 | 1.000 | −0.640 | 1.000 | −2.920 |
| 2350 | Y7L_I16M_P36H_V93I | 0.000 | 5.820 | 0.000 | 5.550 | 0.110 | 2.900 |
| 2351 | Y7L_I16M_P36H_V93R | 0.000 | 4.160 | 0.000 | 2.740 | 0.000 | 2.710 |
| 2352 | Y7L_I16M_P36S | 0.030 | 3.440 | 1.000 | −1.520 | 1.000 | 0.260 |
| 2353 | Y7L_I16M_P36S_P90W | 0.000 | 4.460 | 0.000 | 2.870 | 0.000 | 3.880 |
| 2354 | Y7L_I16M_P36S_R97A | 1.000 | 2.030 | 0.000 | 3.500 | 1.000 | 0.340 |
| 2355 | Y7L_I16M_P36S_V93I | 0.030 | 4.690 | 1.000 | −0.770 | 1.000 | 0.370 |
| 2356 | Y7L_I16M_P36S_V93I_R97H | 0.000 | 3.920 | 1.000 | 2.040 | 1.000 | −1.380 |
| 2357 | Y7L_I16M_P36W | 0.000 | 7.060 | 0.000 | 7.000 | 0.000 | 7.260 |
| 2358 | Y7L_I16M_P36W_P90F | 0.000 | 5.230 | 0.000 | 4.840 | 0.020 | 2.680 |
| 2359 | Y7L_I16M_P36W_P90F_R97A | 0.000 | 3.670 | 0.000 | 3.810 | 0.000 | 3.730 |
| 2360 | Y7L_I16M_P36W_P90F_R97H | 0.040 | 2.660 | 0.310 | 1.740 | 1.000 | 1.200 |
| 2361 | Y7L_I16M_P36W_P90F_V93I | 0.000 | 5.460 | 0.000 | 6.750 | 0.000 | 5.570 |
| 2362 | Y7L_I16M_P36W_P90F_V93I_R97A | 0.000 | 4.000 | 1.000 | 2.270 | 1.000 | 2.080 |
| 2363 | Y7L_I16M_P36W_P90F_V93I_R97F | 0.000 | 3.980 | 0.370 | 2.810 | 0.460 | 3.150 |
| 2364 | Y7L_I16M_P36W_P90F_V93I_R97H | 0.000 | 2.990 | 0.000 | 2.700 | 0.000 | 2.220 |
| 2365 | Y7L_I16M_P36W_P90I_V93I | 0.000 | 6.240 | 0.010 | 4.070 | 0.000 | 4.630 |
| 2366 | Y7L_I16M_P36W_P90I_V93I_R97H | 0.000 | 3.270 | 0.240 | 1.890 | 1.000 | −0.610 |
| 2367 | Y7L_I16M_P36W_P90W | 0.000 | 5.420 | 0.000 | 5.640 | 0.000 | 4.470 |
| 2368 | Y7L_I16M_P36W_P90W_R97A | 0.000 | 3.160 | 1.000 | 0.510 | 1.000 | 1.470 |
| 2369 | Y7L_I16M_P36W_P90W_V93I | 0.000 | 5.780 | 0.000 | 5.990 | 0.000 | 4.420 |
| 2370 | Y7L_I16M_P36W_P90W_V93I_R97A | 0.000 | 4.170 | 0.000 | 3.950 | 1.000 | 1.470 |
| 2371 | Y7L_I16M_P36W_P90W_V93I_R97F | 0.000 | 2.320 | 0.020 | 3.040 | 1.000 | 1.200 |
| 2372 | Y7L_I16M_P36W_P90W_V93I_R97H | 0.000 | 3.490 | 0.000 | 3.360 | 1.000 | 1.700 |
| 2373 | Y7L_I16M_P36W_P90Y | 0.000 | 5.040 | 0.000 | 6.780 | 0.000 | 3.880 |
| 2374 | Y7L_I16M_P36W_P90Y_R97A | 0.000 | 3.990 | 0.000 | 3.560 | 0.050 | 3.160 |
| 2375 | Y7L_I16M_P36W_P90Y_R97F | 0.000 | 3.440 | 0.750 | 1.950 | 1.000 | 1.500 |
| 2376 | Y7L_I16M_P36W_P90Y_V93I | 0.000 | 6.560 | 0.000 | 5.990 | 0.000 | 4.500 |
| 2377 | Y7L_I16M_P36W_P90Y_V93I_R97A | 0.000 | 4.760 | 0.000 | 3.570 | 0.090 | 2.900 |
| 2378 | Y7L_I16M_P36W_P90Y_V93I_R97H | 0.000 | 4.950 | 0.050 | 3.250 | 0.020 | 2.830 |
| 2379 | Y7L_I16M_P36W_R97A | 0.000 | 4.670 | 0.020 | 4.190 | 0.000 | 4.900 |
| 2380 | Y7L_I16M_P36W_R97F | 0.000 | 3.570 | 0.010 | 2.910 | 1.000 | 1.170 |
| 2381 | Y7L_I16M_P36W_R97H | 0.000 | 6.240 | 0.000 | 5.810 | 0.000 | 5.700 |
| 2382 | Y7L_I16M_P36W_R97W | 0.000 | 4.530 | 0.010 | 4.390 | 1.000 | 1.560 |
| 2383 | Y7L_I16M_P36W_V93I | 0.000 | 8.750 | 0.000 | 6.200 | 0.000 | 7.400 |
| 2384 | Y7L_I16M_P36W_V93I_R97A | 0.010 | 3.680 | 0.000 | 4.790 | 1.000 | 1.750 |
| 2385 | Y7L_I16M_P36W_V93I_R97F | 0.000 | 4.990 | 0.000 | 5.590 | 0.180 | 3.300 |
| 2386 | Y7L_I16M_P36W_V93I_R97H | 0.000 | 4.760 | 0.000 | 4.730 | 0.000 | 4.350 |
| 2387 | Y7L_I16M_P36W_V93I_R97W | 0.000 | 5.040 | 0.000 | 4.520 | 0.000 | 3.900 |
| 2388 | Y7L_I16M_P36W_V93K | 0.000 | 3.220 | 0.000 | 2.920 | 1.000 | −0.830 |
| 2389 | Y7L_I16M_P36W_V93Q | 0.000 | 3.530 | 0.700 | 2.700 | 1.000 | 2.310 |
| 2390 | Y7L_I16M_P36W_V93Q_R97A | 0.000 | 3.320 | 0.000 | 2.940 | 0.140 | 2.210 |
| 2391 | Y7L_I16M_P36W_V93Q_R97F | 0.000 | 2.820 | 0.010 | 2.390 | 1.000 | −1.360 |
| 2392 | Y7L_I16M_P36W_V93Q_R97W | 0.000 | 2.660 | 0.770 | 1.190 | 1.000 | −0.020 |
| 2393 | Y7L_I16M_P36W_V93R | 0.000 | 3.740 | 0.000 | 4.980 | 1.000 | 1.150 |
| 2394 | Y7L_I16M_P36W_V93R_R97A | 0.010 | 4.650 | 0.000 | 5.270 | 0.050 | 3.860 |
| 2395 | Y7L_I16M_P36W_V93R_R97A_R119Q | 0.000 | 1.460 | 0.000 | 2.070 | 1.000 | 0.610 |
| 2396 | Y7L_I16M_P36W_V93R_R97F | 0.000 | 2.340 | 1.000 | −0.160 | 1.000 | 0.070 |
| 2397 | Y7L_I16M_P90F | 0.000 | 7.770 | 0.000 | 6.740 | 0.000 | 7.190 |
| 2398 | Y7L_I16M_P90F_R97A | 0.000 | 4.310 | 0.000 | 4.990 | 0.000 | 3.710 |
| 2399 | Y7L_I16M_P90F_R97F | 0.010 | 4.200 | 0.040 | 3.570 | 1.000 | 2.580 |
| 2400 | Y7L_I16M_P90F_R97H | 0.000 | 4.480 | 0.000 | 4.110 | 0.400 | 2.650 |
| 2401 | Y7L_I16M_P90F_R97W | 0.070 | 2.560 | 0.000 | 3.530 | 1.000 | 2.210 |
| 2402 | Y7L_I16M_P90F_V93I | 0.000 | 8.030 | 0.000 | 7.660 | 0.000 | 8.460 |
| 2403 | Y7L_I16M_P90F_V93I_R97A | 0.020 | 4.600 | 0.000 | 5.890 | 0.010 | 3.280 |
| 2404 | Y7L_I16M_P90F_V93I_R97F | 0.000 | 3.900 | 0.050 | 3.130 | 0.530 | 1.910 |
| 2405 | Y7L_I16M_P90F_V93I_R97H | 0.000 | 5.780 | 0.000 | 4.390 | 0.000 | 3.930 |
| 2406 | Y7L_I16M_P90F_V93I_R97W | 0.000 | 3.640 | 0.000 | 3.410 | 1.000 | 1.500 |
| 2407 | Y7L_I16M_P90F_V93Q | 0.000 | 3.580 | 0.130 | 2.110 | 0.020 | 3.370 |
| 2408 | Y7L_I16M_P90F_V93Q_R97A | 1.000 | 2.730 | 0.000 | 3.170 | 1.000 | 0.270 |
| 2409 | Y7L_I16M_P90F_V93R_R97A | 0.000 | 3.080 | 1.000 | 0.340 | 1.000 | 0.900 |
| 2410 | Y7L_I16M_P90I | 0.000 | 8.410 | 0.000 | 6.260 | 0.000 | 7.270 |
| 2411 | Y7L_I16M_P90I_R97A | 0.010 | 4.450 | 1.000 | 1.450 | 1.000 | 0.790 |
| 2412 | Y7L_I16M_P90I_R97F | 0.000 | 3.660 | 0.000 | 3.230 | 0.000 | 3.080 |
| 2413 | Y7L_I16M_P90I_R97H | 0.000 | 6.230 | 0.000 | 5.400 | 0.000 | 5.070 |
| 2414 | Y7L_I16M_P90I_R97W | 0.300 | 2.560 | 0.030 | 2.250 | 1.000 | 0.960 |
| 2415 | Y7L_I16M_P90I_V93I | 0.000 | 9.190 | 0.000 | 5.790 | 0.000 | 6.210 |
| 2416 | Y7L_I16M_P90I_V93I_R97A | 0.000 | 5.000 | 0.000 | 4.850 | 0.000 | 4.860 |
| 2417 | Y7L_I16M_P90I_V93I_R97F | 0.000 | 2.580 | 0.000 | 3.250 | 0.000 | 3.580 |
| 2418 | Y7L_I16M_P90I_V93I_R97H | 0.000 | 4.570 | 0.000 | 3.970 | 0.000 | 4.630 |
| 2419 | Y7L_I16M_P90I_V93I_R97W | 0.040 | 3.400 | 0.000 | 3.140 | 0.000 | 3.640 |
| 2420 | Y7L_I16M_P90I_V93Q | 0.000 | 6.390 | 0.000 | 5.170 | 0.000 | 3.430 |
| 2421 | Y7L_I16M_P90I_V93R | 0.000 | 6.710 | 0.010 | 5.570 | 0.770 | 2.980 |
| 2422 | Y7L_I16M_P90I_V93R_R97A | 1.000 | 0.480 | 0.000 | 4.820 | 1.000 | 0.280 |
| 2423 | Y7L_I16M_P90I_V93R_R97F | 0.010 | 2.450 | 0.000 | 1.870 | 1.000 | −0.150 |
| 2424 | Y7L_I16M_P90S_R97F | 0.050 | 2.670 | 1.000 | −2.260 | 1.000 | 0.450 |
| 2425 | Y7L_I16M_P90S_V93I | 0.010 | 4.320 | 1.000 | −0.810 | 1.000 | −1.080 |
| 2426 | Y7L_I16M_P90S_V93I_R97A | 0.030 | 3.240 | 1.000 | 0.530 | 1.000 | 2.240 |
| 2427 | Y7L_I16M_P90S_V93I_R97H | 0.000 | 4.040 | 1.000 | 2.220 | 1.000 | 1.190 |
| 2428 | Y7L_I16M_P90W | 0.000 | 5.740 | 0.000 | 4.740 | 0.000 | 6.060 |
| 2429 | Y7L_I16M_P90W_R97A | 0.000 | 4.160 | 0.000 | 4.840 | 0.380 | 2.900 |
| 2430 | Y7L_I16M_P90W_R97F | 0.000 | 3.420 | 0.000 | 3.900 | 1.000 | 1.780 |
| 2431 | Y7L_I16M_P90W_R97H | 0.000 | 3.360 | 0.020 | 4.130 | 1.000 | 1.710 |
| 2432 | Y7L_I16M_P90W_V93I | 0.000 | 9.270 | 0.000 | 8.440 | 0.000 | 8.690 |
| 2433 | Y7L_I16M_P90W_V93I_R97A | 0.000 | 7.050 | 0.000 | 6.240 | 0.000 | 4.710 |
| 2434 | Y7L_I16M_P90W_V93I_R97F | 0.000 | 4.520 | 0.030 | 4.020 | 0.000 | 3.210 |
| 2435 | Y7L_I16M_P90W_V93I_R97H | 0.000 | 5.160 | 0.000 | 5.190 | 0.010 | 3.210 |
| 2436 | Y7L_I16M_P90W_V93I_R97W | 0.000 | 3.740 | 0.000 | 2.540 | 0.000 | 2.140 |
| 2437 | Y7L_I16M_P90W_V93K | 0.000 | 3.410 | 0.000 | 2.310 | 0.000 | 1.820 |
| 2438 | Y7L_I16M_P90W_V93Q_R97A | 0.000 | 2.300 | 1.000 | 1.110 | 1.000 | 0.290 |
| 2439 | Y7L_I16M_P90W_V93R_R97H | 0.160 | 3.040 | 0.000 | 3.350 | 0.000 | 4.700 |
| 2440 | Y7L_I16M_P90Y | 0.000 | 7.670 | 0.000 | 5.000 | 0.000 | 4.390 |
| 2441 | Y7L_I16M_P90Y_R97A | 0.540 | 2.840 | 0.030 | 3.620 | 0.000 | 3.900 |
| 2442 | Y7L_I16M_P90Y_R97F | 0.050 | 3.190 | 1.000 | 1.910 | 1.000 | 2.740 |
| 2443 | Y7L_I16M_P90Y_R97W | 0.000 | 3.910 | 1.000 | 1.830 | 1.000 | 1.480 |
| 2444 | Y7L_I16M_P90Y_V93I | 0.000 | 8.020 | 0.000 | 6.460 | 0.000 | 7.520 |
| 2445 | Y7L_I16M_P90Y_V93I_R97A | 0.140 | 4.030 | 0.000 | 6.130 | 0.020 | 3.310 |
| 2446 | Y7L_I16M_P90Y_V93I_R97F | 0.000 | 5.720 | 0.000 | 5.250 | 1.000 | 2.580 |
| 2447 | Y7L_I16M_P90Y_V93I_R97H | 0.000 | 5.080 | 0.000 | 5.610 | 0.600 | 3.550 |
| 2448 | Y7L_I16M_P90Y_V93I_R97W | 0.040 | 3.840 | 0.020 | 3.690 | 1.000 | 1.030 |
| 2449 | Y7L_I16M_P90Y_V93Q | 0.000 | 5.300 | 1.000 | 2.510 | 1.000 | 1.450 |
| 2450 | Y7L_I16M_R97A | 0.000 | 4.710 | 0.000 | 4.930 | 0.000 | 4.910 |
| 2451 | Y7L_I16M_R97F | 0.000 | 4.780 | 0.520 | 3.530 | 1.000 | 2.100 |
| 2452 | Y7L_I16M_R97H | 0.000 | 5.250 | 0.000 | 5.500 | 0.000 | 4.750 |
| 2453 | Y7L_I16M_R97W | 0.000 | 4.810 | 0.000 | 4.570 | 0.000 | 5.310 |
| 2454 | Y7L_I16M_V93I | 0.000 | 5.970 | 0.030 | 1.570 | 0.020 | 1.780 |
| 2455 | Y7L_I16M_V93I_R97A | 0.000 | 6.340 | 0.000 | 5.620 | 0.000 | 4.150 |
| 2456 | Y7L_I16M_V93I_R97F | 0.000 | 5.020 | 0.000 | 5.470 | 0.000 | 3.800 |
| 2457 | Y7L_I16M_V93I_R97H | 0.000 | 5.450 | 0.000 | 5.560 | 0.000 | 4.970 |
| 2458 | Y7L_I16M_V93I_R97W | 0.000 | 3.820 | 0.000 | 3.430 | 0.000 | 3.470 |
| 2459 | Y7L_I16M_V93K | 0.000 | 3.790 | 0.010 | 3.110 | 1.000 | 1.060 |
| 2460 | Y7L_I16M_V93K_R97A | 0.000 | 2.530 | 0.000 | 2.390 | 0.000 | 2.030 |
| 2461 | Y7L_I16M_V93K_R97W | 0.010 | 2.750 | 1.000 | 1.030 | 1.000 | −0.140 |
| 2462 | Y7L_I16M_V93Q | 0.000 | 6.650 | 0.000 | 6.290 | 0.000 | 5.360 |
| 2463 | Y7L_I16M_V93Q_R97A | 0.000 | 4.120 | 0.000 | 4.030 | 1.000 | 1.130 |
| 2464 | Y7L_I16M_V93Q_R97F | 0.000 | 3.820 | 0.000 | 3.550 | 1.000 | 1.480 |
| 2465 | Y7L_I16M_V93Q_R97H | 0.000 | 2.660 | 1.000 | 1.460 | 1.000 | −0.290 |
| 2466 | Y7L_I16M_V93R | 0.000 | 6.720 | 0.000 | 4.980 | 0.000 | 3.450 |
| 2467 | Y7L_I16M_V93R_R97A | 0.000 | 6.260 | 0.000 | 7.080 | 0.430 | 3.170 |
| 2468 | Y7L_I16W | 0.000 | 7.560 | 0.000 | 5.570 | 0.000 | 5.070 |
| 2469 | Y7L_I16W_P36A | 0.000 | 5.580 | 0.150 | 2.680 | 0.000 | 6.500 |
| 2470 | Y7L_I16W_P36A_R97A | 0.000 | 4.120 | 1.000 | 1.400 | 1.000 | 0.480 |
| 2471 | Y7L_I16W_P36A_V93I | 0.000 | 8.120 | 0.000 | 5.920 | 0.000 | 6.670 |
| 2472 | Y7L_I16W_P36A_V93I_R97A | 0.170 | 3.160 | 0.010 | 4.520 | 0.020 | 3.240 |
| 2473 | Y7L_I16W_P36A_V93I_R97F | 0.000 | 3.590 | 0.000 | 3.050 | 1.000 | 2.090 |
| 2474 | Y7L_I16W_P36A_V93I_R97H | 1.000 | 1.280 | 0.000 | 4.300 | 1.000 | 2.080 |
| 2475 | Y7L_I16W_P36A_V93I_R97W | 0.000 | 2.360 | 0.000 | 1.920 | 0.000 | 2.120 |
| 2476 | Y7L_I16W_P36A_V93R | 0.010 | 4.430 | 0.000 | 3.240 | 0.000 | 3.670 |
| 2477 | Y7L_I16W_P36H | 0.010 | 3.760 | 1.000 | 1.380 | 1.000 | 0.980 |
| 2478 | Y7L_I16W_P36H_V93I | 0.000 | 5.600 | 0.000 | 4.400 | 0.540 | 3.380 |
| 2479 | Y7L_I16W_P36S_V93I | 0.000 | 5.150 | 0.670 | 4.190 | 0.000 | 5.110 |
| 2480 | Y7L_I16W_P36W_P90Y_V93I | 0.000 | 2.190 | 0.020 | 1.580 | 0.000 | 1.850 |
| 2481 | Y7L_I16W_P36W_R97A | 0.000 | 2.320 | 0.000 | 3.170 | 1.000 | 1.450 |
| 2482 | Y7L_I16W_P36W_V93I | 0.650 | 3.030 | 1.000 | 2.190 | 0.040 | 3.190 |
| 2483 | Y7L_I16W_P90F | 0.000 | 3.010 | 0.380 | 2.540 | 0.000 | 4.170 |
| 2484 | Y7L_I16W_P90F_R97A | 0.000 | 3.650 | 0.000 | 2.910 | 0.000 | 3.440 |
| 2485 | Y7L_I16W_P90F_V93I | 0.000 | 6.180 | 0.000 | 4.970 | 0.000 | 3.860 |
| 2486 | Y7L_I16W_P90F_V93I_R97A | 0.000 | 6.120 | 0.000 | 5.700 | 0.000 | 4.950 |
| 2487 | Y7L_I16W_P90F_V93I_R97H | 0.000 | 4.450 | 0.000 | 3.090 | 1.000 | 1.520 |
| 2488 | Y7L_I16W_P90I | 0.000 | 5.330 | 1.000 | 2.970 | 1.000 | 2.440 |
| 2489 | Y7L_I16W_P90I_V93I | 0.000 | 6.660 | 0.000 | 5.840 | 0.000 | 3.940 |
| 2490 | Y7L_I16W_P90W | 0.000 | 2.910 | 0.480 | 1.590 | 0.000 | 1.770 |
| 2491 | Y7L_I16W_P90W_V93I | 0.000 | 3.690 | 0.540 | 2.240 | 1.000 | 1.460 |
| 2492 | Y7L_I16W_P90W_V93I_R97A | 0.000 | 5.900 | 0.000 | 5.500 | 0.000 | 4.920 |
| 2493 | Y7L_I16W_P90W_V93I_R97W | 0.000 | 4.150 | 0.000 | 2.970 | 0.000 | 2.890 |
| 2494 | Y7L_I16W_P90Y_V93I | 0.000 | 5.900 | 0.000 | 4.350 | 0.000 | 5.190 |
| 2495 | Y7L_I16W_R97A | 0.000 | 6.310 | 0.000 | 5.190 | 0.000 | 5.750 |
| 2496 | Y7L_I16W_R97F | 0.000 | 5.860 | 0.000 | 5.580 | 0.140 | 3.610 |
| 2497 | Y7L_I16W_R97H | 0.000 | 5.510 | 0.000 | 5.870 | 0.000 | 4.670 |
| 2498 | Y7L_I16W_R97W | 0.030 | 2.760 | 0.000 | 3.260 | 0.000 | 2.930 |
| 2499 | Y7L_I16W_V93I | 0.000 | 8.180 | 0.000 | 3.990 | 0.000 | 4.190 |
| 2500 | Y7L_I16W_V93I_R97F | 0.000 | 5.380 | 0.000 | 5.000 | 0.000 | 4.980 |
| 2501 | Y7L_I16W_V93I_R97H | 0.000 | 5.080 | 0.000 | 4.910 | 0.010 | 4.300 |
| 2502 | Y7L_I16W_V93I_R97W | 0.000 | 5.720 | 0.000 | 5.000 | 0.000 | 4.530 |
| 2503 | Y7L_I16W_V93Q | 0.000 | 3.480 | 0.010 | 3.170 | 0.180 | 2.740 |
| 2504 | Y7L_I16W_V93R | 0.000 | 2.350 | 0.090 | 1.590 | 1.000 | 2.080 |
| 2505 | Y7L_P36A | 0.000 | 6.850 | 1.000 | 1.420 | 1.000 | 0.900 |
| 2506 | Y7L_P36A_P90F | 0.020 | 4.580 | 0.000 | 3.990 | 0.000 | 4.630 |
| 2507 | Y7L_P36A_P90F_R97A | 0.000 | 3.710 | 0.000 | 3.580 | 1.000 | 0.240 |
| 2508 | Y7L_P36A_P90F_R97F | 0.000 | 2.980 | 0.000 | 2.770 | 0.000 | 2.810 |
| 2509 | Y7L_P36A_P90F_V93I | 0.000 | 6.200 | 0.000 | 6.600 | 0.000 | 6.130 |
| 2510 | Y7L_P36A_P90F_V93I_R97F | 0.000 | 3.400 | 1.000 | 1.380 | 1.000 | −0.510 |
| 2511 | Y7L_P36A_P90F_V93I_R97H | 0.000 | 2.940 | 0.110 | 2.670 | 0.000 | 3.240 |
| 2512 | Y7L_P36A_P90F_V93I_R97W | 1.000 | 2.030 | 0.010 | 2.360 | 1.000 | −1.270 |
| 2513 | Y7L_P36A_P90F_V93R | 0.000 | 2.630 | 0.020 | 1.880 | 0.020 | 1.720 |
| 2514 | Y7L_P36A_P90I | 0.000 | 5.790 | 0.000 | 3.220 | 0.010 | 3.410 |
| 2515 | Y7L_P36A_P90I_R97F | 0.000 | 4.480 | 0.000 | 3.200 | 0.000 | 3.410 |
| 2516 | Y7L_P36A_P90I_V93I | 0.000 | 6.160 | 0.000 | 3.190 | 0.000 | 2.910 |
| 2517 | Y7L_P36A_P90W | 0.000 | 5.630 | 0.000 | 6.410 | 0.000 | 5.480 |
| 2518 | Y7L_P36A_P90W_R97A | 0.150 | 3.080 | 0.000 | 3.950 | 1.000 | −0.790 |
| 2519 | Y7L_P36A_P90W_V93I | 0.000 | 4.010 | 0.000 | 3.050 | 0.000 | 2.820 |
| 2520 | Y7L_P36A_P90W_V93I_R97A | 0.000 | 3.870 | 0.640 | 3.250 | 1.000 | 2.280 |
| 2521 | Y7L_P36A_P90W_V93I_R97H | 0.000 | 4.500 | 0.000 | 4.870 | 1.000 | 1.930 |
| 2522 | Y7L_P36A_P90W_V93I_R97W | 0.000 | 4.140 | 0.040 | 4.800 | 0.000 | 3.130 |
| 2523 | Y7L_P36A_P90Y_V93I | 0.000 | 7.540 | 0.000 | 5.590 | 0.000 | 5.710 |
| 2524 | Y7L_P36A_P90Y_V93I_R97A | 1.000 | 2.520 | 0.000 | 3.470 | 1.000 | −1.450 |
| 2525 | Y7L_P36A_P90Y_V93I_R97F | 0.000 | 4.340 | 0.000 | 3.830 | 0.000 | 3.420 |
| 2526 | Y7L_P36A_R97A | 0.000 | 3.260 | 0.000 | 3.150 | 0.000 | 2.320 |
| 2527 | Y7L_P36A_R97H | 1.000 | 2.100 | 1.000 | 1.100 | 0.000 | 3.120 |
| 2528 | Y7L_P36A_V93I | 0.000 | 5.810 | 0.860 | 1.990 | 1.000 | 1.580 |
| 2529 | Y7L_P36A_V93I_R97A | 0.840 | 1.600 | 0.000 | 3.430 | 1.000 | 2.280 |
| 2530 | Y7L_P36A_V93I_R97F | 0.000 | 3.100 | 0.000 | 2.230 | 1.000 | 2.000 |
| 2531 | Y7L_P36A_V93I_R97H | 0.000 | 3.390 | 0.000 | 3.510 | 1.000 | −0.130 |
| 2532 | Y7L_P36A_V93Q | 0.000 | 3.440 | 0.000 | 3.210 | 1.000 | 0.670 |
| 2533 | Y7L_P36A_V93Q_R97A | 0.000 | 3.130 | 0.000 | 3.180 | 0.000 | 2.440 |
| 2534 | Y7L_P36A_V93R | 0.030 | 3.280 | 1.000 | 2.210 | 1.000 | 1.390 |
| 2535 | Y7L_P36H | 0.000 | 5.400 | 0.000 | 6.440 | 0.110 | 3.270 |
| 2536 | Y7L_P36H_P90F | 0.080 | 3.120 | 0.000 | 4.330 | 1.000 | 1.630 |
| 2537 | Y7L_P36H_P90F_R97H | 0.000 | 2.780 | 0.000 | 2.810 | 0.020 | 2.260 |
| 2538 | Y7L_P36H_P90F_V93I | 0.000 | 4.780 | 0.000 | 4.540 | 0.000 | 3.100 |
| 2539 | Y7L_P36H_P90W | 0.000 | 3.460 | 0.870 | 2.340 | 1.000 | 0.330 |
| 2540 | Y7L_P36H_P90W_R97A | 0.550 | 3.180 | 0.640 | 2.180 | 0.000 | 2.710 |
| 2541 | Y7L_P36H_P90W_V93I | 0.000 | 4.600 | 0.880 | 2.780 | 1.000 | 1.200 |
| 2542 | Y7L_P36H_P90Y_V93I | 0.560 | 2.680 | 0.000 | 4.900 | 1.000 | 2.640 |
| 2543 | Y7L_P36H_V93I | 0.000 | 7.140 | 0.000 | 6.380 | 0.000 | 5.750 |
| 2544 | Y7L_P36H_V93I_R97F | 0.590 | 2.050 | 0.000 | 3.060 | 1.000 | −0.660 |
| 2545 | Y7L_P36R | 0.000 | 5.850 | 0.000 | 4.760 | 1.000 | 1.940 |
| 2546 | Y7L_P36R_R97A | 0.000 | 4.140 | 0.000 | 3.980 | 0.530 | 2.710 |
| 2547 | Y7L_P36S | 0.000 | 4.860 | 0.000 | 4.400 | 0.000 | 4.280 |
| 2548 | Y7L_P36S_P90F_V93I | 0.000 | 6.730 | 0.010 | 5.050 | 0.040 | 4.420 |
| 2549 | Y7L_P36S_P90I_V93I_R97F | 0.000 | 2.800 | 1.000 | 0.340 | 0.330 | 1.860 |
| 2550 | Y7L_P36S_P90W_R97A | 0.000 | 2.250 | 0.000 | 2.230 | 0.100 | 1.520 |
| 2551 | Y7L_P36S_P90W_R97F | 0.000 | 3.160 | 1.000 | −2.150 | 1.000 | −2.070 |
| 2552 | Y7L_P36S_P90W_V93I | 0.030 | 3.530 | 0.000 | 2.470 | 0.010 | 4.530 |
| 2553 | Y7L_P36S_P90W_V93I_R97A | 1.000 | 0.100 | 0.000 | 3.260 | 1.000 | 0.460 |
| 2554 | Y7L_P36S_P90W_V93R_R97A | 0.000 | 2.170 | 1.000 | −1.600 | 1.000 | −1.500 |
| 2555 | Y7L_P36S_P90Y | 0.000 | 4.980 | 0.000 | 4.280 | 0.000 | 4.290 |
| 2556 | Y7L_P36S_P90Y_V93I | 0.000 | 4.070 | 0.000 | 4.410 | 0.170 | 2.920 |
| 2557 | Y7L_P36S_P90Y_V93I_R97A | 0.000 | 3.250 | 0.000 | 3.110 | 1.000 | 1.790 |
| 2558 | Y7L_P36S_V93I | 0.000 | 5.080 | 1.000 | 0.540 | 1.000 | 1.120 |
| 2559 | Y7L_P36S_V93I_R97W | 0.040 | 2.310 | 1.000 | 0.550 | 1.000 | 0.720 |
| 2560 | Y7L_P36S_V93Q_R97A | 0.000 | 2.100 | 1.000 | 1.110 | 1.000 | 0.710 |
| 2561 | Y7L_P36W | 0.000 | 6.720 | 0.000 | 3.470 | 0.000 | 6.210 |
| 2562 | Y7L_P36W_P90F | 0.000 | 5.180 | 0.000 | 5.210 | 0.000 | 3.830 |
| 2563 | Y7L_P36W_P90F_R97A | 0.000 | 3.050 | 0.000 | 3.360 | 1.000 | −0.440 |
| 2564 | Y7L_P36W_P90F_V93I | 0.000 | 6.540 | 0.000 | 6.110 | 0.000 | 5.030 |
| 2565 | Y7L_P36W_P90F_V93I_R97A | 0.000 | 4.010 | 0.080 | 2.930 | 0.050 | 3.090 |
| 2566 | Y7L_P36W_P90F_V93I_R97F | 0.000 | 4.390 | 0.000 | 3.890 | 1.000 | 2.340 |
| 2567 | Y7L_P36W_P90F_V93I_R97W | 1.000 | 2.410 | 0.000 | 2.740 | 1.000 | 0.270 |
| 2568 | Y7L_P36W_P90F_V93R_R97A | 0.000 | 2.180 | 1.000 | 0.710 | 0.000 | 1.570 |
| 2569 | Y7L_P36W_P90I | 0.000 | 3.200 | 1.000 | 0.570 | 1.000 | 2.130 |
| 2570 | Y7L_P36W_P90I_R97A | 0.000 | 2.950 | 1.000 | −2.480 | 1.000 | −1.730 |
| 2571 | Y7L_P36W_P90I_V93I | 0.000 | 6.780 | 0.000 | 4.650 | 0.000 | 4.940 |
| 2572 | Y7L_P36W_P90S_V93I | 0.000 | 3.380 | 0.010 | 2.980 | 0.000 | 4.670 |
| 2573 | Y7L_P36W_P90W | 0.000 | 6.380 | 0.000 | 5.670 | 0.000 | 4.590 |
| 2574 | Y7L_P36W_P90W_R97A | 0.000 | 5.540 | 0.000 | 4.140 | 1.000 | 1.790 |
| 2575 | Y7L_P36W_P90W_R97F | 0.000 | 2.700 | 0.010 | 1.950 | 0.010 | 2.000 |
| 2576 | Y7L_P36W_P90W_V93I | 0.000 | 7.680 | 0.000 | 7.080 | 0.000 | 4.680 |
| 2577 | Y7L_P36W_P90W_V93I_R97A | 0.000 | 4.000 | 0.000 | 2.880 | 1.000 | 1.020 |
| 2578 | Y7L_P36W_P90W_V93I_R97F | 0.000 | 4.840 | 0.000 | 3.730 | 0.000 | 3.510 |
| 2579 | Y7L_P36W_P90W_V93I_R97H | 0.160 | 2.500 | 0.000 | 3.820 | 1.000 | 1.830 |
| 2580 | Y7L_P36W_P90W_V93I_R97W | 1.000 | 2.620 | 0.000 | 4.300 | 0.000 | 3.970 |
| 2581 | Y7L_P36W_P90Y | 0.000 | 5.580 | 0.000 | 3.840 | 0.330 | 3.190 |
| 2582 | Y7L_P36W_P90Y_R97A | 0.030 | 2.820 | 1.000 | 1.740 | 1.000 | 1.150 |
| 2583 | Y7L_P36W_P90Y_R97F | 0.000 | 2.290 | 0.000 | 1.680 | 1.000 | 1.190 |
| 2584 | Y7L_P36W_P90Y_R97H | 0.070 | 2.560 | 0.010 | 2.560 | 1.000 | 1.760 |
| 2585 | Y7L_P36W_P90Y_R97W | 1.000 | 0.310 | 0.000 | 2.220 | 1.000 | −2.310 |
| 2586 | Y7L_P36W_P90Y_V93I | 0.000 | 5.110 | 0.000 | 4.040 | 0.070 | 2.170 |
| 2587 | Y7L_P36W_P90Y_V93I_R97F | 0.000 | 3.530 | 0.000 | 3.430 | 1.000 | 0.280 |
| 2588 | Y7L_P36W_P90Y_V93I_R97H | 0.000 | 4.700 | 1.000 | 1.440 | 1.000 | 1.000 |
| 2589 | Y7L_P36W_P90Y_V93I_R97W | 0.000 | 3.660 | 1.000 | 1.900 | 1.000 | −1.070 |
| 2590 | Y7L_P36W_P90Y_V93R_R97A | 0.000 | 2.310 | 1.000 | 0.400 | 1.000 | −0.940 |
| 2591 | Y7L_P36W_R97A | 0.000 | 4.670 | 0.000 | 3.610 | 0.020 | 3.260 |
| 2592 | Y7L_P36W_R97H | 0.000 | 3.660 | 0.080 | 3.300 | 0.000 | 3.140 |
| 2593 | Y7L_P36W_V93I | 0.000 | 7.200 | 0.000 | 4.900 | 0.000 | 6.450 |
| 2594 | Y7L_P36W_V93I_R97A | 0.000 | 3.940 | 0.000 | 3.840 | 1.000 | 1.440 |
| 2595 | Y7L_P36W_V93I_R97F | 0.000 | 4.050 | 0.000 | 3.260 | 1.000 | 2.630 |
| 2596 | Y7L_P36W_V93I_R97H | 0.000 | 2.710 | 0.260 | 2.290 | 0.000 | 4.500 |
| 2597 | Y7L_P36W_V93K | 0.000 | 2.420 | 1.000 | 0.970 | 1.000 | −0.470 |
| 2598 | Y7L_P36W_V93Q | 0.000 | 4.160 | 0.000 | 3.550 | 1.000 | 1.130 |
| 2599 | Y7L_P36W_V93R | 0.000 | 5.420 | 0.000 | 3.800 | 1.000 | 1.790 |
| 2600 | Y7L_P36W_V93R_R97A | 0.000 | 5.810 | 0.000 | 5.130 | 1.000 | 2.180 |
| 2601 | Y7L_P36W_V93R_R97F | 0.000 | 4.720 | 1.000 | −0.570 | 1.000 | 0.000 |
| 2602 | Y7L_P90F | 0.000 | 6.900 | 0.000 | 6.500 | 0.000 | 6.230 |
| 2603 | Y7L_P90F_R97A | 0.000 | 4.080 | 0.330 | 3.630 | 1.000 | 2.700 |
| 2604 | Y7L_P90F_R97F | 0.160 | 1.980 | 0.010 | 3.510 | 1.000 | 1.260 |
| 2605 | Y7L_P90F_R97H | 0.300 | 2.440 | 0.000 | 3.950 | 1.000 | 1.760 |
| 2606 | Y7L_P90F_R97W | 0.020 | 3.300 | 0.010 | 2.980 | 1.000 | 2.770 |
| 2607 | Y7L_P90F_V93E_R97F | 0.010 | 2.060 | 1.000 | −0.570 | 1.000 | −0.620 |
| 2608 | Y7L_P90F_V93I | 0.000 | 7.420 | 0.000 | 6.620 | 0.000 | 7.100 |
| 2609 | Y7L_P90F_V93I_R97A | 0.000 | 7.220 | 0.000 | 6.950 | 0.000 | 6.220 |
| 2610 | Y7L_P90F_V93I_R97F | 0.000 | 4.070 | 0.000 | 4.200 | 0.010 | 3.910 |
| 2611 | Y7L_P90F_V93I_R97H | 0.270 | 2.710 | 0.000 | 3.950 | 1.000 | 1.880 |
| 2612 | Y7L_P90F_V93I_R97W | 0.000 | 2.820 | 0.000 | 4.480 | 1.000 | 0.450 |
| 2613 | Y7L_P90F_V93K | 0.000 | 2.730 | 1.000 | 1.640 | 1.000 | 0.200 |
| 2614 | Y7L_P90F_V93K_R97A | 0.000 | 2.250 | 1.000 | 1.160 | 1.000 | 0.650 |
| 2615 | Y7L_P90F_V93K_R97F | 0.000 | 3.270 | 1.000 | −0.820 | 1.000 | −0.280 |
| 2616 | Y7L_P90F_V93K_R97F_R119Q | 0.470 | 2.510 | 1.000 | 1.470 | 0.000 | 2.350 |
| 2617 | Y7L_P90F_V93Q | 0.000 | 4.250 | 0.000 | 4.760 | 1.000 | 2.440 |
| 2618 | Y7L_P90F_V93Q_R97H | 0.000 | 2.660 | 0.020 | 2.470 | 0.510 | 2.020 |
| 2619 | Y7L_P90F_V93R | 0.000 | 3.530 | 0.460 | 2.660 | 1.000 | 0.440 |
| 2620 | Y7L_P90F_V93R_R97A | 0.000 | 4.390 | 0.010 | 3.450 | 1.000 | 1.920 |
| 2621 | Y7L_P90I | 0.000 | 6.500 | 0.000 | 3.950 | 0.000 | 5.140 |
| 2622 | Y7L_P90I_R97F | 0.000 | 4.140 | 1.000 | 2.660 | 1.000 | 1.850 |
| 2623 | Y7L_P90I_R97H | 0.150 | 3.740 | 0.010 | 3.780 | 1.000 | 0.740 |
| 2624 | Y7L_P90I_R97W | 0.000 | 3.190 | 0.000 | 3.270 | 0.000 | 2.690 |
| 2625 | Y7L_P90I_V93I | 0.000 | 5.630 | 0.920 | 2.820 | 0.100 | 2.680 |
| 2626 | Y7L_P90I_V93I_R97H | 0.000 | 4.500 | 0.000 | 3.830 | 1.000 | 1.350 |
| 2627 | Y7L_P90I_V93I_R97W | 0.020 | 4.200 | 0.010 | 3.940 | 1.000 | 1.660 |
| 2628 | Y7L_P90I_V93K_R97F | 1.000 | 1.900 | 0.030 | 2.350 | 1.000 | −3.020 |
| 2629 | Y7L_P90I_V93Q | 0.000 | 3.510 | 0.010 | 4.270 | 1.000 | 1.010 |
| 2630 | Y7L_P90I_V93R | 0.000 | 6.370 | 0.000 | 5.170 | 0.000 | 4.170 |
| 2631 | Y7L_P90S | 0.000 | 4.900 | 1.000 | 1.140 | 1.000 | −1.820 |
| 2632 | Y7L_P90S_V93I_R97H | 0.000 | 3.240 | 1.000 | −0.200 | 1.000 | −0.270 |
| 2633 | Y7L_P90S_V93R_R97H | 0.000 | 2.460 | 1.000 | 1.240 | 1.000 | 1.970 |
| 2634 | Y7L_P90W_R97A | 0.000 | 3.540 | 0.000 | 5.100 | 0.650 | 3.430 |
| 2635 | Y7L_P90W_R97F | 0.000 | 4.220 | 0.000 | 3.930 | 0.500 | 2.470 |
| 2636 | Y7L_P90W_R97H | 0.000 | 5.100 | 0.000 | 4.030 | 0.000 | 3.750 |
| 2637 | Y7L_P90W_R97W | 0.000 | 4.910 | 0.000 | 3.530 | 1.000 | 1.260 |
| 2638 | Y7L_P90W_V93I | 0.000 | 7.450 | 0.000 | 7.970 | 0.000 | 7.050 |
| 2639 | Y7L_P90W_V93I_R97A | 0.000 | 5.830 | 0.000 | 4.810 | 1.000 | 1.960 |
| 2640 | Y7L_P90W_V93I_R97F | 0.000 | 5.320 | 0.000 | 5.370 | 1.000 | 1.590 |
| 2641 | Y7L_P90W_V93I_R97H | 0.020 | 4.260 | 0.000 | 4.310 | 0.330 | 2.930 |
| 2642 | Y7L_P90W_V93I_R97W | 0.160 | 3.640 | 0.000 | 5.260 | 1.000 | 1.560 |
| 2643 | Y7L_P90W_V93Q | 0.010 | 3.860 | 0.000 | 5.010 | 0.010 | 4.050 |
| 2644 | Y7L_P90W_V93R | 0.000 | 6.630 | 0.000 | 4.980 | 1.000 | 1.950 |
| 2645 | Y7L_P90Y | 0.000 | 6.270 | 0.000 | 4.980 | 0.000 | 4.680 |
| 2646 | Y7L_P90Y_R97F | 0.390 | 2.230 | 0.000 | 3.680 | 1.000 | 1.070 |
| 2647 | Y7L_P90Y_R97H | 1.000 | 2.050 | 0.000 | 3.230 | 1.000 | 0.010 |
| 2648 | Y7L_P90Y_R97W | 1.000 | 1.910 | 0.010 | 2.580 | 0.920 | 2.010 |
| 2649 | Y7L_P90Y_V93I | 0.000 | 6.690 | 0.000 | 4.960 | 0.000 | 4.970 |
| 2650 | Y7L_P90Y_V93I_R97A | 0.000 | 4.860 | 0.000 | 5.400 | 0.100 | 3.600 |
| 2651 | Y7L_P90Y_V93I_R97F | 0.000 | 4.610 | 0.000 | 4.540 | 1.000 | 1.580 |
| 2652 | Y7L_P90Y_V93I_R97H | 0.000 | 4.300 | 0.000 | 4.770 | 0.000 | 4.180 |
| 2653 | Y7L_P90Y_V93R_R97A | 0.000 | 3.250 | 1.000 | 1.880 | 1.000 | 0.300 |
| 2654 | Y7L_R97F | 0.000 | 3.200 | 0.010 | 3.600 | 0.000 | 1.740 |
| 2655 | Y7L_R97H | 0.020 | 2.540 | 1.000 | 2.410 | 0.730 | 2.760 |
| 2656 | Y7L_R97W | 0.000 | 6.330 | 0.000 | 5.890 | 0.000 | 5.770 |
| 2657 | Y7L_V93I | 0.000 | 4.310 | 0.000 | 2.830 | 0.000 | 2.770 |
| 2658 | Y7L_V93I_R97F | 0.000 | 4.010 | 0.000 | 3.340 | 1.000 | 1.320 |
| 2659 | Y7L_V93I_R97H | 0.180 | 3.480 | 1.000 | 1.430 | 0.000 | 4.020 |
| 2660 | Y7L_V93I_R97W | 0.010 | 4.290 | 0.010 | 3.140 | 1.000 | 1.120 |
| 2661 | Y7L_V93K | 0.020 | 2.970 | 0.000 | 4.380 | 1.000 | 0.550 |
| 2662 | Y7L_V93K_R97H | 1.000 | 1.150 | 0.010 | 2.670 | 1.000 | 1.010 |
| 2663 | Y7L_V93Q | 0.000 | 5.630 | 0.000 | 5.240 | 1.000 | 1.540 |
| 2664 | Y7L_V93Q_R97A | 0.000 | 2.170 | 1.000 | 1.560 | 1.000 | 0.910 |
| 2665 | Y7L_V93Q_R97H | 1.000 | 1.120 | 0.000 | 2.590 | 1.000 | −0.200 |
| 2666 | Y7L_V93Q_R97W | 0.000 | 2.480 | 0.010 | 2.870 | 1.000 | −0.710 |
| 2667 | Y7L_V93R | 0.000 | 6.220 | 0.000 | 6.630 | 0.010 | 3.800 |
| 2668 | Y7L_V93R_R97A | 0.000 | 4.860 | 0.000 | 5.330 | 1.000 | 1.530 |
| 2669 | Y7L_V93R_R97F | 0.680 | 2.820 | 0.000 | 4.230 | 1.000 | 0.030 |
| 2670 | Y7L_V93R_R97W | 0.000 | 3.560 | 1.000 | 2.570 | 1.000 | 0.240 |
| 42 | Y7M | 0.000 | 3.947 | 0.000 | 1.876 | 0.008 | 1.704 |
| 2671 | Y7M_I16F | 0.000 | 5.150 | 0.010 | 1.970 | 0.000 | 3.070 |
| 2672 | Y7M_I16F_P36A | 0.000 | 3.880 | 1.000 | 1.090 | 1.000 | 1.450 |
| 2673 | Y7M_I16F_P36A_P90F | 0.030 | 3.700 | 1.000 | −0.760 | 1.000 | 0.880 |
| 2674 | Y7M_I16F_P36A_P90F_V93I | 0.010 | 2.810 | 1.000 | 1.830 | 1.000 | 1.390 |
| 2675 | Y7M_I16F_P36A_P90W_V93I | 0.000 | 3.410 | 0.010 | 3.430 | 0.000 | 2.560 |
| 2676 | Y7M_I16F_P36A_P90Y_V93I_R97A | 0.010 | 2.140 | 1.000 | −0.580 | 1.000 | −0.560 |
| 2677 | Y7M_I16F_P36A_R97A | 1.000 | 1.070 | 0.000 | 3.510 | 1.000 | 1.270 |
| 2678 | Y7M_I16F_P36A_V93I | 0.000 | 5.910 | 1.000 | −0.450 | 1.000 | −0.870 |
| 2679 | Y7M_I16F_P36A_V93I_R97A | 0.000 | 4.480 | 0.000 | 2.720 | 0.000 | 3.360 |
| 2680 | Y7M_I16F_P36A_V93I_R97F | 1.000 | 0.330 | 1.000 | 1.200 | 0.010 | 3.250 |
| 2681 | Y7M_I16F_P36A_V93Q | 0.000 | 3.650 | 0.000 | 3.280 | 0.000 | 3.290 |
| 2682 | Y7M_I16F_P36H | 1.000 | 1.550 | 0.000 | 3.480 | 1.000 | 1.350 |
| 2683 | Y7M_I16F_P36H_V93I | 0.130 | 3.070 | 0.020 | 2.850 | 0.490 | 2.710 |
| 2684 | Y7M_I16F_P36S_V93I | 0.000 | 5.170 | 1.000 | −0.500 | 1.000 | 1.270 |
| 2685 | Y7M_I16F_P36W | 0.320 | 2.330 | 0.000 | 2.170 | 1.000 | 2.530 |
| 2686 | Y7M_I16F_P36W_R97A | 1.000 | 4.340 | 0.520 | 1.560 | 0.000 | 2.010 |
| 2687 | Y7M_I16F_P90F | 0.000 | 6.680 | 0.000 | 6.640 | 0.000 | 5.560 |
| 2688 | Y7M_I16F_P90F_R97A | 0.000 | 5.040 | 0.000 | 4.450 | 0.010 | 3.100 |
| 2689 | Y7M_I16F_P90F_R97H | 0.000 | 3.940 | 0.000 | 3.480 | 1.000 | 2.060 |
| 2690 | Y7M_I16F_P90F_V93I | 0.000 | 8.110 | 0.000 | 7.570 | 0.000 | 7.080 |
| 2691 | Y7M_I16F_P90F_V93I_R97A | 0.000 | 5.670 | 0.000 | 4.980 | 0.120 | 3.140 |
| 2692 | Y7M_I16F_P90F_V93I_R97F | 0.000 | 4.760 | 0.000 | 5.300 | 1.000 | 2.420 |
| 2693 | Y7M_I16F_P90F_V93I_R97H | 0.000 | 4.830 | 0.000 | 2.990 | 1.000 | 2.290 |
| 2694 | Y7M_I16F_P90F_V93Q | 0.000 | 4.290 | 0.000 | 2.850 | 0.050 | 2.170 |
| 2695 | Y7M_I16F_P90F_V93Q_R97W | 0.000 | 2.540 | 1.000 | −0.960 | 1.000 | −0.280 |
| 2696 | Y7M_I16F_P90F_V93R_R97A | 0.000 | 5.290 | 0.920 | 2.130 | 1.000 | 2.160 |
| 2697 | Y7M_I16F_P90I | 0.000 | 5.780 | 0.000 | 5.710 | 0.000 | 4.630 |
| 2698 | Y7M_I16F_P90I_R97W | 0.000 | 2.140 | 0.000 | 4.820 | 0.000 | 3.180 |
| 2699 | Y7M_I16F_P90I_V93I | 0.000 | 6.800 | 0.000 | 6.800 | 0.000 | 7.560 |
| 2700 | Y7M_I16F_P90I_V93I_R97A | 0.000 | 5.010 | 0.000 | 5.800 | 0.000 | 5.050 |
| 2701 | Y7M_I16F_P90I_V93I_R97F | 0.000 | 3.810 | 0.000 | 3.200 | 1.000 | 0.750 |
| 2702 | Y7M_I16F_P90I_V93I_R97H | 0.000 | 7.520 | 0.000 | 4.170 | 0.060 | 3.970 |
| 2703 | Y7M_I16F_P90I_V93Q | 0.000 | 3.880 | 1.000 | 2.280 | 1.000 | 0.690 |
| 2704 | Y7M_I16F_P90I_V93Q_R97H | 1.000 | 1.740 | 1.000 | −0.010 | 0.000 | 2.330 |
| 2705 | Y7M_I16F_P90I_V93R | 0.000 | 3.260 | 0.000 | 2.330 | 0.010 | 1.860 |
| 2706 | Y7M_I16F_P90I_V93R_R97F | 0.000 | 2.790 | 1.000 | 1.610 | 1.000 | −2.280 |
| 2707 | Y7M_I16F_P90S_R97A | 0.000 | 4.690 | 0.000 | 4.940 | 0.000 | 5.500 |
| 2708 | Y7M_I16F_P90S_R97H | 1.000 | −1.380 | 0.000 | 2.470 | 1.000 | 0.730 |
| 2709 | Y7M_I16F_P90S_V93I | 0.000 | 4.920 | 1.000 | −1.570 | 1.000 | −1.870 |
| 2710 | Y7M_I16F_P90S_V93I_R97A | 0.000 | 3.840 | 1.000 | 0.360 | 1.000 | 1.440 |
| 2711 | Y7M_I16F_P90S_V93I_R97F | 0.000 | 2.510 | 0.100 | 1.900 | 0.000 | 2.670 |
| 2712 | Y7M_I16F_P90W | 0.000 | 5.800 | 0.000 | 5.550 | 0.000 | 6.160 |
| 2713 | Y7M_I16F_P90W_R97A | 0.000 | 2.790 | 0.000 | 4.470 | 1.000 | 2.520 |
| 2714 | Y7M_I16F_P90W_V93I | 0.000 | 6.810 | 0.000 | 5.840 | 0.000 | 6.110 |
| 2715 | Y7M_I16F_P90W_V93I_R97A | 0.000 | 4.920 | 0.000 | 3.720 | 1.000 | 2.660 |
| 2716 | Y7M_I16F_P90W_V93I_R97F | 0.000 | 3.950 | 0.260 | 2.720 | 0.190 | 1.990 |
| 2717 | Y7M_I16F_P90W_V93I_R97H | 0.000 | 3.710 | 0.000 | 3.670 | 0.400 | 2.870 |
| 2718 | Y7M_I16F_P90W_V93Q | 1.000 | 0.650 | 0.000 | 2.960 | 1.000 | 0.740 |
| 2719 | Y7M_I16F_P90W_V93R_R97A | 0.000 | 2.140 | 1.000 | 1.450 | 1.000 | −0.950 |
| 2720 | Y7M_I16F_P90W_V93R_R97W | 0.000 | 2.180 | 1.000 | −2.770 | 1.000 | −2.630 |
| 2721 | Y7M_I16F_P90Y | 0.000 | 6.270 | 0.000 | 5.540 | 0.000 | 6.120 |
| 2722 | Y7M_I16F_P90Y_R97A | 0.000 | 2.730 | 0.000 | 2.740 | 1.000 | 2.260 |
| 2723 | Y7M_I16F_P90Y_R97H | 0.000 | 2.830 | 1.000 | 0.700 | 1.000 | 1.810 |
| 2724 | Y7M_I16F_P90Y_V93I | 0.000 | 6.540 | 0.000 | 5.150 | 0.000 | 6.250 |
| 2725 | Y7M_I16F_P90Y_V93I_R97A | 0.000 | 3.580 | 0.000 | 4.580 | 0.790 | 2.260 |
| 2726 | Y7M_I16F_P90Y_V93I_R97F | 1.000 | 3.120 | 0.000 | 4.060 | 1.000 | 0.790 |
| 2727 | Y7M_I16F_P90Y_V93I_R97H | 0.000 | 4.060 | 0.000 | 3.930 | 1.000 | 2.270 |
| 2728 | Y7M_I16F_P90Y_V93I_R97W | 0.000 | 2.510 | 0.000 | 2.480 | 1.000 | 1.640 |
| 2729 | Y7M_I16F_R97A | 0.000 | 5.650 | 0.000 | 5.290 | 0.000 | 4.220 |
| 2730 | Y7M_I16F_R97F | 0.000 | 5.200 | 0.000 | 5.160 | 0.000 | 3.030 |
| 2731 | Y7M_I16F_R97H | 1.000 | 2.820 | 0.000 | 4.240 | 0.000 | 3.210 |
| 2732 | Y7M_I16F_R97W | 0.000 | 4.810 | 0.000 | 3.370 | 0.000 | 4.500 |
| 2733 | Y7M_I16F_R119Q | 0.000 | 2.750 | 1.000 | 1.540 | 0.000 | 2.450 |
| 2734 | Y7M_I16F_V93I | 0.000 | 5.860 | 0.000 | 1.870 | 0.000 | 2.220 |
| 2735 | Y7M_I16F_V93I_R97A | 0.000 | 4.930 | 0.000 | 3.710 | 0.000 | 5.440 |
| 2736 | Y7M_I16F_V93I_R97F | 0.000 | 3.660 | 0.000 | 4.140 | 0.010 | 3.980 |
| 2737 | Y7M_I16F_V93I_R97H | 0.000 | 5.510 | 0.280 | 3.440 | 0.000 | 4.400 |
| 2738 | Y7M_I16F_V93I_R97W | 0.000 | 2.570 | 0.000 | 3.480 | 0.130 | 3.070 |
| 2739 | Y7M_I16F_V93K | 0.000 | 5.040 | 1.000 | 1.200 | 1.000 | 1.550 |
| 2740 | Y7M_I16F_V93Q | 0.000 | 4.190 | 0.000 | 3.970 | 0.000 | 4.370 |
| 2741 | Y7M_I16F_V93Q_R97H | 1.000 | 0.520 | 0.000 | 2.770 | 1.000 | 0.460 |
| 2742 | Y7M_I16F_V93R | 0.000 | 4.260 | 0.000 | 3.720 | 0.000 | 3.970 |
| 2743 | Y7M_I16F_V93R_R97A | 0.000 | 4.850 | 0.000 | 4.960 | 0.020 | 3.270 |
| 2744 | Y7M_I16F_V93R_R97F | 0.000 | 2.940 | 0.030 | 2.840 | 1.000 | 0.550 |
| 2745 | Y7M_I16F_V93R_R97W | 0.000 | 2.950 | 0.010 | 1.720 | 1.000 | 0.550 |
| 2746 | Y7M_I16H_P36H_P90W | 0.000 | 2.010 | 1.000 | 1.230 | 0.000 | 2.150 |
| 2747 | Y7M_I16H_P36H_V93I | 0.000 | 4.820 | 0.070 | 3.430 | 0.000 | 5.180 |
| 2748 | Y7M_I16H_P36H_V93R | 0.000 | 5.070 | 0.010 | 3.880 | 0.000 | 3.540 |
| 2749 | Y7M_I16H_P90W_V93I | 0.000 | 2.740 | 0.000 | 1.920 | 0.000 | 2.660 |
| 2750 | Y7M_I16L | 0.000 | 10.550 | 0.000 | 9.580 | 0.000 | 9.530 |
| 2751 | Y7M_I16L_P36A | 0.000 | 6.320 | 0.000 | 2.150 | 1.000 | 1.260 |
| 2752 | Y7M_I16L_P36A_P90F | 0.000 | 3.570 | 1.000 | 0.770 | 1.000 | 1.010 |
| 2753 | Y7M_I16L_P36A_P90F_V93I | 0.630 | 3.040 | 0.000 | 2.780 | 1.000 | 2.420 |
| 2754 | Y7M_I16L_P36A_P90F_V93I_R97H | 0.000 | 2.900 | 0.000 | 3.160 | 0.000 | 2.900 |
| 2755 | Y7M_I16L_P36A_P90I_R97F | 0.000 | 3.160 | 0.010 | 2.320 | 0.000 | 2.300 |
| 2756 | Y7M_I16L_P36A_P90I_V93I_R97H | 0.000 | 2.520 | 1.000 | 1.100 | 0.000 | 2.060 |
| 2757 | Y7M_I16L_P36A_P90S_V93I_R97H | 0.880 | 2.480 | 1.000 | 0.150 | 0.000 | 2.130 |
| 2758 | Y7M_I16L_P36A_P90W | 0.030 | 2.810 | 1.000 | 0.920 | 1.000 | 0.260 |
| 2759 | Y7M_I16L_P36A_P90W_V93I | 0.000 | 3.350 | 1.000 | 2.040 | 0.000 | 3.670 |
| 2760 | Y7M_I16L_P36A_P90W_V93I_R97A | 0.000 | 3.520 | 1.000 | 2.810 | 0.150 | 2.700 |
| 2761 | Y7M_I16L_P36A_P90Y_V93I | 0.000 | 4.030 | 0.000 | 4.780 | 1.000 | 1.830 |
| 2762 | Y7M_I16L_P36A_R97F | 0.000 | 5.840 | 1.000 | 0.290 | 1.000 | 1.850 |
| 2763 | Y7M_I16L_P36A_R97H | 0.000 | 4.300 | 0.260 | 3.540 | 0.130 | 3.700 |
| 2764 | Y7M_I16L_P36A_R97W | 0.000 | 4.200 | 1.000 | 2.480 | 1.000 | 2.380 |
| 2765 | Y7M_I16L_P36A_V93I | 0.000 | 7.130 | 0.000 | 2.680 | 1.000 | 1.520 |
| 2766 | Y7M_I16L_P36A_V93I_R97A | 0.000 | 4.440 | 0.000 | 5.380 | 0.000 | 3.650 |
| 2767 | Y7M_I16L_P36A_V93I_R97F | 0.000 | 5.480 | 0.000 | 4.630 | 1.000 | 2.310 |
| 2768 | Y7M_I16L_P36A_V93I_R97H | 0.000 | 4.870 | 0.000 | 5.340 | 0.000 | 4.770 |
| 2769 | Y7M_I16L_P36A_V93Q | 0.000 | 2.800 | 1.000 | 2.130 | 1.000 | 0.640 |
| 2770 | Y7M_I16L_P36A_V93Q_R97A | 0.000 | 3.650 | 1.000 | 2.170 | 1.000 | 0.640 |
| 2771 | Y7M_I16L_P36A_V93R | 0.000 | 4.790 | 0.000 | 4.710 | 1.000 | 1.840 |
| 2772 | Y7M_I16L_P36A_V93R_R97A | 0.000 | 5.280 | 0.000 | 4.600 | 1.000 | 2.980 |
| 2773 | Y7M_I16L_P36H | 0.000 | 7.550 | 0.000 | 7.350 | 0.000 | 5.450 |
| 2774 | Y7M_I16L_P36H_P90F_R97A | 0.000 | 2.550 | 1.000 | 1.180 | 1.000 | −0.110 |
| 2775 | Y7M_I16L_P36H_P90F_V93I | 1.000 | 1.030 | 0.030 | 2.410 | 1.000 | 0.950 |
| 2776 | Y7M_I16L_P36H_R97W | 1.000 | 0.640 | 0.000 | 3.100 | 1.000 | 0.830 |
| 2777 | Y7M_I16L_P36H_V93I | 0.000 | 7.960 | 0.000 | 7.280 | 0.000 | 5.880 |
| 2778 | Y7M_I16L_P36H_V93Q | 0.010 | 2.570 | 1.000 | 1.310 | 1.000 | 0.300 |
| 2779 | Y7M_I16L_P36H_V93Q_R97F | 0.000 | 3.020 | 1.000 | 0.750 | 0.440 | 1.620 |
| 2780 | Y7M_I16L_P36R | 1.000 | 1.640 | 0.000 | 3.420 | 1.000 | −0.120 |
| 2781 | Y7M_I16L_P36S | 0.000 | 4.720 | 1.000 | −0.060 | 1.000 | −0.540 |
| 2782 | Y7M_I16L_P36S_P90F_V93I | 0.000 | 4.580 | 0.000 | 3.320 | 0.000 | 2.960 |
| 2783 | Y7M_I16L_P36S_P90W_V93I | 0.000 | 3.050 | 1.000 | 1.090 | 1.000 | −0.270 |
| 2784 | Y7M_I16L_P36S_P90Y_V93I | 0.000 | 4.180 | 0.000 | 3.150 | 0.000 | 2.800 |
| 2785 | Y7M_I16L_P36S_R97A | 0.000 | 3.760 | 0.000 | 3.170 | 1.000 | 0.470 |
| 2786 | Y7M_I16L_P36S_V93I | 0.000 | 7.570 | 1.000 | 0.280 | 1.000 | 0.300 |
| 2787 | Y7M_I16L_P36S_V93I_R97A | 1.000 | 2.520 | 0.000 | 3.250 | 1.000 | 2.180 |
| 2788 | Y7M_I16L_P36S_V93I_R97H | 0.020 | 3.440 | 0.000 | 2.970 | 1.000 | −0.350 |
| 2789 | Y7M_I16L_P36W | 0.000 | 8.140 | 0.000 | 4.070 | 0.000 | 3.620 |
| 2790 | Y7M_I16L_P36W_P90F | 0.000 | 2.750 | 0.000 | 5.290 | 0.100 | 2.460 |
| 2791 | Y7M_I16L_P36W_P90F_V93I | 0.000 | 5.810 | 0.000 | 5.650 | 0.000 | 4.150 |
| 2792 | Y7M_I16L_P36W_P90F_V93I_R97A | 0.000 | 4.170 | 0.000 | 4.000 | 0.000 | 3.570 |
| 2793 | Y7M_I16L_P36W_P90I_V93I | 0.000 | 4.970 | 1.000 | 1.190 | 1.000 | 2.140 |
| 2794 | Y7M_I16L_P36W_P90W | 0.000 | 3.680 | 1.000 | 2.250 | 0.250 | 2.480 |
| 2795 | Y7M_I16L_P36W_P90W_R97A | 0.000 | 4.130 | 0.590 | 2.870 | 1.000 | 0.950 |
| 2796 | Y7M_I16L_P36W_P90W_R97H | 0.010 | 3.910 | 0.000 | 2.540 | 0.100 | 1.570 |
| 2797 | Y7M_I16L_P36W_P90W_V93I | 0.000 | 5.710 | 0.000 | 5.240 | 0.000 | 4.810 |
| 2798 | Y7M_I16L_P36W_P90Y | 0.000 | 4.280 | 1.000 | 2.340 | 0.360 | 2.180 |
| 2799 | Y7M_I16L_P36W_P90Y_V93I | 0.000 | 4.950 | 0.000 | 5.230 | 0.000 | 4.020 |
| 2800 | Y7M_I16L_P36W_P90Y_V93I_R97A | 0.000 | 3.620 | 0.000 | 2.730 | 1.000 | 1.970 |
| 2801 | Y7M_I16L_P36W_P90Y_V93I_R97H | 0.010 | 2.140 | 0.000 | 2.150 | 1.000 | 0.700 |
| 2802 | Y7M_I16L_P36W_R97A | 0.000 | 5.820 | 0.000 | 6.230 | 0.500 | 3.310 |
| 2803 | Y7M_I16L_P36W_R97F | 0.030 | 2.540 | 0.540 | 2.930 | 0.010 | 3.270 |
| 2804 | Y7M_I16L_P36W_R97H | 0.000 | 5.160 | 0.000 | 4.430 | 0.000 | 3.760 |
| 2805 | Y7M_I16L_P36W_R97W | 0.000 | 3.800 | 0.000 | 3.340 | 1.000 | 0.300 |
| 2806 | Y7M_I16L_P36W_V93I | 0.000 | 10.230 | 0.000 | 3.810 | 0.000 | 3.560 |
| 2807 | Y7M_I16L_P36W_V93I_R97A | 0.000 | 5.810 | 0.000 | 5.090 | 0.000 | 4.690 |
| 2808 | Y7M_I16L_P36W_V93I_R97F | 0.000 | 4.130 | 0.000 | 4.440 | 0.000 | 4.320 |
| 2809 | Y7M_I16L_P36W_V93I_R97H | 0.000 | 5.050 | 0.000 | 4.370 | 0.000 | 4.140 |
| 2810 | Y7M_I16L_P36W_V93I_R97W | 0.470 | 3.800 | 0.000 | 4.230 | 0.000 | 3.760 |
| 2811 | Y7M_I16L_P36W_V93Q | 0.000 | 3.360 | 1.000 | 2.710 | 1.000 | 0.760 |
| 2812 | Y7M_I16L_P36W_V93Q_R97A | 0.020 | 2.600 | 1.000 | −0.060 | 1.000 | −0.440 |
| 2813 | Y7M_I16L_P36W_V93R_R97A | 0.000 | 4.740 | 0.000 | 5.880 | 0.010 | 3.670 |
| 2814 | Y7M_I16L_P90F | 0.000 | 6.170 | 0.000 | 4.460 | 0.000 | 6.280 |
| 2815 | Y7M_I16L_P90F_R97A | 0.000 | 5.630 | 0.000 | 4.620 | 1.000 | 1.750 |
| 2816 | Y7M_I16L_P90F_R97F | 0.000 | 3.980 | 0.000 | 2.910 | 0.110 | 2.680 |
| 2817 | Y7M_I16L_P90F_R97H | 0.000 | 4.710 | 0.620 | 2.810 | 1.000 | 2.350 |
| 2818 | Y7M_I16L_P90F_R119Q | 0.000 | 4.350 | 0.000 | 3.880 | 0.000 | 3.660 |
| 2819 | Y7M_I16L_P90F_V93I | 0.000 | 8.790 | 0.000 | 7.630 | 0.000 | 7.900 |
| 2820 | Y7M_I16L_P90F_V93I_R97A | 0.010 | 4.470 | 0.000 | 5.030 | 0.050 | 2.580 |
| 2821 | Y7M_I16L_P90F_V93I_R97F | 0.170 | 3.490 | 0.000 | 5.070 | 0.250 | 2.250 |
| 2822 | Y7M_I16L_P90F_V93I_R97H | 0.000 | 3.540 | 0.000 | 3.520 | 0.000 | 1.830 |
| 2823 | Y7M_I16L_P90F_V93K_R119Q | 0.000 | 3.250 | 0.000 | 2.470 | 0.000 | 2.690 |
| 2824 | Y7M_I16L_P90F_V93Q | 0.010 | 4.400 | 0.020 | 2.450 | 1.000 | −0.570 |
| 2825 | Y7M_I16L_P90F_V93Q_R97A | 0.000 | 2.730 | 0.090 | 2.850 | 1.000 | −0.500 |
| 2826 | Y7M_I16L_P90F_V93R_R97W | 0.000 | 3.960 | 0.000 | 3.100 | 0.000 | 2.930 |
| 2827 | Y7M_I16L_P90I | 0.000 | 7.430 | 0.000 | 5.230 | 0.000 | 5.810 |
| 2828 | Y7M_I16L_P90I_R97F | 0.000 | 4.130 | 1.000 | 2.190 | 1.000 | 1.210 |
| 2829 | Y7M_I16L_P90I_V93I | 0.000 | 8.250 | 0.000 | 4.170 | 0.000 | 5.530 |
| 2830 | Y7M_I16L_P90I_V93I_R97A | 0.000 | 4.540 | 0.010 | 3.280 | 0.080 | 3.480 |
| 2831 | Y7M_I16L_P90I_V93I_R97H | 0.030 | 3.260 | 0.000 | 3.090 | 1.000 | 1.970 |
| 2832 | Y7M_116T_ P90T_V93Q | 0 000 | 3 490 | 0.000 | 3 330 | 1 000 | 1.760 |
| 2833 | Y7M_I16L_P90I_V93R | 0.000 | 4.380 | 0.230 | 1.910 | 1.000 | 0.340 |
| 2834 | Y7M_I16L_P90S_R97A | 0.000 | 4.400 | 0.010 | 3.290 | 0.000 | 4.250 |
| 2835 | Y7M_I16L_P90S_V93I | 0.000 | 5.190 | 1.000 | −0.620 | 1.000 | −0.640 |
| 2836 | Y7M_I16L_P90S_V93I_R97A | 1.000 | 2.080 | 0.940 | 2.800 | 0.020 | 3.640 |
| 2837 | Y7M_I16L_P90W | 0.000 | 8.220 | 0.000 | 7.820 | 0.000 | 8.320 |
| 2838 | Y7M_I16L_P90W_R97A | 0.000 | 5.910 | 0.000 | 5.550 | 0.000 | 4.140 |
| 2839 | Y7M_I16L_P90W_R97F | 0.000 | 4.550 | 0.000 | 4.410 | 0.440 | 2.430 |
| 2840 | Y7M_I16L_P90W_R97H | 0.030 | 4.410 | 0.000 | 3.320 | 0.140 | 3.000 |
| 2841 | Y7M_I16L_P90W_V93I | 0.000 | 9.040 | 0.000 | 9.200 | 0.000 | 8.390 |
| 2842 | Y7M_I16L_P90W_V93I_R97A | 0.000 | 6.220 | 0.000 | 4.780 | 0.000 | 4.960 |
| 2843 | Y7M_I16L_P90W_V93I_R97F | 0.040 | 4.430 | 0.010 | 4.770 | 0.000 | 3.830 |
| 2844 | Y7M_I16L_P90W_V93I_R97H | 0.000 | 5.780 | 0.000 | 5.010 | 0.000 | 5.520 |
| 2845 | Y7M_I16L_P90W_V93I_R97W | 0.000 | 5.000 | 0.000 | 3.760 | 0.040 | 3.220 |
| 2846 | Y7M_I16L_P90W_V93Q | 0.000 | 5.630 | 0.000 | 5.630 | 0.030 | 3.610 |
| 2847 | Y7M_I16L_P90W_V93Q_R97A | 0.000 | 4.730 | 0.000 | 4.560 | 1.000 | 2.620 |
| 2848 | Y7M_I16L_P90W_V93Q_R97W | 0.000 | 2.540 | 0.000 | 3.090 | 0.000 | 2.700 |
| 2849 | Y7M_I16L_P90W_V93R_R97A | 0.000 | 4.420 | 0.010 | 2.780 | 1.000 | 0.500 |
| 2850 | Y7M_I16L_P90W_V93R_R97F | 0.000 | 2.300 | 1.000 | 2.080 | 1.000 | 0.900 |
| 2851 | Y7M_I16L_P90Y | 0.000 | 4.620 | 0.000 | 4.480 | 0.000 | 5.730 |
| 2852 | Y7M_I16L_P90Y_R97A | 0.000 | 5.010 | 0.000 | 6.370 | 1.000 | 2.030 |
| 2853 | Y7M_I16L_P90Y_R97F | 0.000 | 3.200 | 0.000 | 1.860 | 0.070 | 2.160 |
| 2854 | Y7M_I16L_P90Y_R97H | 0.340 | 2.510 | 0.000 | 3.880 | 1.000 | 1.300 |
| 2855 | Y7M_I16L_P90Y_V93I | 0.000 | 7.120 | 0.000 | 7.710 | 0.000 | 7.920 |
| 2856 | Y7M_I16L_P90Y_V93I_R97A | 0.000 | 4.570 | 0.020 | 4.360 | 0.000 | 4.190 |
| 2857 | Y7M_I16L_P90Y_V93I_R97F | 0.000 | 5.960 | 0.000 | 4.910 | 0.040 | 3.480 |
| 2858 | Y7M_I16L_P90Y_V93I_R97H | 0.000 | 6.210 | 0.000 | 3.200 | 0.000 | 2.630 |
| 2859 | Y7M_I16L_P90Y_V93I_R97W | 0.060 | 3.400 | 0.000 | 3.040 | 1.000 | 0.240 |
| 2860 | Y7M_I16L_P90Y_V93Q | 0.000 | 4.740 | 0.010 | 3.330 | 1.000 | 2.440 |
| 2861 | Y7M_I16L_P90Y_V93Q_R97A | 0.000 | 4.170 | 1.000 | 3.290 | 1.000 | −1.050 |
| 2862 | Y7M_I16L_P90Y_V93R_R97W | 0.000 | 2.320 | 1.000 | −0.590 | 1.000 | −1.770 |
| 2863 | Y7M_I16L_R97A | 0.000 | 6.970 | 0.000 | 5.930 | 0.000 | 6.830 |
| 2864 | Y7M_I16L_R97A_R119M | 1.000 | −0.730 | 0.000 | 2.530 | 0.930 | 1.720 |
| 2865 | Y7M_I16L_R97F | 0.000 | 6.030 | 0.000 | 5.450 | 0.000 | 5.470 |
| 2866 | Y7M_I16L_R97H | 0.000 | 5.770 | 0.000 | 5.840 | 0.290 | 2.680 |
| 2867 | Y7M_I16L_R97W | 0.000 | 4.960 | 0.000 | 4.550 | 0.000 | 5.330 |
| 2868 | Y7M_I16L_V93E | 0.000 | 4.740 | 0.920 | 2.640 | 0.060 | 3.150 |
| 2869 | Y7M_I16L_V93I | 0.000 | 5.760 | 0.000 | 1.920 | 0.070 | 2.230 |
| 2870 | Y7M_I16L_V93I_R97A | 0.000 | 6.920 | 0.000 | 5.700 | 0.000 | 6.570 |
| 2871 | Y7M_I16L_V93I_R97F | 0.000 | 6.700 | 0.000 | 6.940 | 0.000 | 5.150 |
| 2872 | Y7M_I16L_V93I_R97H | 0.000 | 6.070 | 0.000 | 5.160 | 0.000 | 5.300 |
| 2873 | Y7M_I16L_V93I_R97P | 0.000 | 3.410 | 1.000 | 3.660 | 0.000 | 3.730 |
| 2874 | Y7M_I16L_V93I_R97W | 0.000 | 5.260 | 0.000 | 5.300 | 0.000 | 4.170 |
| 2875 | Y7M_I16L_V93K | 0.000 | 6.380 | 0.000 | 6.020 | 0.000 | 4.390 |
| 2876 | Y7M_I16L_V93K_R97A | 0.030 | 4.420 | 0.000 | 4.530 | 1.000 | −0.680 |
| 2877 | Y7M_I16L_V93K_R97F | 0.000 | 3.070 | 0.070 | 2.640 | 1.000 | 1.980 |
| 2878 | Y7M_I16L_V93Q | 0.000 | 8.400 | 0.000 | 7.920 | 0.000 | 7.190 |
| 2879 | Y7M_I16L_V93Q_R97A | 0.000 | 5.320 | 0.000 | 5.340 | 0.000 | 4.910 |
| 2880 | Y7M_I16L_V93Q_R97H | 0.060 | 4.310 | 0.000 | 4.040 | 1.000 | 1.610 |
| 2881 | Y7M_I16L_V93Q_R97W | 0.000 | 4.870 | 0.000 | 4.170 | 1.000 | 2.660 |
| 2882 | Y7M_I16L_V93R | 0.000 | 7.630 | 0.000 | 7.070 | 0.000 | 5.130 |
| 2883 | Y7M_I16L_V93R_R97A | 0.000 | 5.210 | 0.000 | 5.170 | 0.000 | 4.020 |
| 2884 | Y7M_I16L_V93R_R97A_R119Q | 0.000 | 4.040 | 0.000 | 3.410 | 0.000 | 3.490 |
| 2885 | Y7M_I16L_V93R_R97F | 0.140 | 4.190 | 0.000 | 6.080 | 0.050 | 2.760 |
| 2886 | Y7M_I16L_V93R_R97H | 0.000 | 5.810 | 0.000 | 6.010 | 0.000 | 5.250 |
| 2887 | Y7M_I16L_V93R_R97W | 0.140 | 3.490 | 0.000 | 2.120 | 0.000 | 1.840 |
| 2888 | Y7M_I16M | 0.000 | 4.590 | 0.000 | 2.220 | 0.000 | 2.380 |
| 2889 | Y7M_I16M_P36A | 0.000 | 4.900 | 1.000 | 0.700 | 1.000 | 1.170 |
| 2890 | Y7M_I16M_P36A_P90W_V93I | 0.000 | 2.840 | 0.000 | 3.640 | 1.000 | 0.480 |
| 2891 | Y7M_I16M_P36A_P90Y_V93I | 0.000 | 2.870 | 0.110 | 2.290 | 1.000 | −0.740 |
| 2892 | Y7M_I16M_P36A_R97H | 1.000 | 1.500 | 0.010 | 3.390 | 1.000 | 0.620 |
| 2893 | Y7M_I16M_P36A_V93I | 0.000 | 5.660 | 1.000 | −0.490 | 1.000 | 0.680 |
| 2894 | Y7M_I16M_P36A_V93I_R97A | 0.000 | 3.830 | 0.000 | 2.810 | 1.000 | 1.400 |
| 2895 | Y7M_I16M_P36A_V93I_R97F | 0.000 | 3.830 | 0.000 | 3.770 | 1.000 | 1.970 |
| 2896 | Y7M_I16M_P36A_V93R | 0.000 | 5.210 | 0.230 | 1.960 | 1.000 | 0.940 |
| 2897 | Y7M_I16M_P36A_V93R_R97A | 0.000 | 3.860 | 0.060 | 2.820 | 1.000 | 1.600 |
| 2898 | Y7M_I16M_P36H_V93I | 0.000 | 4.720 | 0.000 | 5.760 | 0.000 | 4.740 |
| 2899 | Y7M_I16M_P36H_V93I_R97A | 0.000 | 2.020 | 0.000 | 2.180 | 0.000 | 2.160 |
| 2900 | Y7M_I16M_P36H_V93I_R97F | 0.000 | 2.330 | 1.000 | −0.540 | 1.000 | −0.560 |
| 2901 | Y7M_I16M_P36S_R97A | 0.000 | 2.550 | 0.000 | 1.900 | 1.000 | 1.520 |
| 2902 | Y7M_I16M_P36S_V93I_R97A | 0.000 | 3.430 | 0.000 | 2.200 | 0.020 | 2.360 |
| 2903 | Y7M_I16M_P36S_V93I_R97F | 0.020 | 2.070 | 1.000 | 0.300 | 1.000 | 0.940 |
| 2904 | Y7M_I16M_P36S_V93R_R97A | 0.000 | 2.410 | 1.000 | −0.280 | 1.000 | −4.370 |
| 2905 | Y7M_I16M_P36W | 0.000 | 6.350 | 0.000 | 4.260 | 0.000 | 5.340 |
| 2906 | Y7M_I16M_P36W_P90F | 0.000 | 3.550 | 1.000 | 1.610 | 1.000 | 1.150 |
| 2907 | Y7M_I16M_P36W_P90F_V93I | 0.000 | 4.150 | 0.000 | 4.760 | 0.000 | 4.100 |
| 2908 | Y7M_I16M_P36W_P90I_V93I | 0.000 | 3.540 | 1.000 | 0.580 | 1.000 | 0.990 |
| 2909 | Y7M_I16M_P36W_P90W_R97A | 0.700 | 2.790 | 0.000 | 3.110 | 1.000 | 1.450 |
| 2910 | Y7M_I16M_P36W_P90W_V93I | 0.000 | 4.460 | 0.000 | 4.850 | 0.000 | 3.530 |
| 2911 | Y7M_I16M_P36W_P90W_V93I_R97A | 0.400 | 2.360 | 0.000 | 3.080 | 0.300 | 2.770 |
| 2912 | Y7M_I16M_P36W_P90W_V93I_R97F | 0.000 | 2.800 | 1.000 | 1.080 | 1.000 | 2.070 |
| 2913 | Y7M_I16M_P36W_P90Y | 0.000 | 2.380 | 1.000 | 1.260 | 0.960 | 1.770 |
| 2914 | Y7M_I16M_P36W_P90Y_R97A | 0.000 | 2.670 | 1.000 | 1.040 | 1.000 | 1.240 |
| 2915 | Y7M_I16M_P36W_P90Y_V93I | 0.000 | 5.490 | 0.000 | 2.660 | 0.480 | 2.460 |
| 2916 | Y7M_I16M_P36W_P90Y_V93I_R97A | 0.000 | 4.060 | 1.000 | 0.030 | 1.000 | −0.980 |
| 2917 | Y7M_I16M_P36W_P90Y_V93I_R97H | 0.010 | 2.220 | 0.510 | 1.410 | 1.000 | 1.550 |
| 2918 | Y7M_I16M_P36W_V93I | 0.000 | 8.310 | 0.000 | 5.200 | 0.000 | 5.540 |
| 2919 | Y7M_I16M_P36W_V93I_R97A | 0.000 | 3.690 | 1.000 | 3.280 | 0.010 | 1.550 |
| 2920 | Y7M_I16M_P36W_V93I_R97H | 0.000 | 4.190 | 0.000 | 3.500 | 0.040 | 3.490 |
| 2921 | Y7M_I16M_P36W_V93I_R97W | 0.030 | 3.300 | 0.000 | 2.910 | 0.350 | 2.680 |
| 2922 | Y7M_I16M_P36W_V93R_R97A | 0.020 | 2.620 | 0.000 | 3.590 | 1.000 | −0.200 |
| 2923 | Y7M_I16M_P90F | 0.000 | 6.190 | 0.000 | 5.450 | 0.000 | 6.550 |
| 2924 | Y7M_I16M_P90F_R97A | 0.000 | 3.780 | 0.060 | 2.260 | 1.000 | 1.550 |
| 2925 | Y7M_I16M_P90F_V93I | 0.000 | 6.700 | 0.000 | 6.240 | 0.000 | 6.330 |
| 2926 | Y7M_I16M_P90F_V93I_R97A | 0.000 | 4.100 | 0.010 | 3.260 | 1.000 | 1.380 |
| 2927 | Y7M_I16M_P90F_V93I_R97F | 0.000 | 3.040 | 0.000 | 3.920 | 1.000 | 1.910 |
| 2928 | Y7M_I16M_P90F_V93I_R97H | 0.000 | 4.230 | 0.000 | 2.900 | 1.000 | 1.370 |
| 2929 | Y7M_I16M_P90F_V93Q_R97A | 0.000 | 2.080 | 1.000 | 0.450 | 1.000 | 0.070 |
| 2930 | Y7M_I16M_P90I | 0.000 | 6.560 | 0.000 | 4.370 | 0.000 | 6.430 |
| 2931 | Y7M_I16M_P90I_R97F | 0.000 | 4.120 | 1.000 | 1.840 | 0.030 | 2.490 |
| 2932 | Y7M_I16M_P90I_V93I | 0.000 | 6.320 | 0.020 | 4.650 | 0.000 | 4.960 |
| 2933 | Y7M_I16M_P90I_V93I_R97A | 0.020 | 3.200 | 1.000 | 2.410 | 1.000 | 1.280 |
| 2934 | Y7M_I16M_P90I_V93K_R97W | 0.000 | 2.710 | 1.000 | 0.800 | 1.000 | 0.400 |
| 2935 | Y7M_I16M_P90I_V93R | 0.000 | 4.680 | 0.000 | 3.300 | 1.000 | 1.440 |
| 2936 | Y7M_I16M_P90S | 0.000 | 3.200 | 1.000 | −1.210 | 1.000 | −2.970 |
| 2937 | Y7M_I16M_P90W | 0.000 | 6.460 | 0.000 | 5.690 | 0.000 | 6.700 |
| 2938 | Y7M_I16M_P90W_R97H | 0.000 | 3.970 | 0.000 | 3.070 | 0.000 | 3.700 |
| 2939 | Y7M_I16M_P90W_V93I | 0.000 | 7.470 | 0.000 | 6.840 | 0.000 | 7.130 |
| 2940 | Y7M_I16M_P90W_V93I_R97A | 0.000 | 5.090 | 0.000 | 4.500 | 0.160 | 3.590 |
| 2941 | Y7M_I16M_P90W_V93I_R97H | 0.010 | 2.950 | 1.000 | 2.610 | 1.000 | 2.220 |
| 2942 | Y7M_I16M_P90W_V93I_R97W | 0.010 | 2.870 | 1.000 | 0.680 | 1.000 | −0.100 |
| 2943 | Y7M_I16M_P90Y | 0.000 | 5.710 | 0.000 | 3.240 | 0.000 | 4.340 |
| 2944 | Y7M_I16M_P90Y_R97A | 0.000 | 2.710 | 0.110 | 3.280 | 1.000 | 1.490 |
| 2945 | Y7M_I16M_P90Y_V93I | 0.000 | 6.250 | 0.000 | 4.870 | 0.000 | 5.130 |
| 2946 | Y7M_I16M_P90Y_V93I_R97A | 0.000 | 5.410 | 0.000 | 5.300 | 0.000 | 4.860 |
| 2947 | Y7M_I16M_P90Y_V93I_R97H | 0.000 | 4.000 | 0.000 | 3.550 | 1.000 | 1.380 |
| 2948 | Y7M_I16M_P90Y_V93Q_R97A | 0.000 | 2.890 | 1.000 | 0.650 | 1.000 | −0.300 |
| 2949 | Y7M_I16M_R97A | 0.000 | 4.360 | 0.000 | 3.520 | 0.230 | 2.980 |
| 2950 | Y7M_I16M_R97F | 0.000 | 3.210 | 1.000 | 1.230 | 1.000 | 2.200 |
| 2951 | Y7M_I16M_R97H | 0.000 | 3.740 | 0.010 | 2.220 | 1.000 | 1.700 |
| 2952 | Y7M_I16M_R97W | 1.000 | 2.490 | 0.000 | 4.210 | 0.000 | 3.440 |
| 2953 | Y7M_I16M_V93I | 0.000 | 4.010 | 0.570 | 1.280 | 0.030 | 1.510 |
| 2954 | Y7M_I16M_V93I_R97A | 0.010 | 3.760 | 0.130 | 2.410 | 0.000 | 2.580 |
| 2955 | Y7M_I16M_V93I_R97F | 0.000 | 4.770 | 0.000 | 3.880 | 0.000 | 3.220 |
| 2956 | Y7M_I16M_V93I_R97H | 0.000 | 3.680 | 0.000 | 4.220 | 0.050 | 3.590 |
| 2957 | Y7M_I16M_V93I_R97W | 0.000 | 4.660 | 0.000 | 3.730 | 1.000 | 3.070 |
| 2958 | Y7M_I16M_V93K | 0.000 | 4.310 | 0.000 | 3.890 | 0.000 | 3.400 |
| 2959 | Y7M_I16M_V93K_R97A | 1.000 | 0.910 | 0.000 | 2.750 | 1.000 | 0.010 |
| 2960 | Y7M_I16M_V93K_R97H | 1.000 | 1.340 | 0.040 | 2.330 | 1.000 | 0.470 |
| 2961 | Y7M_I16M_V93K_R97W | 1.000 | 2.310 | 0.000 | 3.610 | 1.000 | 0.020 |
| 2962 | Y7M_I16M_V93Q_R97F | 0.000 | 3.860 | 0.000 | 3.210 | 1.000 | −0.220 |
| 2963 | Y7M_I16M_V93R | 0.000 | 6.680 | 0.000 | 6.930 | 0.000 | 4.310 |
| 2964 | Y7M_I16M_V93R_R97A | 0.000 | 5.680 | 0.010 | 4.940 | 0.490 | 3.230 |
| 2965 | Y7M_I16M_V93R_R97A_R119M | 0.000 | 2.830 | 1.000 | −0.210 | 1.000 | −3.830 |
| 2966 | Y7M_I16M_V93R_R97A_R119Q | 0.000 | 3.710 | 1.000 | 1.390 | 1.000 | 0.090 |
| 2967 | Y7M_I16M_V93R_R97P | 0.000 | 3.130 | 1.000 | −1.200 | 1.000 | −1.900 |
| 2968 | Y7M_I16M_V93R_R97W | 0.000 | 2.420 | 1.000 | −0.800 | 1.000 | −1.880 |
| 2969 | Y7M_I16W | 0.000 | 9.740 | 0.000 | 6.090 | 0.000 | 8.330 |
| 2970 | Y7M_I16W_P36A | 0.000 | 5.090 | 0.000 | 3.430 | 0.000 | 4.900 |
| 2971 | Y7M_I16W_P36A_V93I | 0.000 | 6.150 | 0.010 | 3.380 | 0.000 | 5.520 |
| 2972 | Y7M_I16W_P36A_V93I_R97A | 0.000 | 4.130 | 0.000 | 3.740 | 0.000 | 4.870 |
| 2973 | Y7M_I16W_P36H | 0.000 | 3.380 | 0.000 | 3.260 | 1.000 | 1.590 |
| 2974 | Y7M_I16W_P36H_V93I | 0.000 | 4.270 | 0.230 | 2.740 | 0.200 | 2.700 |
| 2975 | Y7M_I16W_P36H_V93I_R97A | 0.000 | 4.070 | 0.190 | 3.620 | 1.000 | 0.510 |
| 2976 | Y7M_I16W_P36S_V93I | 0.000 | 4.810 | 0.000 | 3.830 | 0.000 | 4.750 |
| 2977 | Y7M_I16W_P36S_V93I_R97A | 1.000 | 0.780 | 0.000 | 2.680 | 1.000 | 2.680 |
| 2978 | Y7M_I16W_P90F_V93I | 0.040 | 3.790 | 0.000 | 4.510 | 0.000 | 3.480 |
| 2979 | Y7M_I16W_P90F_V93I_R97A | 0.000 | 2.320 | 0.520 | 1.660 | 1.000 | 0.420 |
| 2980 | Y7M_I16W_P90I | 0.000 | 4.620 | 0.000 | 2.970 | 0.000 | 3.650 |
| 2981 | Y7M_I16W_P90I_V93I | 0.000 | 5.070 | 0.000 | 4.550 | 0.000 | 4.550 |
| 2982 | Y7M_I16W_P90I_V93I_R97A | 1.000 | 3.480 | 0.010 | 3.740 | 0.870 | 2.780 |
| 2983 | Y7M_I16W_P90W | 0.000 | 4.380 | 1.000 | 1.210 | 1.000 | 1.390 |
| 2984 | Y7M_I16W_P90W_V93I | 0.000 | 5.100 | 0.010 | 3.510 | 0.000 | 3.100 |
| 2985 | Y7M_I16W_P90W_V93I_R97F | 0.000 | 5.940 | 0.000 | 4.800 | 0.000 | 4.980 |
| 2986 | Y7M_I16W_P90Y | 0.000 | 3.420 | 1.000 | 1.600 | 1.000 | 1.060 |
| 2987 | Y7M_I16W_P90Y_R97A | 0.000 | 2.170 | 0.000 | 1.620 | 0.840 | 1.170 |
| 2988 | Y7M_I16W_P90Y_V93I | 0.000 | 4.180 | 0.000 | 4.100 | 0.000 | 3.240 |
| 2989 | Y7M_I16W_R97A | 0.000 | 7.350 | 0.000 | 7.360 | 0.000 | 5.560 |
| 2990 | Y7M_I16W_R97F | 0.000 | 5.440 | 0.000 | 4.020 | 0.000 | 3.460 |
| 2991 | Y7M_I16W_R97H | 0.000 | 6.310 | 0.000 | 6.000 | 0.000 | 5.160 |
| 2992 | Y7M_I16W_R97W | 0.000 | 6.160 | 0.000 | 3.650 | 0.910 | 2.440 |
| 2993 | Y7M_I16W_V93I | 0.000 | 11.090 | 0.000 | 5.150 | 0.000 | 6.770 |
| 2994 | Y7M_I16W_V93I_R97A | 0.000 | 7.420 | 0.000 | 6.640 | 0.000 | 6.860 |
| 2995 | Y7M_I16W_V93I_R97F | 0.000 | 5.960 | 0.000 | 5.730 | 0.000 | 5.230 |
| 2996 | Y7M_I16W_V93I_R97H | 0.000 | 6.100 | 0.000 | 6.120 | 0.000 | 5.880 |
| 2997 | Y7M_I16W_V93I_R97W | 0.000 | 6.130 | 0.000 | 5.330 | 0.000 | 5.540 |
| 2998 | Y7M_I16W_V93Q | 0.000 | 3.330 | 0.470 | 2.370 | 0.000 | 1.620 |
| 2999 | Y7M_I16W_V93Q_R97F | 0.000 | 2.690 | 1.000 | 0.610 | 1.000 | 1.120 |
| 3000 | Y7M_I16W_V93R | 0.000 | 2.210 | 0.020 | 4.470 | 1.000 | 0.900 |
| 3001 | Y7M_I16W_V93R_R97F | 0.000 | 3.220 | 1.000 | 1.320 | 1.000 | −0.930 |
| 3002 | Y7M_P36A | 0.000 | 5.300 | 1.000 | 0.570 | 1.000 | 1.420 |
| 3003 | Y7M_P36A_P90F | 0.000 | 5.380 | 0.000 | 5.130 | 0.000 | 3.990 |
| 3004 | Y7M_P36A_P90F_V93I | 0.000 | 7.060 | 0.000 | 6.050 | 0.000 | 6.220 |
| 3005 | Y7M_P36A_P90F_V93I_R97H | 0.000 | 3.290 | 0.010 | 2.550 | 1.000 | 0.640 |
| 3006 | Y7M_P36A_P90F_V93Q | 0.020 | 3.790 | 1.000 | −0.450 | 1.000 | −1.630 |
| 3007 | Y7M_P36A_P90I | 0.010 | 3.150 | 1.000 | 1.300 | 1.000 | 1.780 |
| 3008 | Y7M_P36A_P90I_R97A | 1.000 | −1.220 | 0.020 | 2.250 | 1.000 | −1.170 |
| 3009 | Y7M_P36A_P90I_V93I | 0.000 | 4.200 | 0.780 | 2.270 | 0.150 | 3.170 |
| 3010 | Y7M_P36A_P90I_V93I_R97A | 0.020 | 2.520 | 1.000 | 1.180 | 1.000 | −1.040 |
| 3011 | Y7M_P36A_P90S | 0.000 | 4.260 | 1.000 | −3.170 | 1.000 | −2.450 |
| 3012 | Y7M_P36A_P90W | 0.000 | 6.060 | 0.000 | 4.940 | 0.010 | 3.630 |
| 3013 | Y7M_P36A_P90W_R97W | 1.000 | 0.090 | 0.050 | 2.240 | 1.000 | 1.340 |
| 3014 | Y7M_P36A_P90W_V93I | 0.000 | 6.760 | 0.000 | 6.610 | 0.000 | 5.990 |
| 3015 | Y7M_P36A_P90W_V93I_R97A | 0.410 | 2.660 | 0.000 | 2.610 | 1.000 | 0.800 |
| 3016 | Y7M_P36A_P90Y_R97A | 0.760 | 2.260 | 0.000 | 2.220 | 1.000 | 0.950 |
| 3017 | Y7M_P36A_P90Y_V93I | 0.000 | 6.970 | 0.000 | 5.560 | 0.000 | 4.690 |
| 3018 | Y7M_P36A_P90Y_V93I_R97W | 1.000 | 1.300 | 0.010 | 2.250 | 1.000 | 1.520 |
| 3019 | Y7M_P36A_R97A | 0.000 | 4.550 | 0.110 | 2.540 | 1.000 | 1.840 |
| 3020 | Y7M_P36A_R97F | 0.000 | 4.610 | 0.000 | 3.670 | 1.000 | 2.350 |
| 3021 | Y7M_P36A_R97H | 0.710 | 2.740 | 0.000 | 3.290 | 1.000 | 0.810 |
| 3022 | Y7M_P36A_V93I | 0.000 | 4.970 | 1.000 | 0.610 | 1.000 | 0.340 |
| 3023 | Y7M_P36A_V93I_R97H | 1.000 | 1.970 | 0.000 | 3.370 | 1.000 | 2.520 |
| 3024 | Y7M_P36A_V93K | 0.000 | 3.940 | 0.000 | 4.150 | 1.000 | 1.260 |
| 3025 | Y7M_P36A_V93K_R97W | 0.000 | 2.920 | 1.000 | 0.110 | 1.000 | −2.250 |
| 3026 | Y7M_P36A_V93Q | 0.000 | 5.140 | 0.000 | 4.090 | 1.000 | 2.120 |
| 3027 | Y7M_P36A_V93R | 0.000 | 5.370 | 0.250 | 3.390 | 1.000 | 2.010 |
| 3028 | Y7M_P36A_V93R_R97W | 0.000 | 3.680 | 0.000 | 2.560 | 1.000 | −1.420 |
| 3029 | Y7M_P36H | 0.000 | 6.000 | 0.000 | 6.490 | 0.500 | 2.890 |
| 3030 | Y7M_P36H_P90F | 0.000 | 5.260 | 0.000 | 4.710 | 1.000 | 1.460 |
| 3031 | Y7M_P36H_P90F_V93I | 0.000 | 4.030 | 0.010 | 4.240 | 0.690 | 0.970 |
| 3032 | Y7M_P36H_P90I | 0.000 | 2.970 | 1.000 | 1.140 | 1.000 | 1.500 |
| 3033 | Y7M_P36H_P90I_V93I | 0.000 | 3.980 | 0.200 | 3.450 | 1.000 | 1.960 |
| 3034 | Y7M_P36H_P90W | 0.080 | 2.840 | 0.010 | 3.120 | 1.000 | 1.960 |
| 3035 | Y7M_P36H_P90W_V93I | 0.000 | 4.920 | 0.000 | 5.820 | 0.000 | 3.560 |
| 3036 | Y7M_P36H_P90Y | 0.000 | 5.020 | 0.090 | 3.090 | 1.000 | 1.540 |
| 3037 | Y7M_P36H_P90Y_R97W | 0.000 | 2.700 | 1.000 | −3.830 | 1.000 | −1.940 |
| 3038 | Y7M_P36H_P90Y_V93I | 0.000 | 4.740 | 0.510 | 2.880 | 1.000 | 0.890 |
| 3039 | Y7M_P36H_R97F | 0.350 | 2.360 | 0.000 | 2.830 | 1.000 | −2.220 |
| 3040 | Y7M_P36H_V93I | 0.000 | 6.480 | 0.000 | 6.470 | 0.060 | 3.810 |
| 3041 | Y7M_P36H_V93I_R97H | 0.000 | 3.100 | 0.000 | 3.230 | 1.000 | 1.580 |
| 3042 | Y7M_P36H_V93I_R97W | 0.000 | 2.490 | 1.000 | 0.620 | 1.000 | −0.050 |
| 3043 | Y7M_P36H_V93R_R97A | 0.000 | 3.520 | 1.000 | 2.010 | 1.000 | 1.460 |
| 3044 | Y7M_P36R | 0.000 | 5.350 | 0.000 | 4.850 | 1.000 | 1.650 |
| 3045 | Y7M_P36R_R97A | 0.000 | 5.280 | 1.000 | 0.130 | 1.000 | −0.200 |
| 3046 | Y7M_P36R_V93I_R97A | 1.000 | −1.310 | 1.000 | −0.460 | 0.000 | 3.040 |
| 3047 | Y7M_P36S | 0.000 | 5.900 | 0.000 | 5.360 | 0.000 | 5.230 |
| 3048 | Y7M_P36S_P90F | 0.000 | 3.710 | 1.000 | 1.570 | 0.000 | 3.370 |
| 3049 | Y7M_P36S_P90F_V93I | 0.000 | 3.820 | 0.000 | 5.760 | 0.000 | 4.210 |
| 3050 | Y7M_P36S_P90I_V93I | 0.000 | 4.140 | 1.000 | 0.170 | 1.000 | 2.270 |
| 3051 | Y7M_P36S_P90I_V93I_R97H | 0.000 | 2.200 | 1.000 | −1.350 | 1.000 | −1.050 |
| 3052 | Y7M_P36S_P90W | 0.000 | 3.530 | 0.530 | 2.510 | 0.190 | 2.700 |
| 3053 | Y7M_P36S_P90W_V93I | 0.000 | 4.410 | 0.000 | 4.880 | 0.000 | 3.710 |
| 3054 | Y7M_P36S_P90Y_R97A | 1.000 | 1.610 | 0.000 | 2.070 | 1.000 | −1.240 |
| 3055 | Y7M_P36S_P90Y_V93I | 0.000 | 2.940 | 0.000 | 4.280 | 0.000 | 3.600 |
| 3056 | Y7M_P36S_R97A | 0.000 | 2.870 | 0.060 | 2.560 | 1.000 | 1.420 |
| 3057 | Y7M_P36S_R97F | 0.000 | 3.980 | 0.000 | 2.510 | 1.000 | 2.240 |
| 3058 | Y7M_P36S_R97H | 1.000 | −0.460 | 0.020 | 2.770 | 1.000 | −1.610 |
| 3059 | Y7M_P36S_R97W | 0.000 | 2.080 | 1.000 | 1.250 | 0.000 | 1.790 |
| 3060 | Y7M_P36S_V93I | 0.000 | 5.490 | 1.000 | 1.420 | 1.000 | 0.910 |
| 3061 | Y7M_P36S_V93I_R97A | 0.000 | 3.310 | 0.830 | 3.380 | 1.000 | 0.730 |
| 3062 | Y7M_P36S_V93I_R97F | 0.000 | 2.600 | 0.000 | 2.720 | 1.000 | 0.410 |
| 3063 | Y7M_P36S_V93I_R97H | 0.000 | 3.110 | 0.160 | 2.130 | 1.000 | −0.490 |
| 3064 | Y7M_P36S_V93I_R97W | 0.000 | 3.350 | 0.000 | 3.010 | 0.000 | 3.480 |
| 3065 | Y7M_P36S_V93K | 1.000 | 1.090 | 0.010 | 2.410 | 1.000 | −2.470 |
| 3066 | Y7M_P36S_V93Q | 0.030 | 3.810 | 0.000 | 3.540 | 1.000 | 1.640 |
| 3067 | Y7M_P36S_V93Q_R97A | 0.000 | 3.350 | 0.000 | 3.570 | 0.000 | 3.100 |
| 3068 | Y7M_P36S_V93R_R97A | 0.000 | 3.770 | 0.000 | 3.140 | 1.000 | −1.380 |
| 3069 | Y7M_P36S_V93R_R97F | 1.000 | 2.110 | 1.000 | 1.780 | 0.000 | 2.970 |
| 3070 | Y7M_P36W | 0.000 | 6.250 | 0.000 | 2.930 | 0.000 | 4.220 |
| 3071 | Y7M_P36W_P90F | 0.000 | 4.090 | 0.000 | 5.330 | 0.000 | 3.730 |
| 3072 | Y7M_P36W_P90F_R97A | 0.000 | 3.680 | 0.010 | 2.840 | 1.000 | −0.010 |
| 3073 | Y7M_P36W_P90F_R97H | 0.000 | 2.090 | 1.000 | 1.260 | 0.170 | 2.310 |
| 3074 | Y7M_P36W_P90F_V93I | 0.000 | 6.430 | 0.000 | 6.870 | 0.000 | 5.410 |
| 3075 | Y7M_P36W_P90F_V93I_R97A | 0.000 | 3.680 | 0.000 | 3.210 | 1.000 | 2.630 |
| 3076 | Y7M_P36W_P90F_V93I_R97F | 0.000 | 3.860 | 0.010 | 2.720 | 0.070 | 2.480 |
| 3077 | Y7M_P36W_P90F_V93I_R97H | 1.000 | 1.840 | 0.030 | 2.360 | 1.000 | −0.090 |
| 3078 | Y7M_P36W_P90F_V93I_R97W | 0.000 | 3.370 | 0.700 | 1.880 | 0.150 | 2.770 |
| 3079 | Y7M_P36W_P90I | 0.020 | 3.270 | 0.000 | 3.030 | 0.000 | 4.000 |
| 3080 | Y7M_P36W_P90I_V93I | 0.000 | 6.290 | 0.000 | 4.460 | 0.210 | 3.080 |
| 3081 | Y7M_P36W_P90I_V93I_R97A | 0.000 | 2.520 | 0.000 | 2.790 | 0.000 | 2.110 |
| 3082 | Y7M_P36W_P90I_V93R | 0.000 | 3.400 | 1.000 | 1.760 | 1.000 | −2.290 |
| 3083 | Y7M_P36W_P90S | 0.000 | 2.400 | 1.000 | 1.010 | 1.000 | 2.320 |
| 3084 | Y7M_P36W_P90S_V93I_R97A | 0.000 | 2.400 | 1.000 | 1.000 | 0.000 | 3.700 |
| 3085 | Y7M_P36W_P90W | 0.000 | 6.460 | 0.000 | 6.710 | 0.000 | 4.920 |
| 3086 | Y7M_P36W_P90W_R97A | 0.000 | 4.540 | 0.000 | 3.070 | 1.000 | 1.180 |
| 3087 | Y7M_P36W_P90W_V93I | 0.000 | 8.010 | 0.000 | 7.470 | 0.000 | 5.980 |
| 3088 | Y7M_P36W_P90W_V93I_R97A | 0.000 | 4.630 | 0.000 | 4.710 | 1.000 | 1.740 |
| 3089 | Y7M_P36W_P90W_V93I_R97F | 0.000 | 4.070 | 0.000 | 4.850 | 0.570 | 2.380 |
| 3090 | Y7M_P36W_P90W_V93I_R97H | 0.000 | 4.650 | 0.000 | 4.220 | 1.000 | 2.520 |
| 3091 | Y7M_P36W_P90Y | 0.000 | 5.320 | 0.000 | 4.340 | 0.000 | 4.000 |
| 3092 | Y7M_P36W_P90Y_V93I | 0.000 | 7.170 | 0.000 | 6.960 | 0.000 | 5.620 |
| 3093 | Y7M_P36W_P90Y_V93I_R97A | 0.000 | 3.950 | 0.000 | 4.050 | 0.490 | 1.880 |
| 3094 | Y7M_P36W_P90Y_V93I_R97H | 0.000 | 4.430 | 0.000 | 3.200 | 1.000 | 2.590 |
| 3095 | Y7M_P36W_P90Y_V93I_R97W | 0.000 | 2.970 | 1.000 | 1.150 | 1.000 | −1.610 |
| 3096 | Y7M_P36W_P90Y_V93R | 0.000 | 3.060 | 0.040 | 1.220 | 1.000 | −1.740 |
| 3097 | Y7M_P36W_R97A | 0.000 | 4.420 | 0.000 | 3.370 | 0.000 | 4.510 |
| 3098 | Y7M_P36W_R97F | 0.000 | 3.580 | 1.000 | 1.270 | 1.000 | 0.620 |
| 3099 | Y7M_P36W_R97H | 0.000 | 4.620 | 0.000 | 4.190 | 0.010 | 3.730 |
| 3100 | Y7M_P36W_V93I | 0.000 | 7.670 | 0.000 | 3.480 | 0.000 | 4.190 |
| 3101 | Y7M_P36W_V93I_R97A | 0.000 | 5.480 | 0.000 | 2.980 | 0.040 | 3.080 |
| 3102 | Y7M_P36W_V93I_R97F | 0.000 | 4.040 | 0.000 | 3.740 | 0.000 | 3.990 |
| 3103 | Y7M_P36W_V93I_R97H | 0.000 | 4.870 | 0.000 | 4.210 | 0.000 | 4.300 |
| 3104 | Y7M_P36W_V93I_R97W | 0.000 | 3.410 | 0.080 | 3.660 | 0.130 | 2.900 |
| 3105 | Y7M_P36W_V93K | 0.000 | 2.440 | 1.000 | 1.160 | 0.010 | 2.350 |
| 3106 | Y7M_P36W_V93Q | 0.010 | 2.860 | 0.010 | 3.290 | 0.000 | 3.230 |
| 3107 | Y7M_P36W_V93R | 0.000 | 4.130 | 0.000 | 4.160 | 0.110 | 2.790 |
| 3108 | Y7M_P36W_V93R_R97A | 0.000 | 5.150 | 0.000 | 5.960 | 0.000 | 4.130 |
| 3109 | Y7M_P36W_V93R_R97F | 0.000 | 2.270 | 1.000 | 1.150 | 0.000 | 2.320 |
| 3110 | Y7M_P90F | 0.000 | 5.980 | 0.000 | 4.630 | 0.000 | 6.140 |
| 3111 | Y7M_P90F_R97A | 0.000 | 4.060 | 0.000 | 3.990 | 1.000 | 1.210 |
| 3112 | Y7M_P90F_R97F | 1.000 | 0.930 | 0.010 | 2.460 | 1.000 | 0.510 |
| 3113 | Y7M_P90F_R97H | 0.000 | 4.070 | 0.010 | 2.940 | 1.000 | 2.340 |
| 3114 | Y7M_P90F_V93I | 0.000 | 7.020 | 0.000 | 6.310 | 0.000 | 6.100 |
| 3115 | Y7M_P90F_V93I_R97H | 0.000 | 4.090 | 0.000 | 3.860 | 1.000 | 1.460 |
| 3116 | Y7M_P90F_V93I_R97W | 0.000 | 3.460 | 1.000 | 2.000 | 1.000 | −0.310 |
| 3117 | Y7M_P90F_V93Q | 0.000 | 3.630 | 0.590 | 3.430 | 1.000 | 2.050 |
| 3118 | Y7M_P90F_V93R | 0.000 | 3.580 | 0.000 | 4.020 | 1.000 | −0.670 |
| 3119 | Y7M_P90F_V93R_R97A | 0.000 | 4.140 | 0.000 | 3.620 | 0.000 | 3.250 |
| 3120 | Y7M_P90I | 0.000 | 4.240 | 1.000 | 1.690 | 0.000 | 3.450 |
| 3121 | Y7M_P90I_R97A | 1.000 | 2.880 | 0.000 | 3.450 | 1.000 | 1.260 |
| 3122 | Y7M_P90I_R97F | 0.340 | 1.640 | 0.000 | 2.680 | 1.000 | 0.990 |
| 3123 | Y7M_P90I_V93I | 0.000 | 7.780 | 0.000 | 4.690 | 0.000 | 5.380 |
| 3124 | Y7M_P90I_V93I_R97A | 1.000 | 2.640 | 0.000 | 3.330 | 1.000 | 1.070 |
| 3125 | Y7M_P90I_V93I_R97H | 0.000 | 3.890 | 0.000 | 3.440 | 1.000 | 1.780 |
| 3126 | Y7M_P90I_V93I_R97W | 0.000 | 3.480 | 0.000 | 3.850 | 0.330 | 2.360 |
| 3127 | Y7M_P90I_V93K | 1.000 | 2.120 | 0.000 | 4.190 | 1.000 | −1.150 |
| 3128 | Y7M_P90I_V93Q | 0.000 | 5.170 | 0.260 | 2.960 | 1.000 | 0.940 |
| 3129 | Y7M_P90I_V93R_R97A | 0.010 | 3.730 | 0.690 | 2.840 | 1.000 | −0.980 |
| 3130 | Y7M_P90I_V93R_R97W | 0.020 | 2.240 | 1.000 | 1.100 | 0.070 | 1.600 |
| 3131 | Y7M_P90S_R97H | 0.000 | 3.200 | 0.000 | 2.880 | 1.000 | 0.420 |
| 3132 | Y7M_P90S_V93I | 0.000 | 4.310 | 1.000 | −1.380 | 1.000 | −1.380 |
| 3133 | Y7M_P90S_V93I_R97F | 1.000 | 1.980 | 0.000 | 2.630 | 0.420 | 3.010 |
| 3134 | Y7M_P90S_V93I_R97H | 0.000 | 4.390 | 0.000 | 3.780 | 1.000 | 1.530 |
| 3135 | Y7M_P90S_V93I_R97W | 0.170 | 2.390 | 0.000 | 3.300 | 1.000 | 2.400 |
| 3136 | Y7M_P90S_V93Q | 0.010 | 1.950 | 0.320 | 2.200 | 0.000 | 2.660 |
| 3137 | Y7M_P90S_V93Q_R97A | 0.270 | 2.310 | 1.000 | 1.820 | 0.010 | 2.260 |
| 3138 | Y7M_P90W | 0.000 | 6.440 | 0.000 | 6.500 | 0.000 | 6.190 |
| 3139 | Y7M_P90W_R97A | 0.000 | 4.080 | 0.070 | 3.260 | 1.000 | 2.200 |
| 3140 | Y7M_P90W_R97F | 0.000 | 4.140 | 0.000 | 3.750 | 0.000 | 3.540 |
| 3141 | Y7M_P90W_R97H | 0.000 | 4.450 | 0.020 | 2.990 | 1.000 | 2.080 |
| 3142 | Y7M_P90W_R97W | 0.040 | 3.770 | 0.000 | 4.380 | 1.000 | −0.370 |
| 3143 | Y7M_P90W_V93I | 0.000 | 6.170 | 0.000 | 5.440 | 0.000 | 6.250 |
| 3144 | Y7M_P90W_V93I_R97A | 0.000 | 4.130 | 0.000 | 5.150 | 1.000 | 2.330 |
| 3145 | Y7M_P90W_V93I_R97F | 0.000 | 2.340 | 0.010 | 4.080 | 0.180 | 1.050 |
| 3146 | Y7M_P90W_V93I_R97H | 0.000 | 4.920 | 0.000 | 4.520 | 0.870 | 2.670 |
| 3147 | Y7M_P90W_V93Q | 0.000 | 5.100 | 0.000 | 4.780 | 1.000 | 2.110 |
| 3148 | Y7M_P90W_V93Q_R97W | 0.020 | 2.250 | 1.000 | −0.500 | 1.000 | −2.130 |
| 3149 | Y7M_P90W_V93R | 0.000 | 6.600 | 0.190 | 3.910 | 1.000 | 2.380 |
| 3150 | Y7M_P90W_V93R_R97A_R119Q | 0.000 | 2.070 | 1.000 | 0.560 | 1.000 | 0.360 |
| 3151 | Y7M_P90W_V93R_R97F | 1.000 | 1.810 | 0.000 | 2.850 | 1.000 | −0.390 |
| 3152 | Y7M_P90W_V93R_R97H | 0.000 | 3.050 | 0.010 | 3.090 | 1.000 | −0.060 |
| 3153 | Y7M_P90Y | 0.000 | 6.840 | 0.000 | 5.700 | 0.000 | 6.380 |
| 3154 | Y7M_P90Y_R97F | 0.000 | 3.210 | 0.000 | 2.230 | 1.000 | −0.310 |
| 3155 | Y7M_P90Y_R97H | 0.110 | 2.480 | 0.000 | 3.150 | 0.090 | 3.030 |
| 3156 | Y7M_P90Y_V93I | 0.000 | 6.440 | 0.000 | 6.900 | 0.000 | 4.480 |
| 3157 | Y7M_P90Y_V93I_R97F | 1.000 | 2.710 | 0.000 | 2.980 | 1.000 | 1.260 |
| 3158 | Y7M_P90Y_V93Q_R97A | 1.000 | −1.520 | 0.010 | 2.640 | 1.000 | −1.810 |
| 3159 | Y7M_P90Y_V93R | 0.000 | 4.580 | 0.000 | 3.500 | 0.000 | 3.040 |
| 3160 | Y7M_R97A | 0.000 | 4.870 | 0.010 | 3.010 | 0.030 | 3.590 |
| 3161 | Y7M_R97F | 0.000 | 3.290 | 1.000 | 1.970 | 1.000 | 2.180 |
| 3162 | Y7M_R97H | 0.000 | 3.920 | 0.000 | 3.590 | 1.000 | 2.660 |
| 3163 | Y7M_R97W | 0.000 | 4.000 | 0.000 | 4.250 | 0.000 | 4.420 |
| 3164 | Y7M_V93I | 0.000 | 4.010 | 0.090 | 1.690 | 0.140 | 1.370 |
| 3165 | Y7M_V93I_R97A | 0.000 | 3.090 | 0.030 | 2.860 | 1.000 | 2.320 |
| 3166 | Y7M_V93I_R97F | 0.000 | 3.810 | 0.000 | 3.790 | 1.000 | 3.310 |
| 3167 | Y7M_V93I_R97H | 0.040 | 2.530 | 0.000 | 3.930 | 1.000 | 1.810 |
| 3168 | Y7M_V93I_R97W | 0.000 | 3.160 | 0.030 | 2.650 | 0.110 | 2.930 |
| 3169 | Y7M_V93K | 0.000 | 3.990 | 0.000 | 5.400 | 0.150 | 3.100 |
| 3170 | Y7M_V93K_R97A | 0.000 | 3.360 | 0.230 | 2.980 | 0.000 | 2.920 |
| 3171 | Y7M_V93K_R97F | 0.000 | 2.980 | 1.000 | 1.760 | 1.000 | −0.510 |
| 3172 | Y7M_V93K_R97H | 0.000 | 2.580 | 0.200 | 2.350 | 1.000 | 0.930 |
| 3173 | Y7M_V93K_R97W | 0.000 | 3.310 | 1.000 | 1.260 | 1.000 | −1.940 |
| 3174 | Y7M_V93Q | 0.000 | 5.210 | 0.010 | 3.320 | 0.340 | 3.160 |
| 3175 | Y7M_V93Q_R97H | 0.040 | 3.220 | 0.010 | 2.940 | 1.000 | −1.760 |
| 3176 | Y7M_V93R | 0.000 | 5.520 | 0.000 | 6.510 | 0.000 | 3.040 |
| 3177 | Y7M_V93R_R97A | 0.000 | 4.210 | 0.000 | 4.750 | 1.000 | 0.600 |
| 3178 | Y7M_V93R_R97F | 0.000 | 5.190 | 0.000 | 3.380 | 1.000 | 0.940 |
| 3179 | Y7P_I16F_P90F_V93I | 0.000 | 2.640 | 0.000 | 1.090 | 0.000 | 1.780 |
| 3180 | Y7P_I16F_P90Y_R97A | 1.000 | 1.870 | 0.030 | 2.270 | 1.000 | 0.210 |
| 3181 | Y7P_I16L_P36A_V93I_R97F | 0.010 | 2.060 | 1.000 | −0.220 | 1.000 | −0.370 |
| 3182 | Y7P_I16L_V93I_R97W | 0.000 | 2.440 | 1.000 | 1.920 | 1.000 | 0.970 |
| 3183 | Y7P_I16M_P90W_V93I | 0.000 | 3.680 | 0.000 | 2.940 | 0.070 | 3.050 |
| 3184 | Y7P_I16M_P90W_V93I_R97A | 1.000 | 1.270 | 0.030 | 2.220 | 1.000 | 0.060 |
| 3185 | Y7P_I16M_P90Y_V93I | 0.000 | 2.960 | 1.000 | 0.560 | 1.000 | 2.120 |
| 3186 | Y7P_I16M_V93Q | 0.000 | 4.450 | 0.000 | 4.490 | 0.000 | 3.370 |
| 3187 | Y7P_P36A_P90W_V93I | 0.000 | 2.360 | 0.980 | 1.340 | 1.000 | 0.920 |
| 3188 | Y7P_P90F | 0.000 | 4.550 | 0.000 | 3.640 | 0.000 | 3.580 |
| 3189 | Y7P_P90W_V93I | 0.000 | 3.600 | 0.000 | 2.700 | 0.000 | 3.820 |
| 3190 | Y7P_P90W_V93I_R97A | 0.020 | 2.610 | 1.000 | −0.070 | 1.000 | −1.380 |
| 3191 | Y7P_P90W_V93Q | 0.000 | 2.550 | 1.000 | 0.890 | 1.000 | 0.920 |
| 3192 | Y7P_P90Y_V93I | 0.000 | 2.160 | 1.000 | −0.880 | 1.000 | 0.270 |

*adjusted p-val under dU/dI primer tolerance condition
**fitness_combined score under dU/dI primer tolerance condition
^{†}adjusted p-val under dU primer tolerance condition
^{††}fitness_combined score under dU primer tolerance condition
^{‡}adjusted p-val under dUTP incorporation tolerance condition
^{‡‡}fitness_combined score under dUTP incorporation tolerance condition

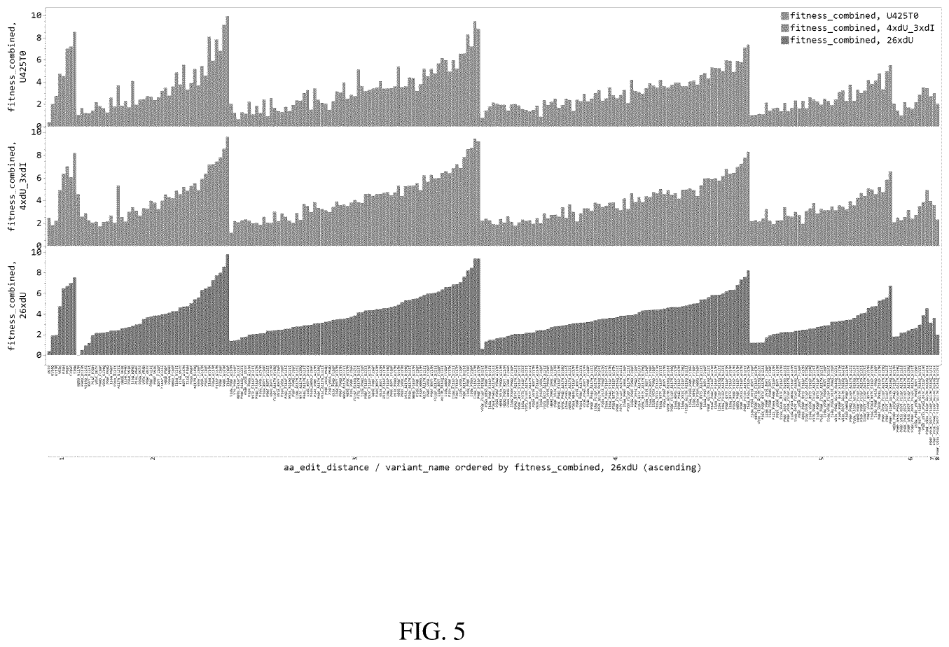

From the 15p2s library, 245 combinatorial substitution variants were identified as having improved dU/dI primer tolerance, dU primer tolerance, or dUTP incorporation tolerance. The list of variants with improved dU/dI primer tolerance, dU primer tolerance, or dUTP incorporation tolerance is contained in Table 5 below. A summary of this data is shown in FIG. 5.

TABLE 5
15p2s Library variants with improved dU/dI primer tolerance, dU primer tolerance, or dUTP incorporation tolerance.

| SEQ | | p-val | | p-val | | p-val | |
|---|---|---|---|---|---|---|---|
| ID NO | Substitutions | dI/dU* | dI/dU** | dU^{†} | dU^{††} | dUTP^{‡} | dUTP^{‡‡} |
| 242 | A117M | 0.000 | 2.220 | 0.000 | 1.960 | 0.000 | 2.760 |
| 3193 | A117M_D123I | 0.000 | 5.330 | 0.000 | 2.410 | 0.000 | 3.720 |
| 3194 | A117M_R119Q | 0.000 | 5.540 | 0.000 | 5.430 | 0.000 | 5.200 |
| 3195 | A117M_R119Q_D123I | 0.000 | 6.440 | 0.000 | 6.380 | 0.000 | 6.180 |
| 235 | F116P | 0.000 | 6.060 | 0.000 | 7.000 | 0.000 | 7.210 |
| 3196 | F116P_A117M | 0.000 | 7.450 | 0.000 | 7.750 | 0.000 | 7.860 |
| 3197 | F116P_A117M_D123I | 0.000 | 5.960 | 0.000 | 6.070 | 0.000 | 4.790 |
| 3198 | F116P_A117M_R119Q | 0.000 | 3.040 | 0.080 | 2.400 | 0.000 | 1.700 |
| 3199 | F116P_A117M_R119Q_D123I | 0.000 | 4.990 | 0.000 | 4.830 | 0.000 | 3.620 |
| 3200 | F116P_D123I | 0.000 | 3.980 | 0.000 | 3.980 | 0.000 | 3.200 |
| 3201 | F116P_R119Q_D123I | 0.000 | 4.060 | 0.000 | 3.840 | 0.000 | 2.730 |
| 3202 | H89D_A117M | 0.000 | 4.590 | 1.000 | −0.040 | 0.010 | 1.050 |
| 3203 | H89D_A117M_D123I | 0.000 | 3.670 | 0.000 | 3.530 | 0.000 | 3.990 |
| 3204 | H89D_F116P | 0.000 | 4.190 | 0.000 | 4.280 | 0.000 | 3.610 |
| 3205 | H89D_F116P_A117M | 0.000 | 5.330 | 0.000 | 5.460 | 0.000 | 4.370 |
| 3206 | H89D_F116P_D123I | 0.000 | 2.080 | 0.110 | 1.480 | 1.000 | 0.660 |
| 3207 | H89D_P90F | 0.000 | 4.550 | 0.000 | 4.000 | 0.000 | 3.510 |
| 3208 | H89D_P90F_A117M | 0.000 | 5.330 | 0.000 | 4.910 | 0.000 | 5.410 |
| 3209 | H89D_P90F_A117M_D123I | 0.000 | 3.680 | 0.000 | 3.020 | 0.000 | 2.400 |
| 3210 | H89D_P90F_F116P | 0.000 | 4.420 | 0.000 | 4.370 | 0.000 | 3.420 |
| 3211 | H89D_P90F_F116P_A117M | 0.000 | 6.960 | 0.000 | 6.820 | 0.000 | 5.910 |
| 3212 | H89D_P90F_F116P_A117M_D123I | 0.000 | 4.230 | 0.000 | 3.870 | 0.000 | 3.330 |
| 3213 | H89D_P90F_R97F_A117M | 0.000 | 2.060 | 0.000 | 1.720 | 0.000 | 1.970 |
| 3214 | H89D_P90F_R97F_F116P_A117M | 0.000 | 1.960 | 0.000 | 2.470 | 0.000 | 2.320 |
| 3215 | H89D_P90F_V93K_F116P | 0.000 | 2.750 | 0.000 | 2.710 | 0.000 | 2.220 |
| 3216 | H89D_P90F_V93K_F116P_A117M | 0.000 | 2.280 | 0.000 | 1.200 | 1.000 | 1.050 |
| 3217 | H89D_P90F_V93K_P94D_F116P_A117M | 0.010 | 2.080 | 0.000 | 1.800 | 0.000 | 2.090 |
| 3218 | H89D_R97F_A117M | 0.000 | 2.030 | 0.000 | 2.370 | 0.000 | 2.580 |
| 3219 | H89D_V93K | 0.000 | 2.550 | 0.000 | 2.600 | 0.000 | 1.860 |
| 3220 | H89D_V93K_A117M | 0.000 | 3.520 | 0.000 | 3.620 | 0.000 | 2.510 |
| 3221 | H89D_V93K_P94D_A117M | 0.000 | 2.390 | 0.000 | 1.700 | 0.000 | 1.980 |
| 97 | I16W | 0.000 | 8.200 | 0.000 | 7.580 | 0.000 | 8.540 |
| 3222 | I16W_A117M | 0.000 | 7.180 | 0.000 | 6.650 | 0.000 | 8.100 |
| 3223 | I16W_A117M_D123I | 0.000 | 2.910 | 0.000 | 2.790 | 0.000 | 2.360 |
| 3224 | I16W_D123I | 0.000 | 4.900 | 0.000 | 4.280 | 0.000 | 4.870 |
| 3225 | I16W_F116P | 0.000 | 9.630 | 0.000 | 9.810 | 0.000 | 9.960 |
| 3226 | I16W_F116P_A117M | 0.000 | 9.490 | 0.000 | 9.400 | 0.000 | 9.500 |
| 3227 | I16W_F116P_A117M_D123I | 0.000 | 6.170 | 0.000 | 5.990 | 0.000 | 4.980 |
| 3228 | I16W_F116P_A117M_R119Q | 0.000 | 3.820 | 0.000 | 3.400 | 0.000 | 3.010 |
| 3229 | I16W_F116P_A117M_R119Q_D123I | 0.000 | 3.570 | 0.000 | 3.620 | 0.000 | 2.320 |
| 3230 | I16W_F116P_D123I | 0.000 | 6.860 | 0.000 | 6.880 | 0.000 | 5.990 |
| 3231 | I16W_F116P_R119Q | 0.000 | 4.760 | 0.000 | 4.590 | 0.000 | 3.440 |
| 3232 | I16W_F116P_R119Q_D123I | 0.000 | 5.780 | 0.000 | 5.870 | 0.000 | 5.260 |
| 3233 | I16W_H89D | 0.000 | 3.430 | 0.000 | 2.840 | 0.000 | 4.110 |
| 3234 | I16W_H89D_F116P | 0.000 | 6.620 | 0.000 | 6.610 | 0.000 | 5.990 |
| 3235 | I16W_H89D_F116P_A117M | 0.000 | 4.660 | 0.000 | 4.680 | 0.000 | 3.970 |
| 3236 | I16W_H89D_P90F_F116P | 0.000 | 4.930 | 0.000 | 5.020 | 0.000 | 4.140 |
| 1348 | I16W_P36H | 0.000 | 4.870 | 0.000 | 4.750 | 0.000 | 3.330 |
| 3237 | I16W_P36H_F116P | 0.000 | 8.660 | 0.000 | 8.490 | 0.000 | 7.210 |
| 3238 | I16W_P36H_F116P_A117M | 0.000 | 8.320 | 0.000 | 8.250 | 0.000 | 7.390 |
| 3239 | I16W_P36H_F116P_D123I | 0.000 | 3.540 | 0.000 | 3.560 | 0.000 | 2.540 |
| 3240 | I16W_P36H_H89D_F116P | 0.000 | 4.390 | 0.000 | 5.040 | 0.000 | 3.780 |
| 3241 | I16W_P36H_H89D_F116P_A117M | 0.000 | 2.590 | 0.000 | 2.260 | 0.000 | 1.680 |
| 3242 | I16W_P36H_P90F_F116P | 0.000 | 5.080 | 0.000 | 4.930 | 0.000 | 4.060 |
| 3243 | I16W_P36H_P94D_F116P | 0.000 | 2.140 | 0.000 | 2.000 | 0.000 | 2.000 |
| 3244 | I16W_P36H_R97F_F116P | 0.000 | 4.300 | 0.000 | 4.390 | 0.000 | 3.910 |
| 3245 | I16W_P36H_R97F_F116P_A117M | 0.000 | 4.970 | 0.000 | 4.730 | 0.000 | 3.820 |
| 3246 | I16W_P36H_V93K_F116P | 0.000 | 2.010 | 0.000 | 2.440 | 0.000 | 0.890 |
| 3247 | I16W_P36H_V93K_P94D_F116P | 0.000 | 3.480 | 0.000 | 3.250 | 0.000 | 1.920 |
| 3248 | I16W_P36H_V93K_R97F_F116P | 0.000 | 2.180 | 0.000 | 1.190 | 1.000 | 1.020 |
| 1361 | I16W_P90F | 0.000 | 2.690 | 0.000 | 2.390 | 0.000 | 2.620 |
| 3249 | I16W_P90F_A117M | 0.000 | 1.150 | 0.000 | 1.380 | 0.000 | 2.060 |
| 3250 | I16W_P90F_F116P | 0.000 | 8.530 | 0.000 | 8.230 | 0.000 | 8.300 |
| 3251 | I16W_P90F_F116P_A117M | 0.000 | 5.980 | 0.000 | 5.870 | 0.000 | 5.280 |
| 3252 | I16W_P90F_F116P_D123I | 0.000 | 2.270 | 0.000 | 1.480 | 0.000 | 1.810 |
| 3253 | I16W_P90F_P94D_F116P | 0.000 | 4.660 | 0.000 | 3.900 | 0.000 | 4.220 |
| 3254 | I16W_P90F_P94D_R97F_F116P | 0.000 | 2.240 | 0.000 | 1.920 | 0.000 | 1.420 |
| 3255 | I16W_P90F_R97F_F116P | 0.000 | 5.960 | 0.000 | 5.430 | 0.000 | 4.830 |
| 3256 | I16W_P90F_R97F_F116P_A117M | 0.000 | 2.860 | 0.000 | 2.730 | 0.000 | 2.270 |
| 3257 | I16W_P90F_V93K_F116P | 0.000 | 4.190 | 0.000 | 3.850 | 0.000 | 3.300 |
| 3258 | I16W_P90F_V93K_P94D_F116P | 0.000 | 4.580 | 0.000 | 4.750 | 0.000 | 4.150 |
| 3259 | I16W_P90F_V93K_P94D_R97F_F116P | 0.000 | 2.740 | 0.000 | 2.640 | 0.000 | 2.170 |
| 3260 | I16W_P90F_V93K_R97F_F116P | 0.000 | 3.260 | 0.070 | 1.710 | 0.000 | 2.160 |
| 3261 | I16W_P94D | 0.000 | 2.150 | 0.000 | 2.660 | 0.000 | 2.310 |
| 3262 | I16W_P94D_F116P | 0.000 | 3.790 | 0.000 | 4.160 | 0.000 | 3.650 |
| 3263 | I16W_P94D_F116P_A117M | 0.000 | 3.820 | 0.000 | 3.630 | 0.000 | 3.540 |
| 3264 | I16W_P94D_F116P_D123I | 0.000 | 2.470 | 0.000 | 2.930 | 0.000 | 2.510 |
| 1385 | I16W_R97F | 0.000 | 4.560 | 0.000 | 4.640 | 0.000 | 3.780 |
| 3265 | I16W_R97F_A117M | 0.000 | 3.940 | 0.000 | 3.470 | 0.000 | 3.160 |
| 3266 | I16W_R97F_D123I | 0.000 | 3.520 | 0.000 | 2.910 | 0.000 | 3.300 |
| 3267 | I16W_R97F_F116P | 0.000 | 6.000 | 0.000 | 6.180 | 0.000 | 5.680 |
| 3268 | I16W_R97F_F116P_A117M | 0.000 | 6.370 | 0.000 | 6.340 | 0.000 | 5.930 |
| 3269 | I16W_R97F_F116P_A117M_D123I | 0.000 | 4.390 | 0.000 | 4.100 | 0.000 | 3.230 |
| 3270 | I16W_R97F_F116P_D123I | 0.000 | 5.040 | 0.000 | 4.460 | 0.000 | 4.210 |
| 3271 | I16W_R97F_F116P_R119Q_D123I | 0.000 | 3.010 | 0.000 | 2.360 | 0.000 | 2.360 |
| 3272 | I16W_V93K_F116P | 0.000 | 6.870 | 0.000 | 7.080 | 0.000 | 6.570 |
| 3273 | I16W_V93K_F116P_A117M | 0.000 | 4.640 | 0.000 | 4.430 | 0.000 | 3.510 |
| 3274 | I16W_V93K_F116P_A117M_R119Q | 0.000 | 3.070 | 0.000 | 2.490 | 0.000 | 1.650 |
| 3275 | I16W_V93K_F116P_R119Q | 0.000 | 4.970 | 0.000 | 4.760 | 0.000 | 3.640 |
| 3276 | I16W_V93K_F116P_R119Q_D123I | 0.000 | 3.150 | 0.000 | 2.900 | 0.000 | 2.310 |
| 3277 | I16W_V93K_P94D_F116P | 0.000 | 4.760 | 0.000 | 4.420 | 0.000 | 3.690 |
| 3278 | I16W_V93K_P94D_R97F_F116P | 0.000 | 3.150 | 0.000 | 2.890 | 0.000 | 2.510 |
| 3279 | I16W_V93K_R97F_F116P | 0.000 | 4.390 | 0.000 | 4.360 | 0.000 | 3.470 |
| 3280 | I16W_V93K_R97F_F116P_A117M | 0.000 | 2.270 | 0.000 | 2.230 | 0.080 | 1.380 |
| 3281 | I16W_V93K_R97F_F116P_R119Q | 0.000 | 3.310 | 0.000 | 2.590 | 0.000 | 2.650 |
| 3282 | I16W_Y37D_F116P_D123I | 0.000 | 2.330 | 0.000 | 2.060 | 1.000 | 1.580 |
| 3283 | P14E_I16W_P90F_F116P | 0.000 | 3.330 | 0.000 | 3.240 | 0.000 | 2.260 |
| 3284 | P14E_P36H | 0.000 | 2.060 | 0.000 | 1.930 | 0.010 | 1.430 |
| 3285 | P14E_P36H_P90F_F116P_A117M | 0.000 | 1.930 | 0.000 | 2.050 | 0.000 | 1.640 |
| 3286 | P14E_P90F | 0.000 | 3.110 | 0.000 | 2.970 | 0.000 | 1.960 |
| 3287 | P14E_P90F_A117M | 0.000 | 3.860 | 0.000 | 3.080 | 0.000 | 3.440 |
| 3288 | P14E_P90F_F116P | 0.000 | 3.820 | 0.000 | 3.720 | 0.000 | 2.860 |
| 3289 | P14E_P90F_F116P_A117M | 0.000 | 5.890 | 0.000 | 5.860 | 0.000 | 5.340 |
| 3290 | P14E_P90F_F116P_A117M_D123I | 0.000 | 3.290 | 0.000 | 2.810 | 0.000 | 1.970 |
| 107 | P36H | 0.000 | 6.370 | 0.000 | 6.500 | 0.000 | 4.540 |
| 3291 | P36H_A117M | 0.000 | 7.210 | 0.000 | 7.280 | 0.000 | 5.920 |
| 3292 | P36H_A117M_D123I | 0.000 | 5.320 | 0.000 | 5.350 | 0.000 | 4.440 |
| 3293 | P36H_D123I | 0.000 | 2.050 | 0.000 | 2.400 | 0.000 | 1.800 |
| 3294 | P36H_F116P | 0.000 | 6.380 | 0.000 | 6.480 | 0.000 | 4.580 |
| 3295 | P36H_F116P_A117M | 0.000 | 7.220 | 0.000 | 6.900 | 0.000 | 5.210 |
| 3296 | P36H_F116P_A117M_D123I | 0.000 | 3.260 | 0.000 | 3.900 | 0.000 | 2.110 |
| 3297 | P36H_F116P_D123I | 0.000 | 2.590 | 0.000 | 2.560 | 0.000 | 1.780 |
| 3298 | P36H_H89D | 0.000 | 4.320 | 0.000 | 4.120 | 0.000 | 2.780 |
| 3299 | P36H_H89D_A117M | 0.000 | 4.920 | 0.000 | 5.680 | 0.000 | 3.810 |
| 3300 | P36H_H89D_F116P | 0.000 | 2.980 | 0.000 | 2.960 | 0.850 | 1.520 |
| 3301 | P36H_H89D_F116P_A117M | 0.000 | 3.010 | 0.000 | 3.100 | 0.040 | 1.400 |
| 3302 | P36H_H89D_P90F_A117M | 0.000 | 2.430 | 0.000 | 1.340 | 0.000 | 1.440 |
| 3303 | P36H_H89D_P90F_F116P | 0.000 | 2.630 | 0.000 | 1.870 | 0.000 | 1.430 |
| 3304 | P36H_H89D_P90F_F116P_A117M | 0.000 | 3.210 | 0.000 | 3.440 | 0.000 | 2.300 |
| 1476 | P36H_P90F | 0.000 | 5.280 | 0.000 | 5.030 | 0.000 | 3.910 |
| 3305 | P36H_P90F_A117M | 0.000 | 6.420 | 0.000 | 6.630 | 0.000 | 4.940 |
| 3306 | P36H_P90F_A117M_D123I | 0.000 | 2.870 | 0.000 | 3.160 | 0.000 | 2.270 |
| 3307 | P36H_P90F_F116P | 0.000 | 5.540 | 0.000 | 5.520 | 0.000 | 3.200 |
| 3308 | P36H_P90F_F116P_A117M | 0.000 | 6.450 | 0.000 | 6.350 | 0.000 | 4.910 |
| 3309 | P36H_P90F_F116P_A117M_D123I | 0.000 | 4.680 | 0.000 | 4.050 | 0.000 | 3.000 |
| 3310 | P36H_P90F_P94D_A117M | 0.000 | 1.880 | 0.000 | 1.640 | 0.000 | 2.070 |
| 3311 | P36H_P90F_R97F_A117M | 0.000 | 4.570 | 0.000 | 4.530 | 0.000 | 3.640 |
| 3312 | P36H_P90F_R97F_A117M_D123I | 0.000 | 2.420 | 0.300 | 1.210 | 0.000 | 1.090 |
| 3313 | P36H_P90F_R97F_F116P_A117M | 0.000 | 3.580 | 0.000 | 3.310 | 0.000 | 3.120 |
| 3314 | P36H_P90F_R97F_F116P_A117M_D123I | 0.000 | 2.400 | 0.000 | 2.540 | 0.000 | 1.590 |
| 3315 | P36H_P94D_A117M | 0.000 | 2.240 | 0.000 | 2.000 | 0.000 | 2.240 |
| 3316 | P36H_P94D_A117M_D123I | 0.000 | 1.930 | 0.000 | 1.490 | 0.000 | 2.160 |
| 1502 | P36H_R97F | 0.000 | 2.670 | 0.000 | 3.020 | 0.000 | 2.430 |
| 3317 | P36H_R97F_A117M | 0.000 | 4.580 | 0.000 | 4.480 | 0.000 | 3.500 |
| 3318 | P36H_R97F_A117M_D123I | 0.000 | 4.320 | 0.000 | 4.180 | 0.000 | 3.120 |
| 3319 | P36H_R97F_F116P | 0.000 | 2.580 | 0.000 | 2.130 | 0.000 | 2.430 |
| 3320 | P36H_R97F_F116P_A117M | 0.000 | 3.840 | 0.000 | 3.640 | 0.000 | 2.810 |
| 1510 | P36H_V93K | 0.000 | 3.000 | 0.000 | 2.730 | 0.050 | 1.720 |
| 3321 | P36H_V93K_A117M | 0.000 | 1.870 | 0.000 | 2.110 | 0.160 | 1.220 |
| 3322 | P36H_V93K_F116P | 0.000 | 2.880 | 0.000 | 2.470 | 0.710 | 1.280 |
| 3323 | P36H_V93K_P94D | 0.000 | 2.980 | 0.000 | 3.290 | 0.000 | 1.500 |
| 3324 | P36H_V93K_P94D_A117M | 0.000 | 3.430 | 0.000 | 3.550 | 0.000 | 2.320 |
| 3325 | P36H_V93K_P94D_F116P | 0.000 | 2.430 | 0.000 | 2.550 | 0.000 | 1.940 |
| 3326 | P36H_V93K_R97F_A117M | 0.000 | 1.960 | 0.000 | 2.210 | 0.000 | 1.490 |
| 134 | P90F | 0.000 | 7.030 | 0.000 | 6.730 | 0.000 | 7.020 |
| 3327 | P90F_A117M | 0.000 | 8.570 | 0.000 | 8.600 | 0.000 | 9.160 |
| 3328 | P90F_A117M_D123I | 0.000 | 7.850 | 0.000 | 7.630 | 0.000 | 6.580 |
| 3329 | P90F_A117M_R119Q | 0.000 | 3.360 | 0.000 | 3.110 | 0.000 | 2.420 |
| 3330 | P90F_A117M_R119Q_D123I | 0.000 | 5.990 | 0.000 | 5.630 | 0.000 | 4.340 |
| 3331 | P90F_D123I | 0.000 | 4.000 | 0.000 | 3.750 | 0.000 | 2.690 |
| 3332 | P90F_F116P | 0.000 | 7.820 | 0.000 | 8.000 | 0.000 | 6.810 |
| 3333 | P90F_F116P_A117M | 0.000 | 9.240 | 0.000 | 9.400 | 0.000 | 8.800 |
| 3334 | P90F_F116P_A117M_D123I | 0.000 | 7.780 | 0.000 | 7.600 | 0.000 | 7.110 |
| 3335 | P90F_F116P_A117M_R119Q | 0.000 | 4.510 | 0.000 | 4.670 | 0.000 | 3.750 |
| 3336 | P90F_F116P_A117M_R119Q_D123I | 0.000 | 6.580 | 0.000 | 6.760 | 0.000 | 5.540 |
| 3337 | P90F_F116P_D123I | 0.000 | 4.590 | 0.000 | 4.350 | 0.000 | 3.190 |
| 3338 | P90F_F116P_R119Q_D123I | 0.000 | 3.330 | 0.000 | 3.690 | 0.000 | 2.540 |
| 3339 | P90F_P94D | 0.000 | 2.160 | 0.000 | 2.240 | 0.000 | 1.260 |
| 3340 | P90F_P94D_A117M | 0.000 | 3.610 | 0.000 | 3.520 | 0.000 | 3.070 |
| 3341 | P90F_P94D_A117M_D123I | 0.000 | 3.100 | 0.000 | 3.270 | 0.000 | 2.510 |
| 3342 | P90F_P94D_F116P | 0.000 | 2.040 | 0.000 | 2.730 | 0.000 | 1.940 |
| 3343 | P90F_P94D_F116P_A117M | 0.000 | 4.170 | 0.000 | 4.680 | 0.000 | 3.970 |
| 3344 | P90F_P94D_F116P_A117M_D123I | 0.000 | 3.950 | 0.000 | 3.450 | 0.000 | 3.790 |
| 3345 | P90F_P94D_R97F_F116P_A117M_D123I | 0.000 | 2.560 | 0.000 | 2.210 | 0.000 | 2.200 |
| 1623 | P90F_R97F | 0.000 | 3.290 | 0.000 | 3.670 | 0.000 | 2.750 |
| 3346 | P90F_R97F_A117M | 0.000 | 6.280 | 0.000 | 6.010 | 0.000 | 5.210 |
| 3347 | P90F_R97F_A117M_D123I | 0.000 | 5.350 | 0.000 | 5.410 | 0.000 | 4.660 |
| 3348 | P90F_R97F_A117M_R119Q_D123I | 0.000 | 3.430 | 0.010 | 2.240 | 0.000 | 2.130 |
| 3349 | P90F_R97F_D123I | 0.000 | 2.050 | 0.000 | 2.370 | 1.000 | 0.910 |
| 3350 | P90F_R97F_F116P | 0.000 | 4.470 | 0.000 | 4.680 | 0.000 | 3.460 |
| 3351 | P90F_R97F_F116P_A117M | 0.000 | 6.810 | 0.000 | 6.200 | 0.000 | 5.980 |
| 3352 | P90F_R97F_F116P_A117M_D123I | 0.000 | 5.830 | 0.000 | 5.600 | 0.000 | 4.980 |
| 3353 | P90F_R97F_F116P_A117M_R119Q_D123I | 0.000 | 3.500 | 0.000 | 2.960 | 0.000 | 2.860 |
| 3354 | P90F_R97F_F116P_D123I | 0.000 | 2.080 | 0.000 | 2.060 | 0.000 | 1.900 |
| 3355 | P90F_R119Q_D123I | 0.000 | 2.270 | 0.010 | 1.730 | 0.020 | 1.290 |
| 1630 | P90F_V93K | 0.000 | 3.820 | 0.000 | 3.860 | 0.000 | 2.380 |
| 3356 | P90F_V93K_A117M | 0.000 | 4.390 | 0.000 | 5.140 | 0.000 | 3.550 |
| 3357 | P90F_V93K_F116P | 0.000 | 5.660 | 0.000 | 5.980 | 0.000 | 4.520 |
| 3358 | P90F_V93K_F116P_A117M | 0.000 | 7.250 | 0.000 | 7.340 | 0.000 | 5.800 |
| 3359 | P90F_V93K_F116P_A117M_D123I | 0.000 | 2.660 | 0.010 | 2.250 | 0.070 | 1.390 |
| 3360 | P90F_V93K_F116P_A117M_R119Q | 0.000 | 4.740 | 0.000 | 5.410 | 0.000 | 3.350 |
| 3361 | P90F_V93K_F116P_A117M_R119Q_D123I | 0.000 | 4.940 | 0.000 | 4.560 | 0.000 | 3.470 |
| 3362 | P90F_V93K_P94D | 0.000 | 3.150 | 0.000 | 3.240 | 0.000 | 2.060 |
| 3363 | P90F_V93K_P94D_A117M | 0.000 | 2.770 | 0.000 | 2.800 | 0.000 | 2.520 |
| 3364 | P90F_V93K_P94D_F116P | 0.000 | 3.860 | 0.000 | 3.970 | 0.000 | 3.200 |
| 3365 | P90F_V93K_P94D_F116P_A117M | 0.000 | 5.200 | 0.000 | 5.290 | 0.000 | 4.800 |
| 3366 | P90F_V93K_P94D_F116P_A117M_R119Q | 0.000 | 2.510 | 0.000 | 1.810 | 0.020 | 1.440 |
| 3367 | P90F_V93K_P94D_F116P_A117M_R119Q_D123I | 0.000 | 3.610 | 0.000 | 3.620 | 0.000 | 3.000 |
| 3368 | P90F_V93K_P94D_R97F_A117M | 0.000 | 2.260 | 0.000 | 2.080 | 0.000 | 1.710 |
| 3369 | P90F_V93K_P94D_R97F_F116P | 0.000 | 2.730 | 0.000 | 2.390 | 0.120 | 1.620 |
| 3370 | P90F_V93K_P94D_R97F_F116P_A117M | 0.000 | 3.860 | 0.000 | 3.880 | 0.000 | 3.520 |
| 3371 | P90F_V93K_P94D_R97F_F116P_A117M_R119Q_D123I | 0.000 | 2.320 | 0.000 | 1.990 | 0.000 | 2.050 |
| 1632 | P90F_V93K_R97F | 0.000 | 2.360 | 0.000 | 1.790 | 0.000 | 1.150 |
| 3372 | P90F_V93K_R97F_A117M | 0.000 | 2.150 | 0.000 | 3.130 | 0.000 | 2.420 |
| 3373 | P90F_V93K_R97F_F116P_A117M | 0.000 | 5.080 | 0.000 | 4.580 | 0.000 | 4.200 |
| 3374 | P90F_V93K_R97F_F116P_A117M_D123I | 0.000 | 2.270 | 0.000 | 2.180 | 0.590 | 0.990 |
| 3375 | P90F_V93K_R97F_F116P_A117M_R119Q | 0.000 | 3.180 | 0.000 | 2.380 | 0.000 | 1.720 |
| 3376 | P90F_V93K_R97F_F116P_A117M_R119Q_D123I | 0.000 | 3.980 | 0.000 | 3.140 | 0.000 | 2.740 |
| 3377 | P94D_A117M | 0.000 | 2.570 | 1.000 | 0.490 | 0.000 | 1.680 |
| 3378 | P94D_A117M_D123I | 0.000 | 3.710 | 0.000 | 2.900 | 0.000 | 3.020 |
| 3379 | P94D_F116P | 0.000 | 1.720 | 0.000 | 2.170 | 0.000 | 1.850 |
| 3380 | P94D_F116P_A117M | 0.000 | 2.050 | 0.000 | 2.060 | 0.000 | 1.870 |
| 3381 | P94D_R97F_A117M_D123I | 0.000 | 1.780 | 0.000 | 2.060 | 0.000 | 2.040 |
| 186 | R97F | 0.000 | 2.490 | 0.610 | 0.390 | 1.000 | 0.400 |
| 3382 | R97F_A117M | 0.000 | 5.220 | 0.000 | 4.730 | 0.000 | 5.570 |
| 3383 | R97F_A117M_D123I | 0.000 | 6.240 | 0.000 | 5.860 | 0.000 | 5.230 |
| 3384 | R97F_A117M_R119Q | 0.000 | 2.300 | 0.000 | 2.790 | 0.020 | 2.320 |
| 3385 | R97F_A117M_R119Q_D123I | 0.000 | 3.740 | 0.000 | 3.480 | 0.000 | 3.290 |
| 3386 | R97F_D123I | 0.000 | 2.270 | 0.000 | 1.180 | 0.030 | 1.190 |
| 3387 | R97F_F116P | 0.000 | 3.240 | 0.000 | 3.870 | 0.000 | 2.620 |
| 3388 | R97F_F116P_A117M | 0.000 | 4.610 | 0.000 | 4.360 | 0.000 | 3.300 |
| 3389 | R97F_F116P_A117M_D123I | 0.000 | 3.150 | 0.000 | 3.840 | 0.000 | 2.840 |
| 3390 | R97F_F116P_D123I | 0.020 | 2.000 | 0.000 | 2.030 | 0.030 | 1.090 |
| 257 | R119Q | 0.000 | 1.830 | 0.000 | 1.910 | 0.000 | 2.030 |
| 3391 | R119Q_D123I | 0.000 | 2.890 | 0.000 | 0.930 | 0.040 | 1.220 |
| 158 | V93K | 0.000 | 4.920 | 0.000 | 4.750 | 0.000 | 4.770 |
| 3392 | V93K_A117M | 0.000 | 5.910 | 0.000 | 6.330 | 0.000 | 5.460 |
| 3393 | V93K_A117M_D123I | 0.000 | 3.260 | 0.000 | 3.160 | 0.000 | 2.150 |
| 3394 | V93K_A117M_R119Q | 0.000 | 5.280 | 0.000 | 5.340 | 0.000 | 3.620 |
| 3395 | V93K_A117M_R119Q_D123I | 0.000 | 4.920 | 0.000 | 4.620 | 0.000 | 3.500 |
| 3396 | V93K_F116P | 0.000 | 4.910 | 0.000 | 5.600 | 0.000 | 3.700 |
| 3397 | V93K_F116P_A117M | 0.000 | 4.690 | 0.000 | 4.570 | 0.000 | 3.420 |
| 3398 | V93K_F116P_A117M_R119Q_D123I | 0.000 | 2.140 | 0.110 | 1.210 | 0.040 | 1.140 |
| 3399 | V93K_F116P_R119Q_D123I | 0.000 | 2.220 | 1.000 | 0.610 | 1.000 | 0.770 |
| 3400 | V93K_P94D | 0.000 | 3.350 | 0.000 | 3.500 | 0.000 | 2.460 |
| 3401 | V93K_P94D_A117M | 0.000 | 4.720 | 0.000 | 4.760 | 0.000 | 3.620 |
| 3402 | V93K_P94D_A117M_R119Q | 0.000 | 3.430 | 0.000 | 2.880 | 0.170 | 1.940 |
| 3403 | V93K_P94D_A117M_R119Q_D123I | 0.000 | 3.140 | 0.000 | 3.250 | 0.000 | 2.430 |
| 3404 | V93K_P94D_F116P | 0.000 | 3.450 | 0.000 | 3.430 | 0.000 | 2.280 |
| 3405 | V93K_P94D_F116P_A117M | 0.000 | 2.570 | 0.000 | 2.840 | 0.000 | 1.690 |
| 3406 | V93K_P94D_R97F_A117M | 0.000 | 3.330 | 0.000 | 3.170 | 0.000 | 2.960 |
| 1724 | V93K_R97F | 0.000 | 2.140 | 0.000 | 2.160 | 0.000 | 2.200 |
| 3407 | V93K_R97F_A117M | 0.000 | 4.600 | 0.000 | 4.490 | 0.000 | 4.100 |
| 3408 | V93K_R97F_A117M_D123I | 0.000 | 2.280 | 0.020 | 2.190 | 0.280 | 1.360 |
| 3409 | V93K_R97F_A117M_R119Q | 0.000 | 3.850 | 0.000 | 4.340 | 0.000 | 2.960 |
| 3410 | V93K_R97F_A117M_R119Q_D123I | 0.000 | 3.460 | 0.000 | 3.340 | 0.000 | 3.240 |
| 3411 | V93K_R97F_F116P | 0.000 | 2.170 | 0.000 | 2.470 | 0.000 | 1.400 |
| 3412 | V93K_R97F_F116P_A117M | 0.000 | 2.450 | 0.000 | 2.390 | 0.000 | 1.890 |
| 1729 | V93K_R119Q | 0.000 | 2.120 | 0.000 | 2.190 | 0.000 | 1.660 |
| 3413 | V93K_R119Q_D123I | 0.000 | 2.240 | 0.000 | 2.560 | 0.240 | 1.390 |
| 3414 | Y7D_P90F_F116P | 0.000 | 2.210 | 0.050 | 1.420 | 0.000 | 1.250 |
| 3415 | Y37D_A117M_D123I | 0.000 | 3.860 | 0.000 | 4.160 | 0.000 | 5.150 |
| 3416 | Y37D_F116P_A117M_D123I | 0.000 | 2.210 | 0.000 | 2.170 | 0.190 | 1.500 |
| 3417 | Y37D_P90F_F116P_A117M_D123I | 0.000 | 3.780 | 0.000 | 2.590 | 0.000 | 2.440 |
| 3418 | Y37D_R97F_A117M_D123I | 0.000 | 2.880 | 0.000 | 2.450 | 0.000 | 2.390 |

*adjusted p-val under dU/dI primer tolerance condition
**fitness_combined score under dU/dI primer tolerance condition
^{†}adjusted p-val under dU primer tolerance condition
^{††}fitness_combined score under dU primer tolerance condition
^{‡}adjusted p-val under dUTP incorporation tolerance condition
^{‡‡}fitness_combined score under dUTP incorporation tolerance condition

### Additional Embodiments

1. A Thermococcus kodakarensis (KOD) polymerase variant, comprising one or more of the following substitutions:
an aspartate (D), glutamate (E), phenylalanine (F), histidine (H), lysine (K), leucine (L), asparagine (N), threonine (T), valine (V) or tryptophan (W) at a position corresponding to isoleucine 2 (I2) of SEQ ID NO: 1;
a D, H, I, serine (S), T, or tyrosine (Y) at a position corresponding to L3 of SEQ ID NO: 1;
an I, L, methionine (M), proline (P), or Y at a position corresponding to D4 of SEQ ID NO: 1;
a D, E, F, glycine (G), H, L, W, or Y at a position corresponding to T5 of SEQ ID NO: 1;
a L at a position corresponding to D6 of SEQ ID NO: 1;
an alanine (A), cysteine (C), D, E, F, G, H, I, K, L, M, N, P, glutamine (Q), arginine (R), S, T, V, or W at a position corresponding to Y7 of SEQ ID NO: 1;
a D, E, H, or T at a position corresponding to I8 of SEQ ID NO: 1;
an A, C, D, E, F, H, I, L, N, R, or Y at a position corresponding to T9 of SEQ ID NO: 1;
a P or Q at a position corresponding to E10 of SEQ ID NO: 1;
a Q or T at a position corresponding to D11 of SEQ ID NO: 1;
a C, Q, T, or V at a position corresponding to G12 of SEQ ID NO: 1;
a G, H, T, V, or W at a position corresponding to K13 of SEQ ID NO: 1;
a C, D, E, F, H, M, Q, S, T or W at a position corresponding to P14 of SEQ ID NO: 1;
a Q or W at a position corresponding to V15 of SEQ ID NO: 1;
an A, C, F, H, K, L, M, S, W, or Y at a position corresponding to I16 of SEQ ID NO: 1;
a P at a position corresponding to R17 of SEQ ID NO: 1;
a Y at a position corresponding to I18 of SEQ ID NO: 1;
a W at a position corresponding to E22 of SEQ ID NO: 1;
a D at a position corresponding to F34 of SEQ ID NO: 1;
an A, C, D, E, F, H, I, K, L, M, N, Q, R, S, T, V, W, or Y at a position corresponding to P36 of SEQ ID NO: 1;
an A, D, E, F, G, S, or T at a position corresponding to Y37 of SEQ ID NO: 1;
a L at a position corresponding to V71 of SEQ ID NO: 1;
a R at a position corresponding to F87 of SEQ ID NO: 1;
a D, P, or W at a position corresponding to H89 of SEQ ID NO: 1;
a C, D, E, F, G, H, I, K, L, M, N, Q, R, S, W, or Y at a position corresponding to P90 of SEQ ID NO: 1;
a D, E, N, or Y at a position corresponding to Q91 of SEQ ID NO: 1;
a P at a position corresponding to D92 of SEQ ID NO: 1;
an A, C, D, E, F, G, H, I, K, M, N, P, Q, R, S, T, W, or Y at a position corresponding to V93 of SEQ ID NO: 1;
a C, D, E, F, G, H, I, M, N, Q, W, or Y at a position corresponding to P94 of SEQ ID NO: 1;
a P and R at a position corresponding to I96 of SEQ ID NO: 1;
an A, C, D, E, F, G, H, I, K, L, M, N, P, Q, S, T, V, W, or Y at a position corresponding to R97 of SEQ ID NO: 1;
an E or Q at a position corresponding to R101 of SEQ ID NO: 1;
a D or F at a position corresponding to I109 of SEQ ID NO: 1;
a P at a position corresponding to Y110 of SEQ ID NO: 1;
a G at a position corresponding to E111 of SEQ ID NO: 1;
a D or E at a position corresponding to Y112 of SEQ ID NO: 1;
a P at a position corresponding to D113 of SEQ ID NO: 1;
an E, G, H, K, N, P, Q, S, or Y at a position corresponding to I114 of SEQ ID NO: 1;
a C, D, F, H, N, S, T, or Y at a position corresponding to P115 of SEQ ID NO: 1;
an A, D, E, G, H, I, K, N, P, Q, R, or V at a position corresponding to F116 of SEQ ID NO: 1;
a D, E, L, M, Q, or W at a position corresponding to A117 of SEQ ID NO: 1;
an A, C, D, E, F, G, H, I, K, L, M, N, P, Q, S, T, V, W, or Y at a position corresponding to R119 of SEQ ID NO: 1;
a P at a position corresponding to Y120 of SEQ ID NO: 1;
a K, R, or W at a position corresponding to I122 of SEQ ID NO: 1; or
an A, C, F, H, I, K, L, M, Q, R, S, T, V, W, or a Y at a position corresponding to D123 of SEQ ID NO: 1.
a N at a position corresponding to L138 of SEQ ID NO: 1;
an E or Q at a position corresponding to A139 of SEQ ID NO: 1;
a R at a position corresponding to F140 of SEQ ID NO: 1;
an A, C, G, H, K, N, P, Q, S, T, or V at a position corresponding to D141 of SEQ ID NO: 1;
a F, G, H, K, P, R, W, or Y at a position corresponding to I142 of SEQ ID NO: 1;
an A, C, F, G, H, I, K, L, M, N, P, Q, R, S, T, V, W, or Y at a position corresponding to E143 of SEQ ID NO: 1;
an A, D, E, F, G, H, K, L, M, P, Q, R, W, or Y at a position corresponding to T144 of SEQ ID NO: 1;
a G at a position corresponding to L145 of SEQ ID NO: 1;
a D or E at a position corresponding to Y146 of SEQ ID NO: 1;
a L at a position corresponding to G149 of SEQ ID NO: 1;
an A, D, E, N, or S at a position corresponding to F152 of SEQ ID NO: 1;
a P or Y at a position corresponding to E154 of SEQ ID NO: 1;
an I or V at a position corresponding to G155 of SEQ ID NO: 1;
an E, F, R, W, or Y at a position corresponding to I157 of SEQ ID NO: 1;
a D, H, K, Q, R, W, or Y at a position corresponding to L158 of SEQ ID NO: 1;
an E, G, P, or R at a position corresponding to M159 of SEQ ID NO: 1;
a F at a position corresponding to I160 of SEQ ID NO: 1;
a D, E, F, H, K, L, M, N, P, R, T, W, or Y at a position corresponding to S161 of SEQ ID NO: 1;
a P at a position corresponding to Y162 of SEQ ID NO: 1;
a C, E, F, G, N, or T at a position corresponding to A168 of SEQ ID NO: 1;
an A, G, H, R, S, W, or Y at a position corresponding to V170 of SEQ ID NO: 1;
a R at a position corresponding to T172 of SEQ ID NO: 1;
a W at a position corresponding to E187 of SEQ ID NO: 1;
a F or Y at a position corresponding to I191 of SEQ ID NO: 1;
a G at a position corresponding to F194 of SEQ ID NO: 1;
a F at a position corresponding to K199 of SEQ ID NO: 1;
an E, F, H, I, K, N, Q, R, V, W, or Y at a position corresponding to T208 of SEQ ID NO: 1;
an A, C, E, G, H, I, L, M, N, P, S, T, V, or W at a position corresponding to Y209 of SEQ ID NO: 1;
a C, D, E, G, M, P, Q, S, or T at a position corresponding to N210 of SEQ ID NO: 1;
a C, D, E, F, H, I, K, L, M, N, P, Q, R, S, T, V, W or Y at a position corresponding to G211 of SEQ ID NO: 1;
a F, I, K, L, M, P, Q, R, V, W, or Y at a position corresponding to D212 of SEQ ID NO: 1;
a D or E at a position corresponding to N213 of SEQ ID NO: 1;
an A, C, D, E, G, H, I, K, L, M, N, P, Q, R, S, T, V, or W at a position corresponding to F214 of SEQ ID NO: 1;
an A, C, F, G, H, I, K, L, M, N, P, Q, R, S, T, V, W, or Y at a position corresponding to D215 of SEQ ID NO: 1;
an A or D at a position corresponding to F216 of SEQ ID NO: 1;
an A, D, E, G, L, P, S, T, or W at a position corresponding to Y218 of SEQ ID NO: 1;
a P, R, or V at a position corresponding to L219 of SEQ ID NO: 1;
an I or V at a position corresponding to K220 of SEQ ID NO: 1;
an A, E, or P at a position corresponding to C223 of SEQ ID NO: 1;
a W or Y at a position corresponding to K240 of SEQ ID NO: 1;
a D at a position corresponding to A249 of SEQ ID NO: 1;
a L at a position corresponding to V250 of SEQ ID NO: 1;
a F, G, N, or R at a position corresponding to E251 of SEQ ID NO: 1;
a C, E, M, T, or W at a position corresponding to V252 of SEQ ID NO: 1;
a H, N, or R at a position corresponding to G254 of SEQ ID NO: 1;
a F, W, or Y at a position corresponding to H257 of SEQ ID NO: 1;
an A, C, F, G, H, I, K, L, M, N, P, Q, R, S, T, V, W, or Y at a position corresponding to D259 of SEQ ID NO: 1;
a W at a position corresponding to L260 of SEQ ID NO: 1;
a D, E, or F at a position corresponding to Y261 of SEQ ID NO: 1;
a D or P at a position corresponding to Y273 of SEQ ID NO: 1;
a D at a position corresponding to T274 of SEQ ID NO: 1;
a H, P, R, or S at a position corresponding to L275 of SEQ ID NO: 1;
an E or P at a position corresponding to A277 of SEQ ID NO: 1;
a F at a position corresponding to V278 of SEQ ID NO: 1;
a M at a position corresponding to E280 of SEQ ID NO: 1;
a W at a position corresponding to A281 of SEQ ID NO: 1;
a D, E, F, I, M, P, T, or V at a position corresponding to K287 of SEQ ID NO: 1;
an A, D, E, F, G, H, I, L, M, P, Q, R, S, T, V, W, or Y at a position corresponding to K289 of SEQ ID NO: 1;
an A, D, E, G, H, K, N, P, Q, R, S, or T at a position corresponding to V290 of SEQ ID NO: 1;
a D, E, N, Q, or T at a position corresponding to A292 of SEQ ID NO: 1;
a K, P, or R at a position corresponding to E294 of SEQ ID NO: 1;
an A, C, D, E, G, H, K, N, P, Q, R, S, T, W, or Y at a position corresponding to I295 of SEQ ID NO: 1;
a D or P at a position corresponding to A298 of SEQ ID NO: 1;
a D, E, G, H, I, K, L, M, N, P, Q, R, T, or V at a position corresponding to W299 of SEQ ID NO: 1;
a P at a position corresponding to E300 of SEQ ID NO: 1;
a P at a position corresponding to T301 of SEQ ID NO: 1;
a P at a position corresponding to G302 of SEQ ID NO: 1;
a H at a position corresponding to L305 of SEQ ID NO: 1;
a D, F, H, K, L, N, Q, R, W, or Y at a position corresponding to V308 of SEQ ID NO: 1;
a D, P, or R at a position corresponding to A309 of SEQ ID NO: 1;
an A, C, D, E, F, G, H, I, K, L, M, N, P, Q, R, S, T, V, or W at a position corresponding to Y311 of SEQ ID NO: 1;
a D, E, F, H, I, K, L, M, N, P, Q, R, V, W, or Y at a position corresponding to S312 of SEQ ID NO: 1;
a P or Q at a position corresponding to M313 of SEQ ID NO: 1;
a P at a position corresponding to E314 of SEQ ID NO: 1;
an A, C, F, G, I, K, L, M, N, P, Q, R, S, T, V, W, or Y at a position corresponding to D315 of SEQ ID NO: 1;
a D, E, F, H, M, N, Q, R, W, or Y at a position corresponding to A316 of SEQ ID NO: 1;
a P at a position corresponding to K317 of SEQ ID NO: 1;
a P or R at a position corresponding to V318 of SEQ ID NO: 1; and
a D, I, K, L, M, Q, R, or V at a position corresponding to T319 of SEQ ID NO: 1.

2. The KOD polymerase variant of embodiment 1, comprising a D, E, F, H, K, L, N, T, V, or W substitution at a position corresponding to I2 SEQ ID NO: 1.

2b. The KOD polymerase variant of embodiment 1, comprising a D substitution at a position corresponding to I2 SEQ ID NO: 1.

2c. The KOD polymerase variant of embodiment 1, comprising an E substitution at a position corresponding to I2 SEQ ID NO: 1.

2d. The KOD polymerase variant of embodiment 1, comprising a F substitution at a position corresponding to I2 SEQ ID NO: 1.

2e. The KOD polymerase variant of embodiment 1, comprising a H substitution at a position corresponding to I2 SEQ ID NO: 1.

2f. The KOD polymerase variant of embodiment 1, comprising a K substitution at a position corresponding to I2 SEQ ID NO: 1.

2g. The KOD polymerase variant of embodiment 1, comprising a L substitution at a position corresponding to I2 SEQ ID NO: 1.

2h. The KOD polymerase variant of embodiment 1, comprising a N substitution at a position corresponding to I2 SEQ ID NO: 1.

2i. The KOD polymerase variant of embodiment 1, comprising a T substitution at a position corresponding to I2 SEQ ID NO: 1.

2j. The KOD polymerase variant of embodiment 1, comprising a V substitution at a position corresponding to I2 SEQ ID NO: 1.

2k. The KOD polymerase variant of embodiment 1, comprising a W substitution at a position corresponding to I2 SEQ ID NO: 1.

3. The KOD polymerase variant of any one of the preceding embodiments, comprising a D, H, I S, T, or Y substitution at a position corresponding to L3 of SEQ ID NO: 1.

3b. The KOD polymerase variant of any one of the preceding embodiments, comprising a D substitution at a position corresponding to L3 SEQ ID NO: 1.

3c. The KOD polymerase variant of any one of the preceding embodiments, comprising a H substitution at a position corresponding to L3 SEQ ID NO: 1.

3d. The KOD polymerase variant of any one of the preceding embodiments, comprising an I substitution at a position corresponding to L3 SEQ ID NO: 1.

3e. The KOD polymerase variant of any one of the preceding embodiments, comprising a S substitution at a position corresponding to L3 SEQ ID NO: 1.

3f. The KOD polymerase variant of any one of the preceding embodiments, comprising a T substitution at a position corresponding to L3 SEQ ID NO: 1.

3g. The KOD polymerase variant of any one of the preceding embodiments, comprising a Y substitution at a position corresponding to L3 SEQ ID NO: 1.

4. The KOD polymerase variant of any one of the preceding embodiments, comprising an I, L, M, P, or Y substitution at a position corresponding to D4 of SEQ ID NO: 1.

4b. The KOD polymerase variant of any one of the preceding embodiments, comprising an I substitution at a position corresponding to D4 SEQ ID NO: 1.

4c. The KOD polymerase variant of any one of the preceding embodiments, comprising a L substitution at a position corresponding to D4 SEQ ID NO: 1.

4d. The KOD polymerase variant of any one of the preceding embodiments, comprising a M substitution at a position corresponding to D4 SEQ ID NO: 1.

4e. The KOD polymerase variant of any one of the preceding embodiments, comprising a P substitution at a position corresponding to D4 SEQ ID NO: 1.

4f. The KOD polymerase variant of any one of the preceding embodiments, comprising a Y substitution at a position corresponding to D4 SEQ ID NO: 1.

5. The KOD polymerase variant of any one of the preceding embodiments, comprising a D, E, F, G, H, L, W, or Y substitution at a position corresponding to T5 of SEQ ID NO: 1.

5b. The KOD polymerase variant of any one of the preceding embodiments, comprising a D substitution at a position corresponding to T5 SEQ ID NO: 1.

5c. The KOD polymerase variant of any one of the preceding embodiments, comprising an E substitution at a position corresponding to T5 SEQ ID NO: 1.

5d. The KOD polymerase variant of any one of the preceding embodiments, comprising a F substitution at a position corresponding to T5 SEQ ID NO: 1.

5e. The KOD polymerase variant of any one of the preceding embodiments, comprising a G substitution at a position corresponding to T5 SEQ ID NO: 1.

5f. The KOD polymerase variant of any one of the preceding embodiments, comprising a H substitution at a position corresponding to T5 SEQ ID NO: 1.

5g. The KOD polymerase variant of any one of the preceding embodiments, comprising a L substitution at a position corresponding to T5 SEQ ID NO: 1.

5h. The KOD polymerase variant of any one of the preceding embodiments, comprising a W substitution at a position corresponding to T5 SEQ ID NO: 1.

5i. The KOD polymerase variant of any one of the preceding embodiments, comprising a Y substitution at a position corresponding to T5 SEQ ID NO: 1.

6. The KOD polymerase variant of any one of the preceding embodiments, comprising a L substitution at a position corresponding to D6 of SEQ ID NO: 1.

7. The KOD polymerase variant of any one of the preceding embodiments, comprising an A, C, D, E, F, G, H, I, K, L, M, N, P, Q, R, S, T, V, or W substitution at a position corresponding to Y7 of SEQ ID NO: 1.

7b. The KOD polymerase variant of any one of the preceding embodiments, comprising an A substitution at a position corresponding to Y7 SEQ ID NO: 1.

7c. The KOD polymerase variant of any one of the preceding embodiments, comprising a C substitution at a position corresponding to Y7 SEQ ID NO: 1.

7d. The KOD polymerase variant of any one of the preceding embodiments, comprising a D substitution at a position corresponding to Y7 SEQ ID NO: 1.

7e. The KOD polymerase variant of any one of the preceding embodiments, comprising an E substitution at a position corresponding to Y7 SEQ ID NO: 1.

7f. The KOD polymerase variant of any one of the preceding embodiments, comprising a F substitution at a position corresponding to Y7 SEQ ID NO: 1.

7g. The KOD polymerase variant of any one of the preceding embodiments, comprising a G substitution at a position corresponding to Y7 SEQ ID NO: 1.

7h. The KOD polymerase variant of any one of the preceding embodiments, comprising a H substitution at a position corresponding to Y7 SEQ ID NO: 1.

7i. The KOD polymerase variant of any one of the preceding embodiments, comprising an I substitution at a position corresponding to Y7 SEQ ID NO: 1.

7j. The KOD polymerase variant of any one of the preceding embodiments, comprising a K substitution at a position corresponding to Y7 SEQ ID NO: 1.

7k. The KOD polymerase variant of any one of the preceding embodiments, comprising a L substitution at a position corresponding to Y7 SEQ ID NO: 1.

7l. The KOD polymerase variant of any one of the preceding embodiments, comprising a M substitution at a position corresponding to Y7 SEQ ID NO: 1.

7m. The KOD polymerase variant of any one of the preceding embodiments, comprising a N substitution at a position corresponding to Y7 SEQ ID NO: 1.

7n. The KOD polymerase variant of any one of the preceding embodiments, comprising a P substitution at a position corresponding to Y7 SEQ ID NO: 1.

7o. The KOD polymerase variant of any one of the preceding embodiments, comprising a Q substitution at a position corresponding to Y7 SEQ ID NO: 1.

7p. The KOD polymerase variant of any one of the preceding embodiments, comprising a R substitution at a position corresponding to Y7 SEQ ID NO: 1.

7q. The KOD polymerase variant of any one of the preceding embodiments, comprising a S substitution at a position corresponding to Y7 SEQ ID NO: 1.

7r. The KOD polymerase variant of any one of the preceding embodiments, comprising a T substitution at a position corresponding to Y7 SEQ ID NO: 1.

7s. The KOD polymerase variant of any one of the preceding embodiments, comprising a V substitution at a position corresponding to Y7 SEQ ID NO: 1.

7t. The KOD polymerase variant of any one of the preceding embodiments, comprising a W substitution at a position corresponding to Y7 SEQ ID NO: 1.

8. The KOD polymerase variant of any one of the preceding embodiments, comprising a D, E, H, or T substitution at a position corresponding to I8 of SEQ ID NO: 1.

8b. The KOD polymerase variant of any one of the preceding embodiments, comprising a D substitution at a position corresponding to I8 SEQ ID NO: 1.

8c. The KOD polymerase variant of any one of the preceding embodiments, comprising an E substitution at a position corresponding to I8 SEQ ID NO: 1.

8d. The KOD polymerase variant of any one of the preceding embodiments, comprising a H substitution at a position corresponding to I8 SEQ ID NO: 1.

8e. The KOD polymerase variant of any one of the preceding embodiments, comprising a T substitution at a position corresponding to I8 SEQ ID NO: 1.

9. The KOD polymerase variant of any one of the preceding embodiments, comprising an A, C, D, E, F, H, I, L, N, R, or Y substitution at a position corresponding to T9 of SEQ ID NO: 1.

9b. The KOD polymerase variant of any one of the preceding embodiments, comprising an A substitution at a position corresponding to T9 SEQ ID NO: 1.

9c. The KOD polymerase variant of any one of the preceding embodiments, comprising a C substitution at a position corresponding to T9 SEQ ID NO: 1.

9d. The KOD polymerase variant of any one of the preceding embodiments, comprising a D substitution at a position corresponding to T9 SEQ ID NO: 1.

9e. The KOD polymerase variant of any one of the preceding embodiments, comprising an E substitution at a position corresponding to T9 SEQ ID NO: 1.

9f. The KOD polymerase variant of any one of the preceding embodiments, comprising a F substitution at a position corresponding to T9 SEQ ID NO: 1.

9g. The KOD polymerase variant of any one of the preceding embodiments, comprising a H substitution at a position corresponding to T9 SEQ ID NO: 1.

9h. The KOD polymerase variant of any one of the preceding embodiments, comprising an I substitution at a position corresponding to T9 SEQ ID NO: 1.

9i. The KOD polymerase variant of any one of the preceding embodiments, comprising a L substitution at a position corresponding to T9 SEQ ID NO: 1.

9j. The KOD polymerase variant of any one of the preceding embodiments, comprising a N substitution at a position corresponding to T9 SEQ ID NO: 1.

9k. The KOD polymerase variant of any one of the preceding embodiments, comprising a R substitution at a position corresponding to T9 SEQ ID NO: 1.

9l. The KOD polymerase variant of any one of the preceding embodiments, comprising a Y substitution at a position corresponding to T9 SEQ ID NO: 1.

10. The KOD polymerase variant of any one of the preceding embodiments, comprising a P or Q substitution at a position corresponding to E10 of SEQ ID NO: 1.

10b. The KOD polymerase variant of any one of the preceding embodiments, comprising a P substitution at a position corresponding to E10 SEQ ID NO: 1.

10c. The KOD polymerase variant of any one of the preceding embodiments, comprising a Q substitution at a position corresponding to E10 SEQ ID NO: 1.

11. The KOD polymerase variant of any one of the preceding embodiments, comprising a Q or T substitution at a position corresponding to D11 of SEQ ID NO: 1.

11b. The KOD polymerase variant of any one of the preceding embodiments, comprising a Q substitution at a position corresponding to D11 SEQ ID NO: 1.

11c. The KOD polymerase variant of any one of the preceding embodiments, comprising a T substitution at a position corresponding to D11 SEQ ID NO: 1.

12. The KOD polymerase variant of any one of the preceding embodiments, comprising a C, Q, T, or V substitution at a position corresponding to G12 of SEQ ID NO: 1.

12b. The KOD polymerase variant of any one of the preceding embodiments, comprising a C substitution at a position corresponding to G12 SEQ ID NO: 1.

12c. The KOD polymerase variant of any one of the preceding embodiments, comprising a Q substitution at a position corresponding to G12 SEQ ID NO: 1.

12d. The KOD polymerase variant of any one of the preceding embodiments, comprising a T substitution at a position corresponding to G12 SEQ ID NO: 1.

12e. The KOD polymerase variant of any one of the preceding embodiments, comprising a V substitution at a position corresponding to G12 SEQ ID NO: 1.

13. The KOD polymerase variant of any one of the preceding embodiments, comprising a G, H, T, V, or W substitution at a position corresponding to K13 of SEQ ID NO: 1.

13b. The KOD polymerase variant of any one of the preceding embodiments, comprising a G substitution at a position corresponding to K13 SEQ ID NO: 1.

13c. The KOD polymerase variant of any one of the preceding embodiments, comprising a H substitution at a position corresponding to K13 SEQ ID NO: 1.

13d. The KOD polymerase variant of any one of the preceding embodiments, comprising a T substitution at a position corresponding to K13 SEQ ID NO: 1.

13e. The KOD polymerase variant of any one of the preceding embodiments, comprising a V substitution at a position corresponding to K13 SEQ ID NO: 1.

13f. The KOD polymerase variant of any one of the preceding embodiments, comprising a W substitution at a position corresponding to K13 SEQ ID NO: 1.

14. The KOD polymerase variant of any one of the preceding embodiments, comprising a C, D, E, F, H, M, Q, S, T or W substitution at a position corresponding to P14 of SEQ ID NO: 1.

14b. The KOD polymerase variant of any one of the preceding embodiments, comprising a C substitution at a position corresponding to P14 SEQ ID NO: 1.

14c. The KOD polymerase variant of any one of the preceding embodiments, comprising a D substitution at a position corresponding to P14 SEQ ID NO: 1.

14d. The KOD polymerase variant of any one of the preceding embodiments, comprising an E substitution at a position corresponding to P14 SEQ ID NO: 1.

14e. The KOD polymerase variant of any one of the preceding embodiments, comprising a F substitution at a position corresponding to P14 SEQ ID NO: 1.

14f. The KOD polymerase variant of any one of the preceding embodiments, comprising a H substitution at a position corresponding to P14 SEQ ID NO: 1.

14g. The KOD polymerase variant of any one of the preceding embodiments, comprising a M substitution at a position corresponding to P14 SEQ ID NO: 1.

14h. The KOD polymerase variant of any one of the preceding embodiments, comprising a Q substitution at a position corresponding to P14 SEQ ID NO: 1.

14i. The KOD polymerase variant of any one of the preceding embodiments, comprising a S substitution at a position corresponding to P14 SEQ ID NO: 1.

14j. The KOD polymerase variant of any one of the preceding embodiments, comprising a T substitution at a position corresponding to P14 SEQ ID NO: 1.

14k. The KOD polymerase variant of any one of the preceding embodiments, comprising a W substitution at a position corresponding to P14 SEQ ID NO: 1.

15. The KOD polymerase variant of any one of the preceding embodiments, comprising a Q or W substitution at a position corresponding to V15 of SEQ ID NO: 1.

15b. The KOD polymerase variant of any one of the preceding embodiments, comprising a Q substitution at a position corresponding to V15 SEQ ID NO: 1.

15c. The KOD polymerase variant of any one of the preceding embodiments, comprising a W substitution at a position corresponding to V15 SEQ ID NO: 1.

16. The KOD polymerase variant of any one of the preceding embodiments, comprising an A, C, F, H, K, L, M, S, W, or Y substitution at a position corresponding to I16 of SEQ ID NO: 1.

16b. The KOD polymerase variant of any one of the preceding embodiments, comprising an A substitution at a position corresponding to I16 SEQ ID NO: 1.

16c. The KOD polymerase variant of any one of the preceding embodiments, comprising a C substitution at a position corresponding to I16 SEQ ID NO: 1.

16d. The KOD polymerase variant of any one of the preceding embodiments, comprising a F substitution at a position corresponding to I16 SEQ ID NO: 1.

16e. The KOD polymerase variant of any one of the preceding embodiments, comprising a H substitution at a position corresponding to I16 SEQ ID NO: 1.

16f. The KOD polymerase variant of any one of the preceding embodiments, comprising a K substitution at a position corresponding to I16 SEQ ID NO: 1.

16g. The KOD polymerase variant of any one of the preceding embodiments, comprising a L substitution at a position corresponding to I16 SEQ ID NO: 1.

16h. The KOD polymerase variant of any one of the preceding embodiments, comprising a M substitution at a position corresponding to I16 SEQ ID NO: 1.

16i. The KOD polymerase variant of any one of the preceding embodiments, comprising a S substitution at a position corresponding to I16 SEQ ID NO: 1.

16j. The KOD polymerase variant of any one of the preceding embodiments, comprising a W substitution at a position corresponding to I16 SEQ ID NO: 1.

16k. The KOD polymerase variant of any one of the preceding embodiments, comprising a Y substitution at a position corresponding to I16 SEQ ID NO: 1.

17. The KOD polymerase variant of any one of the preceding embodiments, comprising a P substitution at a position corresponding to R17 of SEQ ID NO: 1.

18. The KOD polymerase variant of any one of the preceding embodiments, comprising a Y substitution at a position corresponding to I18 of SEQ ID NO: 1.

19. The KOD polymerase variant of any one of the preceding embodiments, comprising a W substitution at a position corresponding to E22 of SEQ ID NO: 1.

20. The KOD polymerase variant of any one of the preceding embodiments, comprising a D substitution at a position corresponding to F34 of SEQ ID NO: 1.

21. The KOD polymerase variant of any one of the preceding embodiments, comprising an A, C, D, E, F, H, I, K, L, M, N, Q, R, S, T, V, W, or Y substitution at a position corresponding to P36 of SEQ ID NO: 1.

21b. The KOD polymerase variant of any one of the preceding embodiments, comprising an A substitution at a position corresponding to P36 SEQ ID NO: 1.

21c. The KOD polymerase variant of any one of the preceding embodiments, comprising a C substitution at a position corresponding to P36 SEQ ID NO: 1.

21d. The KOD polymerase variant of any one of the preceding embodiments, comprising a D substitution at a position corresponding to P36 SEQ ID NO: 1.

21e. The KOD polymerase variant of any one of the preceding embodiments, comprising an E substitution at a position corresponding to P36 SEQ ID NO: 1.

21f. The KOD polymerase variant of any one of the preceding embodiments, comprising a F substitution at a position corresponding to P36 SEQ ID NO: 1.

21g. The KOD polymerase variant of any one of the preceding embodiments, comprising a H substitution at a position corresponding to P36 SEQ ID NO: 1.

21h. The KOD polymerase variant of any one of the preceding embodiments, comprising an I substitution at a position corresponding to P36 SEQ ID NO: 1.

21i. The KOD polymerase variant of any one of the preceding embodiments, comprising a K substitution at a position corresponding to P36 SEQ ID NO: 1.

21j. The KOD polymerase variant of any one of the preceding embodiments, comprising a L substitution at a position corresponding to P36 SEQ ID NO: 1.

21k. The KOD polymerase variant of any one of the preceding embodiments, comprising a M substitution at a position corresponding to P36 SEQ ID NO: 1.

21l. The KOD polymerase variant of any one of the preceding embodiments, comprising a N substitution at a position corresponding to P36 SEQ ID NO: 1.

21m. The KOD polymerase variant of any one of the preceding embodiments, comprising a Q substitution at a position corresponding to P36 SEQ ID NO: 1.

21n. The KOD polymerase variant of any one of the preceding embodiments, comprising a R substitution at a position corresponding to P36 SEQ ID NO: 1.

21o. The KOD polymerase variant of any one of the preceding embodiments, comprising a S substitution at a position corresponding to P36 SEQ ID NO: 1.

21p. The KOD polymerase variant of any one of the preceding embodiments, comprising a T substitution at a position corresponding to P36 SEQ ID NO: 1.

21q. The KOD polymerase variant of any one of the preceding embodiments, comprising a V substitution at a position corresponding to P36 SEQ ID NO: 1.

21r. The KOD polymerase variant of any one of the preceding embodiments, comprising a W substitution at a position corresponding to P36 SEQ ID NO: 1.

21s. The KOD polymerase variant of any one of the preceding embodiments, comprising a Y substitution at a position corresponding to P36 SEQ ID NO: 1.

22. The KOD polymerase variant of any one of the preceding embodiments, comprising an A, D, E, F, G, S, or T substitution at a position corresponding to Y37 of SEQ ID NO: 1.

22b. The KOD polymerase variant of any one of the preceding embodiments, comprising an A substitution at a position corresponding to Y37 SEQ ID NO: 1.

22c. The KOD polymerase variant of any one of the preceding embodiments, comprising a D substitution at a position corresponding to Y37 SEQ ID NO: 1.

22d. The KOD polymerase variant of any one of the preceding embodiments, comprising an E substitution at a position corresponding to Y37 SEQ ID NO: 1.

22e. The KOD polymerase variant of any one of the preceding embodiments, comprising a F substitution at a position corresponding to Y37 SEQ ID NO: 1.

22f. The KOD polymerase variant of any one of the preceding embodiments, comprising a G substitution at a position corresponding to Y37 SEQ ID NO: 1.

22g. The KOD polymerase variant of any one of the preceding embodiments, comprising a S substitution at a position corresponding to Y37 SEQ ID NO: 1.

22h. The KOD polymerase variant of any one of the preceding embodiments, comprising a T substitution at a position corresponding to Y37 SEQ ID NO: 1.

23. The KOD polymerase variant of any one of the preceding embodiments, comprising a L substitution at a position corresponding to V71 of SEQ ID NO: 1.

24. The KOD polymerase variant of any one of the preceding embodiments, comprising a R substitution at a position corresponding to F87 of SEQ ID NO: 1.

25. The KOD polymerase variant of any one of the preceding embodiments, comprising a D, P, or W substitution at a position corresponding to H89 of SEQ ID NO: 1.

25b. The KOD polymerase variant of any one of the preceding embodiments, comprising a D substitution at a position corresponding to H89 SEQ ID NO: 1.

25c. The KOD polymerase variant of any one of the preceding embodiments, comprising a P substitution at a position corresponding to H89 SEQ ID NO: 1.

25d. The KOD polymerase variant of any one of the preceding embodiments, comprising a W substitution at a position corresponding to H89 SEQ ID NO: 1.

26. The KOD polymerase variant of any one of the preceding embodiments, comprising a C, D, E, F, G, H, I, K, L, M, N, Q, R, S, W, or Y substitution at a position corresponding to P90 of SEQ ID NO: 1.

26b. The KOD polymerase variant of any one of the preceding embodiments, comprising a C substitution at a position corresponding to P90 SEQ ID NO: 1.

26c. The KOD polymerase variant of any one of the preceding embodiments, comprising a D substitution at a position corresponding to P90 SEQ ID NO: 1.

26d. The KOD polymerase variant of any one of the preceding embodiments, comprising an E substitution at a position corresponding to P90 SEQ ID NO: 1.

26e. The KOD polymerase variant of any one of the preceding embodiments, comprising a F substitution at a position corresponding to P90 SEQ ID NO: 1.

26f. The KOD polymerase variant of any one of the preceding embodiments, comprising a G substitution at a position corresponding to P90 SEQ ID NO: 1.

26g. The KOD polymerase variant of any one of the preceding embodiments, comprising a H substitution at a position corresponding to P90 SEQ ID NO: 1.

26h. The KOD polymerase variant of any one of the preceding embodiments, comprising an I substitution at a position corresponding to P90 SEQ ID NO: 1.

26i. The KOD polymerase variant of any one of the preceding embodiments, comprising a K substitution at a position corresponding to P90 SEQ ID NO: 1.

26j. The KOD polymerase variant of any one of the preceding embodiments, comprising a L substitution at a position corresponding to P90 SEQ ID NO: 1.

26k. The KOD polymerase variant of any one of the preceding embodiments, comprising a M substitution at a position corresponding to P90 SEQ ID NO: 1.

26l. The KOD polymerase variant of any one of the preceding embodiments, comprising a N substitution at a position corresponding to P90 SEQ ID NO: 1.

26m. The KOD polymerase variant of any one of the preceding embodiments, comprising a Q substitution at a position corresponding to P90 SEQ ID NO: 1.

26n. The KOD polymerase variant of any one of the preceding embodiments, comprising a R substitution at a position corresponding to P90 SEQ ID NO: 1.

26o. The KOD polymerase variant of any one of the preceding embodiments, comprising a S substitution at a position corresponding to P90 SEQ ID NO: 1.

26p. The KOD polymerase variant of any one of the preceding embodiments, comprising a W substitution at a position corresponding to P90 SEQ ID NO: 1.

26q. The KOD polymerase variant of any one of the preceding embodiments, comprising a Y substitution at a position corresponding to P90 SEQ ID NO: 1.

27. The KOD polymerase variant of any one of the preceding embodiments, comprising a D, E, N, or Y substitution at a position corresponding to Q91 of SEQ ID NO: 1.

27b. The KOD polymerase variant of any one of the preceding embodiments, comprising a D substitution at a position corresponding to Q91 SEQ ID NO: 1.

27c. The KOD polymerase variant of any one of the preceding embodiments, comprising an E substitution at a position corresponding to Q91 SEQ ID NO: 1.

27d. The KOD polymerase variant of any one of the preceding embodiments, comprising a N substitution at a position corresponding to Q91 SEQ ID NO: 1.

27e. The KOD polymerase variant of any one of the preceding embodiments, comprising a Y substitution at a position corresponding to Q91 SEQ ID NO: 1.

28. The KOD polymerase variant of any one of the preceding embodiments, comprising a P substitution at a position corresponding to D92 of SEQ ID NO: 1.

29. The KOD polymerase variant of any one of the preceding embodiments, comprising an A, C, D, E, F, G, H, I, K, M, N, P, Q, R, S, T, W, or Y substitution at a position corresponding to V93 of SEQ ID NO: 1.

29b. The KOD polymerase variant of any one of the preceding embodiments, comprising an A substitution at a position corresponding to V93 SEQ ID NO: 1.

29c. The KOD polymerase variant of any one of the preceding embodiments, comprising a C substitution at a position corresponding to V93 SEQ ID NO: 1.

29d. The KOD polymerase variant of any one of the preceding embodiments, comprising a D substitution at a position corresponding to V93 SEQ ID NO: 1.

29e. The KOD polymerase variant of any one of the preceding embodiments, comprising an E substitution at a position corresponding to V93 SEQ ID NO: 1.

29f. The KOD polymerase variant of any one of the preceding embodiments, comprising a F substitution at a position corresponding to V93 SEQ ID NO: 1.

29g. The KOD polymerase variant of any one of the preceding embodiments, comprising a G substitution at a position corresponding to V93 SEQ ID NO: 1.

29h. The KOD polymerase variant of any one of the preceding embodiments, comprising a H substitution at a position corresponding to V93 SEQ ID NO: 1.

29i. The KOD polymerase variant of any one of the preceding embodiments, comprising an I substitution at a position corresponding to V93 SEQ ID NO: 1.

29j. The KOD polymerase variant of any one of the preceding embodiments, comprising a K substitution at a position corresponding to V93 SEQ ID NO: 1.

29k. The KOD polymerase variant of any one of the preceding embodiments, comprising a M substitution at a position corresponding to V93 SEQ ID NO: 1.

29l. The KOD polymerase variant of any one of the preceding embodiments, comprising a N substitution at a position corresponding to V93 SEQ ID NO: 1.

29m. The KOD polymerase variant of any one of the preceding embodiments, comprising a P substitution at a position corresponding to V93 SEQ ID NO: 1.

29n. The KOD polymerase variant of any one of the preceding embodiments, comprising a Q substitution at a position corresponding to V93 SEQ ID NO: 1.

29o. The KOD polymerase variant of any one of the preceding embodiments, comprising a R substitution at a position corresponding to V93 SEQ ID NO: 1.

29p. The KOD polymerase variant of any one of the preceding embodiments, comprising a S substitution at a position corresponding to V93 SEQ ID NO: 1.

29q. The KOD polymerase variant of any one of the preceding embodiments, comprising a T substitution at a position corresponding to V93 SEQ ID NO: 1.

29r. The KOD polymerase variant of any one of the preceding embodiments, comprising a W substitution at a position corresponding to V93 SEQ ID NO: 1.

29s. The KOD polymerase variant of any one of the preceding embodiments, comprising a Y substitution at a position corresponding to V93 SEQ ID NO: 1.

30. The KOD polymerase variant of any one of the preceding embodiments, comprising a C, D, E, F, G, H, I, M, N, Q, W, or Y substitution at a position corresponding to P94 of SEQ ID NO: 1.

30b. The KOD polymerase variant of any one of the preceding embodiments, comprising a C substitution at a position corresponding to P94 SEQ ID NO: 1.

30c. The KOD polymerase variant of any one of the preceding embodiments, comprising a D substitution at a position corresponding to P94 SEQ ID NO: 1.

30d. The KOD polymerase variant of any one of the preceding embodiments, comprising an E substitution at a position corresponding to P94 SEQ ID NO: 1.

30e. The KOD polymerase variant of any one of the preceding embodiments, comprising a F substitution at a position corresponding to P94 SEQ ID NO: 1.

30f. The KOD polymerase variant of any one of the preceding embodiments, comprising a G substitution at a position corresponding to P94 SEQ ID NO: 1.

30g. The KOD polymerase variant of any one of the preceding embodiments, comprising a H substitution at a position corresponding to P94 SEQ ID NO: 1.

30h. The KOD polymerase variant of any one of the preceding embodiments, comprising an I substitution at a position corresponding to P94 SEQ ID NO: 1.

30i. The KOD polymerase variant of any one of the preceding embodiments, comprising a M substitution at a position corresponding to P94 SEQ ID NO: 1.

30j. The KOD polymerase variant of any one of the preceding embodiments, comprising a N substitution at a position corresponding to P94 SEQ ID NO: 1.

30k. The KOD polymerase variant of any one of the preceding embodiments, comprising a Q substitution at a position corresponding to P94 SEQ ID NO: 1.

30l. The KOD polymerase variant of any one of the preceding embodiments, comprising a W substitution at a position corresponding to P94 SEQ ID NO: 1.

30m. The KOD polymerase variant of any one of the preceding embodiments, comprising a Y substitution at a position corresponding to P94 SEQ ID NO: 1.

31. The KOD polymerase variant of any one of the preceding embodiments, comprising a P or R substitution at a position corresponding to I96 of SEQ ID NO: 1.

31b. The KOD polymerase variant of any one of the preceding embodiments, comprising a P substitution at a position corresponding to I96 SEQ ID NO: 1.

31c. The KOD polymerase variant of any one of the preceding embodiments, comprising a R substitution at a position corresponding to I96 SEQ ID NO: 1.

32. The KOD polymerase variant of any one of the preceding embodiments, comprising an A, C, D, E, F, G, H, I, K, L, M, N, P, Q, S, T, V, W, or Y substitution at a position corresponding to R97 of SEQ ID NO: 1.

32b. The KOD polymerase variant of any one of the preceding embodiments, comprising an A substitution at a position corresponding to R97 SEQ ID NO: 1.

32c. The KOD polymerase variant of any one of the preceding embodiments, comprising a C substitution at a position corresponding to R97 SEQ ID NO: 1.

32d. The KOD polymerase variant of any one of the preceding embodiments, comprising a D substitution at a position corresponding to R97 SEQ ID NO: 1.

32e. The KOD polymerase variant of any one of the preceding embodiments, comprising an E substitution at a position corresponding to R97 SEQ ID NO: 1.

32f. The KOD polymerase variant of any one of the preceding embodiments, comprising a F substitution at a position corresponding to R97 SEQ ID NO: 1.

32g. The KOD polymerase variant of any one of the preceding embodiments, comprising a G substitution at a position corresponding to R97 SEQ ID NO: 1.

32h. The KOD polymerase variant of any one of the preceding embodiments, comprising a H substitution at a position corresponding to R97 SEQ ID NO: 1.

32i. The KOD polymerase variant of any one of the preceding embodiments, comprising an I substitution at a position corresponding to R97 SEQ ID NO: 1.

32j. The KOD polymerase variant of any one of the preceding embodiments, comprising a K substitution at a position corresponding to R97 SEQ ID NO: 1.

32k. The KOD polymerase variant of any one of the preceding embodiments, comprising a L substitution at a position corresponding to R97 SEQ ID NO: 1.

32l. The KOD polymerase variant of any one of the preceding embodiments, comprising a M substitution at a position corresponding to R97 SEQ ID NO: 1.

32m. The KOD polymerase variant of any one of the preceding embodiments, comprising a N substitution at a position corresponding to R97 SEQ ID NO: 1.

32n. The KOD polymerase variant of any one of the preceding embodiments, comprising a P substitution at a position corresponding to R97 SEQ ID NO: 1.

32o. The KOD polymerase variant of any one of the preceding embodiments, comprising a Q substitution at a position corresponding to R97 SEQ ID NO: 1.

32p. The KOD polymerase variant of any one of the preceding embodiments, comprising a S substitution at a position corresponding to R97 SEQ ID NO: 1.

32q. The KOD polymerase variant of any one of the preceding embodiments, comprising a T substitution at a position corresponding to R97 SEQ ID NO: 1.

32r. The KOD polymerase variant of any one of the preceding embodiments, comprising a V substitution at a position corresponding to R97 SEQ ID NO: 1.

32s. The KOD polymerase variant of any one of the preceding embodiments, comprising a W substitution at a position corresponding to R97 SEQ ID NO: 1.

32t. The KOD polymerase variant of any one of the preceding embodiments, comprising a Y substitution at a position corresponding to R97 SEQ ID NO: 1.

33. The KOD polymerase variant of any one of the preceding embodiments, comprising an E or Q substitution at a position corresponding to R101 of SEQ ID NO: 1.

33b. The KOD polymerase variant of any one of the preceding embodiments, comprising an E substitution at a position corresponding to R101 SEQ ID NO: 1.

33c. The KOD polymerase variant of any one of the preceding embodiments, comprising a Q substitution at a position corresponding to R101 SEQ ID NO: 1.

34. The KOD polymerase variant of any one of the preceding embodiments, comprising a D or F substitution at a position corresponding to I109 of SEQ ID NO: 1.

34b. The KOD polymerase variant of any one of the preceding embodiments, comprising a D substitution at a position corresponding to I109 SEQ ID NO: 1.

34c. The KOD polymerase variant of any one of the preceding embodiments, comprising a F substitution at a position corresponding to I109 SEQ ID NO: 1.

35. The KOD polymerase variant of any one of the preceding embodiments, comprising a P substitution at a position corresponding to Y110 of SEQ ID NO: 1.

36. The KOD polymerase variant of any one of the preceding embodiments, comprising a G substitution at a position corresponding to E111 of SEQ ID NO: 1.

37. The KOD polymerase variant of any one of the preceding embodiments, comprising a D or E substitution at a position corresponding to Y112 of SEQ ID NO: 1.

37b. The KOD polymerase variant of any one of the preceding embodiments, comprising a D substitution at a position corresponding to Y112 SEQ ID NO: 1.

37c. The KOD polymerase variant of any one of the preceding embodiments, comprising an E substitution at a position corresponding to Y112 SEQ ID NO: 1.

38. The KOD polymerase variant of any one of the preceding embodiments, comprising a P substitution at a position corresponding to D113 of SEQ ID NO: 1.

39. The KOD polymerase variant of any one of the preceding embodiments, comprising an E, G, H, K, N, P, Q, S, or Y substitution at a position corresponding to I114 of SEQ ID NO: 1.

39b. The KOD polymerase variant of any one of the preceding embodiments, comprising an E substitution at a position corresponding to I114 SEQ ID NO: 1.

39c. The KOD polymerase variant of any one of the preceding embodiments, comprising a G substitution at a position corresponding to I114 SEQ ID NO: 1.

39d. The KOD polymerase variant of any one of the preceding embodiments, comprising a H substitution at a position corresponding to I114 SEQ ID NO: 1.

39e. The KOD polymerase variant of any one of the preceding embodiments, comprising a K substitution at a position corresponding to I114 SEQ ID NO: 1.

39f. The KOD polymerase variant of any one of the preceding embodiments, comprising a N substitution at a position corresponding to I114 SEQ ID NO: 1.

39g. The KOD polymerase variant of any one of the preceding embodiments, comprising a P substitution at a position corresponding to I114 SEQ ID NO: 1.

39h. The KOD polymerase variant of any one of the preceding embodiments, comprising a Q substitution at a position corresponding to I114 SEQ ID NO: 1.

39i. The KOD polymerase variant of any one of the preceding embodiments, comprising a S substitution at a position corresponding to I114 SEQ ID NO: 1.

39j. The KOD polymerase variant of any one of the preceding embodiments, comprising a Y substitution at a position corresponding to I114 SEQ ID NO: 1.

40. The KOD polymerase variant of any one of the preceding embodiments, comprising a C, D, F, H, N, S, T, or Y substitution at a position corresponding to P115 of SEQ ID NO: 1.

40b. The KOD polymerase variant of any one of the preceding embodiments, comprising a C substitution at a position corresponding to P115 SEQ ID NO: 1.

40c. The KOD polymerase variant of any one of the preceding embodiments, comprising a D substitution at a position corresponding to P115 SEQ ID NO: 1.

40d. The KOD polymerase variant of any one of the preceding embodiments, comprising a F substitution at a position corresponding to P115 SEQ ID NO: 1.

40e. The KOD polymerase variant of any one of the preceding embodiments, comprising a H substitution at a position corresponding to P115 SEQ ID NO: 1.

40f. The KOD polymerase variant of any one of the preceding embodiments, comprising a N substitution at a position corresponding to P115 SEQ ID NO: 1.

40g. The KOD polymerase variant of any one of the preceding embodiments, comprising a S substitution at a position corresponding to P115 SEQ ID NO: 1.

40h. The KOD polymerase variant of any one of the preceding embodiments, comprising a T substitution at a position corresponding to P115 SEQ ID NO: 1.

40i. The KOD polymerase variant of any one of the preceding embodiments, comprising a Y substitution at a position corresponding to P115 SEQ ID NO: 1.

41. The KOD polymerase variant of any one of the preceding embodiments, comprising an A, D, E, G, H, I, K, N, P, Q, R, or V substitution at a position corresponding to F116 of SEQ ID NO: 1.

41b. The KOD polymerase variant of any one of the preceding embodiments, comprising an A substitution at a position corresponding to F116 SEQ ID NO: 1.

41c. The KOD polymerase variant of any one of the preceding embodiments, comprising a D substitution at a position corresponding to F116 SEQ ID NO: 1.

41d. The KOD polymerase variant of any one of the preceding embodiments, comprising an E substitution at a position corresponding to F116 SEQ ID NO: 1.

41e. The KOD polymerase variant of any one of the preceding embodiments, comprising a G substitution at a position corresponding to F116 SEQ ID NO: 1.

41f. The KOD polymerase variant of any one of the preceding embodiments, comprising a H substitution at a position corresponding to F116 SEQ ID NO: 1.

41g. The KOD polymerase variant of any one of the preceding embodiments, comprising an I substitution at a position corresponding to F116 SEQ ID NO: 1.

41h. The KOD polymerase variant of any one of the preceding embodiments, comprising a K substitution at a position corresponding to F116 SEQ ID NO: 1.

41i. The KOD polymerase variant of any one of the preceding embodiments, comprising a N substitution at a position corresponding to F116 SEQ ID NO: 1.

41j. The KOD polymerase variant of any one of the preceding embodiments, comprising a P substitution at a position corresponding to F116 SEQ ID NO: 1.

41k. The KOD polymerase variant of any one of the preceding embodiments, comprising a Q substitution at a position corresponding to F116 SEQ ID NO: 1.

41l. The KOD polymerase variant of any one of the preceding embodiments, comprising a R substitution at a position corresponding to F116 SEQ ID NO: 1.

41m. The KOD polymerase variant of any one of the preceding embodiments, comprising a V substitution at a position corresponding to F116 SEQ ID NO: 1.

42. The KOD polymerase variant of any one of the preceding embodiments, comprising a D, E, L, M, Q, or W substitution at a position corresponding to A117 of SEQ ID NO: 1.

42b. The KOD polymerase variant of any one of the preceding embodiments, comprising a D substitution at a position corresponding to A117 SEQ ID NO: 1.

42c. The KOD polymerase variant of any one of the preceding embodiments, comprising an E substitution at a position corresponding to A117 SEQ ID NO: 1.

42d. The KOD polymerase variant of any one of the preceding embodiments, comprising a L substitution at a position corresponding to A117 SEQ ID NO: 1.

42e. The KOD polymerase variant of any one of the preceding embodiments, comprising a M substitution at a position corresponding to A117 SEQ ID NO: 1.

42f. The KOD polymerase variant of any one of the preceding embodiments, comprising a Q substitution at a position corresponding to A117 SEQ ID NO: 1.

42g. The KOD polymerase variant of any one of the preceding embodiments, comprising a W substitution at a position corresponding to A117 SEQ ID NO: 1.

43. The KOD polymerase variant of any one of the preceding embodiments, comprising an A, C, D, E, F, G, H, I, K, L, M, N, P, Q, S, T, V, W, or Y substitution at a position corresponding to R119 of SEQ ID NO: 1.

43b. The KOD polymerase variant of any one of the preceding embodiments, comprising an A substitution at a position corresponding to R119 SEQ ID NO: 1.

43c. The KOD polymerase variant of any one of the preceding embodiments, comprising a C substitution at a position corresponding to R119 SEQ ID NO: 1.

43d. The KOD polymerase variant of any one of the preceding embodiments, comprising a D substitution at a position corresponding to R119 SEQ ID NO: 1.

43e. The KOD polymerase variant of any one of the preceding embodiments, comprising an E substitution at a position corresponding to R119 SEQ ID NO: 1.

43f. The KOD polymerase variant of any one of the preceding embodiments, comprising a F substitution at a position corresponding to R119 SEQ ID NO: 1.

43g. The KOD polymerase variant of any one of the preceding embodiments, comprising a G substitution at a position corresponding to R119 SEQ ID NO: 1.

43h. The KOD polymerase variant of any one of the preceding embodiments, comprising a H substitution at a position corresponding to R119 SEQ ID NO: 1.

43i. The KOD polymerase variant of any one of the preceding embodiments, comprising an I substitution at a position corresponding to R119 SEQ ID NO: 1.

43j. The KOD polymerase variant of any one of the preceding embodiments, comprising a K substitution at a position corresponding to R119 SEQ ID NO: 1.

43k. The KOD polymerase variant of any one of the preceding embodiments, comprising a L substitution at a position corresponding to R119 SEQ ID NO: 1.

43l. The KOD polymerase variant of any one of the preceding embodiments, comprising a M substitution at a position corresponding to R119 SEQ ID NO: 1.

43m. The KOD polymerase variant of any one of the preceding embodiments, comprising a N substitution at a position corresponding to R119 SEQ ID NO: 1.

43n. The KOD polymerase variant of any one of the preceding embodiments, comprising a P substitution at a position corresponding to R119 SEQ ID NO: 1.

43o. The KOD polymerase variant of any one of the preceding embodiments, comprising a Q substitution at a position corresponding to R119 SEQ ID NO: 1.

43p. The KOD polymerase variant of any one of the preceding embodiments, comprising a S substitution at a position corresponding to R119 SEQ ID NO: 1.

43q. The KOD polymerase variant of any one of the preceding embodiments, comprising a T substitution at a position corresponding to R119 SEQ ID NO: 1.

43r. The KOD polymerase variant of any one of the preceding embodiments, comprising a V substitution at a position corresponding to R119 SEQ ID NO: 1.

43s. The KOD polymerase variant of any one of the preceding embodiments, comprising a W substitution at a position corresponding to R119 SEQ ID NO: 1.

43t. The KOD polymerase variant of any one of the preceding embodiments, comprising a Y substitution at a position corresponding to R119 SEQ ID NO: 1.

44. The KOD polymerase variant of any one of the preceding embodiments, comprising a P substitution at a position corresponding to Y120 of SEQ ID NO: 1.

45. The KOD polymerase variant of any one of the preceding embodiments, comprising a K, R, or W substitution at a position corresponding to I122 of SEQ ID NO: 1.

45b. The KOD polymerase variant of any one of the preceding embodiments, comprising a K substitution at a position corresponding to I122 SEQ ID NO: 1.

45c. The KOD polymerase variant of any one of the preceding embodiments, comprising a R substitution at a position corresponding to I122 SEQ ID NO: 1.

45d. The KOD polymerase variant of any one of the preceding embodiments, comprising a W substitution at a position corresponding to I122 SEQ ID NO: 1.

46. The KOD polymerase variant of any one of the preceding embodiments, comprising an A, C, F, H, I, K, L, M, Q, R, S, T, V, W, or a Y substitution at a position corresponding to D123 of SEQ ID NO: 1.

46b. The KOD polymerase variant of any one of the preceding embodiments, comprising an A substitution at a position corresponding to D123 SEQ ID NO: 1.

46c. The KOD polymerase variant of any one of the preceding embodiments, comprising a C substitution at a position corresponding to D123 SEQ ID NO: 1.

46d. The KOD polymerase variant of any one of the preceding embodiments, comprising a F substitution at a position corresponding to D123 SEQ ID NO: 1.

46e. The KOD polymerase variant of any one of the preceding embodiments, comprising a H substitution at a position corresponding to D123 SEQ ID NO: 1.

46f. The KOD polymerase variant of any one of the preceding embodiments, comprising an I substitution at a position corresponding to D123 SEQ ID NO: 1.

46g. The KOD polymerase variant of any one of the preceding embodiments, comprising a K substitution at a position corresponding to D123 SEQ ID NO: 1.

46h. The KOD polymerase variant of any one of the preceding embodiments, comprising a L substitution at a position corresponding to D123 SEQ ID NO: 1.

46i. The KOD polymerase variant of any one of the preceding embodiments, comprising a M substitution at a position corresponding to D123 SEQ ID NO: 1.

46j. The KOD polymerase variant of any one of the preceding embodiments, comprising a Q substitution at a position corresponding to D123 SEQ ID NO: 1.

46k. The KOD polymerase variant of any one of the preceding embodiments, comprising a R substitution at a position corresponding to D123 SEQ ID NO: 1.

46l. The KOD polymerase variant of any one of the preceding embodiments, comprising a S substitution at a position corresponding to D123 SEQ ID NO: 1.

46m. The KOD polymerase variant of any one of the preceding embodiments, comprising a T substitution at a position corresponding to D123 SEQ ID NO: 1.

46n. The KOD polymerase variant of any one of the preceding embodiments, comprising a V substitution at a position corresponding to D123 SEQ ID NO: 1.

46o. The KOD polymerase variant of any one of the preceding embodiments, comprising a W substitution at a position corresponding to D123 SEQ ID NO: 1.

46p. The KOD polymerase variant of any one of the preceding embodiments, comprising a Y substitution at a position corresponding to D123 SEQ ID NO: 1.

47. The KOD polymerase variant of any one of the preceding embodiments, comprising a N substitution at a position corresponding to L138 of SEQ ID NO: 1.

48. The KOD polymerase variant of any one of the preceding embodiments, comprising an E or Q substitution at a position corresponding to A139 of SEQ ID NO: 1.

48b. The KOD polymerase variant of any one of the preceding embodiments, comprising an E substitution at a position corresponding to A139 SEQ ID NO: 1.

48c. The KOD polymerase variant of any one of the preceding embodiments, comprising a Q substitution at a position corresponding to A139 SEQ ID NO: 1.

49. The KOD polymerase variant of any one of the preceding embodiments, comprising a R substitution at a position corresponding to F140 of SEQ ID NO: 1.

50. The KOD polymerase variant of any one of the preceding embodiments, comprising an A, C, G, H, K, N, P, Q, S, T, or V substitution at a position corresponding to D141 of SEQ ID NO: 1.

50b. The KOD polymerase variant of any one of the preceding embodiments, comprising an A substitution at a position corresponding to D141 SEQ ID NO: 1.

50c. The KOD polymerase variant of any one of the preceding embodiments, comprising a C substitution at a position corresponding to D141 SEQ ID NO: 1.

50d. The KOD polymerase variant of any one of the preceding embodiments, comprising a G substitution at a position corresponding to D141 SEQ ID NO: 1.

50e. The KOD polymerase variant of any one of the preceding embodiments, comprising a H substitution at a position corresponding to D141 SEQ ID NO: 1.

50f. The KOD polymerase variant of any one of the preceding embodiments, comprising a K substitution at a position corresponding to D141 SEQ ID NO: 1.

50g. The KOD polymerase variant of any one of the preceding embodiments, comprising a N substitution at a position corresponding to D141 SEQ ID NO: 1.

50h. The KOD polymerase variant of any one of the preceding embodiments, comprising a P substitution at a position corresponding to D141 SEQ ID NO: 1.

50i. The KOD polymerase variant of any one of the preceding embodiments, comprising a Q substitution at a position corresponding to D141 SEQ ID NO: 1.

50j. The KOD polymerase variant of any one of the preceding embodiments, comprising a S substitution at a position corresponding to D141 SEQ ID NO: 1.

50k. The KOD polymerase variant of any one of the preceding embodiments, comprising a T substitution at a position corresponding to D141 SEQ ID NO: 1.

50l. The KOD polymerase variant of any one of the preceding embodiments, comprising a V substitution at a position corresponding to D141 SEQ ID NO: 1.

51. The KOD polymerase variant of any one of the preceding embodiments, comprising a F, G, H, K, P, R, W, or Y substitution at a position corresponding to I142 of SEQ ID NO: 1.

51b. The KOD polymerase variant of any one of the preceding embodiments, comprising a F substitution at a position corresponding to I142 SEQ ID NO: 1.

51c. The KOD polymerase variant of any one of the preceding embodiments, comprising a G substitution at a position corresponding to I142 SEQ ID NO: 1.

51d. The KOD polymerase variant of any one of the preceding embodiments, comprising a H substitution at a position corresponding to I142 SEQ ID NO: 1.

51e. The KOD polymerase variant of any one of the preceding embodiments, comprising a K substitution at a position corresponding to I142 SEQ ID NO: 1.

51f. The KOD polymerase variant of any one of the preceding embodiments, comprising a P substitution at a position corresponding to I142 SEQ ID NO: 1.

51g. The KOD polymerase variant of any one of the preceding embodiments, comprising a R substitution at a position corresponding to I142 SEQ ID NO: 1.

51h. The KOD polymerase variant of any one of the preceding embodiments, comprising a W substitution at a position corresponding to I142 SEQ ID NO: 1.

51i. The KOD polymerase variant of any one of the preceding embodiments, comprising a Y substitution at a position corresponding to I142 SEQ ID NO: 1.

52. The KOD polymerase variant of any one of the preceding embodiments, comprising an A, C, F, G, H, I, K, L, M, N, P, Q, R, S, T, V, W, or Y substitution at a position corresponding to E143 of SEQ ID NO: 1.

52b. The KOD polymerase variant of any one of the preceding embodiments, comprising an A substitution at a position corresponding to E143 SEQ ID NO: 1.

52c. The KOD polymerase variant of any one of the preceding embodiments, comprising a C substitution at a position corresponding to E143 SEQ ID NO: 1.

52d. The KOD polymerase variant of any one of the preceding embodiments, comprising a F substitution at a position corresponding to E143 SEQ ID NO: 1.

52e. The KOD polymerase variant of any one of the preceding embodiments, comprising a G substitution at a position corresponding to E143 SEQ ID NO: 1.

52f. The KOD polymerase variant of any one of the preceding embodiments, comprising a H substitution at a position corresponding to E143 SEQ ID NO: 1.

52g. The KOD polymerase variant of any one of the preceding embodiments, comprising an I substitution at a position corresponding to E143 SEQ ID NO: 1.

52h. The KOD polymerase variant of any one of the preceding embodiments, comprising a K substitution at a position corresponding to E143 SEQ ID NO: 1.

52i. The KOD polymerase variant of any one of the preceding embodiments, comprising a L substitution at a position corresponding to E143 SEQ ID NO: 1.

52j. The KOD polymerase variant of any one of the preceding embodiments, comprising a M substitution at a position corresponding to E143 SEQ ID NO: 1.

52k. The KOD polymerase variant of any one of the preceding embodiments, comprising a N substitution at a position corresponding to E143 SEQ ID NO: 1.

52l. The KOD polymerase variant of any one of the preceding embodiments, comprising a P substitution at a position corresponding to E143 SEQ ID NO: 1.

52m. The KOD polymerase variant of any one of the preceding embodiments, comprising a Q substitution at a position corresponding to E143 SEQ ID NO: 1.

52n. The KOD polymerase variant of any one of the preceding embodiments, comprising a R substitution at a position corresponding to E143 SEQ ID NO: 1.

52o. The KOD polymerase variant of any one of the preceding embodiments, comprising a S substitution at a position corresponding to E143 SEQ ID NO: 1.

52p. The KOD polymerase variant of any one of the preceding embodiments, comprising a T substitution at a position corresponding to E143 SEQ ID NO: 1.

52q. The KOD polymerase variant of any one of the preceding embodiments, comprising a V substitution at a position corresponding to E143 SEQ ID NO: 1.

52r. The KOD polymerase variant of any one of the preceding embodiments, comprising a W substitution at a position corresponding to E143 SEQ ID NO: 1.

52s. The KOD polymerase variant of any one of the preceding embodiments, comprising a Y substitution at a position corresponding to E143 SEQ ID NO: 1.

53. The KOD polymerase variant of any one of the preceding embodiments, comprising an A, D, E, F, G, H, K, L, M, P, Q, R, W, or Y substitution at a position corresponding to T144 of SEQ ID NO: 1.

53b. The KOD polymerase variant of any one of the preceding embodiments, comprising an A substitution at a position corresponding to T144 SEQ ID NO: 1.

53c. The KOD polymerase variant of any one of the preceding embodiments, comprising a D substitution at a position corresponding to T144 SEQ ID NO: 1.

53d. The KOD polymerase variant of any one of the preceding embodiments, comprising an E substitution at a position corresponding to T144 SEQ ID NO: 1.

53e. The KOD polymerase variant of any one of the preceding embodiments, comprising a F substitution at a position corresponding to T144 SEQ ID NO: 1.

53f. The KOD polymerase variant of any one of the preceding embodiments, comprising a G substitution at a position corresponding to T144 SEQ ID NO: 1.

53g. The KOD polymerase variant of any one of the preceding embodiments, comprising a H substitution at a position corresponding to T144 SEQ ID NO: 1.

53h. The KOD polymerase variant of any one of the preceding embodiments, comprising a K substitution at a position corresponding to T144 SEQ ID NO: 1.

53i. The KOD polymerase variant of any one of the preceding embodiments, comprising a L substitution at a position corresponding to T144 SEQ ID NO: 1.

53j. The KOD polymerase variant of any one of the preceding embodiments, comprising a M substitution at a position corresponding to T144 SEQ ID NO: 1.

53k. The KOD polymerase variant of any one of the preceding embodiments, comprising a P substitution at a position corresponding to T144 SEQ ID NO: 1.

53l. The KOD polymerase variant of any one of the preceding embodiments, comprising a Q substitution at a position corresponding to T144 SEQ ID NO: 1.

53m. The KOD polymerase variant of any one of the preceding embodiments, comprising a R substitution at a position corresponding to T144 SEQ ID NO: 1.

53n. The KOD polymerase variant of any one of the preceding embodiments, comprising a W substitution at a position corresponding to T144 SEQ ID NO: 1.

53o. The KOD polymerase variant of any one of the preceding embodiments, comprising a Y substitution at a position corresponding to T144 SEQ ID NO: 1.

54. The KOD polymerase variant of any one of the preceding embodiments, comprising a G substitution at a position corresponding to L145 of SEQ ID NO: 1.

55. The KOD polymerase variant of any one of the preceding embodiments, comprising a D or E substitution at a position corresponding to Y146 of SEQ ID NO: 1.

55b. The KOD polymerase variant of any one of the preceding embodiments, comprising a D substitution at a position corresponding to Y146 SEQ ID NO: 1.

55c. The KOD polymerase variant of any one of the preceding embodiments, comprising an E substitution at a position corresponding to Y146 SEQ ID NO: 1.

56. The KOD polymerase variant of any one of the preceding embodiments, comprising a L substitution at a position corresponding to G149 of SEQ ID NO: 1.

57. The KOD polymerase variant of any one of the preceding embodiments, comprising an A, D, E, N, or S substitution at a position corresponding to F152 of SEQ ID NO: 1.

57b. The KOD polymerase variant of any one of the preceding embodiments, comprising an A substitution at a position corresponding to F152 SEQ ID NO: 1.

57c. The KOD polymerase variant of any one of the preceding embodiments, comprising a D substitution at a position corresponding to F152 SEQ ID NO: 1.

57d. The KOD polymerase variant of any one of the preceding embodiments, comprising an E substitution at a position corresponding to F152 SEQ ID NO: 1.

57e. The KOD polymerase variant of any one of the preceding embodiments, comprising a N substitution at a position corresponding to F152 SEQ ID NO: 1.

57f. The KOD polymerase variant of any one of the preceding embodiments, comprising a S substitution at a position corresponding to F152 SEQ ID NO: 1.

58. The KOD polymerase variant of any one of the preceding embodiments, comprising a P or Y substitution at a position corresponding to E154 of SEQ ID NO: 1.

58b. The KOD polymerase variant of any one of the preceding embodiments, comprising a P substitution at a position corresponding to E154 SEQ ID NO: 1.

58c. The KOD polymerase variant of any one of the preceding embodiments, comprising a Y substitution at a position corresponding to E154 SEQ ID NO: 1.

59. The KOD polymerase variant of any one of the preceding embodiments, comprising an I or V substitution at a position corresponding to G155 of SEQ ID NO: 1.

59b. The KOD polymerase variant of any one of the preceding embodiments, comprising an I substitution at a position corresponding to G155 SEQ ID NO: 1.

59c. The KOD polymerase variant of any one of the preceding embodiments, comprising a V substitution at a position corresponding to G155 SEQ ID NO: 1.

60. The KOD polymerase variant of any one of the preceding embodiments, comprising an E, F, R, W, or Y substitution at a position corresponding to I157 of SEQ ID NO: 1.

60b. The KOD polymerase variant of any one of the preceding embodiments, comprising an E substitution at a position corresponding to I157 SEQ ID NO: 1.

60c. The KOD polymerase variant of any one of the preceding embodiments, comprising a F substitution at a position corresponding to I157 SEQ ID NO: 1.

60d. The KOD polymerase variant of any one of the preceding embodiments, comprising a R substitution at a position corresponding to I157 SEQ ID NO: 1.

60e. The KOD polymerase variant of any one of the preceding embodiments, comprising a W substitution at a position corresponding to I157 SEQ ID NO: 1.

60f. The KOD polymerase variant of any one of the preceding embodiments, comprising a Y substitution at a position corresponding to I157 SEQ ID NO: 1.

61. The KOD polymerase variant of any one of the preceding embodiments, comprising a D, H, K, Q, R, W, or Y substitution at a position corresponding to L158 of SEQ ID NO: 1.

61b. The KOD polymerase variant of any one of the preceding embodiments, comprising a D substitution at a position corresponding to L158 SEQ ID NO: 1.

61c. The KOD polymerase variant of any one of the preceding embodiments, comprising a H substitution at a position corresponding to L158 SEQ ID NO: 1.

61d. The KOD polymerase variant of any one of the preceding embodiments, comprising a K substitution at a position corresponding to L158 SEQ ID NO: 1.

61e. The KOD polymerase variant of any one of the preceding embodiments, comprising a Q substitution at a position corresponding to L158 SEQ ID NO: 1.

61f. The KOD polymerase variant of any one of the preceding embodiments, comprising a R substitution at a position corresponding to L158 SEQ ID NO: 1.

61g. The KOD polymerase variant of any one of the preceding embodiments, comprising a W substitution at a position corresponding to L158 SEQ ID NO: 1.

61h. The KOD polymerase variant of any one of the preceding embodiments, comprising a Y substitution at a position corresponding to L158 SEQ ID NO: 1.

62. The KOD polymerase variant of any one of the preceding embodiments, comprising an E, G, P, or R substitution at a position corresponding to M159 of SEQ ID NO: 1.

62b. The KOD polymerase variant of any one of the preceding embodiments, comprising an E substitution at a position corresponding to M159 SEQ ID NO: 1.

62c. The KOD polymerase variant of any one of the preceding embodiments, comprising a G substitution at a position corresponding to M159 SEQ ID NO: 1.

62d. The KOD polymerase variant of any one of the preceding embodiments, comprising a P substitution at a position corresponding to M159 SEQ ID NO: 1.

62e. The KOD polymerase variant of any one of the preceding embodiments, comprising a R substitution at a position corresponding to M159 SEQ ID NO: 1.

63. The KOD polymerase variant of any one of the preceding embodiments, comprising a F substitution at a position corresponding to I160 of SEQ ID NO: 1.

64. The KOD polymerase variant of any one of the preceding embodiments, comprising a D, E, F, H, K, L, M, N, P, R, T, W, or Y substitution at a position corresponding to S161 of SEQ ID NO: 1.

64b. The KOD polymerase variant of any one of the preceding embodiments, comprising a D substitution at a position corresponding to S161 SEQ ID NO: 1.

64c. The KOD polymerase variant of any one of the preceding embodiments, comprising an E substitution at a position corresponding to S161 SEQ ID NO: 1.

64d. The KOD polymerase variant of any one of the preceding embodiments, comprising a F substitution at a position corresponding to S161 SEQ ID NO: 1.

64e. The KOD polymerase variant of any one of the preceding embodiments, comprising a H substitution at a position corresponding to S161 SEQ ID NO: 1.

64f. The KOD polymerase variant of any one of the preceding embodiments, comprising a K substitution at a position corresponding to S161 SEQ ID NO: 1.

64g. The KOD polymerase variant of any one of the preceding embodiments, comprising a L substitution at a position corresponding to S161 SEQ ID NO: 1.

64h. The KOD polymerase variant of any one of the preceding embodiments, comprising a M substitution at a position corresponding to S161 SEQ ID NO: 1.

64i. The KOD polymerase variant of any one of the preceding embodiments, comprising a N substitution at a position corresponding to S161 SEQ ID NO: 1.

64j. The KOD polymerase variant of any one of the preceding embodiments, comprising a P substitution at a position corresponding to S161 SEQ ID NO: 1.

64k. The KOD polymerase variant of any one of the preceding embodiments, comprising a R substitution at a position corresponding to S161 SEQ ID NO: 1.

64l. The KOD polymerase variant of any one of the preceding embodiments, comprising a T substitution at a position corresponding to S161 SEQ ID NO: 1.

64m. The KOD polymerase variant of any one of the preceding embodiments, comprising a W substitution at a position corresponding to S161 SEQ ID NO: 1.

64n. The KOD polymerase variant of any one of the preceding embodiments, comprising a Y substitution at a position corresponding to S161 SEQ ID NO: 1.

65. The KOD polymerase variant of any one of the preceding embodiments, comprising a P substitution at a position corresponding to Y162 of SEQ ID NO: 1.

66. The KOD polymerase variant of any one of the preceding embodiments, comprising a C, E, F, G, N, or T substitution at a position corresponding to A168 of SEQ ID NO: 1.

66b. The KOD polymerase variant of any one of the preceding embodiments, comprising a C substitution at a position corresponding to A168 SEQ ID NO: 1.

66c. The KOD polymerase variant of any one of the preceding embodiments, comprising an E substitution at a position corresponding to A168 SEQ ID NO: 1.

66d. The KOD polymerase variant of any one of the preceding embodiments, comprising a F substitution at a position corresponding to A168 SEQ ID NO: 1.

66e. The KOD polymerase variant of any one of the preceding embodiments, comprising a G substitution at a position corresponding to A168 SEQ ID NO: 1.

66f. The KOD polymerase variant of any one of the preceding embodiments, comprising a N substitution at a position corresponding to A168 SEQ ID NO: 1.

66g. The KOD polymerase variant of any one of the preceding embodiments, comprising a T substitution at a position corresponding to A168 SEQ ID NO: 1.

67. The KOD polymerase variant of any one of the preceding embodiments, comprising an A, G, H, R, S, W, or Y substitution at a position corresponding to V170 of SEQ ID NO: 1.

67b. The KOD polymerase variant of any one of the preceding embodiments, comprising an A substitution at a position corresponding to V170 SEQ ID NO: 1.

67c. The KOD polymerase variant of any one of the preceding embodiments, comprising a G substitution at a position corresponding to V170 SEQ ID NO: 1.

67d. The KOD polymerase variant of any one of the preceding embodiments, comprising a H substitution at a position corresponding to V170 SEQ ID NO: 1.

67e. The KOD polymerase variant of any one of the preceding embodiments, comprising a R substitution at a position corresponding to V170 SEQ ID NO: 1.

67f. The KOD polymerase variant of any one of the preceding embodiments, comprising a S substitution at a position corresponding to V170 SEQ ID NO: 1.

67g. The KOD polymerase variant of any one of the preceding embodiments, comprising a W substitution at a position corresponding to V170 SEQ ID NO: 1.

67h. The KOD polymerase variant of any one of the preceding embodiments, comprising a Y substitution at a position corresponding to V170 SEQ ID NO: 1.

68. The KOD polymerase variant of any one of the preceding embodiments, comprising a R substitution at a position corresponding to T172 of SEQ ID NO: 1.

69. The KOD polymerase variant of any one of the preceding embodiments, comprising a W substitution at a position corresponding to E187 of SEQ ID NO: 1.

70. The KOD polymerase variant of any one of the preceding embodiments, comprising a F or Y substitution at a position corresponding to I191 of SEQ ID NO: 1.

70b. The KOD polymerase variant of any one of the preceding embodiments, comprising a F substitution at a position corresponding to I191 SEQ ID NO: 1.

70c. The KOD polymerase variant of any one of the preceding embodiments, comprising a Y substitution at a position corresponding to I191 SEQ ID NO: 1.

71. The KOD polymerase variant of any one of the preceding embodiments, comprising a G substitution at a position corresponding to F194 of SEQ ID NO: 1.

72. The KOD polymerase variant of any one of the preceding embodiments, comprising a F substitution at a position corresponding to K199 of SEQ ID NO: 1.

73. The KOD polymerase variant of any one of the preceding embodiments, comprising an E, F, H, I, K, N, Q, R, V, W, or Y substitution at a position corresponding to T208 of SEQ ID NO: 1.

73b. The KOD polymerase variant of any one of the preceding embodiments, comprising an E substitution at a position corresponding to T208 SEQ ID NO: 1.

73c. The KOD polymerase variant of any one of the preceding embodiments, comprising a F substitution at a position corresponding to T208 SEQ ID NO: 1.

73d. The KOD polymerase variant of any one of the preceding embodiments, comprising a H substitution at a position corresponding to T208 SEQ ID NO: 1.

73e. The KOD polymerase variant of any one of the preceding embodiments, comprising an I substitution at a position corresponding to T208 SEQ ID NO: 1.

73f. The KOD polymerase variant of any one of the preceding embodiments, comprising a K substitution at a position corresponding to T208 SEQ ID NO: 1.

73g. The KOD polymerase variant of any one of the preceding embodiments, comprising a N substitution at a position corresponding to T208 SEQ ID NO: 1.

73h. The KOD polymerase variant of any one of the preceding embodiments, comprising a Q substitution at a position corresponding to T208 SEQ ID NO: 1.

73i. The KOD polymerase variant of any one of the preceding embodiments, comprising a R substitution at a position corresponding to T208 SEQ ID NO: 1.

73j. The KOD polymerase variant of any one of the preceding embodiments, comprising a V substitution at a position corresponding to T208 SEQ ID NO: 1.

73k. The KOD polymerase variant of any one of the preceding embodiments, comprising a W substitution at a position corresponding to T208 SEQ ID NO: 1.

73l. The KOD polymerase variant of any one of the preceding embodiments, comprising a Y substitution at a position corresponding to T208 SEQ ID NO: 1.

74. The KOD polymerase variant of any one of the preceding embodiments, comprising an A, C, E, G, H, I, L, M, N, P, S, T, V, or W substitution at a position corresponding to Y209 of SEQ ID NO: 1.

74b. The KOD polymerase variant of any one of the preceding embodiments, comprising an A substitution at a position corresponding to Y209 SEQ ID NO: 1.

74c. The KOD polymerase variant of any one of the preceding embodiments, comprising a C substitution at a position corresponding to Y209 SEQ ID NO: 1.

74d. The KOD polymerase variant of any one of the preceding embodiments, comprising an E substitution at a position corresponding to Y209 SEQ ID NO: 1.

74e. The KOD polymerase variant of any one of the preceding embodiments, comprising a G substitution at a position corresponding to Y209 SEQ ID NO: 1.

74f. The KOD polymerase variant of any one of the preceding embodiments, comprising a H substitution at a position corresponding to Y209 SEQ ID NO: 1.

74g. The KOD polymerase variant of any one of the preceding embodiments, comprising an I substitution at a position corresponding to Y209 SEQ ID NO: 1.

74h. The KOD polymerase variant of any one of the preceding embodiments, comprising a L substitution at a position corresponding to Y209 SEQ ID NO: 1.

74i. The KOD polymerase variant of any one of the preceding embodiments, comprising a M substitution at a position corresponding to Y209 SEQ ID NO: 1.

74j. The KOD polymerase variant of any one of the preceding embodiments, comprising a N substitution at a position corresponding to Y209 SEQ ID NO: 1.

74k. The KOD polymerase variant of any one of the preceding embodiments, comprising a P substitution at a position corresponding to Y209 SEQ ID NO: 1.

74l. The KOD polymerase variant of any one of the preceding embodiments, comprising a S substitution at a position corresponding to Y209 SEQ ID NO: 1.

74m. The KOD polymerase variant of any one of the preceding embodiments, comprising a T substitution at a position corresponding to Y209 SEQ ID NO: 1.

74n. The KOD polymerase variant of any one of the preceding embodiments, comprising a V substitution at a position corresponding to Y209 SEQ ID NO: 1.

74o. The KOD polymerase variant of any one of the preceding embodiments, comprising a W substitution at a position corresponding to Y209 SEQ ID NO: 1.

75. The KOD polymerase variant of any one of the preceding embodiments, comprising a C, D, E, G, M, P, Q, S, or T substitution at a position corresponding to N210 of SEQ ID NO: 1.

75b. The KOD polymerase variant of any one of the preceding embodiments, comprising a C substitution at a position corresponding to N210 SEQ ID NO: 1.

75c. The KOD polymerase variant of any one of the preceding embodiments, comprising a D substitution at a position corresponding to N210 SEQ ID NO: 1.

75d. The KOD polymerase variant of any one of the preceding embodiments, comprising an E substitution at a position corresponding to N210 SEQ ID NO: 1.

75e. The KOD polymerase variant of any one of the preceding embodiments, comprising a G substitution at a position corresponding to N210 SEQ ID NO: 1.

75f. The KOD polymerase variant of any one of the preceding embodiments, comprising a M substitution at a position corresponding to N210 SEQ ID NO: 1.

75g. The KOD polymerase variant of any one of the preceding embodiments, comprising a P substitution at a position corresponding to N210 SEQ ID NO: 1.

75h. The KOD polymerase variant of any one of the preceding embodiments, comprising a Q substitution at a position corresponding to N210 SEQ ID NO: 1.

75i. The KOD polymerase variant of any one of the preceding embodiments, comprising a S substitution at a position corresponding to N210 SEQ ID NO: 1.

75j. The KOD polymerase variant of any one of the preceding embodiments, comprising a T substitution at a position corresponding to N210 SEQ ID NO: 1.

76. The KOD polymerase variant of any one of the preceding embodiments, comprising a C, D, E, F, H, I, K, L, M, N, P, Q, R, S, T, V, W or Y substitution at a position corresponding to G211 of SEQ ID NO: 1.

76b. The KOD polymerase variant of any one of the preceding embodiments, comprising an C substitution at a position corresponding to G211 SEQ ID NO: 1.

76c. The KOD polymerase variant of any one of the preceding embodiments, comprising a D substitution at a position corresponding to G211 SEQ ID NO: 1.

76d. The KOD polymerase variant of any one of the preceding embodiments, comprising an E substitution at a position corresponding to G211 SEQ ID NO: 1.

76e. The KOD polymerase variant of any one of the preceding embodiments, comprising a F substitution at a position corresponding to G211 SEQ ID NO: 1.

76f. The KOD polymerase variant of any one of the preceding embodiments, comprising a H substitution at a position corresponding to G211 SEQ ID NO: 1.

76g. The KOD polymerase variant of any one of the preceding embodiments, comprising an I substitution at a position corresponding to G211 SEQ ID NO: 1.

76h. The KOD polymerase variant of any one of the preceding embodiments, comprising a K substitution at a position corresponding to G211 SEQ ID NO: 1.

76i. The KOD polymerase variant of any one of the preceding embodiments, comprising a L substitution at a position corresponding to G211 SEQ ID NO: 1.

76j. The KOD polymerase variant of any one of the preceding embodiments, comprising a M substitution at a position corresponding to G211 SEQ ID NO: 1.

76k. The KOD polymerase variant of any one of the preceding embodiments, comprising a N substitution at a position corresponding to G211 SEQ ID NO: 1.

76l. The KOD polymerase variant of any one of the preceding embodiments, comprising a P substitution at a position corresponding to G211 SEQ ID NO: 1.

76m. The KOD polymerase variant of any one of the preceding embodiments, comprising a Q substitution at a position corresponding to G211 SEQ ID NO: 1.

76n. The KOD polymerase variant of any one of the preceding embodiments, comprising a R substitution at a position corresponding to G211 SEQ ID NO: 1.

76o. The KOD polymerase variant of any one of the preceding embodiments, comprising a S substitution at a position corresponding to G211 SEQ ID NO: 1.

76p. The KOD polymerase variant of any one of the preceding embodiments, comprising a T substitution at a position corresponding to G211 SEQ ID NO: 1.

76q. The KOD polymerase variant of any one of the preceding embodiments, comprising a V substitution at a position corresponding to G211 SEQ ID NO: 1.

76r. The KOD polymerase variant of any one of the preceding embodiments, comprising a W substitution at a position corresponding to G211 SEQ ID NO: 1.

76s. The KOD polymerase variant of any one of the preceding embodiments, comprising a Y substitution at a position corresponding to G211 SEQ ID NO: 1.

77. The KOD polymerase variant of any one of the preceding embodiments, comprising a F, I, K, L, M, P, Q, R, V, W, or Y substitution at a position corresponding to D212 of SEQ ID NO: 1.

77b. The KOD polymerase variant of any one of the preceding embodiments, comprising a F substitution at a position corresponding to D212 SEQ ID NO: 1.

77c. The KOD polymerase variant of any one of the preceding embodiments, comprising an I substitution at a position corresponding to D212 SEQ ID NO: 1.

77d. The KOD polymerase variant of any one of the preceding embodiments, comprising a K substitution at a position corresponding to D212 SEQ ID NO: 1.

77e. The KOD polymerase variant of any one of the preceding embodiments, comprising a L substitution at a position corresponding to D212 SEQ ID NO: 1.

77f. The KOD polymerase variant of any one of the preceding embodiments, comprising a M substitution at a position corresponding to D212 SEQ ID NO: 1.

77g. The KOD polymerase variant of any one of the preceding embodiments, comprising a P substitution at a position corresponding to D212 SEQ ID NO: 1.

77h. The KOD polymerase variant of any one of the preceding embodiments, comprising a Q substitution at a position corresponding to D212 SEQ ID NO: 1.

77i. The KOD polymerase variant of any one of the preceding embodiments, comprising a R substitution at a position corresponding to D212 SEQ ID NO: 1.

77j. The KOD polymerase variant of any one of the preceding embodiments, comprising a V substitution at a position corresponding to D212 SEQ ID NO: 1.

77k. The KOD polymerase variant of any one of the preceding embodiments, comprising a W substitution at a position corresponding to D212 SEQ ID NO: 1.

77l. The KOD polymerase variant of any one of the preceding embodiments, comprising a Y substitution at a position corresponding to D212 SEQ ID NO: 1.

78. The KOD polymerase variant of any one of the preceding embodiments, comprising a D or E substitution at a position corresponding to N213 of SEQ ID NO: 1.

78b. The KOD polymerase variant of any one of the preceding embodiments, comprising a D substitution at a position corresponding to N213 SEQ ID NO: 1.

78c. The KOD polymerase variant of any one of the preceding embodiments, comprising an E substitution at a position corresponding to N213 SEQ ID NO: 1.

79. The KOD polymerase variant of any one of the preceding embodiments, comprising an A, C, D, E, G, H, I, K, L, M, N, P, Q, R, S, T, V, or W substitution at a position corresponding to F214 of SEQ ID NO: 1.

79b. The KOD polymerase variant of any one of the preceding embodiments, comprising an A substitution at a position corresponding to F214 SEQ ID NO: 1.

79c. The KOD polymerase variant of any one of the preceding embodiments, comprising a C substitution at a position corresponding to F214 SEQ ID NO: 1.

79d. The KOD polymerase variant of any one of the preceding embodiments, comprising a D substitution at a position corresponding to F214 SEQ ID NO: 1.

79e. The KOD polymerase variant of any one of the preceding embodiments, comprising an E substitution at a position corresponding to F214 SEQ ID NO: 1.

79f. The KOD polymerase variant of any one of the preceding embodiments, comprising a G substitution at a position corresponding to F214 SEQ ID NO: 1.

79g. The KOD polymerase variant of any one of the preceding embodiments, comprising a H substitution at a position corresponding to F214 SEQ ID NO: 1.

79h. The KOD polymerase variant of any one of the preceding embodiments, comprising an I substitution at a position corresponding to F214 SEQ ID NO: 1.

79i. The KOD polymerase variant of any one of the preceding embodiments, comprising a K substitution at a position corresponding to F214 SEQ ID NO: 1.

79j. The KOD polymerase variant of any one of the preceding embodiments, comprising a L substitution at a position corresponding to F214 SEQ ID NO: 1.

79k. The KOD polymerase variant of any one of the preceding embodiments, comprising a M substitution at a position corresponding to F214 SEQ ID NO: 1.

79l. The KOD polymerase variant of any one of the preceding embodiments, comprising a N substitution at a position corresponding to F214 SEQ ID NO: 1.

79m. The KOD polymerase variant of any one of the preceding embodiments, comprising a P substitution at a position corresponding to F214 SEQ ID NO: 1.

79n. The KOD polymerase variant of any one of the preceding embodiments, comprising a Q substitution at a position corresponding to F214 SEQ ID NO: 1.

79o. The KOD polymerase variant of any one of the preceding embodiments, comprising a R substitution at a position corresponding to F214 SEQ ID NO: 1.

79p. The KOD polymerase variant of any one of the preceding embodiments, comprising a S substitution at a position corresponding to F214 SEQ ID NO: 1.

79q. The KOD polymerase variant of any one of the preceding embodiments, comprising a T substitution at a position corresponding to F214 SEQ ID NO: 1.

79r. The KOD polymerase variant of any one of the preceding embodiments, comprising a V substitution at a position corresponding to F214 SEQ ID NO: 1.

79s. The KOD polymerase variant of any one of the preceding embodiments, comprising a W substitution at a position corresponding to F214 SEQ ID NO: 1.

80. The KOD polymerase variant of any one of the preceding embodiments, comprising an A, C, F, G, H, I, K, L, M, N, P, Q, R, S, T, V, W, or Y substitution at a position corresponding to D215 of SEQ ID NO: 1.

80b. The KOD polymerase variant of any one of the preceding embodiments, comprising an A substitution at a position corresponding to D215 SEQ ID NO: 1.

80c. The KOD polymerase variant of any one of the preceding embodiments, comprising a C substitution at a position corresponding to D215 SEQ ID NO: 1.

80d. The KOD polymerase variant of any one of the preceding embodiments, comprising a F substitution at a position corresponding to D215 SEQ ID NO: 1.

80e. The KOD polymerase variant of any one of the preceding embodiments, comprising a G substitution at a position corresponding to D215 SEQ ID NO: 1.

80f. The KOD polymerase variant of any one of the preceding embodiments, comprising a H substitution at a position corresponding to D215 SEQ ID NO: 1.

80g. The KOD polymerase variant of any one of the preceding embodiments, comprising an I substitution at a position corresponding to D215 SEQ ID NO: 1.

80h. The KOD polymerase variant of any one of the preceding embodiments, comprising a K substitution at a position corresponding to D215 SEQ ID NO: 1.

80i. The KOD polymerase variant of any one of the preceding embodiments, comprising a L substitution at a position corresponding to D215 SEQ ID NO: 1.

80j. The KOD polymerase variant of any one of the preceding embodiments, comprising a M substitution at a position corresponding to D215 SEQ ID NO: 1.

80k. The KOD polymerase variant of any one of the preceding embodiments, comprising a N substitution at a position corresponding to D215 SEQ ID NO: 1.

80l. The KOD polymerase variant of any one of the preceding embodiments, comprising a P substitution at a position corresponding to D215 SEQ ID NO: 1.

80m. The KOD polymerase variant of any one of the preceding embodiments, comprising a Q substitution at a position corresponding to D215 SEQ ID NO: 1.

80n. The KOD polymerase variant of any one of the preceding embodiments, comprising a R substitution at a position corresponding to D215 SEQ ID NO: 1.

80o. The KOD polymerase variant of any one of the preceding embodiments, comprising a S substitution at a position corresponding to D215 SEQ ID NO: 1.

80p. The KOD polymerase variant of any one of the preceding embodiments, comprising a T substitution at a position corresponding to D215 SEQ ID NO: 1.

80q. The KOD polymerase variant of any one of the preceding embodiments, comprising a V substitution at a position corresponding to D215 SEQ ID NO: 1.

80r. The KOD polymerase variant of any one of the preceding embodiments, comprising a W substitution at a position corresponding to D215 SEQ ID NO: 1.

80s. The KOD polymerase variant of any one of the preceding embodiments, comprising a Y substitution at a position corresponding to D215 SEQ ID NO: 1.

81. The KOD polymerase variant of any one of the preceding embodiments, comprising an A or D substitution at a position corresponding to F216 of SEQ ID NO: 1.

81b. The KOD polymerase variant of any one of the preceding embodiments, comprising an A substitution at a position corresponding to F216 SEQ ID NO: 1.

81c. The KOD polymerase variant of any one of the preceding embodiments, comprising a D substitution at a position corresponding to F216 SEQ ID NO: 1.

82. The KOD polymerase variant of any one of the preceding embodiments, comprising an A, D, E, G, L, P, S, T, or W substitution at a position corresponding to Y218 of SEQ ID NO: 1.

82b. The KOD polymerase variant of any one of the preceding embodiments, comprising an A substitution at a position corresponding to Y218 SEQ ID NO: 1.

82c. The KOD polymerase variant of any one of the preceding embodiments, comprising a D substitution at a position corresponding to Y218 SEQ ID NO: 1.

82d. The KOD polymerase variant of any one of the preceding embodiments, comprising an E substitution at a position corresponding to Y218 SEQ ID NO: 1.

82e. The KOD polymerase variant of any one of the preceding embodiments, comprising a G substitution at a position corresponding to Y218 SEQ ID NO: 1.

82f. The KOD polymerase variant of any one of the preceding embodiments, comprising a L substitution at a position corresponding to Y218 SEQ ID NO: 1.

82g. The KOD polymerase variant of any one of the preceding embodiments, comprising a P substitution at a position corresponding to Y218 SEQ ID NO: 1.

82h. The KOD polymerase variant of any one of the preceding embodiments, comprising a S substitution at a position corresponding to Y218 SEQ ID NO: 1.

82i. The KOD polymerase variant of any one of the preceding embodiments, comprising a T substitution at a position corresponding to Y218 SEQ ID NO: 1.

82j. The KOD polymerase variant of any one of the preceding embodiments, comprising a W substitution at a position corresponding to Y218 SEQ ID NO: 1.

83. The KOD polymerase variant of any one of the preceding embodiments, comprising a P, R, or V substitution at a position corresponding to L219 of SEQ ID NO: 1.

83b. The KOD polymerase variant of any one of the preceding embodiments, comprising a P substitution at a position corresponding to L219 SEQ ID NO: 1.

83c. The KOD polymerase variant of any one of the preceding embodiments, comprising a R substitution at a position corresponding to L219 SEQ ID NO: 1.

83d. The KOD polymerase variant of any one of the preceding embodiments, comprising a V substitution at a position corresponding to L219 SEQ ID NO: 1.

84. The KOD polymerase variant of any one of the preceding embodiments, comprising an I or V substitution at a position corresponding to K220 of SEQ ID NO: 1.

84b. The KOD polymerase variant of any one of the preceding embodiments, comprising an I substitution at a position corresponding to K220 SEQ ID NO: 1.

84c. The KOD polymerase variant of any one of the preceding embodiments, comprising a V substitution at a position corresponding to K220 SEQ ID NO: 1.

85. The KOD polymerase variant of any one of the preceding embodiments, comprising an A, E, or P substitution at a position corresponding to C223 of SEQ ID NO: 1.

85b. The KOD polymerase variant of any one of the preceding embodiments, comprising an A substitution at a position corresponding to C223 SEQ ID NO: 1.

85c. The KOD polymerase variant of any one of the preceding embodiments, comprising an E substitution at a position corresponding to C223 SEQ ID NO: 1.

85d. The KOD polymerase variant of any one of the preceding embodiments, comprising a P substitution at a position corresponding to C223 SEQ ID NO: 1.

86. The KOD polymerase variant of any one of the preceding embodiments, comprising a W or Y substitution at a position corresponding to K240 of SEQ ID NO: 1.

86b. The KOD polymerase variant of any one of the preceding embodiments, comprising a W substitution at a position corresponding to K240 SEQ ID NO: 1.

86c. The KOD polymerase variant of any one of the preceding embodiments, comprising a Y substitution at a position corresponding to K240 SEQ ID NO: 1.

87. The KOD polymerase variant of any one of the preceding embodiments, comprising a D substitution at a position corresponding to A249 of SEQ ID NO: 1.

88. The KOD polymerase variant of any one of the preceding embodiments, comprising a L substitution at a position corresponding to V250 of SEQ ID NO: 1.

89. The KOD polymerase variant of any one of the preceding embodiments, comprising a F, G, N, or R substitution at a position corresponding to E251 of SEQ ID NO: 1.

89b. The KOD polymerase variant of any one of the preceding embodiments, comprising a F substitution at a position corresponding to E251 SEQ ID NO: 1.

89c. The KOD polymerase variant of any one of the preceding embodiments, comprising a G substitution at a position corresponding to E251 SEQ ID NO: 1.

89d. The KOD polymerase variant of any one of the preceding embodiments, comprising a N substitution at a position corresponding to E251 SEQ ID NO: 1.

89e. The KOD polymerase variant of any one of the preceding embodiments, comprising a R substitution at a position corresponding to E251 SEQ ID NO: 1.

90. The KOD polymerase variant of any one of the preceding embodiments, comprising a C, E, M, T, or W substitution at a position corresponding to V252 of SEQ ID NO: 1.

90b. The KOD polymerase variant of any one of the preceding embodiments, comprising a C substitution at a position corresponding to V252 SEQ ID NO: 1.

90c. The KOD polymerase variant of any one of the preceding embodiments, comprising an E substitution at a position corresponding to V252 SEQ ID NO: 1.

90d. The KOD polymerase variant of any one of the preceding embodiments, comprising a M substitution at a position corresponding to V252 SEQ ID NO: 1.

90e. The KOD polymerase variant of any one of the preceding embodiments, comprising a T substitution at a position corresponding to V252 SEQ ID NO: 1.

90f. The KOD polymerase variant of any one of the preceding embodiments, comprising a W substitution at a position corresponding to V252 SEQ ID NO: 1.

91. The KOD polymerase variant of any one of the preceding embodiments, comprising a H, N, or R substitution at a position corresponding to G254 of SEQ ID NO: 1.

91b. The KOD polymerase variant of any one of the preceding embodiments, comprising a H substitution at a position corresponding to G254 SEQ ID NO: 1.

91c. The KOD polymerase variant of any one of the preceding embodiments, comprising a N substitution at a position corresponding to G254 SEQ ID NO: 1.

91d. The KOD polymerase variant of any one of the preceding embodiments, comprising a R substitution at a position corresponding to G254 SEQ ID NO: 1.

92. The KOD polymerase variant of any one of the preceding embodiments, comprising a F, W, or Y substitution at a position corresponding to H257 of SEQ ID NO: 1.

92b. The KOD polymerase variant of any one of the preceding embodiments, comprising a F substitution at a position corresponding to H257 SEQ ID NO: 1.

92c. The KOD polymerase variant of any one of the preceding embodiments, comprising a W substitution at a position corresponding to H257 SEQ ID NO: 1.

92d. The KOD polymerase variant of any one of the preceding embodiments, comprising a Y substitution at a position corresponding to H257 SEQ ID NO: 1.

93. The KOD polymerase variant of any one of the preceding embodiments, comprising an A, C, F, G, H, I, K, L, M, N, P, Q, R, S, T, V, W, or Y substitution at a position corresponding to D259 of SEQ ID NO: 1.

93b. The KOD polymerase variant of any one of the preceding embodiments, comprising an A substitution at a position corresponding to D259 SEQ ID NO: 1.

93c. The KOD polymerase variant of any one of the preceding embodiments, comprising a C substitution at a position corresponding to D259 SEQ ID NO: 1.

93d. The KOD polymerase variant of any one of the preceding embodiments, comprising a F substitution at a position corresponding to D259 SEQ ID NO: 1.

93e. The KOD polymerase variant of any one of the preceding embodiments, comprising a G substitution at a position corresponding to D259 SEQ ID NO: 1.

93f. The KOD polymerase variant of any one of the preceding embodiments, comprising a H substitution at a position corresponding to D259 SEQ ID NO: 1.

93g. The KOD polymerase variant of any one of the preceding embodiments, comprising an I substitution at a position corresponding to D259 SEQ ID NO: 1.

93h. The KOD polymerase variant of any one of the preceding embodiments, comprising a K substitution at a position corresponding to D259 SEQ ID NO: 1.

93i. The KOD polymerase variant of any one of the preceding embodiments, comprising a L substitution at a position corresponding to D259 SEQ ID NO: 1.

93j. The KOD polymerase variant of any one of the preceding embodiments, comprising a M substitution at a position corresponding to D259 SEQ ID NO: 1.

93k. The KOD polymerase variant of any one of the preceding embodiments, comprising a N substitution at a position corresponding to D259 SEQ ID NO: 1.

93l. The KOD polymerase variant of any one of the preceding embodiments, comprising a P substitution at a position corresponding to D259 SEQ ID NO: 1.

93m. The KOD polymerase variant of any one of the preceding embodiments, comprising a Q substitution at a position corresponding to D259 SEQ ID NO: 1.

93n. The KOD polymerase variant of any one of the preceding embodiments, comprising a R substitution at a position corresponding to D259 SEQ ID NO: 1.

93o. The KOD polymerase variant of any one of the preceding embodiments, comprising a S substitution at a position corresponding to D259 SEQ ID NO: 1.

93p. The KOD polymerase variant of any one of the preceding embodiments, comprising a T substitution at a position corresponding to D259 SEQ ID NO: 1.

93q. The KOD polymerase variant of any one of the preceding embodiments, comprising a V substitution at a position corresponding to D259 SEQ ID NO: 1.

93r. The KOD polymerase variant of any one of the preceding embodiments, comprising a W substitution at a position corresponding to D259 SEQ ID NO: 1.

93s. The KOD polymerase variant of any one of the preceding embodiments, comprising a Y substitution at a position corresponding to D259 SEQ ID NO: 1.

94. The KOD polymerase variant of any one of the preceding embodiments, comprising a W substitution at a position corresponding to L260 of SEQ ID NO: 1.

95. The KOD polymerase variant of any one of the preceding embodiments, comprising a D, E, or F substitution at a position corresponding to Y261 of SEQ ID NO: 1.

95b. The KOD polymerase variant of any one of the preceding embodiments, comprising a D substitution at a position corresponding to Y261 SEQ ID NO: 1.

95c. The KOD polymerase variant of any one of the preceding embodiments, comprising an E substitution at a position corresponding to Y261 SEQ ID NO: 1.

95d. The KOD polymerase variant of any one of the preceding embodiments, comprising a F substitution at a position corresponding to Y261 SEQ ID NO: 1.

96. The KOD polymerase variant of any one of the preceding embodiments, comprising a D or P substitution at a position corresponding to Y273 of SEQ ID NO: 1.

96b. The KOD polymerase variant of any one of the preceding embodiments, comprising a D substitution at a position corresponding to Y273 SEQ ID NO: 1.

96c. The KOD polymerase variant of any one of the preceding embodiments, comprising a P substitution at a position corresponding to Y273 SEQ ID NO: 1.

97. The KOD polymerase variant of any one of the preceding embodiments, comprising a D substitution at a position corresponding to T274 of SEQ ID NO: 1.

98. The KOD polymerase variant of any one of the preceding embodiments, comprising a H, P, R, or S substitution at a position corresponding to L275 of SEQ ID NO: 1.

98b. The KOD polymerase variant of any one of the preceding embodiments, comprising a H substitution at a position corresponding to L275 SEQ ID NO: 1.

98c. The KOD polymerase variant of any one of the preceding embodiments, comprising a P substitution at a position corresponding to L275 SEQ ID NO: 1.

98d. The KOD polymerase variant of any one of the preceding embodiments, comprising a R substitution at a position corresponding to L275 SEQ ID NO: 1.

98e. The KOD polymerase variant of any one of the preceding embodiments, comprising a S substitution at a position corresponding to L275 SEQ ID NO: 1.

99. The KOD polymerase variant of any one of the preceding embodiments, comprising an E or P substitution at a position corresponding to A277 of SEQ ID NO: 1.

99b. The KOD polymerase variant of any one of the preceding embodiments, comprising an E substitution at a position corresponding to A277 SEQ ID NO: 1.

99c. The KOD polymerase variant of any one of the preceding embodiments, comprising a P substitution at a position corresponding to A277 SEQ ID NO: 1.

100. The KOD polymerase variant of any one of the preceding embodiments, comprising a F substitution at a position corresponding to V278 of SEQ ID NO: 1.

101. The KOD polymerase variant of any one of the preceding embodiments, comprising a M substitution at a position corresponding to E280 of SEQ ID NO: 1.

102. The KOD polymerase variant of any one of the preceding embodiments, comprising a W substitution at a position corresponding to A281 of SEQ ID NO: 1.

103. The KOD polymerase variant of any one of the preceding embodiments, comprising a D, E, F, I, M, P, T, or V substitution at a position corresponding to K287 of SEQ ID NO: 1.

103b. The KOD polymerase variant of any one of the preceding embodiments, comprising a D substitution at a position corresponding to K287 SEQ ID NO: 1.

103c. The KOD polymerase variant of any one of the preceding embodiments, comprising an E substitution at a position corresponding to K287 SEQ ID NO: 1.

103d. The KOD polymerase variant of any one of the preceding embodiments, comprising a F substitution at a position corresponding to K287 SEQ ID NO: 1.

103e. The KOD polymerase variant of any one of the preceding embodiments, comprising an I substitution at a position corresponding to K287 SEQ ID NO: 1.

103f. The KOD polymerase variant of any one of the preceding embodiments, comprising a M substitution at a position corresponding to K287 SEQ ID NO: 1.

103g. The KOD polymerase variant of any one of the preceding embodiments, comprising a P substitution at a position corresponding to K287 SEQ ID NO: 1.

103h. The KOD polymerase variant of any one of the preceding embodiments, comprising a T substitution at a position corresponding to K287 SEQ ID NO: 1.

103i. The KOD polymerase variant of any one of the preceding embodiments, comprising a V substitution at a position corresponding to K287 SEQ ID NO: 1.

104. The KOD polymerase variant of any one of the preceding embodiments, comprising an A, D, E, F, G, H, I, M, P, Q, R, S, T, V, W, or Y substitution at a position corresponding to K289 of SEQ ID NO: 1.

104b. The KOD polymerase variant of any one of the preceding embodiments, comprising an A substitution at a position corresponding to K289 SEQ ID NO: 1.

104c. The KOD polymerase variant of any one of the preceding embodiments, comprising a D substitution at a position corresponding to K289 SEQ ID NO: 1.

104d. The KOD polymerase variant of any one of the preceding embodiments, comprising an E substitution at a position corresponding to K289 SEQ ID NO: 1.

104e. The KOD polymerase variant of any one of the preceding embodiments, comprising a F substitution at a position corresponding to K289 SEQ ID NO: 1.

104f. The KOD polymerase variant of any one of the preceding embodiments, comprising a G substitution at a position corresponding to K289 SEQ ID NO: 1.

104g. The KOD polymerase variant of any one of the preceding embodiments, comprising a H substitution at a position corresponding to K289 SEQ ID NO: 1.

104h. The KOD polymerase variant of any one of the preceding embodiments, comprising an I substitution at a position corresponding to K289 SEQ ID NO: 1.

104i. The KOD polymerase variant of any of the preceding embodiments, comprising a L substitution at a position corresponding to K289 SEQ ID NO: 1.

104j. The KOD polymerase variant of any one of the preceding embodiments, comprising a M substitution at a position corresponding to K289 SEQ ID NO: 1.

104k. The KOD polymerase variant of any one of the preceding embodiments, comprising a P substitution at a position corresponding to K289 SEQ ID NO: 1.

104l. The KOD polymerase variant of any one of the preceding embodiments, comprising a Q substitution at a position corresponding to K289 SEQ ID NO: 1.

104m. The KOD polymerase variant of any one of the preceding embodiments, comprising a R substitution at a position corresponding to K289 SEQ ID NO: 1.

104n. The KOD polymerase variant of any one of the preceding embodiments, comprising a S substitution at a position corresponding to K289 SEQ ID NO: 1.

104o. The KOD polymerase variant of any one of the preceding embodiments, comprising a T substitution at a position corresponding to K289 SEQ ID NO: 1.

104p. The KOD polymerase variant of any one of the preceding embodiments, comprising a V substitution at a position corresponding to K289 SEQ ID NO: 1.

104q. The KOD polymerase variant of any one of the preceding embodiments, comprising a W substitution at a position corresponding to K289 SEQ ID NO: 1.

104r. The KOD polymerase variant of any one of the preceding embodiments, comprising a Y substitution at a position corresponding to K289 SEQ ID NO: 1.

105. The KOD polymerase variant of any one of the preceding embodiments, comprising an A, D, E, G, H, K, N, P, Q, R, S, or T substitution at a position corresponding to V290 of SEQ ID NO: 1.

105b. The KOD polymerase variant of any one of the preceding embodiments, comprising an A substitution at a position corresponding to V290 SEQ ID NO: 1.

105c. The KOD polymerase variant of any one of the preceding embodiments, comprising a D substitution at a position corresponding to V290 SEQ ID NO: 1.

105d. The KOD polymerase variant of any one of the preceding embodiments, comprising an E substitution at a position corresponding to V290 SEQ ID NO: 1.

105e. The KOD polymerase variant of any one of the preceding embodiments, comprising a G substitution at a position corresponding to V290 SEQ ID NO: 1.

105f. The KOD polymerase variant of any one of the preceding embodiments, comprising a H substitution at a position corresponding to V290 SEQ ID NO: 1.

105g. The KOD polymerase variant of any one of the preceding embodiments, comprising a K substitution at a position corresponding to V290 SEQ ID NO: 1.

105h. The KOD polymerase variant of any one of the preceding embodiments, comprising a N substitution at a position corresponding to V290 SEQ ID NO: 1.

105i. The KOD polymerase variant of any one of the preceding embodiments, comprising a P substitution at a position corresponding to V290 SEQ ID NO: 1.

105j. The KOD polymerase variant of any one of the preceding embodiments, comprising a Q substitution at a position corresponding to V290 SEQ ID NO: 1.

105k. The KOD polymerase variant of any one of the preceding embodiments, comprising a R substitution at a position corresponding to V290 SEQ ID NO: 1.

105l. The KOD polymerase variant of any one of the preceding embodiments, comprising a S substitution at a position corresponding to V290 SEQ ID NO: 1.

105m. The KOD polymerase variant of any one of the preceding embodiments, comprising a T substitution at a position corresponding to V290 SEQ ID NO: 1.

106. The KOD polymerase variant of any one of the preceding embodiments, comprising a D, E, N, Q, or T substitution at a position corresponding to A292 of SEQ ID NO: 1.

106b. The KOD polymerase variant of any one of the preceding embodiments, comprising a D substitution at a position corresponding to A292 SEQ ID NO: 1.

106c. The KOD polymerase variant of any one of the preceding embodiments, comprising an E substitution at a position corresponding to A292 SEQ ID NO: 1.

106d. The KOD polymerase variant of any one of the preceding embodiments, comprising a N substitution at a position corresponding to A292 SEQ ID NO: 1.

106e. The KOD polymerase variant of any one of the preceding embodiments, comprising a Q substitution at a position corresponding to A292 SEQ ID NO: 1.

106f. The KOD polymerase variant of any one of the preceding embodiments, comprising a T substitution at a position corresponding to A292 SEQ ID NO: 1.

107. The KOD polymerase variant of any one of the preceding embodiments, comprising a K, P, or R substitution at a position corresponding to E294 of SEQ ID NO: 1.

107b. The KOD polymerase variant of any one of the preceding embodiments, comprising a K substitution at a position corresponding to E294 SEQ ID NO: 1.

107c. The KOD polymerase variant of any one of the preceding embodiments, comprising a P substitution at a position corresponding to E294 SEQ ID NO: 1.

107d. The KOD polymerase variant of any one of the preceding embodiments, comprising a R substitution at a position corresponding to E294 SEQ ID NO: 1.

108. The KOD polymerase variant of any one of the preceding embodiments, comprising an A, C, D, E, G, H, K, N, P, Q, R, S, T, W, or Y substitution at a position corresponding to I295 of SEQ ID NO: 1.

108b. The KOD polymerase variant of any one of the preceding embodiments, comprising an A substitution at a position corresponding to I295 SEQ ID NO: 1.

108c. The KOD polymerase variant of any one of the preceding embodiments, comprising a C substitution at a position corresponding to I295 SEQ ID NO: 1.

108d. The KOD polymerase variant of any one of the preceding embodiments, comprising a D substitution at a position corresponding to I295 SEQ ID NO: 1.

108e. The KOD polymerase variant of any one of the preceding embodiments, comprising an E substitution at a position corresponding to I295 SEQ ID NO: 1.

108f. The KOD polymerase variant of any one of the preceding embodiments, comprising a G substitution at a position corresponding to I295 SEQ ID NO: 1.

108g. The KOD polymerase variant of any one of the preceding embodiments, comprising a H substitution at a position corresponding to I295 SEQ ID NO: 1.

108h. The KOD polymerase variant of any one of the preceding embodiments, comprising a K substitution at a position corresponding to I295 SEQ ID NO: 1.

108i. The KOD polymerase variant of any one of the preceding embodiments, comprising a N substitution at a position corresponding to I295 SEQ ID NO: 1.

108j. The KOD polymerase variant of any one of the preceding embodiments, comprising a P substitution at a position corresponding to I295 SEQ ID NO: 1.

108k. The KOD polymerase variant of any one of the preceding embodiments, comprising a Q substitution at a position corresponding to I295 SEQ ID NO: 1.

108l. The KOD polymerase variant of any one of the preceding embodiments, comprising a R substitution at a position corresponding to I295 SEQ ID NO: 1.

108m. The KOD polymerase variant of any one of the preceding embodiments, comprising a S substitution at a position corresponding to I295 SEQ ID NO: 1.

108n. The KOD polymerase variant of any one of the preceding embodiments, comprising a T substitution at a position corresponding to I295 SEQ ID NO: 1.

108o. The KOD polymerase variant of any one of the preceding embodiments, comprising a W substitution at a position corresponding to I295 SEQ ID NO: 1.

108p. The KOD polymerase variant of any one of the preceding embodiments, comprising a Y substitution at a position corresponding to I295 SEQ ID NO: 1.

109. The KOD polymerase variant of any one of the preceding embodiments, comprising a D or P substitution at a position corresponding to A298 of SEQ ID NO: 1.

109b. The KOD polymerase variant of any one of the preceding embodiments, comprising a D substitution at a position corresponding to A298 SEQ ID NO: 1.

109c. The KOD polymerase variant of any one of the preceding embodiments, comprising a P substitution at a position corresponding to A298 SEQ ID NO: 1.

110. The KOD polymerase variant of any one of the preceding embodiments, comprising a D, E, G, H, I, K, L, M, N, P, Q, R, T, or V substitution at a position corresponding to W299 of SEQ ID NO: 1.

110b. The KOD polymerase variant of any one of the preceding embodiments, comprising a D substitution at a position corresponding to W299 SEQ ID NO: 1.

110c. The KOD polymerase variant of any one of the preceding embodiments, comprising an E substitution at a position corresponding to W299 SEQ ID NO: 1.

110d. The KOD polymerase variant of any one of the preceding embodiments, comprising a G substitution at a position corresponding to W299 SEQ ID NO: 1.

110e. The KOD polymerase variant of any one of the preceding embodiments, comprising a H substitution at a position corresponding to W299 SEQ ID NO: 1.

110f. The KOD polymerase variant of any one of the preceding embodiments, comprising an I substitution at a position corresponding to W299 SEQ ID NO: 1.

110g. The KOD polymerase variant of any one of the preceding embodiments, comprising a K substitution at a position corresponding to W299 SEQ ID NO: 1.

110h. The KOD polymerase variant of any one of the preceding embodiments, comprising a L substitution at a position corresponding to W299 SEQ ID NO: 1.

110i. The KOD polymerase variant of any one of the preceding embodiments, comprising a M substitution at a position corresponding to W299 SEQ ID NO: 1.

110j. The KOD polymerase variant of any one of the preceding embodiments, comprising a N substitution at a position corresponding to W299 SEQ ID NO: 1.

110k. The KOD polymerase variant of any one of the preceding embodiments, comprising a P substitution at a position corresponding to W299 SEQ ID NO: 1.

110l. The KOD polymerase variant of any one of the preceding embodiments, comprising a Q substitution at a position corresponding to W299 SEQ ID NO: 1.

110m. The KOD polymerase variant of any one of the preceding embodiments, comprising a R substitution at a position corresponding to W299 SEQ ID NO: 1.

110n. The KOD polymerase variant of any one of the preceding embodiments, comprising a T substitution at a position corresponding to W299 SEQ ID NO: 1.

110o. The KOD polymerase variant of any one of the preceding embodiments, comprising a V substitution at a position corresponding to W299 SEQ ID NO: 1.

111. The KOD polymerase variant of any one of the preceding embodiments, comprising a P substitution at a position corresponding to E300 of SEQ ID NO: 1.

112. The KOD polymerase variant of any one of the preceding embodiments, comprising a P substitution at a position corresponding to T301 of SEQ ID NO: 1.

113. The KOD polymerase variant of any one of the preceding embodiments, comprising a P substitution at a position corresponding to G302 of SEQ ID NO: 1.

114. The KOD polymerase variant of any one of the preceding embodiments, comprising a H substitution at a position corresponding to L305 of SEQ ID NO: 1.

115. The KOD polymerase variant of any one of the preceding embodiments, comprising a D, F, H, K, L, N, Q, R, W, or Y substitution at a position corresponding to V308 of SEQ ID NO: 1.

115b. The KOD polymerase variant of any one of the preceding embodiments, comprising a D substitution at a position corresponding to V308 SEQ ID NO: 1.

115c. The KOD polymerase variant of any one of the preceding embodiments, comprising a F substitution at a position corresponding to V308 SEQ ID NO: 1.

115d. The KOD polymerase variant of any one of the preceding embodiments, comprising a H substitution at a position corresponding to V308 SEQ ID NO: 1.

115e. The KOD polymerase variant of any one of the preceding embodiments, comprising a K substitution at a position corresponding to V308 SEQ ID NO: 1.

115f. The KOD polymerase variant of any one of the preceding embodiments, comprising a L substitution at a position corresponding to V308 SEQ ID NO: 1.

115g. The KOD polymerase variant of any one of the preceding embodiments, comprising a N substitution at a position corresponding to V308 SEQ ID NO: 1.

115h. The KOD polymerase variant of any one of the preceding embodiments, comprising a Q substitution at a position corresponding to V308 SEQ ID NO: 1.

115i. The KOD polymerase variant of any one of the preceding embodiments, comprising a R substitution at a position corresponding to V308 SEQ ID NO: 1.

115j. The KOD polymerase variant of any one of the preceding embodiments, comprising a W substitution at a position corresponding to V308 SEQ ID NO: 1.

115k. The KOD polymerase variant of any one of the preceding embodiments, comprising a Y substitution at a position corresponding to V308 SEQ ID NO: 1.

116. The KOD polymerase variant of any one of the preceding embodiments, comprising a D, P, or R substitution at a position corresponding to A309 of SEQ ID NO: 1.

116b. The KOD polymerase variant of any one of the preceding embodiments, comprising a D substitution at a position corresponding to A309 SEQ ID NO: 1.

116c. The KOD polymerase variant of any one of the preceding embodiments, comprising a P substitution at a position corresponding to A309 SEQ ID NO: 1.

116d. The KOD polymerase variant of any one of the preceding embodiments, comprising a R substitution at a position corresponding to A309 SEQ ID NO: 1.

117. The KOD polymerase variant of any one of the preceding embodiments, comprising an A, C, D, E, F, G, H, I, K, L, M, N, P, Q, R, S, T, V, or W substitution at a position corresponding to Y311 of SEQ ID NO: 1.

117b. The KOD polymerase variant of any one of the preceding embodiments, comprising an A substitution at a position corresponding to Y311 SEQ ID NO: 1.

117c. The KOD polymerase variant of any one of the preceding embodiments, comprising a C substitution at a position corresponding to Y311 SEQ ID NO: 1.

117d. The KOD polymerase variant of any one of the preceding embodiments, comprising a D substitution at a position corresponding to Y311 SEQ ID NO: 1.

117e. The KOD polymerase variant of any one of the preceding embodiments, comprising an E substitution at a position corresponding to Y311 SEQ ID NO: 1.

117f. The KOD polymerase variant of any one of the preceding embodiments, comprising a F substitution at a position corresponding to Y311 SEQ ID NO: 1.

117g. The KOD polymerase variant of any one of the preceding embodiments, comprising a G substitution at a position corresponding to Y311 SEQ ID NO: 1.

117h. The KOD polymerase variant of any one of the preceding embodiments, comprising a H substitution at a position corresponding to Y311 SEQ ID NO: 1.

117i. The KOD polymerase variant of any one of the preceding embodiments, comprising an I substitution at a position corresponding to Y311 SEQ ID NO: 1.

117j. The KOD polymerase variant of any one of the preceding embodiments, comprising a K substitution at a position corresponding to Y311 SEQ ID NO: 1.

117k. The KOD polymerase variant of any one of the preceding embodiments, comprising a L substitution at a position corresponding to Y311 SEQ ID NO: 1.

117l. The KOD polymerase variant of any one of the preceding embodiments, comprising a M substitution at a position corresponding to Y311 SEQ ID NO: 1.

117m. The KOD polymerase variant of any one of the preceding embodiments, comprising a N substitution at a position corresponding to Y311 SEQ ID NO: 1.

117n. The KOD polymerase variant of any one of the preceding embodiments, comprising a P substitution at a position corresponding to Y311 SEQ ID NO: 1.

117o. The KOD polymerase variant of any one of the preceding embodiments, comprising a Q substitution at a position corresponding to Y311 SEQ ID NO: 1.

117p. The KOD polymerase variant of any one of the preceding embodiments, comprising a R substitution at a position corresponding to Y311 SEQ ID NO: 1.

117q. The KOD polymerase variant of any one of the preceding embodiments, comprising a S substitution at a position corresponding to Y311 SEQ ID NO: 1.

117r. The KOD polymerase variant of any one of the preceding embodiments, comprising a T substitution at a position corresponding to Y311 SEQ ID NO: 1.

117s. The KOD polymerase variant of any one of the preceding embodiments, comprising a V substitution at a position corresponding to Y311 SEQ ID NO: 1.

117t. The KOD polymerase variant of any one of the preceding embodiments, comprising a W substitution at a position corresponding to Y311 SEQ ID NO: 1.

118. The KOD polymerase variant of any one of the preceding embodiments, comprising a D, E, F, H, I, K, L, M, N, P, Q, R, V, W, or Y substitution at a position corresponding to S312 of SEQ ID NO: 1.

118b. The KOD polymerase variant of any one of the preceding embodiments, comprising a D substitution at a position corresponding to S312 SEQ ID NO: 1.

118c. The KOD polymerase variant of any one of the preceding embodiments, comprising an E substitution at a position corresponding to S312 SEQ ID NO: 1.

118d. The KOD polymerase variant of any one of the preceding embodiments, comprising a F substitution at a position corresponding to S312 SEQ ID NO: 1.

118e. The KOD polymerase variant of any one of the preceding embodiments, comprising a H substitution at a position corresponding to S312 SEQ ID NO: 1.

118f. The KOD polymerase variant of any one of the preceding embodiments, comprising an I substitution at a position corresponding to S312 SEQ ID NO: 1.

118g. The KOD polymerase variant of any one of the preceding embodiments, comprising a K substitution at a position corresponding to S312 SEQ ID NO: 1.

118h. The KOD polymerase variant of any one of the preceding embodiments, comprising a L substitution at a position corresponding to S312 SEQ ID NO: 1.

118i. The KOD polymerase variant of any one of the preceding embodiments, comprising a M substitution at a position corresponding to S312 SEQ ID NO: 1.

118j. The KOD polymerase variant of any one of the preceding embodiments, comprising a N substitution at a position corresponding to S312 SEQ ID NO: 1.

118k. The KOD polymerase variant of any one of the preceding embodiments, comprising a P substitution at a position corresponding to S312 SEQ ID NO: 1.

118l. The KOD polymerase variant of any one of the preceding embodiments, comprising a Q substitution at a position corresponding to S312 SEQ ID NO: 1.

118m. The KOD polymerase variant of any one of the preceding embodiments, comprising a R substitution at a position corresponding to S312 SEQ ID NO: 1.

118n. The KOD polymerase variant of any one of the preceding embodiments, comprising a V substitution at a position corresponding to S312 SEQ ID NO: 1.

118o. The KOD polymerase variant of any one of the preceding embodiments, comprising a W substitution at a position corresponding to S312 SEQ ID NO: 1.

118p. The KOD polymerase variant of any one of the preceding embodiments, comprising a Y substitution at a position corresponding to S312 SEQ ID NO: 1.

119. The KOD polymerase variant of any one of the preceding embodiments, comprising a P or Q substitution at a position corresponding to M313 of SEQ ID NO: 1.

119b. The KOD polymerase variant of any one of the preceding embodiments, comprising a P substitution at a position corresponding to M313 SEQ ID NO: 1.

119c. The KOD polymerase variant of any one of the preceding embodiments, comprising a Q substitution at a position corresponding to M313 SEQ ID NO: 1.

120. The KOD polymerase variant of any one of the preceding embodiments, comprising a P substitution at a position corresponding to E314 of SEQ ID NO: 1.

121. The KOD polymerase variant of any one of the preceding embodiments, comprising an A, C, F, G, I, K, L, M, N, P, Q, R, S, T, V, W, or Y substitution at a position corresponding to D315 of SEQ ID NO: 1.

121b. The KOD polymerase variant of any one of the preceding embodiments, comprising an A substitution at a position corresponding to D315 SEQ ID NO: 1.

121c. The KOD polymerase variant of any one of the preceding embodiments, comprising a C substitution at a position corresponding to D315 SEQ ID NO: 1.

121d. The KOD polymerase variant of any one of the preceding embodiments, comprising a F substitution at a position corresponding to D315 SEQ ID NO: 1.

121e. The KOD polymerase variant of any one of the preceding embodiments, comprising a G substitution at a position corresponding to D315 SEQ ID NO: 1.

121f. The KOD polymerase variant of any one of the preceding embodiments, comprising an I substitution at a position corresponding to D315 SEQ ID NO: 1.

121g. The KOD polymerase variant of any one of the preceding embodiments, comprising a K substitution at a position corresponding to D315 SEQ ID NO: 1.

121h. The KOD polymerase variant of any one of the preceding embodiments, comprising a L substitution at a position corresponding to D315 SEQ ID NO: 1.

121i. The KOD polymerase variant of any one of the preceding embodiments, comprising a M substitution at a position corresponding to D315 SEQ ID NO: 1.

121j. The KOD polymerase variant of any one of the preceding embodiments, comprising a N substitution at a position corresponding to D315 SEQ ID NO: 1.

121k. The KOD polymerase variant of any one of the preceding embodiments, comprising a P substitution at a position corresponding to D315 SEQ ID NO: 1.

121l. The KOD polymerase variant of any one of the preceding embodiments, comprising a Q substitution at a position corresponding to D315 SEQ ID NO: 1.

121m. The KOD polymerase variant of any one of the preceding embodiments, comprising a R substitution at a position corresponding to D315 SEQ ID NO: 1.

121n. The KOD polymerase variant of any one of the preceding embodiments, comprising a S substitution at a position corresponding to D315 SEQ ID NO: 1.

121o. The KOD polymerase variant of any one of the preceding embodiments, comprising a T substitution at a position corresponding to D315 SEQ ID NO: 1.

121p. The KOD polymerase variant of any one of the preceding embodiments, comprising a V substitution at a position corresponding to D315 SEQ ID NO: 1.

121q. The KOD polymerase variant of any one of the preceding embodiments, comprising a W substitution at a position corresponding to D315 SEQ ID NO: 1.

121r. The KOD polymerase variant of any one of the preceding embodiments, comprising a Y substitution at a position corresponding to D315 SEQ ID NO: 1.

122. The KOD polymerase variant of any one of the preceding embodiments, comprising a D, E, F, H, M, N, Q, R, W, or Y substitution at a position corresponding to A316 of SEQ ID NO: 1.

122b. The KOD polymerase variant of any one of the preceding embodiments, comprising a D substitution at a position corresponding to A316 SEQ ID NO: 1.

122c. The KOD polymerase variant of any one of the preceding embodiments, comprising an E substitution at a position corresponding to A316 SEQ ID NO: 1.

122d. The KOD polymerase variant of any one of the preceding embodiments, comprising a F substitution at a position corresponding to A316 SEQ ID NO: 1.

122e. The KOD polymerase variant of any one of the preceding embodiments, comprising a H substitution at a position corresponding to A316 SEQ ID NO: 1.

122f. The KOD polymerase variant of any one of the preceding embodiments, comprising a M substitution at a position corresponding to A316 SEQ ID NO: 1.

122g. The KOD polymerase variant of any one of the preceding embodiments, comprising a N substitution at a position corresponding to A316 SEQ ID NO: 1.

122h. The KOD polymerase variant of any one of the preceding embodiments, comprising a Q substitution at a position corresponding to A316 SEQ ID NO: 1.

122i. The KOD polymerase variant of any one of the preceding embodiments, comprising a R substitution at a position corresponding to A316 SEQ ID NO: 1.

122j. The KOD polymerase variant of any one of the preceding embodiments, comprising a W substitution at a position corresponding to A316 SEQ ID NO: 1.

122k. The KOD polymerase variant of any one of the preceding embodiments, comprising a Y substitution at a position corresponding to A316 SEQ ID NO: 1.

123. The KOD polymerase variant of any one of the preceding embodiments, comprising a P substitution at a position corresponding to K317 of SEQ ID NO: 1.

124. The KOD polymerase variant of any one of the preceding embodiments, comprising a P or R substitution at a position corresponding to V318 of SEQ ID NO: 1.

124b. The KOD polymerase variant of any one of the preceding embodiments, comprising a P substitution at a position corresponding to V318 SEQ ID NO: 1.

124c. The KOD polymerase variant of any one of the preceding embodiments, comprising a R substitution at a position corresponding to V318 SEQ ID NO: 1.

125. The KOD polymerase variant of any one of the preceding embodiments, comprising a D, I, K, L, M, Q, R, or V substitution at a position corresponding to T319 of SEQ ID NO: 1.

125b. The KOD polymerase variant of any one of the preceding embodiments, comprising a D substitution at a position corresponding to T319 SEQ ID NO: 1.

125c. The KOD polymerase variant of any one of the preceding embodiments, comprising an I substitution at a position corresponding to T319 SEQ ID NO: 1.

125d. The KOD polymerase variant of any one of the preceding embodiments, comprising a K substitution at a position corresponding to T319 SEQ ID NO: 1.

125e. The KOD polymerase variant of any one of the preceding embodiments, comprising a L substitution at a position corresponding to T319 SEQ ID NO: 1.

125f. The KOD polymerase variant of any one of the preceding embodiments, comprising a M substitution at a position corresponding to T319 SEQ ID NO: 1.

125g. The KOD polymerase variant of any one of the preceding embodiments, comprising a Q substitution at a position corresponding to T319 SEQ ID NO: 1.

125h. The KOD polymerase variant of any one of the preceding embodiments, comprising a R substitution at a position corresponding to T319 SEQ ID NO: 1.

125i. The KOD polymerase variant of any one of the preceding embodiments, comprising a V substitution at a position corresponding to T319 SEQ ID NO: 1.

126. A method for amplifying a DNA template wherein the DNA template comprises one or more uracil and/or inosine nucleotides, the method comprising amplifying the DNA template using the KOD polymerase variant of any one of embodiments 1-125i.

127. The method of embodiment 126, wherein the KOD polymerase variant is the KOD polymerase variant of any one of embodiments 1-125i.

128. A method for amplifying a DNA template to create a synthesized DNA strand in which synthesized DNA strand comprises one or more uracil residues, the method comprising inserting one or more uracil nucleotides into the synthesized DNA strand using the KOD polymerase variant of any one of embodiments 1-125i.

129. The method of embodiment 128, wherein the KOD polymerase variant is according to any one of embodiments 1-125i.

130. A method for amplifying a DNA template to create a synthesized DNA strand in which the DNA template comprises one or more uracil and/or inosine nucleotides and/or the synthesized DNA strand comprises one or more uracil residues, the method comprising amplifying the DNA template and/or inserting one or more uracil nucleotides into the synthesized DNA strand using the KOD polymerase variant of any one of embodiments 1-125i.

131. A method for amplifying a DNA template to create a synthesized DNA strand in which the DNA template comprises one or more uracil and/or inosine nucleotides and/or the synthesized DNA strand comprises one or more uracil residues, the method comprising amplifying the DNA template and/or inserting one or more uracil nucleotides into the synthesized DNA strand using the KOD polymerase variant of any one of embodiments 1-125i.

132. The method of the embodiment 131, wherein the KOD polymerase variant is according to any one of embodiments 1-125i.

133. A Thermococcus kodakarensis (KOD) polymerase variant, comprising:
a W at a position corresponding to I16 of SEQ ID NO: 1;
an F at a position corresponding to P90 of SEQ ID NO: 1;
a W at a position corresponding to P90 of SEQ ID NO: 1;
a P at a position corresponding to F116 of SEQ ID NO: 1;
an M at a position corresponding to A117 of SEQ ID NO: 1;
a Q at a position corresponding to R119 of SEQ ID NO: 1;
an I at a position corresponding to D123 of SEQ ID NO: 1;
an M at a position corresponding to D215 of SEQ ID NO: 1;
a W at a position corresponding to I16 of SEQ ID NO: 1 and a P at a position corresponding to F116 of SEQ ID NO: 1;
an F at a position corresponding to P90 of SEQ ID NO: 1, a P at a position corresponding to F116 of SEQ ID NO: 1, and an M at a position corresponding to A117 of SEQ ID NO: 1;
a W at a position corresponding to I16 of SEQ ID NO: 1, an H at a position corresponding to P36 of SEQ ID NO: 1, and a P at a position corresponding to F116 of SEQ ID NO: 1;
an L at a position corresponding to Y7 of SEQ ID NO: 1, an F at a position corresponding to I16 of SEQ ID NO: 1, and an I at a position corresponding to V93 of SEQ ID NO: 1; or
an M at a position corresponding to Y7 of SEQ ID NO: 1 and an L at a position corresponding to I16 of SEQ ID NO: 1.

134. The KOD polymerase variant of embodiment 133, comprising:
a W at a position corresponding to I16 of SEQ ID NO: 1.

135. The KOD polymerase variant of embodiment 133, comprising:
an F at a position corresponding to P90 of SEQ ID NO: 1.

136. The KOD polymerase variant of embodiment 133, comprising:
a W at a position corresponding to P90 of SEQ ID NO: 1.

137. The KOD polymerase variant of embodiment 133, comprising:
a P at a position corresponding to F116 of SEQ ID NO: 1.

138. The KOD polymerase variant of embodiment 133, comprising:
an M at a position corresponding to A117 of SEQ ID NO: 1.

139. The KOD polymerase variant of embodiment 133, comprising:
a Q at a position corresponding to R119 of SEQ ID NO: 1.

140. The KOD polymerase variant of embodiment 133, comprising:
an I at a position corresponding to D123 of SEQ ID NO: 1.

141. The KOD polymerase variant of embodiment 133, comprising:
an M at a position corresponding to D215 of SEQ ID NO: 1.

142. The KOD polymerase variant of embodiment 133, comprising:
a W at a position corresponding to I16 of SEQ ID NO: 1 and a P at a position corresponding to F116 of SEQ ID NO: 1.

143. The KOD polymerase variant of embodiment 133, comprising:
an F at a position corresponding to P90 of SEQ ID NO: 1, a P at a position corresponding to F116 of SEQ ID NO: 1, and an M at a position corresponding to A117 of SEQ ID NO: 1.

144. The KOD polymerase variant of embodiment 133, comprising:
a W at a position corresponding to I16 of SEQ ID NO: 1, an H at a position corresponding to P36 of SEQ ID NO: 1, and a P at a position corresponding to F116 of SEQ ID NO: 1.

145. The KOD polymerase variant of embodiment 133, comprising:
an L at a position corresponding to Y7 of SEQ ID NO: 1, an F at a position corresponding to I16 of SEQ ID NO: 1, and an I at a position corresponding to V93 of SEQ ID NO: 1.

146. The KOD polymerase variant of embodiment 133, comprising:
an M at a position corresponding to Y7 of SEQ ID NO: 1 and an L at a position corresponding to I16 of SEQ ID NO: 1.

1. A Thermococcus kodakarensis (KOD) polymerase variant, comprising one or more of the following substitutions:
an aspartate (D), glutamate (E), phenylalanine (F), histidine (H), lysine (K), leucine (L), asparagine (N), threonine (T), valine (V), or tryptophan (W) at a position corresponding to isoleucine 2 (I2) of SEQ ID NO: 1;
a D, H, I, serine (S), T, or tyrosine (Y) at a position corresponding to L3 of SEQ ID NO: 1;
an I, L, methionine (M), proline (P), or Y at a position corresponding to D4 of SEQ ID NO: 1;
a D, E, F, glycine (G), H, L, W, or Y at a position corresponding to T5 of SEQ ID NO: 1;
a L at a position corresponding to D6 of SEQ ID NO: 1;
a D, E, H, K, N, glutamine (Q), arginine (R), S, or T at a position corresponding to Y7 of SEQ ID NO: 1;
a D, E, H, or T at a position corresponding to I8 of SEQ ID NO: 1;
an alanine (A), cysteine (C), D, E, F, H, I, L, N, R, or Y at a position corresponding to T9 of SEQ ID NO: 1;
a P or Q at a position corresponding to E10 of SEQ ID NO: 1;
a Q or T at a position corresponding to D1I of SEQ ID NO: 1;
a C, Q, T, or V at a position corresponding to G12 of SEQ ID NO: 1;
a G, H, T, V, or W at a position corresponding to K13 of SEQ ID NO: 1;
a C, D, E, F, H, M, Q, S, T or W at a position corresponding to P14 of SEQ ID NO: 1;
a Q or W at a position corresponding to V15 of SEQ ID NO: 1;
a P at a position corresponding to R17 of SEQ ID NO: 1;
a Y at a position corresponding to I18 of SEQ ID NO: 1;
a W at a position corresponding to E22 of SEQ ID NO: 1;
a D at a position corresponding to F34 of SEQ ID NO: 1;
a C or T at a position corresponding to P36 of SEQ ID NO: 1;
an A, D, E, F, G, S, or T at a position corresponding to Y37 of SEQ ID NO: 1;
a L at a position corresponding to V71 of SEQ ID NO: 1;
a R at a position corresponding to F87 of SEQ ID NO: 1;
a D, P, or W at a position corresponding to H89 of SEQ ID NO: 1;
a G at a position corresponding to P90 of SEQ ID NO: 1;
a D, E, N, or Y at a position corresponding to Q91 of SEQ ID NO: 1;
a P at a position corresponding to D92 of SEQ ID NO: 1;
a C, D, E, F, G, H, I, M, N, Q, W, or Y at a position corresponding to P94 of SEQ ID NO: 1;
a P and R at a position corresponding to I96 of SEQ ID NO: 1;
an A, C, D, E, F, G, H, I, K, L, M, N, P, Q, S, T, V, W, or Y at a position corresponding to R97 of SEQ ID NO: 1;
an E or Q at a position corresponding to R101 of SEQ ID NO: 1;
a D or F at a position corresponding to I109 of SEQ ID NO: 1;
a P at a position corresponding to Y110 of SEQ ID NO: 1;
a G at a position corresponding to E111 of SEQ ID NO: 1;
a D or E at a position corresponding to Y112 of SEQ ID NO: 1;
a P at a position corresponding to D113 of SEQ ID NO: 1;
an E, G, H, K, N, P, Q, S, or Y at a position corresponding to I114 of SEQ ID NO: 1;
a C, D, F, H, N, S, T, or Y at a position corresponding to P115 of SEQ ID NO: 1;
an A, D, E, G, H, I, K, N, P, Q, R, or V at a position corresponding to F116 of SEQ ID NO: 1;
a D, E, L, M, Q, or W at a position corresponding to A117 of SEQ ID NO: 1;
an A, C, D, E, F, G, H, I, K, L, M, N, Q, S, T, V, W, or Y at a position corresponding to R119 of SEQ ID NO: 1;
a P at a position corresponding to Y120 of SEQ ID NO: 1;
a K, R, or W at a position corresponding to I122 of SEQ ID NO: 1; or
an A, C, F, H, I, K, L, M, Q, R, S, T, V, W, or a Y at a position corresponding to D123 of SEQ ID NO: 1.

2. (canceled)

3. The KOD polymerase variant of claim 1, further comprising one or more of the following substitutions:
an A, C, F, G, I, L, M, P, V, or W at a position corresponding to Y7 of SEQ ID NO: 1;
an A, C, F, H, K, S, W, or Y at a position corresponding to I16 of SEQ ID NO: 1;
a D, E, F, H, I, K, L, M, N, Q, R, V, W, or Y at a position corresponding to P36 of SEQ ID NO: 1;
a C, D, E, F, H, I, K, L, M, N, Q, R, W, or Y at a position corresponding to P90 of SEQ ID NO: 1; or
an A, C, D, E, F, G, H, K, M, N, P, Q, R, S, T, W, or Y at a position corresponding to V93 of SEQ ID NO: 1.

4.-6. (canceled)

7. The KOD polymerase variant of claim 1 comprising an amino acid sequence having at least 95% identity to SEQ ID NO: 1.

8.-19. (canceled)

20. The KOD polymerase variant of claim 1, comprising an amino acid sequence of any one of SEQ ID NOs: 741-1509 or 1511-3418.

21.-35. (canceled)

36. The KOD polymerase variant of claim 1, comprising the amino acid sequence of SEQ ID NO: 881, SEQ ID NO: 3225, SEQ ID NO: 3237, or SEQ ID NO: 3333.

37-40. (canceled)

41. A Thermococcus kodakarensis (KOD) polymerase variant, comprising one or more of the following substitutions:
a D at position Y7 of SEQ ID NO: 1;
an E at position P14 of SEQ ID NO: 1;
a L or M at position I16 of SEQ ID NO: 1;
an A or S at position P36 of SEQ ID NO: 1;
a D at position Y37 of SEQ ID NO: 1;
a D at position H89 of SEQ ID NO: 1;
a S at position P90 of SEQ ID NO: 1;
an I at position V93 of SEQ ID NO: 1;
a D at position P94 of SEQ ID NO: 1;
an A, F, H, P, or W at position R97 of SEQ ID NO: 1;
a P at position I114 of SEQ ID NO: 1;
a P at position F116 of SEQ ID NO: 1;
a M at position A117 of SEQ ID NO: 1;
an E, M, P, Q, or Y at position R119 of SEQ ID NO: 1; or
an I at position D123 of SEQ ID NO: 1.

42. The KOD polymerase variant of claim 41 further comprising one or more of the following substitutions:
a C, L, M, or P at position Y7 of SEQ ID NO: 1;
a F, H, or W at position I16 of SEQ ID NO: 1;
a H, R, or W at position P36 of SEQ ID NO: 1;
a F, I, W, or Y at position P90 of SEQ ID NO:1; or
an E, K, Q, or R at position V93 of SEQ ID NO: 1.

43. The KOD polymerase variant of claim 42 comprising an I at position V93 of SEQ ID NO: 1.

44. The KOD polymerase variant of claim 42 comprising a P at position F116 of SEQ ID NO: 1.

45. The KOD polymerase variant of claim 42 comprising a M at position A117 of SEQ ID NO: 1.

46. The KOD polymerase variant of claim 42 comprising a F at position P90 of SEQ ID NO: 1.

47. The KOD polymerase variant of claim 42 comprising one or more of the following substitutions:
a L or M at position Y7 of SEQ ID NO: 1;
a F, L, or W at position I16 of SEQ ID NO: 1;
a H at position P36 of SEQ ID NO: 1;
a F at position P90 of SEQ ID NO: 1;
an I or K at position V93 of SEQ ID NO: 1;
a D at position P94 of SEQ ID NO: 1;
an A or F at position R97 of SEQ ID NO: 1;
a P at position F116 of SEQ ID NO: 1;
a M at position A117 of SEQ ID NO: 1; or
an I at position D123 of SEQ ID NO: 1.

48. The KOD polymerase variant of claim 1, comprising two or more of the substitutions.

49. The KOD polymerase variant of claim 1, comprising three or more of the substitutions.

50.-53. (canceled)

54. The KOD polymerase variant of claim 42 comprising:
a W at position I16 of SEQ ID NO: 1; and
a P at position F116 of SEQ ID NO: 1.

55.-56. (canceled)

57. The KOD polymerase variant of claim 42 comprising:
a F at position I16 of SEQ ID NO: 1;
an I at position P90 of SEQ ID NO: 1; and
an I at position V93 of SEQ ID NO: 1.

58. (canceled)

59. The KOD polymerase variant of claim 42 comprising:
a F at position P90 of SEQ ID NO: 1;
a P at position F116 of SEQ ID NO:1; and
a M at position A117 of SEQ ID NO: 1.

60.-61. (canceled)

62. The KOD polymerase variant of claim 42 comprising:
a W and position I16 of SEQ ID NO: 1;
a H at position P36 of SEQ ID NO: 1; and
a P at position F116 of SEQ ID NO: 1.

63.-71. (canceled)

72. The KOD polymerase variant of claim 1 further comprising an Sso7d domain.

73. (canceled)

74. A method for amplifying a DNA template wherein the DNA template comprises one or more uracil and/or inosine nucleotides, the method comprising amplifying the DNA template using the KOD polymerase variant of claim 1.

75.-81. (canceled)
